(19) United States
(12) Patent Application Publication
(10) Pub. No.: US 2023/0103791 A1
(43) Pub. Date: Apr. 6, 2023

(54) 2,3-DIHYDROQUINAZOLIN COMPOUNDS AS NAV1.8 INHIBITORS

(71) Applicant: GLAXOSMITHKLINE INTELLECTUAL PROPERTY DEVELOPMENT LIMITED, Brentford, Middlesex (GB)

(72) Inventors: Roderick S. DAVIS, Collegeville, PA (US); Mark ELBAN, Collegeville, PA (US); Jie GUANG, Collegeville, PA (US); Ming-Hsun HO, Collegeville, PA (US); Tram H. HOANG, Collegeville, PA (US); William H. MILLER, Collegeville, PA (US); Joseph J. ROMANO, Collegeville, PA (US); Mythily VIMAL, Stevenage (GB); Maben YING, Collegeville, PA (US); David G. WASHBURN, Collegeville, PA (US)

(21) Appl. No.: 17/617,479

(22) PCT Filed: Jun. 23, 2020
(86) PCT No.: PCT/IB2020/055921

§ 371 Date: Dec. 8, 2021

Related U.S. Application Data
(60) Provisional application No. 62/867,714, filed on Jun. 27, 2019.
(60) Provisional application No. 62/896,698, filed on Sep. 6, 2019.

(51) Int. Cl. C07D 401/04; C07D 401/14; C07D 403/04; C07D 405/14; C07D 409/04; C07D 471/04; C07D 417/14; C07D 405/04; C07D 239/88; C07D 239/93; C07D 239/96

(52) U.S. Cl. CPC C07D 401/04; C07D 401/14; C07D 403/04; C07D 405/14; C07D 409/04; C07D 239/96; C07D 417/14; C07D 405/04; C07D 239/88; C07D 239/93; C07D 471/04

(57) ABSTRACT

The present invention relates to Naᵥ1.8 Inhibitor 2,3-dihydroquinazolin compounds of Formula (X):

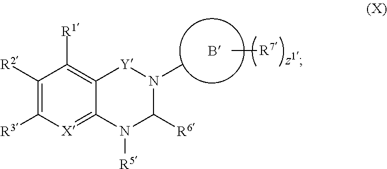

wherein Y′, X′, B′, R^{1′}, R^{2′}, R^{3′}, R^{5′}, R^{6′}, R^{7′}, and z¹ are as defined herein; or pharmaceutically acceptable salts or tautomer forms thereof, corresponding pharmaceutical compositions or formulations, methods or processes of compound preparation, methods, compounds for use in, uses for and/or combination therapies for treating pain and/or pain-related or associated disease(s), disorder(s) or condition(s), respectively.

### FIELD OF THE INVENTION

The present invention relates to Naᵥ1.8 Inhibitor 2,3-dihydroquinazolin compounds of Formula (X) or pharmaceutically acceptable salts or tautomer forms thereof, corresponding pharmaceutical compositions or formulations, methods or processes of compound preparation, methods, compounds for use in, uses for and/or combination therapies for treating pain-related or associated disease(s), disorder(s) or condition(s), respectively.

### BACKGROUND OF THE INVENTION

Pain is a protective mechanism by which animals avoid potential tissue damage, however there are numerous disease indications in which pain outlives its usefulness and becomes a disabling burden. Indications in which pain outlives its usefulness can be broadly categorized as those in which nerve damage or injury is the trigger (neuropathic pain), those in which an inflammatory response or metabolic dysregulation sensitizes the pain response (inflammatory pain) and those in which an injury or surgical procedure results in a short term elevation of pain response (post-operative/ambulatory pain).

Voltage-gated sodium channels underlie electrical signaling in all excitable tissues by setting the threshold and underlying the upstroke of action potentials. There are nine distinct isoforms of voltage-gated sodium channels. Those designated Naᵥ1.1, Naᵥ1.7, Naᵥ1.8 and Naᵥ1.9 are principally expressed on peripheral nerves where they control neuronal excitability. Naᵥ1.5 is the principle sodium channel isoform expressed in cardiac myocytes, Naᵥ1.4 is expressed and functions in skeletal muscle, whilst Naᵥ1.1, Naᵥ1.2, Naᵥ1.3 and Naᵥ1.6 are widely expressed in the central nervous system (CNS) and to an extent in the peripheral nervous system. The principal role of these nine voltage-gated sodium channels is comparable in that they control sodium influx into cells but their biophysical properties varies which greatly influences the physiological profile of their respective cell type (Catterall, 2012).

Currently, non-selective sodium channel inhibitors are utilized clinically as anti-arrhythmic and anti-seizure therapies, these include lidocaine, carbamazepine, amitriptyline and mexiletine. However, as these agents exhibit a lack of selectivity between the different sodium channel isoforms, their therapeutic utility is greatly reduced due to adverse side effects, largely mediated by activity in the CNS and heart. This has stimulated efforts to develop novel medicines which are selective for specific sodium channel isoforms in order to avoid side effects in the CNS and cardiovascular system.

The Naᵥ1.8 channel is expressed in neurons of the dorsal root ganglia (DRG) and highly expressed in the small diameter neurons of this tissue which form pain sensing C- and Aδ-nerve fibers (Abrahamsen, 2008; Amaya, 2000; Novakovic, 1998). The channel was proposed as a therapeutic target for analgesia as soon as it was originally cloned from rat DRG (Akopian, 1996) due to its prominent physiological role in this tissue type and restricted expression profile. Naᵥ1.8 was subsequently identified, cloned and characterized from human DRG tissue (Rabart 1998). The closest molecular relative of Naᵥ1.8 is Naᵥ1.5 which shares a sequence homology of ˜60%. Naᵥ1.8 was previously known as SNS (sensory neuron sodium channel), PN3 (peripheral nerve sodium channel type 3), and as it exhibits characteristic pharmacological properties in its resistant to block by tetrodotoxin, it is also described as a TTX-resistant sodium channel.

Support for Naᵥ1.8 as a therapeutic target for pain indications comes from several sources. Naᵥ1.8 has been shown to conduct the majority of current during upstroke of the action potential in DRG neurons (Blair & Bean, 2002) and due to its rate of re-priming is also critical for the ability of these neurons to fire repetitively (Blair and Bean, 2003). Increased expression and function of Naᵥ1.8 has been reported in response to painful stimuli such as inflammatory mediators (England 1996 & Gold 1996), nerve damage (Roza 2003 & Ruangsri 2011), and within painful neuromas (Black 2008 & Coward 2000). Knockout of the gene encoding Nav1.8 in mice resulted in a reduced pain phenotype in particular to inflammatory challenges (Akopian 1999). Knockdown of the mRNA encoding Naᵥ1.8 also resulted in reduced painful phenotypes in rodent models, particularly in neuropathic models (Lai 2002).

Pharmacological intervention via selective small molecule inhibitors has demonstrated efficacy in rodent models of inflammatory pain as well as neuropathic pain (Jarvis 2007 & Payne 2015). Supporting genetic evidence for Naᵥ1.8 is also present in patients with chronic neuropathic pain where multiple gain of function mutations has been reported to be causative in episodic painful neuropathies and small fiber neuropathies (Faber 2012, Han 2014 & Eijkenboom 2018).

Thus, there is a need for development of novel compounds of the present invention, particularly Naᵥ1.8 Inhibitor compounds or pharmaceutically acceptable salts thereof with novel mechanisms of action and pharmacological activity.

In light of the foregoing, a need exists to develop:
novel compounds or pharmaceutically acceptable salts and/or corresponding tautomeric forms thereof or corresponding pharmaceutical compositions thereof of the present invention, that exhibit pharmacological activities as Naᵥ1.8 Inhibitors;
methods for inhibiting a Naᵥ 1. 8 voltage-gated sodium channel in a subject;
methods for, compounds for use in and/or uses thereof, respectively, for treating:
   pain-associated disease(s), disorder(s) or condition(s), which include, but are not limited to: pain caused by a variety of diseases (as defined herein); pain caused by trauma; or pain caused by iatrogenic (i.e., such as medical or dental) procedures, or
   reducing or lessening severity of pain-associated disease(s), disorder(s) or condition(s) as herein; and/or
associated combination therapies for treating pain-associated disease(s), disorder(s) or condition(s), pain caused by trauma; or iatrogenic medical or dental procedure(s).

The present invention is directed to overcoming these and other problems encountered in the art.

### SUMMARY OF THE INVENTION

In general, the present invention relates to Naᵥ1.8 Inhibitor 2,3-dihydroquinazolin compounds of Formula (X) or pharmaceutically acceptable salts or tautomer forms thereof, corresponding pharmaceutical compositions or formulations, methods or processes of compound preparation, methods, compounds for use in, uses for and/or combination therapies for treating pain-related or associated disease(s), disorder(s) or condition(s), respectively.

In particular, the present invention relates to novel Naᵥ1.8 Inhibitor 2,3-dihydroquinazolin compounds of any of the Formulas disclosed herein, including Formula (X) and Formulas (I) to (III), respectively, or a pharmaceutically acceptable salt and/or a corresponding tautomer form thereof (i.e., including subgeneric formulas, as defined above) of the present invention and corresponding pharmaceutical compositions or formulations thereof.

The present invention also relates to processes for making compounds of any of the Formulas disclosed herein, including Formula (X) and Formulas (I) to (III) (i.e., including corresponding subgeneric formulas defined herein), respectively, or a pharmaceutically acceptable salt and/or a corresponding tautomer form thereof or corresponding pharmaceutical compositions thereof.

The present invention also relates to methods for treating, compounds for use in, uses for and/or combination therapies for pain-associated disease(s), disorder(s) or condition(s), such as pain caused by a variety of diseases, pain caused by trauma; or pain caused by iatrogenic (i.e., such as medical or dental) procedures.

### DETAILED DESCRIPTION OF THE INVENTION

The present invention relates to Naᵥ1.8 Inhibitor 2,3-dihydroquinazolin compounds of Formula (X) or pharmaceutically acceptable salts or tautomer forms thereof, corresponding pharmaceutical compositions or formulations, methods or processes of compound preparation, methods, compounds for use in, uses for and/or combination therapies for treating pain and/or pain-related or associated disease(s), disorder(s) or condition(s), respectively.

### Compounds

In particular, the present invention relates to novel Naᵥ1.8 Inhibitor 2,3-dihydroquinazolin compounds of any of the Formulas disclosed herein, including Formula (X) and Formulas (I) to (III) (i.e., including subgeneric formulas, as defined herein), respectively, or a pharmaceutically acceptable salt and/or a corresponding tautomer form thereof.

In another aspect, it is understood that different aspects of the present invention as defined throughout the present application may be adapted to use or encompass compounds of the Formulas as defined above throughout the present application.

In one aspect, the present invention relates to a compound of Formula (X):

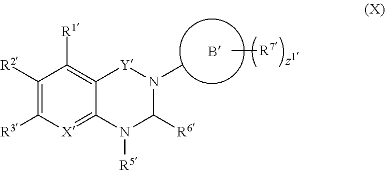

where:

Y′ is selected from: CH₂, C═O and C═S;

X′ is N or C—R⁴;

wherein:

R^{4′} is selected from: hydrogen, halogen, —C≡N, —NR^{a}R^{b}, straight or branched-(C₁₋₆)-alkyl, —OR^{c} and —S(O)ₚR^{d},
wherein, when R^{4′} is straight or branched —(C₁₋₆)-alkyl or —OR^{c}, the alkyl chain, when present, is optionally substituted with one to six substituents independently selected from: halogen, —C≡N, oxo, —NR^{a}R^{b}, straight or branched-(C₁₋₆)-alkyl, straight or branched-(C₁₋₆)-haloalkyl and —OR^{c};

R^{1′}, R^{2′} and R^{3′} are independently selected from: hydrogen, halogen, —C≡N, —NR^{a}R^{b}, straight or branched-(C₁₋₆)-alkyl, carbocyclic, heterocyclic, bicycloalkyl, —OR^{c} and —S(O)ₚR^{d},
wherein, when any of R^{1′}, R^{2′} and R^{3′} is a straight or branched-(C₁₋₆)-alkyl, or —OR^{c}, the alkyl chain, when present, is optionally substituted with one to six substituents independently selected from: halogen, —C≡N, —NR^{a}R^{b}, straight or branched-(C₁₋₆)-alkyl, straight or branched-(C₁₋₆)-haloalkyl, oxo and —OR^{c};

R^{5′} is selected from: carbocyclic, —CH₂-unsaturated carbocyclic, heterocyclic, or bicycloalkyl,
wherein, R^{5′} is optionally substituted with from one to four substituents independently selected from: —C≡N, —NR^{a}R^{b}, halogen, oxo, —C(O)NHR^{a}, —C(O)NR^{a}R^{b}, straight or branched-(C₁₋₆)-alkyl, —OR^{c}, and (C₃₋₆)cycloalkyl,
   wherein each of: straight or branched-(C₁₋₆)-alkyl, the alkyl chain of —OR^{c}, when present, and (C₃₋₆)-cycloalkyl is optionally substituted with one to six substituents independently selected from: halogen, oxo, —OH, —NH₂, —NHC₁₋₄alkyl, —N(C₁₋₄alkyl)₂, —OC₁₋₄alkyl and —OC₁₋₄alkyl substituted with from 1 to 6 substituents independently selected from: fluoro, oxo, —OH, —NH₂, —NHC₁₋₄alkyl, —N(C₁₋₄alkyl)₂, —OC₁₋₃alkyl, and —OC₁₋₃alkyl substituted 1 to 6 times by fluoro;

R^{6′} is hydrogen, oxo, straight or branched-(C₁₋₆)-alkyl or straight or branched-(C₁₋₆)-haloalkyl;

B′ is selected from: aryl, heterocycloalkyl, and heteroaryl;

each R^{7′} is independently selected from: halogen, oxo, —C≡N, —NR^{a}R^{b}, —OR^{c}, —S(O)ₚR^{d}, straight or branched (C₁₋₆) alkyl, bicycloalkyl and (C₃₋₆)-cycloalkyl,
wherein, when R^{7′} is a straight or branched —(C₁₋₆)-alkyl, or —OR^{c}, the alkyl chain, when present, is optionally substituted with one to three substituents independently selected from: halogen, —C≡N, —NR^{a}R^{b}, straight or branched-(C₁₋₆)-alkyl, straight or branched-(C₁₋₆)-haloalkyl, oxo and —OR^{c};

R^{d} is hydrogen, —OH, —NR^{a}R^{b}, straight or branched-(C₁₋₆)-alkyl, straight or branched-(C₁₋₆)-haloalkyl or (C₃₋₆)-cycloalkyl;

in each occurrence, R^{a}, R^{b} and R^{c} are independently selected from: hydrogen, straight or branched-(C₁₋₆)-alkyl, and (C₃₋₆)-cycloalkyl,
wherein each of: straight or branched-(C₁₋₆)-alkyl, and (C₃₋₆)-cycloalkyl is optionally substituted with one to six substituents independently selected from: halogen, oxo, —OH, —NH₂, —NHC₁₋₄alkyl, —N(C₁₋₄alkyl)₂, —OC₁₋₄alkyl and —OC₁₋₄alkyl substituted with from 1 to 6 substituents independently selected from: fluoro, oxo, —OH, —NH₂, —NHC₁₋₄alkyl, —N(C₁₋₄alkyl)₂, —OC₁₋₃alkyl, and —OC₁₋₃alkyl substituted 1 to 6 times by fluoro;

z^{1′} is an integer from 0 to 5; and

p is 0, 1 or 2;

or a pharmaceutically acceptable salt and/or a corresponding tautomer form thereof.

For compounds of Formula (X), suitably Y′ is CH₂.

For compounds of Formula (X), suitably Y′ is C═O.

For compounds of Formula (X), suitably Y′ is C═S.

For compounds of Formula (X), suitably when Y′ is C═O, R^{6′} is hydrogen.

For compounds of Formula (X), suitably X′ is N.

For compounds of Formula (X), suitably X′ is C—H.

For compounds of Formula (X), suitably X′ is C—CH₃.

For compounds of Formula (X), suitably X′ is C—F.

For compounds of Formula (X), suitably X′ is C—Cl.

For compounds of Formula (X), suitably X′ is C—Br.

For compounds of Formula (X), suitably R^{1′}, R^{2′} and R^{3′} are independently selected from: hydrogen, fluoro, chloro, bromo, —CH₃, —CF₃, —CHF₂, —OCH₃, —OCH₂CF₃, —OCHF₂, —OCF₃, —CF₂CH₂OH, —N(CH₃)₂, —NHCH₃, —C≡N, —OH, —S(O)₂CH₃, cyclopropyl, and oxetanyl.

For compounds of Formula (X), suitably R^{1′}, R^{2′} and R^{3′} are independently selected from: hydrogen, fluoro, chloro, bromo, —C≡N, —CH₃, —CF₃, —CHF₂, —OCH₃, —OCH₂CF₃, —OCHF₂, —OCF₃, and —CF₂CH₂OH.

For compounds of Formula (X), suitably R^{5′} is selected from: phenyl, cyclopentyl, cyclohexyl,

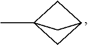

thiophenyl, thiazolyl, pyridyl, tetrahydropyranyl, and —CH₂-phenyl,
wherein, R^{5′} is optionally substituted with from one to four substituents independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(CH₃)₂, —NHCH₃, —C≡N, —OH, and cyclopropyl.

For compounds of Formula (X), suitably R^{5′} is phenyl, where phenyl is
optionally substituted with from one to four substituents independently selected from: —C≡N, —NR^{a}R^{b}, halogen, oxo, —C(O)NHR^{a}, —C(O)NR^{a}R^{b}, straight or branched-(C₁₋₆)-alkyl, —OR^{c}, and (C₃₋₆)cycloalkyl,
   wherein each of: straight or branched-(C₁₋₆)-alkyl, the alkyl chain of —OR^{c}, when present, and (C₃₋₆)-cycloalkyl is optionally substituted with one to six substituents independently selected from: halogen, oxo, —OH, —NH₂, —NHC₁₋₄alkyl, —N(C₁₋₄alkyl)₂, —OC₁₋₄alkyl and —OC₁₋₄alkyl substituted with from 1 to 6 substituents independently selected from: fluoro, oxo, —OH, —NH₂, —NHC₁₋₄alkyl, —N(C₁₋₄alkyl)₂, —OC₁₋₃alkyl, and —OC₁₋₃alkyl substituted 1 to 6 times by fluoro.

For compounds of Formula (X), suitably R^{5′} is phenyl,
wherein, R^{5′} is substituted with one or two substituents independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —OCH₂CH₃, —OCHF₂, —OCF₃, and —OCH₂CH₂OH.

For compounds of Formula (X), suitably R^{6′} is hydrogen, oxo or —CH₃.

For compounds of Formula (X), suitably R^{6′} is hydrogen.

For compounds of Formula (X), suitably B′ is selected from: pyridinyl, pyrimidinyl, phenyl, pyridazinyl, tetrahydrothiophenyl, pyrazolyl, piperidinyl, tetrahydrothiopyranyl, dihydropyrimidinyl, tetrahydropyridinyl, pyrazinyl, furanyl and hexahydropyrimidinyl.

For compounds of Formula (X), suitably B′ is pyridinyl.

For compounds of Formula (X), suitably B′ is selected from:

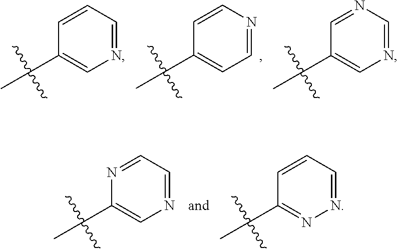 [SMILES: *c1cccnc1 ; *c1ccncc1 ; *c1cncnc1 ; *c1cnccn1 ; *c1cccnn1]

For compounds of Formula (X), suitably each R^{7′} is independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(C₁₋₄alkyl)₂, —NH(C₁₋₄)alkyl, —C≡N, —OH, oxo, —C(O)OH, —C(O)CH₃, —OCH₂C(O)OH, —NC(O)CH₃, —NHCH₂CH₂OH, —S(O)₂CH₃, —S(O)₂NH₂, and cyclopropyl.

For compounds of Formula (X), suitably each R^{7′} is independently selected from: fluoro, chloro, —CH₃, —CH₂CH₃, —CH₂CF₃, —CH(CH₃)₂, —C(CH₃)₃, —CF₃, —CF₂CH₂OH, —C(O)NH₂, —OCH₃, —OCH₂CH₃, —OCHF₂, —OCF₃, oxo, —C(O)OH, —C(O)CH₃, and —OCH₂C(O)OH.

For compounds of Formula (X), suitably each R^{7′} is independently selected from: —CH₃ and oxo.

For compounds of Formula (X), suitably Z^{1′} is an integer from 1 to 4, suitably an integer from 1 to 3, suitably an integer selected from 1 and 2.

In one aspect, the present invention relates to a compound of Formula (XI):

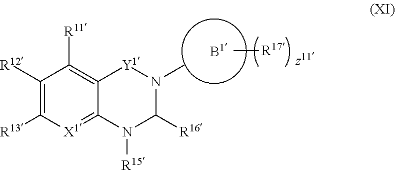

where:

Y^{1′} is selected from: CH₂, C═O and C═S;

X^{1′} is N or C—R^{14′};

wherein:

R^{14′} is selected from: hydrogen, halogen, —C≡N, —NR^{a}R^{b}, straight or branched-(C₁₋₆)-alkyl, —OR^{c} and —S(O)ₚR^{d},
wherein, when R^{4′} is straight or branched —(C₁₋₆)-alkyl or —OR^{c}, the alkyl chain, when present, is optionally substituted with one to six substituents independently selected from: fluoro, chloro, and bromo;
R^{11′}, R^{12′} and R^{13′} are independently selected from: hydrogen, halogen, —C≡N, —NR^{a}R^{b}, straight or branched-(C₁₋₆)-alkyl, cycloakyl, heterocycloalkyl, heteroaryl, —OR^{c} and —S(O)ₚR^{d},
   wherein, when any of R^{11′}, R^{12′} and R^{13′} is a straight or branched-(C₁₋₆)-alkyl, or —OR^{c}, the alkyl chain, when present, is optionally substituted with one to six substituents independently selected from: halogen, —NR^{a}R^{b}, straight or branched-(C₁₋₆)-alkyl, straight or branched-(C₁₋₆)-haloalkyl and —OR^{c};

R^{15′} is selected from: phenyl, cycloalkyl, —CH₂-phenyl, heterocycloalkyl, heteroaryl, and bicycloalkyl,
wherein, R^{15′} is optionally substituted with from one to four substituents independently selected from: —C≡N, —NR^{a}R^{b}, halogen, oxo, —C(O)NHR^{a}, —C(O)NR^{a}R^{b}, straight or branched-(C₁₋₆)-alkyl, —OR^{c}, and (C₃₋₆)cycloalkyl,
   wherein each of: straight or branched-(C₁₋₆)-alkyl, the alkyl chain of —OR^{c}, when present, and (C₃₋₆)-cycloalkyl is optionally substituted with one to six substituents independently selected from: halogen, oxo, —OH, —NH₂, —NHC₁₋₂alkyl, and —N(C₁₋₂alkyl)₂;

R^{16′} is hydrogen, oxo, straight or branched-(C₁₋₄)-alkyl or straight or branched-(C₁₋₄)-haloalkyl;

B^{1′} is selected from: phenyl, heterocycloalkyl, and heteroaryl;

each R^{17′} is independently selected from: halogen, oxo, —C≡N, —NR^{a}R^{b}, —OR^{c}, —S(O)ₚR^{d}, straight or branched (C₁₋₆) alkyl, and (C₃₋₆)-cycloalkyl,
wherein, when R is a straight or branched —(C₁₋₆)-alkyl, or —OR^{c}, the alkyl chain, when present, is optionally substituted with one to three substituents independently selected from: halogen, —NR^{a}R^{b}, oxo and —OR^{c};

R^{d} is hydrogen, —OH, —NR^{a}R^{b}, straight or branched-(C₁₋₆)-alkyl, straight or branched-(C₁₋₆)-haloalkyl or (C₃₋₆)-cycloalkyl;

in each occurrence, R^{a}, R^{b} and R^{c} are independently selected from: hydrogen, straight or branched-(C₁₋₆)-alkyl, and (C₃₋₆)-cycloalkyl,
wherein each of: straight or branched-(C₁₋₆)-alkyl, and (C₃₋₆)-cycloalkyl is optionally substituted with one to six substituents independently selected from: halogen, oxo, —OH, —NH₂, —NHC₁₋₄alkyl, and —N(C₁₋₄alkyl)₂;
z^{11′} is an integer from 0 to 5; and
p is 0, 1 or 2;
or a pharmaceutically acceptable salt and/or a corresponding tautomer form thereof.

For compounds of Formula (XI), suitably Y^{1′} is CH₂.

For compounds of Formula (XI), suitably Y^{1′} is C═O.

For compounds of Formula (XI), suitably Y^{1′} is C═S.

For compounds of Formula (XI), suitably when Y^{1′} is C═O, R^{16′} is hydrogen.

For compounds of Formula (XI), suitably X^{1′} is N.

For compounds of Formula (XI), suitably X^{1′} is C—H.

For compounds of Formula (XI), suitably X^{1′} is C—CH₃.

For compounds of Formula (XI), suitably X^{1′} is C—F.

For compounds of Formula (XI), suitably X^{1′} is C—Cl.

For compounds of Formula (XI), suitably X^{1′} is C—Br.

For compounds of Formula (XI), suitably R^{11′}, R^{12′} and R^{13′} are independently selected from: hydrogen, fluoro, chloro, bromo, —CH₃, —CF₃, —CHF₂, —OCH₃, —OCH₂CF₃, —OCHF₂, —OCF₃, —CF₂CH₂OH, —N(CH₃)₂, —NHCH₃, —C≡N, —OH, —S(O)₂CH₃, cyclopropyl, and oxetanyl.

For compounds of Formula (XI), suitably R^{11′}, R^{12′} and R^{13′} are independently selected from: hydrogen, fluoro, chloro, bromo, —C≡N, —CH₃, —CF₃, —CHF₂, —OCH₃, —OCH₂CF₃, —OCHF₂, —OCF₃, and —CF₂CH₂OH.

For compounds of Formula (XI), suitably R^{15′} is selected from: phenyl, cyclopentyl, cyclohexyl,

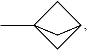

thiophenyl, thiazolyl, pyridyl, tetrahydropyranyl, and —CH₂-phenyl,
wherein, R^{15′} is optionally substituted with from one to four substituents independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(CH₃)₂, —NHCH₃, —C≡N, —OH, and cyclopropyl.

For compounds of Formula (XI), suitably R^{15′} is phenyl,
wherein, R^{15′} is substituted with one or two substituents independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —OCH₂CH₃, —OCHF₂, —OCF₃, and —OCH₂CH₂OH.

For compounds of Formula (XI), suitably R^{16′} is hydrogen, oxo or —CH₃.

For compounds of Formula (XI), suitably R^{16′} is hydrogen.

For compounds of Formula (XI), suitably B^{1′} is selected from: pyridinyl, pyrimidinyl, phenyl, pyridazinyl, tetrahydrothiophenyl, pyrazolyl, piperidinyl, tetrahydrothiopyranyl, dihydropyrimidinyl, tetrahydropyridinyl, pyrazinyl, furanyl and hexahydropyrimidinyl.

For compounds of Formula (XI), suitably B^{1′} is pyridinyl.

For compounds of Formula (XI), suitably B^{1′} is selected from:

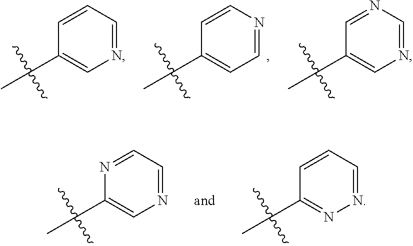 [SMILES: *c1cccnc1 ; *c1ccncc1 ; *c1cncnc1 ; *c1cnccn1 ; *c1cccnn1]

For compounds of Formula (XI), suitably each R^{17′} is independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(C₁₋₄alkyl)₂, —NH(C₁₋₄)alkyl, —C≡N, —OH, oxo, —C(O)OH, —C(O)CH₃, —OCH₂C(O)OH, —NC(O)CH₃, —NHCH₂CH₂OH, —S(O)₂CH₃, —S(O)₂NH₂, and cyclopropyl.

For compounds of Formula (XI), suitably each R^{17′} is independently selected from: fluoro, chloro, —CH₃, —CH₂CH₃, —CH₂CF₃, —CH(CH₃)₂, —C(CH₃)₃, —CF₃, —CF₂CH₂OH, —C(O)NH₂, —OCH₃, —OCH₂CH₃, —OCHF₂, —OCF₃, oxo, —C(O)OH, —C(O)CH₃, and —OCH₂C(O)OH.

For compounds of Formula (XI), suitably each R^{17′} is independently selected from: —CH₃ and oxo.

For compounds of Formula (XI), suitably Z^{11′} is an integer from 1 to 4, suitably an integer from 1 to 3, suitably an integer selected from 1 and 2.

In one aspect, the present invention relates to a compound of Formula (XII):

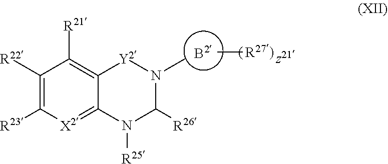

where:

Y^{2′} is selected from: CH₂, C═O and C═S;

X^{2′} is N or C—R^{24′};

wherein:

R^{24′} is selected from: hydrogen, fluoro, chloro, bromo, and —CH₃;

R^{21′}, R^{22′} and R^{22′} are independently selected from: hydrogen, fluoro, chloro, bromo, —CH₃, —CF₃, —CHF₂, —OCH₃, —OCH₂CF₃, —OCHF₂, —OCF₃, —CF₂CH₂OH, —N(CH₃)₂, —NHCH₃, —C≡N, —OH, —S(O)₂CH₃, cyclopropyl, and oxetanyl;

R^{25′} is selected from: phenyl, cyclopentyl, cyclohexyl,

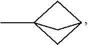

thiophenyl, thiazolyl, pyridyl, tetrahydropyranyl, and —CH₂-phenyl,
wherein, R^{25′} is optionally substituted with from one to four substituents independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(CH₃)₂, —NHCH₃, —C≡N, —OH, and cyclopropyl;

R^{26′} is hydrogen, oxo, or —CH₃;

B^{2′} is selected from: pyridinyl, pyrimidinyl, phenyl, pyridazinyl, tetrahydrothiophenyl, pyrazolyl, piperidinyl, tetrahydrothiopyranyl, dihydropyrimidinyl, tetrahydropyridinyl, pyrazinyl, furanyl and hexahydropyrimidinyl;
each R^{27′} is independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(C₁₋₄alkyl)₂, —NH(C₁₋₄)alkyl, —C≡N, —OH, —C(O)OH, —C(O)CH₃, oxo, —OCH₂C(O)OH, —NC(O)CH₃, —NHCH₂CH₂OH, —S(O)₂CH₃, —S(O)₂NH₂, and cyclopropyl; and

z^{21′} is an integer from 0 to 4;
or a pharmaceutically acceptable salt and/or a corresponding tautomer form thereof.

For compounds of Formula (XII), suitably Y^{2′} is CH₂.

For compounds of Formula (XII), suitably Y^{2′} is C═O.

For compounds of Formula (XII), suitably Y^{2′} is C═S.

For compounds of Formula (XII), suitably when Y^{2′} is C═O, R^{26′} is hydrogen.

For compounds of Formula (XII), suitably X^{2′} is N.

For compounds of Formula (XII), suitably X^{2′} is C—H.

For compounds of Formula (XII), suitably X^{2′} is C—CH₃.

For compounds of Formula (XII), suitably X^{2′} is C—F.

For compounds of Formula (XII), suitably X^{2′} is C—Cl.

For compounds of Formula (XII), suitably X^{2′} is C—Br.

For compounds of Formula (XII), suitably R^{21′}, R^{22′} and R^{23′} are independently selected from: hydrogen, fluoro, chloro, bromo, —CH₃, —CF₃, —CHF₂, —OCH₃, —OCH₂CF₃, —OCHF₂, —OCF₃, —CF₂CH₂OH, —N(CH₃)₂, —NHCH₃, —C≡N, —OH, —S(O)₂CH₃, cyclopropyl, and oxetanyl.

For compounds of Formula (XII), suitably R^{21′}, R^{22′} and R^{23′} are independently selected from: hydrogen, fluoro, chloro, bromo, —C≡N, —CH₃, —CF₃, —CHF₂, —OCH₃, —OCH₂CF₃, —OCHF₂, —OCF₃, and —CF₂CH₂OH.

For compounds of Formula (XII), suitably R^{25′} is selected from: phenyl, cyclopentyl, cyclohexyl,

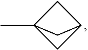

thiophenyl, thiazolyl, pyridyl, tetrahydropyranyl, and —CH₂-phenyl,
wherein, R^{25′} is optionally substituted with from one to four substituents independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(CH₃)₂, —NHCH₃, —C≡N, —OH, and cyclopropyl.

For compounds of Formula (XII), suitably R^{25′} is phenyl,
wherein, R^{25′} is substituted with one or two substituents independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —OCH₂CH₃, —OCHF₂, —OCF₃, and —OCH₂CH₂OH.

For compounds of Formula (XII), suitably R^{26′} is hydrogen, oxo or —CH₃.

For compounds of Formula (XII), suitably R^{26′} is hydrogen.

For compounds of Formula (XII), suitably B^{2′} is selected from: pyridinyl, pyrimidinyl, phenyl, pyridazinyl, tetrahydrothiophenyl, pyrazolyl, piperidinyl, tetrahydrothiopyranyl, dihydropyrimidinyl, tetrahydropyridinyl, pyrazinyl, furanyl and hexahydropyrimidinyl.

For compounds of Formula (XII), suitably B^{2′} is pyridinyl.

For compounds of Formula (XII), suitably B^{2′} is selected from:

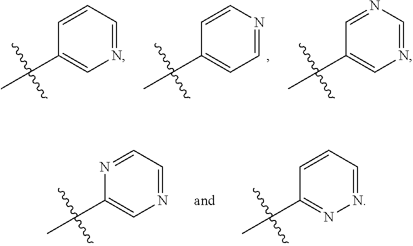 [SMILES: *c1cccnc1 ; *c1ccncc1 ; *c1cncnc1 ; *c1cnccn1 ; *c1cccnn1]

For compounds of Formula (XII), suitably each R^{27′} is independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(C₁₋₄alkyl)₂, —NH(C₁₋₄)alkyl, —C≡N, —OH, oxo, —C(O)OH, —C(O)CH₃, —OCH₂C(O)OH, —NC(O)CH₃, —NHCH₂CH₂OH, —S(O)₂CH₃, —S(O)₂NH₂, and cyclopropyl.

For compounds of Formula (XII), suitably each R^{27′} is independently selected from: fluoro, chloro, —CH₃, —CH₂CH₃, —CH₂CF₃, —CH(CH₃)₂, —C(CH₃)₃, —CF₃, —CF₂CH₂OH, —C(O)NH₂, —OCH₃, —OCH₂CH₃, —OCHF₂, —OCF₃, oxo, —C(O)OH, —C(O)CH₃, and —OCH₂C(O)OH.

For compounds of Formula (XII), suitably each R^{27′} is independently selected from: —CH₃ and oxo.

For compounds of Formula (XII), suitably Z^{21′} is an integer from 1 to 4, suitably an integer from 1 to 3, suitably an integer selected from 1 and 2.

In one aspect, the present invention relates to a compound of Formula (XIII):

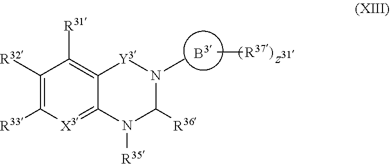

where:

Y^{3′} is selected from: CH₂, C═O and C═S;

X^{3′} is N or C—R^{34′};

wherein:

R^{34′} is selected from: hydrogen, fluoro, chloro, bromo, and —CH₃;

R^{31′}, R^{32′} and R^{33′} are independently selected from: hydrogen, fluoro, chloro, bromo, —CH₃, —CF₃, —CHF₂, —OCH₃, —OCH₂CF₃, —OCHF₂, —OCF₃, —CF₂CH₂OH, —N(CH₃)₂, —NHCH₃, —C≡N, —OH, —S(O)₂CH₃, cyclopropyl, and oxetanyl;

R^{35′} is selected from: phenyl, cyclopentyl, cyclohexyl,

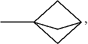

thiophenyl, thiazolyl, pyridyl, tetrahydropyranyl, and —CH₂-phenyl,
wherein, R^{35′} is optionally substituted with from one to four substituents independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(CH₃)₂, —NHCH₃, —C≡N, —OH, and cyclopropyl;

R^{36′} is hydrogen, oxo, or —CH₃;

B^{3′} is selected from: pyridinyl, pyrimidinyl, phenyl, pyridazinyl, tetrahydrothiophenyl, pyrazolyl, piperidinyl, tetrahydrothiopyranyl, dihydropyrimidinyl, tetrahydropyridinyl, pyrazinyl, furanyl and hexahydropyrimidinyl;
each R^{37′} is independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(C₁₋₄alkyl)₂, —NH(C₁₋₄)alkyl, —C≡N, —OH, —C(O)OH, —C(O)CH₃, oxo, —OCH₂C(O)OH, —NC(O)CH₃, —NHCH₂CH₂OH, —S(O)₂CH₃, —S(O)₂NH₂, and cyclopropyl; and

Z^{31′} is an integer from 0 to 4;
provided that when Y^{3′} is O, R^{36′} is hydrogen;
or a pharmaceutically acceptable salt and/or a corresponding tautomer form thereof.

For compounds of Formula (XIII), suitably Y^{3′} is CH₂.

For compounds of Formula (XIII), suitably Y^{3′} is C═O.

For compounds of Formula (XIII), suitably Y^{3′} is C═S.

For compounds of Formula (XIII), suitably X^{3′} is N.

For compounds of Formula (XIII), suitably X^{3′} is C—H.

For compounds of Formula (XIII), suitably X^{3′} is C—CH₃.

For compounds of Formula (XIII), suitably X^{3′} is C—F.

For compounds of Formula (XIII), suitably X^{3′} is C—Cl.

For compounds of Formula (XIII), suitably X^{3′} is C—Br.

For compounds of Formula (XIII), suitably R^{31′}, R^{32′} and R^{33′} are independently selected from: hydrogen, fluoro, chloro, bromo, —CH₃, —CF₃, —CHF₂, —OCH₃, —OCH₂CF₃, —OCHF₂, —OCF₃, —CF₂CH₂OH, —N(CH₃)₂, —NHCH₃, —C≡N, —OH, —S(O)₂CH₃, cyclopropyl, and oxetanyl.

For compounds of Formula (XIII), suitably R^{31′}, R^{32′} and R^{33′} are independently selected from: hydrogen, fluoro, chloro, bromo, —C≡N, —CH₃, —CF₃, —CHF₂, —OCH₃, —OCH₂CF₃, —OCHF₂, —OCF₃, and —CF₂CH₂OH.

For compounds of Formula (XIII), suitably R³¹ and R³³ are hydrogen, and R³² is selected from: hydrogen, fluoro, chloro, bromo, —C≡N, —CH₃, —CF₃, —CHF₂, —OCH₃, —OCH₂CF₃, —OCHF₂, —OCF₃, and —CF₂CH₂OH.

For compounds of Formula (XIII), suitably R^{35′} is selected from: phenyl, cyclopentyl, cyclohexyl,

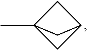

thiophenyl, thiazolyl, pyridyl, tetrahydropyranyl, and —CH₂-phenyl,
wherein, R^{35′} is optionally substituted with from one to four substituents independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(CH₃)₂, —NHCH₃, —C≡N, —OH, and cyclopropyl.

For compounds of Formula (XIII), suitably R^{35′} is phenyl,
wherein, R^{35′} is substituted with one or two substituents independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —OCH₂CH₃, —OCHF₂, —OCF₃, and —OCH₂CH₂OH.

For compounds of Formula (XIII), suitably R^{35′} is phenyl,
wherein, R^{35′} is substituted with one or two substituents independently selected from: fluoro, —CH₃, and —OCH₃.

For compounds of Formula (XIII), suitably R^{36′} is hydrogen, oxo or —CH₃.

For compounds of Formula (XIII), suitably R^{36′} is hydrogen.

For compounds of Formula (XIII), suitably B^{3′} is selected from: pyridinyl, pyrimidinyl, phenyl, pyridazinyl, tetrahydrothiophenyl, pyrazolyl, piperidinyl, tetrahydrothiopyranyl, dihydropyrimidinyl, tetrahydropyridinyl, pyrazinyl, furanyl and hexahydropyrimidinyl.

For compounds of Formula (XIII), suitably B^{3′} is pyridinyl.

For compounds of Formula (XIII), suitably B^{3′} is selected from:

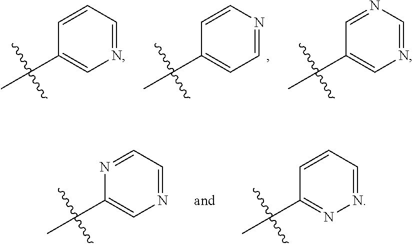 [SMILES: *c1cccnc1 ; *c1ccncc1 ; *c1cncnc1 ; *c1cnccn1 ; *c1cccnn1]

For compounds of Formula (XIII), suitably each R^{37′} is independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(C₁₋₄alkyl)₂, —NH(C₁₋₄)alkyl, —C≡N, —OH, oxo, —C(O)OH, —C(O)CH₃, —OCH₂C(O)OH, —NC(O)CH₃, —NHCH₂CH₂OH, —S(O)₂CH₃, —S(O)₂NH₂, and cyclopropyl.

For compounds of Formula (XIII), suitably each R^{37′} is independently selected from: fluoro, chloro, —CH₃, —CH₂CH₃, —CH₂CF₃, —CH(CH₃)₂, —C(CH₃)₃, —CF₃, —CF₂CH₂OH, —C(O)NH₂, —OCH₃, —OCH₂CH₃, —OCHF₂, —OCF₃, oxo, —C(O)OH, —C(O)CH₃, and —OCH₂C(O)OH.

For compounds of Formula (XIII), suitably each R^{37′} is independently selected from: —CH₃ and oxo.

For compounds of Formula (XIII), suitably Z^{31′} is an integer from 1 to 4, suitably an integer from 1 to 3, suitably an integer selected from 1 and 2.

In one aspect, the present invention relates to a compound of Formula (XIV):

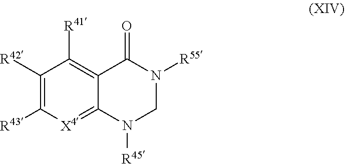

where:

X^{4′} is N or C—R^{44′};

wherein:

R^{44′} is selected from: hydrogen, fluoro, chloro, bromo, and —CH₃;

R^{41′}, R^{42′} and R^{43′} are independently selected from: hydrogen, fluoro, chloro, bromo, —CH₃, —CF₃, —CHF₂, —OCH₃, —OCH₂CF₃, —OCHF₂, —OCF₃, —CF₂CH₂OH, —N(CH₃)₂, —NHCH₃, —C≡N, —OH, —S(O)₂CH₃, cyclopropyl, and oxetanyl;

R^{45′} is selected from: phenyl, cyclopentyl, cyclohexyl,

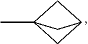

thiophenyl, thiazolyl, pyridyl, tetrahydropyranyl, and —CH₂-phenyl,
wherein, R^{45′} is optionally substituted with from one to four substituents independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(CH₃)₂, —NHCH₃, —C≡N, —OH, and cyclopropyl; and

R^{55′} is selected from:

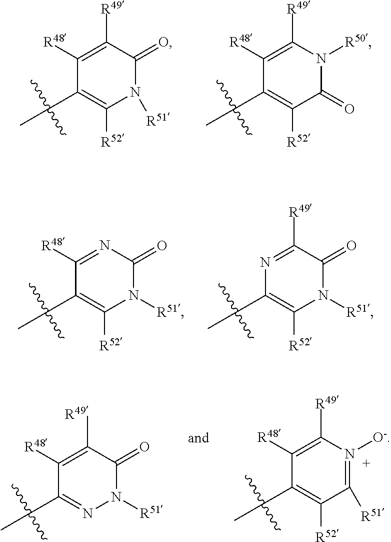

or a corresponding tautomer form thereof,
wherein, R^{48′}, R^{49′}, R^{50′}, R^{51′} and R^{52′} are independently selected from: hydrogen, fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(C₁₋₄alkyl)₂, —NH(C₁₋₄)alkyl, —C≡N, —OH, —C(O)OH, —C(O)CH₃, —OCH₂C(O)OH, —NC(O)CH₃, —NHCH₂CH₂OH, —S(O)₂CH₃, —S(O)₂NH₂, and cyclopropyl;

or a pharmaceutically acceptable salt and/or a corresponding tautomer form thereof.

For compounds of Formula (XIV), suitably X^{4′} is N.

For compounds of Formula (XIV), suitably X^{4′} is C—H.

For compounds of Formula (XIV), suitably X^{4′} is C—CH₃.

For compounds of Formula (XIV), suitably X^{4′} is C—F.

For compounds of Formula (XIV), suitably X^{4′} is C—Cl.

For compounds of Formula (XIV), suitably X^{4′} is C—Br.

For compounds of Formula (XIV), suitably R^{41′}, R^{42′} and R^{43′} are independently selected from: hydrogen, fluoro, chloro, bromo, —CH₃, —CF₃, —CHF₂, —OCH₃, —OCH₂CF₃, —OCHF₂, —OCF₃, —CF₂CH₂OH, —N(CH₃)₂, —NHCH₃, —C≡N, —OH, —S(O)₂CH₃, cyclopropyl, and oxetanyl.

For compounds of Formula (XIV), suitably R^{41′}, R^{42′} and R^{43′} are independently selected from: hydrogen, fluoro, chloro, bromo, —C≡N, —CH₃, —CF₃, —CHF₂, —OCH₃, —OCH₂CF₃, —OCHF₂, —OCF₃, and —CF₂CH₂OH.

For compounds of Formula (XIV), suitably R⁴¹ and R⁴³ are hydrogen and R⁴² selected from: hydrogen, —CH₃, fluoro, chloro, bromo, —C≡N, —CF₃, —CHF₂, —OCH₃, —OCH₂CF₃, —OCHF₂, —OCF₃, and —CF₂CH₂OH.

For compounds of Formula (XIV), suitably R^{45′} is selected from: phenyl, cyclopentyl, cyclohexyl,

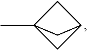

thiophenyl, thiazolyl, pyridyl, tetrahydropyranyl, and —CH₂-phenyl,
wherein, R^{45′} is optionally substituted with from one to four substituents independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(CH₃)₂, —NHCH₃, —C≡N, —OH, and cyclopropyl.

For compounds of Formula (XIV), suitably R^{45′} is phenyl,
wherein, R^{45′} is substituted with one or two substituents independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —OCH₂CH₃, —OCHF₂, —OCF₃, and —OCH₂CH₂OH.

For compounds of Formula (XIV), suitably R^{55′} is selected from:

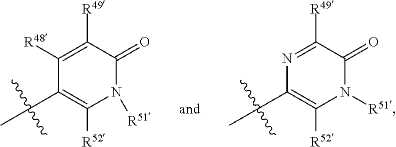

or a corresponding tautomer form thereof,
wherein, R^{48′}, R^{49′}, R^{51′} and R^{52′} are independently selected from: hydrogen, fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(C₁₋₄alkyl)₂, —NH(C₁₋₄)alkyl, —C≡N, —OH, —C(O)OH, —C(O)CH₃, —OCH₂C(O)OH, —NC(O)CH₃, —NHCH₂CH₂OH, —S(O)₂CH₃, —S(O)₂NH₂, and cyclopropyl;

For compounds of Formula (XIV), suitably R^{55′} is selected from:

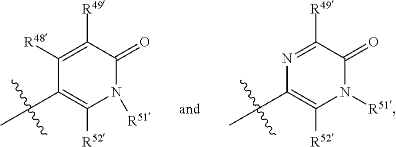

or a corresponding tautomer form thereof,
wherein, R^{48′}, R^{49′}, R^{51′} and R^{52′} are independently selected from: hydrogen, fluoro, chloro, —CH₃, —CH₂CH₃, —CH₂CF₃, —CH(CH₃)₂, —C(CH₃)₃, —CF₃, —CF₂CH₂OH, —C(O)NH₂, —OCH₃, —OCH₂CH₃, —OCHF₂, —OCF₃, —C(O)OH, —C(O)CH₃, and —OCH₂C(O)OH.

In one aspect, the present invention relates to a compound of Formula (I):

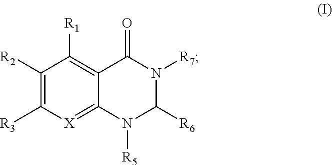

where:

X is N or C—R⁴;

R¹, R² or R³ is -hydrogen, -halogen, —C≡N, —OH, —NHR^{a}, —NR^{a}R^{b}, -straight or branched-(C₁₋₆)-alkyl or -straight or branched-(C₁₋₆)-haloalkyl, —(CF₂)ₙ(CH₂)ₒOH, —OR^{c} or —S(O)ₚR^{d};

where:
R⁴ is -hydrogen, -halogen, —C≡N, —OH, —NHR^{a}, —NR^{a}R^{b}, -straight or branched-(C₁₋₆)-alkyl or -straight or branched-(C₁₋₆)-haloalkyl, —(CF₂)ₙ(CH₂)ₒOH, —OR^{c} or —S(O)ₚR^{d};
where:
   R⁴ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R¹, R² or R³ optionally is substituted with -hydrogen, -halogen, —C≡N, NHR^{a}, —NR^{a}R^{b}, -straight or branched-(C₁₋₆)-alkyl, -straight or branched-(C₁₋₆)-haloalkyl or —OR^{c};

R⁵ is an unsaturated or saturated carbocyclic ring, —CH₂-unsaturated carbocyclic ring, unsaturated or saturated heterocyclic or heteroaryl ring;

where:
the unsaturated or saturated heterocyclic ring of R⁵ or the heteroaryl ring of R⁵, respectively, contains at least one heteroatom selected from nitrogen, oxygen or sulfur;
where:
   R⁵ optionally is substituted with hydrogen, halogen, —C≡N, NHR^{a}, NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;

R⁶ is -hydrogen, -straight or branched-(C₁₋₆)-alkyl or -straight or branched-(C₁₋₆)-haloalkyl;

R⁷ is

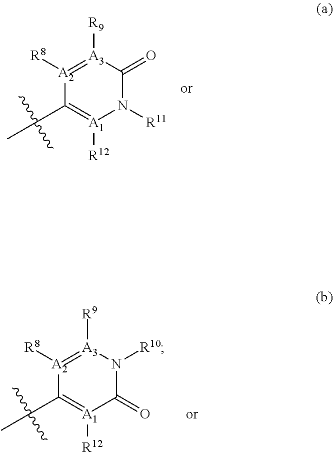

a corresponding tautomer form thereof;

where:
A₁, A₂ or A₃ is N or C;
R⁸, R⁹ or R¹² is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;
   R¹⁰ or R¹¹ is -hydrogen or -straight or branched (C₁₋₆) alkyl;
where:
   each R^{a}, R^{b} or R^{c} of R¹, R², R³, R⁴, R⁸, R⁹, R¹⁰, R¹¹ or R¹² as defined above is hydrogen, -straight or branched-(C₁₋₆)-alkyl, -straight or branched-(C₁₋₆)-haloalkyl or —(C₃₋₆)-cycloalkyl;
   where:
      R^{a} optionally further is substituted with —OH;
      R^{d} is -hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
      where:
         each R^{a} or R^{b} of NHR^{a} or NR^{a}R^{b} as defined in R^{d} is -hydrogen, -straight or branched-(C₁₋₆)-alkyl, -straight or branched-(C₁₋₆)-haloalkyl or —(C₃₋₆)-cycloalkyl;

n, o or p is 0 or an integer from 1 to 5; or

a pharmaceutically acceptable salt and/or a corresponding tautomer form thereof.

In another aspect, the present invention relates to a compound of Formula (IA):

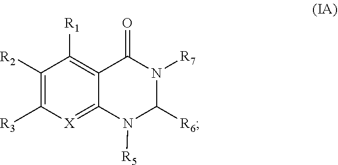

where:

X is N or C—R⁴;

R¹ is -hydrogen, -halogen, -straight or branched-(C₁₋₆)-alkyl or -straight or branched-(C₁₋₆)-haloalkyl;

R² or R³ is -hydrogen, -halogen, —C≡N, —OH, —NHR^{a}, —NR^{a}R^{b}, -straight or branched-(C₁₋₆)-alkyl, —(CF₂)ₙ(CH₂)ₒOH, —OR^{c} or —S(O)ₚR^{d};

where:
R⁴ is -hydrogen, -halogen, —C≡N, —OH, —NHR^{a}, —NR^{a}R^{b}, -straight or branched-(C₁₋₆)-alkyl or -straight or branched-(C₁₋₆)-haloalkyl, —(CF₂)ₙ(CH₂)ₒOH, —OR^{c} or —S(O)ₚR^{d};
where:
   R⁴ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};
R¹, R² or R³ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R⁵ is an unsaturated or saturated carbocyclic ring, —CH₂-unsaturated carbocyclic ring, unsaturated or saturated heterocyclic or heteroaryl ring;

where:
the unsaturated or saturated heterocyclic ring of R⁵ or the heteroaryl ring of R⁵, respectively, contains at least one heteroatom selected from nitrogen, oxygen or sulfur;
where:
   R⁵ optionally is substituted with -hydrogen, halogen, —C≡N, —NHR^{a}, —NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;

R⁶ is -hydrogen, -straight or branched-(C₁₋₆)-alkyl or -straight or branched-(C₁₋₆)-haloalkyl;

R⁷ is

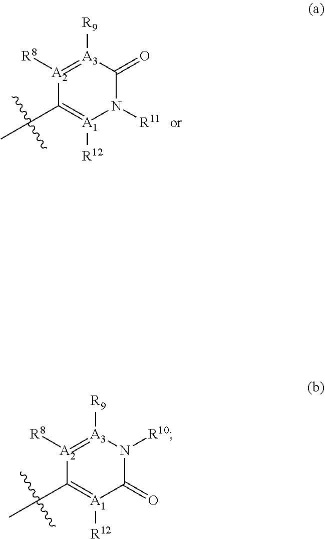

or
a corresponding tautomer form thereof

where:
A₁, A₂ or A₃ is N or C;
R⁸, R⁹ or R¹² is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;
   R¹⁰ or R¹¹ is -hydrogen, -straight or branched (C₁₋₆) alkyl;
where:
   R^{a}, R^{b} or R^{c} of R¹, R², R³, R⁴, R⁸, R⁹, R¹⁰, R¹¹ or R¹² as defined above is hydrogen, -straight or branched-(C₁₋₆)-alkyl, -straight or branched-(C₁₋₆)-haloalkyl or —(C₃₋₆)-cycloalkyl;
   where:
      R^{a} optionally further is substituted with —OH;
      R^{d} is -hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
      where:
         each R^{a} or R^{b} of NHR^{a} or NR^{a}R^{b} as defined for R^{d} respectively, is -hydrogen, -straight or branched-(C₁₋₆)-alkyl, -straight or branched-(C₁₋₆)-haloalkyl or —(C₃₋₆)-cycloalkyl;

n, o or p is 0 or an integer from 1 to 5; or

a pharmaceutically acceptable salt and/or a corresponding tautomer form thereof.

In another aspect, the present invention relates to a compound or a pharmaceutically acceptable salt thereof, where X is N or X is C—R⁴.

In another aspect, the present invention relates to a compound according to Formula (I) or Formula (IA), where X is C—R⁴.

where:
R⁴ is -hydrogen, -halogen, —C≡N, —OH, —NHR^{a}, —NR^{a}R^{b}, -straight or branched-(C₁₋₆)-alkyl or -straight or branched-(C₁₋₆)-haloalkyl, —(CF₂)ₙ(CH₂)ₒOH, —OR^{c} or —S(O)ₚR^{d};
where:
   R⁴ optionally is substituted with -hydrogen, -halogen, —C≡N, NR^{a}R^{b}, -straight or branched-(C₁₋₆)-alkyl, -straight or branched-(C₁₋₆)-haloalkyl or —OR^{c};
   where:
      R^{a}, R^{b} or R^{c} is -hydrogen, -straight or branched-(C₁₋₆)-alkyl, -straight or branched-(C₁₋₆)-haloalkyl or —(C₃₋₆)-cycloalkyl;
      where:
         R^{a} optionally further is substituted with —OH;
      R^{d} is -hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
      where:
         R^{a} or R^{b} of NHR^{a} or NR^{a}R^{b} as defined for R^{d}, respectively, is -hydrogen, -straight or branched-(C₁₋₆)-alkyl, -straight or branched-(C₁₋₆)-haloalkyl or —(C₃₋₆)-cycloalkyl.

In another aspect, the present invention relates to any compound or a pharmaceutically acceptable salt thereof of the present invention, where halogen is selected from bromo, chloro, fluoro or iodo.

In another aspect, the present invention relates to a compound which is:

| Name | Structure |
|---|---|
| 1-(4-fluoro-2-methylphenyl)-3-(2- methyl-6-oxo-1,6-dihydropyridin-3-yl)- 7-(trifluoromethyl)-2,3- dihydroquinazolin-4(1H)-one | 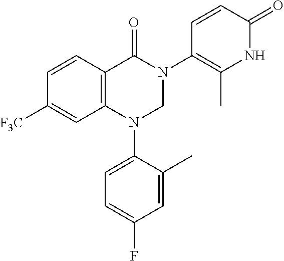 [SMILES: Cc1cc(F)ccc1N1CN(c2ccc(=O)[nH]c2C)C(=O)c2ccc(C(F)(F)F)cc21] |
| 1-(4-fluoro-2-methylphenyl)-3- (-6-oxo-1,6-dihydropyridin-3-yl)- 7-(trifluoromethyl)-2,3- dihydroquinazolin-4(1H)-one | 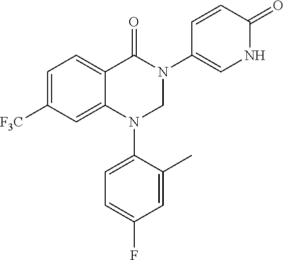 [SMILES: Cc1cc(F)ccc1N1CN(c2ccc(=O)[nH]c2)C(=O)c2ccc(C(F)(F)F)cc21] |
| 1-cyclohexyl-3-(6-oxo-1,6- dihydropyridin-3-yl)-7-(trifluoromethyl)- 2,3-dihydroquinazolin-4(1H)-one | 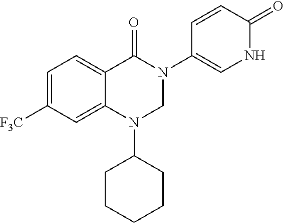 [SMILES: O=C1c2ccc(C(F)(F)F)cc2N(C2CCCCC2)CN1c1ccc(=O)[nH]c1] |
| 1-(4-fluoro-2,6-di-methylphenyl)-3- (-6-oxo-1,6-dihydropyridin-3-yl)- 7-(trifluoromethyl)-2,3- dihydroquinazolin-4(1H)-one | 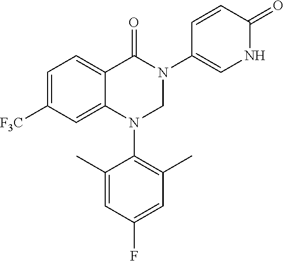 [SMILES: Cc1cc(F)cc(C)c1N1CN(c2ccc(=O)[nH]c2)C(=O)c2ccc(C(F)(F)F)cc21] |
| 1-(4-fluoro-2-methylphenyl)-2-methyl- 3-(-6-oxo-1,6-dihydropyridin-3-yl)- 7-(trifluoromethyl)-2,3- dihydroquinazolin-4(1H)-one | 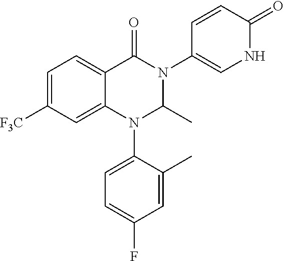 [SMILES: Cc1cc(F)ccc1N1c2cc(C(F)(F)F)ccc2C(=O)N(c2ccc(=O)[nH]c2)C1C] |
| 1-(2-chloro-4-fluorophenyl)-3-(6-oxo- 1,6-dihydropyridin-3-yl)-7- (trifluoromethyl)-2,3-dihydroquinazolin- 4(1H)-one | 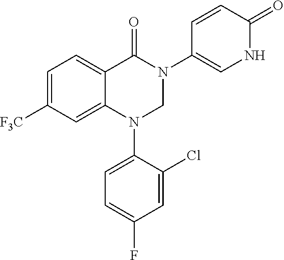 [SMILES: O=C1c2ccc(C(F)(F)F)cc2N(c2ccc(F)cc2Cl)CN1c1ccc(=O)[nH]c1] |
| 1-(4-fluoro-2-(2- hydroxyethoxy)phenyl)-3-(6-oxo-1,6- dihydropyridin-3-yl)-7-(trifluoromethyl)- 2,3-dihydroquinazolin-4(1H)-one | 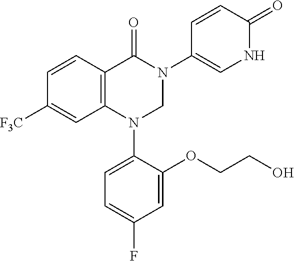 [SMILES: O=C1c2ccc(C(F)(F)F)cc2N(c2ccc(F)cc2OCCO)CN1c1ccc(=O)[nH]c1] |
| 1-(2,4-difluorophenyl)-3-(6-oxo-1,6- dihydropyridin-3-yl)-7-(trifluoromethyl)- 2,3-dihydroquinazolin-4(1H)-one | 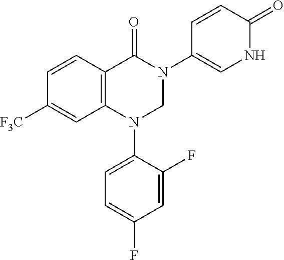 [SMILES: O=C1c2ccc(C(F)(F)F)cc2N(c2ccc(F)cc2F)CN1c1ccc(=O)[nH]c1] |
| 1-(2-ethyl-4-fluorophenyl)-3-(6-oxo-1,6- dihydropyridin-3-yl)-7-(trifluoromethyl)- 2,3-dihydroquinazolin-4(1H)-one | 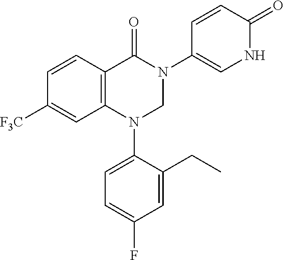 [SMILES: CCc1cc(F)ccc1N1CN(c2ccc(=O)[nH]c2)C(=O)c2ccc(C(F)(F)F)cc21] |
| 8-chloro-1-(4-fluoro-2-methylphenyl)-3- (6-oxo-1,6-dihydropyridin-3-yl)-7- (trifluoromethyl)-2,3-dihydroquinazolin- 4(1H)-one | 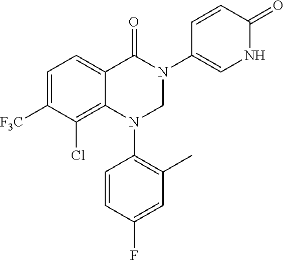 [SMILES: Cc1cc(F)ccc1N1CN(c2ccc(=O)[nH]c2)C(=O)c2ccc(C(F)(F)F)c(Cl)c21] |
| 1-(4-fluoro-2-methylphenyl)-8-methyl- 3-(6-oxo-1,6-dihydropyridin-3-yl)-7- (trifluoromethyl)-2,3-dihydroquinazolin- 4(1H)-one | 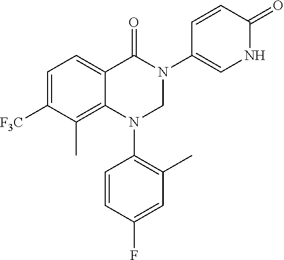 [SMILES: Cc1cc(F)ccc1N1CN(c2ccc(=O)[nH]c2)C(=O)c2ccc(C(F)(F)F)c(C)c21] |
| 3-(2-methyl-6-oxo-1,6-dihydropyridin-3- yl)-1-(2-methylpyridin-3-yl)-7- (trifluoromethyl)-2,3-dihydroquinazolin- 4(1H)-one | 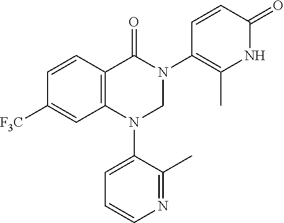 [SMILES: Cc1ncccc1N1CN(c2ccc(=O)[nH]c2C)C(=O)c2ccc(C(F)(F)F)cc21] |
| 1-(4-fluoro-2-isopropylphenyl)-3-(2- methyl-6-oxo-1,6-dihydropyridin-3-yl)- 7-(trifluoromethyl)-2,3- dihydroquinazolin-4(1H)-one | 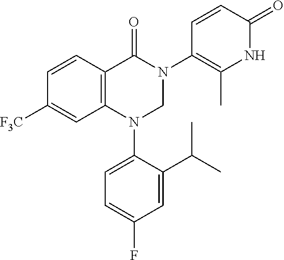 [SMILES: Cc1[nH]c(=O)ccc1N1CN(c2ccc(F)cc2C(C)C)c2cc(C(F)(F)F)ccc2C1=O] |
| 6-chloro-1-(4-fluoro-2-methylphenyl)-3- (2-methyl-6-oxo-1,6-dihydropyridin-3- yl)-2,3-dihydroquinazolin-4(1H)-one | 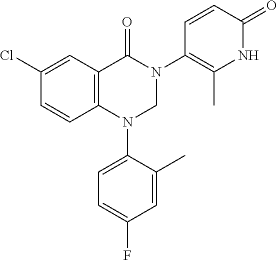 [SMILES: Cc1cc(F)ccc1N1CN(c2ccc(=O)[nH]c2C)C(=O)c2cc(Cl)ccc21] |
| 1-(4-fluorophenyl)-3-(2-methyl-6-oxo- 1,6-dihydropyridin-3-yl)-2,3- dihydroquinazolin-4(1H)-one | 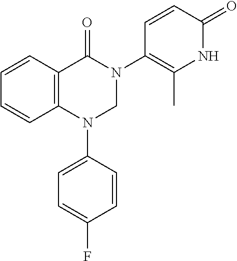 [SMILES: Cc1[nH]c(=O)ccc1N1CN(c2ccc(F)cc2)c2ccccc2C1=O] |
| 3-(2-methyl-6-oxo-1,6-dihydropyridin-3- yl)-1-(o-tolyl)-2,3-dihydroquinazolin- 4(1H)-one | 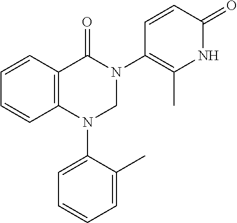 [SMILES: Cc1ccccc1N1CN(c2ccc(=O)[nH]c2C)C(=O)c2ccccc21] |
| 1-(4-fluoro-2-methylphenyl)-6-methyl- 3-(2-methyl-6-oxo-1,6-dihydropyridin-3- yl)-2,3-dihydroquinazolin-4(1H)-one | 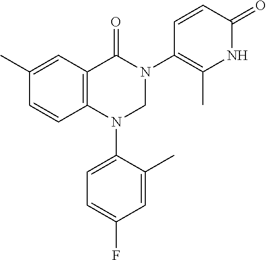 [SMILES: Cc1ccc2c(c1)C(=O)N(c1ccc(=O)[nH]c1C)CN2c1ccc(F)cc1C] |
| 1-(4-fluoro-2-methylphenyl)-3-(2- methyl-6-oxo-1,6-dihydropyridin-3-yl)- 2,3-dihydroquinazolin-4(1H)-one | 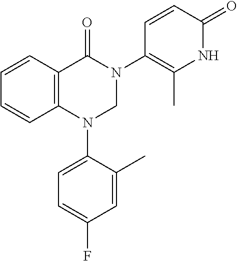 [SMILES: Cc1cc(F)ccc1N1CN(c2ccc(=O)[nH]c2C)C(=O)c2ccccc21] |
| 1-(4-fluoro-2-methylphenyl)-3-(6- methyl-2-oxo-1,2-dihydropyridin-4-yl)- 7-(trifluoromethyl)-2,3- dihydroquinazolin-4(1H)-one | 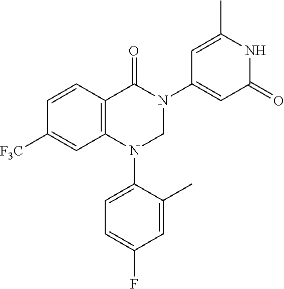 [SMILES: Cc1cc(N2CN(c3ccc(F)cc3C)c3cc(C(F)(F)F)ccc3C2=O)cc(=O)[nH]1] |
| 1-(4-fluoro-2-methylphenyl)-3-(5- methyl-2-oxo-1,2-dihydropyridin-4-yl)- 7-(trifluoromethyl)-2,3- dihydroquinazolin-4(1H)-one | 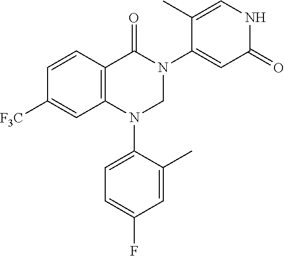 [SMILES: Cc1c[nH]c(=O)cc1N1CN(c2ccc(F)cc2C)c2cc(C(F)(F)F)ccc2C1=O] |
| 1-(4-fluoro-2-methylphenyl)-3-(3- methyl-2-oxo-1,2-dihydropyridin-4-yl)- 7-(trifluoromethyl)-2,3- dihydroquinazolin-4(1H)-one | 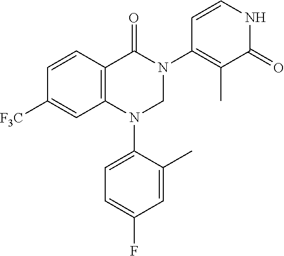 [SMILES: Cc1cc(F)ccc1N1CN(c2cc[nH]c(=O)c2C)C(=O)c2ccc(C(F)(F)F)cc21] |
| 1-(4-fluorophenyl)-3-(6-oxo-1,6- dihydropyridin-3-yl)-7-(trifluoromethyl)- 2,3-dihydroquinazolin-4(1H)-one | 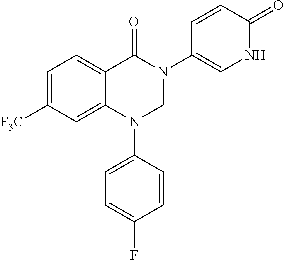 [SMILES: O=C1c2ccc(C(F)(F)F)cc2N(c2ccc(F)cc2)CN1c1ccc(=O)[nH]c1] |
| 1-(4-fluoro-2-methylphenyl)-3-(2-oxo- 1,2-dihydropyrimidin-5-yl)-7- (trifluoromethyl)-2,3-dihydroquinazolin- 4(1H)-one | 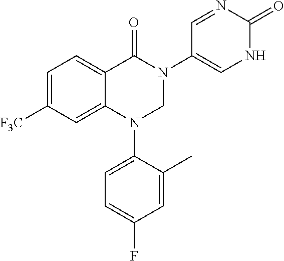 [SMILES: Cc1cc(F)ccc1N1CN(c2cnc(=O)[nH]c2)C(=O)c2ccc(C(F)(F)F)cc21] |
| 1-(4-bromo-2-methylphenyl)-3-(6-oxo- 1,6-dihydropyridin-3-yl)-7- (trifluoromethyl)-2,3-dihydroquinazolin- 4(1H)-one | 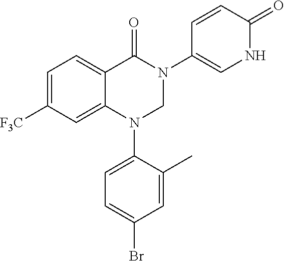 [SMILES: Cc1cc(Br)ccc1N1CN(c2ccc(=O)[nH]c2)C(=O)c2ccc(C(F)(F)F)cc21] |
| 1-(4-fluorophenyl)-3-(2-methyl-6-oxo- 1,6-dihydropyridin-3-yl)-7- (trifluoromethyl)-2,3-dihydroquinazolin- 4(1H)-one | 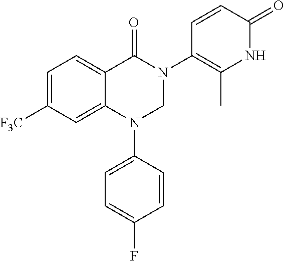 [SMILES: Cc1[nH]c(=O)ccc1N1CN(c2ccc(F)cc2)c2cc(C(F)(F)F)ccc2C1=O] |
| 3-methyl-4-(3-(2-methyl-6-oxo-1,6- dihydropyridin-3-yl)-4-oxo-7- (trifluoromethyl)-3,4-dihydroquinazolin- 1(2H)-yl)benzonitrile | 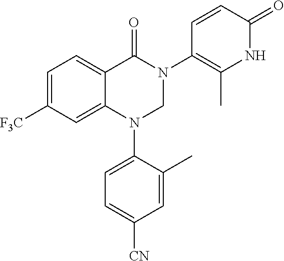 [SMILES: Cc1cc(C#N)ccc1N1CN(c2ccc(=O)[nH]c2C)C(=O)c2ccc(C(F)(F)F)cc21] |
| 1-(4-fluoro-2-methylphenyl)-3-(2- methyl-6-oxo-1,6-dihydropyridin-3-yl)- 7-(trifluoromethyl)-2,3- dihydropyrido[2,3-d]pyrimidin-4(1H)- one | 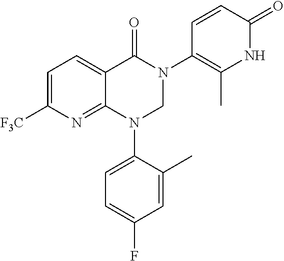 [SMILES: Cc1cc(F)ccc1N1CN(c2ccc(=O)[nH]c2C)C(=O)c2ccc(C(F)(F)F)nc21] |
| 3-(2-ethyl-6-oxo-1,6-dihydropyridin-3- yl)-1-(4-fluoro-2-methylphenyl)-7- (trifluoromethyl)-2,3-dihydroquinazolin- 4(1H)-one | 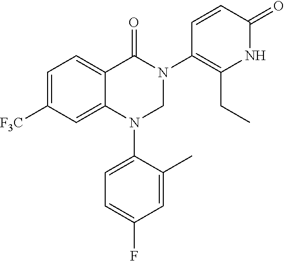 [SMILES: CCc1[nH]c(=O)ccc1N1CN(c2ccc(F)cc2C)c2cc(C(F)(F)F)ccc2C1=O] |
| 3-(2-chloro-6-oxo-1,6-dihydropyridin-3- yl)-1-(4-fluoro-2-methylphenyl)-7- (trifluoromethyl)-2,3-dihydroquinazolin- 4(1H)-one | 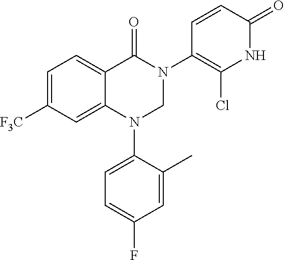 [SMILES: Cc1cc(F)ccc1N1CN(c2ccc(=O)[nH]c2Cl)C(=O)c2ccc(C(F)(F)F)cc21] |
| 7-bromo-1-(4-fluoro-2-methylphenyl)-3- (2-methyl-6-oxo-1,6-dihydropyridin-3- yl)-2,3-dihydroquinazolin-4(1H)-one | 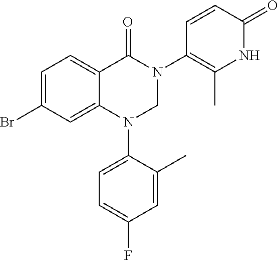 [SMILES: Cc1cc(F)ccc1N1CN(c2ccc(=O)[nH]c2C)C(=O)c2ccc(Br)cc21] |
| 1-(4-fluoro-2-methylphenyl)-3-(2- methyl-6-oxo-1,6-dihydropyridin-3-yl)- 4-oxo-1,2,3,4-tetrahydroquinazoline-7- carbonitrile | 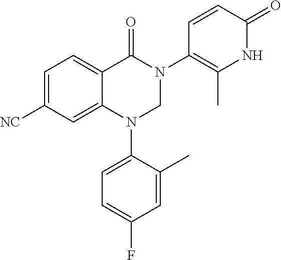 [SMILES: Cc1cc(F)ccc1N1CN(c2ccc(=O)[nH]c2C)C(=O)c2ccc(C#N)cc21] |
| 3-(1-(4-fluoro-2-methylphenyl)-4-oxo-7- (trifluoromethyl)-1,4-dihydroquinazolin- 3(2H)-yl)-6-oxo-1,6-dihydropyridine-2- carbonitrile | 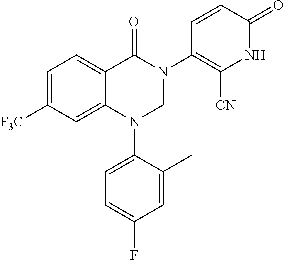 [SMILES: Cc1cc(F)ccc1N1CN(c2ccc(=O)[nH]c2C#N)C(=O)c2ccc(C(F)(F)F)cc21] |
| 1-(2,4-difluorophenyl)-3-(2-methyl-6- oxo-1,6-dihydropyridin-3-yl)-7- (trifluoromethyl)-2,3-dihydroquinazolin- 4(1H)-one | 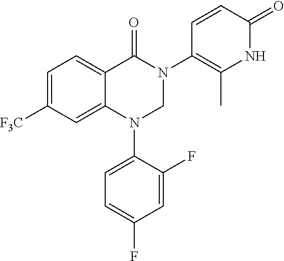 [SMILES: Cc1[nH]c(=O)ccc1N1CN(c2ccc(F)cc2F)c2cc(C(F)(F)F)ccc2C1=O] |
| 1-(4-ethoxyphenyl)-3-(2-methyl-6-oxo- 1,6-dihydropyridin-3-yl)-7- (trifluoromethyl)-2,3-dihydroquinazolin- 4(1H)-one | 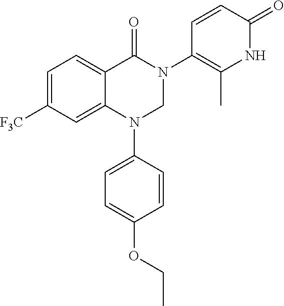 [SMILES: CCOc1ccc(N2CN(c3ccc(=O)[nH]c3C)C(=O)c3ccc(C(F)(F)F)cc32)cc1] |
| 1-(4-fluoro-2-methylphenyl)-3-(2-oxo- 1,2-dihydropyridin-4-yl)-7- (trifluoromethyl)-2,3-dihydroquinazolin- 4(1H)-one | 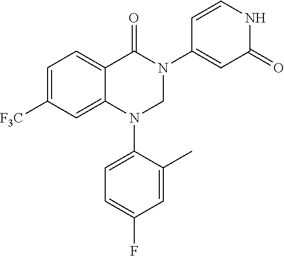 [SMILES: Cc1cc(F)ccc1N1CN(c2cc[nH]c(=O)c2)C(=O)c2ccc(C(F)(F)F)cc21] |
| 1-(4-fluoro-2-methylphenyl)-3-(6-oxo- 1,6-dihydropyridin-3-yl)-6- (trifluoromethyl)-2,3-dihydroquinazolin- 4(1H)-one | 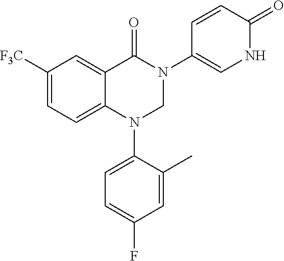 [SMILES: Cc1cc(F)ccc1N1CN(c2ccc(=O)[nH]c2)C(=O)c2cc(C(F)(F)F)ccc21] |
| 1-(4-fluoro-2-methylphenyl)-3-(6-oxo- 1,6-dihydropyridin-3-yl)-7- (trifluoromethyl)-2,3-dihydroquinazolin- 4(1H)-one | 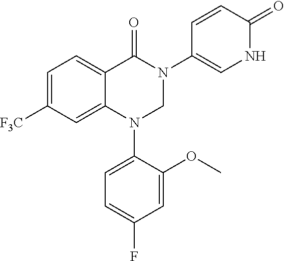 [SMILES: COc1cc(F)ccc1N1CN(c2ccc(=O)[nH]c2)C(=O)c2ccc(C(F)(F)F)cc21] |
| 1-(4-fluoro-2-methylphenyl)-3-(6-oxo- 1,6-dihydropyridin-3-yl)-5- (trifluoromethyl)-2,3-dihydroquinazolin- 4(1H)-one | 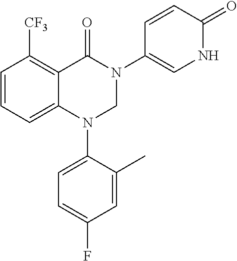 [SMILES: Cc1cc(F)ccc1N1CN(c2ccc(=O)[nH]c2)C(=O)c2c1cccc2C(F)(F)F] |
| 1-(4-fluoro-2-methylphenyl)-3-(5- methyl-6-oxo-1,6-dihydropyridin-3-yl)- 7-(trifluoromethyl)-2,3- dihydroquinazolin-4(1H)-one | 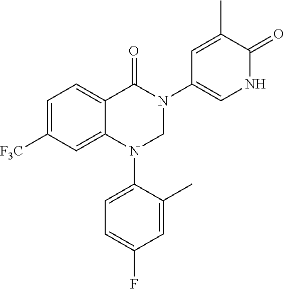 [SMILES: Cc1cc(F)ccc1N1CN(c2c[nH]c(=O)c(C)c2)C(=O)c2ccc(C(F)(F)F)cc21] |
| 1-(4-fluoro-2-methylphenyl)-3-(6-oxo- 1,6-dihydropyridazin-3-yl)-7- (trifluoromethyl)-2,3-dihydroquinazolin- 4(1H)-one | 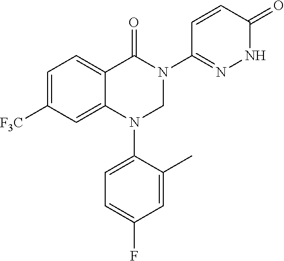 [SMILES: Cc1cc(F)ccc1N1CN(c2ccc(=O)[nH]n2)C(=O)c2ccc(C(F)(F)F)cc21] |
| 1-(4-fluorobenzyl)-3-(6-oxo-1,6- dihydropyridin-3-yl)-7-(trifluoromethyl)- 2,3-dihydroquinazolin-4(1H)-one | 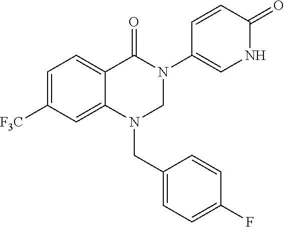 [SMILES: O=C1c2ccc(C(F)(F)F)cc2N(Cc2ccc(F)cc2)CN1c1ccc(=O)[nH]c1] |
| 1-(4-fluoro-2-methylphenyl)-3-(4- methyl-6-oxo-1,6-dihydropyridin-3-yl)- 7-(trifluoromethyl)-2,3- dihydroquinazolin-4(1H)-one | 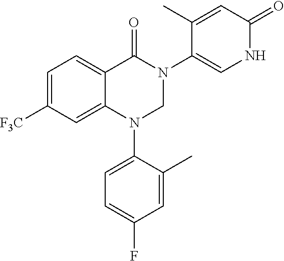 [SMILES: Cc1cc(=O)[nH]cc1N1CN(c2ccc(F)cc2C)c2cc(C(F)(F)F)ccc2C1=O] |
| 1-(4-fluoro-2-methylphenyl)-4-oxo-3-(6- oxo-1,6-dihydropyridin-3-yl)-1,2,3,4- tetrahydroquinazoline-7-carbonitrile | 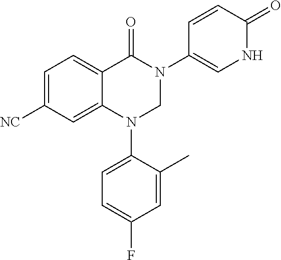 [SMILES: Cc1cc(F)ccc1N1CN(c2ccc(=O)[nH]c2)C(=O)c2ccc(C#N)cc21] |
| 1-(4-fluoro-2-methoxyphenyl)-3-(6-oxo- 1,6-dihydropyridin-3-yl)-5- (trifluoromethyl)-2,3-dihydroquinazolin- 4(1H)-one | 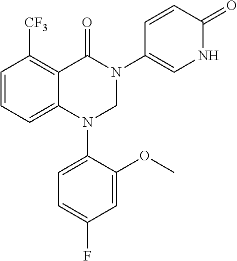 [SMILES: COc1cc(F)ccc1N1CN(c2ccc(=O)[nH]c2)C(=O)c2c1cccc2C(F)(F)F] |
| 1-(4-fluoro-2-methylphenyl)-3-(1- methyl-6-oxo-1,6-dihydropyridin-3-yl)- 7-(trifluoromethyl)-2,3- dihydroquinazolin-4(1H)-one | 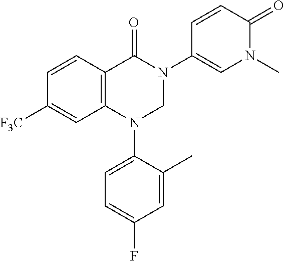 [SMILES: Cc1cc(F)ccc1N1CN(c2ccc(=O)n(C)c2)C(=O)c2ccc(C(F)(F)F)cc21] |
| 1-(4-fluoro-2-methylphenyl)-3-(2- methyl-6-oxo-1,6-dihydropyridin-3-yl)- 7-(trifluoromethoxy)-2,3- dihydroquinazolin-4(1H)-one | 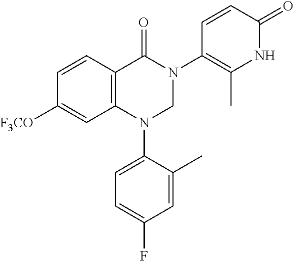 [SMILES: Cc1cc(F)ccc1N1CN(c2ccc(=O)[nH]c2C)C(=O)c2ccc(OC(F)(F)F)cc21] |
| 1-(4-fluoro-2-methylphenyl)-7-methyl- 3-(2-methyl-6-oxo-1,6-dihydropyridin-3- yl)-2,3-dihydroquinazolin-4(1H)-one | 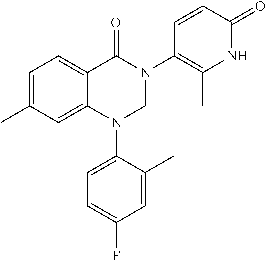 [SMILES: Cc1ccc2c(c1)N(c1ccc(F)cc1C)CN(c1ccc(=O)[nH]c1C)C2=O] |
| 1-(4-fluoro-2-methylphenyl)-3-(2- methyl-6-oxo-1,6-dihydropyridin-3- 6-(trifluoromethyl)-2,3- dihydroquinazolin-4(1H)-one | 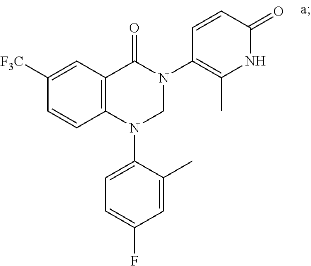 [SMILES: Cc1cc(F)ccc1N1CN(c2ccc(=O)[nH]c2C)C(=O)c2cc(C(F)(F)F)ccc21] |
| 1-(4-fluoro-2-methylphenyl)-3-(3- methyl-5-oxo-4,5-dihydropyrazin-2-yl)- 7-(trifluoromethyl)-2,3- dihydroquinazolin-4(1H)-one | 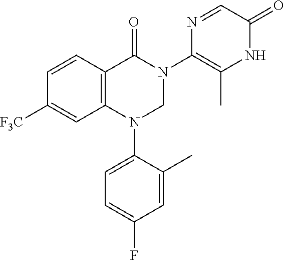 [SMILES: Cc1cc(F)ccc1N1CN(c2ncc(=O)[nH]c2C)C(=O)c2ccc(C(F)(F)F)cc21] |

or pharmaceutically acceptable salt and/or a corresponding tautomer form thereof.

In another aspect, the present invention relates to a compound which is:

| Name | Structure |
|---|---|
| 1-(4-fluoro-2-methylphenyl)-3-(2-methyl- 6-oxo-1,6-dihydropyridin-3-yl)-7- (trifluoromethyl)-2,3-dihydroquinazolin- 4(1H)-one | 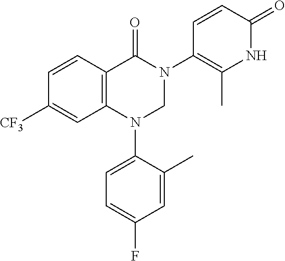 [SMILES: Cc1cc(F)ccc1N1CN(c2ccc(=O)[nH]c2C)C(=O)c2ccc(C(F)(F)F)cc21] |
| 6-chloro-1-(4-fluoro-2-methylphenyl)-3- (2-methyl-6-oxo-1,6-dihydropyridin-3- yl)-2,3-dihydroquinazolin-4(1H)-one | 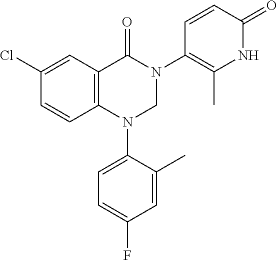 [SMILES: Cc1cc(F)ccc1N1CN(c2ccc(=O)[nH]c2C)C(=O)c2cc(Cl)ccc21] |
| 1-(4-fluoro-2-methylphenyl)-3-(2-methyl- 6-oxo-1,6-dihydropyridin-3-y1)-4-oxo- 1,2,3,4-tetrahydroquinazoline-7- carbonitrile | 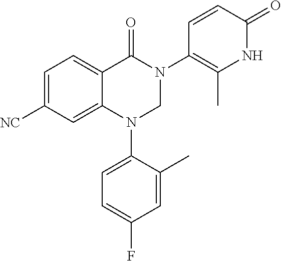 [SMILES: Cc1cc(F)ccc1N1CN(c2ccc(=O)[nH]c2C)C(=O)c2ccc(C#N)cc21] |
| 1-(4-fluoro-2-methylphenyl)-3-(2-methyl- 6-oxo-1,6-dihydropyridin-3-yl)-7- (trifluoromethoxy)-2,3- dihydroquinazolin-4(1H)-one | 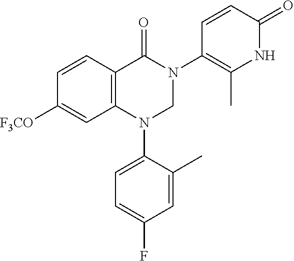 [SMILES: Cc1cc(F)ccc1N1CN(c2ccc(=O)[nH]c2C)C(=O)c2ccc(OC(F)(F)F)cc21] |
| 1-(4-fluoro-2-methylphenyl)-3-(2-methyl- 6-oxo-1,6-dihydropyridin-3-y1)-6- (trifluoromethyl)-2,3-dihydroquinazolin- 4(1H)-one | 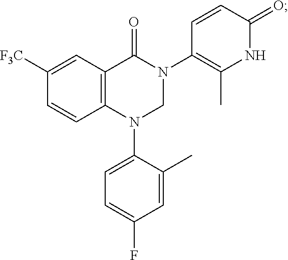 [SMILES: Cc1cc(F)ccc1N1CN(c2ccc(=O)[nH]c2C)C(=O)c2cc(C(F)(F)F)ccc21] |

or
a pharmaceutically acceptable salt and/or a corresponding tautomer form thereof.

In another aspect, the present invention relates to a compound of Formula (IB):

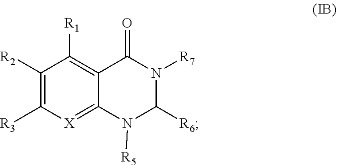

where:

X is N or C—R⁴;

R¹ is -hydrogen, -halogen, -straight or branched-(C₁₋₆)-alkyl or -straight or branched-(C₁₋₆)-haloalkyl;

R² or R³ is -hydrogen, -halogen, —C≡N, —OH, —NHR^{a}, —NR^{a}R^{b}, -straight or branched-(C₁₋₆)-alkyl, —(CF₂)ₙ(CH₂)ₒOH, —OR^{c} or —S(O)ₚR^{d};

where:
R⁴ is -hydrogen, -halogen, -straight or branched-(C₁₋₆)-alkyl or -straight or branched-(C₁₋₆)-haloalkyl;
R¹, R² or R³ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};
where:
   R⁴ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R⁵ is an unsaturated or saturated carbocyclic ring, unsaturated or saturated heterocyclic or heteroaryl ring;

where:
the unsaturated or saturated heterocyclic ring of R⁵ or the heteroaryl ring of R⁵, respectively, contains at least one heteroatom selected from nitrogen, oxygen or sulfur;
where:
   R⁵ optionally is substituted with -hydrogen, halogen, —C≡N, —NHR^{a}, —NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;

R⁶ is -hydrogen, -straight or branched-(C₁₋₆)-alkyl or -straight or branched-(C₁₋₆)-haloalkyl;

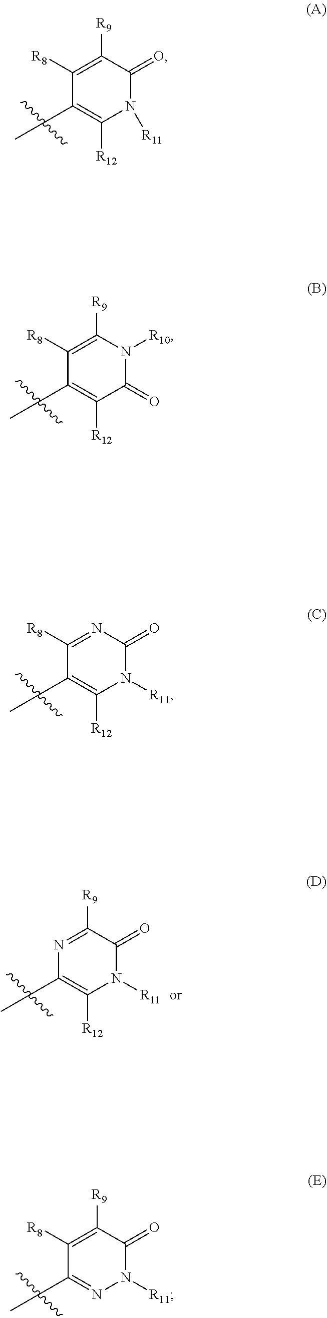

or a corresponding tautomer form thereof;

where:
R⁸, R⁹ or R¹² is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;
R¹⁰ or R¹¹ is -hydrogen or -straight or branched (C₁₋₆) alkyl;
where:
   each R^{a}, R^{b} or R^{c} of R¹, R², R³, R⁴, R⁸, R⁹, R¹⁰, R¹¹ or R¹² as defined above is hydrogen, -straight or branched-(C₁₋₆)-alkyl, -straight or branched-(C₁₋₆)-haloalkyl or —(C₃₋₆)-cycloalkyl;
where:
   R^{a} optionally further is substituted with —OH;
   R^{d} is -hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
   where:
      each R^{a} or R^{b} of NHR^{a} or NR^{a}R^{b} as defined in R^{d} respectively, is -hydrogen, -straight or branched-(C₁₋₆)-alkyl, -straight or branched-(C₁₋₆)-haloalkyl or —(C₃₋₆)-cycloalkyl;
n, o or p is 0 or an integer from 1 to 5; or
   a pharmaceutically acceptable salt and/or a corresponding tautomer form thereof.

In another aspect, the present invention relates to a compound of Formula (II):

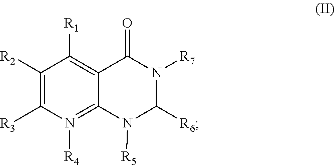

where:

R¹ or R⁴ is -hydrogen, -halogen, -straight or branched-(C₁₋₆)-alkyl or -straight or branched-(C₁₋₆)-haloalkyl;

R² or R³ is -hydrogen, -halogen, —C≡N, —OH, —NHR^{a}, —NR^{a}R^{b}, -straight or branched-(C₁₋₆)-alkyl, —(CF₂)ₙ(CH₂)ₒOH, —OR^{o} or —S(O)ₚR^{d};

where:
each R¹, R², R³ or R⁴ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R⁵ is an unsaturated or saturated carbocyclic ring, unsaturated or saturated heterocyclic or heteroaryl ring;

where:
the unsaturated or saturated heterocyclic of R⁵ or the heteroaryl ring of R⁵, respectively, contains at least one heteroatom selected from nitrogen, oxygen or sulfur;
where:
   each R⁵ optionally is substituted with hydrogen, halogen, —C≡N, —NHR^{a}, —NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;

R⁶ is -hydrogen, -straight or branched-(C₁₋₆)-alkyl or -straight or branched-(C₁₋₆)-haloalkyl;

R⁷ is

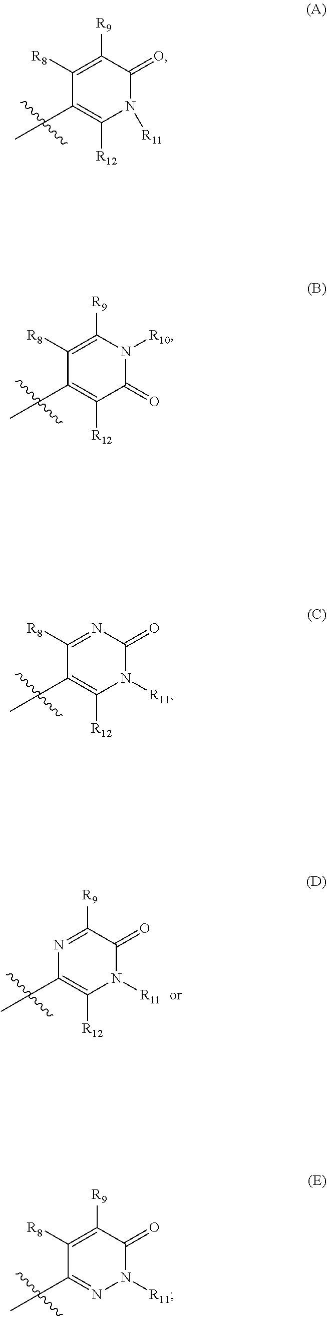

or a corresponding tautomer form thereof;

where:
each R⁸, R⁹ or R¹² is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;
each R¹⁰ or R¹¹ is -hydrogen, -straight or branched (C₁₋₆) alkyl;
where:
   for each corresponding R^{a}, R^{b} or R^{c} defined in substituent groups R¹, R², R³, R⁴, R⁶, R⁸, R⁹, R¹¹, R¹³, R¹⁴, R¹⁵, R¹⁶ or R¹⁷ above is hydrogen, -straight or branched-(C₁₋₆)-alkyl, -straight or branched-(C₁₋₆)-haloalkyl or —(C₃₋₆)-cycloalkyl;
   where:
      R^{a} optionally further is substituted with —OH;
      R^{d} is -hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
   where:
      each corresponding R^{a} or R^{b} associated with NHR^{a} or NR^{a}R^{b} as defined in R^{d} respectively, is -hydrogen, -straight or branched-(C₁₋₆)-alkyl, -straight or branched-(C₁₋₆)-haloalkyl or —(C₃₋₆)-cycloalkyl;

n, o or p is 0 or an integer from 1 to 5; or

a pharmaceutically acceptable salt and/or a corresponding tautomer form thereof.

In another aspect, the present invention relates to a compound of Formula (II):

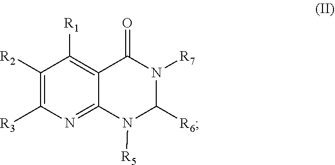

where:

R¹ or R⁴ is -hydrogen, -halogen, -straight or branched-(C₁₋₆)-alkyl or -straight or branched-(C₁₋₆)-haloalkyl;

R² or R³ is -hydrogen, -halogen, —C≡N, —OH, —NHR^{a}, —NR^{a}R^{b}, -straight or branched-(C₁₋₆)-alkyl, —(CF₂)ₙ(CH₂)ₒOH, —OR^{c} or —S(O)ₚR^{d};

where:
R¹, R², R³ or R⁴ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R⁵ is an unsaturated or saturated carbocyclic ring, unsaturated or saturated heterocyclic or heteroaryl ring;

where:
the unsaturated or saturated heterocyclic ring of R⁵ or the heteroaryl ring of R⁵, respectively, contains at least one heteroatom selected from nitrogen, oxygen or sulfur;
where:
   R⁵ optionally is substituted with hydrogen, halogen, —C≡N, —NHR^{a}, —NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;

R⁶ is -hydrogen, -straight or branched-(C₁₋₆)-alkyl or -straight or branched-(C₁₋₆)-haloalkyl;

R⁷ is

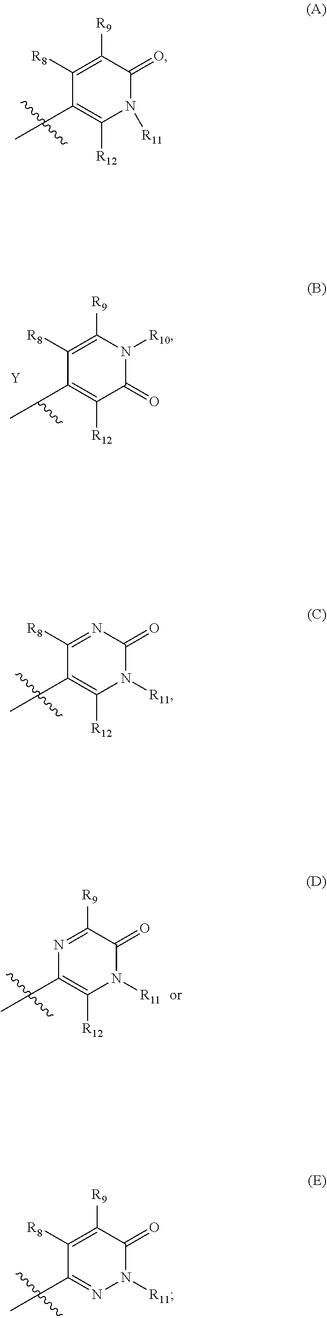

or a corresponding tautomer form thereof;

where:
R⁸, R⁹ or R¹² is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;
each R¹⁰ or R¹¹ is -hydrogen, -straight or branched (C₁₋₆) alkyl;
where:
   each R^{a}, R^{b} or R^{c} of R¹, R², R³, R⁴, R⁸, R⁹, R¹⁰, R¹¹ or R¹² as defined above is hydrogen, -straight or branched-(C₁₋₆)-alkyl, -straight or branched-(C₁₋₆)-haloalkyl or —(C₃₋₆)-cycloalkyl;
   where:
      R^{a} optionally further is substituted with —OH;
      R^{d} is -hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
   where:
      each R^{a} or R^{b} of NHR^{a} or NR^{a}R^{b} as defined in R^{d} respectively, is -hydrogen, -straight or branched-(C₁₋₆)-alkyl, -straight or branched-(C₁₋₆)-haloalkyl or —(C₃₋₆)-cycloalkyl;

n, o or p is 0 or an integer from 1 to 5; or

a pharmaceutically acceptable salt and/or a corresponding tautomer form thereof.

In another aspect, the present invention relates to a compound of Formula (IIA):

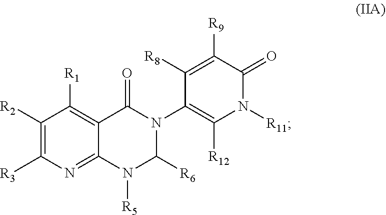

where:

R^{1′} is -hydrogen, -halogen, -straight or branched-(C₁₋₆)-alkyl or -straight or branched-(C₁₋₆)-haloalkyl;

R² or R³ is -hydrogen, -halogen, —C≡N, —OH, —NHR^{a}, —NR^{a}R^{b}, -straight or branched-(C₁₋₆)-alkyl, —(CF₂)ₙ(CH₂)ₒOH, —OR^{c} or —S(O)ₚR^{d};

where:
R¹, R² or R³ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R⁵ is an unsaturated or saturated carbocyclic ring, unsaturated or saturated heterocyclic or heteroaryl ring;
where:
   the unsaturated or saturated heterocyclic ring of R⁵ or the heteroaryl ring of R⁵, respectively, contains at least one heteroatom selected from nitrogen, oxygen or sulfur;
   where:
      R⁵ optionally is substituted with hydrogen, halogen, —C≡N, —NHR^{a}, —NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;

R⁶ is -hydrogen, -straight or branched-(C₁₋₆)-alkyl or -straight or branched-(C₁₋₆)-haloalkyl;

R⁸, R⁹ or R¹² is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;

R¹⁰ or R¹¹ is -hydrogen or -straight or branched (C₁₋₆) alkyl;
where:
   each R^{a}, R^{b} or R^{c} of R¹, R², R³, R⁴, R⁸, R⁹, R₁₀, R¹¹ or R¹² as defined above is hydrogen, -straight or branched-(C₁₋₆)-alkyl, -straight or branched-(C₁₋₆)-haloalkyl or —(C₃₋₆)-cycloalkyl;
   where:
      R^{a} optionally further is substituted with —OH;
      R^{d} is -hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
      where:
         R^{a} or R^{b} of NHR^{a} or NR^{a}R^{b} as defined for R^{d} is -hydrogen, -straight or branched-(C₁₋₆)-alkyl, -straight or branched-(C₁₋₆)-haloalkyl or —(C₃₋₆)-cycloalkyl;

n, o or p is 0 or an integer from 1 to 5; or

a pharmaceutically acceptable salt and/or a corresponding tautomer form thereof.

In another aspect, the present invention relates to a compound of Formula (IIB):

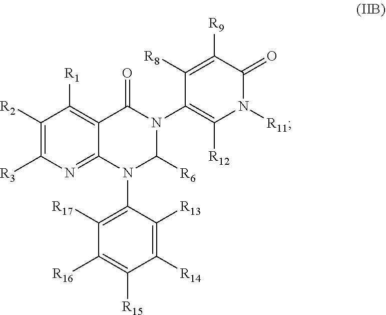

where:

R¹ is -hydrogen, -halogen, -straight or branched-(C₁₋₆)-alkyl or -straight or branched-(C₁₋₆)-haloalkyl;

R² or R³ is -hydrogen, -halogen, —C≡N, —OH, —NHR^{a}, —NR^{a}R^{b}, -straight or branched-(C₁₋₆)-alkyl, —(CF₂)ₙ(CH₂)ₒOH, —OR^{c} or —S(O)ₚR^{d};

where:
R¹, R² or R³ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R⁶ is -hydrogen, -straight or branched-(C₁₋₆)-alkyl or -straight or branched-(C₁₋₆)-haloalkyl;

R⁸, R⁹ or R¹² is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;

R¹³, R¹⁴, R¹⁵, R¹⁶ or R¹⁷ is -hydrogen, -halogen, —C≡N, —OR^{c}, -straight or branched (C₁₋₆) alkyl, -straight or branched (C₁₋₆)haloalkyl, —(C₃₋₆)-cycloalkyl, aryl or heteroaryl;

where:
R¹³, R¹⁴, R¹⁵, R¹⁶ or R¹⁷ optionally is substituted with hydrogen, halogen, —C≡N, —NHR^{a}, —NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;
where:
   each R^{a}, R^{b} or R^{c} of R¹, R², R³, R⁴, R⁸, R⁹, R¹¹, R¹³, R¹⁴, R¹⁵, R¹⁶ or R¹⁷ as defined above is hydrogen, -straight or branched-(C₁₋₆)-alkyl, -straight or branched-(C₁₋₆)-haloalkyl or —(C₃₋₆)-cycloalkyl
   where:
      R^{a} optionally further substituted with —OH;
      R^{d} is hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
      where:
         each R^{a} or R^{b} of NHR^{a} or NR^{a}R^{b} as defined in R^{d} is hydrogen, -straight or branched-(C₁₋₆)-alkyl, -straight or branched-(C₁₋₆)-haloalkyl or —(C₃₋₆)-cycloalkyl;

n, o or p is 0 or an integer from 1 to 5; or

a pharmaceutically acceptable salt and/or a corresponding tautomer form thereof.

In another aspect, the present invention relates to a compound which is 1-(4-fluoro-2-methylphenyl)-3-(2-methyl-6-oxo-1,6-dihydropyridin-3-yl)-7-(trifluoromethyl)-2,3-dihydropyrido[2,3-d]pyrimidin-4(1H)-one

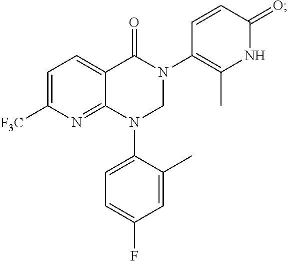 [SMILES: Cc1cc(F)ccc1N1CN(c2ccc(=O)[nH]c2C)C(=O)c2ccc(C(F)(F)F)nc21]

or
a pharmaceutically acceptable salt and/or a corresponding tautomer form thereof.

In another aspect, the present invention relates to a compound of Formula (III):

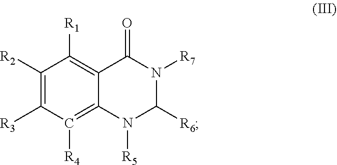

where:

R¹ or R⁴ is -hydrogen, -halogen, -straight or branched-(C₁₋₆)-alkyl or -straight or branched-(C₁₋₆)-haloalkyl;

R² or R³ is -hydrogen, -halogen, —C≡N, —OH, —NHR^{a}, —NR^{a}R^{b}, -straight or branched-(C₁₋₆)-alkyl, —(CF₂)ₙ(CH₂)ₒOH, —OR^{c} or —S(O)ₚR^{d};

where:
R¹, R², R³ or R⁴ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R⁵ is an unsaturated or saturated carbocyclic ring, —CH₂-unsaturated carbocyclic ring, unsaturated or saturated heterocyclic or heteroaryl ring;

where:
the unsaturated or saturated heterocyclic ring of R⁵ or the heteroaryl ring of R⁵, respectively, contains at least one heteroatom selected from nitrogen, oxygen or sulfur;
where:
   R⁵ optionally is substituted with hydrogen, halogen, —C≡N, —NHR^{a}, —NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;

R⁶ is -hydrogen, -straight or branched-(C₁₋₆)-alkyl or -straight or branched-(C₁₋₆)-haloalkyl;

R⁷ is

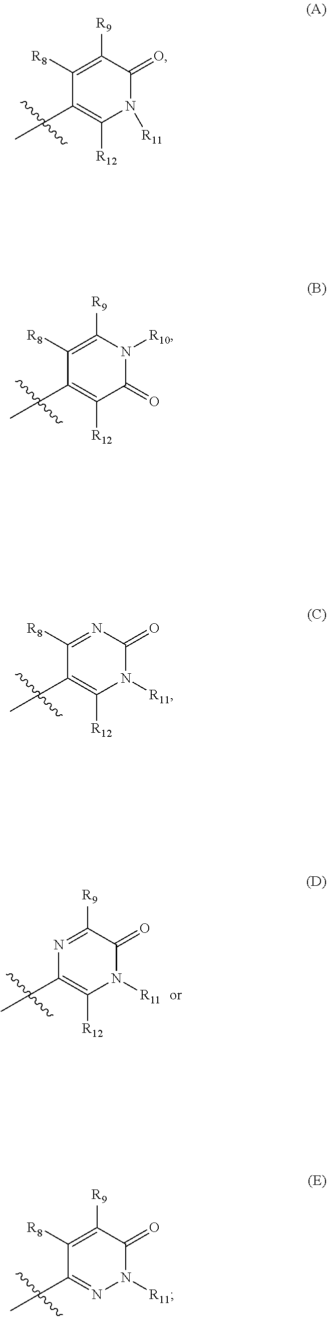

or a corresponding tautomer form thereof;

where:
R⁸, R⁹ or R¹² is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;
R¹⁰ or R¹¹ is -hydrogen, -straight or branched (C₁₋₆) alkyl;
where:
   each R^{a}, R^{b} or R^{c} of R¹, R², R³, R⁴, R⁸, R⁹, R¹⁰, R¹¹ or R¹² as defined above is hydrogen, -straight or branched-(C₁₋₆)-alkyl, -straight or branched-(C₁₋₆)-haloalkyl or —(C₃₋₆)-cycloalkyl;
   where:
      R^{a} optionally further is substituted with —OH;
      R^{d} is -hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
      where:
         each R^{a} or R^{b} of NHR^{a} or NR^{a}R^{b} as defined for R^{d} respectively, is -hydrogen, -straight or branched-(C₁₋₆)-alkyl, -straight or branched-(C₁₋₆)-haloalkyl or —(C₃₋₆)-cycloalkyl;

n, o or p is 0 or an integer from 1 to 5; or

a pharmaceutically acceptable salt and/or a corresponding tautomer form thereof.

In another aspect, the present invention relates to a compound selected from:
1-(4-fluoro-2-methylphenyl)-3-(1-methyl-6-oxo-1,6-dihydropyridin-3-yl)-7-(trifluoromethyl)-2,3-dihydro quinazolin-4(1H)-one;
1-(4-fluoro-2-methylphenyl)-3-(pyridin-3-yl)-7-(trifluoromethyl)-2,3-dihydroquinazolin-4(1H)-one;
1-(4-fluoro-2-methylphenyl)-3-(2-methoxypyrimidin-5-yl)-7-(trifluoromethyl)-2,3-dihydroquinazolin-4(1H)-one;
1-(4-fluoro-2-methylphenyl)-3-(1-methyl-1H-pyrazol-5-yl)-6-(trifluoromethyl)-2,3-dihydroquinazolin-4(1H)-one;
N-(3-(1-(4-fluoro-2-methylphenyl)-4-oxo-7-(trifluoromethyl)-1,4-dihydroquinazolin-3(2H)-yl)phenyl)acetamide;
N-(4-(1-(4-fluoro-2-methylphenyl)-4-oxo-7-(trifluoromethyl)-1,4-dihydroquinazolin-3(2H)-yl)phenyl)acetamide;
5-(6-chloro-5-fluoro-1-(4-fluoro-2-methylphenyl)-4-oxo-1,4-dihydroquinazolin-3(2H)-yl)picolinamide;
4-(6-chloro-5-fluoro-1-(4-fluoro-2-methylphenyl)-4-oxo-1,4-dihydroquinazolin-3(2H)-yl)picolinamide;
1-(2-bromo-4-fluorophenyl)-3-(6-methoxy-2-methylpyridin-3-yl)-6-(trifluoromethyl)-2,3-dihydroquinazolin-4(1H)-one;
1-(4-fluoro-2-methylphenyl)-3-(6-methoxypyridin-3-yl)-7-(trifluoromethyl)-2,3-dihydroquinazolin-4(1H)-one;
6-chloro-7-fluoro-1-(2-methyl-4-(trifluoromethoxy)phenyl)-3-(3-methylpyridin-4-yl)-2,3-dihydroquinazolin-4(1H)-one;
6-chloro-1-(2-ethyl-4-fluorophenyl)-7-fluoro-3-(3-methylpyridin-4-yl)-2,3-dihydroquinazolin-4(1H)-one;
1-(4-fluoro-2-methylphenyl)-3-(4-(methylsulfonyl)phenyl)-7-(trifluoromethyl)-2,3-dihydroquinazolin-4(1H)-one;
1-(4-fluoro-2-methylphenyl)-3-(3-(methylsulfonyl)phenyl)-7-(trifluoromethyl)-2,3-dihydroquinazolin-4(1H)-one;
3-(1,1-dioxidotetrahydrothiophen-3-yl)-1-(4-fluoro-2-methylphenyl)-6-(trifluoromethyl)-2,3-dihydroquinazolin-4(1H)-one;
1-(4-fluoro-2-methylphenyl)-3-(3-methylpyridin-4-yl)-6-(trifluoromethyl)-2,3-dihydroquinazolin-4(1H)-one;
3-(1,1-dioxidotetrahydro-2H-thiopyran-4-yl)-1-(4-fluoro-2-methylphenyl)-7-(trifluoromethyl)-2,3-dihydroquinazolin-4(1H)-one;
1-(4-fluoro-2-methylphenyl)-3-(1H-pyrazol-4-yl)-6-(trifluoromethyl)-2,3-dihydroquinazolin-4(1H)-one;
1-(4-fluoro-2-methylphenyl)-3-(6-methoxy-2-methylpyridin-3-yl)-6-(trifluoromethyl)-2,3-dihydroquinazolin-4(1H)-one;
1-(4-fluoro-2-methylphenyl)-3-(4-methyl-6-oxo-1,6-dihydropyridazin-3-yl)-7-(trifluoromethyl)-2,3-dihydroquinazolin-4(1H)-one;
1-(2-methyl-4-(trifluoromethoxy)phenyl)-3-(3-methylpyridin-4-yl)-6-(trifluoromethyl)-2,3-dihydroquinazolin-4(1H)-one;
3-(6-chloro-4-methylpyridin-3-yl)-1-(4-fluoro-2-methylphenyl)-6-(trifluoromethyl)-2,3-dihydroquinazolin-4(1H)-one;
6-chloro-7-(difluoromethyl)-1-(4-fluoro-2-methylphenyl)-3-(3-methylpyridin-4-yl)-2,3-dihydroquinazolin-4(1H)-one;
1-(4-fluoro-2-methylphenyl)-3-(pyridin-3-yl)-6-(trifluoromethyl)-2,3-dihydroquinazolin-4(1H)-one;
1-(4-fluoro-2-methylphenyl)-3-(4-methylpyridin-3-yl)-6-(trifluoromethyl)-2,3-dihydroquinazolin-4(1H)-one;
1-(4-fluoro-2-methylphenyl)-3-(3-methylpyridazin-4-yl)-6-(trifluoromethyl)-2,3-dihydroquinazolin-4(1H)-one;
7-chloro-6-fluoro-1-(4-fluoro-2-methylphenyl)-3-(6-methoxy-2-methylpyridin-3-yl)-2,3-dihydroquinazolin-4(1H)-one;
1-(4-fluoro-2-methylphenyl)-3-(3-methyl-1H-pyrazol-4-yl)-6-(trifluoromethyl)-2,3-dihydroquinazolin-4(1H)-one;
3-(1-acetylpiperidin-4-yl)-1-(4-fluoro-2-methylphenyl)-6-(trifluoromethyl)-2,3-dihydroquinazolin-4(1H)-one;
1-(4-fluoro-2-methylphenyl)-3-(pyridazin-4-yl)-6-(trifluoromethyl)-2,3-dihydroquinazolin-4(1H)-one;
1-(4-fluoro-2-methylphenyl)-3-(5-methylpyridin-3-yl)-6-(trifluoromethyl)-2,3-dihydroquinazolin-4(1H)-one;
1-(4-fluoro-2-methylphenyl)-3-(2-methylpyridin-3-yl)-6-(trifluoromethyl)-2,3-dihydroquinazolin-4(1H)-one;
1-(4-fluoro-2-methylphenyl)-3-((2R,3S)-2-methyl-6-oxopiperidin-3-yl)-6-(trifluoromethyl)-2,3-dihydroquinazolin-4(1H)-one;
3-(1-(4-fluoro-2-methylphenyl)-4-oxo-6-(trifluoromethyl)-1,4-dihydroquinazolin-3(2H)-yl)piperidine-2,6-dione;
1-(4-Fluoro-2-methylphenyl)-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-3-(6-oxo-1,6-dihydropyridin-3-yl)-7-(trifluoromethyl)-2,3-dihydroquinazolin-4(1H)-one;
1-cyclohexyl-3-(6-oxo-1,6-dihydropyridin-3-yl)-7-(trifluoromethyl)-2,3-dihydroquinazolin-4(1H)-one;
1-(4-fluoro-2,6-dimethylphenyl)-3-(6-oxo-1,6-dihydropyridin-3-yl)-7-(trifluoromethyl)-2,3-dihydroquinazolin-4(1H)-one;
1-(4-fluoro-2-methylphenyl)-2-methyl-3-(6-oxo-1,6-dihydropyridin-3-yl)-7-(trifluoromethyl)-2,3-dihydroquinazolin-4(1H)-one;
1-(2-chloro-4-fluorophenyl)-3-(6-oxo-1,6-dihydropyridin-3-yl)-7-(trifluoromethyl)-2,3-dihydroquinazolin-4(1H)-one;
1-(4-fluoro-2-(2-hydroxyethoxy)phenyl)-3-(6-oxo-1,6-dihydropyridin-3-yl)-7-(trifluoromethyl)-2,3-dihydroquinazolin-4(1H)-one;
1-(2,4-difluorophenyl)-3-(6-oxo-1,6-dihydropyridin-3-yl)-7-(trifluoromethyl)-2,3-dihydroquinazolin-4(1H)-one;
1-(2-ethyl-4-fluorophenyl)-3-(6-oxo-1,6-dihydropyridin-3-yl)-7-(trifluoromethyl)-2,3-dihydroquinazolin-4(1H)-one;
8-chloro-1-(4-fluoro-2-methylphenyl)-3-(6-oxo-1,6-dihydropyridin-3-yl)-7-(trifluoromethyl)-2,3-dihydroquinazolin-4(1H)-one;
1-(4-fluoro-2-methylphenyl)-8-methyl-3-(6-oxo-1,6-dihydropyridin-3-yl)-7-(trifluoromethyl)-2,3-dihydroquinazolin-4(1H)-one;
3-(2-methyl-6-oxo-1,6-dihydropyridin-3-yl)-1-(2-methylpyridin-3-yl)-7-(trifluoromethyl)-2,3-dihydroquinazolin-4(1H)-one;
1-(4-fluoro-2-isopropylphenyl)-3-(2-methyl-6-oxo-1,6-dihydropyridin-3-yl)-7-(trifluoromethyl)-2,3-dihydroquinazolin-4(1H)-one;
6-chloro-1-(4-fluoro-2-methylphenyl)-3-(2-methyl-6-oxo-1,6-dihydropyridin-3-yl)-2,3-dihydroquinazolin-4(1H)-one;
1-(4-fluorophenyl)-3-(2-methyl-6-oxo-1,6-dihydropyridin-3-yl)-2,3-dihydroquinazolin-4(1H)-one;
3-(2-methyl-6-oxo-1,6-dihydropyridin-3-yl)-1-(o-tolyl)-2,3-dihydroquinazolin-4(1H)-one;
1-(4-fluoro-2-methylphenyl)-6-methyl-3-(2-methyl-6-oxo-1,6-dihydropyridin-3-yl)-2,3-dihydroquinazolin-4(1H)-one;
1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-3-(6-methyl-2-oxo-1,2-dihydropyridin-4-yl)-7-(trifluoromethyl)-2,3-dihydroquinazolin-4(1H)-one;
1-(4-fluoro-2-methylphenyl)-3-(5-methyl-2-oxo-1,2-dihydropyridin-4-yl)-7-(trifluoromethyl)-2,3-dihydroquinazolin-4(1H)-one;
1-(4-fluoro-2-methylphenyl)-3-(3-methyl-2-oxo-1,2-dihydropyridin-4-yl)-7-(trifluoromethyl)-2,3-dihydroquinazolin-4(1H)-one;
1-(4-fluoro-2-methylphenyl)-3-(2-methyl-6-oxo-1,6-dihydropyridin-3-yl)-7-(trifluoromethoxy)-2,3-dihydroquinazolin-4(1H)-one;
1-(4-fluoro-2-methylphenyl)-7-methyl-3-(2-methyl-6-oxo-1,6-dihydropyridin-3-yl)-2,3-dihydroquinazolin-4(1H)-one;
1-(4-fluoro-2-methylphenyl)-3-(3-methyl-5-oxo-4,5-dihydropyrazin-2-yl)-7-(trifluoromethyl)-2,3-dihydroquinazolin-4(1H)-one;
6-(1-(4-fluoro-2-methylphenyl)-4-oxo-7-(trifluoromethyl)-1,4-dihydroquinazolin-3(2H)-yl)pyrimidine-2,4(1H,3H)-dione;
1-(2-ethyl-4-fluorophenyl)-3-(2-methyl-6-oxo-1,6-dihydropyridin-3-yl)-6-(trifluoromethyl)-2,3-dihydroquinazolin-4(1H)-one;
6-chloro-1-(4-fluoro-2-methylphenyl)-3-(4-methyl-2-oxo-1,2-dihydropyrimidin-5-yl)-2,3-dihydroquinazolin-4(1H)-one;
3-(2,4-dimethyl-6-oxo-1,6-dihydropyridin-3-yl)-1-(4-fluoro-2-isopropylphenyl)-7-(trifluoromethyl)-2,3-dihydroquinazolin-4(1H)-one;
1-(4-fluoro-2-isopropylphenyl)-3-(6-methoxy-2,4-dimethylpyridin-3-yl)-7-(trifluoromethyl)-2,3-dihydroquinazolin-4(1H)-one;
3-(2-ethyl-6-oxo-1,6-dihydropyridin-3-yl)-1-(4-fluoro-2-methylphenyl)-6-(trifluoromethyl)-2,3-dihydroquinazolin-4(1H)-one;
7-(dimethylamino)-1-(4-fluoro-2-methylphenyl)-3-(2-methyl-6-oxo-1,6-dihydropyridin-3-yl)-2,3-dihydroquinazolin-4(1H)-one;
7-chloro-1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-3-(2-methyl-6-oxo-1,6-dihydropyridin-3-yl)-7-(methylamino)-2,3-dihydroquinazolin-4(1H)-one;
6-chloro-1-(2-ethoxy-4-fluorophenyl)-3-(2-methyl-6-oxo-1,6-dihydropyridin-3-yl)-2,3-dihydroquinazolin-4(1H)-one;
1-(4-fluoro-2-isopropylphenyl)-7-methyl-3-(2-methyl-6-oxo-1,6-dihydropyridin-3-yl)-2,3-dihydroquinazolin-4(1H)-one;
7-(difluoromethyl)-6-fluoro-1-(4-fluoro-2-methylphenyl)-3-(2-methyl-6-oxo-1,6-dihydropyridin-3-yl)-2,3-dihydroquinazolin-4(1H)-one;
6-fluoro-1-(4-fluoro-2-methylphenyl)-7-methyl-3-(2-methyl-6-oxo-1,6-dihydropyridin-3-yl)-2,3-dihydropyrido[2,3-d]pyrimidin-4(1H)-one;
6-chloro-1-(4-fluoro-2-methylphenyl)-7-methyl-3-(2-methyl-6-oxo-1,6-dihydropyridin-3-yl)-2,3-dihydropyrido[2,3-d]pyrimidin-4(1H)-one;
6,7-dichloro-1-(4-fluoro-2-methylphenyl)-3-(2-methyl-6-oxo-1,6-dihydropyridin-3-yl)-2,3-dihydropyrido[2,3-d]pyrimidin-4(1H)-one;
6-chloro-5-fluoro-1-(2-methyl-4-(trifluoromethoxy)phenyl)-3-(2-methyl-6-oxo-1,6-dihydropyridin-3-yl)-2,3-dihydroquinazolin-4(1H)-one;
1-(4-fluoro-2-methylphenyl)-3-(2-methyl-6-oxo-1,6-dihydropyridin-3-yl)-4-oxo-1,2,3,4-tetrahydroquinazoline-6-carbonitrile;
6-chloro-1-(4-fluoro-2-isopropylphenyl)-3-(2-methyl-6-oxo-1,6-dihydropyridin-3-yl)-2,3-dihydroquinazolin-4(1H)-one;
1-(4-fluoro-3-hydroxy-2-methylphenyl)-3-(2-methyl-6-oxo-1,6-dihydropyridin-3-yl)-7-(trifluoromethyl)-2,3-dihydroquinazolin-4(1H)-one;
6-chloro-5-fluoro-1-(4-fluoro-2-methylphenyl)-3-(6-oxo-1,6-dihydropyridin-3-yl)-2,3-dihydroquinazolin-4(1H)-one;
6-chloro-5-fluoro-1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-3-(2-methyl-6-oxo-1,6-dihydropyridin-3-yl)-7-(methylsulfonyl)-2,3-dihydroquinazolin-4(1H)-one;
6-chloro-1-(2-ethyl-4-fluorophenyl)-3-(2-methyl-6-oxo-1,6-dihydropyridin-3-yl)-2,3-dihydroquinazolin-4(1H)-one;
6-chloro-3-(2,4-dimethyl-6-oxo-1,6-dihydropyridin-3-yl)-1-(4-fluoro-2-isopropylphenyl)-2,3-dihydroquinazolin-4(1H)-one;
3-(2-ethyl-6-oxo-1,6-dihydropyridin-3-yl)-1-(4-fluoro-2-isopropylphenyl)-7-(trifluoromethyl)-2,3-dihydroquinazolin-4(1H)-one;
1-(4-fluoro-2-methylphenyl)-3-(5-oxo-4,5-dihydropyrazin-2-yl)-7-(trifluoromethyl)-2,3-dihydroquinazolin-4(1H)-one;
6-fluoro-1-(4-fluoro-2-methylphenyl)-7-methyl-3-(2-methyl-6-oxo-1,6-dihydropyridin-3-yl)-2,3-dihydroquinazolin-4(1H)-one;
3-(2-methyl-6-oxo-1,6-dihydropyridin-3-yl)-1-phenyl-7-(trifluoromethyl)-2,3-dihydroquinazolin-4(1H)-one;
3-(2-methyl-6-oxo-1,6-dihydropyridin-3-yl)-1-(o-tolyl)-7-(trifluoromethyl)-2,3-dihydroquinazolin-4(1H)-one;
5-chloro-1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-5-methyl-3-(2-methyl-6-oxo-1,6-dihydropyridin-3-yl)-2,3-dihydroquinazolin-4(1H)-one;
3-(2-cyclopropyl-6-oxo-1,6-dihydropyridin-3-yl)-1-(4-fluoro-2-methylphenyl)-7-(trifluoromethyl)-2,3-dihydroquinazolin-4(1H)-one;
6-chloro-1-(4-fluoro-2-methoxyphenyl)-3-(2-methyl-6-oxo-1,6-dihydropyridin-3-yl)-2,3-dihydroquinazolin-4(1H)-one;
1-(4-fluoro-2-methylphenyl)-3-(2-methyl-5-oxo-2,5-dihydro-1H-pyrazol-3-yl)-7-(trifluoromethyl)-2,3-dihydroquinazolin-4(1H)-one;
7-fluoro-1-(4-fluoro-2-isopropylphenyl)-3-(2-methyl-6-oxo-1,6-dihydropyridin-3-yl)-2,3-dihydroquinazolin-4(1H)-one;
3-(4,6-dimethyl-2-oxo-1,2-dihydropyrimidin-5-yl)-1-(4-fluoro-2-methylphenyl)-7-(trifluoromethyl)-2,3-dihydroquinazolin-4(1H)-one;
1-(2-(tert-butyl)-4-fluorophenyl)-6-chloro-3-(2-methyl-6-oxo-1,6-dihydropyridin-3-yl)-2,3-dihydroquinazolin-4(1H)-one;
1-(3,4-difluoro-2-methylphenyl)-3-(2-methyl-6-oxo-1,6-dihydropyridin-3-yl)-6-(trifluoromethyl)-2,3-dihydroquinazolin-4(1H)-one;
6-chloro-1-(4-fluoro-2-isopropylphenyl)-5-methyl-3-(2-methyl-6-oxo-1,6-dihydropyridin-3-yl)-2,3-dihydroquinazolin-4(1H)-one;
6-chloro-7-fluoro-1-(4-fluoro-2-isopropylphenyl)-3-(2-methyl-6-oxo-1,6-dihydropyridin-3-yl)-2,3-dihydroquinazolin-4(1H)-one;
1-(4-fluoro-2-methylphenyl)-7-methoxy-3-(2-methyl-6-oxo-1,6-dihydropyridin-3-yl)-2,3-dihydroquinazolin-4(1H)-one;
1-(4-fluoro-2-methylphenyl)-6-hydroxy-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-6-methoxy-3-(2-methyl-6-oxo-1,6-dihydropyridin-3-yl)-7-(trifluoromethyl)-2,3-dihydroquinazolin-4(1H)-one;
6-chloro-1-(4-fluoro-2-methylphenyl)-3-(2-methyl-6-oxo-1,6-dihydropyridin-3-yl)-2,3-dihydropyrido[2,3-d]pyrimidin-4(1H)-one;
1-(4-fluoro-2-isopropylphenyl)-3-(2-methyl-6-oxo-1,6-dihydropyridin-3-yl)-7-(trifluoromethoxy)-2,3-dihydroquinazolin-4(1H)-one;
6-chloro-7-fluoro-1-(4-fluoro-2-methylphenyl)-3-(2-methyl-6-oxo-1,6-dihydropyridin-3-yl)-2,3-dihydroquinazolin-4(1H)-one;
6-chloro-1-(4-fluoro-2-methylphenyl)-3-(2-methyl-6-oxo-1,6-dihydropyridin-3-yl)-4-oxo-1,2,3,4-tetrahydroquinazoline-7-carbonitrile;
6-chloro-3-(2-ethyl-6-oxo-1,6-dihydropyridin-3-yl)-1-(4-fluoro-2-methylphenyl)-2,3-dihydroquinazolin-4(1H)-one;
7-fluoro-1-(4-fluoro-2-methylphenyl)-3-(2-methyl-6-oxo-1,6-dihydropyridin-3-yl)-6-(trifluoromethyl)-2,3-dihydroquinazolin-4(1H)-one;
5-fluoro-1-(4-fluoro-2-methylphenyl)-3-(2-methyl-6-oxo-1,6-dihydropyridin-3-yl)-6-(trifluoromethyl)-2,3-dihydroquinazolin-4(1H)-one;
7-chloro-6-fluoro-1-(4-fluoro-2-methylphenyl)-3-(2-methyl-6-oxo-1,6-dihydropyridin-3-yl)-2,3-dihydroquinazolin-4(1H)-one;
6-fluoro-1-(4-fluoro-2-methylphenyl)-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-3-(2-methyl-6-oxo-1,6-dihydropyridin-3-yl)-4-oxo-6-(trifluoromethyl)-1,2,3,4-tetrahydroquinazoline-7-carbonitrile;
1-(4-fluoro-2-methylphenyl)-3-(2-methyl-6-oxo-1,6-dihydropyridin-3-yl)-4-oxo-7-(trifluoromethoxy)-1,2,3,4-tetrahydroquinazoline-6-carbonitrile;
1-(4,5-difluoro-2-methylphenyl)-3-(2-methyl-6-oxo-1,6-dihydropyridin-3-yl)-6-(trifluoromethyl)-2,3-dihydroquinazolin-4(1H)-one;
1-(2-(tert-butyl)-4-fluorophenyl)-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-3-(6-methyl-2-oxo-1,2-dihydropyrimidin-5-yl)-7-(trifluoromethyl)-2,3-dihydroquinazolin-4(1H)-one;
1-(2-ethyl-4-fluorophenyl)-6-fluoro-3-(2-methyl-6-oxo-1,6-dihydropyridin-3-yl)-2,3-dihydroquinazolin-4(1H)-one;
4-(6-chloro-3-(2-methyl-6-oxo-1,6-dihydropyridin-3-yl)-4-oxo-3,4-dihydroquinazolin-1(2H)-yl)-3-methylbenzonitrile;
1-(5-fluoro-2-methylphenyl)-3-(2-methyl-6-oxo-1,6-dihydropyridin-3-yl)-6-(trifluoromethyl)-2,3-dihydroquinazolin-4(1H)-one;
1-(2-methyl-4-(trifluoromethoxy)phenyl)-3-(6-oxo-1,6-dihydropyridin-3-yl)-6-(trifluoromethyl)-2,3-dihydropyrido[2,3-d]pyrimidin-4(1H)-one;
1-(3-fluoro-2-methylphenyl)-3-(2-methyl-6-oxo-1,6-dihydropyridin-3-yl)-6-(trifluoromethyl)-2,3-dihydroquinazolin-4(1H)-one;
1-(2,4-difluoro-6-methylphenyl)-3-(2-methyl-6-oxo-1,6-dihydropyridin-3-yl)-6-(trifluoromethyl)-2,3-dihydroquinazolin-4(1H)-one;
7-(difluoromethyl)-1-(4-fluoro-2-isopropylphenyl)-3-(2-methyl-6-oxo-1,6-dihydropyridin-3-yl)-2,3-dihydropyrido[2,3-d]pyrimidin-4(1H)-one;
3-methyl-4-(3-(2-methyl-6-oxo-1,6-dihydropyridin-3-yl)-4-oxo-6-(trifluoromethoxy)-3,4-dihydroquinazolin-1(2H)-yl)benzonitrile;
8-chloro-1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-8-methyl-3-(2-methyl-6-oxo-1,6-dihydropyridin-3-yl)-2,3-dihydroquinazolin-4(1H)-one;
1-(4-methoxy-2-methylphenyl)-3-(2-methyl-6-oxo-1,6-dihydropyridin-3-yl)-6-(trifluoromethyl)-2,3-dihydroquinazolin-4(1H)-one;
1-(4-hydroxy-2-methylphenyl)-3-(2-methyl-6-oxo-1,6-dihydropyridin-3-yl)-6-(trifluoromethyl)-2,3-dihydroquinazolin-4(1H)-one;
3-(6-chloro-3-(2-methyl-6-oxo-1,6-dihydropyridin-3-yl)-4-oxo-3,4-dihydroquinazolin-1(2H)-yl)-2-methylbenzonitrile;
6-fluoro-1-(4-fluoro-2-methylphenyl)-3-(2-methyl-6-oxo-1,6-dihydropyridin-3-yl)-7-(trifluoromethoxy)-2,3-dihydroquinazolin-4(1H)-one;
5-(difluoromethyl)-1-(4-fluoro-2-methylphenyl)-3-(2-methyl-6-oxo-1,6-dihydropyridin-3-yl)-2,3-dihydroquinazolin-4(1H)-one;
3-(5-fluoro-2-methyl-6-oxo-1,6-dihydropyridin-3-yl)-1-(4-fluoro-2-methylphenyl)-6-(trifluoromethyl)-2,3-dihydroquinazolin-4(1H)-one;
6,7-difluoro-1-(4-fluoro-2-methoxyphenyl)-3-(2-methyl-6-oxo-1,6-dihydropyridin-3-yl)-2,3-dihydroquinazolin-4(1H)-one;
6-chloro-1-(2-hydroxy-4-methylphenyl)-3-(2-methyl-6-oxo-1,6-dihydropyridin-3-yl)-2,3-dihydroquinazolin-4(1H)-one;
6-chloro-1-(2-methoxy-4-methylphenyl)-3-(2-methyl-6-oxo-1,6-dihydropyridin-3-yl)-2,3-dihydroquinazolin-4(1H)-one;
1-(2-fluoro-6-methylphenyl)-3-(2-methyl-6-oxo-1,6-dihydropyridin-3-yl)-6-(trifluoromethyl)-2,3-dihydroquinazolin-4(1H)-one;
6-chloro-5-fluoro-1-(4-fluoro-2-methylphenyl)-3-(2-oxo-1,2-dihydropyridin-4-yl)-2,3-dihydroquinazolin-4(1H)-one;
7-(difluoromethyl)-1-(4-fluoro-2-isopropylphenyl)-3-(2-methyl-6-oxo-1,6-dihydropyridin-3-yl)-2,3-dihydroquinazolin-4(1H)-one;
7-(difluoromethyl)-1-(4-fluoro-2-methylphenyl)-3-(2-methyl-6-oxo-1,6-dihydropyridin-3-yl)-2,3-dihydroquinazolin-4(1H)-one;
6-chloro-3-(2-ethyl-6-oxo-1,6-dihydropyridin-3-yl)-1-(4-fluoro-2-isopropylphenyl)-2,3-dihydroquinazolin-4(1H)-one;
6-fluoro-2-methyl-3-(3-(2-methyl-6-oxo-1,6-dihydropyridin-3-yl)-4-oxo-6-(trifluoromethyl)-3,4-dihydroquinazolin-1(2H)-yl)benzonitrile;
6-fluoro-1-(4-fluoro-2-isopropylphenyl)-3-(2-methyl-6-oxo-1,6-dihydropyridin-3-yl)-2,3-dihydropyrido[2,3-d]pyrimidin-4(1H)-one;
1-(2-bromo-4-fluorophenyl)-3-(2-methyl-6-oxo-1,6-dihydropyridin-3-yl)-6-(trifluoromethyl)-2,3-dihydroquinazolin-4(1H)-one;
6-fluoro-1-(4-fluoro-2-methylphenyl)-3-(2-methyl-6-oxo-1,6-dihydropyridin-3-yl)-2,3-dihydroquinazolin-4(1H)-one;
1-(4-Fluorophenyl)-3-(6-oxo-1,6-dihydropyridin-3-yl)-7-(trifluoromethyl)-2,3-dihydroquinazolin-4(1H)-one;
1-(4-fluoro-2-methylphenyl)-3-(2-oxo-1,2-dihydropyrimidin-5-yl)-7-(trifluoromethyl)-2,3-dihydroquinazolin-4(1H)-one;
1-(4-bromo-2-methylphenyl)-3-(6-oxo-1,6-dihydropyridin-3-yl)-7-(trifluoromethyl)-2,3-dihydroquinazolin-4(1H)-one;
1-(4-fluorophenyl)-3-(2-methyl-6-oxo-1,6-dihydropyridin-3-yl)-7-(trifluoromethyl)-2,3-dihydroquinazolin-4(1H)-one;
3-methyl-4-(3-(2-methyl-6-oxo-1,6-dihydropyridin-3-yl)-4-oxo-7-(trifluoromethyl)-3,4-dihydroquinazolin-1(2H)-yl)benzonitrile;
1-(4-fluoro-2-methylphenyl)-3-(2-methyl-6-oxo-1,6-dihydropyridin-3-yl)-7-(trifluoromethyl)-2,3-dihydropyrido[2,3-d]pyrimidin-4(1H)-one;
3-(2-ethyl-6-oxo-1,6-dihydropyridin-3-yl)-1-(4-fluoro-2-methylphenyl)-7-(trifluoromethyl)-2,3-dihydroquinazolin-4(1H)-one;
3-(2-chloro-6-oxo-1,6-dihydropyridin-3-yl)-1-(4-fluoro-2-methylphenyl)-7-(trifluoromethyl)-2,3-dihydroquinazolin-4(1H)-one;
7-bromo-1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-3-(2-methyl-6-oxo-1,6-dihydropyridin-3-yl)-4-oxo-1,2,3,4-tetrahydroquinazoline-7-carbonitrile;
3-(1-(4-fluoro-2-methylphenyl)-4-oxo-7-(trifluoromethyl)-1,4-dihydroquinazolin-3(2H)-yl)-6-oxo-1,6-dihydropyridine-2-carbonitrile;
1-(2,4-difluorophenyl)-3-(2-methyl-6-oxo-1,6-dihydropyridin-3-yl)-7-(trifluoromethyl)-2,3-dihydroquinazolin-4(1H)-one;
1-(4-ethoxyphenyl)-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-3-(2-oxo-1,2-dihydropyridin-4-yl)-7-(trifluoromethyl)-2,3-dihydroquinazolin-4(1H)-one;
6,7-difluoro-1-(4-fluoro-2-methylphenyl)-3-(2-methyl-6-oxo-1,6-dihydropyridin-3-yl)-2,3-dihydroquinazolin-4(1H)-one;
6,7-difluoro-1-(4-fluoro-2-isopropylphenyl)-3-(2-methyl-6-oxo-1,6-dihydropyridin-3-yl)-2,3-dihydroquinazolin-4(1H)-one;
3-(2-methyl-6-oxo-1,6-dihydropyridin-3-yl)-1-((1S,2R)-2-methylcyclohexyl)-7-(trifluoromethyl)-2,3-dihydroquinazolin-4(1H)-one;
1-(4-fluoro-2-isopropylphenyl)-3-(2-methyl-6-oxo-1,6-dihydropyridin-3-yl)-4-oxo-1,2,3,4-tetrahydroquinazoline-7-carbonitrile;
3-(2-methyl-6-oxo-1,6-dihydropyridin-3-yl)-1-((1R,2S)-2-methylcyclohexyl)-7-(trifluoromethyl)-2,3-dihydroquinazolin-4(1H)-one;
N-(3-(1-(4-fluoro-2-methylphenyl)-4-oxo-6-(trifluoromethyl)-1,4-dihydroquinazolin-3(2H)-yl)-6-oxo-1,6-dihydropyridin-2-yl)acetamide;
3-(2-bromo-6-oxo-1,6-dihydropyridin-3-yl)-1-(4-fluoro-2-methylphenyl)-6-(trifluoromethyl)-2,3-dihydroquinazolin-4(1H)-one;
6-fluoro-1-(4-fluoro-2-methylphenyl)-7-methoxy-3-(2-methyl-6-oxo-1,6-dihydropyridin-3-yl)-2,3-dihydropyrido[2,3-d]pyrimidin-4(1H)-one;
1-(4-fluoro-2-methylphenyl)-3-(2-methyl-6-oxo-1,6-dihydropyridin-3-yl)-4-oxo-7-(trifluoromethyl)-1,2,3,4-tetrahydroquinazoline-6-carbonitrile;
6-chloro-7-(difluoromethoxy)-1-(4-fluoro-2-methylphenyl)-3-(2-methyl-6-oxo-1,6-dihydropyridin-3-yl)-2,3-dihydroquinazolin-4(1H)-one;
6-chloro-7-(difluoromethyl)-1-(4-fluoro-2-methylphenyl)-3-(2-methyl-6-oxo-1,6-dihydropyridin-3-yl)-2,3-dihydroquinazolin-4(1H)-one;
3-(4-amino-2-oxo-1,2-dihydropyrimidin-5-yl)-1-(4-fluoro-2-methylphenyl)-6-(trifluoromethyl)-2,3-dihydroquinazolin-4(1H)-one;
6-chloro-1-(2-methyl-4-(trifluoromethoxy)phenyl)-3-(2-methyl-6-oxo-1,6-dihydropyridin-3-yl)-4-oxo-1,2,3,4-tetrahydropyrido[2,3-d]pyrimidine-7-carbonitrile;
6-chloro-1-(4-fluoro-2-methylphenyl)-3-(2-methyl-6-oxo-1,6-dihydropyridin-3-yl)-4-oxo-1,2,3,4-tetrahydropyrido[2,3-d]pyrimidine-7-carbonitrile;
6-chloro-1-(2-methyl-4-(trifluoromethoxy)phenyl)-3-(2-methyl-6-oxo-1,6-dihydropyridin-3-yl)-4-oxo-1,2,3,4-tetrahydroquinazoline-7-carbonitrile;
6-chloro-1-(4-fluoro-2-methylphenyl)-3-(6-methyl-2-oxo-1,2-dihydropyrimidin-5-yl)-4-oxo-1,2,3,4-tetrahydroquinazoline-7-carbonitrile;
1-(2-methyl-3-(trifluoromethyl)phenyl)-3-(2-methyl-6-oxo-1,6-dihydropyridin-3-yl)-6-(trifluoromethyl)-2,3-dihydropyrido[2,3-d]pyrimidin-4(1H)-one;
1-(3-chloro-2-methylphenyl)-3-(2-methyl-6-oxo-1,6-dihydropyridin-3-yl)-6-(trifluoromethyl)-2,3-dihydropyrido[2,3-d]pyrimidin-4(1H)-one;
1-(2-ethyl-4-fluorophenyl)-3-(2-methyl-6-oxo-1,6-dihydropyridin-3-yl)-6-(trifluoromethyl)-2,3-dihydropyrido[2,3-d]pyrimidin-4(1H)-one;
1-(3,4-difluoro-2-methylphenyl)-3-(2-methyl-6-oxo-1,6-dihydropyridin-3-yl)-6-(trifluoromethyl)-2,3-dihydropyrido[2,3-d]pyrimidin-4(1H)-one;
1-(4-fluoro-2-isopropylphenyl)-3-(2-methyl-6-oxo-1,6-dihydropyridin-3-yl)-6-(trifluoromethyl)-2,3-dihydroquinazolin-4(1H)-one;
1-(4-fluoro-2-methylphenyl)-3-(4-methyl-6-oxo-1,6-dihydropyridin-3-yl)-6-(trifluoromethyl)-2,3-dihydroquinazolin-4(1H)-one;
6-fluoro-1-(4-fluoro-2-isopropylphenyl)-3-(2-methyl-6-oxo-1,6-dihydropyridin-3-yl)-2,3-dihydroquinazolin-4(1H)-one;
1-(2-cyclopropyl-4-fluorophenyl)-3-(2-methyl-6-oxo-1,6-dihydropyridin-3-yl)-2,3-dihydroquinazolin-4(1H)-one;
1-(2-cyclopropyl-4-fluorophenyl)-3-(2-methyl-6-oxo-1,6-dihydropyridin-3-yl)-7-(trifluoromethyl)-2,3-dihydroquinazolin-4(1H)-one;
5-fluoro-1-(4-fluoro-2-methylphenyl)-3-(2-methyl-6-oxo-1,6-dihydropyridin-3-yl)-2,3-dihydroquinazolin-4(1H)-one;
1-(2-ethyl-4-fluorophenyl)-3-(2-methyl-6-oxo-1,6-dihydropyridin-3-yl)-7-(trifluoromethyl)-2,3-dihydroquinazolin-4(1H)-one;
7-(difluoromethoxy)-1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-3-(6-oxo-1,6-dihydropyridin-3-yl)-7-(trifluoromethyl)quinazoline-2,4(1H,3H)-dione;
7-cyclopropyl-1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-3-(2-methyl-6-oxo-1,6-dihydropyridin-3-yl)-6-(trifluoromethoxy)-2,3-dihydroquinazolin-4(1H)-one;
1-(4-(difluoromethoxy)-2-methylphenyl)-3-(2-methyl-6-oxo-1,6-dihydropyridin-3-yl)-6-(trifluoromethyl)-2,3-dihydroquinazolin-4(1H)-one;
3-(2-ethyl-6-oxo-1,6-dihydropyridin-3-yl)-1-(4-fluoro-2-methylphenyl)-7-(trifluoromethoxy)-2,3-dihydroquinazolin-4(1H)-one;
6-chloro-1-(2-ethyl-4-fluorophenyl)-7-fluoro-3-(2-methyl-6-oxo-1,6-dihydropyridin-3-yl)-2,3-dihydroquinazolin-4(1H)-one;
6-chloro-1-(2-ethyl-4-fluorophenyl)-3-(2-methyl-6-oxo-1,6-dihydropyridin-3-yl)-4-oxo-1,2,3,4-tetrahydroquinazoline-7-carbonitrile;
6-chloro-5,7-difluoro-1-(4-fluoro-2-methylphenyl)-3-(2-methyl-6-oxo-1,6-dihydropyridin-3-yl)-2,3-dihydroquinazolin-4(1H)-one;
6-chloro-5-fluoro-1-(4-fluoro-2-methylphenyl)-3-(2-methyl-6-oxo-1,6-dihydropyridin-3-yl)-4-oxo-1,2,3,4-tetrahydroquinazoline-7-carbonitrile;
1-(2-methyl-4-(trifluoromethyl)phenyl)-3-(2-methyl-6-oxo-1,6-dihydropyridin-3-yl)-6-(trifluoromethyl)-2,3-dihydropyrido[2,3-d]pyrimidin-4(1H)-one;
1-(4-chloro-2-methylphenyl)-3-(2-methyl-6-oxo-1,6-dihydropyridin-3-yl)-6-(trifluoromethyl)-2,3-dihydropyrido[2,3-d]pyrimidin-4(1H)-one;
7-(1,1-difluoro-2-hydroxyethyl)-1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-3-(2-isopropyl-6-oxo-1,6-dihydropyridin-3-yl)-7-(trifluoromethyl)-2,3-dihydroquinazolin-4(1H)-one;
1-(4-fluoro-2-methylphenyl)-3-(2-((2-hydroxyethyl)amino)-6-oxo-1,6-dihydropyridin-3-yl)-7-(trifluoromethyl)-2,3-dihydroquinazolin-4(1H)-one;
6-chloro-1-(2-(dimethylamino)-4-fluorophenyl)-3-(2-methyl-6-oxo-1,6-dihydropyridin-3-yl)-2,3-dihydroquinazolin-4(1H)-one;
6-chloro-1-(4-fluoro-2-(methylamino)phenyl)-3-(2-methyl-6-oxo-1,6-dihydropyridin-3-yl)-2,3-dihydroquinazolin-4(1H)-one;
1-(4-(1,1-difluoro-2-hydroxyethyl)-2-methylphenyl)-3-(2-methyl-6-oxo-1,6-dihydropyridin-3-yl)-7-(trifluoromethyl)-2,3-dihydroquinazolin-4(1H)-one;
1-(2,4-dimethylphenyl)-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-3-(2-oxo-1,2-dihydropyridin-3-yl)-7-(trifluoromethyl)-2,3-dihydroquinazolin-4(1H)-one;
1-(4-fluoro-2-methylphenyl)-3-(6-methyl-2-oxo-1,2-dihydropyrimidin-5-yl)-7-(trifluoromethoxy)-2,3-dihydroquinazolin-4(1H)-one;
1-(4-fluoro-2-methylphenyl)-7-methyl-3-(2-methyl-6-oxo-1,6-dihydropyridin-3-yl)-2,3-dihydropyrido[2,3-d]pyrimidin-4(1H)-one;
6-chloro-1-(2-ethyl-4-fluorophenyl)-3-(2-methyl-6-oxo-1,6-dihydropyridin-3-yl)-4-oxo-1,2,3,4-tetrahydropyrido[2,3-d]pyrimidine-7-carbonitrile;
1-((1S,3S)-3-fluorocyclopentyl)-3-(2-methyl-6-oxo-1,6-dihydropyridin-3-yl)-7-(trifluoromethyl)-2,3-dihydroquinazolin-4(1H)-one;
1-((1R,3R)-3-fluorocyclopentyl)-3-(2-methyl-6-oxo-1,6-dihydropyridin-3-yl)-7-(trifluoromethyl)-2,3-dihydroquinazolin-4(1H)-one;
3-(1,2-dimethyl-6-oxo-1,6-dihydropyridin-3-yl)-1-(4-fluoro-2-methylphenyl)-6-(trifluoromethyl)-2,3-dihydroquinazolin-4(1H)-one;
3-(2-chloro-6-oxo-1,6-dihydropyridin-3-yl)-1-(4-fluoro-2-methylphenyl)-6-(trifluoromethyl)-2,3-dihydroquinazolin-4(1H)-one;
3-(2-bromo-4-methyl-6-oxo-1,6-dihydropyridin-3-yl)-1-(4-fluoro-2-methylphenyl)-7-(trifluoromethyl)-2,3-dihydroquinazolin-4(1H)-one;
1-(4-fluoro-2-isopropylphenyl)-3-(2-methyl-6-oxo-1,6-dihydropyridin-3-yl)-7-(trifluoromethyl)-2,3-dihydropyrido[2,3-d]pyrimidin-4(1H)-one;
6-chloro-1-(4-fluoro-2-isopropylphenyl)-3-(2-methyl-6-oxo-1,6-dihydropyridin-3-yl)-2,3-dihydropyrido[2,3-d]pyrimidin-4(1H)-one;
5-(1-(4-fluoro-2-methylphenyl)-4-thioxo-7-(trifluoromethyl)-1,4-dihydroquinazolin-3(2H)-yl)-6-methylpyridin-2(1H)-one;
1-(4-Fluoro-2-methylphenyl)-3-(6-oxo-1,6-dihydropyridin-3-yl)-6-(trifluoromethyl)-2,3-dihydroquinazolin-4(1H)-one;
1-(4-fluoro-2-methoxyphenyl)-3-(6-oxo-1,6-dihydropyridin-3-yl)-7-(trifluoromethyl)-2,3-dihydroquinazolin-4(1H)-one;
1-(4-fluoro-2-methylphenyl)-3-(6-oxo-1,6-dihydropyridin-3-yl)-5-(trifluoromethyl)-2,3-dihydroquinazolin-4(1H)-one;
1-(4-fluoro-2-methylphenyl)-3-(5-methyl-6-oxo-1,6-dihydropyridin-3-yl)-7-(trifluoromethyl)-2,3-dihydroquinazolin-4(1H)-one;
1-(4-fluoro-2-methylphenyl)-3-(6-oxo-1,6-dihydropyridazin-3-yl)-7-(trifluoromethyl)-2,3-dihydroquinazolin-4(1H)-one;
1-(4-fluorobenzyl)-3-(6-oxo-1,6-dihydropyridin-3-yl)-7-(trifluoromethyl)-2,3-dihydroquinazolin-4(1H)-one;
1-(4-fluoro-2-methylphenyl)-3-(4-methyl-6-oxo-1,6-dihydropyridin-3-yl)-7-(trifluoromethyl)-2,3-dihydroquinazolin-4(1H)-one;
1-(4-fluoro-2-methylphenyl)-4-oxo-3-(6-oxo-1,6-dihydropyridin-3-yl)-1,2,3,4-tetrahydroquinazoline-7-carbonitrile;
1-(4-fluoro-2-methoxyphenyl)-3-(6-oxo-1,6-dihydropyridin-3-yl)-5-(trifluoromethyl)-2,3-dihydroquinazolin-4(1H)-one;
3-(2-methyl-6-oxo-1,6-dihydropyridin-3-yl)-1-(4-methylthiazol-5-yl)-6-(trifluoromethyl)-2,3-dihydroquinazolin-4(1H)-one;
1-(2,4-dimethoxyphenyl)-3-(2-methyl-6-oxo-1,6-dihydropyridin-3-yl)-6-(trifluoromethyl)-2,3-dihydroquinazolin-4(1H)-one;
1-(4-fluoro-2-methoxyphenyl)-3-(2-methyl-6-oxo-1,6-dihydropyridin-3-yl)-6-(trifluoromethyl)-2,3-dihydroquinazolin-4(1H)-one;
7-chloro-6-fluoro-1-(4-fluoro-2-methylphenyl)-3-(2-methyl-6-oxo-1,6-dihydropyridin-3-yl)-2,3-dihydropyrido[2,3-d]pyrimidin-4(1H)-one;
3-(2-methyl-6-oxo-1,6-dihydropyridin-3-yl)-1-(3-methylthiophen-2-yl)-6-(trifluoromethyl)-2,3-dihydroquinazolin-4(1H)-one;
1-(4-fluoro-2-methoxyphenyl)-4-oxo-3-(6-oxo-1,6-dihydropyridin-3-yl)-1,2,3,4-tetrahydroquinazoline-7-carbonitrile;
1-(4-fluoro-2-methylphenyl)-3-(2-methyl-6-oxo-1,6-dihydropyridin-3-yl)-7-(2,2,2-trifluoroethoxy)-2,3-dihydroquinazolin-4(1H)-one;
1-(2-methyl-4-(trifluoromethoxy)phenyl)-3-(6-oxo-1,6-dihydropyridin-3-yl)-6-(trifluoromethyl)-2,3-dihydroquinazolin-4(1H)-one;
6-chloro-3-(4-chloro-2-methyl-6-oxo-1,6-dihydropyridin-3-yl)-1-(4-fluoro-2-methylphenyl)-2,3-dihydroquinazolin-4(1H)-one;
7-chloro-1-(4-fluoro-2-methylphenyl)-3-(2-methyl-6-oxo-1,6-dihydropyridin-3-yl)-4-oxo-1,2,3,4-tetrahydroquinazoline-6-carbonitrile;
6-chloro-1-(4-fluoro-2-methylphenyl)-5-methyl-3-(2-methyl-6-oxo-1,6-dihydropyridin-3-yl)-2,3-dihydroquinazolin-4(1H)-one;
1-(3,5-difluoro-2-methylphenyl)-3-(2-methyl-6-oxo-1,6-dihydropyridin-3-yl)-6-(trifluoromethyl)-2,3-dihydroquinazolin-4(1H)-one;
1-(4-fluoro-2-isopropylphenyl)-5-methoxy-3-(2-methyl-6-oxo-1,6-dihydropyridin-3-yl)-2,3-dihydroquinazolin-4(1H)-one;
1-(bicyclo[1.1.1]pentan-1-yl)-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-3-(3-methyl-2-oxo-1,2-dihydropyridin-4-yl)-6-(trifluoromethyl)-2,3-dihydroquinazolin-4(1H)-one;
5-(1-(4-fluoro-2-methylphenyl)-6-(trifluoromethyl)-1,4-dihydroquinazolin-3(2H)-yl)-6-methylpyridin-2(1H)-one;
3-(2-Methyl-6-oxo-1,6-dihydropyridin-3-yl)-1-(2-(2,2,2-trifluoroethyl)phenyl)-6-(trifluoromethyl)-2,3-dihydroquinazolin-4(1H)-one;
7-(difluoromethoxy)-1-(4-fluoro-2-methylphenyl)-3-(2-methyl-6-oxo-1,6-dihydropyridin-3-yl)-2,3-dihydropyrido[2,3-d]pyrimidin-4(1H)-one;
6-fluoro-1-(4-fluoro-2-methylphenyl)-3-(2-methyl-6-oxo-1,6-dihydropyridin-3-yl)-4-oxo-1,2,3,4-tetrahydroquinazoline-7-carbonitrile;
6-chloro-1-(2-ethyl-4-fluorophenyl)-3-(3-methylpyridin-4-yl)-4-oxo-1,2,3,4-tetrahydroquinazoline-7-carbonitrile;
4-(6-Chloro-7-(difluoromethyl)-1-(4-fluoro-2-methylphenyl)-4-oxo-1,4-dihydroquinazolin-3(2H)-yl)-3-methylpyridine 1-oxide;
4-(6-chloro-1-(2-ethyl-4-fluorophenyl)-7-fluoro-4-oxo-1,4-dihydroquinazolin-3(2H)-yl)-3-methylpyridine 1-oxide;
4-(6-chloro-7-fluoro-1-(2-methyl-4-(trifluoromethoxy)phenyl)-4-oxo-1,4-dihydroquinazolin-3(2H)-yl)-3-methylpyridine 1-oxide;
4-(6-chloro-7-cyano-1-(2-ethyl-4-fluorophenyl)-4-oxo-1,4-dihydroquinazolin-3(2H)-yl)-3-methylpyridine 1-oxide;
2-carbamoyl-5-(6-chloro-5-fluoro-1-(4-fluoro-2-methylphenyl)-4-oxo-1,4-dihydroquinazolin-3(2H)-yl)pyridine 1-oxide;
3-(1-(4-fluoro-2-methylphenyl)-4-oxo-6-(trifluoromethyl)-1,4-dihydroquinazolin-3(2H)-yl)pyridine 1-oxide;
3-(1-(4-fluoro-2-methylphenyl)-4-oxo-6-(trifluoromethyl)-1,4-dihydroquinazolin-3(2H)-yl)-4-methylpyridine 1-oxide;
5-(1-(4-fluoro-2-methylphenyl)-4-oxo-6-(trifluoromethyl)-1,4-dihydroquinazolin-3(2H)-yl)pyridazine 1-oxide;
4-(1-(4-fluoro-2-methylphenyl)-4-oxo-6-(trifluoromethyl)-1,4-dihydroquinazolin-3(2H)-yl)pyridazine 1-oxide;
3-(1-(4-fluoro-2-methylphenyl)-4-oxo-6-(trifluoromethyl)-1,4-dihydroquinazolin-3(2H)-yl)-2-methylpyridine 1-oxide;
4-(1-(4-fluoro-2-methylphenyl)-4-oxo-6-(trifluoromethyl)-1,4-dihydroquinazolin-3(2H)-yl)-3-methylpyridine 1-oxide;
3-(1-(4-fluoro-2-methylphenyl)-4-oxo-6-(trifluoromethyl)-1,4-dihydroquinazolin-3(2H)-yl)-5-methylpyridine 1-oxide;
3-methyl-4-(1-(2-methyl-4-(trifluoromethoxy)phenyl)-4-oxo-6-(trifluoromethyl)-1,4-dihydroquinazolin-3(2H)-yl)pyridine 1-oxide;
3-(2-Amino-6-oxo-1,6-dihydropyridin-3-yl)-1-(4-fluoro-2-methylphenyl)-6-(trifluoromethyl)-2,3-dihydroquinazolin-4(1H)-one;
3-(6-Amino-2-methylpyridin-3-yl)-1-(4-fluoro-2-methylphenyl)-6-(trifluoromethyl)-2,3-dihydroquinazolin-4(1H)-one;
2-((5-(6-Chloro-1-(4-fluoro-2-methylphenyl)-4-oxo-1,4-dihydroquinazolin-3(2H)-yl)-6-methylpyridin-2-yl)oxy)acetic acid;
1-(4-Fluoro-2-methylphenyl)-3-(2-methoxy-6-oxo-1,6-dihydropyridin-3-yl)-7-(trifluoromethyl)-2,3-dihydroquinazolin-4(1H)-one;
3-(6-Aminopyridin-3-yl)-1-(4-fluoro-2-methylphenyl)-7-(trifluoromethyl)-2,3-dihydroquinazolin-4(1H)-one;
4-(1-(4-Fluoro-2-methylphenyl)-4-oxo-7-(trifluoromethyl)-1,4-dihydroquinazolin-3(2H)-yl)benzoic acid;
3-(1-(4-fluoro-2-methylphenyl)-4-oxo-7-(trifluoromethyl)-1,4-dihydroquinazolin-3(2H)-yl)benzoic acid;
1-(4-Fluoro-2-methylphenyl)-3-(2-hydroxy-6-oxo-1,6-dihydropyridin-3-yl)-7-(trifluoromethyl)-2,3-dihydroquinazolin-4(1H)-one;
1-(4-Fluoro-2-methylphenyl)-3-(2-methyl-6-oxo-1,6-dihydropyridin-3-yl)-7-(oxetan-3-yl)-2,3-dihydroquinazolin-4(1H)-one;
1-(4-Fluoro-2-methylphenyl)-3-(4-hydroxy-2-methylphenyl)-6-(trifluoromethyl)-2,3-dihydroquinazolin-4(1H)-one;
1-(4-Fluoro-2-methylphenyl)-3-(4-hydroxy-2-methylphenyl)-7-(trifluoromethyl)-2,3-dihydroquinazolin-4(1H)-one;
1-(4-Fluorophenyl)-3-(6-oxo-1,6-dihydropyridin-3-yl)-7-(trifluoromethyl)-2,3-dihydroquinazolin-4(1H)-one;
1-(4-fluoro-2-methylphenyl)-3-(2-methyl-6-oxo-1,4,5,6-tetrahydropyridin-3-yl)-7-(trifluoromethyl)-2,3-dihydroquinazolin-4(1H)-one;
1-(4-fluoro-2-methylphenyl)-3-(2-oxohexahydropyrimidin-5-yl)-7-(trifluoromethyl)-2,3-dihydroquinazolin-4(1H)-one;
1-(4-fluoro-2-methylphenyl)-3-(6-methyl-2-oxo-1,2,3,4-tetrahydropyrimidin-5-yl)-7-(trifluoromethyl)-2,3-dihydroquinazolin-4(1H)-one;
3-(1-(4-Fluoro-2-methylphenyl)-4-oxo-7-(trifluoromethyl)-1,4-dihydroquinazolin-3(2H)-yl)benzamide;
3-(6-chloro-1-(4-fluoro-2-methylphenyl)-4-oxo-1,4-dihydroquinazolin-3(2H)-yl)furan-2-carboxamide;
4-Methyl-3-(3-(2-methyl-6-oxo-1,6-dihydropyridin-3-yl)-4-oxo-6-(trifluoromethyl)-3,4-dihydroquinazolin-1(2H)-yl)benzamide;
4-(6-Chloro-1-(4-fluoro-2-methylphenyl)-4-oxo-1,4-dihydroquinazolin-3(2H)-yl)furan-2-carboxamide;
3-(1-(4-Fluoro-2-methylphenyl)-4-oxo-7-(trifluoromethyl)-1,4-dihydroquinazolin-3(2H)-yl)benzenesulfonamide;
3-(6-Amino-4-methylpyridin-3-yl)-1-(4-fluoro-2-methylphenyl)-6-(trifluoromethyl)-2,3-dihydroquinazolin-4(1H)-one;
3-(2-Methyl-6-oxo-1,6-dihydropyridin-3-yl)-1-((1S,2S)-2-methylcyclohexyl)-7-(trifluoromethyl)-2,3-dihydroquinazolin-4(1H)-one;
3-(2-Methyl-6-oxo-1,6-dihydropyridin-3-yl)-1-((1R,2R)-2-methylcyclohexyl)-7-(trifluoromethyl)-2,3-dihydroquinazolin-4(1H)-one;
3-(2-Methyl-6-oxo-1,6-dihydropyridin-3-yl)-1-((3S,4S)-3-methyltetrahydro-2H-pyran-4-yl)-7-(trifluoromethyl)-2,3-dihydroquinazolin-4(1H)-one;
3-(2-Methyl-6-oxo-1,6-dihydropyridin-3-yl)-1-((3R,4R)-3-methyltetrahydro-2H-pyran-4-yl)-7-(trifluoromethyl)-2,3-dihydroquinazolin-4(1H)-one;
3-(2-Methyl-6-oxo-1,6-dihydropyridin-3-yl)-1-((3R,4S)-3-methyltetrahydro-2H-pyran-4-yl)-7-(trifluoromethyl)-2,3-dihydroquinazolin-4(1H)-one;
3-(2-Methyl-6-oxo-1,6-dihydropyridin-3-yl)-1-((3S,4R)-3-methyltetrahydro-2H-pyran-4-yl)-7-(trifluoromethyl)-2,3-dihydroquinazolin-4(1H)-one;
rel-(R)-1-(4-fluoro-2-methylphenyl)-2-methyl-3-(6-oxo-1,6-dihydropyridin-3-yl)-7-(trifluoromethyl)-2,3-dihydroquinazolin-4(1H)-one;
rel-(R)-1-(4-fluoro-2-methylphenyl)-2-methyl-3-(6-oxo-1,6-dihydropyridin-3-yl)-7-(trifluoromethyl)-2,3-dihydroquinazolin-4(1H)-one;
1-(4-Fluoro-2-methylphenyl)-3-((2S,3S)-2-methyl-6-oxopiperidin-3-yl)-7-(trifluoromethyl)-2,3-dihydroquinazolin-4(1H)-one;
1-(4-Fluoro-2-methylphenyl)-3-((2R,3R)-2-methyl-6-oxopiperidin-3-yl)-7-(trifluoromethyl)-2,3-dihydroquinazolin-4(1H)-one;
3-(2,4-Dimethyl-6-oxo-1,6-dihydropyridin-3-yl)-1-(4-fluoro-2-isopropylphenyl)-7-(trifluoromethyl)-2,3-dihydroquinazolin-4(1H)-one;
3-(2,4-dimethyl-6-oxo-1,6-dihydropyridin-3-yl)-1-(4-fluoro-2-isopropylphenyl)-7-(trifluoromethyl)-2,3-dihydroquinazolin-4(1H)-one;
6-Chloro-3-(2,4-dimethyl-6-oxo-1,6-dihydropyridin-3-yl)-1-(4-fluoro-2-isopropylphenyl)-2,3-dihydroquinazolin-4(1H)-one;
6-Chloro-3-(2,4-dimethyl-6-oxo-1,6-dihydropyridin-3-yl)-1-(4-fluoro-2-isopropylphenyl)-2,3-dihydroquinazolin-4(1H)-one;
1-(2-(tert-Butyl)-4-fluorophenyl)-6-chloro-3-(2-methyl-6-oxo-1,6-dihydropyridin-3-yl)-2,3-dihydroquinazolin-4(1H)-one;
1-(2-(tert-Butyl)-4-fluorophenyl)-6-chloro-3-(2-methyl-6-oxo-1,6-dihydropyridin-3-yl)-2,3-dihydroquinazolin-4(1H)-one;
1-(4-Fluoro-2-methylphenyl)-3-(1-methyl-6-oxo-1,6-dihydropyridin-3-yl)-7-(trifluoromethyl)-2,3-dihydro quinazolin-4(1H)-one;
1-(4-Fluoro-2-methylphenyl)-3-(1-methyl-6-oxo-1,6-dihydropyridin-3-yl)-7-(trifluoromethyl)-2,3-dihydro quinazolin-4(1H)-one;
1-(4-fluoro-2-methylphenyl)-3-(2-methyl-6-oxo-1,6-dihydropyridin-3-yl)-6-(trifluoromethyl)-2,3-dihydroquinazolin-4(1H)-one;
1-(2-methyl-4-(trifluoromethoxy)phenyl)-3-(2-methyl-6-oxo-1,6-dihydropyridin-3-yl)-6-(trifluoromethyl)-2,3-dihydropyrido[2,3-d]pyrimidin-4(1H)-one; and
1-(4-fluoro-2-methylphenyl)-3-(2-methyl-6-oxo-1,6-dihydropyridin-3-yl)-6-(trifluoromethyl)-2,3-dihydropyrido[2,3-d]pyrimidin-4(1H)-one;
   or a pharmaceutically acceptable salt and/or a corresponding tautomer form thereof.

In another aspect, the present invention relates to a compound selected from:
1-(4-fluoro-2-methylphenyl)-3-(2-methyl-6-oxo-1,6-dihydropyridin-3-yl)-6-(trifluoromethyl)-2,3-dihydropyrido[2,3-d]pyrimidin-4(1H)-one;
1-(4-fluoro-2-methylphenyl)-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-3-(6-oxo-1,6-dihydropyridin-3-yl)-6-(trifluoromethyl)-2,3-dihydroquinazolin-4(1H)-one;
1-(2-methyl-4-(trifluoromethoxy)phenyl)-3-(2-methyl-6-oxo-1,6-dihydropyridin-3-yl)-6-(trifluoromethyl)-2,3-dihydropyrido[2,3-d]pyrimidin-4(1H)-one;
1-(4-bromo-2-methylphenyl)-3-(6-oxo-1,6-dihydropyridin-3-yl)-7-(trifluoromethyl)-2,3-dihydroquinazolin-4(1H)-one;
1-(4-fluorophenyl)-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-3-(2-methyl-6-oxo-1,6-dihydropyridin-3-yl)-6-(trifluoromethyl)-2,3-dihydroquinazolin-4(1H)-one; and
3-methyl-4-(3-(2-methyl-6-oxo-1,6-dihydropyridin-3-yl)-4-oxo-7-(trifluoromethyl)-3,4-dihydroquinazolin-1(2H)-yl)benzonitrile;
   or a pharmaceutically acceptable salt and/or a corresponding tautomer form thereof.

In another aspect, the present invention relates to a compound which is:

| Name | Structure |
|---|---|
| 1-(4-fluoro-2-methylphenyl)-3-(2-methyl- 6-oxo-1,6-dihydropyridin-3-yl)-7- (trifluoromethyl)-2,3-dihydroquinazolin- 4(1H)-one | 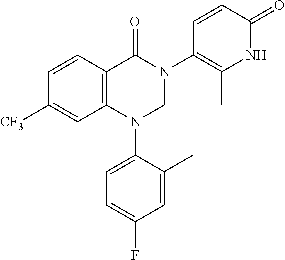 [SMILES: Cc1cc(F)ccc1N1CN(c2ccc(=O)[nH]c2C)C(=O)c2ccc(C(F)(F)F)cc21] |
| 1-(4-fluoro-2-methylphenyl)-3-(6-oxo-1,6- dihydropyridin-3-yl)-7-(trifluoromethyl)- 2,3-dihydroquinazolin-4(1H)-one | 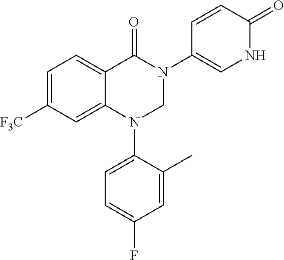 [SMILES: Cc1cc(F)ccc1N1CN(c2ccc(=O)[nH]c2)C(=O)c2ccc(C(F)(F)F)cc21] |
| 1-cyclohexyl-3-(6-oxo-1,6-dihydropyridin- 3-yl)-7-(trifluoromethyl)-2,3- dihydroquinazolin-4(1H)-one | 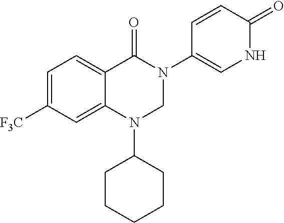 [SMILES: O=C1c2ccc(C(F)(F)F)cc2N(C2CCCCC2)CN1c1ccc(=O)[nH]c1] |
| 1-(4-fluoro-2,6-dimethylphenyl)-3-(6-oxo- 1,6-dihydropyridin-3-yl)-7- (trifluoromethyl)-2,3-dihydroquinazolin- 4(1H)-one | 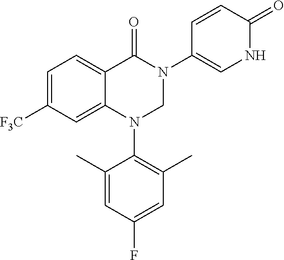 [SMILES: Cc1cc(F)cc(C)c1N1CN(c2ccc(=O)[nH]c2)C(=O)c2ccc(C(F)(F)F)cc21] |
| 1-(4-fluoro-2-methylphenyl)-2-methyl-3- (6-oxo-1,6-dihydropyridin-3-yl)-7-(trifluoro methyl)-2,3-dihydroquinazolin-4(1H)-one | 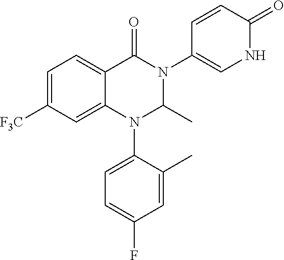 [SMILES: Cc1cc(F)ccc1N1c2cc(C(F)(F)F)ccc2C(=O)N(c2ccc(=O)[nH]c2)C1C] |
| 1-(2-chloro-4-fluorophenyl)-3-(6-oxo-1,6- dihydropyridin-3-yl)-7-(trifluoromethyl)- 2,3-dihydroquinazolin-4(1H)-one | 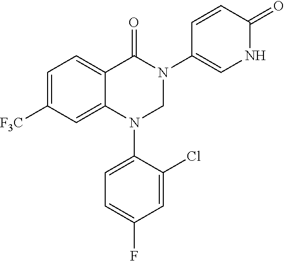 [SMILES: O=C1c2ccc(C(F)(F)F)cc2N(c2ccc(F)cc2Cl)CN1c1ccc(=O)[nH]c1] |
| 1-(4-fluoro-2-(2-hydroxyethoxy)phenyl)-3- (6-oxo-1,6-dihydropyridin-3-yl)-7- (trifluoromethyl)-2,3-dihydroquinazolin- 4(1H)-one | 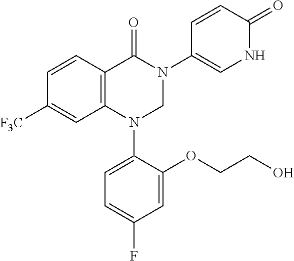 [SMILES: O=C1c2ccc(C(F)(F)F)cc2N(c2ccc(F)cc2OCCO)CN1c1ccc(=O)[nH]c1] |
| 1-(2,4-difluorophenyl)-3-(6-oxo-1,6- dihydropyridin-3-yl)-7-(trifluoromethyl)- 2,3-dihydroquinazolin-4(1H)-one | 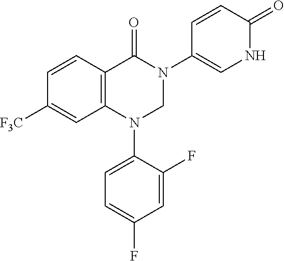 [SMILES: O=C1c2ccc(C(F)(F)F)cc2N(c2ccc(F)cc2F)CN1c1ccc(=O)[nH]c1] |
| 1-(2-ethyl-4-fluorophenyl)-3-(6-oxo-1,6- dihydropyridin-3-yl)-7-(trifluoromethyl)- 2,3-dihydroquinazolin-4(1H)-one | 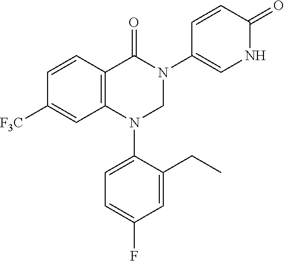 [SMILES: CCc1cc(F)ccc1N1CN(c2ccc(=O)[nH]c2)C(=O)c2ccc(C(F)(F)F)cc21] |
| 8-chloro-1-(4-fluoro-2-methylphenyl)-3-(6- oxo-1,6-dihydropyridin-3-yl)-7- (trifluoromethyl)-2,3-dihydroquinazolin- 4(1H)-one | 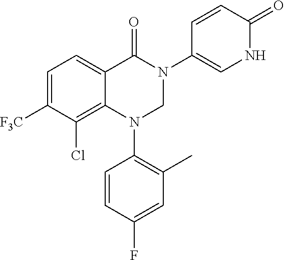 [SMILES: Cc1cc(F)ccc1N1CN(c2ccc(=O)[nH]c2)C(=O)c2ccc(C(F)(F)F)c(Cl)c21] |
| 1-(4-fluoro-2-methylphenyl)-8-methyl-3- (6-oxo-1,6-dihydropyridin-3-yl)-7- (trifluoromethyl)-2,3-dihydroquinazolin- 4(1H)-one | 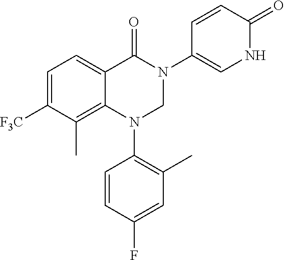 [SMILES: Cc1cc(F)ccc1N1CN(c2ccc(=O)[nH]c2)C(=O)c2ccc(C(F)(F)F)c(C)c21] |
| 3-(2-methyl-6-oxo-1,6-dihydropyridin-3- yl)-1-(2-methylpyridin-3-yl)-7- (trifluoromethyl)-2,3-dihydroquinazolin- 4(1H)-one | 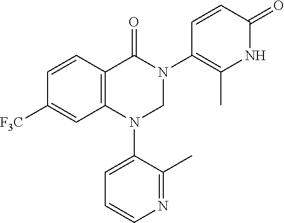 [SMILES: Cc1ncccc1N1CN(c2ccc(=O)[nH]c2C)C(=O)c2ccc(C(F)(F)F)cc21] |
| 1-(4-fluoro-2-isopropylphenyl)-3-(2- methyl-6-oxo-1,6-dihydropyridin-3-yl)-7- (trifluoromethyl)-2,3-dihydroquinazolin- 4(1H)-one | 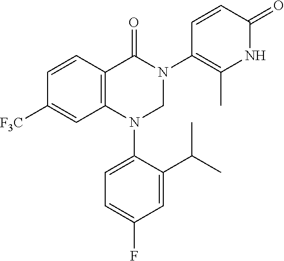 [SMILES: Cc1[nH]c(=O)ccc1N1CN(c2ccc(F)cc2C(C)C)c2cc(C(F)(F)F)ccc2C1=O] |
| 6-chloro-1-(4-fluoro-2-methylphenyl)-3-(2- methyl-6-oxo-1,6-dihydropyridin-3-yl)-2,3- dihydroquinazolin-4(1H)-one | 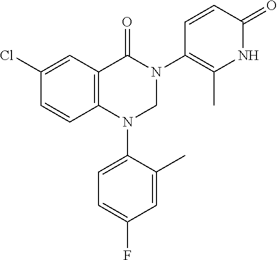 [SMILES: Cc1cc(F)ccc1N1CN(c2ccc(=O)[nH]c2C)C(=O)c2cc(Cl)ccc21] |
| 1-(4-fluorophenyl)-3-(2-methyl-6-oxo-1,6- dihydropyridin-3-yl)-2,3- dihydroquinazolin-4(1H)-one | 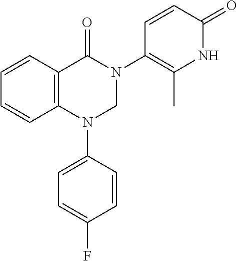 [SMILES: Cc1[nH]c(=O)ccc1N1CN(c2ccc(F)cc2)c2ccccc2C1=O] |
| 3-(2-methyl-6-oxo-1,6-dihydropyridin-3- yl)-1-(o-tolyl)-2,3-dihydroquinazolin- 4(1H)-one | 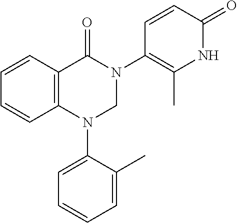 [SMILES: Cc1ccccc1N1CN(c2ccc(=O)[nH]c2C)C(=O)c2ccccc21] |
| 1-(4-fluoro-2-methylphenyl)-6-methyl-3- (2-methyl-6-oxo-1,6-dihydropyridin-3-yl)- 2,3-dihydroquinazolin-4(1H)-one | 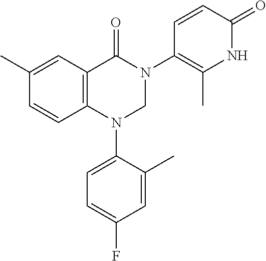 [SMILES: Cc1ccc2c(c1)C(=O)N(c1ccc(=O)[nH]c1C)CN2c1ccc(F)cc1C] |
| 1-(4-fluoro-2-methylphenyl)-3-(2-methyl- 6-oxo-1,6-dihydropyridin-3-yl)-2,3- dihydroquinazolin-4(1H)-one | 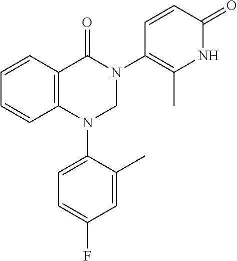 [SMILES: Cc1cc(F)ccc1N1CN(c2ccc(=O)[nH]c2C)C(=O)c2ccccc21] |
| 1-(4-fluoro-2-methylphenyl)-3-(6-methyl- 2-oxo-1,2-dihydropyridin-4-yl)- 7-(trifluoromethyl)-2,3- dihydroquinazolin-4(1H)-one | 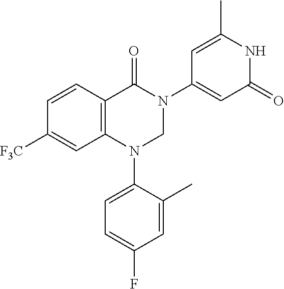 [SMILES: Cc1cc(N2CN(c3ccc(F)cc3C)c3cc(C(F)(F)F)ccc3C2=O)cc(=O)[nH]1] |
| 1-(4-fluoro-2-methylphenyl)-3-(5-methyl- 2-oxo-1,2-dihydropyridin-4-yl)- 7-(trifluoromethyl)-2,3- dihydroquinazolin-4(1H)-one | 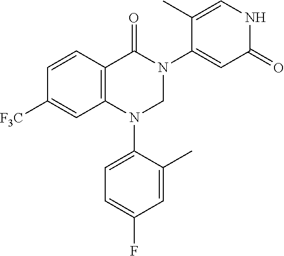 [SMILES: Cc1c[nH]c(=O)cc1N1CN(c2ccc(F)cc2C)c2cc(C(F)(F)F)ccc2C1=O] |
| 1-(4-fluoro-2-methylphenyl)-3-(3-methyl- 2-oxo-1,2-dihydropyridin-4-yl)-7- (trifluoromethyl)-2,3-dihydroquinazolin- 4(1H)-one | 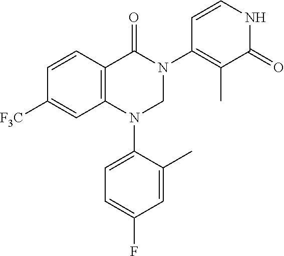 [SMILES: Cc1cc(F)ccc1N1CN(c2cc[nH]c(=O)c2C)C(=O)c2ccc(C(F)(F)F)cc21] |
| 1-(4-fluorophenyl)-3-(6-oxo-1,6- dihydropyridin-3-yl)-7-(trifluoromethyl)- 2,3-dihydroquinazolin-4(1H)-one | 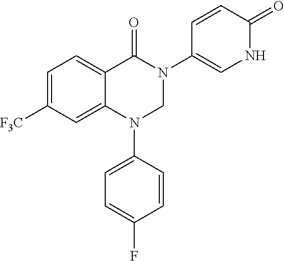 [SMILES: O=C1c2ccc(C(F)(F)F)cc2N(c2ccc(F)cc2)CN1c1ccc(=O)[nH]c1] |
| 1-(4-fluoro-2-methylphenyl)-3-(2-oxo-1,2- dihydropyrimidin-5-yl)-7-(trifluoromethyl)- 2,3-dihydroquinazolin-4(1H)-one | 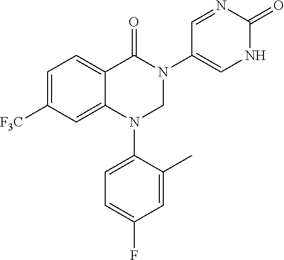 [SMILES: Cc1cc(F)ccc1N1CN(c2cnc(=O)[nH]c2)C(=O)c2ccc(C(F)(F)F)cc21] |
| 1-(4-bromo-2-methylphenyl)-3-(6-oxo-1,6- dihydropyridin-3-yl)-7-(trifluoromethyl)- 2,3-dihydroquinazolin-4(1H)-one | 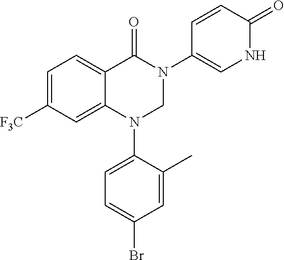 [SMILES: Cc1cc(Br)ccc1N1CN(c2ccc(=O)[nH]c2)C(=O)c2ccc(C(F)(F)F)cc21] |
| 1-(4-fluorophenyl)-3-(2-methyl-6-oxo-1,6- dihydropyridin-3-yl)-7-(trifluoromethyl)- 2,3-dihydroquinazolin-4(1H)-one | 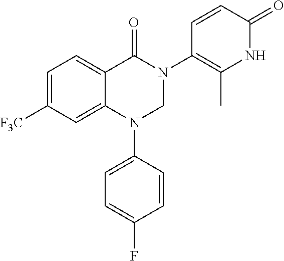 [SMILES: Cc1[nH]c(=O)ccc1N1CN(c2ccc(F)cc2)c2cc(C(F)(F)F)ccc2C1=O] |
| 3-methyl-4-(3-(2-methyl-6-oxo-1,6- dihydropyridin-3-yl)-4-oxo-7- (trifluoromethyl)-3,4-dihydroquinazolin- 1(2H)-yl)benzonitrile | 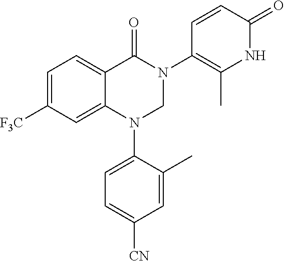 [SMILES: Cc1cc(C#N)ccc1N1CN(c2ccc(=O)[nH]c2C)C(=O)c2ccc(C(F)(F)F)cc21] |
| 3-(2-ethyl-6-oxo-1,6-dihydropyridin-3-yl)- 1-(4-fluoro-2-methylphenyl)-7- (trifluoromethyl)-2,3-dihydroquinazolin- 4(1H)-one | 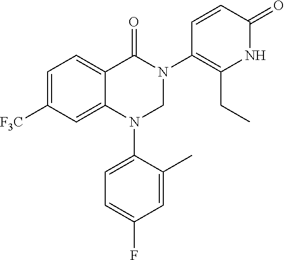 [SMILES: CCc1[nH]c(=O)ccc1N1CN(c2ccc(F)cc2C)c2cc(C(F)(F)F)ccc2C1=O] |
| 3-(2-chloro-6-oxo-1,6-dihydropyridin-3-yl)- 1-(4-fluoro-2-methylphenyl)-7- (trifluoromethyl)-2,3-dihydroquinazolin- 4(1H)-one | 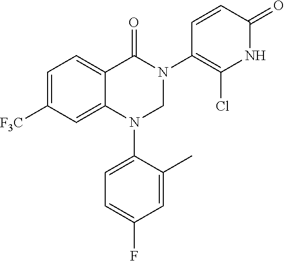 [SMILES: Cc1cc(F)ccc1N1CN(c2ccc(=O)[nH]c2Cl)C(=O)c2ccc(C(F)(F)F)cc21] |
| 7-bromo-1-(4-fluoro-2-methylphenyl)-3-(2- methyl-6-oxo-1,6-dihydropyridin-3-yl)-2,3- dihydroquinazolin-4(1H)-one | 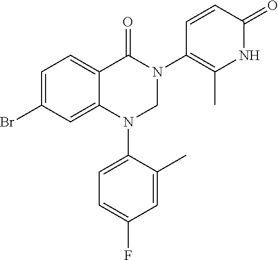 [SMILES: Cc1cc(F)ccc1N1CN(c2ccc(=O)[nH]c2C)C(=O)c2ccc(Br)cc21] |
| 1-(4-fluoro-2-methylphenyl)-3-(2-methyl- 6-oxo-1,6-dihydropyridin-3-yl)-4-oxo- 1,2,3,4-tetrahydroquinazoline-7- carbonitrile | 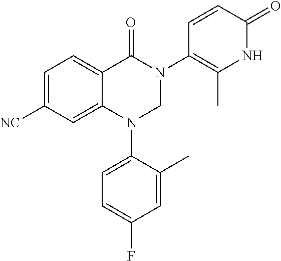 [SMILES: Cc1cc(F)ccc1N1CN(c2ccc(=O)[nH]c2C)C(=O)c2ccc(C#N)cc21] |
| 3-(1-(4-fluoro-2-methylphenyl)-4-oxo-7- (trifluoromethyl)-1,4-dihydroquinazolin- 3(2H)-yl)-6-oxo-1,6-dihydropyridine-2- carbonitrile | 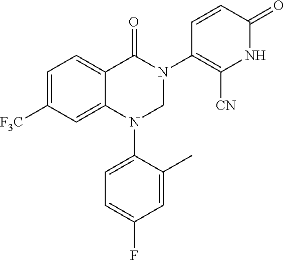 [SMILES: Cc1cc(F)ccc1N1CN(c2ccc(=O)[nH]c2C#N)C(=O)c2ccc(C(F)(F)F)cc21] |
| 1-(2,4-difluorophenyl)-3-(2-methyl-6-oxo- 1,6-dihydropyridin-3-yl)-7- (trifluoromethyl)-2,3-dihydroquinazolin- 4(1H)-one | 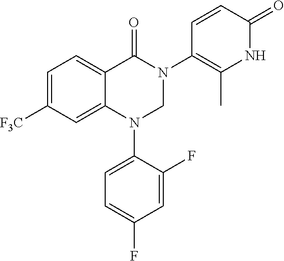 [SMILES: Cc1[nH]c(=O)ccc1N1CN(c2ccc(F)cc2F)c2cc(C(F)(F)F)ccc2C1=O] |
| 1-(4-ethoxyphenyl)-3-(2-methyl-6-oxo-1,6- dihydropyridin-3-yl)-7-(trifluoromethyl)- 2,3-dihydroquinazolin-4(1H)-one | 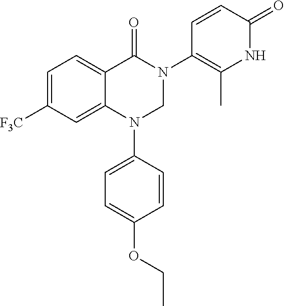 [SMILES: CCOc1ccc(N2CN(c3ccc(=O)[nH]c3C)C(=O)c3ccc(C(F)(F)F)cc32)cc1] |
| 1-(4-fluoro-2-methylphenyl)-3-(2-oxo-1,2- dihydropyridin-4-yl)-7-(trifluoromethyl)- 2,3-dihydroquinazolin-4(1H)-one | 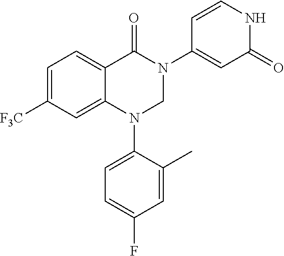 [SMILES: Cc1cc(F)ccc1N1CN(c2cc[nH]c(=O)c2)C(=O)c2ccc(C(F)(F)F)cc21] |
| 1-(4-fluoro-2-methylphenyl)-3-(6-oxo-1,6- dihydropyridin-3-yl)-6-(trifluoromethyl)- 2,3-dihydroquinazolin-4(1H)-one | 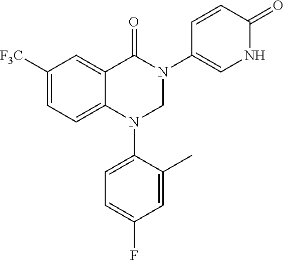 [SMILES: Cc1cc(F)ccc1N1CN(c2ccc(=O)[nH]c2)C(=O)c2cc(C(F)(F)F)ccc21] |
| 1-(4-fluoro-2-methoxyphenyl)-3-(6-oxo- 1,6-dihydropyridin-3-yl)-7- (trifluoromethyl)-2,3-dihydroquinazolin- 4(1H)-one | 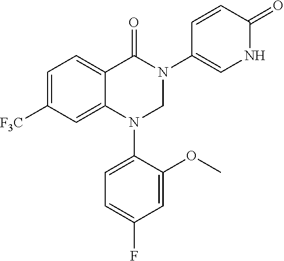 [SMILES: COc1cc(F)ccc1N1CN(c2ccc(=O)[nH]c2)C(=O)c2ccc(C(F)(F)F)cc21] |
| 1-(4-fluoro-2-methylphenyl)-3-(6-oxo-1,6- dihydropyridin-3-yl)-5-(trifluoromethyl)- 2,3-dihydroquinazolin-4(1H)-one | 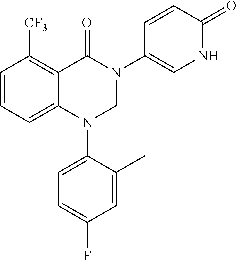 [SMILES: Cc1cc(F)ccc1N1CN(c2ccc(=O)[nH]c2)C(=O)c2c1cccc2C(F)(F)F] |
| 1-(4-fluoro-2-methylphenyl)-3-(5-methyl- 6-oxo-1,6-dihydropyridin-3-yl)-7- (trifluoromethyl)-2,3-dihydroquinazolin- 4(1H)-one | 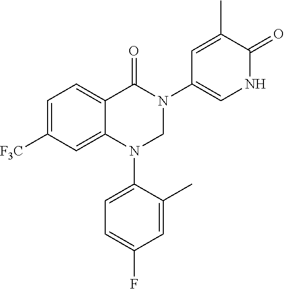 [SMILES: Cc1cc(F)ccc1N1CN(c2c[nH]c(=O)c(C)c2)C(=O)c2ccc(C(F)(F)F)cc21] |
| 1-(4-fluoro-2-methylphenyl)-3-(6-oxo-1,6- dihydropyridazin-3-yl)-7-(trifluoromethyl)- 2,3-dihydroquinazolin-4(1H)-one | 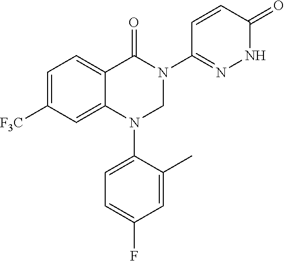 [SMILES: Cc1cc(F)ccc1N1CN(c2ccc(=O)[nH]n2)C(=O)c2ccc(C(F)(F)F)cc21] |
| 1-(4-fluorobenzyl)-3-(6-oxo-1,6- dihydropyridin-3-yl)-7-(trifluoromethyl)- 2,3-dihydroquinazolin-4(1H)-one | 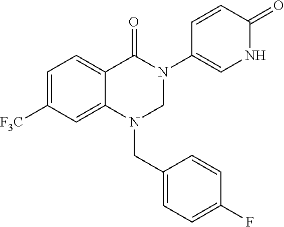 [SMILES: O=C1c2ccc(C(F)(F)F)cc2N(Cc2ccc(F)cc2)CN1c1ccc(=O)[nH]c1] |
| 1-(4-fluoro-2-methylphenyl)-3-(4-methyl- 6-oxo-1,6-dihydropyridin-3-yl)-7- (trifluoromethyl)-2,3-dihydroquinazolin- 4(1H)-one | 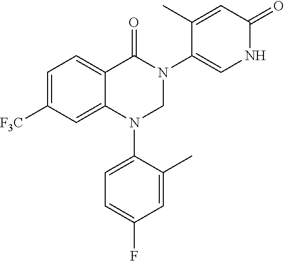 [SMILES: Cc1cc(=O)[nH]cc1N1CN(c2ccc(F)cc2C)c2cc(C(F)(F)F)ccc2C1=O] |
| 1-(4-fluoro-2-methylphenyl)-4-oxo-3-(6- oxo-1,6-dihydropyridin-3-yl)-1,2,3,4- tetrahydroquinazoline-7-carbonitrile | 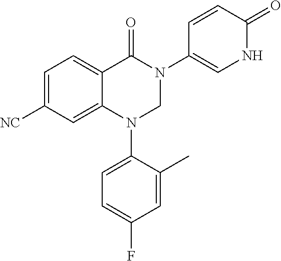 [SMILES: Cc1cc(F)ccc1N1CN(c2ccc(=O)[nH]c2)C(=O)c2ccc(C#N)cc21] |
| 1-(4-fluoro-2-methoxyphenyl)-3-(6-oxo- 1,6-dihydropyridin-3-yl)-5- (trifluoromethyl)-2,3-dihydroquinazolin- 4(1H)-one | 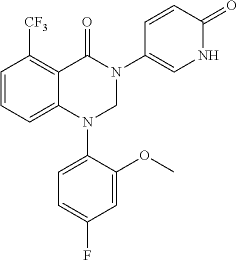 [SMILES: COc1cc(F)ccc1N1CN(c2ccc(=O)[nH]c2)C(=O)c2c1cccc2C(F)(F)F] |
| 1-(4-fluoro-2-methylphenyl)-3-(1-methyl- 6-oxo-1,6-dihydropyridin-3-yl)-7- (trifluoromethyl)-2,3-dihydroquinazolin- 4(1H)-one | 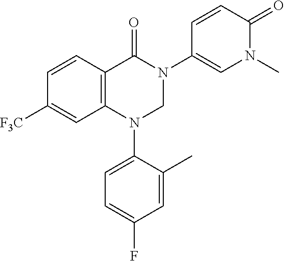 [SMILES: Cc1cc(F)ccc1N1CN(c2ccc(=O)n(C)c2)C(=O)c2ccc(C(F)(F)F)cc21] |
| 1-(4-fluoro-2-methylphenyl)-3-(2-methyl- 6-oxo-1,6-dihydropyridin-3-yl)-7- (trifluoromethoxy)-2,3-dihydroquinazolin- 4(1H)-one | 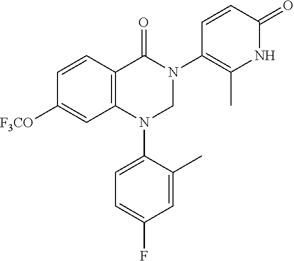 [SMILES: Cc1cc(F)ccc1N1CN(c2ccc(=O)[nH]c2C)C(=O)c2ccc(OC(F)(F)F)cc21] |
| 1-(4-fluoro-2-methylphenyl)-7-methyl-3- (2-methyl-6-oxo-1,6-dihydropyridin-3-yl)- 2,3-dihydroquinazolin-4(1H)-one | 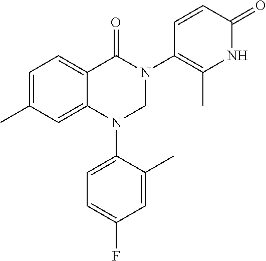 [SMILES: Cc1ccc2c(c1)N(c1ccc(F)cc1C)CN(c1ccc(=O)[nH]c1C)C2=O] |
| 1-(4-fluoro-2-methylphenyl)-3-(2-methyl- 6-oxo-1,6-dihydropyridin-3-yl)-6- (trifluoromethyl)-2,3-dihydroquinazolin- 4(1H)-one | 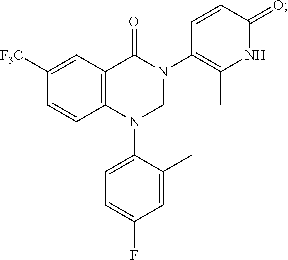 [SMILES: Cc1cc(F)ccc1N1CN(c2ccc(=O)[nH]c2C)C(=O)c2cc(C(F)(F)F)ccc21] |

or a pharmaceutically acceptable salt and/or a corresponding tautomer form thereof.

In another aspect, the present invention relates to a compound of Formula (IIIA):

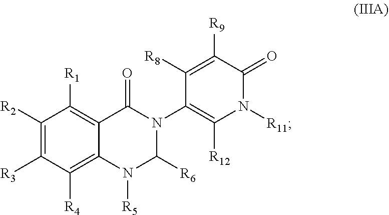

where:

R¹ or R⁴ is -hydrogen, -halogen, -straight or branched-(C₁₋₆)-alkyl or -straight or branched-(C₁₋₆)-haloalkyl;

R² or R³ is -hydrogen, -halogen, —C≡N, —OH, —NHR^{a}, —NR^{a}R^{b}, -straight or branched-(C₁₋₆)-alkyl, —(CF₂)ₙ(CH₂)ₒOH, —OR^{c} or —S(O)ₚR^{d};

where:
R¹, R², R³ or R⁴ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R⁵ is an unsaturated or saturated carbocyclic ring, —CH₂-unsaturated carbocyclic ring, unsaturated or saturated heterocyclic or heteroaryl ring;

where:
the unsaturated or saturated heterocyclic ring of R⁵ or the heteroaryl ring of R⁵, respectively, contains at least one heteroatom selected from nitrogen, oxygen or sulfur;
where:
   R⁵ optionally is substituted with hydrogen, halogen, —C≡N, —NHR^{a}, —NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;

R⁶ is -hydrogen, -straight or branched-(C₁₋₆)-alkyl or -straight or branched-(C₁₋₆)-haloalkyl;

R⁸, R⁹ or R¹² is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;

R¹¹ is -hydrogen or -straight or branched (C₁₋₆) alkyl;
where:
   each R^{a}, R^{b} or R^{c} of R¹, R², R³, R⁴, R⁸, R⁹, R¹⁰, R¹¹ or R¹² as defined above is hydrogen, -straight or branched-(C₁₋₆)-alkyl, -straight or branched-(C₁₋₆)-haloalkyl or —(C₃₋₆)-cycloalkyl;
   where:
      R^{a} optionally further is substituted with —OH;
      R^{d} is -hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
      where:
         each R^{a} or R^{b} of NHR^{a} or NR^{a}R^{b} as defined in R^{d} respectively, is -hydrogen, -straight or branched-(C₁₋₆)-alkyl, -straight or branched-(C₁₋₆)-haloalkyl or —(C₃₋₆)-cycloalkyl;

n, o or p is 0 or an integer from 1 to 5; or

a pharmaceutically acceptable salt and/or a corresponding tautomer form thereof.

In another aspect, the present invention relates to a compound which is:

| Name | Structure |
|---|---|
| 1-(4-fluoro-2-methylphenyl)-3-(2-methyl- 6-oxo-1,6-dihydropyridin-3-yl)-7- (trifluoromethyl)-2,3-dihydroquinazolin- 4(1H)-one | 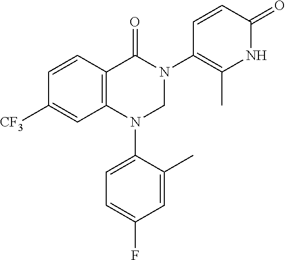 [SMILES: Cc1cc(F)ccc1N1CN(c2ccc(=O)[nH]c2C)C(=O)c2ccc(C(F)(F)F)cc21] |
| 1-(4-fluoro-2-methylphenyl)-3-(6-oxo- 1,6-dihydropyridin-3-yl)-7- (trifluoromethyl)-2,3-dihydroquinazolin- 4(1H)-one | 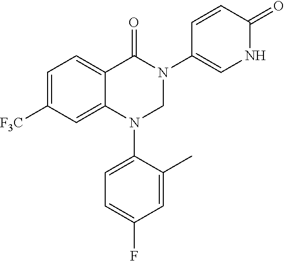 [SMILES: Cc1cc(F)ccc1N1CN(c2ccc(=O)[nH]c2)C(=O)c2ccc(C(F)(F)F)cc21] |
| 1-cyclohexyl-3-(6-oxo-1,6- dihydropyridin-3-yl)-7-(trifluoromethyl)- 2,3-dihydroquinazolin-4(1H)-one | 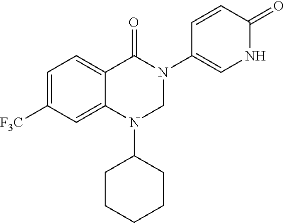 [SMILES: O=C1c2ccc(C(F)(F)F)cc2N(C2CCCCC2)CN1c1ccc(=O)[nH]c1] |
| 1-(4-fluoro-2,6-dimethylphenyl)-3-(6- oxo-1,6-dihydropyridin-3-yl)-7- (trifluoromethyl)-2,3-dihydroquinazolin- 4(1H)-one | 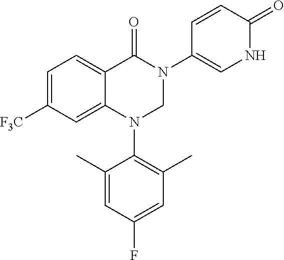 [SMILES: Cc1cc(F)cc(C)c1N1CN(c2ccc(=O)[nH]c2)C(=O)c2ccc(C(F)(F)F)cc21] |
| 1-(4-fluoro-2-methylphenyl)-2-methyl-3- (6-oxo-1,6-dihydropyridin-3-yl)-7- (trifluoromethyl)-2,3-dihydroquinazolin- 4(1H)-one | 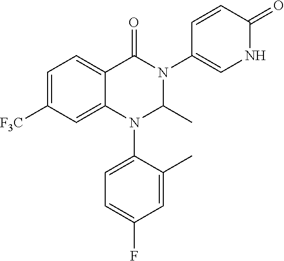 [SMILES: Cc1cc(F)ccc1N1c2cc(C(F)(F)F)ccc2C(=O)N(c2ccc(=O)[nH]c2)C1C] |
| 1-(2-chloro-4-fluorophenyl)-3-(6-oxo- 1,6-dihydropyridin-3-yl)-7- (trifluoromethyl)-2,3-dihydroquinazolin- 4(1H)-one | 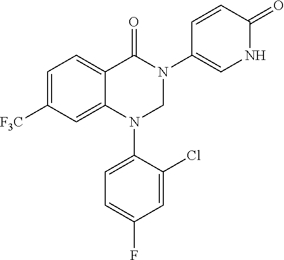 [SMILES: O=C1c2ccc(C(F)(F)F)cc2N(c2ccc(F)cc2Cl)CN1c1ccc(=O)[nH]c1] |
| 1-(4-fluoro-2-(2-hydroxyethoxy) phenyl)- 3-(6-oxo-1,6-dihydropyridin-3-yl)-7- (trifluoromethyl)-2,3-dihydroquinazolin- 4(1H)-one | 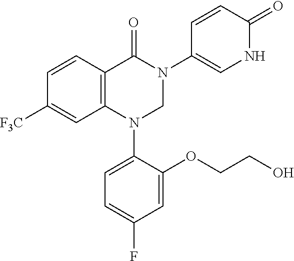 [SMILES: O=C1c2ccc(C(F)(F)F)cc2N(c2ccc(F)cc2OCCO)CN1c1ccc(=O)[nH]c1] |
| 1-(2,4-difluorophenyl)-3-(6-oxo-1,6- dihydropyridin-3-yl)-7-(trifluoromethyl)- 2,3-dihydroquinazolin-4(1H)-one | 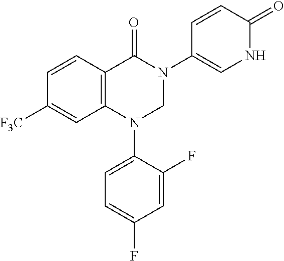 [SMILES: O=C1c2ccc(C(F)(F)F)cc2N(c2ccc(F)cc2F)CN1c1ccc(=O)[nH]c1] |
| 1-(2-ethyl-4-fluorophenyl)-3-(6-oxo-1,6- dihydropyridin-3-yl)-7-(trifluoromethyl)- 2,3-dihydroquinazolin-4(1H)-one | 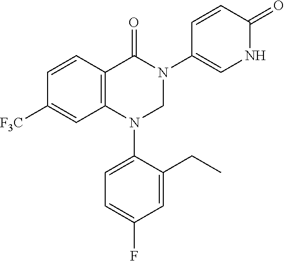 [SMILES: CCc1cc(F)ccc1N1CN(c2ccc(=O)[nH]c2)C(=O)c2ccc(C(F)(F)F)cc21] |
| 8-chloro-1-(4-fluoro-2-methylphenyl)-3- (6-oxo-1,6-dihydropyridin-3-yl)-7- (trifluoromethyl)-2,3-dihydroquinazolin- 4(1H)-one | 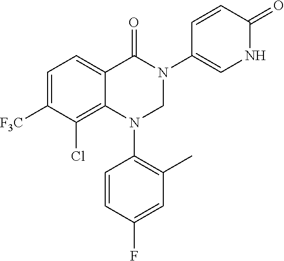 [SMILES: Cc1cc(F)ccc1N1CN(c2ccc(=O)[nH]c2)C(=O)c2ccc(C(F)(F)F)c(Cl)c21] |
| 1-(4-fluoro-2-methylphenyl)-8-methyl-3- (6-oxo-1,6-dihydropyridin-3-yl)-7- (trifluoromethyl)-2,3-dihydroquinazolin- 4(1H)-one | 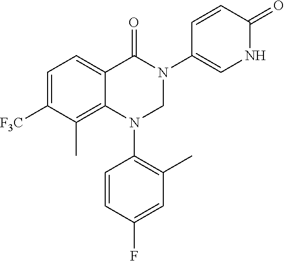 [SMILES: Cc1cc(F)ccc1N1CN(c2ccc(=O)[nH]c2)C(=O)c2ccc(C(F)(F)F)c(C)c21] |
| 3-(2-methyl-6-oxo-1,6-dihydropyridin-3- yl)-1-(2-methylpyridin-3-yl)-7- (trifluoromethyl)-2,3-dihydroquinazolin- 4(1H)-one | 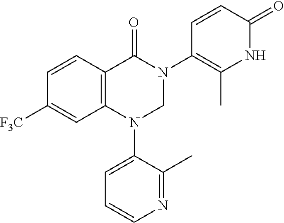 [SMILES: Cc1ncccc1N1CN(c2ccc(=O)[nH]c2C)C(=O)c2ccc(C(F)(F)F)cc21] |
| 1-(4-fluoro-2-isopropylphenyl)-3-(2- methyl-6-oxo-1,6-dihydropyridin-3-yl)-7- (trifluoromethyl)-2,3-dihydroquinazolin- 4(1H)-one | 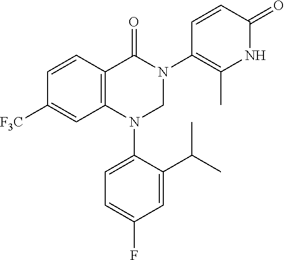 [SMILES: Cc1[nH]c(=O)ccc1N1CN(c2ccc(F)cc2C(C)C)c2cc(C(F)(F)F)ccc2C1=O] |
| 6-chloro-1-(4-fluoro-2-methylphenyl)-3- (2-methyl-6-oxo-1,6-dihydropyridin-3- yl)-2,3-dihydroquinazolin-4(1H)-one | 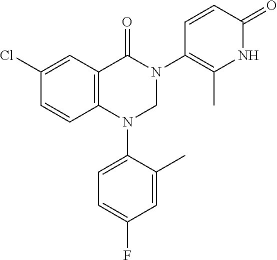 [SMILES: Cc1cc(F)ccc1N1CN(c2ccc(=O)[nH]c2C)C(=O)c2cc(Cl)ccc21] |
| 1-(4-fluorophenyl)-3-(2-methyl-6-oxo- 1,6-dihydropyridin-3-yl)-2,3- dihydroquinazolin-4(1H)-one | 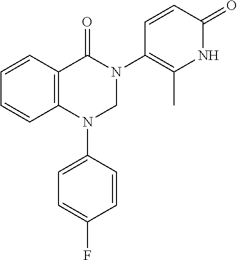 [SMILES: Cc1[nH]c(=O)ccc1N1CN(c2ccc(F)cc2)c2ccccc2C1=O] |
| 3-(2-methyl-6-oxo-1,6-dihydropyridin-3- yl)-1-(o-tolyl)-2,3-dihydroquinazolin- 4(1H)-one | 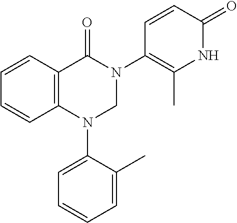 [SMILES: Cc1ccccc1N1CN(c2ccc(=O)[nH]c2C)C(=O)c2ccccc21] |
| 1-(4-fluoro-2-methylphenyl)-6-methyl-3- (2-methyl-6-oxo-1,6-dihydropyridin-3- yl)-2,3-dihydroquinazolin-4(1H)-one | 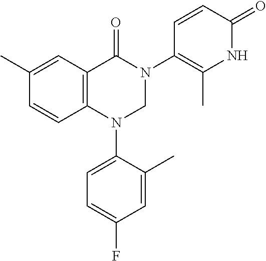 [SMILES: Cc1ccc2c(c1)C(=O)N(c1ccc(=O)[nH]c1C)CN2c1ccc(F)cc1C] |
| 1-(4-fluoro-2-methylphenyl)-3-(2-methyl- 6-oxo-1,6-dihydropyridin-3-yl)-2,3- dihydroquinazolin-4(1H)-one | 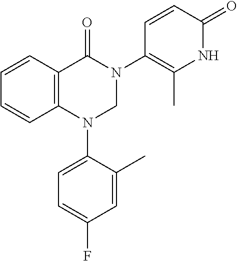 [SMILES: Cc1cc(F)ccc1N1CN(c2ccc(=O)[nH]c2C)C(=O)c2ccccc21] |
| 1-(4-fluorophenyl)-3-(6-oxo-1,6- dihydropyridin-3-yl)-7-(trifluoromethyl)- 2,3-dihydroquinazolin-4(1H)-one | 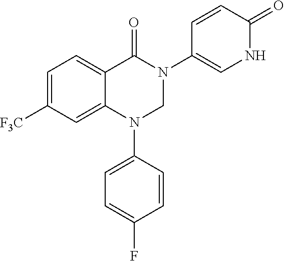 [SMILES: O=C1c2ccc(C(F)(F)F)cc2N(c2ccc(F)cc2)CN1c1ccc(=O)[nH]c1] |
| 1-(4-bromo-2-methylphenyl)-3-(6-oxo- 1,6-dihydropyridin-3-yl)- 7-(trifluoromethyl)-2,3- dihydroquinazolin-4(1H)- one | 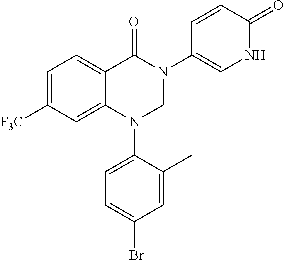 [SMILES: Cc1cc(Br)ccc1N1CN(c2ccc(=O)[nH]c2)C(=O)c2ccc(C(F)(F)F)cc21] |
| 1-(4-fluorophenyl)-3-(2-methyl-6-oxo- 1,6-dihydropyridin-3-yl)- 7-(trifluoromethyl)-2,3- dihydroquinazolin-4(1H)- one | 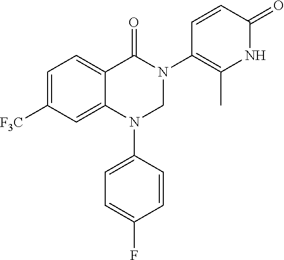 [SMILES: Cc1[nH]c(=O)ccc1N1CN(c2ccc(F)cc2)c2cc(C(F)(F)F)ccc2C1=O] |
| 3-methyl-4-(3-(2-methyl-6-oxo-1,6- dihydropyridin-3-yl)-4-oxo-7- (trifluoromethyl)-3,4-dihydroquinazolin- 1(2H)-yl)benzonitrile | 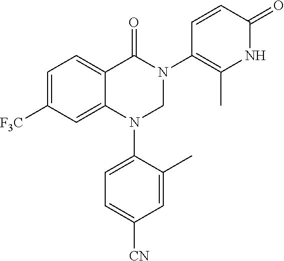 [SMILES: Cc1cc(C#N)ccc1N1CN(c2ccc(=O)[nH]c2C)C(=O)c2ccc(C(F)(F)F)cc21] |
| 3-(2-ethyl-6-oxo-1,6-dihydropyridin-3- yl)-1-(4-fluoro-2-methylphenyl)-7- (trifluoromethyl)-2,3-dihydroquinazolin- 4(1H)-one | 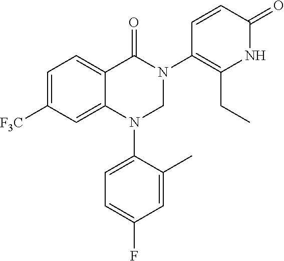 [SMILES: CCc1[nH]c(=O)ccc1N1CN(c2ccc(F)cc2C)c2cc(C(F)(F)F)ccc2C1=O] |
| 3-(2-chloro-6-oxo-1,6-dihydropyridin-3- yl)-1-(4-fluoro-2-methylphenyl)-7- (trifluoromethyl)-2,3-dihydroquinazolin- 4(1H)-one | 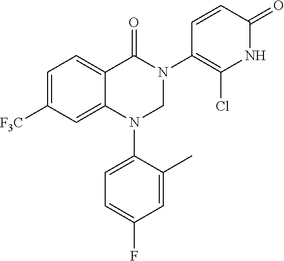 [SMILES: Cc1cc(F)ccc1N1CN(c2ccc(=O)[nH]c2Cl)C(=O)c2ccc(C(F)(F)F)cc21] |
| 7-bromo-1-(4-fluoro-2-methylphenyl)-3- (2-methyl-6-oxo-1,6-dihydropyridin-3- yl)-2,3-dihydroquinazolin-4(1H)-one | 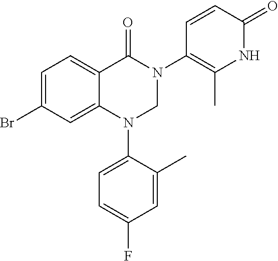 [SMILES: Cc1cc(F)ccc1N1CN(c2ccc(=O)[nH]c2C)C(=O)c2ccc(Br)cc21] |
| 1-(4-fluoro-2-methylphenyl)-3-(2-methyl- 6-oxo-1,6-dihydropyridin-3-yl)-4-oxo- 1,2,3,4-tetrahydroquinazoline-7- carbonitrile | 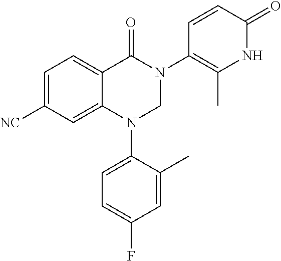 [SMILES: Cc1cc(F)ccc1N1CN(c2ccc(=O)[nH]c2C)C(=O)c2ccc(C#N)cc21] |
| 3-(1-(4-fluoro-2-methylphenyl)-4-oxo-7- (trifluoromethyl)-1,4-dihydroquinazolin- 3(2H)-yl)-6-oxo-1,6-dihydropyridine-2- carbonitrile | 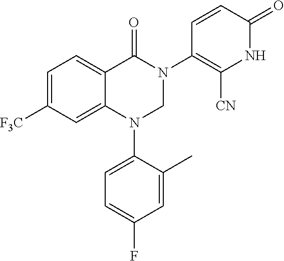 [SMILES: Cc1cc(F)ccc1N1CN(c2ccc(=O)[nH]c2C#N)C(=O)c2ccc(C(F)(F)F)cc21] |
| 1-(2,4-difluorophenyl)-3-(2-methyl-6- oxo-1,6-dihydropyridin-3-yl)-7- (trifluoromethyl)-2,3-dihydroquinazolin- 4(1H)-one | 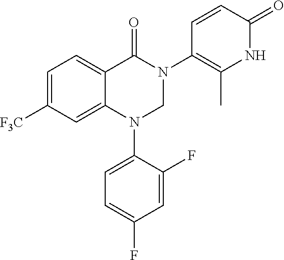 [SMILES: Cc1[nH]c(=O)ccc1N1CN(c2ccc(F)cc2F)c2cc(C(F)(F)F)ccc2C1=O] |
| 1-(4-ethoxyphenyl)-3-(2-methyl-6-oxo- 1,6-dihydropyridin-3-yl)- 7-(trifluoromethyl)-2,3- dihydroquinazolin-4(1H)- one | 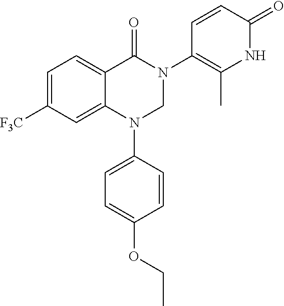 [SMILES: CCOc1ccc(N2CN(c3ccc(=O)[nH]c3C)C(=O)c3ccc(C(F)(F)F)cc32)cc1] |
| 1-(4-fluoro-2-methylphenyl)-3-(6-oxo- 1,6-dihydropyridin-3-yl)- 6-(trifluoromethyl)-2,3- dihydroquinazolin-4(1H)- one | 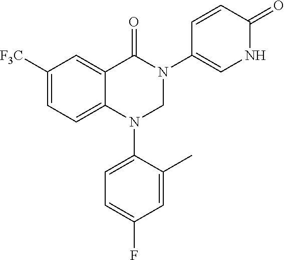 [SMILES: Cc1cc(F)ccc1N1CN(c2ccc(=O)[nH]c2)C(=O)c2cc(C(F)(F)F)ccc21] |
| 1-(4-fluoro-2-methoxyphenyl)-3-(6-oxo- 1,6-dihydropyridin-3-yl)-7- (trifluoromethyl)-2,3-dihydroquinazolin- 4(1H)-one | 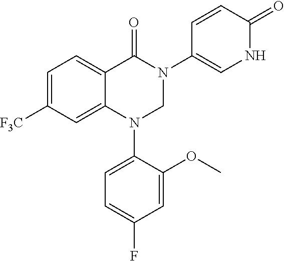 [SMILES: COc1cc(F)ccc1N1CN(c2ccc(=O)[nH]c2)C(=O)c2ccc(C(F)(F)F)cc21] |
| 1-(4-fluoro-2-methylphenyl)-3-(6-oxo- 1,6-dihydropyridin-3-yl)- 5-(trifluoromethyl)-2,3- dihydroquinazolin-4(1H)- one | 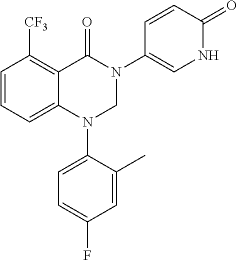 [SMILES: Cc1cc(F)ccc1N1CN(c2ccc(=O)[nH]c2)C(=O)c2c1cccc2C(F)(F)F] |
| 1-(4-fluoro-2-methylphenyl)-3-(5-methyl- 6-oxo-1,6-dihydropyridin-3-yl)-7- (trifluoromethyl)-2,3-dihydroquinazolin- 4(1H)-one | 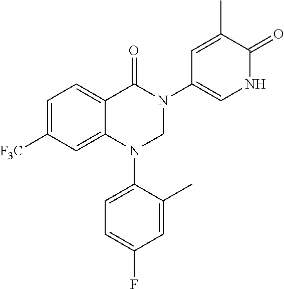 [SMILES: Cc1cc(F)ccc1N1CN(c2c[nH]c(=O)c(C)c2)C(=O)c2ccc(C(F)(F)F)cc21] |
| 1-(4-fluorobenzyl)-3-(6-oxo-1,6- dihydropyridin-3-yl)-7-(trifluoromethyl)- 2,3-dihydroquinazolin-4(1H)-one | 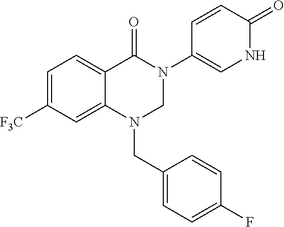 [SMILES: O=C1c2ccc(C(F)(F)F)cc2N(Cc2ccc(F)cc2)CN1c1ccc(=O)[nH]c1] |
| 1-(4-fluoro-2-methylphenyl)-3-(4-methyl- 6-oxo-1,6-dihydropyridin-3-yl)-7- (trifluoromethyl)-2,3-dihydroquinazolin- 4(1H)-one | 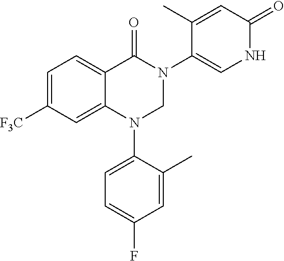 [SMILES: Cc1cc(=O)[nH]cc1N1CN(c2ccc(F)cc2C)c2cc(C(F)(F)F)ccc2C1=O] |
| 1-(4-fluoro-2-methylphenyl)-4-oxo-3-(6- oxo-1,6-dihydropyridin-3-yl)-1,2,3,4- tetrahydroquinazoline-7-carbonitrile | 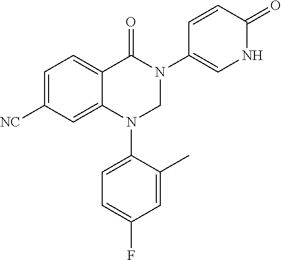 [SMILES: Cc1cc(F)ccc1N1CN(c2ccc(=O)[nH]c2)C(=O)c2ccc(C#N)cc21] |
| 1-(4-fluoro-2-methoxyphenyl)-3-(6-oxo- 1,6-dihydropyridin-3-yl)-5- (trifluoromethyl)-2,3-dihydroquinazolin- 4(1H)-one | 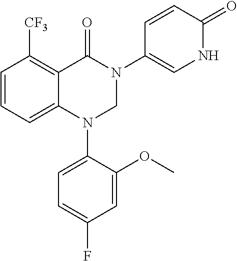 [SMILES: COc1cc(F)ccc1N1CN(c2ccc(=O)[nH]c2)C(=O)c2c1cccc2C(F)(F)F] |
| 1-(4-fluoro-2-methylphenyl)-3-(1-methyl- 6-oxo-1,6-dihydropyridin-3-yl)-7- (trifluoromethyl)-2,3-dihydroquinazolin- 4(1H)-one | 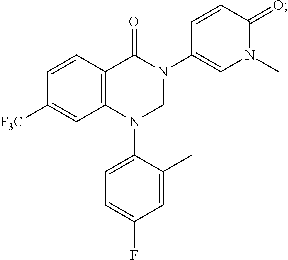 [SMILES: Cc1cc(F)ccc1N1CN(c2ccc(=O)n(C)c2)C(=O)c2ccc(C(F)(F)F)cc21] |

or a pharmaceutically acceptable salt and/or a corresponding tautomer form thereof.

In another aspect, the present invention relates to a compound of Formula (IIIA′):

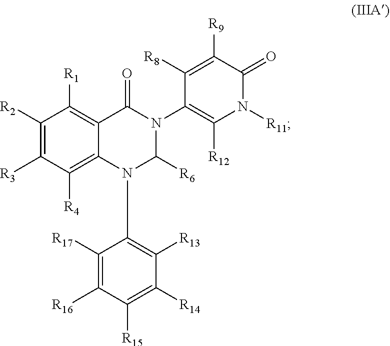

where:

R¹ or R⁴ is -hydrogen, -halogen, -straight or branched-(C₁₋₆)-alkyl or -straight or branched-(C₁₋₆)-haloalkyl;

R² or R³ is -hydrogen, -halogen, —C≡N, —OH, —NHR^{a}, —NR^{a}R^{b}, -straight or branched-(C₁₋₆)-alkyl, —(CF₂)ₙ(CH₂)ₒOH, —OR^{c} or —S(O)ₚR^{d};

where:
R¹, R², R³ or R⁴ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R⁶ is -hydrogen, -straight or branched-(C₁₋₆)-alkyl or -straight or branched-(C₁₋₆)-haloalkyl;

R⁸, R⁹ or R¹² is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;

R¹¹ is -hydrogen or -straight or branched (C₁₋₆) alkyl;

R¹³, R¹⁴, R¹⁵, R¹⁶ or R¹⁷ is -hydrogen, -halogen, —C≡N, —OR^{c}, -straight or branched (C₁₋₆) alkyl, -straight or branched (C₁₋₆)haloalkyl, —(C₃₋₆)-cycloalkyl, aryl or heteroaryl;

where:
R¹³, R¹⁴, R¹⁵, R¹⁶ or R¹⁷ optionally is substituted with hydrogen, halogen, —C≡N, —NHR^{a}, —NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;
where:
   each R^{a}, R^{b} or R^{c} of R¹, R², R³, R⁴, R⁸, R⁹, R¹¹, R¹³, R¹⁴, R¹⁵, R¹⁶ or R¹⁷ as defined above is hydrogen, -straight or branched-(C₁₋₆)-alkyl, -straight or branched-(C₁₋₆)-haloalkyl or —(C₃₋₆)-cycloalkyl;
   where:
      R^{a} optionally further substituted with —OH;
      R^{d} is hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
      where:
         each R^{a} or R^{b} of NHR^{a} or NR^{a}R^{b} as defined for R^{d} is hydrogen, -straight or branched-(C₁₋₆)-alkyl, -straight or branched-(C₁₋₆)-haloalkyl or —(C₃₋₆)-cycloalkyl;

n, o or p is 0 or an integer from 1 to 5; or

a pharmaceutically acceptable salt and/or corresponding tautomer form thereof.

In another aspect, the present invention relates to a compound which is:

| Name | Structure |
|---|---|
| 1-(4-fluoro-2-methylphenyl)-3-(2- methyl-6-oxo-1,6-dihydropyridin-3-yl)- 7-(trifluoromethyl)-2,3- dihydroquinazolin-4(1H)-one | 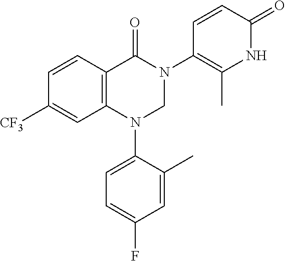 [SMILES: Cc1cc(F)ccc1N1CN(c2ccc(=O)[nH]c2C)C(=O)c2ccc(C(F)(F)F)cc21] |
| 1-(4-fluoro-2-methylphenyl)-3-(6-oxo- 1,6-dihydropyridin-3-yl)-7- (trifluoromethyl)-2,3-dihydroquinazolin- 4(1H)-one | 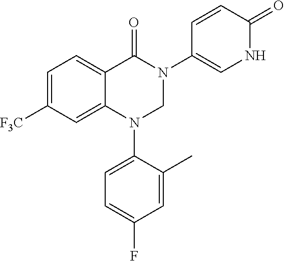 [SMILES: Cc1cc(F)ccc1N1CN(c2ccc(=O)[nH]c2)C(=O)c2ccc(C(F)(F)F)cc21] |
| 1-(4-fluoro-2,6-dimethylphenyl)-3-(6- oxo-1,6-dihydropyridin-3-yl)-7- (trifluoromethyl)-2,3-dihydroquinazolin- 4(1H)-one | 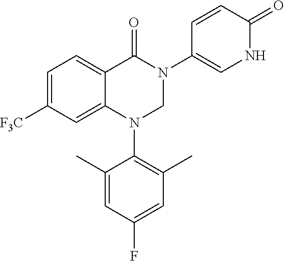 [SMILES: Cc1cc(F)cc(C)c1N1CN(c2ccc(=O)[nH]c2)C(=O)c2ccc(C(F)(F)F)cc21] |
| 1-(4-fluoro-2-methylphenyl)-2-methyl- 3-(6-oxo-1,6-dihydropyridin-3-yl)-7- (trifluoromethyl)-2,3-dihydroquinazolin- 4(1H)-one | 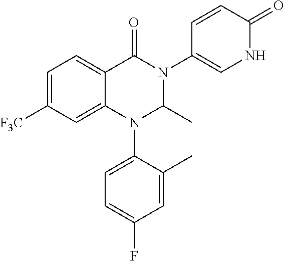 [SMILES: Cc1cc(F)ccc1N1c2cc(C(F)(F)F)ccc2C(=O)N(c2ccc(=O)[nH]c2)C1C] |
| 1-(2-chloro-4-fluorophenyl)-3-(6-oxo- 1,6-dihydropyridin-3-yl)-7- (trifluoromethyl)-2,3-dihydroquinazolin- 4(1H)-one | 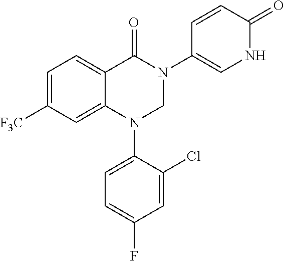 [SMILES: O=C1c2ccc(C(F)(F)F)cc2N(c2ccc(F)cc2Cl)CN1c1ccc(=O)[nH]c1] |
| 1-(4-fluoro-2-(2-hydroxyethoxy) phenyl)-3-(6-oxo-1,6-dihydropyridin-3- yl)-7-(trifluoromethyl)-2,3- dihydroquinazolin-4(1H)-one | 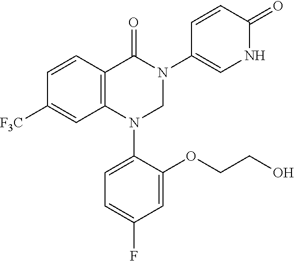 [SMILES: O=C1c2ccc(C(F)(F)F)cc2N(c2ccc(F)cc2OCCO)CN1c1ccc(=O)[nH]c1] |
| 1-(2,4-difluorophenyl)-3-(6-oxo-1,6- dihydropyridin-3-yl)-7-(trifluoromethyl)- 2,3-dihydroquinazolin-4(1H)-one | 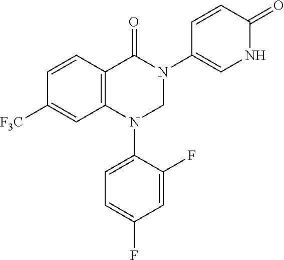 [SMILES: O=C1c2ccc(C(F)(F)F)cc2N(c2ccc(F)cc2F)CN1c1ccc(=O)[nH]c1] |
| 1-(2-ethyl-4-fluorophenyl)-3-(6-oxo-1,6- dihydropyridin-3-yl)-7-(trifluoromethyl)- 2,3-dihydroquinazolin-4(1H)-one | 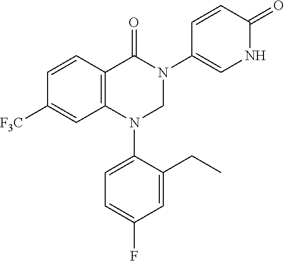 [SMILES: CCc1cc(F)ccc1N1CN(c2ccc(=O)[nH]c2)C(=O)c2ccc(C(F)(F)F)cc21] |
| 8-chloro-1-(4-fluoro-2-methylphenyl)- 3-(6-oxo-1,6-dihydropyridin-3-yl)-7- (trifluoromethyl)-2,3-dihydro quinazolin-4(1H)-one | 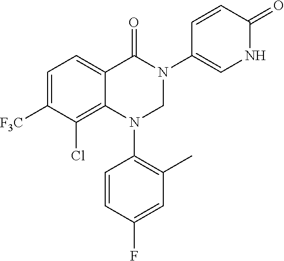 [SMILES: Cc1cc(F)ccc1N1CN(c2ccc(=O)[nH]c2)C(=O)c2ccc(C(F)(F)F)c(Cl)c21] |
| 1-(4-fluoro-2-methylphenyl)-8-methyl- 3-(6-oxo-1,6-dihydropyridin-3-yl)-7- (trifluoromethyl)-2,3-dihydroquinazolin- 4(1H)-one | 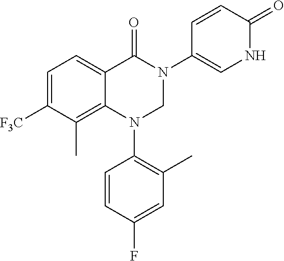 [SMILES: Cc1cc(F)ccc1N1CN(c2ccc(=O)[nH]c2)C(=O)c2ccc(C(F)(F)F)c(C)c21] |
| 1-(4-fluoro-2-isopropylphenyl)-3-(2- methyl-6-oxo-1,6-dihydropyridin-3-yl)- 7-(trifluoromethyl)-2,3- dihydroquinazolin-4(1H)-one | 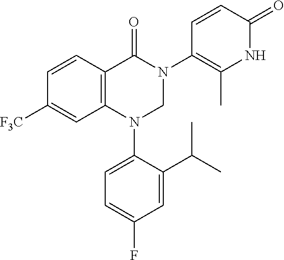 [SMILES: Cc1[nH]c(=O)ccc1N1CN(c2ccc(F)cc2C(C)C)c2cc(C(F)(F)F)ccc2C1=O] |
| 6-chloro-1-(4-fluoro-2-methylphenyl)- 3-(2-methyl-6-oxo-1,6-dihydropyridin-3- yl)-2,3-dihydroquinazolin-4(1H)-one | 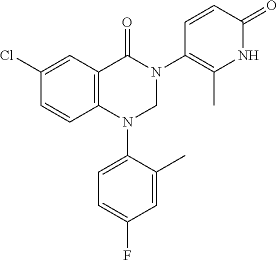 [SMILES: Cc1cc(F)ccc1N1CN(c2ccc(=O)[nH]c2C)C(=O)c2cc(Cl)ccc21] |
| 1-(4-fluorophenyl)-3-(2-methyl-6-oxo- 1,6-dihydropyridin-3-yl)-2,3- dihydroquinazolin-4(1H)-one | 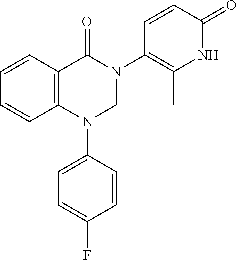 [SMILES: Cc1[nH]c(=O)ccc1N1CN(c2ccc(F)cc2)c2ccccc2C1=O] |
| 3-(2-methyl-6-oxo-1,6-dihydropyridin- 3-yl)-1-(o-tolyl)-2,3-dihydroquinazolin- 4(1H)-one | 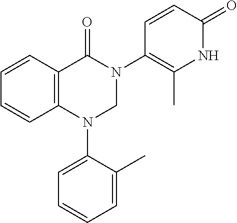 [SMILES: Cc1ccccc1N1CN(c2ccc(=O)[nH]c2C)C(=O)c2ccccc21] |
| 1-(4-fluoro-2-methylphenyl)-6-methyl- 3-(2-methyl-6-oxo-1,6-dihydropyridin-3- yl)-2,3-dihydroquinazolin-4(1H)-one | 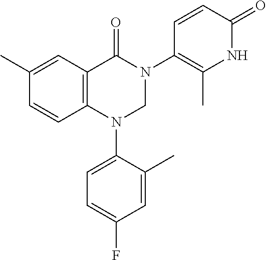 [SMILES: Cc1ccc2c(c1)C(=O)N(c1ccc(=O)[nH]c1C)CN2c1ccc(F)cc1C] |
| 1-(4-fluoro-2-methylphenyl)-3-(2- methyl-6-oxo-1,6-dihydropyridin-3-yl)- 2,3-dihydroquinazolin-4(1H)-one | 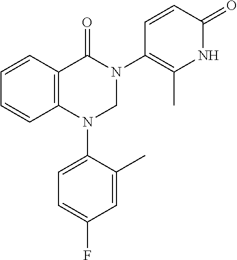 [SMILES: Cc1cc(F)ccc1N1CN(c2ccc(=O)[nH]c2C)C(=O)c2ccccc21] |
| 1-(4-fluorophenyl)-3-(6-oxo-1,6- dihydropyridin-3-yl)-7-(trifluoromethyl)- 2,3-dihydroquinazolin-4(1H)-one | 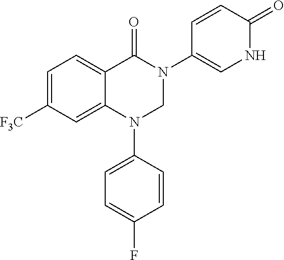 [SMILES: O=C1c2ccc(C(F)(F)F)cc2N(c2ccc(F)cc2)CN1c1ccc(=O)[nH]c1] |
| 1-(4-bromo-2-methylphenyl)-3-(6-oxo- 1,6-dihydropyridin-3-yl)-7- (trifluoromethyl)-2,3-dihydroquinazolin- 4(1H)-one | 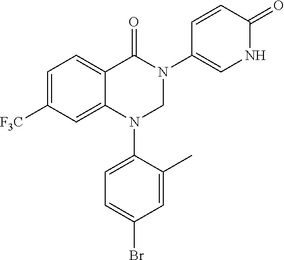 [SMILES: Cc1cc(Br)ccc1N1CN(c2ccc(=O)[nH]c2)C(=O)c2ccc(C(F)(F)F)cc21] |
| 1-(4-fluorophenyl)-3-(2-methyl-6-oxo- 1,6-dihydropyridin-3-yl)-7- (trifluoromethyl)-2,3-dihydroquinazolin- 4(1H)-one | 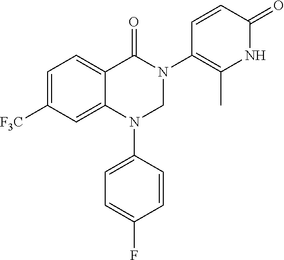 [SMILES: Cc1[nH]c(=O)ccc1N1CN(c2ccc(F)cc2)c2cc(C(F)(F)F)ccc2C1=O] |
| 3-methyl-4-(3-(2-methyl-6-oxo-1,6- dihydropyridin-3-yl)-4-oxo-7- (trifluoromethyl)-3,4-dihydroquinazolin- 1(2H)-yl)benzonitrile | 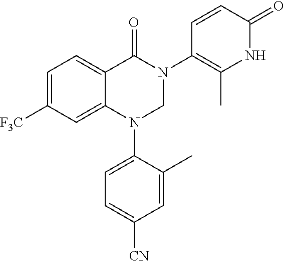 [SMILES: Cc1cc(C#N)ccc1N1CN(c2ccc(=O)[nH]c2C)C(=O)c2ccc(C(F)(F)F)cc21] |
| 3-(2-ethyl-6-oxo-1,6-dihydropyridin-3- yl)-1-(4-fluoro-2-methylphenyl)-7- (trifluoromethyl)-2,3-dihydroquinazolin- 4(1H)-one | 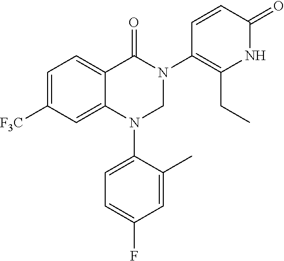 [SMILES: CCc1[nH]c(=O)ccc1N1CN(c2ccc(F)cc2C)c2cc(C(F)(F)F)ccc2C1=O] |
| 3-(2-chloro-6-oxo-1,6-dihydropyridin- 3-yl)-1-(4-fluoro-2-methylphenyl)-7- (trifluoromethyl)-2,3-dihydroquinazolin- 4(1H)-one | 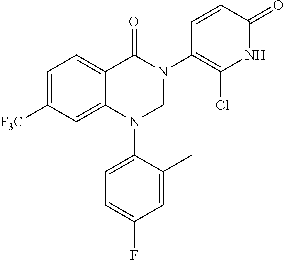 [SMILES: Cc1cc(F)ccc1N1CN(c2ccc(=O)[nH]c2Cl)C(=O)c2ccc(C(F)(F)F)cc21] |
| 7-bromo-1-(4-fluoro-2-methylphenyl)- 3-(2-methyl-6-oxo-1,6-dihydropyridin-3- yl)-2,3-dihydroquinazolin-4(1H)-one | 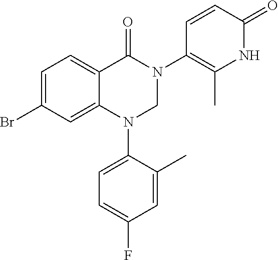 [SMILES: Cc1cc(F)ccc1N1CN(c2ccc(=O)[nH]c2C)C(=O)c2ccc(Br)cc21] |
| 1-(4-fluoro-2-methylphenyl)-3-(2- methyl-6-oxo-1,6-dihydropyridin-3-yl)- 4-oxo-1,2,3,4-tetrahydroquinazoline-7- carbonitrile | 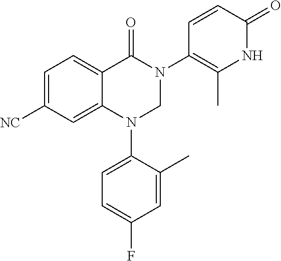 [SMILES: Cc1cc(F)ccc1N1CN(c2ccc(=O)[nH]c2C)C(=O)c2ccc(C#N)cc21] |
| 3-(1-(4-fluoro-2-methylphenyl)-4-oxo-7- (trifluoromethyl)-1,4-dihydroquinazolin- 3(2H)-yl)-6-oxo-1,6-dihydropyridine-2- carbonitrile | 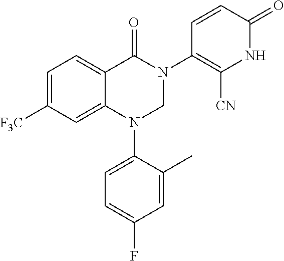 [SMILES: Cc1cc(F)ccc1N1CN(c2ccc(=O)[nH]c2C#N)C(=O)c2ccc(C(F)(F)F)cc21] |
| 1-(2,4-difluorophenyl)-3-(2-methyl-6- oxo-1,6-dihydropyridin-3-yl)-7- (trifluoromethyl)-2,3-dihydroquinazolin- 4(1H)-one | 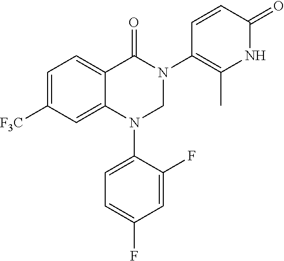 [SMILES: Cc1[nH]c(=O)ccc1N1CN(c2ccc(F)cc2F)c2cc(C(F)(F)F)ccc2C1=O] |
| 1-(4-ethoxyphenyl)-3-(2-methyl-6-oxo- 1,6-dihydropyridin-3-yl)-7- (trifluoromethyl)-2,3-dihydroquinazolin- 4(1H)-one | 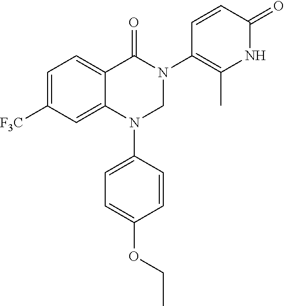 [SMILES: CCOc1ccc(N2CN(c3ccc(=O)[nH]c3C)C(=O)c3ccc(C(F)(F)F)cc32)cc1] |
| 1-(4-fluoro-2-methylphenyl)-3-(6-oxo- 1,6-dihydropyridin-3-yl)-6- (trifluoromethyl)-2,3-dihydroquinazolin- 4(1H)-one | 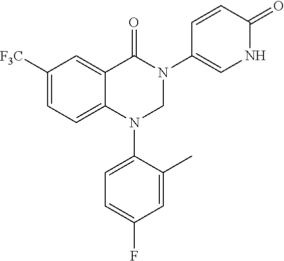 [SMILES: Cc1cc(F)ccc1N1CN(c2ccc(=O)[nH]c2)C(=O)c2cc(C(F)(F)F)ccc21] |
| 1-(4-fluoro-2-methoxyphenyl)-3-(6-oxo- 1,6-dihydropyridin-3-yl)-7- (trifluoromethyl)-2,3-dihydroquinazolin- 4(1H)-one | 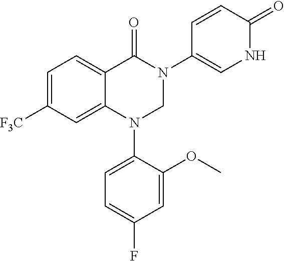 [SMILES: COc1cc(F)ccc1N1CN(c2ccc(=O)[nH]c2)C(=O)c2ccc(C(F)(F)F)cc21] |
| 1-(4-fluoro-2-methylphenyl)-3-(6-oxo- 1,6-dihydropyridin-3-yl)-5- (trifluoromethyl)-2,3-dihydroquinazolin- 4(1H)-one | 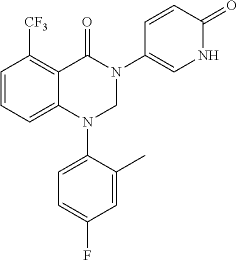 [SMILES: Cc1cc(F)ccc1N1CN(c2ccc(=O)[nH]c2)C(=O)c2c1cccc2C(F)(F)F] |
| 1-(4-fluoro-2-methylphenyl)-3-(5- methyl-6-oxo-1,6-dihydropyridin-3-yl)- 7-(trifluoromethyl)-2,3- dihydroquinazolin-4(1H)-one | 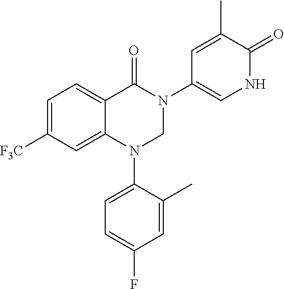 [SMILES: Cc1cc(F)ccc1N1CN(c2c[nH]c(=O)c(C)c2)C(=O)c2ccc(C(F)(F)F)cc21] |
| 1-(4-fluoro-2-methylphenyl)-3-(4- methyl-6-oxo-1,6-dihydropyridin-3-yl)- 7-(trifluoromethyl)-2,3- dihydroquinazolin-4(1H)-one | 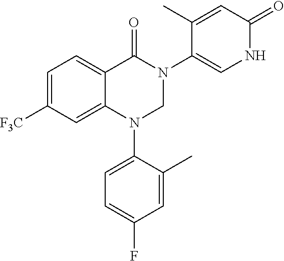 [SMILES: Cc1cc(=O)[nH]cc1N1CN(c2ccc(F)cc2C)c2cc(C(F)(F)F)ccc2C1=O] |
| 1-(4-fluoro-2-methylphenyl)-4-oxo-3-(6- oxo-1,6-dihydropyridin-3-yl)-1,2,3,4- tetrahydroquinazoline-7-carbonitrile | 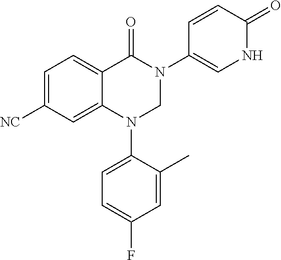 [SMILES: Cc1cc(F)ccc1N1CN(c2ccc(=O)[nH]c2)C(=O)c2ccc(C#N)cc21] |
| 1-(4-fluoro-2-methoxyphenyl)-3-(6-oxo- 1,6-dihydropyridin-3-yl)-5- (trifluoromethyl)-2,3-dihydroquinazolin- 4(1H)-one | 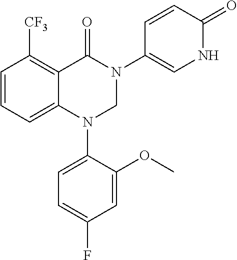 [SMILES: COc1cc(F)ccc1N1CN(c2ccc(=O)[nH]c2)C(=O)c2c1cccc2C(F)(F)F] |
| 1-(4-fluoro-2-methylphenyl)-3-(1- methyl-6-oxo-1,6-dihydropyridin-3-yl)- 7-(trifluoromethyl)-2,3- dihydroquinazolin-4(1H)-one | 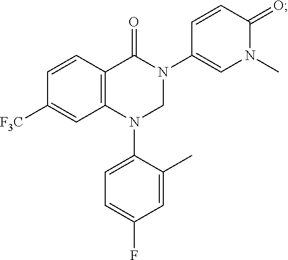 [SMILES: Cc1cc(F)ccc1N1CN(c2ccc(=O)n(C)c2)C(=O)c2ccc(C(F)(F)F)cc21] |

or a pharmaceutically acceptable salt and/or a corresponding tautomer form thereof.

In another aspect, the present invention relates to a compound which is 1-cyclohexyl-3-(6-oxo-1,6-dihydropyridin-3-yl)-7-(trifluoromethyl)-2,3-dihydroquinazolin-4(1H)-one:

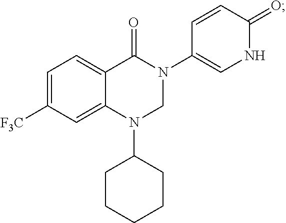 [SMILES: O=C1c2ccc(C(F)(F)F)cc2N(C2CCCCC2)CN1c1ccc(=O)[nH]c1]

a pharmaceutically acceptable salt and/or a corresponding tautomer form thereof.

In another aspect, the present invention relates to a compound of Formula (IIIA″)

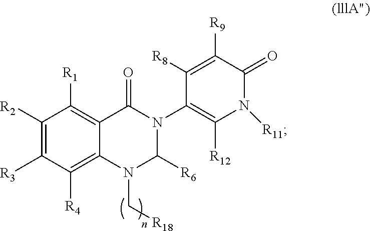

where:

R¹ or R⁴ is -hydrogen, -halogen, -straight or branched-(C₁₋₆)-alkyl or -straight or branched-(C₁₋₆)-haloalkyl;

R² or R³ is -hydrogen, -halogen, —C≡N, —OH, —NHR^{a}, —NR^{a}R^{b}, -straight or branched-(C₁₋₆)-alkyl, —(CF₂)ₙ(CH₂)ₒOH, —OR^{c} or —S(O)ₚR^{d};

where:
R¹, R², R³ or R⁴ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R⁶ is -hydrogen, -straight or branched-(C₁₋₆)-alkyl or -straight or branched-(C₁₋₆)-haloalkyl;

R¹⁸ is -2-pyridinyl, -3-pyridinyl, -4-pyridinyl, -5-pyridinyl or -6-pyridinyl;

where:
R¹⁸ optionally is substituted with hydrogen, halogen, —C≡N, —NHR^{a}, —NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;

R⁸, R⁹ or R¹² is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;

R¹¹ is -hydrogen or -straight or branched (C₁₋₆) alkyl;

where:
each R^{a}, R^{b} or R^{c} of R¹, R², R³, R⁴, R⁸, R⁹, R¹¹, R¹² or R¹⁸ as defined above is hydrogen, -straight or branched-(C₁₋₆)-alkyl, -straight or branched-(C₁₋₆)-haloalkyl or —(C₃₋₆)-cycloalkyl;
   where:
      R^{a} optionally further substituted with —OH;
      R^{d} is hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
      where:
         each R^{a} or R^{b} of NHR^{a} or NR^{a}R^{b} as defined for R^{d} is hydrogen, -straight or branched-(C₁₋₆)-alkyl, -straight or branched-(C₁₋₆)-haloalkyl or —(C₃₋₆)-cycloalkyl;

n, o or p is 0 or an integer from 1 to 5; or

a pharmaceutically acceptable salt and/or a corresponding tautomer form thereof.

In another aspect, the present invention relates to a compound which is: 3-(2-methyl-6-oxo-1,6-dihydropyridin-3-yl)-1-(2-methylpyridin-3-yl)-7-(trifluoromethyl)-2,3-dihydroquinazolin-4(1H)-one

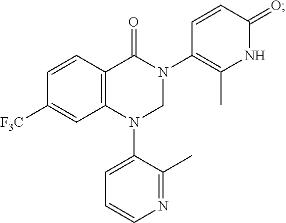 [SMILES: Cc1ncccc1N1CN(c2ccc(=O)[nH]c2C)C(=O)c2ccc(C(F)(F)F)cc21]

or
a pharmaceutically acceptable salt and/or a corresponding tautomer form thereof.

In another aspect, the present invention relates to a compound of Formula (IIIB):

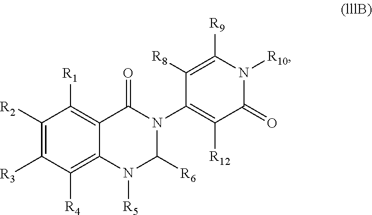

where:

R¹ or R⁴ is -hydrogen, -halogen, -straight or branched-(C₁₋₆)-alkyl or -straight or branched-(C₁₋₆)-haloalkyl;

R² or R³ is -hydrogen, -halogen, —C≡N, —OH, —NHR^{a}, —NR^{a}R^{b}, -straight or branched-(C₁₋₆)-alkyl, —(CF₂)ₙ(CH₂)ₒOH, —OR^{c} or —S(O)ₚR^{d};

where:
R¹, R², R³ or R⁴ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R⁵ is —(CH₂)ₙ-unsubstituted cyclohexyl or —(CH₂)ₙ-substituted cyclohexyl; —(CH₂)ₙ-unsubstituted phenyl or —(CH₂)ₙ-substituted phenyl; —(CH₂)ₙ-unsubstituted pyridinyl or —(CH₂)ₙ-substituted pyridinyl;

where:
R⁵ optionally is further substituted with hydrogen, halogen, —C≡N, NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;

R⁶ is -hydrogen, -straight or branched-(C₁₋₆)-alkyl or -straight or branched-(C₁₋₆)-haloalkyl;

R⁸, R⁹ or R¹² is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;

R¹⁰ is -hydrogen, -straight or branched (C₁₋₆) alkyl;

where:
each R^{a}, R^{b} or R^{c} of R¹, R², R³, R⁴, R⁸, R⁹, R¹⁰ or R¹² above is hydrogen, -straight or branched-(C₁₋₆)-alkyl, -straight or branched-(C₁₋₆)-haloalkyl or —(C₃₋₆)-cycloalkyl;
where:
   R^{a} optionally further is substituted with —OH;
   R^{d} is -hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
   where:
      each R^{a} or R^{b} of NHR^{a} or NR^{a}R^{b} as defined in R^{d} respectively, is -hydrogen, -straight or branched-(C₁₋₆)-alkyl, -straight or branched-(C₁₋₆)-haloalkyl or —(C₃₋₆)-cycloalkyl;

n, o or p is 0 or an integer from 1 to 5; or

a pharmaceutically acceptable salt and/or a corresponding tautomer form thereof.

In another aspect, the present invention relates to a compound of Formula (IIIB′):

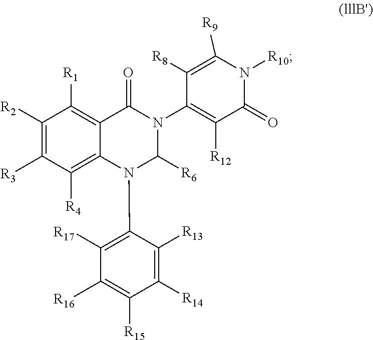

where:

R¹ or R⁴ is -hydrogen, -halogen, -straight or branched-(C₁₋₆)-alkyl or -straight or branched-(C₁₋₆)-haloalkyl;

R² or R³ is -hydrogen, -halogen, —C≡N, —OH, —NHR^{a}, —NR^{a}R^{b}, -straight or branched-(C₁₋₆)-alkyl, —(CF₂)ₙ(CH₂)ₒOH, —OR^{c} or —S(O)ₚR^{d};

where:
R¹, R², R³ or R⁴ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R⁶ is -hydrogen, -straight or branched-(C₁₋₆)-alkyl or -straight or branched-(C₁₋₆)-haloalkyl;

R⁸, R⁹ or R¹² is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;

R¹⁰ is -hydrogen or -straight or branched (C₁₋₆) alkyl;

R¹³, R¹⁴, R¹⁵, R¹⁶ or R¹⁷ is -hydrogen, -halogen, —C≡N, —OR^{c}, -straight or branched (C₁₋₆) alkyl, -straight or branched (C₁₋₆)haloalkyl, —(C₃₋₆)-cycloalkyl, aryl or heteroaryl;

where:
R¹³, R¹⁴, R¹⁵, R¹⁶ or R¹⁷ optionally is substituted with hydrogen, halogen, —C≡N, —NHR^{a}, —NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;

where:
each R^{a}, R^{b} or R^{c} of R¹, R², R³, R⁴, R⁸, R⁹, R¹⁰, R¹², R¹³, R¹⁴, R¹⁵, R¹⁶ or R¹⁷ as defined above is hydrogen, -straight or branched-(C₁₋₆)-alkyl, -straight or branched-(C₁₋₆)-haloalkyl or —(C₃₋₆)-cycloalkyl;
where:
   R^{a} optionally further substituted with —OH;
   R^{d} is hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
   where:
      each R^{a} or R^{b} of NHR^{a} or NR^{a}R^{b} as defined for R^{d} is hydrogen, -straight or branched-(C₁₋₆)-alkyl, -straight or branched-(C₁₋₆)-haloalkyl or —(C₃₋₆)-cycloalkyl;

n, o or p is 0 or an integer from 1 to 5; or

a pharmaceutically acceptable salt and/or a corresponding tautomer form thereof.

In another aspect, the present invention relates to a compound which is:

| Name | Structure |
|---|---|
| 1-(4-fluoro-2-methylphenyl)-3-(6- methyl-2-oxo-1,2-dihydropyridin-4-yl)- 7-(trifluoromethyl)-2,3- dihydroquinazolin-4(1H)-one | 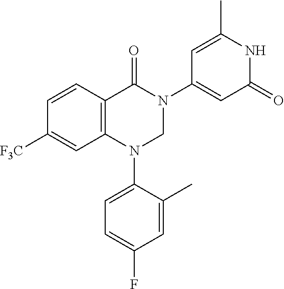 [SMILES: Cc1cc(N2CN(c3ccc(F)cc3C)c3cc(C(F)(F)F)ccc3C2=O)cc(=O)[nH]1] |
| 1-(4-fluoro-2-methylphenyl)-3-(5- methyl-2-oxo-1,2-dihydropyridin-4-yl)- 7-(trifluoromethyl)-2,3- dihydroquinazolin-4(1H)-one | 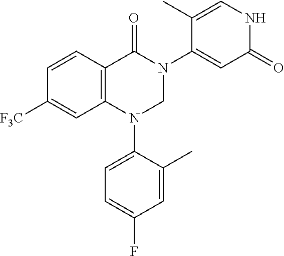 [SMILES: Cc1c[nH]c(=O)cc1N1CN(c2ccc(F)cc2C)c2cc(C(F)(F)F)ccc2C1=O] |
| 1-(4-fluoro-2-methylphenyl)-3-(3- methyl-2-oxo-1,2-dihydropyridin-4-yl)- 7-(trifluoromethyl)-2,3- dihydroquinazolin-4(1H)-one | 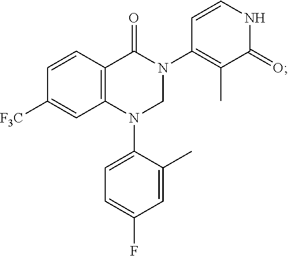 [SMILES: Cc1cc(F)ccc1N1CN(c2cc[nH]c(=O)c2C)C(=O)c2ccc(C(F)(F)F)cc21] |

or a pharmaceutically acceptable salt and/or a corresponding tautomer form thereof.

In another aspect, the present invention relates to a compound of Formula (IIIC):

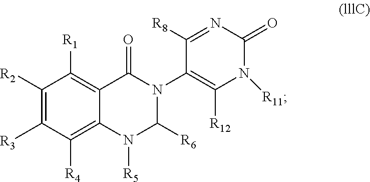

where:

R¹ or R⁴ is -hydrogen, -halogen, -straight or branched-(C₁₋₆)-alkyl or -straight or branched-(C₁₋₆)-haloalkyl;

R² or R³ is -hydrogen, -halogen, —C≡N, —OH, —NHR^{a}, —NR^{a}R^{b}, -straight or branched-(C₁₋₆)-alkyl, —(CF₂)ₙ(CH₂)ₒOH, —OR^{c} or —S(O)ₚR^{d};

where:
R¹, R², R³ or R⁴ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R⁵ is —(CH₂)ₙ-unsubstituted cyclohexyl or —(CH₂)ₙ-substituted cyclohexyl; —(CH₂)ₙ-unsubstituted phenyl or —(CH₂)ₙ-substituted phenyl; —(CH₂)ₙ-unsubstituted pyridinyl or —(CH₂)ₙ-substituted pyridinyl;

where:
R⁵ optionally is further substituted with hydrogen, halogen, —C≡N, NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;

R⁶ is -hydrogen, -straight or branched-(C₁₋₆)-alkyl or -straight or branched-(C₁₋₆)-haloalkyl;

R⁸ or R¹² is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;

R¹¹ is -hydrogen or -straight or branched (C₁₋₆) alkyl;
where:
   each R^{a}, R^{b} or R^{c} of R¹, R², R³, R⁴, R⁵, R⁶, R⁸, R¹¹ or R¹² as defined above is hydrogen, -straight or branched-(C₁₋₆)-alkyl, -straight or branched-(C₁₋₆)-haloalkyl or —(C₃₋₆)-cycloalkyl;
   where:
      R^{a} optionally further substituted with —OH;
      R^{d} is hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
      where:
         each R^{a} or R^{b} of NHR^{a} or NR^{a}R^{b} as defined for R^{d} is hydrogen, -straight or branched-(C₁₋₆)-alkyl, -straight or branched-(C₁₋₆)-haloalkyl or —(C₃₋₆)-cycloalkyl;
            n, o or p is 0 or an integer from 1 to 5; or
            a pharmaceutically acceptable salt and/or a corresponding tautomer form thereof.

In another aspect, the present invention relates to a compound of Formula (IIIC′):

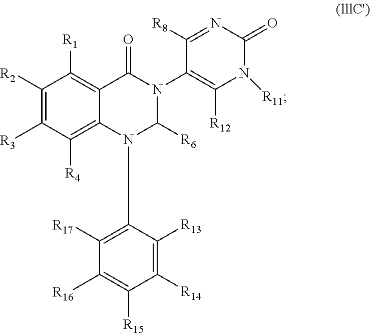

where:

R¹ or R⁴ is -hydrogen, -halogen, -straight or branched-(C₁₋₆)-alkyl or -straight or branched-(C₁₋₆)-haloalkyl;

R² or R³ is -hydrogen, -halogen, —C≡N, —OH, —NHR^{a}, —NR^{a}R^{b}, -straight or branched-(C₁₋₆)-alkyl, —(CF₂)ₙ(CH₂)ₒOH, —OR^{c} or —S(O)ₚR^{d};

where:
R¹, R², R³ or R⁴ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R⁶ is -hydrogen, -straight or branched-(C₁₋₆)-alkyl or -straight or branched-(C₁₋₆)-haloalkyl;

R⁸ or R¹² is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;

R¹¹ is -hydrogen, -straight or branched (C₁₋₆) alkyl;

R¹³, R¹⁴, R¹⁵, R¹⁶ or R¹⁷ is -hydrogen, -halogen, —C≡N, —OR^{c}, -straight or branched (C₁₋₆) alkyl, -straight or branched (C₁₋₆)haloalkyl, —(C₃₋₆)-cycloalkyl, aryl or heteroaryl;

where:
R¹³, R¹⁴, R¹⁵, R¹⁶ or R¹⁷ optionally is substituted with hydrogen, halogen, —C≡N, —NHR^{a}, —NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;
where:
   each R^{a}, R^{b} or R^{c} of R¹, R², R³, R⁴, R⁸, R¹¹, R¹², R¹³, R¹⁴, R¹⁵, R¹⁶ or R¹⁷ as defined above is hydrogen, -straight or branched-(C₁₋₆)-alkyl, -straight or branched-(C₁₋₆)-haloalkyl or —(C₃₋₆)-cycloalkyl;
   where:
      R^{a} optionally further substituted with —OH;
      R^{d} is hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
      where:
         each R^{a} or R^{b} of NHR^{a} or NR^{a}R^{b} as defined for R^{d} is hydrogen, -straight or branched-(C₁₋₆)-alkyl, -straight or branched-(C₁₋₆)-haloalkyl or —(C₃₋₆)-cycloalkyl;

n, o or p is 0 or an integer from 1 to 5; or

a pharmaceutically acceptable salt and/or a corresponding tautomer form thereof.

In another aspect, the present invention relates to a compound which is 1-(4-fluoro-2-methylphenyl)-3-(2-oxo-1,2-dihydropyrimidin-5-yl)-7-(trifluoromethyl)-2,3-dihydroquinazolin-4(1H)-one:

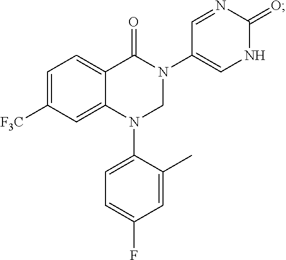 [SMILES: Cc1cc(F)ccc1N1CN(c2cnc(=O)[nH]c2)C(=O)c2ccc(C(F)(F)F)cc21]

or
a pharmaceutically acceptable salt and/or a corresponding tautomer form thereof.

In another aspect, the present invention relates to a compound of Formula (IIID):

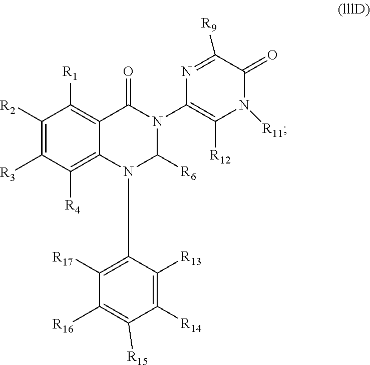

where:

R¹ or R⁴ is -hydrogen, -halogen, -straight or branched-(C₁₋₆)-alkyl or -straight or branched-(C₁₋₆)-haloalkyl;

R² or R³ is -hydrogen, -halogen, —C≡N, —OH, —NHR^{a}, —NR^{a}R^{b}, -straight or branched-(C₁₋₆)-alkyl, —(CF₂)ₙ(CH₂)ₒOH, —OR^{c} or —S(O)ₚR^{d};

where:
R¹, R², R³ or R⁴ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R⁶ is -hydrogen, -straight or branched-(C₁₋₆)-alkyl or -straight or branched-(C₁₋₆)-haloalkyl;

R⁹ or R¹² is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;

R¹¹ is -hydrogen or -straight or branched (C₁₋₆) alkyl;

R¹³, R¹⁴, R¹⁵, R¹⁶ or R¹⁷ is -hydrogen, -halogen, —C≡N, —OR^{c}, -straight or branched (C₁₋₆) alkyl, -straight or branched (C₁₋₆)haloalkyl, —(C₃₋₆)-cycloalkyl, aryl or heteroaryl;

where:
R¹³, R¹⁴, R¹⁵, R¹⁶ or R¹⁷ optionally is substituted with hydrogen, halogen, —C≡N, —NHR^{a}, —NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;
where:
   each R^{a}, R^{b} or R^{c} of R¹, R², R³, R⁴, R⁹, R¹¹, R¹², R¹³, R¹⁴, R¹⁵, R¹⁶ or R¹⁷ as defined above is hydrogen, -straight or branched-(C₁₋₆)-alkyl, -straight or branched-(C₁₋₆)-haloalkyl or —(C₃₋₆)-cycloalkyl;
   where:
      R^{a} optionally further substituted with —OH;
      R^{d} is -hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
      where:
         each R^{a} or R^{b} of NHR^{a} or NR^{a}R^{b} as defined for R^{d} is -hydrogen, -straight or branched-(C₁₋₆)-alkyl, -straight or branched-(C₁₋₆)-haloalkyl or —(C₃₋₆)-cycloalkyl;

n, o or p is 0 or an integer from 1 to 5; or

a pharmaceutically acceptable salt and/or corresponding tautomer form thereof.

In another aspect, the present invention relates to a compound which is 1-(4-fluoro-2-methylphenyl)-3-(3-methyl-5-oxo-4,5-dihydropyrazin-2-yl)-7-(trifluoromethyl)-2,3-dihydroquinazolin-4(1H)-one):

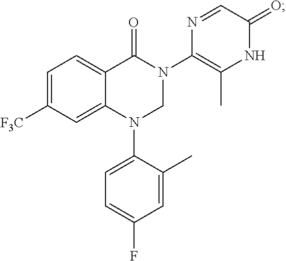 [SMILES: Cc1cc(F)ccc1N1CN(c2ncc(=O)[nH]c2C)C(=O)c2ccc(C(F)(F)F)cc21]

or
a pharmaceutically acceptable salt and/or a corresponding tautomer form thereof.

In another aspect, the present invention relates to a compound of Formula (IIIE)

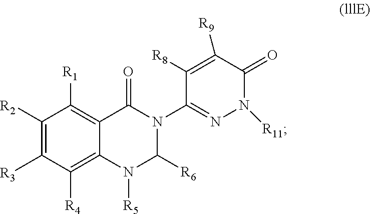

where:

R¹ or R⁴ is -hydrogen, -halogen, -straight or branched-(C₁₋₆)-alkyl or -straight or branched-(C₁₋₆)-haloalkyl;

R² or R³ is -hydrogen, -halogen, —C≡N, —OH, —NHR^{a}, —NR^{a}R^{b}, -straight or branched-(C₁₋₆)-alkyl, —(CF₂)ₙ(CH₂)ₒOH, —OR^{c} or —S(O)ₚR^{d};

where:
R¹, R², R³ or R⁴ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R⁵ is an unsaturated or saturated carbocyclic ring, unsaturated or saturated heterocyclic or heteroaryl ring;

where:
the unsaturated or saturated heterocyclic ring of R⁵ or the heteroaryl ring of R⁵, respectively, contains at least one heteroatom selected from nitrogen, oxygen or sulfur;
where:
   R⁵ optionally is substituted with hydrogen, halogen, —C≡N, —NHR^{a}, —NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;

R⁶ is -hydrogen, -straight or branched-(C₁₋₆)-alkyl or -straight or branched-(C₁₋₆)-haloalkyl;

R⁸ or R⁹ is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;

R¹¹ is -hydrogen or -straight or branched (C₁₋₆) alkyl;

where:
each R^{a}, R^{b} or R^{c} of R¹, R², R³, R⁴, R⁵, R⁸, R⁹, or R¹¹ as defined above is hydrogen, -straight or branched-(C₁₋₆)-alkyl, -straight or branched-(C₁₋₆)-haloalkyl or —(C₃₋₆)-cycloalkyl;
where:
   R^{a} optionally further substituted with —OH;
   R^{d} is hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
   where:
      each R^{a} or R^{b} of NHR^{a} or NR^{a}R^{b} as defined for R^{d} respectively, is -hydrogen, -straight or branched-(C₁₋₆)-alkyl, -straight or branched-(C₁₋₆)-haloalkyl or —(C₃₋₆)-cycloalkyl;

n, o or p is 0 or an integer from 1 to 5; or

a pharmaceutically acceptable salt and/or a corresponding tautomer form thereof.

In another aspect, the present invention relates to a compound of Formula (IIIE′):

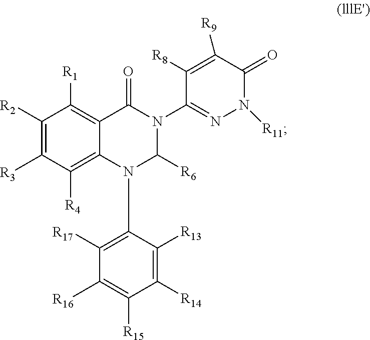

where:

R¹ or R⁴ is -hydrogen, -halogen, -straight or branched-(C₁₋₆)-alkyl or -straight or branched-(C₁₋₆)-haloalkyl;

R² or R³ is -hydrogen, -halogen, —C≡N, —OH, —NHR^{a}, —NR^{a}R^{b}, -straight or branched-(C₁₋₆)-alkyl, —(CF₂)ₙ(CH₂)ₒOH, —OR^{c} or —S(O)ₚR^{d};

where:
R¹, R², R³ or R⁴ optionally is substituted with -hydrogen, -halogen, —C≡N, —NHR^{a}, —NR^{a}R^{b}, -straight or branched-(C₁₋₆)-alkyl, -straight or branched-(C₁₋₆)-haloalkyl or —OR^{c};

R⁶ is -hydrogen, -straight or branched-(C₁₋₆)-alkyl or -straight or branched-(C₁₋₆)-haloalkyl;

R⁸ or R⁹ is -hydrogen, -halogen, —C≡N, NHR^{a}, NR^{a}R^{b}, —OR^{c}, -straight or branched (C₁₋₆) alkyl, -straight or branched (C₁₋₆)haloalkyl or —(C₃₋₆)-cycloalkyl;

R¹¹ is -hydrogen or -straight or branched (C₁₋₆) alkyl;

R¹³, R¹⁴, R¹⁵, R¹⁶ or R¹⁷ is -hydrogen, -halogen, —C≡N, —OR^{c}, -straight or branched (C₁₋₆) alkyl, -straight or branched (C₁₋₆)haloalkyl, —(C₃₋₆)-cycloalkyl, aryl or heteroaryl;

where:
R¹³, R¹⁴, R¹⁵, R¹⁶ or R¹⁷ optionally is substituted with hydrogen, halogen, —C≡N, —NHR^{a}, —NR^{a}R^{b}, —O(CH₂)ₙOH, -straight or branched-(C₁₋₆)-alkyl, -straight or branched-(C₁₋₆)-haloalkyl, —OR^{c} or —(C₃₋₆)-cycloalkyl;
where:
   each R^{a}, R^{b} or R^{c} of R¹, R², R³, R⁴, R⁸, R⁹, R¹¹, R¹³, R¹⁴, R¹⁵, R¹⁶ or R¹⁷ as defined above is hydrogen, -straight or branched-(C₁₋₆)-alkyl, -straight or branched-(C₁₋₆)-haloalkyl or —(C₃₋₆)-cycloalkyl;
   where:
      R^{a} optionally further substituted with —OH;
      R^{d} is hydrogen, —OH, NHR^{a}, NR^{a}R^{b}, -straight or branched-(C₁₋₆)-alkyl, -straight or branched-(C₁₋₆)-haloalkyl or —(C₃₋₆)-cycloalkyl;
      where:
         each R^{a} or R^{b} of NHR^{a} or NR^{a}R^{b} as defined for R^{d} is hydrogen, -straight or branched-(C₁₋₆)-alkyl, -straight or branched-(C₁₋₆)-haloalkyl or —(C₃₋₆)-cycloalkyl;

n, o or p is 0 or an integer from 1 to 5; or

a pharmaceutically acceptable salt and/or a corresponding tautomer form thereof.

In another aspect, the present invention relates to a compound which is 1-(4-fluoro-2-methylphenyl)-3-(6-oxo-1,6-dihydropyridazin-3-yl)-7-(trifluoromethyl)-2,3-dihydroquinazolin-4(1H)-one:

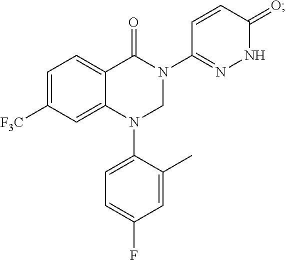 [SMILES: Cc1cc(F)ccc1N1CN(c2ccc(=O)[nH]n2)C(=O)c2ccc(C(F)(F)F)cc21]

or
a pharmaceutically acceptable salt and/or a corresponding tautomer form thereof.

It is recognized that the compounds of any of the Formulas disclosed herein, including Formula (X) and Formulas (I) to (III) (i.e., inc. corresponding subgeneric formulas defined herein), respectively, or pharmaceutically acceptable salts thereof of the present invention (i.e. as defined above and throughout the instant application) may exist in forms as stereoisomers, regioisomers, or diastereoisomers.

These compounds of the present invention may contain one or more asymmetric carbon atoms and may exist in racemic and optically active forms. For example, compounds of the present invention may exist as a racemic mixture of R(+) and S(−) enantiomers, or in separate respectively optical forms, i.e., existing separately as either the R(+) enantiomer form or in the S(+) enantiomer form. All of these individual compounds, isomers, and mixtures thereof are included within the scope of the present invention.

Moreover, compounds of the present invention may exist as tautomers or in tautomeric forms. It is conventionally understood in the chemical arts that tautomers are structural or constitutional isomers of chemical compounds that readily interconvert. This reaction commonly results in the relocation of a proton. A structural isomer, or constitutional isomer (per IUPAC^{[1]}), is a type of isomer in which molecules with the same molecular formula have different bonding patterns and atomic organization, as opposed to stereoisomers, in which molecular bonds are always in the same order and only spatial arrangement differs. The concept of tautomerizations is called tautomerism. The chemical reaction interconverting the two is called tautomerization. Care should be taken not to confuse tautomers with depictions of ‘contributing structures’ in chemical resonance. Tautomers are distinct chemical species and can be identified as such by their differing spectroscopic data, whereas resonance structures are merely convenient depictions and do not physically exist.

### Substituent Definitions

In general, the present invention relates to a compound of any of the Formulas disclosed herein, including Formula (X) and Formulas (I) to (III) (i.e., inc. corresponding subgeneric formulas defined herein), or pharmaceutically acceptable salts thereof respectively, and corresponding associated substituent or functional groups.

The definitions for the various groups and substituent groups of any of the Formulas disclosed herein, including Formula (X) and Formulas (I) to (III) (i.e., inc. corresponding subgeneric formulas defined herein) respectively, or a pharmaceutically acceptable salt and/or a corresponding tautomer form thereof (i.e., including subgeneric formulas, as defined above) provided throughout the specification are intended to particularly describe each compound species disclosed herein, individually, as well as groups of one or more compound species.

As used herein, the term alkali metal is intended to mean the Group I elements, which include, but are not limited to lithium (Li), sodium (Na), or potassium (K) and the like.

The term alkali earth metal may include, but are not limited to calcium (Ca) or magnesium (Mg) and the like.

As used herein, the terms “alkyl” or “-straight or branched (C₁₋₆) alkyl”, and the like, represents a saturated or unsaturated, straight or branched hydrocarbon moiety, which may be unsubstituted or substituted by one, or more of the substituents defined herein.

Exemplary alkyls include, but are not limited to methyl (Me), ethyl (Et), ethylene, propyl, isopropyl, butyl, butene, isobutyl, t-butyl, pentyl and the like. By way of example, the term “C₁-C₆” or “C₁₋₆” refers to an alkyl containing from 1 to 6 carbon atoms and the term “C₁-C₄” or “C₁₋₄” refers to an alkyl containing from 1 to 4 carbon atoms.

Suitably, the terms “alkyl” or “-straight or branched (C₁₋₆) alkyl” represents a saturated, straight or branched hydrocarbon moiety, which may be unsubstituted or substituted by one, or more of the substituents defined herein. Exemplary alkyls include, but are not limited to methyl (Me), ethyl (Et), propyl, isopropyl, butyl, isobutyl, t-butyl, pentyl and the like. By way of example, the term “C₁-C₆” or “C₁₋₆” refers to an alkyl containing from 1 to 6 carbon atoms and the term “C₁-C₄” or “C₁₋₄” refers to an alkyl containing from 1 to 4 carbon atoms.

When the term “alkyl” is used in combination with other substituent groups, such as “haloalkyl” or “hydroxyalkyl”, “arylalkyl”, the term “alkyl” is intended to encompass a divalent straight or branched-chain hydrocarbon radical.

The terms “halogen” and “halo” represent chloro, fluoro, bromo or iodo substituents. “Hydroxy” or “hydroxyl” is intended to mean the radical —OH.

For example, the terms “haloalkyl” or “, -straight or branched (C₁₋₆)haloalkyl” is intended to mean a saturated or unsaturated, straight or branched hydrocarbon moiety substituted with one or more halogen groups, where halogen is independently selected from: fluoro, chloro, bromo and iodo. Representative haloalkyls may include, but are not limited to trifluoromethyl (—CF₃). tetrafluoroethyl (—CF₂CHF₂), pentafluoroethyl (—CF₂CF₃) and the like.

For example, hydroxyalkyl is intended to mean a saturated or unsaturated, straight or branched hydrocarbon moiety substituted with one or more hydroxy groups.

As used herein, the term “cycloalkyl” unless otherwise defined, refers to a saturated or unsaturated non aromatic hydrocarbon ring having from three to seven carbon atoms. Cycloalkyl groups are monocyclic ring systems. For example, C₃-C₇ cycloalkyl refers to a cycloalkyl group having from 3 to 7 member atoms. Examples of cycloalkyl as used herein include: cyclopropyl, cyclobutyl, cyclopentyl, cyclohexyl, cyclobutenyl, cyclopentenyl, cyclohexenyl and cycloheptyl. Suitably cycolalkyl is selected from: cyclopropyl, cyclobutyl and cyclohexyl. Suitably “cycloalkyl” is cyclopropyl. Suitably “cyloalkyl” is cyclobutyl. Suitably “cyloalkyl” is cyclopentenyl. Suitably “cyloalkyl” is cyclohexyl.

Suitably, “cycloalkyl” refers to a non-aromatic, saturated, cyclic hydrocarbon ring. The term “—C₃₋₆ cycloalkyl” refers to a non-aromatic cyclic hydrocarbon ring having from three to six ring carbon atoms. Exemplary “—(C₃-C₆)cycloalkyl” or “—C₃₋₆ cycloalkyl” groups useful in the present invention include cyclopropyl, cyclobutyl, cyclopentyl and cyclohexyl. Suitably “cycloalkyl” is cyclopropyl. Suitably “cyloalkyl” is cyclobutyl. Suitably “cyloalkyl” is cyclopentenyl. Suitably “cyloalkyl” is cyclohexyl.

As used herein, the term “bicycloalkyl” unless otherwise defined, refers to a bridged cycloalkyl where cycloalkyl is as defined herein. Suitably the bridge is a one carbon bridge. Suitably the bridge is a two carbon bridge. Suitably the bridge is a three carbon bridge. Suitably, “bicycloalkyl” is selected from:

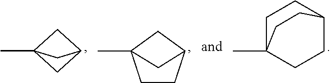

Suitably, “bicycloalkyl” is:

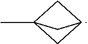

“Alkoxy” or “—OR^{c}” refers to a group containing a radical, such as a defined list of “R” alkyl substituents, attached through an oxygen linking atom. In particular, the term “—OR^{c}” is defined where the substituent variable “R^{c}” is selected from, but not limited to hydrogen, -straight or branched-(C₁₋₆)-alkyl, -straight or branched-(C₁₋₆)-haloalkyl or —(C₃₋₆)-cycloalkyl and the like. In the alternative, the term “(C₁-C₆)alkoxy” refers to a straight- or branched-chain hydrocarbon radical, having at least 1 and up to 6 carbon atoms attached through an oxygen linking atom. Exemplary “(C₁-C₄)-alkoxy” groups useful in the present invention include, but are not limited to, methoxy, ethoxy, n-propoxy, isopropoxy, n-butoxy, s-butoxy, and t-butoxy. Representative haloalkoxy may include, but are not limited to difluoromethoxy (—OCHCF₂), trifluoromethoxy (—OCF₃), tetrafluoroethoxy (—OCF₂CHF₂) and the like.

“Carbocyclic ring” refers to a ring in which all ring atoms are carbon atoms, which may be unsaturated or saturated, aromatic or non-aromatic, fused or non-fused and the like. Examples of carbocyclic rings, may include, but are not limited to cycloalkyls, such as cyclopropane, cyclobutane, cyclopentane, cyclohexane and the like, aromatic or aryl rings, which include, but are not limited to rings such as phenyl and the like.

“Carbocyclic ring” as defined above may be optionally be further substituted or defined as —CH₂-unsaturated carbocyclic ring. Examples of CH₂-unsaturated carbocyclic rings, may include, but are not limited to benzyl (i.e., —CH₂-phenyl) and the like.

“Aryl” represents an aromatic hydrocarbon ring. Aryl groups are monocyclic, bicyclic, and tricyclic ring systems having a total of five to fourteen ring member atoms, wherein at least one ring system is aromatic and wherein each ring in the system contains 3 to 7 member atoms, such as phenyl, naphthalene, and tetrahydronaphthalene. Suitably aryl is phenyl.

Suitably, “Aryl” represents a group or moiety that is an aromatic, monovalent monocyclic or bicyclic hydrocarbon radical containing at least 6 carbon ring atoms, which may be unsubstituted or substituted by one or more of the substituents defined herein, and to which may be fused one or more cycloalkyl rings, which may be unsubstituted or substituted by one or more substituents defined herein. Representative aryl groups suitable for use in the present invention, may include, but are not limited to phenyl, benzyl, and the like.

“Heteroatoms” are defined as oxygen, nitrogen, sulfur and the like. Suitably, “heteroatom” refers to a nitrogen, sulfur or oxygen atom.

“Heterocyclic” represents include heteroaryl or heterocycloalkyl groups. Heterocyclic groups may be unsaturated or saturated.

Each monocyclic heterocyclic ring of the present invention has from 3 to 7 ring atoms and contains up to four heteroatoms. Monocyclic heterocyclic rings or fused heterocyclic rings include substituted aromatic and non-aromatics.

Each fused heterocyclic ring of the present invention optionally includes carbocyclic rings or heterocyclic rings.

“Heterocycloalkyl” refers to a saturated or unsaturated non-aromatic ring containing 4 to 12 member atoms, of which 1 to 11 are carbon atoms and from 1 to 6 are heteroatoms independently selected from oxygen, nitrogen and sulfur. Heterocycloalkyl groups containing more than one heteroatom may contain different heteroatoms. Heterocycloalkyl groups are monocyclic ring systems or a monocyclic ring fused with an aryl ring or to a heteroaryl ring having from 3 to 6 member atoms. Heterocycloalkyl includes: pyrrolidinyl, tetrahydrofuranyl, dihydrofuranyl, pyranyl, tetrahydropyranyl, dihydropyranyl, tetrahydrothienyl, pyrazolidinyl, oxazolidinyl, imidazolidinyl, oxetanyl, thiazolidinyl, piperidinyl, homopiperidinyl, piperazinyl, morpholinyl, thiamorpholinyl, 1,3-dioxolanyl, 1,3-dioxanyl, 1,4-dioxanyl, 1,3-oxathiolanyl, 1,3-oxathianyl, 1,3-dithianyl, 1,3oxazolidin-2-one, hexahydro-1H-azepin, 4,5,6,7,tetrahydro-1H-benzimidazol, piperidinyl, 1,2,3,6-tetrahydro-pyridinyl and azetidinyl. Suitably, “heterocycloalkyl” includes: piperidinyl, tetrahydrofuranyl, tetrahydropyranyl, imidazolidinyl, oxetanyl, and pyrrolidinyl. Suitably, “heterocycloalkyl” is selected from: imidazolidinyl, tetrahydropyranyl and pyrrolidinyl.

Suitably, “heterocycloalkyl” is selected from: imidazolidinyl, tetrahydropyranyl, pyrrolidinyl, 1,4-dioxanyl, 1,4-oxazinyl, and oxetanyl.

Suitably, “heterocycloalkyl” represents a group or moiety comprising a non-aromatic, monovalent monocyclic or bicyclic radical, which is saturated or partially unsaturated, containing 3 to 10 ring atoms, which includes 1 to 4 heteroatoms independently selected from nitrogen, oxygen and sulfur, and which may be unsubstituted or substituted by one or more of the substituents defined herein. Generally, in the compounds of this invention, heterocycloalkyl groups are 5-membered and/or 6-membered heterocycloalkyl groups.

In one embodiment, heterocycloalkyls are formed into a pyridone ring moiety, which may include, but are not limited to: -3-pyridonyl, -4-pyridonyl, -5-pyridonyl, tetrahydropyridazin-3(2H)-one, 2,3-dihydropyrido[2,3-d] pyrimidin-4(1H)-one-yl rings or derivatives of pyridonyl substituents, such as those shown below, which may be optionally substituted:

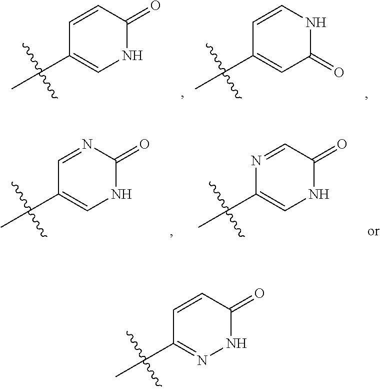 [SMILES: *c1ccc(=O)[nH]c1 ; *c1cc[nH]c(=O)c1 ; *c1cnc(=O)[nH]c1 ; *c1c[nH]c(=O)cn1 ; *c1ccc(=O)[nH]n1]

and the like.

“Heteroaryl” represents a group or moiety comprising an aromatic monovalent monocyclic or bicyclic radical, containing 4 to 10 ring atoms, suitably containing 5 to 10 ring atoms, including 1 to 4 heteroatoms independently selected from nitrogen, oxygen and sulfur, which may be unsubstituted or substituted by one or more of the substituents defined herein. This term also encompasses bicyclic heterocyclic-aryl compounds containing an aryl ring moiety fused to a heterocycloalkyl ring moiety, containing 4 to 10 ring atoms, containing 5 to 10 ring atoms, including 1 to 4 heteroatoms independently selected from nitrogen, oxygen and sulfur, which may be unsubstituted or substituted by one or more of the substituents defined herein. Heteroaryl includes but is not limited to: benzoimidazolyl, benzothiazolyl, benzothiophenyl, benzopyrazinyl, benzotriazolyl, benzotriazinyl, benzo[1,4]dioxanyl, benzofuranyl, 9H-a-carbolinyl, cinnolinyl, furanyl, pyrazolyl, imidazolyl, indolizinyl, naphthyridinyl, oxazolyl, oxothiadiazolyl, oxadiazolyl, phthalazinyl, pyridyl, pyrrolyl, purinyl, pteridinyl, phenazinyl, pyrazinyl, pyrazolopyrimidinyl, pyrazolopyridinyl, pyrrolizinyl, pyrimidyl, isothiazolyl, furazanyl, pyrimidinyl, tetrazinyl, isoxazolyl, quinoxalinyl, quinazolinyl, quinolinyl, quinolizinyl, thienyl, thiophenyl, triazolyl, triazinyl, tetrazolopyrimidinyl, triazolopyrimidinyl, tetrazolyl, thiazolyl and thiazolidinyl. Suitably heteroaryl is selected from: pyrazolyl, imidazolyl, oxazolyl and thienyl. Suitably heteroaryl is a pyridyl group or an imidazolyl group. Suitably heteroaryl is pyridyl or pyrazinyl. Suitably heteroaryl is pyridyl.

In one embodiment, heteroaryls include, but are not limited to, pyridyl (or pyridinyl), pyrazinyl, pyrimidinyl, pyridazinyl, thienyl, pyrrolyl, imidazolyl, pyrazolyl, furyl (or furanyl), isothiazolyl, furazanyl, isoxazolyl, oxazolyl, oxadiazolyl, thiazolyl, triazinyl, tetrazinyl, triazolyl, tetrazolyl and the like.

Suitably, heteroaryl is selected from: pyridyl, pyrimidinyl, pyridazinyl, pyrazolyl, furanyl, thiophenyl and thiazolyl.

Generally, the heteroaryl groups present in the compounds of this invention are 5-membered and/or 6-membered monocyclic heteroaryl groups. Selected 5-membered heteroaryl groups contain one nitrogen, oxygen or sulfur ring heteroatom, and optionally contain at least 1, 2 or 3 additional nitrogen ring atoms. Selected 6-membered heteroaryl groups contain at least 1, 2, 3 or 4 nitrogen ring heteroatoms. Selected 5- or 6-membered heteroaryl groups, may include, but not limited to pyridyl (or pyridinyl), pyrazinyl, pyrimidinyl, pyridazinyl, thienyl, pyrrolyl, imidazthienyl, pyrrolyl, imidazolyl, pyrazolyl, furyl, isothiazolyl, furazanyl, isoxazolyl, oxazolyl, oxadiazolyl, thiazolyl, triazolyl, tetrazolyl and the like.

“Oxo” represents a double-bonded oxygen moiety; for example, if attached directly to a carbon atom forms a carbonyl moiety (C═O), or attached to an N or S forms oxides, N-oxides, sulfones or sulfoxides.

As used herein, the term “compound(s) of the invention” means a compound of any of the Formulas disclosed herein, in any form, i.e., any salt or non-salt form (e.g., as a free acid or base form, or as a pharmaceutically acceptable salt thereof) and any physical form thereof (e.g., including non-solid forms (e.g., liquid or semi-solid forms), and solid forms (e.g., amorphous or crystalline forms, specific polymorphic forms, solvates, including hydrates (e.g., mono-, di- and hemi-hydrates)), and mixtures of various forms.

As used herein, the term “optionally substituted” means that a group, such as, which may include, but is not limited to alkyl, aryl, heteroaryl, etc., may be unsubstituted, or the group may be substituted with one or more substituent(s) as defined herein throughout the instant specification. In the case where groups may be selected from a number of alternative groups the selected groups may be the same or different. For example, various substituent groups of compound formulas as defined in the present invention may be optionally substituted, but are not limited to substituents, such as -hydrogen, -halogen, —C≡N, amino, substituted amino groups, alkoxy, straight or branched (C₁₋₆) alkyl, -straight or branched (C₁₋₆)haloalkyl or —(C₃₋₆)-cycloalkyl and the like.

The term “independently” means that where more than one substituent is selected from a number of possible substituents, those substituents may be the same or different.

### Enantiomers, Diastereomers and Polymorphs

The compounds according to any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) of the present invention as defined herein, may contain one or more asymmetric center(s) (i.e., also referred to as a chiral center) and may, therefore, exist as individual enantiomers, diastereomers, or other stereoisomeric forms, or as mixtures thereof.

Chiral centers, such as chiral carbon atoms, may also be present in a substituent such as an alkyl group. Where the stereochemistry of a chiral center present in any of the Formulas disclosed herein, including Formula (X) and Formulas (I) to (III) (i.e., including corresponding subgeneric formulas defined herein), respectively, or a pharmaceutically acceptable salt and/or a corresponding tautomer form thereof (i.e., including subgeneric formulas, as defined above) of the present invention, or in any chemical structure illustrated herein, is not specified the structure is intended to encompass all individual stereoisomers and all mixtures thereof. Thus, compounds or a pharmaceutically acceptable salt and/or a corresponding tautomer form thereof of the present invention, containing one or more chiral center may be used as racemic mixtures, enantiomerically enriched mixtures, or as enantiomerically pure individual stereoisomers.

Individual stereoisomers of a compound according to any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) of the present invention, which contain one or more asymmetric center may be resolved by methods known to those skilled in the art. For example, such resolution may be carried out:

(1) by formation of diastereoisomeric salts, complexes or other derivatives;

(2) by selective reaction with a stereoisomer-specific reagent, for example by enzymatic oxidation or reduction; or

(3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form.

Alternatively, specific stereoisomers may be synthesized by asymmetric synthesis using optically active reagents, substrates, catalysts or solvents, or by converting one enantiomer to the other by asymmetric transformation.

When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.”

It is to be understood that when named or depicted by structure, the disclosed compound, or solvates (particularly, hydrates) thereof, also include all polymorphs thereof.

Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

### Salts

Because of their potential use in medicine, the salts of the compounds of any of the Formulas disclosed herein, including Formula (X) and Formulas (I) to (III) (i.e., inc. corresponding subgeneric formulas defined herein), respectively (i.e., including subgeneric formulas, as defined above) of the present invention, are preferably pharmaceutically acceptable salts. Suitable pharmaceutically acceptable salts include those described by Berge, Bighley and Monkhouse J. Pharm. Sci (1977) 66, pp 1-19.

When a compound of the invention is a base (contain a basic moiety), a desired salt form may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, γ-hydroxybutyrates, glycollates, tartrates mandelates, and sulfonates, such as xylenesulfonates, methanesulfonates, propanesulfonates, naphthalene-1-sulfonates and naphthalene-2-sulfonates.

If an inventive basic compound is isolated as a salt, the corresponding free base form of that compound may be prepared by any suitable method known to the art, including treatment of the salt with an inorganic or organic base, suitably an inorganic or organic base having a higher pKₐ than the free base form of the compound.

When a compound of the invention is an acid (contains an acidic moiety), a desired salt may be prepared by any suitable method known to the art, including treatment of the free acid with an inorganic or organic base, such as an amine (primary, secondary, or tertiary), an alkali metal or alkaline earth metal hydroxide, or the like. Illustrative examples of suitable salts include organic salts derived from amino acids such as glycine and arginine, ammonia, primary, secondary, and tertiary amines, and cyclic amines, such as ethylene diamine, dicyclohexylamine, ethanolamine, piperidine, morpholine, and piperazine, as well as inorganic salts derived from sodium, calcium, potassium, magnesium, manganese, iron, copper, zinc, aluminum, and lithium.

Certain of the compounds of this invention may form salts with one or more equivalents of an acid (if the compound contains a basic moiety) or a base (if the compound contains an acidic moiety). The present invention includes within its scope all possible stoichiometric and non-stoichiometric salt forms.

Because the compounds of this invention may contain both acid and base moieties, pharmaceutically acceptable salts may be prepared by treating these compounds with an alkaline reagent or an acid reagent, respectively. Accordingly, this invention also provides for the conversion of one pharmaceutically acceptable salt of a compound of this invention, e.g., a hydrochloride salt, into another pharmaceutically acceptable salt of a compound of this invention, e.g., a sodium salt.

### Solvates

For solvates of the compounds of the invention, or pharmaceutically acceptable salts thereof, that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

### Deuterated Compounds

The invention also includes various deuterated forms of the compounds of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) of the present invention. Each available hydrogen atom attached to a carbon atom may be independently replaced with a deuterium atom.

A person of ordinary skill in the art will know how to synthesize deuterated forms of the compounds of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) of the present invention. For example, deuterated materials, such as alkyl groups may be prepared by conventional techniques (see for example: methyl-d₃-amine available from Aldrich Chemical Co., Milwaukee, Wis., Cat. No. 489,689-2).

### Isotopes

The subject invention also includes isotopically-labeled compounds which are identical to those recited in any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) of the present invention but for the fact that one or more atoms are replaced by an atom having an atomic mass or mass number different from the atomic mass or mass number most commonly found in nature.

Examples of isotopes that can be incorporated into compounds of the invention include isotopes of hydrogen, carbon, nitrogen, oxygen, fluorine, iodine and chlorine such as ³H, ¹¹C, ¹⁴C, ¹⁸F, ¹²³I or ¹²⁵I.

Compounds of the present invention and pharmaceutically acceptable salts of said compounds that contain the aforementioned isotopes and/or other isotopes of other atoms are within the scope of the present invention. Isotopically labeled compounds of the present invention, for example those into which radioactive isotopes such as ³H or ¹⁴C have been incorporated, are useful in drug and/or substrate tissue distribution assays. Tritiated, i.e. ³H, and carbon-14, ie. ¹⁴C, isotopes are particularly preferred for their ease of preparation and detectability. ¹¹C and ¹⁸F isotopes are particularly useful in PET (positron emission tomography).

### Purity

Because the compounds of the present invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

### Synthetic Schemes and General Methods of Preparation

The compounds of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) of the present invention, may be made by processes or methods of making the aforementioned compounds or pharmaceutically acceptable salts thereof obtained by using synthetic procedures illustrated in the Schemes below or by drawing on the knowledge of a skilled organic chemist.

The synthesis provided in these Schemes are applicable for producing compounds of the invention having a variety of different R¹ and R² groups employing appropriate precursors, which are suitably protected if needed, to achieve compatibility with the reactions outlined herein. Subsequent deprotection, where needed, affords compounds of the nature generally disclosed. While the Schemes are shown with compounds only of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) of the present invention, they are illustrative of processes that may be used to make the compounds of the invention.

Intermediates (compounds used in the preparation of the compounds of the invention) may also be present as salts. Thus, in reference to intermediates, the phrase “compound(s) of formula (number)” means a compound having that structural formula or a pharmaceutically acceptable salt thereof.

The present invention also relates to processes for making compounds of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) of the present invention.

The compounds of the present invention may be obtained by using synthetic procedures illustrated in Schemes below or by drawing on the knowledge of a skilled organic chemist.

The synthesis provided in these Schemes are applicable for producing compounds of the invention as defined by any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) of the present invention, respectively, having a variety of different functional groups as defined employing appropriate precursors, which are suitably protected if needed, to achieve compatibility with the reactions outlined herein. Subsequent deprotection, where needed, affords compounds of the nature generally disclosed. While the Schemes shown with compounds only as defined therein, they are illustrative of processes that may be used to make the compounds of the invention.

Intermediates (compounds used in the preparation of the compounds of the invention) also may be present as salts. Thus, in reference to intermediates, the phrase “compound(s) of formula (number)” means a compound having that structural formula or a pharmaceutically acceptable salt thereof.

The compounds according to any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) of the present invention, are prepared using conventional organic syntheses. Suitable synthetic routes are depicted below in the following general reaction schemes.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

### Synthetic Schemes

For the convenience of the reader, note the following substituent groups of compounds described in the Schemes represent, correspond and/or equivalent to substituent groups defined for compounds of any of the Formulas disclosed herein, including Formula (X) and Formulas (I) to (III) (i.e., inc. corresponding subgeneric formulas defined herein) defined in the present application:

### 1. General Methods of Preparation

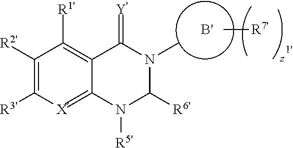

The compounds of the present invention may be obtained by using the procedures illustrated in the Schemes below, or by applying appropriate synthetic organic chemistry procedures and methodology known to those of skill in the art. The methods provided in these Schemes can be used to prepare compounds of the invention containing a variety of different X′, R^{1′}, R^{2′}, R^{3′}, R^{4′}, R^{5′}, R^{6′}, R^{7′} and B′ groups (descriptions shown above for compounds of Formulas X to XIV) employing appropriate precursors. Those skilled in the art will appreciate that in the preparation of compounds of the invention (e.g., compounds of Formula (X), tautomers thereof, salts thereof, and/or solvates thereof), it may be necessary and/or desirable to protect one or more sensitive groups in the molecule or the appropriate intermediate to prevent undesirable side reactions. Suitable protecting groups for use according to the present invention are well-known to those skilled in the art and may be used in a conventional manner. See for example, “Protective Groups in Organic Synthesis” by T. W. Green and P. G. M Wets (Wiley & Sons, 1991) or “Protecting Groups” by P. J. Kocienski (Georg Thieme Verlag, 1994). Subsequent deprotection, where needed, affords compounds of the nature generally disclosed. While the Schemes shown below are representative of methods for preparing compounds of Formula (X), they are only intended to be illustrative of processes that may be used to make the compounds of the invention.

Compound names were generated using the software naming program ChemDraw Ultra v12.0, available from Perkin Elmer, 940 Winter Street, Waltham, Mass., 02451, USA. (http://www.perkinelmer.com/).

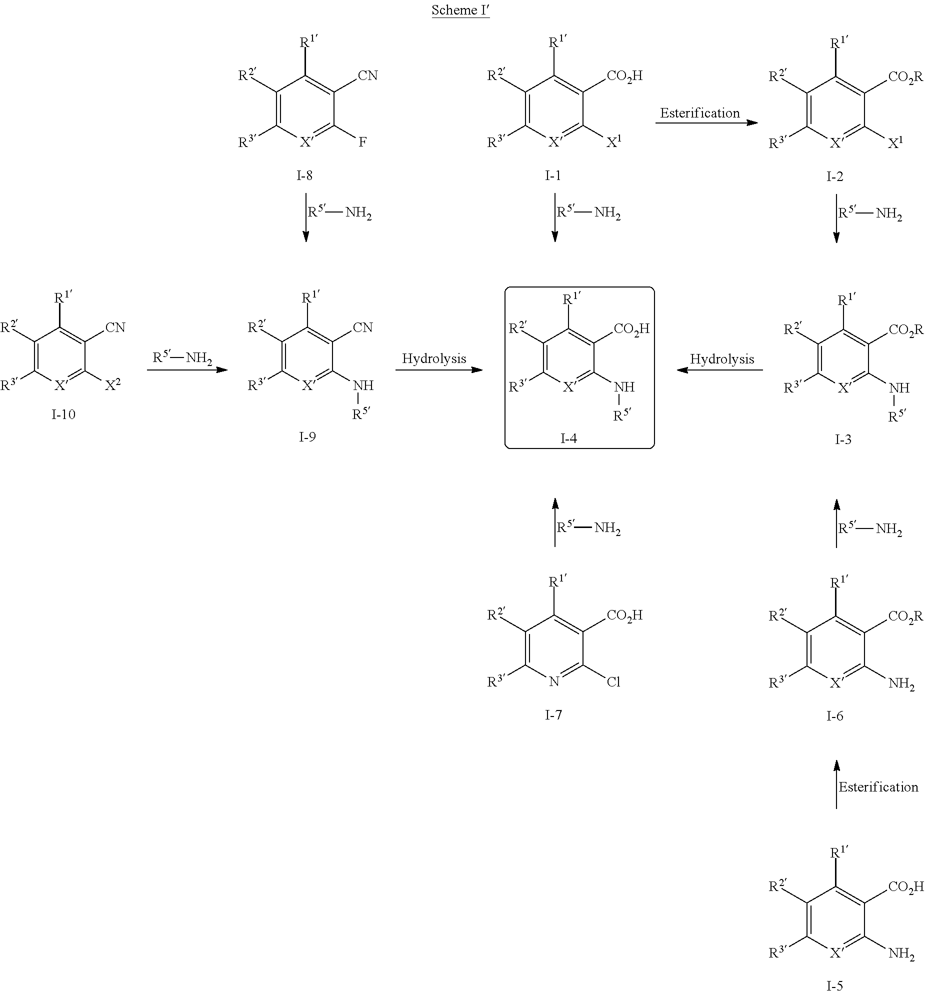

The preparation of the compounds of the present invention typically begins with the synthesis of N-substituted-2-aminoaromatic acid derivatives I-4 (Scheme I′). Esterification of a suitably substituted 2-halo aromatic acid under standard conditions could provide the corresponding ester I-2. Typically, esterification reactions can be performed under either acidic conditions, in the presence of an alcohol, or under basic conditions, in the presence of a suitable alkyl halide. Reaction of the 2-halo aromatic ester I-2 (X¹=Cl, Br or I) with an appropriate aniline or amine (R^{5′}—NH₂) provides the corresponding N-substituted-2-aminoaromatic esters I-3. Typically, this reaction can be performed at elevated temperature, using either standard heating or microwave irradiation, in the presence of a catalyst, for example Pd₂(dba)₃ or Cu/CuO, a suitable ligand, for instance BINAP or Xantphos, and an inorganic base, typically Cs₂CO₃ or K₂CO₃, in an appropriate solvent, such as 1,4-dioxane, toluene or 2-ethoxyethanol. In some cases where X¹=F, the conversion may be achieved through a S_{N}Ar reaction in the presence of a base, for example DIPEA in an appropriate solvent like DMF.

The intermediate I-3 can also be prepared from 2-aminoaromatic acid I-5 by the similar synthesis steps and reaction conditions as described above. After esterification, the resulting amino-aromatic ester I-6 can be reacted with an appropriate aryl halide (R^{5′}—X) under the similar coupling conditions and provide the corresponding I-3, where X could be Cl, Br or I. Such reactions are well-known to those of skill in the art.

Saponification of the ester I-3 to the corresponding N-substituted-2-aminoaromatic acid derivatives (I-4) is typically achieved under standard basic conditions, using bases such as LiOH, KOH, or NaOH, in a suitable solvent or solvent system, for instance methanol/H₂O, ethanol/H₂O, or THF/H₂O. Such conditions are well-known to those of skill in the art.

An alternative approach, which will be readily apparent to those of skill in the art, is to react the 2-bromoaromatic acid I-1 with an appropriate aniline or amine (R^{5′}—NH₂) to provide compound I-4 directly. The reaction conditions are similar to those described above for conversion of I-2 to I-3, but use of a ligand may, or may not, be necessary.

In some instances where X′=N, a suitably substituted 2-chloronicotinic acid I-7 can be reacted with an appropriate aniline or amine (R^{5′}—NH₂) to provide 2-aminoaromatic acid I-5 under acidic condition, such as p-toluenesulfonic acid or acetic acid, at elevated temperature with or without a base such as pyridine. The reactions are also able to be performed under basic conditions such as in presence of LiHMDS in an appropriate solvent like THF at ambient temperature.

In another alternative approach, the intermediate N-substituted-2-aminoaromatic acid derivatives I-4 can be prepared starting from suitably substituted 2-fluoro-aromatic nitriles I-8, using an S_{N}Ar reaction. For example, compound I-7 can be reacted with an appropriate aniline or amine (R^{5′}—NH₂) to effect the desired S_{N}Ar reaction to afford the substitution product I-9. This reaction is typically conducted in the presence of a base, oftentimes NaH or K₂CO₃, in an appropriately polar solvent such as DMF. This reaction can be done at either room temperature or with heating, depending on the relative reactivity of the starting materials. The nitrile group of I-9 is then hydrolyzed to the corresponding carboxylic acid I-4 by reaction with a hydroxylic base, usually LiOH, KOH, or NaOH, in a suitable solvent or solvent system, for instance methanol/H₂O, ethanol/H₂O, or THF/H₂O. I-9 can also be obtained from a suitably substituted 2-halo-aromatic nitriles I-10 (X²=Cl or Br) through a traditional cross-coupling reaction. The reaction conditions are similar to those described above for conversion of I-2 to I-3. Such reactions are well-known to those of skill in the art.

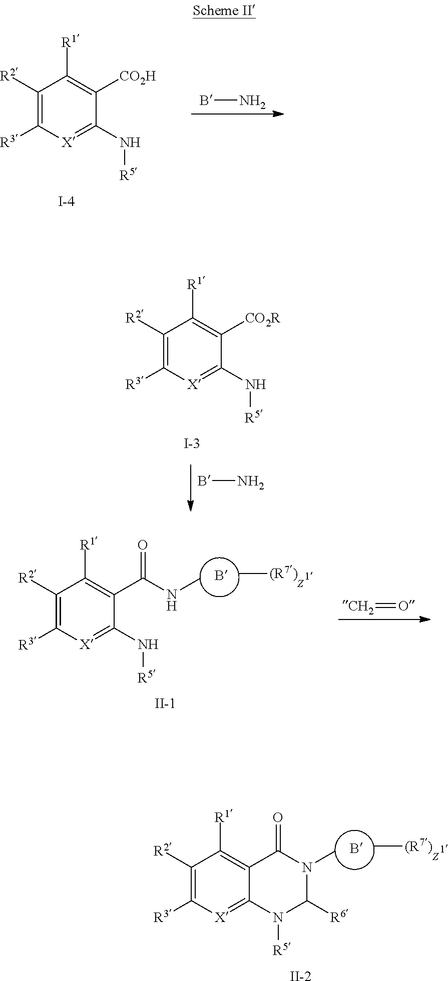

The intermediate N-substituted-2-aminoaromatic acid derivatives I-4, prepared as illustrated in Scheme I′, can be converted to II-2 as outlined in Scheme II′. Coupling of I-5 with a suitable 2-alkoxy-azaheterocycle B′—NH₂, for example 2-methoxy-4-aminopyridine, under various amide couple conditions known to those of skill in the art, provides the corresponding amide II-1. For example, one might employ standard coupling reagents, like EDC/HOBT, HATU, HBTU or T3P, in the presence of an amine base, like triethylamine, or Hünig's base (diisopropylethylamine), in a suitable solvent, typically DMF, DMA or acetonitrile. Alternatively, one might convert the acid to the corresponding acid chloride, using a reagent like thionyl chloride or oxalyl chloride, then react the acid chloride with a suitable 2-alkoxy-azaheterocycle B′—NH₂ (like 2-methoxy-4-aminopyridine), in the presence of an acid scavenger or base, such as pyridine, 2,6-lutidine, triethylamine or Hünig's base, in an appropriate solvent, such as dichloromethane or pyridine, to afford the desired coupling product II-1. Alternatively, II-1 may be formed directly from N-substituted-2-aminoaromatic esters I-3 by treating the mixture of I-3 and the corresponding B′—NH₂ with DABAL-Me₃ at elevated temperature in an appropriate solvent such as THF.

Formation of the dihydroquinazolinone ring system, as in II-2, involves reaction of II-1 with formaldehyde or a suitable equivalent. For instance, the reaction may be achieved using formaldehyde, either as gaseous formaldehyde, paraformaldehyde, or s-trioxane, in the presence of an acid, preferably PTSA or sulfuric acid. For the case of R6′ is a carbonyl oxygen, CDI and DBU could be used. The reaction could be conducted at elevated temperature, using chloroform, toluene or 2,2,2-trifluoroethanol as the solvent. Alternatively, the dihydroquinazolinone ring system can be formed via reaction of II-1 using diiodomethane or chloroiodomethane as a formaldehyde equivalent. In this variant of the cyclization reaction, a base, typically Cs₂CO₃ or NaH, could be used, in a suitable solvent, oftentimes acetonitrile or DMF. The choice of using formaldehyde or diiodomethane depends on the particular reactivity characteristics of the substrate I-1.

In some examples, compound II-2 can be obtained as the final product, which may also be accessed through the method described in Scheme III′ and Scheme IV′.

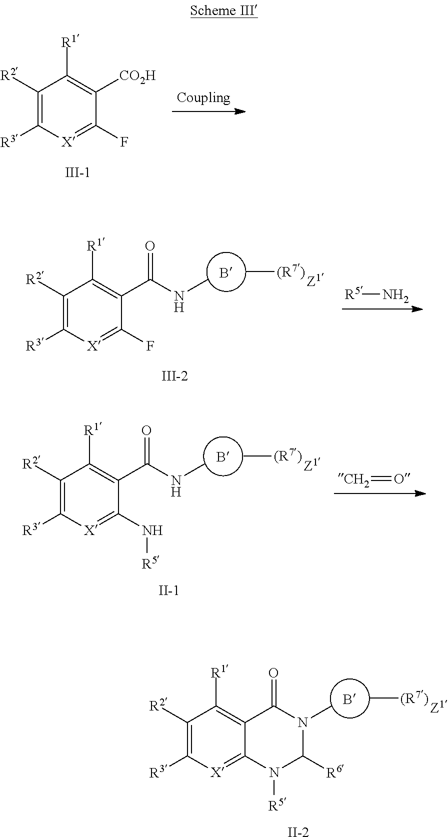

In a variation of the methods described in Schemes I′ and II′, the compounds of the present invention can be prepared as illustrated in Scheme III′. Coupling of III-1 with a suitable 2-alkoxy-azaheterocycle B′—NH₂, for example 2-methoxy-4-aminopyridine, under various amide couple conditions known to those of skill in the art, provides the corresponding amide III-2. General conditions for forming amides are described in Scheme II′. Subsequently, compound III-2 can be reacted with an appropriate aniline or amine (R^{5′}—NH₂) to effect an S_{N}Ar reaction to afford product II-1. This reaction can be conducted in the presence of a base, for instance Cs₂CO₃, LiHMDS or NaH, in a neutral solvent such as THF.

This reaction can be done at either room temperature or with heating, depending on the relative reactivity of the 2-fluoro-aromatic carboxylic acid starting material. Compound II-1 can then be converted to compound II-2 according to the methods illustrated in Scheme II′.

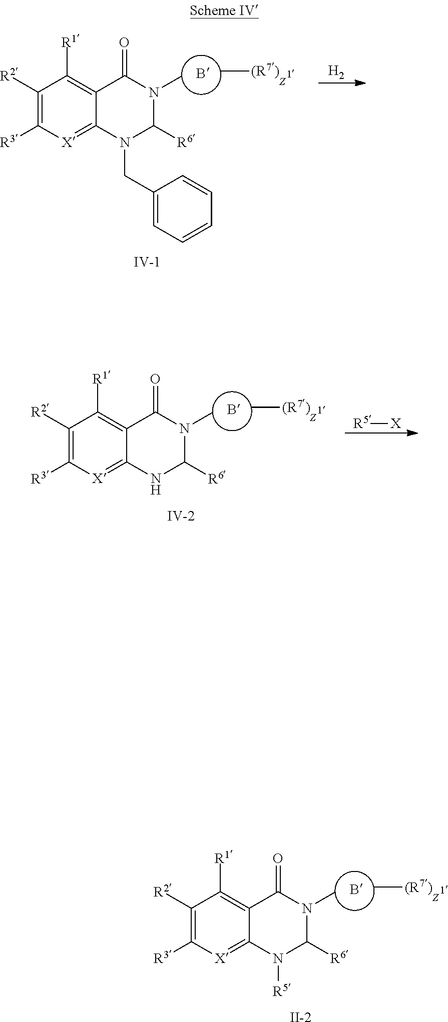

An alternative method for preparing the compounds of the present invention is shown in Scheme II′-2. Using chemistry similar to that described in Schemes I′, II′, and III′, compounds like IV-1 can be readily prepared. N-Debenzylation of IV-1 may be achieved via hydrogenation using a catalyst such as Pd/C and a hydrogen source (e.g. hydrogen gas or ammonium formate), to afford the corresponding N-debenzylated derivatives IV-2. These compounds can be N-arylated via reaction with aryl halides under appropriate conditions. For example, IV-2 can react with a suitably functionalized aryl chloride, bromide or iodide, in the presence of a catalyst, for example Pd₂(dba)₃, Cu₂O, or Cu/CuO, and a suitable ligand, for instance BINAP or Xantphos. The reaction could be conducted at elevated temperature in the presence of an inorganic base, typically NaOtBu, Cs₂CO₃ or K₃PO₄, in an appropriate solvent, such as 1,4-dioxane, toluene or DMSO. With certain aryl halides, IV-2 can be induced to participate in a nucleophilic aromatic substitution reaction to provide II-2. For example, reaction of IV-2 with certain 2-haloheterocycles, in the presence of a base like Cs₂CO₃, NaH or LiHMDS, in a neutral solvent like THF, NMP or DMF, and typically at elevated temperature, can provide the N-aryl derivatives II-2.

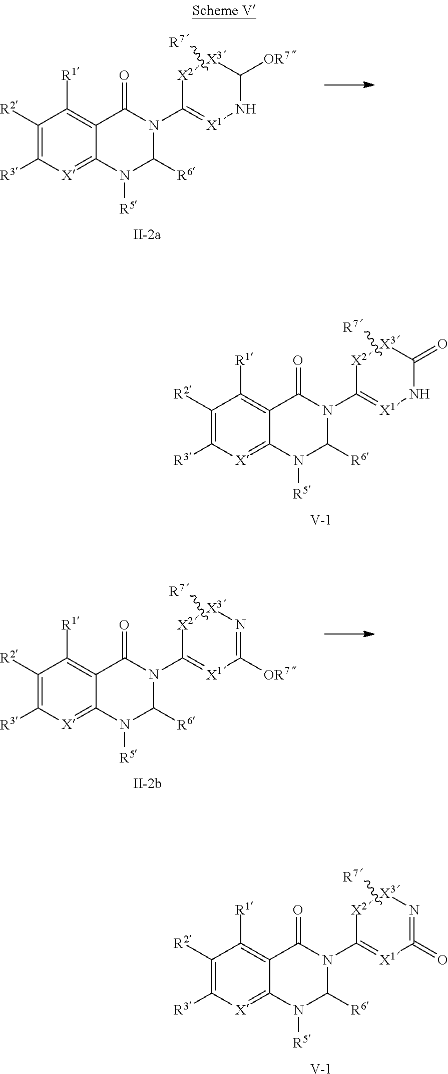

In the instance where B′=2-alkoxy-azaheterocycle, for example 2-methoxy-5-aminopyridine, 2-methoxy-4-aminopyridine or 2-methoxy-4-aminopyrimidine, etc., removal of the alkoxy (typically methoxy) protecting group may be required to complete the synthesis of the compounds of the present invention. Preferred methods for achieving this transformation include reaction with a mixture of TMS-chloride and NaI, or a solution of TMS-iodide, in a neutral solvent like acetonitrile, at elevated temperature. Alternatively, this conversion may be achieved utilizing a mixture of p-toluenesulfonic acid and LiCl in a solvent such as DMF at elevated temperature. The reaction also can be realized with pyridine hydrobromide in a solvent of pyridine at elevated temperature.

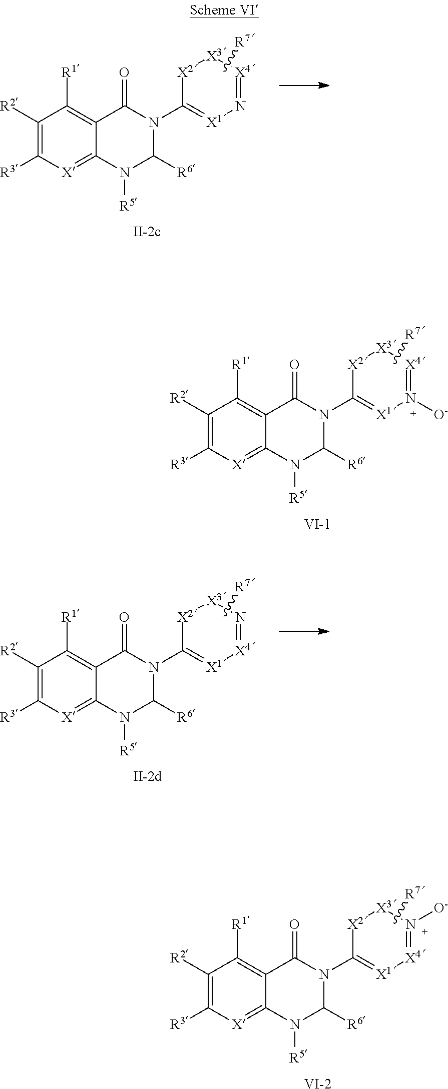

In the instance where B′=azaheterocycle such as 3-aminopyridine or 4-aminopyridine etc., an oxidation step may be required to generate the corresponding pyridine N-oxide analogs of the present invention. The conversions are usually achieved in the presence of an oxidant, for example mCPBA, in a neutral solvent (e.g. DCM) at 0° C. or room temperature.

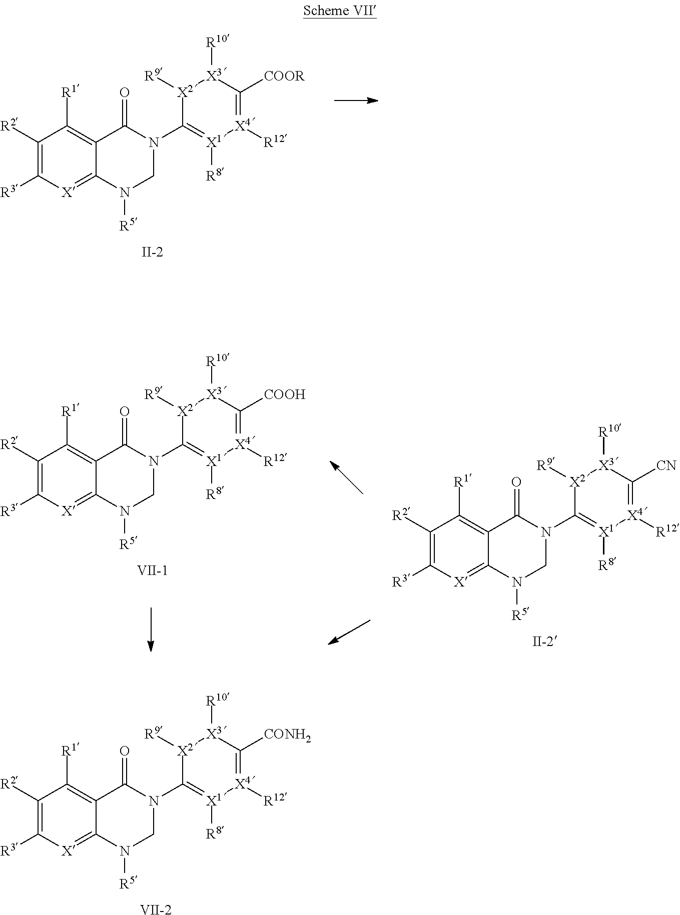

In the instance where B′ ring in the final compound is aromatic acid VII-1, it may be prepared from the corresponding aromatic ester II-2 through hydrolysis. Similar as the reaction condition for conversion of I-3 to I-4, the reaction is usually completed in the presence of an inorganic base such as LiOH, KOH, or NaOH in a suitable solvent or solvent system, for instance methanol/H2O, ethanol/H₂O, or THF/H₂O. Further react VII-1 with ammonium acetate in the presence of a coupling reagent like HATU and an amine base Hünig's base (diisopropylethylamine), in a suitable solvent, typically DMF, to provide another final compound VII-2.

VII-1 and VII-2 may also synthesized from suitably substituted aromatic nitrile II-2′ under hydrolytic conditions as described for the reaction from I-9 to I-4.

It will be obvious to those of skill in the art that the chemistry as illustrated in Scheme VII′, is representative of a general method, and that the analogs with other aromatic rings or substituents at other positions can be used.

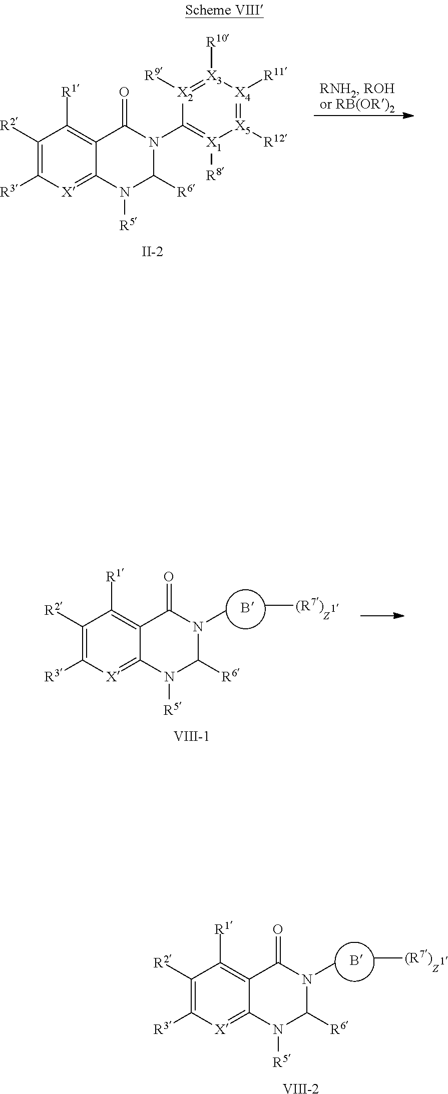

In the instance where R^{8′}, R^{9′}, R^{10′}, R^{11′} or R^{12′} in II-2′ is substituted by appropriate halogens, particularly chlorine, bromine, or iodine, the halogen can be replaced with other functionalities by reaction with a corresponding coupling partner under appropriate coupling reaction conditions. The coupling partners include suitable amine, alcohol and boronic acid or ester. This type of reaction usually can be realized at elevated temperature, using either standard heating or microwave irradiation, in the presence of a catalyst, usually Pd₂(dba)₃, a suitable ligand, for instance tBuXphos, XPhos or Xantphos, and an inorganic base, typically KOH, Cs₂CO₃ or K₂CO₃, in an appropriate solvent, such as 1,4-dioxane, THF, toluene or 2-ethoxyethanol. In some cases where X′=F, the conversion may be achieved through a S_{N}Ar reaction in the presence of a base, for example DIPEA in an appropriate solvent like DMF.

A further deprotection step may be required for some specific examples. Such transformations are well-known to those of skill in the art. For example, in the cases of B′ ring is 2-alkoxy-azaheterocycle, the alkoxy protecting group can be removed by procedure described in Scheme VI′.

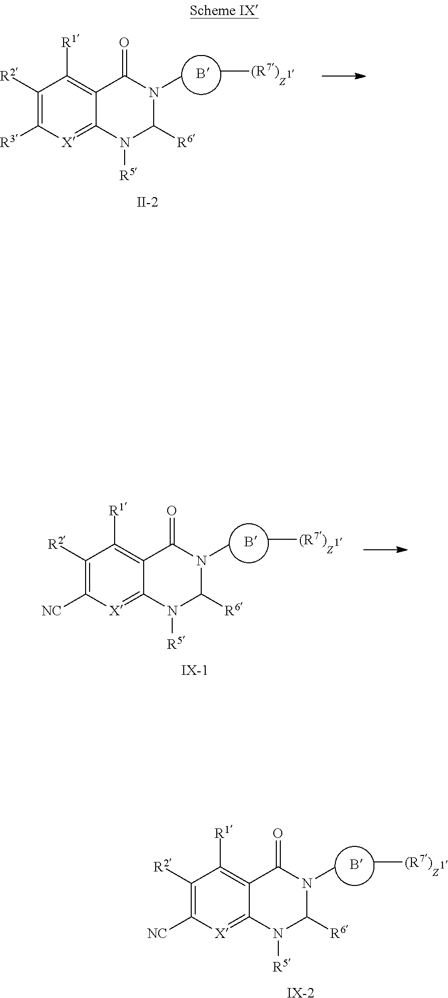

In the instance where R^{3′} in II-2 is substituted with fluoro- group (R^{3′}=F), the fluoro group can be replaced with cyano group through the reaction between II-2 and sodium cyanide in the presence of tetrabutylammonium bromide in an appropriate polar solvent, typically DMSO, at elevated temperature.

Alternatively, for compound II-2 with R^{3′} is bromo- or iodo- group, the conversion from the halogen to cyano group can be achieved by treating II-2 with copper(I) cyanide in DMF at elevated temperature. This method may also apply to the conversion of bromo group at other positions on the ring to cyano group, such as at R^{2′}.

As necessary, the final compound IX-2 can be generated from IX-1 via appropriate deprotection reaction or suitably methods illustrated in Scheme V′ to VIII′. The selection of reactions and the corresponding conditions are apparent to those of skill in the art.

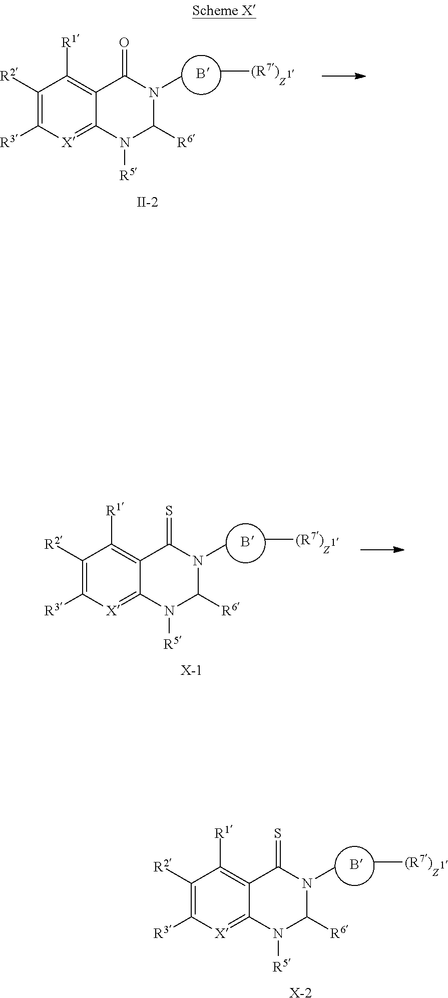

The intermediate dihydroquinazolinone II-2 can be converted to dihydroquinazolinthione X-1 by reacting with a thiation agent for instance Lawesson's reagent at elevated temperature in an appropriate solvent such as toluene. Since B′ ring is 2-alkoxy-azaheterocycle in this example, the alkoxy protecting group can be removed by procedure described in Scheme V to provide final compound X-2.

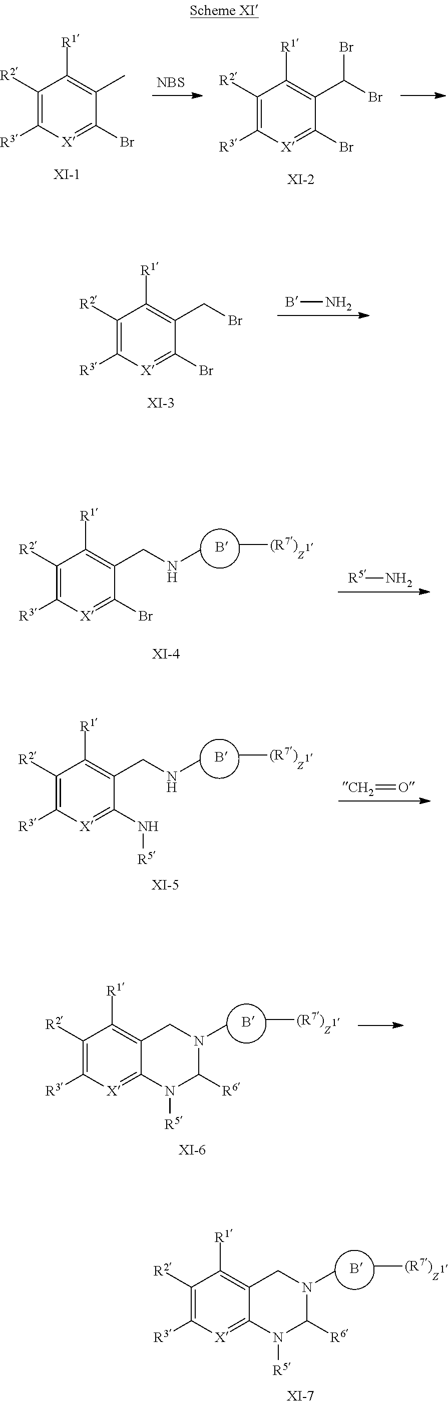

XI-4′ is the key intermediate to prepare tetrohydroquinazolinone compound XI-7. Bromination of bromosubstituted methyl benzene XI-1 with NBS in presence of benzoyl peroxide provides dibromomethyl benzene XI-2. Monobromomethyl benzene XI-3 can be prepared from XI-2 by using diethyl phosphate in presence of organic base DIEA in an appropriate solvent such as THF. Coupling of XI-3 with a suitable 2-alkoxy-azaheterocycle B′—NH₂, for example 2-methoxy-4-aminopyridine, in the presence of cesium carbonate at elevated temperature in an appropriate solvent such as acetonitrile, provides the corresponding amine XI-4. Using chemistry similar to that described in Schemes I′, II′, and V′, compound XI-7 can be readily prepared from XI-4.

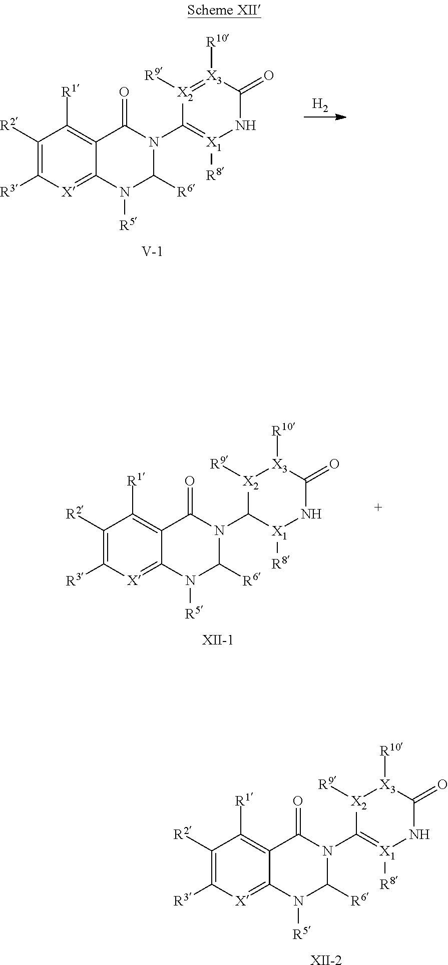

In the instance where B′ ring in the final compound is hydropyridone product such as XII-1 or XII-2, it may be prepared from the corresponding pyridone V-1 through hydrogenation. The reaction can be completed using Platinum on charcoal as catalyst in a suitable solvent such as ethanol at room temperature and ATM pressure. This procedure generates two products, piperidone XII-1 and dihydropyrindone XII-2.

| | Equivalent Substituents In Formulas (I) to (III) |
|---|---|
| Compounds/Substituents: Schemes I To VII | (inc. subgeneric formulas defined herein): |
| 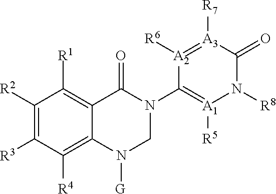 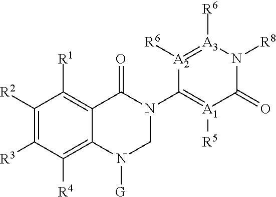 | 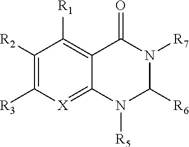 X = —N or —C—R₄ (I) |
| Substituent “G” | Equivalent to Substituent R⁵ which is: |
| | unsaturated or saturated carbocyclic ring, |
| | —CH₂-unsaturated carbocyclic ring, |
| | unsaturated or saturated heterocyclic or heteroaryl ring |
| Substituent identified as: | Equivalent to Substituent R7 is: |
| 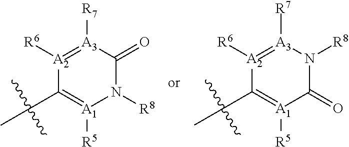 A₁, A₂ or A₃ is N or C R₆, R₇, R₈ = as defined below | 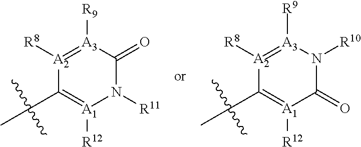 unsubstituted or substituted saturated monocyclic heterocyclic ring: 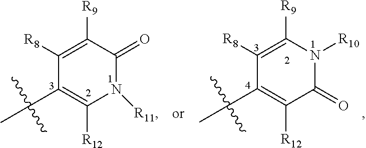 |
| | 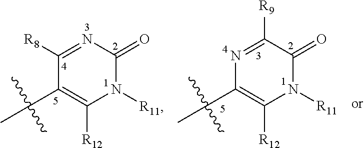 |
| | 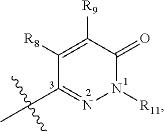 A₁, A₂ or A₃ is N or C R₆, R₇, R₈ = R₈, R₉, R₁₀, R₁₁, R₁₂ accordingly |

### General Methods of Preparation

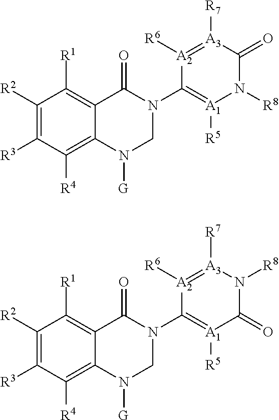

The compounds of the present invention may be obtained by using the procedures illustrated in the Schemes below, or by applying appropriate synthetic organic chemistry procedures and methodology known to those of skill in the art.

The methods provided in these Schemes can be used to prepare compounds of the invention containing a variety of different R¹, R², R³, R⁴, R⁵, R⁶, R⁷ and G groups employing appropriate precursors.

Those skilled in the art will appreciate that in the preparation of compounds of the invention (e.g., any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) pharmaceutically acceptable salts, solvates, hydrates thereof and the like), it may be necessary and/or desirable to protect one or more sensitive groups in the molecule or the appropriate intermediate to prevent undesirable side reactions.

Suitable protecting groups for use according to the present invention are well-known to those skilled in the art and may be used in a conventional manner. See for example, “Protective Groups in Organic Synthesis” by T. W. Green and P. G. M Wets (Wiley & Sons, 1991) or “Protecting Groups” by P. J. Kocienski (Georg Thieme Verlag, 1994). Subsequent deprotection, where needed, affords compounds of the nature generally disclosed. While the Schemes shown below are representative of methods for preparing compounds of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) of the present invention, they are only intended to be illustrative of processes that may be used to make the compounds of the invention.

Compound names were generated using the software naming program ChemDraw Ultra v12.0, available from Perkin Elmer, 940 Winter Street, Waltham, Mass., 02451, USA. (http://www.perkinelmer.com).

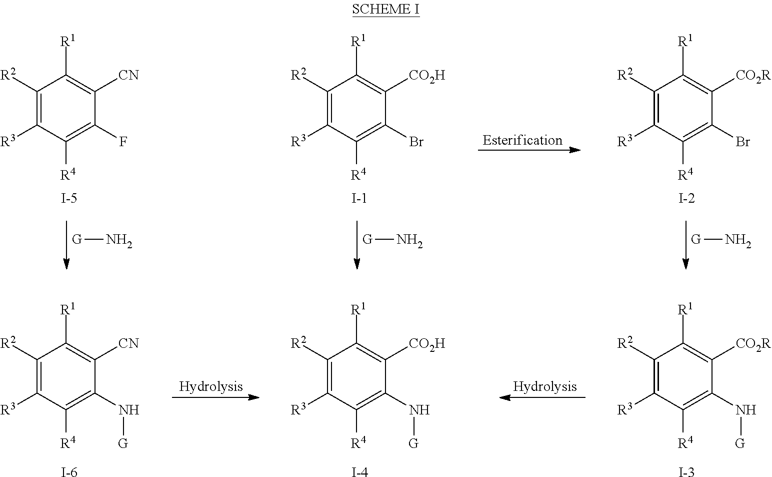

The preparation of the compounds of the present invention typically begins with the synthesis of N-substituted-2-aminoaromatic acid derivatives I-4 (Scheme I). Esterification of a suitably substituted 2-bromoaromatic acid under standard conditions provides the corresponding ester I-2. Typically, esterification reactions are performed under either acidic conditions, in the presence of an alcohol, or under basic conditions, in the presence of a suitable alkyl halide. Such conditions are well-known to those of skill in the art. Reaction of the 2-bromoaromatic ester I-2 with an appropriate aniline or amine (G-NH₂) provides the corresponding N-substituted-2-aminoaromatic esters I-3. Typically, this reaction is performed at elevated temperature, using either standard heating or microwave irradiation, in the presence of a catalyst, for example Pd₂(dba)₃ or Cu/CuO, a suitable ligand, for instance BINAP or Xantphos, and an inorganic base, typically Cs₂CO₃ or K₂CO₃, in an appropriate solvent, such as 1,4-dioxane, toluene or 2-ethoxyethanol. Saponification of the ester I-3 to the corresponding N-substituted-2-aminoaromatic acid derivatives (I-4) is typically achieved under standard basic conditions, using bases such as LiOH, KOH, or NaOH, in a suitable solvent or solvent system, for instance methanol/H₂O, ethanol/H₂O, or THF/H₂O. Such conditions are well-known to those of skill in the art.

An alternative approach, which will be readily apparent to those of skill in the art, is to react the 2-bromoaromatic acid I-1 with an appropriate aniline or amine (G-NH₂) to provide compound I-4 directly. The reaction conditions are similar to those described above for conversion of I-2 to I-3, but use of a ligand may, or may not, be necessary.

In another alternative approach, the intermediate N-substituted-2-aminoaromatic acid derivatives I-4 can be prepared starting from suitably substituted 2-fluoro-aromatic nitriles I-5, using an S_{N}Ar reaction. For example, compound I-5 can be reacted with an appropriate aniline or amine (G-NH₂) to effect the desired S_{N}Ar reaction to afford the substitution product I-6. This reaction is typically conducted in the presence of a base, oftentimes NaH or K₂CO₃, in an appropriately polar solvent such as DMF or DMSO. This reaction can be done at either room temperature or with heating, depending on the relative reactivity of the starting materials. The nitrile group of I-6 is then hydrolyzed to the corresponding carboxylic acid I-4 by reaction with a hydroxylic base, usually LiOH, KOH, or NaOH, in a suitable solvent or solvent system, for instance methanol/H₂O, ethanol/H₂O, or THF/H₂O. This reaction can be done at either room temperature or with heating. Such conditions are well-known to those of skill in the art.

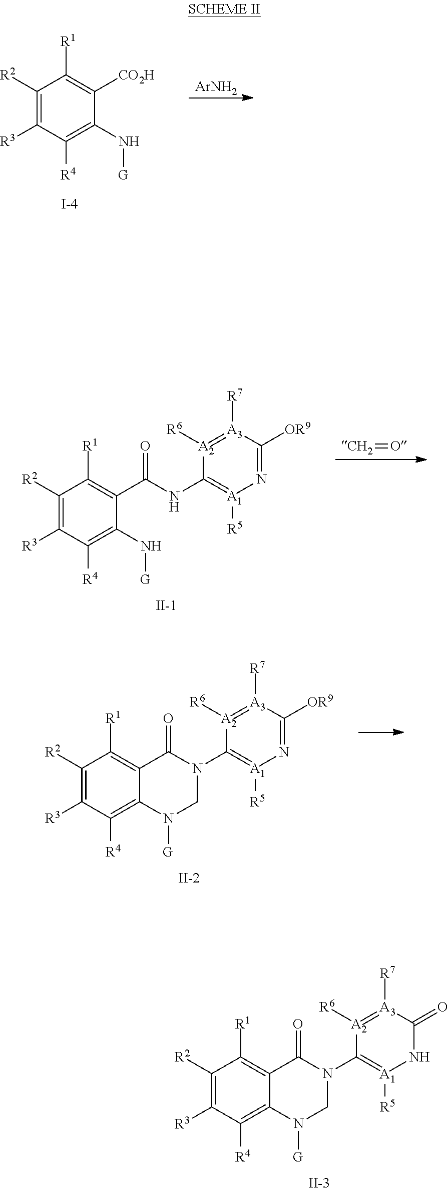

The intermediate N-substituted-2-aminoaromatic acid derivatives I-4, prepared as illustrated in Scheme I, can be converted to the compounds of the present invention as outlined in Scheme II. The synthesis of compounds like II-3 is illustrative of the method. Coupling of I-4 with a suitable 2-alkoxy-azaheterocycle, for example 2-methoxy-4-aminopyridine, under various amide couple conditions known to those of skill in the art, provides the corresponding amide II-1. For example, one might employ standard coupling reagents, like EDC/HOBT, HATU or HBTU, in the presence of an amine base, like triethylamine, or Hunig's base (diisopropylethylamine), in a suitable solvent, typically DMF, DMA or acetonitrile. Alternatively, one might convert the acid to the corresponding acid chloride, using a reagent like thionyl chloride or oxalyl chloride, then react the acid chloride with a suitable 2-alkoxy-azaheterocycle (like 2-methoxy-4-aminopyridine), in the presence of an acid scavenger or base, such as pyridine, 2,6-lutidine, triethylamine or Hunig's base, in an appropriate solvent, such as dichloromethane or pyridine, to afford the desired coupling product II-1. It will be obvious to those of skill in the art that use of 2-methoxy-4-aminopyridine as the coupling partner, as illustrated in Scheme II, is representative of a general method, and that other amino heterocycles can be used. Formation of the 2,3-dihydroquinazolin-4(1H)-one ring system, as in II-2, involves reaction of II-1 with formaldehyde or a suitable equivalent. For instance, the reaction may be achieved using formaldehyde, either as gaseous formaldehyde, paraformaldehyde, or s-trioxane, in the presence of an acid, oftentimes pyridinium p-toluenesulfonate (PPTS) or p-toluenesulfonic acid. The reaction is typically conducted at elevated temperature, using toluene or 2,2,2-trifluoroethanol as the solvent. Alternatively, the 2,3-dihydroquinazolin-4(1H)-one ring system can be formed via reaction of II-1 using diiodomethane as a formaldehyde equivalent. In this variant of the cyclization reaction, a base, typically Cs₂CO₃, is used, in a suitable solvent, oftentimes acetonitrile. The choice of using formaldehyde or diiodomethane depends on the particular reactivity characteristics of the substrate II-1. To complete the synthesis of the compounds of the present invention, removal of the alkoxy (typically methoxy) protecting group from the 2-alkoxy-azaheterocycle II-2 may be required. Preferred methods for achieving this transformation include reaction with a mixture of TMS-chloride and NaI, or a solution of TMS-iodide, in a neutral solvent like acetonitrile, at elevated temperature. Alternatively, this conversion may be achieved utilizing a mixture of p-toluenesulfonic acid and LiCl in a solvent such as DMF at elevated temperature.

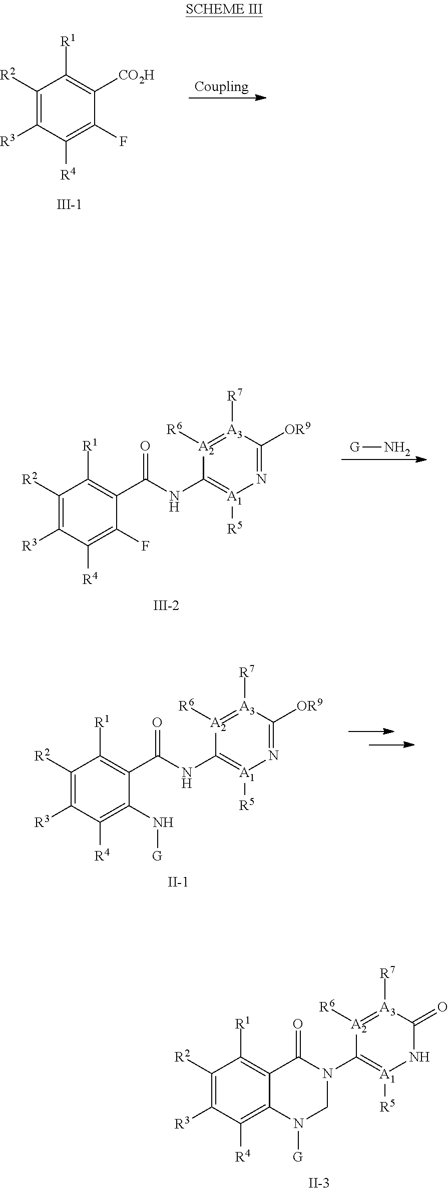

In a variation of the methods described in Schemes I and II, the compounds of the present invention can be prepared as illustrated in Scheme III. Coupling of III-1 with a suitable 2-alkoxy-azaheterocycle, for example 2-methoxy-4-aminopyridine, under various amide couple conditions known to those of skill in the art, provides the corresponding amide III-2. General conditions for forming amides are described in Scheme II. Subsequently, compound III-2 can be reacted with an appropriate aniline or amine (G-NH₂) to effect an S_{N}Ar reaction to afford product II-1. This reaction is typically conducted in the presence of a base, for instance LiHMDS or NaH, in a neutral solvent such as THF. This reaction can be done at either room temperature or with heating, depending on the relative reactivity of the 2-fluoro-aromatic carboxylic acid starting material. Compound II-1 can then be converted to compound II-3 according to the methods illustrated in Scheme II.

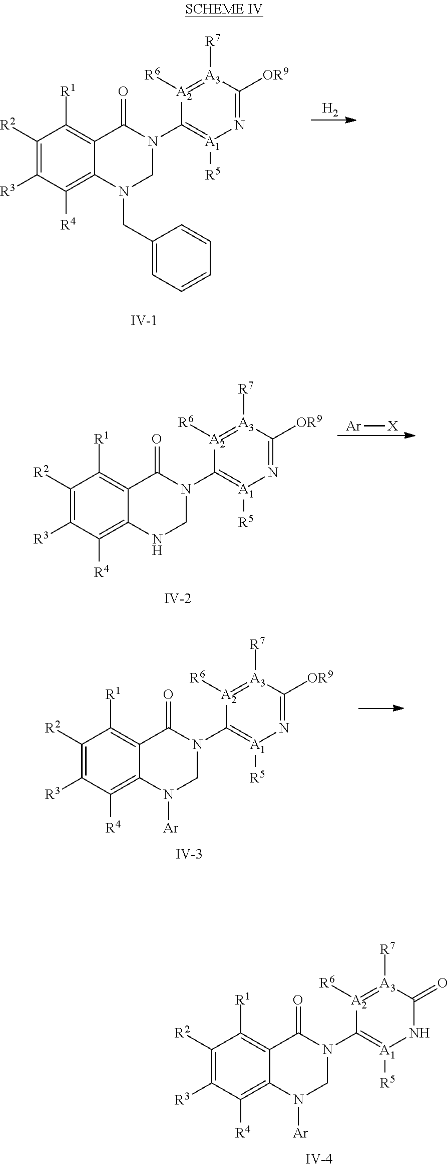

An alternative method for preparing the compounds of the present invention is shown in Scheme IV. Using chemistry similar to that described in Schemes I, II, and III, compounds like IV-1 can be readily prepared. N-Debenzylation of IV-1 may be achieved via hydrogenation using a catalyst such as Pd/C and a hydrogen source (e.g. hydrogen gas or ammonium formate), to afford the corresponding N-debenzylated derivatives IV-2. These compounds can be N-arylated via reaction with aryl halides under appropriate conditions. For example, IV-2 can react with a suitably functionalized aryl chloride, bromide or iodide, in the presence of a catalyst, for example Pd₂(dba)₃, Cu₂O, or Cu/CuO, and a suitable ligand, for instance BINAP or Xantphos. The reaction is typically conducted at elevated temperature in the presence of an inorganic base, typically NaOtBu, Cs₂CO₃ or K₃PO₄, in an appropriate solvent, such as 1,4-dioxane, toluene or DMSO. With certain aryl halides, IV-2 can be induced to participate in a nucleophilic aromatic substitution reaction to provide IV-3. For example, reaction of IV-2 with certain 2-haloheterocycles, in the presence of a base like Cs₂CO₃, NaH or LiHMDS, in a neutral solvent like THF, NMP or DMF, and typically at elevated temperature, can provide the N-aryl derivatives IV-3. Compounds IV-3 can be converted to the final compounds as illustrated in Scheme II.

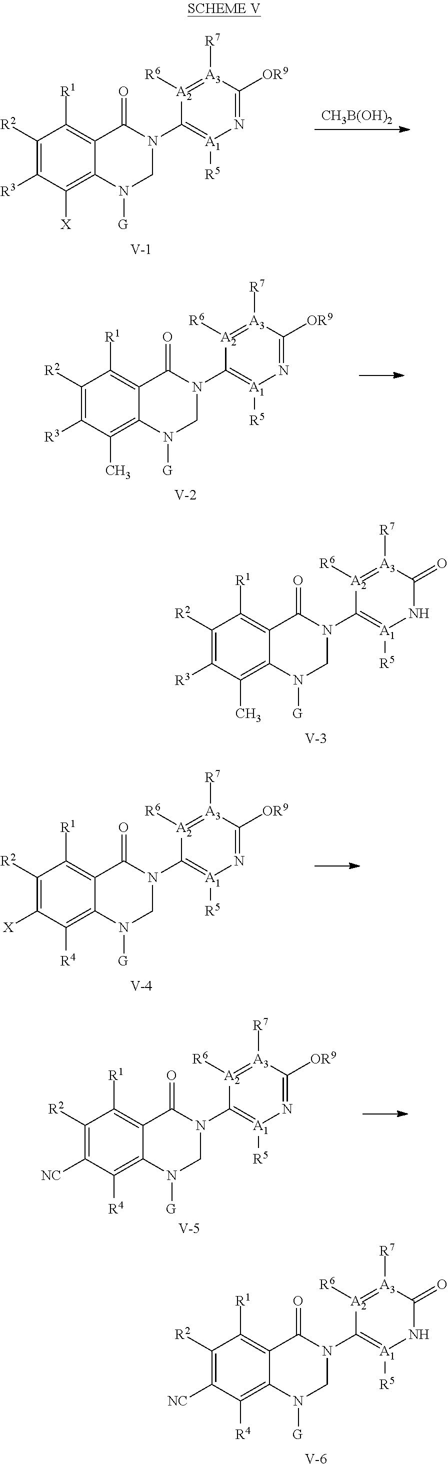

In the instance where intermediates are substituted by appropriate halogens, particularly chlorine, bromine, or iodine (X=Cl, Br or I), another method can be used to prepare selected compounds of this invention. For example, as illustrated in Scheme V, compound V-1, where X=Cl, Br or I, prepared as illustrated in previous Schemes, can be converted to a methyl-substituted derivative such as V-2. This transformation typically involves reaction with a suitable boronic acid or ester, for instance CH₃B(OH)₂, in the presence of a palladium catalyst and a suitable ligand. Oftentimes, Pd(OAc)₂ is the catalyst of choice, and ligands such as Cy₃P.HBF₄ and Xphos are preferred. Reactions like this are typically performed at elevated temperature, in the presence of a suitable inorganic base, such as Cs₂CO₃ or K₃PO₄, in a neutral solvent or solvent mixture, such as toluene/H₂O. Similarly, compound V-4, where X=Cl, Br or I, prepared as illustrated in previous Schemes, can be converted to a cyano-substituted derivative such as V-5. For example, V-4 can react with Zn(CN)₂ in the presence of a catalyst, such as Pd(OAc)₂, and a suitable ligand, for example Xphos. The reaction is typically conducted at elevated temperature, in the presence of an acid such as HCl or H₂SO₄, with a stoichiometric amount of Zn powder, in an appropriate solvent, such as DMA. Those of skill in the art will recognize that the procedures illustrated in Scheme V are applicable to the incorporation of methyl or cyano groups at any of the available R¹-R⁷ positions. Compounds V-2 and V-5 can be converted to the final compounds V-3 and V-6, respectively, by the general methods described in Scheme II.

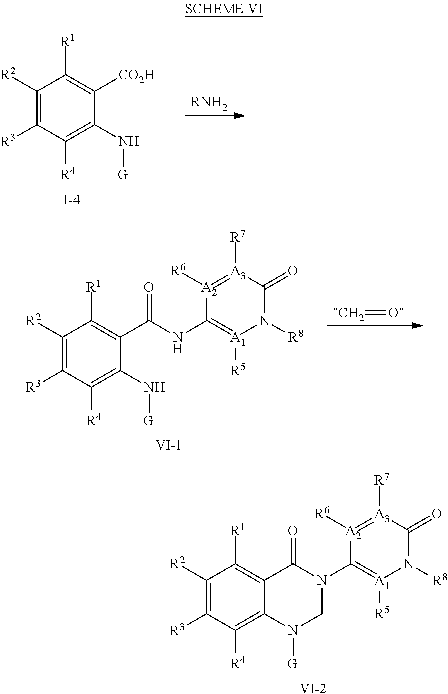

Compounds of the present invention, wherein R⁸ is an alkyl group can be prepared as illustrated in Scheme VI. Compound I-4, prepared according to the methods shown in Scheme I, can be coupled with a 1-alkylated aminopyridone, for instance 5-amino-1-methylpyridin-2(1H)-one, under various amide couple conditions known to those of skill in the art, to afford the corresponding amide VI-1. General conditions for forming amides are described in Scheme II. It will be obvious to those of skill in the art that use of 5-amino-1-methylpyridin-2(1H)-one as the coupling partner, as illustrated in Scheme VI, is representative of a general method, and that other 1-alkylated aminopyridones can be used. Compound VI-1 can subsequently be cyclized to the corresponding 2,3-dihydroquinazolin-4(1H)-one according to the methods described in Scheme II.

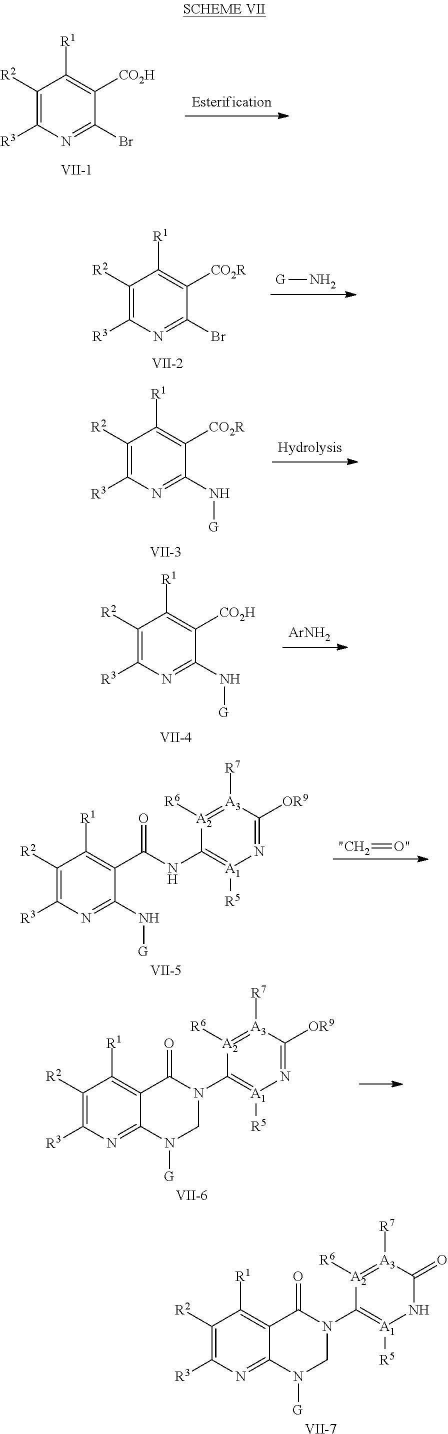

Compounds of the present invention wherein X is N can be prepared as illustrated in Scheme VII. Using chemistry similar to that described in Schemes I and II, compounds like VII-7 can be readily prepared. Esterification of a suitably substituted 2-bromopyridine acid VII-1 under standard conditions provides the corresponding ester VII-2. General esterification conditions are described in Scheme 1. Reaction of the 2-bromopyridine ester VII-2 with an appropriate aniline or amine (G-NH₂) provides the corresponding N-substituted-2-aminopyridine esters VII-3. The conditions for this reaction are described in Scheme 1. Saponification of the ester VII-3 to the corresponding N-substituted-2-aminopyridine acid derivatives (VII-4) is described in Scheme 1. Coupling of VII-4 with a suitable 2-alkoxy-azaheterocycle, for example 2-methoxy-4-aminopyridine, under various amide couple conditions known to those of skill in the art, provides the corresponding amide VII-5. General conditions for forming amides are described in Scheme II. Compound VII-5 can subsequently be cyclized to the corresponding dihydroquinazolinone and to the final compound VII-6 according to the methods described in Scheme II.

Pharmaceutical Compositions, Formulations. Dosage Forms

The present invention relates to pharmaceutical compositions, formulations or dosage forms and the like, comprised of:
[a] novel compounds of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) of the present invention; and
[b] at least one pharmaceutically acceptable excipient(s).

The compounds of the invention will normally, but not necessarily, be formulated into a pharmaceutical composition, formulations or dosage forms and the like prior to administration to a patient.

Accordingly, the present invention is directed to pharmaceutical compositions, formulations, dosage forms and the like as defined therein, which comprise a compound or compound species of the present invention (i.e., as defined throughout the present application) and pharmaceutically-acceptable excipient(s).

In particular, the present invention also may relate to a pharmaceutical composition or formulation, which comprises:
[a] a compound as defined by any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined herein) of the present invention;
[b] at least one pharmaceutically acceptable excipient(s), and
[c] optionally one or more other therapeutic ingredients.

The pharmaceutical compositions of the invention may be prepared and packaged in bulk form wherein an effective amount of a compound of the invention can be extracted and then given to the patient such as with powders, syrups, and solutions for injection. Alternatively, the pharmaceutical compositions of the invention may be prepared and packaged in unit dosage form.

For oral application, for example, one or more tablets or capsules may be administered. A dose of the pharmaceutical composition contains at least a therapeutically effective amount of a compound of this invention (i.e., any of the Formulas disclosed herein, including a compound of Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) of the present invention, particularly a pharmaceutically acceptable salt, thereof).

The pharmaceutical compositions or formulations as defined herein typically contain one compound of the present invention. However, in certain embodiments, the pharmaceutical compositions may contain more than one compound of the present invention. In addition, the pharmaceutical compositions of the present invention may optionally further comprise one or more additional pharmaceutically active compounds.

As used herein, “pharmaceutically-acceptable excipient” means a material, composition or vehicle involved in giving form or consistency to the composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically-acceptable are avoided. In addition, each excipient must of course be of sufficiently high purity to render it pharmaceutically-acceptable.

Suitable pharmaceutically-acceptable excipients will vary depending upon the particular dosage form chosen. In addition, suitable pharmaceutically-acceptable excipients may be chosen for a particular function that they may serve in the composition.

For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Moreover, pharmaceutical compositions, formulations, dosage forms, and the like, etc. may conveniently be presented in unit dosage form and may be prepared by any of the methods well known in the art of pharmacy.

All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The compounds of the invention and the pharmaceutically-acceptable excipient or excipients will typically be formulated into a dosage form adapted for administration to the patient by the desired route of administration.

With regard to the present invention, conventional dosage forms include those adapted for:

(1) oral administration such as tablets, capsules, caplets, pills, troches, powders, syrups, elixirs, suspensions, solutions, emulsions, sachets, and cachets;

(2) parenteral administration such as sterile solutions, suspensions, and powders for reconstitution;

(3) transdermal administration such as transdermal patches;

(4) rectal administration such as suppositories;

(5) inhalation such as aerosols and solutions; and

(6) topical administration such as creams, ointments, lotions, solutions, pastes, sprays, foams, and gels.

The pharmaceutical compositions or formulations of the invention are prepared using techniques and methods known to those skilled in the art. Some of the methods commonly used in the art are described in Remington's Pharmaceutical Sciences (Mack Publishing Company).

In general, pharmaceutical compositions of the present invention are prepared using conventional materials and techniques, such as mixing, blending and the like.

The term “active agent” is defined for purposes of the present invention as any chemical substance or composition of the present invention, which can be delivered from the device into an environment of use to obtain a desired result.

The percentage of the compound in compositions can, of course, be varied as the amount of active in such therapeutically useful compositions is such that a suitable dosage will be obtained.

In another aspect, the present invention relates to a pharmaceutical composition comprising a compound of any of the Formulas disclosed herein, including Formula (I) or a pharmaceutically acceptable salt and/or a corresponding tautomer form thereof and at least one or more pharmaceutically acceptable excipients.

In another aspect, the present invention relates to a pharmaceutical composition or formulation, which comprises:
a compound of any of the Formulas disclosed herein, including Formula (I) or a pharmaceutically acceptable salt and/or a corresponding tautomer form thereof; and
at least one or more pharmaceutically acceptable excipient(s).

In another aspect, the present invention relates to a pharmaceutical composition or formulation, which comprises:
[a] a compound of the present invention according to any one of the Formulas identified below (i.e., which are defined throughout the instant application):
   Formulas (I), (IA) or (IB);
   Formulas (II), (IA) or (IIB);
   Formulas (III), (IIIA) or (IIIA″);
   Formulas (IIIB) or (IIIB′);
   Formulas (IIIC) or (IIIC′);
   Formulas (IIID);
   Formulas (IIIE) or (IIIE′); or
      a pharmaceutically acceptable salt and/or a corresponding tautomer form thereof; and
[b] at least one or more pharmaceutically acceptable excipient(s).

In another aspect, the present invention relates to a pharmaceutical composition or formulation, which comprises:
[a] any of the compounds of the present invention, which may include any of the compound intermediates, compound species or Examples defined in the instant application; or a pharmaceutically acceptable salt and/or a corresponding tautomer form thereof; and
[b] at least one or more pharmaceutically acceptable excipient(s).

It will be appreciated that the actual preferred dosages of the compounds being used in the compositions of this invention will vary according to the particular composition formulated, the mode of administration, the particular site of administration and the host being treated.

The active compounds of the present invention may be orally administered, for example, with an inert diluent, or with an assimilable edible carrier, or they can be enclosed in hard or soft shell capsules, or they can be compressed into tablets, or they can be incorporated directly with the food of the diet, etc.

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

Where appropriate, dosage unit formulations for oral administration can be microencapsulated. The composition can also be prepared to prolong or sustain the release as for example by coating or embedding particulate material in polymers, wax or the like.

The compounds of the invention may also be coupled with soluble polymers as targetable drug carriers. Such polymers can include polyvinylpyrrolidone, pyran copolymer, polyhydroxypropylmethacrylamide-phenol, polyhydroxyethyl aspartamide phenol, or polyethyleneoxidepolylysine substituted with palmitoyl residues. Furthermore, the compounds of the invention may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Methods, Use(s), Compounds for Use in Manufacture and/or Treatment of Diseases

In general, the present invention relates to method(s), use(s) in therapy, or compound(s) for use in manufacturing a medicament and/or or treating:

pain and/or pain-associated disease(s), disorder(s) or condition(s), respectively, which comprises administering a therapeutically effective amount of:
a compound of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) or a pharmaceutically acceptable salt thereof; or
a corresponding pharmaceutical composition or formulation thereof to a patient or subject in need thereof.

As used herein, “patient” or “subject” in need thereof refers to a human or mammal. Suitably the subject being treated is a human.

In another aspect, the present invention also relates to method(s) for treating, use(s) in therapy, compound(s) for use in manufacturing a medicament and/or for treating:
pain-associated disease(s), disorder(s) or condition(s), such as pain caused by a variety of diseases as defined herein in the present application;
pain caused by trauma;
pain caused by iatrogenic (i.e., such as medical or dental) procedures;
atrial fibrillation that is either idiopathic in nature or caused by a variety of diseases as defined herein in the present application.

As used herein, the term “iatrogenic” refers to pain induced inadvertently by a medical or dental personnel, such as surgeon or dentist, during medical or dental treatment(s) or diagnostic procedure(s), which include, but are not limited to pain caused by pre-operative (i.e., “before”), peri-operative (i.e., “during” or medically induce pain during non-surgical or operative treatment(s)) and post-operative (i.e., after, post-operative or surgical induced caused pain) medical or dental procedures.

In another aspect, the present invention relates to method(s) for treating, use(s) in therapy, compound(s) for use in manufacturing a medicament and/or for treating:
pain-associated disease(s), disorder(s) or condition(s);
pain caused by trauma;
pain caused by iatrogenic, medical or dental procedures; or
idiopathic atrial fibrillation or caused by associated disease(s), disorder(s) or condition(s);
   which comprises administering a therapeutically effective amount of any compound of the present invention or a pharmaceutically acceptable salt and/or a corresponding tautomer form thereof; or a pharmaceutical composition or formulation thereof to a patient or subject in need thereof.

In another aspect, the present invention relates to method(s) for treating, use(s) in therapy, compound(s) for use in manufacturing a medicament and/or for treating:
the pain-associated disease(s), disorder(s) or condition(s);
the pain caused by trauma;
the pain caused by iatrogenic, medical or dental procedures, respectively; or
idiopathic atrial fibrillation or caused by associated disease(s), disorder(s) or condition(s);
   is selected from:
chronic pain;
acute pain;
neuropathic pain;
inflammatory pain of varied physiologic origins;
nociceptive pain;
neurologic, neuropathy or neuronal injury associated or related pain disorders caused by diseases; neuralgias and associated acute or chronic pain;
post-herpetic neuralgia;
musculoskeletal pain; lower back and neck pain; sprains and strains;
myofascial pain; myositis or muscle inflammation;
repetitive motion pain;
complex regional pain syndrome;
chronic or acute arthritic pain;
sympathetically maintained pain;
cancer, toxins and chemotherapy related pain;
postsurgical pain syndromes and/or associated phantom limb pain;
post-operative medical or dental procedures or treatments pain;
pain associated with HIV, pain induced by HIV treatment;
paroxysmal atrial fibrillation;
sustained atrial fibrillation;
long-standing atrial fibrillation;
atrial fibrillation with heart failure;
atrial fibrillation with cardiac valve disease; or
atrial fibrillation with chronic kidney disease.

In another aspect, the present invention relates to these definitions of pain as follows:
nociceptive pain is selected from post-surgical pain, cancer pain, back and craniofacial pain, osteoarthritis pain, dental pain or diabetic peripheral neuropathy;
inflammatory pain is selected from pain associated with rheumatoid arthritis, osteoarthritis, rheumatoid spondylitis, gouty arthritis or juvenile arthritis;
musculoskeletal pain selected from bone and joint pain, osteoarthritis; lower back and neck pain; pain resulting from physical trauma or amputation;
neurologic or neuronal injury associated or related pain disorders caused by diseases selected from neuropathy, pain associated nerve injury, pain associated root avulsions, painful traumatic mononeuropathy, painful polyneuropathy, erythromelalgia, paroxysmal extreme pain disorder (PEPD), burning mouth syndrome; central pain syndromes caused by a lesion at a level of nervous system); traumatic nerve injury, nerve compression or entrapment, congenital insensitivity to pain (CIP), dysmenorrheal, primary erythromelalgia; HIV peripheral sensory neuropathy; pudendal neuralgia, spinal nerve injury, chronic inflammatory demyelinating polyneuropathy (CIDP), carpal tunnel syndrome or vasculitic neuropathy; or
inflammatory pain of varied origins selected from: osteoarthritis, rheumatoid arthritis, rheumatic disorder, teno-synovitis and gout, shoulder tendonitis or bursitis, gouty arthritis, and polymyalgia rheumatica, primary hyperalgesia, secondary hyperalgesia, primary allodynia, secondary allodynia, or other pain caused by central sensitization; complex regional pain syndrome, chronic arthritic pain and related neuralgias, acute pain, or atrial fibrillation selected from:
paroxysmal atrial fibrillation, sustained atrial fibrillation, long-standing atrial fibrillation, atrial fibrillation with heart failure, atrial fibrillation with cardiac valve disease, or atrial fibrillation with chronic kidney disease.

In one aspect, the present invention relates to:
the pain-associated disease(s), disorder(s) or condition(s);
the pain caused by trauma; or
the pain caused by iatrogenic, medical or dental procedures, respectively
idiopathic atrial fibrillation or caused by associated disease(s), disorder(s) or condition(s);
   selected from:
chronic, acute or pre-operative associated pain;
acute, chronic or post-operative associated pain; or
paroxsymal, sustained or long-standing atrial fibrillation.

In another aspect, the present invention relates to method(s) for treating, use(s) in therapy, compound(s) for use in manufacturing a medicament and/or for treating:
chronic, acute or pre-operative associated pain is selected from neuropathic pain or chronic neuropathic pain, chronic osteoarthritis pain, dental pain or inflammatory pain;
acute, chronic or post-operative associated pain is selected from bunionectomy pain, hernia repair pair, breast surgery pain or cosmetic surgical pain; or
atrial fibrillation selected from paroxysmal, sustained or long-standing.

In another aspect, the present invention relates to method(s) for treating, use(s) in therapy, compound(s) for use in manufacturing a medicament and/or for treating:
neuropathic pain or chronic neuropathic pain is selected from small fiber-mediated diabetic neuropathy, small fiber neuropathy, idiopathic small fiber neuropathy, painful diabetic neuropathy or polyneuropathy;
inflammatory pain selected from osteoarthritis, chronic osteoarthritis knee pain or chronic inflammatory demyelinating polyneuropathy; or
atrial fibrillation selected from paroxysmal, sustained or long-standing.

In another aspect, the present invention relates to method(s) for treating, use(s) in therapy, compound(s) for use in manufacturing a medicament and/or for treating:
peripheral neuropathic pain, central neuropathic pain, inherited erythromelalgia (IEM), small fiber neuralgia (SFN), paroxysmal extreme pain disorder (PEPD), painful diabetic neuropathy, chronic lower back pain, neuropathic back pain, sciatica, non-specific lower back pain, multiple sclerosis pain, HIV-related neuropathy, post-herpetic neuralgia, trigeminal neuralgia, vulvodynia, pain resulting from physical trauma, post-limb amputation pain, neuroma pain, phantom limb pain, cancer, toxins, chronic inflammatory conditions or atrial fibrillation.

In another aspect, the present invention relates to method(s) for treating, use(s) in therapy, compound(s) for use in manufacturing a medicament and/or for treating inflammatory mediated pain syndromes, which comprises administering a therapeutically effective amount of:
a compound of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) or a pharmaceutically acceptable salt thereof; or
a corresponding pharmaceutical composition or formulation thereof to a patient or subject in need thereof.

In another aspect, the present invention relates to method(s) for treating, use(s) in therapy, compound(s) for use in manufacturing a medicament and/or for treating or reducing severity of:
pain-associated disease(s), disorder(s) or condition(s);
pain caused by trauma;
pain caused by iatrogenic (i.e., such as medical or dental) procedures, respectively; or
paroxysmal, sustained or long-standing atrial fibrillation;
   which comprises administering a therapeutically effective amount of:
a compound of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) or a pharmaceutically acceptable salt thereof; or
a corresponding pharmaceutical composition or formulation thereof
   to a patient or subject in need thereof.

In another aspect, the present invention relates to method(s) for treating, use(s) in therapy, compound(s) for use in manufacturing a medicament and/or for treating or inhibiting a Nav1. 8 voltage-gated sodium channel in a subject comprising administering a therapeutically effective amount of:
a compound of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) or a pharmaceutically acceptable salt thereof; or
a corresponding pharmaceutical composition or formulation thereof
   to a human in need thereof.

In another aspect, the present invention relates to method(s) for treating, use(s) in therapy, compound(s) for use in manufacturing a medicament and/or for treating:
pain and/or pain-associated disorder(s) or condition(s), respectively, selected from:
   chronic, acute or peri or pre-operative associated pain;
   acute, chronic or post-operative associated pain; or
   paroxysmal, sustained or long-standing atrial fibrillation.

In another aspect, the present invention relates to method(s) for treating, use(s) in therapy, compound(s) for use in manufacturing a medicament and/or for treating:
pain and/or pain-associated disease(s), disorder(s) or condition(s), respectively, selected from:
   neuropathic pain, chronic neuropathic pain, chronic osteoarthritis pain, dental pain or inflammatory pain;
   the acute, chronic or post-operative associated pain is selected from: bunionectomy pain, hernia repair pair, breast surgery pain or cosmetic surgical pain; or
   paroxysmal, sustained or long-standing atrial fibrillation.

In another aspect, the present invention relates to method(s) for treating, use(s) in therapy, compound(s) for use in manufacturing a medicament and/or for treating chronic, acute or pre-operative associated pain is selected from:
neuropathic pain or chronic neuropathic pain selected from small fiber-mediated diabetic neuropathy, small fiber neuropathy, idiopathic small fiber neuropathy, painful diabetic neuropathy or polyneuropathy;
inflammatory pain selected from osteoarthritis pain, chronic osteoarthritis knee pain or chronic inflammatory demyelinating polyneuropathy; or
atrial fibrillation selected from paroxysmal, sustained or long-standing.

In another aspect, the present invention relates to method(s) for treating, use(s) in therapy, compound(s) for use in manufacturing a medicament and/or for treating:
neuropathic pain and/or pain-associated disease(s), disorder(s) or condition(s), respectively, comprising administering a therapeutically effective amount of:
   a compound of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) or a pharmaceutically acceptable salt thereof; or
   a corresponding pharmaceutical composition or formulation thereof to a patient or subject in need thereof.

In another aspect, the present invention relates method(s) for treating, use(s) in therapy, compound(s) for use in manufacturing a medicament and/or for treating:
peripheral neuropathic pain, central neuropathic pain, inherited erythromelalgia (IEM), small fiber neuralgia (SFN), paroxysmal extreme pain disorder (PEPD), painful diabetic neuropathy, chronic lower back pain, neuropathic back pain, sciatica, non-specific lower back pain, multiple sclerosis pain, HIV-related neuropathy, post-herpetic neuralgia, trigeminal neuralgia, vulvodynia, pain resulting from physical trauma, post-limb amputation pain, neuroma pain, phantom limb pain, cancer, toxins, chronic inflammatory conditions or atrial fibrillation.

In another aspect, the present invention relates:
method(s) for treating, use(s) in therapy, compound(s) for use in manufacturing a medicament and/or for treating for treating inflammatory pain mediated syndromes, or
methods for treating and reducing severity of:
   pain-associated disease(s), disorder(s) or condition(s), such as pain caused by a variety of diseases as defined herein throughout the instant application;
   pain caused by trauma;
   pain caused by iatrogenic (i.e., such as medical or dental) procedures, respectively; or
   idiopathic atrial fibrillation or caused by associated disease(s), disorder(s) or condition(s);
      which comprises administering a therapeutically effective amount of:
a compound of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) or a pharmaceutically acceptable salt thereof; or
a corresponding pharmaceutical composition or formulation thereof.

In one aspect, the present invention also provides uses of compounds of the invention in the manufacture of a medicament for treating disorders described herein.

In another aspect, the present invention also provides compounds of the invention for use in therapy as described herein or as conventionally understood in the art.

As used herein, “treat” in reference to a condition means:
(1) to ameliorate or prevent the condition or one or more of the biological manifestations of the condition;
(2) to interfere with:
   (a) one or more points in the biological cascade that leads to or is responsible for the condition; or
   (b) one or more of the biological manifestations of the condition,
(3) to alleviate one or more of the symptoms or effects associated with the condition; or
(4) to slow the progression of the condition or one or more of the biological manifestations of the condition.

As indicated above “treatment” of a condition includes prevention of the condition. The skilled artisan will appreciate that “prevention” is not an absolute term.

In medicine, “prevention” is understood to refer to the prophylactic administration of a drug to substantially diminish the likelihood or severity of a condition or biological manifestation thereof, or to delay the onset of such condition or biological manifestation thereof.

As used herein, “effective amount” and “therapeutically effective amount” are used interchangeably. An effective amount in reference to a compound of the invention means an amount of the compound sufficient to treat the patient's condition, but low enough to avoid serious side effects (at a reasonable benefit/risk ratio) within the scope of sound medical judgment.

As used herein, an effective amount of a compound or pharmaceutically acceptable salt of the present invention or corresponding pharmaceutical composition thereof will vary with:
the particular compound chosen (e.g., consider the potency, efficacy, and half-life of the compound);
the route of administration chosen;
the condition being treated; the severity of the condition being treated; the age, size, weight, and physical condition of the patient being treated;
the medical history of the patient being treated;
the duration of the treatment; the nature of concurrent therapy;
the desired therapeutic effect;
like factors; and
   can be routinely determined by the skilled artisan.

In another aspect, the present invention relates to compounds or pharmaceutically acceptable salts of the invention or corresponding pharmaceutical compositions thereof to be useful as inhibitors of voltage-gated sodium channels.

In one aspect, compounds or pharmaceutically acceptable salts of the invention or corresponding pharmaceutical compositions thereof are inhibitors of Nav1. 8 and thus, without wishing to be bound by any particular theory, the compounds and compositions are particularly useful for treating or lessening the severity of a disease, condition, or disorder where activation or hyperactivity of Nav1. 8 is implicated in the disease, condition, or disorder. When activation or hyperactivity of Nav1. 8 is implicated in a particular disease, condition, or disorder, the disease, condition, or disorder may also be referred to as a “Nav1. 8-mediated disease, condition or disorder.”

Accordingly, in another aspect, the present invention provides a method for treating or lessening the severity of a disease, condition, or disorder where activation or hyperactivity of Nav1. 8 is implicated in the disease state. The activity of a compound utilized in this invention as an inhibitor of Nav1. 8 may be assayed according to methods described generally in the Examples herein, or according to methods available to one of ordinary skill in the art.

In another aspect, the present invention relates to method(s) for treating, use(s) in therapy, compound(s) for use in manufacturing a medicament and/or for treating or inhibiting a Nav1. 8 voltage-gated sodium channel in a subject comprising administering a therapeutically effective amount of:
a compound of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) or a pharmaceutically acceptable salt thereof; or
a corresponding pharmaceutical composition or formulation thereof to a human in need thereof.

In another aspect, the invention provides the use of a compound or pharmaceutical composition described herein for the manufacture of a medicament for use in treating or lessening the severity in a subject of chronic pain, gut pain, neuropathic pain, musculoskeletal pain, acute pain, inflammatory pain, cancer pain, idiopathic pain, multiple sclerosis, Charcot-Marie-Tooth syndrome, incontinence or cardiac arrhythmia.

In another aspect, the invention provides the use of a compound or pharmaceutical composition described herein for the manufacture of a medicament for use in treating or lessening the severity in a subject of gut pain, wherein gut pain comprises inflammatory bowel disease pain, Crohn's disease pain or interstitial cystitis pain.

In another aspect, the invention provides the use of a compound or pharmaceutical composition described herein for the manufacture of a medicament for use in a treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma, traumatic neuroma, Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia; post spinal cord injury pain, idiopathic small-fiber neuropathy, idiopathic sensory neuropathy or trigeminal autonomic cephalalgia.

In another aspect, the invention provides the use of a compound or pharmaceutical composition described herein for the manufacture of a medicament for use in treating or lessening the severity in a subject of musculoskeletal pain, wherein musculoskeletal pain comprises osteoarthritis pain, back pain, cold pain, burn pain or dental pain.

In another aspect, the invention the invention provides the use of a compound or pharmaceutical composition described herein for the manufacture of a medicament for use in treating or lessening the severity in a subject of inflammatory pain, wherein inflammatory pain comprises rheumatoid arthritis pain or vulvodynia.

In another aspect, the invention provides the use of a compound or pharmaceutical composition described herein for the manufacture of a medicament for use in treating or lessening the severity in a subject of idiopathic pain, wherein idiopathic pain comprises fibromyalgia pain.

In another aspect, the invention provides the use of a compound or pharmaceutical composition described herein for the manufacture of a medicament in combination with one or more additional therapeutic agents administered concurrently with, prior to, or subsequent to treatment with the compound or pharmaceutical composition.

In another aspect, the invention provides the use of a compound or pharmaceutical composition described herein for the manufacture of a medicament for use in treating or lessening the severity of acute pain, chronic pain, neuropathic pain, inflammatory pain, arthritis, migraine, cluster headaches, trigeminal neuralgia, herpetic neuralgia, general neuralgias, epilepsy, epilepsy conditions, neurodegenerative disorders, psychiatric disorders, anxiety, depression, dipolar disorder, myotonia, arrhythmia, movement disorders, neuroendocrine disorders, ataxia, multiple sclerosis, irritable bowel syndrome, incontinence, visceral pain, osteoarthritis pain, postherpetic neuralgia, diabetic neuropathy, radicular pain, sciatica, back pain, head pain, neck pain, severe pain, intractable pain, nociceptive pain, breakthrough pain, postsurgical pain, cancer pain, stroke, cerebral ischemia, traumatic brain injury, amyotrophic lateral sclerosis, stress induced angina, exercise induced angina, palpitations, hypertension, or abnormal gastro-intestinal motility.

In another aspect, the invention provides the use of a compound or pharmaceutical composition described herein for the manufacture of a medicament for use in treating or lessening the severity of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache, including, cluster headaches; chronic and acute neuropathic pain, post-herpetic neuralgia; diabetic neuropathy; HIV-associated neuropathy; trigeminal neuralgia; Charcot-Marie Tooth neuropathy; hereditary sensory neuropathies; peripheral nerve injury; painful neuromas; ectopic proximal and distal discharges; radiculopathy; chemotherapy induced neuropathic pain; radiotherapy-induced neuropathic pain; post-mastectomy pain; central pain; spinal cord injury pain; post-stroke pain; thalamic pain; complex regional pain syndrome; phantom pain; intractable pain; acute pain, acute post-operative pain; acute musculoskeletal pain; joint pain; mechanical low back pain; neck pain; tendonitis; injury/exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory, burn and trauma pain; acute intermittent pain, endometriosis; acute herpes zoster pain; sickle cell anemia; acute pancreatitis; breakthrough pain; orofacial pain including sinusitis pain, dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease, including, urinary incontinence; hyperactivity bladder; painful bladder syndrome; interstitial cyctitis (IC); prostatitis; complex regional pain syndrome (CRPS), type I and type II; widespread pain, paroxysmal extreme pain, pruritis, tinnitis, or angina-induced pain.

In another aspect, the invention provides the use of a compound or pharmaceutical composition described herein for the manufacture of a medicament for use in treating or lessening the severity of neuropathic pain. In one aspect, the neuropathic pain is selected from post-herpetic neuralgia, diabetic neuralgia, painful HIV-associated sensory neuropathy, trigeminal neuralgia, burning mouth syndrome, post-amputation pain, phantom pain, painful neuroma, traumatic neuroma, Morton's neuroma, nerve entrapment injury, spinal stenosis, carpal tunnel syndrome, radicular pain, sciatica pain, nerve avulsion injury, brachial plexus avulsion, complex regional pain syndrome, drug therapy induced neuralgia, cancer chemotherapy induced neuralgia, anti-retroviral therapy induced neuralgia, post spinal cord injury pain, idiopathic small-fiber neuropathy, idiopathic sensory neuropathy or trigeminal autonomic cephalalgia.

### Administration

Treatment regimen for the administration of compounds of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) of the present invention or corresponding pharmaceutical compositions of the present invention also may be determined readily by those with ordinary skill in art.

The quantity of the compound, pharmaceutical composition, or dosage form of the present invention administered may vary over a wide range to provide in a unit dosage in an effective amount based upon the body weight of the patient per day to achieve the desired effect and as based upon the mode of administration.

The scope of the present invention includes all compounds, pharmaceutical compositions, or controlled-release formulations or dosage forms, which is contained in an amount effective to achieve its intended purpose. While individual needs vary, determination of optimal ranges of effective amounts of each component is within the skill of the art.

In one aspect:
compounds of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) or a pharmaceutically acceptable salt thereof; or
a corresponding pharmaceutical composition or formulation thereof,
   of the present invention may be administered by any suitable route of administration, including both systemic administration and topical administration.

Systemic administration includes oral administration, parenteral administration, transdermal administration, rectal administration, and administration by inhalation.

Parenteral administration refers to routes of administration other than enteral, transdermal, or by inhalation, and is typically by injection or infusion. Parenteral administration includes intravenous, intramuscular, and subcutaneous injection or infusion.

Inhalation refers to administration into the patient's lungs whether inhaled through the mouth or through the nasal passages. In one aspect, pharmaceutical compositions, formulations, dosages, dosage forms or dosing regimens of the present invention are adapted for administration by inhalation.

Topical administration includes application to the skin as well as intraocular, intravaginal, and intranasal administration.

The present invention as defined herein and throughout the instant application may be administered once or according to a dosing regimen, where a number of doses are administered at varying intervals of time for a given period of time. For example, doses may be administered one, two, three, or four times per day. Doses may be administered until the desired therapeutic effect is achieved or indefinitely to maintain the desired therapeutic effect.

Suitable dosing regimens for compounds of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) of the present invention or corresponding pharmaceutical compositions of the present invention depend on the pharmacokinetic properties of that compound, such as absorption, distribution, and half-life, which can be determined by the skilled artisan.

In addition, suitable dosing regimens, including the duration such regimens are administered, for a compound of the invention depend on the condition being treated, the severity of the condition being treated, the age and physical condition of the patient being treated, the medical history of the patient being treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

In another aspect, the invention is directed to a liquid oral dosage form. Oral liquids such as solution, syrups and elixirs can be prepared in dosage unit form so that a given quantity contains a predetermined amount of a compound of the invention. Syrups can be prepared by dissolving the compound of the invention in a suitably flavored aqueous solution, while elixirs are prepared through the use of a non-toxic alcoholic vehicle. Suspensions can be formulated by dispersing the compound of the invention in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

In another aspect, the invention is directed to parenteral administration. Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Compounds or pharmaceutically acceptable salts or tautomer forms thereof of the present invention or corresponding pharmaceutical compositions thereof as defined throughout the present application may be administered parenterally or orally as an injecting agent, capsules, tablets, and granules, and preferably, administered as an injecting agent.

Carriers when used as an injecting agent is for example, distilled water, saline and the like, and base and the like may be used for pH adjustment.

When used as capsules, granules or tablets, carriers may be known excipients (e.g., starch, lactose, sucrose, calcium carbonate, calcium phosphate and the like), binders (e.g., starch, acacia gum, carboxymethyl cellulose, hydroxypropyl cellulose, crystalline cellulose, and the like), lubricants (e.g., magnesium stearate, talc and the like), and the like.

It will also be recognized by one of skill in the art that the optimal quantity and spacing of individual dosages of compounds or pharmaceutically acceptable salts or tautomer forms thereof of the present invention or corresponding pharmaceutical compositions thereof as defined throughout the present application will be determined by the nature and extent of the condition being treated, the form, route and site of administration, and the particular patient being treated, and that such optimums can be determined by conventional techniques.

It will also be appreciated by one of skill in the art that the optimal course of treatment, i.e., the number of doses of a compound of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) of the present invention given per day for a defined number of days, can be ascertained by those skilled in the art using conventional course of treatment determination tests.

The amount of compounds or pharmaceutically acceptable salts or tautomer forms thereof of the present invention or corresponding pharmaceutical compositions thereof as defined throughout the present application which is required to achieve a therapeutic effect will, of course, vary with the particular compound, the route of administration, the subject under treatment, and the particular disorder or disease being treated.

Suitable dosing regimens for a compound of the invention depend on the pharmacokinetic properties of that compound, such as absorption, distribution, and half-life, which can be determined by the skilled artisan.

In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Additionally, the compounds of the present invention generally may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following:

(a) modify the onset of the compound in vivo;

(b) modify the duration of action of the compound in vivo;

(c) modify the transportation or distribution of the compound in vivo;

(d) modify the solubility of the compound in vivo; and

(e) overcome a side effect or other difficulty encountered with the compound.

Typical functional derivatives used to prepare prodrugs include modifications of the compound that are chemically or enzymatically cleaved in vivo. Such modifications, which include the preparation of phosphates, amides, esters, thioesters, carbonates, and carbamates, are well known to those skilled in the art.

The invention also provides a compound of the invention for use in medical therapy, particularly in those diseases as defined throughout the instant application, such as:
pain-associated disease(s), disorder(s) or condition(s), such as pain caused by a variety of diseases;
pain caused by trauma; or
pain caused by iatrogenic (i.e., such as medical or dental) procedures, etc.

Thus, in a further aspect, the invention is directed to the use of a compound according to Formula I or a pharmaceutically-acceptable salt thereof in the preparation of a medicament for the treatment of in the aforementioned diseases defined above and as defined throughout the instant application.

Doses of the presently invented pharmaceutically active compounds in a pharmaceutical dosage unit as described above will be an efficacious, nontoxic quantity preferably selected from the range of 0.001-100 mg/kg of active compound, preferably 0.001-50 mg/kg. When treating a human patient in need of a Nav1.8 inhibition, the selected dose is administered preferably from 1-6 times daily, orally or parenterally.

Preferred forms of parenteral administration include topically, rectally, transdermally, by injection and continuously by infusion. Oral dosage units for human administration preferably contain from 0.05 to 3500 mg of active compound. Oral administration, which uses lower dosages, is preferred. Parenteral administration, at high dosages, however, also can be used when safe and convenient for the patient.

The invention also provides a pharmaceutical composition comprising from 0.5 to 1,000 mg of a compound of Formula (X) or pharmaceutically acceptable salt thereof and from 0.5 to 1,000 mg of a pharmaceutically acceptable excipient.

### Combination Therapies and Uses Thereof for Therapy

In general, the invention relates to combination therapies, methods, compounds for use in or uses, in which a patient or subject in need thereof is treated with one or more additional therapeutic agents administered concurrently with, prior to, or subsequent to treatment with an effective amount of a compound of any of the Formulas disclosed herein, including Formula (X) and Formulas (I) to (III) (i.e., inc. corresponding subgeneric formulas defined herein), respectively, or a pharmaceutically acceptable salt and/or a corresponding tautomer form thereof (i.e., including subgeneric formulas, as defined above) of the present invention or a corresponding pharmaceutical composition thereof.

Active drug or therapeutic agents, when employed in combination with the compounds, or pharmaceutical compositions of the present invention, may be used or administered, for example, in dosage amounts indicated in the Physicians' Desk Reference (PDR) or as otherwise determined by one of ordinary skill in the art.

In the context of this specification, the term “simultaneously” when referring to simultaneous administration of the relevant drugs means at exactly the same time, as would be the case, for example in embodiments where the drugs are combined in a single preparation. In other aspects or embodiments, “simultaneously” can mean one drug taken a short duration after another, wherein “a short duration” means a duration which allows the drugs to have their intended synergistic effect.

In light of the foregoing, the present invention also relates to a combination therapy, which may be a comprised of a simultaneous or co-administration, or serial administration of a combination of compounds or pharmaceutical compositions of the present invention with other active drug or therapeutic agents, such as which include, but are not limited to: Acetaminophen, Acetylsalicylic acid, Nav1.7 Inhibitors, Nav1.9 Inhibitors, anti-depressants (i.e. such as, but not limited to duloxetine or amitriptyline), anti-convulsants (i.e. such as, but not limited to pregabalin and gabapentin), opiates (i.e., such as, but not limited to hydrocodone; codeine; morphine, oxycodone, oxymorphone, fentanyl, and the like), etc.; and where administration of the above, respectively, also is determined by one of ordinary skill in the art. In one aspect, suitable Nav1.7 Inhibitors or Nav1.9 Inhibitors for use in the present invention, include, but are not limited to those Nav1.7 Inhibitors or Nav1.9 Inhibitors known in the chemical literature.

Another example of a combination therapy of the present invention involves combining subtherapeutic doses of acetaminophen or acetylsalicylic acid with subtherapeutic doses of an oral Nav1.8 inhibitor, such as the compounds of the present invention described herein, so the synergistic actions of these agents provides adequate pain relief but reduces the side effect profile and risks associated with using therapeutic dose of these agents as monotherapy.

In another example of a combination therapy of the present invention involves combining subtherapeutic doses of an oral opioid receptor antagonist with subtherapeutic doses of an oral Nav1.8 inhibitor so the synergistic actions of these agents provide adequate pain relief, but reduces the side effect profile and risks associated with using therapeutic dose of these agents as monotherapy.

In yet another aspect, an example of a combination therapy of the present invention involves initial treatment with an intravenous or parenteral Naᵥ1.8 inhibitor formulation to achieve rapid pain relief, which is followed by treatment with an oral Naᵥ1.8 inhibitor formulation to maintain longer term pain relief.

In another aspect, the present invention relates to a combination therapy for treating:
pain-associated disease(s), disorder(s) or condition(s);
pain caused by trauma; or
pain caused by iatrogenic medical or dental procedure(s);
   which comprises simultaneous administration, co-administration, or serial administration of a therapeutically effective combination of component(s):
[a] compound of any of the Formulas disclosed herein, including Formula (X), including Formula (I), or a pharmaceutically acceptable salt or a corresponding tautomer form thereof; or
[b] a corresponding pharmaceutical composition or formulation thereof; and
[c] other active drug or therapeutic agents selected from:
   Acetaminophen;
   Acetylsalicylic acid;
   Naᵥ 1.7 Inhibitors;
   Naᵥ 1.9 Inhibitors;
   Anti-depressants
   Anti-convulsants or
   Opiates;
      to a patient or subject in need thereof.

In another aspect, the present invention relates to a combination therapy, where each component of such a combination used for therapeutic purposes may be administered orally, intravenously or parenterally or in combinations thereof.

Other aspects, also indicate that each component of an aforementioned combination may be administered in subtherapeutic doses.

In another aspect, the present invention relates to a combination therapy, where:
each component of a therapeutic combination may be, but is not limited to being:
   administered by simultaneous administration, co-administration, or serial administration; and/or
   by identical or different routes of administration or combinations of administration routes;
   where:
      each identical or different route of administration or combinations of administration routes is selected from oral, intravenous or parenteral administration.

In another aspect, the present invention relates to a combination therapy, which uses opiates which are selected from, but not limited to hydrocodone; codeine; morphine, oxycodone, oxymorphone or fentanyl and the like.

In another aspect, the present invention relates to a combination therapy, which uses anti-depressants are selected from, but not limited to duloxetine or amitriptyline and the like.

In another aspect, the present invention relates to a combination therapy, which uses anti-convulsants are selected from, but not limited to pregabalin and gabapentin and the like.

In another aspect, each active or therapeutic agent(s) as defined herein, is administered in subtherapeutic doses. For example, active or therapeutic agents, such as, but not limited to, acetaminophen or acetylsalicylic acid, respectively may be administered in subtherapeutic doses.

In another aspect, the present invention relates to a combination therapy for purposes as define above and throughout the instant specification, which comprises simultaneously administering or co-administrating, or serial administration of a therapeutically effective combination of:
[a] a subtherapeutic dose(s) of an oral Naᵥ 1.8 inhibitor agent of the present invention, i.e., a compound of any of the Formulas disclosed herein, including Formula (I) or a pharmaceutically acceptable salt or a corresponding tautomer form thereof; or a corresponding pharmaceutical composition thereof;
[b] subtherapeutic doses of an oral opioid receptor antagonist agent;
   to a patient or subject in need thereof.

In another aspect of the present invention, synergistic actions of the combination of:
[a] the subtherapeutic dose(s) of a Naᵥ 1.8 inhibitor agent or compound of the present invention or as defined herein; and
[b] the subtherapeutic dose(s) of the opioid receptor antagonist agent or compound of the present invention or as defined herein provides pain relief and reduces the side effects and risks associated with using therapeutic dose(s) of each of above agents individually or separately as monotherapy or monotherapies, respectively.

In another aspect, the present invention relates to a combination therapy for purposes as define above and throughout the instant specification, which comprises serial administration of a therapeutically effective combination of:
[a] initial treatment to achieve rapid pain relief with an intravenous or parenteral administration of a Naᵥ 1.8 inhibitor compound or a pharmaceutically acceptable salt a corresponding tautomer form thereof; or a corresponding pharmaceutical composition or formulation of the present invention or as known in the art; and
[b] followed by treatment to maintain longer term pain relief with an identical or different oral Nav1.8 inhibitor compound or pharmaceutically acceptable salt and/or tautomer form thereof or a corresponding pharmaceutical composition or formulation of the present invention or as known in the art.

In another aspect, the present invention relates to a combination therapy for purposes as define above and throughout the instant specification, which comprises serial administration of a therapeutically effective combination of:
[a] initial treatment to achieve rapid pain relief with an intravenous or parenteral administration of:
   compound of any of the Formulas disclosed herein, including Formula (I) or a pharmaceutically acceptable salt or a corresponding tautomer form thereof of the present invention; and/or
   a pharmaceutical composition or formulation of the present invention; and
[b] followed by treatment to maintain longer term pain relief with an oral administration of:
   compound of any of the Formulas disclosed herein, including Formula (I) or a pharmaceutically acceptable salt or a corresponding tautomer form thereof; and/or
   a pharmaceutical composition or formulation of the present invention.

In another aspect, the present invention relates to a combination therapy for purposes as define above and throughout the instant specification, which comprises serial administration of a therapeutically effective combination of:
[a] initial treatment to achieve rapid pain relief with an intravenous or parenteral administration of a pharmaceutical composition or formulation of the present invention; and
[b] followed by treatment to maintain longer term pain relief with an oral administration of a pharmaceutical composition or formulation of the present invention.

In another aspect, the present invention relates to:
a compound of any of the Formulas disclosed herein, including Formula (I) or a pharmaceutically acceptable salt and/or a corresponding tautomer form thereof; or
a corresponding pharmaceutical composition thereof

for use in therapy.

In another aspect, the present invention relates to:
compound of any of the Formulas disclosed herein, including Formula (I) or a pharmaceutically acceptable salt and/or a corresponding tautomer form thereof; or
a corresponding pharmaceutical composition thereof

for use in combination therapy for treating:
pain-associated disease(s), disorder(s) or condition(s);
pain caused by trauma; or
pain caused by iatrogenic medical or dental procedure(s);
   to a patient or subject in need thereof.

In yet another aspect, the present invention also relates a combination therapy for the as described herein, which is comprised of a composition, dosage form or formulation formed from a synergistic combination or mixture of compounds of the present invention, corresponding controlled release compositions, dosage forms or formulations, which may include another active drug or therapeutic agent or agents as those described herein and optionally which comprises pharmaceutically acceptable carrier, diluent or adjuvant.

Moreover, in such an aforementioned combination composition, dosage form or formulation, each of the active drug components are contained in therapeutically effective and synergistic dosage amounts.

The Examples set forth below are illustrative of the present invention and are not intended to limit, in any way, the scope of the present invention.

### EXAMPLES

The following examples illustrate the invention. These examples are not intended to limit the scope of the present invention, but rather to provide guidance to the skilled artisan to prepare and use the compounds, compositions, and methods of the present invention.

While particular aspects or embodiments of the present invention are described, the skilled artisan will appreciate that various changes and modifications can be made without departing from the spirit and scope of the invention.

It will be understood by the skilled artisan that purification methods (using acidic or basic modifiers) or compound workup procedures (using acidic or basic conditions) may result in formation of a salt of a title compound (for example, hydrobromic acid, formic acid, hydrochloric acid, trifluoroacetic acid, or ammonia salts of a title compound). The present invention is intended to encompass such salts.

### Biology and Biological Assays

The Naᵥ1.8 Inhibitor 2,3-dihydroquinazolin-4(1H)-one compounds or pharmaceutically acceptable salts thereof, are useful for treatment of pain, pain disorders or conditions, pain-related disorders or conditions or pain caused by diseases, respectively, such as those defined throughout the instant application.

The biological activity of the compounds of the present invention can be determined using suitable assays, such as those measuring such inhibition and those evaluating the ability of the compounds to inhibit voltage gated sodium channel Naᵥ 1.8 in vitro or in animal models of infection.

### Biological Assay Example 1

Human embryonic kidney 2993 cells (HEK293) expressing human Naᵥ1.8, human Naᵥβ1 and human TREK1 (HEK293-Naᵥ1.8) were grown at 37° C., 5% CO₂ in 150 cm² flasks. HEK293-Naᵥ1.8 were passaged every 2-3 days when confluency reached 80-90% in to T175 cell culture flasks.

Pharmacological assessment of the invention was performed using HEK293-Nav1.8 in combination with an assay developed on the QPatch 48 HTX electrophysiological system. HEK293-Naᵥ1.8 were prepared on the day of use by removing culture media, washing in DPBS, adding Accutase (2 ml to cover the surface, aspirate 1 ml then 1.5 min at 37° C.) followed by addition of CHO-SFM II to stop the enzyme digestion and in order to obtain a suspension of 3×10⁶ cell/mL.

The invention was prepared in an extracellular solution of the following composition (in mM) NaCl 145, KCl 4, CaCl₂ 2, MgCl 2, HEPES 1, Glucose 10, pH 7.4 with NaOH Osmolality 300 mOsM/L. The intracellular solution was used of the following composition (in mM) CsF 115, CsCl 20, NaCl 5, EGTA 10, HEPES 10, Sucrose 20, pH 7.2 with CsOH Osmolality 310 mOsm/L.

Utilizing the voltage-clamp mode in the QPatch 48 HTX system a half inactivation state voltage protocol (V_{1/2}) was used to determine pharmacological activity of the invention at Naᵥ1.8 ion channels. A V_{1/2} protocol was utilized with the following voltage steps: a holding voltage of −100 mV was established followed by a 20 ms voltage step to 0 mV (P1), followed by an inactivating voltage step at −46 mV for 8 seconds, followed by a step to −100 mV for 20 ms, before a 20 ms step to 0 mV (P2) before returning to the holding voltage of −100 mV. This voltage protocol was repeated at a frequency of 0.07 Hz., current magnitude was quantified at the P2 step throughout the recording. Inhibition of the measured current amplitude with the invention was analyzed by fitting a 6-8 point dose-response curve allowing determination of the fifty percent inhibition concentration (IC₅₀). Within the QPatch HTX software, P2 current was normalized according to measurements made at baseline after compound and after positive reference compound and fit to the following equation:

$$n . {I}_{C P D} = Normalized Current = \frac{( Input - Baseline )}{( FullResponse - Baseline )}$$

To assess current run-down over the course of the experiment vehicle-only wells were utilized and the normalized current with vehicle-only (n.I_{VEH}) was determined. To correct the compound response for run-down, the currents were correct according the following formula:

$$n . {I}_{RD\_Correct} = \frac{( n . {I}_{CPD} - n . {I}_{V E H} )}{( 1 - n . {I}_{V E H} )}$$

Compounds of the invention are tested for activity against Nav1. 8 sodium channels in the above assay.

The compounds of the Examples were tested, in at least one exemplified salt or free base form, generally according to the above Nav1. 8 sodium channels assay and in at least one experimental run exhibited a pIC50 value, or in a set of two or more experimental runs exhibited an average pIC50 value, of: 5.1 against Nav1. 8 sodium channels.

The compounds of Examples 1, 2, 3, 5, 6, 7, 8, 9, 10, 13, 14, 15, 17, 18, 19, 20, 22, 24 to 27, 29 to 32, 37, 38, 39, 41, 42, 44, 45, 46, 49, 53, 54, 55, 59, 62, 80, 84, 86, 94, 100, 124, 125, 133, 135, 136, 151, 163, 186, 195, 197, 202, 204, 208, 211, 217, 219, 220, 222, 223, 224, 229, 232, 235, 236, 237, 238, 248, 249, 250 to 253, 255, 258 to 264, 266, 267, 272 to 276, 282, 284, 285, 286, 287, 289, 291 and 295 were tested generally according to the above Nav1. 8 sodium channels assay and in at least one set of experimental runs exhibited an average pIC50 value: ≥5.1 and ≤6.1 against Nav1.8.

The compounds of Examples 4, 11, 12, 16, 21, 23, 28, 34, 36, 40, 43, 50, 58, 67, 68, 71, 75, 77, 78, 82, 88, 92, 102, 113, 118, 119, 121, 127, 128, 130, 132, 134, 140, 141, 144, 145, 146, 147, 149, 154, 155, 156, 157, 164, 165, 169, 174, 175, 179, 181, 183, 196, 199, 206, 209, 210, 213, 215, 216, 218, 221, 239, 241, 243, 254, 265, 268, 270, 277, 280, 281, 283, 288, 290, 292, 293 and 299 were tested generally according to the above Nav1. 8 sodium channels assay and in at least one set of experimental runs exhibited an average pIC50 value: 6.2 and ≤6.9 against NAV1.8.

The compounds of Examples 33, 35, 47, 48, 51, 52, 56, 57, 60, 61, 63, 64, 65, 66, 69, 70, 72, 73, 74, 76, 79, 81, 83, 85, 87, 89, 90, 91, 93, 95, 96, 97, 98, 99, 101, 103, 104, 105, 106, 107, 108, 109, 110, 111, 112, 114, 115, 116, 117, 120, 122, 123, 126, 129, 131, 137, 138, 139, 142, 143, 148, 150, 152, 153, 158, 159, 160, 161, 162, 166, 167, 168, 170, 171, 172, 173, 176, 177, 178, 180, 182, 184, 185, 187, 188, 189, 190, 191, 192, 193, 194, 198, 200, 201, 203, 205, 207, 212, 214, 225, 226, 227, 230, 231, 233, 234, 240, 242, 244, 245, 246, 247, 256, 257, 269, 271, 278, 279, 294, 296, 297 and 298 were tested generally according to the above Nav1. 8 sodium channels assay and in at least one set of experimental runs exhibited an average pIC50 value: 7.0 against Nav1.8.

Compounds of the invention were tested, in at least one exemplified salt or free base form, generally according to the above Nav1. 8 sodium channels assay and in at least one experimental run exhibited a pIC50 value, or in a set of two or more experimental runs exhibited an average pIC50 value indicated in Table 1 and Table 2 below.

TABLE 1

| | Naᵥ1.8 | |
|---|---|---|
| Structure | pIC₅₀ | Name |
| 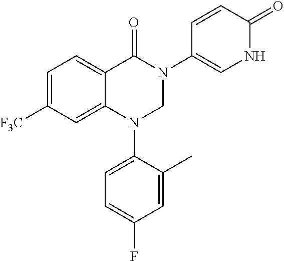 [SMILES: Cc1cc(F)ccc1N1CN(c2ccc(=O)[nH]c2)C(=O)c2ccc(C(F)(F)F)cc21] | 6.9 | 1-(4-fluoro-2-methylphenyl)-3- (6-oxo-1,6-dihydropyridin-3-yl)- 7-(trifluoromethyl)-2,3- dihydroquinazolin-4(1H)-one |
| 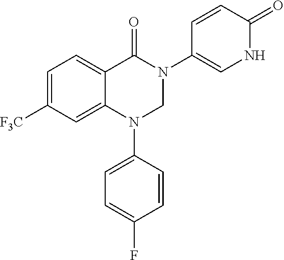 [SMILES: O=C1c2ccc(C(F)(F)F)cc2N(c2ccc(F)cc2)CN1c1ccc(=O)[nH]c1] | 6.4 | 1-(4-fluorophenyl)-3-(6-oxo-1,6- dihydropyridin-3-yl)-7- (trifluoromethyl)-2,3- dihydroquinazolin-4(1H)-one |
| 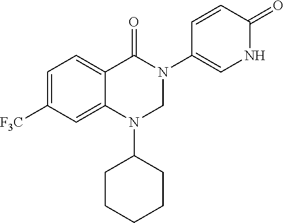 [SMILES: O=C1c2ccc(C(F)(F)F)cc2N(C2CCCCC2)CN1c1ccc(=O)[nH]c1] | 5.2 | 1-cyclohexyl-3-(6-oxo-1,6- dihydropyridin-3-yl)-7- (trifluoromethyl)-2,3- dihydroquinazolin-4(1H)-one |
| 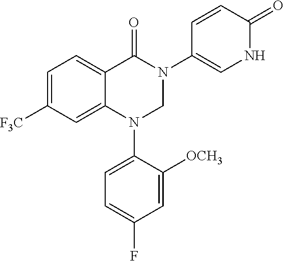 [SMILES: COc1cc(F)ccc1N1CN(c2ccc(=O)[nH]c2)C(=O)c2ccc(C(F)(F)F)cc21] | 6.5 | 1-(4-fluoro-2-methoxyphenyl)-3- (6-oxo-1,6-dihydropyridin-3-yl)- 7-(trifluoromethyl)-2,3- dihydroquinazolin-4(1H)-one |
| 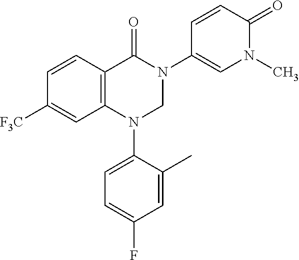 [SMILES: Cc1cc(F)ccc1N1CN(c2ccc(=O)n(C)c2)C(=O)c2ccc(C(F)(F)F)cc21] | 5.7 | 1-(4-fluoro-2-methylphenyl)-3- (1-methyl-6-oxo-1,6- dihydropyridin-3-yl)-7- (trifluoromethyl)-2,3- dihydroquinazolin-4(1H)-one |
| 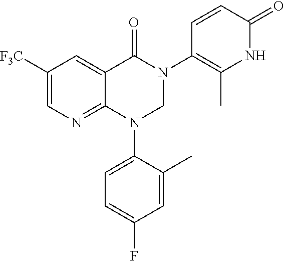 [SMILES: Cc1cc(F)ccc1N1CN(c2ccc(=O)[nH]c2C)C(=O)c2cc(C(F)(F)F)cnc21] | 6.8 | 1-(4-fluoro-2-methylphenyl)-3- (2-methyl-6-oxo-1,6- dihydropyridin-3-yl)-6- (trifluoromethyl)-2,3- dihydropyrido[2,3-d]pyrimidin- 4(1H)-one |
| 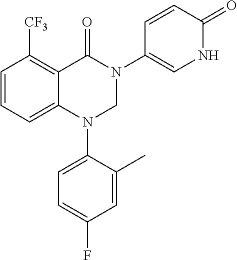 [SMILES: Cc1cc(F)ccc1N1CN(c2ccc(=O)[nH]c2)C(=O)c2c1cccc2C(F)(F)F] | 5.6 | 1-(4-fluoro-2-methylphenyl)-3- (6-oxo-1,6-dihydropyridin-3-yl)- 5-(trifluoromethyl)-2,3- dihydroquinazolin-4(1H)-one |
| 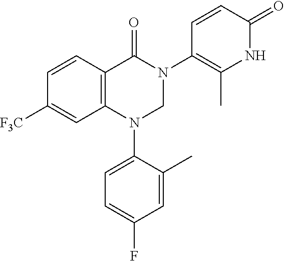 [SMILES: Cc1cc(F)ccc1N1CN(c2ccc(=O)[nH]c2C)C(=O)c2ccc(C(F)(F)F)cc21] | 7.7 | 1-(4-fluoro-2-methylphenyl)-3- (2-methyl-6-oxo-1,6- dihydropyridin-3-yl)-7- (trifluoromethyl)-2,3- dihydroquinazolin-4(1H)-one |
| 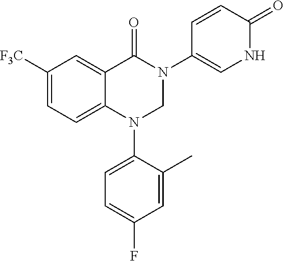 [SMILES: Cc1cc(F)ccc1N1CN(c2ccc(=O)[nH]c2)C(=O)c2cc(C(F)(F)F)ccc21] | 6.5 | 1-(4-fluoro-2-methylphenyl)-3- (6-oxo-1,6-dihydropyridin-3-yl)- 6-(trifluoromethyl)-2,3- dihydroquinazolin-4(1H)-one |
| 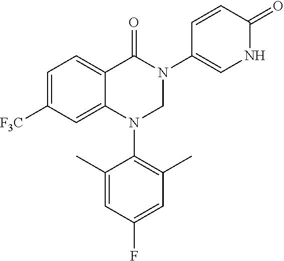 [SMILES: Cc1cc(F)cc(C)c1N1CN(c2ccc(=O)[nH]c2)C(=O)c2ccc(C(F)(F)F)cc21] | 5.6 | 1-(4-fluoro-2,6-dimethylphenyl)- 3-(6-oxo-1,6-dihydropyridin-3- yl)-7-(trifluoromethyl)-2,3- dihydroquinazolin-4(1H)-one |
| 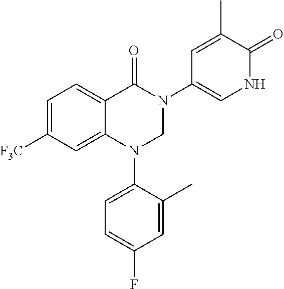 [SMILES: Cc1cc(F)ccc1N1CN(c2c[nH]c(=O)c(C)c2)C(=O)c2ccc(C(F)(F)F)cc21] | 6.2 | 1-(4-fluoro-2-methylphenyl)-3- (5-methyl-6-oxo-1,6- dihydropyridin-3-yl)-7- (trifluoromethyl)-2,3- dihydroquinazolin-4(1H)-one |
| 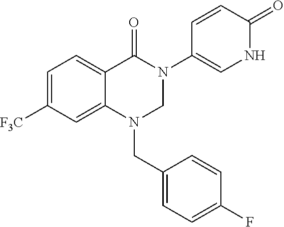 [SMILES: O=C1c2ccc(C(F)(F)F)cc2N(Cc2ccc(F)cc2)CN1c1ccc(=O)[nH]c1] | 5.1 | 1-(4-fluorobenzyl)-3-(6-oxo-1,6- dihydropyridin-3-yl)-7- (trifluoromethyl)-2,3- dihydroquinazolin-4(1H)-one |
| 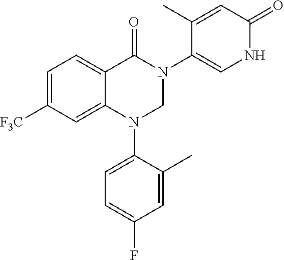 [SMILES: Cc1cc(=O)[nH]cc1N1CN(c2ccc(F)cc2C)c2cc(C(F)(F)F)ccc2C1=O] | 6.4 | 1-(4-fluoro-2-methylphenyl)-3- (4-methyl-6-oxo-1,6- dihydropyridin-3-yl)-7- (trifluoromethyl)-2,3- dihydroquinazolin-4(1H)-one |
| 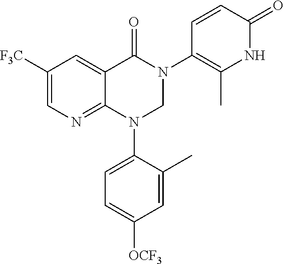 [SMILES: Cc1cc(OC(F)(F)F)ccc1N1CN(c2ccc(=O)[nH]c2C)C(=O)c2cc(C(F)(F)F)cnc21] | 7.6 | 1-(2-methyl-4- (trifluoromethoxy)phenyl)-3-(2- methyl-6-oxo-1,6- dihydropyridin-3-yl)-6- (trifluoromethyl)-2,3- dihydropyrido[2,3-d]pyrimidin- 4(1H)-one |
| 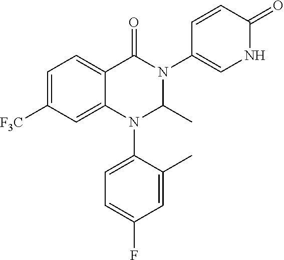 [SMILES: Cc1cc(F)ccc1N1c2cc(C(F)(F)F)ccc2C(=O)N(c2ccc(=O)[nH]c2)C1C] | 5.9 | 1-(4-fluoro-2-methylphenyl)-2- methyl-3-(6-oxo-1,6- dihydropyridin-3-yl)-7- (trifluoromethyl)-2,3- dihydroquinazolin-4(1H)-one |
| 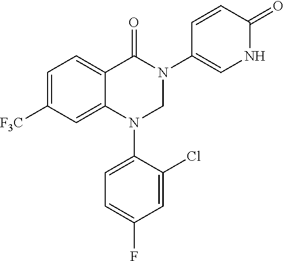 [SMILES: O=C1c2ccc(C(F)(F)F)cc2N(c2ccc(F)cc2Cl)CN1c1ccc(=O)[nH]c1] | 6.3 | 1-(2-chloro-4-fluorophenyl)-3- (6-oxo-1,6-dihydropyridin-3-yl)- 7-(trifluoromethyl)-2,3- dihydroquinazolin-4(1H)-one |
| 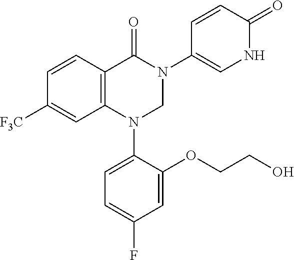 [SMILES: O=C1c2ccc(C(F)(F)F)cc2N(c2ccc(F)cc2OCCO)CN1c1ccc(=O)[nH]c1] | 5.2 | 1-(4-fluoro-2-(2- hydroxyethoxy)phenyl)-3-(6- oxo-1,6-dihydropyridin-3-yl)-7- (trifluoromethyl)-2,3- dihydroquinazolin-4(1H)-one |
| 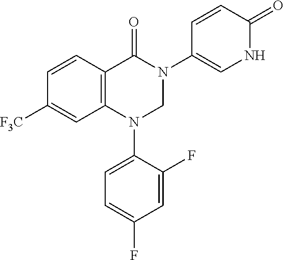 [SMILES: O=C1c2ccc(C(F)(F)F)cc2N(c2ccc(F)cc2F)CN1c1ccc(=O)[nH]c1] | 6.1 | 1-(2,4-difluorophenyl)-3-(6-oxo- 1,6-dihydropyridin-3-yl)-7- (trifluoromethyl)-2,3- dihydroquinazolin-4(1H)-one |
| 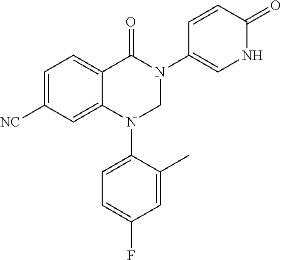 [SMILES: Cc1cc(F)ccc1N1CN(c2ccc(=O)[nH]c2)C(=O)c2ccc(C#N)cc21] | 5.8 | 1-(4-fluoro-2-methylphenyl)-4- oxo-3-(6-oxo-1,6- dihydropyridin-3-yl)-1,2,3,4- tetrahydroquinazoline-7- carbonitrile |
| 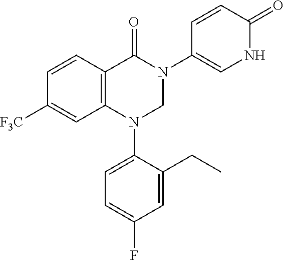 [SMILES: CCc1cc(F)ccc1N1CN(c2ccc(=O)[nH]c2)C(=O)c2ccc(C(F)(F)F)cc21] | 6.6 | 1-(2-ethyl-4-fluorophenyl)-3-(6- oxo-1,6-dihydropyridin-3-yl)-7- (trifluoromethyl)-2,3- dihydroquinazolin-4(1H)-one |
| 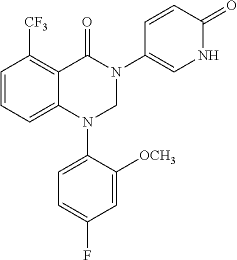 [SMILES: COc1cc(F)ccc1N1CN(c2ccc(=O)[nH]c2)C(=O)c2c1cccc2C(F)(F)F] | 5.2 | 1-(4-fluoro-2-methoxyphenyl)-3- (6-oxo-1,6-dihydropyridin-3-yl)- 5-(trifluoromethyl)-2,3- dihydroquinazolin-4(1H)-one |
| 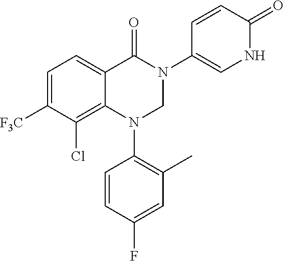 [SMILES: Cc1cc(F)ccc1N1CN(c2ccc(=O)[nH]c2)C(=O)c2ccc(C(F)(F)F)c(Cl)c21] | 5.2 | 8-chloro-1-(4-fluoro-2- methylphenyl)-3-(6-oxo-1,6- dihydropyridin-3-yl)-7- (trifluoromethyl)-2,3- dihydroquinazolin-4(1H)-one |
| 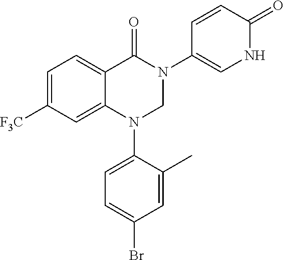 [SMILES: Cc1cc(Br)ccc1N1CN(c2ccc(=O)[nH]c2)C(=O)c2ccc(C(F)(F)F)cc21] | 6.5 | 1-(4-bromo-2-methylphenyl)-3- (6-oxo-1,6-dihydropyridin-3-yl)- 7-(trifluoromethyl)-2,3- dihydroquinazolin-4(1H)-one |
| 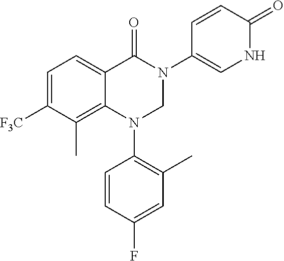 [SMILES: Cc1cc(F)ccc1N1CN(c2ccc(=O)[nH]c2)C(=O)c2ccc(C(F)(F)F)c(C)c21] | 5.2 | 1-(4-fluoro-2-methylphenyl)-8- methyl-3-(6-oxo-1,6- dihydropyridin-3-yl)-7- (trifluoromethyl)-2,3- dihydroquinazolin-4(1H)-one |
| 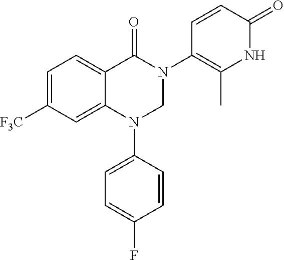 [SMILES: Cc1[nH]c(=O)ccc1N1CN(c2ccc(F)cc2)c2cc(C(F)(F)F)ccc2C1=O] | 6.9 | 1-(4-fluorophenyl)-3-(2-methyl- 6-oxo-1,6-dihydropyridin-3-yl)- 7-(trifluoromethyl)-2,3- dihydroquinazolin-4(1H)-one |
| 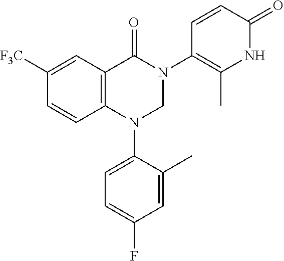 [SMILES: Cc1cc(F)ccc1N1CN(c2ccc(=O)[nH]c2C)C(=O)c2cc(C(F)(F)F)ccc21] | 7.8 | 1-(4-fluoro-2-methylphenyl)-3- (2-methyl-6-oxo-1,6- dihydropyridin-3-yl)-6- (trifluoromethyl)-2,3- dihydroquinazolin-4(1H)-one |
| 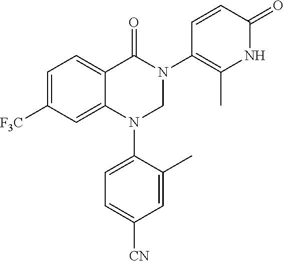 [SMILES: Cc1cc(C#N)ccc1N1CN(c2ccc(=O)[nH]c2C)C(=O)c2ccc(C(F)(F)F)cc21] | 7.2 | 3-methyl-4-(3-(2-methyl-6-oxo- 1,6-dihydropyridin-3-yl)-4-oxo- 7-(trifluoromethyl)-3,4- dihydroquinazolin-1(2H)- yl)benzonitrile |
| 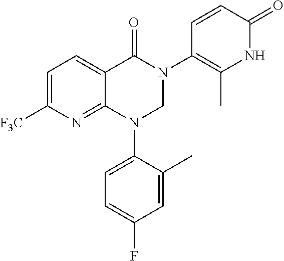 [SMILES: Cc1cc(F)ccc1N1CN(c2ccc(=O)[nH]c2C)C(=O)c2ccc(C(F)(F)F)nc21] | 6.6 | 1-(4-fluoro-2-methylphenyl)-3- (2-methyl-6-oxo-1,6- dihydropyridin-3-yl)-7- (trifluoromethyl)-2,3- dihydropyrido[2,3-d]pyrimidin- 4(1H)-one |
| 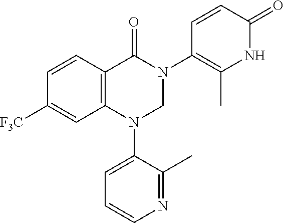 [SMILES: Cc1ncccc1N1CN(c2ccc(=O)[nH]c2C)C(=O)c2ccc(C(F)(F)F)cc21] | 5.6 | 3-(2-methyl-6-oxo-1,6- dihydropyridin-3-yl)-1-(2- methylpyridin-3-yl)-7- (trifluoromethyl)-2,3- dihydroquinazolin-4(1H)-one |
| 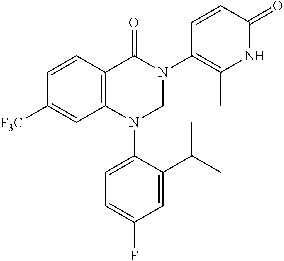 [SMILES: Cc1[nH]c(=O)ccc1N1CN(c2ccc(F)cc2C(C)C)c2cc(C(F)(F)F)ccc2C1=O] | 8.2 | 1-(4-fluoro-2-isopropylphenyl)- 3-(2-methyl-6-oxo-1,6- dihydropyridin-3-yl)-7- (trifluoromethyl)-2,3- dihydroquinazolin-4(1H)-one |
| 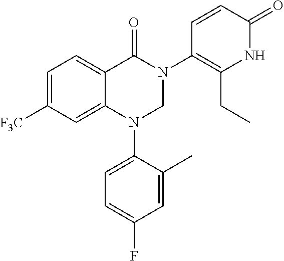 [SMILES: CCc1[nH]c(=O)ccc1N1CN(c2ccc(F)cc2C)c2cc(C(F)(F)F)ccc2C1=O] | 7.7 | 3-(2-ethyl-6-oxo-1,6- dihydropyridin-3-yl)-1-(4-fluoro- 2-methylphenyl)-7- (trifluoromethyl)-2,3- dihydroquinazolin-4(1H)-one |
| 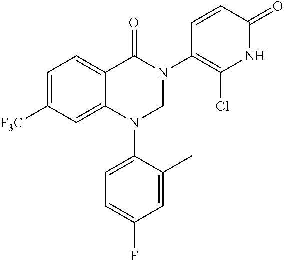 [SMILES: Cc1cc(F)ccc1N1CN(c2ccc(=O)[nH]c2Cl)C(=O)c2ccc(C(F)(F)F)cc21] | 6.1 | 3-(2-chloro-6-oxo-1,6- dihydropyridin-3-yl)-1-(4-fluoro- 2-methylphenyl)-7- (trifluoromethyl)-2,3- dihydroquinazolin-4(1H)-one |
| 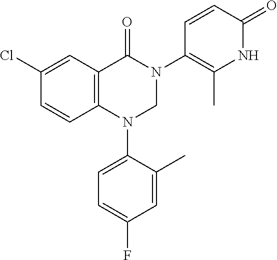 [SMILES: Cc1cc(F)ccc1N1CN(c2ccc(=O)[nH]c2C)C(=O)c2cc(Cl)ccc21] | 8.0 | 6-chloro-1-(4-fluoro-2- methylphenyl)-3-(2-methyl-6- oxo-1,6-dihydropyridin-3-yl)-2,3- dihydroquinazolin-4(1H)-one |
| 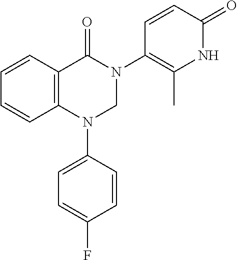 [SMILES: Cc1[nH]c(=O)ccc1N1CN(c2ccc(F)cc2)c2ccccc2C1=O] | 5.3 | 1-(4-fluorophenyl)-3-(2-methyl- 6-oxo-1,6-dihydropyridin-3-yl)- 2,3-dihydroquinazolin-4(1H)- one |
| 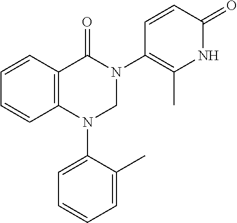 [SMILES: Cc1ccccc1N1CN(c2ccc(=O)[nH]c2C)C(=O)c2ccccc21] | 6.2 | 3-(2-methyl-6-oxo-1,6- dihydropyridin-3-yl)-1-(o-tolyl)- 2,3-dihydroquinazolin-4(1H)- one |
| 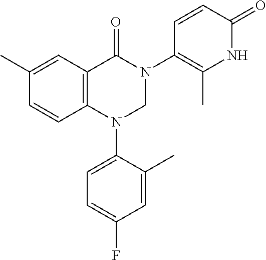 [SMILES: Cc1ccc2c(c1)C(=O)N(c1ccc(=O)[nH]c1C)CN2c1ccc(F)cc1C] | 7.3 | 1-(4-fluoro-2-methylphenyl)-6- methyl-3-(2-methyl-6-oxo-1,6- dihydropyridin-3-yl)-2,3- dihydroquinazolin-4(1H)-one |
| 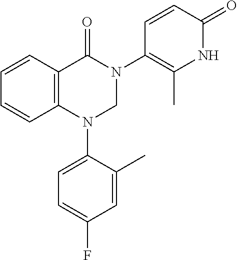 [SMILES: Cc1cc(F)ccc1N1CN(c2ccc(=O)[nH]c2C)C(=O)c2ccccc21] | 7.2 | 1-(4-fluoro-2-methylphenyl)-3- (2-methyl-6-oxo-1,6- dihydropyridin-3-yl)-2,3- dihydroquinazolin-4(1H)-one |
| 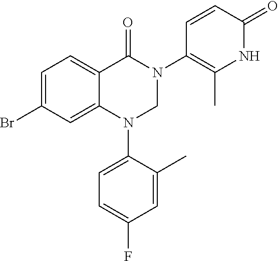 [SMILES: Cc1cc(F)ccc1N1CN(c2ccc(=O)[nH]c2C)C(=O)c2ccc(Br)cc21] | 8.5 | 7-bromo-1-(4-fluoro-2- methylphenyl)-3-(2-methyl-6- oxo-1,6-dihydropyridin-3-yl)-2,3- dihydroquinazolin-4(1H)-one |
| 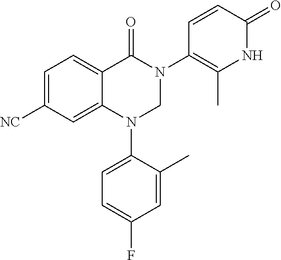 [SMILES: Cc1cc(F)ccc1N1CN(c2ccc(=O)[nH]c2C)C(=O)c2ccc(C#N)cc21] | 7.6 | 1-(4-fluoro-2-methylphenyl)-3- (2-methyl-6-oxo-1,6- dihydropyridin-3-yl)-4-oxo- 1,2,3,4-tetrahydroquinazoline-7- carbonitrile |
| 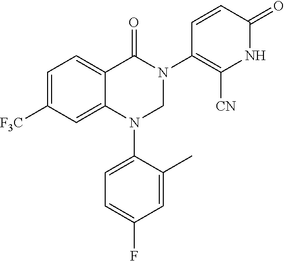 [SMILES: Cc1cc(F)ccc1N1CN(c2ccc(=O)[nH]c2C#N)C(=O)c2ccc(C(F)(F)F)cc21] | 6.6 | 3-(1-(4-fluoro-2-methylphenyl)- 4-oxo-7-(trifluoromethyl)-1,4- dihydroquinazolin-3(2H)-yl)-6- oxo-1,6-dihydropyridine-2- carbonitrile |
| 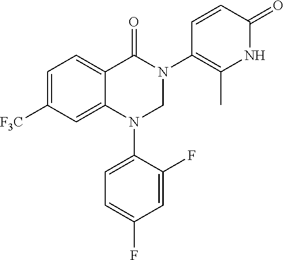 [SMILES: Cc1[nH]c(=O)ccc1N1CN(c2ccc(F)cc2F)c2cc(C(F)(F)F)ccc2C1=O] | 6.6 | 1-(2,4-difluorophenyl)-3-(2- methyl-6-oxo-1,6- dihydropyridin-3-yl)-7- (trifluoromethyl)-2,3- dihydroquinazolin-4(1H)-one |
| 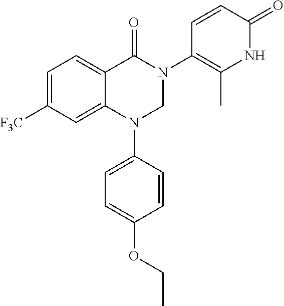 [SMILES: CCOc1ccc(N2CN(c3ccc(=O)[nH]c3C)C(=O)c3ccc(C(F)(F)F)cc32)cc1] | 6.4 | 1-(4-ethoxyphenyl)-3-(2-methyl- 6-oxo-1,6-dihydropyridin-3-yl)- 7-(trifluoromethyl)-2,3- dihydroquinazolin-4(1H)-one |
| 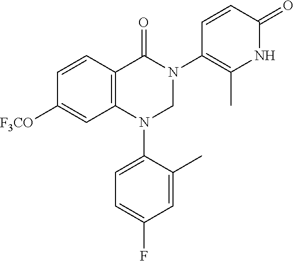 [SMILES: Cc1cc(F)ccc1N1CN(c2ccc(=O)[nH]c2C)C(=O)c2ccc(OC(F)(F)F)cc21] | 8.5 | 1-(4-fluoro-2-methylphenyl)-3- (2-methyl-6-oxo-1,6- dihydropyridin-3-yl)-7- (trifluoromethoxy)-2,3- dihydroquinazolin-4(1H)-one |
| 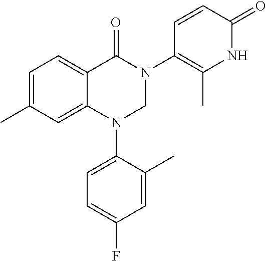 [SMILES: Cc1ccc2c(c1)N(c1ccc(F)cc1C)CN(c1ccc(=O)[nH]c1C)C2=O] | 8.8 | 1-(4-fluoro-2-methylphenyl)-7- methyl-3-(2-methyl-6-oxo-1,6- dihydropyridin-3-yl)-2,3- dihydroquinazolin-4(1H)-one |
| 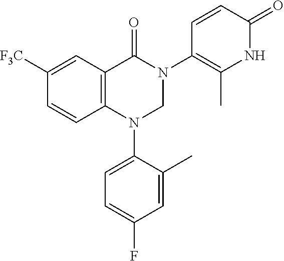 [SMILES: Cc1cc(F)ccc1N1CN(c2ccc(=O)[nH]c2C)C(=O)c2cc(C(F)(F)F)ccc21] | 7.9 | 1-(4-fluoro-2-methylphenyl)-3- (2-methyl-6-oxo-1,6-dihydro pyridine-3-yl)-6- (trifluoromethyl)-2,3- dihydroquinazolin- 4(1H)-one |
| 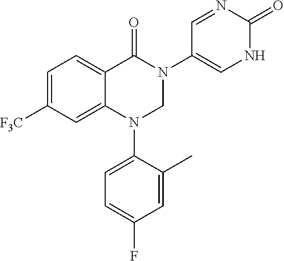 [SMILES: Cc1cc(F)ccc1N1CN(c2cnc(=O)[nH]c2)C(=O)c2ccc(C(F)(F)F)cc21] | 6.3 | 1-(4-fluoro-2-methylphenyl)-3- (2-oxo-1,2-dihydropyrimidin-5- yl)-7-(trifluoromethyl)-2,3- dihydroquinazolin-4(1H)-one |
| 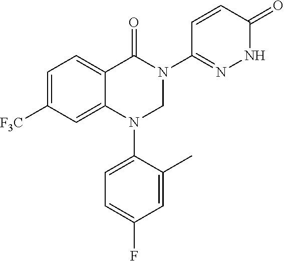 [SMILES: Cc1cc(F)ccc1N1CN(c2ccc(=O)[nH]n2)C(=O)c2ccc(C(F)(F)F)cc21] | 5.7 | 1-(4-fluoro-2-methylphenyl)-3- (6-oxo-1,6-dihydropyridazin-3- yl)-7-(trifluoromethyl)-2,3- dihydroquinazolin-4(1H)-one |
| 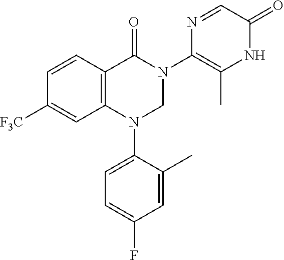 [SMILES: Cc1cc(F)ccc1N1CN(c2ncc(=O)[nH]c2C)C(=O)c2ccc(C(F)(F)F)cc21] | 6.4 | 1-(4-fluoro-2-methylphenyl)-3- (3-methyl-5-oxo-4,5- dihydropyrazin-2-yl)-7- (trifluoromethyl)-2,3- dihydroquinazolin-4(1H)-one |

TABLE 2

| | Naᵥ1.8 | |
|---|---|---|
| Structure | pIC₅₀ | Name |
| 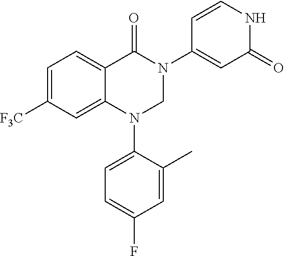 [SMILES: Cc1cc(F)ccc1N1CN(c2cc[nH]c(=O)c2)C(=O)c2ccc(C(F)(F)F)cc21] | 6.4 | 1-(4-fluoro-2-methylphenyl)-3- (2-oxo-1,2-dihydropyridin-4-yl)- 7-(trifluoromethyl)-2,3- dihydroquinazolin-4(1H)-one |
| 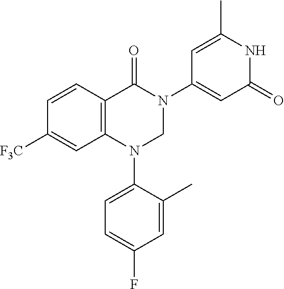 [SMILES: Cc1cc(N2CN(c3ccc(F)cc3C)c3cc(C(F)(F)F)ccc3C2=O)cc(=O)[nH]1] | 5.6 | 1-(4-fluoro-2-methylphenyl)-3- (6-methyl-2-oxo-1,2- dihydropyridin-4-yl)-7- (trifluoromethyl)-2,3- dihydroquinazolin-4(1H)-one |
| 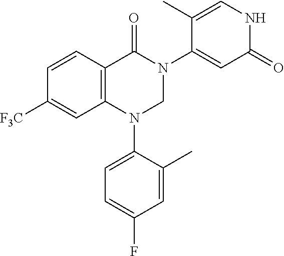 [SMILES: Cc1c[nH]c(=O)cc1N1CN(c2ccc(F)cc2C)c2cc(C(F)(F)F)ccc2C1=O] | 6.0 | 1-(4-fluoro-2-methylphenyl)-3- (5-methyl-2-oxo-1,2- dihydropyridin-4-yl)-7- (trifluoromethyl)-2,3- dihydroquinazolin-4(1H)-one |
| 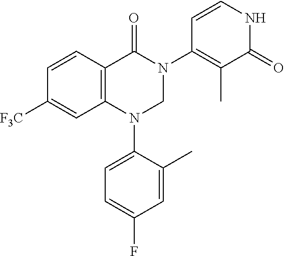 [SMILES: Cc1cc(F)ccc1N1CN(c2cc[nH]c(=O)c2C)C(=O)c2ccc(C(F)(F)F)cc21] | 5.9 | 1-(4-fluoro-2-methylphenyl)-3- (3-methyl-2-oxo-1,2- dihydropyridin-4-yl)-7- (trifluoromethyl)-2,3- dihydroquinazolin-4(1H)-one |

### Compound Examples

The following examples illustrate the invention. These examples are not intended to limit the scope of the present invention, but rather to provide guidance to the skilled artisan to prepare and use the compounds, compositions, and methods of the present invention.

While embodiments of the present invention are described, the skilled artisan will appreciate that various changes and modifications can be made without departing from the spirit and scope of the invention.

It will be understood by the skilled artisan that purification methods (using acidic or basic modifiers) or compound workup procedures (using acidic or basic conditions) may result in formation of a salt of a title compound (for example, hydrobromic acid, formic acid, hydrochloric acid, trifluoroacetic acid, or ammonia salts of a title compound). The present invention is intended to encompass such salts.

Final compounds were characterized with LCMS (conditions listed below) and NMR. ¹H NMR or ¹⁹FNMR spectra were recorded using a Bruker Avance III 500 MHz spectrometer, Bruker Avance 400 MHz spectrometer and Varian Mercury Plus-300 MHz spectrometer. CDCl₃ is deuteriochloroform, DMSO-d₆ is hexadeuteriodimethylsulfoxide, and CD₃OD is tetradeuteriomethanol. Chemical shifts are reported in parts per million (ppm) downfield from the internal standard tetramethylsilane (TMS) or the NMR solvent. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz.

### General

Unless otherwise noted, all starting materials were obtained from commercial suppliers and used without further purification. Unless otherwise indicated, all temperatures are expressed in ° C. (degrees Centigrade). Unless otherwise indicated, all reactions are conducted under an inert atmosphere at ambient temperature.

All temperatures are given in degrees Celsius, all solvents are highest available purity and all reactions run under anhydrous conditions in an argon (Ar) or nitrogen (N₂) atmosphere where necessary.

### Instrumentation

¹H NMR spectra were recorded using a Bruker Avance III 400 MHz spectrometer, BrukerAvance NEO NanoBay V4-3 400 MHz spectrometer. CDCl₃ is deuteriochloroform, DMSO-d₆ is hexadeuteriodimethylsulfoxide, and CD₃OD is tetradeuteriomethanol. Chemical shifts are reported in parts per million (ppm) downfield from the internal standard tetramethylsilane (TMS) or the NMR solvent. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz.

Mass spectra were run on open access LC-MS systems, Waters Acquity QDa mass detector. The compound is analyzed using a reverse phase column, e.g., Xbridge-C18, Sunfire-C18, Thermo Aquasil/Aquasil C18, Acquity HPLC C18, Thermo Hypersil Gold eluted using an acetonitrile and water gradient with a low percentage of an acid modifier such as 0.02% TFA.

### Analytical Methods:

LCMS Method: Acquity UPLC with Waters Acquity QDa mass detector using electrospray positive [ES+ve to give M+H⁺] equipped with a CSH C18 column (30 mm×2.1 mm, i.d. 1.7 μm packing diameter) at 45° C. eluting with 0.1% TFA in water (solvent A) and 0.1% TFA in acetonitrile (solvent B), using the following elution gradient: 1-100% (solvent B) over 1.85 min at a flow rate of 1.3 ml/min.
LCMS Method: Acquity UPLC with Waters Acquity QDa mass detector using electrospray positive [ES+ve to give M+H⁺] equipped with a CSH C18 column (30 mm×2.1 mm, i.d. 1.7 μm packing diameter) at 45° C. eluting with formic acid in Water (solvent A) and formic acid in acetonitrile (solvent B), using the following elution gradient: 1-100% (solvent B) over 1.85 min at a flow rate of 1.3 ml/min.
LCMS Method: Acquity UPLC with Waters Acquity QDa mass detector using electrospray positive [ES+ve to give M+H⁺] equipped with a CSH C18 column (30 mm×2.1 mm, i.d. 1.7 μm packing diameter) at 45° C. eluting with 10 mM ammonium bicarbonate in water adjusted to pH=10 with 25% ammonium hydroxide solution (solvent A) and acetonitrile (solvent B), using the following elution gradient: 1-100% (solvent B) over 1.85 min at a flow rate of 1.3 ml/min.
LCMS method: Agilent 1290 Infinity II LC system with Agilent MSD 6125B/6130 using multi mode (ESI and APCI +ve and −ve) equipped with a Sunfire C18 column (30 mm×2.1 mm, i.d. 3.5 μm packing diameter) at 25° C. eluting with 0.1% Formic acid in water (solvent A) and 0.1% Formic acid in acetonitrile (solvent B), using the following elution gradient: 0-100% (solvent B) over 3.1 min and holding at 100% for 0.8 min at a flow rate of 1.0 ml/min.
LCMS method: Agilent 1290 Infinity II LC system with Agilent MSD 6125B/6130 using multi mode (ESI and APCI +ve and −ve) equipped with a Atlantis dC18 column (50 mm×4.6 mm, i.d. 5.0 μm packing diameter) at 25° C. eluting with 0.1% TFA in water (solvent A) and methanol (solvent B), using the following elution gradient: 5-95% (solvent B) over 5.0 min and holding at 95% for 1.5 min at a flow rate of 1.0 ml/min.
LCMS method: Agilent 1290 Infinity II LC system with Agilent MSD 6125B/6130 using multi mode (ESI and APCI +ve and −ve) equipped with a Zorbax XDB C18 column (50 mm×4.6 mm, i.d. 3.5 μm packing diameter) at 25° C. eluting with 10 mM ammonium acetate in water (solvent A) and acetonitrile (solvent B), using the following elution gradient: Solvent B: 10-95% (solvent B) over 3.5 min and holding at 95% for 1.0 min at a flow rate of 1.0 ml/min.
LCMS method: Agilent 1290 Infinity II LC system with Agilent MSD 6125B/6130 using multi mode (ESI and APCI +ve and −ve) equipped with a Xbridge C8 column (50 mm×4.6 mm, i.d. 3.5 μm packing diameter) at 25° C. eluting with 10 mM ammonium bicarbonate in water (solvent A) and acetonitrile (solvent B), using the following elution gradient: 10-95% (solvent B) over 4.0 min and holding at 95% for 1.0 min at a flow rate of 1.0 ml/min.
LCMS Method: Acquity UPLC with Waters Acquity QDa mass detector using electrospray positive [ES+ve to give M+H⁺] equipped with a CSH C18 column (30 mm×2.1 mm, i.d. 1.7 μm packing diameter) at 55° C. eluting with 0.1% formic acid in Water (solvent A) and 0.1% formic acid in acetonitrile (solvent B), using the following elution gradient: 1-99% (solvent B) over 2.0 min at a flow rate of 1.0 mi/min.

### Example Definitions and Abbreviations

In the following experimental descriptions, the following abbreviations may be used:

| Abbreviation | Meaning |
|---|---|
| ACN or MeCN | acetonitrile |
| AcOH | acetic acid |
| aq. | Aqueous |
| ATM or atm | standard atmosphere |
| BBr₃ | boron tribromide |
| BCl₃ | boron trichloride |
| BH₃ | Borane |
| BINAP | 2,2′-bis(diphenylphosphino)-1,1′-binaphthalene |
| Bn | Benzyl |
| Br₂ | Bromine |
| Brine | saturated aqueous sodium chloride |
| BuLi or nBuLi | butyllithium |
| CDI | carbonyldiimidazole |
| CH₂Cl₂ | methylene chloride |
| CH₃CN | acetonitrile |
| COCl₂ | oxalyl chloride |
| Cs₂CO₃ | cesium carbonate |
| DABAL-Me3 | Bis(trimethylaluminum)-1,4-diazabicyclo[2.2.2]- |
| | octane |
| DAST | Diethylaminosulfur trifluoride |
| DBU | 1,8-Diazabicyclo[5.4.0]undec-7-ene |
| DCC | dicyclohexylcarbodiimide |
| DCM or CH₂Cl₂ | methylene chloride |
| DEAD | diethyl azodicarboxylate |
| DEAP | diethyl aminopyridine |
| DIAD | diisopropyl azodicarboxylate |
| DIPEA, DIEA, | N,N-diisopropylethylamine |
| Hunig's base | |
| DMA | Dimethylacetamide |
| DMAP | 4-dimethylaminopyridine |
| DMF | N,N-dimethylformamide |
| DME | dimethoxyethane |
| DMSO | dimethylsulfoxide |
| EDC | 1-[3-(dimethylamino)propyl]-3-ethylcarbodiimide |
| | hydrochloride |
| Et | Ethyl |
| Et₃N | triethylamine |
| Et₂O | diethyl ether |
| EtOAc | ethyl acetate |
| EtOH | Ethanol |
| Fmoc or fmoc | fluorenylmethyloxycarbonyl |
| g, G, gm, GM | Gram |
| GCMS | gas chromatography-mass spectrometry |
| h or hr | hour(s) |
| H₂ | hydrogen |
| H₂O₂ | hydrogen peroxide |
| H₂O | Water |
| H₂SO₄ | sulfuric acid |
| HATU | (O-(7-azabenzotriazol-1-yl)-N,N,N′,N′- |
| | tetramethyluronium hexafluorophosphate) |
| HBTU | 2-(1H-benzo[d][1,2,3]triazol-1-yl)-1,1,3,3- |
| | tetramethylisouronium hexafluorophosphate(V) |
| HCl | hydrochloric acid |
| HCO₂H | formic acid |
| HOBt or HOBT | 1-hydroxybenzotriazole |
| HPLC | high performance liquid chromatography |
| I₂ | Iodine |
| JLR | jacketed lab reactor |
| K₂CO₃ | potassium carbonate |
| KHSO₄ | potassium hydrogen sulfate |
| KOAc | potassium acetate |
| L or l | Liter |
| LAH | lithium aluminum hydride |
| LCMS | liquid chromatography-mass spectroscopy |
| LDA | lithium diisopropyl amide |
| LED | light-emitting diode |
| LiOH | lithium hydroxide |
| LHMDS | lithium bis(trimethylsilyl)amide |
| mCPBA or | meta-chloroperoxybenzoic acid |
| m-CPBA | |
| MDAP | mass directed auto purification |
| Me | Methyl |
| MeOH | Methanol |
| mg, MG | Milligram |
| MgBr₂ | magnesium bromide |
| MgSO₄ | magnesium sulfate |
| Min or mins | minute(s) |
| ml or mL or ML | Milliliter |
| Mmol | Millimole |
| MnO₂ | manganese dioxide |
| Mol, mol | Mole |
| MS | mass spectrum |
| MTBE | methyl tert-butyl ether |
| μw | Microwave |
| N₂ | Nitrogen |
| Na(CN)BH₃ | sodium cyanoborohydride |
| NaCl | sodium chloride |
| Na₂CO₃ | sodium carbonate |
| NaHCO₃ | sodium bicarbonate |
| NaHMDS | sodium bis(trimethylsilyl)amide |
| NaHSO₃ | sodium bisulfite |
| NaH | sodium hydride |
| NaI | sodium iodide |
| NaOH | sodium hydroxide |
| Na₂SO₃ | sodium sulfite |
| Na₂SO₄ | sodium sulfate |
| NBS | N-Bromosuccinimide |
| NH₄CI | ammonium chloride |
| HCO₂•NH₄ | ammonium formate |
| NH₄OH | ammonium hydroxide |
| Nm | nanometer |
| NMO | 4-methylmorpholine N-oxide |
| NMP | N-methyl-2-pyrrolidone |
| Pet. | Petroleum ether |
| Pd/C or Pd-C | palladium on carbon |
| PdCl₂(dbpf) | 1,1′-bis(di-tert-butylphosphino)ferrocene |
| | dichloropalladium |
| Pd(dppf)Cl₂/ | [1,1′-bis(diphenylphosphino)ferrocene] |
| PdCl₂(dppf) | dichloropalladium(II) |
| PdCl₂(dppf)- | [1,1′-bis(diphenylphosphino)ferrocene] |
| CH₂Cl₂ | dichloropalladium(II), complex with dichloromethane |
| adduct | |
| PdCl₂(Xantphos) | Dichloro[9,9-dimethyl-4,5- |
| | bis(diphenylphosphino)xanthene]palladium(II) |
| Pd₂(dba)₃ | tris(dibenzylideneacetone)dipalladium(0) |
| Pd(Ph₃)_{4,} | tetrakis(triphenylphosphine)palladium(0) |
| tetrakis | |
| [Pd(Pi- | Palladium(1-phenylallyl)chloride dimer |
| Cinnamyl)Cl]₂ | |
| Pd(OAc)₂ | palladium acetate or Palladium(II) acetate |
| Pd(OH)₂ | palladium hydroxide |
| PIFA | [Bis(trifluoroacetoxy)iodo]benzene |
| Ph | Phenyl |
| PL HCO₃ MP | macroporus polystyrene supported carbonate |
| POCl₃ | phosphoryl chloride |
| Psi | Pounds per square inch |
| Pt/C | Platinum on carbon |
| PTFE | Polytetrafluoroethylene |
| PTSOH or PTSA | p-Toluenesulfonic acid |
| or pTsOH | |
| rt or RT | room temperature or retention time (when use with |
| | chromatography) |
| sat. | Saturated |
| SFC | supercritical fluid chromatography |
| Si | Silica |
| Si SPE | silica gel cartridges |
| SiO₂ | silica gel |
| SPE | solid phase extraction |
| T3P ® | propylphosphonic anhydride |
| tBu or t-Bu | tert-butyl group |
| TBAB | tetrabutylammonium bromide |
| TBAF | tetrabutylammonium fluoride |
| TBAI | tetrabutylammonium iodide |
| TBDMS-Cl | tert-butyldimethylsilyl chloride |
| TBME | tert-butylmethyl ether |
| TBS | tert-butyldimethylsilyl |
| TBTU | 2-(1H-benzotriazole-1-yl)-1,1,3,3-tetramethyluronium |
| | tetrafluoroborate |
| (t-Bu)PhCPhos | 2-[(tert-Butyl)phenylphosphino]-2′,6′-bis(N,N- |
| | dimethylamino)biphenyl |
| tBuXphos | 2-Di-tert-butylphosphino-2′,4′,6′-triisopropylbiphenyl |
| TEA | Triethylamine |
| TFA | trifluoroacetic acid |
| THF | Tetrahydrofuran |
| TiCl₄ | titanium tetrachloride |
| TMS-Br or | trimethylsilyl bromide |
| TMSBr | |
| TMS-Cl or | trimethylsilyl chloride |
| TMSCl | |
| TMSI | lodotrimethylsilane or trimethylsilyl iodide |
| TMS-OTf | trimethylsilyl tritiate |
| or TMSOtf | |
| tR | retention time |
| UPLC | ultra performance liquid chromatography |
| Xantphos | 4,5-Bis(diphenylphosphino)-9,9-dimethylxanthene |
| Xphos | 2-Dicyclohexylphosphino-2′,4′,6′-triisopropylbiphenyl |

### Intermediate Compound Examples

### Intermediate 1

### 6-Amino-3-chloro-2,4-difluorobenzoic Acid

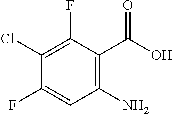 [SMILES: Nc1cc(F)c(Cl)c(F)c1C(=O)O]

N-chlorosuccinimide (3.23 g, 24.21 mmol) was added dropwise to a stirring mixture of 2-amino-4,6-difluorobenzoic acid (3.81 g, 22.01 mmol) under N₂ at 60° C. The reaction mixture was stirred at 60° C. for 2 hours. The reaction was diluted with water (200 mL) and extracted with EtOAc (3×100 mL). The combined organic layers were washed with brine (100 mL), dried over Na₂SO₄, filtered and concentrated to give the title compound as a yellow solid (2.3 g, 11.17 mmol, 51% yield). MS (m/z) 208.2 (M+H)⁺.

Intermediate 2 was prepared from the indicated aryl aniline by methods analogous to those described for Intermediate 1

| Int. | Name | Structure | Characterization | Aryl Aniline |
|---|---|---|---|---|
| 2 | methyl 6-amino-4- bromo-3-chloro-2- fluorobenzoate | 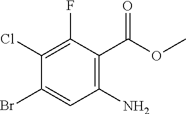 | MS (m/z) 282.0 (M + H)⁺ | methyl 2- amino-4- bromo-6- fluorobenzoate |

### Intermediate 3

### 2-Bromo-4-(methylsulfonyl)benzoic Acid

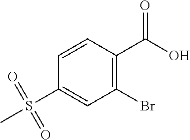

To a stirring suspension of 2-amino-4-(methylsulfonyl)benzoic acid (500 mg, 2.323 mmol) in HBr (48% in water) (8 mL, 147 mmol) at 0° C. was added a solution of sodium nitrite (192 mg, 2.79 mmol) in water (0.6 mL) dropwise under the surface. After stirring at 0° C. for 10 minutes, copper(I) bromide (400 mg, 2.79 mmol) was added in small portions at the same temperature. The reaction mixture was warmed to 30° C. and water (5.0 mL) was added to facilitate stirring. The resulting reaction mixture was stirred at 30° C. for 17 hours and poured into a saturated aqueous Na₂CO₃ solution (50 mL). The resulting blue solution was acidified to pH=1 with 12 N HCl at 0° C. and extracted with EtOAc (2×50 mL). The organic extracts were dried over Na₂SO₄ and concentrated in vacuo to give the title compound as an off-white solid (650 mg, 2.214 mmol, 95% yield). MS (m/z) 276.8 (M−H)⁻

### Intermediate 4

### 2-Bromo-5-methoxy-4-(trifluoromethyl)benzoic Acid

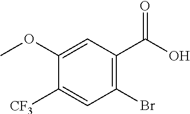 [SMILES: COc1cc(C(=O)O)c(Br)cc1C(F)(F)F]

To a solution of 3-methoxy-4-(trifluoromethyl)benzoic acid (2 g, 9.08 mmol) in a mixture of acetic acid (20 mL) and water (20 mL) stirred at room temperature was added bromine (0.468 mL, 9.08 mmol). The reaction mixture was stirred at 80° C. for 16 h. The reaction mixture was quenched with water. The solid was filtered and dried under vacuum for 2 hours to give the title compound as a white solid (2.1 g, 46% purity, 3.23 mmol, 35.6% yield). MS (m/z) 296.8 (M−H)⁻.

### Intermediate 5

### Ethyl 2-bromo-4-(trifluoromethyl)benzoate

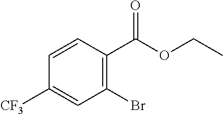 [SMILES: CCOC(=O)c1ccc(C(F)(F)F)cc1Br]

To a stirring solution of 2-bromo-4-(trifluoromethyl)benzoic acid (10.0 g, 37.2 mmol) in DMF (100 mL) was added K₂CO₃ (5.65 g, 40.9 mmol) followed by ethyl iodide (3.60 mL, 44.6 mmol) dropwise under N₂ at 25° C. The reaction mixture was stirred at the same temperature for 3 hours. Water (150 mL) was added and the reaction was extracted with EtOAc (2×250 mL). The combined organic extracts were washed with brine (150 mL), dried over Na₂SO₄, filtered, and concentrated. The residue was purified by column chromatography (Biotage, 100 g SNAP column, 10% EtOAc/petroleum ether over 40 minutes) to give the title compound as a colorless oil (9.3 g, 31.3 mmol, 84% yield). GCMS (m/z) 296.0 (M+H)⁺.

Intermediates 6-25 were prepared from the indicated carboxylic acid by methods analogous to those described for Intermediate 5.

| Int. | Name | Structure | Characterization | Acid |
|---|---|---|---|---|
| 6 | ethyl 2-bromo-5- chlorobenzoate | 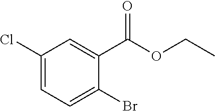 | ¹H NMR (400 MHz, CDCl₃) δ: 7.78 (d, J = 2.6 Hz, 1H), 7.60 (d, J = 8.6 Hz, 1H), 7.32 (dd, J = 8.6, 2.6 Hz, 1H), 4.42 (q, J = 7.1 Hz, 2H), 1.43 (t, J = 7.1 Hz, 3H) | 2-bromo-5- chlorobenzoic acid |
| 7 | ethyl 2-bromo-5- methylbenzoate | 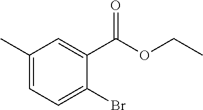 | ¹HNMR (400 MHz, DMSO-d₆) δ: 7.61 (d, J = 8.1 Hz, 1H), 7.56 (d, J = 1.6 Hz, 1H), 7.32- 7.26 (m, 1H), 4.32 (q, J = 7.0 Hz, 2H), 2.31 (s, 3H), 1.32 (t, J = 7.1 Hz, 3H) | 2-bromo-5- methylbenzoic acid |
| 8 | ethyl 2-bromo-6- (trifluoromethyl) nicotinate | 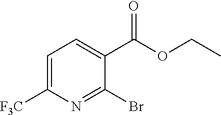 [SMILES: CCOC(=O)c1ccc(C(F)(F)F)nc1Br] | MS (m/z) 300.0 (M + H)⁺ | 2-bromo-6- (trifluoromethyl) nicotinic acid |
| 9 | ethyl 2-bromo-5- (trifluoromethyl) benzoate | 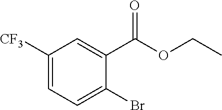 [SMILES: CCOC(=O)c1cc(C(F)(F)F)ccc1Br] | ¹HNMR (400 MHz, DMSO-d₆) δ: 8.08 (d, J = 2.3 Hz, 1H), 8.02 (d, J = 8.4 Hz, 1H), 7.85 (dd, J = 8.4, 1.9 Hz, 1H), 4.37 (q, J = 7.1 Hz, 2H), 1.34 (t, J = 7.1 Hz, 3H) | 2-bromo-5- (trifluoromethyl) benzoic acid |
| 10 | ethyl 2-bromo-6- (trifluoromethyl) benzoate | 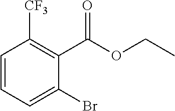 [SMILES: CCOC(=O)c1c(Br)cccc1C(F)(F)F] | GCMS (m/z) 296.0 (M + H)⁺ | 2-bromo-6- (trifluoromethyl) benzoic acid |
| 11 | ethyl 2-bromo-4- chlorobenzoate | 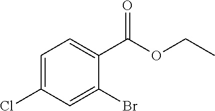 | GCMS (m/z) 263.9 (M + H)⁺ | 2-bromo-4- chlorobenzoic acid |
| 12 | methyl 6-amino-3- chloro-2,4- difluorobenzoate | 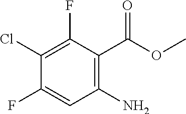 | MS (m/z) 222.1 (M + H)⁺ | 6-amino-3- chloro-2,4- difluorobenzoic acid |
| 13 | methyl 2-chloro-5- (trifluoromethyl) nicotinate | 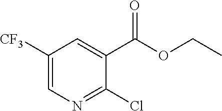 | GCMS (m/z) 239.0 (M + H)⁺ | 2-chloro-5- (trifluoromethyl) nicotinic acid |
| 14 | ethyl 2-bromo-4- methylbenzoate | 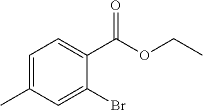 | MS (m/z) 244.8 (M + 3H)⁺ | 2-bromo-4- methylbenzoic acid |
| 15 | ethyl 2-bromo-5- cyanobenzoate | 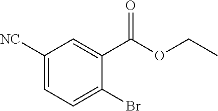 | GCMS (m/z) 252.9 (M)⁺ | 2-bromo-5- cyanobenzoic acid |
| 16 | ethyl 2-bromo-4- cyanobenzoate | 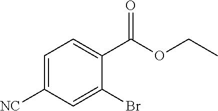 | GCMS (m/z) 252.9 (M)⁺ | 2-bromo-4- cyanobenzoic acid |
| 17 | ethyl 2-bromo-4- (methylsulfonyl) benzoate | 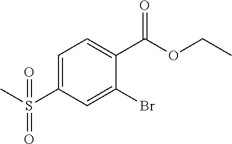 [SMILES: CCOC(=O)c1ccc(S(C)(=O)=O)cc1Br] | GCMS (m/z) 305.9 (M)⁺ | 2-bromo-4- (methylsulfonyl) benzoic acid |
| 18 | ethyl 2-bromo-4- nitrobenzoate | 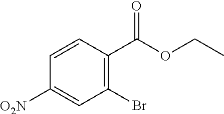 | MS (m/z) 272.8 (M)⁻ | 2-bromo-4- nitrobenzoic acid |
| 19 | ethyl 2-bromo-6- chlorobenzoate | 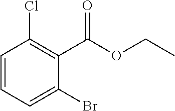 | GCMS (m/z) 263.9 (M + H)⁺ | 2-bromo-6- chlorobenzoic acid |
| 20 | ethyl 2-bromo-5- methoxy-4- (trifluoromethyl) benzoate | 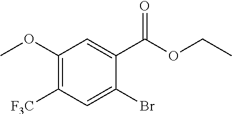 [SMILES: CCOC(=O)c1cc(OC)c(C(F)(F)F)cc1Br] | MS (m/z) 326.0 (M + H)⁺ | 2-bromo-5- methoxy-4- (trifluoromethyl) benzoic acid |
| 21 | ethyl 2-bromo-4- methoxybenzoate | 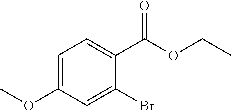 | MS (m/z) 258.9 (M + H)⁺ | 2-bromo-4- methoxybenzoic acid |
| 22 | ethyl 2-bromo-6- methoxybenzoate | 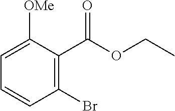 | MS (m/z) 260.9 (M + 3H)⁺ | 2-bromo-6- methoxybenzoic acid |
| 23 | methyl 2-chloro-6- (difluoromethyl) nicotinate | 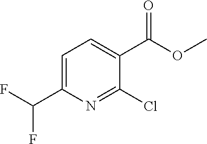 | MS (m/z) 222.0 (M + H)⁺ | 2-chloro-6- (difluoromethyl) nicotinic acid |
| 24 | ethyl 2-bromo-3- chlorobenzoate | 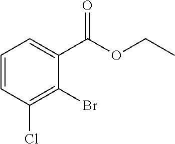 | GCMS (m/z) 264.0 (M + H)⁺ | 2-bromo-3- chlorobenzoic acid |
| 25 | ethyl 2-bromo-6- methylbenzoate | 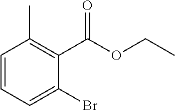 | MS (m/z) 245.0 (M + 3H)⁺ | 2-bromo-6- methylbenzoic acid |

### Intermediate 26

### Methyl 2-bromo-5-(trifluoromethyl)benzoate

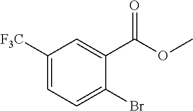

To a stirring solution of 2-bromo-5-(trifluoromethyl)benzoic acid (750 mg, 2.79 mmol) in methanol (3 mL) was added sulfuric acid (0.4 mL, 7.50 mmol) resulting in an exotherm.

The resulting solution was heated in sealed vial at 70° C. for 2.5 hours. The cooled reaction mixture was diluted with water followed by extraction into 2 portions of TBME. The combined organic extracts were dried by filtration through a hydrophobic frit and concentrated under a stream of nitrogen to give the title compound as a pale yellow oil (716 mg, 2.53 mmol, 91% yield). ¹H NMR (400 MHz, CDCl₃) δ: 8.06 (d, J=1.7 Hz, 1H), 7.81 (d, J=8.3 Hz, 1H), 7.59-7.55 (m, 1H), 3.97 (s, 3H).

Intermediates 27-29 were prepared from the indicated carboxylic acid by methods analogous to those described for Intermediate 26

| Int. | Name | Structure | Characterization | Acid |
|---|---|---|---|---|
| 27 | ethyl 2-chloro-6- hydroxynicotinate | 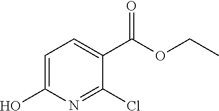 | ¹H NMR (400 MHz, DMSO d-₆) δ (ppm) = 8.04 (d, J = 8.6 Hz, 1H), 6.58 (d, J = 8.6 Hz, 1H), 4.26 (q, J = 7.1 Hz, 2H), 1.29 (t, J = 7.1 Hz, 3H) | 2-chloro-6- hydroxynicotinic acid |
| 28 | methyl 2-amino-5- (trifluoromethyl) nicotinate | 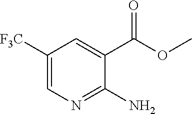 | MS (m/z) 221.2 (M + H)⁺. | 2-amino-5- (trifluoromethyl) nicotinic acid |
| 29 | ethyl 2-amino-5- chlorobenzoate | 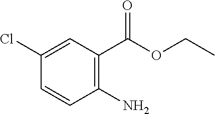 | MS (m/z) 200.1 (M + H)⁺ | 2-amino-5- chlorobenzoic acid |

### Intermediate 30

### Ethyl 2-bromo-4-(2,2,2-trifluoroethoxy)benzoate

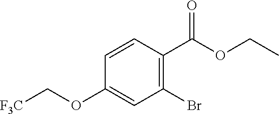 [SMILES: CCOC(=O)c1ccc(OCC(F)(F)F)cc1Br]

### Step 1: Ethyl 4-amino-2-bromobenzoate

To a stirring solution of ethyl 2-bromo-4-nitrobenzoate (14.6 g, 53.3 mmol) in Isopropanol (40 mL) and water (160 mL) were added ammonium chloride (3.42 g, 63.9 mmol) and iron (17.85 g, 320 mmol) at 0° C. After stirring at 100° C. for 2 hr., the reaction was allowed to cool to RT and filtered through a Celite pad washing with EtOAc (500 mL).

The filtrate was washed with water (200 mL) and brine (100 mL), dried over Na₂SO₄ and concentrated in vacuo to give the title compound as an off-white solid (12.4 g, 50.2 mmol, 94% yield). MS (m/z) 244.0 (M+H)⁺.

### Step 2: Ethyl 2-bromo-4-hydroxybenzoate

To a stirring solution of ethyl 4-amino-2-bromobenzoate (12.4 g, 50.8 mmol) in water (120 mL) was added sulfuric acid (12.40 mL, 233 mmol) dropwise over 5 minutes at 0° C. After stirring for 5 minutes, a solution of sodium nitrite (3.51 g, 50.8 mmol) in water (30 mL) was added dropwise over 15 min and the reaction mixture was stirred at 0° C. for 2 hr. The reaction was filtered and the filter cake was washed with water (100 mL). The filtrate was heated to reflux for 1 hr and then stirred at RT for 16 hr. The reaction was extracted with EtOAc (2×150 mL), washed with brine (100 mL), dried over Na₂SO₄ and concentrated in vacuo. The crude product was purified by column chromatography (Biotage, 100 g SNAP column, 0-50% EtOAc/petroleum ether over 40 minutes) to give the title compound as a red solid (7.4 g, 26.7 mmol, 52.6% yield). MS (m/z) 245.0 (M+H)⁺.

### Step 3: Ethyl 2-bromo-4-(2,2,2-trifluoroethoxy)benzoate

To a stirring solution of ethyl 2-bromo-4-hydroxybenzoate (2.0 g, 8.16 mmol) in DMSO (20 mL) was added K₂CO₃ (1.692 g, 12.24 mmol) under N₂ at RT. After stirring for 15 minutes, 1,1,1-trifluoro-2-iodoethane (2.413 mL, 24.48 mmol) was added dropwise and the resulting reaction mixture was stirred at 100° C. under N₂ for 22 hours. The reaction mixture was allowed to cool to RT, quenched with water (100 mL) and extracted with EtOAc (3×100 mL). The combined organic extracts were washed with water (100 mL) and brine (100 mL), dried over Na₂SO₄ and concentrated. The crude product was purified by column chromatography (Isolera, 100 g SNAP column, 0-25% EtOAc/petroleum ether over 40 minutes to give the title compound as a colorless liquid (1.5 g, 4.47 mmol, 54.8% yield). ¹HNMR (400 MHz, DMSO-d₆): δ 7.82 (d, J=8.40 Hz, 1H), 7.48 (d, J=2.80 Hz, 1H), 7.19 (dd, J=8.80, 2.40 Hz, 1H), 4.92 (q, J=8.80 Hz, 2H), 4.30 (q, J=6.80 Hz, 2H), 1.32 (t, J=7.20 Hz, 3H).

### Intermediate 31

### Methyl 6-bromo-3-chloro-2-methylbenzoate

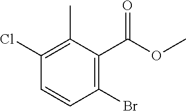

### Step 1: Methyl 3-amino-6-bromo-2-methylbenzoate

To a stirring solution of methyl 3-amino-2-methylbenzoate (5 g, 30.3 mmol) in acetic acid (100 mL) and methanol (200 mL) under N₂ at 0° C., bromine (1.560 mL, 30.3 mmol) was added dropwise. After stirring at 0° C. for 15 minutes, the reaction mixture was quenched with water (200 mL) and concentrated under reduced pressure. The residue was dissolved in DCM (200 mL), washed with saturated NaHCO₃ solution (100 mL) and brine (100 mL), dried over Na₂SO₄ and concentrated in vacuo. The brown liquid residue was purified by column chromatography (Biotage, 100 g SNAP column, 0-23% EtOAc/petroleum ether over 60 min) to give the title compound as an orange gum (2.3 g, 9.23 mmol, 30.5% yield). MS (m/z) 244.0 (M+H)⁺.

### Step 2: Methyl 6-bromo-3-chloro-2-methylbenzoate

To a stirring solution of copper(II) chloride (2.53 g, 18.85 mmol) and tert-butyl nitrite (3.31 mL, 28.3 mmol) in acetonitrile (20 mL) was added a solution of methyl 3-amino-6-bromo-2-methylbenzoate (2.3 g, 9.42 mmol) in acetonitrile (20 mL) dropwise under N₂ at 0° C. The resulting reaction mixture was slowly warmed to 30° C. and stirred for 16 hr at the same temperature. The reaction was quenched with water (50 mL) and extracted with EtOAc (2×25 mL). The combined organic extracts were washed with water (25 mL) and brine (25 mL), dried over Na₂SO₄ and concentrated in vacuo. The brown liquid residue was purified by column chromatography (Biotage, 25 g SNAP column, 0-10% EtOAc/pet ether over 40 min) to give the title compound as an orange liquid (1.75 g, 6.49 mmol, 68.8% yield). GCMS (m/z) 264.0 (M+H)⁺.

### Intermediate 32

### Methyl 6-amino-3-chloro-4-cyano-2-fluorobenzoate

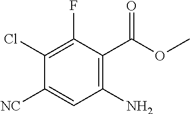

A mixture of methyl 6-amino-4-bromo-3-chloro-2-fluorobenzoate (1.10 g, 3.89 mmol) and copper(I) cyanide (0.698 g, 7.79 mmol) in DMF (20.00 mL) was stirred at 140° C. overnight. The reaction was cooled to RT and diluted with sat. Na₂CO₃ aqueous solution (100 mL). The mixture was extracted with EtOAc (3×100 mL). The combined organic layer was washed with brine (100 mL), dried over Na₂SO₄, filtered and concentrated in vacuo. The residue was purified by column chromatography (Isco, 40 g RediSep Rf Gold high performance flash columns, 0-30% EtOAc/heptane over 30 minutes) to give the title compound as a yellow solid (0.28 mg, 1.2 mmol, 31% yield). MS (m/z) 229.2 (M+H)⁺.

Intermediate 33 was prepared from the indicated aryl halogen by methods analogous to those described for Intermediate 32

| Int. | Name | Structure | Characterization | Aryl halogen |
|---|---|---|---|---|
| 33 | methyl 2-amino-4- chloro-5- cyanobenzoate | 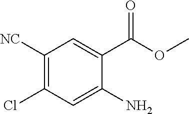 | MS (m/z) 211.0 (M + H)⁺ | methyl 2- amino-4- chloro-5- iodobenzoate |

### Intermediate 34

### Ethyl 2-chloro-6-(difluoromethoxy)nicotinate

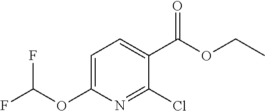 [SMILES: CCOC(=O)c1ccc(OC(F)F)nc1Cl]

To a stirred suspension of ethyl 2-chloro-6-hydroxynicotinate (200 mg, 0.992 mmol) and sodium sulfate (300 mg, 2.112 mmol) in acetonitrile (10 mL) at ambient temperature was added 2,2-difluoro-2-(fluorosulfonyl)acetic acid (0.164 mL, 1.587 mmol). After 2 hours, further 2,2-difluoro-2-(fluorosulfonyl)acetic acid (0.1 mL, 0.968 mmol) was added and stirring continued for a further 45 minutes. The reaction mixture was diluted with NaHCO₃ (aq.) and brine followed by extraction into 2 portions of EtOAc. The combined extracts were washed with brine and dried by filtration through a hydrophobic frit. The filtrate was partially concentrated under reduced pressure (water bath at 30° C. and vacuum pressure >115 mBar) to yield the title compound yellow oil. Yield assumed as 100%. MS (m/z) 252 (M+H)⁺.

### Intermediate 35

### Methyl 2-bromo-5-cyano-4-(trifluoromethyl)benzoate

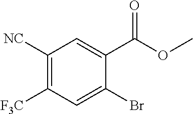 [SMILES: COC(=O)c1cc(C#N)c(C(F)(F)F)cc1Br]

To a solution of 2-bromo-5-cyano-4-(trifluoromethyl)benzoic acid (2000 mg, 6.80 mmol) in methanol (20 mL) was added thionyl chloride (1.489 mL, 20.41 mmol) and the reaction mixture was heated at 70° C. for 1 hr. Solvent was removed and the reaction was extracted with EtOAc (3×50 mL), dried over Na₂SO₄ and concentrated to give the title compound as a light yellow solid (1.96 g, 6.11 mmol, 90% yield). ¹H NMR (400 MHz, CHLOROFORM-d) δ: 8.26 (s, 1H), 8.13 (s, 1H), 4.03 (s, 3H).

Intermediates 36-38 were prepared from the indicated carboxylic acid by methods analogous to those described for Intermediate 35.

| Int. | Name | Structure | Characterization | Acid |
|---|---|---|---|---|
| 36 | Methyl 2-bromo- 5-chloro-4- methylbenzoate | 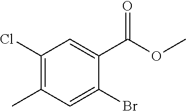 | ¹H NMR (400 MHz, CHLOROFORM-d) δ ppm 7.85 (s, 1H) 7.57 (s, 1H) 3.94 (s, 3H) 2.41 (s, 3H) | 2-bromo-5- chloro-4- methylbenzoic acid |
| 37 | methyl 2,6- dichloro-5- fluoronicotinate | 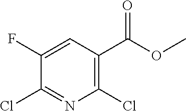 | MS (m/z) 224.0 (M + H)⁺ | 2,6-dichloro- 5- fluoronicotinic acid |
| 38 | methyl 2,5,6- trichloronicotinate | 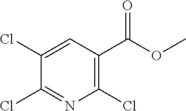 | GCMS (m/z) 238.9 (M)⁺ | 2,5,6- trichloronicotinic acid |

### Intermediate 39

### Methyl 2-chloro-5-fluoro-6-methoxynicotinate

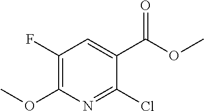

### Step 1: Methyl 2,6-dichloro-5-fluoronicotinate

To a solution of 2,6-dichloro-5-fluoronicotinic acid (5.00 g, 23.81 mmol) in methanol (29.8 ml) was added concentrated HCl (1.955 ml, 23.81 mmol). The solution was heated to 60° C. for 21 hours. H₂SO₄ (1.269 ml, 23.81 mmol) was then added and heated for 20 hours. The solvent was concentrated and the residue was diluted with EtOAc, washed with water (3×), saturated NaHCO₃, brine and dried with MgSO₄ and concentrated under reduced pressure to provide the title compound (5.00 g, 22.10 mmol, 93% yield). MS (m/z) 224 (M+H)⁺.

### Step 2: Methyl 2-chloro-5-fluoro-6-methoxynicotinate

To a solution of methyl 2,6-dichloro-5-fluoronicotinate (1.05 g, 4.69 mmol) in methanol (7.81 ml) was added sodium methoxide (10.31 ml, 5.16 mmol) and the solution was heated to 60° C. for 1 hour. The reaction was cooled and quenched with water. The solvent was concentrated and the residue was suspended between DCM and water. The layers were separated and the aqueous layer was extracted with DCM (3×). The combined organics were washed with water, brine and dried with MgSO₄. The solvent was concentrated under reduced pressure to provide the title compound (0.946 g, 4.31 mmol, 92% yield). MS (m/z) 220 (M+H)⁺.

### Intermediate 40

### Methyl 2-bromo-5-chloro-4-formylbenzoate

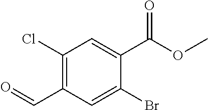

### Step 1: Methyl 2-bromo-4-(bromomethyl)-5-chlorobenzoate

To a solution of methyl 2-bromo-5-chloro-4-methylbenzoate (1.3 g, 4.93 mmol) in DCE (10 mL) was added NBS (1.054 g, 5.92 mmol) and the reaction mixture was heated at 80° C. overnight. The reaction mixture was diluted with DCM (50 ml), washed with aq. NaHCO₃, water and brine and dried over Na₂SO₄. Solvent was removed and the crude product was purified by column chromatography (Isco, 0-30% EtOAc/hexanes) to provide the title compound as a yellow solid (1.2 g, 3.40 mmol, 68.9% yield). ¹H NMR (400 MHz, CHLOROFORM-d) δ: 7.87 (s, 1H), 7.76 (s, 1H), 4.53 (s, 2H) 3.96 (s, 3H).

### Step 2: Methyl 2-bromo-5-chloro-4-formylbenzoate

To a solution of methyl 2-bromo-4-(bromomethyl)-5-chlorobenzoate (200 mg, 0.467 mmol) in DCM (3 mL) were added trimethylamine oxide (140 mg, 1.869 mmol) and DMSO (1.1 ml, 15.50 mmol) at 0° C. The reaction mixture was heated at 30° C. overnight. Water was added and the reaction was extracted with Et₂O, dried over MgSO₄ and concentrated.

The crude product was purified by column chromatography (Isco, 0-20% EtOAc/hexanes) to provide the title compound (72 mg, 0.259 mmol, 55.5% yield). ¹H NMR (400 MHz, DMSO-d₆) δ: 10.19-10.31 (m, 1H) 8.09 (s, 1H) 8.01 (s, 1H) 3.91 (s, 3H).

### Intermediate 41

### Methyl 2-bromo-5-fluoro-4-formylbenzoate

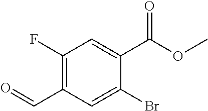

This intermediate was prepared from methyl 2-bromo-5-fluoro-4-methylbenzoate by methods analogous to those described for Intermediate 40. In step 2, potassium bicarbonate was used in place of trimethylamine oxide. GCMS (m/z) 259.9 (M)⁺.

### Intermediate 42

### Ethyl 2-bromo-6-formylbenzoate

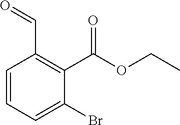

### Step 1: Ethyl 2-bromo-6-(dibromomethyl)benzoate

To a solution of ethyl 2-bromo-6-methylbenzoate (2 g, 8.23 mmol) and benzoyl peroxide (0.598 g, 2.468 mmol) in chlorobenzene (10 mL) under nitrogen at RT was added NBS (4.39 g, 24.68 mmol). The reaction mixture was stirred at 80° C. for 16 h then concentrated under reduced pressure. The residue was purified by column chromatography (Biotage, 50 g SNAP column, 5% EtOAc/95% hexanes over 30 mins) to give the title compound as a brown oil (2.9 g, 5.53 mmol, 67.2% yield). MS (m/z) 400.0 (M+H)⁺.

### Step 2: Ethyl 2-bromo-6-formylbenzoate

To a solution of ethyl 2-bromo-6-(dibromomethyl)benzoate (2 g, 4.99 mmol) in isopropanol (20 mL) and water (4 mL) under nitrogen at room temperature was added silver nitrate (1.695 g, 9.98 mmol). The reaction mixture was stirred at RT for 5 h. The reaction mixture was filtered through a bed of Celite and the Celite was washed with DCM (2×50 ml). The filtered organic layer was washed with water (100 mL). The combined organics were dried over sodium sulphate and concentrated under reduced pressure. The residue was purified by column chromatography (Biotage, 25 g SNAP column, 30% EtOAc/70% hexanes over 30 mins) to give the title compound as a colorless oil (750 mg, 2.477 mmol, 49.6% yield). MS (m/z) 257.0 (M+H)⁺.

### Intermediate 43

### Methyl 2-bromo-5-chloro-4-(difluoromethyl)benzoate

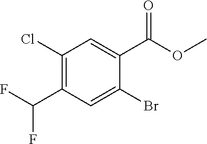

### Step 3: Methyl 2-bromo-5-chloro-4-(difluoromethyl)benzoate

To a solution of methyl 2-bromo-5-chloro-4-formylbenzoate (1200 mg, 4.32 mmol) in DCM (20 ml) was added DAST (1.714 ml, 12.97 mmol) dropwise at 0° C. The reaction mixture was stirred at room temperature for 3 hours. Solvent was removed and the crude product was purified by column chromatography (Isco, 40 g column, 0-20% EtOAc/hexanes) to provide the title compound (1.15 g, 3.65 mmol, 84% yield). ¹H NMR (400 MHz, CHLOROFORM-d) δ: 7.94-8.01 (m, 1H) 7.87 (t, J=1.22 Hz, 1H), 6.92 (t, J=52.82 Hz, 1H), 3.99 (s, 3H).

Intermediates 44-46 were prepared from the indicated aldehyde by methods analogous to those described for Intermediate 43.

| Int. | Name | Structure | Characterization | Aldehyde |
|---|---|---|---|---|
| 44 | methyl 2-bromo- 4- (difluoromethyl)- 5-fluorobenzoate | 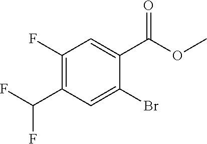 | GCMS (m/z) 282.0 (M + H)⁺ | methyl 2-bromo- 5-fluoro-4- formylbenzoate |
| 45 | ethyl 2-bromo-6- (difluoromethyl)benzoate | 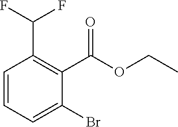 | GCMS (m/z) 278.0 (M + H)⁺ | ethyl 2-bromo-6- formylbenzoate |
| 46 | methyl 2-bromo-4- (difluoromethyl)benzoate | 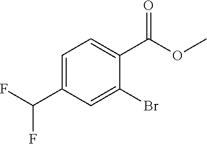 | ¹H NMR(400 MHz, DMSO-d₆) δ: 7.88 (d, J = 8.00 Hz, 1H), 7.83 (d, J = 0.40 Hz, 1H), 7.53 (td, J = 8.00, 0.40 Hz, 1H), 6.66 (t, J = 55.60 Hz, 1H), 3.98 (s, 3H) | methyl 2-bromo- 4-formylbenzoate |

### Intermediate 47

### Methyl 2,5-dichloro-6-cyanonicotinate

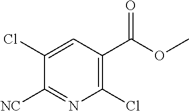

### Step 1: 2,5-Dichloro-3-(methoxycarbonyl)pyridine 1-oxide

To a solution containing methyl 2,5-dichloronicotinate (10 g, 48.5 mmol) in TFA (60 mL) was added hydrogen peroxide 30% (10 ml, 98 mmol). The reaction was warmed to 70° C. for 1 hr at which time the reaction was concentrated onto Celite. Purification by column chromatography on Isco, SiO₂ (120 g with 0-100% EtOAc/heptane as eluant) afforded the title compound as a colorless solid (5.66 g, 25.5 mmol, 52.5% yield). MS (m/z) 222.1 (M+H)⁺.

### Step 2: Methyl 2,5-dichloro-6-cyanonicotinate

To a solution containing 2,5-dichloro-3-(methoxycarbonyl)pyridine 1-oxide (5.66 g, 25.5 mmol) in acetonitrile (50 ml) was added triethylamine (5.33 ml, 38.2 mmol) followed by TMS-CN (8.54 ml, 63.7 mmol). The reaction was warmed to 70° C. for 20 minutes at which time the reaction was cooled to RT, diluted with EtOAc, quenched with cold K₂CO₃ solution, extracted with DCM. Combined organic layers were washed with brine, dried over MgSO₄, filtered and concentrated. Purification by column chromatography on Isco on SiO₂ (120 g with 0-50% EtOAc/heptane as eluant) afforded the title compound as a colorless solid (5.33 g, 23.07 mmol, 90% yield). MS (m/z) 231.2 (M+H)⁺.

### Intermediate 48

### 5-Chloro-4-(difluoromethoxy)-2-fluorobenzonitrile

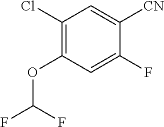 [SMILES: N#Cc1cc(Cl)c(OC(F)F)cc1F]

To a solution of 5-chloro-2-fluoro-4-hydroxybenzonitrile (1 g, 5.83 mmol) in DMF (12 mL) and water (1.2 mL) was added K₂CO₃ (1.208 g, 8.74 mmol) and sodium 2-chloro-2,2-difluoroacetate (2.222 g, 14.57 mmol). After heating at 100° C. for 5 hours, the reaction mixture was allowed to cool to room temperature and extracted with EtOAc (3×20 ml). The combined organic extracts were washed with water (2×10 ml) and brine (10 ml), dried over Na₂SO₄ and concentrated to give the title compound as a light yellow solid (610 mg, 2.75 mmol, 47.2% yield). ¹H NMR (400 MHz, DMSO-d₆) δ: 8.38 (d, J=6.8 Hz, 1H), 7.73 (d, J=10.3 Hz, 1H), 7.49 (t, J=71.9 Hz, 1H).

### Intermediate 49

### Methyl 2-bromo-4-(dimethylamino)benzoate

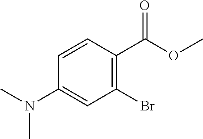

To a stirred solution of methyl 2-bromo-4-fluorobenzoate (5 g, 21.46 mmol) in DMSO (50 mL) were added dimethylamine hydrochloride (2.099 g, 25.7 mmol) and potassium carbonate (6.23 g, 45.1 mmol). The reaction mixture was stirred for 12 h at 70° C. in an autoclave. The reaction mixture was cooled to room temperature and diluted with ice cold water (250 mL) and extracted into DCM (2×100 mL). The combined DCM layers were washed with 10% NaHCO₃ solution (50 mL), dried over Na₂SO₄ and concentrated under reduced pressure to afford the title compound as a pale yellow solid (3.2 g, 11.83 mmol, 55.1%). MS (m/z) 260.0 (M+3H)⁺.

### Intermediate 50

### Methyl 2-chloro-5-fluoro-6-methylnicotinate

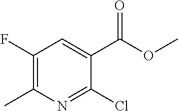

A solution of methyl 2,6-dichloro-5-fluoronicotinate (1.0 g, 4.46 mmol), methylboronic acid (0.267 g, 4.46 mmol) and K₂CO₃ (1.851 g, 13.39 mmol) in 1,4-dioxane (10 mL) and water (10.00 mL) was purged with nitrogen for 15 min before PdCl₂(dppf-CH₂Cl₂ adduct (0.365 g, 0.446 mmol) was added under nitrogen. The resulting reaction mixture was stirred at 100° C. for 16 h. The reaction mixture was cooled to room temperature and filtered through a Celite bed and the Celite bed was washed with ethyl acetate (50 mL). The combined organic layers were concentrated under reduced pressure to afford the title compound as a thick red gum (1.4 g crude). No further purification was performed. GCMS (m/z) 203.1 (M)⁺.

### Intermediate 51

### Methyl 2,5-dichloro-6-methylnicotinate

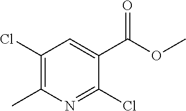

A solution of methyl 2,5,6-trichloronicotinate (3.3 g, 13.72 mmol), methylboronic acid (0.411 g, 6.86 mmol), Tricyclohexylphosphine tetrafluoroborate (1.512 g, 4.12 mmol) and tripotassium phosphate (8.74 g, 41.2 mmol) in toluene (50 mL) and water (12.50 mL) in a tensile seal tube was purged with nitrogen for 15 min before Pd(OAc)₂ (0.431 g, 1.921 mmol) was added. The reaction was purged with nitrogen for 15 min and then stirred at 100° C. for 16 h. The reaction mixture was cooled to room temperature and filtered through a Celite bed and the Celite bed was washed with ethyl acetate (50 mL). The combined organic layers were concentrated under reduced pressure. The crude product was purified by column chromatography (Isolera, 100 g column, 0-20% EtOAc/petroleum ether over 1 h) to give the title compound as a pale yellow gum (2.6 g, 5.46 mmol, 39.8% yield). GCMS (m/z) 219.1 (M+H)⁺.

### Intermediate 52

### Methyl 6-bromo-3-chloro-2-methylbenzoate

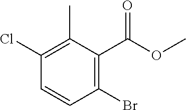

### Step 1: Methyl 3-amino-6-bromo-2-methylbenzoate

To a stirred solution of methyl 3-amino-2-methylbenzoate (5.0 g, 30.3 mmol) in acetic acid (100 mL) and methanol (200 mL) under nitrogen at 0° C., Br₂ (1.559 mL, 30.3 mmol) was added dropwise over 1 min. The resulting reaction mixture was stirred at 0° C. for 15 min and then concentrated under reduced pressure to a brown liquid residue. The residue was dissolved in DCM (200 mL), washed with saturated NaHCO₃ solution (100 mL) and brine (100 mL), dried over Na₂SO₄ and evaporated in vacuo. The crude product was purified by column chromatography (Isolera, 100 g SNAP column, 0-50% EtOAc/petroleum ether over 1 hour) to give the title compound as an orange gum (2.6 g, 10.52 mmol, 34.8% yield). MS (m/z) 243.0 (M)⁺.

### Step 2: Methyl 6-bromo-3-chloro-2-methylbenzoate

To stirred solution of copper(II) chloride (0.220 g, 1.639 mmol) and t-butyl nitrite (0.288 mL, 2.458 mmol) in acetonitrile (5 mL) under nitrogen at 0° C., a solution of methyl 3-amino-6-bromo-2-methylbenzoate (0.2 g, 0.819 mmol) in acetonitrile (1 mL) was added dropwise. The resulting reaction mixture was slowly warmed to room temperature and stirred for 2 hours. Upon completion, the reaction mixture was quenched with water (10 mL) and extracted with EtOAc (2×15 mL). The combined organic phases were washed with brine (10 mL), dried over Na₂SO₄ and evaporated in vacuo. The crude product was purified by column chromatography (Isolera, 5 g SNAP column, 0-15% EtOAc/petroleum ether over 30 min) to give the title compound as an orange gum (75 mg, 0.271 mmol, 33.1% yield). GCMS (m/z) 262.0 (M)⁺.

### Intermediate 53

### Ethyl 2-amino-5-bromo-4-(trifluoromethoxy)benzoate

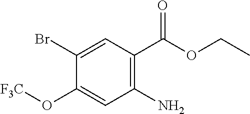 [SMILES: CCOC(=O)c1cc(Br)c(OC(F)(F)F)cc1N]

### Step 1: tert-Butyl (2-bromo-5-(trifluoromethoxy)phenyl)(tert-butoxycarbonyl)carbamate

To a solution of 2-bromo-5-(trifluoromethoxy)aniline (5 g, 19.53 mmol) and DMAP (0.239 g, 1.953 mmol) in THF (100 mL) stirred under nitrogen at RT, Boc-anhydride (13.60 mL, 58.6 mmol) was added dropwise over 5 min. The reaction mixture was refluxed at 90° C. for 4 h. The reaction was concentrated and purified by column chromatography (Isolera, 100 g SNAP column, 2-3% EtOAc/petroleum ether) to give the desired product as an off-white solid (6 g, 12.89 mmol, 66.0% yield). ¹H NMR (400 MHz, DMSO-d₆) δ: 7.85 (d, J=8.8 Hz, 1H), 7.67 (m, 1H), 7.38-7.35 (m, 1H), 1.34 (s, 18H).

### Step 2: Ethyl 2-((tert-butoxycarbonyl)amino)-4-(trifluoromethoxy)benzoate

A solution of tert-butyl (2-bromo-5-(trifluoromethoxy)phenyl)(tert-butoxycarbonyl)carbamate (6 g, 13.15 mmol) and triethylamine (3.99 g, 39.5 mmol) in ethanol (50 mL) was purged with nitrogen for 15 min in a stainless steel autoclave. PdCl₂(dppf-CH₂Cl₂ adduct (1.074 g, 1.315 mmol) was added under nitrogen and the resulting reaction mixture was stirred at 120° C. under CO gas (50 psi) for 16 h. The reaction mixture was cooled to room temperature and concentrated under reduced pressure. The crude product was purified by column chromatography (Isolera, 100 g column, 0-2% EtOAc/petroleum ether over 1 h) to give the title compound as a pale yellow oil (1.7 g, 4.53 mmol, 34.4% yield). MS (m/z) 250 (M-99H)⁺

### Step 3: Ethyl 2-amino-5-bromo-4-(trifluoromethoxy)benzoate

To a solution of ethyl 2-((tert-butoxycarbonyl)amino)-4-(trifluoromethoxy)benzoate (1.5 g, 4.29 mmol) in DMF (10 mL) stirred under nitrogen at RT was added a solution of N-bromosuccinimide (764 mg, 4.29 mmol) in DMF (3 mL) dropwise over 1 min. The reaction mixture was stirred at 100° C. for 16 hr. Ice water (50 mL) was added and the reaction mixture was extracted with EtOAc (2×70 mL). The combined organic phases were washed with brine (20 mL), dried over Na₂SO₄, filtered and evaporated under vacuo. The crude product was purified by column chromatography (Isolera, 50 g SNAP column, 4% EtOAc/petroleum ether) to give the title compound as an off-white solid (560 mg, 1.417 mmol, 33.0%). GCMS (m/z) 327.2 (M+H)⁺/329.2 (M+H)⁺.

### Intermediate 54

### Methyl 5-bromo-4-fluoro-2-nitrobenzoate

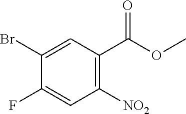

To a stirred solution of m-CPBA (1.670 g, 9.68 mmol) in DCE (60 mL), methyl 2-amino-5-bromo-4-fluorobenzoate (0.6 g, 2.419 mmol) was added at RT and the reaction mixture was heated at 90° C. for 16 h. The reaction mixture was cooled to RT and combined with the material that was obtained from two separate reactions carried out on 600 mg scale each (bromide). The mixture was slowly quenched with saturated sodium thiosulphate solution (100 ml) and extracted with DCM (200 ml). The organic layer was washed with 10% sodium bicarbonate (100 ml) and brine (50 ml), dried over Na₂SO₄ and concentrated under reduced pressure. The crude product was purified by column chromatography (Isolera, 25 g SNAP column, 0-1% EtOAc/petroleum ether) to give the title compound as an off-white solid (1.2 g). GCMS (m/z)=277 (M)⁺/279 (M+H)⁺

### Intermediate 55

### Methyl 4-fluoro-2-nitro-5-(trifluoromethyl)benzoate

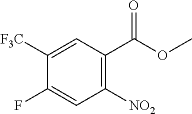 [SMILES: COC(=O)c1cc(C(F)(F)F)c(F)cc1[N+](=O)[O-]]

To a stirred solution of methyl 5-bromo-4-fluoro-2-nitrobenzoate (1.2 g, 4.32 mmol) in DMF (2 mL), methyl 2,2-difluoro-2-(fluorosulfonyl)acetate (7.46 g, 38.8 mmol) and copper(I) iodide (0.740 g, 3.88 mmol) were added and the reaction mixture was heated at 90° C. for 16 h. The reaction mixture was cooled to 30° C., diluted with ethyl acetate (200 mL) and filtered through a Celite pad. The filtrate was washed with water (30 mL) and brine (30 mL), dried over Na₂SO₄, filtered and evaporated in vacuo. The crude product was purified by column chromatography (Isolera, 25 g SNAP column, 0-1% EtOAc/petroleum ether) to give the title compound as an off-white solid (550 mg, 1.977 mmol, 45.8% yield). GCMS (m/z) 266.9 (M)⁺.

Intermediate 56 was prepared from the indicated aryl bromide by methods analogous to those described for Intermediate 55

| Int. | Name | Structure | Characterization | Aryl Bromide |
|---|---|---|---|---|
| 56 | methyl 5-fluoro-2- nitro-4- (trifluoromethyl) benzoate | 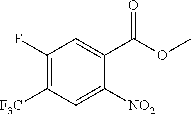 [SMILES: COC(=O)c1cc(F)c(C(F)(F)F)cc1[N+](=O)[O-]] | GCMS (m/z) 267 (M)⁺ | methyl 4- bromo-5- fluoro-2- nitrobenzoate |

### Intermediate 57

### Methyl 2-amino-4-fluoro-5-(trifluoromethyl)benzoate

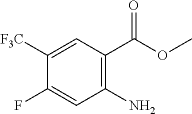 [SMILES: COC(=O)c1cc(C(F)(F)F)c(F)cc1N]

To a stirred solution of methyl 4-fluoro-2-nitro-5-(trifluoromethyl)benzoate (550 mg, 2.059 mmol) in ethanol (18 mL), ammonium chloride (551 mg, 10.29 mmol), water (6 mL) and iron (690 mg, 12.35 mmol) were added at RT and the reaction mixture was stirred at 80° C. for 4 h. The reaction mixture was cooled to 30° C., diluted with ethyl acetate (50 mL) and filtered through a Celite pad. The filtrate was washed with water (10 mL) and brine solution (10 mL), dried over Na₂SO₄, filtered and evaporated in vacuo. The crude product was purified by column chromatography (Isolera, 25 g SNAP column, 0-1% of ethyl acetate/petroleum ether) to give the title compound as an off-white solid (300 mg, 1.202 mmol, 58.4% yield). ¹H NMR (400 MHz, DMSO-d₆) δ: 7.99 (d, J=8.4 Hz, 1H), 7.60-7.40 (m, 2H), 6.74 (d, J=13.6 Hz, 1H), 3.82 (s, 3H).

### Intermediate 58

### Methyl 2-amino-5-fluoro-4-(trifluoromethyl)benzoate

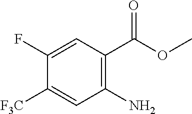 [SMILES: COC(=O)c1cc(F)c(C(F)(F)F)cc1N]

To a stirred solution of methyl 5-fluoro-2-nitro-4-(trifluoromethyl)benzoate (1.6 g, 5.99 mmol) in methanol (8 mL), acetic acid (2.70 g, 44.9 mmol), water (8 mL) and iron (1.472 g, 26.4 mmol) were added and the reaction mixture was stirred at 80° C. for 16 h. The reaction mixture was cooled to 30° C., diluted with ethyl acetate (100 mL) and filtered through a Celite pad. The filtrate was washed with water (20 mL) and brine solution (20 mL), dried over Na₂SO₄, filtered and evaporated in vacuo to give the title compound as a yellow solid (1.2 g, 4.60 mmol, 77%). MS (m/z) 238.0 (M+H)⁺.

### Intermediate 59

### Methyl 2-amino-5-fluoro-4-(trifluoromethoxy)benzoate

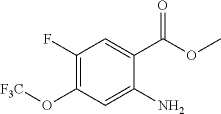 [SMILES: COC(=O)c1cc(F)c(OC(F)(F)F)cc1N]

### Step 1: 2-Bromo-4-fluoro-5-(trifluoromethoxy)aniline

To a solution of 4-fluoro-3-(trifluoromethoxy)aniline (2 g, 10.25 mmol) in acetonitrile (15 mL) at 0° C. under nitrogen was added a solution of NBS (1.916 g, 10.76 mmol) in acetonitrile (10 mL) dropwise over 5 min. The reaction mixture was stirred at rt for 16 h, then concentrated under reduced pressure. The residue was purified by column chromatography (Biotage, 50 g SNAP column, 0-10% EtOAc/hexanes over 30 mins) to give the title compound as a brown liquid (2.4 g, 7.75 mmol, 76% yield). MS (m/z) 272.9 (M+H)⁺.

### Step 2: Methyl 2-amino-5-fluoro-4-(trifluoromethoxy)benzoate

To a solution of 2-bromo-4-fluoro-5-(trifluoromethoxy)aniline (2.4 g, 8.76 mmol) and triethylamine (2.66 g, 26.3 mmol) in methanol (20 mL) under nitrogen was added PdCl₂(Xantphos) (0.331 g, 0.438 mmol). The resulting reaction mixture was stirred at 100° C. under CO gas (50 psi) for 64 h. After 64 h the reaction was concentrated under reduced pressure. The residue was purified by column chromatography (Biotage, 50 g SNAP column, 0-10% EtOAc/hexanes over 30 mins) to give the title compound as a brown solid (750 mg, 2.85 mmol, 32.6% yield). MS (m/z) 254.0 (M+H)⁺.

### Intermediate 60

### 5-Fluoro-6-methoxy-2-methylpyridin-3-amine

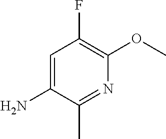

### Step 1: 3-Bromo-5-fluoro-6-methoxy-2-methylpyridine

To a solution of 3-fluoro-2-methoxy-6-methylpyridine (1 g, 7.08 mmol) in DMF (15 mL) stirred under nitrogen at RT was added NBS (1.513 g, 8.50 mmol). The reaction mixture was stirred at RT for 16 h. Reaction mixture was quenched with 50 mL of ice-cold water and extracted with ethyl acetate (50 mL). The organic layer was washed with cold water (2×50 mL), dried over sodium sulphate and concentrated under reduced pressure to give the title compound as colorless oil (1.05 g, 4.74 mmol, 66.9% yield). MS (m/z) 219.0 (M+H)⁺.

### Step 2: N-(5-Fluoro-6-methoxy-2-methylpyridin-3-yl)-1,1-diphenylmethanimine

To a solution of 3-bromo-5-fluoro-6-methoxy-2-methylpyridine (1.2 g, 5.45 mmol), benzophenone imine (1.007 mL, 6.00 mmol) and sodium tert-butoxide (1.310 g, 13.63 mmol) in Toluene (20 mL) under nitrogen were added Pd₂(dba)₃ (0.250 g, 0.273 mmol) and di-tert-butyl(2′,4′,6′-triisopropyl-[1,1′-biphenyl]-2-yl)phosphane (0.232 g, 0.545 mmol). The reaction mixture was stirred at 100° C. for 16 h. The reaction mixture was filtered through Celite and washed with ethyl acetate (2×50 mL). The organic layer was concentrated under reduced pressure. The residue was purified by column chromatography (Biotage, 25 g SNAP column, 0-20% EtOAc/petroleum ether over 30 mins) to give the title compound as a brown gum (1.7 g, 3.36 mmol, 61.7% yield). MS (m/z) 320.8 (M+H)⁺.

### Step 3: 5-fluoro-6-methoxy-2-methylpyridin-3-amine

To N-(5-Fluoro-6-methoxy-2-methylpyridin-3-yl)-1,1-diphenylmethanimine (1.7 g, 3.36 mmol) was added 1.5 N HCl in water (4.49 ml, 6.73 mmol). The resulting suspension was stirred at RT for 16 h. The reaction mixture was diluted with DCM (100 mL). The two layers were separated. The aqueous layer pH was adjusted (pH=8) by using sodium bicarbonate (50 mL) and extracted with DCM (2×50 mL). The combined organics were dried over sodium sulphate and concentrated under the reduced pressure to give the title compound as an off-white solid (500 mg, 2.344 mmol, 69.7% yield). MS (m/z) 157.0 (M+H)⁺.

### Intermediate 61

### Methyl 4-aminofuran-2-carboxylate, Hydrochloride

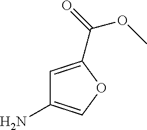

### Step 1: Methyl 4-((diphenylmethylene)amino)furan-2-carboxylate

To a suspension of methyl 4-bromofuran-2-carboxylate (1.8 g, 8.78 mmol), diphenylmethanimine (2.84 g, 10.54 mmol) and Cs₂CO₃ (8.58 g, 26.3 mmol) in 1,4-dioxane (20 mL) under nitrogen were added Pd₂(dba)₃ (0.402 g, 0.439 mmol) and Xantphos (0.508 g, 0.878 mmol). The reaction was stirred at 100° C. for 16 h. The reaction mixture was filtered through Celite and the Celite bed was washed with ethyl acetate (100 mL). The filtered organic layer was concentrated under reduced pressure. The residue was purified by column chromatography (Biotage, 5 g SNAP column, 0-30% EtOAc/petroleum ether over 40 mins) to give the title compound as a brown gum (1.4 g, 4.47 mmol, 50.9% yield). MS (m/z) 305.1 (M+H)⁺.

### Step 2: Methyl 4-aminofuran-2-carboxylate, Hydrochloride

To a solution of methyl 4-((diphenylmethylene)amino)furan-2-carboxylate (1.4 g, 4.59 mmol) in (DCM) (20 mL) under nitrogen at 0° C. was added hydrochloric acid in 1,4 dioxane (4.59 mL, 18.34 mmol) dropwise. The reaction mixture was stirred at RT for 16 h. The reaction mixture was concentrated under reduced pressure. The residue was triturated with diethyl ether (2×10 mL) followed by EtOAc (2×10 mL) to give the title compound as a pale brown solid. MS (m/z) 142.1 (M+H)⁺.

### Intermediate 62

### 1-Bromo-2-(bromomethyl)-4-(trifluoromethyl)benzene

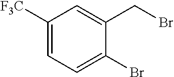 [SMILES: FC(F)(F)c1ccc(Br)c(CBr)c1]

To a solution of 1-bromo-2-methyl-4-(trifluoromethyl)benzene (2 g, 8.37 mmol) and benzoyl peroxide (0.608 g, 2.51 mmol) in Chlorobenzene (10 mL) under nitrogen at RT was added NBS (4.47 g, 25.1 mmol). The reaction mixture was stirred at 80° C. for 16 h then concentrated under reduced pressure. The residue was purified by column chromatography (Biotage, 50 g SNAP column, 5% EtOAc/hexanes over 30 minutes) to give a brown oil (3.1 g, 81%). To a solution of the brown oil (3.1 g, 7.81 mmol) and diethyl phosphite (4.03 mL, 31.2 mmol) in THF (30 mL) under nitrogen at RT was added DIPEA (6.82 mL, 39.1 mmol). The reaction mixture was stirred at RT for 16 h then concentrated under reduced pressure. The residue was purified by column chromatography (Biotage, 25 g SNAP column, 5% EtOAc/hexanes over 30 minutes) to give the title compound as a colorless oil (1.8 g, 3.16 mmol, 40.5% yield). GCMS (m/z) 318.1 (M+H)⁺.

### Intermediate 63

### N-(2-Bromo-5-(trifluoromethyl)benzyl)-6-methoxy-2-methylpyridin-3-amine

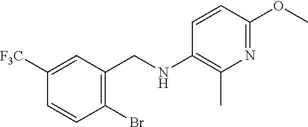 [SMILES: COc1ccc(NCc2cc(C(F)(F)F)ccc2Br)c(C)n1]

To a solution of 1-bromo-2-(bromomethyl)-4-(trifluoromethyl)benzene (1.8 g, 5.66 mmol) and 6-methoxy-2-methylpyridin-3-amine (0.939 g, 6.79 mmol) in acetonitrile (20 mL) under nitrogen at RT was added cesium carbonate (5.53 g, 16.98 mmol). The reaction mixture was stirred at 80° C. for 16 h then concentrated under reduced pressure. To the residue was added EtOAc (50 ml) and water (50 mL). The two layers were separated and the aqueous layer was extracted with EtOAc (2×20 mL). The combined organics were dried over sodium sulphate and concentrated under reduced pressure. The residue was purified by column chromatography (Biotage, 50 g SNAP column, 30% EtOAc/hexanes over 30 minutes) to give the title compound as a brown solid (1.1 g, 2.68 mmol, 47.4% yield). MS (m/z) 377.0 (M+H)⁺.

### Intermediate 64

### 4-Fluoro-2-(2-methoxyethoxy)aniline

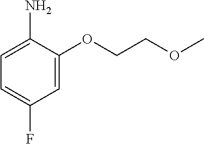

### Step 1: 4-Fluoro-2-(2-methoxyethoxy)-1-nitrobenzene

1-Bromo-2-methoxyethane (1.690 mL, 17.50 mmol) was added dropwise to a stirring mixture of 5-fluoro-2-nitrophenol (2.5 g, 15.91 mmol) and K₂CO₃ (6.60 g, 47.7 mmol) in DMF (35 mL) under N₂ at 0° C. The reaction mixture was stirred at 110° C. for 5 hours. The reaction was cooled, filtered through a pad of Celite and the Celite pad was washed with EtOAc (3×50 mL). The combined filtrates were washed with water (3×50 mL), brine (50 mL), dried over Na₂SO₄, filtered and concentrated to give the title compound as a yellow oil (3.1 g, 13.88 mmol, 87% yield). ¹H NMR (400 MHz, CDCl₃) δ: 7.97 (dd, J=9.1, 6.0 Hz, 1H), 6.85 (dd, J=10.3, 2.5 Hz, 1H), 6.80-6.73 (m, 1H), 4.29-4.23 (m, 2H), 3.86-3.82 (m, 2H), 3.49 (s, 3H).

### Step 2: 4-Fluoro-2-(2-methoxyethoxy)aniline

To a solution of 4-fluoro-2-(2-methoxyethoxy)-1-nitrobenzene (3.1 g, 14.41 mmol) in EtOAc (100 mL) in a 600 mL autoclave vessel, Pd/C (10% wt.) (0.767 g, 0.720 mmol) was added. The reaction mixture was stirred under H₂ (4 kg gas pressure) for 16 hours at room temperature. The reaction was filtered through a pad of Celite and washed with EtOAc (3×50 mL). The filtrate was concentrated to give the title compound as a black oil (2.65 g, 13.95 mmol, 97% yield). MS (m/z) 186.1 (M+H)⁺.

### Intermediate 65

### 4-Fluoro-2-isopropylaniline

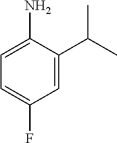

### Step 1: 4-Fluoro-1-nitro-2-(prop-1-en-2-yl)benzene

PdCl₂(dppf-CH₂Cl₂ adduct (0.371 g, 0.455 mmol) was added to a mixture of 2-bromo-4-fluoro-1-nitrobenzene (2.00 g, 9.09 mmol), 4,4,5,5-tetramethyl-2-(prop-1-en-2-yl)-1,3,2-dioxaborolane (1.833 g, 10.91 mmol) and sodium carbonate (1.156 g, 10.91 mmol) in 1,4-dioxane (32 mL) and water (8.00 mL) under N₂. The reaction was purged with N₂ for 20 minutes and then stirred at 80° C. for 16 hours. The reaction was cooled and filtered through a pad of Celite and the Celite pad was washed with DCM (150 mL). The filtrate was washed with water (3×10 mL), dried over Na₂SO₄, filtered and concentrated. The residue was purified by column chromatography (Biotage, 50 g SNAP column, 2% EtOAc/petroleum ether over 20 minutes) to give the title compound as a yellow oil (1.36 g, 7.44 mmol, 82% yield). MS (m/z) 182.0 (M+H)⁺.

### Step 2: 4-Fluoro-2-isopropylaniline

A mixture of 4-fluoro-1-nitro-2-(prop-1-en-2-yl)benzene (1.36 g, 7.51 mmol) and Pd/C (10% wt) (0.799 g, 0.751 mmol) in EtOAc (20 mL) was stirred under H₂ (1 atm) at RT for 16 hours. The reaction mixture was filtered through a pad of Celite and the pad was washed with EtOAc (100 mL). The filtrate was concentrated in vacuo and purified by column chromatography (Biotage, 25 g SNAP column, 7% EtOAc/petroleum ether over 30 minutes) to give the title compound as a colorless oil (980 mg, 6.22 mmol, 83% yield). MS (m/z) 154.2 (M+H)⁺.

### Intermediate 66

### 2-Ethyl-4-fluoroaniline

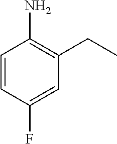

### Step 1: 2-Ethyl-4-fluoro-1-nitrobenzene

To a 250 mL single neck round bottom flask were added 2-bromo-4-fluoro-1-nitrobenzene (8 g, 36.4 mmol), ethylboronic acid (2.96 g, 40.0 mmol) and potassium carbonate (15.08 g, 109 mmol) followed by 1,4-dioxane (80 mL) and water (15.00 mL). The reaction mixture was purged with N₂ for 20 minutes before PdCl₂(dppf-CH₂Cl₂ adduct (1.485 g, 1.818 mmol) was added. After stirring at 100° C. for 16 hours, the reaction was cooled to RT and filtered through a pad of Celite and washed with DCM (300 mL). The filtrate was concentrated under reduced pressure and the residue was purified by column chromatography (Biotage, 100 g SNAP column, 7% EtOAc/petroleum ether over 40 minutes) to give the title compound as a pale-yellow oil (3.4 g, 20.10 mmol, 55.3% yield). ¹H NMR (400 MHz, CDCl₃) δ: 8.01 (dd, J=9.0, 5.5 Hz, 1H), 7.12-7.00 (m, 2H), 2.98 (q, J=7.5 Hz, 2H), 1.32 ppm (t, J=7.5 Hz, 3H).

### Step 2: 2-Ethyl-4-fluoroaniline

To a stirring solution of 2-ethyl-4-fluoro-1-nitrobenzene (3.4 g, 20.10 mmol) in THF (10 mL), ethanol (10.00 mL) and water (2.00 mL) were added Iron powder (5.64 g, 101 mmol) and Ammonium chloride (1.613 g, 30.1 mmol) at 25° C. The resulting reaction mixture was slowly heated to 90° C. and stirred for 3 hours. The reaction was cooled and filtered through a pad of Celite and the Celite pad was washed with DCM (100 mL). The filtrate was concentrated under the reduced pressure to a brown gum residue which was diluted with DCM (20 mL) and washed with saturated NaHCO₃ (20 mL). The organic phase was dried over Na₂SO₄ filtered and concentrated in vacuo. The residue was purified by column chromatography (Biotage, 100 g SNAP column, 15-20% EtOAc/petroleum ether over 30 mins) to give the title compound as a brown oil (1.6 g, 10.96 mmol, 54.5% yield). MS (m/z) 140.2 (M+H)⁺.

### Intermediate 67

### 2-Cyclopropyl-4-fluoroaniline

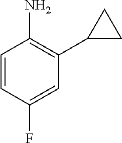 [SMILES: Nc1ccc(F)cc1C1CC1]

### Step 1: 2-Cyclopropyl-4-fluoro-1-nitrobenzene

PdCl₂(dppf-CH₂Cl₂ adduct (0.453 g, 0.555 mmol) was added to a mixture of 2-bromo-4-fluoro-1-nitrobenzene (0.61 g, 2.77 mmol), 2-cyclopropyl-4,4,5,5-tetramethyl-1,3,2-dioxaborolane (0.556 mL, 3.05 mmol) and cesium carbonate (1.807 g, 5.55 mmol) in 1,4-dioxane (19 mL) and water (3.80 mL) under N₂. The reaction was purged with N₂ for 20 minutes and then heated under microwave at 80° C. for 1.5 hours. The reaction was cooled to RT, filtered through a pad of Celite and washed with EtOAc (150 mL). The filtrate was washed with water (150 mL) and brine (100 mL), dried over Na₂SO₄, filtered and concentrated. The residue was purified by column chromatography (Isco, 40 g RediSep Rf Gold high performance flash columns, 0-15% EtOAc/heptane over 30 mins) to give the title compound as a yellow solid (0.39 g, 2.153 mmol, 78% yield). ¹H NMR (400 MHz, DMSO-d₆) δ: 7.99 (dd, J=5.1, 9.0 Hz, 1H), 7.26 (ddd, J=2.7, 7.8, 9.0 Hz, 1H), 7.11 (dd, J=2.4, 10.3 Hz, 1H), 2.32-2.25 (m, 1H), 1.07-1.02 (m, 2H), 0.86-0.82 (m, 2H).

### Step 2: 2-Cyclopropyl-4-fluoroaniline

A mixture of 2-cyclopropyl-4-fluoro-1-nitrobenzene (0.532 g, 2.94 mmol) and Pd/C (10% wt) (0.313 g, 0.294 mmol) in methanol (10 mL) was stirred under H₂ (1 atm) at RT for 16 hours. The reaction mixture was filtered through a pad of Celite and the pad was washed with EtOAc (100 mL). The filtrate was concentrated in vacuo and purified by column chromatography (Isco, 24 g RediSep Rf Gold high performance flash columns, 0-30% EtOAc/heptane over 30 mins) to give the title compound as a yellow oil (253 mg, 1.67 mmol, 57% yield). MS (m/z) 152.2 (M+H)⁺.

### Intermediate 68

### 4-Fluoro-3-methoxy-2-methylaniline

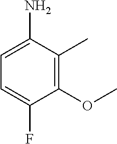

### Step 1: 3-Bromo-4-fluoro-2-methylaniline

To a solution of 2-bromo-1-fluoro-3-methyl-4-nitrobenzene (2.0 g, 8.55 mmol) in methanol (60 mL) stirred under nitrogen at room temperature was added a solution of ammonium chloride (2.286 g, 42.7 mmol) dissolved in water (40 mL), followed by the addition of iron (2.386 g, 42.7 mmol) in one lot. The reaction mixture was stirred at 80° C. for 22 hours. The reaction mixture was cooled to room temperature and the solvents were removed under reduced pressure. The crude material was diluted with water (100 mL) and DCM (100 mL). Layers were separated and the aqueous phase was extracted with DCM (2×50 mL). The combined organic phases were washed with saturated sodium chloride solution (50 mL) and water (50 mL), dried over Na₂SO₄ and evaporated in vacuo to give the crude compound. The residue was purified by column chromatography (Biotage, 50 g column, 0-40% EtOAc/petroleum ether over 45 mins) to give the title compound as a brown solid (1.26 g, 5.82 mmol, 68% yield). MS (m/z) 203.9 (M+H)⁺.

### Step 2: 4-Fluoro-2-methyl-3-(4,4,5,5-tetramethyl-1,3,2-dioxaborolan-2-yl)aniline

To a solution of 3-bromo-4-fluoro-2-methylaniline (1.25 g, 6.13 mmol) in 1,4-dioxane (30 mL) were added bis(pinacolato)diboron (2.334 g, 9.19 mmol) and potassium acetate (1.804 g, 18.38 mmol) and stirred under nitrogen at room temperature. Reaction mixture was degassed with nitrogen for 20 minutes, followed by the addition of PdCl₂(dppf-CH₂Cl₂adduct (0.750 g, 0.919 mmol) at room temperature. The reaction mixture was stirred at 100° C. for 20 hours. The reaction mixture was cooled to room temperature and concentrated under vacuum. The crude material was dissolved in water (100 mL) and ethyl acetate (80 mL). The layers were separated and the aqueous layer was extracted with ethyl acetate (2×60 mL). The combined organic phases were washed with saturated sodium chloride solution (60 mL) and water (50 mL). The organic layer was dried over Na₂SO₄ and evaporated in vacuo to give the crude compound. The residue was purified by column chromatography (Biotage, 50 g column, 0-50% EtOAc/petroleum ether over 60 mins) to give the title compound. (1.06 g, 4.19 mmol, 69% yield). MS (m/z) 252.1 (M+H)⁺.

### Step 3: 3-Amino-6-fluoro-2-methylphenol

To a solution of 4-fluoro-2-methyl-3-(4,4,5,5-tetramethyl-1,3,2-dioxaborolan-2-yl)aniline (1.05 g, 4.18 mmol) in THF (20 mL) being stirred under nitrogen at room temperature were added sodium perborate tetrahydrate (1.930 g, 12.54 mmol) and water (10 mL) in one charge. The reaction mixture was stirred at RT for 5 hr and then concentrated under vacuum. The crude material was dissolved in water (70 mL) and EtOAc (50 mL). The layers were separated and the aqueous layer was extracted with ethyl acetate (2×40 mL). The combined organic phases were washed with a saturated sodium chloride solution (40 mL) and water (40 mL). The organic layer was dried over Na₂SO₄ and evaporated in vacuo to give the crude compound. The residue was purified by column chromatography (Biotage, 25 g column, 0-50% EtOAc/petroleum ether over 60 mins) to give the title compound. (388 mg, 2.73 mmol, 65% yield). MS (m/z) 142.1 (M+H)⁺.

### Step 4: 4-Fluoro-3-methoxy-2-methylaniline

To a solution of 3-amino-6-fluoro-2-methylphenol (330 mg, 2.321 mmol) in Dimethyl Carbonate (15 mL) was added DBU (0.420 mL, 2.79 mmol) and the reaction was stirred under nitrogen at 115° C. for 16 hours. Reaction mixture was cooled to room temperature and concentrated under vacuum. Crude material was dissolved in water (40 mL) and ethyl acetate (50 mL). The layers were separated and the aqueous layer was extracted with ethyl acetate (2×50 mL). The combined organic phases were dried over Na₂SO₄ and evaporated in vacuo to give the crude compound. The residue was purified by column chromatography (Biotage, 25 g column, 0-50% EtOAc/petroleum ether over 60 mins) to give the title compound. (277 mg, 1.75 mmol, 75% yield). MS (m/z) 156.1 (M+H)⁺.

### Intermediate 69

### 2-(tert-Butyl)-4-fluoroaniline

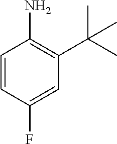

### Step 1: N-(2-(tert-Butyl)phenyl)acetamide

To a stirring solution of 2-(tert-butyl)aniline (5 g, 33.5 mmol) in THF (350 mL) were added TEA (15.43 mL, 111 mmol) and acetyl chloride (2.63 mL, 36.8 mmol) at 30° C. The reaction mixture was stirred at 30° C. for one hour. Upon completion, the reaction mixture was quenched with water (200 mL) and extracted with EtOAc (2×200 mL). The combined organic phases were washed with water (100 mL) and brine (50 mL), dried over Na₂SO₄ and evaporated in vacuo to give the title compound as a brown solid. No purification was carried out on this material. MS (m/z) 192.2 (M+H)⁺.

### Step 2: N-(2-(tert-Butyl)-4-fluorophenyl)acetamide

To a stirred solution of N-(2-(tert-butyl)phenyl)acetamide (10.0 g, 52.3 mmol) and HF-pyridine (70%) (30.2 mL, 209 mmol) in DCM (100 mL) under nitrogen at 0° C., a solution of iodobenzene diacetate (25.3 g, 78 mmol) dissolved in (DCM) (100 mL) was added dropwise. The reaction mixture was stirred at room temperature for 16 hours. The reaction mass was cooled to 0° C. and quenched with TEA (10 mL). The reaction mass was concentrated under reduced pressure to a brown residue. The crude product was purified by column chromatography (Isolera, 340 g SNAP column, 0-60% EtOAc/petroleum ether over 2 hours) to give the title compound as an off-white solid (4.5 g, 21.24 mmol, 40.6% yield). MS (m/z) 210.2 (M+H)⁺.

### Step 3: 2-(tert-Butyl)-4-fluoroaniline

To a stirred solution of N-(2-(tert-butyl)-4-fluorophenyl)acetamide (4.5 g, 21.50 mmol) in ethanol (200 mL) under nitrogen at 0° C., HCl (concentrated, 12.0 M) (35.8 mL, 430 mmol) was added dropwise. The reaction mixture was stirred at 100° C. for 24 hours, cooled to room temperature and concentrated. The resultant brown residue was dissolved in EtOAc (200 mL) and washed with saturated sodium bicarbonate solution (100 mL). The organic phase was washed with brine (50 mL), dried over Na₂SO₄ and evaporated in vacuo to give the crude product as a brown liquid. The crude product was purified by column chromatography (Isolera, 50 g SNAP column, 0-50% EtOAc/petroleum ether over 40 mins) to give the title compound as an orange liquid (1.95 g, 10.95 mmol, 50.9% yield). MS (m/z) 168.2 (M+H)⁺.

### Intermediate 70

### 4-Aminopicolinamide

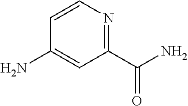

To a solution of 4-aminopicolinonitrile (800 mg, 6.72 mmol) in ethanol (24 mL), that was stirred under nitrogen at room temperature, was added a solution of KOH (942 mg, 16.79 mmol) in water (6 mL). The reaction mixture was stirred at 80° C. for 3.5 hour. The reaction mixture was concentrated under vacuum. The crude material was dissolved in water (50 mL) and 10% MeOH in DCM (60 mL). The layers were separated and the aqueous layer was extracted with 10% of MeOH in DCM (2×100 mL). The combine organic phases were washed with saturated sodium chloride solution (50 mL) and water (50 mL), dried over Na₂SO₄ and evaporated in vacuo to give the crude product. The residue was purified by column chromatography (Biotage, 50 g column, 0-20% MeOH/DCM over 60 mins) to give the title compound. (165 mg, 1.19 mmol, 18% yield). MS (m/z) 138 (M+H)⁺.

### Intermediate 71

### 4-Chloro-2-methoxypyrimidin-5-amine

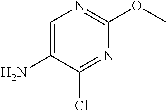

To a solution of 4-chloro-2-methoxy-5-nitropyrimidine (1 g, 5.28 mmol) in ethanol (10 mL) and acetic acid (5 mL) was added iron (1.768 g, 31.7 mmol). After stirring at 90° C. for 20 min, the reaction mixture was filtered through Celite washing with EtOAc (3×15 ml). The filtrate was concentrated to give the title compound as an off-white solid (0.23 g, 1.009 mmol, 19.13% yield). MS (m/z) 160.1 (M+H)⁺.

### Intermediate 72

### 2-Methoxy-6-((tetrahydro-2H-pyran-2-yl)oxy)pyridin-3-amine

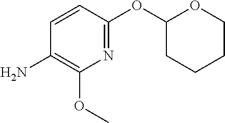 [SMILES: COc1nc(OC2CCCCO2)ccc1N]

Step 1: 2-Methoxy-3-nitro-6-((tetrahydro-2H-pyran-2-yl)oxy)pyridine

To a mixture of 6-chloro-2-methoxy-3-nitropyridine (773 mg, 4.10 mmol) and sodium hydride (60% wt in mineral oil) (295 mg, 7.38 mmol) in THF (16.0 mL) was slowly added tetrahydro-2H-pyran-2-ol (628 mg, 6.15 mmol) at 0° C. The reaction was stirred at 0° C. for 1 h and then warmed to room temperature. After stirring for 1.5 h, the reaction was quenched with sat. NH₄Cl aqueous solution and extracted with EtOAc (3×). The combined organic layers were washed with brine, dried over MgSO₄, filtered and concentrated to give the title compound as a pale yellow solid (406 mg, 1.597 mmol, 39% yield). ¹H NMR (400 MHz, CDCl₃) δ: 8.38 (d, J=8.8 Hz, 1H), 6.45 (d, J=8.8 Hz, 1H), 6.30 (t, J=2.9 Hz, 1H), 4.09 (s, 3H), 3.96-3.90 (m, 1H), 3.72-3.66 (m, 1H), 2.04-1.88 (m, 3H), 1.79-1.62 (m, 3H).

### Step 2: 2-Methoxy-6-((tetrahydro-2H-pyran-2-yl)oxy)pyridin-3-amine

To a solution of 2-methoxy-3-nitro-6-((tetrahydro-2H-pyran-2-yl)oxy)pyridine (396 mg, 1.558 mmol) in THF (10 mL), Pd/C (10% wt) (0.166 g, 0.156 mmol) was added. The reaction mixture was stirred under H₂ (1 atm) for 3 days at room temperature. The reaction was filtered through a pad of Celite and washed with acetonitrile. The filtrate was concentrated under reduced pressure to give the title compound as a black oil (310 mg, 1.381 mmol, 89% yield). MS (m/z) 225.3 (M+H)⁺.

### Intermediate 73

### (5S,6R)-5-amino-6-methylpiperidin-2-one

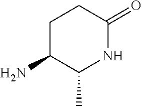

### Step 1: (5S,6R)-6-methyl-5-nitropiperidin-2-one

To a solution of methyl 4-nitrobutanoate (5 g, 34.0 mmol) in ethanol (60 mL) were added acetaldehyde (1.919 mL, 34.0 mmol) and ammonium acetate (5.24 g, 68.0 mmol) and the reaction solution was refluxed for 18 hr. Solvent was removed under reduced pressure. MeOH (50 ml) was added and concentrated repeatedly three times. To the semisolid residue EtOH (˜20 ml) was added and the solid remained was filtered and washed with more EtOH (10 ml). The solid was dried in the oven for 1 h to afford the title compound (1.8 g, 11.38 mmol, 33.5% yield). MS (m/z) 159.0 (M+H)⁺.

### Step 2: 6-Methyl-5-nitropiperidin-2-one

A solution of 6-methyl-5-nitropiperidin-2-one (50 mg, 0.316 mmol) in ethanol (10 mL) was added to Pd/C (5%, 20 mg) and the resulting solution was hydrogenated for 16 hr. The catalyst was filtered off under nitrogen and the filtrate was concentrated under reduced pressure to give the title compound (30 mg, 0.154 mmol). Product was carried on directly to the next reaction.

### Intermediate 74

### 2-Bromo-6-methoxy-4-methylpyridin-3-amine

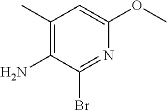

6-Methoxy-4-methylpyridin-3-amine (2.073 g, 15 mmol) and sodium acetate (1.231 g, 15.00 mmol) were added to acetic acid (15 mL) and the reaction was stirred for 30 min. Bromine (0.773 mL, 15.00 mmol) was then added to the reaction and the reaction was stirred at room temperature for 2 hr. The reaction mixture was cooled by ice water bath and neutralized by addition of 5N NaOH solution. The aqueous layer was extracted with EtOAc (3×60 mL). The combined organic layers were washed with water (40 mL) and brine (40 mL), dried over Na₂SO₄ and concentrated. The crude product was purified by column chromatography (Isco, 120 g silica column, EtOAc/heptane 0%-30%) to give the title compound (1.952 g, 8.99 mmol, 60.0% yield). MS (m/z) 217.1 (M+H)⁺/219.1 (M+3H)⁺

### Intermediate 75

### 2-Methoxy-4,6-dimethylpyrimidin-5-amine

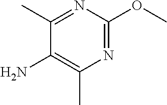

To a stirred solution of 2-methoxy-4,6-dimethyl-5-nitropyrimidine (900 mg, 4.91 mmol) in THF (25 mL) under nitrogen at RT, Pd/C (10% wt) (105 mg, 0.098 mmol) was added portionwise. The reaction mixture was stirred at room temperature under hydrogen balloon pressure for 16 hours. Upon completion, the reaction mass was filtered through a Celite pad washing with THF (2×75 mL). The filtrate was evaporated in vacuo to give the title compound as a white solid (700 mg, 4.39 mmol, 89% yield). MS (m/z) 154.1 (M+H)⁺.

### Intermediate 76

### 4-chloro-6-methoxy-2-methylpyridin-3-amine

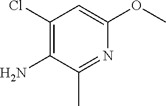

### Step 1: 4-chloro-6-methyl-5-nitropyridin-2(1H)-one

In a 500 mL three neck round bottom flask fitted with a Dewar condenser, ammonia (200 mL) was condensed into THF (100 mL) at −78° C. Potassium tert-butoxide (6.99 g, 62.3 mmol) was added and the resulting reaction mixture was allowed to warm to −35° C. In a separate flask containing a stirring solution of 4-chloro-2-methyl-3-nitropyridine (4.3 g, 24.92 mmol) in THF (50 mL), tert-butyl hydroperoxide (5.5 M in decane) (4.76 mL, 26.2 mmol) was added at 0° C. The resulting solution was added to the reaction mixture above at −35° C. (color of the reaction mixture was changed from light brown to dark brown). After stirring for 1.5 hours at −35° C. the reaction mixture was quenched with saturated ammonium chloride solution (100 mL) and the reaction mixture was allowed warm to RT and stirred for 16 hours. Upon completion, the reaction mixture was concentrated under reduced pressure. The resultant brown residue was diluted with water (100 mL) and extracted with EtOAc (2×100 mL). The combined organic phases were washed with brine (100 mL), dried over Na₂SO₄ and evaporated in vacuo to give the title compound as a brown solid (2.8 g, 12.45 mmol, 50.0% yield). MS (m/z) 187.0 (M−H)⁻.

### Step 2: 4-Chloro-6-methoxy-2-methyl-3-nitropyridine

To a stirring solution of 4-chloro-6-methyl-5-nitropyridin-2(1H)-one (2.5 g, 13.26 mmol) and silver carbonate (5.48 g, 19.89 mmol) in THF (50 mL) under nitrogen at 0° C., methyl iodide (4.14 mL, 66.3 mmol) was added. The reaction mixture was stirred at 30° C. for 16 hours. Upon completion, the reaction mass was filtered through a Celite pad and the Celite pad was washed with EtOAc (2×50 mL). The filtrate was concentrated under reduced pressure to a brown gum. The crude product was purified by column chromatography (Isolera, 50 g SNAP column, 0-50% EtOAc/petroleum ether) to give the title compound as a yellow solid (0.85 g, 4.16 mmol, 31.4% yield). MS (m/z) 203.0 (M+H)⁺.

### Step 3: 4-Chloro-6-methoxy-2-methylpyridin-3-amine

To a stirring solution of 4-chloro-6-methoxy-2-methyl-3-nitropyridine (0.65 g, 3.21 mmol) in ethanol (30 mL), iron (1.075 g, 19.25 mmol) was added followed by a solution of ammonium chloride (1.030 g, 19.25 mmol) in water (6 mL) under nitrogen at RT. The reaction mixture was stirred at 80° C. for 2 hours, cooled to room temperature and filtered through a Celite pad washing with EtOH (4×50 mL). The filtrate was evaporated in vacuo to give the residue as a yellow solid. The residue was dissolved in water (50 mL) and extracted with DCM (2×50 mL). The combined organic phases were washed with brine (30 mL), dried over Na₂SO₄ and evaporated in vacuo to give the title compound as a yellow gum (520 mg, 2.95 mmol, 92% yield). MS (m/z) 173.2 (M+H)⁺.

### Intermediate 77

### 6-Methoxy-4-methylpyridazin-3-amine

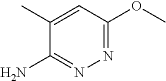

In a microwave reaction vessel, 6-chloro-4-methylpyridazin-3-amine (0.4 g, 2.79 mmol) and sodium methoxide (25% in methanol) (9.56 ml, 41.8 mmol) were charged. The reaction vessel was sealed and heated in Biotage Initiator using initial high to 130° C. for 1 hour. Upon completion, the reaction mass was cooled to room temperature and diluted with DCM (50 mL), washed with water (25 mL) and brine (20 mL), dried over Na₂SO₄ and evaporated in vacuo to give the title compound as a brown solid (245 mg, 1.467 mmol, 52.7% yield). MS (m/z) 140.1 (M+H)⁺.

### Intermediate 78

### Ethyl 2-((4-fluoro-2-methylphenyl)amino)-4-(trifluoromethyl)benzoate

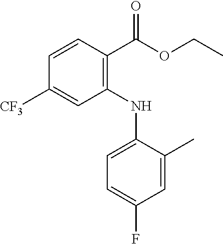 [SMILES: CCOC(=O)c1ccc(C(F)(F)F)cc1Nc1ccc(F)cc1C]

In a sealed tube, a mixture of ethyl 2-bromo-4-(trifluoromethyl)benzoate (3.5 g, 11.78 mmol), 4-fluoro-2-methylaniline (2.212 g, 17.67 mmol), and Cs₂CO₃ (5.76 g, 17.67 mmol) in Toluene (30 mL) was purged with N₂ for 10 minutes before Pd₂(dba)₃ (0.539 g, 0.589 mmol) and BINAP (0.734 g, 1.178 mmol) were added. The reaction was stirred at 100° C. for 16 hours. The reaction was cooled to RT, diluted with EtOAc (50 mL) and washed with water (2×25 mL). The organic layer was washed with brine, dried over Na₂SO₄, filtered and concentrated in vacuo. The residue was purified by column chromatography (Biotage, 50 g SNAP column, 0-15% EtOAc/petroleum ether over 45 minutes) to give the title compound as an orange gum (3.35 g, 9.80 mmol, 83% yield). MS (m/z) 342.0 (M+H)⁺.

Intermediates 79-194 were prepared from the indicated aryl halogen and aniline by methods analogous to those described for Intermediate 78.

| Int. | Name | Structure | Characterization | Aryl halogen | Aniline |
|---|---|---|---|---|---|
| 79 | ethyl 2-((4- fluoro-2,6- dimethyl- phenyl)- amino)-4- (trifluoro- methyl)- benzoate | 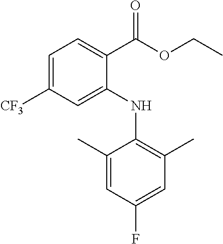 [SMILES: CCOC(=O)c1ccc(C(F)(F)F)cc1Nc1c(C)cc(F)cc1C] | MS (m/z) 355.9 (M + H)⁺ | ethyl 2-bromo- 4-(trifluorometh- yl)benzoate | 4-fluoro- 2,6-dimeth- ylaniline |
| 80 | ethyl 2-((2- chloro-4- fluorophen- yl)amino)- 4-(trifluoro- methyl)- benzoate | 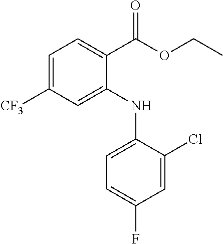 [SMILES: CCOC(=O)c1ccc(C(F)(F)F)cc1Nc1ccc(F)cc1Cl] | MS (m/z) 361.9 (M + H)⁺ | ethyl-2-bromo- 4-(trifluorometh- yl)benzoate | 2-chloro-4- fluoroaniline |
| 81 | ethyl 2-((4- fluoro-2-(2- methoxy- ethoxy)phen- yl)amino)-4- (trifluoro- methyl)- benzoate | 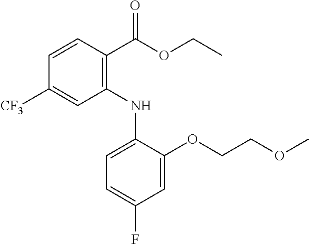 [SMILES: CCOC(=O)c1ccc(C(F)(F)F)cc1Nc1ccc(F)cc1OCCOC] | MS (m/z) 402.0 (M + H)⁺ | ethyl 2-bromo- 4-(trifluorometh- yl)benzoate | 4-fluoro-2- (2-meth- oxyethoxy)- aniline |
| 82 | ethyl 2-((2,4- difluorophen- yl)amino)- 4-(trifluoro- methyl)- benzoate | 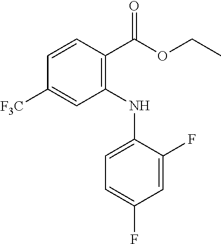 [SMILES: CCOC(=O)c1ccc(C(F)(F)F)cc1Nc1ccc(F)cc1F] | MS (m/z) 346.0 (M + H)⁺ | ethyl 2-bromo- 4-(trifluorometh- yl)benzoate | 2,4-difluoro- aniline |
| 83 | ethyl 2-((2- methyl- pyridin-3- yl)amino)-4- (trifluoro- methyl)- benzoate | 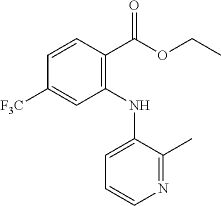 [SMILES: CCOC(=O)c1ccc(C(F)(F)F)cc1Nc1cccnc1C] | MS (m/z) 325.0 (M + H)⁺ | ethyl 2-bromo- 4-(trifluorometh- yl)benzoate | 2-methyl- pyridin-3- amine |
| 84 | ethyl 2-((4- fluoro-2- isopropyl- phenyl)- amino)-4- (trifluoro- methyl)- benzoate | 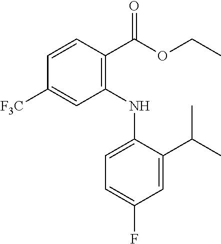 [SMILES: CCOC(=O)c1ccc(C(F)(F)F)cc1Nc1ccc(F)cc1C(C)C] | MS (m/z) 370.2 (M + H)⁺ | ethyl 2-bromo- 4-(trifluorometh- yl)benzoate | 4-fluoro-2- isopropyl- aniline |
| 85 | ethyl 5- chloro-2-((4- fluoro-2- methylphen- yl)amino)- benzoate | 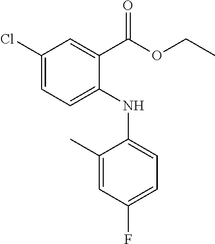 [SMILES: CCOC(=O)c1cc(Cl)ccc1Nc1ccc(F)cc1C] | MS (m/z) 308.0 (M + H)⁺ | ethyl 2-bromo- 5-chlorobenzoate | 4-fluoro-2- methylaniline |
| 86 | methyl 2- ((4-fluoro- phenyl)- amino)- benzoate | 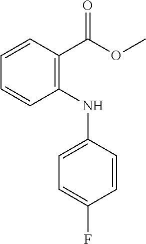 [SMILES: COC(=O)c1ccccc1Nc1ccc(F)cc1] | MS (m/z) 246.0 (M + H)⁺ | methyl 2-bromo- benzoate | 4-fluoro- aniline |
| 87 | methyl 2-(o- tolylamino)- benzoate | 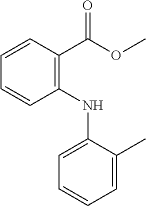 [SMILES: COC(=O)c1ccccc1Nc1ccccc1C] | MS (m/z) 242.0 (M + H)⁺ | methyl 2-bromo- benzoate | o-toluidine |
| 88 | ethyl 2-((4- fluoro-2- methylphen- yl)amino)- 5-methyl- benzoate | 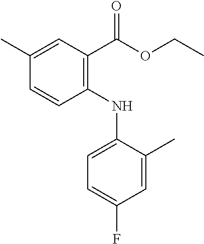 [SMILES: CCOC(=O)c1cc(C)ccc1Nc1ccc(F)cc1C] | MS (m/z) 288.1 (M + H)⁺ | ethyl 2-bromo- 5-methylbenzoate | 4-fluoro-2- methylaniline |
| 89 | methyl 2- ((4-fluoro- methylphen- yl)amino)- benzoate | 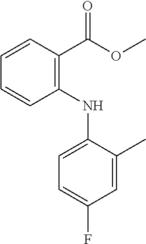 [SMILES: COC(=O)c1ccccc1Nc1ccc(F)cc1C] | MS (m/z) 260.0 (M + H)⁺ | methyl 2-bromo- benzoate | 4-fluoro-2- methylaniline |
| 90 | ethyl 2-((4- fluoro-2- methylphen- yl)amino)- 6-(trifluoro- methyl)- nicotinate | 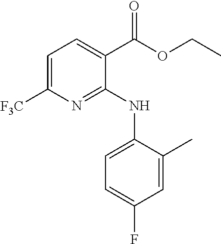 [SMILES: CCOC(=O)c1ccc(C(F)(F)F)nc1Nc1ccc(F)cc1C] | MS (m/z) 343.2 (M + H)⁺ | ethyl 2-bromo- 6-(trifluorometh- yl)nicotinate | 4-fluoro-2- methylaniline |
| 91 | ethyl 2-((4- fluoro-2- methylphen- yl)amino)-5- (trifluoro- methyl)- benzoate | 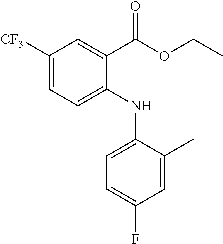 [SMILES: CCOC(=O)c1cc(C(F)(F)F)ccc1Nc1ccc(F)cc1C] | MS (m/z) 341.1 (M + H)⁺ | ethyl 2-bromo- 5-(trifluorometh- yl)benzoate | 4-fluoro-2- methylaniline |
| 92 | ethyl 2-((4- fluoro-4- methoxy- phenyl)- amino)-4- (trifluoro- methyl)- benzoate | 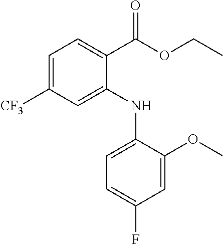 [SMILES: CCOC(=O)c1ccc(C(F)(F)F)cc1Nc1ccc(F)cc1OC] | MS (m/z) 358.0 (M + H)⁺ | ethyl 2-bromo- 4-(trifluorometh- yl)benzoate | 4-fluoro-2- methoxy- aniline |
| 93 | ethyl 2-((4- fluoro-2- methylphen- yl)amino)-6- (trifluoro- methyl)- benzoate | 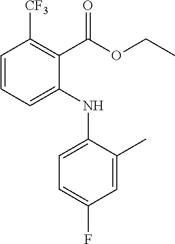 [SMILES: CCOC(=O)c1c(Nc2ccc(F)cc2C)cccc1C(F)(F)F] | MS (m/z) 342.0 (M + H)⁺ | ethyl 2-bromo- 6-(trifluorometh- yl)benzoate | 4-fluoro-2- methylaniline |
| 94 | ethyl 2-((4- fluoro-2- methoxy- phenyl)- amino)-6- (trifluoro- methyl)- benzoate | 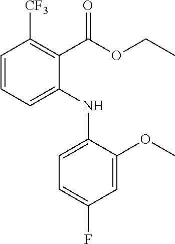 [SMILES: CCOC(=O)c1c(Nc2ccc(F)cc2OC)cccc1C(F)(F)F] | MS (m/z) 358.0 (M + H)⁺ | ethyl 2-bromo- 6-(trifluorometh- yl)benzoate | 4-fluoro-2- methoxy- aniline |
| 95 | methyl 2-((4- fluoro-2- methylphen- yl)amino)-4- (trifluoro- methoxy)- benzoate | 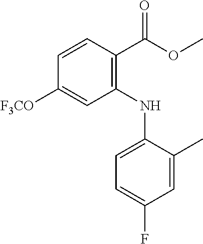 [SMILES: COC(=O)c1ccc(OC(F)(F)F)cc1Nc1ccc(F)cc1C] | MS (m/z) 344.0 (M + H)⁺ | methyl 2-bromo- 4-(trifluorometh- oxy)benzoate | 4-fluoro-2- methylaniline |
| 96 | ethyl 4- chloro-2-((4- fluoro-2- methylphen- yl)amino)- benzoate | 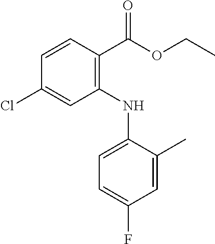 [SMILES: CCOC(=O)c1ccc(Cl)cc1Nc1ccc(F)cc1C] | MS (m/z) 308.0 (M + H)⁺ | ethyl 2-bromo- 4-chlorobenzoate | 4-fluoro-2- methylaniline |
| 97 | methyl 2-((4- fluoro-2- methylphen- yl)amino)-5- (trifluoro- methyl)- benzoate | 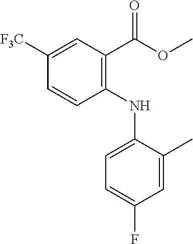 [SMILES: COC(=O)c1cc(C(F)(F)F)ccc1Nc1ccc(F)cc1C] | MS (m/z) 328.0 (M + H)⁺ | methyl 2-bromo- 5-(trifluorometh- yl)benzoate | 4-fluoro-2- methylaniline |

### Intermediate 97

### methyl 2-((4-fluoro-2-methylphenyl)amino)-5-(trifluoromethyl)benzoate

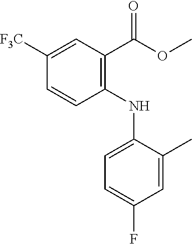 [SMILES: COC(=O)c1cc(C(F)(F)F)ccc1Nc1ccc(F)cc1C]

To a solution of methyl 2-bromo-5-(trifluoromethyl)benzoate (2 g, 7.1 mmol) and 4-fluoro-2-methylaniline (1.06 g, 8.48 mmol) in 1,4-dioxane (20 mL) under nitrogen at room temperature were added Cs₂CO₃ (4.60 g, 14.13 mmol) and BINAP (0.44 g, 0.71 mmol) in one charge. The reaction mixture was purged with nitrogen for 10 min, then Pd₂(dba)₃ (0.324 g, 0.353 mmol) was added into reaction mixture. The reaction mixture was stirred at 100° C. for 16 h. The reaction mixture was cooled to room temperature and filtered through celite pad and the filtrate was concentrated onto SiO₂. Purification by flash chromatography on SiO₂ (25 g) with 0→30% EtOAc in petroleum ether as eluant afforded the title compound as a colorless solid (2.3 g, 7.0 mmol, 99% yield). MS (m/z) 328.0 (M+H)⁺.

| | | | | | |
|---|---|---|---|---|---|
| 98 | methyl 4,5- difluoro-2- ((4-fluoro- 2-methyl- phenyl)- amino)- benzoate | 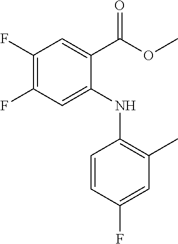 [SMILES: COC(=O)c1cc(F)c(F)cc1Nc1ccc(F)cc1C] | MS (m/z) 296.2 (M + H)⁺ | methyl 2-bromo- 4,5-difluoro- benzoate | 4-fluoro-2- methylaniline |
| 99 | methyl 4,5- difluoro-2- ((4-fluoro-2- isopropyl- phenyl)amino)- benzoate | 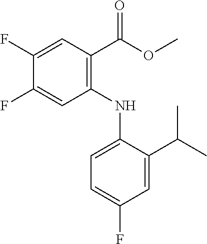 [SMILES: COC(=O)c1cc(F)c(F)cc1Nc1ccc(F)cc1C(C)C] | MS (m/z) 324.2 (M + H)⁺ | methyl 2-bromo- 4,5-difluoro- benzoate | 4-fluoro-2- isopropyl- aniline |
| 100 | methyl 4- cyano-2-((4- fluoro-2- isopropylphen- yl)amino)- benzoate | 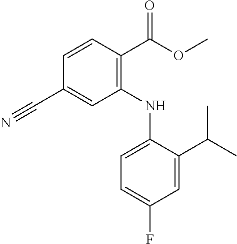 [SMILES: COC(=O)c1ccc(C#N)cc1Nc1ccc(F)cc1C(C)C] | MS (m/z) 313.2 (M + H)⁺ | methyl 2-bromo- 4-cyanobenzoate | 4-fluoro-2- isopropyl- aniline |
| 101 | methyl 2-((2- ethyl-4- fluorophenyl)- amino)-5- (trifluorometh- yl)benzoate | 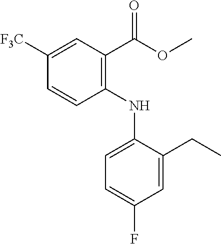 [SMILES: CCc1cc(F)ccc1Nc1ccc(C(F)(F)F)cc1C(=O)OC] | MS (m/z) 342.0 (M + H)⁺ | Methyl 2-bromo- 5-(trifluorometh- yl)benzoate | 2-ethyl-4- fluoroaniline |
| 102 | methyl 5- fluoro-2-((4- fluoro-2- methylphenyl)- amino)-6- methoxy- nicotinate | 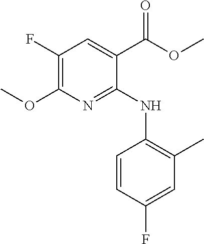 | MS (m/z) 309.0 (M + H)⁺ | methyl 2-chloro- 5-fluoro-6-meth- oxynicotinate | 4-fluoro-2- methylaniline |
| 103 | methyl 5- cyano-2-((4- fluoro-2- methylphen- yl)amino)-4- (trifluorometh- yl)benzoate | 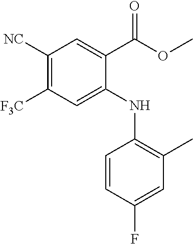 [SMILES: COC(=O)c1cc(C#N)c(C(F)(F)F)cc1Nc1ccc(F)cc1C] | MS (m/z) 353.3 (M + H)⁺ | methyl 2-bromo- 5-cyano-4- (trifluoromethyl)- benzoate | 4-fluoro-2- methyl Aniline |
| 104 | methyl 5- chloro-4- (difluorometh- yl)-2-((4- fluoro-2- methylphen- yl)amino)- benzoate | 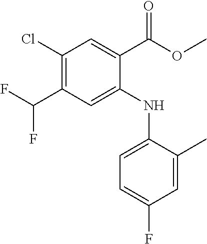 [SMILES: COC(=O)c1cc(Cl)c(C(F)F)cc1Nc1ccc(F)cc1C] | MS (m/z) 344.2 (M + H)⁺ | methyl 2-bromo- 5-chloro-4- (difluoromethyl)- benzoate | 4-fluoro-2- methylaniline |
| 105 | methyl 5- chloro-6- cyano-2-((2- methyl-4- (trifluoro- methoxy)- phenyl)amino)- nicotinate | 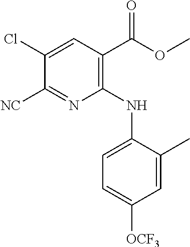 [SMILES: COC(=O)c1cc(Cl)c(C#N)nc1Nc1ccc(OC(F)(F)F)cc1C] | MS (m/z) 386.2 (M + H)⁺ | methyl 2,5- dichloro-6-cyano- nicotinate | 2-methyl-4- (trifluorometh- oxy)aniline |
| 106 | methyl 5- chloro-6- cyano-2-((4- fluoro-2- methylphen- yl)amino)- nicotinate | 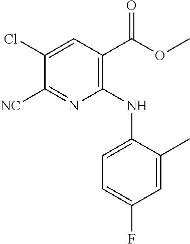 [SMILES: COC(=O)c1cc(Cl)c(C#N)nc1Nc1ccc(F)cc1C] | MS (m/z) 320.2 (M + H)⁺ | methyl 2,5- dichloro-6-cyano- nicotinate | 4-fluoro-2- methylaniline |
| 107 | ethyl 2- ((2,4-dimeth- oxyphenyl)- amino)-5- (trifluorometh- yl)benzoate | 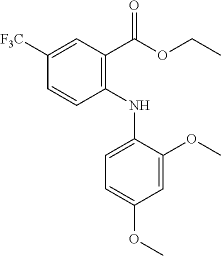 [SMILES: CCOC(=O)c1cc(C(F)(F)F)ccc1Nc1ccc(OC)cc1OC] | MS (m/z) 370.1 (M + H)⁺ | ethyl 2-bromo- 5-(trifluorometh- yl)benzoate | 2,4-dimeth- oxyaniline |
| 108 | ethyl 2-((4- fluoro-2-meth- oxyphenyl)- amino)-5-(tri- fluoromethyl)- benzoate | 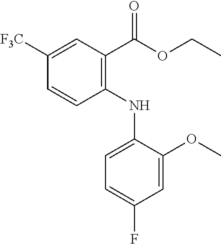 [SMILES: CCOC(=O)c1cc(C(F)(F)F)ccc1Nc1ccc(F)cc1OC] | MS (m/z) 358.1 (M + H)⁺ | ethyl 2-bromo- 5-(trifluorometh- yl)benzoate | 4-fluoro-2- methoxyaniline |
| 109 | methyl 5- chloro-4- fluoro-2- ((2-methyl-4- (trifluoro- methoxy)phen- yl)amino)- benzoate | 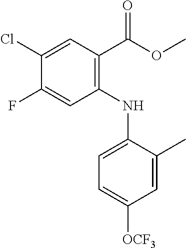 [SMILES: COC(=O)c1cc(Cl)c(F)cc1Nc1ccc(OC(F)(F)F)cc1C] | MS (m/z) 377.1 (M)⁺ | methyl 2-bromo- 5-chloro-4- fluorobenzoate | 2-methyl-4- (trifluorometh- oxy)aniline |
| 110 | methyl 5- chloro-4- fluoro-2-((4- fluoro-2- methylphen- yl)amino)- benzoate | 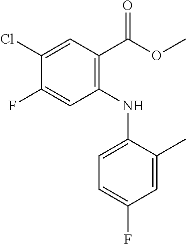 [SMILES: COC(=O)c1cc(Cl)c(F)cc1Nc1ccc(F)cc1C] | MS (m/z) 312.1 (M + H)⁺ | methyl 2-bromo- 5-chloro-4-fluoro- benzoate | 4-fluoro-2- methylaniline |
| 111 | ethyl 2-((4- fluoro-2- isopropylphen- yl)amino)-5- (trifluorometh- yl)benzoate | 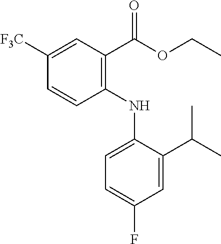 [SMILES: CCOC(=O)c1cc(C(F)(F)F)ccc1Nc1ccc(F)cc1C(C)C] | MS (m/z) 370.1 (M + H)⁺ | ethyl 2-bromo- 5-(trifluorometh- yl)benzoate | 4-fluoro-2-iso- propylaniline |
| 112 | ethyl 6-(di- fluorometh- oxy)-2-((4- fluoro-2- methylphen- yl)amino)- nicoinate | 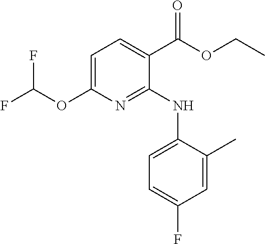 [SMILES: CCOC(=O)c1ccc(OC(F)F)nc1Nc1ccc(F)cc1C] | MS (m/z) 341 (M + H)⁺ | ethyl 2-chloro- 6-(difluorometh- oxy)nicotinate | 4-fluoro-2- methylaniline |
| 113 | methyl 2-((2- (2,2,2-trifluoro- ethyl)phenyl)- amino)-5-(tri- fluorometh- yl)benzoate | 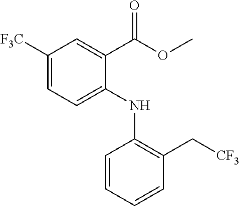 [SMILES: COC(=O)c1cc(C(F)(F)F)ccc1Nc1ccccc1CC(F)(F)F] | MS (m/z) 376 (M + H)⁺ | methyl 2-bromo- 5-(trifluorometh- yl)benzoate | 4-fluoro-2- (2,2,2-tri- fluoroethyl)- aniline |
| 114 | 2-((2-cyclo- propyl-4- fluorophenyl)- amino)-4-(tri- fluoromethyl)- benzoate | 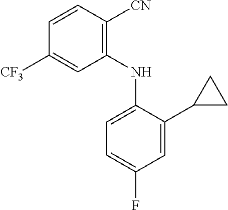 [SMILES: N#Cc1ccc(C(F)(F)F)cc1Nc1ccc(F)cc1C1CC1] | MS (m/z) 321.1 (M + H)⁺ | 2-bromo-4-(tri- fluoromethyl)- benzonitrile | 2-cyclopropyl- 4-fluoroaniline |
| 115 | 4-(difluoro- methoxy)-2- ((4-fluoro-2- methylphen- yl)amino)- benzonitrile | 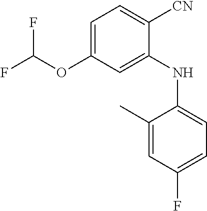 [SMILES: Cc1cc(F)ccc1Nc1cc(OC(F)F)ccc1C#N] | MS (m/z) 291.3 (M − H)⁻ | 2-bromo-4- (difluorometh- oxy)benzonitrile | 4-fluoro-2- methylaniline |
| 116 | 4-cyclopropyl- 2-((4-fluoro-2- methylphen- yl)amino)- benzonitrile | 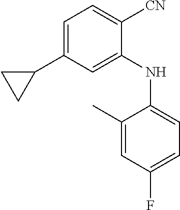 [SMILES: Cc1cc(F)ccc1Nc1cc(C2CC2)ccc1C#N] | MS (m/z) 267.2 (M − H)⁻ | 2-bromo-4-cyclo- propylbenzonitrile | 4-fluoro-2- methylaniline |
| 117 | methyl 5- fluoro-2-((4- fluoro-2- isopropylphen- yl)amino)- benzoate | 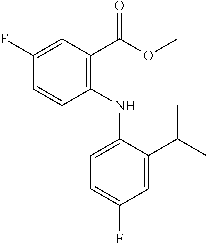 [SMILES: COC(=O)c1cc(F)ccc1Nc1ccc(F)cc1C(C)C] | MS (m/z) 306.2 (M + H)⁺ | methyl 2-bromo- 5-fluorobenzoate | 4-fluoro-2- isopropyl- aniline |
| 118 | methyl 2-((4- (difluorometh- oxy)-2-meth- yl)phenyl)- amino)-5- (trifluorometh- yl)benzoate | 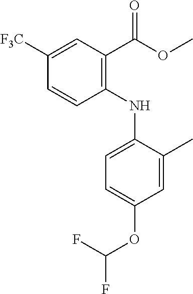 [SMILES: COC(=O)c1cc(C(F)(F)F)ccc1Nc1ccc(OC(F)F)cc1C] | MS (m/z) 376.3 (M + H)⁺ | methyl 2-bromo- 5-(trifluorometh- yl)benzoate | 4-(difluoro- methoxy)-2- methylaniline |
| 119 | methyl 5- chloro-2- ((2-ethyl-4- fluorophenyl)- amino)-4- fluorobenzoate | 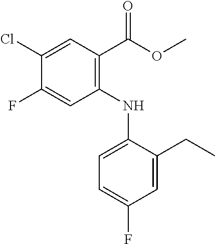 [SMILES: CCc1cc(F)ccc1Nc1cc(F)c(Cl)cc1C(=O)OC] | MS (m/z) 326.2 (M + H)⁺ | methyl 2-bromo- 5-chloro-4-fluoro- benzoate | 2-ethyl-4- fluoroaniline |
| 120 | methyl 2-((4- fluoro-2- methylphenyl)- amino)-6-meth- ylnicotinate | 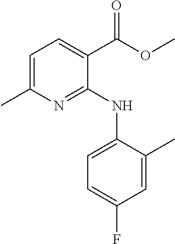 | MS (m/z) 275.3 (M + H)⁺ | methyl 2-bromo- 6-methylnicotinate | 4-fluoro-2- methylaniline |
| 121 | methyl 5- chloro-6- cyano-2-((2- ethyl-4-fluoro- phenyl)amino)- nicotinate | 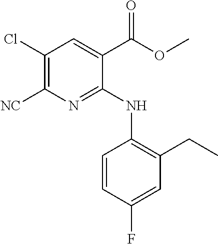 [SMILES: CCc1cc(F)ccc1Nc1nc(C#N)c(Cl)cc1C(=O)OC] | MS (m/z) 334.2 (M + H)⁺ | methyl 2,5- dichloro-6-cyano- nicotinate | 2-ethyl-4- fluoroaniline |
| 122 | 2-((4-fluoro-2- isopropylphen- yl)amino)-6- (trifluorometh- yl)nicotino- nitrile | 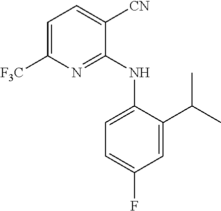 [SMILES: CC(C)c1cc(F)ccc1Nc1nc(C(F)(F)F)ccc1C#N] | MS (m/z) 324.2 (M + H)⁺ | 2-chloro-6-(tri- fluoromethyl)- nicotinonitrile | 4-fluoro-2-iso- propylaniline |
| 123 | 5-chloro-2-((4- fluoro-2- isopropylphen- yl)amino)- nicotinonitrile | 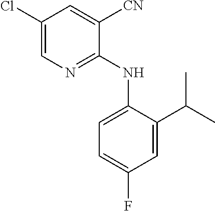 [SMILES: CC(C)c1cc(F)ccc1Nc1ncc(Cl)cc1C#N] | MS (m/z) 290.1 (M + H)⁺ | 2,5-dichloro- nicotinonitrile | 4-fluoro-2-iso- propylaniline |
| 124 | methyl 2-((4- fluoro-2- isopropylphen- yl)amino)-5- (trifluorometh- yl)nicotinate | 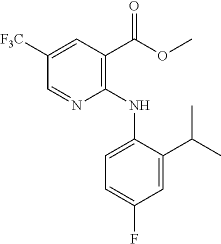 [SMILES: COC(=O)c1cc(C(F)(F)F)cnc1Nc1ccc(F)cc1C(C)C] | MS (m/z) 357.2 (M + H)⁺ | methyl 2-chloro- 5-(trifluorometh- yl)nicotinate | 4-fluoro-2-iso- propylaniline |
| 125 | methyl 4-(di- methylamino)- 2-((4-fluoro-2- methylphenyl)- amino)benzoate | 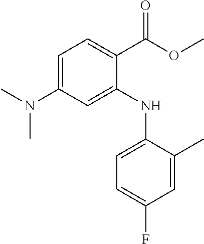 | MS (m/z) 303.0 (M + H)⁺ | methyl-2-bromo- 4-(dimethyl- amino)benzoate | 4-fluoro-2- methylaniline |
| 126 | ethyl 2-((4- fluoro-2-iso- propylphenyl)- amino)-4-meth- ylbenzoate | 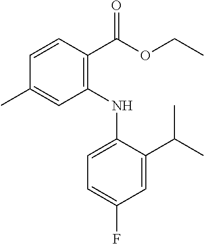 [SMILES: CCOC(=O)c1ccc(C)cc1Nc1ccc(F)cc1C(C)C] | MS (m/z) 316.1 (M + H)⁺ | ethyl 2-bromo- 4-methylbenzoate | 4-fluoro-2- isopropyl- aniline |
| 127 | methyl 4-(di- fluoromethyl)- 5-fluoro-2- ((4-fluoro-2- methylphenyl)- amino)benzoate | 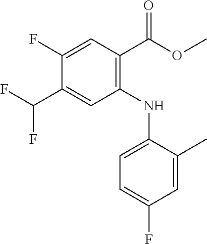 [SMILES: COC(=O)c1cc(F)c(C(F)F)cc1Nc1ccc(F)cc1C] | MS (m/z) 328.0 (M + H)⁺ | methyl 2-bromo- 4-(difluorometh- yl)-5-fluoro- benzoate | 4-fluoro-2- methylaniline |
| 128 | methyl 5- fluoro-2-((4- fluoro-2-meth- ylphenyl)-amino)-6-meth- ylnicotinate | 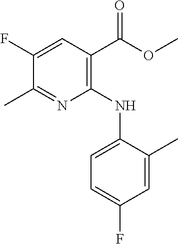 [SMILES: COC(=O)c1cc(F)c(C)nc1Nc1ccc(F)cc1C] | MS (m/z) 293.0 (M + H)⁺ | methyl 2-chloro- 5-fluoro-6-meth- ylnicotinate | 4-fluoro-2- methylaniline |
| 129 | methyl 5- chloro-2-((4- fluoro-2-meth- ylphenyl)- amino)-6-meth- ylnicotinate | 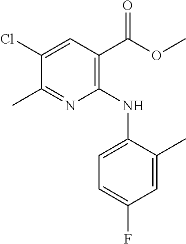 [SMILES: COC(=O)c1cc(Cl)c(C)nc1Nc1ccc(F)cc1C] | MS (m/z) 309.2 (M + H)⁺ | methyl 5-chloro- 2-((4-fluoro-2- methylphenyl)- amino)-6-methyl- nicotinate | 4-fluoro-2- methylaniline |
| 130 | methyl 5,6- dichloro-2- ((4-fluoro-2- methylphen- yl)amino)- nicotinate | 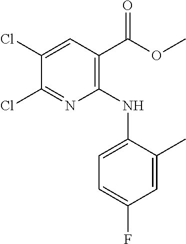 [SMILES: COC(=O)c1cc(Cl)c(Cl)nc1Nc1ccc(F)cc1C] | MS (m/z) 329.0 (M + H)⁺ | methyl 2,5,6-tri- chloronicotinate | 4-fluoro-2- methylaniline |
| 131 | methyl 3- chloro-2- fluoro-6-((2- methyl-4- (trifluorometh- oxy)phenyl)- amino)benzoate | 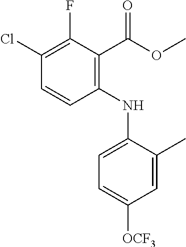 [SMILES: COC(=O)c1c(Nc2ccc(OC(F)(F)F)cc2C)ccc(Cl)c1F] | MS (m/z) 378.0 (M + H)⁺ | methyl 6-bromo- 3-chloro-2- fluorobenzoate | 2-methyl-4- (trifluorometh- oxy)aniline |
| 132 | ethyl 5-cyano- 2-((4-fluoro- 2-methylphen- yl)amino)- benzoate | 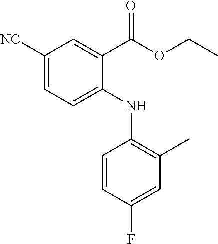 [SMILES: CCOC(=O)c1cc(C#N)ccc1Nc1ccc(F)cc1C] | MS (m/z) 297.0 (M − H)⁻ | ethyl 2-bromo-5- cyanobenzoate | 4-fluoro-2- methylaniline |
| 133 | ethyl 2-((4- fluoro-3-meth- oxy-2-meth- ylphenyl)- amino)-4-(tri- fluoromethyl)- benzoate | 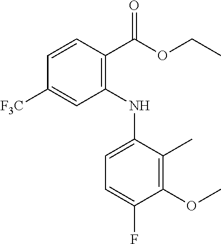 [SMILES: CCOC(=O)c1ccc(C(F)(F)F)cc1Nc1ccc(F)c(OC)c1C] | MS (m/z) 372.2 (M + H)⁺ | ethyl 2-bromo- 4-(trifluorometh- yl)benzoate | 4-fluoro-3- methoxy-2- methoxy-2- methylaniline |
| 134 | methyl 3- chloro-2- fluoro-6-((4- fluoro-2-meth- ylphenyl)- amino)benzoate | 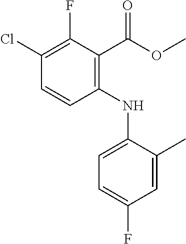 [SMILES: COC(=O)c1c(Nc2ccc(F)cc2C)ccc(Cl)c1F] | MS (m/z) 312.0 (M + H)⁺ | methyl 6-bromo- 3-chloro-2-fluoro- benzoate | 4-fluoro-2- methylaniline |
| 135 | ethyl 4-cyano- 2-((4-fluoro-2- methoxyphen- yl)amino)- benzoate | 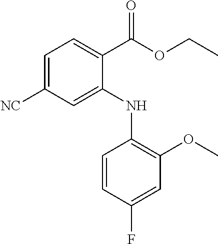 [SMILES: CCOC(=O)c1ccc(C#N)cc1Nc1ccc(F)cc1OC] | MS (m/z) 315.0 (M)⁺ | ethyl 2-bromo-4- cyanobenzoate | 4-fluoro-2- methoxy- aniline |
| 136 | ethyl 2-((4- fluoro-2-meth- ylphenyl)- amino)-4- (methylsulfon- yl)benzoate | 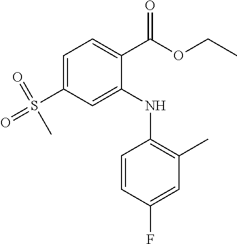 [SMILES: CCOC(=O)c1ccc(S(C)(=O)=O)cc1Nc1ccc(F)cc1C] | MS (m/z) 352.0 (M + H)⁺ | ethyl 2-bromo-4- (methylsulfonyl)- benzoate | 4-fluoro-2- methylaniline |
| 137 | methyl 5- chloro-2-((2- ethyl-4- fluorophenyl)- amino)benzoate | 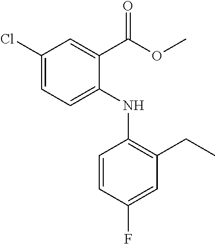 [SMILES: CCc1cc(F)ccc1Nc1ccc(Cl)cc1C(=O)OC] | MS (m/z) 308.0 (M + H)⁺ | methyl 2-bromo- 5-chlorobenzoate | (2-ethyl-4- fluoroaniline |
| 138 | ethyl 2-((4- fluoro-2- methylphenyl)- amino)-4-(2,2,2-tri- fluoroethoxy)- benzoate | 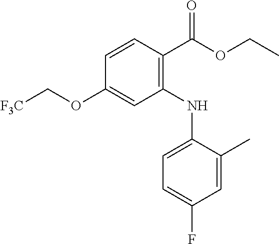 [SMILES: CCOC(=O)c1ccc(OCC(F)(F)F)cc1Nc1ccc(F)cc1C] | MS (m/z) 372.0 (M + H)⁺ | ethyl 2-bromo-4- (2,2,2-trifluoro- ethoxy)benzoate | 4-fluoro-2- methylaniline |
| 139 | methyl 5- fluoro-2-((4- fluoro-2- methylphenyl)- amino)-4-meth- ylbenzoate | 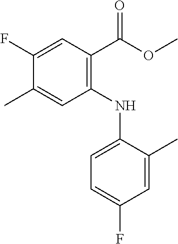 | MS (m/z) 292.0 (M + H)⁺ | methyl 2-bromo- 5-fluoro-4-meth- ylbenzoate | 4-fluoro-2- methylaniline |
| 140 | ethyl 2-chloro- 6-((4-fluoro-2- methylphenyl)- amino)benzoate | 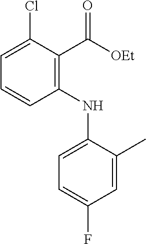 [SMILES: CCOC(=O)c1c(Cl)cccc1Nc1ccc(F)cc1C] | MS (m/z) 308.0 (M + H)⁺ | ethyl 2-bromo- 6-chlorobenzoate | 4-fluoro-2- methylaniline |
| 141 | methyl 5- chloro-2-((4- fluoro-2-meth- oxyphenyl)- amino)benzoate | 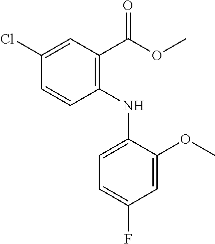 [SMILES: COC(=O)c1cc(Cl)ccc1Nc1ccc(F)cc1OC] | MS (m/z) 310.0 (M + H)⁺ | methyl 2-bromo- 5-chlorobenzoate | 4-fluoro-2- methoxyaniline |
| 142 | methyl 4- fluoro-2-((4- fluoro-2-iso- propylphenyl)- amino)benzoate | 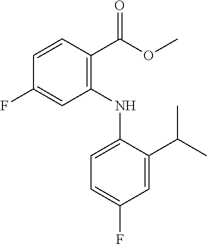 [SMILES: COC(=O)c1ccc(F)cc1Nc1ccc(F)cc1C(C)C] | MS (m/z) 306.2 (M + H)⁺ | methyl 2-bromo- 4-fluorobenzoate | 4-fluoro-2- isopropylaniline |
| 143 | methyl 2-((2- (tert-butyl)-4- fluorophenyl)- amino)-5- chlorobenzoate | 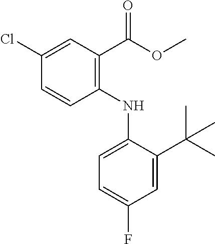 [SMILES: COC(=O)c1cc(Cl)ccc1Nc1ccc(F)cc1C(C)(C)C] | MS (m/z) 336.0 (M + H)⁺ | methyl 2-bromo- 5-chlorobenzoate | 2-(tertbutyl)- 4-fluoroaniline |
| 144 | methyl 3- chloro-6-((4- fluoro-2-iso- propylphenyl)- amino)-2- methylbenzoate | 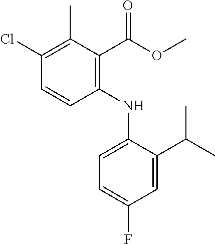 [SMILES: COC(=O)c1c(Nc2ccc(F)cc2C(C)C)ccc(Cl)c1C] | MS (m/z) 336.0 (M + H)⁺ | methyl 6-bromo- 3-chloro-2-meth- ylbenzoate | 4-fluoro-2-iso- propylaniline |
| 145 | methyl 2-((2- methyl-4- (trifluorometh- oxy)phenyl)- amino)-5-(tri- fluoromethyl)- benzoate | 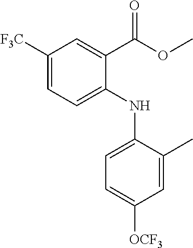 [SMILES: COC(=O)c1cc(C(F)(F)F)ccc1Nc1ccc(OC(F)(F)F)cc1C] | MS (m/z) 393.8 (M)⁺ | methyl 2-fromo-5- (trifluoromethyl)- benzoate | 2-methyl-4- (trifluorometh- oxy)aniline |
| 146 | methyl 5- chloro-2- ((4-fluoro-2- methylphenyl)- amino)- nicotinate | 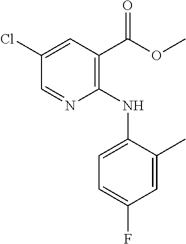 [SMILES: COC(=O)c1cc(Cl)cnc1Nc1ccc(F)cc1C] | MS (m/z) 295.0 (M + H)⁺ | methyl 2,5-di- chloronicotinate | 4-fluoro-2- methylaniline |
| 147 | methyl 5- chloro-4- fluoro-2-((4- fluoro-2-iso- propylphenyl)- amino)benzoate | 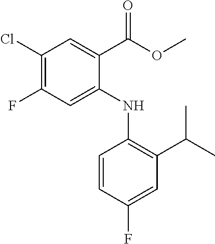 [SMILES: COC(=O)c1cc(Cl)c(F)cc1Nc1ccc(F)cc1C(C)C] | MS (m/z) 340.0 (M + H)⁺ | methyl 2-bromo- 5-chloro-4-fluoro- benzoate | 4-fluoro-2-iso- propylaniline |
| 148 | ethyl 2-((4- fluoro-2- methylphenyl)- amino)-5- methoxy-4-(tri- fluoromethyl)- benzoate | 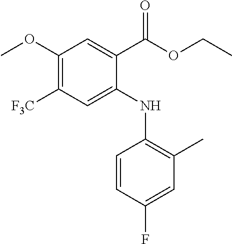 [SMILES: CCOC(=O)c1cc(OC)c(C(F)(F)F)cc1Nc1ccc(F)cc1C] | MS (m/z) 372.0 (M + H)⁺ | ethyl 2-bromo-5- methoxy-4- (trifluoromethyl)- benzoate | 4-fluoro-2- methylaniline |
| 149 | ethyl 2-((4- fluoro-2-meth- ylphenyl)- amino)-4-meth- oxybenzoate | 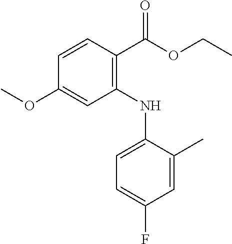 [SMILES: CCOC(=O)c1ccc(OC)cc1Nc1ccc(F)cc1C] | MS (m/z) 304.0 (M + H)⁺ | ethyl 2-bromo-4- methoxybenzoate | 4-fluoro-2- methylaniline |
| 150 | methyl 2-((4- fluoro-2-iso- propylphenyl)- amino)-4- (trifluorometh- oxy)benzoate | 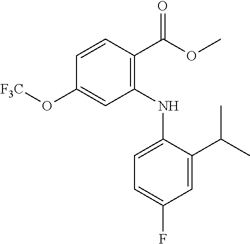 [SMILES: COC(=O)c1ccc(OC(F)(F)F)cc1Nc1ccc(F)cc1C(C)C] | MS (m/z) 371.8 (M + H)⁺ | methyl 2-bromo- 4-(trifluorometh- oxy)benzoate | 4-fluoro-2-iso- propylaniline |
| 151 | methyl 5- chloro-4- fluoro-2-((4- fluoro-2- methylphenyl)- amino)benzoate | 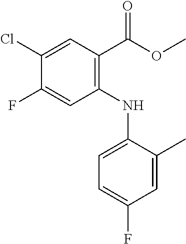 [SMILES: COC(=O)c1cc(Cl)c(F)cc1Nc1ccc(F)cc1C] | MS (m/z) 312.0 (M + H)⁺ | methyl 2-bromo- 5-chloro-4- fluorobenzoate | 4-fluoro-2- methylaniline |
| 152 | methyl 2- fluoro-6-((4- fluoro-2-meth- ylphenyl)- amino)-3-(tri- fluoromethyl)- benzoate | 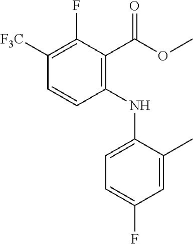 [SMILES: COC(=O)c1c(Nc2ccc(F)cc2C)ccc(C(F)(F)F)c1F] | MS (m/z) 344.0 (M + H)⁺ | methyl 6-bromo- 2-fluoro-3-(tri- fluoromethyl)- benzoate | 4-fluoro-2- methylaniline |
| 153 | methyl 3- chloro-6-((4- fluoro-2-meth- ylphenyl)- amino)-2-meth- ylbenzoate | 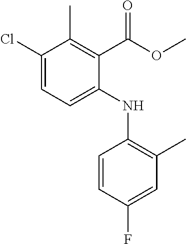 [SMILES: COC(=O)c1c(Nc2ccc(F)cc2C)ccc(Cl)c1C] | MS (m/z) 308.0 (M + H)⁺ | methyl 6-bromo- 3-chloro-2-meth- ylbenzoate | 4-fluoro-2- methylaniline |
| 154 | ethyl 2-((2- (tert-butyl)-4- fluorophenyl)- amino)-4-(tri- fluoromethyl)- benzoate | 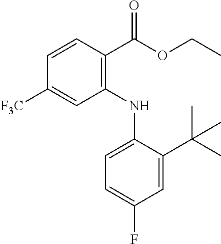 [SMILES: CCOC(=O)c1ccc(C(F)(F)F)cc1Nc1ccc(F)cc1C(C)(C)C] | MS (m/z) 384.1 (M + H)⁺ | ethyl 2-bromo- 4-(trifluorometh- yl)benzoate | 2-(tertbutyl)- 4-fluoroaniline |
| 155 | ethyl 2-((4- fluoro-2-iso- propylphenyl)- amino)-6-meth- oxybenzoate | 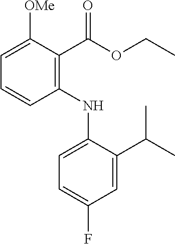 [SMILES: CCOC(=O)c1c(Nc2ccc(F)cc2C(C)C)cccc1OC] | MS (m/z) 332.2 (M + H)⁺ | ethyl 2-bromo-6- methoxybenzoate | 4-fluoro-2-iso- propylaniline |
| 156 | methyl 2-((2- ethyl-4-fluoro- phenyl)amino)- 5-fluoro-benzoate | 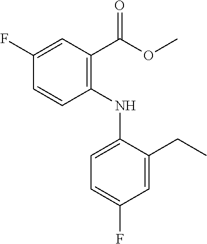 [SMILES: CCc1cc(F)ccc1Nc1ccc(F)cc1C(=O)OC] | MS (m/z) 292.2 (M + H)⁺ | methyl 2-bromo- 5-fluorobenzoate | 2-ethyl-4- fluoroaniline |
| 157 | methyl 2-((2- methyl-4- (trifluorometh- oxy)phenyl)- amino)-5-(tri- fluoromethyl)- nicotinate | 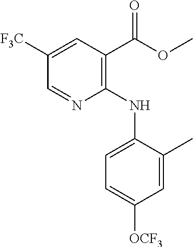 [SMILES: COC(=O)c1cc(C(F)(F)F)cnc1Nc1ccc(OC(F)(F)F)cc1C] | MS (m/z) 394.8 (M + H)⁺ | methyl 2-chloro- 5-(trifluorometh- yl)nicotinate | 2-methyl-4- (trifluorometh- oxy)aniline |
| 158 | methyl 6-(di- fluoromethyl)- 2-((4-fluoro- 2-isopropyl- phenyl)amino)- nicotinate | 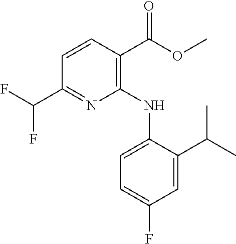 [SMILES: COC(=O)c1ccc(C(F)F)nc1Nc1ccc(F)cc1C(C)C] | MS (m/z) 339.0 (M + H)⁺ | methyl 2-chloro- 6-(difluorometh- yl)nicotinate | 4-fluoro-2-iso- propylaniline |
| 159 | methyl 2-((4- cyano-2-meth- ylphenyl)- amino)-5- (trifluorometh- oxy)benzoate | 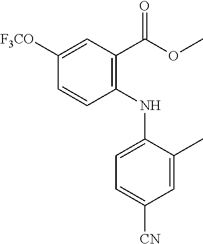 [SMILES: COC(=O)c1cc(OC(F)(F)F)ccc1Nc1ccc(C#N)cc1C] | MS (m/z) 351.0 (M + H)⁺ | methyl 2-bromo- 5-(trifluorometh- oxy)benzoate | 4-amino-3- methylbenzo- nitrile |
| 160 | ethyl 3-chloro- 2-((4-fluoro-2- methylphenyl)- amino)benzoate | 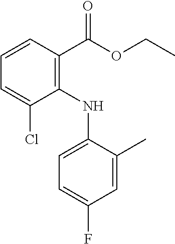 [SMILES: CCOC(=O)c1cccc(Cl)c1Nc1ccc(F)cc1C] | MS (m/z) 308.0 (M + H)⁺ | ethyl 2-bromo-3- chlorobenzoate | 4-fluoro-2- methylaniline |
| 161 | ethyl 2-(di- fluoromethyl)- 6-((4-fluoro- 2-methylphen- yl)amino)- benzoate | 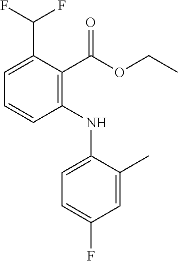 [SMILES: CCOC(=O)c1c(Nc2ccc(F)cc2C)cccc1C(F)F] | MS (m/z) 324.0 (M + H)⁺ | ethyl 2-bromo-6- (difluoromethyl)- benzoate | 4-fluoro-2- methylaniline |
| 162 | methyl 3- chloro-2- fluoro-6-((4- fluoro-2-meth- ylphenyl)- amino)benzoate | 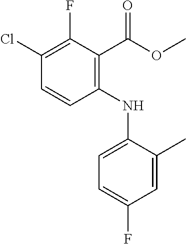 [SMILES: COC(=O)c1c(Nc2ccc(F)cc2C)ccc(Cl)c1F] | MS (m/z) 312.0 (M + H)⁺ | methyl 6-bromo- 3-chloro-2- fluorobenzoate | 4-fluoro-2- methylaniline |
| 163 | methyl 4,5- difluoro-2- ((4-fluoro-2- methoxyphen- yl)amino)- benzoate | 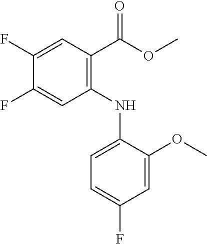 [SMILES: COC(=O)c1cc(F)c(F)cc1Nc1ccc(F)cc1OC] | MS (m/z) 312.0 (M + H)⁺ | methyl 2-bromo- 4,5-difluoro- benzoate | 4-fluoro-2- methoxyaniline |
| 164 | methyl 5- chloro-2-((2- methoxy-4- methylphenyl)- amino)benzoate | 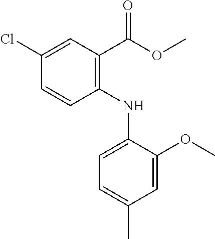 [SMILES: COC(=O)c1cc(Cl)ccc1Nc1ccc(C)cc1OC] | MS (m/z) 306.0 (M + H)⁺ | methyl 2-bromo- 5-chlorobenzoate | 2-methoxy-4- methylaniline |
| 165 | ethyl 2-((2- fluoro-6-meth- ylphenyl)- amino)-5- (trifluorometh- yl)benzoate | 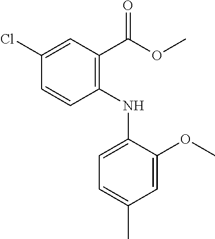 | MS (m/z) 342.0 (M + H)⁺ | ethyl 2-bromo-5- (trifluoromethyl)- benzoate | 2-fluoro-6- methylaniline |
| 166 | methyl 4-(di- fluoromethyl)- 2-((4-fluoro- 2-isopropyl- phenyl)amino)- benzoate | 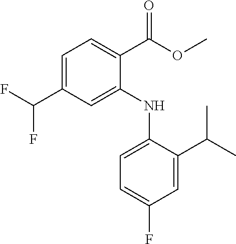 [SMILES: COC(=O)c1ccc(C(F)F)cc1Nc1ccc(F)cc1C(C)C] | MS (m/z) 338.1 (M + H)⁺ | methyl 2-bromo- 4-(difluorometh- yl)benzoate | 4-fluoro-2-iso- propylaniline |
| 167 | methyl 4-(di- fluoromethyl)- 2-((4-fluoro- 2-methylphen- yl)amino)- benzoate | 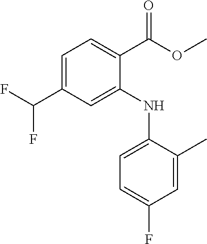 [SMILES: COC(=O)c1ccc(C(F)F)cc1Nc1ccc(F)cc1C] | MS (m/z) 310.0 (M + H)⁺ | methyl 2-bromo- 4-(difluorometh- yl)benzoate | 4-fluoro-2- methylaniline |
| 168 | methyl 5- chloro-2-((4- fluoro-2-iso- propylphen- yl)amino)- benzoate | 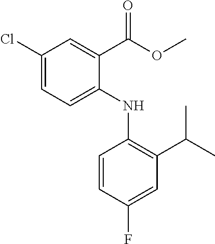 [SMILES: COC(=O)c1cc(Cl)ccc1Nc1ccc(F)cc1C(C)C] | MS (m/z) 322.0 (M + H)⁺ | methyl 2-bromo- 5-chlorobenzoate | 4-fluoro-2-iso- propylaniline |
| 169 | methyl 5- fluoro-2-((4- fluoro-2-iso- propylphenyl)- amino)nico- tinate | 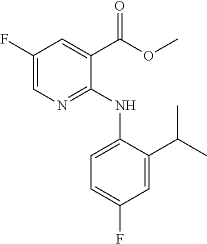 [SMILES: COC(=O)c1cc(F)cnc1Nc1ccc(F)cc1C(C)C] | MS (m/z) 307.0 (M + H)⁺ | methyl 2-bromo- 5-fluoronicotinate | 4-fluoro-2-iso- propylaniline |
| 170 | methyl 5- fluoro-2-((4- fluoro-2- methylphenyl)- amino)benzoate | 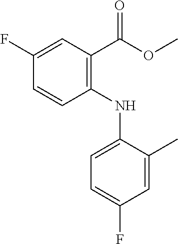 [SMILES: COC(=O)c1cc(F)ccc1Nc1ccc(F)cc1C] | MS (m/z) 278.0 (M + H)⁺ | methyl 2-bromo- 5-fluorobenzoate | 4-fluoro-2- methylaniline |
| 171 | N-(2-((4- fluoro-2-meth- ylphenyl)- amino)-5- (trifluorometh- yl)benzyl)-6- methoxy-2- methylpyridin- 3-amine | 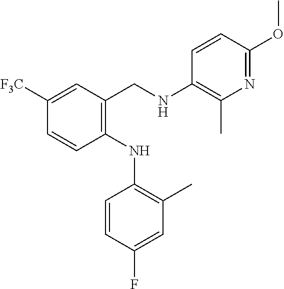 [SMILES: COc1ccc(NCc2cc(C(F)(F)F)ccc2Nc2ccc(F)cc2C)c(C)n1] | MS (m/z) 420.0 (M + H)⁺ | N-(2-bromo-5- (trifluoromethyl)- benzyl)-6-meth- oxy-2-methyl- pyridin-3-amine | 4-fluoro-2- methylaniline |

### Intermediate 172

### methyl 2-((4-methylthiazol-5-yl)amino)-5-(trifluoromethyl)benzoate

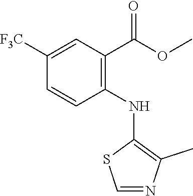 [SMILES: COC(=O)c1cc(C(F)(F)F)ccc1Nc1scnc1C]

A 250 mL sealed tube, fitted with a magnetic stir bar, was charged with methyl 2-amino-5-(trifluoromethyl)benzoate (1 g, 4.56 mmol), 5-bromo-4-methylthiazole (0.894 g, 5.02 mmol) and cesium carbonate (2.97 g, 9.13 mmol). 1,4-Dioxane (20 mL) was added and the resulting reaction mixture was purged with nitrogen for 10 minutes before xantphos (0.264 g, 0.456 mmol) and Pd₂(dba)₃ (0.209 g, 0.228 mmol) were added. The reaction mixture was stirred at 100° C. for 16 hours and then cooled to RT. The reaction was diluted with ethyl acetate (20 mL) and filtered through a Celite pad. The Celite pad was washed with ethyl acetate (80 mL) and the filtrate was concentrated under reduced pressure. The crude product was purified by column chromatography (Biotage, 50 g SNAP column, 0-40% EtOAc/petroleum ether over 30 min) to give the title compound as an orange gum (440 mg, 1.165 mmol, 25.5% yield). MS (m/z) 317.0 (M+H)⁺

Intermediates 173-194 were prepared from the indicated aryl halogen and aniline by methods analogous to those described for Intermediate 172.

| Int. | Name | Structure | Characterization | Aryl halogen | Aniline |
|---|---|---|---|---|---|
| 173 | methyl 3-chloro- 4-cyano-2- fluoro-6-((4- fluoro-2-meth- ylphenyl)- amino)benzoate | 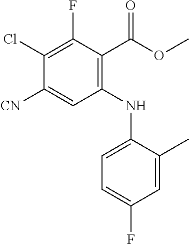 | MS (m/z) 337.2 (M + H)⁺ | 4-fluoro-1-iodo- 2-methylbenzene | methyl 6-amino- 3-chloro-4-cyano- 2-fluorobenzoate |
| 174 | methyl 3-chloro- 2,4-difluoro-6- ((4-fluoro-2-meth- ylphenyl)amino)- benzoate | 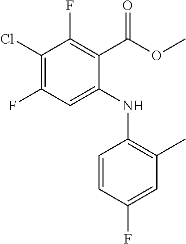 [SMILES: COC(=O)c1c(Nc2ccc(F)cc2C)cc(F)c(Cl)c1F] | MS (m/z) 330.2 (M + H)⁺ | 4-fluoro-1-iodo- 2-methylbenzene | methyl 6-amino- 3-chloro-2,4- difluorobenzoate |
| 175 | methyl 2-((2- methyl-4-(tri- fluoromethyl)- phenyl)amino)- 5-(trifluorometh- yl)nicotinate | 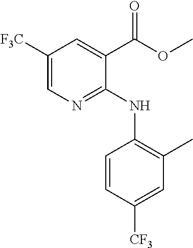 [SMILES: COC(=O)c1cc(C(F)(F)F)cnc1Nc1ccc(C(F)(F)F)cc1C] | MS (m/z) 379.3 (M + H)⁺ | 1-bromo-2-meth- yl-4-(trifluoro- methyl)benzene | methyl 2-amino- 5-(trifluorometh- yl)nicotinate |
| 176 | methyl 2-((4- chloro-2-methyl- phenyl)amino)- 5-(trifluorometh- yl)nicotinate | 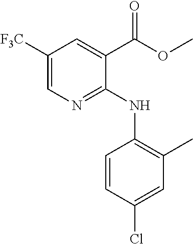 [SMILES: COC(=O)c1cc(C(F)(F)F)cnc1Nc1ccc(Cl)cc1C] | MS (m/z) 345.2 (M + H)⁺ | 4-chloro-1-iodo- 2-methylbenzene | methyl 2-amino- 5-(trifluorometh- yl)nicotinate |
| 177 | 4-chloro-5-fluoro- 2-((4-fluoro-2- methylphenyl)- amino)benzoic acid | 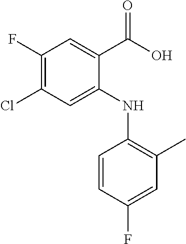 [SMILES: Cc1cc(F)ccc1Nc1cc(Cl)c(F)cc1C(=O)O] | MS (m/z) 298.1 (M + H)⁺ | 1-bromo-4-fluoro- 2-methylbenzene | 2-amino-4-chloro- 5-fluorobenzoic acid |
| 178 | ethyl 5-chloro-2- ((2-ethoxy-4- fluorophenyl)- amino)benzoate | 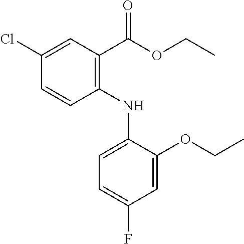 [SMILES: CCOC(=O)c1cc(Cl)ccc1Nc1ccc(F)cc1OCC] | MS (m/z) 338.0 (M + H)⁺ | 1-bromo-2-ethoxy- 4-fluorobenzene | 1 ethyl 2-amino- 5-chlorobenzoate |
| 179 | methyl 2-((3- methylthiophen- 2-yl)amino)-5- (trifluoromethyl)- benzoate | 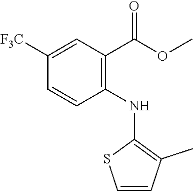 [SMILES: COC(=O)c1cc(C(F)(F)F)ccc1Nc1sccc1C] | MS (m/z) 315.8 (M + H)⁺ | 2-bromo-3-meth- ylthiophene | methyl 2-amino- 5-(trifluorometh- yl)benzoate |
| 180 | methyl 2-((3,4- difluoro-2-meth- ylphenyl)amino)- 5-(trifluorometh- yl)benzoate | 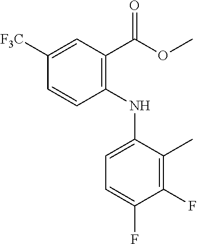 [SMILES: COC(=O)c1cc(C(F)(F)F)ccc1Nc1ccc(F)c(F)c1C] | MS (m/z) 346.0 (M + H)⁺ | 1-bromo-3,4- difluoro-2-meth- ylbenzene | methyl 2-amino- 5-(trifluorometh- yl)benzoate |
| 181 | methyl 4-chloro- 5-cyano-2-((4- fluoro-2-methyl- phenyl)amino)- benzoate | 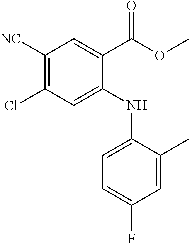 [SMILES: COC(=O)c1cc(C#N)c(Cl)cc1Nc1ccc(F)cc1C] | MS (m/z) 317.0 (M + H)⁺ | 4-fluoro-1-iodo- 2-methylbenzene | methyl 2-amino- 4-chloro-5-cyano- benzoate |
| 182 | methyl 4-fluoro- 2-((4-fluoro-2- methylphenyl)- amino)-5-(tri- fluoromethyl)- benzoate | 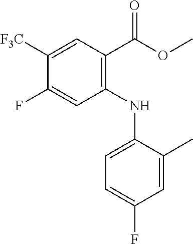 [SMILES: COC(=O)c1cc(C(F)(F)F)c(F)cc1Nc1ccc(F)cc1C] | MS (m/z) 344.2 (M − H)⁻ | 4-fluoro-1-iodo- 2-methylbenzene | methyl 2-amino- 2-fluoro-5-(tri- fluoromethyl)- benzoate |
| 183 | methyl 5-fluoro- 2-((4-fluoro-2- methylphenyl)- amino)-4-(tri- fluoromethyl)- benzoate | 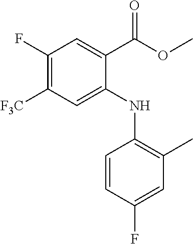 [SMILES: COC(=O)c1cc(F)c(C(F)(F)F)cc1Nc1ccc(F)cc1C] | GCMS (m/z) 345 (M)⁺ | 4-fluoro-1-iodo- 2-methylbenzene | methyl 2-amino- 5-fluoro-4-(tri- fluoromethyl)- benzoate |
| 184 | methyl 5-bromo- 4-fluoro-2-((4- fluoro-2-methyl- phenyl)amino)- benzoate | 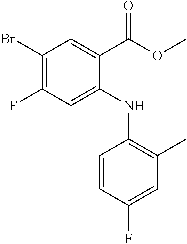 [SMILES: COC(=O)c1cc(Br)c(F)cc1Nc1ccc(F)cc1C] | MS (m/z) 354.9(M)⁺/355.9 (M + H)⁺ | 4-fluoro-1-iodo- 2-methylbenzene | methyl 2-amino- 5-bromo-4-fluoro- benzoate |
| 185 | ethyl 5-bromo- 2-((4-fluoro-2- methylphenyl)- amino)-4-(tri- fluoromethoxy)- benzoate | 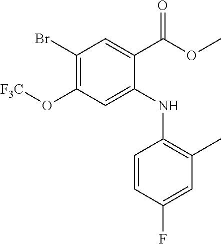 | MS (m/z) 435.8 (M)⁺ | 4-fluoro-1-iodo- 2-methylbenzene | ethyl-2-amino- 5-bromo-4-(tri- fluoromethoxy)- benzoate |
| 186 | methyl 2-((4,5- difluoro-2-meth- ylphenyl)amino)- 5-(trifluorometh- yl)benzoate | 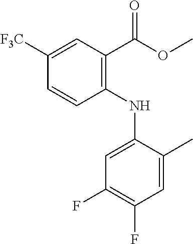 [SMILES: COC(=O)c1cc(C(F)(F)F)ccc1Nc1cc(F)c(F)cc1C] | MS (m/z) 346.0 (M + H)⁺ | 1-bromo-4,5- difluoro-2-meth- ylbenzene | methyl 2-amino- 5-(trifluorometh- yl)benzoate |
| 187 | methyl 2-((3,5- difluoro-2-meth- ylphenyl)amino)- 5-(trifluorometh- yl)benzoate | 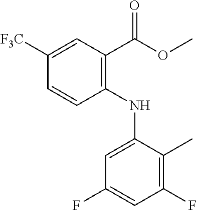 [SMILES: COC(=O)c1cc(C(F)(F)F)ccc1Nc1cc(F)cc(F)c1C] | MS (m/z) 346.0 (M + H)⁺ | 1,5-difluoro-3- iodo-2-methyl- benzene | methyl-2-amino- 5-(trifluorometh- yl)benzoate |
| 188 | ethyl 5-chloro-2- ((4-cyano-2-meth- ylphenyl)amino)- benzoate | 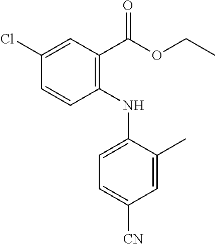 [SMILES: CCOC(=O)c1cc(Cl)ccc1Nc1ccc(C#N)cc1C] | MS (m/z) 315.0 (M + H)⁺ | 4-bromo-3-meth- ylbenzonitrile | ethyl-2-amino- 5-chlorobenzoate |
| 189 | methyl 2-((2,4- difluoro-6-meth- ylphenyl)amino)- 5-(trifluorometh- yl)benzoate | 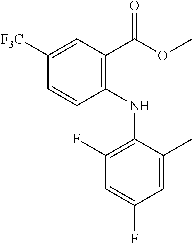 [SMILES: COC(=O)c1cc(C(F)(F)F)ccc1Nc1c(C)cc(F)cc1F] | MS (m/z) 346.0 (M + H)⁺ | 2-bromo-1,5- difluoro-3-meth- ylbenzene | methyl 2-amino- 5-(trifluorometh- yl)benzoate |
| 190 | methyl 2-((4- methoxy-2-meth- ylphenyl)amino)- 5-(trifluorometh- yl)benzoate | 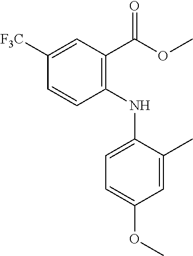 [SMILES: COC(=O)c1cc(C(F)(F)F)ccc1Nc1ccc(OC)cc1C] | MS (m/z) 340.0 (M + H)⁺ | 1-bromo-4-meth- oxy-2-methyl- benzene | methyl 2-amino- 5-(trifluorometh- yl)benzoate |
| 191 | ethyl 5-chloro-2- ((3-cyano-2-meth- ylphenyl)amino)- benzoate | 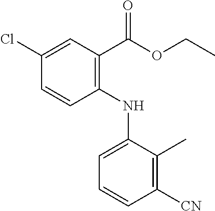 [SMILES: CCOC(=O)c1cc(Cl)ccc1Nc1cccc(C#N)c1C] | MS (m/z) 315.0 (M + H)⁺ | 3-bromo-2-meth- ylbenzonitrile | ethyl 2-amino- 5-chlorobenzoate |
| 192 | methyl 5-fluoro- 2-((4-fluoro-2- methylphenyl)- amino)-4-(tri- fluoromethoxy)- benzoate | 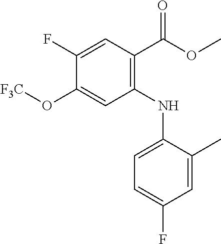 [SMILES: COC(=O)c1cc(F)c(OC(F)(F)F)cc1Nc1ccc(F)cc1C] | MS (m/z) 362.0 (M + H)⁺ | 4-fluoro-1-iodo- 2-methylbenzene | methyl 2-amino- 5-fluoro-4-(tri- fluoromethoxy)- benzoate |
| 193 | methyl 2-((5- cyano-2-methyl- phenyl)amino)- 5-(trifluorometh- yl)benzoate | 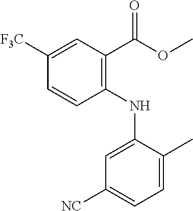 [SMILES: COC(=O)c1cc(C(F)(F)F)ccc1Nc1cc(C#N)ccc1C] | MS (m/z) 335.0 (M + H)⁺ | 3-bromo-4-meth- ylbenzonitrile | methyl 2-amino- 5-(trifluorometh- yl)benzoate |
| 194 | methyl 2-((3- cyano-4-fluoro- 2-methylphenyl)- amino)-5-(tri- fluoromethyl)- benzoate | 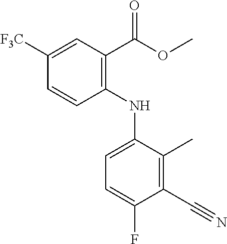 [SMILES: COC(=O)c1cc(C(F)(F)F)ccc1Nc1ccc(F)c(C#N)c1C] | MS (m/z) 351.2 (M − H)⁻ | 3-bromo-6-fluoro- 2-methylbenzo- nitrile | methyl 2-amino- 5-(trifluorometh- yl)benzoate |

### Intermediate 195

### Ethyl 2-(bicyclo[1.1.1]pentan-1-ylamino)-4-(trifluoromethyl)benzoate

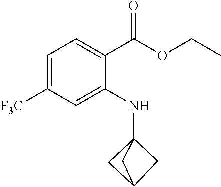 [SMILES: CCOC(=O)c1ccc(C(F)(F)F)cc1NC12CC(C1)C2]

A 100 mL sealed tube fitted with a magnetic stir-bar was charged with ethyl 2-fluoro-4-(trifluoromethyl)benzoate (2.2 g, 9.32 mmol), bicyclo[1.1.1]pentan-1-amine, 2Hydrochloride (1.744 g, 11.18 mmol), DMF (20 mL) and DIPEA (8.14 mL, 46.6 mmol). The sealed tube was heated at 80° C. for 20 hours and then cooled to RT. The reaction mixture was quenched with water (100 mL) and extracted EtOAc (2×100 mL). The combined organic extracts were washed with water (100 mL), brine (100 mL), dried over Na₂SO₄ and concentrated in vacuo. The crude product was purified by column chromatography (Isolera, 100 g SNAP column, 1% EtOAc/petroleum ether over 60 mins) to give the title compound as a colorless oil (1.1 g, 1.662 mmol, 9.55% yield). GCMS (m/z) 298.1 (M−H)⁻

### Intermediate 196

### 2-((4-Bromo-2-methylphenyl)amino)-4-(trifluoromethyl)benzonitrile

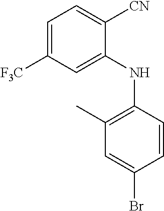 [SMILES: Cc1cc(Br)ccc1Nc1cc(C(F)(F)F)ccc1C#N]

Sodium hydride (60% wt in mineral oil) (2.068 g, 51.7 mmol) was added portionwise, over 5 minutes, to a solution of 4-bromo-2-methylaniline (5.90 g, 31.7 mmol) in DMSO (23.19 ml) under N₂ and the reaction was stirred at RT for 2 hours. The reaction mixture was cooled in an ice bath and treated with a solution of 2-fluoro-4-(trifluoromethyl)benzonitrile (3 g, 15.86 mmol) in DMSO (11.60 ml) over 5 minutes. The reaction mixture was stirred at RT for 1.5 hour and then cooled in an ice bath. Saturated NH₄Cl (˜140 mL) was added slowly followed by EtOAc. Layers were separated, and the organic layer was dried over Na₂SO₄, filtered and concentrated. The residue was dissolved in DCM (15 mL) and purified by column chromatography (ISCO, 220 g column, heptane: 3 minutes; 0-20% EtOAc/heptane over 20 minutes) to give the title compound as a white solid (3.32 g, 9.35 mmol, 58.9% yield). MS (m/z) 355.1 (M+H)⁺.

Intermediates 197-211 were prepared from the indicated benzonitrile and aniline by methods analogous to those described for Intermediate 196.

| Int. | Name | Structure | Characterization | Benzonitrile | Aniline |
|---|---|---|---|---|---|
| 197 | 4-bromo-2- ((4-fluoro-2- methylphenyl) amino)benzo- nitrile | 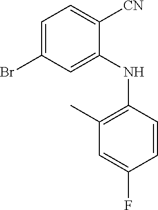 [SMILES: Cc1cc(F)ccc1Nc1cc(Br)ccc1C#N] | ¹H NMR (400 MHz, DMSO-d₆) δ: 8.29 (s, 1H), 7.54 (d, J = 8.31 Hz, 1H), 7.18-7.25 (m, 2H), 7.09 (td, J = 8.52, 3.20 Hz, 1H), 6.97 (dd, J = 8.31, 1.96 Hz, 1H), 6.48 (d, J = 1.71 Hz, 1H), 2.14 (s, 3H) | 4-bromo-2- fluorobenzo- nitrile | 4-fluoro- 2- methyl- aniline |
| 198 | 2-((2,4- difluorophenyl) amino)-4- (trifluoromethyl) benzonitrile | 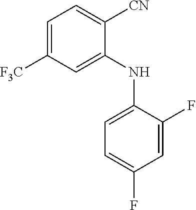 [SMILES: N#Cc1ccc(C(F)(F)F)cc1Nc1ccc(F)cc1F] | MS (m/z) 299.2 (M + H)⁺ | 2-fluoro-4- (trifluoromethyl) benzonitrile | 2,4- difluoro- aniline |
| 199 | 2-((4- ethoxyphenyl) amino)-4- (trifluoromethyl) benzonitrile | 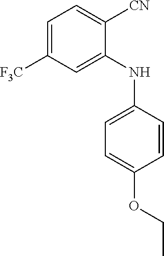 [SMILES: CCOc1ccc(Nc2cc(C(F)(F)F)ccc2C#N)cc1] | MS (m/z) 306.9 (M + H)⁺ | 2-fluoro-4- (trifluoromethyl) benzonitrile | 4- ethoxy- aniline |
| 200 | 2-((4-fluoro-2- methylphenyl) amino)-4- (trifluoromethyl) benzonitrile | 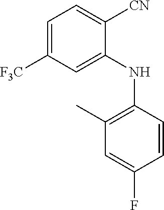 [SMILES: Cc1cc(F)ccc1Nc1cc(C(F)(F)F)ccc1C#N] | MS (m/z) 295.2 (M + H)⁺ | 2-fluoro-4- (trifluoromethyl) benzonitrile | 4-fluoro- 2- methyl- aniline |
| 201 | 2-((4- fluorophenyl) amino)-4- (trifluoromethyl) benzonitrile | 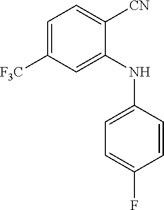 [SMILES: N#Cc1ccc(C(F)(F)F)cc1Nc1ccc(F)cc1] | ¹H NMR (400 MHz, DMSO-d₆) δ: 8.82 (s, 1H), 7.88 (d, J = 8.03 Hz, 1H), 7.28-7.18 (m, 6H) | 2-fluoro-4- (trifluoromethyl) benzonitrile | 4- fluoro- aniline |
| 202 | Cis-rac-2- (((3R,4R)-3- methyl- tetrahydro- 2H-pyran-4- yl)amino)-4- (trifluoromethyl) benzonitrile | 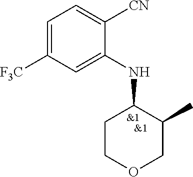 [SMILES: C[C@H]1COCC[C@H]1Nc1cc(C(F)(F)F)ccc1C#N] | MS (m/z) 285 (M + H)⁺ | 2-fluoro-4- (trifluoromethyl) benzonitrile | Cis-rac- 3-methyl- tetrahydro- 2H- pyran-4- amine, Hydro- chloride |
| 203 | Trans-rac-2- (((3R,4S)-3- methyl- tetrahydro-2H- pyran-4- yl)amino)-4- (trifluoromethyl) benzonitrile | 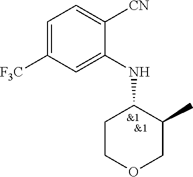 [SMILES: C[C@H]1COCC[C@@H]1Nc1cc(C(F)(F)F)ccc1C#N] | MS (m/z) 285 (M + H)⁺ | 2-fluoro-4- (trifluoromethyl) benzonitrile | Trans-3- methyl- tetrahydro- 2H- pyran-4- amine |
| 204 | 5-chloro-4- (difluoromethoxy)- 2-((4- fluoro-2- methylphenyl) amino)benzo- nitrile | 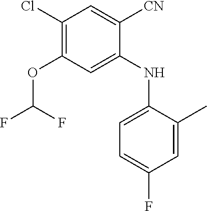 [SMILES: Cc1cc(F)ccc1Nc1cc(OC(F)F)c(Cl)cc1C#N] | ¹H NMR (400 MHz, DMSO-d₆) δ: 8.50-8.39- (m, 1H), 8.04-7.86 (m, 1H), 7.31-6.98 (m, 3H), 6.06 (d, J = 11.74 Hz, 1H), 2.20- 2.10 (m, 3H) | 5-chloro-4- (difluoro- methoxy)-2- fluorobenzo- nitrile | 4-fluoro- 2- methyl- aniline |
| 205 | 2-fluoro-6-((4- fluoro-2- methylphenyl) amino)benzo- nitrile | 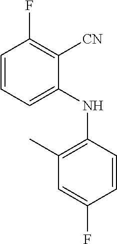 [SMILES: Cc1cc(F)ccc1Nc1cccc(F)c1C#N] | MS (m/z) 245.2 (M + H)⁺ | 2,6- difluoro- benzonitrile | 4-fluoro- 2- methyl- aniline |
| 206 | 2-((2- cyclopropyl- 4- fluorophenyl) amino)benzo- nitrile | 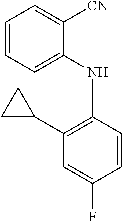 [SMILES: N#Cc1ccccc1Nc1ccc(F)cc1C1CC1] | MS (m/z) 253.2 (M + H)⁺ | 2- fluoro- benzonitrile | 2- cyclopropyl- 4- fluoro- aniline |
| 207 | 2-((2-ethyl-4- fluorophenyl) amino)-4- (trifluoromethyl) yl)benzonitrile | 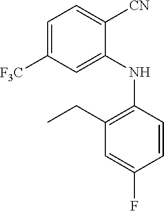 [SMILES: CCc1cc(F)ccc1Nc1cc(C(F)(F)F)ccc1C#N] | MS (m/z) 309.1 (M + H)⁺ | 2-fluoro-4- (trifluoromethyl) benzonitrile | 2-ethyl- 4- fluoro- aniline |
| 208 | 2-((4-fluoro-2- methylphenyl) amino)-5- (trifluoromethoxy) benzonitrile | 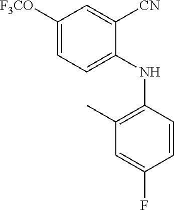 [SMILES: Cc1cc(F)ccc1Nc1ccc(OC(F)(F)F)cc1C#N] | ¹H NMR (400 MHz, DMSO-d₆) δ: 8.24 (s, 1H), 7.73 (d, J = 2.9 Hz, 1H), 7.41 (dd, J = 2.0, 9.3 Hz, 1H), 7.22-7.18 (m, 2H), 7.10-7.05 (m, 1H), 6.46 (d, J = 9.3 Hz, 1H), 2.14 (s, 3H) | 2-fluoro-5- (trifluoromethoxy) benzonitrile | 4-fluoro- 2- methyl- aniline |
| 209 | 2-((2-bromo- 4- fluorophenyl) amino)-5- chlorobenzo- nitrile | 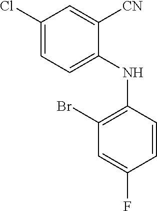 [SMILES: N#Cc1cc(Cl)ccc1Nc1ccc(F)cc1Br] | ¹H NMR (400 MHz, DMSO-d₆) δ: 8.42 (s, 1H), 7.77 (d, J = 2.9 Hz, 1H), 7.70 (dd, J = 2.9, 8.3 Hz, 1H), 7.48 (dd, J = 2.7, 9.0 Hz, 1H), 7.39- 7.27 (m, 2H), 6.58 (d, J = 9.3 Hz, 1H) | 5-chloro-2- fluoro- benzonitrile | 2- bromo-4- fluoro- aniline |
| 210 | 2-((4-fluoro-2- methylphenyl) amino)-4- (trifluoromethoxy) benzonitrile | 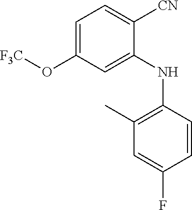 [SMILES: Cc1cc(F)ccc1Nc1cc(OC(F)(F)F)ccc1C#N] | ¹H NMR (400 MHz, CHLOROFORM-d) δ: 7.53 (d, J = 8.3 Hz, 1H), 7.21 (dd, J = 8.8, 5.4 Hz, 1H), 7.07 (dd, J = 9.3, 2.9 Hz, 1H), 7.00 (td, J = 8.3, 2.9 Hz, 1H), 6.70-6.60 (m, 1H), 6.30 (d, J = 1.5 Hz, 1H), 6.18 (br s, 1H), 2.26 (s, 3H) | 2-fluoro-4- (trifluoromethoxy) benzonitrile | 4-fluoro- 2- methyl- aniline |
| 211 | 2- (benzylamino)- 4- (trifluoromethyl) benzonitrile | 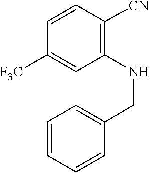 [SMILES: N#Cc1ccc(C(F)(F)F)cc1NCc1ccccc1] | ¹H NMR (400 MHz, DMSO-d₆) δ: 7.71 (d, J = 8 Hz, 1H), 7.42-7.31 (m, 5H), 7.26-7.22, m, 1H), 6.89-6.85 (m, 2H), 4.52 (d, J = 6 Hz, 2H). | 2-fluoro-4- (trifluoromethyl) benzonitrile | Phenyl- methanamine |

### Intermediate 212

### 2-((2-Methylcyclohexyl)amino)-4-(trifluoromethyl)benzonitrile

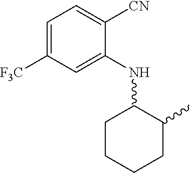 [SMILES: CC1CCCCC1Nc1cc(C(F)(F)F)ccc1C#N]

2-Fluoro-4-(trifluoromethyl)benzonitrile (2.209 mL, 15.86 mmol) and 2-methylcyclohexan-1-amine (10.45 mL, 79 mmol) were dissolved in acetonitrile (50 mL) and heated to 80° C. for 18 hr. The mixture was concentrated and purified by column chromatography (220 g column, 0-5% EtOAc/heptane) to give the title compound as an off-white solid (3.9 g, 13.8 mmol, 87%). MS (m/z) 283.2 (M+H)⁺.

Intermediates 213-215 were prepared from the indicated benzonitrile and aniline by methods analogous to those described for Intermediate 212

| Int. | Name | Structure | Characterization | Benzonitrile | Aniline |
|---|---|---|---|---|---|
| 213 | 2-(((1S,2R)-2- methylcyclo- hexylamino)- 4-(trifluoro- methyl)benzo- nitrile | 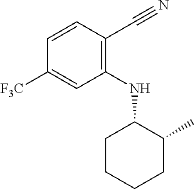 [SMILES: C[C@@H]1CCCC[C@@H]1Nc1cc(C(F)(F)F)ccc1C#N] | MS (m/z) 283.2 (M + H)⁺ | 2-fluoro-4-(tri- fluoromethyl)- benzonitrile | (1S,2R)-2-methyl- cyclohexan-1-amine, Hydrochloride |
| 214 | 2-(((1R,2S)-2- methylcyclo- hexyl)amino)- 4-(trifluoro- methyl)benzo- nitrile | 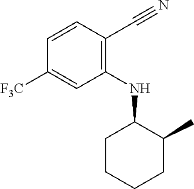 [SMILES: C[C@H]1CCCC[C@H]1Nc1cc(C(F)(F)F)ccc1C#N] | MS (m/z) 283.2 (M + H)⁺ | 2-fluoro-4-(tri- fluoromethyl)- benzonitrile | (1R,2S)-2-methyl- cyclohexan-1-amine, Hydrochloride |
| 215 | 2-((3-fluoro- cyclopentyl)- amino)-4- (trifluoro- methyl)benzo- nitrile | 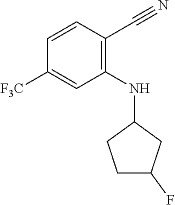 [SMILES: N#Cc1ccc(C(F)(F)F)cc1NC1CCC(F)C1] | MS (m/z) 273.2 (M + H)⁺ | 2-fluoro-4-(tri- fluoromethyl)- benzonitrile | 3-fluorocyclopentan- 1-amine, Hydro- chloride |

### Intermediate 216

### 2-((4-Fluoro-2-methylphenyl)amino)-4-(trifluoromethyl)benzoic Acid

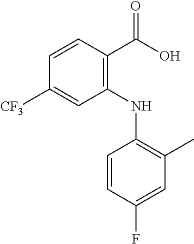 [SMILES: Cc1cc(F)ccc1Nc1cc(C(F)(F)F)ccc1C(=O)O]

LiOH.H₂O (2.434 g, 58.0 mmol) was added to a stirring solution of ethyl 2-((4-fluoro-2-methylphenyl)amino)-4-(trifluoromethyl)benzoate (3.3 g, 9.67 mmol) in THF (20 mL) and water (6.67 mL) at 30° C. under nitrogen. The reaction mixture was stirred at 60° C. for 3 hours. Solvents were evaporated under reduced pressure and the resultant off-white solid was acidified to pH=2 using 1.5 N HCl. The solid was collected by filtration, washed with water and dried in vacuo. The solid was then dissolved in DCM (10 mL) and concentrated to give the title compound as a yellow solid (3 g, 9.43 mmol, 98% yield). MS (m/z) 311.9 (M−H)⁻

Intermediates 217-326 were prepared from the indicated ester by methods analogous to those described for Intermediate 216.

| Int. | Name | Structure | Characterization | Ester |
|---|---|---|---|---|
| 217 | 2-((4-fluoro-2,6- dimethylphenyl)- amino)-4- (trifluoromethyl) benzoic acid | 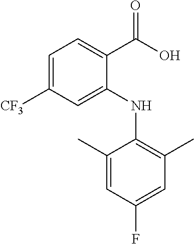 [SMILES: Cc1cc(F)cc(C)c1Nc1cc(C(F)(F)F)ccc1C(=O)O] | MS (m/z) 328.0 (M + H)⁺ | ethyl 2-((4-fluoro- 2,6-dimethylphenyl)- amino)-4-(trifluoro- methyl)benzoate |
| 218 | 2-((2-chloro-4- fluorophenyl)- amino)-4- (trifluoromethyl) benzoic acid | 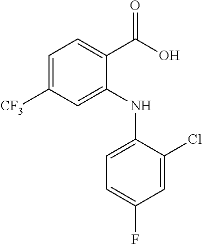 [SMILES: O=C(O)c1ccc(C(F)(F)F)cc1Nc1ccc(F)cc1Cl] | MS (m/z) 331.9 (M + H)⁺ | ethyl 2-((2-chloro-4- fluorophenyl)amino)- 4-(trifluoromethyl)- benzoate |
| 219 | 2-((4-fluoro-2- (2-methoxy- ethoxy)phenyl)- amino)-4- (trifluoromethyl) benzoic acid | 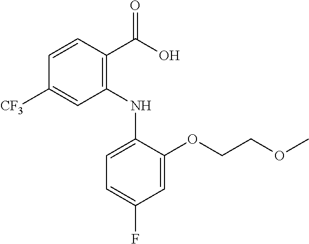 [SMILES: COCCOc1cc(F)ccc1Nc1cc(C(F)(F)F)ccc1C(=O)O] | MS (m/z) 374.0 (M + H)⁺ | ethyl 2-((4-fluoro-2- (2-methoxyethoxy)- phenyl)amino)-4- (trifluoromethyl)- benzoate |
| 220 | 2-((2,4-difluoro- phenyl)amino)-4- (trifluoromethyl) benzoic acid | 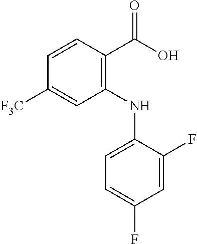 [SMILES: O=C(O)c1ccc(C(F)(F)F)cc1Nc1ccc(F)cc1F] | MS (m/z) 315.9 (M + H)⁺ | ethyl 2-((2,4-difluoro- phenyl)amino)-4- (trifluoromethyl)- benzoate |
| 221 | 2-((2-methyl- pyridin-3-yl)- amino)-4- (trifluoromethyl) benzoic acid | 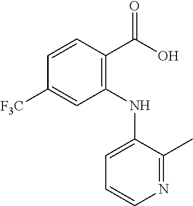 | MS (m/z) 397.0 (M + H)⁺ | ethyl 2-((2-methyl- pyridin-3-yl)amino)- 4-(trifluoromethyl)- benzoate |
| 222 | 2-((4-fluoro-2- isopropylphenyl)- amino)-4- (trifluoromethyl) benzoic acid | 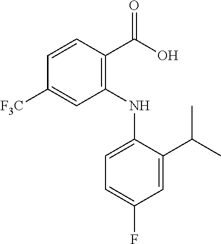 [SMILES: CC(C)c1cc(F)ccc1Nc1cc(C(F)(F)F)ccc1C(=O)O] | MS (m/z) 342.0 (M + H)⁺ | ethyl 2-((4-fluoro-2- isopropylphenyl)- amino)-4-(trifluoro- methyl)benzoate |
| 223 | 5-chloro-2-((4- fluoro-2-methyl- phenyl)amino)- benzoic acid | 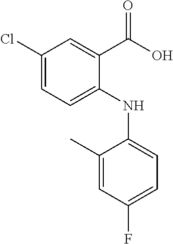 [SMILES: Cc1cc(F)ccc1Nc1ccc(Cl)cc1C(=O)O] | MS (m/z) 280.0 (M + H)⁺ | ethyl 5-chloro-2-((4- fluoro-2-methyl- phenyl)amino)- benzoate |
| 224 | 2-((4-fluoro- phenyl)amino)- benzoic acid | 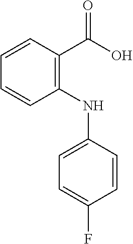 [SMILES: O=C(O)c1ccccc1Nc1ccc(F)cc1] | MS (m/z) 232.0 (M + H)⁺ | methyl 2-((4-fluoro- phenyl)amino)- benzoate |
| 225 | 2-(o-tolylamino)- benzoic acid | 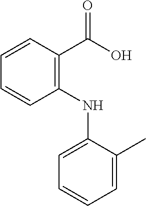 [SMILES: Cc1ccccc1Nc1ccccc1C(=O)O] | MS (m/z) 228.0 (M + H)⁺ | methyl 2-(o-tolyl- amino)benzoate |
| 226 | 2-((4-fluoro-2- methylphenyl)- amino)-5-methyl- benzoic acid | 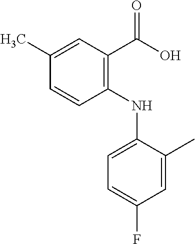 | MS (m/z) 260.0 (M + H)⁺ | ethyl 2-((4-fluoro-2- methylphenyl)amino)- 5-methylbenzoate |
| 227 | 2-((4-fluoro-2- methylphenyl)- amino)benzoic acid | 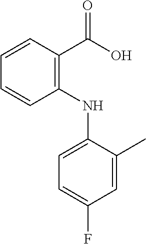 [SMILES: Cc1cc(F)ccc1Nc1ccccc1C(=O)O] | MS (m/z) 246.0 (M + H)⁺ | methyl 2-((4-fluoro-2- methylphenyl)amino)- benzoate |
| 228 | 2-((4-fluoro-2- methylphenyl)- amino)-6- (trifluoromethyl) nicotinic acid | 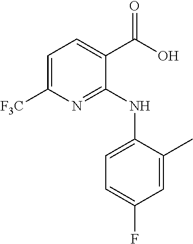 [SMILES: Cc1cc(F)ccc1Nc1nc(C(F)(F)F)ccc1C(=O)O] | MS (m/z) 315.1 (M + H)⁺ | ethyl 2-((4-fluoro-2- methylphenyl)amino)- 6-(trifluoromethyl)- nicotinate |
| 229 | 2-((4-fluoro-2- methylphenyl)- amino)-5- (trifluoromethyl) benzoic acid | 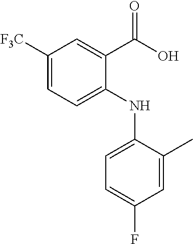 [SMILES: Cc1cc(F)ccc1Nc1ccc(C(F)(F)F)cc1C(=O)O] | MS (m/z) 312.0 (M − H)⁻ | ethyl 2-((4-fluoro-2- methylphenyl)amino)- 5-(trifluoromethyl)- benzoate |

### Intermediate 229

### 2-((4-fluoro-2-methylphenyl)amino)-5-(trifluoromethyl)benzoic Acid

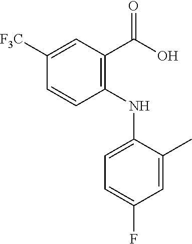 [SMILES: Cc1cc(F)ccc1Nc1ccc(C(F)(F)F)cc1C(=O)O]

To a solution of methyl 2-((4-fluoro-2-methylphenyl)amino)-5-(trifluoromethyl)benzoate (2.3 g, 7.03 mmol) in THF (20 mL) and water (10 mL) under nitrogen was added LiOH (1.68 g, 70.3 mmol). The reaction mixture was stirred at 80° C. for 4 h. The reaction mixture was cooled to RT and concentrated under vacuum. Crude material was extracted with 100 mL DCM and washed with 50 mL water. Aqueous layer was acidified with 1.5 N HCl 20 mL and extracted with DCM (100 mL) twice. Combined organic layers were dried over sodium sulphate, filtered and concentrated under vacuum to afford the title compound as a yellow solid (2.1 g, 6.7 mmol, 95% yield). MS (m/z) 314.0 (M+H)⁺.

| | | | | |
|---|---|---|---|---|
| 230 | 2-((4-fluoro-2- methoxyphenyl)- amino)-4- (trifluoromethyl) benzoic acid | 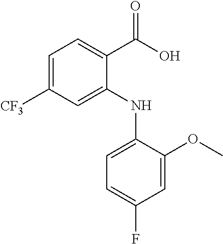 [SMILES: COc1cc(F)ccc1Nc1cc(C(F)(F)F)ccc1C(=O)O] | MS (m/z) 330.0 (M + H)⁺ | ethyl 2-((4-fluoro-2- methoxyphenyl)- amino)-4-(trifluoro- methyl)benzoate |
| 231 | 2-((4-fluoro-2- methylphenyl)- amino)-6- (trifluoromethyl) benzoic acid | 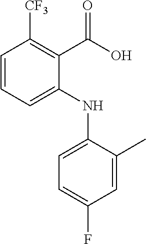 [SMILES: Cc1cc(F)ccc1Nc1cccc(C(F)(F)F)c1C(=O)O] | MS (m/z) 314.0 (M + H)⁺ | ethyl 2-((4-fluoro-2- methylphenyl)- amino)-6-(trifluoro- methyl)benzoate |
| 232 | 2-((4-fluoro-2- methoxyphenyl)- amino)-6- (trifluoromethyl) benzoic acid | 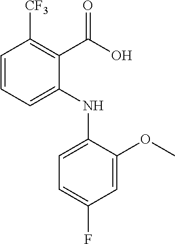 [SMILES: COc1cc(F)ccc1Nc1cccc(C(F)(F)F)c1C(=O)O] | MS (m/z) 330.0 (M + H)⁺ | ethyl 2-((4-fluoro-2- methoxyphenyl)- amino)-6-(trifluoro- methyl)benzoate |
| 233 | 2-((4-fluoro-2- methylphenyl)- amino)-4- (trifluorometh- oxy)benzoic acid | 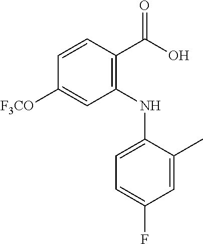 [SMILES: Cc1cc(F)ccc1Nc1cc(OC(F)(F)F)ccc1C(=O)O] | MS (m/z) 330.0 (M + H)⁺ | methyl 2-((4-fluoro- 2-methylphenyl)- amino)-4-(trifluoro- methoxy)benzoate |
| 234 | 4-chloro-2-((4- fluoro-2-methyl- phenyl)amino)- benzoic acid | 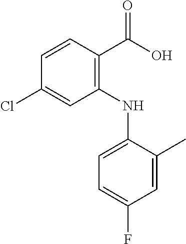 [SMILES: Cc1cc(F)ccc1Nc1cc(Cl)ccc1C(=O)O] | MS (m/z) 280.0 (M + H)⁺ | ethyl 4-chloro-2-((4- fluoro-2-methyl- phenyl)amino)- benzoate |
| 235 | 2-((4-fluoro-2- methylphenyl)- amino)-5- (trifluoromethyl) benzoic acid | 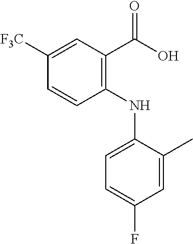 [SMILES: Cc1cc(F)ccc1Nc1ccc(C(F)(F)F)cc1C(=O)O] | MS (m/z) 314.0 (M + H)⁺ | methyl 2-((4-fluoro- 2-methylphenyl)- amino)-5-(trifluoro- methyl)benzoate |
| 236 | 4,5-difluoro-2- ((4-fluoro-2- methylphenyl)- amino)benzoic acid | 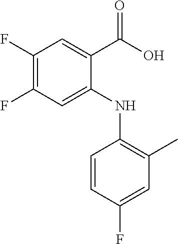 [SMILES: Cc1cc(F)ccc1Nc1cc(F)c(F)cc1C(=O)O] | MS (m/z) 282.1 (M + H)⁺ | methyl 4,5-difluoro- 2-((4-fluoro-2- methylphenyl)- amino)benzoate |
| 237 | 4,5-difluoro-2- ((4-fluoro-2- isopropylphenyl)- amino)benzoic acid | 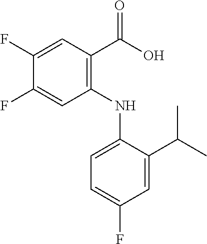 [SMILES: CC(C)c1cc(F)ccc1Nc1cc(F)c(F)cc1C(=O)O] | MS (m/z) 310.1 (M + H)⁺ | methyl 4,5-difluoro- 2-((4-fluoro-2- isopropylphenyl)- amino)benzoate |
| 238 | 4-cyano-2-((4- fluoro-2- isopropylphenyl)- amino)benzoic acid | 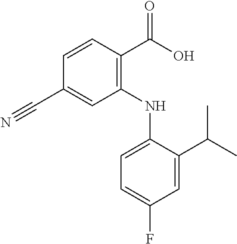 [SMILES: CC(C)c1cc(F)ccc1Nc1cc(C#N)ccc1C(=O)O] | MS (m/z) 299.2 (M + H)⁺ | methyl 4-cyano-2- ((4-fluoro-2- isopropylphenyl)- amino)benzoate |
| 239 | 2-((2-ethyl-4- fluorophenyl)- amino)-5- (trifluoromethyl) benzoic acid | 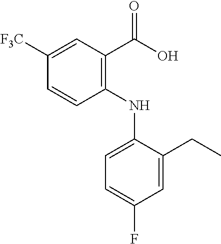 [SMILES: CCc1cc(F)ccc1Nc1ccc(C(F)(F)F)cc1C(=O)O] | MS (m/z) 328.0 (M + H)⁺ | methyl 2-((2-ethyl- 4-fluorophenyl)- amino)-5-(trifluoro- methyl)benzoate |
| 240 | 2-((4-methyl- thiazol-5-yl)- amino)-5- (trifluoromethyl) benzoic acid | 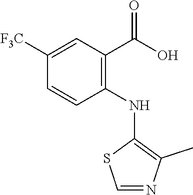 [SMILES: Cc1ncsc1Nc1ccc(C(F)(F)F)cc1C(=O)O] | MS (m/z) 303.0 (M + H)⁺ | methyl 2-((4- methylthiazol-5-yl)- amino)-5-(trifluoro- methyl)benzoate |
| 241 | 5-fluoro-2-((4- fluoro-2- methylphenyl)- amino)-6-meth- oxynicotinic acid | 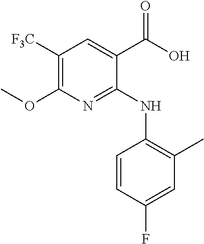 | MS (m/z) 295 (M + H)⁺ | methyl 5-fluoro-2- ((4-fluoro-2-methyl- phenyl)amino)-6- methoxynicotinate |
| 242 | 5-cyano-2-((4- fluoro-2-methyl- phenyl)amino)- 4-(trifluorometh- yl)benzoic acid | 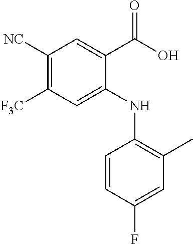 [SMILES: Cc1cc(F)ccc1Nc1cc(C(F)(F)F)c(C#N)cc1C(=O)O] | MS (m/z) 337.2 (M + H)⁺ | methyl 5-cyano-2- ((4-fluoro-2-meth- ylphenyl)amino)- 4-(trifluoromethyl)- benzoate |
| 243 | 5-chloro-4- (difluoromethyl)- 2-((4-fluoro-2- methylphenyl)- amino)benzoic acid | 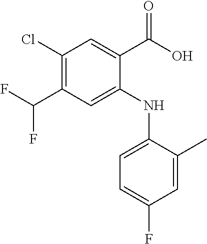 [SMILES: Cc1cc(F)ccc1Nc1cc(C(F)F)c(Cl)cc1C(=O)O] | MS (m/z) 330.2 (M + H)⁺ | methyl 5-chloro-4- (difluoromethyl)-2- ((4-fluoro-2-meth- ylphenyl)amino)- benzoate |
| 244 | 5-chloro-6-cyano- 2-((2-methyl-4- (trifluorometh- oxy)phenyl)- amino)nicotinic acid | 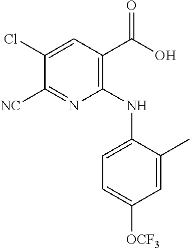 [SMILES: Cc1cc(OC(F)(F)F)ccc1Nc1nc(C#N)c(Cl)cc1C(=O)O] | MS (m/z) 372.1 (M + H)⁺ | methyl 5-chloro-6- cyano-2-((2-methyl- 4-(trifluorometh- oxy)phenyl)amino)- nicotinate |
| 245 | 5-chloro-6-cyano- 2-((4-fluoro-2- methylphenyl)- amino)nicotinic acid | 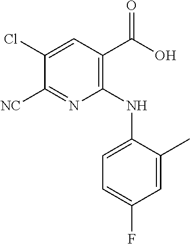 [SMILES: Cc1cc(F)ccc1Nc1nc(C#N)c(Cl)cc1C(=O)O] | MS (m/z) 306.1 (M + H)⁺ | methyl 5-chloro-6- cyano-2-((4-fluoro- 2-methylphenyl)- amino)nicotinate |
| 246 | 2-((2,4-dimeth- oxyphenyl)- amino)-5-(tri- fluoromethyl) benzoic acid | 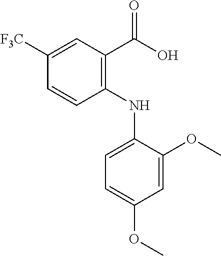 [SMILES: COc1ccc(Nc2ccc(C(F)(F)F)cc2C(=O)O)c(OC)c1] | MS (m/z) 342.1 (M + H)⁺ | ethyl 2-((2,4- dimethoxyphenyl)- amino)-5-(trifluoro- methyl)benzoate |
| 247 | 2-((4-fluoro-2- methoxyphenyl)- amino)-5- (trifluoromethyl) benzoic acid | 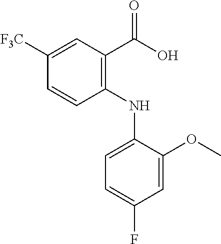 [SMILES: COc1cc(F)ccc1Nc1ccc(C(F)(F)F)cc1C(=O)O] | MS (m/z) 330.1 (M + H)⁺ | ethyl 2-((4-fluoro-2- methoxyphenyl)- amino)-5-(trifluoro- methyl)benzoate |
| 248 | 5-chloro-4-fluoro- 2-((2-methyl-4- (trifluorometh- oxy)phenyl)- amino)benzoic acid | 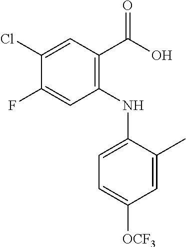 [SMILES: Cc1cc(OC(F)(F)F)ccc1Nc1cc(F)c(Cl)cc1C(=O)O] | MS (m/z) 364.0 (M + H)⁺ | methyl 5-chloro-4- fluoro-2-((2-methyl- 4-(trifluorometh- oxy)phenyl)amino)- benzoate |
| 249 | 5-chloro-4-fluoro- 2-((4-fluoro-2- methylphenyl)- amino)benzoic acid | 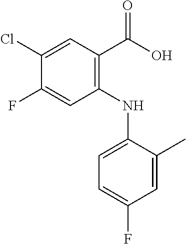 [SMILES: Cc1cc(F)ccc1Nc1cc(F)c(Cl)cc1C(=O)O] | MS (m/z) 299.1 (M + 2H)⁺ | methyl 5-chloro-4- fluoro-2-((4-fluoro- 2-methylphenyl)- amino)benzoate |
| 250 | 2-((4-fluoro-2- isopropylphenyl)- amino)-5- (trifluoromethyl) benzoic acid | 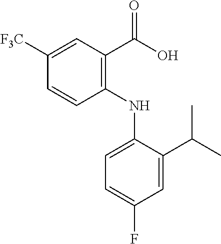 [SMILES: CC(C)c1cc(F)ccc1Nc1ccc(C(F)(F)F)cc1C(=O)O] | MS (m/z) 342.1 (M + H)⁺ | ethyl 2-((4-fluoro-2- isopropylphenyl)- amino)-5-(trifluoro- methyl)benzoate |
| 251 | 6-(difluorometh- oxy)-2-((4-fluoro- 2-methylphenyl)- amino)nicotinic acid | 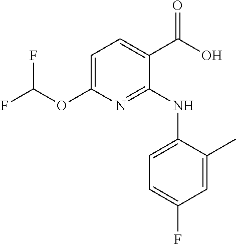 [SMILES: Cc1cc(F)ccc1Nc1nc(OC(F)F)ccc1C(=O)O] | MS (m/z) 313 (M + H)⁺ | ethyl 6-(difluoro- methoxy)-2-((4- fluoro-2-methyl- phenyl)amino)- nicotinate |
| 252 | 2-((2-(2,2,2- trifluoroethyl)- phenyl)amino)- 5-(trifluorometh- yl)benzoic acid | 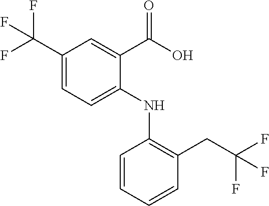 [SMILES: O=C(O)c1cc(C(F)(F)F)ccc1Nc1ccccc1CC(F)(F)F] | MS (m/z) 362 (M + H)⁺ | methyl 2-((2-(2,2,2- trifluoroethyl)- phenyl)amino)-5- (trifluoromethyl)- benzoate |
| 253 | 5-fluoro-2-((4- fluoro-2- isopropylphenyl)- amino)benzoic acid | 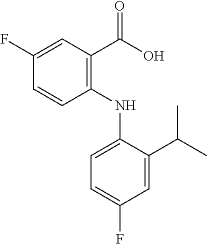 [SMILES: CC(C)c1cc(F)ccc1Nc1ccc(F)cc1C(=O)O] | MS (m/z) 292.1 (M + H)⁺ | methyl 5-fluoro-2- ((4-fluoro-2- isopropylphenyl)- amino)benzoate |
| 254 | 2-((4-(difluoro- methoxy)-2- methylphenyl)- amino)-5- (trifluoromethyl) benzoic acid | 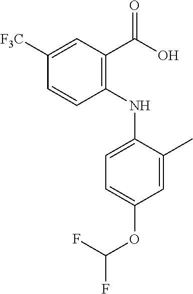 [SMILES: Cc1cc(OC(F)F)ccc1Nc1ccc(C(F)(F)F)cc1C(=O)O] | MS (m/z) 362.3 (M + H)⁺ | methyl 2-((4- (difluoromethoxy)- 2-methylphenyl)- amino)-5-(trifluoro- methyl)benzoate |
| 255 | 5-chloro-2-((2- ethyl-4-fluoro- phenyl)amino)- 4-fluorobenzoic acid | 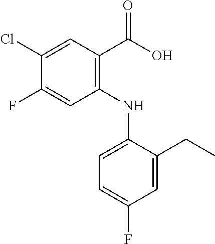 [SMILES: CCc1cc(F)ccc1Nc1cc(F)c(Cl)cc1C(=O)O] | MS (m/z) 312.1 (M + H)⁺ | methyl 5-chloro-2- ((2-ethyl-4-fluoro- phenyl)amino)-4- fluorobenzoate |
| 256 | 3-chloro-2,4- difluoro-6-((4- fluoro-2-methyl- phenyl)amino)- benzoic acid | 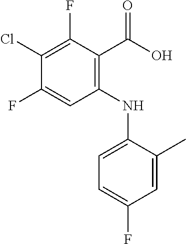 [SMILES: Cc1cc(F)ccc1Nc1cc(F)c(Cl)c(F)c1C(=O)O] | MS (m/z) 316.1 (M + H)⁺ | methyl 3-chloro-2,4- difluoro-6-((4-fluoro- 2-methylphenyl)- amino)benzoate |
| 257 | 3-chloro-4-cyano- 2-fluoro-6-((4- fluoro-2-methyl- phenyl)amino)- benzoic acid | 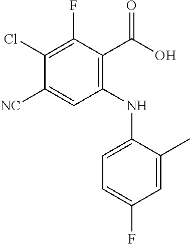 [SMILES: Cc1cc(F)ccc1Nc1cc(C#N)c(Cl)c(F)c1C(=O)O] | MS (m/z) 321.2 (M + H)⁺ | methyl 3-chloro-4- cyano-2-fluoro-6- ((4-fluoro-2-methyl- phenyl)amino)benz- oate |
| 258 | 2-((2-methyl-4- (trifluoromethyl)- phenyl)amino)-5- (trifluoromethyl) nicotinic acid | 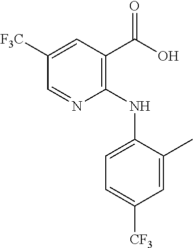 [SMILES: Cc1cc(C(F)(F)F)ccc1Nc1ncc(C(F)(F)F)cc1C(=O)O] | MS (m/z) 365.3 (M + H)⁺ | methyl 2-((2-methyl- 4-(trifluoromethyl)- phenyl)amino)-5- (trifluoromethyl)- nicotinate |
| 259 | 2-((4-chloro-2- methylphenyl)- amino)-5- (trifluoromethyl) nicotinic acid | 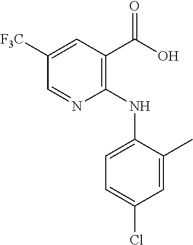 [SMILES: Cc1cc(Cl)ccc1Nc1ncc(C(F)(F)F)cc1C(=O)O] | MS (m/z) 331.1 (M + H)⁺ | methyl 2-((4-chloro- 2-methylphenyl)- amino)-5-(trifluoro- methyl)nicotinate |
| 260 | 2-((4-fluoro-2- methylphenyl)- amino)-6-methyl- nicotinic acid | 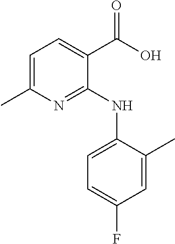 | MS (m/z) 261.3 (M + H)⁺ | methyl 2-((4-fluoro- 2-methylphenyl)- amino)-6-methyl- nicotinate |
| 261 | 5-chloro-6-cyano- 2-((2-ethyl-4- fluorophenyl)- amino)nicotinic acid | 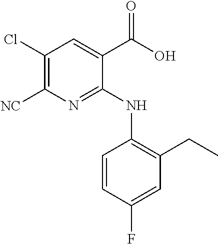 [SMILES: CCc1cc(F)ccc1Nc1nc(C#N)c(Cl)cc1C(=O)O] | MS (m/z) 320.2 (M + H)⁺ | methyl 5-chloro-6- cyano-2-((2-ethyl- 4-fluorophenyl)- amino)nicotinate |
| 262 | 2-((4-fluoro-2- isopropylphenyl)- amino)-5- (trifluoromethyl) nicotinic acid | 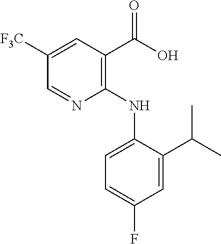 [SMILES: CC(C)c1cc(F)ccc1Nc1ncc(C(F)(F)F)cc1C(=O)O] | MS (m/z) 343.2 (M + H)⁺ | methyl 2-((4-fluoro- 2-isopropylphenyl)- amino)-5-(trifluoro- methyl)nicotinate |
| 263 | 4-(dimethyl- amino)-2-((4- fluoro-2-methyl- phenyl)amino)- benzoic acid | 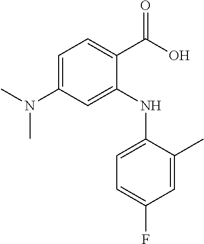 | MS (m/z) 289.0 (M + H)⁺ | methyl 4-(dimethyl- amino)-2-((4-fluoro- 2-methylphenyl)- amino)benzoate |
| 264 | 5-chloro-2-((2- ethoxy-4-fluoro- phenyl)amino)- benzoic acid | 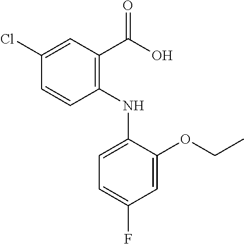 [SMILES: CCOc1cc(F)ccc1Nc1ccc(Cl)cc1C(=O)O] | MS (m/z) 310.0 (M + H)⁺ | ethyl 5-chloro-2- ((2-ethoxy-4-fluoro- phenyl)amino)- benzoate |
| 265 | 2-((4-fluoro-2- isopropylphenyl)- amino)-4-methyl- benzoic acid | 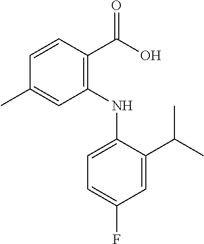 [SMILES: Cc1ccc(C(=O)O)c(Nc2ccc(F)cc2C(C)C)c1] | MS (m/z) 288.0 (M + H)⁺ | ethyl 2-((4-fluoro-2- isopropylphenyl)- amino)-4-methyl- benzoate |
| 266 | 4-(difluoro- methyl)-5-fluoro- 2-((4-fluoro-2- methylphenyl)- amino)benzoic acid | 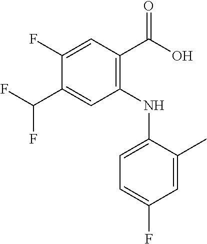 [SMILES: Cc1cc(F)ccc1Nc1cc(C(F)F)c(F)cc1C(=O)O] | MS (m/z) 314.0 (M + H)⁺ | methyl 4-(difluoro- methyl)-5-fluoro-2- ((4-fluoro-2-methyl- phenyl)amino)- benzoate |
| 267 | 5-fluoro-2-((4- fluoro-2-methyl- phenyl)amino)- 6-methylnicotinic acid | 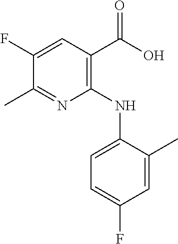 [SMILES: Cc1cc(F)ccc1Nc1nc(C)c(F)cc1C(=O)O] | MS (m/z) 279.0 (M + H)⁺ | methyl 5-fluoro-2- ((4-fluoro-2-methyl- phenyl)amino)-6- methylnicotinate |
| 268 | 5-chloro-2-((4- fluoro-2-methyl- phenyl)amino)-6- methylnicotinic acid | 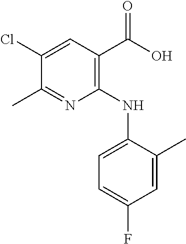 [SMILES: Cc1cc(F)ccc1Nc1nc(C)c(Cl)cc1C(=O)O] | MS (m/z) 295.0 (M + H)⁺ | methyl 5-chloro-2- ((4-fluoro-2-methyl- phenyl)amino)-6- methylnicotinate |
| 269 | 5,6-dichloro-2- ((4-fluoro-2- methylphenyl)- amino)nicotinic acid | 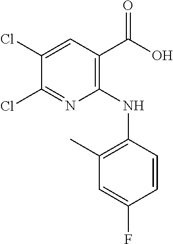 [SMILES: Cc1cc(F)ccc1Nc1nc(Cl)c(Cl)cc1C(=O)O] | MS (m/z) 317.0 (M + H)⁺ | methyl 5,6-dichloro- 2-((4-fluoro-2- methylphenyl)- amino)nicotinate |
| 270 | 3-chloro-2-fluoro- 6-((2-methyl-4- (trifluorometh- oxy)phenyl)- amino)benzoic acid | 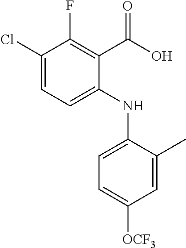 [SMILES: Cc1cc(OC(F)(F)F)ccc1Nc1ccc(Cl)c(F)c1C(=O)O] | MS (m/z) 361.9 (M − H)⁻ | methyl 3-chloro-2- fluoro-6-((2-methyl- 4-(trifluoromethoxy)- phenyl)amino)- benzoate |
| 271 | 5-cyano-2-((4- fluoro-2-methyl- phenyl)amino)- benzoic acid | 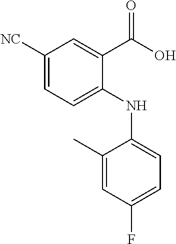 [SMILES: Cc1cc(F)ccc1Nc1ccc(C#N)cc1C(=O)O] | MS (m/z) 271.2 (M + H)⁺ | ethyl 5-cyano-2-((4- fluoro-2-methyl- phenyl)amino)- benzoate |
| 272 | 2-((4-fluoro-3- methoxy-2- methylphenyl)- amino)-4- (trifluoromethyl) benzoic acid | 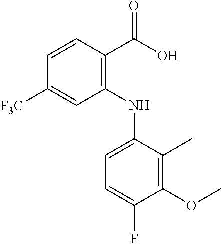 [SMILES: COc1c(F)ccc(Nc2cc(C(F)(F)F)ccc2C(=O)O)c1C] | MS (m/z) 344.0 (M + H)⁺ | ethyl 2-((4-fluoro-3- methoxy-2-methyl- phenyl)amino)-4- (trifluoromethyl)- benzoate |
| 273 | 3-chloro-2-fluoro- 6-((4-fluoro-2- methylphenyl)- amino)benzoic acid | 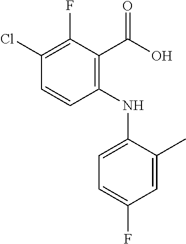 [SMILES: Cc1cc(F)ccc1Nc1ccc(Cl)c(F)c1C(=O)O] | MS (m/z) 296.0 (M − H)⁻ | methyl 3-chloro-2- fluoro-6-((4-fluoro- 2-methylphenyl)- amino)benzoate |
| 274 | 2-((3-methyl- thiophen-2-yl)- amino)-5- (trifluoromethyl) benzoic acid | 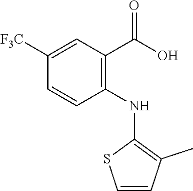 [SMILES: Cc1ccsc1Nc1ccc(C(F)(F)F)cc1C(=O)O] | MS (m/z) 302.0 (M + H)⁺ | methyl 2-((3-methyl- thiophen-2-yl)- amino)-5-(trifluoro- methyl)benzoate |
| 275 | 4-cyano-2-((4- fluoro-2-methoxy- phenyl)amino)- benzoic acid | 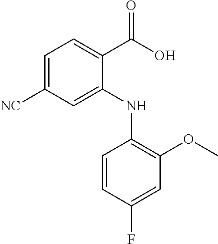 [SMILES: COc1cc(F)ccc1Nc1cc(C#N)ccc1C(=O)O] | MS (m/z) 287.0 (M + H)⁺ | ethyl 4-cyano-2-((4- fluoro-2-methoxy- phenyl)amino)- benzoate |
| 276 | 2-((4-fluoro-2- methylphenyl)- amino)-4-(meth- ylsulfonyl) benzoic acid | 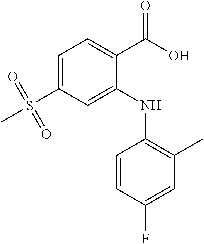 | MS (m/z) 324.0 (M + H)⁺ | ethyl 2-((4-fluoro-2- methylphenyl)- amino)-4-(methyl- sulfonyl)benzoate |
| 277 | 5-chloro-2-((2- ethyl-4-fluoro- phenyl)amino)- benzoic acid | 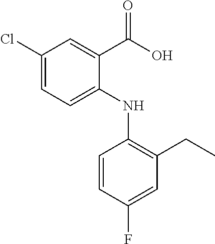 [SMILES: CCc1cc(F)ccc1Nc1ccc(Cl)cc1C(=O)O] | MS (m/z) 294.0 (M + H)⁺ | methyl 5-chloro-2- ((2-ethyl-4-fluoro- phenyl)amino) benzoate |
| 278 | 2-((4-fluoro-2- methylphenyl)- amino)-4-(2,2,2- trifluoroethoxy)- benzoic acid | 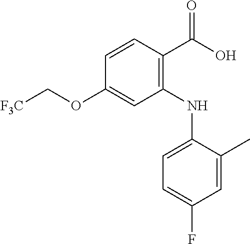 [SMILES: Cc1cc(F)ccc1Nc1cc(OCC(F)(F)F)ccc1C(=O)O] | MS (m/z) 344.0 (M + H)⁺ | ethyl 2-((4-fluoro- 2-methylphenyl)- amino)-4-(2,2,2- trifluoroethoxy)- benzoate |
| 279 | 5-fluoro-2-((4- fluoro-2-methyl- phenyl)amino)-4- methylbenzoic acid | 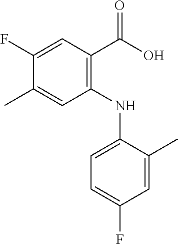 [SMILES: Cc1cc(Nc2ccc(F)cc2C)c(C(=O)O)cc1F] | MS (m/z) 278.1 (M + H)⁺ | methyl 5-fluoro-2- ((4-fluoro-2-methyl- phenyl)amino)-4- methylbenzoate |
| 280 | 2-chloro-6-((4- fluoro-2-methyl- phenyl)amino)- benzoic acid | 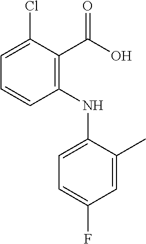 [SMILES: Cc1cc(F)ccc1Nc1cccc(Cl)c1C(=O)O] | MS (m/z) 280.0 (M + H)⁺ | ethyl 2-chloro-6- ((4-fluoro-2-methyl- phenyl)amino)- benzoate |
| 281 | 5-chloro-2-((4- fluoro-2-methoxy- phenyl)amino)- benzoic acid | 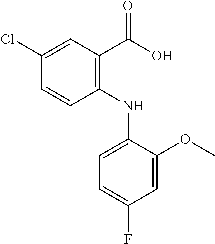 [SMILES: COc1cc(F)ccc1Nc1ccc(Cl)cc1C(=O)O] | MS (m/z) 296.0 (M + H)⁺ | methyl 5-chloro-2- ((4-fluoro-2- methoxyphenyl)- amino)benzoate |
| 282 | 4-fluoro-2-((4- fluoro-2- isopropylphenyl)- amino)benzoic acid | 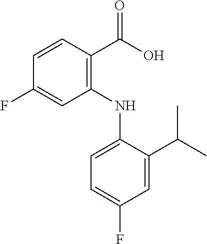 [SMILES: CC(C)c1cc(F)ccc1Nc1cc(F)ccc1C(=O)O] | MS (m/z) 290.2 (M + H)⁺ | methyl 4-fluoro-2- ((4-fluoro-2- isopropylphenyl)- amino)benzoate |
| 283 | 2-((2-(tertbutyl)- 4-fluoropheny)- amino)-5-chloro- benzoic acid | 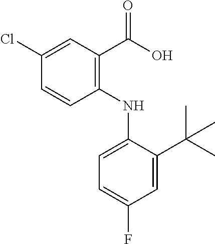 [SMILES: CC(C)(C)c1cc(F)ccc1Nc1ccc(Cl)cc1C(=O)O] | MS (m/z) 320.0 (M − H)⁻ | methyl 2-((2-tert- butyl)-4-fluoro- phenyl)amino)-5- chlorobenzoate |
| 284 | 2-((2-methyl-4- (trifluorometh- oxy)phenyl)- amino)-5- (trifluoromethyl)- benzoic acid | 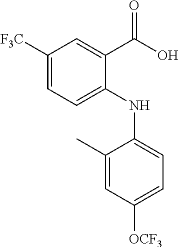 [SMILES: Cc1cc(OC(F)(F)F)ccc1Nc1ccc(C(F)(F)F)cc1C(=O)O] | MS (m/z) 379.8 (M)⁺ | methyl 2-((2-methyl- 4-(trifluorometh- oxy)phenyl)amino)- 5-(trifluoromethyl)- benzoate |
| 285 | 2-((3,4-difluoro- 2-methylphenyl)- amino)-5- (trifluoromethyl) benzoic acid | 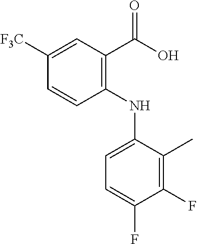 [SMILES: Cc1c(Nc2ccc(C(F)(F)F)cc2C(=O)O)ccc(F)c1F] | MS (m/z) 330.0 (M − H)⁻ | methyl 2-((3,4- difluoro-2-methyl- phenyl)amino)-5- (trifluoromethyl)- benzoate |
| 286 | 4-chloro-5-cyano- 2-((4-fluoro-2- methylphenyl)- amino)benzoic acid | 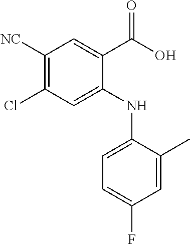 [SMILES: Cc1cc(F)ccc1Nc1cc(Cl)c(C#N)cc1C(=O)O] | MS (m/z) 303.0 (M − H)⁻ | methyl 4-chloro-5- cyano-2-((4-fluoro- 2-methylphenyl)- amino)benzoate |
| 287 | 3-chloro-6-((4- fluoro-2- isopropylphenyl)- amino)-2-methyl- benzoic acid | 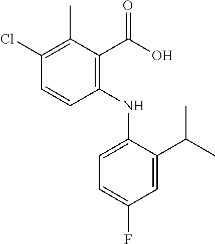 [SMILES: Cc1c(Cl)ccc(Nc2ccc(F)cc2C(C)C)c1C(=O)O] | MS (m/z) 322.0 (M + H)⁺ | methyl 3-chloro-6- ((4-fluoro-2- isopropylphenyl)- amino)-2-methyl- benzoate |
| 288 | 5-chloro-4-fluoro- 2-((4-fluoro-2- isopropylphenyl)- amino)benzoic acid | 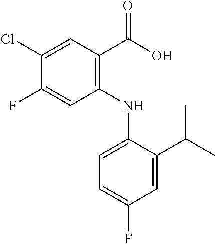 [SMILES: CC(C)c1cc(F)ccc1Nc1cc(F)c(Cl)cc1C(=O)O] | MS (m/z) 324.0 (M − H)⁻ | methyl 5-chloro-4- fluoro-2-((4-fluoro- 2-isopropylphenyl)- amino)benzoate |
| 289 | 2-((4-fluoro-2- methylphenyl)- amino)-5- methoxy-4- (trifluoromethyl) benzoic acid | 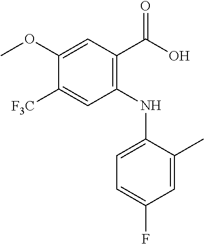 | MS (m/z) 344.0 (M + H)⁺ | ethyl 2-((4-fluoro- 2-methylphenyl)- amino)-5-methoxy- 4-(trifluoromethyl)- benzoate |
| 290 | 5-chloro-2-((4- fluoro-2-methyl- phenyl)amino)- nicotinic acid | 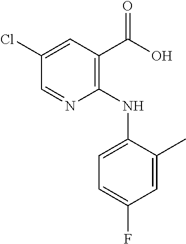 [SMILES: Cc1cc(F)ccc1Nc1ncc(Cl)cc1C(=O)O] | MS (m/z) 281.0 (M + H)⁺ | methyl 5-chloro-2- ((4-fluoro-2-methyl- phenyl)amino)- nicotinate |
| 291 | 2-((4-fluoro-2- isopropylphenyl)- amino)-4- (trifluorometh- oxy)benzoic acid | 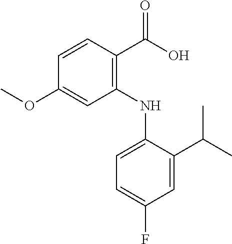 | MS (m/z) 256.0 (M − H)⁻ | methyl 2-((4-fluoro- 2-isopropylphenyl)- amino)-4-(trifluoro- methoxy)benzoate |
| 292 | 5-chloro-4-fluoro- 2-((4-fluoro-2- methylphenyl)- amino)benzoic acid | 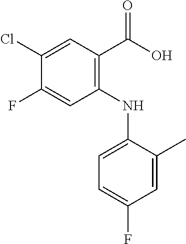 [SMILES: Cc1cc(F)ccc1Nc1cc(F)c(Cl)cc1C(=O)O] | MS (m/z) 295.8 (M − H)⁻ | methyl 5-chloro-4- fluoro-2-((4-fluoro- 2-methylphenyl)- amino)benzoate |
| 293 | 2-((4-fluoro-2- methylphenyl)- amino)-4- methoxybenzoic acid | 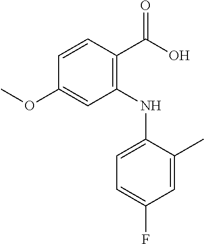 | MS (m/z) 276.0 (M + H)⁺ | ethyl 2-((4-fluoro- 2-methylphenyl)- amino)-4-methoxy- benzoate |
| 294 | 4-fluoro-2-((4- fluoro-2-methyl- phenyl)amino)- 5-(trifluoro- methyl)benzoic acid | 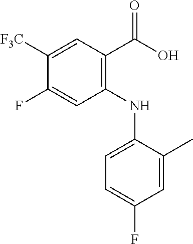 [SMILES: Cc1cc(F)ccc1Nc1cc(F)c(C(F)(F)F)cc1C(=O)O] | MS (m/z) 330.0 (M − H)⁻ | methyl 4-fluoro-2- ((4-fluoro-2-methyl- phenyl)amino)-5- (trifluoromethyl)- benzoate |
| 295 | 2-fluoro-6-((4- fluoro-2-methyl- phenyl)amino)- 3-(trifluorometh- yl)benzoic acid | 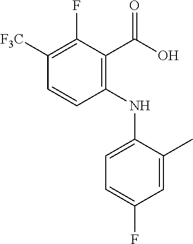 [SMILES: Cc1cc(F)ccc1Nc1ccc(C(F)(F)F)c(F)c1C(=O)O] | MS (m/z) 332.0 (M + H)⁺ | methyl 2-fluoro-6- ((4-fluoro-2-methyl- phenyl)amino)-3- (trifluoromethyl)- benzoate |
| 296 | 5-fluoro-2-((4- fluoro-2-methyl- phenyl)amino)- 4-(trifluorometh- yl)benzoic acid | 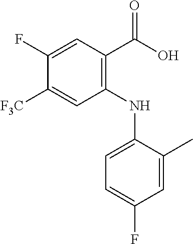 [SMILES: Cc1cc(F)ccc1Nc1cc(C(F)(F)F)c(F)cc1C(=O)O] | MS (m/z) 330.0 (M − H)⁻ | methyl 5-fluoro-2- ((4-fluoro-2-methyl- phenyl)amino)-4- (trifluoromethyl)- benzoate |
| 297 | 5-bromo-4-fluoro- 2-((4-fluoro-2- methylphenyl)- amino)benzoic acid | 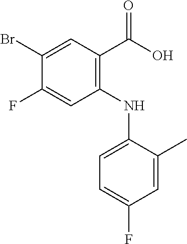 [SMILES: Cc1cc(F)ccc1Nc1cc(F)c(Br)cc1C(=O)O] | MS (m/z) 340.0 (M − H)⁻ | methyl 5-bromo-4- fluoro-2-((4-fluoro- 2-methylphenyl)- amino)benzoate |
| 298 | 5-bromo-2-((4- fluoro-2-methyl- phenyl)amino)- 4-(trifluorometh- oxy)benzoic acid | 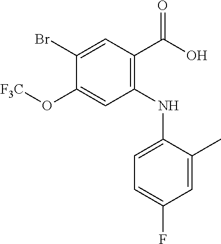 [SMILES: Cc1cc(F)ccc1Nc1cc(OC(F)(F)F)c(Br)cc1C(=O)O] | MS (m/z) 406.0 (M − H)⁻/408.0 (M + 2H)⁻ | ethyl 5-bromo-2- ((4-fluoro-2-methyl- phenyl)amino)-4- (trifluoromethoxy)- benzoate |
| 299 | 2-((4,5-difluoro- 2-methylphenyl)- amino)-5- (trifluoromethyl) benzoic acid | 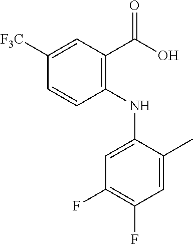 [SMILES: Cc1cc(F)c(F)cc1Nc1ccc(C(F)(F)F)cc1C(=O)O] | MS (m/z) 332.0 (M + H)⁺ | methyl 2-((4,5- difluoro-2-methyl- phenyl)amino)-5- (trifluoromethyl)- benzoate |
| 300 | 3-chloro-6-((4- fluoro-2-methyl- phenyl)amino)- 2-methylbenzoic acid | 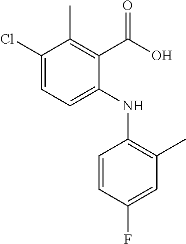 [SMILES: Cc1cc(F)ccc1Nc1ccc(Cl)c(C)c1C(=O)O] | MS (m/z) 294.2 (M + H)⁺ | methyl 3-chloro-6- ((4-fluoro-2-methyl- phenyl)amino)-2- methylbenzoate |
| 301 | 2-((2-(tertbutyl)- 4-fluorophenyl)- amino)-4- (trifluoromethyl) benzoic acid | 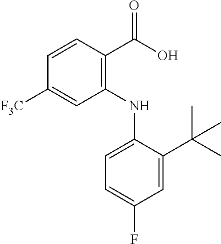 [SMILES: CC(C)(C)c1cc(F)ccc1Nc1cc(C(F)(F)F)ccc1C(=O)O] | MS (m/z) 356.2 (M + H)⁺ | ethyl 2-((2-(tertbutyl)- 4-fluorophenyl)- amino)-4-(trifluoro- methyl)benzoate |
| 302 | 2-((3,5-difluoro- 2-methylphenyl)- amino)-5- (trifluoromethyl) benzoic acid | 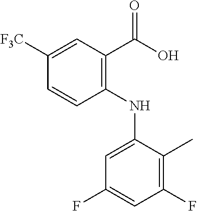 [SMILES: Cc1c(F)cc(F)cc1Nc1ccc(C(F)(F)F)cc1C(=O)O] | MS (m/z) 332.0 (M + H)⁺ | methyl 2-((3,5- difluoro-2-methyl- phenyl)amino)-5- (trifluoromethyl)- benzoate |
| 303 | 2-((4-fluoro-2- isopropylphenyl)- amino)-6-meth- oxybenzoic acid | 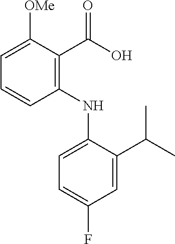 [SMILES: COc1cccc(Nc2ccc(F)cc2C(C)C)c1C(=O)O] | MS (m/z) 304.1 (M + H)⁺ | ethyl 2-((4-fluoro- 2-isopropylphenyl)- amino)-6-methoxy- benzoate |
| 304 | 2-((2-ethyl-4- fluorophenyl)- amino)-5-fluoro- benzoic acid | 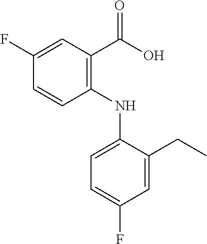 [SMILES: CCc1cc(F)ccc1Nc1ccc(F)cc1C(=O)O] | MS (m/z) 278.0 (M + H)⁺ | methyl 2-((2-ethyl- 4-fluorophenyl)- amino)-5-fluoro- benzoate |
| 305 | 5-chloro-2-((4- cyano-2-methyl- phenyl)amino)- benzoic acid | 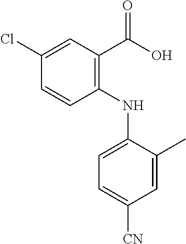 [SMILES: Cc1cc(C#N)ccc1Nc1ccc(Cl)cc1C(=O)O] | MS (m/z) 285.0 (M − H)⁻ | ethyl 5-chloro-2-((4- cyano-2-methyl- phenyl)amino)- benzoate |
| 306 | 2-((2-methyl-4- (trifluorometh- oxy)phenyl)- amino)-5- (trifluoromethyl) nicotinic acid | 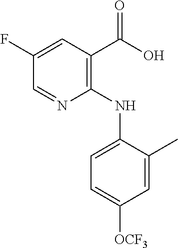 | MS (m/z) 381.0 (M + H)⁺ | methyl 2-((2-methyl- 4-(trifluoromethyl)- phenyl)amino)-5- (trifluoromethyl)- nicotinate |

### Intermediate 306

### 2-((2-methyl-4-(trifluoromethoxy)phenyl)amino)-5-(trifluoromethyl)nicotinic Acid

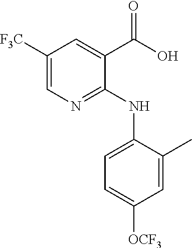 [SMILES: Cc1cc(OC(F)(F)F)ccc1Nc1ncc(C(F)(F)F)cc1C(=O)O]

To a solution containing 2-methyl-4-(trifluoromethoxy)aniline (42.4 g, 222 mmol) in water (500 mL) were added 2-chloro-5-(trifluoromethyl)nicotinic acid (50 g, 222 mmol) and PTSOH (12.65 g, 66.5 mmol) and pyridine (17.93 mL, 222 mmol). The reaction was warmed to 100° C. for 18 hr. The reaction was cooled to RT and diluted with water (1400 mL), filtered and washed with water (500 mL) and dried on vac oven at 50° C. for 18 hr to afford a crude solid. The solid was taken up in EtOAc (500 mL), dried over MgSO₄, filtered and concentrated to afford the title compound as a pale yellow solid (74.6 g, 196 mmol, 89% yield). MS (m/z) 381.1 (M+H)⁺.

| | | | | |
|---|---|---|---|---|
| 307 | 2-((2,4-difluoro- 6-methylphenyl)- amino)-5- (trifluoromethyl) benzoic acid | 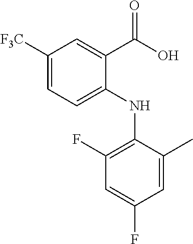 [SMILES: Cc1cc(F)cc(F)c1Nc1ccc(C(F)(F)F)cc1C(=O)O] | MS (m/z) 332.0 (M + H)⁺ | methyl 2-((2,4- difluoro-6-methyl- phenyl)amino)-5- (trifluoromethyl) benzoate |
| 308 | 6-(difluorometh- yl)-2-((4-fluoro-2- isopropylphenyl)- amino)nicotinic acid | 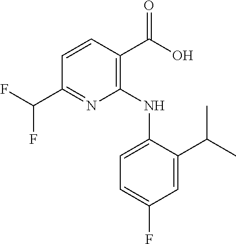 [SMILES: CC(C)c1cc(F)ccc1Nc1nc(C(F)F)ccc1C(=O)O] | MS (m/z) 325.2 (M + H)⁺ | methyl 6-(difluoro- methyl)-2-((4-fluoro- 2-isopropylphenyl)- amino)nicotinate |
| 309 | 2-((4-cyano-2- methylphenyl)- amino)-5- (trifluorometh- oxy)benzoic acid | 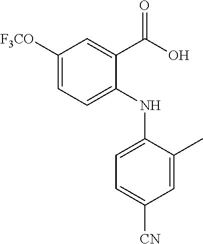 [SMILES: Cc1cc(C#N)ccc1Nc1ccc(OC(F)(F)F)cc1C(=O)O] | MS (m/z) 337.0 (M + H)⁺ | methyl 2-((4-cyano- 2-methylphenyl)- amino)-5-(trifluoro- methoxy)benzoate |
| 310 | 3-chloro-2-((4- fluoro-2-methyl- phenyl)amino)- benzoic acid | 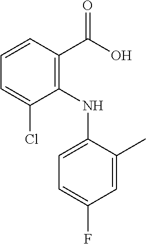 [SMILES: Cc1cc(F)ccc1Nc1c(Cl)cccc1C(=O)O] | MS (m/z) 280.0 (M + H)⁺ | ethyl 3-chloro-2- ((4-fluoro-2-methyl- phenyl)amino)- benzoate |
| 311 | 2-((4-methoxy- 2-methylphenyl)- amino)-5- (trifluoromethyl) benzoic acid | 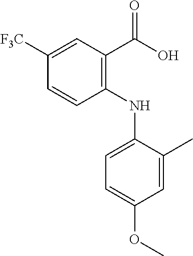 [SMILES: COc1ccc(Nc2ccc(C(F)(F)F)cc2C(=O)O)c(C)c1] | MS (m/z) 326.0 (M + H)⁺ | methyl 2-((4- methoxy-2-methyl- phenyl)amino)-5- (trifluoromethyl)- benzoate |
| 312 | 5-chloro-2-((3- cyano-2-methyl- phenyl)amino)- benzoic acid | 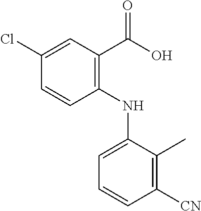 [SMILES: Cc1c(C#N)cccc1Nc1ccc(Cl)cc1C(=O)O] | MS (m/z) 285.0 (M − H)⁻ | ethyl 5-chloro-2- ((3-cyano-2-methyl- phenyl)amino)- benzoate |
| 313 | 5-fluoro-2-((4- fluoro-2-methyl- phenyl)amino)- 4-(trifluorometh- oxy)benzoic acid | 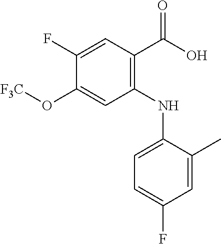 [SMILES: Cc1cc(F)ccc1Nc1cc(OC(F)(F)F)c(F)cc1C(=O)O] | MS (m/z) 346.0 (M − H)⁻ | methyl 5-fluoro-2- ((4-fluoro-2-methyl- phenyl)amino)-4- (trifluoromethoxy)- benzoate |
| 314 | 2-((5-cyano-2- methylphenyl)- amino)-5- (trifluoromethyl) benzoic acid | 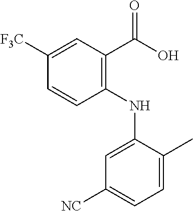 [SMILES: Cc1ccc(C#N)cc1Nc1ccc(C(F)(F)F)cc1C(=O)O] | MS (m/z) 319.2 (M − H)⁻ | methyl 2-((5-cyano- 2-methylphenyl)- amino)-5-(trifluoro- methyl)benzoate |
| 315 | 2-(difluorometh- yl)-6-((4-fluoro- 2-methylphenyl)- amino)benzoic acid | 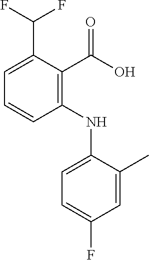 [SMILES: Cc1cc(F)ccc1Nc1cccc(C(F)F)c1C(=O)O] | MS (m/z) 296.0 (M + H)⁺ | ethyl 2-(difluoro- methyl)-6-((4-fluoro- 2-methylphenyl)- amino)benzoate |
| 316 | 3-chloro-2-fluoro- 6-((4-fluoro-2- methylphenyl)- amino)benzoic acid | 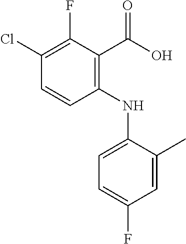 [SMILES: Cc1cc(F)ccc1Nc1ccc(Cl)c(F)c1C(=O)O] | MS (m/z) 296 (M − H)⁻ | methyl 3-chloro-2- fluoro-6-((4-fluoro- 2-methylphenyl)- amino)benzoate |
| 317 | 4,5-difluoro-2- ((4-fluoro-2- methoxyphenyl)- amino)benzoic acid | 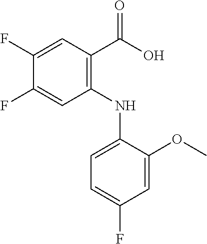 [SMILES: COc1cc(F)ccc1Nc1cc(F)c(F)cc1C(=O)O] | MS (m/z) 298 (M + H)⁺ | methyl 4,5-difluoro- 2-((4-fluoro-2-meth- oxyphenyl)amino)- benzoate |
| 318 | 5-chloro-2-((2- methoxy-4- methylphenyl)- amino)benzoic acid | 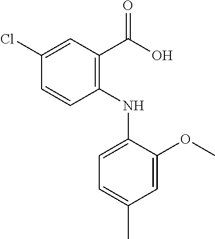 [SMILES: COc1cc(C)ccc1Nc1ccc(Cl)cc1C(=O)O] | MS (m/z) 292 (M + H)⁺ | ethyl 5-chloro-2-((2- methoxy-4-methyl- phenyl)amino)- benzoate |
| 319 | 2-((2-fluoro-6- methylphenyl)- amino)-5- (trifluoromethyl) benzoic acid | 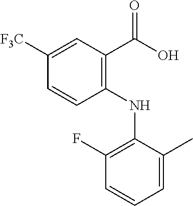 [SMILES: Cc1cccc(F)c1Nc1ccc(C(F)(F)F)cc1C(=O)O] | MS (m/z) 314 (M + H)⁺ | ethyl 2-((2-fluoro- 6-methylphenyl)- amino)-5-(trifluoro- methyl)benzoate |
| 320 | 4-(difluorometh- yl)-2-((4-fluoro-2- isopropylphenyl)- amino)benzoic acid | 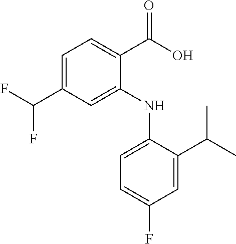 [SMILES: CC(C)c1cc(F)ccc1Nc1cc(C(F)F)ccc1C(=O)O] | MS (m/z) 324.1 (M + H)⁺ | methyl 4-(difluoro- methyl)-2-((4-fluoro- 2-isopropylphenyl)- amino)benzoate |
| 321 | 4-(difluorometh- yl)-2-((4-fluoro- 2-methylphenyl)- amino)benzoic acid | 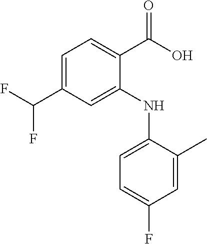 [SMILES: Cc1cc(F)ccc1Nc1cc(C(F)F)ccc1C(=O)O] | MS (m/z) 296.0 (M + H)⁺ | methyl 4-(difluoro- methyl)-2-((4-fluoro- 2-methylphenyl)- amino)benzoate |
| 322 | 2-(bicyclo[1.1.1]- pentan-1-ylamino)- 4-(trifluoromethyl) benzoic acid | 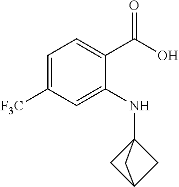 [SMILES: O=C(O)c1ccc(C(F)(F)F)cc1NC12CC(C1)C2] | MS (m/z) 272.0 (M + H)⁺ | ethyl 2-(bicyclo- [1.1.1]pentan-1-yl- amino)-4-(trifluoro- methyl)benzoate |
| 323 | 5-chloro-2-((4- fluoro-2-isopropyl- phenyl)amino)- benzoic acid | 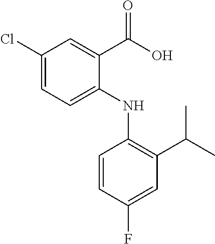 [SMILES: CC(C)c1cc(F)ccc1Nc1ccc(Cl)cc1C(=O)O] | MS (m/z) 308.0 (M + H)⁺ | methyl 5-chloro-2- ((4-fluoro-2- isopropylphenyl)- amino)benzoate |
| 324 | 2-((3-cyano-4- fluoro-2-methyl- phenyl)amino)-5- (trifluoromethyl) benzoic acid | 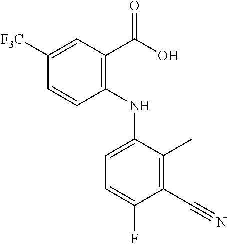 [SMILES: Cc1c(Nc2ccc(C(F)(F)F)cc2C(=O)O)ccc(F)c1C#N] | MS (m/z) 337.0 (M − H)⁻ | methyl 2-((3-cyano- 4-fluoro-2-methyl- phenyl)amino)-5- (trifluoromethyl)- benzoate |
| 325 | 5-fluoro-2-((4- fluoro-2-isopropyl- phenyl)amino)- nicotinic acid | 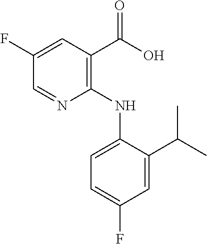 [SMILES: CC(C)c1cc(F)ccc1Nc1ncc(F)cc1C(=O)O] | MS (m/z) 293.0 (M + H)⁺ | methyl 5-fluoro-2- ((4-fluoro-2- isopropylphenyl)- amino)nicotinate |
| 326 | 5-fluoro-2-((4- fluoro-2-methyl- phenyl)amino)- benzoic acid | 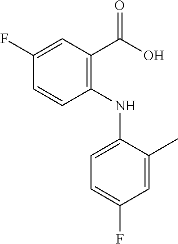 [SMILES: Cc1cc(F)ccc1Nc1ccc(F)cc1C(=O)O] | MS (m/z) 264.2 (M + H)⁺ | methyl 5-fluoro-2- ((4-fluoro-2-methyl- phenyl)amino)- benzoate |

### Intermediate 327

### 2-((4-Bromo-2-methylphenyl)amino)-4-(trifluoromethyl)benzoic Acid

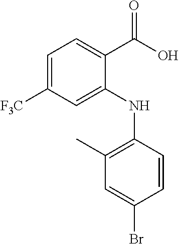 [SMILES: Cc1cc(Br)ccc1Nc1cc(C(F)(F)F)ccc1C(=O)O]

A suspension of 2-((4-bromo-2-methylphenyl)amino)-4-(trifluoromethyl)benzonitrile (1.5 g, 4.22 mmol) and potassium hydroxide (0.948 g, 16.89 mmol) in ethanol (11 ml) and water (11.00 ml) was stirred at 97° C. for 16 hours. The reaction eventually became a yellow solution. The reaction was cooled to RT and solvent was evaporated under reduced pressure. The water residue was diluted with water and adjusted to pH˜2 using 6N HCl. The solid precipitate was collected by vacuum filtration, washed with water, air dried and then placed in a vacuum oven overnight to give the title product as an off white solid (1.5771 g, 4.22 mmol, 100% yield). MS (m/z) 374.1 (M+H)⁺.

Intermediates 216 and 328-350 were prepared from the indicated benzonitrile by methods analogous to those described for Intermediate 327.

| Int. | Name | Structure | Characterization | Benzonitrile |
|---|---|---|---|---|
| 216 | 2-((4-fluoro-2- methylphenyl) amino)-4- (trifluoromethyl) benzoic acid | 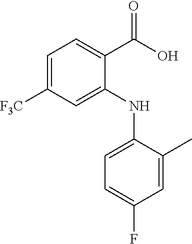 [SMILES: Cc1cc(F)ccc1Nc1cc(C(F)(F)F)ccc1C(=O)O] | MS (m/z) 314.2 (M + H)⁺. | 2-((4-fluoro-2- methylphenyl) amino)-4- (trifluoromethyl) benzonitrile |
| 328 | 2-((4- fluorophenyl) amino)-4- (trifluoromethyl) benzoic acid | 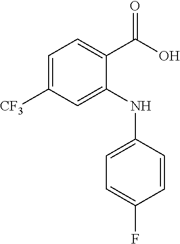 [SMILES: O=C(O)c1ccc(C(F)(F)F)cc1Nc1ccc(F)cc1] | MS (m/z) 300.1 (M + H)⁺ | 2-((4- fluorophenyl) amino)-4- (trifluoromethyl) benzonitrile |
| 329 | 4-bromo-2- ((4-fluoro-2- methylphenyl) amino)benzoic acid | 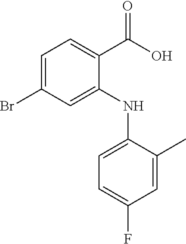 [SMILES: Cc1cc(F)ccc1Nc1cc(Br)ccc1C(=O)O] | MS (m/z) 324.1 (M + H)⁺ | 4-bromo-2- ((4-fluoro-2- methylphenyl) amino) benzonitrile |
| 330 | 2-((2,4- difluorophenyl) amino)-4- (trifluoromethyl) benzoic acid | 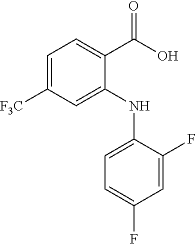 [SMILES: O=C(O)c1ccc(C(F)(F)F)cc1Nc1ccc(F)cc1F] | MS (m/z) 318.1 (M + H)⁺ | 2-((2,4- difluorophenyl) amino)-4- (trifluoromethyl) benzonitrile |
| 331 | 2-((4- ethoxyphenyl) amino)-4- (trifluoromethyl) benzoic acid | 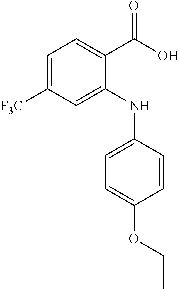 [SMILES: CCOc1ccc(Nc2cc(C(F)(F)F)ccc2C(=O)O)cc1] | MS (m/z) 326.3 (M + H)⁺ | 2-((4- ethoxyphenyl) amino)-4- (trifluoromethyl) benzonitrile |
| 332 | 2-(((1R,2S)-2- methylcyclohexyl) amino)-4- (trifluoromethyl) benzoic acid | 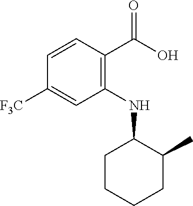 [SMILES: C[C@H]1CCCC[C@H]1Nc1cc(C(F)(F)F)ccc1C(=O)O] | MS (m/z) 302.2 (M + H)⁺ | 2-(((1R,2S)-2- methylcyclohexyl) amino)-4- (trifluoromethyl) benzonitrile |
| 333 | 2-((2- methylcyclohexyl) amino)-4- (trifluoromethyl) benzoic acid | 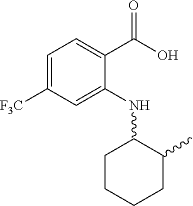 [SMILES: CC1CCCCC1Nc1cc(C(F)(F)F)ccc1C(=O)O] | MS (m/z) 302.1 (M + H)⁺ | 2-((2- methylcyclohexyl) amino)-4- (trifluoromethyl) benzonitrile |
| 334 | 2-(((1S,2R)-2- methylcyclohexyl) amino)-4- (trifluoromethyl) benzoic acid | 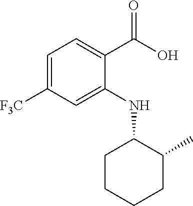 [SMILES: C[C@@H]1CCCC[C@@H]1Nc1cc(C(F)(F)F)ccc1C(=O)O] | MS (m/z) 302.2 (M + H)⁺ | 2-(((1S,2R)-2- methylcyclohexyl) amino)-4- (trifluoromethyl) benzonitrile |
| 335 | cis-rac-2- (((3R,4R)-3- methyltetrahydro- 2H-pyran-4- yl)amino)-4- (trifluoromethyl) benzoic acid | 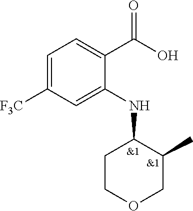 [SMILES: C[C@H]1COCC[C@H]1Nc1cc(C(F)(F)F)ccc1C(=O)O] | MS (m/z) 304 (M + H)⁺ | Cis-rac-2- (((3R,4R)-3- methyltetrahydro- 2H-pyran-4- yl)amino)-4- (trifluoromethyl) benzonitrile |
| 336 | trans-rac-2- (((3R,4R)-3- methyltetrahydro- 2H-pyran-4- yl)amino)-4- (trifluoromethyl) benzoic acid | 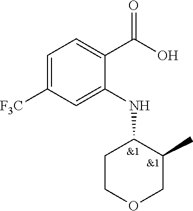 [SMILES: C[C@H]1COCC[C@@H]1Nc1cc(C(F)(F)F)ccc1C(=O)O] | MS (m/z) 304 (M + H)⁺ | Trans-rac-2- (((3R,4R)-3- methyltetrahydro- 2H-pyran-4- yl)amino)-4- (trifluoromethyl) benzonitrile |
| 337 | 5-chloro-4- (difluoromethoxy)- 2-((4- fluoro-2- methylphenyl) amino)benzoic acid | 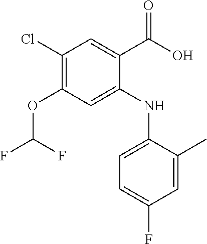 [SMILES: Cc1cc(F)ccc1Nc1cc(OC(F)F)c(Cl)cc1C(=O)O] | MS (m/z) 346.2 (M + H)⁺ | 5-chloro-4- (difluoromethoxy)- 2-((4- fluoro-2- methylphenyl)amino) benzonitrile |
| 338 | 2-((2- cyclopropyl-4- fluorophenyl) amino)-4- (trifluoromethyl) benzoic acid | 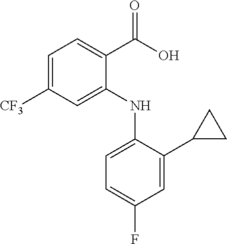 [SMILES: O=C(O)c1ccc(C(F)(F)F)cc1Nc1ccc(F)cc1C1CC1] | MS (m/z) 340.1 (M + H)⁺ | 2-((2- cyclopropyl-4- fluorophenyl) amino)-4- (trifluoromethyl) benzonitrile |
| 339 | 2-fluoro-6-((4- fluoro-2- methylphenyl) amino)benzoic acid | 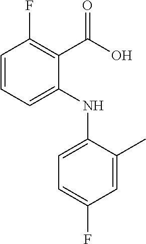 | MS (m/z) 364.1 (M + H)⁺ | 2-fluoro-6-((4- fluoro-2- methylphenyl) amino) benzonitrile |
| 340 | 2-((2- cyclopropyl-4- fluorophenyl) amino) benzoic acid | 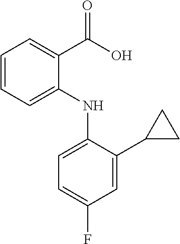 [SMILES: O=C(O)c1ccccc1Nc1ccc(F)cc1C1CC1] | MS (m/z) 372.2 (M + H)⁺ | 2-((2- cyclopropyl-4- fluorophenyl) amino) benzonitrile |
| 341 | 2-((2-ethyl-4- fluorophenyl) amino)-4- (trifluoromethyl) benzoic acid | 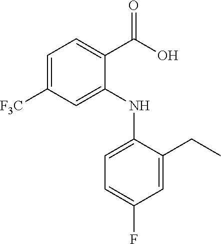 [SMILES: CCc1cc(F)ccc1Nc1cc(C(F)(F)F)ccc1C(=O)O] | MS (m/z) 328.1 (M + H)⁺ | 2-((2-ethyl-4- fluorophenyl) amino)-4- (trifluoromethyl) benzonitrile |
| 342 | 4- (difluoromethoxy)- 2-((4- fluoro-2- methylphenyl) amino)benzoic acid | 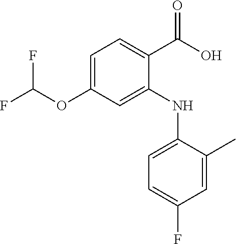 [SMILES: Cc1cc(F)ccc1Nc1cc(OC(F)F)ccc1C(=O)O] | MS (m/z) 312.1 (M + H)⁺ | 4- (difluoromethoxy)- 2-((4- fluoro-2- methylphenyl) amino) benzonitrile |
| 343 | 4-cyclopropyl- 2-((4-fluoro-2- methylphenyl) amino)benzoic acid | 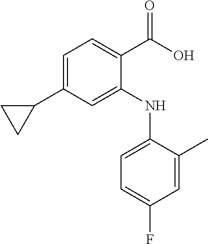 | MS (m/z) 386.2 (M + H)⁺ | 4-cyclopropyl- 2-((4-fluoro-2- methylphenyl) amino) benzonitrile |
| 344 | 2-((4-fluoro-2- methylphenyl) amino)-5- (trifluoromethoxy) benzoic acid | 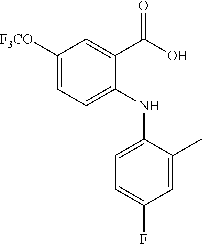 [SMILES: Cc1cc(F)ccc1Nc1ccc(OC(F)(F)F)cc1C(=O)O] | MS (m/z) 330.1 (M + H)⁺ | 2-((4-fluoro-2- methylphenyl) amino)-5- (trifluoromethoxy) benzonitrile |
| 345 | 2-((2-bromo-4- fluorophenyl) amino)-5- chlorobenzoic acid | 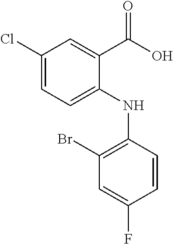 [SMILES: O=C(O)c1cc(Cl)ccc1Nc1ccc(F)cc1Br] | MS (m/z) 346.0 (M + H)⁺ | 2-((2-bromo-4- fluorophenyl) amino)-5- chlorobenzonitrile |
| 346 | 2-((4-fluoro-2- methylphenyl) amino)-4- (trifluoromethoxy) benzoic acid | 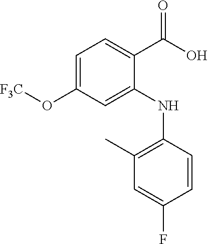 [SMILES: Cc1cc(F)ccc1Nc1cc(OC(F)(F)F)ccc1C(=O)O] | MS (m/z) 330.2 (M + H)⁺ | 2-((4-fluoro-2- methylphenyl) amino)-4- (trifluoromethoxy) benzonitrile |
| 347 | 2-((3- fluorocyclopentyl) amino)-4- (trifluoromethyl) benzoic acid | 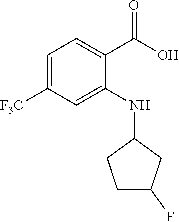 [SMILES: O=C(O)c1ccc(C(F)(F)F)cc1NC1CCC(F)C1] | MS (m/z) 392.1 (M + H)⁺ | 2-((3- fluorocyclopentyl) amino)-4- (trifluoromethyl) benzonitrile |
| 348 | 2-((4-fluoro-2- isopropylphenyl) amino)-6- (trifluoromethyl) nicotinic acid | 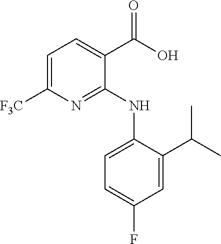 [SMILES: CC(C)c1cc(F)ccc1Nc1nc(C(F)(F)F)ccc1C(=O)O] | MS (m/z) 343.3 (M + H)⁺ | 2-((4-fluoro-2- isopropylphenyl) amino)-6- (trifluoromethyl) nicotinonitrile |
| 349 | 5-chloro-2-((4- fluoro-2- isopropylphenyl) amino)nicotinic acid | 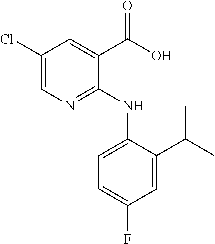 [SMILES: CC(C)c1cc(F)ccc1Nc1ncc(Cl)cc1C(=O)O] | MS (m/z) 309.1 (M + H)⁺ | 5-chloro-2-((4- fluoro-2- isopropylphenyl) amino)nicotinonitrile |
| 350 | 2- (benzylamino)- 4- (trifluoromethyl) benzoic acid | 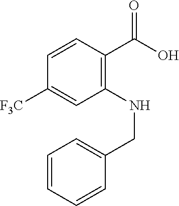 [SMILES: O=C(O)c1ccc(C(F)(F)F)cc1NCc1ccccc1] | MS (m/z) 396.0 (M + H)⁺ | 2- (benzylamino)- 4- (trifluoromethyl) benzonitrile |

### Intermediate 351

### 2-(Cyclohexylamino)-4-(trifluoromethyl)benzoic Acid

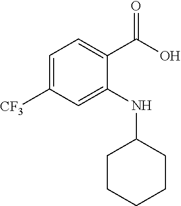 [SMILES: O=C(O)c1ccc(C(F)(F)F)cc1NC1CCCCC1]

Cyclohexanamine (1.475 g, 14.87 mmol), potassium carbonate (1.541 g, 11.15 mmol), copper (0.047 g, 0.743 mmol), and copper(II) oxide (0.030 g, 0.372 mmol) were added to a stirring solution of 2-bromo-4-(trifluoromethyl)benzoic acid (2.0 g, 7.43 mmol) in ethoxyethanol (20 mL) at 25° C. under N₂. The reaction mixture was slowly heated to 130° C. and stirred at this temperature for 24 hours. The reaction mixture was cooled to 30° C. and concentrated under reduced pressure. The resultant dark red gum was diluted with EtOAc (50 mL) and filtered through a pad of Celite. The Celite pad was washed with ethyl acetate (50 mL). The filtrate was washed with 2M HCl (50 mL), saturated NaHCO₃ (50 mL) and brine (50 mL), dried over Na₂SO₄, filtered and concentrated. The residue was purified by column chromatography (Biotage, 20 g SNAP column, 5-20% EtOAc/petroleum ether over 40 minutes) to give the title compound as an off white solid (500 mg, 1.735 mmol, 23.33% yield). MS (m/z) 288.0 (M+H)⁺.

Intermediate 352 was prepared from the indicated amine by methods analogous to those described for Intermediate 351.

| Int. | Name | Structure | Characterization | Amine |
|---|---|---|---|---|
| 352 | 2-((4- fluorobenzyl) amino)-4- (trifluoromethyl) benzoic acid | 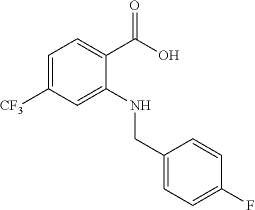 [SMILES: O=C(O)c1ccc(C(F)(F)F)cc1NCc1ccc(F)cc1] | MS (m/z) 314.0 (M + H)⁺ | (4- fluorophenyl) methanamine |

### Intermediate 353

### 2-((2-Methyl-3-(trifluoromethyl)phenyl)amino)-5-(trifluoromethyl)nicotinic Acid

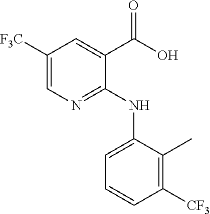 [SMILES: Cc1c(Nc2ncc(C(F)(F)F)cc2C(=O)O)cccc1C(F)(F)F]

To a solution containing 2-methyl-3-(trifluoromethyl)aniline (1.242 g, 7.09 mmol) in acetic acid (5 mL) was added 2-chloro-5-(trifluoromethyl)nicotinic acid (1 g, 4.43 mmol). The reaction was warmed to 100° C. for 18 hr. The reaction was cooled to RT and treated with 1N KOH and solid KOH to pH 12. The reaction was filtered and the filtrate was acidified with 1N HCl to pH 3. Filtered and washed with water. Solid was taken up in ethyl acetate, dried over MgSO₄, filtered and concentrated to afford title compound as a tan solid (590 mg, 1.620 mmol, 36.5% yield). MS (m/z) 365.1 (M+H)⁺.

Intermediates 354-356 were prepared from the indicated amine and carboxylic acid by methods analogous to those described for Intermediate 353.

| Int. | Name | Structure | Characterization | Amine | Acid |
|---|---|---|---|---|---|
| 354 | 2-((3-chloro-2- methylphenyl) amino)-5- (trifluoromethyl) nicotinic acid | 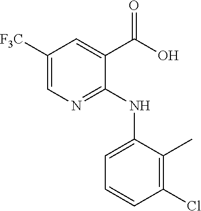 [SMILES: Cc1c(Cl)cccc1Nc1ncc(C(F)(F)F)cc1C(=O)O] | MS (m/z) 331.1 (M + H)⁺ | 3-chloro-2- methylaniline | 2-chloro-5- (trifluoromethyl) nicotinic acid |
| 355 | 2-((2-ethyl-4- fluorophenyl) amino)-5- (trifluoromethyl) nicotinic acid | 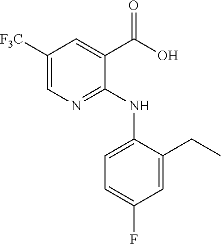 [SMILES: CCc1cc(F)ccc1Nc1ncc(C(F)(F)F)cc1C(=O)O] | MS (m/z) 329.2 (M + H)⁺ | 2-ethyl-4- fluoroaniline | 2-chloro-5- (trifluoromethyl) nicotinic acid |
| 356 | 2-((3,4-difluoro-2- methylphenyl) amino)-5- (trifluoromethyl) nicotinic acid | 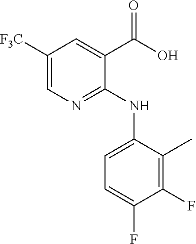 [SMILES: Cc1c(Nc2ncc(C(F)(F)F)cc2C(=O)O)ccc(F)c1F] | MS (m/z) 333.1 (M + H)⁺ | 3,4-difluoro-2- methylaniline | 2-chloro-5- (trifluoromethyl) nicotinic acid |

### Intermediate 357

### 6-Chloro-5-fluoro-2-((4-fluoro-2-methylphenyl)amino)nicotinic Acid

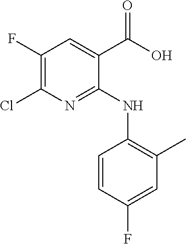 [SMILES: Cc1cc(F)ccc1Nc1nc(Cl)c(F)cc1C(=O)O]

To stirring solution of 2,6-dichloro-5-fluoronicotinic acid (250 mg, 1.191 mmol) and 4-fluoro-2-methylaniline (0.132 mL, 1.191 mmol) in THF (15 mL), at ambient temperature, was added LiHMDS (1.0 M in THF) (3.57 mL, 3.57 mmol) portion wise over one minute. Stirring was continued for a further 90 minutes. The reaction mixture was diluted with 2M HCl (aq) (2 mL) followed by concentration under a stream of nitrogen. The residue was dissolved in water (2 mL) and EtOAc (8 mL). The layers were separated, the pH of aqueous layer was adjusted to pH=2 with 2M HCl(aq) followed by re-extraction into EtOAc (˜8 mL). The combined organic extracts were dried by filtration through a hydrophobic frit and concentrated under a stream of nitrogen. The residue combined with the material that was obtained from a separate reaction carried out on 200 mg scale (acid). The material was dissolved in DMSO and purified by reverse phase column chromatography (Isco, 50 g RediSep C18 Gold column, water (0.1% formic acid):acetonitrile 10-100% over 40 minutes) to give the title compound (221 mg, 0.74 mmol, 35% yield). MS (m/z) 299 (M+H)⁺. 1 H NMR (DMSO-d₆, 600 MHz): δ (ppm) 14.03 (br s, 1H), 10.13 (br s, 1H), 8.21 (d, J=8.4 Hz, 1H), 7.91 (dd, J=8.9, 5.6 Hz, 1H), 7.14 (dd, J=9.5, 2.9 Hz, 1H), 7.04-7.09 (m, 1H), 2.26 (s, 3H).

### Intermediate 358

### 2-(Benzylamino)-5-(trifluoromethyl)benzoic Acid

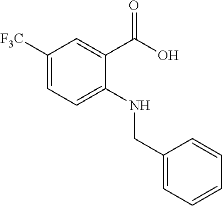 [SMILES: O=C(O)c1cc(C(F)(F)F)ccc1NCc1ccccc1]

To a stirring solution of 2-fluoro-5-(trifluoromethyl)benzoic acid (9.50 g, 45.6 mmol) in DMSO (100 mL) under nitrogen at 0° C. were added K₂CO₃ (25.2 g, 183 mmol) and benzylamine (9.97 mL, 91 mmol). The resulting reaction mixture was stirred at 100° C. for 24 hours. Upon completion, the reaction mixture was cooled to room temperature and ice cold water (100 mL) was added. The resulting mixture was acidified with 1.5N HCl till pH=4-5. Upon acidification formation of off-white solid was observed. The resulting suspension was stirred at room temperature for 1 hour. After 1 hour, the precipitated solid was filtered, washed with ice cold water (1000 mL) and dried under vacuum to obtain the title compound as an off-white solid (12.9 g, 40.8 mmol, 89% yield). MS (m/z) 296.0 (M+H)⁺.

### Intermediate 359

### 2-((4-Fluoro-2-methylphenyl)amino)-N-(6-methoxy-2-methylpyridin-3-yl)-4-(trifluoromethyl)benzamide

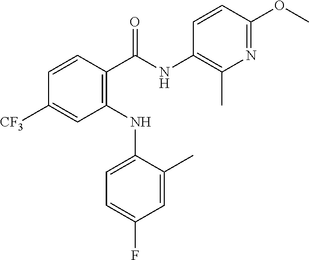 [SMILES: COc1ccc(NC(=O)c2ccc(C(F)(F)F)cc2Nc2ccc(F)cc2C)c(C)n1]

A solution of 6-methoxy-2-methylpyridin-3-amine (265 mg, 1.915 mmol) in DMF (0.5 mL) was added dropwise to a stirring solution of 2-((4-fluoro-2-methylphenyl)amino)-4-(trifluoromethyl)benzoic acid (500 mg, 1.596 mmol), HATU (910 mg, 2.394 mmol), and DIPEA (0.836 mL, 4.79 mmol) in DMF (3.0 mL) at 0° C. under N₂. After stirring at 28° C. for 16 hours, the reaction mixture was poured into ice-water (100 mL) and stirred at RT for 1 hour. The precipitate was filtered and vacuum dried for 2 hours to give the title compound as a brown solid (650 mg, 1.449 mmol, 91% yield). MS (m/z) 433.9 (M+H)⁺.

Intermediates 360-577 were prepared from the indicated amine and carboxylic acid by methods analogous to those described for Intermediate 359.

| Int. | Name | Structure | Characterization | Amine | Acid |
|---|---|---|---|---|---|
| 360 | 2-((4-fluoro-2- methylphenyl) amino)-N-(6- methoxypyridin- 3-yl)-4- (trifluoromethyl) benzamide | 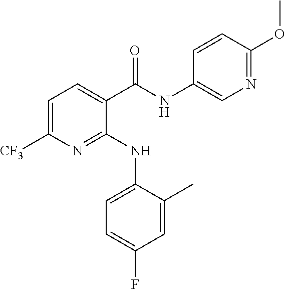 [SMILES: COc1ccc(NC(=O)c2ccc(C(F)(F)F)nc2Nc2ccc(F)cc2C)cn1] | MS (m/z) 420.0 (M + H)⁺ | 6- methoxypyridin- 3-amine | 2-((4-fluoro-2- methylphenyl) amino)-4- (trifluoromethyl) benzoic acid |
| 361 | 2- (cyclohexylamino)- N-(6- methoxypyridin- 3-yl)-4- (trifluoromethyl) benzamide | 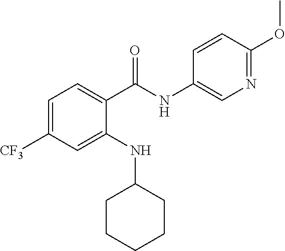 [SMILES: COc1ccc(NC(=O)c2ccc(C(F)(F)F)cc2NC2CCCCC2)cn1] | MS (m/z) 394.2 (M + H)⁺ | 6- methoxypyridin- 3-amine | 2- (cyclohexylamino)- 4-(trifluoromethyl) benzoic acid |
| 362 | 2-((4-fluoro-2,6- dimethylphenyl) amino)-N-(6- methoxypyridin- 3-yl)-4- (trifluoromethyl) benzamide | 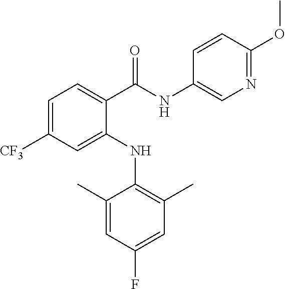 [SMILES: COc1ccc(NC(=O)c2ccc(C(F)(F)F)cc2Nc2c(C)cc(F)cc2C)cn1] | MS (m/z) 433.9 (M + H)⁺ | 6- methoxypyridin- 3-amine | 2-((4-fluoro-2,6- dimethylphenyl) amino)- 4-(trifluoromethyl) benzoic acid |
| 363 | 2-((2-chloro-4- fluorophenyl) amino)-N-(6- methoxypyridin- 3-yl)-4- (trifluoromethyl) benzamide | 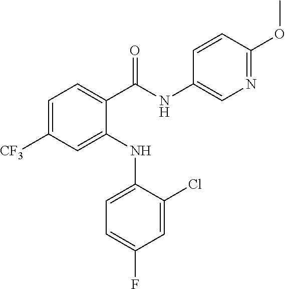 [SMILES: COc1ccc(NC(=O)c2ccc(C(F)(F)F)cc2Nc2ccc(F)cc2Cl)cn1] | MS (m/z) 440.0 (M + H)⁺ | 6- methoxypyridin- 3-amine | 2-((2-chloro-4- fluorophenyl) amino)- 4-(trifluoromethyl) benzoic acid |
| 364 | 2-((4-fluoro-2-(2- methoxyethoxy) phenyl)amino)- N-(6- methoxypyridin- 3-yl)-4- (trifluoromethyl) benzamide | 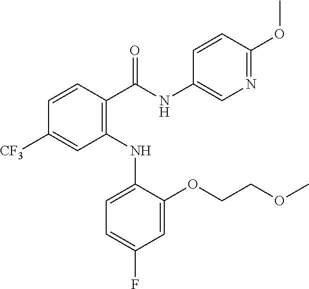 [SMILES: COCCOc1cc(F)ccc1Nc1cc(C(F)(F)F)ccc1C(=O)Nc1ccc(OC)nc1] | MS (m/z) 480.0 (M + H)⁺ | 6- methoxypyridin- 3-amine | 2-((4-fluoro-2-(2- methoxyethoxy) phenyl)amino)- 4-(trifluoromethyl) benzoic acid |
| 365 | 2-((2,4- difluorophenyl) amino)-N-(6- methoxypyridin- 3-yl)-4- (trifluoromethyl) benzamide | 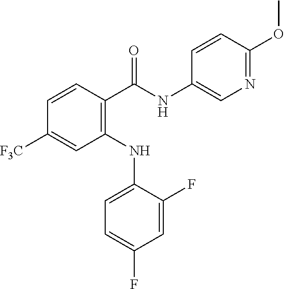 [SMILES: COc1ccc(NC(=O)c2ccc(C(F)(F)F)cc2Nc2ccc(F)cc2F)cn1] | MS (m/z) 423.9 (M + H)⁺ | 6- methoxypyridin- 3-amine | 2-((2,4- difluorophenyl) amino)- 4-(trifluoromethyl) benzoic acid |
| 366 | 2-((2-ethyl-4- fluorophenyl) amino)-N-(6- methoxypyridin- 3-yl)-4- (trifluoromethyl) benzamide | 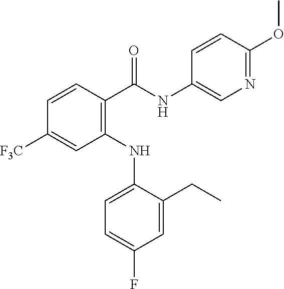 [SMILES: CCc1cc(F)ccc1Nc1cc(C(F)(F)F)ccc1C(=O)Nc1ccc(OC)nc1] | MS (m/z) 434.0 (M + H)⁺ | 6- methoxypyridin- 3-amine | 2-((2-ethyl-4- fluorophenyl) amino)- 4-(trifluoromethyl) benzoic acid |
| 367 | N-(6-methoxy-2- methylpyridin-3- yl)-2-((2- methylpyridin-3- yl)amino)-4- (trifluoromethyl) benzamide | 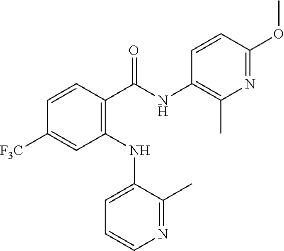 [SMILES: COc1ccc(NC(=O)c2ccc(C(F)(F)F)cc2Nc2cccnc2C)c(C)n1] | MS (m/z) 417.2 (M + H)⁺ | 6-methoxy-2- methylpyridin- 3-amine | 2-((2- methylpyridin-3- yl)-4- (trifluoromethyl) benzoic acid |
| 368 | 2-((4-fluoro-2- isopropyiphenyl) amino)-N-(6- methoxy-2- methylpyridin-3- yl)-4- (trifluoromethyl) benzamide | 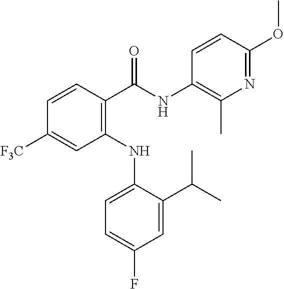 [SMILES: COc1ccc(NC(=O)c2ccc(C(F)(F)F)cc2Nc2ccc(F)cc2C(C)C)c(C)n1] | MS (m/z) 462.2 (M + H)⁺ | 6-methoxy-2- methylpyridin- 3-amine | 2-((4-fluoro-2- isopropyiphenyl) amino)- 4- (trifluoromethyl) benzoic acid |
| 369 | 5-chloro-2-((4- fluoro-2- methylphenyl) amino)-N-(6- methoxy-2- methylpyridin-3- yl)benzamide | 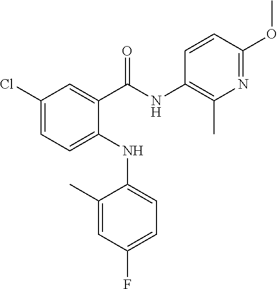 [SMILES: COc1ccc(NC(=O)c2cc(Cl)ccc2Nc2ccc(F)cc2C)c(C)n1] | MS (m/z) 400.0 (M + H)⁺ | 6-methoxy-2- methylpyridin- 3-amine | 5-chloro-2-((4- fluoro-2- methylphenyl) amino)benzoic acid |
| 370 | 2-((4-fluoro-2- methylphenyl) amino)-N-(6- methoxy-2- methylpyridin-3- yl)benzamide | 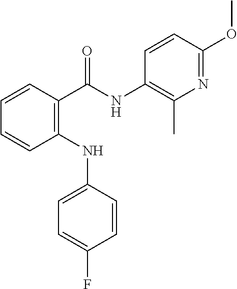 [SMILES: COc1ccc(NC(=O)c2ccccc2Nc2ccc(F)cc2)c(C)n1] | MS (m/z) 352.0 (M + H)⁺ | 6-methoxy-2- methylpyridin- 3-amine | 2-((4- fluorophenyl)amino) benzoic acid |
| 371 | N-(6-methoxy-2- methylpyridin-3- yl)-2-(o-tolylamino) benzamide | 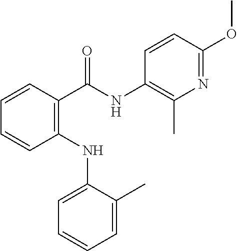 [SMILES: COc1ccc(NC(=O)c2ccccc2Nc2ccccc2C)c(C)n1] | MS (m/z) 348.2 (M + H)⁺ | 6-methoxy-2- methylpyridin- 3-amine | 2-(o-tolylamino) benzoic acid |
| 372 | 2-((4-fluoro-2- methylphenyl) amino)-N-(6- methoxy-2- methylpyridin-3- yl)-5- methylbenzamide | 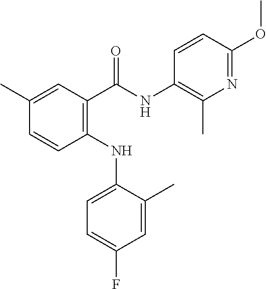 | MS (m/z) 380.2 (M + H)⁺ | 6-methoxy-2- methylpyridin- 3-amine | 2-((4-fluoro-2- methylphenyl) amino)-5- methylbenzoic acid |
| 373 | 2-((4-fluoro-2- methylphenyl) amino)-N-(6- methoxy-2- methylpyridin-3- yl)benzamide | 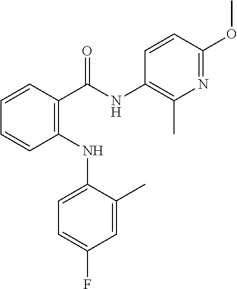 [SMILES: COc1ccc(NC(=O)c2ccccc2Nc2ccc(F)cc2C)c(C)n1] | MS (m/z) 366.1 (M + H)⁺ | 6-methoxy-2- methylpyridin- 3-amine | 2-((4-fluoro-2- methylphenyl) amino)benzoic acid |
| 374 | 2-((4-fluoro-2- methylphenyl) amino)-N-(2- methoxypyrimidin- 5-yl)-4- (trifluoromethyl) benzamide | 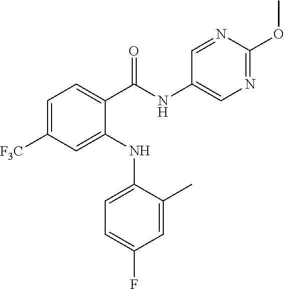 [SMILES: COc1ncc(NC(=O)c2ccc(C(F)(F)F)cc2Nc2ccc(F)cc2C)cn1] | MS (m/z) 421.3 (M + H)⁺ | 2- methoxypyrimidin- 5-amine | 2-((4-fluoro-2- methylphenyl) amino)-4- (trifluoromethyl) benzoic acid |
| 375 | 2-((4-bromo-2- methylphenyl) amino)-N-(6- methoxypyridin- 3-yl)-4- (trifluoromethyl) benzamide | 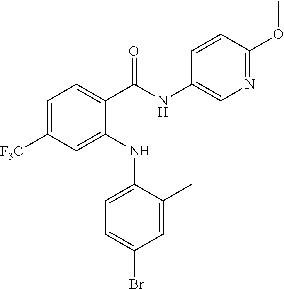 [SMILES: COc1ccc(NC(=O)c2ccc(C(F)(F)F)cc2Nc2ccc(Br)cc2C)cn1] | MS (m/z) 482.1 (M + H)⁺ | 6- methoxypyridin- 3-amine | 2-((4-bromo-2- methylphenyl) amino)-4- (trifluoromethyl) benzoic acid |
| 376 | 2-((4- fluorophenyl) amino)-N-(6- methoxy-2- methylpyridin-3- yl)-4- (trifluoromethyl) benzamide | 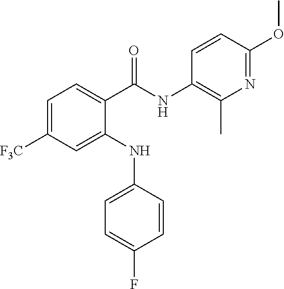 [SMILES: COc1ccc(NC(=O)c2ccc(C(F)(F)F)cc2Nc2ccc(F)cc2)c(C)n1] | MS (m/z) 420.3 (M + H)⁺ | 6-methoxy-2- methylpyridin- 3-amine | 2-((4- fluorophenyl) amino)-4- (trifluoromethyl) benzoic acid |
| 377 | 2-((4-fluoro-2- methylphenyl) amino)-N-(6- methoxy-2- methylpyridin-3- yl)-6- (trifluoromethyl) nicotinamide | 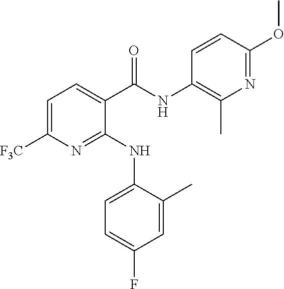 [SMILES: COc1ccc(NC(=O)c2ccc(C(F)(F)F)nc2Nc2ccc(F)cc2C)c(C)n1] | MS (m/z) 435.3 (M + H)⁺ | 6-methoxy-2- methylpyridin- 3-amine | 2-((4-fluoro-2- methylphenyl) amino)-4- (trifluoromethyl) benzoic acid |
| 378 | N-(2-ethyl-6- methoxypyridin- 3-yl)-2-((4-fluoro- 2- methylphenyl) amino)-4- (trifluoromethyl) benzamide | 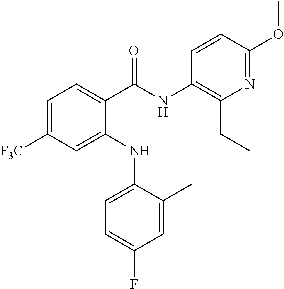 [SMILES: CCc1nc(OC)ccc1NC(=O)c1ccc(C(F)(F)F)cc1Nc1ccc(F)cc1C] | MS (m/z) 448.3 (M + H)⁺ | 2-ethyl- 6- methoxypyridin- 3-amine | 2-((4-fluoro-2- methylphenyl) amino)-4- (trifluoromethyl) benzoic acid |
| 379 | N-(2-chloro-6- methoxypyridin- 3-yl)-2-((4-fluoro- 2- methylphenyl) amino)-4- (trifluoromethyl) benzamide | 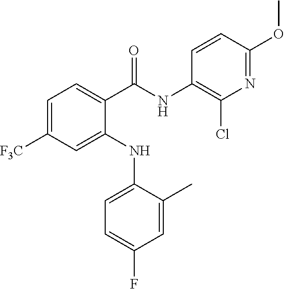 [SMILES: COc1ccc(NC(=O)c2ccc(C(F)(F)F)cc2Nc2ccc(F)cc2C)c(Cl)n1] | MS (m/z) 454.2 (M + H)⁺ | 2-chloro- 6- methoxypyridin- 3-amine, Hydrochloride | 2-((4-fluoro-2- methylphenyl) amino)-4- (trifluoromethyl) benzoic acid |
| 380 | 4-bromo-2-((4- fluoro-2- methylphenyl) amino)-N-(6- methoxy-2- methylpyridin-3- yl)benzamide | 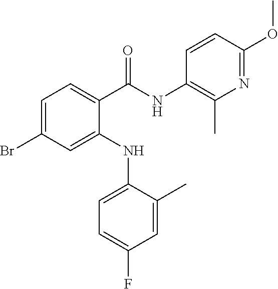 [SMILES: COc1ccc(NC(=O)c2ccc(Br)cc2Nc2ccc(F)cc2C)c(C)n1] | MS (m/z) 446.2 (M + H)⁺ | 6-methoxy-2- methylpyridin- 3-amine | 4-bromo- 2-((4-fluoro-2- methylphenyl) amino) benzoic acid |
| 381 | 2-((2,4- difluorophenyl) amino)-N-(6- methoxy-2- methylpyridin-3- yl)-4- (trifluoromethyl) benzamide | 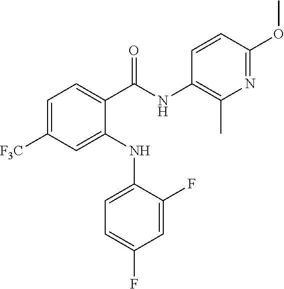 [SMILES: COc1ccc(NC(=O)c2ccc(C(F)(F)F)cc2Nc2ccc(F)cc2F)c(C)n1] | MS (m/z) 438.3 (M + H)⁺ | 6-methoxy-2- methylpyridin- 3-amine | 2-((2,4- difluorophenyl) amino)-4- (trifluoromethyl) benzoic acid |
| 382 | 2-((4- ethoxyphenyl) amino)-N-(6- methoxy-2- methylpyridin-3- yl)-4- (trifluoromethyl) benzamide | 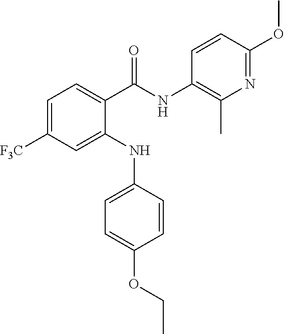 [SMILES: CCOc1ccc(Nc2cc(C(F)(F)F)ccc2C(=O)Nc2ccc(OC)nc2C)cc1] | MS (m/z) 446.3 (M + H)⁺ | 6-methoxy-2- methylpyridin- 3-amine | 2-((4- ethoxyphenyl) amino)-4- (trifluoromethyl) benzoic acid |
| 383 | 2-((4-fluoro-2- methylphenyl) amino)-N-(6- methoxypyridin- 3-yl)-5- (trifluoromethyl) benzamide | 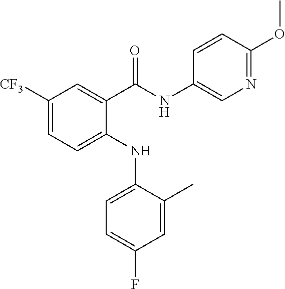 [SMILES: COc1ccc(NC(=O)c2cc(C(F)(F)F)ccc2Nc2ccc(F)cc2C)cn1] | MS (m/z) 420.0 (M + H)⁺ | 6- methoxypyridin- 3-amine | 2-((4-fluoro-2- methylphenyl) amino)-5- (trifluoromethyl) benzoic acid |
| 384 | 2-((4-fluoro-2- methoxyphenyl) amino)-N-(6- methoxypyridin- 3-yl)-4- (trifluoromethyl) benzamide | 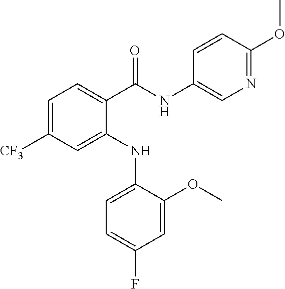 [SMILES: COc1ccc(NC(=O)c2ccc(C(F)(F)F)cc2Nc2ccc(F)cc2OC)cn1] | MS (m/z) 436.0 (M + H)⁺ | 6- methoxypyridin- 3-amine | 2-((4-fluoro-2- methylphenyl) amino)-4- (trifluoromethyl) benzoic acid |
| 385 | 2-((4-fluoro-2- methylphenyl) amino)-N-(6- methoxypyridin- 3-yl)-6- (trifluoromethyl) benzamide | 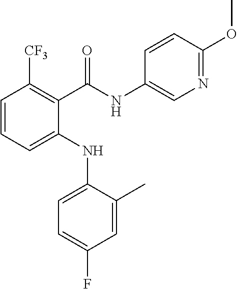 [SMILES: COc1ccc(NC(=O)c2c(Nc3ccc(F)cc3C)cccc2C(F)(F)F)cn1] | MS (m/z) 419.9 (M + H)⁺ | 6- methoxypyridin- 3-amine | 2-((4-fluoro-2- methylphenyl) amino)-6- (trifluoromethyl) benzoic acid |
| 386 | 2-((4-fluoro-2- methylphenyl) amino)-N-(6- methoxy-5- methylpyridin-3- yl)-4- (trifluoromethyl) benzamide | 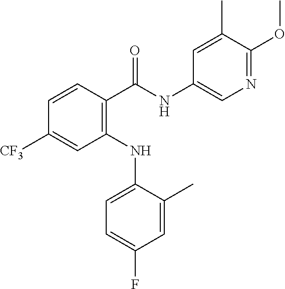 [SMILES: COc1ncc(NC(=O)c2ccc(C(F)(F)F)cc2Nc2ccc(F)cc2C)cc1C] | MS (m/z) 433.9 (M + H)⁺ | 6-methoxy-5- methylpyridin- 3-amine | 2-((4-fluoro-2- methylphenyl) amino)-4- (trifluoromethyl) benzoic acid |
| 387 | 2-((4-fluoro-2- methylphenyl) amino)-N-(6- methoxypyridazin- 3-yl)-4- (trifluoromethyl) benzamide | 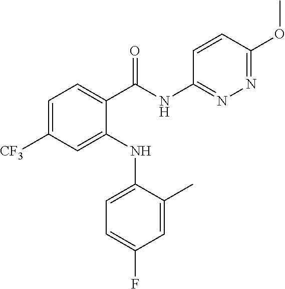 [SMILES: COc1ccc(NC(=O)c2ccc(C(F)(F)F)cc2Nc2ccc(F)cc2C)nn1] | MS (m/z) 421.0 (M + H)⁺ | 6- methoxypyridin- 3-amine | 2-((4-fluoro-2- methylphenyl) amino)-4- (trifluoromethyl) benzoic acid |
| 388 | 2-((4-fluoro-2- methylphenyl) amino)-N-(6- methoxy-4- methylpyridin-3- yl)-4- (trifluoromethyl) benzamide | 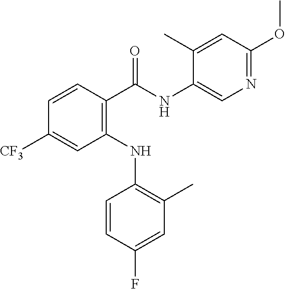 [SMILES: COc1cc(C)c(NC(=O)c2ccc(C(F)(F)F)cc2Nc2ccc(F)cc2C)cn1] | MS (m/z) 433.9 (M + H)⁺ | 6-methoxy-4- methylpyridin- 3-amine | 2-((4-fluoro-2- methylphenyl) amino)-4- (trifluoromethyl) benzoic acid |
| 389 | 2-((4-fluoro-2- methoxyphenyl) amino)-N-(6- methoxypyridin- 3-yl)-6- (trifluoromethyl) benzamide | 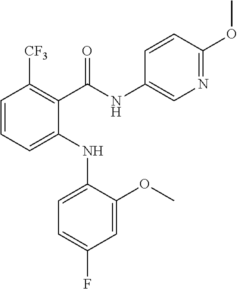 [SMILES: COc1ccc(NC(=O)c2c(Nc3ccc(F)cc3OC)cccc2C(F)(F)F)cn1] | MS (m/z) 436.0 (M + H)⁺ | 6- methoxypyridin- 3-amine | 2-((4-fluoro-2- methylphenyl) amino)-6- (trifluoromethyl) benzoic acid |
| 390 | 2-((4-bromo-2- methylphenyl) amino)-N-(6- methoxy-2- methylpyridin-3- yl)-4- (trifluoromethyl) benzamide | 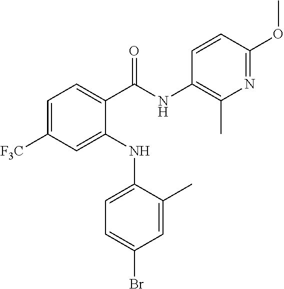 [SMILES: COc1ccc(NC(=O)c2ccc(C(F)(F)F)cc2Nc2ccc(Br)cc2C)c(C)n1] | MS (m/z) 496.1 (M + H)⁺ | 6-methoxy-2- methylpyridin- 3-amine | 2-((4-bromo-2- methylphenyl) amino)-4- (trifluoromethyl) benzoic acid |
| 391 | N-(2-bromo-6- methoxypyridin- 3-yl)-2-((4-fluoro- 2- methylphenyl) amino)-4- (trifluoromethyl) benzamide | 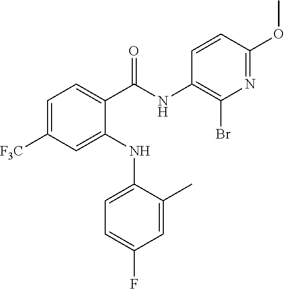 [SMILES: COc1ccc(NC(=O)c2ccc(C(F)(F)F)cc2Nc2ccc(F)cc2C)c(Br)n1] | MS (m/z) 499.9 (M + H)⁺ | 2-bromo-6- methylpyridin- 3-amine | 2-((4-fluoro-2- methylphenyl) amino)-4- (trifluoromethyl) benzoic acid |
| 392 | 2-((4- fluorophenyl) amino)-N-(6- methoxypyridin- 3-yl)-4- (trifluoromethyl) benzamide | 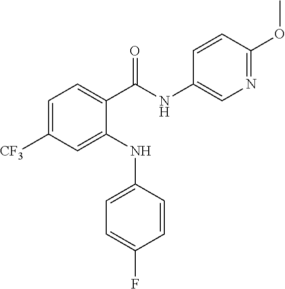 [SMILES: COc1ccc(NC(=O)c2ccc(C(F)(F)F)cc2Nc2ccc(F)cc2)cn1] | MS (m/z) 406.3 (M + H)⁺ | 6- methoxypyridin- 3-amine | 2-((4- fluorophenyl) amino)-4- (trifluoromethyl) benzoic acid |
| 393 | 2-((4- fluorobenzyl) amino)-N-(6- methoxypyridin- 3-yl)-4- (trifluoromethyl) benzamide | 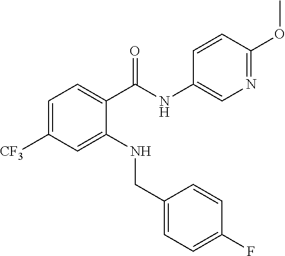 [SMILES: COc1ccc(NC(=O)c2ccc(C(F)(F)F)cc2NCc2ccc(F)cc2)cn1] | MS (m/z) 419.9 (M + H)⁺ | 6- methoxypyridin- 3-amine | 2-((4- fluorobenzyl) amino)-4- (trifluoromethyl) benzoic acid |
| 394 | 4-cyano-2- fluoro-N-(6- methoxypyridin- 3-yl)benzamide | 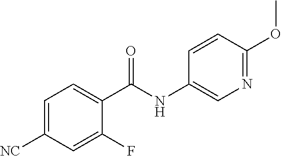 [SMILES: COc1ccc(NC(=O)c2ccc(C#N)cc2F)cn1] | MS (m/z) 271.9 (M + H)⁺ | 6- methoxypyridin- 3-amine | 4-cyano-2- fluorobenzoic acid |
| 395 | 3-chloro-2- fluoro-N-(6- methoxypyridin- 3-yl)-4- (trifluoromethyl) benzamide | 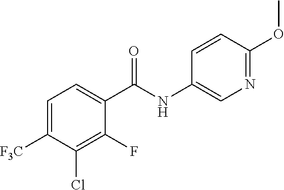 [SMILES: COc1ccc(NC(=O)c2ccc(C(F)(F)F)c(Cl)c2F)cn1] | MS (m/z) 348.9 (M + H)⁺ | 6- methoxypyridin- 3-amine | 3-chloro-2- fluoro-4- (trifluoromethyl) benzoic acid |
| 396 | 2-((4-fluoro-2- methylphenyl) amino)-N-(2- methoxypyridin- 4-yl)-4- (trifluoromethyl) benzamide | 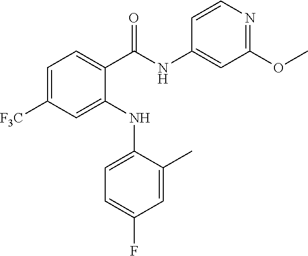 [SMILES: COc1cc(NC(=O)c2ccc(C(F)(F)F)cc2Nc2ccc(F)cc2C)ccn1] | MS (m/z) 420.3 (M + H)⁺ | 2- methoxypyridin- 4-amine | 2-((4-fluoro-2- methylphenyl) amino)-4- (trifluoromethyl) benzoic acid |
| 397 | 2-((4-fluoro-2- methylphenyl) amino)-N-(2- methoxy-6- methylpyridin-4- yl)-4- (trifluoromethyl) benzamide | 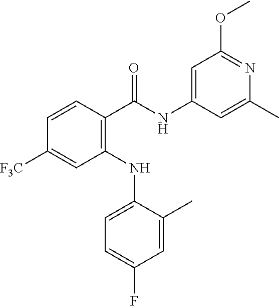 [SMILES: COc1cc(NC(=O)c2ccc(C(F)(F)F)cc2Nc2ccc(F)cc2C)cc(C)n1] | MS (m/z) 434.0 (M + H)⁺ | 2-methoxy-6- methylpyridin- 4-amine | 2-((4-fluoro-2- methylphenyl) amino)-4- (trifluoromethyl) benzoic acid |
| 398 | 2-((4-fluoro-2- methylphenyl) amino)-N-(2- methoxy-5- methylpyridin-4- yl)-4- (trifluoromethyl) benzamide | 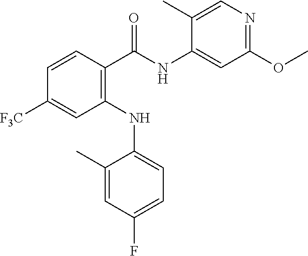 [SMILES: COc1cc(NC(=O)c2ccc(C(F)(F)F)cc2Nc2ccc(F)cc2C)c(C)cn1] | MS (m/z) 434.0 (M + H)⁺ | 2-methoxy-5- methylpyridin- 4-amine | 2-((4-fluoro-2- methylphenyl) amino)-4- (trifluoromethyl) benzoic acid |
| 399 | 2-((4-fluoro-2- methylphenyl) amino)-N-(2- methoxy-3- methylpyridin-4- yl)-4- (trifluoromethyl) benzamide | 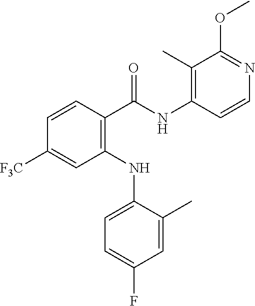 [SMILES: COc1nccc(NC(=O)c2ccc(C(F)(F)F)cc2Nc2ccc(F)cc2C)c1C] | MS (m/z) 434.0 (M + H)⁺ | 2-methoxy-3- methylpyridin- 4-amine | 2-((4-fluoro-2- methylphenyl) amino)-4- (trifluoromethyl) benzoic acid |
| 400 | 2-((4-fluoro-2- methylphenyl) amino)-N-(1- methyl-6-oxo- 1,6- dihydropyridin-3- yl)-4- (trifluoromethyl) benzamide | 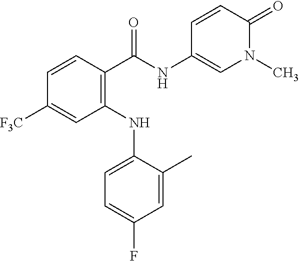 [SMILES: Cc1cc(F)ccc1Nc1cc(C(F)(F)F)ccc1C(=O)Nc1ccc(=O)n(C)c1] | MS (m/z) 420.0 (M + H)⁺ | 5-amino-1- methylpyridin- 2(1H)-one | 2-((4-fluoro-2- methylphenyl) amino)-4- (trifluoromethyl) benzoic acid |
| 401 | 2-((4-fluoro-2- methylphenyl) amino)-N-(6- methoxy-2- methylpyridin-3- yl)-4- (trifluoromethoxy) benzamide | 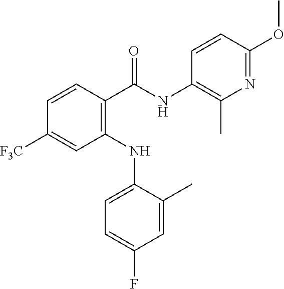 | MS (m/z) 450.1 (M + H)⁺ | 6-methoxy-2- methylpyridin- 3-amine | 2-((4-fluoro-2- methylphenyl) amino)-4- (trifluoromethoxy) benzoic acid |
| 402 | 4-chloro-2-((4- fluoro-2- methylphenyl) amino)-N-(6- methoxy-2- methylpyridin-3- yl)benzamide | 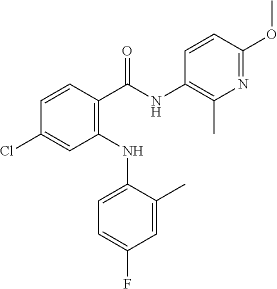 [SMILES: COc1ccc(NC(=O)c2ccc(Cl)cc2Nc2ccc(F)cc2C)c(C)n1] | MS (m/z) 400.0 (M + H)⁺ | 6-methoxy-2- methylpyridin- 3-amine | 4-chloro-2-((4- fluoro-2- methylphenyl) amino)benzoic acid |
| 403 | 2-((4-fluoro-2- methylphenyl) amino)-N-(6- methoxy-2- methylpyridin-3- yl)-5- (trifluoromethyl) benzamide | 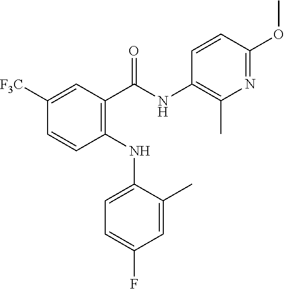 [SMILES: COc1ccc(NC(=O)c2cc(C(F)(F)F)ccc2Nc2ccc(F)cc2C)c(C)n1] | MS (m/z) 434.0 (M + H)⁺ | 6-methoxy-2- methylpyridin- 3-amine | 2-((4-fluoro-2- methylphenyl) amino)-5- (trifluoromethyl) benzoic acid |

### Intermediate 403

### 2-((4-fluoro-2-methylphenyl)amino)-N-(6-methoxy-2-methylpyridin-3-yl)-5-(trifluoromethyl)benzamide

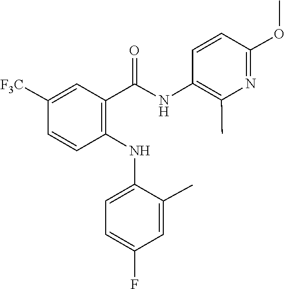 [SMILES: COc1ccc(NC(=O)c2cc(C(F)(F)F)ccc2Nc2ccc(F)cc2C)c(C)n1]

To a solution of 2-((4-fluoro-2-methylphenyl)amino)-5-(trifluoromethyl)benzoic acid (2.1 g, 6.7 mmol), DIPEA (2.34 mL, 13.4 mmol) and HATU (3.82 g, 10.1 mmol) in DMF (20 mL) under nitrogen at RT was added 6-methoxy-2-methylpyridin-3-amine (1.02 g, 7.4 mmol) dropwise over 1 min. The reaction mixture was stirred at RT for 12 h. The reaction mixture was quenched with ice cold water (100 mL) and resulting solid was filtered and dried under vacuum to afford the title compound as a brown solid (2.4 g, 5.5 mmol, 82% yield). MS (m/z) 434.0 (M+H)⁺.

| | | | | | |
|---|---|---|---|---|---|
| 404 | 2-((4-fluoro-2- methylphenyl)- amino)-N-(5- methoxy-3- methylpyrazin-2- yl)-4- (trifluoromethyl)- benzamide | 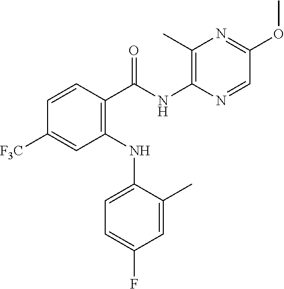 [SMILES: COc1cnc(NC(=O)c2ccc(C(F)(F)F)cc2Nc2ccc(F)cc2C)c(C)n1] | MS (m/z) 435.0 (M + H)⁺ | 5- meth- oxy-3- methyl pyrazin- 2- amine | 2-((4-fluoro-2- methylphenyl) amino)-4- (trifluoromethyl)- benzoic acid |
| 405 | 4,5-difluoro-2- ((4-fluoro-2- methylphenyl)- amino)-N-(6- methoxy-2- methylpyridin-3- yl)benzamide | 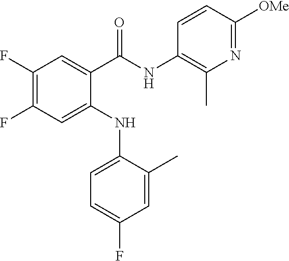 [SMILES: COc1ccc(NC(=O)c2cc(F)c(F)cc2Nc2ccc(F)cc2C)c(C)n1] | MS (m/z) 402.3 (M + H)⁺ | 6- meth- oxy-2- methyl pyridin- 3- amine | 4,5-difluoro-2- ((4-fluoro-2- methylphenyl) amino)benzoic acid |
| 406 | N-(6-methoxy-2- methylpyridin-3- yl)-2-((2- methylcyclohexyl)- amino)-4- (trifluoromethyl)- benzamide | 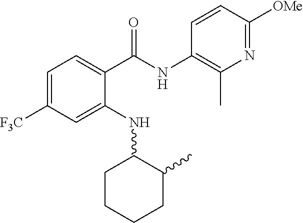 [SMILES: COc1ccc(NC(=O)c2ccc(C(F)(F)F)cc2NC2CCCCC2C)c(C)n1] | MS (m/z) 422.3 (M + H)⁺ | 6- meth- oxy-2- methyl pyridin- 3- amine | 2-((2- methylcyclo- hexyl)amino)-4- (trifluoromethyl)- benzoic acid |
| 407 | 4,5-difluoro-2- ((4-fluoro-2- isopropylphenyl) amino)-N-(6- methoxy-2- methylpyridin-3- yl)benzamide | 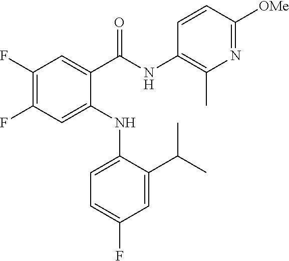 [SMILES: COc1ccc(NC(=O)c2cc(F)c(F)cc2Nc2ccc(F)cc2C(C)C)c(C)n1] | MS (m/z) 430.3 (M + H)⁺ | 6- meth- oxy-2- methyl pyridin- 3- amine | 4,5-difluoro-2- ((4-fluoro-2- isopropylphen- yl)amino)benzoic acid |
| 408 | N-(6-methoxy-2- methylpyridin-3- yl)-2-(((1S,2R)- 2- methylcyclohexyl)- amino)-4- (trifluoromethyl)- benzamide | 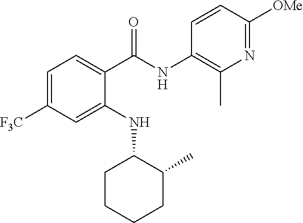 [SMILES: COc1ccc(NC(=O)c2ccc(C(F)(F)F)cc2N[C@H]2CCCC[C@H]2C)c(C)n1] | MS (m/z) 422.3 (M + H)⁺ | 6- meth- oxy-2- methyl pyridin- 3- amine | 2-(((1S,2R)-2- methylcyclo- hexyl)amino)-4- (trifluoromethyl)- benzoic acid |
| 409 | N-(6-methoxy-2- methylpyridin-3- yl)-2-(((1R,2S)- 2- methylcyclohexyl)- amino)-4- (trifluoromethyl)- benzamide | 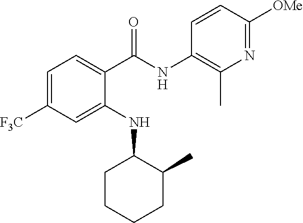 [SMILES: COc1ccc(NC(=O)c2ccc(C(F)(F)F)cc2N[C@@H]2CCCC[C@@H]2C)c(C)n1] | MS (m/z) 422.4 (M + H)⁺ | 6- meth- oxy-2- methyl pyridin- 3- amine | 2-(((1R,2S)-2- methylcyclo- hexyl)amino)-4- (trifluoromethyl)- benzoic acid |
| 410 | 4-cyano-2-((4- fluoro-2- isopropylphenyl) amino)-N-(6- methoxy-2- methylpyridin-3- yl)benzamide | 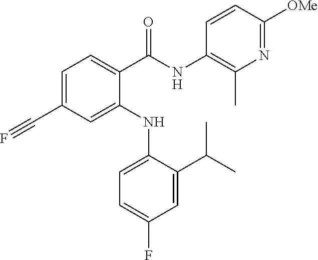 | MS (m/z) 419.3 (M + H)⁺ | 6- meth- oxy-2- methyl pyridin- 3- amine | 4-cyano-2-((4- fluoro-2- isopropylphen- yl)amino)benzoic acid |
| 411 | 2-((2-ethyl-4- fluorophenyl)- amino)-N-(6- methoxy-2- methylpyridin-3- yl)-5- (trifluoromethyl)- benzamide | 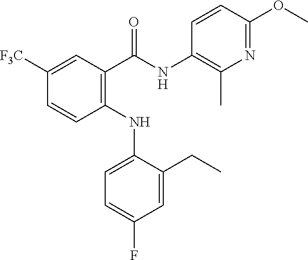 [SMILES: CCc1cc(F)ccc1Nc1ccc(C(F)(F)F)cc1C(=O)Nc1ccc(OC)nc1C] | MS (m/z) 448.0 (M + H)⁺ | 6- meth- oxy-2- methyl pyridin- 3- amine | 2-((2-ethyl-4- fluorophenyl)- amino)-5- (trifluoromethyl)- benzoic acid |
| 412 | N-(6-methoxy-2- methylpyridin-3- yl)-2-((4- methylthiazol-5- yl)amino)-5- (trifluoromethyl)- benzamide | 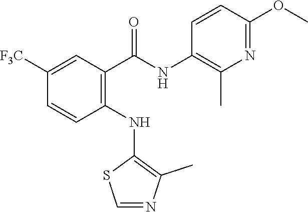 [SMILES: COc1ccc(NC(=O)c2cc(C(F)(F)F)ccc2Nc2scnc2C)c(C)n1] | MS (m/z) 423.0 (M + H)⁺ | 6- meth- oxy-2- methyl pyridin- 3- amine | 2-((4- methylthiazol- 5-yl)amino)-5- (trifluoromethyl)- benzoic acid |
| 413 | 5-chloro-2-((4- fluoro-2- methylphenyl)- amino)-N-(2- methoxy-4- methylpyrimidin- 5-yl)benzamide | 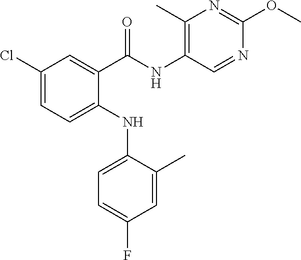 [SMILES: COc1ncc(NC(=O)c2cc(Cl)ccc2Nc2ccc(F)cc2C)c(C)n1] | MS (m/z) 401.0 (M + H)⁺ | 2- meth- oxy-4- methyl pyrimidin- 5- amine | 5-chloro-2-((4- fluoro-2- methylphenyl) amino)benzoic acid |
| 414 | 5-fluoro-2-((4- fluoro-2- methylphenyl)- amino)-6- methoxy-N-(6- methoxy-2- methylpyridin-3- yl)nicotinamide | 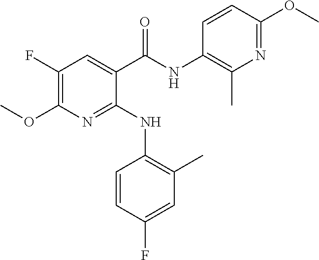 | MS (m/z) 415.0 (M + H)⁺ | 6- meth- oxy-2- methyl pyridin- 3- amine | 5-fluoro-2-((4- fluoro-2- methylphenyl) amino)-6- methoxynicotinic acid |
| 415 | 5-cyano-2-((4- fluoro-2- methylphenyl)- amino)-N-(6- methoxy-2- methylpyridin-3- yl)-4- (trifluoromethyl)- benzamide | 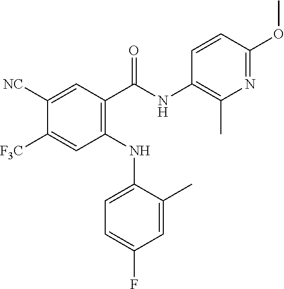 [SMILES: COc1ccc(NC(=O)c2cc(C#N)c(C(F)(F)F)cc2Nc2ccc(F)cc2C)c(C)n1] | MS (m/z) 459.0 (M + H)⁺ | 6- meth- oxy-2- methyl pyridin- 3- amine | 5-cyano-2-((4- fluoro-2- methylphenyl) amino)-4- (trifluoromethyl)- benzoic acid |
| 416 | 5-chloro-4- (difluoromethoxy) -2-((4-fluoro-2- methylphenyl)- amino)-N-(6- methoxy-2- methylpyridin-3- yl)benzamide | 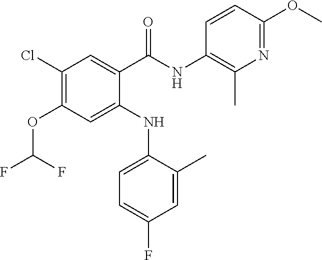 [SMILES: COc1ccc(NC(=O)c2cc(Cl)c(OC(F)F)cc2Nc2ccc(F)cc2C)c(C)n1] | MS (m/z) 466.3 (M + H)⁺ | 6- meth- oxy-2- methyl pyridin- 3- amine | 5-chloro-4- (difluoro- methoxy)- 2-((4- fluoro-2- methylphenyl) amino)benzoic acid |
| 417 | 5-chloro-4- (difluoromethyl)- 2-((4-fluoro-2- methylphenyl)- amino)-N-(6- methoxy-2- methylpyridin-3- yl)benzamide | 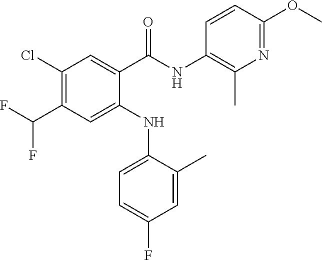 [SMILES: COc1ccc(NC(=O)c2cc(Cl)c(C(F)F)cc2Nc2ccc(F)cc2C)c(C)n1] | MS (m/z) 450.3/ 452.3 (M + H)⁺ | 6- meth- oxy-2- methyl pyridin- 3- amine | 5-chloro-4- (difluoromethyl)- 2-((4-fluoro- 2- methylphenyl) amino)benzoic acid |
| 418 | 5-chloro-4- (difluoromethyl)- 2-((4-fluoro-2- methylphenyl)- amino)-N-(3- methylpyridin-4- yl)benzamide | 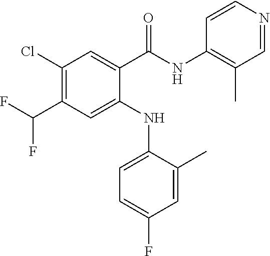 [SMILES: Cc1cnccc1NC(=O)c1cc(Cl)c(C(F)F)cc1Nc1ccc(F)cc1C] | MS (m/z) 420.3 (M + H)^{+/ 422.3} ^{(M+3H)+} | 3- methyl pyridin- 4- amine | 5-chloro-4- (difluoromethyl)- 2-((4-fluoro- 2- methylphenyl) amino)benzoic acid |
| 419 | N-(6-bromo-2- methylpyridin-3- yl)-2-((4-fluoro-2- methylphenyl)- amino)-5- (trifluoromethyl)- benzamide | 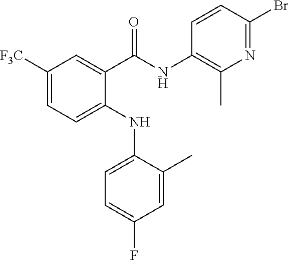 [SMILES: Cc1cc(F)ccc1Nc1ccc(C(F)(F)F)cc1C(=O)Nc1ccc(Br)nc1C] | MS (m/z) 482.2 (M + H)7 484.2 (M+3H)⁺ | 6- bromo- 2- methyl pyridin- 3- amine | 2-((4-fluoro-2- methylphenyl) amino)-5- (trifluoromethyl)- benzoic acid |
| 420 | 5-chloro-6- cyano-N-(6- methoxy-2- methylpyridin-3- yl)-2-((2-methyl- 4- (trifluoromethoxy)- phenyl)amino)- nicotinamide | 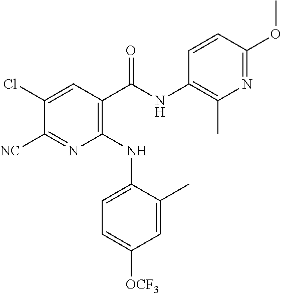 [SMILES: COc1ccc(NC(=O)c2cc(Cl)c(C#N)nc2Nc2ccc(OC(F)(F)F)cc2C)c(C)n1] | MS (m/z) 492.3 (M + H)⁺ | 6- meth- oxy-2- methyl pyridin- 3- amine | 5-chloro-6- cyano-2-((2- methyl-4- (trifluorometh- oxy)phenyl)- amino)nicotinic acid |
| 421 | 2-((2,4- dimethoxyphenyl)- amino)-N-(6- methoxy-2- methylpyridin-3- yl)-5- (trifluoromethyl)- benzamide | 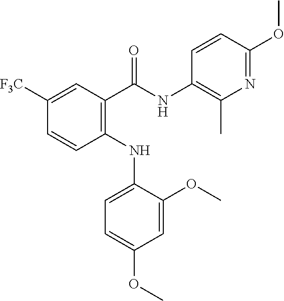 [SMILES: COc1ccc(Nc2ccc(C(F)(F)F)cc2C(=O)Nc2ccc(OC)nc2C)c(OC)c1] | MS (m/z) 462.3 (M + H)⁺ | 6- meth- oxy-2- methyl pyridin- 3- amine | 2-((2,4- dimethoxyphe nyl)amino)-5- (trifluoromethyl)- benzoic acid |
| 422 | 2-((4-fluoro-2- methoxyphenyl) amino)-N-(6- methoxy-2- methylpyridin-3- yl)-5- (trifluoromethyl)- benzamide | 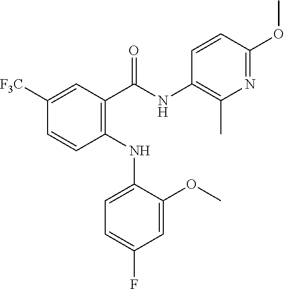 [SMILES: COc1ccc(NC(=O)c2cc(C(F)(F)F)ccc2Nc2ccc(F)cc2OC)c(C)n1] | MS (m/z) 450.3 (M + H)⁺ | 6- meth- oxy-2- methyl pyridin- 3- amine | 2-((4-fluoro-2- methoxyphen- yl)amino)-5- (trifluoromethyl)- benzoic acid |
| 423 | 5-chloro-6- cyano-2-((4- fluoro-2- methylphenyl)- amino)-N-(6- methoxy-2- methylpyridin-3- yl)nicotinamide | 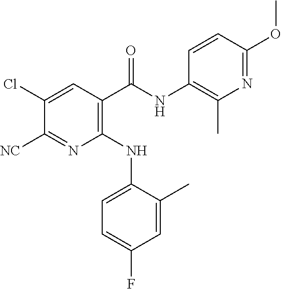 [SMILES: COc1ccc(NC(=O)c2cc(Cl)c(C#N)nc2Nc2ccc(F)cc2C)c(C)n1] | MS (m/z) 426.2 (M + H)⁺ | 6- meth- oxy-2- methyl pyridin- 3- amine | 5-chloro-6- cyano-2-((4- fluoro-2- methylphenyl) amino)nicotinic acid |
| 424 | 5-chloro-4- fluoro-N-(6- methoxy-2- methylpyridin-3- yl)-2-((2-methyl- 4- (trifluoromethoxy)- phenyl)amino)- benzamide | 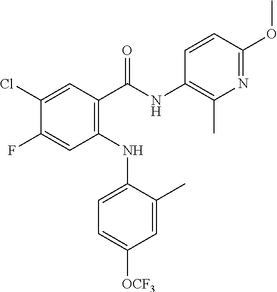 [SMILES: COc1ccc(NC(=O)c2cc(Cl)c(F)cc2Nc2ccc(OC(F)(F)F)cc2C)c(C)n1] | MS (m/z) 484.3 (M + H)⁺ | 6- meth- oxy-2- methyl pyridin- 3- amine | 5-chloro-4- fluoro-2-((2- methyl-4- (trifluorometh- oxy)phenyl)- amino)benzoic acid |
| 425 | 5-chloro-4- fluoro-2-((4- fluoro-2- methylphenyl)- amino)-N-(2- methoxy-4- methylpyrimidin- 5-yl)benzamide | 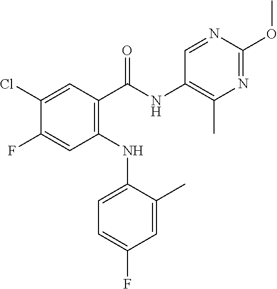 [SMILES: COc1ncc(NC(=O)c2cc(Cl)c(F)cc2Nc2ccc(F)cc2C)c(C)n1] | MS (m/z) 419.1 (M + H)⁺ | 2- meth- oxy-4- methyl pyrimidin- 5- amine | 5-chloro-4- fluoro-2-((4- fluoro-2- methylphenyl) amino)benzoic acid |
| 426 | N-(6-methoxy-2- methylpyridin-3- yl)-2-((2-methyl- 3- (trifluoromethyl)- phenyl)amino)-5- (trifluoromethyl)- nicotinamide | 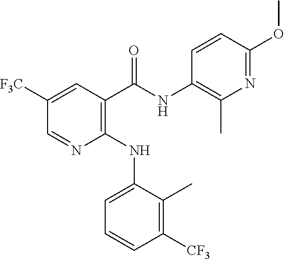 [SMILES: COc1ccc(NC(=O)c2cc(C(F)(F)F)cnc2Nc2cccc(C(F)(F)F)c2C)c(C)n1] | MS (m/z) 485.3 (M + H)⁺ | 6- meth- oxy-2- methyl pyridin- 3- amine | 2-((2-methyl- 3- (trifluoromethyl)- phenyl)amino)- 5- (trifluoromethyl)- nicotinic acid |
| 427 | 2-((3-chloro-2- methylphenyl)- amino)-N-(6- methoxy-2- methylpyridin-3- yl)-5- (trifluoromethyl)- nicotinamide | 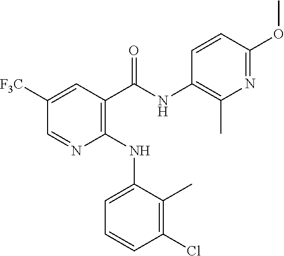 [SMILES: COc1ccc(NC(=O)c2cc(C(F)(F)F)cnc2Nc2cccc(Cl)c2C)c(C)n1] | MS (m/z) 451.2 (M + H)⁺ | 6- meth- oxy-2- methyl pyridin- 3- amine | 2-((3-chloro-2- methylphenyl) amino)-5- (trifluoromethyl)- nicotinic acid |
| 428 | 2-((2-ethyl-4- fluorophenyl)- amino)-N-(6- methoxy-2- methylpyridin-3- yl)-5- (trifluoromethyl)- nicotinamide | 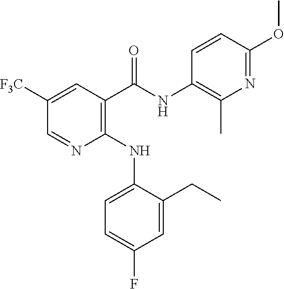 [SMILES: CCc1cc(F)ccc1Nc1ncc(C(F)(F)F)cc1C(=O)Nc1ccc(OC)nc1C] | MS (m/z) 449.3 (M + H)⁺ | 6- meth- oxy-2- methyl pyridin- 3- amine | 2-((2-ethyl-4- fluorophenyl)- amino)-5- (trifluoromethyl)- nicotinic acid |
| 429 | 2-((3,4-difluoro- 2- methylphenyl)- amino)-N-(6- methoxy-2- methylpyridin-3- yl)-5- (trifluoromethyl)- nicotinamide | 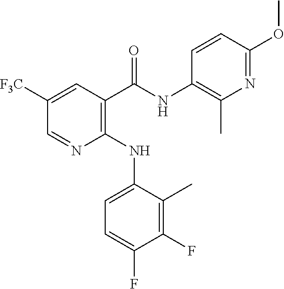 [SMILES: COc1ccc(NC(=O)c2cc(C(F)(F)F)cnc2Nc2ccc(F)c(F)c2C)c(C)n1] | MS (m/z) 453.3 (M + H)⁺ | 6- meth- oxy-2- methyl pyridin- 3- amine | 2-((3,4- difluoro-2- methylphenyl) amino)-5- (trifluoromethyl)- nicotinic acid |
| 430 | 2-((4-fluoro-2- isopropylphenyl) amino)-N-(6- methoxy-2- methylpyridin-3- yl)-5- (trifluoromethyl)- benzamide | 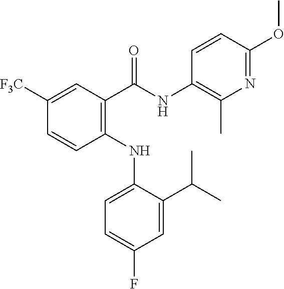 [SMILES: COc1ccc(NC(=O)c2cc(C(F)(F)F)ccc2Nc2ccc(F)cc2C(C)C)c(C)n1] | MS (m/z) 462.3 (M + H)⁺ | 6- meth- oxy-2- methyl pyridin- 3- amine | 2-((4-fluoro-2- isopropylphen- yl)amino)-5- (trifluoromethyl)- benzoic acid |
| 431 | 2-((4-fluoro-2- methylphenyl)- amino)-N-(6- methoxy-4- methylpyridin-3- yl)-5- (trifluoromethyl)- benzamide | 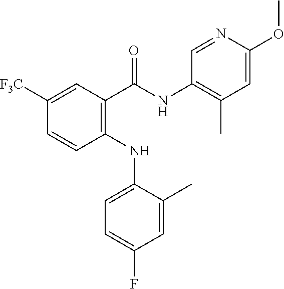 [SMILES: COc1cc(C)c(NC(=O)c2cc(C(F)(F)F)ccc2Nc2ccc(F)cc2C)cn1] | MS (m/z) 434.2 (M + H)⁺ | 6- meth- oxy-4- methyl pyridin- 3- amine | 2-((4-fluoro-2- methylphenyl) amino)-5- (trifluoromethyl)- benzoic acid |
| 432 | 6- (difluoromethoxy)- 2-((4-fluoro-2- methylphenyl)- amino)-N-(6- methoxy-2- methylpyridin-3- yl)nicotinamide | 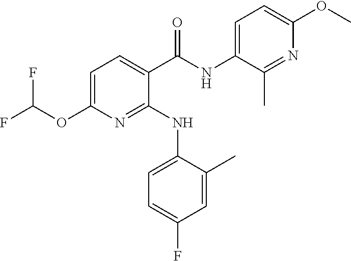 [SMILES: COc1ccc(NC(=O)c2ccc(OC(F)F)nc2Nc2ccc(F)cc2C)c(C)n1] | MS (m/z) 433 (M + H)⁺ | 6- meth- oxy-2- methyl pyridin- 3- amine | 6- (difluoro- methoxy)- 2-((4- fluoro-2- methylphenyl) amino)nicotinic acid |
| 433 | N-(6-methoxy-2- methylpyridin-3- yl)-2-((2-(2,2,2- trifluoroethyl)- phenyl)amino)-5- (trifluoromethyl)- benzamide | 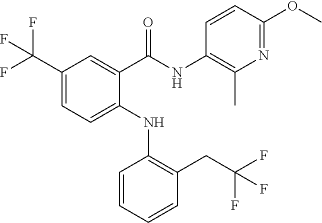 [SMILES: COc1ccc(NC(=O)c2cc(C(F)(F)F)ccc2Nc2ccccc2CC(F)(F)F)c(C)n1] | MS (m/z) 484 (M + H)⁺ | 6- meth- oxy-2- methyl pyridin- 3- amine | 2-((2-(2,2,2- trifluoroethyl)p henyl)amino)- 5- (trifluoromethyl)- benzoic acid |
| 434 | 2-((4-fluoro-2- methylphenyl)- amino)-N-(6- nitropyridin-3-yl)- 4- (trifluoromethyl)- benzamide | 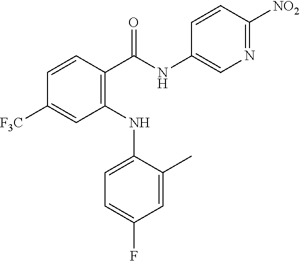 [SMILES: Cc1cc(F)ccc1Nc1cc(C(F)(F)F)ccc1C(=O)Nc1ccc([N+](=O)[O-])nc1] | MS (m/z) 435.2 (M + H)⁺ | 6-nitro- pyridin-3- amine | 2-((4-fluoro-2- methylphenyl) amino)-4- (trifluoromethyl)- benzoic acid |
| 435 | 5-fluoro-2-((4- fluoro-2- isopropylphenyl) amino)-N-(6- methoxy-2- methylpyridin-3- yl)benzamide | 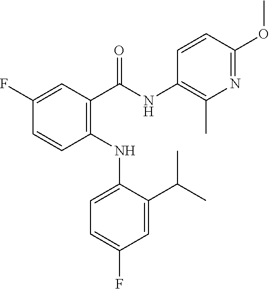 [SMILES: COc1ccc(NC(=O)c2cc(F)ccc2Nc2ccc(F)cc2C(C)C)c(C)n1] | MS (m/z) 412.4 (M + H)⁺ | 6- methoxy- 2- methyl pyridin- 3- amine | 5-fluoro-2-((4- fluoro-2- isopropylphen- yl)amino)benzoic acid |
| 436 | 2-((2- cyclopropyl-4- fluorophenyl)- amino)-N-(6- methoxy-2- methylpyridin-3- yl)benzamide | 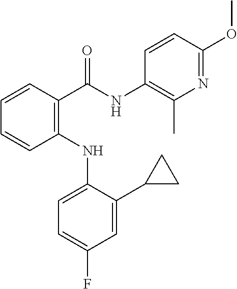 [SMILES: COc1ccc(NC(=O)c2ccccc2Nc2ccc(F)cc2C2CC2)c(C)n1] | MS (m/z) 392.3 (M + H)⁺ | 6- meth- oxy-2- methyl pyridin- 3- amine | 2-((2- cyclopropyl-4- fluorophenyl)- amino)benzoic acid |
| 437 | 2-((2- cyclopropyl-4- fluorophenyl)- amino)-N-(6- methoxy-2- methylpyridin-3- yl)-4- (trifluoromethyl)- benzamide | 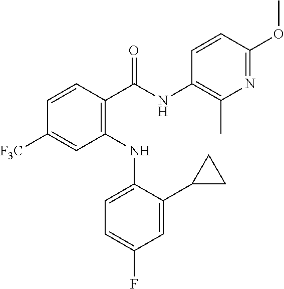 [SMILES: COc1ccc(NC(=O)c2ccc(C(F)(F)F)cc2Nc2ccc(F)cc2C2CC2)c(C)n1] | MS (m/z) 460.3 (M + H)⁺ | 6- meth- oxy-2- methyl pyridin- 3- amine | 2-((2- cyclopropyl-4- fluorophenyl)- amino)-4- (trifluoromethyl)- benzoic acid |
| 438 | 2-fluoro-6-((4- fluoro-2- methylphenyl)- amino)-N-(6- methoxy-2- methylpyridin-3- yl)benzamide | 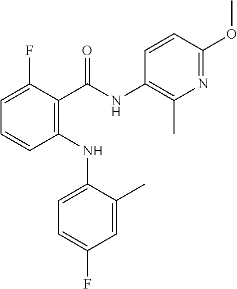 [SMILES: COc1ccc(NC(=O)c2c(F)cccc2Nc2ccc(F)cc2C)c(C)n1] | MS (m/z) 384.3 (M + H)⁺ | 6- meth- oxy-2- methyl pyridin- 3- amine | 2-fluoro-6-((4- fluoro-2- methylphenyl) amino)benzoic acid |
| 439 | 2-((2-ethyl-4- fluorophenyl)- amino)-N-(6- methoxy-2- methylpyridin-3- yl)-4- (trifluoromethyl)- benzamide | 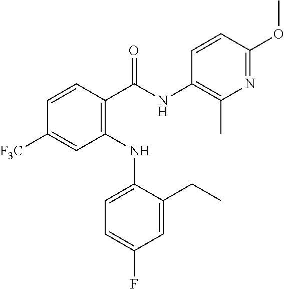 [SMILES: CCc1cc(F)ccc1Nc1cc(C(F)(F)F)ccc1C(=O)Nc1ccc(OC)nc1C] | MS (m/z) 448.3 (M + H)⁺ | 6- meth- oxy-2- methyl pyridin- 3- amine | 2-((2-ethyl-4- fluorophenyl)- amino)-4- (trifluoromethyl)- benzoic acid |
| 440 | 4- (difluoromethoxy)- 2-((4-fluoro-2- methylphenyl)- amino)-N-(6- methoxy-2- methylpyridin-3- yl)benzamide | 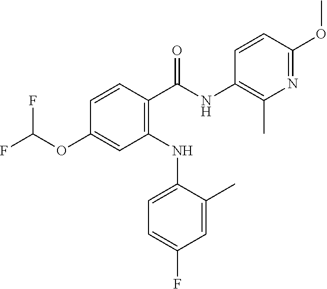 [SMILES: COc1ccc(NC(=O)c2ccc(OC(F)F)cc2Nc2ccc(F)cc2C)c(C)n1] | MS (m/z) 432.3 (M + H)⁺ | 6- meth- oxy-2- methyl pyridin- 3- amine | 4- (difluoro- methoxy)- 2-((4- fluoro-2- methylphenyl) amino)benzoic acid |
| 441 | 4-cyclopropyl-2- ((4-fluoro-2- methylphenyl)- amino)-N-(6- methoxy-2- methylpyridin-3- yl)benzamide | 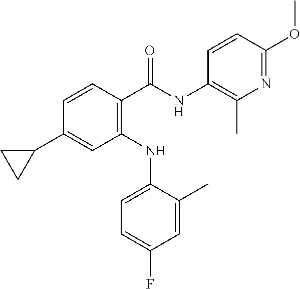 [SMILES: COc1ccc(NC(=O)c2ccc(C3CC3)cc2Nc2ccc(F)cc2C)c(C)n1] | MS (m/z) 406.4 (M + H)⁺ | 6- meth- oxy-2- methyl pyridin- 3- amine | 4-cyclopropyl- 2-((4-fluoro-2- methylphenyl) amino)benzoic acid |
| 442 | 2-((4-fluoro-2- methylphenyl)- amino)-N-(6- methoxy-2- methylpyridin-3- yl)-5- (trifluoromethoxy)- benzamide | 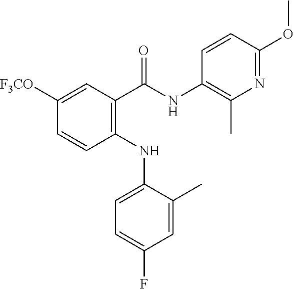 [SMILES: COc1ccc(NC(=O)c2cc(OC(F)(F)F)ccc2Nc2ccc(F)cc2C)c(C)n1] | MS (m/z) 450.3 (M + H)⁺ | 6- meth- oxy-2- methyl pyridin- 3- amine | 2-((4-fluoro-2- methylphenyl) amino)-5- (trifluorometh- oxy)benzoic acid |
| 443 | 2-((4- (difluoromethoxy)- 2-methylphenyl)- amino)-N-(6- methoxy-2- methylpyridin-3- yl)-5- (trifluoromethyl)- benzamide | 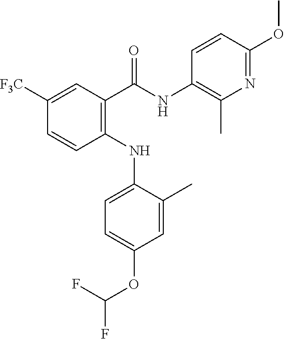 [SMILES: COc1ccc(NC(=O)c2cc(C(F)(F)F)ccc2Nc2ccc(OC(F)F)cc2C)c(C)n1] | MS (m/z) 482.3 (M + H)⁺ | 6- meth- oxy-2- methyl pyridin- 3- amine | 2-((4- (difluorometh- oxy)-2- methylphenyl) amino)-5- (trifluoromethyl)- benzoic acid |
| 444 | N-(2-ethyl-6- methoxypyridin- 3-yl)-2-((4-fluoro- 2- methylphenyl)- amino)-4- (trifluoromethoxy)- benzamide | 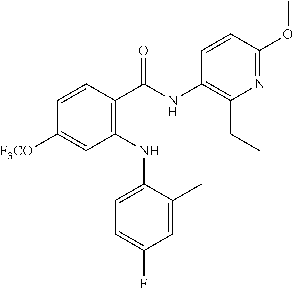 [SMILES: CCc1nc(OC)ccc1NC(=O)c1ccc(OC(F)(F)F)cc1Nc1ccc(F)cc1C] | MS (m/z) 464.4 (M + H)⁺ | 2-ethyl- 6- meth- oxypyridin- 3- amine | 2-((4-fluoro-2- methylphenyl) amino)-4- (trifluorometh- oxy)benzoic acid |
| 445 | 5-chloro-2-((2- ethyl-4- fluorophenyl)- amino)-4-fluoro-N- (6-methoxy-2- methylpyridin-3- yl)benzamide | 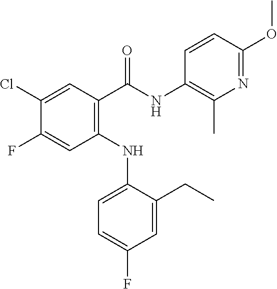 [SMILES: CCc1cc(F)ccc1Nc1cc(F)c(Cl)cc1C(=O)Nc1ccc(OC)nc1C] | MS (m/z) 432.3 (M + H)⁺ | 6- meth- oxy-2- methyl pyridin- 3- amine | 5-chloro-2-((2- ethyl-4- fluorophenyl)- amino)-4- fluorobenzoic acid |
| 446 | 2-((4-fluoro-2- methylphenyl)- amino)-N-(2- methoxy-6- ((tetrahydro-2H- pyran-2- yl)oxy)pyridin-3- yl)-4- (trifluoromethyl)- benzamide | 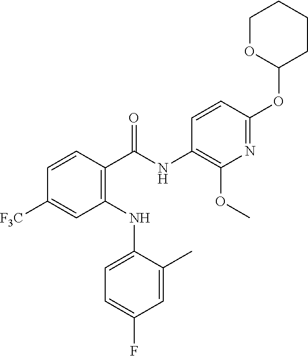 [SMILES: COc1nc(OC2CCCCO2)ccc1NC(=O)c1ccc(C(F)(F)F)cc1Nc1ccc(F)cc1C] | MS (m/z) 520.3 (M + H)⁺ | 2- meth- oxy-6- ((tetrahydro- 2H- pyran- 2-yl)oxy)- pyridin-3- amine | 2-((4-fluoro-2- methylphenyl) amino)-4- (trifluoromethyl)- benzoic acid |
| 447 | 5-chloro-2-((2- ethyl-4- fluorophenyl)- amino)-4-fluoro-N- (3-methylpyridin- 4-yl)benzamide | 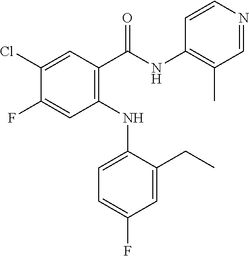 [SMILES: CCc1cc(F)ccc1Nc1cc(F)c(Cl)cc1C(=O)Nc1ccncc1C] | MS (m/z) 402.4 (M + H)⁺ | 3- amine 3- methyl pyridin- 4- amine | 5-chloro-2-((2- ethyl-4- fluorophenyl)- amino)-4- fluorobenzoic acid |
| 448 | 5-chloro-4- fluoro-2-((2- methyl-4- (trifluoromethoxy)- phenyl)amino)- N-(3- methylpyridin-4- yl)benzamide | 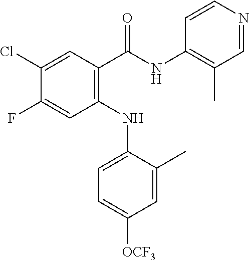 [SMILES: Cc1cnccc1NC(=O)c1cc(Cl)c(F)cc1Nc1ccc(OC(F)(F)F)cc1C] | MS (m/z) 454.3 (M + H)⁺ | 3- methyl pyridin- 4- amine | 5-chloro-4- fluoro-2-((2- methyl-4- (trifluorometh- oxy)phenyl)- amino)benzoic acid |
| 449 | 3-chloro-2,4- difluoro-6-((4- fluoro-2- methylphenyl)- amino)-N-(6- methoxy-2- methylpyridin-3- yl)benzamide | 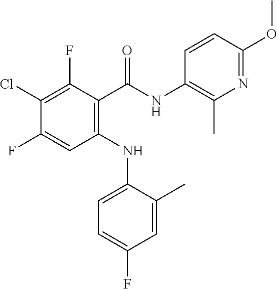 [SMILES: COc1ccc(NC(=O)c2c(Nc3ccc(F)cc3C)cc(F)c(Cl)c2F)c(C)n1] | MS (m/z) 436.2 (M + H)⁺ | 6- meth- oxy-2- methyl pyridin- 3- amine | 3-chloro-2,4- difluoro-6-((4- fluoro-2- methylphenyl) amino)benzoic acid |
| 450 | 3-chloro-4- cyano-2-fluoro- 6-((4-fluoro-2- methylphenyl)- amino)-N-(6- methoxy-2- methylpyridin-3- yl)benzamide | 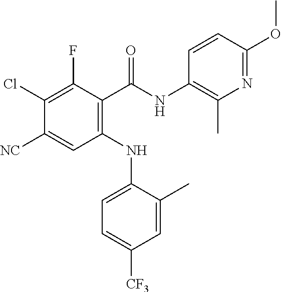 | MS (m/z) 443.3 (M + H)⁺ | 6- meth- oxy-2- methyl pyridin- 3- amine | 3-chloro-4- cyano-2- fluoro-6-((4- fluoro-2- methylphenyl) amino)benzoic acid |
| 451 | N-(6-methoxy-2- methylpyridin-3- yl)-2-((2-methyl- 4- (trifluoromethyl)- phenyl)amino)-5- (trifluoromethyl)- nicotinamide | 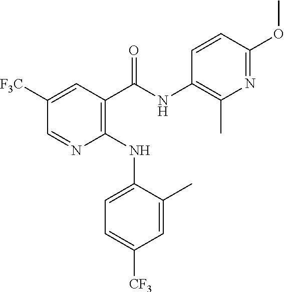 [SMILES: COc1ccc(NC(=O)c2cc(C(F)(F)F)cnc2Nc2ccc(C(F)(F)F)cc2C)c(C)n1] | MS (m/z) 485.3 (M + H)⁺ | 6- meth- oxy-2- methyl pyridin- 3- amine | 2-((2-methyl- 4- (trifluoromethyl)- phenyl)amino)- 5- (trifluoromethyl)- nicotinic acid |
| 452 | 2-((4-chloro-2- methylphenyl)- amino)-N-(6- methoxy-2- methylpyridin-3- yl)-5- (trifluoromethyl)- nicotinamide | 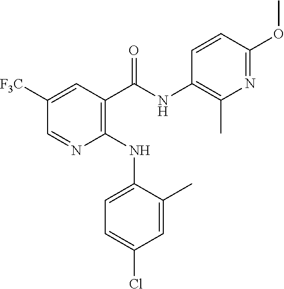 [SMILES: COc1ccc(NC(=O)c2cc(C(F)(F)F)cnc2Nc2ccc(Cl)cc2C)c(C)n1] | MS (m/z) 451.3 (M + H)⁺ | 6- meth- oxy-2- methyl pyridin- 3- amine | 2-((4-chloro-2- methylphenyl) amino)-5- (trifluoromethy IJnicotinic acid |
| 453 | 2-((4-fluoro-2- methylphenyl)- amino)-N-(4- (methylsulfonyl)- phenyl)-4- (trifluoromethyl)- benzamide | 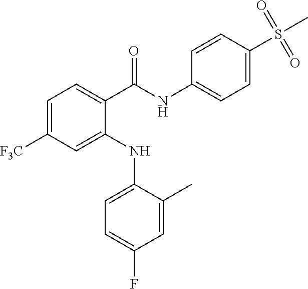 [SMILES: Cc1cc(F)ccc1Nc1cc(C(F)(F)F)ccc1C(=O)Nc1ccc(S(C)(=O)=O)cc1] | MS (m/z) 467.2 (M + H)⁺ | 4- (methyl sulfonyl)- aniline | 2-((4-fluoro-2- methylphenyl) amino)-4- (trifluoromethyl)- benzoic acid |
| 454 | 2-((4-fluoro-2- methylphenyl)- amino)-N-(3- (methylsulfonyl)- phenyl)-4- (trifluoromethyl)- benzamide | 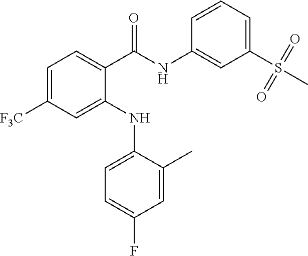 [SMILES: Cc1cc(F)ccc1Nc1cc(C(F)(F)F)ccc1C(=O)Nc1cccc(S(C)(=O)=O)c1] | MS (m/z) 467.2 (M + H)⁺ | 3- (methyl sulfonyl)- aniline | 2-((4-fluoro-2- methylphenyl) amino)-4- (trifluoromethyl)- benzoic acid |
| 455 | methyl 4-(2-((4- fluoro-2- methylphenyl)- amino)-4- (trifluoromethyl)- benzamido)benzo- ate | 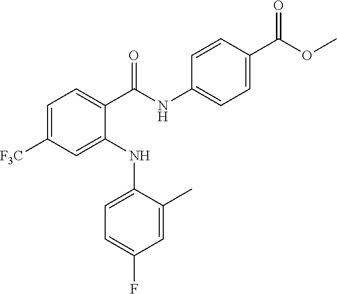 [SMILES: COC(=O)c1ccc(NC(=O)c2ccc(C(F)(F)F)cc2Nc2ccc(F)cc2C)cc1] | MS (m/z) 447.3 (M + H)⁺ | methyl 4- amino- benzoate | 2-((4-fluoro-2- methylphenyl) amino)-4- (trifluoromethyl)- benzoic acid |
| 456 | methyl 3-(2-((4- fluoro-2- methylphenyl)- amino)-4- (trifluoromethyl)- benzamido)benzo- ate | 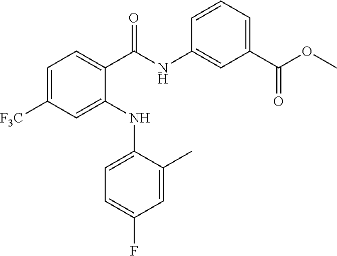 [SMILES: COC(=O)c1cccc(NC(=O)c2ccc(C(F)(F)F)cc2Nc2ccc(F)cc2C)c1] | MS (m/z) 447.3 (M + H)⁺ | methyl 3- amino- benzoate | 2-((4-fluoro-2- methylphenyl) amino)-4- (trifluoromethyl)- benzoic acid |
| 457 | 2-((4-fluoro-2- methylphenyl)- amino)-N-(pyridin- 3-yl)-4- (trifluoromethyl)- tenzamide | 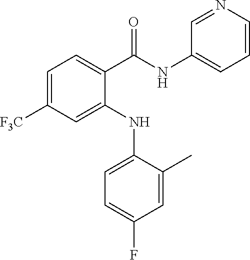 [SMILES: Cc1cc(F)ccc1Nc1cc(C(F)(F)F)ccc1C(=O)Nc1cccnc1] | MS (m/z) 390.3 (M + H)⁺ | pyridin- 3- amine | 2-((4-fluoro-2- methylphenyl) amino)-4- (trifluoromethyl)- benzoic aci |
| 458 | 2-((4-fluoro-2- methylphenyl)- amino)-N-(2- methoxypyrimidin- 5-yl)-4- (trifluoromethyl)- benzamide | 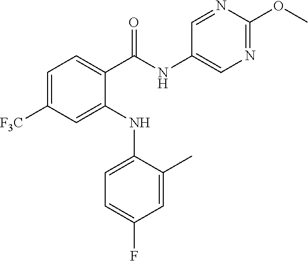 [SMILES: COc1ncc(NC(=O)c2ccc(C(F)(F)F)cc2Nc2ccc(F)cc2C)cn1] | MS (m/z) 421.3 (M + H)⁺ | 2- methoxy- pyrimidin-5- amine | 2-((4-fluoro-2- methylphenyl) amino)-4- (trifluoromethyl)- benzoic acid |
| 459 | 2-((2-bromo-4- fluorophenyl)- amino)-5-chloro-N- (6-methoxy-2- methylpyridin-3- yl)benzamide | 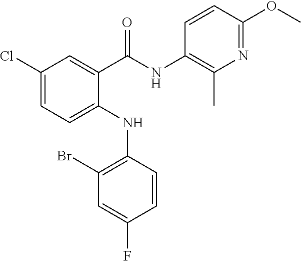 [SMILES: COc1ccc(NC(=O)c2cc(Cl)ccc2Nc2ccc(F)cc2Br)c(C)n1] | MS (m/z) 466.2 (M+3H)⁺ | 6- meth- oxy-2- methyl pyridin- 3- amine | 2-((2-bromo- 4- fluorophenyl)- amino)-5- chloro benzoic acid |
| 460 | 2-((4-fluoro-2- methylphenyl)- amino)-N-(2- methoxypyridin- 3-yl)-4- (trifluoromethyl)- benzamide | 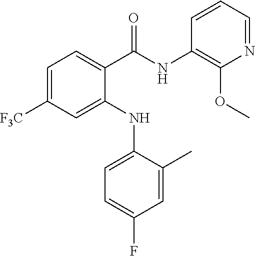 [SMILES: COc1ncccc1NC(=O)c1ccc(C(F)(F)F)cc1Nc1ccc(F)cc1C] | MS (m/z) 420.3 (M + H)⁺ | 2- meth- oxypyridin- 3- amine | 2-((4-fluoro-2- methylphenyl) amino)-4- (trifluoromethyl)- benzoic acid |
| 461 | 2-((4-fluoro-2- methylphenyl)- amino)-N-(2- methoxy-4- methylpyrimidin- 5-yl)-4- (trifluoromethoxy)- benzamide | 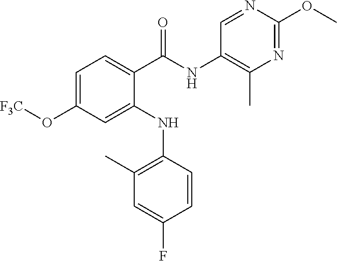 [SMILES: COc1ncc(NC(=O)c2ccc(OC(F)(F)F)cc2Nc2ccc(F)cc2C)c(C)n1] | MS (m/z) 451.4 (M + H)⁺ | 2- meth- oxy-4- methyl pyrimidin- 5- amine | 2-((4-fluoro-2- methylphenyl) amino)-4- (trifluorometh- oxy)benzoic acid |
| 462 | 2-((4-fluoro-2- methylphenyl)- amino)-N-(6- methoxy-2- methylpyridin-3- yl)-6- methylnicotin- amide | 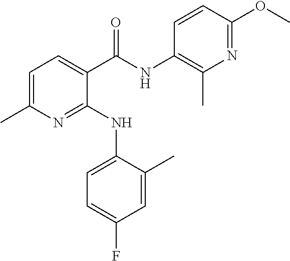 | MS (m/z) 381.4 (M + H)⁺ | 6- meth- oxy-2- methyl pyridin- 3- amine | 2-((4-fluoro-2- methylphenyl) amino)-6- methylnicotinic acid |
| 463 | 5-chloro-6- cyano-2-((2- ethyl-4- fluorophenyl)- amino)-N-(6- methoxy-2- methylpyridin-3- yl)nicotinamide | 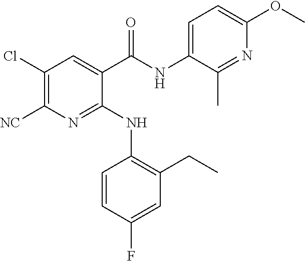 [SMILES: CCc1cc(F)ccc1Nc1nc(C#N)c(Cl)cc1C(=O)Nc1ccc(OC)nc1C] | MS (m/z) 440.3 (M + H)⁺ | 6- meth- oxy-2- methyl pyridin- 3- amine | 5-chloro-6- cyano-2-((2- ethyl-4- fluorophenyl)- amino)nicotinic acid |
| 464 | 2-((4-fluoro-2- methylphenyl)- amino)-N-(pyridin- 3-yl)-5- (trifluoromethyl)- benzamide | 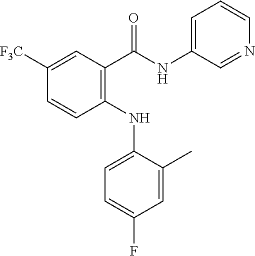 [SMILES: Cc1cc(F)ccc1Nc1ccc(C(F)(F)F)cc1C(=O)Nc1cccnc1] | MS (m/z) 390.3 (M + H)⁺ | pyridin- 3- amine | 2-((4-fluoro-2- methylphenyl) amino)-5- (trifluoromethyl)- benzoic acid |
| 465 | 2-((4-fluoro-2- methylphenyl)- amino)-N-(4- methylpyridin-3- yl)-5- (trifluoromethyl)- benzamide | 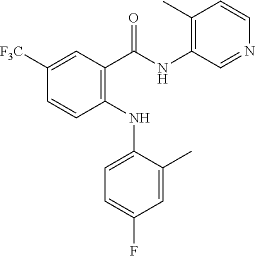 [SMILES: Cc1ccncc1NC(=O)c1cc(C(F)(F)F)ccc1Nc1ccc(F)cc1C] | MS (m/z) 404.3 (M + H)⁺ | 4- methyl pyridin- 3- amine | 2-((4-fluoro-2- methylphenyl) amino)-5- (trifluoromethyl)- benzoic acid |
| 466 | 2-((4-fluoro-2- methylphenyl)- amino)-N- (pyridazin-4-yl)- 5- (trifluoromethyl)- benzamide | 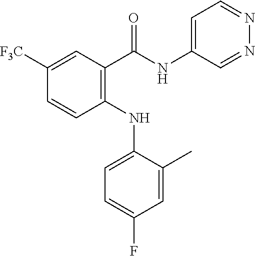 [SMILES: Cc1cc(F)ccc1Nc1ccc(C(F)(F)F)cc1C(=O)Nc1ccnnc1] | MS (m/z) 391.3 (M + H)⁺ | pyridazin-4- amine | 2-((4-fluoro-2- methylphenyl) amino)-5- (trifluoromethyl)- benzoic acid |
| 467 | 2-((4-fluoro-2- methylphenyl)- amino)-N-(4- hydroxy-2- methylphenyl)-5- (trifluoromethyl)- tenzamide | 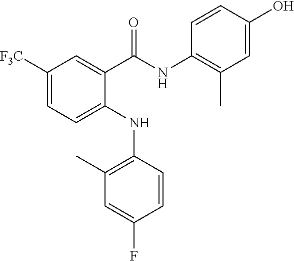 [SMILES: Cc1cc(O)ccc1NC(=O)c1cc(C(F)(F)F)ccc1Nc1ccc(F)cc1C] | MS (m/z) 419.3 (M + H)⁺ | 4- amino- 3- methyl phenol | 2-((4-fluoro-2- methylphenyl) amino)-5- (trifluoromethyl)- benzoic acid |
| 468 | N-(4-((tert- butyldimethylsilyl)- oxy)-2- methylphenyl)-2- ((4-fluoro-2- methylphenyl)- amino)-4- (trifluoromethyl)- benzamide | 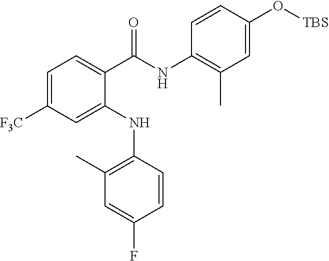 | MS (m/z) 533.3 (M + H)⁺ | 4-((tert- butyl- dimethyl- silyl)oxy)- 2- methylaniline | 2-((4-fluoro-2- methylphenyl) amino)-4- (trifluoromethyl)- benzoic acid |
| 469 | N-(1,1- dioxidotetrahy- drothiophen-3-yl)- 2-((4-fluoro-2- methylphenyl)- amino)-5- (trifluoromethyl)- benzamide | 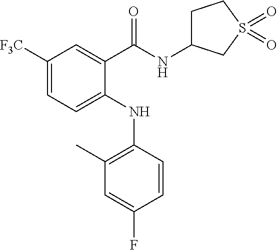 [SMILES: Cc1cc(F)ccc1Nc1ccc(C(F)(F)F)cc1C(=O)NC1CCS(=O)(=O)C1] | MS (m/z) 431.3 (M + H)⁺ | 3- amino- tetrahy- drothio- phene 1,1- dioxide, Hydro- chloride | 2-((4-fluoro-2- methylphenyl) amino)-5- (trifluoromethyl)- benzoic acid |
| 470 | 2-((3- fluorocyclopentyl)- amino)-N-(6- methoxy-2- methylpyridin-3- yl)-4- (trifiuoromethyl)- benzamide | 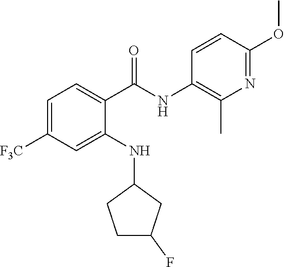 [SMILES: COc1ccc(NC(=O)c2ccc(C(F)(F)F)cc2NC2CCC(F)C2)c(C)n1] | MS (m/z) 412.4 (M + H)⁺ | 6- meth- oxy-2- methyl pyridin- 3- amine, Hydro- chloride | 2-((3- fluorocyclopen tyl)amino)-4- (trifluoromethyl)- benzoic acid |
| 471 | 2-((4-fluoro-2- methylphenyl)- amino)-N-(1- methyl-1H- pyrazol-5-yl)-5- (trifluoromethyl)- benzamide | 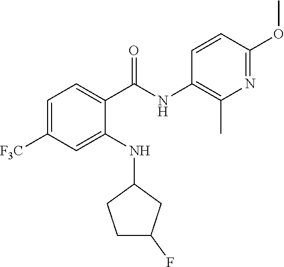 | MS (m/z) 393.4 (M + H)⁺ | 1- methyl- 1H- pyrazol- 5- amine | 2-((4-fluoro-2- methylphenyl) amino)-5- (trifluoromethyl)- benzoic acid |
| 472 | 4-chloro-5- fluoro-2-((4- fluoro-2- methylphenyl)- amino)-N-(6- methoxy-2- methylpyridin-3- yl)benzamide | 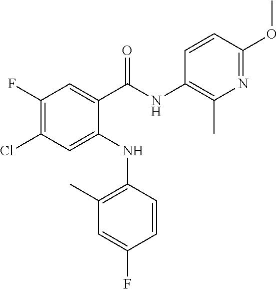 [SMILES: COc1ccc(NC(=O)c2cc(F)c(Cl)cc2Nc2ccc(F)cc2C)c(C)n1] | MS (m/z) 418.3 (M + H)⁺ | 6- meth- oxy-2- methyl pyridin- 3- amine | 4-chloro-5- fluoro-2-((4- fluoro-2- methylphenyl) amino)benzoic acid |
| 473 | tert-butyl 4-(2- ((4-fluoro-2- methylphenyl)- amino)-5- (trifluoromethyl)- benzamido)-3- methyl-1H- pyrazole-1- carboxylate | 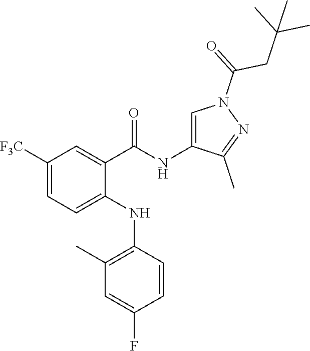 [SMILES: Cc1cc(F)ccc1Nc1ccc(C(F)(F)F)cc1C(=O)Nc1cn(C(=O)CC(C)(C)C)nc1C] | MS (m/z) 493.4 (M + H)⁺ | tert- butyl 4- amino- 3- methyl- 1H- pyrazole-1- carboxylate | 2-((4-fluoro-2- methylphenyl) amino)-5- (trifluoromethyl)- benzoic acid |
| 474 | N-(1- acetylpiperidin- 4-yl)-2-((4-fluoro- 2- methylphenyl)- amino)-5- (trifluoromethyl)- benzamide | 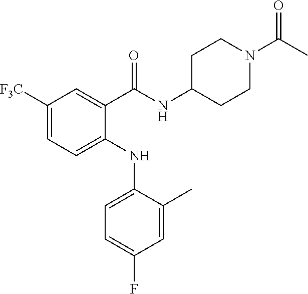 [SMILES: CC(=O)N1CCC(NC(=O)c2cc(C(F)(F)F)ccc2Nc2ccc(F)cc2C)CC1] | MS (m/z) 438.4 (M + H)⁺ | 1-(4- amino- piperidin- 1- yl)ethan-1-one | 2-((4-fluoro-2- methylphenyl) amino)-5- (trifluoromethyl)- benzoic acid |
| 475 | 2-((4-fluoro-2- methylphenyl)- amino)-N- (pyridazin-4-yl)- 5- (trifluoromethyl)- benzamide | 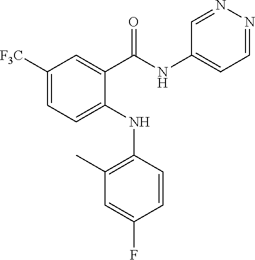 [SMILES: Cc1cc(F)ccc1Nc1ccc(C(F)(F)F)cc1C(=O)Nc1ccnnc1] | MS (m/z) 391.3 (M + H)⁺ | pyridazin-4- amine | 2-((4-fluoro-2- methylphenyl) amino)-5- (trifluoromethyl)- benzoic acid |
| 476 | 2-((4-fluoro-2- methylphenyl)- amino)-N-(5- methylpyridin-3- yl)-5- (trifluoromethyl)- benzamide | 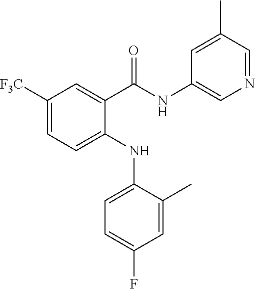 [SMILES: Cc1cncc(NC(=O)c2cc(C(F)(F)F)ccc2Nc2ccc(F)cc2C)c1] | MS (m/z) 404.4 (M + H)⁺ | 5- methyl pyridin- 3- amine | 2-((4-fluoro-2- methylphenyl) amino)-5- (trifluoromethyl)- benzoic acid |
| 477 | 2-((4-fluoro-2- methylphenyl)- amino)-N-(2- methylpyridin-3- yl)-5- (trifluoromethyl)- benzamide | 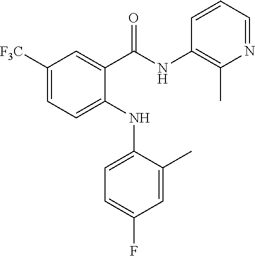 [SMILES: Cc1cc(F)ccc1Nc1ccc(C(F)(F)F)cc1C(=O)Nc1cccnc1C] | MS (m/z) 404.4 (M + H)⁺ | 2- methyl pyridin- 3- amine | 2-((4-fluoro-2- methylphenyl) amino)-5- (trifluoromethyl)- benzoic acid |
| 478 | tert-butyl 4-(2- ((4-fluoro-2- methylphenyl)- amino)-5- (trifluoromethyl)- benzamido)-1H- pyrazole-1- carboxylate | 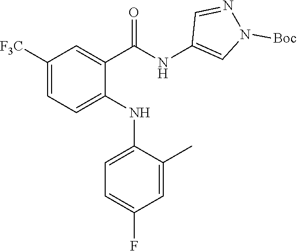 [SMILES: Cc1cc(F)ccc1Nc1ccc(C(F)(F)F)cc1C(=O)Nc1cnn(C(=O)OC(C)(C)C)c1] | MS (m/z) 379.4 (M-100 + H)⁺ | butyl 4- amino- 1H- pyrazol e-1- carboxy late | 2-((4-fluoro-2- methylphenyl) amino)-5- (trifluoromethyl)- benzoic acid |
| 479 | N-(1,1- dioxidotetrahy- dro-2H-thiopyran- 4-yl)-2-((4-f)uoro- 2- methylphenyl)- amino)-4- (trifluoromethyl)- benzamide | 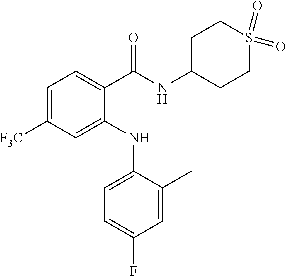 [SMILES: Cc1cc(F)ccc1Nc1cc(C(F)(F)F)ccc1C(=O)NC1CCS(=O)(=O)CC1] | MS (m/z) 445.3 (M + H)⁺ | 4- aminot etrahyd ro-2H- thiopyr an 1,1- dioxide | 2-((4-fluoro-2- methylphenyl) amino)-4- (trifluoromethyl)- benzoic acid |
| 480 | N-(2-chloro-6- methoxypyridin- 3-yl)-2-((4-fluoro- 2- methylphenyl)- amino)-5- (trifluoromethyl)- benzamide | 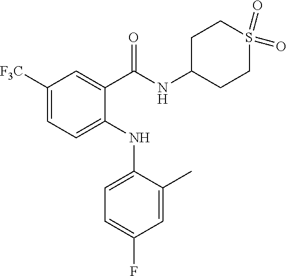 | MS (m/z) 454.1 (M + H)⁺ | 2- chloro- 6- meth- oxypyridin- 3- amine, Hydro- chloride | 2-((4-fluoro-2- methylphenyl) amino)-5- (trifluoromethyl)- benzoic acid |
| 481 | 2-((4-fluoro-2- methylphenyl)- amino)-N-(3- methylpyridin-4- yl)-5- (trifluoromethyl)- benzamide | 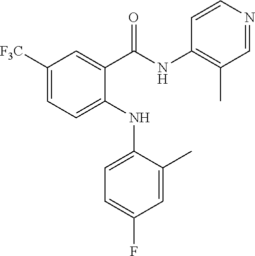 [SMILES: Cc1cnccc1NC(=O)c1cc(C(F)(F)F)ccc1Nc1ccc(F)cc1C] | MS (m/z) 404.3 (M + H)⁺ | 3- methyl pyridin- 4- amine | 2-((4-fluoro-2- methylphenyl) amino)-5- (trifluoromethyl)- benzoic acid |
| 482 | 2-((4-fluoro-2- methylphenyl)- amino)-N- ((2R,3S)-2- methyl-6- oxopiperidin-3- yl)-5- (trifluoromethyl)- benzamide | 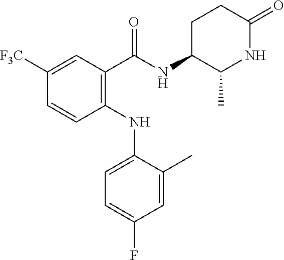 [SMILES: Cc1cc(F)ccc1Nc1ccc(C(F)(F)F)cc1C(=O)N[C@H]1CCC(=O)N[C@@H]1C] | MS (m/z) 424.1 (M + H)⁺ | 5- amino- 6- methyl piperidin- 2-one | 2-((4-fluoro-2- methylphenyl) amino)-5- (trifluoromethyl)- benzoic acid |
| 483 | N-(2-bromo-6- methoxy-4- methylpyridin-3- yl)-2-((4-fluoro-2- methylphenyl)- amino)-4- (trifluoromethyl)- benzamide | 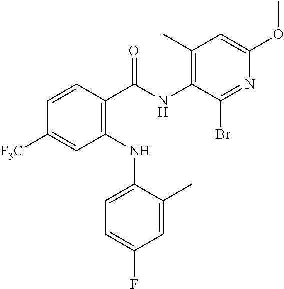 [SMILES: COc1cc(C)c(NC(=O)c2ccc(C(F)(F)F)cc2Nc2ccc(F)cc2C)c(Br)n1] | MS (m/z) 514.1 (M + H)⁺ | 2- bromo- 6- meth- oxy-4- methyl pyridin- 3- amine | 2-((4-fluoro-2- methylphenyl) amino)-4- (trifluoromethyl)- benzoic acid |
| 484 | 2-((4-fluoro-2- isopropylphenyl) amino)-N-(6- methoxy-2- methylpyridin-3- yl)-6- (trifluoromethyl)- nicotinamide | 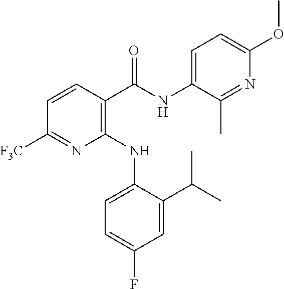 [SMILES: COc1ccc(NC(=O)c2ccc(C(F)(F)F)nc2Nc2ccc(F)cc2C(C)C)c(C)n1] | MS (m/z) 463.3 (M + H)⁺ | 6- meth- oxy-2- methyl pyridin- 3- amine | 2-((4-fluoro-2- isopropylphen- yl)amino)-6- (trifluoromethyl)- nicotinic acid |
| 485 | 5-chloro-2-((4- fluoro-2- isopropylphenyl) amino)-N-(6- methoxy-2- methylpyridin-3- yl)nicotinamide | 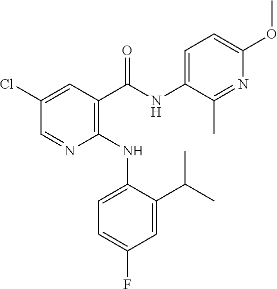 [SMILES: COc1ccc(NC(=O)c2cc(Cl)cnc2Nc2ccc(F)cc2C(C)C)c(C)n1] | MS (m/z) 429.3 (M + H)⁺ | 6- meth- oxy-2- methyl pyridin- 3- amine | 5-chloro-2-((4- fluoro-2- isopropylphen- yl)amino)nicotinic acid |
| 486 | N-(3- acetamidophenyl)- 2-((4-fluoro-2- methylphenyl)- amino)-4- (trifluoromethyl)- benzamide | 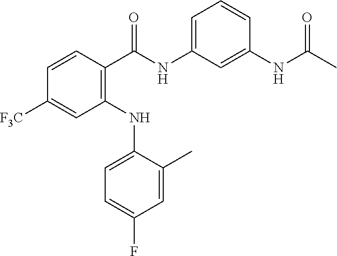 [SMILES: CC(=O)Nc1cccc(NC(=O)c2ccc(C(F)(F)F)cc2Nc2ccc(F)cc2C)c1] | MS (m/z) 446.0 (M + H)⁺ | N-(3- amino- phenyl)- acetamide | 2-((4-fluoro-2- methylphenyl) amino)-4- (trifluoromethyl)- benzoic acid |
| 487 | N-(4- acetamidophenyl)- 2-((4-fluoro-2- methylphenyl)- amino)-4- (trifluoromethyl)- benzamide | 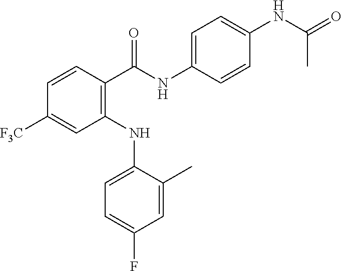 [SMILES: CC(=O)Nc1ccc(NC(=O)c2ccc(C(F)(F)F)cc2Nc2ccc(F)cc2C)cc1] | MS (m/z) 446.0 (M + H)⁺ | N-(4- amino- phenyl)- acetamide | 2-((4-fluoro-2- methylphenyl) amino)-4- (trifluoromethyl)- benzoic acid |
| 488 | 2-((4-fluoro-2- isopropylphenyl) amino)-N-(6- methoxy-2,4- dimethylpyridin- 3-yl)-4- (trifluoromethyl)- benzamide | 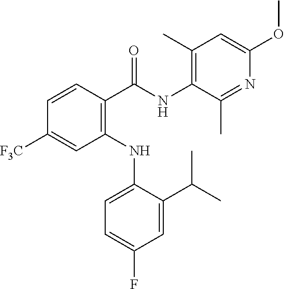 [SMILES: COc1cc(C)c(NC(=O)c2ccc(C(F)(F)F)cc2Nc2ccc(F)cc2C(C)C)c(C)n1] | MS (m/z) 476.1 (M + H)⁺ | 6- meth- oxy-2,4- dimethyl- pyridin- 3- amine | 2-((4-fluoro-2- isopropylphen- yl)amino)-4- (trifluoromethyl)- benzoic acid |
| 489 | 2-((4-fluoro-2- isopropylphenyl) amino)-N-(6- methoxy-2- methylpyridin-3- yl)-5- (trifluoromethyl)- nicotinamide | 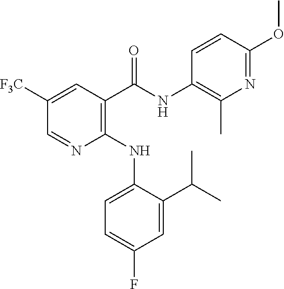 [SMILES: COc1ccc(NC(=O)c2cc(C(F)(F)F)cnc2Nc2ccc(F)cc2C(C)C)c(C)n1] | MS (m/z) 463.1 (M + H)⁺ | 6- meth- oxy-2- methyl pyridin- 3- amine | 2-((4-fluoro-2- isopropylphen- yl)amino)-5- (trifluoromethyl)- nicotinic acid |
| 490 | 2-(benzylamino)- N-(6-methoxy-2- methylpyridin-3- yl)-5- (trifluoromethyl)- benzamide | 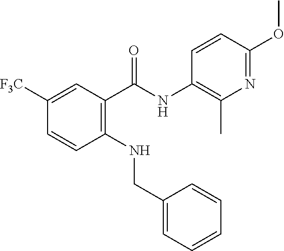 [SMILES: COc1ccc(NC(=O)c2cc(C(F)(F)F)ccc2NCc2ccccc2)c(C)n1] | MS (m/z) 416.2 (M + H)⁺ | 6- meth- oxy-2- methyl pyridin- 3- amine | 2- (benzylamino)- 5- (trifluoromethyl)- benzoic acid |
| 491 | NN-(2-ethyl-6- methoxypyridin- 3-yl)-2-((4 fluoro- 2- methylphenyl)- amino)-5- (trifluoromethyl)- benzamide | 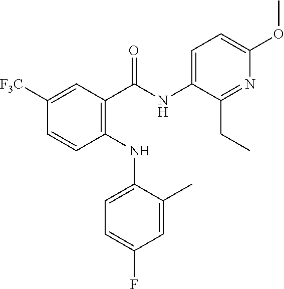 [SMILES: CCc1nc(OC)ccc1NC(=O)c1cc(C(F)(F)F)ccc1Nc1ccc(F)cc1C] | MS (m/z) 448.0 (M + H)⁺ | 2-ethyl- 6- meth- oxypyridin- 3- amine | 2-((4-fluoro-2- methylphenyl) amino)-5- (trifluoromethyl)- benzoic acid |
| 492 | 4- (dimethylamino)- 2-((4-fluoro-2- methylphenyl)- amino)-N-(6- methoxy-2- methylpyridin-3- yl)benzamide | 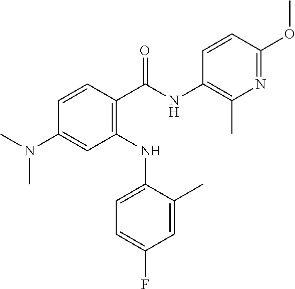 | MS (m/z) 409.1 (M + H)⁺ | 6- meth- oxy-2- methyl pyridin- 3- amine | 4- (dimethylamino)- 2-((4- fluoro-2- methylphenyl) amino)benzoic acid |
| 493 | 5-chloro-2-((2- ethoxy-4- fluorophenyl)- amino)-N-(6- methoxy-2- methylpyridin-3- yl)benzamide | 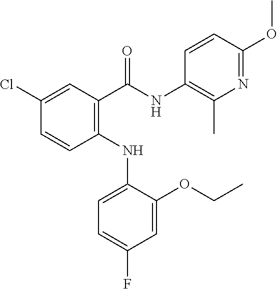 [SMILES: CCOc1cc(F)ccc1Nc1ccc(Cl)cc1C(=O)Nc1ccc(OC)nc1C] | MS (m/z) 430.0 (M + H)⁺ | 6- meth- oxy-2- methyl pyridin- 3- amine | 5-chloro-2-((2- ethoxy-4- fluorophenyl)- amino)benzoic acid |
| 494 | 2-((4-fluoro-2- isopropylphenyl) amino)-N-(6- methoxy-2- methylpyridin-3- yl)-4- methylbenzamide | 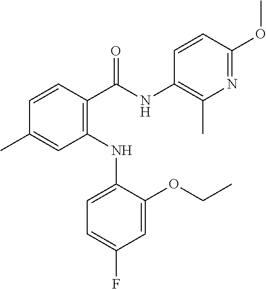 | MS (m/z) 408.2 (M + H)⁺ | 6- meth- oxy-2- methyl pyridin- 3- amine | 2-((4-fluoro-2- isopropylphen- yl)amino)-4- methylbenzoic acid |
| 495 | 4- (difluoromethyl)- 5-fluoro-2-((4- fluoro-2- methylphenyl)- amino)-N-(6- methoxy-2- methylpyridin-3- yl)benzamide | 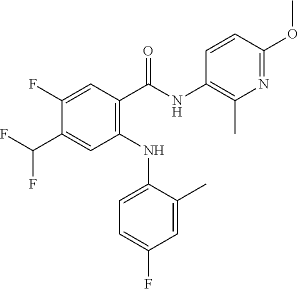 [SMILES: COc1ccc(NC(=O)c2cc(F)c(C(F)F)cc2Nc2ccc(F)cc2C)c(C)n1] | MS (m/z) 434.0 (M + H)⁺ | 6- meth- oxy-2- methyl pyridin- 3- amine | 4- (difluoromethyl)- -5-fluoro-2- ((4-fluoro-2- methylphenyl) amino)benzoic acid |
| 496 | 5-fluoro-2-((4- fluoro-2- methylphenyl)- amino)-N-(6- methoxy-2- methylpyridin-3- yl)-6- methylnicotin- amide | 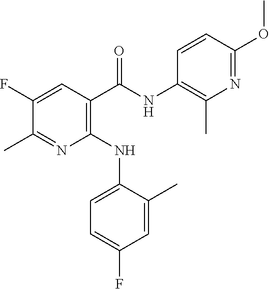 | MS (m/z) 399.2 (M + H)⁺ | 6- meth- oxy-2- methyl pyridin- 3- amine | 5-fluoro-2-((4- fluoro-2- methylphenyl) amino)-6- methylnicotinic acid |
| 497 | 5-chloro-2-((4- fluoro-2- methylphenyl)- amino)-N-(6- methoxy-2- methylpyridin-3- yl)-6- methylnicotin- amide | 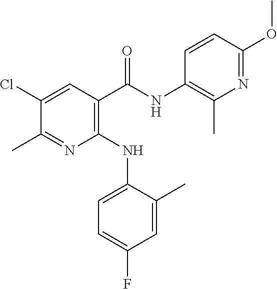 | MS (m/z) 415.0 (M + H)⁺ | 6- meth- oxy-2- methyl pyridin- 3- amine | 5-chloro-2-((4- fluoro-2- methylphenyl) amino)-6- methylnicotinic acid |
| 498 | 5,6-dichloro-2- ((4-fluoro-2- methylphenyl)- amino)-N-(6- methoxy-2- methylpyridin-3- yl)nicotinamide | 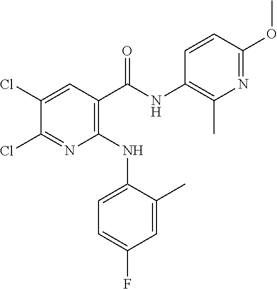 [SMILES: COc1ccc(NC(=O)c2cc(Cl)c(Cl)nc2Nc2ccc(F)cc2C)c(C)n1] | MS (m/z) 435.0 (M + H)⁺ | 6- meth- oxy-2- methyl pyridin- 3- amine | 5,6-dichloro-2- ((4-fluoro-2- methylphenyl) amino)nicotinic acid |
| 499 | 3-chloro-2- fluoro-N-(6- methoxy-2- methylpyridin-3- yl)-6-((2-methyl- 4- (trifluoromethoxy)- phenyl)amino)- benzamide | 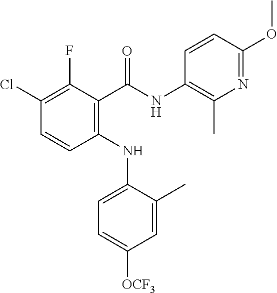 [SMILES: COc1ccc(NC(=O)c2c(Nc3ccc(OC(F)(F)F)cc3C)ccc(Cl)c2F)c(C)n1] | MS (m/z) 484.0 (M + H)⁺ | 6- meth- oxy-2- methyl pyridin- 3- amine | 3-chloro-2- fluoro-6-((2- methyl-4- (trifluorometh- oxy)phenyl)- amino)benzoic acid |
| 500 | 5-cyano-2-((4- fluoro-2- methylphenyl)- amino)-N-(6- methoxy-2- methylpyridin-3- yl)benzamide | 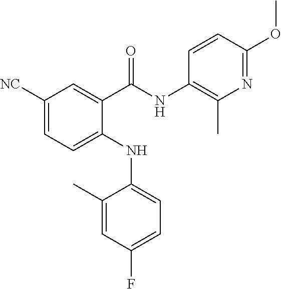 [SMILES: COc1ccc(NC(=O)c2cc(C#N)ccc2Nc2ccc(F)cc2C)c(C)n1] | MS (m/z) 391.0 (M + H)⁺ | 6- meth- oxy-2- methyl pyridin- 3- amine | 5-cyano-2-((4- fluoro-2- methylphenyl) amino)benzoic acid |
| 501 | 5-chloro-2-((4- fluoro-2- isopropylphenyl) amino)-N-(6- methoxy-2- methylpyridin-3- yl)benzamide | 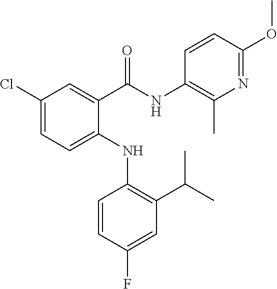 [SMILES: COc1ccc(NC(=O)c2cc(Cl)ccc2Nc2ccc(F)cc2C(C)C)c(C)n1] | MS (m/z) 428.1 (M + H)⁺ | 6- meth- oxy-2- methyl pyridin- 3- amine | 5-chloro-2-((4- fluoro-2- isopropylphen- yl)amino)benzoic acid |
| 502 | 2-((4-fluoro-3- methoxy-2- methylphenyl)- amino)-N-(6- methoxy-2- methylpyridin-3- yl)-4- (trifluoromethyl)- benzamide | 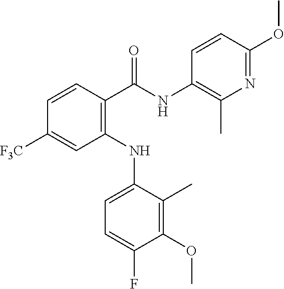 [SMILES: COc1ccc(NC(=O)c2ccc(C(F)(F)F)cc2Nc2ccc(F)c(OC)c2C)c(C)n1] | MS (m/z) 464.0 (M + H)⁺ | 6- meth- oxy-2- methyl pyridin- 3- amine | 2-((4-fluoro-3- methoxy-2- methylphenyl) amino)-4- (trifluoromethyl)- benzoic acid |
| 503 | 5-(3-chloro-2- fluoro-6-((4- fluoro-2- methylphenyl)- amino)benzamido)- picolinamide | 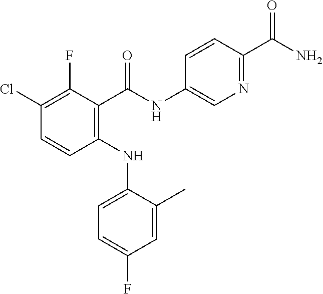 [SMILES: Cc1cc(F)ccc1Nc1ccc(Cl)c(F)c1C(=O)Nc1ccc(C(N)=O)nc1] | MS (m/z) 417.0 (M + H)⁺ | 5- aminopico- linamide | 3-chloro-2- fluoro-6-((4- fluoro-2- methylphenyl) amino)benzoic acid |
| 504 | 3-chloro-2- fluoro-6-((4- fluoro-2- methylphenyl)- amino)-N-(6- methoxypyridin- 3-yl)benzamide | 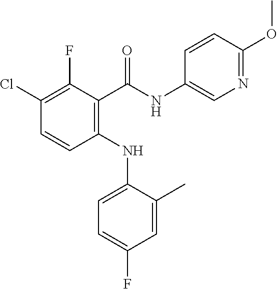 [SMILES: COc1ccc(NC(=O)c2c(Nc3ccc(F)cc3C)ccc(Cl)c2F)cn1] | MS (m/z) 404.0 (M + H)⁺ | 6- meth- oxypyridin- 3- amine | 3-chloro-2- fluoro-6-((4- fluoro-2- methylphenyl) amino)benzoic acid |
| 505 | 3-chloro-2- fluoro-6-((4- fluoro-2- methylphenyl)- amino)-N-(6- methoxy-2- methylpyridin-3- yl)benzamide | 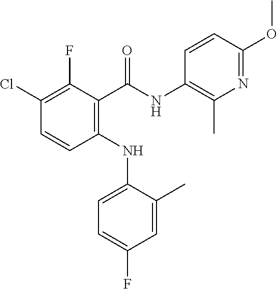 [SMILES: COc1ccc(NC(=O)c2c(Nc3ccc(F)cc3C)ccc(Cl)c2F)c(C)n1] | MS (m/z) 418.0 (M + H)⁺ | 6- meth- oxy-2- methyl pyridin- 3- amine | 3-chloro-2- fluoro-6-((4- fluoro-2- methylphenyl) amino)benzoic acid |
| 506 | 4-(3-chloro-2- fluoro-6-((4- fluoro-2- methylphenyl)- amino)benzamido)- picolinamide | 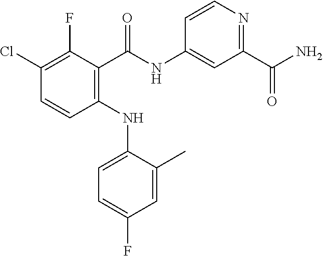 [SMILES: Cc1cc(F)ccc1Nc1ccc(Cl)c(F)c1C(=O)Nc1ccnc(C(N)=O)c1] | MS (m/z) 417.0 (M + H)⁺ | 4- amino- picolinamide | 3-chloro-2- fluoro-6-((4- fluoro-2- methylphenyl) amino)benzoic acid |
| 507 | N-(6-methoxy-2- methylpyridin-3- yl)-2-((3- methylthiophen- 2-yl)amino)-5- (trifluoromethyl)- benzamide | 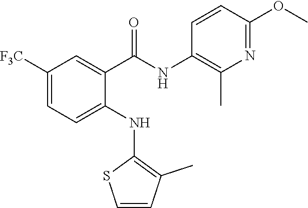 [SMILES: COc1ccc(NC(=O)c2cc(C(F)(F)F)ccc2Nc2sccc2C)c(C)n1] | MS (m/z) 420.0 (M - H) | 6- meth- oxy-2- methyl pyridin- 3- amine | 2-((3- methylthiophen- 2-yl)amino)- 5- (trifluoromethyl)- benzoic acid |
| 508 | 4-cyano-2-((4- fluoro-2- methoxyphenyl) amino)-N-(6- methoxypyridin- 3-yl)benzamide | 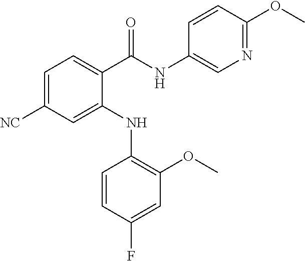 [SMILES: COc1ccc(NC(=O)c2ccc(C#N)cc2Nc2ccc(F)cc2OC)cn1] | MS (m/z) 393.0 (M + H)⁺ | 6- meth- oxypyridin- 3- amine | 4-cyano-2-((4- fluoro-2- methoxyphen- yl)amino)benzoic acid |
| 509 | 2-((4-fluoro-2- methylphenyl)- amino)-N-(6- methoxy-2- methylpyridin-3- yl)-4- (methylsulfonyl)- benzamide | 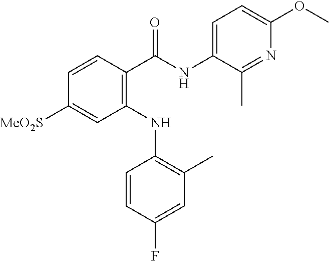 [SMILES: COc1ccc(NC(=O)c2ccc(S(C)(=O)=O)cc2Nc2ccc(F)cc2C)c(C)n1] | MS (m/z) 444.0 (M + H)⁺ | 6- meth- oxy-2- methyl pyridin- 3- amine | 2-((4-fluoro-2- methylphenyl) amino)-4- (methylsulfonyl)- benzoic acid |
| 510 | 5-chloro-2-((2- ethyl-4- fluorophenyl)- amino)-N-(6- methoxy-2- methylpyridin-3- yl)benzamide | 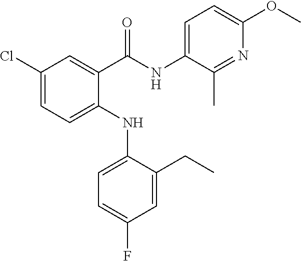 [SMILES: CCc1cc(F)ccc1Nc1ccc(Cl)cc1C(=O)Nc1ccc(OC)nc1C] | MS (m/z) 414.0 (M + H)⁺ | 6- meth- oxy-2- methyl pyridin- 3- amine | 5-chloro-2-((2- ethyl-4- fluorophenyl)- amino)benzoic acid |
| 511 | 5-chloro-2-((4- fluoro-2- isopropylphenyl) amino)-N-(6- methoxy-2,4- dimethylpyridin- 3-yl)benzamide | 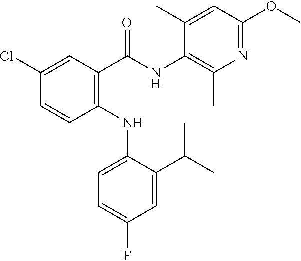 [SMILES: COc1cc(C)c(NC(=O)c2cc(Cl)ccc2Nc2ccc(F)cc2C(C)C)c(C)n1] | MS (m/z) 442.0 (M + H)⁺ | 6- meth- oxy-2,4- dimethy Ipyridin- 3- amine | 5-chloro-2-((4- fluoro-2- isopropylphen- yl)amino)benzoic acid |
| 512 | 2-((4-fluoro-2- methylphenyl)- amino)-N-(6- methoxy-2- methylpyridin-3- yl)-4-(2,2,2- trifluoroethoxy)- benzamide | 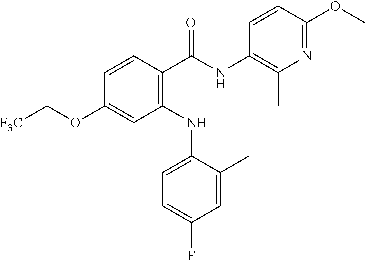 [SMILES: COc1ccc(NC(=O)c2ccc(OCC(F)(F)F)cc2Nc2ccc(F)cc2C)c(C)n1] | MS (m/z) 464.2 (M + H)⁺ | 6- meth- oxy-2- methyl pyridin- 3- amine | 2-((4-fluoro-2- methylphenyl) amino)-4- (2,2,2- trifluoroethoxy)- benzoic acid |
| 513 | N-(2-ethyl-6- methoxypyridin- 3-yl)-2-((4-fluoro- 2- isopropylphenyl) amino)-4- (trifluoromethyl)- benzamide | 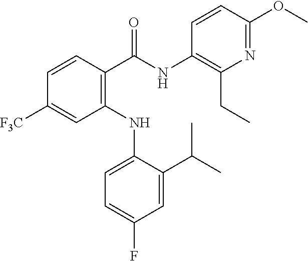 [SMILES: CCc1nc(OC)ccc1NC(=O)c1ccc(C(F)(F)F)cc1Nc1ccc(F)cc1C(C)C] | MS (m/z) 475.8 (M + H)⁺ | 2-ethyl- 6- meth- oxypyridin- 3- amine | 2-((4-fluoro-2- isopropylphen- yl)amino)-4- (trifluoromethyl)- benzoic acid |
| 514 | 2-((4-fluoro-2- methylphenyl)- amino)-N-(5- methoxypyrazin- 2-yl)-4- (trifluoromethyl)- benzamide | 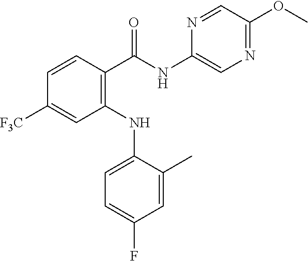 [SMILES: COc1cnc(NC(=O)c2ccc(C(F)(F)F)cc2Nc2ccc(F)cc2C)cn1] | MS (m/z) 421.1 (M)⁺ | 5- meth- oxypyrazi n-2- amine | 2-((4-fluoro-2- methylphenyl) amino)-4- (trifluoromethyl)- benzoic acid |
| 515 | 5-fluoro-2-((4- fluoro-2- methylphenyl)- amino)-N-(6- methoxy-2- methylpyridin-3- yl)-4- methylbenzamide | 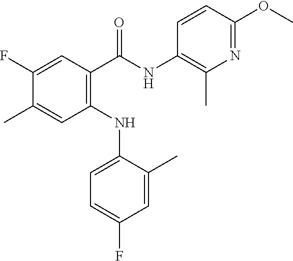 | MS (m/z) 398.1 (M)⁺ | 6- meth- oxy-2- methyl pyridin- 3- amine | 5-fluoro-2-((4- fluoro-2- methylphenyl) amino)-4- methylbenzoic acid |
| 516 | 2-(benzylamino)- N-(6-methoxy-2- methylpyridin-3- yl)-4- (trifluoromethyl)- benzamide | 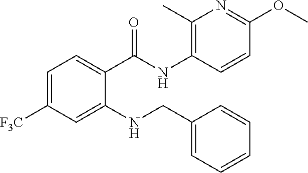 [SMILES: COc1ccc(NC(=O)c2ccc(C(F)(F)F)cc2NCc2ccccc2)c(C)n1] | MS (m/z) 416.2 (M)⁺ | 6- meth- oxy-2- methyl pyridin- 3- amine | 2- (benzylamino)- 4- (trifluoromethyl)- benzoic acid |
| 517 | 2-chloro-6-((4- fluoro-2- methylphenyl)- amino)-N-(6- methoxy-2- methylpyridin-3- yl)benzamide | 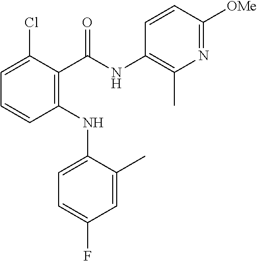 [SMILES: COc1ccc(NC(=O)c2c(Cl)cccc2Nc2ccc(F)cc2C)c(C)n1] | MS (m/z) 400.1 (M)⁺ | 6- meth- oxy-2- methyl pyridin- 3- amine | 2-chloro-6-((4- fluoro-2- methylphenyl) amino)benzoic acid |
| 518 | 5-chloro-2-((4- fluoro-2- methoxyphenyl) amino)-N-(6- methoxy-2- methylpyridin-3- yl)benzamide | 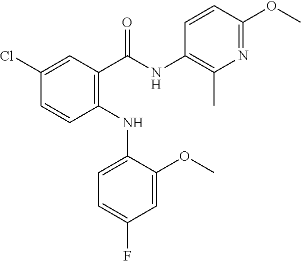 [SMILES: COc1ccc(NC(=O)c2cc(Cl)ccc2Nc2ccc(F)cc2OC)c(C)n1] | MS (m/z) 416.0 (M)⁺ | 6- meth- oxy-2- methyl pyridin- 3- amine | 5-chloro-2-((4- fluoro-2- methoxyphen- yl)amino)benzoic acid |
| 519 | 2-((4-fluoro-2- methylphenyl)- amino)-N-(3- methoxy-1- methyl-1H- pyrazol-5-yl)-4- (trifluoromethyl)- benzamide | 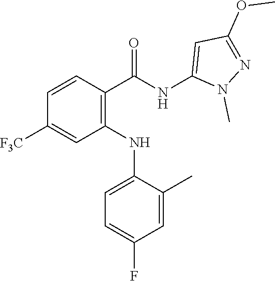 [SMILES: COc1cc(NC(=O)c2ccc(C(F)(F)F)cc2Nc2ccc(F)cc2C)n(C)n1] | MS (m/z) 423.0 (M + H)⁺ | 3- meth- oxy-1- methyl- 1H- pyrazol- 5- amine | 2-((4-fluoro-2- methylphenyl) amino)-4- (trifluoromethyl)- benzoic acid |
| 520 | 4-fluoro-2-((4- fluoro-2- isopropylphenyl) amino)-N-(6- methoxy-2- methylpyridin-3- yl)benzamide | 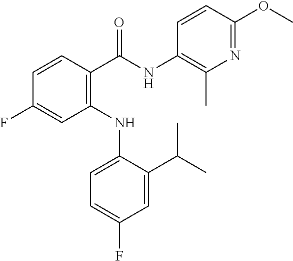 [SMILES: COc1ccc(NC(=O)c2ccc(F)cc2Nc2ccc(F)cc2C(C)C)c(C)n1] | MS (m/z) 412.0 (M + H)⁺ | 6- meth- oxy-2- methyl pyridin- 3- amine | 4-fluoro-2-((4- fluoro-2- isopropylphen- yl)amino)benzoic acid |
| 521 | 2-((2-(tert-butyl)- 4- fluorophenyl)- amino)-5-chloro-N- (6-methoxy-2- methylpyridin-3- yl)benzamide | 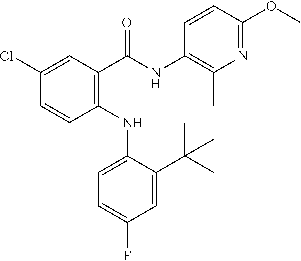 [SMILES: COc1ccc(NC(=O)c2cc(Cl)ccc2Nc2ccc(F)cc2C(C)(C)C)c(C)n1] | MS (m/z) 442.0 (M + H)⁺ | 6- meth- oxy-2- methyl pyridin- 3- amine | 2-((2-(tert- butyl)-4- fluorophenyl)- amino)-5- chloro benzoic acid |
| 522 | 2-((2-methyl-4- (trifluoromethoxy)- phenyl)amino)- N-(3- methylpyridin-4- yl)-5- (trifluoromethyl)- benzamide | 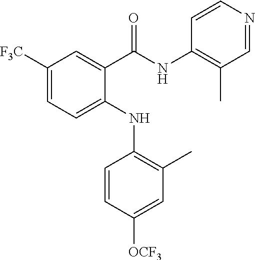 [SMILES: Cc1cnccc1NC(=O)c1cc(C(F)(F)F)ccc1Nc1ccc(OC(F)(F)F)cc1C] | MS (m/z) 469.8 (M + H)⁺ | 3- methyl pyridin- 4- amine | 2-((2-methyl- 4- (trifluorometh- oxy)phenyl)- amino)-5- (trifluoromethyl)- benzoic acid |
| 523 | 2-((4-fluoro-2- methylphenyl)- amino)-N-(2- methoxy-4,6- dimethylpyrimidin- 5-yl)-4- (trifluoromethyl)- benzamide | 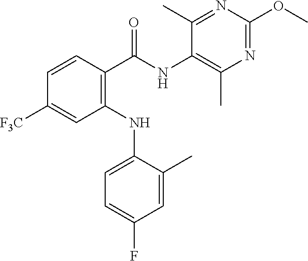 [SMILES: COc1nc(C)c(NC(=O)c2ccc(C(F)(F)F)cc2Nc2ccc(F)cc2C)c(C)n1] | MS (m/z) 449.2 (M + H)⁺ | 2- meth- oxy-4,6- dimethy Ipyrimid in-5- amine | 2-((4-fluoro-2- methylphenyl) amino)-4- (trifluoromethyl)- benzoic acid |
| 524 | N-(6- methoxypyridin- 3-yl)-2-((2- methyl-4- (trifluoromethoxy)- phenyl)amino)- 5- (trifluoromethyl)- benzamide | 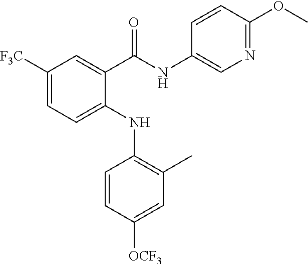 [SMILES: COc1ccc(NC(=O)c2cc(C(F)(F)F)ccc2Nc2ccc(OC(F)(F)F)cc2C)cn1] | MS (m/z) 485.8 (M)⁺ | 6- meth- oxypyridin- 3- amine | 2-((2-methyl- 4- (trifluorometh- oxy)phenyl)- amino)-5- (trifluoromethyl)- benzoic acid |
| 525 | 2-((3,4-difluoro- 2- methylphenyl)- amino)-N-(6- methoxy-2- methylpyridin-3- yl)-5- (trifluoromethyl)- benzamide | 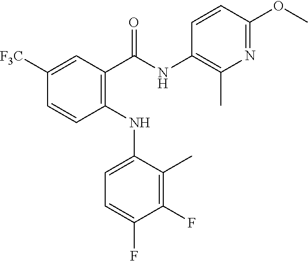 [SMILES: COc1ccc(NC(=O)c2cc(C(F)(F)F)ccc2Nc2ccc(F)c(F)c2C)c(C)n1] | MS (m/z) 452.0 (M + H)⁺ | 6- meth- oxy-2- methyl pyridin- 3- amine | 2-((3,4- difluoro-2- methylphenyl) amino)-5- (trifluoromethyl)- benzoic acid |
| 526 | 5-chloro-N-(4- chloro-6- methoxy-2- methylpyridin-3- yl)-2-((4-fluoro-2- methylphenyl)- amino)benzamide | 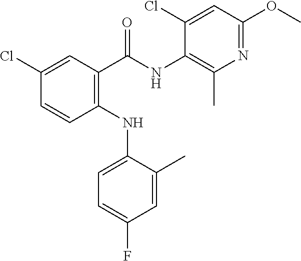 [SMILES: COc1cc(Cl)c(NC(=O)c2cc(Cl)ccc2Nc2ccc(F)cc2C)c(C)n1] | MS (m/z) 434.0 (M + H)⁺ | 4- chloro- 6- meth- oxy-2- methyl pyridin- 3- amine | 5-chloro-2-((4- fluoro-2- methylphenyl) amino)benzoic acid |
| 527 | 2-((4-fluoro-2- methylphenyl)- amino)-N-(6- methoxy-4- methylpyridazin- 3-yl)-4- (trifluoromethyl)- benzamide | 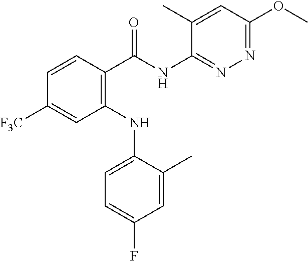 [SMILES: COc1cc(C)c(NC(=O)c2ccc(C(F)(F)F)cc2Nc2ccc(F)cc2C)nn1] | MS (m/z) 434.8 (M)⁺ | 6- meth- oxy-4- methyl pyridazin- 3- amine | 2-((4-fluoro-2- methylphenyl) amino)-4- (trifluoromethyl)- benzoic acid |
| 528 | 4-chloro-5- cyano-2-((4- fluoro-2- methylphenyl)- amino)-N-(6- methoxy-2- methylpyridin-3- yl)benzamide | 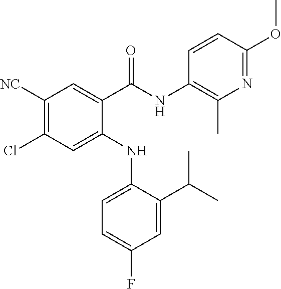 | MS (m/z) 424.8 (M)⁺ | 6- meth- oxy-2- methyl pyridin- 3- amine | 4-chloro-5- cyano-2-((4- fluoro-2- methylphenyl) amino)benzoic acid |
| 529 | 3-chloro-6-((4- fluoro-2- isopropylphenyl) amino)-N-(6- methoxy-2- methylpyridin-3- yl)-2- methylbenzamide | 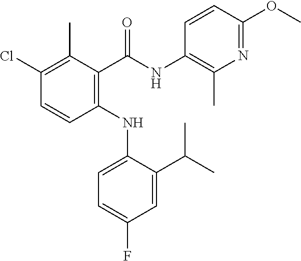 [SMILES: COc1ccc(NC(=O)c2c(Nc3ccc(F)cc3C(C)C)ccc(Cl)c2C)c(C)n1] | MS (m/z) 442.0 (M + H)⁺ | 6- meth- oxy-2- methyl pyridin- 3- amine | 3-chloro-6-((4- fluoro-2- isopropylphen- yl)amino)-2- methylbenzoic acid |
| 530 | 5-chloro-4- fluoro-2-((4- fluoro-2- isopropylphenyl) amino)-N-(6- methoxy-2- methylpyridin-3- yl)benzamide | 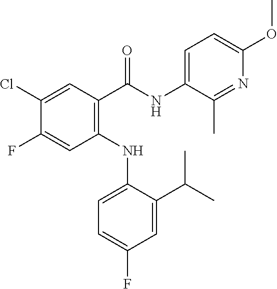 [SMILES: COc1ccc(NC(=O)c2cc(Cl)c(F)cc2Nc2ccc(F)cc2C(C)C)c(C)n1] | MS (m/z) 446.0 (M + H)⁺ | 6- meth- oxy-2- methyl pyridin- 3- amine | 5-chloro-4- fluoro-2-((4- fluoro-2- isopropylphen- yl)amino)benzoic acid |
| 531 | 2-((4-fluoro-2- methylphenyl)- amino)-4- methoxy-N-(6- methoxy-2- methylpyridin-3- yl)benzamide | 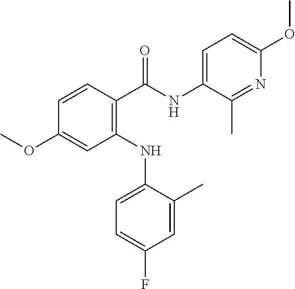 | MS (m/z) 396.2 (M + H)⁺ | 6- meth- oxy-2- methyl pyridin- 3- amine | 2-((4-fluoro-2- methylphenyl) amino)-4- methoxybenzoic acid |
| 532 | 2-((4-fluoro-2- methylphenyl)- amino)-5- methoxy-N-(6- methoxy-2- methylpyridin-3- yl)-4- (trifluoromethyl)- benzamide | 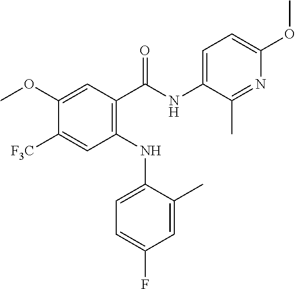 | MS (m/z) 464.1 (M + H)⁺ | 6- meth- oxy-2- methyl pyridin- 3- amine | 2-((4-fluoro-2- methylphenyl) amino)-5- methoxy-4- (trifluoromethyl)- benzoic acid |
| 533 | 5-chloro-2-((4- fluoro-2- methylphenyl)- amino)-N-(6- methoxy-2- methylpyridin-3- yl)nicotinamide | 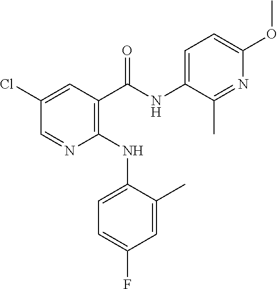 [SMILES: COc1ccc(NC(=O)c2cc(Cl)cnc2Nc2ccc(F)cc2C)c(C)n1] | MS (m/z) 401.2 (M + H)⁺ | 6- meth- oxy-2- methyl pyridin- 3- amine | 5-chloro-2-((4- fluoro-2- methylphenyl) amino)nicotinic acid |
| 534 | 2-((4-fluoro-2- isopropylphenyl) amino)-N-(6- methoxy-2- methylpyridin-3- yl)-4- (trifluoromethoxy)- benzamide | 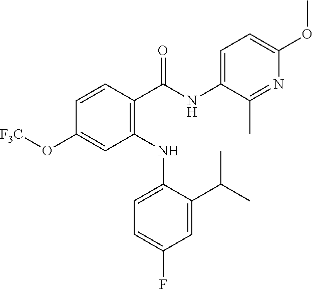 [SMILES: COc1ccc(NC(=O)c2ccc(OC(F)(F)F)cc2Nc2ccc(F)cc2C(C)C)c(C)n1] | MS (m/z) 478.2 (M + H)⁺ | 6- meth- oxy-2- methyl pyridin- 3- amine | 2-((4-fluoro-2- isopropylphen- yl)amino)-4- (trifluorometh- oxy)benzoic acid |
| 535 | 5-chloro-4- fluoro-2-((4- fluoro-2- methylphenyl)- amino)-N-(6- methoxy-2- methylpyridin-3- yl)benzamide | 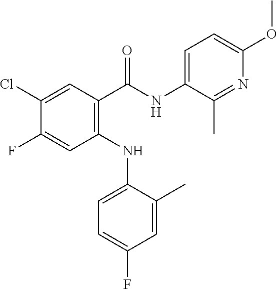 [SMILES: COc1ccc(NC(=O)c2cc(Cl)c(F)cc2Nc2ccc(F)cc2C)c(C)n1] | MS (m/z) 417.8 (M)⁺ | 6- meth- oxy-2- methyl pyridin- 3- amine | 5-chloro-4- fluoro-2-((4- fluoro-2- methylphenyl) amino)benzoic acid |
| 536 | 5-chloro-N-(2- ethyl-6- methoxypyridin- 3-yl)-2-((4-fluoro- 2- methylphenyl)- amino)benzamide | 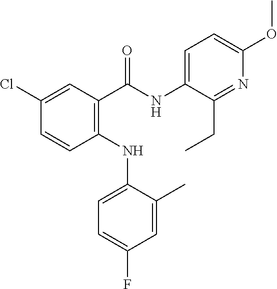 [SMILES: CCc1nc(OC)ccc1NC(=O)c1cc(Cl)ccc1Nc1ccc(F)cc1C] | MS (m/z) 414.0 (M + H)⁺ | 2-ethyl- 6- meth- oxypyridin- 3- amine | 5-chloro-2-((4- fluoro-2- methylphenyl) amino)benzoic acid |
| 537 | 4-fluoro-2-((4- fluoro-2- methylphenyl)- amino)-N-(6- methoxy-2- methylpyridin-3- yl)-5- (trifluoromethyl)- benzamide | 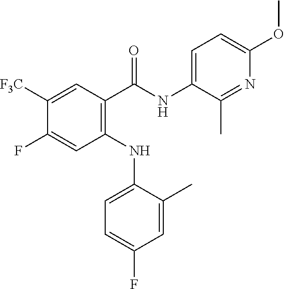 [SMILES: COc1ccc(NC(=O)c2cc(C(F)(F)F)c(F)cc2Nc2ccc(F)cc2C)c(C)n1] | MS (m/z) 452.0 (M + H)⁺ | 6- meth- oxy-2- methyl pyridin- 3- amine | 4-fluoro-2-((4- fluoro-2- methylphenyl) amino)-5- (trifluoromethyl)- benzoic acid |
| 538 | 2-fluoro-6-((4- fluoro-2- methylphenyl)- amino)-N-(6- methoxy-2- methylpyridin-3- yl)-3- (trifluoromethyl)- benzamide | 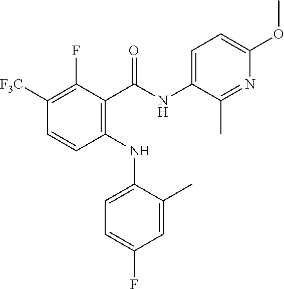 [SMILES: COc1ccc(NC(=O)c2c(Nc3ccc(F)cc3C)ccc(C(F)(F)F)c2F)c(C)n1] | MS (m/z) 452.0 (M + H)⁺ | 6- meth- oxy-2- methyl pyridin- 3- amine | 2-fluoro-6-((4- fluoro-2- methylphenyl) amino)-3- (trifluoromethyl)- benzoic acid |
| 539 | 5-fluoro-2-((4- fluoro-2- methylphenyl)- amino)-N-(6- methoxy-2- methylpyridin-3- yl)-4- (trifluoromethyl)- benzamide | 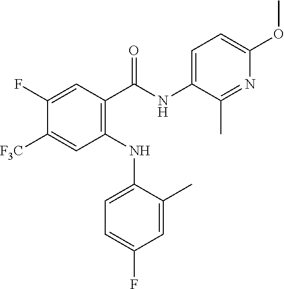 [SMILES: COc1ccc(NC(=O)c2cc(F)c(C(F)(F)F)cc2Nc2ccc(F)cc2C)c(C)n1] | MS (m/z) 452.0 (M + H)⁺ | 6- meth- oxy-2- methyl pyridin- 3- amine | 5-fluoro-2-((4- fluoro-2- methylphenyl) amino)-4- (trifluoromethyl)- benzoic acid |
| 540 | 5-bromo-4- fluoro-2-((4- fluoro-2- methylphenyl)- amino)-N-(6- methoxy-2- methylpyridin-3- yl)benzamide | 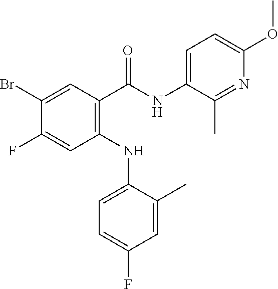 [SMILES: COc1ccc(NC(=O)c2cc(Br)c(F)cc2Nc2ccc(F)cc2C)c(C)n1] | MS (m/z) 462.0 (M + H)^{+/464.0} ^{(M+3H)+} | 6- meth- oxy-2- methyl pyridin- 3- amine | 5-bromo-4- fluoro-2-((4- fluoro-2- methylphenyl) amino)benzoic acid |
| 541 | 5-bromo-2-((4- fluoro-2- methylphenyl)- amino)-N-(6- methoxy-2- methylpyridin-3- yl)-4- (trifluoromethoxy)- benzamide | 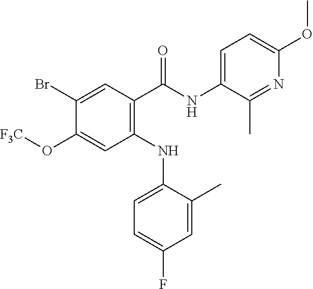 [SMILES: COc1ccc(NC(=O)c2cc(Br)c(OC(F)(F)F)cc2Nc2ccc(F)cc2C)c(C)n1] | MS (m/z) 528.0(M + H)^{+/530.0} ^{(M+3H)+} | 6- meth- oxy-2- methyl pyridin- 3- amine | 5-bromo-2- ((4-fluoro-2- methylphenyl) amino)-4- (trifluorometh- oxy)benzoic acid |
| 542 | 2-((4,5-difluoro- 2- methylphenyl)- amino)-N-(6- methoxy-2- methylpyridin-3- yl)-5- (trifluoromethyl)- benzamide | 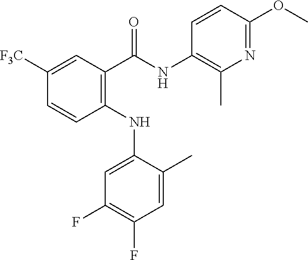 [SMILES: COc1ccc(NC(=O)c2cc(C(F)(F)F)ccc2Nc2cc(F)c(F)cc2C)c(C)n1] | MS (m/z) 452.0 (M + H)⁺ | 6- meth- oxy-2- methyl pyridin- 3- amine | 2-((4,5- difluoro-2- methylphenyl) amino)-5- (trifluoromethyl)- benzoic acid |
| 543 | 3-chloro-6-((4- fluoro-2- methylphenyl)- amino)-N-(6- methoxy-2- methylpyridin-3- yl)-2- methylbenzamide | 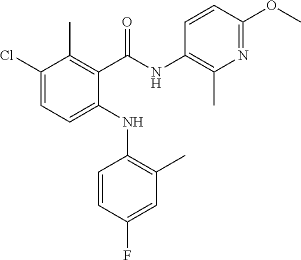 [SMILES: COc1ccc(NC(=O)c2c(Nc3ccc(F)cc3C)ccc(Cl)c2C)c(C)n1] | MS (m/z) 414.2 (M + H)⁺ | 6- meth- oxy-2- methyl pyridin- 3- amine | 3-chloro-6-((4- fluoro-2- methylphenyl) amino)-2- methylbenzoic acid |
| 544 | 2-((2-(tert-butyl)- 4- fluorophenyl)- amino)-N-(6- methoxy-2- methylpyridin-3- yl)-4- (trifluoromethyl)- benzamide | 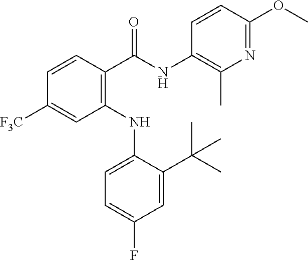 [SMILES: COc1ccc(NC(=O)c2ccc(C(F)(F)F)cc2Nc2ccc(F)cc2C(C)(C)C)c(C)n1] | MS (m/z) 476.1 (M + H)⁺ | 6- meth- oxy-2- methyl pyridin- 3- amine | 2-((2-(tert- butyl)-4- fluorophenyl)- amino)-4- (trifluoromethyl)- benzoic acid |
| 545 | 2-((3,5-difluoro- 2- methylphenyl)- amino)-N-(6- methoxy-2- methylpyridin-3- yl)-5- (trifluoromethyl)- benzamide | 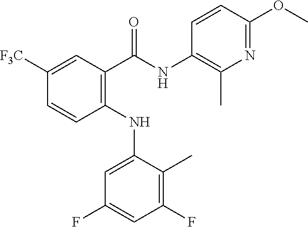 [SMILES: COc1ccc(NC(=O)c2cc(C(F)(F)F)ccc2Nc2cc(F)cc(F)c2C)c(C)n1] | MS (m/z) 452.2 (M + H)⁺ | 6- meth- oxy-2- methyl pyridin- 3- amine | 2-((3,5- difluoro-2- methylphenyl) amino)-5- (trifluoromethyl)- benzoic acid |
| 546 | 2-((4-fluoro-2- isopropylphenyl) amino)-6- methoxy-N-(6- methoxy-2- methylpyridin-3- yl)benzamide | 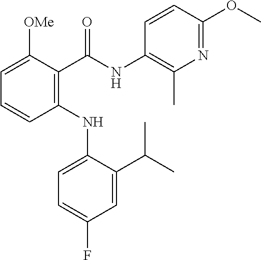 [SMILES: COc1ccc(NC(=O)c2c(Nc3ccc(F)cc3C(C)C)cccc2OC)c(C)n1] | MS (m/z) 423.8 (M + H)⁺ | 6- meth- oxy-2- methyl pyridin- 3- amine | 2-((4-fluoro-2- isopropylphen- yl)amino)-6- methoxybenzoic acid |
| 547 | 2-((4-fluoro-2- methylphenyl)- amino)-N-(2- methoxy-4- methylpyrimidin- 5-yl)-4- (trifluoromethyl)- benzamide | 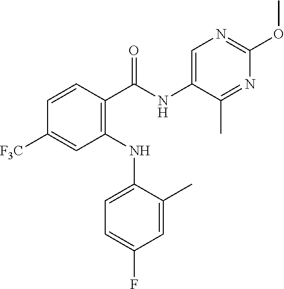 [SMILES: COc1ncc(NC(=O)c2ccc(C(F)(F)F)cc2Nc2ccc(F)cc2C)c(C)n1] | MS (m/z) 435.1 (M + H)⁺ | 2- meth- oxy-4- methyl pyrimidin- 5- amine | 2-((4-fluoro-2- methylphenyl) amino)-4- (trifluoromethyl)- benzoic acid |
| 548 | 2-((2-ethyl-4- fluorophenyl)- amino)-5-fluoro-N- (6-methoxy-2- methylpyridin-3- yl)benzamide | 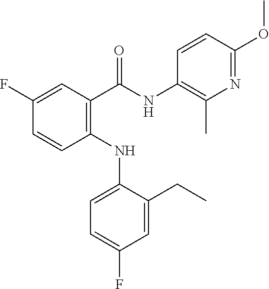 [SMILES: CCc1cc(F)ccc1Nc1ccc(F)cc1C(=O)Nc1ccc(OC)nc1C] | MS (m/z) 398.1 (M + H)⁺ | 6- meth- oxy-2- methyl pyridin- 3- amine | 2-((2-ethyl-4- fluorophenyl)- amino)-5- fluorobenzoic acid |
| 549 | 5-chloro-2-((4- cyano-2- methylphenyl)- amino)-N-(6- methoxy-2- methylpyridin-3- yl)benzamide | 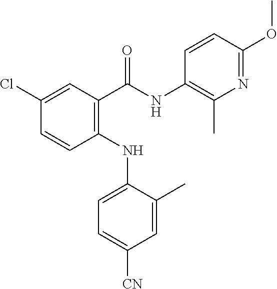 [SMILES: COc1ccc(NC(=O)c2cc(Cl)ccc2Nc2ccc(C#N)cc2C)c(C)n1] | MS (m/z) 407.2 (M + H)⁺ | 6- meth- oxy-2- methyl pyridin- 3- amine | 5-chloro-2-((4- cyano-2- methylphenyl) amino)benzoic acid |
| 550 | N-(6- methoxypyridin- 3-yl)-2-((2- methyl-4- (trifluoromethoxy)- phenyl)amino)- 5- (trifluoromethyl)- nicotinamide | 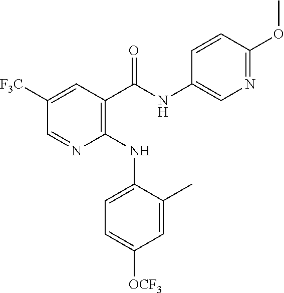 [SMILES: COc1ccc(NC(=O)c2cc(C(F)(F)F)cnc2Nc2ccc(OC(F)(F)F)cc2C)cn1] | MS (m/z) 487.0 (M + H)⁺ | 6- meth- oxypyridin- 3- amine | 2-((2-methyl- 4- (trifluorometh- oxy)phenyl)- amino)-5- (trifluoromethyl)- nicotinic acid |
| 551 | 2-((2,4-difluoro- 6- methylphenyl)- amino)-N-(6- methoxy-2- methylpyridin-3- yl)-5- (trifluoromethyl)- benzamide | 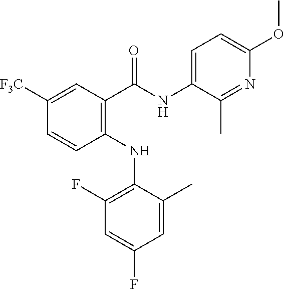 [SMILES: COc1ccc(NC(=O)c2cc(C(F)(F)F)ccc2Nc2c(C)cc(F)cc2F)c(C)n1] | MS (m/z) 452.2 (M + H)⁺ | 6- meth- oxy-2- methyl pyridin- 3- amine | 2-((2,4- difluoro-6- methylphenyl) amino)-5- (trifluoromethyl)- benzoic acid |
| 552 | 6- (difluoromethyl)- 2-((4-fluoro-2- isopropylphenyl) amino)-N-(6- methoxy-2- methylpyridin-3- yl)nicotinamide | 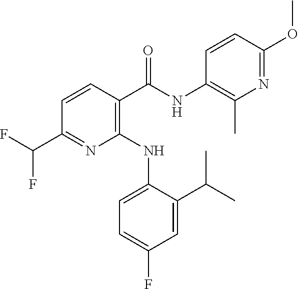 [SMILES: COc1ccc(NC(=O)c2ccc(C(F)F)nc2Nc2ccc(F)cc2C(C)C)c(C)n1] | MS (m/z) 445.0 (M + H)⁺ | 6- meth- oxy-2- methyl pyridin- 3- amine | 6- (difluoromethyl)- -2-((4-fluoro- 2- isopropylphen- yl)amino)nicotinic acid |
| 553 | 2-((4-cyano-2- methylphenyl)- amino)-N-(6- methoxy-2- methylpyridin-3- yl)-5- (trifluoromethoxy)- benzamide | 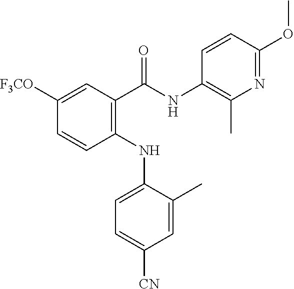 [SMILES: COc1ccc(NC(=O)c2cc(OC(F)(F)F)ccc2Nc2ccc(C#N)cc2C)c(C)n1] | MS (m/z) 457.2 (M + H)⁺ | 6- meth- oxy-2- methyl pyridin- 3- amine | 2-((4-cyano-2- methylphenyl) amino)-5- (trifluorometh- oxy)benzoic acid |
| 554 | 3-chloro-2-((4- fluoro-2- methylphenyl)- amino)-N-(6- methoxy-2- methylpyridin-3- yl)benzamide | 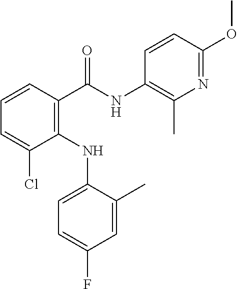 [SMILES: COc1ccc(NC(=O)c2cccc(Cl)c2Nc2ccc(F)cc2C)c(C)n1] | MS (m/z) 400.0 (M + H)⁺ | 6- meth- oxy-2- methyl pyridin- 3- amine | 3-chloro-2-((4- fluoro-2- methylphenyl) amino)benzoic acid |
| 555 | 2-((4-methoxy-2- methylphenyl)- amino)-N-(6- methoxy-2- methylpyridin-3- yl)-5- (trifluoromethyl)- benzamide | 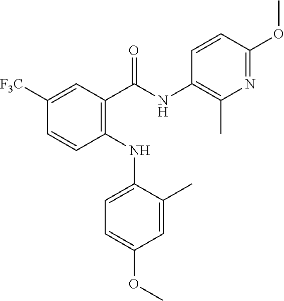 [SMILES: COc1ccc(Nc2ccc(C(F)(F)F)cc2C(=O)Nc2ccc(OC)nc2C)c(C)c1] | MS (m/z) 446.1 (M + H)⁺ | 6- meth- oxy-2- methyl pyridin- 3- amine | 2-((4- methoxy-2- methylphenyl) amino)-5- (trifluoromethyl)- benzoic acid |
| 556 | 5-chloro-2-((3- cyano-2- methylphenyl)- amino)-N-(6- methoxy-2- methylpyridin-3- yl)benzamide | 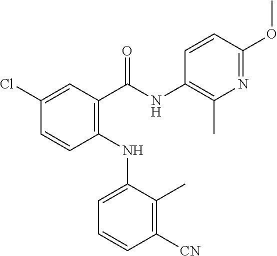 [SMILES: COc1ccc(NC(=O)c2cc(Cl)ccc2Nc2cccc(C#N)c2C)c(C)n1] | MS (m/z) 407.0 (M + H)⁺ | 6- meth- oxy-2- methyl pyridin- 3- amine | 5-chloro-2-((3- cyano-2- methylphenyl) amino)benzoic acid |
| 557 | 5-fluoro-2-((4- fluoro-2- methylphenyl)- amino)-N-(6- methoxy-2- methylpyridin-3- yl)-4- (trifluoromethoxyl)- aenzamide | 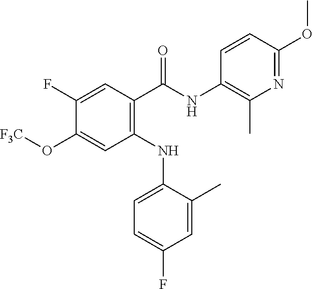 [SMILES: COc1ccc(NC(=O)c2cc(F)c(OC(F)(F)F)cc2Nc2ccc(F)cc2C)c(C)n1] | MS (m/z) 468.0 (M + H)⁺ | 6- meth- oxy-2- methyl pyridin- 3- amine | 5-fluoro-2-((4- fluoro-2- methylphenyl) amino)-4- (trifluorometh- oxy)benzoic acid |
| 558 | 2-((5-cyano-2- methylphenyl)- amino)-N-(6- methoxy-2- methylpyridin-3- yl)-5- (trifluoromethyl)- benzamide | 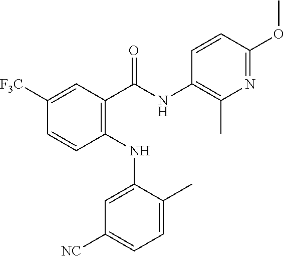 [SMILES: COc1ccc(NC(=O)c2cc(C(F)(F)F)ccc2Nc2cc(C#N)ccc2C)c(C)n1] | MS (m/z) 441.2 (M + H)⁺ | 6- meth- oxy-2- methyl pyridin- 3- amine | 2-((5-cyano-2- methylphenyl) amino)-5- (trifluoromethyl)- benzoic acid |
| 559 | 2- (difluoromethyl)- 6-((4-fluoro-2- methylphenyl)- amino)-N-(6- methoxy-2- methylpyridin-3- yl)benzamide | 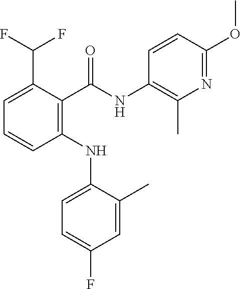 [SMILES: COc1ccc(NC(=O)c2c(Nc3ccc(F)cc3C)cccc2C(F)F)c(C)n1] | MS (m/z) 416.0 (M + H)⁺ | 6- meth- oxy-2- methyl pyridin- 3- amine | 2- (difluoromethyl)- -6-((4-fluoro- 2- methylphenyl) amino)benzoic acid |
| 560 | 2-((4-fluoro-2- methylphenyl)- amino)-N-(5- fluoro-6- methoxy-2- methylpyridin-3- yl)-5- (trifluoromethyl)- benzamide | 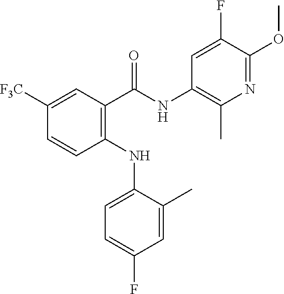 [SMILES: COc1nc(C)c(NC(=O)c2cc(C(F)(F)F)ccc2Nc2ccc(F)cc2C)cc1F] | MS (m/z) 451.8 (M)⁺ | 5- fluoro- 6- meth- oxy-2- methyl pyridin- 3- amine | 2-((4-fluoro-2- methylphenyl) amino)-5- (trifluoromethyl)- benzoic acid |
| 561 | methyl 4-(5- chloro-2-((4- fluoro-2- methylphenyl)- amino)benzamido)- furan-2- carboxylate | 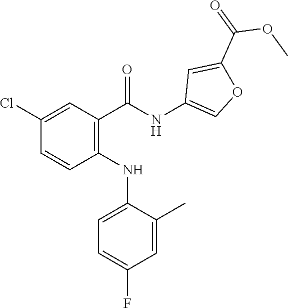 [SMILES: COC(=O)c1cc(NC(=O)c2cc(Cl)ccc2Nc2ccc(F)cc2C)co1] | MS (m/z) 403.0 (M + H)⁺ | methyl 4- amino- furan-2- carboxy late | 5-chloro-2-((4- fluoro-2- methylphenyl) amino)benzoic acid |
| 562 | 3-chloro-2- fluoro-6-((4- fluoro-2- methylphenyl)- amino)-N-(2- methoxypyridin- 4-yl)benzamide | 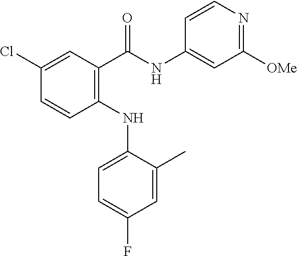 | MS (m/z) 404.0 (M + H)⁺ | 2- meth- oxypyridin- 4- amine | 3-chloro-2- fluoro-6-((4- fluoro-2- methylphenyl) amino)benzoic acid |
| 563 | 4,5-difluoro-2- ((4-fluoro-2- methoxyphenyl) amino)-N-(6- methoxy-2- methylpyridin-3- yl)benzamide | 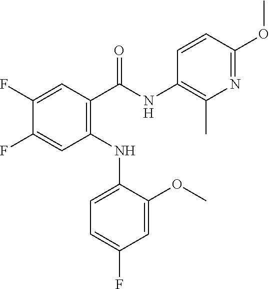 [SMILES: COc1ccc(NC(=O)c2cc(F)c(F)cc2Nc2ccc(F)cc2OC)c(C)n1] | MS (m/z) 417.8 (M + H)⁺ | 6- meth- oxy-2- methyl pyridin- 3- amine | 4,5-difluoro-2- ((4-fluoro-2- methoxyphen- yl)amino)benzoic acid |
| 564 | 5-chloro-N-(6- methoxy-2- methylpyridin-3- yl)-2-((2- methoxy-4- methylphenyl)- amino)benzamide | 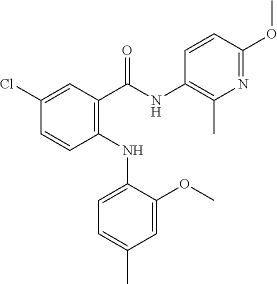 [SMILES: COc1ccc(NC(=O)c2cc(Cl)ccc2Nc2ccc(C)cc2OC)c(C)n1] | MS (m/z) 412.0 (M + H)⁺ | 6- meth- oxy-2- methyl pyridin- 3- amine | 5-chloro-2-((2- methoxy-4- methylphenyl) amino)benzoic acid |
| 565 | 2-((2-fluoro-6- methylphenyl)- amino)-N-(6- methoxy-2- methylpyridin-3- yl)-5- (trifluoromethyl)- benzamide | 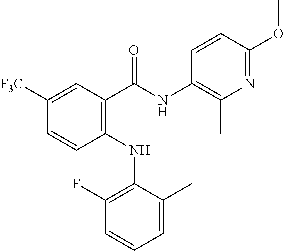 [SMILES: COc1ccc(NC(=O)c2cc(C(F)(F)F)ccc2Nc2c(C)cccc2F)c(C)n1] | MS (m/z) 434.0 (M + H)⁺ | 6- meth- oxy-2- methyl pyridin- 3- amine | 2-((2-fluoro-6- methylphenyl) amino)-5- (trifluoromethyl)- benzoic acid |
| 566 | N-(3- bromophenyl)-2- ((4-fluoro-2- methylphenyl)- amino)-4- (trifluoromethyl)- benzamide | 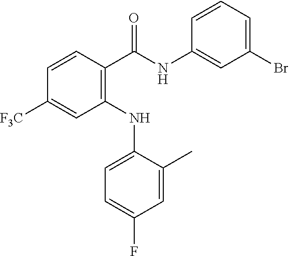 [SMILES: Cc1cc(F)ccc1Nc1cc(C(F)(F)F)ccc1C(=O)Nc1cccc(Br)c1] | MS (m/z) 467(M + H)⁺/469 (M + 3H)+ | 3- bromo- aniline | 2-((4-fluoro-2- methylphenyl) amino)-4- (trifluoromethyl)- benzoic acid |
| 567 | 2-fluoro-N-(6- methoxy-2- methylpyridin-3- yl)-5- (trifluoromethyl)- tenzamide | 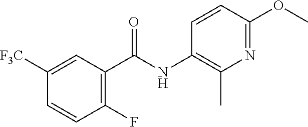 [SMILES: COc1ccc(NC(=O)c2cc(C(F)(F)F)ccc2F)c(C)n1] | MS (m/z) 329.2 (M + H)⁺ | 6- meth- oxy-2- methyl pyridin- 3- amine | 2-fluoro-5- (trifluoromethyl)- benzoic acid |
| 568 | 4- (difluoromethyl)- 2-((4-fluoro-2- isopropylphenyl) amino)-N-(6- methoxy-2- methylpyridin-3- yl)benzamide) | 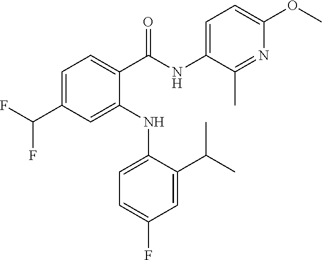 [SMILES: COc1ccc(NC(=O)c2ccc(C(F)F)cc2Nc2ccc(F)cc2C(C)C)c(C)n1] | MS (m/z) 444.1 (M + H)⁺ | 6- meth- oxy-2- methyl pyridin- 3- amine | 4- (difluoromethyl)- -2-((4-fluoro- 2- isopropylphen- yl)amino)benzoic acid |
| 569 | 4- (difluoromethyl)- 2-((4-fluoro-2- methylphenyl)- amino)-N-(6- methoxy-2- methylpyridin-3- yl)benzamide | 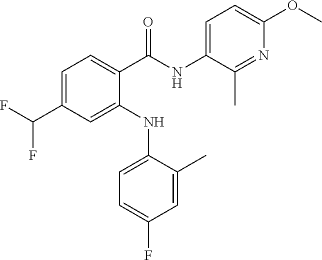 [SMILES: COc1ccc(NC(=O)c2ccc(C(F)F)cc2Nc2ccc(F)cc2C)c(C)n1] | MS (m/z) 416.0 (M + H)⁺ | 6- meth- oxy-2- methyl pyridin- 3- amine | 4- (difluoromethyl)- 2-((4-fluoro- 2- methylphenyl) amino)benzoic acid |
| 570 | 2- (bicyclo[1.1.1]pen- tan-1-ylamino)- N-(6-methoxy-2- methylpyridin-3- yl)-4- (trifluoromethyl)- benzamide | 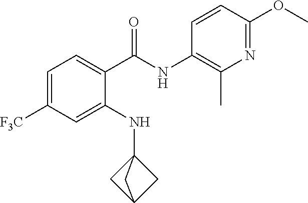 [SMILES: COc1ccc(NC(=O)c2ccc(C(F)(F)F)cc2NC23CC(C2)C3)c(C)n1] | MS (m/z) 392.2 (M + H)⁺ | 6- meth- oxy-2- methyl pyridin- 3- amine | 2- (bicyclo[1.1.1] pentan-1- ylamino)-4- (trifluoromethyl)- benzoic acid |
| 571 | 5-chloro-N-(2- ethyl-6- methoxypyridin- 3-yl)-2-((4-fluoro- 2- isopropylphenyl) amino)benzamide | 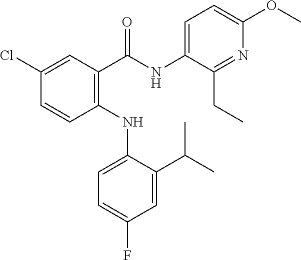 [SMILES: CCc1nc(OC)ccc1NC(=O)c1cc(Cl)ccc1Nc1ccc(F)cc1C(C)C] | MS (m/z) 442.0 (M + H)⁺ | 2-ethyl- 6- meth- oxypyridin- 3- amine | 5-chloro-2-((4- fluoro-2- isopropylphen- yl)amino)benzoic acid |
| 572 | 2-((3-cyano-4- fluoro-2- methylphenyl)- amino)-N-(6- methoxy-2- methylpyridin-3- yl)-5- (trifluoromethyl)- benzamide | 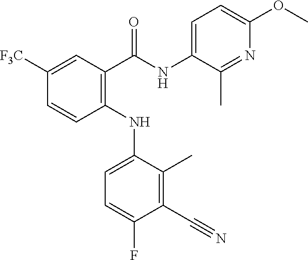 [SMILES: COc1ccc(NC(=O)c2cc(C(F)(F)F)ccc2Nc2ccc(F)c(C#N)c2C)c(C)n1] | MS (m/z) 459.0 (M + H)⁺ | 6- meth- oxy-2- methyl pyridin- 3- amine | 2-((3-cyano-4- fluoro-2- methylphenyl) amino)-5- (trifluoromethyl)- benzoic acid |
| 573 | 5-fluoro-2-((4- fluoro-2- isopropylphenyl) amino)-N-(6- methoxy-2- methylpyridin-3- yl)nicotinamide | 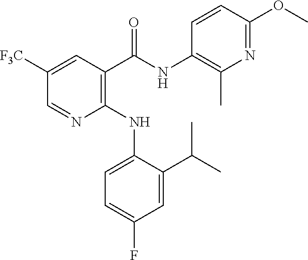 | MS (m/z) 413.1 (M + H)⁺ | 6- meth- oxy-2- methyl pyridin- 3- amine | 5-fluoro-2-((4- fluoro-2- isopropylphen- yl)amino)nicotinic acid |
| 574 | 5-fluoro-2-((4- fluoro-2- methylphenyl)- amino)-N-(6- methoxy-2- methylpyridin-3- yl)benzamide | 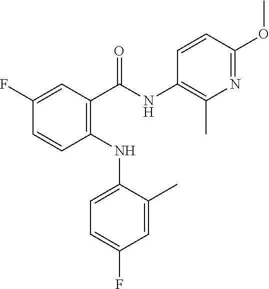 [SMILES: COc1ccc(NC(=O)c2cc(F)ccc2Nc2ccc(F)cc2C)c(C)n1] | MS (m/z) 384.1 (M + H)⁺ | 6- meth- oxy-2- methyl pyridin- 3- amine | 5-fluoro-2-((4- fluoro-2- methylphenyl) amino)benzoic acid |
| 575 | 2-((4-fluoro-2- methylphenyl)- amino)-N-(2- methoxy-3- methylpyridin-4- yl)-5- (trifluoromethyl)- benzamide | 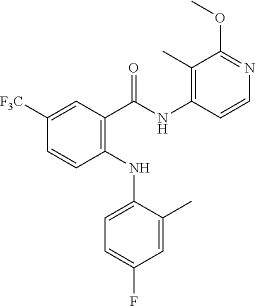 [SMILES: COc1nccc(NC(=O)c2cc(C(F)(F)F)ccc2Nc2ccc(F)cc2C)c1C] | MS (m/z) 434.2 (M + H)⁺ | 2- meth- oxy-3- methyl pyridin- 4- amine | 2-((4-fluoro-2- methylphenyl) amino)-5- (trifluoromethyl)- benzoic acid |
| 576 | N-(6-chloro-4- methylpyridin-3- yl)-2-((4-fluoro-2- methylphenyl)- amino)-5- (trifluoromethyl)- benzamide | 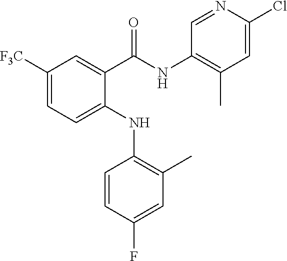 [SMILES: Cc1cc(Cl)ncc1NC(=O)c1cc(C(F)(F)F)ccc1Nc1ccc(F)cc1C] | MS (m/z) 437.8 (M)⁺ | 6- chloro- 4- methyl pyridin- 3- amine | 2-((4-fluoro-2- methylphenyl) amino)-5- (trifluoromethyl)- benzoic acid |
| 577 | N-(2,6- dioxopiperidin-3- yl)-2-((4-fluoro-2- methylphenyl)- amino)-5- (trifluoromethyl)- benzamide | 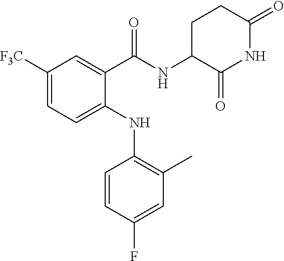 [SMILES: Cc1cc(F)ccc1Nc1ccc(C(F)(F)F)cc1C(=O)NC1CCC(=O)NC1=O] | MS (m/z) 424.3 (M)⁺ | 3-amino- piperidine- 2,6- dione hydro- chloride | 2-((4-fluoro-2- methylphenyl) amino)-5- (trifluoromethyl)- benzoic acid |

### Intermediate 578

### 4-Cyano-2-((4-fluoro-2-methylphenyl)amino)-N-(6-methoxypyridin-3-yl)benzamide

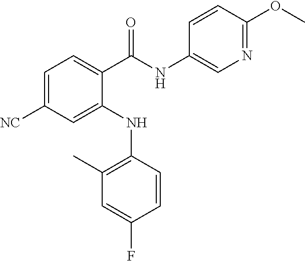 [SMILES: COc1ccc(NC(=O)c2ccc(C#N)cc2Nc2ccc(F)cc2C)cn1]

To a solution of 4-fluoro-2-methylaniline (1.385 g, 11.07 mmol) in THF (3 mL) was added LiHMDS (14.76 mL, 14.76 mmol) at −78° C. and the reaction was stirred at the same temperature for 20 minutes. 4-cyano-2-fluoro-N-(6-methoxypyridin-3-yl)benzamide (2.0016 g, 7.38 mmol) was added and the reaction mixture was stirred at 28° C. for 16 hours. The reaction mixture was quenched with a mixture of ice water and MeOH (1:1) and then concentrated in vacuo. To the residue was added water (20 mL) and the reaction was extracted with DCM (2×50 mL). The combined organic extracts were washed with brine, dried over Na₂SO₄, filtered and concentrated. The residue was purified by column chromatography (Isolera, 50 g SNAP column, 0-25% EtOAc/petroleum ether) to give the title compound as a yellow solid (608 mg, 1.373 mmol, 18.61% yield). MS (m/z) 377.0 (M+H)⁺.

Intermediates 579-580 were prepared from the indicated aryl fluoride and aniline by methods analogous to those described for Intermediate 578.

| Int. | Name | Structure | Characterization | Aryl Fluoride | Aniline |
|---|---|---|---|---|---|
| 579 | 3-chloro-2-((4- fluoro-2- methylphenyl)- amino)-N-(6- methoxypyridin-3- yl)-4- (trifluoromethyl)- benzamide | 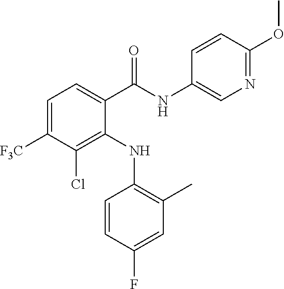 [SMILES: COc1ccc(NC(=O)c2ccc(C(F)(F)F)c(Cl)c2Nc2ccc(F)cc2C)cn1] | MS (m/z) 454.0 (M + H)⁺ | 3-chloro-2- fluoro-N-(6- methoxypyridin- 3-yl)-4- (trifluoromethyl)- benzamide | 4-fluoro- 2- methyl- aniline |
| 580 | 2-((2-bromo-4- fluorophenyl)- amino)-N-(6- methoxy- 2-methylpyridin-3- yl)-5- (trifluoromethyl)- benzamide | 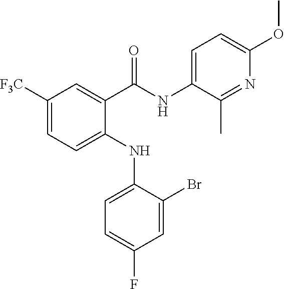 [SMILES: COc1ccc(NC(=O)c2cc(C(F)(F)F)ccc2Nc2ccc(F)cc2Br)c(C)n1] | MS (m/z) 498.0 (M + H)⁺ | 2-fluoro-N-(6- methoxy-2- methylpyridin- 3-yl)-5- (trifluoromethyl)- benzamide | 2-bromo- 4- fluoroaniline |

### Intermediate 581

### N-(2,6-Dimethoxypyrimidin-4-yl)-2-((4-fluoro-2-methylphenyl)amino)-4-(trifluoromethyl)benzamide

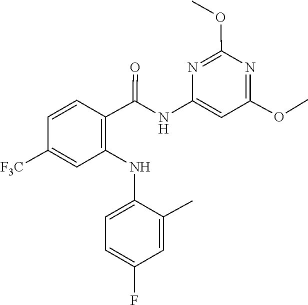 [SMILES: COc1cc(NC(=O)c2ccc(C(F)(F)F)cc2Nc2ccc(F)cc2C)nc(OC)n1]

A 30 mL microwave vial, fitted with a magnetic stir bar was charged with ethyl 2-((4-fluoro-2-methylphenyl)amino)-4-(trifluoromethyl)benzoate (600 mg, 1.758 mmol), 2,6-dimethoxypyrimidin-4-amine (273 mg, 1.758 mmol) and THF (5 mL) at RT. DABAL-Me3 (451 mg, 1.758 mmol) was added portionwise to the reaction mixture at 0° C. The reaction vessel was sealed and heated in an Anton Parr at 130° C. for 1 hr. The reaction mixture was quenched with ice water (20 mL) dropwise and extracted with EtOAc (2×25 mL). The combined organic extracts were washed with brine (25 mL), dried over Na₂SO₄ and concentrated. The crude product was purified by column chromatography (Isolera, 100 g SNAP column, 0-30% EtOAc/petroleum ether over 30 min) to give the title product as a yellow solid (610 mg, 1.183 mmol, 67.3% yield). MS (m/z) 451.0 (M+H)⁺.

### Intermediate 582

### Cis-rac-N-(6-methoxy-2-methylpyridin-3-yl)-2-(((3R,4R)-3-methyltetrahydro-2H-pyran-4-yl)amino)-4-(trifluoromethyl)benzamide

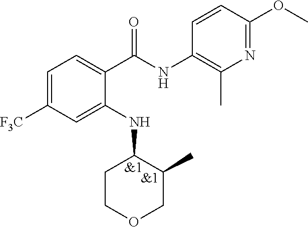 [SMILES: COc1ccc(NC(=O)c2ccc(C(F)(F)F)cc2N[C@@H]2CCOC[C@@H]2C)c(C)n1]

Cis-rac-2-(((3R,4R)-3-methyltetrahydro-2H-pyran-4-yl)amino)-4-(trifluoromethyl)benzoic acid (0.367 g, 1.210 mmol) was dissolved in thionyl chloride (0.883 ml, 12.10 mmol) in a 20 ml vial and was stirred at 60° C. for two hours. In separate flask, 6-methoxy-2-methylpyridin-3-amine (0.159 g, 1.150 mmol) was dissolved in DCM (3.03 ml). Pyridine (0.098 ml, 1.210 mmol) was added followed by the addition of the above acid chloride in DCM (9.08 ml). The reaction was stirred at room temperature for 20 hours. The reaction was concentrated under reduced pressure and the residue was purified by column chromatography eluting with a gradient of 0-10% EtOAc/DCM to provide the title compound (390 mg, 0.921 mmol, 76% yield). MS (m/z) 424 (M+H)⁺.

Intermediates 583-587 were prepared from the indicated carboxylic acid and amine by methods analogous to those described for Intermediate 582

| | | | | | Carboxylic |
|---|---|---|---|---|---|
| Int. | Name | Structure | Characterization | Amine | Acid |
| 583 | N-(2-bromo-6- methoxypyridin- 3-yl)-2-((4-fluoro- 2- methylphenyl)- amino)-5- (trifluoromethyl)- benzamide | 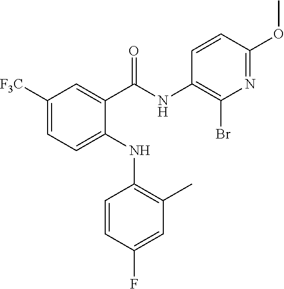 [SMILES: COc1ccc(NC(=O)c2cc(C(F)(F)F)ccc2Nc2ccc(F)cc2C)c(Br)n1] | MS (m/z) 498 (M + H)⁺/500 (M + 3H)+ | 2- bromo- 6- methoxy pyridin- 3-amine | 2-((4-fluoro-2- methylphenyl) amino)-5- (trifluoromethyl)- benzoic acid |
| 584 | trans-rac-N-(6- methoxy-2- methylpyridin-3- yl)-2-(((3R,4S)- 3- methyltetrahydro- 2H-pyran-4- yl)amino)-4- (trifluoromethyl)- benzamide | 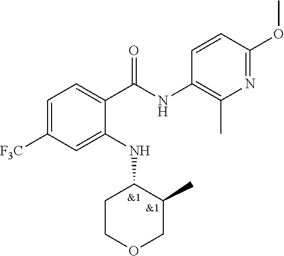 [SMILES: COc1ccc(NC(=O)c2ccc(C(F)(F)F)cc2N[C@H]2CCOC[C@@H]2C)c(C)n1] | MS (m/z) 424 (M + H)⁺ | 6- methoxy- 2- methyl pyridin-3- amine | Trans-rac-2- (((3R,4S)-3- methyltetra- hydro-2H-pyran- 4-yl)amino)-4- (trifluoro- methyl)- benzoic acid |
| 585 | N-(4-chloro-2- methoxy- pyrimidin- 5-yl)-2-((4- fluoro-2- methylphenyl)- amino)-5- (trifluoromethyl)- benzamide | 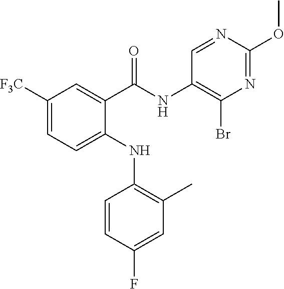 | MS (m/z) 455.3 (M + H)⁺/457 (M + H)⁺ | 4- chloro- 2- methoxy pyrimidin- 5- amine | 2-((4-fluoro-2- methylphenyl) amino)-5- (trifluoro- methyl)- benzoic acid |
| 586 | 6-chloro-5- fluoro-2-((4- fluoro-2- methylphenyl)- amino)-N-(6- methoxy-2- methylpyridin-3- yl)nicotinamide | 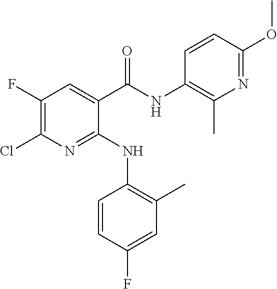 [SMILES: COc1ccc(NC(=O)c2cc(F)c(Cl)nc2Nc2ccc(F)cc2C)c(C)n1] | MS (m/z) 419 (M + H)⁺ | 6- methoxy- 2- methyl pyridin-3- amine | 6-chloro-5- fluoro-2-((4- fluoro-2- methylphenyl) amino)nicotinic acid |
| 587 | methyl 3-(5- chloro-2-((4- fluoro-2- methylphenyl)- amino)benz- amido)- furan-2- carboxylate | 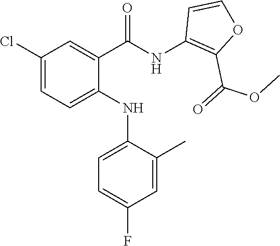 [SMILES: COC(=O)c1occc1NC(=O)c1cc(Cl)ccc1Nc1ccc(F)cc1C] | MS (m/z) 403.0 (M + H)⁺ | methyl 3- aminofur- an-2- carboxy- late | 5-chloro-2-((4- fluoro-2- methylphenyl) amino)benzoic acid |

### Intermediate 588

### 2-((4-Fluoro-2-methylphenyl)amino)-N-(3-methylpyridazin-4-yl)-5-(trifluoromethyl)benzamide

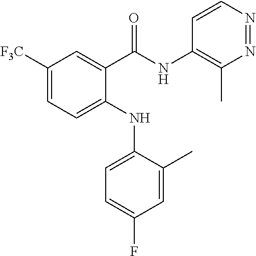 [SMILES: Cc1cc(F)ccc1Nc1ccc(C(F)(F)F)cc1C(=O)Nc1ccnnc1C]

To a suspension of 2-((4-fluoro-2-methylphenyl)amino)-5-(trifluoromethyl)benzoic acid (0.115 g, 0.367 mmol) and 3-methylpyridazin-4-amine, Hydrochloride (0.0996 g, 0.684 mmol) in DCM (4 ml) was added DIEA (0.192 ml, 1.101 mmol) followed by T3P® (50% in EtOAc) (0.328 ml, 0.551 mmol). Reaction was stirred overnight at RT. The reaction was concentrated with nitrogen at 40° C. The residue was purified by column chromatography (silica (12 g) running from 100% heptane to 100% EtOAc) to give the title compound as a yellow oil (103.5 mg, 0.256 mmol, 69.7% yield). MS (m/z) 405.3 (M+H)⁺.

### Intermediate 589

### 1-(4-Fluoro-2-methylphenyl)-3-(6-methoxypyridin-3-yl)-7-(trifluoromethyl)quinazoline-2,4(1H,3H)-dione

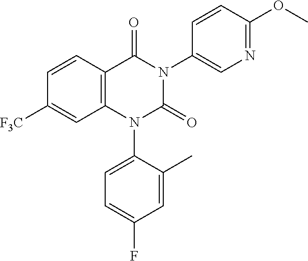 [SMILES: COc1ccc(-n2c(=O)c3ccc(C(F)(F)F)cc3n(-c3ccc(F)cc3C)c2=O)cn1]

To a solution of 2-((4-fluoro-2-methylphenyl)amino)-N-(6-methoxypyridin-3-yl)-4-(trifluoromethyl)benzamide (165 mg, 0.393 mmol) in THF (3.00 mL) were added CDI (159 mg, 0.984 mmol) and DBU (0.148 mL, 0.984 mmol). The reaction was heated to 60° C. and stirred for 2 h. The reaction mixture was cooled to RT and concentrated under reduced pressure. The residue was purified by column chromatography (Isco, 24 g RediSep Rf Gold high performance flash columns, 0-30% EtOAc/heptane over 30 mins) to give the title compound as a colorless oil (158 mg, 0.355 mmol, 90% yield). MS (m/z) 446.3 (M+H)⁺.

### Intermediate 590

### N-(4-((tert-Butyldimethylsilyl)oxy)-2-methylphenyl)-2-((4-fluoro-2-methylphenyl)amino)-5-(trifluoromethyl)benzamide

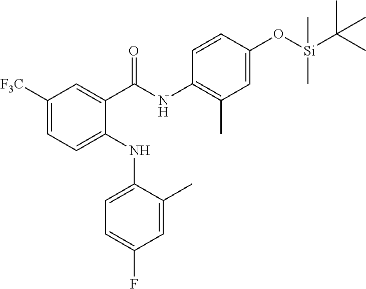 [SMILES: Cc1cc(O[Si](C)(C)C(C)(C)C)ccc1NC(=O)c1cc(C(F)(F)F)ccc1Nc1ccc(F)cc1C]

A mixture of 2-((4-fluoro-2-methylphenyl)amino)-N-(4-hydroxy-2-methylphenyl)-5-(trifluoromethyl)benzamide (100 mg, 0.239 mmol), TBDMS-Cl (108 mg, 0.717 mmol) and imidazole (48.8 mg, 0.717 mmol) in DCM (2390 μl) was stirred for 18 h at RT. Water (5 mL) was added and the reaction was extracted with DCM. The organic layer was dried over MgSO₄ and concentrated. The residue was purified via Isco CombiFlash Rf (24 g Si₂O column, 0%-10% EtOAc/heptane over 15 min) to give the title compound as a white solid (42 mg, 0.079 mmol, 33.0%). MS (m/z) 533.33 (M+H)⁺.

### Intermediate 591

### 1-(4-Fluoro-2-methylphenyl)-3-(6-methoxy-2-methylpyridin-3-yl)-7-(trifluoromethyl)-2,3-dihydroquinazolin-4(1H)-one

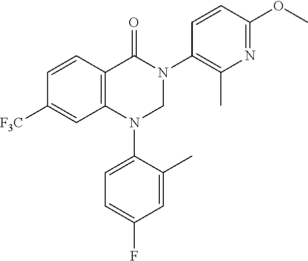 [SMILES: COc1ccc(N2CN(c3ccc(F)cc3C)c3cc(C(F)(F)F)ccc3C2=O)c(C)n1]

Diiodomethane (0.363 mL, 4.50 mmol) was added dropwise to a stirring solution of 2-((4-fluoro-2-methylphenyl)amino)-N-(6-methoxy-2-methylpyridin-3-yl)-4-(trifluoromethyl)benzamide (650 mg, 1.500 mmol) and Cs₂CO₃ (1955 mg, 6.00 mmol) in acetonitrile (25 mL) at 0° C. under N₂. After stirring at 80° C. for 16 hours, the reaction mixture was cooled to RT and filtered through a Celite pad and the pad was washed with EtOAc (3×45 mL). The filtrate was concentrated under reduced pressure and the resultant orange liquid was purified by column chromatography (Biotage, 50 g SNAP column, 0-30% EtOAc/petroleum ether over 45 minutes) to give the title compound as an orange solid (600 mg, 1.118 mmol, 74.5% yield). MS (m/z) 446.0 (M+H)⁺.

Intermediates 592-755 were prepared from the indicated amide by methods analogous to those described for Intermediate 591.

| Int. | Name | Structure | Characterization | Amide |
|---|---|---|---|---|
| 592 | 1-cyclohexyl-3-(6- methoxypyridin-3- yl)-7- (trifluoromethyl)-2,3- dihydroquinazolin- 4(1H)-one | 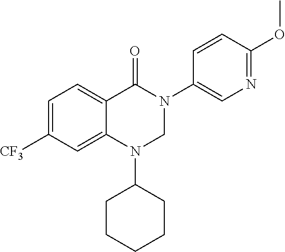 [SMILES: COc1ccc(N2CN(C3CCCCC3)c3cc(C(F)(F)F)ccc3C2=O)cn1] | MS (m/z) 405.9 (M + H)⁺ | 2- (cyclohexylamino)- N-(6- methoxypyridin- 3-yl)-4- (trifluoromethyl) benzamide |
| 593 | 1-(4-fluoro-2,6- dimethylphenyl)-3- (6-methoxypyridin-3- yl)-7- (trifluoromethyl)-2,3- dihydroquinazolin- 4(1H)-one | 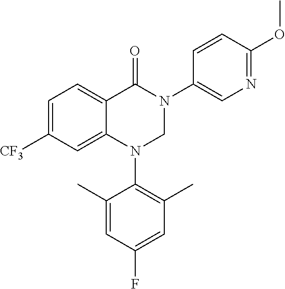 [SMILES: COc1ccc(N2CN(c3c(C)cc(F)cc3C)c3cc(C(F)(F)F)ccc3C2=O)cn1] | MS (m/z) 446.0 (M + H)⁺ | 2-((4-fluoro-2,6- dimethylphenyl) amino)-N-(6- methoxypyridin- 3-yl)-4- (trifluoromethyl) benzamide |
| 594 | 1-(2-chloro-4- fluorophenyl)-3-(6- methoxypyridin-3- yl)-7- (trifluoromethyl)-2,3- dihydroquinazolin- 4(1H)-one | 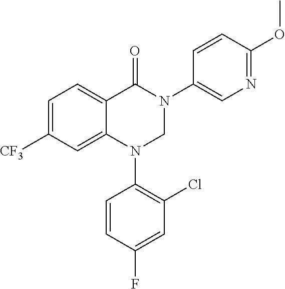 [SMILES: COc1ccc(N2CN(c3ccc(F)cc3Cl)c3cc(C(F)(F)F)ccc3C2=O)cn1] | MS (m/z) 452.0 (M + H)⁺ | 2-((2-chloro-4- fluorophenyl)ami- no)-N-(6- methoxypyridin- 3-yl)-4- (trifluoromethyl) benzamide |
| 595 | 1-(4-fluoro-2-(2- methoxyethoxy)phen- yl)-3-(6- methoxypyridin-3- yl)-7- (trifluoromethyl)-2,3- dihydroquinazolin- 4(1H)-one | 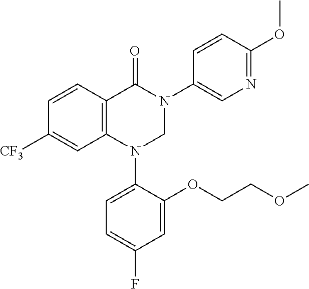 [SMILES: COCCOc1cc(F)ccc1N1CN(c2ccc(OC)nc2)C(=O)c2ccc(C(F)(F)F)cc21] | MS (m/z) 492.0 (M + H)⁺ | 2-((4-fluoro-2- (2- methoxyethoxy) phenyl)amino)- N-(6- methoxypyridin- 3-yl)-4- (trifluoromethyl) benzamide |
| 596 | 1-(2,4- difluorophenyl)-3-(6- methoxypyridin-3- yl)-7- (trifluoromethyl)-2,3- dihydroquinazolin- 4(1H)-one | 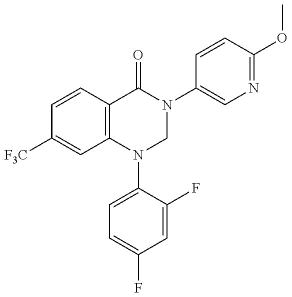 [SMILES: COc1ccc(N2CN(c3ccc(F)cc3F)c3cc(C(F)(F)F)ccc3C2=O)cn1] | MS (m/z) 436.0 (M + H)⁺ | 2-((2,4- difluorophenyl)a- mino)-N-(6- methoxypyridin- 3-yl)-4- (trifluoromethyl) benzamide |
| 597 | 1-(2-ethyl-4- fluorophenyl)-3-(6- methoxypyridin-3- yl)-7- (trifluoromethyl)-2,3- dihydroquinazolin- 4(1H)-one | 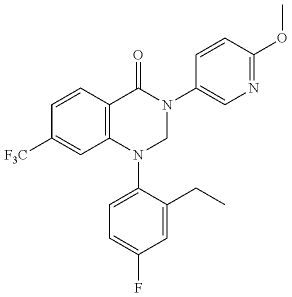 [SMILES: CCc1cc(F)ccc1N1CN(c2ccc(OC)nc2)C(=O)c2ccc(C(F)(F)F)cc21] | MS (m/z) 446.0 (M + H)⁺ | 2-((2-ethyl-4- fluroophenyl)ami- no)-N-(6- methoxypyridin- 3-yl)-4- (trifluoromethyl) benzamide |
| 598 | 8-chloro-1-(4-fluoro- 2-methylphenyl)-3- (6-methoxypyridin-3- yl)-7- (trifluoromethyl)-2,3- dihydroquinazolin- 4(1H)-one | 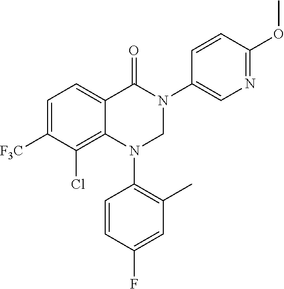 [SMILES: COc1ccc(N2CN(c3ccc(F)cc3C)c3c(ccc(C(F)(F)F)c3Cl)C2=O)cn1] | MS (m/z) 466.0 (M + H)⁺ | 3-chloro-2-((4- fluoro-2- methylphenyl)a- mino)-N-(6- methoxypyridin- 3-yl)-4- (trifluoromethyl) benzamide |
| 599 | 3-(6-methoxy-2- methylpyridin-3-yl)- 1-(2-methylpyridin-3- yl)-7- (trifluoromethyl)-2,3- dihydroquinazolin- 4(1H)-one | 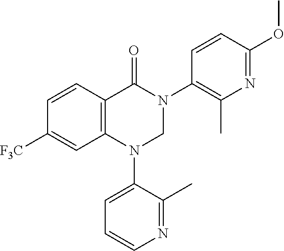 [SMILES: COc1ccc(N2CN(c3cccnc3C)c3cc(C(F)(F)F)ccc3C2=O)c(C)n1] | MS (m/z) 429.0 (M + H)⁺ | N-(6-methoxy- 2-methylpyridin- 3-yl)-2-((2- methylpyridin-3- yl)amino)-4- (trifluoromethyl) benzamide |
| 600 | 1-(4-fluoro-2- isopropylphenyl)-3- (6-methoxy-2- methylpyridin-3-yl)- 7-(trifluoromethyl)- 2,3- dihydroquinazolin- 4(1H)-one | 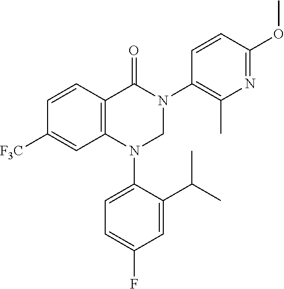 [SMILES: COc1ccc(N2CN(c3ccc(F)cc3C(C)C)c3cc(C(F)(F)F)ccc3C2=O)c(C)n1] | MS (m/z) 474.0 (M + H)⁺ | 2-((4-fluoro-2- isopropylphenyl) amino)-N-(6- methoxy-2- methylpyridin-3- yl)-4- (trifluoromethyl) benzamide |
| 601 | 6-chloro-1-(4-fluoro- 2-methylphenyl)-3- (6-methoxy-2- methylpyridin-3-yl)- 2,3- dihydroquinazolin- 4(1H)-one | 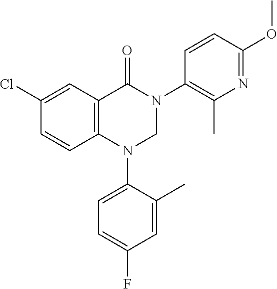 [SMILES: COc1ccc(N2CN(c3ccc(F)cc3C)c3ccc(Cl)cc3C2=O)c(C)n1] | MS (m/z) 412.0 (M + H)⁺ | 5-chloro-2-((4- fluoro-2- methylphenyl)a- mino)-N-(6- methoxy-2- methylpyridin-3- yl)benzamide |
| 602 | 1-(4-fluorophenyl)-3- (6-methoxy-2- methylpyridin-3-yl)- 2,3- dihydroquinazolin- 4(1H)-one | 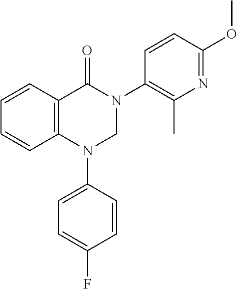 [SMILES: COc1ccc(N2CN(c3ccc(F)cc3)c3ccccc3C2=O)c(C)n1] | MS (m/z) 364.0 (M + H)⁺ | 2-((4- fluorophenyl)ami- no)-N-(6- methoxy-2- methylpyridin-3- yl)benzamide |
| 603 | 3-(6-methoxy-2- methylpyridin-3-yl)- 1-(o-tolyl)-2,3- dihydroquinazolin- 4(1H)-one | 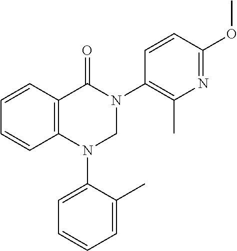 [SMILES: COc1ccc(N2CN(c3ccccc3C)c3ccccc3C2=O)c(C)n1] | MS (m/z) 360.0 (M + H)⁺ | N-(6-methoxy- 2-methylpyridin- 3-yl)-2-(o- tolylamino)benz- amide |
| 604 | 1-(4-fluoro-2- methylphenyl)-3-(6- methoxy-2- mehtylpyridin-3-yl)- 6-methyl-2,3- dihydroquinazolin- 4(1H)-one | 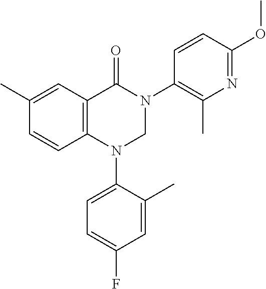 | MS (m/z) 392.2 (M + H)⁺ | 2-((4-fluoro-2- methylphenyl)a- mino)-N-(6- methoxy-2- methylpyridin-3- yl)-5- methylbenzamide |
| 605 | 1-(4-fluoro-2- methylphenyl)-3-(6- methoxy-2- methylpyridin-3-yl)- 2,3- dihydroquinazolin- 4(1H)-one | 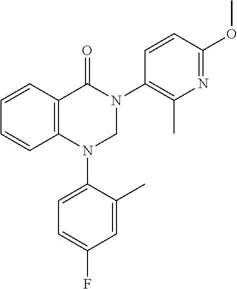 [SMILES: COc1ccc(N2CN(c3ccc(F)cc3C)c3ccccc3C2=O)c(C)n1] | MS (m/z) 378.2 (M + H)⁺ | 2-((4-fluoro-2- methylphenyl)a- mino)-N-(6- methoxy-2- methylpyridin-3- yl)benzamide |
| 606 | 1-(4-fluoro-2- methylphenyl)-3-(2- methoxypyrimidin-5- yl)-7- (trifluoromethyl)-2,3- dihydroquinazolin- 4(1H)-one | 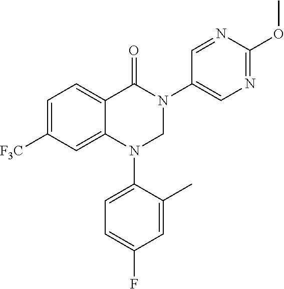 [SMILES: COc1ncc(N2CN(c3ccc(F)cc3C)c3cc(C(F)(F)F)ccc3C2=O)cn1] | MS (m/z) 433.3 (M + H)⁺ | 2-((4-fluoro-2- methylphenyl)a- mino)-N-(2- methoxypyrimidin- 5-yl)-4- (trifluoromethyl) benzamide |
| 607 | 1-(4-bromo-2- methylphenyl)-3-(6- methoxypyridin-3- yl)-7- (trifluoromethyl)-2,3- dihydroquinazolin- 4(1H)-one | 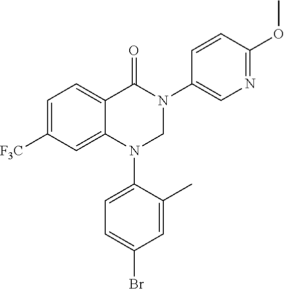 [SMILES: COc1ccc(N2CN(c3ccc(Br)cc3C)c3cc(C(F)(F)F)ccc3C2=O)cn1] | MS (m/z) 492.1 (M + H)⁺ | 2-((4-bromo-2- methylphenyl)a- mino)-N-(6- methoxypyridin- 3-yl)-4- (trifluoromethyl) benzamide |
| 608 | 1-(4-fluorophenyl)-3- (6-methoxy-2- methylpyridin-3-yl)- 7-(trifluoromethyl)- 2,3- dihydroquinazolin- 4(1H)-one | 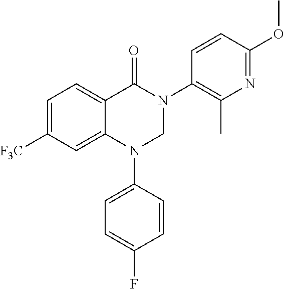 [SMILES: COc1ccc(N2CN(c3ccc(F)cc3)c3cc(C(F)(F)F)ccc3C2=O)c(C)n1] | MS (m/z) 432.3 (M + H)⁺ | 2-((4- fluorophenyl)ami- no)-N-(6- methoxy-2- methylpyridin-3- yl)-4- (trifluoromethyl) benzamide |
| 609 | 1-(4-fluoro-2- methylphenyl)-3-(6- methoxy-2- methylpyridin-3-yl)- 7-(trifluoromethyl)- 2,3- dihydropyrido[2,3- d]pyrimidin-4(1H)- one | 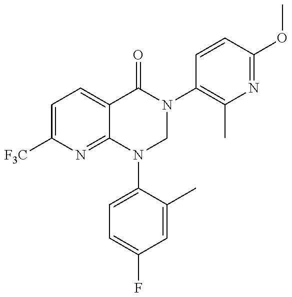 [SMILES: COc1ccc(N2CN(c3ccc(F)cc3C)c3nc(C(F)(F)F)ccc3C2=O)c(C)n1] | MS (m/z) 447.3 (M + H)⁺ | 2-((4-fluoro-2- methylphenyl)a- mino)-N-(6- methoxy-2- methylpyridin-3- yl)-6- (trifluoromethyl) nicotinamide |
| 610 | 3-(2-ethyl-6- methoxypyridin-3- yl)-1-(4-fluoro-2- methylphenyl)-7- (trifluoromethyl)-2,3- dihydroquinazolin- 4(1H)-one | 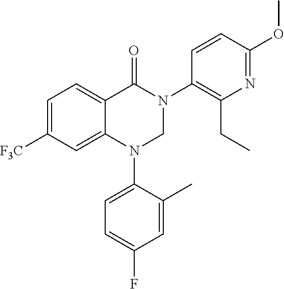 [SMILES: CCc1nc(OC)ccc1N1CN(c2ccc(F)cc2C)c2cc(C(F)(F)F)ccc2C1=O] | MS (m/z) 460.2 (M + H)⁺ | N-(2-ethyl-6- methoxypyridin- 3-yl)-2-((4- fluoro-2- methylphenyl)a- mino)-4- (trifluoromethyl) benzamide |
| 611 | 3-(2-chloro-6- methoxypyridin-3- yl)-1-(4-fluoro-2- methylphenyl)-7- (trifluoromethyl)-2,3- dihydroquinazolin- 4(1H)-one | 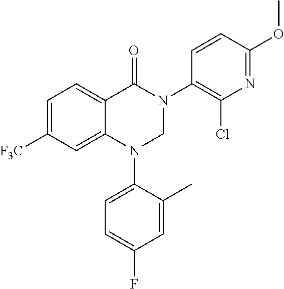 [SMILES: COc1ccc(N2CN(c3ccc(F)cc3C)c3cc(C(F)(F)F)ccc3C2=O)c(Cl)n1] | MS (m/z) 466.2 (M + H)⁺ | N-(2-chloro-6- methoxypyridin- 3-yl)-2-((4- fluoro-2- methylphenyl)a- mino)-4- (trifluoromethyl) benzamide |
| 612 | 7-bromo-1-(4-fluoro- 2-methylphenyl)-3- (6-methoxy-2- methylpyridin-3-yl)- 2,3- dihydroquinazolin- 4(1H)-one | 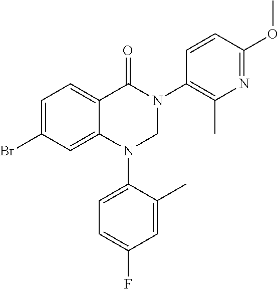 [SMILES: COc1ccc(N2CN(c3ccc(F)cc3C)c3cc(Br)ccc3C2=O)c(C)n1] | MS (m/z) 458.1 (M + 3H)⁺ | 4-bromo-2-((4- fluoro-2- methylphenyl)a- mino)-N-(6- methoxy-2- methylpyridin-3- yl)benzamide |
| 613 | 1-(2,4- difluorophenyl)-3-(6- methoxy-2- methylpyridin-3-yl)- 7-(trifluoromethyl)- 2,3- dihydroquinazolin- 4(1H)-one | 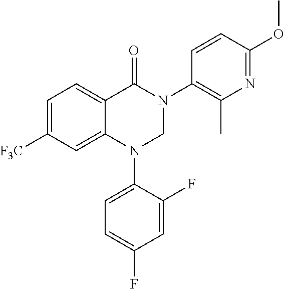 [SMILES: COc1ccc(N2CN(c3ccc(F)cc3F)c3cc(C(F)(F)F)ccc3C2=O)c(C)n1] | MS (m/z) 450.3 (M + H)⁺ | 2-((2,4- difluorophenyl)a- mino)-N-(6- methoxy-2- methlpyridin-3- yl)-4- (trifluoromethyl) benzamide |
| 614 | 1-(4-ethoxyphenyl)- 3-(6-methoxy-2- methylpyridin-3-yl)- 7-(trifluoromethyl)- 2,3- dihydroquinazolin- 4(1H)-one | 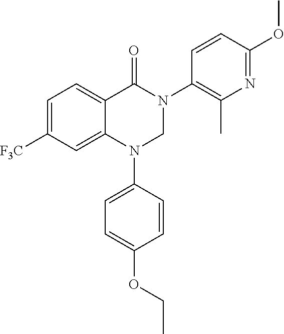 [SMILES: CCOc1ccc(N2CN(c3ccc(OC)nc3C)C(=O)c3ccc(C(F)(F)F)cc32)cc1] | MS (m/z) 458.3 (M + H)⁺ | 2-((4- ethoxyphenyl)a- mino)-N-(6- methoxy-2- methylpyridin-3- yl)-4- (trifluoromethyl) benzamide |
| 615 | 1-(4-fluoro-2- methylphenyl)-3-(6- methoxypyridin-3- yl)-6- (trifluoromethyl)-2,3- dihydroquinazolin- 4(1H)-one | 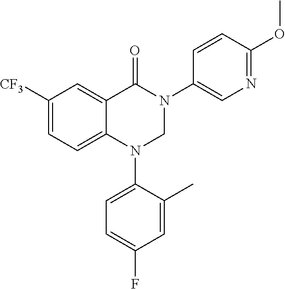 [SMILES: COc1ccc(N2CN(c3ccc(F)cc3C)c3ccc(C(F)(F)F)cc3C2=O)cn1] | MS (m/z) 432.0 (M + H)⁺ | 2-((4-fluoro-2- methylphenyl)a- mino)-N-(6- methoxypridin- 3-yl)-5- (trifluoromethyl) benzamide |
| 616 | 1-(4-fluoro-2- methoxyphenyl)-3- (6-methoxypyridin-3- yl)-7- (trifluoromethyl)-2,3- dihydroquinazolin- 4(1H)-one | 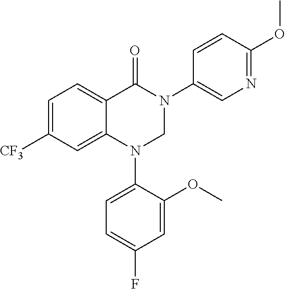 [SMILES: COc1ccc(N2CN(c3ccc(F)cc3OC)c3cc(C(F)(F)F)ccc3C2=O)cn1] | MS (m/z) 448.0 (M + H)⁺ | 2-((4-fluoro-2- methoxyphenyl) amino)-N-(6- methoxypyridin- 3-yl)-4- (trifluoromethyl) benzamide |
| 617 | 1-(4-fluoro-2- methylphenyl)-3-(6- methoxypyridin-3- yl)-5- (trifluoromethyl)-2,3- dihydroquinazolin- 4(1H)-one | 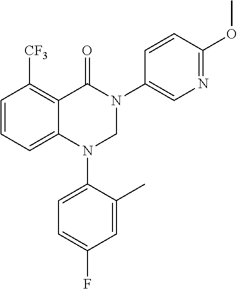 [SMILES: COc1ccc(N2CN(c3ccc(F)cc3C)c3cccc(C(F)(F)F)c3C2=O)cn1] | MS (m/z) 431.9 (M + H)⁺ | 2-((4-fluoro-2- methylphenyl)a- mino)-N-(6- methoxpyridin- 3-yl)-6- (trifluoromethyl) benzamide |
| 618 | 1-(4-fluoro-2- methylphenyl)-3-(6- methoxy-5- methylpyridin-3-yl)- 7-(trifluoromethyl)- 2,3- dihydroquinazolin- 4(1H)-one | 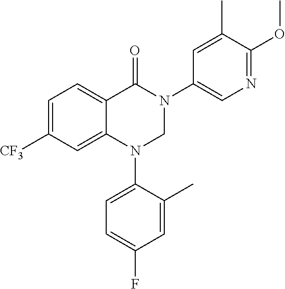 [SMILES: COc1ncc(N2CN(c3ccc(F)cc3C)c3cc(C(F)(F)F)ccc3C2=O)cc1C] | MS (m/z) 445.9 (M + H)⁺ | 2-((4-fluoro-2- methylphenyl)a- mino)-N-(6- methoxy-5- methylpyridin-3- yl)-4- (trifluoromethyl) benzamide |
| 619 | 1-(4-fluoro-2- methylphenyl)-3-(6- methoxypyridazin-3- yl)-7- (trifluoromethyl)-2,3- dihydroquinazolin- 4(1H)-one | 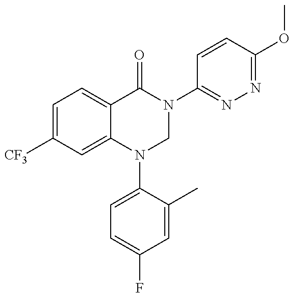 [SMILES: COc1ccc(N2CN(c3ccc(F)cc3C)c3cc(C(F)(F)F)ccc3C2=O)nn1] | MS (m/z) 433.0 (M + H)⁺ | 2-((4-fluoro-2- methylphenyl)a- mino)-N-(6- methoxpyridazin- 3-yl)-4- (trifluoromethyl) benzamide |
| 620 | 1-(4-fluoro-2- methylphenyl)-3-(6- methoxy-4- methylpyridin-3-yl)- 7-(trifluoromethyl)- 2,3- dihydroquinazolin- 4(1H)-one | 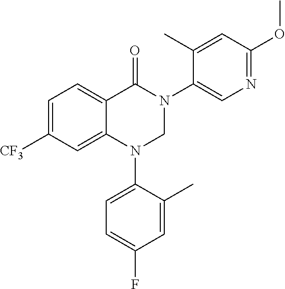 [SMILES: COc1cc(C)c(N2CN(c3ccc(F)cc3C)c3cc(C(F)(F)F)ccc3C2=O)cn1] | MS (m/z) 445.9 (M + H)⁺ | 2-((4-fluoro-2- methylphenyl)a- mino)-N-(6- methoxy-4- methylpyridin-3- yl)-4- (trifluoromethyl) benzamide |
| 621 | 1-(4-fluoro-2- methylphenyl)-3-(6- methoxypyridin-3- yl)-4-oxo-1,2,3,4- tetrahydroquinazoline- 7-carbonitrile | 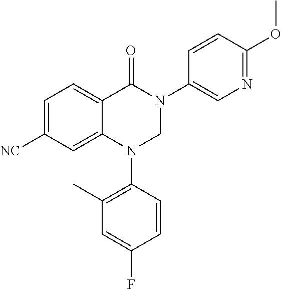 [SMILES: COc1ccc(N2CN(c3ccc(F)cc3C)c3cc(C#N)ccc3C2=O)cn1] | MS (m/z) 389.1 (M + H)⁺ | 4-cyano-2-((4- fluoro-2- methylphenyl)a- mino)-N-(6- methoxypyridin- 3-yl)benzamide |
| 622 | 1-(4-fluoro-2- methoxyphenyl)-3- (6-methoxypyridin-3- yl)-5- (trifluoromethyl)-2,3- dihydroquinazolin- 4(1H)-one | 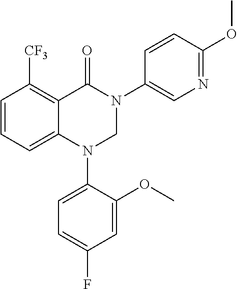 [SMILES: COc1ccc(N2CN(c3ccc(F)cc3OC)c3cccc(C(F)(F)F)c3C2=O)cn1] | MS (m/z) 448.0 (M + H)⁺ | 2-((4-fluoro-2- methoxyphenyl) amino)-N-(6- methoxpyridin- 3-yl)-6- (trifluoromethyl) benzamide |
| 623 | 1-(4-bromo-2- methylphenyl)-3-(6- methoxy-2- methylpyridin-3-yl)- 7-(trifluoromethyl)- 2,3- dihydroquinazolin- 4(1H)-one | 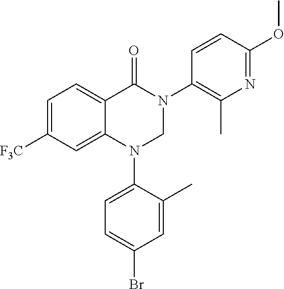 [SMILES: COc1ccc(N2CN(c3ccc(Br)cc3C)c3cc(C(F)(F)F)ccc3C2=O)c(C)n1] | MS (m/z) 508.1 (M + H)⁺ | 2-((4-bromo-2- methylphenyl)a- mino)-N-(6- methoxy-2- methylpyridin-3- yl)-4- (trifluoromethyl) benzamide |
| 624 | 3-(2-bromo-6- methoxypyridin-3- yl)-1-(4-fluoro-2- methylphenyl)-7- (trifluoromethyl)-2,3- dihydroquinazolin- 4(1H)-one | 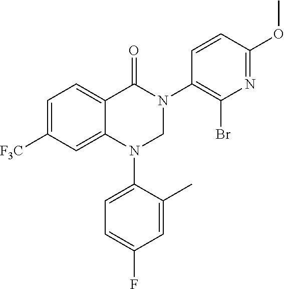 [SMILES: COc1ccc(N2CN(c3ccc(F)cc3C)c3cc(C(F)(F)F)ccc3C2=O)c(Br)n1] | MS (m/z) 512.1 (M + 3H)⁺ | N-(2-bromo-6- methoxypyridin- 3-yl)-2-((4- fluoro-2- methylphenyl)a- mino)-4- (trifluoromethyl) benzamide |
| 625 | 1-(4-fluoro-2- methylphenyl)-3-(2- methoxy-6- methylpyridin-4-yl)- 7-(trifluoromethyl)- 2,3- dihydroquinazolin- 4(1H)-one | 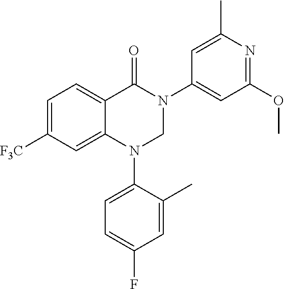 [SMILES: COc1cc(N2CN(c3ccc(F)cc3C)c3cc(C(F)(F)F)ccc3C2=O)cc(C)n1] | MS (m/z) 446.0 (M + H)⁺ | 2-((4-fluoro-2- methylphenyl)a- mino)-N-(2- methoxy-6- methylpyridin-4- yl)-4- (trifluoromethyl) benzamide |
| 626 | 1-(4-fluroo-2- methylphenyl)-3-(2- methoxy-5- methylpyridin-4-yl)- 7-(trifluoromethyl)- 2,3- dihydroquinazolin- 4(1H)-one | 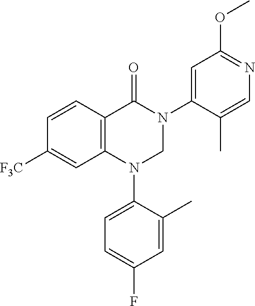 [SMILES: COc1cc(N2CN(c3ccc(F)cc3C)c3cc(C(F)(F)F)ccc3C2=O)c(C)cn1] | MS (m/z) 446.0 (M + H)⁺ | 2-((4-fluoro-2- methylphenyl)a- mino)-N-(2- methoxy-5- methylpyridin-4- yl)-4- (trifluoromethyl) benzamide |
| 627 | 1-(4-fluoro-2- methylphenyl)-3-(2- methoxy-3- methylpyridin-4-yl)- 7-(trifluoromethyl)- 2,3- dihydroquinazolin- 4(1H)-one | 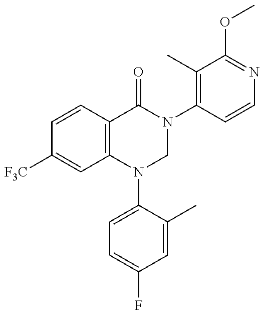 [SMILES: COc1nccc(N2CN(c3ccc(F)cc3C)c3cc(C(F)(F)F)ccc3C2=O)c1C] | MS (m/z) 446.0 (M + H)⁺ | 2-((4-fluoro-2- methylphenyl)a- mino)-N-(2- methoxy-3- methylpyridin-4- yl)-4- (trifluoromethyl) benzamide |
| 628 | 1-(4-fluoro-2- methylphenyl)-3-(6- methoxy-2- methylpyridin-3-yl)- 7-(trifluoromethoxy)- 2,3- dihydroquinazolin- 4(1H)-one | 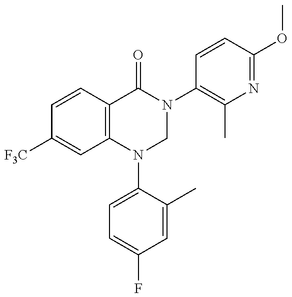 | MS (m/z) 462.0 (M + H)⁺ | 2-((4-fluoro-2- methylphenyl)a- mino)-N-(6- methoxy-2- methylpyridin-3- yl)-4- (trifluoromethoxy) benzamide |
| 629 | 7-chloro-1-(4-fluoro- 2-methylphenyl)-3- (6-methoxy-2- methylpyridin-3-yl)- 2,3- dihydroquinazolin- 4(1H)-one | 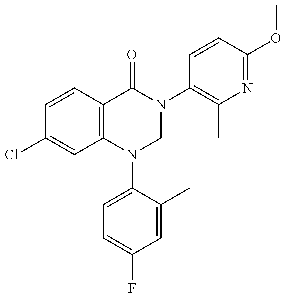 [SMILES: COc1ccc(N2CN(c3ccc(F)cc3C)c3cc(Cl)ccc3C2=O)c(C)n1] | MS (m/z) 412.0 (M + H)⁺ | 4-chloro-2-((4- fluroo-2- methylphenyl)a- mino)-N-(6- methoxy-2- methylpyridin-3- yl)benzamide |
| 630 | 1-(4-fluoro-2- methylphenyl)-3-(5- methoxy-3- methylpyrazin-2-yl)- 7-(trifluoromethyl)- 2,3- dihydroquinazolin- 4(1H)-one | 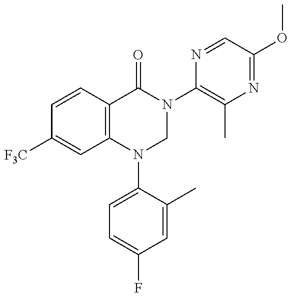 [SMILES: COc1cnc(N2CN(c3ccc(F)cc3C)c3cc(C(F)(F)F)ccc3C2=O)c(C)n1] | MS (m/z) 446.8 (M + H)⁺ | 2-((4-fluroo-2- methylphenyl)a- mino)-N-(5- methoxy-3- methylpyrazin- 2-yl)-4- (trifluoromethyl) benzamide |
| 631 | 6,7-difluoro-1-(4- fluoro-2- methylphenyl)-3-(6- methoxy-2- methylpyridin-3-yl)- 2,3- dihydroquinazolin- 4(1H)-one | 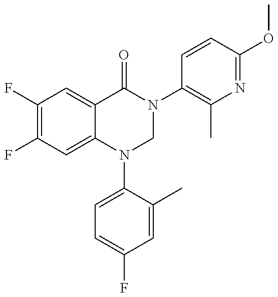 [SMILES: COc1ccc(N2CN(c3ccc(F)cc3C)c3cc(F)c(F)cc3C2=O)c(C)n1] | MS (m/z) 414.3 (M + H)⁺ | 4,5-difluoro-2- ((4-fluoro-2- methylphenyl)a- mino)-N-(6- methoxy-2- methypyridin-3- yl)benzamide |
| 632 | 3-(6-methoxy-2- methylpyridin-3-yl)- 1-(2- methylcyclohexyl)-7- (trifluoromethyl)-2,3- dihydroquinazolin- 4(1H)-one | 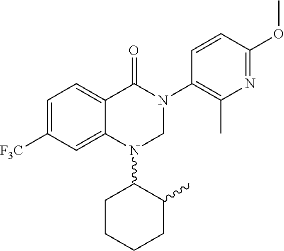 [SMILES: COc1ccc(N2CN(C3CCCCC3C)c3cc(C(F)(F)F)ccc3C2=O)c(C)n1] | MS (m/z) 434.4 (M + H)⁺ | N-(6-methoxy- 2-methylpyridin- 3-yl)-2-((2- methylcyclohex- yl)amino)-4- (trifluromethyl) benzamide |
| 633 | 6,7-difluoro-1-(4- fluoro-2- isopropylphenyl)-3- (6-methoxy-2- methylpyridin-3-yl)- 2,3- dihydroquinazolin- 4(1H)-one | 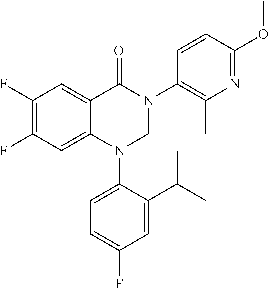 [SMILES: COc1ccc(N2CN(c3ccc(F)cc3C(C)C)c3cc(F)c(F)cc3C2=O)c(C)n1] | MS (m/z) 442.3 (M + H)⁺ | 4,5-difluoro-2- ((4-fluoro-2- isopropylphenyl) amino)-N-(6- methoxy-2- methylpyridin-3- ylbenzamide |
| 634 | 3-(6-methoxy-2- methylpyridin-3-yl)- 1-((1S,2R)-2- methylcyclohexyl)-7- (trifluoromethyl)-2,3- dihydroquinazolin- 4(1H)-one | 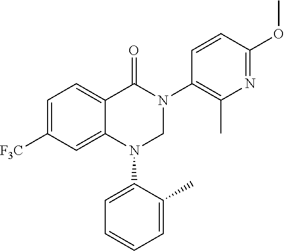 | MS (m/z) 434.3 (M + H)⁺ | N-(6-methoxy- 2-methylpyridin- 3-yl)-2- (((1S,2R)-2- methylcyclohex- yl)amino)-4- (trifluoromethyl) benzamide |
| 635 | 3-(2,6- dimethoxypyrimidin- 4-yl)-1-(4-fluoro-2- methylphenyl)-7- (trifluoromethyl)-2,3- dihydroquinazolin- 4(1H)-one | 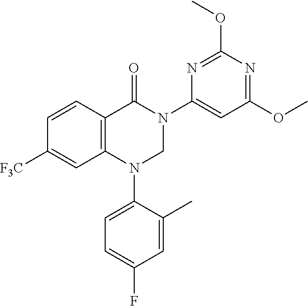 [SMILES: COc1cc(N2CN(c3ccc(F)cc3C)c3cc(C(F)(F)F)ccc3C2=O)nc(OC)n1] | MS (m/z) 463.0 (M + H)⁺ | N-(2,6- dimethoxypyrim- idin-4-yl)-2-((4- fluoro-2- methylphenyl)a- mino)-4- (trifluoromethyl) benzamide |
| 636 | 1-(2-ethyl-4- fluorophenyl)-3-(6- methoxy-2- methylpyridin-3-yl)- 6-(trifluoromethyl)- 2,3- dihydroquinazolin- 4(1H)-one | 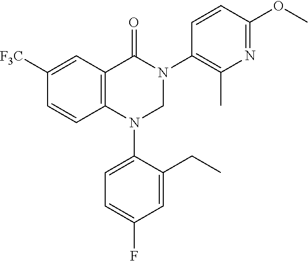 [SMILES: CCc1cc(F)ccc1N1CN(c2ccc(OC)nc2C)C(=O)c2cc(C(F)(F)F)ccc21] | MS (m/z) 460.0 (M + H)⁺ | 2-((2-ethyl-4- fluorophenyl)ami- no)-N-(6- methoxy-2- methylpyridin-3- yl)-5- (trifluoromethyl) benzamide |
| 637 | 3-(6-methoxy-2- methylpyridin-3-yl)- 1-(4-methylthiazol-5- yl)-6- (trifluoromethyl)-2,3- dihydroquinazolin- 4(1H)-one | 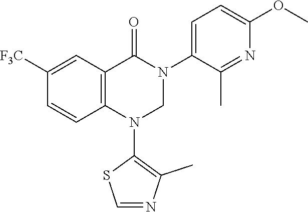 [SMILES: COc1ccc(N2CN(c3scnc3C)c3ccc(C(F)(F)F)cc3C2=O)c(C)n1] | MS (m/z) 434.8 (M)⁺ | N-(6-methoxy- 2-methylpyridin- 3-yl)-2-((4- methylthiazol-5- yl)amino)-5- (trifluoromethyl) benzamide |
| 638 | 6-chloro-1-(4-fluoro- 2-methylphenyl)-3- (2-methoxy-4- methylpyrimidin-5- yl)-2,3- dihydroquinazolin- 4(1H)-one | 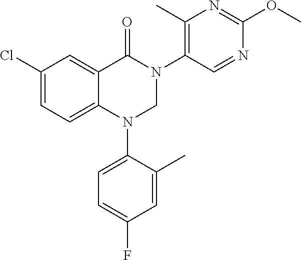 [SMILES: COc1ncc(N2CN(c3ccc(F)cc3C)c3ccc(Cl)cc3C2=O)c(C)n1] | MS (m/z) 413.0 (M + H)⁺ | 5-chloro-2-((4- fluoro-2- methylphenyl)a- mino)-N-(2- methoxy-4- methylpyrimidin- 5-yl)benzamide |
| 639 | 1-(4-fluoro-2- methylphenyl)-3-(6- methoxy-2- methylpyridin-3-yl)- 4-oxo-7- (trifluoromethyl)- 1,2,3,4- tetrahydroquinazoline- 6-carbonitrile | 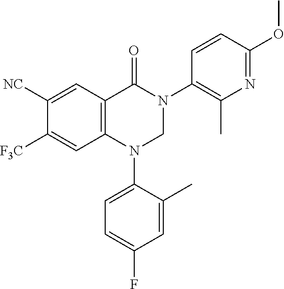 [SMILES: COc1ccc(N2CN(c3ccc(F)cc3C)c3cc(C(F)(F)F)c(C#N)cc3C2=O)c(C)n1] | MS (m/z) 471.3 (M + H)⁺ | 5-cyano-2-((4- fluoro-2- methylphenyl)a- mino)-N-(6- methoxy-2- methylpyridin-3- yl)-4- (trifluoromethyl) benzamide |
| 640 | 6-chloro-7- (difluoromethoxy)-1- (4-fluoro-2- methylphenyl)-3-(6- methoxy-2- methylpyridin-3-yl)- 2,3- dihydroquinazolin- 4(1H)-one | 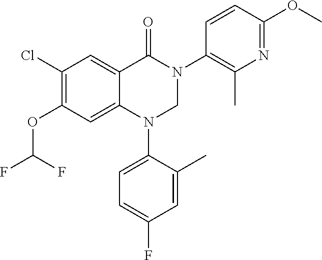 [SMILES: COc1ccc(N2CN(c3ccc(F)cc3C)c3cc(OC(F)F)c(Cl)cc3C2=O)c(C)n1] | MS (m/z) 478.3 (M + H)⁺ | 5-chloro-4- (difluoromethoxy)- 2-((4-fluoro- 2- methylphenyl)a- mino)-N-(6- methoxy-2- methylpyridin-3- yl)benzamide |
| 641 | 6-chloro-7- (difluoromethyl)-1- (4-fluoro-2- methylphenyl)-3-(6- methoxy-2- methylpyridin-3-yl)- 2,3- dihydroquinazolin- 4(1H)-one | 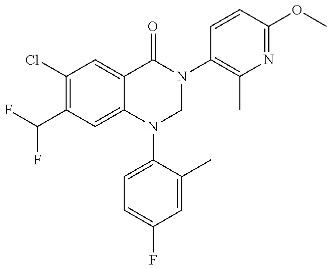 [SMILES: COc1ccc(N2CN(c3ccc(F)cc3C)c3cc(C(F)F)c(Cl)cc3C2=O)c(C)n1] | MS (m/z) 462.3 (M + H)⁺/ 464.3 (M + 3H)⁺ | 5-chloro-4- (difluoromethyl)- 2-((4-fluoro-2- methylphenyl)a- mino)-N-(6- methoxy-2- methylpyridin-3- yl)benzamide |
| 642 | 3-(6-bromo-2- methylpyridin-3-yl)- 1-(4-fluoro-2- methylphenyl)-6- (trifluoromethyl)-2,3- dihydroquinazolin- 4(1H)-one | 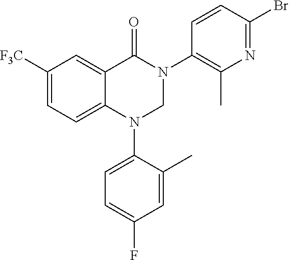 [SMILES: Cc1cc(F)ccc1N1CN(c2ccc(Br)nc2C)C(=O)c2cc(C(F)(F)F)ccc21] | MS (m/z) 494.2 (M + H)⁺/496.2 (M + 3H)⁺ | N-(6-bromo-2- methylpyridin-3- yl)-2-((4-fluoro- 2- methylphenyl)a- mino)-5- (trifluoromethyl) benzamide |
| 643 | 6-chloro-3-(6- methoxy-2- methylpyridin-3-yl)- 1-(2-methyl-4- (trifluoromethoxy)phen- yl)-4-oxo-1,2,3,4- tetrahydropyrido[2,3- d]pyrimidine-7- carbonitrile | 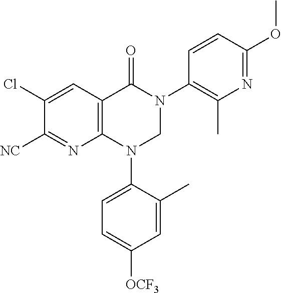 [SMILES: COc1ccc(N2CN(c3ccc(OC(F)(F)F)cc3C)c3nc(C#N)c(Cl)cc3C2=O)c(C)n1] | MS (m/z) 504.2 (M + H)⁺ | 5-chloro-6- cyano-N-(6- methoxy-2- methylpyridin-3- yl)-2-((2-methyl- 4- (trifluoromethoxy) phenyl)amino) nicotinamide |
| 644 | 1-(2,4- dimethoxyphenyl)-3- (6-methoxy-2- methylpyridin-3-yl)- 6-(trifluoromethyl)- 2,3- dihydroquinazolin- 4(1H)-one | 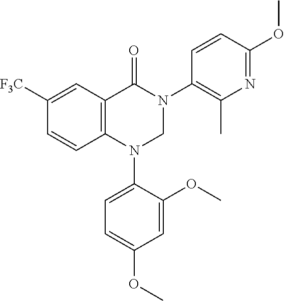 [SMILES: COc1ccc(N2CN(c3ccc(OC)nc3C)C(=O)c3cc(C(F)(F)F)ccc32)c(OC)c1] | MS (m/z) 474.3 (M + H)⁺ | 2-((2,4- dimethoxyphen- yl)amino)-N-(6- methoxy-2- methylpyridin-3- yl)-5- (trifluoromethyl) benzamide |
| 645 | 1-(4-fluoro-2- methoxyphenyl)-3- (6-methoxy-2- methylpyridin-3-yl)- 6-(trifluoromethyl)- 2,3- dihydroquinazolin- 4(1H)-one | 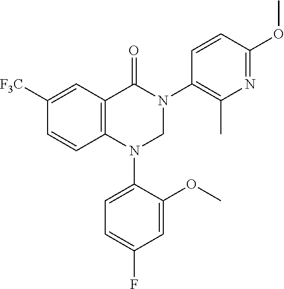 [SMILES: COc1ccc(N2CN(c3ccc(F)cc3OC)c3ccc(C(F)(F)F)cc3C2=O)c(C)n1] | MS (m/z) 462.3 (M + H)⁺ | 2-((4-fluoro-2- methoxyphenyl) amino)-N-(6- methoxy-2- methylpyridin-3- yl)-5- (trifluoromethyl) benzamide |
| 646 | 6-chloro-1-(4-fluoro- 2-methylphenyl)-3- (6-methoxy-2- methylpyridin-3-yl)- 4-oxo-1,2,3,4- tetrahydropyrido[2,3- d]pyrimidine-7- carbonitrile | 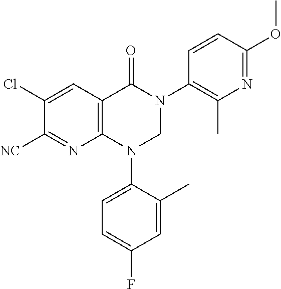 [SMILES: COc1ccc(N2CN(c3ccc(F)cc3C)c3nc(C#N)c(Cl)cc3C2=O)c(C)n1] | MS (m/z) 438.3 (M + H)⁺ | 5-chloro-6- cyano-2-((4- fluoro-2- methylphenyl)a- mino)-N-(6- methoxy-2- methylpyridin-3- yl)nicotinamide |
| 647 | 6-chloro7-fluoro-3- (6-methoxy-2- methylpyridin-3-yl)- 1-(2-methyl-4- (trifluoromethoxy)phen- yl)-2,3- dihydroquinazolin- 4(1H)-one | 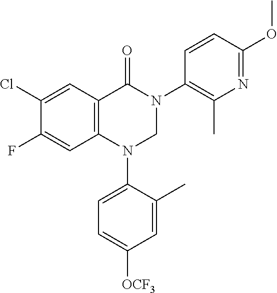 [SMILES: COc1ccc(N2CN(c3ccc(OC(F)(F)F)cc3C)c3cc(F)c(Cl)cc3C2=O)c(C)n1] | MS (m/z) 496.1 (M + H)⁺ | 5-chloro-4- fluoro-N-(6- methoxy-2- methylpyridin-3- yl)-2-((2-methyl- 4- (trifluoromethoxy) phenyl)amino) benzamide |
| 648 | 6-chloro-7-fluoro-1- (4-fluoro-2- methylphenyl)-3-(2- methoxy-4- methylpyrimidin-5- yl)-2,3- dihydroquinazolin- 4(1H)-one | 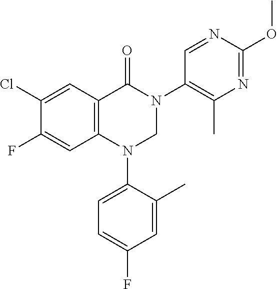 [SMILES: COc1ncc(N2CN(c3ccc(F)cc3C)c3cc(F)c(Cl)cc3C2=O)c(C)n1] | MS (m/z) 431.1 (M + H)⁺ | 5-chloro-4- fluoro-2-((4- fluoro-2- methylphenyl)a- mino)-N-(4- methoxy-4- methylpyrimidin- 5-yl)benzamide |
| 649 | 3-(6-methoxy-2- methylpyridin-3-yl)- 1-(2-methyl-3- (trifluoromethyl)phen- yl)-6- (trifluoromethyl)-2,3- dihydropyrido[2,3- d]pyrimidin-4(1H)- one | 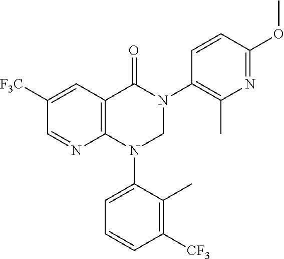 [SMILES: COc1ccc(N2CN(c3cccc(C(F)(F)F)c3C)c3ncc(C(F)(F)F)cc3C2=O)c(C)n1] | MS (m/z) 497.3 (M + H)⁺ | N-(6-methoxy- 2-methylpyridin- 3-yl)-2-((2- methyl-3- (trifluoromethyl) phenyl)amino)- 5- (trifluoromethyl) nicotinamide |
| 650 | 1-(3-chloro-2- methylphenyl)-3-(6- methoxy-2- methylpyridin-3-yl)- 6-(trifluoromethyl)- 2,3- dihydropyrido[2,3- d]pyrimidin-4(1H)- one | 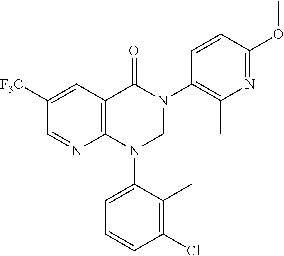 [SMILES: COc1ccc(N2CN(c3cccc(Cl)c3C)c3ncc(C(F)(F)F)cc3C2=O)c(C)n1] | MS (m/z) 463.3 (M + H)⁺ | 2-((3-chloro-2- methylphenyl)a- mino)-N-(6- methoxy-2- methylpyridin-3- yl)-5- (trifluoromethyl) nicotinamide |
| 651 | 1-(2-ethyl-4- fluorophenyl)-3-(6- methoxy-2- methylpyridin-3-yl)- 6-(trifluoromethyl)- 2,3- dihydropyrido[2,3- d]pyrimidin-4(1H)- one | 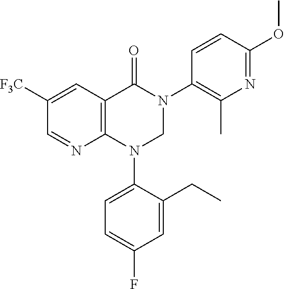 [SMILES: CCc1cc(F)ccc1N1CN(c2ccc(OC)nc2C)C(=O)c2cc(C(F)(F)F)cnc21] | MS (m/z) 461.3 (M + H)⁺ | 2-((2-ethyl-4- fluorophenyl)ami- no)-N-(6- methoxy-2- methylpyridin-3- yl)-5- (trifluoromethyl) nicotinamide |
| 652 | 1-(3,4-difluoro-2- methylphenyl)-3-(6- methoxy-2- methylpyridin-3-yl)- 6-(trifluoromethyl)- 2,3- dihydropyrido[2,3- d]pyrimidin-4(1H)- one | 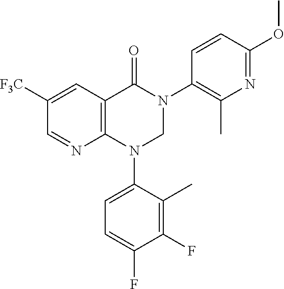 [SMILES: COc1ccc(N2CN(c3ccc(F)c(F)c3C)c3ncc(C(F)(F)F)cc3C2=O)c(C)n1] | MS (m/z) 465.3 (M + H)⁺ | 2-((3,4-difluoro- 2- methylphenyl)a- mino)-N-(6- methoxy-2- methylpyridin-3- yl)-5- (trifluoromethyl) nicotinamide |
| 653 | 1-(4-fluoro-2- isopropylphenyl)-3- (6-methoxy-2- methylpyridin-3-yl)- 6-(trifluoromethyl)- 2,3- dihydroquinazolin- 4(1H)-one | 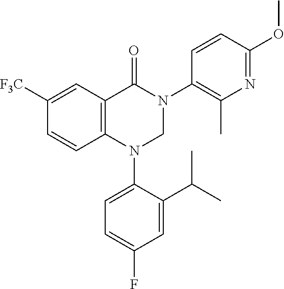 [SMILES: COc1ccc(N2CN(c3ccc(F)cc3C(C)C)c3ccc(C(F)(F)F)cc3C2=O)c(C)n1] | MS (m/z) 474.3 (M + H)⁺ | 2-((4-fluoro-2- isopropylphenyl) amino)-N-(6- methoxy-2- methylpyridin-3- yl)-5- (trifluoromethyl) benzamide |
| 654 | 7-(difluoromethoxy)- 1-(4-fluoro-2- methylphenyl)-3-(6- methoxy-2- methylpyridin-3-yl)- 2,3- dihydropyrido[2,3- d]pyrimidin-4(1H)- one | 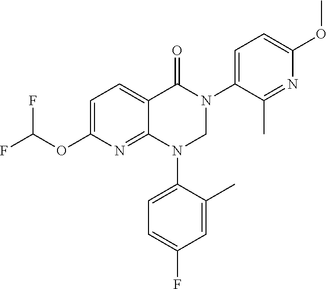 [SMILES: COc1ccc(N2CN(c3ccc(F)cc3C)c3nc(OC(F)F)ccc3C2=O)c(C)n1] | MS (m/z) 445 (M + H)⁺ | 6- (difluoromethoxy)- 2-((4-fluoro- 2- methylphenyl)a- mino)-N-(6- methoxy-2- methylpyriidin-3- yl)nicotinamide |
| 655 | 7-chloro-6-fluoro-1- (4-fluoro-2- methylphenyl)-3-(6- methoxy-2- methlypyridin-3-yl)- 2,3- dihydropyrido[2,3- d]pyrimidin-4(1H)- one | 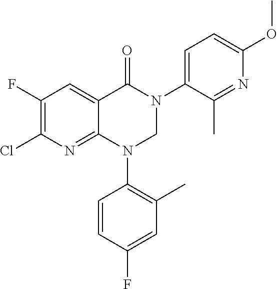 [SMILES: COc1ccc(N2CN(c3ccc(F)cc3C)c3nc(Cl)c(F)cc3C2=O)c(C)n1] | MS (m/z) 431 (M + H)⁺ | 6-chloro-5- fluoro-2-((4- fluoro-2- methylphenyl)a- mino)-N-(6- methoxy-2- methylpyridin-3- yl)nicotinamide |
| 656 | 6-fluoro-1-(4-fluoro- 2-isopropylphenyl)- 3-(6-methoxy-2- methylpyridin-3-yl)- 2,3- dihydroquinazolin- 4(1H)-one | 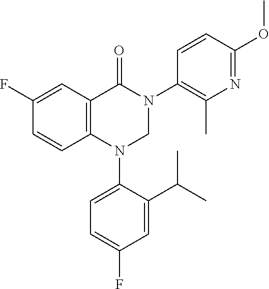 [SMILES: COc1ccc(N2CN(c3ccc(F)cc3C(C)C)c3ccc(F)cc3C2=O)c(C)n1] | MS (m/z) 424.4 (M + H)⁺ | 5-fluoro-2-((4- fluoro-2- isopropylphenyl) amino)-N-(6- methoxy-2- methylpyridin-3- yl)benzamide |
| 657 | 1-(2-cyclopropyl-4- fluorophenyl)-3-(6- methoxy-2- methylpyridin-3-yl)- 2,3- dihydroquinazolin- 4(1H)-one | 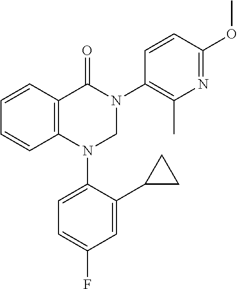 [SMILES: COc1ccc(N2CN(c3ccc(F)cc3C3CC3)c3ccccc3C2=O)c(C)n1] | MS (m/z) 404.3 (M + H)⁺ | 2-((2- cyclopropyl-4- fluorophenyl)ami- no)-N-(6- methoxy-2- methylpyridin-3- yl)benzamide |
| 658 | 1-(2-cyclopropyl-4- fluorophenyl)-3-(6- methoxy-2- methylpyridin-3-yl)- 7-(trifluoromethyl)- 2,3- dihydroquinazolin- 4(1H)-one | 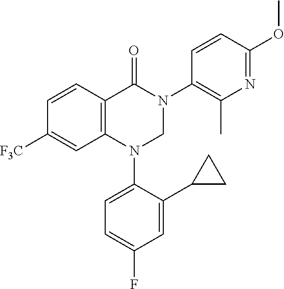 [SMILES: COc1ccc(N2CN(c3ccc(F)cc3C3CC3)c3cc(C(F)(F)F)ccc3C2=O)c(C)n1] | MS (m/z) 472.4 (M + H)⁺ | 2-((2- cyclopropyl-4- fluorophenyl)ami- no)-N-(6- methoxy-2- methylpyridin-3- yl)-4- (trifluoromethyl) benzamide |
| 659 | 5-fluoro-1-(4-fluoro- 2-methylphenyl)-3- (6-methoxy-2- methylpyridin-3-yl)- 2,3- dihydroquinazolin- 4(1H)-one | 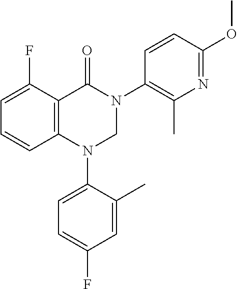 [SMILES: COc1ccc(N2CN(c3ccc(F)cc3C)c3cccc(F)c3C2=O)c(C)n1] | MS (m/z) 396.3 (M + H)⁺ | 2-fluoro-6-((4- fluuoro-2- methylphenyl)a- mino)-N-(6- methoxy-2- methylpyridin-3- yl)benzamide |
| 660 | 1-(2-ethyl-4- fluorophenyl)-3-(6- methoxy-2- methylpyridin-3-yl)- 7-(trifluoromethyl)- 2,3- dihydroquinazolin- 4(1H)-one | 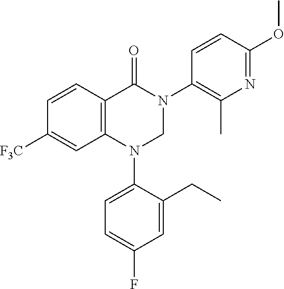 [SMILES: CCc1cc(F)ccc1N1CN(c2ccc(OC)nc2C)C(=O)c2ccc(C(F)(F)F)cc21] | MS (m/z) 460.3 (M + H)⁺ | 2-((2-ethyl-4- fluorophenyl)ami- no)-N-(6- methoxy-2- methlpyridin-3- yl)-4- (trifluoromethyl) benzamide |
| 661 | 7-(difluoromethoxy)- 1-(4-fluoro-2- methylphenyl)-3-(6- methoxy-2- methylpyridin-3-yl)- 2,3- dihydroquinazolin- 4(1H)-one | 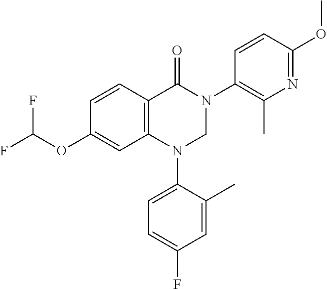 [SMILES: COc1ccc(N2CN(c3ccc(F)cc3C)c3cc(OC(F)F)ccc3C2=O)c(C)n1] | MS (m/z) 444.3 (M + H)⁺ | 4- (difluoromethoxy)- 2-((4-fluoro- 2- methylphenyl)a- mino)-N-(6- methoxy-2- methylpyridin-3- yl)benzamide |
| 662 | 7-cyclopropyl-1-(4- fluoro-2- methylphenyl)-3-(6- methoxy-2- methylpyridin-3-yl)- 2,3- dihydroquinazolin- 4(1H)-one | 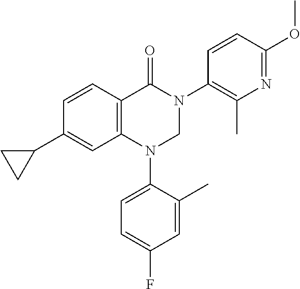 [SMILES: COc1ccc(N2CN(c3ccc(F)cc3C)c3cc(C4CC4)ccc3C2=O)c(C)n1] | MS (m/z) 418.4 (M + H)⁺ | 4-cyclopropyl-2- ((4-fluoro-2- methylphenyl)a- mino)-N-(6- methoxy-2- methylpyridin-3- yl)benzamide |
| 663 | 1-(4-fluoro-2- methylphenyl)-3-(6- methoxy-2- methylpyridin-3-yl)- 6-(trifluoromethoxy)- 2,3- dihydroquinazolin- 4(1H)-one | 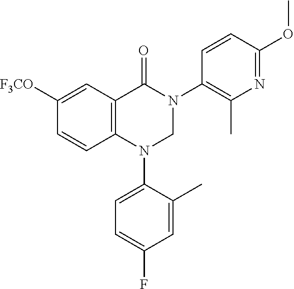 [SMILES: COc1ccc(N2CN(c3ccc(F)cc3C)c3ccc(OC(F)(F)F)cc3C2=O)c(C)n1] | MS (m/z) 462.3 (M + H)⁺ | 2-((4-fluoro-2- methylphenyl)a- mino)-N-(6- methoxy-2- methylpyridin-3- yl)-5- (trifluoromethoxy) benzamide |
| 664 | 1-(4- (difluoromethoxy)-2- methylphenyl)-3-(6- methoxy-2- methylpyridin-3-yl)- 6-(trifluoromethyl)- 2,3- dihydroquinazolin- 4(1H)-one | 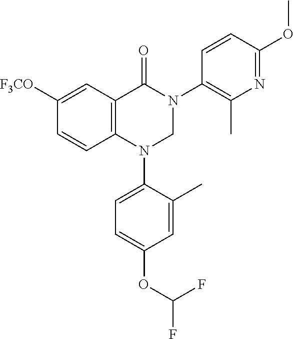 | MS (m/z) 494.3 (M + H)⁺ | 2-((4- (difluoromethoxy)- 2- methylphenyl)a- mino)-N-(6- methoxy-2- methylpyridin-3- yl)-5- (trifluoromethyl) benzamide |
| 665 | 3-(2-ethyl-6- methoxypyridin-3- yl)-1-(4-fluoro-2- methylphenyl)-7- (trifluoromethoxy)- 2,3- dihydroquinazolin- 4(1H)-one | 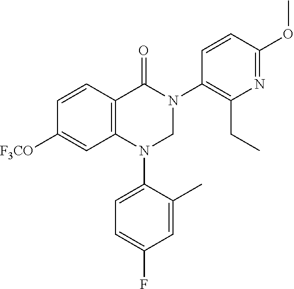 [SMILES: CCc1nc(OC)ccc1N1CN(c2ccc(F)cc2C)c2cc(OC(F)(F)F)ccc2C1=O] | MS (m/z) 476.3 (M + H)⁺ | N-(2-ethyl-6- methoxypyridin- 3-yl)-2-((4- fluoro-2- methylphenyl)a- mino)-4- (trifluoromethoxy) benzamide |
| 666 | 6-chloro-1-(2-ethyl- 4-fluorophenyl)-7- fluoro-3-(6-methoxy- 2-methylpyridin-3- yl)-2,3- dihydroquinazolin- 4(1H)-one | 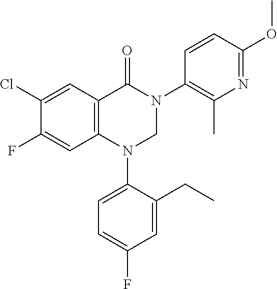 [SMILES: CCc1cc(F)ccc1N1CN(c2ccc(OC)nc2C)C(=O)c2cc(Cl)c(F)cc21] | MS (m/z) 444.3 (M + H)⁺ | 5-chloro-2-((2- ethyl-4- fluorophenyl)ami- no)-4-fluoro-N- (6-methoxy-2- methylpyridin-3- yl)benzamide |
| 667 | 1-(4-fluoro-2- methylphenyl)-3-(2- methoxy-6- ((tetrahydro-2H- pyran-2- yl)oxy)pyridin-3-yl)- 7-(trifluoromethyl)- 2,3- dihydroquinazolin- 4(1H)-one | 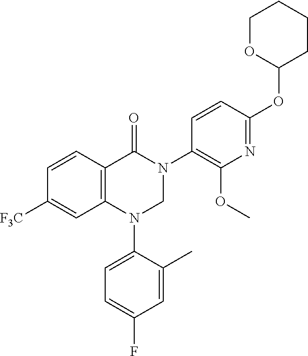 [SMILES: COc1nc(OC2CCCCO2)ccc1N1CN(c2ccc(F)cc2C)c2cc(C(F)(F)F)ccc2C1=O] | MS (m/z) 532.3 (M + H)⁺ | 2-((4-fluoro-2- methylphenyl)a- mino)-N-(2- methoxy-6- ((tetrahydro-2H- pyran-2- yl)oxy)pyridin-3- yl)-4- (trifluoromethyl) benzamide |
| 668 | 6-chloro-5,7- difluoro-1-(4-fluoro- 2-methylphenyl)-3- (6-methoxy-2- methylpyridin-3-yl)- 2,3- dihydroquinazolin- 4(1H)-one | 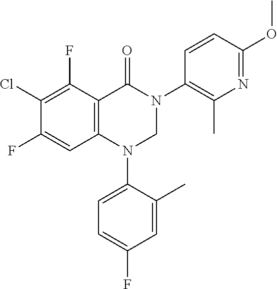 [SMILES: COc1ccc(N2CN(c3ccc(F)cc3C)c3cc(F)c(Cl)c(F)c3C2=O)c(C)n1] | MS (m/z) 448.2 (M + H)⁺ | 3-chloro-2,4- difluoro-6-((4- fluoro-2- methylphenyl)a- mino)-N-(6- methoxy-2- methylpyridin-3- yl)benzamide |
| 669 | 6-chloro-5-fluoro-1- (4-fluoro-2- methyphenyl)-3-(6- methoxy-2- methylpyridin-3-yl)- 4-oxo-1,2,3,4- tetrahydroquinazoline- 7-carbonitrile | 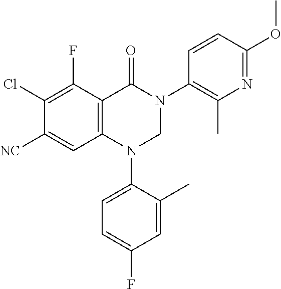 [SMILES: COc1ccc(N2CN(c3ccc(F)cc3C)c3cc(C#N)c(Cl)c(F)c3C2=O)c(C)n1] | MS (m/z) 455.3 (M + H)⁺ | 3-chloro-4- cyano-2-fluoro- 6-((4-fluoro-2- methylphenyl)a- mino)-N-(6- methoxy-2- methylpyridin-3- yl)benzamide |
| 670 | 3-(6-methoxy-2- methylpyridin-3-yl)- 1-(2-methyl-4- (trifluoromethyl)phen- yl)-6- (trifluoromethyl)-2,3- dihydropyrido[2,3- d]pyrimidin-4(1H)- one | 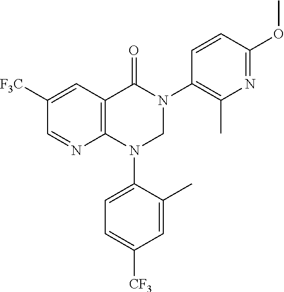 [SMILES: COc1ccc(N2CN(c3ccc(C(F)(F)F)cc3C)c3ncc(C(F)(F)F)cc3C2=O)c(C)n1] | MS (m/z) 497.3 (M + H)⁺ | N-(6-methoxy- 2-methylpyridin- 3-yl)-2-((2- methyl-4- (trifluoromethyl) phenyl)amino)- 5- (trifluoromethyl) nicotinamide |
| 671 | 1-(4-chloro-2- methylphenyl)-3-(6- methoxy-2- methylpyridin-3-yl)- 6-(trifluoromethyl)- 2,3- dihydropyrido[2,3- d]pyrimidin-4(1H)- one | 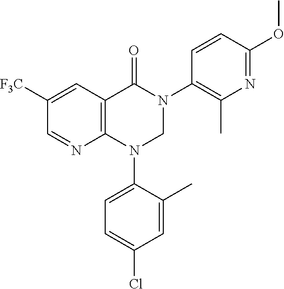 [SMILES: COc1ccc(N2CN(c3ccc(Cl)cc3C)c3ncc(C(F)(F)F)cc3C2=O)c(C)n1] | MS (m/z) 463.3 (M + H)⁺ | 2-((4-chloro-2- methylphenyl)a- mino)-N-(6- methoxy-2- methylpyridin-3- yl)-5- (trifluoromethyl) nicotinamide |
| 672 | 1-(2-bromo-4- fluorophenyl)-6- chloro-3-(6- methoxy-2- methylpyridin-3-yl)- 2,3- dihydroquinazolin- 4(1H)-one | 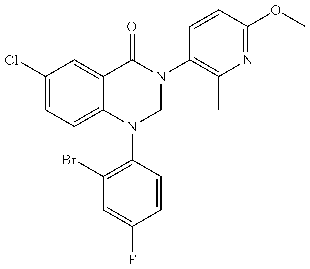 [SMILES: COc1ccc(N2CN(c3ccc(F)cc3Br)c3ccc(Cl)cc3C2=O)c(C)n1] | MS (m/z) 478.2 (M + 3H)⁺ | 2-((2-bromo-4- fluorophenyl)ami- no)-5-chloro-N- (6-methoxy-2- methlypyridin-3- yl)benzamide |
| 673 | 1-(4-fluoro-2- methylphenyl)-3-(2- methoxypyridin-3- yl)-7- (trifluoromethyl)-2,3- dihydroquinazolin- 4(1H)-one | 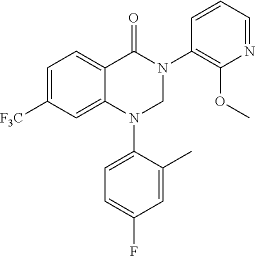 [SMILES: COc1ncccc1N1CN(c2ccc(F)cc2C)c2cc(C(F)(F)F)ccc2C1=O] | MS (m/z) 432.3 (M + H)⁺ | 2-((4-fluoro-2- methylphenyl)a- mino)-N-(2- methoxypyridin- 3-yl)-4- (trifluoromethyl) benzamide |
| 674 | 1-(4-fluoro-2- methylphenyl)-3-(2- methoxy-4- methylpyrimidin-5- yl)-7- (trifluoromethoxy)- 2,3- dihydroquinazolin- 4(1H)-one | 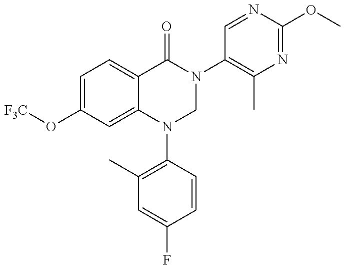 [SMILES: COc1ncc(N2CN(c3ccc(F)cc3C)c3cc(OC(F)(F)F)ccc3C2=O)c(C)n1] | MS (m/z) 463.3 (M + H)⁺ | 2-((4-fluoro-2- methylphenyl)a- mino)-N-(2- methoxy-4- methylpyrimidin- 5-yl)-4- (trifluoromethoxy) benzamide |
| 675 | 1-(4-fluoro-2- methylphenyl)-3-(6- methoxy-2- methylpyridin-3-yl)- 7-methyl-2,3- dihydropyrido[2,3- d]pyrimidin-4(1H)- one | 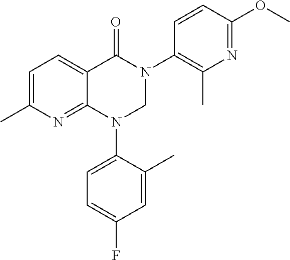 | MS (m/z) 393.4 (M + H)⁺ | 2-((4-fluoro-2- methylphenyl)a- mino)-N-(6- methoxy-2- methylpyridin-3- y)-6- methynicotina- mide |
| 676 | 6-chloro-1-(2-ethyl- 4-fluorophenyl)-3-(6- methoxy-2- methylpyridin-3-yl)- 4-oxo-1,2,3,4- tetrahydropyrido[2,3- d]pyrimidine-7- carbonitrile | 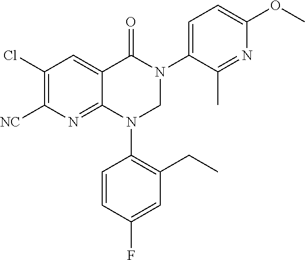 [SMILES: CCc1cc(F)ccc1N1CN(c2ccc(OC)nc2C)C(=O)c2cc(Cl)c(C#N)nc21] | MS (m/z) 452.3 (M + H)⁺ | 5-chloro-6- cyano-2-((2- ethyl-4- fluorophenyl)ami- no)-N-(6- methoxy-2- methylpyridin-3- yl)nicotinamide |
| 677 | 3-(2-bromo-6- methoxy-4- methylpyridin-3-yl)- 1-(4-fluoro-2- methylphenyl)-7- (trifluoromethyl)-2,3- dihydroquinazolin- 4(1H)-one | 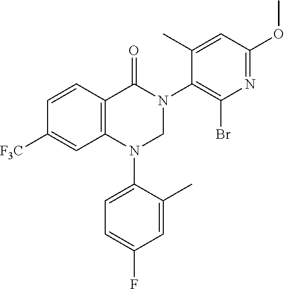 [SMILES: COc1cc(C)c(N2CN(c3ccc(F)cc3C)c3cc(C(F)(F)F)ccc3C2=O)c(Br)n1] | MS (m/z) 524.2 (M + H)⁺ | N-(2-bromo-6- methoxy-4- methylpyridin-3- yl)-2-((4-fluoro- 2- methylphenyl)a- mino)-4- (trifluoromethyl) benzamide |
| 678 | 1-(4-fluoro-2- isopropylphenyl)-3- (6-methoxy-2- methylpyridin-3-yl)- 7-(trifluoromethyl)- 2,3- dihydropyrido[2,3- d]pyrimidin-4(1H)- one | 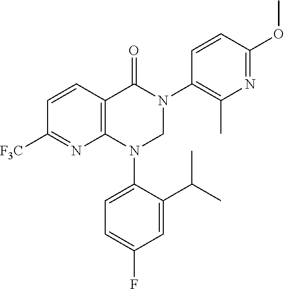 [SMILES: COc1ccc(N2CN(c3ccc(F)cc3C(C)C)c3nc(C(F)(F)F)ccc3C2=O)c(C)n1] | MS (m/z) 475.3 (M + H)⁺ | 2-((4-fluoro-2- isopropylphenyl) amino)-N-(6- methoxy-2- methylpyridin-3- yl)-6- (trifluoromethyl) nicotinamide |
| 679 | 6-chloro-1-(4-fluoro- 2-isopropylphenyl)- 3-(6-methoxy-2- methylpyridin-3-yl)- 2,3- dihydropyrido[2,3- d]pyrimidin-4(1H)- one | 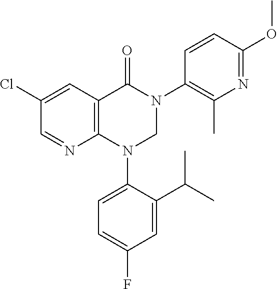 [SMILES: COc1ccc(N2CN(c3ccc(F)cc3C(C)C)c3ncc(Cl)cc3C2=O)c(C)n1] | MS (m/z) 441.3 (M + H)⁺ | 5-chloro-2-((4- fluoro-2- isopropylphenyl) amino)-N-(6- methoxy-2- methylpyridin-3- yl)nicotinamide |
| 680 | 1-(4-fluoro-2- isopropylphenyl)-3- (6-methoxy-2,4- dimethylpyridin-3- yl)-7- (trifluoromethyl)-2,3- dihydroquinazolin- 4(1H)-one | 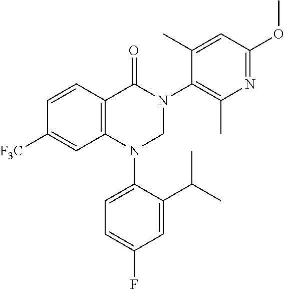 [SMILES: COc1cc(C)c(N2CN(c3ccc(F)cc3C(C)C)c3cc(C(F)(F)F)ccc3C2=O)c(C)n1] | MS (m/z) 488.1 (M + H)⁺ | 2-((4-fluoro-2- isopropylphenyl) amino)-N-(6- methoxy-2,4- dimethylpyridin- 3-yl)-4- (trifluoromethyl) benzamide |
| 681 | 1-(4-fluoro-2- isopropylphenyl)-3- (6-methoxy-2- methylpyridin-3-yl)- 6-(trifluoromethyl)- 2,3- dihydropyrido[2,3- d]pyrimidin-4(1H)- one | 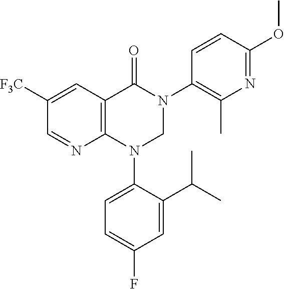 [SMILES: COc1ccc(N2CN(c3ccc(F)cc3C(C)C)c3ncc(C(F)(F)F)cc3C2=O)c(C)n1] | MS (m/z) 475.2 (M + H)⁺ | 2-((4-fluoro-2- isopropylphenyl) amino)-N-(6- methoxy-2- mehtylpyridin-3- yl)-5 (trifluoromethyl) nicotinamide |
| 682 | 3-(2-ethyl-6- methoxypyridin-3- yl)-1-(4-fluoro-2- methylphenyl)-6- (trifluoromethyl)-2,3- dihydroquinazolin- 4(1H)-one | 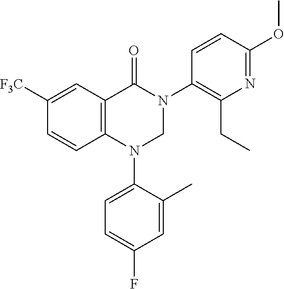 [SMILES: CCc1nc(OC)ccc1N1CN(c2ccc(F)cc2C)c2ccc(C(F)(F)F)cc2C1=O] | MS (m/z) 460.2 (M + H)⁺ | N-(2-ethyl-6- methoxypyridin- 3-yl)-2-((4- fluoro-2- methylphenyl)a- mino)-5- (trifluoromethyl) benzamide |
| 683 | 6-chloro-1-(2- ethoxy-4- fluorophenyl)-3-(6- methoxy-2- methylpyridin-3-yl)- 2,3- dihydroquinazolin- 4(1H)-one | 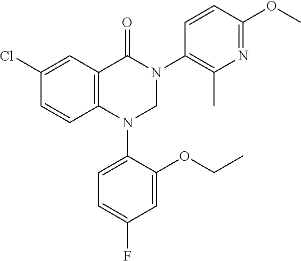 [SMILES: CCOc1cc(F)ccc1N1CN(c2ccc(OC)nc2C)C(=O)c2cc(Cl)ccc21] | MS (m/z) 441.8 (M)⁺ | 5-chloro-2-((2- ethoxy-4- fluorophenyl)amio no)-N-(6- methoxy-2- methylpyridin-3- yl)benzamide |
| 684 | 1-(4-fluoro-2- isopropylphenyl)-3- (6-methoxy-2- methylpyridin-3-yl)- 7-methyl-2,3- dihydroquinazolin- 4(1H)-one | 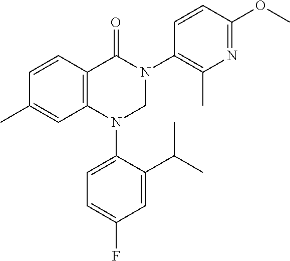 | MS (m/z) 420.2 (M + H)⁺ | 2-((4-fluoro-2- isopropylphenyl) amino)-N-(6- methoxy-2- methylpyridin-3- yl)-4- methylbenzamide |
| 685 | 7-(difluoromethyl)-6- fluoro-1-(4-fluoro-2- methylphenyl)-3-(6- methoxy-2- methylpyridin-3-yl)- 2,3- dihydroquinazolin- 4(1H)-one | 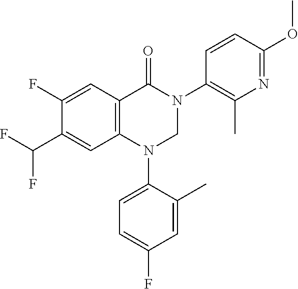 [SMILES: COc1ccc(N2CN(c3ccc(F)cc3C)c3cc(C(F)F)c(F)cc3C2=O)c(C)n1] | MS (m/z) 446.0 (M + H)⁺ | 4- (difluoromethyl)- 5-fluoro-2-((4- fluoro-2- methylphenyl)a- mino)-N-(6- methoxy-2- methylpyridin-3- yl)benzamide |
| 686 | 6-fluoro-1-(4-fluoro- 2-methylphenyl)-3- (6-methoxy-2- methylpyridin-3-yl)- 7-methyl-2,3- dihydropyrido[2,3- d]pyrimidin-4(1H)- one | 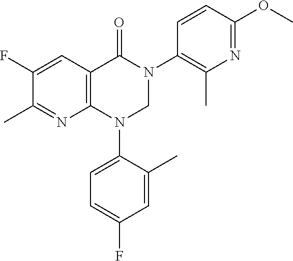 | MS (m/z) 411.0 (M + H)⁺ | 5-fluoro-2-((4- fluoro-2- methylphenyl)a- mino)-N-(6- methoxy-2- methylpyridin-3- yl)-6- methylnicotina- mide |
| 687 | 6-chloro-1-(4-fluoro- 2-methylphenyl)-3- (6-methoxy-2- methylpyridin-3-yl)- 7-methyl-2,3- dihydropyrido[2,3- d]pyrimdiin-4(1H)- one | 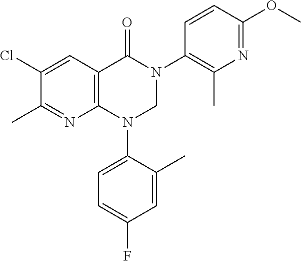 | MS (m/z) 427.0 (M + H)⁺ | 5-chloro-2-((4- fluoro-2- methylphenyl)a- mino)-N-(6- methoxy-2- methylpyridin-3- yl)-6- methylnicotina- mide |
| 688 | 6,7-dichloro-1-(4- fluoro-2- methylphenyl)-3-(6- methoxy-2- methylpyridin-3-yl)- 2,3- dihydropyrido[2,3- d]pyrimidin-4(1H)- one | 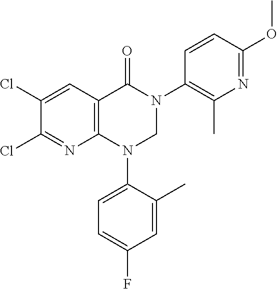 [SMILES: COc1ccc(N2CN(c3ccc(F)cc3C)c3nc(Cl)c(Cl)cc3C2=O)c(C)n1] | MS (m/z) 447.0 (M + H)⁺ | 5,6-dichloro-2- ((4-fluoro-2- methylphenyl)a- mino)-N-(6- methoxy-2- methylpyridin-3- yl)nicotinamide |
| 689 | 6-chloro-5-fluoro-3- (6-methoxy-2- methylpyridin-3-yl)- 1-(2-methyl-4- (trifluoromethoxy)phen- yl)-2,3- dihydroquinazolin- 4(1H)-one | 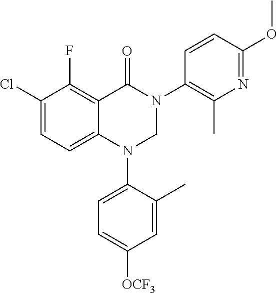 [SMILES: COc1ccc(N2CN(c3ccc(OC(F)(F)F)cc3C)c3ccc(Cl)c(F)c3C2=O)c(C)n1] | MS (m/z) 496.0 (M + H)⁺ | 3-chloro-2- fluoro-N-(6- methoxy-2- methylpyridin-3- yl)-6-((2-methyl- 4- (trifluoromethoxy) phenyl)amino) benzamide |
| 690 | 7-(dimethylamino)-1- (4-fluoro-2- methylphenyl)-3-(6- methoxy-2- methylpyridin-3-yl)- 2,3- dihydroquinazolin- 4(1H)-one | 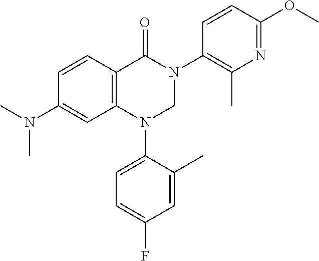 | MS (m/z) 421.2 (M + H)⁺ | 4- (dimethylamino)- 2-((4-fluoro-2- methylphenyl)a- mino)-N-(6- methoxy-2- methylpyridin-3- yl)benzamide |
| 691 | 1-(4-fluoro-2- methylphenyl)-3-(6- methoxy-2- methylpyridin-3-yl)- 4-oxo-1,2,3,4- tetrahydroquinazoline- 6-carbonitrile | 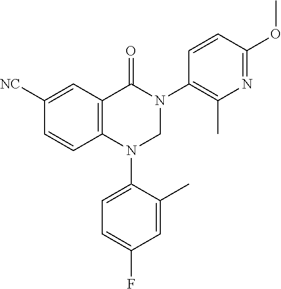 [SMILES: COc1ccc(N2CN(c3ccc(F)cc3C)c3ccc(C#N)cc3C2=O)c(C)n1] | MS (m/z) 403.2 (M + H)⁺ | 5-cyano-2-((4- fluoro-2- methylphenyl)a- mino)-N-(6- methoxy-2- methylpyridin-3- yl)benzamide |
| 692 | 6-chloro-1-(4-fluoro- 2-isopropylphenyl)- 3-(6-methoxy-2- methylpyridin-3-yl)- 2,3- dihydroquinazolin- 4(1H)-one | 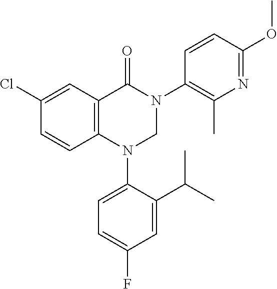 [SMILES: COc1ccc(N2CN(c3ccc(F)cc3C(C)C)c3ccc(Cl)cc3C2=O)c(C)n1] | MS (m/z) 440.1 (M + H)⁺ | 5-chloro-2-((4- fluoro-2- isopropylphenyl) amino)-N-(6- methoxy-2- methypyridin-3- yl)benzamide |
| 693 | 1-(4-fluoro-3- methoxy-2- methylphenyl)-3-(6- methoxy-2- methylpyridin-3-yl)- 7-(trifluoromethyl)- 2,3- dihydroquinazolin- 4(1H)-one | 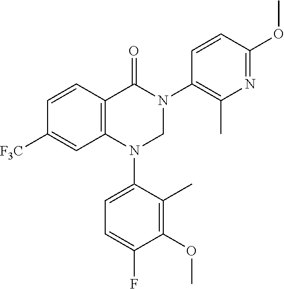 [SMILES: COc1ccc(N2CN(c3ccc(F)c(OC)c3C)c3cc(C(F)(F)F)ccc3C2=O)c(C)n1] | MS (m/z) 475.8 (M + H)⁺ | 2-((4-fluoro-3- methoxy-2- methylphenyl)a- mino)-N-(6- methoxy-2- methylpyridin-3- yl)-4- (trifluoromethyl) benzamide |
| 694 | 6-chloro-5-fluoro-1- (4-fluoro-2- methylphenyl)-3-(6- methoxypyridin-3- yl)-2,3- dihydroquinazolin- 4(1H)-one | 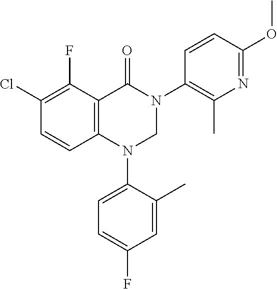 | MS (m/z) 416.0 (M + H)⁺ | 3-chloro-2- fluoro-6-((4- fluoro-2- methylphenyl)a- mino)-N-(6- methoxypyridin- 3-yl(benzamide |
| 695 | 6-chloro-5-fluoro-1- (4-fluoro-2- methylphenyl)-3-(6- methoxy-2- methylpyridin-3-yl)- 2,3- dihydroquinazolin- 4(1H)-one | 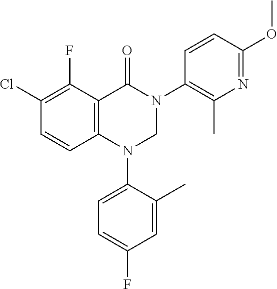 [SMILES: COc1ccc(N2CN(c3ccc(F)cc3C)c3ccc(Cl)c(F)c3C2=O)c(C)n1] | MS (m/z) 430.0 (M + H)⁺ | 3-chloro-2- fluoro-6-((4- fluoro-2- methylphenyl)a- mino)-N-(6- methoxy-2- methylpyridin-3- yl)benzamide |
| 696 | 3-(6-methoxy-2- methylpyridin-3-yl)- 1-(3-methylthiophen- 2 yl)-6- (trifluoromethyl)-2,3- dihydroquinazolin- 4(1H)-one | 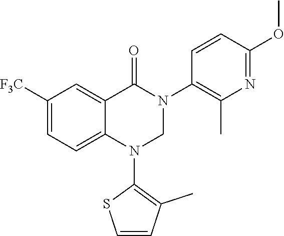 [SMILES: COc1ccc(N2CN(c3sccc3C)c3ccc(C(F)(F)F)cc3C2=O)c(C)n1] | MS (m/z) 433.8 (M)⁺ | N-(6-methoxy- 2-methylpyridin- 3-yl)-2-((3- methylthiophen- 2-yl)amino)-5- (trifluoromethyl) benzamide |
| 697 | 1-(4-fluoro-2- methoxyphenyl)-3- (6-methoxypyridin-3- yl)-4-oxo-1,2,3,4- tetrahydroquinazoline- 7-carbonitrile | 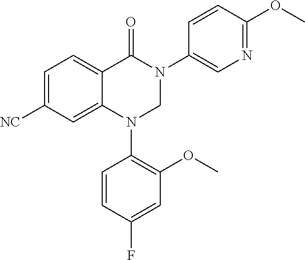 [SMILES: COc1ccc(N2CN(c3ccc(F)cc3OC)c3cc(C#N)ccc3C2=O)cn1] | MS (m/z) 405.0 (M + H)⁺ | 4-cyano-2-((4- fluoro-2- methoxyphenyl) amino)-N-(6- methoxypyridin- 3-yl)benzamide |
| 698 | 1-(4-fluoro-2- methylphenyl)-3-(6- methoxy-2- methylpyridin-3-yl)- 7-(methylsulfonyl)- 2,3- dihydroquinazolin- 4(1H)-one | 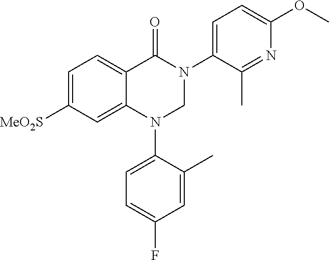 [SMILES: COc1ccc(N2CN(c3ccc(F)cc3C)c3cc(S(C)(=O)=O)ccc3C2=O)c(C)n1] | MS (m/z) 455.8 (M)⁺ | 2-((4-fluoro-2- methylphenyl)a- mino)-N-(6- methoxy-2- methylpyridin-3- yl)-4- (methylsulfonyl) benzamide |
| 699 | 6-chloro-1-(2-ethyl- 4-fluorophenyl)-3-(6- methoxy-2- methylpyridin-3-yl)- 2,3- dihydroquinazolin- 4(1H)-one | 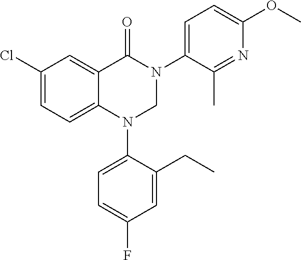 [SMILES: CCc1cc(F)ccc1N1CN(c2ccc(OC)nc2C)C(=O)c2cc(Cl)ccc21] | MS (m/z) 426.0 (M + H)⁺ | 5-chloro-2-((2- ethyl-4- fluorophenyl)ami- no)-N-(6- methoxy-2- methylpyridin-3- yl)benzamide |
| 700 | 6-chloro-1-(4-fluoro- 2-isopropylphenyl)- 3-(6-methoxy-2,4- dimethylpyridin-3- yl)-2,3- dihydroquinazolin- 4(1H)-one | 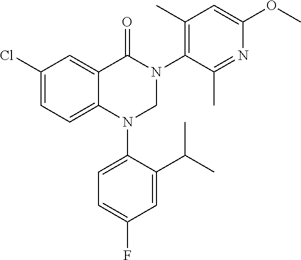 [SMILES: COc1cc(C)c(N2CN(c3ccc(F)cc3C(C)C)c3ccc(Cl)cc3C2=O)c(C)n1] | MS (m/z) 454.2 (M + H)⁺ | 5-chloro-2-((4- fluoro-2- isopropylphenyl) amino)-N-(6- methoxy-2,4- dimethylpyridin- 3-yl)benzamide |
| 701 | 1-(4-fluoro-2- methylphenyl)-3-(6- methoxy-2- methylpyridin-3-yl)- 7-(2,2,2- trifluoroethoxy)-2,3- dihydroquinazolin- 4(1H)-one | 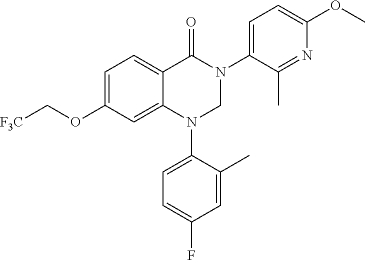 [SMILES: COc1ccc(N2CN(c3ccc(F)cc3C)c3cc(OCC(F)(F)F)ccc3C2=O)c(C)n1] | MS (m/z) 476.0 (M + H)⁺ | 2-((4-fluoro-2- methylphenyl)a- mino)-N-(6- methoxy-2- methylpyridin-3- yl)-4-(2,2,2- trifluoroethoxy)benz- amide |
| 702 | 3-(2-ethyl-6- methoxypyridin-3- yl)-1-(4-fluoro-2- isopropylphenyl)-7- (trifluoromethyl)-2,3- dihydroquinazolin- 4(1H)-one | 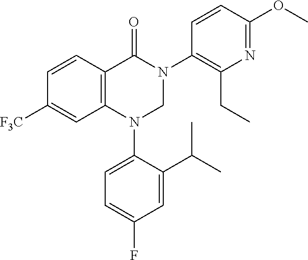 [SMILES: CCc1nc(OC)ccc1N1CN(c2ccc(F)cc2C(C)C)c2cc(C(F)(F)F)ccc2C1=O] | MS (m/z) 488.2 (M + H)⁺ | N-(2-ethyl-6- methoxypyridin- 3-yl)-2-((4- fluoro-2- isopropylphenyl) amino)-4- (trifluoromethyl) benzamide |
| 703 | 1-(4-fluoro-2- methylphenyl)-3-(5- methoxypyrazin-2- yl)-7- (trifluoromethyl)-2,3- dihydroquinazolin- 4(1H)-one | 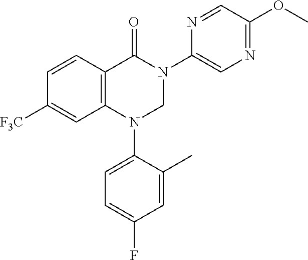 [SMILES: COc1cnc(N2CN(c3ccc(F)cc3C)c3cc(C(F)(F)F)ccc3C2=O)cn1] | MS (m/z) 432.7 (M + H)⁺ | 2-((4-fluoro-2- methylphenyl)a- mino)-N-(5- methoxypyrazin- 2-yl)-4- (trifluoromethyl) benzamide |
| 704 | 6-fluoro-1-(4-fluoro- 2-methylphenyl)-3- (6-methoxy-2- methylpyridin-3-yl)- 7-methyl-2,3- dihydroquinazolin- 4(1H)-one | 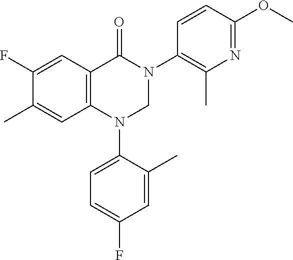 | MS (m/z) 410.0 (M + H)⁺ | 5-fluoro-2-((4- fluoro-2- methylphenyl)a- mino)-N-(6- methoxy-2- methylpyridin-3- yl)-4- methylbenzamide |
| 705 | 5-chloro-1-(4-fluoro- 2-methylphenyl)-3- (6-methoxy-2- methylpyridin-3-yl)- 2,3- dihydroquinazolin- 4(1H)-one | 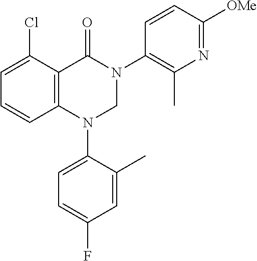 [SMILES: COc1ccc(N2CN(c3ccc(F)cc3C)c3cccc(Cl)c3C2=O)c(C)n1] | MS (m/z) 412.0 (M + H)⁺ | 2-chloro-6-((4- fluoro-2- methylphenyl)a- mino)-N-(6- methoxy-2- methylpyridin-3- yl)benzamide |
| 706 | 6-chloro-1-(4-fluoro- 2-methoxyphenyl)-3- (6-methoxy-2- methylpyridin-3-yl)- 2,3- dihydroquinazolin- 4(1H)-one | 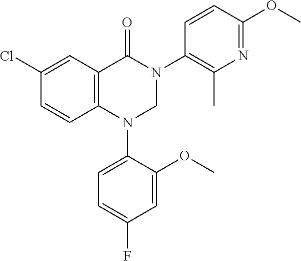 [SMILES: COc1ccc(N2CN(c3ccc(F)cc3OC)c3ccc(Cl)cc3C2=O)c(C)n1] | MS (m/z) 428.0 (M + H)⁺ | 5-chloro-2-((4- fluoro-2- methoxyphenyl) amino)-N-(6- methoxy-2- methylpyridin-3- yl)benzamide |
| 707 | 1-(4-fluoro-2- methylphenyl)-3-(3- methoxy-1-methyl- 1H-pyrazol-5-yl)-7- (trifluoromethyl)-2,3- dihydroquinazolin- 4(1H)-one | 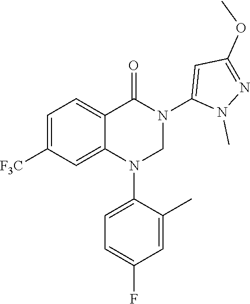 [SMILES: COc1cc(N2CN(c3ccc(F)cc3C)c3cc(C(F)(F)F)ccc3C2=O)n(C)n1] | MS (m/z) 435.0 (M + H)⁺ | 2-((4-fluoro-2- methylphenyl)a- mino)-N-(3- methoxy-1- methyl-1H- pyrazol-5-yl)-4 (trifluoromethyl) benzamide |
| 708 | 7-fluoro-1-(4-fluoro- 2-isopropylphenyl)- 3-(6-methoxy-2- methylpyridin-3-yl)- 2,3- dihydroquinazolin- 4(1H)-one | 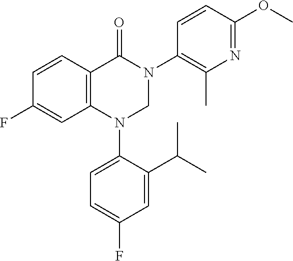 [SMILES: COc1ccc(N2CN(c3ccc(F)cc3C(C)C)c3cc(F)ccc3C2=O)c(C)n1] | MS (m/z) 424.0 (M + H)⁺ | 4-fluoro-2-((4- fluoro-2- isopropylphenyl) amino)-N-(6- methoxy-2- methylpyrdin-3- yl)benzamide |
| 709 | 1-(2-(tert-butyl)-4- fluorophenyl)-6- chloro-3-(6- methoxy-2- methylpyridin-3-yl)- 2,3- dihydroquinazolin- 4(1H)-one | 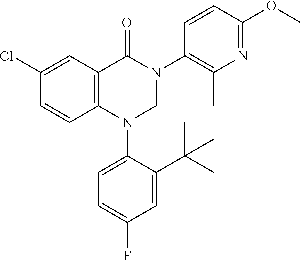 [SMILES: COc1ccc(N2CN(c3ccc(F)cc3C(C)(C)C)c3ccc(Cl)cc3C2=O)c(C)n1] | MS (m/z) 454.0 (M + H)⁺ | 2-((2-(tert- butyl)-4- fluorophenyl)ami- no)-5-chloro-N- (6-methoxy-2- methylpyridin-3- yl)benzamide |
| 710 | 1-(4-fluoro-2- methylphenyl)-3-(2- methoxy-4,6- dimethylpyrimidin-5- yl)-7- (trifluoromethyl)-2,3- dihydroquinazolin- 4(1H)-one | 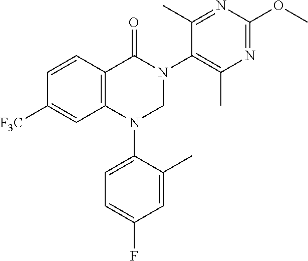 [SMILES: COc1nc(C)c(N2CN(c3ccc(F)cc3C)c3cc(C(F)(F)F)ccc3C2=O)c(C)n1] | MS (m/z) 461.0 (M + H)⁺ | 2-((4-fluoro-2- methylphenyl)a- mino)-N-(2- methoxy-4,6- dimethylpyrimidin- 5-yl)-4- (trifluoromethyl) benzamide |
| 711 | 1-(3,4-difluoro-2- methylphenyl)-3-(6- methoxy-2- methylpyridin-3-yl)- 6-(trifluoromethyl)- 2,3- dihydroquinazolin- 4(1H)-one | 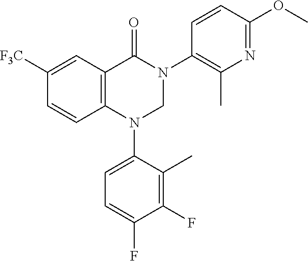 [SMILES: COc1ccc(N2CN(c3ccc(F)c(F)c3C)c3ccc(C(F)(F)F)cc3C2=O)c(C)n1] | MS (m/z) 464.0 (M + H)⁺ | 2-((3,4-difluoro- 2- methylphenyl)a- mino)-N-(6- methoxy-2- methylpyridin-3- yl)-5- (trifluoromethyl) benzamide |
| 712 | 6-chloro-3-(4-chloro- 6-methoxy-2- methylpyridin-3-yl)- 1-(4-fluoro-2- methylphenyl)-2,3- dihydroquinazolin- 4(1H)-one | 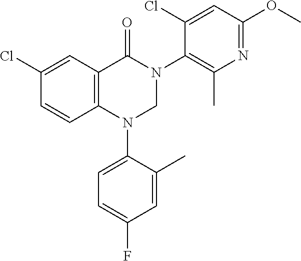 [SMILES: COc1cc(Cl)c(N2CN(c3ccc(F)cc3C)c3ccc(Cl)cc3C2=O)c(C)n1] | MS (m/z) 446.0 (M + H)⁺ | 5-chloro-N-(4- chloro-6- methoxy-2- methylpyridin-3- yl)-2-((4-fluoro- 2- methylphenyl)a- mino)benzamide |
| 713 | 6-chloro-1-(4-fluoro- 2-isopropylphenyl)- 3-(6-methoxy-2- methylpyridin-3-yl)- 5-methyl-2,3- dihydroquinazolin- 4(1H)-one | 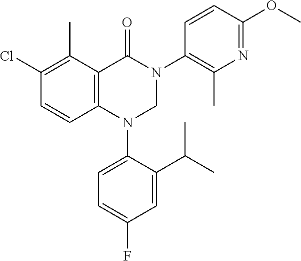 [SMILES: COc1ccc(N2CN(c3ccc(F)cc3C(C)C)c3ccc(Cl)c(C)c3C2=O)c(C)n1] | MS (m/z) 454.2 (M + H)⁺ | 3-chloro-6-((4- fluoro-2- isopropylphenyl) amino)-N-(6- methoxy-2- methypyridin-3- yl)-2- methylbenzamide |
| 714 | 6-chloro-7-fluoro-1- (4-fluoro-2- isopropylphenyl)-3- (6-methoxy-2- methylpyridin-3-yl)- 2,3- dihydroquinazolin- 4(1H)-one | 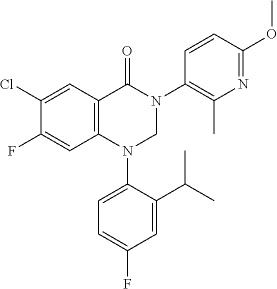 [SMILES: COc1ccc(N2CN(c3ccc(F)cc3C(C)C)c3cc(F)c(Cl)cc3C2=O)c(C)n1] | MS (m/z) 458.0 (M + H)⁺ | 5-chloro-4- fluoro-2-((4- fluoro-2- isopropylphenyl) amino)-N-(6- methoxy-2- methylpyridin-3- yl)benzamide |
| 715 | 1-(4-fluoro-2- methylphenyl)-7- methoxy-3-(2- methyl-6-oxo-1,6- dihydropyridin-3-yl)- 2,3- dihydroquinazolin- 4(1H)-one | 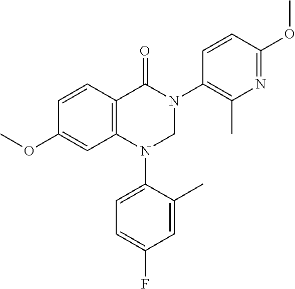 | MS (m/z) 394.2 (M + H)⁺ | 1-(4-fluoro-2- methylphenyl)- 7-methoxy-3-(6- methoxy-2- methylpyridin-3- yl)-2,3- dihydroquinazolin- 4(1H)-one |
| 716 | 1-(4-fluoro-2- methylphenyl)-6- methoxy-3-(6- methoxy-2- methylpyridin-3-yl)- 7-(trifluoromethyl)- 2,3- dihydroquinazolin- 4(1H)-one | 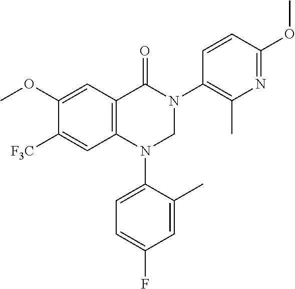 | MS (m/z) 476.1 (M + H)⁺ | 2-((4-fluoro-2- methylphenyl)a- mino)-5- methoxy-N-(6- methoxy-2- methylpyridin-3- yl)-4- (tifluoromethyl) benzamide |
| 717 | 6-chloro-1-(4-fluoro- 2-methylphenyl)-3 (6-methoxy-2- methylpyridin-3-yl)- 2,3- dihydropyrido[2,3- d]pyrimidin-4(1H)- one | 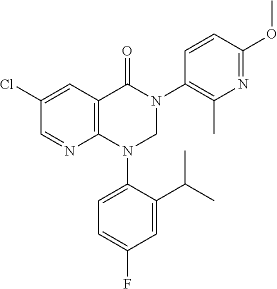 | MS (m/z) 413.0 (M + H)⁺ | 5-chloro-2-((4- fluoro-2- methylphenyl)a- mino)-N-(6- methoxy-2- methylpyridin-3- yl)nicotinamide |
| 718 | 1-(4-fluoro-2- isopropylphenyl)-3- (6-methoxy-2- methylpyridin-3-yl)- 7-(trifluoromethoxy)- 2,3- dihydroquinazolin- 4(1H)-one | 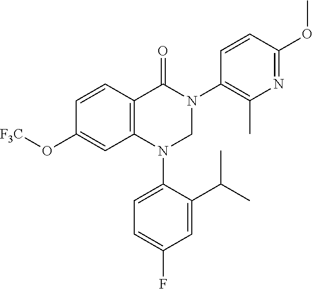 [SMILES: COc1ccc(N2CN(c3ccc(F)cc3C(C)C)c3cc(OC(F)(F)F)ccc3C2=O)c(C)n1] | MS (m/z) 490.0 (M + H)⁺ | 2-((4-fluoro-2- isopropylphenyl) amino)-N-(6- methoxy-2- methylpyridin-3- yl)-4- (trifluoromethoxy) benzamide |
| 719 | 6-chloro-7-fluoro-1- (4-fluoro-2- methylphenyl)-3-(6- methoxy-2- methylpyridin-3-yl)- 2,3- dihydroquinazolin- 4(1H)-one | 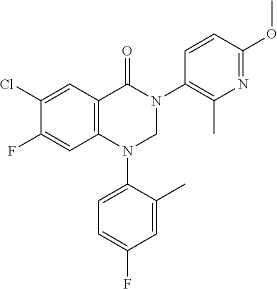 [SMILES: COc1ccc(N2CN(c3ccc(F)cc3C)c3cc(F)c(Cl)cc3C2=O)c(C)n1] | MS (m/z) 430.0 (M + H)⁺ | 5-chloro-4- fluoro-2-((4- fluoro-2- methylphenyl)a- mino)-N-(6- methoxy-2- methylpyridin-3- yl)benzamide |
| 720 | 6-chloro-3-(2-ethyl- 6-methoxypyridin-3- yl)-1-(4-fluoro-2- methylphenyl)-2,3- dihydroquinazolin- 4(1H)-one | 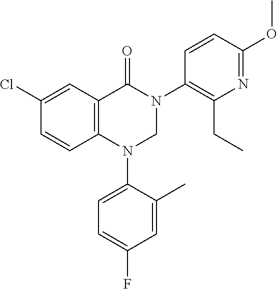 [SMILES: CCc1nc(OC)ccc1N1CN(c2ccc(F)cc2C)c2ccc(Cl)cc2C1=O] | MS (m/z) 426.1 (M + H)⁺ | 5-chloro-N-(2- ethyl-6- methoxypyridin- 3-yl)-2-((4- fluoro-2- methylphenyl)a- mino)benzamide |
| 721 | 7-fluoro-1-(4-fluoro- 2-methylphenyl)-3- (6-methoxy-2- methylpyridin-3-yl)- 6-(trifluoromethyl)- 2,3- dihydroquinazolin- 4(1H)-one | 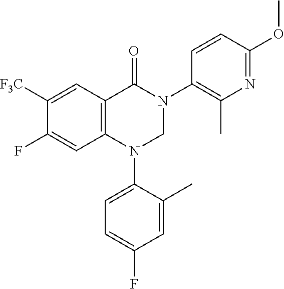 [SMILES: COc1ccc(N2CN(c3ccc(F)cc3C)c3cc(F)c(C(F)(F)F)cc3C2=O)c(C)n1] | MS (m/z) 464.2 (M + H)⁺ | 4-fluoro-2-((4- fluoro-2- methylphenyl)a- mino)-N-(6- methoxy-2- methylpyridin-3- yl)-5- (trifluoromethyl) benzamide |
| 722 | 5-fluoro-1-(4-fluoro- 2-methylphenyl)-3- (6-methoxy-2- methylpyridin-3-yl)- 6-(trifluoromethyl)- 2,3- dihydroquinazolin- 4(1H)-one | 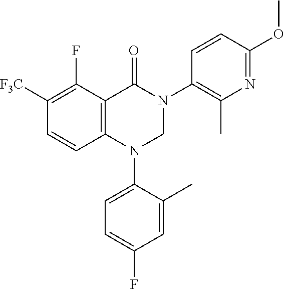 [SMILES: COc1ccc(N2CN(c3ccc(F)cc3C)c3ccc(C(F)(F)F)c(F)c3C2=O)c(C)n1] | MS (m/z) 464.0 (M + H)⁺ | 2-fluoro-6-((4- fluoro-2- methylphenyl)a- mino)-N-(6- methoxy-2- methylpyridin-3- yl)-3- (trifluoromethyl) benzamide |
| 723 | 6-fluoro-1-(4-fluoro- 2-methylphenyl)-3- (6-methoxy-2- methylpyridin-3-yl)- 7-(trifluoromethyl)- 2,3- dihydroquinazolin- 4(1H)-one | 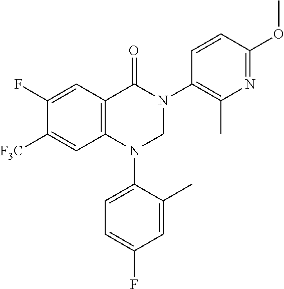 [SMILES: COc1ccc(N2CN(c3ccc(F)cc3C)c3cc(C(F)(F)F)c(F)cc3C2=O)c(C)n1] | MS (m/z) 464.0 (M + H)⁺ | 5-fluoro-2-((4- fluoro-2- methylphenyl)a- mino)-N-(6- methoxy-2- methylpyridin-3- yl)-4- (trifluoromethyl) benzamide |
| 724 | 6-bromo-7-fluoro-1- (4-fluoro-2- methylphenyl)-3-(6- methoxy-2- methylpyridin-3-yl)- 2,3- dihydroquinazolin- 4(1H)-one | 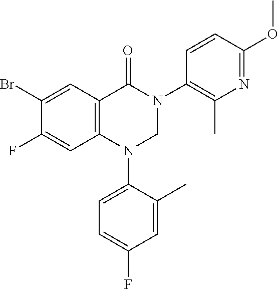 [SMILES: COc1ccc(N2CN(c3ccc(F)cc3C)c3cc(F)c(Br)cc3C2=O)c(C)n1] | MS (m/z) 474 (M + H)⁺ | 5-bromo-4- fluoro-2-((4- fluoro-2- methylphenyl)a- mino)-N-(6- methoxy-2- methylpyridin-3- yl)benzamide |
| 725 | 6-bromo-1-(4-fluoro- 2-methylphenyl)-3- (6-methoxy-2- methylpyridin-3-yl)- 7-(trifluoromethoxy)- 2,3- dihydroquinazolin- 4(1H)-one | 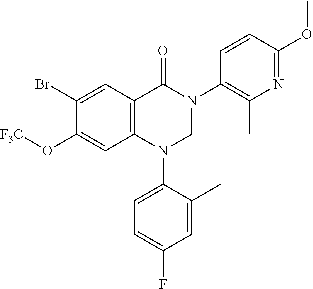 [SMILES: COc1ccc(N2CN(c3ccc(F)cc3C)c3cc(OC(F)(F)F)c(Br)cc3C2=O)c(C)n1] | MS (m/z) 540 (M + H)⁺ | 5-bromo-2-((4- fluoro-2- methylphenyl)a- mino)-N-(6- methoxy-2- methylpyridin-3- yl)-4- (trifluoromethoxy) benzamide |
| 726 | 1-(4,5-difluoro-2- methylphenyl)-3-(6- methoxy-2- methylpyridin-3-yl)- 6-(trifluoromethyl)- 2,3- dihydroquinazolin- 4(1H)-one | 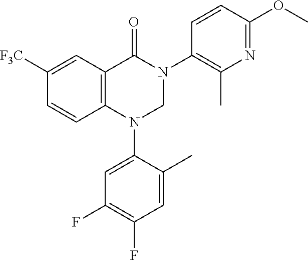 [SMILES: COc1ccc(N2CN(c3cc(F)c(F)cc3C)c3ccc(C(F)(F)F)cc3C2=O)c(C)n1] | MS (m/z) 464.2 (M + H)⁺ | 2-((4,5-difluoro- 2- methylphenyl)a- mino)-N-(6- methoxy-2- methylpyridin-3- yl)-5- (trifluoromethyl) benzamide |
| 727 | 1-(2-(tert-butyl)-4- fluorophenyl)-3-(6- methoxy-2- methylpyridin-3-yl)- 7-(trifluoromethyl)- 2,3- dihydroquinazolin- 4(1H)-one | 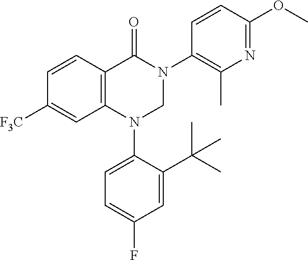 [SMILES: COc1ccc(N2CN(c3ccc(F)cc3C(C)(C)C)c3cc(C(F)(F)F)ccc3C2=O)c(C)n1] | MS (m/z) 488.2 (M + H)⁺ | 2-((2-(tert- butyl)-4- fluorophenyl)ami- no)-N-(6- methoxy-2- methylpyridin-3- yl)-4- (trifluoromethyl) benzamide |
| 728 | 1-(4-fluoro-2- methylphenyl)-3-(2- methoxy-4- methylpyrimidin-5- yl)-7- (trifluoromethyl)-2,3- dihydroquinazolin- 4(1H)-one | 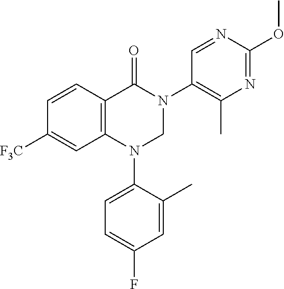 [SMILES: COc1ncc(N2CN(c3ccc(F)cc3C)c3cc(C(F)(F)F)ccc3C2=O)c(C)n1] | MS (m/z) 447.1 (M + H)⁺ | 2-((4-fluoro-2- methylphenyl)a- mino)-N-(2- methoxy-4- methylpyrimidin- 5-yl)-4- (trifluoromethyl) benzamide |
| 729 | 1-(2-ethyl-4- fluroophenyl)-6- fluoro-3-(6-methoxy- 2-methylpyridin-3- yl)-2,3- dihydroquinazolin- 4(1H)-one | 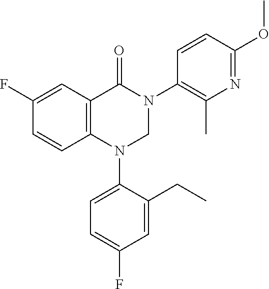 [SMILES: CCc1cc(F)ccc1N1CN(c2ccc(OC)nc2C)C(=O)c2cc(F)ccc21] | MS (m/z) 410.0 (M + H)⁺ | 2-((2-ethyl-4- fluorophenyl)ami- no)-5-fluoro-N- (6-methoxy-2- methylpyridin-3- yl)benzamide |
| 730 | 4-(6-chloro-3-(6- methoxy-2- methylpyridin-3-yl)- 4-oxo-3,4- dihydroquinazolin- 1(2H)-yl)-3- methylbenzonitrile | 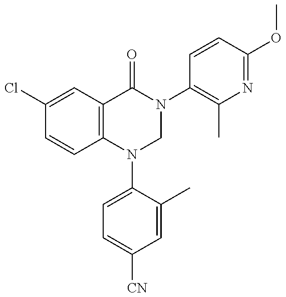 [SMILES: COc1ccc(N2CN(c3ccc(C#N)cc3C)c3ccc(Cl)cc3C2=O)c(C)n1] | MS (m/z) 419.0 (M + H)⁺ | 5-chloro-2-((4- cyano-2- methylphenyl)a- mino)-N-(6- methoxy-2- methylpyridin-3- yl)benzamide |
| 731 | 3-(6-methoxypyridin- 3-yl)-1-(2-methyl-4- (trifluoromethoxy)phen- yl)-6- (trifluoromethyl)-2,3- dihydropyrido[2,3- d]pyrimidin-4(1H)- one | 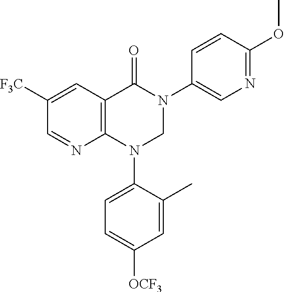 [SMILES: COc1ccc(N2CN(c3ccc(OC(F)(F)F)cc3C)c3ncc(C(F)(F)F)cc3C2=O)cn1] | MS (m/z) 498.8 (M + H)⁺ | N-(6- methoxypyridin- 3-yl)-2-((2- methyl-4- (trifluoromethoxy) phenyl)amino)- 5- (trifluoromethyl) nicotinamide |
| 732 | 1-(2,4-difluoro-6- methylphenyl)-3-(6- methoxy-2- mehtylpyridin-3-yl)- 6-(trifluoromethyl)- 2,3- dihydroquinazolin- 4(1H)-one | 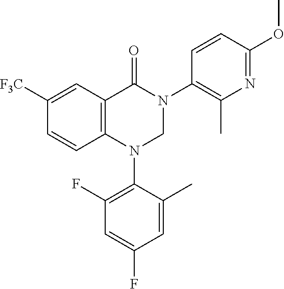 [SMILES: COc1ccc(N2CN(c3c(C)cc(F)cc3F)c3ccc(C(F)(F)F)cc3C2=O)c(C)n1] | MS (m/z) 464.0 (M + H)⁺ | 2-((2,4-difluoro- 6- methylphenyl)a- mino)-N-(6- methoxy-2- methylpyridin-3- yl)-5- (trifluoromethyl) benzamide |
| 733 | 7-(difluoromethyl)-1- (4-fluoro-2- isopropylphenyl)-3- (6-methoxy-2- methylpyridin-3-yl)- 2,3- dihydropyrido[2,3- d]pyrimidin-4(1H)- one | 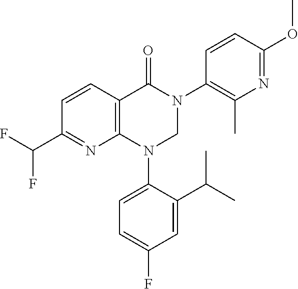 [SMILES: COc1ccc(N2CN(c3ccc(F)cc3C(C)C)c3nc(C(F)F)ccc3C2=O)c(C)n1] | MS (m/z) 457.1 (M + H)⁺ | 6- (difluoromethyl)- 2-((4-fluoro-2- isopropylphenyl) amino)-N-(6- methoxy-2- methylpyridin-3- yl)nicotinamide |
| 734 | 4-(3-(6-methoxy-2- methylpyridin-3-yl)- 4-oxo-6- (trifluoromethoxy)- 3,4- dihydroquinazolin- 1(2H)-yl)-3- methylbenzonitrile | 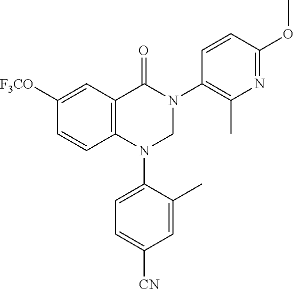 [SMILES: COc1ccc(N2CN(c3ccc(C#N)cc3C)c3ccc(OC(F)(F)F)cc3C2=O)c(C)n1] | MS (m/z) 469.1 (M + H)⁺ | 2-((4-cyano-2- methylphenyl)a- mino)-N-(6- methoxy-2- methylpyridin-3- yl)-5- (trifluoromethoxy) benzamide |
| 735 | 8-chloro-1-(4-fluoro- 2-methylphenyl)-3- (6-methoxy-2- methylpyridin-3-yl)- 2,3- dihydroquinazolin- 4(1H)-one | 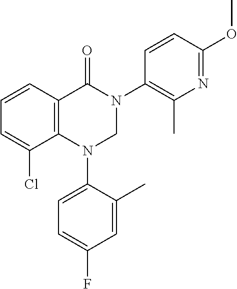 [SMILES: COc1ccc(N2CN(c3ccc(F)cc3C)c3c(Cl)cccc3C2=O)c(C)n1] | MS (m/z) 412.0 (M + H)⁺ | 3-chloro-2-((4- fluoro-2- methylphenyl)a- mino)-N-(6- methoxy-2- methylpyridin-3- yl)benzamide |
| 736 | 1-(4-methoxy-2- methylphenyl)-3-(6- methoxy-2- methylpyridin-3-yl)- 6-(trifluoromethyl)- 2,3- dihydroquinazolin- 4(1H)-one | 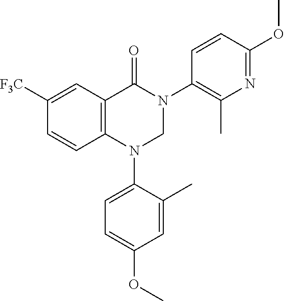 [SMILES: COc1ccc(N2CN(c3ccc(OC)nc3C)C(=O)c3cc(C(F)(F)F)ccc32)c(C)c1] | MS (m/z) 458.1 (M + H)⁺ | 2-((4-methoxy- 2- methylphenyl)a- mino)-N-(6- methoxy-2- methylpyridin-3- yl)-5- (trifluoromethyl) benzamide |
| 737 | 3-(6-chloro-3-(6- methoxy-2- methylpyridin-3-yl)- 4-oxo-3,4- dihydroquinazolin- 1(2H)-yl)-2- methylbenzonitrile | 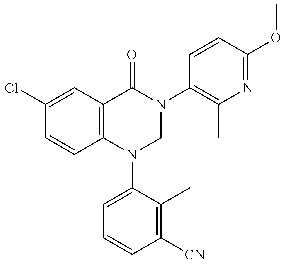 [SMILES: COc1ccc(N2CN(c3cccc(C#N)c3C)c3ccc(Cl)cc3C2=O)c(C)n1] | MS (m/z) 419.0 (M + H)⁺ | 5-chloro-2-((3- cyano-2- methylphenyl)a- mino)-N-(6- methoxy-2- methylpyridin-3- yl)benzamide |
| 738 | 6-fluoro-1-(4-fluoro- 2-methylphenyl)-3- (6-methoxy-2- methylpyridin-3-yl)- 7-(trifluoromethoxy)- 2,3- dihydroquinazolin- 4(1H)-one | 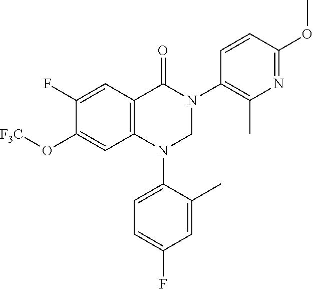 [SMILES: COc1ccc(N2CN(c3ccc(F)cc3C)c3cc(OC(F)(F)F)c(F)cc3C2=O)c(C)n1] | MS (m/z) 480.0 (M + H)⁺ | 5-fluoro-2-((4- fluoro-2- methylphenyl)a- mino)-N-(6- methoxy-2- methylpyridin-3- yl)-4- (trifluoromethoxy) benzamide |
| 739 | 3-(3-(6-methoxy-2- methylpyridin-3-yl)- 4-oxo-6- (trifluoromethyl)-3,4- dihydroquinazolin- 1(2H)-yl)-4- methylbenzonitrile | 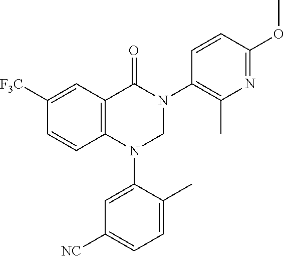 [SMILES: COc1ccc(N2CN(c3cc(C#N)ccc3C)c3ccc(C(F)(F)F)cc3C2=O)c(C)n1] | MS (m/z) 453.0 (M + H)⁺ | 2-((5-cyano-2- methylphenyl)a- mino)-N-(6- methoxy-2- methylpyridin-3- yl)-5- (trifluoromethyl) benzamide |
| 740 | 5-(difluoromethyl)-1- (4-fluoro-2- methylphenyl)-3-(6- methoxy-2- methylpyridin-3-yl)- 2,3- dihydroquinazolin- 4(1H)-one | 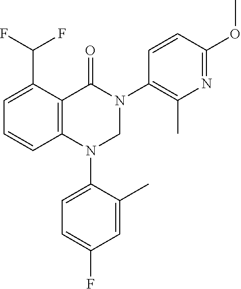 [SMILES: COc1ccc(N2CN(c3ccc(F)cc3C)c3cccc(C(F)F)c3C2=O)c(C)n1] | MS (m/z) 428.0 (M + H)⁺ | 2- (difluoromethyl)- 6-((4-fluoro-2- methylphenyl)a- mino)-N-(6- methoxy-2- methylpyridin-3- yl)benzamide |
| 741 | 1-(4-fluoro-2- methylphenyl)-3-(5- fluoro-6-methoxy-2- methylpyridin-3-yl)- 6-(trifluoromethyl)- 2,3- dihydroquinazolin- 4(1H)-one | 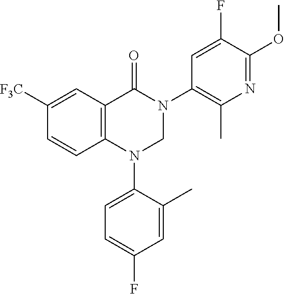 [SMILES: COc1nc(C)c(N2CN(c3ccc(F)cc3C)c3ccc(C(F)(F)F)cc3C2=O)cc1F] | MS (m/z) 463.8 (M + H)⁺ | 2-((4-fluoro-2- methylphenyl)a- mino)-N-(5- fluoro-6- methoxy-2- methylpyridin-3- yl)-5- (trifluoromethyl) benzamide |
| 742 | methyl 4-(6-chloro- 1-(4-fluoro-2- methylphenyl)-4- oxo-1,4- dihydroquinazolin- 3(2H)-yl)furan-2- carboxylate | 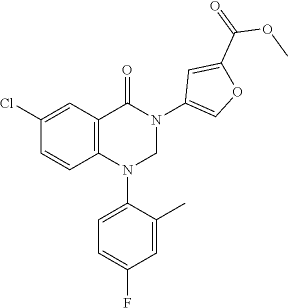 [SMILES: COC(=O)c1cc(N2CN(c3ccc(F)cc3C)c3ccc(Cl)cc3C2=O)co1] | MS (m/z) 415.0 (M + H)⁺ | methyl 4-(5- chloro-2-((4- fluoro-2- methylphenyl)a- mino)benzmido) furan-2- carboxylate |
| 743 | 6-chloro-5-fluoro-1- (4-fluoro-2- methylphenyl)-3-(2- methoxypyridin-4- yl)-2,3- dihydroquinazolin- 4(1H)-one | 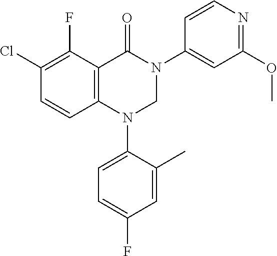 [SMILES: COc1cc(N2CN(c3ccc(F)cc3C)c3ccc(Cl)c(F)c3C2=O)ccn1] | MS (m/z) 416 (M + H)⁺ | 3-chloro-2- fluoro-6-((4- fluoro-2- methylphenyl)a- mino)-N-(2- methoxypyridin- 4-yl)benzamide |
| 744 | 6,7-difluoro-1-(4- fluoro-2- methoxyphenyl)-3- (6-methoxy-2- methylpyridin-3-yl)- 2,3- dihydroquinazolin- 4(1H)-one | 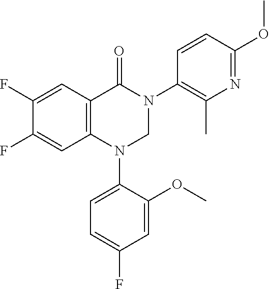 [SMILES: COc1ccc(N2CN(c3ccc(F)cc3OC)c3cc(F)c(F)cc3C2=O)c(C)n1] | MS (m/z) 429.8 (M + H)⁺ | 4,5-difluoro-2- ((4-fluoro-2- methoxyphenyl) amino)-N-(6- methoxy-2- methlpyridin-3- yl)benzamide |
| 745 | 6-chloro-3-(6- methoxy-2- methylpyridin-3-yl)- 1-(2-methoxy-4- methylphenyl)-2,3- dihydroquinazolin- 4(1H)-one | 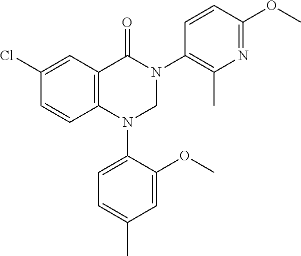 [SMILES: COc1ccc(N2CN(c3ccc(C)cc3OC)c3ccc(Cl)cc3C2=O)c(C)n1] | MS (m/z) 424 (M + H)⁺ | 5-chloro-N-(6- methoxy-2- methylpyridin-3- yl)-2-((2- methoxy-4- methylphenyl)a- mino)benzamide |
| 746 | 1-(2-fluoro-6- methylphenyl)-3-(6- methoxy-2- methylpyridin-3-yl)- 6-(trifluoromethyl)- 2,3- dihydroquinazolin- 4(1H)-one | 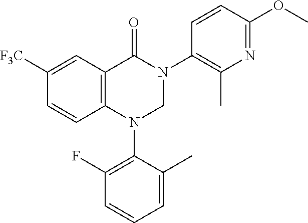 [SMILES: COc1ccc(N2CN(c3c(C)cccc3F)c3ccc(C(F)(F)F)cc3C2=O)c(C)n1] | MS (m/z) 446 (M + H)⁺ | 2-((2-fluoro-6- methylphenyl)a- mino)-N-(6- methoxy-2- methylpyridin-3- yl)-5- (trifluoromethyl) benzamide |
| 747 | 3-(3-bromophenyl)- 1-(4-fluoro-2- methylphenyl)-7- (trifluoromethyl)-2,3- dihydroquinazolin- 4(1H)-one | 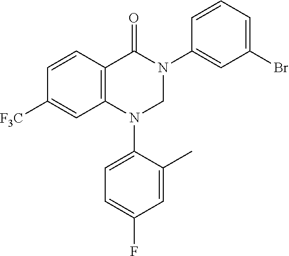 [SMILES: Cc1cc(F)ccc1N1CN(c2cccc(Br)c2)C(=O)c2ccc(C(F)(F)F)cc21] | MS (m/z) 479 (M + H)⁺ | N-(3- bromophenyl)- 2-((4-fluoro-2- methylphenyl)a- mino)-4- (trifluoromethyl) benzamide |
| 748 | 7-(difluoromethyl)-1- (4-fluoro-2- isopropylphenyl)-3- (6-methoxy-2- methylpyridin-3-yl)- 2,3- dihydroquinazolin- 4(1H)-one | 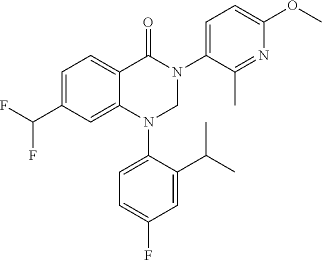 [SMILES: COc1ccc(N2CN(c3ccc(F)cc3C(C)C)c3cc(C(F)F)ccc3C2=O)c(C)n1] | MS (m/z) 456.1 (M + H)⁺ | 4- (difluoromethyl)- 2-((4-fluoro-2- isopropylphenyl) amino)-N-(6- methoxy-2- methylpyridin-3- yl)benzamide |
| 749 | 7-(difluoromethyl)-1- (4-fluoro-2- methylphenyl)-3-(6- methoxy-2- methylpyridin-3-yl)- 2,3- dihydroquinazolin- 4(1H)-one | 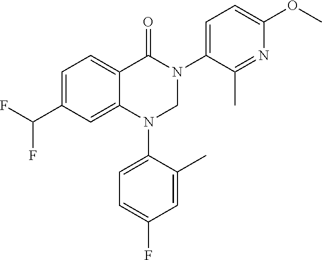 [SMILES: COc1ccc(N2CN(c3ccc(F)cc3C)c3cc(C(F)F)ccc3C2=O)c(C)n1] | MS (m/z) 428.0 (M + H)⁺ | 4- (difluoromethyl)- 2-((4-fluoro-2- methylphenyl)a- mino)-N-(6- methoxy-2- methylpyridin-3- yl)benzamide |
| 750 | 1- (bicyclo[1.1.1]pentan- 1-yl)-3-(6-methoxy- 2-methylpyridin-3- yl)-7- (trifluoromethyl)-2,3- dihydroquinazolin- 4(1H)-one | 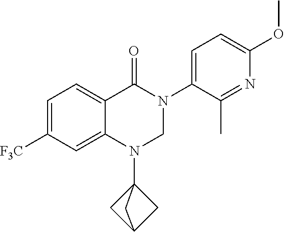 [SMILES: COc1ccc(N2CN(C34CC(C3)C4)c3cc(C(F)(F)F)ccc3C2=O)c(C)n1] | MS (m/z) 404.0 (M + H)⁺ | 2- (bicyclo[1.1.1]pen- tan-1- ylamino)-N-(6- methoxy-2- methylpyridin-3- yl)-4- (trifluoromethyl) benzamide |
| 751 | 6-chloro-3-(2-ethyl- 6-methoxypyridin-3- yl)-1-(4-fluoro-2- isopropylphenyl)- 2,3- dihydroquinazolin- 4(1H)-one | 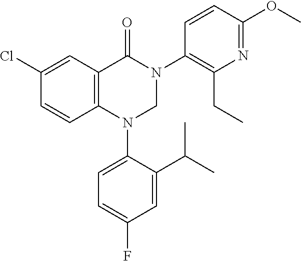 [SMILES: CCc1nc(OC)ccc1N1CN(c2ccc(F)cc2C(C)C)c2ccc(Cl)cc2C1=O] | MS (m/z) 454.0 (M + H)⁺ | 5-chloro-N-(2- ethyl-6- methoxypyridin- 3-yl)-2-((4- fluoro-2- isopropylphenyl) amino)benzamide |
| 752 | 6-fluoro-3-(3-(6- methoxy-2- methylpyridin-3-yl)- 4-oxo-6- (trifluoromethyl)-3,4- dihydroquinazolin- 1(2H)-yl)-2- methylbenzonitrile | 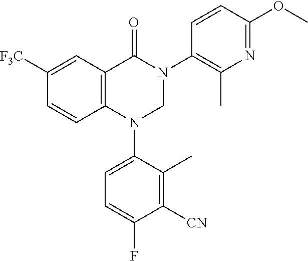 [SMILES: COc1ccc(N2CN(c3ccc(F)c(C#N)c3C)c3ccc(C(F)(F)F)cc3C2=O)c(C)n1] | MS (m/z) 471.0 (M + H)⁺ | 2-((3-cyano-4- fluoro-2- methylphenyl)a- mino)-N-(6- methoxy-2- methylpyridin-3- yl)-5- (trifluoromethyl) benzamide |
| 753 | 6-fluoro-1-(4-fluoro- 2-isopropylphenyl)- 3-(6-methoxy-2- methylpyridin-3-yl)- 2,3- dihydropyrido[2,3- d]pyrimidin-4(1H)- one | 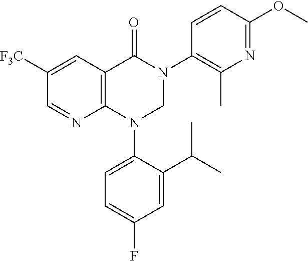 | MS (m/z) 425.2 (M + H)⁺ | 5-fluoro-2-((4- fluoro-2- isopropylphenyl) amino)-N-(6- methoxy-2- methylpyridin-3- yl)nicotinamide |
| 754 | 6-fluoro-1-(4-fluoro- 2-methylphenyl)-3- (6-methoxy-2- methylpyridin-3-yl)- 2,3- dihydroquinazolin- 4(1H)-one | 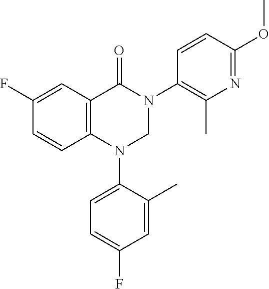 [SMILES: COc1ccc(N2CN(c3ccc(F)cc3C)c3ccc(F)cc3C2=O)c(C)n1] | MS (m/z) 396.1 (M + H)⁺ | 5-fluoro-2-((4- fluoro-2- methylphenyl)a- mino)-N-(6- methoxy-2- methylpyridin-3- yl)benzamide |
| 755 | 1-(4-fluoro-2- methylphenyl)-3-(2- methoxy-3- methylpyridin-4-yl)- 6-(trifluoromethyl)- 2,3- dihydroquinazolin- 4(1H)-one | 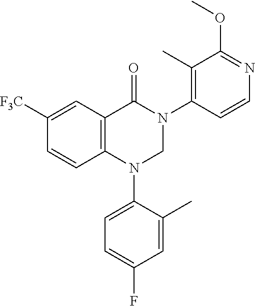 [SMILES: COc1nccc(N2CN(c3ccc(F)cc3C)c3ccc(C(F)(F)F)cc3C2=O)c1C] | MS (m/z) 446 (M + H)⁺ | 2-((4-fluoro-2- methylphenyl)a- mino)-N-(2- methoxy-3- methylpyridin-4- yl)-5- (trifluoromethyl) benzamide |

### Intermediate 756

### 1-(4-Fluorophenyl)-3-(6-methoxypyridin-3-yl)-7-(trifluoromethyl)-2,3-dihydroquinazolin-4(1H)-one

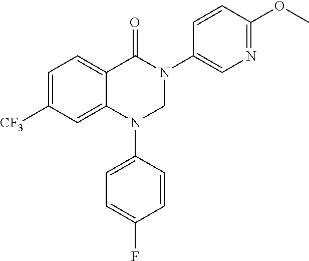 [SMILES: COc1ccc(N2CN(c3ccc(F)cc3)c3cc(C(F)(F)F)ccc3C2=O)cn1]

A mixture of 2-((4-fluorophenyl)amino)-N-(6-methoxypyridin-3-yl)-4-(trifluoromethyl)benzamide (280 mg, 0.691 mmol), paraformaldehyde (830 mg, 27.6 mmol) and PTSOH (131 mg, 0.691 mmol) in toluene (5.00 mL) was heated to reflux and stirred for 45 minutes. The reaction was cooled to RT, diluted with DCM and filtered. The filtrate was concentrated in vacuo and purified via column chromatography (Isco, 24 g column, 0-30% EtOAc/heptane) to give the title compound as a pale-yellow oil (156 mg, 0.374 mmol, 54.1% yield). MS (m/z) 418.3 (M+H)⁺.

Intermediates 757-778 were prepared from the indicated amide by methods analogous to those described for Intermediate 756. For Intermediate 757, acetaldehyde was used instead of paraformaldehyde.

| Int. | Name | Structure | Characterization | Amide |
|---|---|---|---|---|
| 757 | 1-(4-fluoro-2- methylphenyl)-3- (6- methoxypyridin- 3-yl)-2-methyl-7- (trifluoromethyl)- 2,3- dihydroquinazolin- 4(1H)-one | 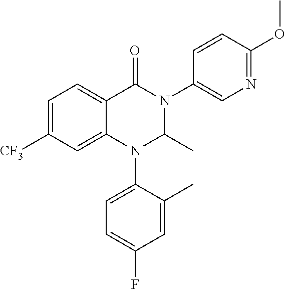 [SMILES: COc1ccc(N2C(=O)c3ccc(C(F)(F)F)cc3N(c3ccc(F)cc3C)C2C)cn1] | MS (m/z) 446.2 (M + H)⁺ | 2-((4-fluoro-2- methylphenyl) amino)-N-(6- methoxypyridin- 3-yl)-4- (trifluoromethyl) benzamide |
| 758 | 1-(4- fluorobenzyl)-3- (6- methoxypyridin- 3-yl)-7- (trifluoromethyl)- 2,3- dihydroquinazolin- 4(1H)-one | 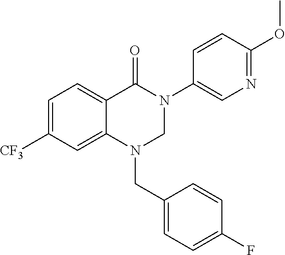 [SMILES: COc1ccc(N2CN(Cc3ccc(F)cc3)c3cc(C(F)(F)F)ccc3C2=O)cn1] | MS (m/z) 431.9 (M + H)⁺ | 2-((4- fluorobenzyl) amino)-N-(6- methoxypyridin- 3-yl)-4- (trifluoromethyl) benzamide |
| 759 | 1-(4-fluoro-2- methylphenyl)-3- (2- methoxypyridin- 4-yl)-7- (trifluoromethyl)- 2,3- dihydroquinazolin- 4(1H)-one | 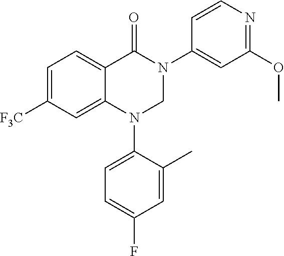 [SMILES: COc1cc(N2CN(c3ccc(F)cc3C)c3cc(C(F)(F)F)ccc3C2=O)ccn1] | MS (m/z) 432.3 (M + H)⁺ | 2-((4-fluoro-2- methylphenyl) amino)-N-(2- methoxypyridin- 4-yl)-4- (trifluoromethyl) benzamide |
| 760 | 3-(6-methoxy-2- methylpyridin-3- yl)-1-((1R,2S)-2- methylcyclohexyl)- 7- (trifluoromethyl)- 2,3- dihydroquinazolin- 4(1H)-one | 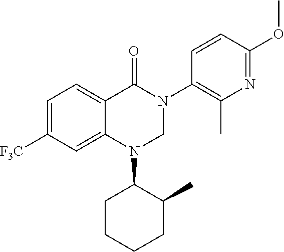 [SMILES: COc1ccc(N2CN([C@@H]3CCCC[C@@H]3C)c3cc(C(F)(F)F)ccc3C2=O)c(C)n1] | MS (m/z) 434.3 (M + H)⁺ | N-(6-methoxy- 2-methylpyridin- 3-yl)-2- (((1R,2S)-2- methylcyclohexyl) amino)-4- (trifluoromethyl) benzamide |
| 761 | 1-(4-fluoro-2- isopropylphenyl)- 3-(6-methoxy-2- methylpyridin-3- yl)-4-oxo- 1,2,3,4- tetrahydro- quinazoline-7- carbonitrile | 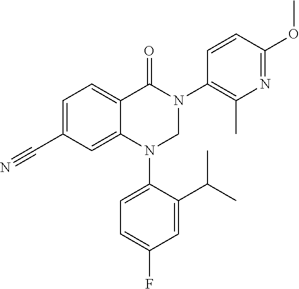 [SMILES: COc1ccc(N2CN(c3ccc(F)cc3C(C)C)c3cc(C#N)ccc3C2=O)c(C)n1] | MS (m/z) 431.3 (M + H)⁺ | 4-cyano-2-((4- fluoro-2- isopropylphenyl) amino)-N-(6- methoxy-2- methylpyridin-3- yl)benzamide |
| 762 | 3-(4-chloro-2- methoxypyrimidin- 5-yl)-1-(4- fluoro-2- methylphenyl)-6- (trifluoromethyl)- 2,3- dihydroquinazolin- 4(1H)-one | 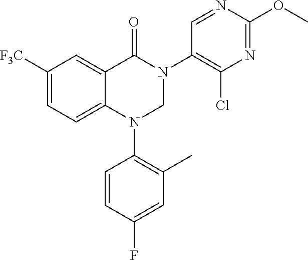 [SMILES: COc1ncc(N2CN(c3ccc(F)cc3C)c3ccc(C(F)(F)F)cc3C2=O)c(Cl)n1] | MS (m/z) 467.2 (M + H)⁺/469.2 (M + 3H)⁺ | N-(4-chloro-2- methoxypyrimidin- 5-yl)-2-((4- fluoro-2- methylphenyl) amino)-5- (trifluoromethyl) benzamide |
| 763 | 3-(6-methoxy-2- methylpyridin-3- yl)-1-(2-(2,2,2- trifluoroethyl) phenyl)-6- (trifluoromethyl)- 2,3- dihydroquinazolin- 4(1H)-one | 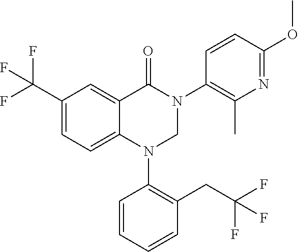 [SMILES: COc1ccc(N2CN(c3ccccc3CC(F)(F)F)c3ccc(C(F)(F)F)cc3C2=O)c(C)n1] | MS (m/z) 496 (M + H)⁺ | N-(6-methoxy- 2-methylpyridin- 3-yl)-2-((2- (2,2,2- trifluoroethyl) phenyl)amino)-5- (trifluoromethyl) benzamide |
| 764 | 1-(4-fluoro-2- methylphenyl)-3- (6-nitropyridin-3- yl)-7- (trifluoromethyl)- 2,3- dihydroquinazolin- 4(1H)-one | 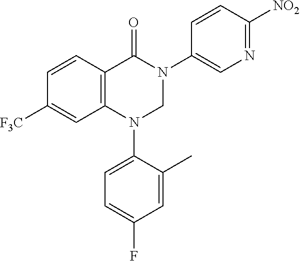 [SMILES: Cc1cc(F)ccc1N1CN(c2ccc([N+](=O)[O-])nc2)C(=O)c2ccc(C(F)(F)F)cc21] | MS (m/z) 447.2 (M + H)⁺ | 2-((4-fluoro-2- methylphenyl) amino)-N-(6- nitropyridin-3- yl)-4- (trifluoromethyl) benzamide |
| 765 | methyl 4-(1-(4- fluoro-2- methylphenyl)-4- oxo-7- (trifluoromethyl)- 1,4- dihydroquinazolin- 3(2H)- yl)benzoate | 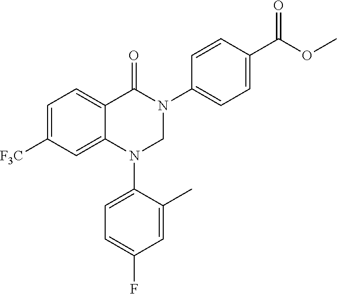 [SMILES: COC(=O)c1ccc(N2CN(c3ccc(F)cc3C)c3cc(C(F)(F)F)ccc3C2=O)cc1] | MS (m/z) 459.3 (M + H)⁺ | methyl 4-(2-((4- fluoro-2- methylphenyl) amino)-4- (trifluoromethyl) benzamido) benzoate |
| 766 | methyl 3-(1-(4- fluoro-2- methylphenyl)-4- oxo-7- (trifluoromethyl)- 1,4- dihydroquinazolin- 3(2H)- yl)benzoate | 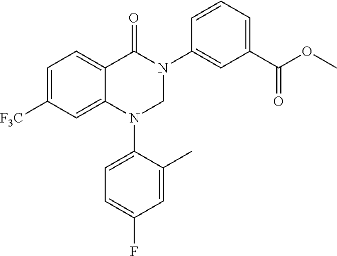 [SMILES: COC(=O)c1cccc(N2CN(c3ccc(F)cc3C)c3cc(C(F)(F)F)ccc3C2=O)c1] | MS (m/z) 459.2 (M + H)⁺ | methyl 3-(2-((4- fluoro-2- methylphenyl) amino)-4- (trifluoromethyl) benzamido) benzoate |
| 767 | 3-(4-((tert- butyldimethylsilyl) oxy)-2- methylphenyl)-1- (4-fluoro-2- methylphenyl)-6- (trifluoromethyl)- 2,3- dihydroquinazolin- 4(1H)-one | 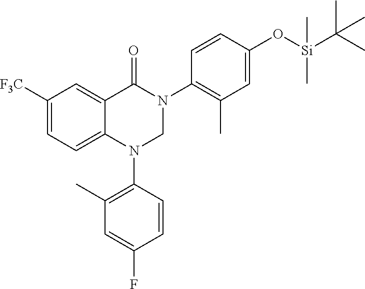 [SMILES: Cc1cc(O[Si](C)(C)C(C)(C)C)ccc1N1CN(c2ccc(F)cc2C)c2ccc(C(F)(F)F)cc2C1=O] | MS (m/z) 545.3 (M + H)⁺ | N-(4-((tert- butyldimethylsilyl) oxy)-2- methylphenyl)- 2-((4-fluoro-2- methylphenyl) amino)-5- (trifluoromethyl) benzamide |
| 768 | 3-(4-((tert- butyldimethylsilyl) oxy)-2- methylphenyl)-1- (4-fluoro-2- methylphenyl)-7- (trifluoromethyl)- 2,3- dihydroquinazolin- 4(1H)-one | 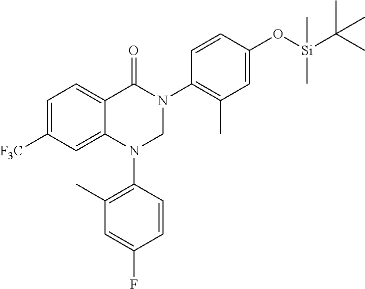 [SMILES: Cc1cc(O[Si](C)(C)C(C)(C)C)ccc1N1CN(c2ccc(F)cc2C)c2cc(C(F)(F)F)ccc2C1=O] | MS (m/z) 545.5 (M + H)⁺ | N-(4-((tert- butyldimethylsilyl) oxy)-2- methylphenyl)- 2-((4-fluoro-2- methylphenyl) amino)-4- (trifluoromethyl) benzamide |
| 769 | 1-((1S,3S)-3- fluorocyclopentyl)- 3-(6-methoxy- 2-methylpyridin- 3-yl)-7- (trifluoromethyl)- 2,3- dihydroquinazolin- 4(1H)-one 1-((1R,3R)-3- fluorocyclopentyl)- 3-(6-methoxy- 2-methylpyridin- 3-yl)-7- (trifluoromethyl)- 2,3- dihydroquinazolin- | 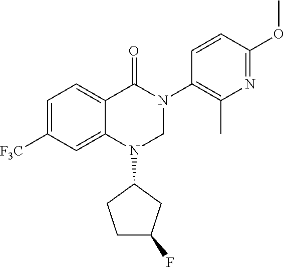 [SMILES: COc1ccc(N2CN([C@H]3CC[C@H](F)C3)c3cc(C(F)(F)F)ccc3C2=O)c(C)n1] | MS (m/z) 424.4 (M + H)⁺ | 2-((3- fluorocyclopentyl) amino)-N-(6- methoxy-2- methylpyridin-3- yl)-4- (trifluoromethyl) benzamide |
| | 4(1H)-one | | | |
| 770 | 3-(2-chloro-6- methoxypyridin- 3-yl)-1-(4-fluoro- 2-methylphenyl)- 6- (trifluoromethyl)- 2,3- dihydroquinazolin- 4(1H)-one | 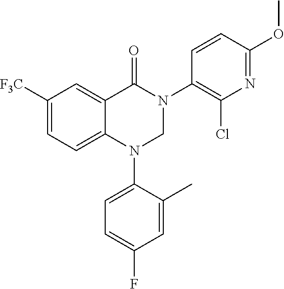 [SMILES: COc1ccc(N2CN(c3ccc(F)cc3C)c3ccc(C(F)(F)F)cc3C2=O)c(Cl)n1] | MS (m/z) 466.2 (M + H)⁺ | N-(2-chloro-6- methoxypyridin- 3-yl)-2-((4- fluoro-2- methylphenyl) amino)-5- (trifluoromethyl) benzamide |
| 771 | 1-benzyl-3-(6- methoxy-2- methylpyridin-3- yl)-6- (trifluoromethyl)- 2,3- dihydroquinazolin- 4(1H)-one | 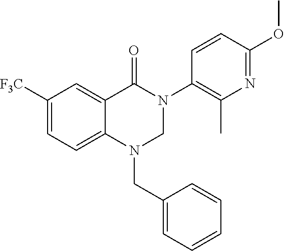 [SMILES: COc1ccc(N2CN(Cc3ccccc3)c3ccc(C(F)(F)F)cc3C2=O)c(C)n1] | MS (m/z) 428.0 (M + H)⁺ | 2- (benzylamino)- N-(6-methoxy- 2-methylpyridin- 3-yl)-5- (trifluoromethyl) benzamide |
| 772 | 1-benzyl-3-(6- methoxy-2- methylpyridin-3- yl)-7- (trifluoromethyl)- 2,3- dihydroquinazolin- 4(1H)-one | 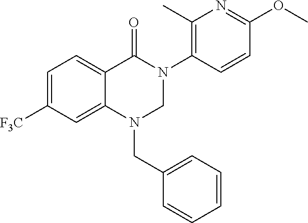 [SMILES: COc1ccc(N2CN(Cc3ccccc3)c3cc(C(F)(F)F)ccc3C2=O)c(C)n1] | MS (m/z) 428.2 (M + H)⁺ | 2- (benzylamino)- N-(6-methoxy- 2-methylpyridin- 3-yl)-4- (trifluoromethyl) benzamide |
| 773 | 3-(6- methoxypyridin- 3-yl)-1-(2- methyl-4- (trifluoromethoxy) phenyl)-6- (trifluoromethyl)- 2,3- dihydroquinazolin- 4(1H)-one | 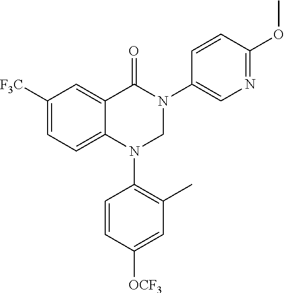 [SMILES: COc1ccc(N2CN(c3ccc(OC(F)(F)F)cc3C)c3ccc(C(F)(F)F)cc3C2=O)cn1] | MS (m/z) 498.0 (M + H)⁺ | N-(6- methoxypyridin- 3-yl)-2-((2- methyl-4- (trifluoromethoxy) phenyl)amino)- 5- (trifluoromethyl) benzamide |
| 774 | 7-chloro-1-(4- fluoro-2- methylphenyl)-3- (6-methoxy-2- methylpyridin-3- yl)-4-oxo- 1,2,3,4- tetrahydro- quinazoline-6- carbonitrile | 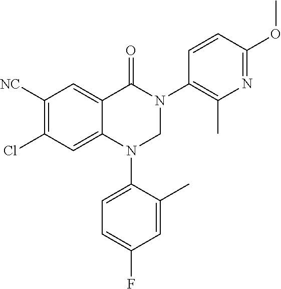 [SMILES: COc1ccc(N2CN(c3ccc(F)cc3C)c3cc(Cl)c(C#N)cc3C2=O)c(C)n1] | MS (m/z) 437.0 (M + H)⁺ | 4-chloro-5- cyano-2-((4- fluoro-2- methylphenyl) amino)-N-(6- methoxy-2- methylpyridin-3- yl)benzamide |
| 775 | 6-chloro-1-(4- fluoro-2- methylphenyl)-3- (6-methoxy-2- methylpyridin-3- yl)-5-methyl-2,3- dihydroquinazolin- 4(1H)-one | 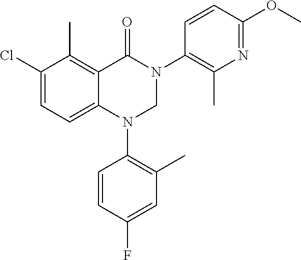 [SMILES: COc1ccc(N2CN(c3ccc(F)cc3C)c3ccc(Cl)c(C)c3C2=O)c(C)n1] | MS (m/z) 426.2 (M + H)⁺ | 3-chloro-6-((4- fluoro-2- methylphenyl) amino)-N-(6- methoxy-2- methylpyridin-3- yl)-2- methylbenzamide |
| 776 | 1-(3,5-difluoro-2- methylphenyl)-3- (6-methoxy-2- methylpyridin-3- yl)-6- (trifluoromethyl)- 2,3- dihydroquinazolin- 4(1H)-one | 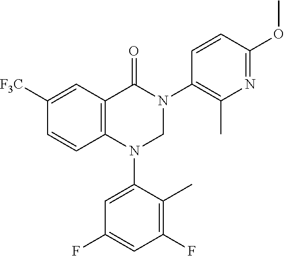 [SMILES: COc1ccc(N2CN(c3cc(F)cc(F)c3C)c3ccc(C(F)(F)F)cc3C2=O)c(C)n1] | MS (m/z) 464.0 (M + H)⁺ | 2-((3,5-difluoro- 2- methylphenyl) amino)-N-(6- methoxy-2- methylpyridin-3- yl)-5- (trifluoromethyl) benzamide |
| 777 | methyl 3-(6- chloro-1-(4- fluoro-2- methylphenyl)-4- oxo-1,4- dihydroquinazolin- 3(2H)-yl)furan- 2-carboxylate | 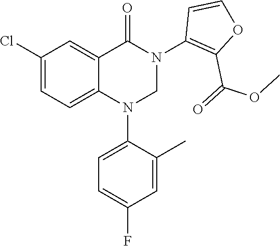 [SMILES: COC(=O)c1occc1N1CN(c2ccc(F)cc2C)c2ccc(Cl)cc2C1=O] | MS (m/z) 415.0 (M + H)⁺ | methyl 3-(5- chloro-2-((4- fluoro-2- methylphenyl) amino)benzamido) furan-2- carboxylate |
| 778 | 1-(4-fluoro-2- methylphenyl)-3- (6-methoxy-2- methylpyridin-3- yl)-6- (trifluoromethyl)- 1,2,3,4- tetrahydro- quinazoline | 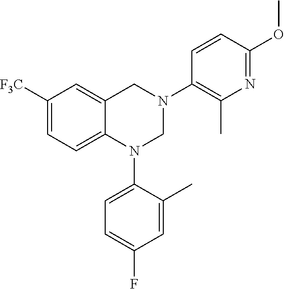 [SMILES: COc1ccc(N2Cc3cc(C(F)(F)F)ccc3N(c3ccc(F)cc3C)C2)c(C)n1] | MS (m/z) 432.0 (M + H)⁺ | N-(2-((4-fluoro- 2- methylphenyl) amino)-5- (trifluoromethyl) benzyl)-6- methoxy-2- methylpyridin-3- amine |

### Intermediate 779

### 1-(4-Fluoro-2-isopropylphenyl)-5-methoxy-3-(6-methoxy-2-methylpyridin-3-yl)-2,3-dihydroquinazolin-4(1H)-one

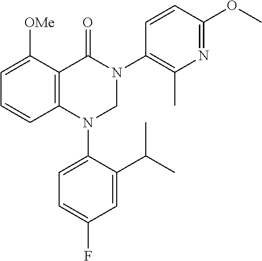 [SMILES: COc1ccc(N2CN(c3ccc(F)cc3C(C)C)c3cccc(OC)c3C2=O)c(C)n1]

To a stirring suspension of NaH (60% in oil) (0.189 g, 4.72 mmol) in DMF (3 mL) at 0° C., a solution of 2-((4-fluoro-2-isopropylphenyl)amino)-6-methoxy-N-(6-methoxy-2-methylpyridin-3-yl)benzamide (1 g, 2.361 mmol) in DMF (7 mL) was added. The reaction mixture was warmed to 30° C. and stirred for 30 min. The reaction was cooled to 0° C. and chloroiodomethane (0.514 mL, 7.08 mmol) was added dropwise. The resulting reaction mixture was allowed to warm to 30° C. and stirred for 3 hours. Upon completion, the reaction mixture was quenched with water (150 mL) and extracted with EtOAc (2×50 mL). The combined organic extracts were washed with water (25 mL) and brine (50 mL), dried over Na₂SO₄ and concentrated. The crude product was purified by column chromatography (Biotage, 25 g SNAP column, 0-38% EtOAc/petroleum ether over 60 min) to give the title product as a pale pink solid (530 mg, 1.169 mmol, 49.5% yield). MS (m/z) 436.2 (M+H)⁺.

### Intermediate 780

### Cis-rac-3-(6-methoxy-2-methylpyridin-3-yl)-1-((3R,4R)-3-methyltetrahydro-2H-pyran-4-yl)-7-(trifluoromethyl)-2,3-dihydroquinazolin-4(1H)-one

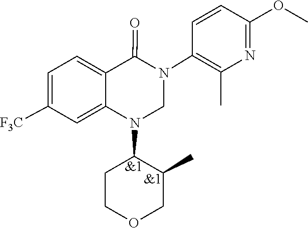 [SMILES: COc1ccc(N2CN([C@@H]3CCOC[C@@H]3C)c3cc(C(F)(F)F)ccc3C2=O)c(C)n1]

To a solution of cis-rac-N-(6-methoxy-2-methylpyridin-3-yl)-2-(((3R,4R)-3-methyltetrahydro-2H-pyran-4-yl)amino)-4-(trifluoromethyl)benzamide (0.390 g, 0.921 mmol) in chloroform (36.8 ml) was added paraformaldehyde (0.055 g, 1.842 mmol) followed by sulfuric acid (0.103 ml, 1.842 mmol) and heated to 60° C. for 1.5 hours. The reaction was cooled and the solvent was concentrated. The residue was suspended between EtOAc and water. The layers were separated and the organic layer was washed with water. The combined aqueous layers were washed with EtOAc. The combined organics were washed with water, brine and dried with MgSO₄ and the solvent was concentrated. The residue was purified via column chromatography (Isco, 40 g column, 0-50% EtOAc/heptane) to give the title compound (284 mg, 0.646 mmol, 70% yield). MS (m/z) 436 (M+H)⁺.

Intermediates 781-784 were prepared from the indicated amide by methods analogous to those described for Intermediate 780.

| Int. | Name | Structure | Characterization | Amide |
|---|---|---|---|---|
| 781 | 3-(2-bromo-6- methoxypyridin-3- yl)-1-(4-fluoro-2- methylphenyl)-6- (trifluoromethyl)- 2,3- dihydroquinazolin- 4(1H)-one | 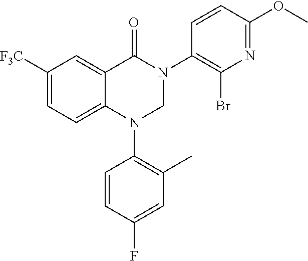 [SMILES: COc1ccc(N2CN(c3ccc(F)cc3C)c3ccc(C(F)(F)F)cc3C2=O)c(Br)n1] | MS (m/z) 510 (M + H)⁺/512 (M + 3H)⁺ | N-(2-bromo-6- methoxypyridin- 3-yl)-2-((4- fluoro-2- methylphenyl)a- mino)-5- (trifluoromethyl) benzamide |
| 782 | Trans-rac-3-(6- methoxy-2- methylpyridin-3-yl)- 1-((3R,4S)-3- methyltetrahydro- 2H-pyran-4-yl)-7- (trifluoromethyl)- 2,3- dihydroquinazolin- 4(1H)-one | 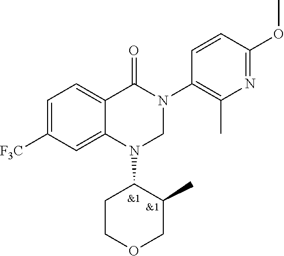 [SMILES: COc1ccc(N2CN([C@H]3CCOC[C@@H]3C)c3cc(C(F)(F)F)ccc3C2=O)c(C)n1] | MS (m/z) 436 (M + H)⁺ | trans-rac-N-(6- methoxy-2- methylpyridin-3- yl)-2-(((3R,4S)- 3- methyltetrahydro- 2H-pyran-4- yl)amino)-4- (trifluoromethyl) benzamide |
| 783 | 6-fluoro-1-(4-fluoro- 2-methylphenyl)-7- methoxy-3-(6- methoxy-2- methylpyridin-3-yl)- 2,3- dihydropyrido[2,3- d]pyrimidin-4(1H)- one | 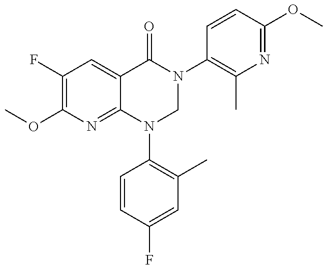 | MS (m/z) 427 (M + H)⁺ | 5-fluoro-2-((4- fluoro-2- methylphenyl)a- mino)-6- methoxy-N-(6- methoxy-2- methylpyridin-3- yl)nicotinamide |
| 784 | 1-(4-fluoro-2- methylphenyl)-3-(6- methoxy-4- methylpyridin-3-yl)- 6-(trifluoromethyl)- 2,3- dihydroquinazolin- 4(1H)-one | 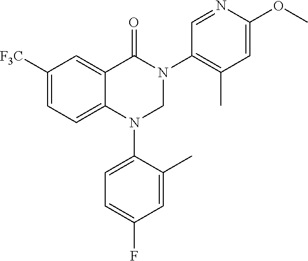 [SMILES: COc1cc(C)c(N2CN(c3ccc(F)cc3C)c3ccc(C(F)(F)F)cc3C2=O)cn1] | MS (m/z) 446.3 (M + H)⁺ | 2-((4-fluoro-2- methylphenyl)a- mino)-N-(6- methoxy-4- methylpyridin-3- yl)-5- (trifluoromethyl) benzamide |

### Intermediate 785

### 1-(4-Fluoro-2-methylphenyl)-3-(6-methoxypyridin-3-yl)-8-methyl-7-(trifluoromethyl)-2,3-dihydroquinazolin-4(1H)-one

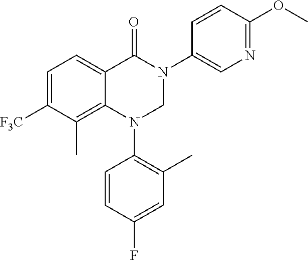 [SMILES: COc1ccc(N2CN(c3ccc(F)cc3C)c3c(ccc(C(F)(F)F)c3C)C2=O)cn1]

To a mixture of 3-chloro-2-((4-fluoro-2-methylphenyl)amino)-N-(6-methoxypyridin-3-yl)-4-(trifluoromethyl)benzamide (450 mg, 0.966 mmol), methylboronic acid (121 mg, 2.029 mmol) and tripotassium phosphate (1312 mg, 6.18 mmol) were added toluene (18 mL) and water (2 mL) and the reaction mixture was purged with N₂ for 5 minutes. Tricyclohexylphosphine tetrafluoroborate (49.7 mg, 0.135 mmol) and Pd(OAc)₂ (15.18 mg, 0.068 mmol) were then added and the resultant dark brown mixture was purged with N₂ for 5 minutes and then heated at 110° C. for 16 hours. The reaction was allowed to cool to RT and filtered through a pad of Celite washing with EtOAc (20 mL). The filtrate was dried over Na₂SO₄, filtered and concentrated in vacuo to a brown gum residue. The crude product was purified by column chromatography (Isolera, 25 g SNAP column, 30% EtOAc/petroleum ether). The obtained yellow solid was purified again by prep-HPLC (ymc, 19×250 mM, C18, 5 micron column, THF/MeCN) to give the title compound an off-white solid (250 mg, 0.531 mmol, 55.0% yield). MS (m/z) 446.0 (M+H)⁺.

Intermediates 786-788 were prepared from the indicated chloride by methods analogous to those described for Intermediate 785.

| Int. | Name | Structure | Characterization | Chloride |
|---|---|---|---|---|
| 786 | 1-(4-fluoro-2- methylphenyl)-3- (6-methoxy-2- methylpyridin-3- yl)-7-methyl-2,3- dihydroquinazolin- 4(1H)-one | 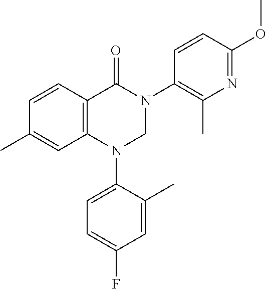 | MS (m/z) 392.2 (M + H)⁺ | 7-chloro-1-(4- fluoro-2- methylphenyl)-3- (6-methoxy-2- methylpyridin-3- yl)-2,3- dihydroquinazolin- 4(1H)-one |
| 787 | 1-(4-fluoro-2- methylphenyl)-3- (6-methoxy-2- methylpyridin-3- yl)-5-methyl-2,3- dihydroquinazolin- 4(1H)-one | 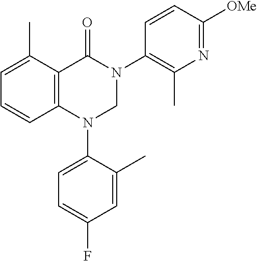 [SMILES: COc1ccc(N2CN(c3ccc(F)cc3C)c3cccc(C)c3C2=O)c(C)n1] | MS (m/z) 392.2 (M + H)⁺ | 5-chloro-1-(4- fluoro-2- methylphenyl)-3- (6-methoxy-2- methylpyridin-3- yl)-2,3- dihydroquinazolin- 4(1H)-one |
| 788 | 1-(4-fluoro-2- methylphenyl)-3- (6-methoxy-2- methylpyridin-3- yl)-8-methyl-2,3- dihydroquinazolin- 4(1H)-one | 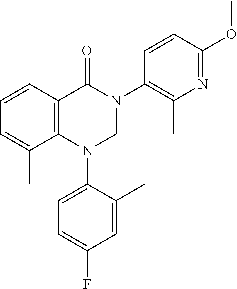 [SMILES: COc1ccc(N2CN(c3ccc(F)cc3C)c3c(C)cccc3C2=O)c(C)n1] | MS (m/z) 392.2 (M + H)⁺ | 8-chloro-1-(4- fluoro-2- methylphenyl)-3- (6-methoxy-2- methylpyridin-3- yl)-2,3- dihydroquinazolin- 4(1H)-one |

### Intermediate 789

### 4-(3-(6-Methoxy-2-methylpyridin-3-yl)-4-oxo-7-(trifluoromethyl)-3,4-dihydroquinazolin-1 (2H)-yl)-3-methylbenzonitrile

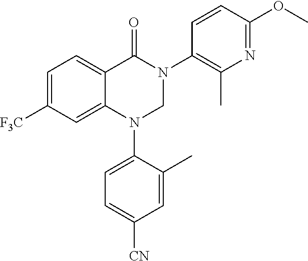 [SMILES: COc1ccc(N2CN(c3ccc(C#N)cc3C)c3cc(C(F)(F)F)ccc3C2=O)c(C)n1]

To a vial were added Pd(OAc)₂ (0.044 g, 0.198 mmol) and XPhos (0.188 g, 0.395 mmol) The vial was purged with N₂ for 10 minutes before sulfuric acid (50 mM in DMA) (3.95 ml, 0.198 mmol) was added. The mixture was purged with N₂ again and then heated to 80° C. under N₂ for 10 minutes to give a homogeneous coffee-brown solution. To another vial were added 1-(4-bromo-2-methylphenyl)-3-(6-methoxy-2-methylpyridin-3-yl)-7-(trifluoromethyl)-2,3-dihydroquinazolin-4(1H)-one (0.5 g, 0.988 mmol), dicyanozinc (0.1 g, 0.852 mmol), zinc (0.06 g, 0.918 mmol), and DMA (4.94 ml) and the reaction was purged with N₂ for 10 minutes before the catalyst solution prepared above was added. The reaction was purged with N₂ and then stirred at 120° C. for 20 minutes. The reaction was cooled to RT before water was added followed by brine. The reaction was filtered and the filtrate was extracted with EtOAc, dried over Na₂SO₄, filtered and concentrated under reduced pressure. The residue was purified by column chromatography (Isco, 24 g column, 0-40% EtOAc/heptane) to give the title compound as an off-white solid (292 mg, 0.645 mmol, 65.4% yield). MS (m/z) 453.3 (M+H)⁺.

Intermediates 790-791 were prepared from the indicated bromide by methods analogous to those described for Intermediate 789.

| Int. | Name | Structure | Characterization | Bromide |
|---|---|---|---|---|
| 790 | 1-(4-fluoro-2- methylphenyl)-3- (6-methoxy-2- methylpyridin-3- yl)-4-oxo-1,2,3,4- tetrahydroquinazo- line-7-carbonitrile | 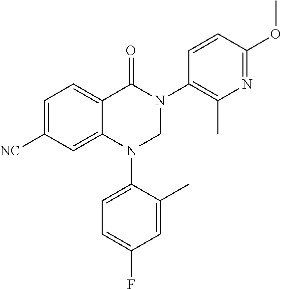 [SMILES: COc1ccc(N2CN(c3ccc(F)cc3C)c3cc(C#N)ccc3C2=O)c(C)n1] | MS (m/z) 403.3 (M + H)⁺ | 7-bromo-1-(4- fluoro-2- methylphenyl)-3- (6-methoxy-2- methylpyridin-3- yl)-2,3- dihydroquinazolin- 4(1H)-one |
| 791 | 3-(1-(4-fluoro-2- methylphenyl)-4- oxo-7- (trifluoromethyl)- 1,4- dihydroquinazolin- 3(2H)-yl)-6- methoxypicolinoni- trile | 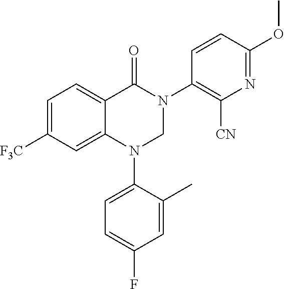 [SMILES: COc1ccc(N2CN(c3ccc(F)cc3C)c3cc(C(F)(F)F)ccc3C2=O)c(C#N)n1] | MS (m/z) 457.2 (M + H)⁺ | 3-(2-bromo-6- methoxypyridin-3- yl)-1-(4-fluoro-2- methylphenyl)-7- (trifluoromethyl)- 2,3- dihydroquinazolin- 4(1H)-one |

### Intermediate 792

### N-(3-(1-(4-Fluoro-2-methylphenyl)-4-oxo-6-(trifluoromethyl)-1,4-dihydroquinazolin-3(2H)-yl)-6-methoxypyridin-2-yl)acetamide

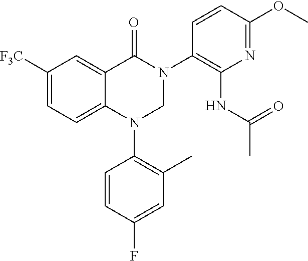 [SMILES: COc1ccc(N2CN(c3ccc(F)cc3C)c3ccc(C(F)(F)F)cc3C2=O)c(NC(C)=O)n1]

To a nitrogen flushed 20 ml vial were added acetamide (0.014 g, 0.235 mmol), cesium carbonate (0.089 g, 0.274 mmol), Pd₂(dba)₃ (8.97 mg, 9.80 μmol), and Xantphos (0.017 g, 0.029 mmol). In a separate nitrogen flushed vial was added 3-(2-bromo-6-methoxypyridin-3-yl)-1-(4-fluoro-2-methylphenyl)-6-(trifluoromethyl)-2,3-dihydroquinazolin-4(1H)-one (0.100 g, 0.196 mmol) and dissolved in 1,4-dioxane (2.450 ml). The dioxane solution was added to the mixture of components and heated to 95° C. for 4.5 hours. The reaction was cooled, diluted with DCM and filtered through Celite. The organic layer was washed with water (2×), brine, dried with MgSO₄ and concentrated under reduced pressure. The residue was purified by column chromatography (Isco, 24 g column, 0-40% EtOAc/DCM) to provide the title compound (71 mg, 0.145 mmol, 74% yield). MS (m/z) 489 (M+H)⁺.

Intermediate 793 was prepared from the indicated bromide by methods analogous to those described for Intermediate 792.

| Int. | Name | Structure | Characterization | Bromide |
|---|---|---|---|---|
| 793 | N-(5-(1-(4-fluoro- 2-methylphenyl)- 4-oxo-6- (trifluoromethyl)- 1,4- dihydroquinazolin- 3(2H)-yl)-6- methylpyridin-2- yl)acetamide | 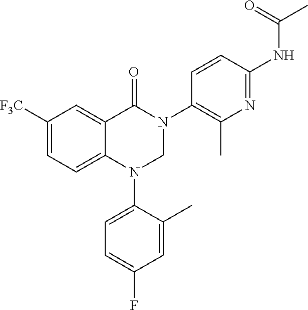 [SMILES: CC(=O)Nc1ccc(N2CN(c3ccc(F)cc3C)c3ccc(C(F)(F)F)cc3C2=O)c(C)n1] | MS (m/z) 473.3 (M + H)⁺ | 3-(6-bromo-2- methylpyridin-3- yl)-1-(4-fluoro-2- methylphenyl)-6- (trifluoromethyl)- 2,3- dihydroquinazolin- 4(1H)-one |

### Intermediate 794

### 3-(4-Amino-2-methoxypyrimidin-5-yl)-1-(4-fluoro-2-methylphenyl)-6-(trifluoromethyl)-2,3-dihydroquinazolin-(1H)-one

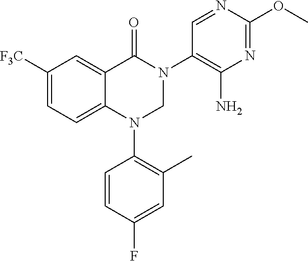 [SMILES: COc1ncc(N2CN(c3ccc(F)cc3C)c3ccc(C(F)(F)F)cc3C2=O)c(N)n1]

Ammonia (1 mL, 7.00 mmol) (7 M in MeOH) was added to 3-(4-chloro-2-methoxypyrimidin-5-yl)-1-(4-fluoro-2-methylphenyl)-6-(trifluoromethyl)-2,3-dihydroquinazolin-4(1H)-one (61 mg, 0.131 mmol) and the reaction mixture was heated at 110° C. for 30 min. After the reaction was cooled, solvent was removed and the crude material was moved to next step without further purification. MS (m/z) 448.3 (M+H)⁺.

### Intermediate 795

### 6-Chloro-3-(6-methoxy-2-methylpyridin-3-yl)-1-(2-methyl-4-(trifluoromethoxy)phenyl)-4-oxo-1,2,3,4-tetrahydroquinazoline-7-carbonitrile

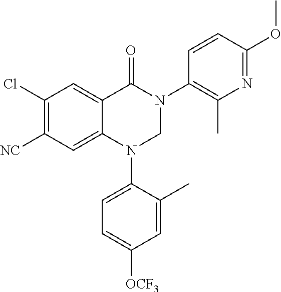 [SMILES: COc1ccc(N2CN(c3ccc(OC(F)(F)F)cc3C)c3cc(C#N)c(Cl)cc3C2=O)c(C)n1]

To a solution containing 6-chloro-7-fluoro-3-(6-methoxy-2-methylpyridin-3-yl)-1-(2-methyl-4-(trifluoromethoxy)phenyl)-2,3-dihydroquinazolin-4(1H)-one (0.85 g, 1.714 mmol) in DMSO (10.08 ml) were added sodium cyanide (0.101 g, 2.057 mmol) and tetrabutylammonium bromide (0.663 g, 2.057 mmol). The reaction mixture was warmed to 100° C. for 18 hr. The reaction was cooled and diluted with water, extracted with ethyl acetate, dried over MgSO₄, filtered and concentrated. The crude product was purified by column chromatography (Isco, 40 g column, 0-30% EtOAc/heptane) to afford the title compound as a yellow solid (830 mg, 1.650 mmol, 96% yield). MS (m/z) 503.1 (M+H)⁺.

Intermediates 796-799 were prepared from the indicated fluoride by methods analogous to those described for Intermediate 795.

| Int. | Name | Structure | Characterization | Fluoride |
|---|---|---|---|---|
| 796 | 6-chloro-1-(4- fluoro-2- methylphenyl)-3- (2-methoxy-4- methylpyrimidin- 5-yl)-4-oxo- 1,2,3,4- tetrahydroquinazo- line-7-carbonitrile | 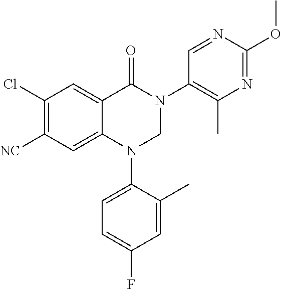 [SMILES: COc1ncc(N2CN(c3ccc(F)cc3C)c3cc(C#N)c(Cl)cc3C2=O)c(C)n1] | MS (m/z) 438.1 (M + H)⁺ | 6-chloro-7-fluoro- 1-(4-fluoro-2- methylphenyl)-3- (2-methoxy-4- methylpyrimidin- 5-yl)-2,3- dihydroquinazolin- 4(1H)-one |
| 797 | 6-chloro-1-(2- ethyl-4- fluorophenyl)-3- (6-methoxy-2- methylpyridin-3- yl)-4-oxo-1,2,3,4- tetrahydroquinazo- line-7-carbonitrile | 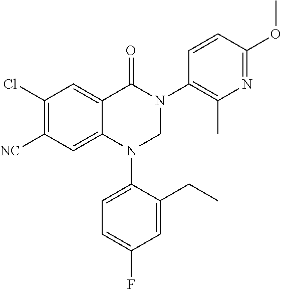 [SMILES: CCc1cc(F)ccc1N1CN(c2ccc(OC)nc2C)C(=O)c2cc(Cl)c(C#N)cc21] | MS (m/z) 451.3 (M + H)⁺ | 6-chloro-1-(2- ethyl-4- fluorophenyl)-7- fluoro-3-(6- methoxy-2- methylpyridin-3- yl)-2,3- dihydroquinazolin- 4(1H)-one |
| 798 | 6-chloro-1-(4- fluoro-2- methylphenyl)-3- (6-methoxy-2- methylpyridin-3- yl)-4-oxo-1,2,3,4- tetrahydroquinazo- line-7-carbonitrile | 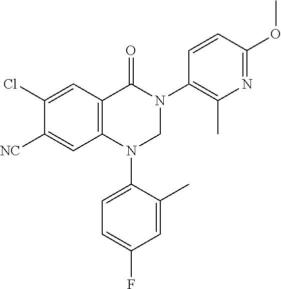 [SMILES: COc1ccc(N2CN(c3ccc(F)cc3C)c3cc(C#N)c(Cl)cc3C2=O)c(C)n1] | MS (m/z) 437.0 (M + H)⁺ | 6-chloro-7-fluoro- 1-(4-fluoro-2- methylphenyl)-3- (6-methoxy-2- methylpyridin-3- yl)-2,3- dihydroquinazolin- 4(1H)-one |
| 799 | 6-bromo-1-(4- fluoro-2- methylphenyl)-3- (6-methoxy-2- methylpyridin-3- yl)-4-oxo-1,2,3,4- tetrahydroquinazo- line-7-carbonitrile | 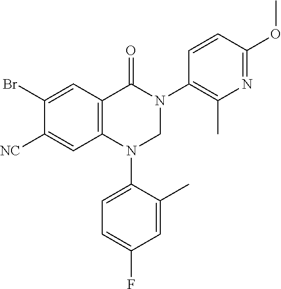 [SMILES: COc1ccc(N2CN(c3ccc(F)cc3C)c3cc(C#N)c(Br)cc3C2=O)c(C)n1] | MS (m/z) 481 (M + H)⁺//483 (M + 3H)⁺ | 6-bromo-7-fluoro- 1-(4-fluoro-2- methylphenyl)-3- (6-methoxy-2- methylpyridin-3- yl)-2,3- dihydroquinazolin- 4(1H)-one |

### Intermediate 800

### 1-(4-Fluoro-2-methylphenyl)-3-(6-methoxy-2-methylpyridin-3-yl)-4-oxo-7-(trifluoromethoxy)1,2,3,4-tetrahydroquinazoline-6-carbonitrile

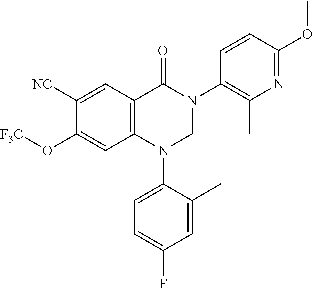 [SMILES: COc1ccc(N2CN(c3ccc(F)cc3C)c3cc(OC(F)(F)F)c(C#N)cc3C2=O)c(C)n1]

To a solution of 6-bromo-1-(4-fluoro-2-methylphenyl)-3-(6-methoxy-2-methylpyridin-3-yl)-7-(trifluoromethoxy)-2,3-dihydroquinazolin-4(1H)-one (270 mg, 0.500 mmol) in DMF (5 mL) stirred under nitrogen at RT, copper(I) cyanide (270 mg, 3.01 mmol) was added and the reaction mixture was stirred at 145° C. for 8 h. The reaction mixture was cooled to room temperature and filtered through a Celite pad washing with ethyl acetate (50 mL). Water (10 mL) was added to the filtrate and the mixture was stirred for 5 min. Layers were separated and the organic layer was concentrated under reduced pressure. The crude product was purified by reverse phase column chromatography (Grace reveleris ×2, 40 g C18 column, mobile phase A: 0.1% HCOOH in water, mobile phase B: acetonitrile, 0-67% of B/A over 0-40 min, 67% of B/A over 40-50 min) to give the title compound as an off-white solid (75 mg, 0.149 mmol, 29.9% yield). MS (m/z) 487.0 (M+H)⁺.

### Intermediate 801

### 1-(4-Fluoro-2-methylphenyl)-3-(6-methoxy-2-methylpyridin-3-yl)-4-oxo-6-(trifluoromethyl)-1,234-tetrahydroquinazoline-7-carbonitrile

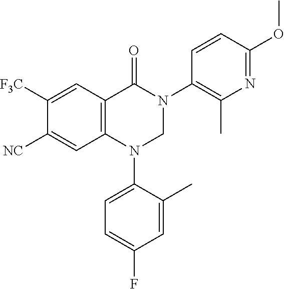 [SMILES: COc1ccc(N2CN(c3ccc(F)cc3C)c3cc(C#N)c(C(F)(F)F)cc3C2=O)c(C)n1]

To a stirred solution of 6-bromo-1-(4-fluoro-2-methylphenyl)-3-(6-methoxy-2-methylpyridin-3-yl)-4-oxo-1,2,3,4-tetrahydroquinazoline-7-carbonitrile (440 mg, 0.914 mmol) in DMF (3 mL), methyl 2,2-difluoro-2-(fluorosulfonyl)acetate (1581 mg, 8.23 mmol) and copper(I) iodide (157 mg, 0.823 mmol) were added at RT and the reaction mixture was heated at 90° C. for 16 hours. The reaction mixture was cooled, diluted with ethyl acetate (30 mL) and filtered through a Celite pad. The filtrate was washed with water (10 mL) and brine (10 mL), dried over Na₂SO₄, filtered and evaporated in vacuo. The crude product was purified by column chromatography (Isolera, 25 g SNAP column, 16-18% EtOAc/petroleum ether) to give the title compound as a yellow solid (125 mg, 0.218 mmol, 23.84% yield). MS (m/z) 471.0 (M+H)⁺.

### Intermediate 802

### 7-(1,1-Difluoro-2-hydroxyethyl)-1-(4-fluoro-2-methylphenyl)-3-(6-methoxy-2-methylpyridin-3-yl)-2,3-dihydroquinazolin-4(1H)-one

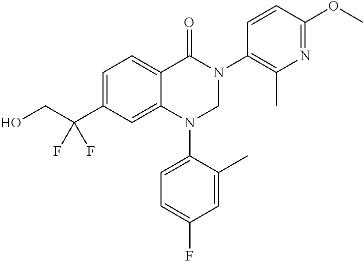 [SMILES: COc1ccc(N2CN(c3ccc(F)cc3C)c3cc(C(F)(F)CO)ccc3C2=O)c(C)n1]

### Step 1: Ethyl 2,2-difluoro-2-(1-(4-fluoro-2-methylphenyl)-3-(6-methoxy-2-methylpyridin-3-yl)-4-oxo-1,2,3,4-tetrahydroquinazolin-7-yl)acetate

A suspension of 7-bromo-1-(4-fluoro-2-methylphenyl)-3-(6-methoxy-2-methylpyridin-3-yl)-2,3-dihydroquinazolin-4(1H)-one (0.5 g, 1.096 mmol), [Pd(Pi-Cinnamyl)Cl]₂ (0.045 g, 0.087 mmol), zinc (0.215 g, 3.29 mmol), Xantphos (0.095 g, 0.164 mmol), and tetrabutylammonium bromide (0.530 g, 1.644 mmol) in THF (10.96 ml) in a sealed tube was purged with N₂ for 15 minutes before ethyl 2-bromo-2,2-difluoroacetate (0.422 ml, 3.29 mmol) was added. The tube was sealed and stirred at 70° C. for 2 days. The reaction was cooled, filtered, and concentrated. The residue was purified by column chromatography (Isco, 40 g column, 0-35% EtOAc/heptane) to give the title compound as a yellow foam (299 mg, 0.599 mmol, 54.6% yield). MS (m/z) 500.3 (M+H)⁺.

### Step 2: 7-(1,1-Difluoro-2-hydroxyethyl)-1-(4-fluoro-2-methylphenyl)-3-(6-methoxy-2-methylpyridin-3-yl)-2,3-dihydroquinazolin-4(1H)-one

Sodium borohydride (0.12 g, 3.17 mmol) was added portionwise to a suspension of ethyl 2,2-difluoro-2-(1-(4-fluoro-2-methylphenyl)-3-(6-methoxy-2-methylpyridin-3-yl)-4-oxo-1,2,3,4-tetrahydroquinazolin-7-yl)acetate (0.4 g, 0.801 mmol) in ethanol (2 ml) and methanol (2 ml) at 0° C. under N₂. The reaction was stirred at 0° C. for 5 minutes and then at RT for 1 hr. The reaction was cooled in an ice bath and quenched with sat. NH₄Cl (10 mL) and water (5 mL). Solvents were evaporated under reduced pressure. The solid precipitate was collected by filtration, washed with water and dried under vacuum to give the title compound as an off-white solid (300 mg, 0.656 mmol, 82% yield). MS (m/z) 458.3 (M+H)⁺.

### Intermediate 803

### 1-(4-(1,1-Difluoro-2-hydroxyethyl)-2-methylphenyl)-3-(6-methoxy-2-methylpyridin-3-yl)-7-(trifluoromethyl)-2,3-dihydroquinazolin-4(1H)-one

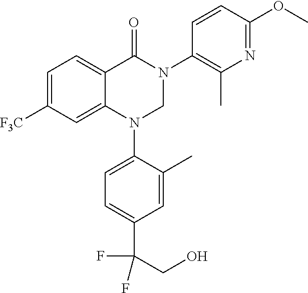 [SMILES: COc1ccc(N2CN(c3ccc(C(F)(F)CO)cc3C)c3cc(C(F)(F)F)ccc3C2=O)c(C)n1]

This intermediate was prepared from 1-(4-bromo-2-methylphenyl)-3-(6-methoxy-2-methylpyridin-3-yl)-7-(trifluoromethyl)-2,3-dihydroquinazolin-4(1H)-one by methods analogous to those described for Intermediate 802. MS (m/z) 508.2 (M+H)⁺.

### Intermediate 804

### 1-(4-Fluoro-2-methylphenyl)-3-(2-isopropyl-6-methoxypyridin-3-yl)-7-(trifluoromethyl)-2,3-dihydroquinazolin-4(1H)-one

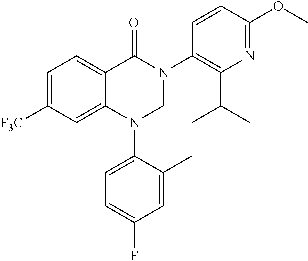 [SMILES: COc1ccc(N2CN(c3ccc(F)cc3C)c3cc(C(F)(F)F)ccc3C2=O)c(C(C)C)n1]

### Step 1: 1-(4-Fluoro-2-methylphenyl)-3-(6-methoxy-2-(prop-1-en-2-yl)pyridin-3-yl)-7-(trifluoromethyl)-2,3-dihydroquinazolin-4(1H)-one

A suspension of 3-(2-bromo-6-methoxypyridin-3-yl)-1-(4-fluoro-2-methylphenyl)-7-(trifluoromethyl)-2,3-dihydroquinazolin-4(1H)-one (0.2 g, 0.392 mmol), 4,4,5,5-tetramethyl-2-(prop-1-en-2-yl)-1,3,2-dioxaborolane (0.099 g, 0.588 mmol), sodium carbonate (0.125 g, 1.176 mmol) and PdCl₂(dppf-CH₂Cl₂ adduct (0.032 g, 0.039 mmol) was purged with N₂ before 1,4-dioxane (2.488 ml) and water (0.124 ml) were added. The reaction mixture was purged with N₂ for 10 minutes, sealed and stirred at 80° C. overnight. After cooled down, the reaction was filtered and concentrated. The residue was dissolved in DCM (˜5 mL) and purified by column chromatography (Isco, 24 g column, 0-25% EtOAc/heptane) to give the title compound as an off-white foam (73 mg, 0.155 mmol, 39.5% yield). MS (m/z) 472.4 (M+H)⁺.

### Step 2: 1-(4-Fluoro-2-methylphenyl)-3-(2-isopropyl-6-methoxypyridin-3-yl)-7-(trifluoromethyl)-2,3-dihydroquinazolin-4(1H)-one

Pd/C (0.016 g, 0.015 mmol) was added to a flask contained 1-(4-fluoro-2-methylphenyl)-3-(6-methoxy-2-(prop-1-en-2-yl)pyridin-3-yl)-7-(trifluoromethyl)-2,3-dihydroquinazolin-4(1H)-one (0.07 g, 0.148 mmol) under N₂. ethanol (5 ml) was added and the reaction mixture was purged with N₂ for 10 minutes. The reaction was hydrogenated for 1 hr using a hydrogen balloon. The reaction was purged with N₂ and then filtered through a pad of Celite. The filtrate was concentrated to give the title compound as a white solid (65 mg, 0.137 mmol, 92% yield). MS (m/z) 474.3 (M+H)⁺.

### Intermediate 805

### 3-(2-Cyclopropyl-6-methoxypyridin-3-yl)-1-(4-fluoro-2-methylphenyl)-7-(trifluoromethyl)-2,3-dihydroquinazolin-4(1H)-one

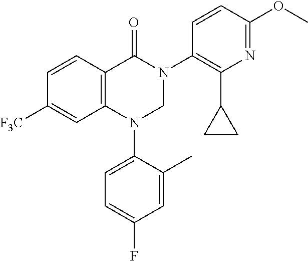 [SMILES: COc1ccc(N2CN(c3ccc(F)cc3C)c3cc(C(F)(F)F)ccc3C2=O)c(C2CC2)n1]

A 50 mL round bottom flask equipped with a magnetic stir bar and nitrogen inlet was charged with 3-(2-bromo-6-methoxypyridin-3-yl)-1-(4-fluoro-2-methylphenyl)-7-(trifluoromethyl)-2,3-dihydroquinazolin-4(1H)-one (0.35 g, 0.686 mmol), 1,4-dioxane (10 mL), potassium carbonate (0.114 g, 0.823 mmol), water (2 mL), and cyclopropylboronic acid (0.071 g, 0.823 mmol). To this mixture was added PdCl₂(dppf-CH₂Cl₂ adduct (0.028 g, 0.034 mmol). The flask was purged with nitrogen for 5 minutes and then heated to 80° C. for 12 h. The reaction mixture was cooled to RT and filtered through a Celite pad and the filtrate was concentrated under vacuum. The crude product was purified by column chromatography (Isolera, 100 g SNAP column, EtOAc/petroleum ether, 0-30% over 80 min) to give the title compound as a yellow liquid (0.2 g, 0.399 mmol, 58.1% yield). MS (m/z) 472.0 (M+H)⁺.

### Intermediate 806

### 1-(4-Fluoro-2-methylphenyl)-3-(2-((2-hydroxyethyl)amino)-6-methoxypyridin-3-yl)-7-(trifluoromethyl)-2,3-dihydroquinazolin-4(1H)-one

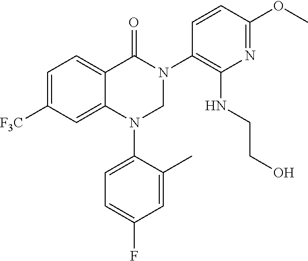 [SMILES: COc1ccc(N2CN(c3ccc(F)cc3C)c3cc(C(F)(F)F)ccc3C2=O)c(NCCO)n1]

In a microwave vial, a suspension of 3-(2-bromo-6-methoxypyridin-3-yl)-1-(4-fluoro-2-methylphenyl)-7-(trifluoromethyl)-2,3-dihydroquinazolin-4(1H)-one (0.3 g, 0.588 mmol), 2-aminoethan-1-ol (0.142 ml, 2.352 mmol), BINAP (0.037 g, 0.059 mmol), tris(dibenzylideneacetone)dipalladium(0) (0.027 g, 0.029 mmol) and cesium carbonate (0.287 g, 0.882 mmol) in toluene (5 ml) was purged with N₂ for 15 minutes. The vial was sealed and stirred at 100° C. overnight. The reaction was cooled, concentrated and purified by column chromatography (Isco, 24 g column, 0-50% EtOAc/heptane) to give the title compound as a tan solid (221 mg, 0.451 mmol, 77% yield). MS (m/z) 491.3 (M+H)⁺.

### Intermediate 807

### 6-Chloro-1-(4-fluoro-2-(methylamino)phenyl)-3-(6-methoxy-2-methylpyridin-3-yl)-2,3-dihydroquinazolin-4(1H)-one

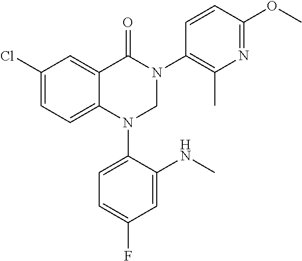 [SMILES: CNc1cc(F)ccc1N1CN(c2ccc(OC)nc2C)C(=O)c2cc(Cl)ccc21]

A suspension of 1-(2-bromo-4-fluorophenyl)-6-chloro-3-(6-methoxy-2-methylpyridin-3-yl)-2,3-dihydroquinazolin-4(1H)-one (0.5 g, 1.049 mmol), methanamine (3.15 ml, 6.29 mmol), BINAP (0.065 g, 0.105 mmol), tris(dibenzylideneacetone)dipalladium(0) (0.048 g, 0.052 mmol) and cesium carbonate (0.513 g, 1.573 mmol) in 1,4-dioxane (5 ml) in a microwave vial was purged with N₂ for 15 minutes. The vial was sealed and then stirred at 100° C. for 18 hr. After cooled down, the reaction was concentrated and purified by column chromatography (Isco Combiflash Rf, 24 g column, 0-100% EtOAc/heptane over 30 min). The product obtained was purified again using similar condition to give the title product as a tan foam (127 mg, 0.298 mmol, 28.4% yield). MS (m/z) 427.3 (M+H)⁺.

### Intermediate 808

### 6-Chloro-1-(2-(dimethylamino)-4-fluorophenyl)-3-(6-methoxy-2-methylpyridin-3-yl)-2,3-dihydroquinazolin-4(1H)-one

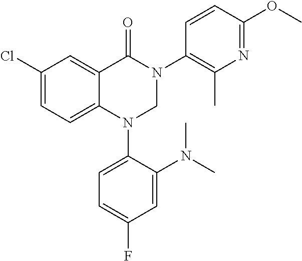 [SMILES: COc1ccc(N2CN(c3ccc(F)cc3N(C)C)c3ccc(Cl)cc3C2=O)c(C)n1]

Sodium hydride (0.019 g, 0.478 mmol) was added to a solution of 6-chloro-1-(4-fluoro-2-(methylamino)phenyl)-3-(6-methoxy-2-methylpyridin-3-yl)-2,3-dihydroquinazolin-4(1H)-one (0.034 g, 0.080 mmol) in DMF (0.675 ml) under N₂ and the reaction was stirred for 10 minutes. methyl iodide (0.050 ml, 0.796 mmol) was added and the reaction mixture was stirred for 2.5 hr. The reaction was cooled in an ice bath, quenched with sat. NH₄Cl, extracted with EtOAc, dried over Na₂SO₄ and concentrated. The residue was purified by column chromatography (Isco, 12 g column, 0-100% EtOAc/heptane) to give title compound as a sticky solid (61 mg, 0.138 mmol, 66.8% yield). MS (m/z) 441.3 (M+H)⁺.

### Intermediate 809

### 1-(2,4-Dimethylphenyl)-3-(6-methoxy-2-methylpyridin-3-yl)-7-(trifluoromethyl)-2,3-dihydroquinazolin-4(1H)-one

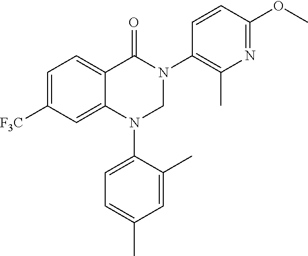 [SMILES: COc1ccc(N2CN(c3ccc(C)cc3C)c3cc(C(F)(F)F)ccc3C2=O)c(C)n1]

In a microwave vial, a suspension of 1-(4-bromo-2-methylphenyl)-3-(6-methoxy-2-methylpyridin-3-yl)-7-(trifluoromethyl)-2,3-dihydroquinazolin-4(1H)-one (0.16 g, 0.316 mmol), 2,4,6-trimethyl-1,3,5,2,4,6-trioxatriborinane (0.048 g, 0.379 mmol), cesium carbonate (0.175 g, 0.537 mmol), and PdCl₂(dppf-CH₂Cl₂ adduct (0.026 g, 0.032 mmol) in 1,4-dioxane (1.5 ml) and water (0.214 ml) was purged with N₂ for 20 minutes. The reaction vial was sealed and heated at 110° C. overnight. The reaction was cooled and filtered. Solvent was removed and the residue was partitioned between EtOAc and water. The organic layer was dried over Na₂SO₄ and concentrated. The residue was purified by column chromatography (Isco, 12 g column, 0-20% EtOAc/heptane over 17 minutes) to give the title compound as an off-white solid (79 mg, 0.179 mmol, 56.6% yield). MS (m/z) 442.3 (M+H)⁺.

### Intermediate 810

### 1-(4-Fluoro-2-methylphenyl)-3-(6-methoxy-2-methylpyridin-3-yl)-7-(methylamino)-2,3-dihydroquinazolin-4(1H)-one

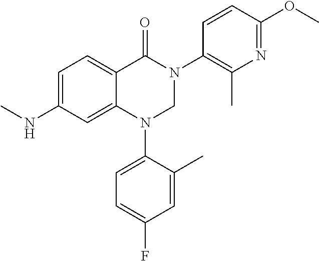

A solution of 7-chloro-1-(4-fluoro-2-methylphenyl)-3-(6-methoxy-2-methylpyridin-3-yl)-2,3-dihydroquinazolin-4(1H)-one (600 mg, 1.457 mmol), cesium carbonate (2136 mg, 6.56 mmol), (t-Bu)PhCPhos precatalyst (226 mg, 0.291 mmol) in 1,4-dioxane (20 mL) was purged with nitrogen for 15 min in a tensile sealed tube before Pd₂(dba)₃ (133 mg, 0.146 mmol) was added followed by methylamine hydrochloride (197 mg, 2.91 mmol) under nitrogen. The resulting reaction mixture was stirred at 110° C. for 48 h. The reaction mixture was filtered through a Celite bed washing with EtOAc and concentrated under reduced pressure. The crude product was purified by column chromatography (Isolera, 50 g column, 0-100% EtOAc/petroleum ether) to give the title compound as a pale yellow solid (0.2 g, 51.7% pure, 0.254 mmol, 17.5% yield). MS (m/z) 407.2 (M+H)⁺.

### Intermediate 811

### 3-(6-Methoxy-2-methylpyridin-3-yl)-6-(trifluoromethyl)-2,3-dihydroquinazolin-4(1H)-one

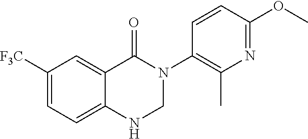 [SMILES: COc1ccc(N2CNc3ccc(C(F)(F)F)cc3C2=O)c(C)n1]

A solution of 1-benzyl-3-(6-methoxy-2-methylpyridin-3-yl)-6-(trifluoromethyl)-2,3-dihydroquinazolin-4(1H)-one (730 mg, 1.708 mmol) in methanol (15 mL) was hydrogenated under 1 atm (balloon) pressure over Pd—C (10% wt) (182 mg, 0.171 mmol) at room temperature for 16 hours. The reaction mixture was purged with nitrogen for 5 minutes and Pd—C (10% wt) (145 mg, 0.137 mmol) was once again added to the reaction mixture. The resulting reaction mixture was hydrogenated under 1 atm (balloon) pressure at room temperature for an additional 6 hours. The reaction mixture was filtered through a Celite pad and the Celite pad was washed with EtOAc (100 mL). The filtrate was concentrated in vacuo. The residue was purified by column chromatography (Isolera, 25 g column, 0-25% EtOAc/petroleum ether) to give the title compound as a white solid (320 mg, 0.915 mmol, 53.5% yield). MS (m/z) 338.2 (M+H)⁺.

Intermediate 812 was prepared from the indicated benzyl by method analogous to those described for intermediate 811.

| Int. | Name | Structure | Characterization | Benzyl |
|---|---|---|---|---|
| 812 | 3-(6-methoxy-2- methylpyridin-3- yl)-7- (trifluoromethyl)- 2,3- dihydroquinazolin- 4(1H)-one | 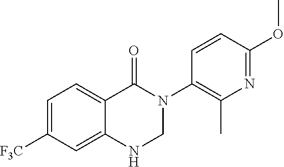 [SMILES: COc1ccc(N2CNc3cc(C(F)(F)F)ccc3C2=O)c(C)n1] | MS (m/z) 338.1 (M + H)⁺ | 1-benzy1-3-(6- methoxy-2- methylpyridin-3- yl)-7- (trifluoromethyl)- 2,3- dihydroquinazolin- 4(1H)-one |

### Intermediate 813

### 3-(6-Methoxy-2-methylpyridin-3-yl)-1-phenyl-7-(trifluoromethyl)-2,3-dihydroquinazolin-4(1H)-one

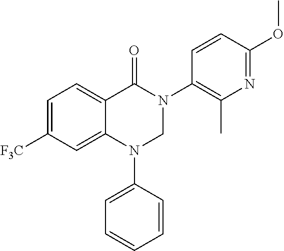 [SMILES: COc1ccc(N2CN(c3ccccc3)c3cc(C(F)(F)F)ccc3C2=O)c(C)n1]

To a solution of 3-(6-methoxy-2-methylpyridin-3-yl)-7-(trifluoromethyl)-2,3-dihydroquinazolin-4(1H)-one (0.5 g, 1.482 mmol) and bromobenzene (0.349 g, 2.224 mmol) in 1,4-dioxane (5 mL) stirred under N₂ at RT were added cesium carbonate (0.966 g, 2.96 mmol) and 2,2′-bis(diphenylphosphaneyl)-1,1′-binaphthalene (0.092 g, 0.148 mmol). The reaction mixture was purged with N₂ for 10 min before Pd₂(dba)₃ (0.068 g, 0.074 mmol) was added. After stirring at 100° C. for 16 hr, the reaction was cooled to RT, filtered through Celite and concentrated. The crude product was purified by column chromatography (Isolera, 100 g SNAP column, 0-30% EtOAc/petroleum ether over 80 mins) to give the title compound as a yellow solid (500 mg, 0.798 mmol, 53.9% yield). MS (m/z) 414.0 (M+H)⁺.

Intermediates 814-816 were prepared from the indicated aryl bromide and aniline by methods analogous to those described for Intermediate 813. For intermediate 816, BrettPhos-Pd-G3 was used instead of 2,2′-bis(diphenylphosphaneyl)-1,1′-binaphthalene and Pd₂(dba)₃. Additionally, sodium tert-butoxide was used instead of cesium carbonate.

| | | | | Aryl | |
|---|---|---|---|---|---|
| Int. | Name | Structure | Characterization | Bromide | Aniline |
| 814 | 3-(6-methoxy- 2- methylpyridin- 3-yl)-1-(o-tolyl)- 7- (trifluoromethyl)- 2,3- dihydroquinazo- lin-4(1H)-one | 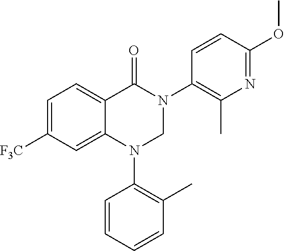 [SMILES: COc1ccc(N2CN(c3ccccc3C)c3cc(C(F)(F)F)ccc3C2=O)c(C)n1] | MS (m/z) 428.0 (M + H)⁺ | 1-bromo-2- methylbenz- ene | 3-(6-methoxy- 2- methylpyridin- 3-yl)-7- (trifluoromethyl)- 2,3- dihydroquinazo- lin-4(1H)-one |
| 815 | 1-(5-fluoro-2- methylphenyl)- 3-(6-methoxy- 2- methylpyridin- 3-yl)-6- (trifluoromethyl)- 2,3- dihydroquinazo- lin-4(1H)-one | 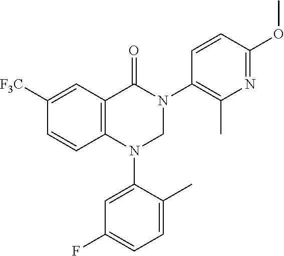 [SMILES: COc1ccc(N2CN(c3cc(F)ccc3C)c3ccc(C(F)(F)F)cc3C2=O)c(C)n1] | MS (m/z) 446.2 (M + H)⁺ | 2-bromo-4- fluoro-1- methylbenz- ene | 3-(6-methoxy- 2- methylpyridin- 3-yl)-6- (trifluoromethyl)- 2,3- dihydroquinazo- lin-4(1H)-one |
| 816 | 1-(3-fluoro-2- methylphenyl)- 3-(6-methoxy- 2- methylpyridin- 3-yl)-6- (trifluoromethyl)- 2,3- dihydroquinazo- lin-4(1H)-one | 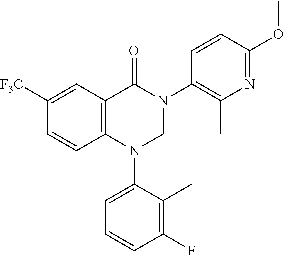 [SMILES: COc1ccc(N2CN(c3cccc(F)c3C)c3ccc(C(F)(F)F)cc3C2=O)c(C)n1] | MS (m/z) 446.0 (M + H)⁺ | 1-bromo-3- fluoro-2- methylbenz- ene | 3-(6-methoxy- 2- methylpyridin- 3-yl)-6- (trifluoromethyl)- 2,3- dihydroquinazo- lin-4(1H)-one |

### Intermediate 817

### 2-((4-Fluoro-2-methylphenyl)amino)-N-(4-methyl-6-oxo-1,6-dihydropyridazin-3-yl)-4-(trifluoromethyl)benzamide

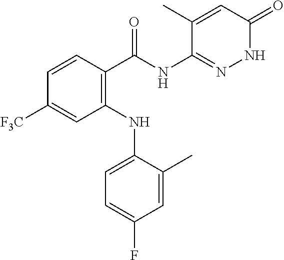 [SMILES: Cc1cc(F)ccc1Nc1cc(C(F)(F)F)ccc1C(=O)Nc1n[nH]c(=O)cc1C]

To a stirred solution of 2-((4-fluoro-2-methylphenyl)amino)-N-(6-methoxy-4-methylpyridazin-3-yl)-4-(trifluoromethyl)benzamide (1.00 g, 2.302 mmol) in DMF (50 mL) under nitrogen at 0° C., lithium chloride (0.488 g, 11.51 mmol) and p-toluenesulfonic acid monohydrate (2.189 g, 11.51 mmol) were added. The reaction mixture was stirred at 100° C. for 1.5 hours. Upon completion, the reaction mass was allowed to cool to room temperature and poured into water (300 mL). The mixture was stirred at room temperature for 30 mins. The precipitate was filtered and dried under vacuo to give the title compound as an off-white solid (0.94 g, 2.221 mmol, 96% yield). MS (m/z) 420.8 (M)⁺.

### Intermediate 818

### 1-(4-Fluoro-2-methylphenyl)-3-(6-methoxy-2-methylpyridin-3-yl)-7-(trifluoromethyl)-2,3-dihydroquinazoline-4(1H)-thione

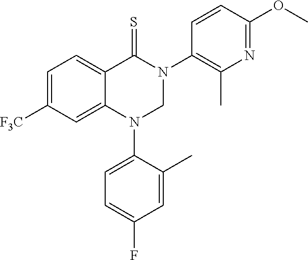 [SMILES: COc1ccc(N2CN(c3ccc(F)cc3C)c3cc(C(F)(F)F)ccc3C2=S)c(C)n1]

To a mixture of 1-(4-fluoro-2-methylphenyl)-3-(6-methoxy-2-methylpyridin-3-yl)-7-(trifluoromethyl)-2,3-dihydroquinazolin-4(1H)-one (200 mg, 0.449 mmol) in toluene (4 mL) was added Lawesson's reagent (109 mg, 0.269 mmol). The reaction was stirred at 80° C. for 2 hours. Solvent was removed in vacuo and the crude product was purified by column chromatography (Isco, 24 g silica column, EtOAc/heptane 0% to 40%) to give the title compound (158 mg, 0.312 mmol, 69.4% yield). LCMS: 462.3 (M+H)⁺

### Intermediate 819

### 3-(6-methoxy-2-methylpyridin-3-yl)-1-(2-methyl-4-(trifluoromethoxy)phenyl)-6-(trifluoromethyl)-2,3-dihydropyrido[2,3-d]pyrimidin-4(1H)-one

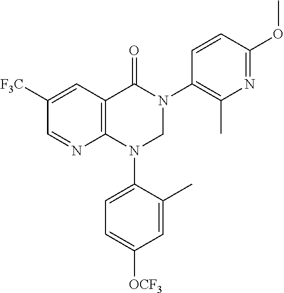 [SMILES: COc1ccc(N2CN(c3ccc(OC(F)(F)F)cc3C)c3ncc(C(F)(F)F)cc3C2=O)c(C)n1]

To a solution containing N-(6-methoxy-2-methylpyridin-3-yl)-2-((2-methyl-4-(trifluoromethoxy)phenyl)amino)-5-(trifluoromethyl)nicotinamide (43.09 g, 86 mmol) and Cs₂CO₃ (112 g, 344 mmol) in DMF (861 mL) at RT was added diiodomethane (20.84 mL, 258 mmol). Reaction was warmed to 100° C. for 18 hr. Additional diiodomethane (6.95 mL, 86 mmol) was added and the reaction was stirred for 18 hr further (total of 48 hr). The reaction was cooled and diluted with water and extracted with ethyl acetate. The combined organic phases were dried over MgSO₄, filtered and concentrated. The crude product was divided into 2 batches to purify. Purification by flash chromatography on SiO₂ (330 g) with 0→30% EtOAc in heptane using 10% step gradient (4 column volumes at each step) as eluant afforded the title compound as a tan foam (26.79 g, 52.3 mmol, 61% yield). MS (m/z) 513.2 (M+H)⁺.

### Intermediate 820

### 2-((4-fluoro-2-methylphenyl)amino)-5-(trifluoromethyl)nicotinic Acid

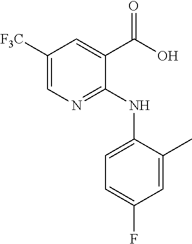 [SMILES: Cc1cc(F)ccc1Nc1ncc(C(F)(F)F)cc1C(=O)O]

To a solution containing 4-fluoro-2-methylaniline (2.66 g, 21.3 mmol) in Acetic Acid (40 mL) was added 2-chloro-5-(trifluoromethyl)nicotinic acid (3 g, 13.3 mmol). The reaction was warmed to 100° C. for 18 hr. The reaction was cooled to RT and treated with 1N KOH and solid KOH to pH=12. The resulting solids were filtered and filtrate was acidified with 1N HCl to pH=3. The resulting solid was filtered and washed with water. Solid was taken up in DCM, ethyl acetate and minimal THF, dried over Na₂SO₄, filtered and concentrated. Residue was taken up in hexanes and filtered and washed with hexanes to afford title compound as a yellow solid (2.86 g, 9.1 mmol, 68% yield). MS (m/z) 315.1 (M+H)⁺.

### Intermediate 821

### 2-((4-fluoro-2-methylphenyl)amino)-N-(6-methoxy-2-methylpyridin-3-yl)-5-(trifluoromethyl)nicotinamide

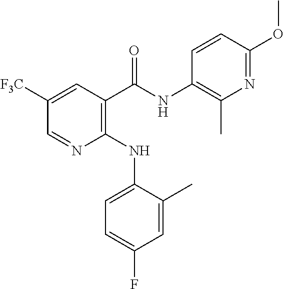 [SMILES: COc1ccc(NC(=O)c2cc(C(F)(F)F)cnc2Nc2ccc(F)cc2C)c(C)n1]

To a solution containing 6-methoxy-2-methylpyridin-3-amine (0.66 g, 4.8 mmol) in acetonitrile (13.3 mL) was added 2-((4-fluoro-2-methylphenyl)amino)-5-(trifluoromethyl)nicotinic acid (1.25 g, 4.0 mmol) followed by HATU (2.27 g, 6.0 mmol) and DIPEA (3.47 mL, 19.9 mmol). The reaction was stirred at RT for 1 hr. Reaction was diluted with NH₄Cl aq. and extracted with ethyl acetate, dried over MgSO₄, filtered and concentrated. Purification by flash chromatography on SiO₂ (40 g) with 0→30% ethyl acetate in heptanes as eluant afforded title compound as a yellow solid (1.17 g, 2.7 mmol, 68% yield). MS (m/z) 435.1 (M+H)⁺.

### Intermediate 822

### 1-(4-fluoro-2-methylphenyl)-3-(6-methoxy-2-methylpyridin-3-yl)-6-(trifluoromethyl)-2,3-dihydropyrido[2,3-d]pyrimidin-4(1H)-one

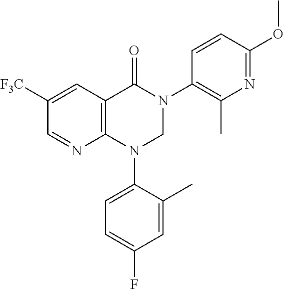 [SMILES: COc1ccc(N2CN(c3ccc(F)cc3C)c3ncc(C(F)(F)F)cc3C2=O)c(C)n1]

To a solution containing 2-((4-fluoro-2-methylphenyl)amino)-N-(6-methoxy-2-methylpyridin-3-yl)-5-(trifluoromethyl)nicotinamide (1.17 g, 2.7 mmol) and Cs₂CO₃ (3.51 g, 10.8 mmol) in acetonitrile (26.9 mL) at RT was added diiodomethane (0.65 mL, 8.1 mmol). Reaction was warmed to 80° C. for 18 hr. Reaction mixture was filtered through celite, washed with ethyl acetate and concentrated. Purification by flash chromatography on SiO₂ (120 g) with 0→30% ethyl acetate in heptane as eluant afforded title compound as a colorless foam (370 mg, 0.83 mmol, 31% yield). MS (m/z) 447.2 (M+H)⁺.

### Intermediate 823

### 1-(4-fluoro-2-methylphenyl)-3-(6-methoxy-2-methylpyridin-3-yl)-6-(trifluoromethyl)-2,3-dihydroquinazolin-4(1H)-one

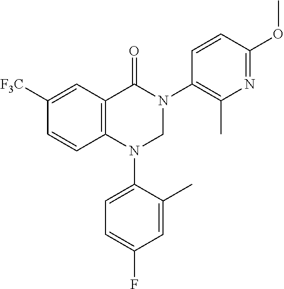 [SMILES: COc1ccc(N2CN(c3ccc(F)cc3C)c3ccc(C(F)(F)F)cc3C2=O)c(C)n1]

To a solution of 2-((4-fluoro-2-methylphenyl)amino)-N-(6-methoxy-2-methylpyridin-3-yl)-5-(trifluoromethyl)benzamide (2.4 g, 5.5 mmol) and Cs₂CO₃ (7.22 g, 22.2 mmol) in acetonitrile (25 mL) under nitrogen at RT was added diiodomethane (1.34 mL, 16.61 mmol) dropwise over 5 min. The reaction mixture was stirred at 80° C. for 16 h. The reaction mixture was cooled to RT and filtered through celite pad. The filtrate was concentrated onto SiO₂. Purification by flash chromatography on SiO₂ (50 g) with 0→100% EtOAc/petroleum ether as eluant afforded the title compound as a colorless solid (2.0 g, 4.1 mmol, 74% yield). MS (m/z) 446.0 (M+H)⁺.

### Intermediate 824

### N-(6-methoxy-2-methylpyridin-3-yl)-2-((2-methyl-4-(trifluoromethoxy)phenyl)amino)-5-(trifluoromethyl)nicotinamide

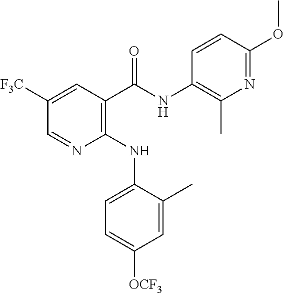 [SMILES: COc1ccc(NC(=O)c2cc(C(F)(F)F)cnc2Nc2ccc(OC(F)(F)F)cc2C)c(C)n1]

To a solution containing 6-methoxy-2-methylpyridin-3-amine (35.2 g, 255 mmol) in acetonitrile (654 mL) was added 2-((2-methyl-4-(trifluoromethoxy)phenyl)amino)-5-(trifluoromethyl)nicotinic acid (74.6 g, 196 mmol) followed by HATU (112 g, 294 mmol) and DIPEA (171 mL, 981 mmol). The reaction was stirred at RT for 10 min. Reaction mixture was diluted with water (1000 mL), extracted with ethyl acetate and the organic phase was washed with brine, dried over MgSO₄, filtered and concentrated. The concentrated solution was taken up in minimal DCM and diluted with heptanes—a light yellow solid and a dark brown solid precipitated. The light yellow solid was carefully decanted and washed with heptanes to afford product. The remaining dark solid was taken up in DCM and purification by flash chromatography on SiO₂ (330 g) with 0→10% ethyl acetate in dichloromethane as eluant afforded product. Filtrate from above was also concentrated and purification by flash chromatography in batches on SiO₂ (330 g) with 0→10% ethyl acetate in dichloromethane as eluant afforded product. All batches of product were combined and dried to afford title compound as a pale yellow solid (93 g, 186 mmol, 95% yield). MS (m/z) 501.2 (M+H)⁺.

### Intermediate 825

### 1-(4-Fluoro-2-methylphenyl)-3-(6-methoxy-2-methylpyridin-3-yl)-7-(oxetan-3-yl)-2,3-dihydroquinazolin-4(1H)-one

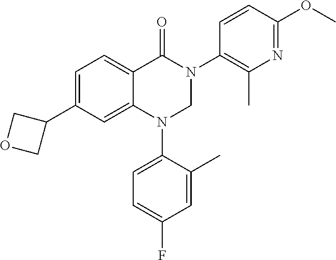 [SMILES: COc1ccc(N2CN(c3ccc(F)cc3C)c3cc(C4COC4)ccc3C2=O)c(C)n1]

### Step 1: 1-(4-Fluoro-2-methylphenyl)-3-(6-methoxy-2-methylpyridin-3-yl)-7-(4,4,5,5-tetramethyl-1,3,2-dioxaborolan-2-yl)-2,3-dihydroquinazolin-4(1H)-one

A suspension of 7-bromo-1-(4-fluoro-2-methylphenyl)-3-(6-methoxy-2-methylpyridin-3-yl)-2,3-dihydroquinazolin-4(1H)-one (0.5 g, 1.096 mmol), bis(pinacolato)diboron (0.362 g, 1.424 mmol), potassium acetate (0.213 g, 2.170 mmol) and PdCl₂(dppf-CH₂Cl₂ adduct (0.089 g, 0.110 mmol) in 1,4-dioxane (7.30 ml) was stirred at 80° C. overnight. The reaction was cooled and filtered through a pad of Celite. The filtrate was partitioned between water and EtOAc and the organic layer was dried over Na₂SO₄ and concentrated. The residue was purified by column chromatography (Isco, 24 g column, 0-23% EtOAc/heptane over 20 minutes) to give the title compound as an off-white solid (620 mg, 0.862 mmol, 79% yield). MS (m/z) 504.4 (M+H)⁺.

### Step 2: 1-(4-Fluoro-2-methylphenyl)-3-(6-methoxy-2-methylpyridin-3-yl)-7-(oxetan-3-yl)-2,3-dihydroquinazolin-4(1H)-one

To a microwave vial were added 1-(4-fluoro-2-methylphenyl)-3-(6-methoxy-2-methylpyridin-3-yl)-7-(4,4,5,5-tetramethyl-1,3,2-dioxaborolan-2-yl)-2,3-dihydroquinazolin-4(1H)-one (0.3 g, 0.596 mmol), sodium bis(trimethylsilyl)amide (0.131 g, 0.715 mmol), nickel(II) iodide (0.019 g, 0.060 mmol), (1R,2R)-2-aminocyclohexan-1-ol, hydrochloride (9.04 mg, 0.060 mmol) and degassed isopropanol (3 ml). The reaction mixture was degassed for 5 minutes before 3-iodooxetane (0.052 ml, 0.596 mmol) was added then heated in a microwave at 120° C. for 1 hr. The reaction was cooled, filtered and concentrated. The residue was purified by column chromatography (Isco, 24 g column, 0-100% EtOAc/heptane) to give the title compound as a pale yellow solid (29 mg, 0.067 mmol, 11.23% yield). MS (m/z) 434.3 (M+H)⁺.

### Compound Examples

Examples 1 to 10 were prepared from the indicated benzamides by methods analogous to those described for Intermediate 591

| Ex. | Name | Structure | Characterization | Benzamide |
|---|---|---|---|---|
| 1 | 1-(4-fluoro-2- methylphenyl)-3- (1-methyl-6-oxo- 1,6- dihydropyridin-3- yl)-7- (trifluoromethyl)- 2,3-dihydro- quinazolin- 4(1H)-one | 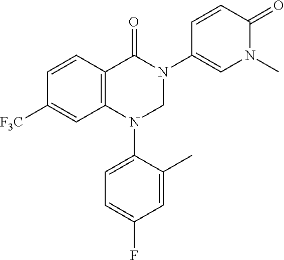 [SMILES: Cc1cc(F)ccc1N1CN(c2ccc(=O)n(C)c2)C(=O)c2ccc(C(F)(F)F)cc21] | ¹H NMR (400 MHz, DMSO-d₆) δ: 8.08 (d, J = 8.0 Hz, 1H), 7.89 (d, J = 2.9 Hz, 1H), 7.51 (dd, J = 9.6, 2.9 Hz, 1H), 7.41 (dd, J = 8.7, 5.4 Hz, 1H), 7.34 (dd, J = 9.8, 3.1 Hz, 1H), 7.28-7.18 (m, 2H), 6.46-6.36 (m, 2H), 5.39 (br. s., 1H), 5.03 (br. s., 1H), 3.41 (s, 3H), 2.23 (s, 3H). MS (m/z) 432.0 (M + H)⁺ | 2-((4-fluoro-2- methylphenyl)a- mino)-N-(1- methyl-6-oxo- 1,6- dihydropyridin-3- yl)-4- (trifluoromethyl)benz- amide |
| 2 | 1-(4-fluoro-2- methylphenyl)-3- (pyridin-3-yl)-7- (trifluoromethyl)- 2,3- dihydroquinazolin- 4(1H)-one | 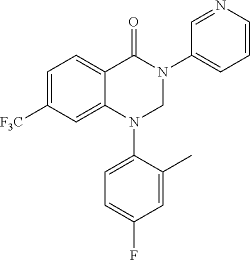 [SMILES: Cc1cc(F)ccc1N1CN(c2cccnc2)C(=O)c2ccc(C(F)(F)F)cc21] | ¹H NMR (400 MHz, DMSO-d₆) δ: 8.67 (d, J = 2.3 Hz, 1H), 8.47 (dd, J = 4.7, 1.4 Hz, 1H), 8.13 (d, J = 8.1 Hz, 1H), 7.85 (ddd, J = 8.1, 2.5, 1.5 Hz, 1H), 7.51-7.46 (m, 1H), 7.43 (dd, J = 8.9, 5.6 Hz, 1H), 7.33 (dd, J = 9.9, 3.0 Hz, 1H), 7.29 (dd, J = 8.1, 1.3 Hz, 1H), 7.20 (td, J = 8.4, 3.0 Hz, 1H), 6.46- 6.42 (m, 1H), 5.56 (br s, 1H), 5.29 (br s, 1H), 2.24 (s, 3H). MS (m/z) 402.3 (M + H)⁺ | 2-((4-fluoro-2- methylphenyl)a- mino)-N-(pyridin- 3-yl)-4- (trifluoromethyl)benz- amide |
| 3 | 1-(4-fluoro-2- methylphenyl)-3- (2- methoxypyrimidin- 5-yl)-7- (trifluoromethyl)- 2,3- dihydroquinazolin- 4(1H)-one | 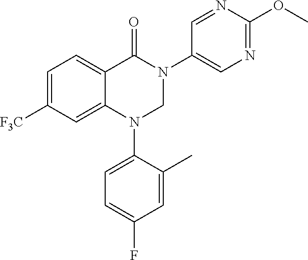 [SMILES: COc1ncc(N2CN(c3ccc(F)cc3C)c3cc(C(F)(F)F)ccc3C2=O)cn1] | ¹H NMR (400 MHz, DMSO-d₆) δ: 8.69 (s, 2H), 8.11 (d, J = 7.83 Hz, 1 H), 7.43 (dd, J = 8.68, 5.50 Hz, 1 H), 7.33 (dd, J = 9.54, 2.93 Hz, 1 H), 7.27 (dd, J = 8.19, 1.10 Hz, 1 H), 7.21 (td, J = 8.50, 3.06 Hz, 1 H), 5.54 (br s, 1 H) 6.42 (s, 1 H), 5.27 (br s, 1 H), 3.94 (s, 3 H), 2.24 (s, 3 H). MS (m/z) 433.3 (M + H)⁺ | 2-((4-fluoro-2- methylphenyl)a- mino)-N-(2- methoxypyrimidin- 5-yl)-4- (trifluoromethyl)benz- amide |
| 4 | 1-(4-fluoro-2- methylphenyl)-3- (1-methyl-1H- pyrazol-5-yl)-6- (trifluoromethyl)- 2,3- dihydroquinazolin- 4(1H)-one | 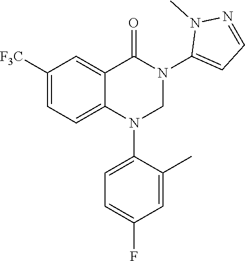 [SMILES: Cc1cc(F)ccc1N1CN(c2ccnn2C)C(=O)c2cc(C(F)(F)F)ccc21] | ¹H NMR (CHLOROFORM-d) δ: 8.40 (br s, 1H), 7.48-7.59 (m, 2H), 7.19 (br dd, J = 7.1, 4.2 Hz, 1H), 7.09- 7.15 (m, 1H), 7.00-7.08 (m, 1H), 6.38-6.46 (m, 1H), 6.19 (br d, J = 2.0 Hz, 1H), 5.27-5.37 (m, 1H), 4.86-4.97 (m, 1H), 3.81 (br d, J = 3.9 Hz, 3H), 2.31 (br d, J = 3.4 Hz, 3H). MS (m/z) 405.4 (M + H)⁺ | 2-((4-fluroo-2- methylphenyl)a- mino)-N-(1- methyl-1H- pyrazol-5-yl)-5- (trifluoromethyl)benz- amide |
| 5 | N-(3-(1-(4-fluoro- 2-methylphenyl)- 4-oxo-7- (trifluoromethyl)- 1,4- dihydroquinazolin- 3(2H)- yl)phenyl)aceta- mide | 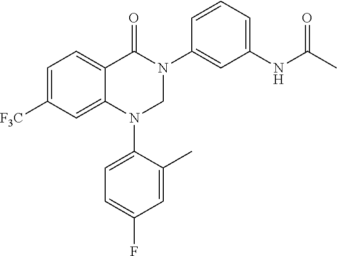 [SMILES: CC(=O)Nc1cccc(N2CN(c3ccc(F)cc3C)c3cc(C(F)(F)F)ccc3C2=O)c1] | ¹H NMR (DMSO-d₆, 400 MHz) δ: 10.05 (s, 1H), 8.12 (d, J = 8.0 Hz, 1H), 7.70-7.64 (m, 1H), 7.51-7.46 (m, 1H), 7.43 (dd, J = 8.7, 5.4 Hz, 1H), 7.37-7.30 (m, 2H), 7.27 (dd, J = 8.2, 1.1 Hz, 1H), 7.20 (td, J = 8.4, 2.9 Hz, 1H), 7.05 (dd, J = 8.1, 1.1 Hz, 1H), 6.42 (s, 1H), 5.51 (br s, 1H), 5.10 (br s, 1H), 2.23 (s, 3H), 2.04 (s, 3H) MS (m/z) 457.9 (M)⁺ | N-(3- acetamidophenyl)- 2-((4-fluoro-2- methylphenyla- mino)-4- (trifluoromethyl)benz- amide |
| 6 | N-(4-(1-(4-fluoro- 2-methylphenyl)- 4-oxo-7- (trifluoromethyl)- 1,4- dihydroquinazolin- 3(2H)- yl)phenyl)aceta- mide | 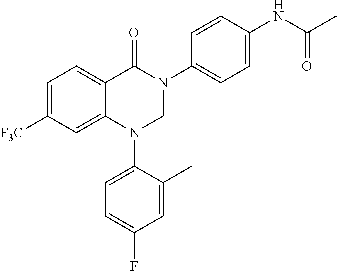 [SMILES: CC(=O)Nc1ccc(N2CN(c3ccc(F)cc3C)c3cc(C(F)(F)F)ccc3C2=O)cc1] | ¹H NMR (DMSO-d₆, 400 MHz) δ: 10.04 (s, 1H), 8.11 (d, J = 8.0 Hz, 1H), 7.60 (d, J = 8.9 Hz, 2H), 7.42 (dd, J = 8.8, 5.5 Hz, 1H), 7.36-7.29 (m, 3H), 7.26 (dd, J = 8.1, 1.1 Hz, 1H), 7.20 (td, J = 8.5, 2.8 Hz, 1H), 6.41 (s, 1H), 5.48 (br s, 1H), 5.10 (br s, 1H), 2.23 (s, 3H), 2.04 (s, 3H) MS (m/z) 457.9 (M)⁺ | N-(4- acetamidophenyl)- 2-((4-fluoro-2- methylphenyl)a- mino)-4- (trifloromethyl)benz- amide |
| 7 | 5-(6-chloro-5- fluoro-1-(4- fluoro-2- methylphenyl)-4- oxo-1,4- dihydroquinazolin- 3(2H)- yl)picolinamide | 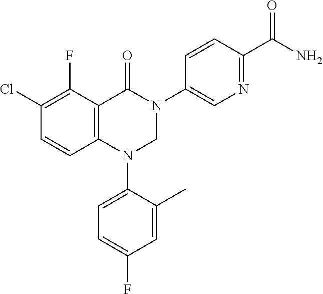 [SMILES: Cc1cc(F)ccc1N1CN(c2ccc(C(N)=O)nc2)C(=O)c2c1ccc(Cl)c2F] | ¹H NMR (400 MHz, DMSO-d6) δ: 8.68 (s, 1H), 8.12-8.07 (m, 2H), 7.94 (d, J = 8.00 Hz, 1H), 7.64 (s, 1H), 7.37 (dd, J = 8.60, 5.20 Hz, 1H), 7.38-7.35 (m, 1H), 7.30- 7.27 (m, 1H), 7.19-7.14 (m, 1H), 6.17 (dd, J = 0.80, 9.20 Hz, 1H), 5.47- 5.33 (m, 2H), 2.21 (s, 3H). MS (m/z) 429.0 (M + H)⁺ | 5-(3-chloro-2- fluoro-6-((4- fluoro-2- methylphenyl)a- mino)benzamido) picolinamide |
| 8 | 4-(6-chloro-5- fluoro-1-(4- fluoro-2- methylphenyl)-4- oxo-1,4- dihydroquinazolin- 3(2H)- yl)picolinamide | 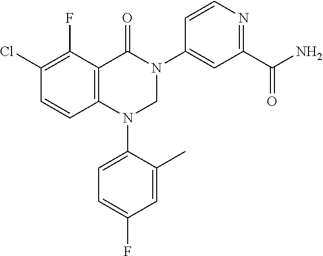 [SMILES: Cc1cc(F)ccc1N1CN(c2ccnc(C(N)=O)c2)C(=O)c2c1ccc(Cl)c2F] | ¹H NMR (400 MHz, DMSO-d6): δ 8.60 (d, J = 0.40 Hz, 1H), 8.14 (d, J = 2.40 Hz, 1H), 8.07 (d, J = 2.00 Hz, 1H), 7.70 (d, J = 2.40 Hz, 1H), 7.63 (dd, J = 5.60, 2.40 Hz, 1H), 7.55 (dd, J = 9.20, 7.60 Hz, 1H), 7.38 (dd, J = 8.60, 5.60 Hz, 1H), 7.29 (dd, J = 9.60, 2.80 Hz, 1H), 7.20-7.15 (m, 1H), 6.16 (dd, J = 9.00, 0.80 Hz, 1H), 5.58-5.28 (m, 2H), 2.19 (s, 3H). MS (m/z) 429.0 (M + H)⁺ | 4-(3-chloro-2- fluoro-6-((4- fluoro-2- methylphenyl)a- mino)benzamido) picolinamide |
| 9 | 1-(2-bromo-4- fluorophenyl)-3- (6-methoxy-2- methylpyridin-3- yl)-6- (trifluoromethyl)- 2,3- dihydroquinazolin- 4(1H)-one | 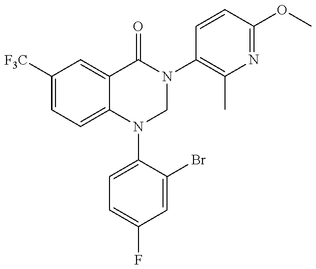 [SMILES: COc1ccc(N2CN(c3ccc(F)cc3Br)c3ccc(C(F)(F)F)cc3C2=O)c(C)n1] | ¹H NMR (400 MHz, DMSO-d6): δ: 8.12 (s, 1H), 7.87 (dd, J = 2.80, 8.20 Hz, 1H), 7.71-7.64 (m, 3H), 7.51-7.42 (m, 1H), 6.74 (d, J = 8.80 Hz, 1H), 6.50-6.40 (m, 1H), 5.67-4.86 (m, 2H), 3.85 (s, 3H), 2.36-2.33 (m, 3H). MS (m/z) 510.0 (M + H)⁺ | 2-((2-bromo-4- fluorophenyl)ami- no)-N-(6- methoxy-2- methylpyridin-3- yl)-5- (trifluoromethyl)benz- amide |
| 10 | 1-(4-fluoro-2- methylphenyl)-3- (6- methoxypyridin- 3-yl)-7- (trifluoromethyl)- 2,3- dihydroquinazolin- 4(1H)-one | 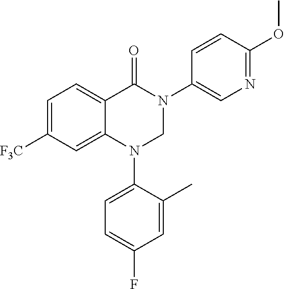 [SMILES: COc1ccc(N2CN(c3ccc(F)cc3C)c3cc(C(F)(F)F)ccc3C2=O)cn1] | ¹H NMR (400 MHz, DMSO-d₆) δ: 8.21 (d, J = 2.4 Hz, 1H), 8.11 (d, J = 8.0 Hz, 1H), 7.76 (dd, J = 8.8, 2.8 Hz, 1H), 7.42 (dd, J = 8.7, 5.4 Hz, 1H), 7.32 (dd, J = 9.6, 2.9 Hz, 1H), 7.26 (dd, J = 8.1, 1.0 Hz, 1H), 7.20 (td, J = 8.5, 2.9 Hz, 1H), 6.89 (d, J = 8.9 Hz, 1H), 6.42 (s, 1H), 5.48 (br s, 1H), 5.16 (br s, 1H), 3.86 (s, 3H), 2.24 (s, 3H). MS (m/z) 432.0 (M + H)⁺ | 2-((4-fluoro-2- methylphenyl)a- mino)-N-(6- methoxypyridin- 3-yl)-4- (trifluoromethyl)benz- amidee |

Examples 11-22 were prepared from the indicated benzamide by methods analogous to those described for Intermediate 756

| Ex. | Name | Structure | Characterization | Benzamide |
|---|---|---|---|---|
| 11 | 6-chloro-7- fluoro-1-(2- methyl-4- (trifluoromethoxy) phenyl)-3-(3- methylpyridin-4- yl)-2,3- dihydroquinazolin- 4(1H)-one | 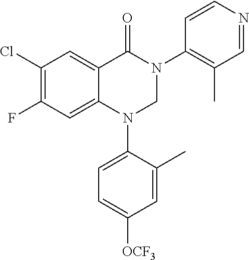 [SMILES: Cc1cnccc1N1CN(c2ccc(OC(F)(F)F)cc2C)c2cc(F)c(Cl)cc2C1=O] | ¹H NMR (400 MHz, DMSO-d₆) δ: 8.52 (s, 1H), 8.46 (d, J = 4.9 Hz, 1H), 8.00 (d, J = 8.3 Hz, 1H), 7.49-7.40 (m, 2H), 7.36-7.29 (m, 2H), 6.31 (br d, J = 10.8 Hz, 1H), 5.31 (d, J = 1.0 Hz, 2H), 2.27 (s, 3H), 2.16 (s, 3H). MS (m/z) 466.3 (M + H)⁺ | 5-chloro-4- fluoro-2-((2- methyl-4- (trifluoromethoxy) phenyl)amino)- N-(3- methylpyridin-4- yl)benzamide |
| 12 | 6-chloro-1-(2- ethyl-4- fluorophenyl)-7- fluoro-3-(3- methylpyridin-4- yl)-2,3- dihydroquinazolin- 4(1H)-one | 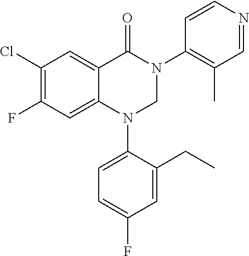 [SMILES: CCc1cc(F)ccc1N1CN(c2ccncc2C)C(=O)c2cc(Cl)c(F)cc21] | ¹H NMR (400 MHz, DMSO-d₆) δ: 8.53 (s, 1H), 8.47 (d, J = 5.4 Hz, 1H), 7.98 (d, J = 8.3 Hz, 1H), 7.42-7.39 (m, 1H), 7.32-7.29 (m, 2H), 7.20 (dt, J = 2.9, 8.3 Hz, 1H), 6.17 (d, J = 10.8 Hz, 1H), 5.56 (br s, 1H), 4.97 (br s, 1H), 2.58 (t, J = 7.7 Hz, 2H), 2.17 (s, 3H), 1.12 (t, J = 7.6 Hz, 3H). MS (m/z) 414.3 (M + H)⁺ | 5-chloro-2-((2- ethyl-4- fluorophenyl)ami- no)-4-fluoro-N- (3-methylpyridin- 4-yl)benzamide |
| 13 | 1-(4-fluoro-2- methylphenyl)-3- (4- (methylsulfonyl)phen- yl)-7- (trifluoromethyl)- 2,3- dihydroquinazolin- 4(1H)-one | 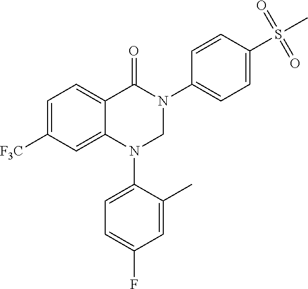 [SMILES: Cc1cc(F)ccc1N1CN(c2ccc(S(C)(=O)=O)cc2)C(=O)c2ccc(C(F)(F)F)cc21] | ¹H NMR (400 MHz, DMSO-d₆) δ: 8.16 (d, J = 8.1 Hz, 1H), 7.80 (dt, J = 8.9, 2.0 Hz, 2H), 7.70 (dt, J = 8.9, 2.0 Hz, 2H), 7.44 (dd, J = 8.7, 5.4 Hz, 1H), 7.3-7.4 (m, 2H), 7.21 (td, J = 8.9, 3.3 Hz, 1H), 6.4-6.5 (m, 1H), 5.59 (br s, 1H), 5.33 (br s, 1H), 3.25 (s, 3H), 2.23 (s, 3H) MS (m/z) 479.2 (M + H)⁺ | 2-((4-fluoro-2- methylphenyl)a- mino)-N-(4- (methylsulfonyl)phen- yl)-4- (trifluoromethyl)benz- amide |
| 14 | 1-(4-fluoro-2- methylphenyl)-3- (3- (methylsulfonyl)phen- yl)-7- (trifluoromethyl)- 2,3- dihydroquinazolin- 4(1H)-one | 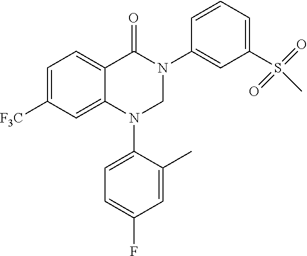 [SMILES: Cc1cc(F)ccc1N1CN(c2cccc(S(C)(=O)=O)c2)C(=O)c2ccc(C(F)(F)F)cc21] | ¹H NMR (400 MHz, DMSO-d₆) δ: 8.15 (d, J = 8.1 Hz, 1H), 7.96 (t, J = 1.8 Hz, 1H), 7.86-7.82 (m, 1H), 7.81-7.77 (m, 1H), 7.74-7.69 (m, 1H), 7.46 (dd, J = 8.7, 5.4 Hz, 1H), 7.34 (dd, J = 9.9, 3.0 Hz, 1H), 7.31-7.21 (m, 1H), 7.22 (td, J = 8.6, 3.0 Hz, 1H), 6.44-6.42 (m, 1H), 5.61 (br s, 1H),5.34 (br s, 1H), 3.26 (s, 3H), 2.24 (s, 3H) MS (m/z) 479.3 (M + H)⁺ | 2-((4-fluoro-2- methylphenyl)a- mino)-N-(3- (methylsulfonyl)phen- yl)-4- (trifluoromethyl)benz- amide |
| 15 | 3-(1,1- dioxidotetrahydro- thiophen-3-yl)- 1-(4-fluoro-2- methylphenyl)-6- (trifluoromethyl)- 2,3- dihydroquinazolin- 4(1H)-one | 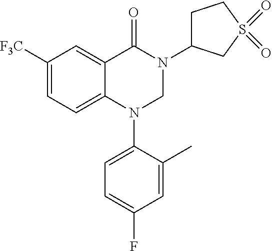 [SMILES: Cc1cc(F)ccc1N1CN(C2CCS(=O)(=O)C2)C(=O)c2cc(C(F)(F)F)ccc21] | ¹H NMR (400 MHz, CHLOROFORM-d) δ: 8.32 (d, J = 1.96 Hz, 1H), 7.50 (dd, J = 8.80, 1.96 Hz, 1H), 7.10-7.20 (m, 2H), 7.01-7.08 (m, 1H), 6.34 (dd, J = 8.56, 2.69 Hz, 1H), 5.44 (quin, J = 8.93 Hz, 1H), 4.95- 5.07 (m, 1H), 4.69-4.80 (m, 1H), 3.41-3.52 (m, 1H), 3.29-3.40 (m, 1H), 3.00-3.19 (m, 2H), 2.56 (ddd, J = 13.45, 7.34, 2.20 Hz, 1H), 2.26-2.42 (m, 1H), 2.25 (s, 3H). | N-(1,1- dioxidotetrahydro- thiophen-3-yl)- 2-((4-fluoro-2- methylphenyl)a- mino)-5- (trifluormethyl)benz- amide |
| | | | MS (m/z) 443.2 | |
| | | | (M + H)⁺ | |
| 16 | 1-(4-fluoro-2- methylphenyl)-3- (3-methylpyridin- 4-yl)-6- (trifluoromethyl)- 2,3- dihydroquinazolin- 4(1H)-one | 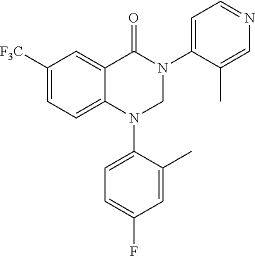 [SMILES: Cc1cnccc1N1CN(c2ccc(F)cc2C)c2ccc(C(F)(F)F)cc2C1=O] | ¹H NMR (400 MHz, CHLOROFORM-d) δ: 8.59 (s, 1H), 8.54 (d, J = 4.89 Hz, 1H), 8.42 (d, J = 2.45 Hz, 1H), 7.55 (dd, J = 8.56, 2.20 Hz, 1H), 7.17-7.22 (m, 1H), 7.16 (d, J = 4.89 Hz, 1H), 7.11 (dd, J = 9.05, 2.69 Hz, 1H), 7.04 (td, J = 8.31, 2.93 Hz, 1H), 6.41 (d, J = 8.80 Hz, 1H), 5.32- 5.44 (m, 1H), 4.87-4.98 (m, 1H), 2.31 (s, 3H), 2.30 (s, 3H). | 2-((4-fluoro-2- methylphenyl)a- mino)-N-(3- methylpyridin-4- yl)-5- (trifluoromethyl)benz- amide |
| | | | MS (m/z) 416.4 (M + H)⁺ | |
| 17 | 3-(1,1- dioxidotetrahydro- 2H-thiopyran- 4-yl)-1-(4-fluoro- 2-methylphenyl)- 7- (trifluoromethyl)- 2,3- dihydroquinazolin- 4(1H)-one | 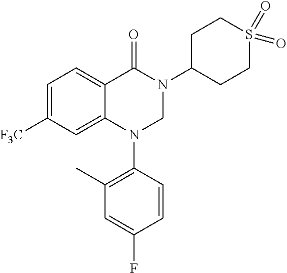 [SMILES: Cc1cc(F)ccc1N1CN(C2CCS(=O)(=O)CC2)C(=O)c2ccc(C(F)(F)F)cc21] | ¹H NMR (400 MHz, DMSO-d6) δ: 8.04 (d, J = 7.82 Hz, 1H), 7.38 (dd, J = 8.80, 5.38 Hz, 1H), 7.34 (dd, J = 9.78, 2.93 Hz, 1H), 7.16-7.25 (m, 2H), 6.31 (s, 1H), 5.05 (br d, J = 9.29 Hz, 1H), 4.73-4.82 (m, 1H), 4.70- 4.73 (m, 1H), 4.68 (br t, J = 3.18 Hz, 1H), 3.42 (br t, J = 13.45 Hz, 2H), 3.18 (d, J = 5.38 Hz, 1H), 3.10 (br s, 1H), 3.07 (br s, 1H), 2.28-2.40 (m, 1H), 2.19 (s, 2H), 1.98 (br d, | N-(1,1- dioxidotetrahydro- 2H-thiopyran- 4-yl)-2-((4-fluoro- 2- methylphenyl)a- mino)-4- (trifluoromethyl)benz- amide |
| | | | J = 11.25 Hz, 2H) | |
| | | | MS (m/z) 457.3 (M + H)⁺ | |
| 18 | 1-(4-fluoro-2- methylphenyl)-3- (1H-pyrazol-4- yl)-6- (trifluoromethyl)- 2,3- dihydroquinazolin- 4(1H)-one | 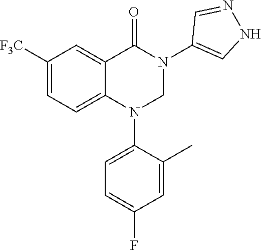 [SMILES: Cc1cc(F)ccc1N1CN(c2cn[nH]c2)C(=O)c2cc(C(F)(F)F)ccc21] | ¹H NMR (400 MHz, CHLOROFORM-d) δ: 8.38 (d, J = 1.5 Hz, 1H), 8.23 (s, 1H), 8.16 (br s, 1H), 7.70 (s, 1H), 7.48- 7.55 (m, 1H), 7.25 (br dd, J = 8.6, 5.1 Hz, 1H), 7.12- 7.18 (m, 1H), 7.08 (ddd, J = 11.4, 8.2, 2.9 Hz, 1H), 6.36 (dd, J = 8.3, 6.4 Hz, 1H), 5.34-5.42 (m, 1H), 5.10 (br s, 1H), 2.27 (s, 3H). MS (m/z) 391.3 (M + H)⁺ | tert-butyl 4-(2- ((4-fluoro-2- methylphenyl)a- mino)-5- (trifluoromethyl)benz- amido)-1H- pyrazole-1- carboxylate |
| 19 | 1-(4-fluoro-2- methylphenyl)-3- (6-methoxy-2- methylpyridin-3- yl)-6- (trifluoromethyl)- 2,3- dihydroquinazolin- 4(1H)-one | 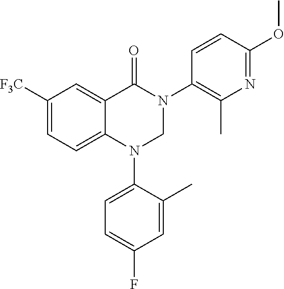 [SMILES: COc1ccc(N2CN(c3ccc(F)cc3C)c3ccc(C(F)(F)F)cc3C2=O)c(C)n1] | ¹H NMR (400 MHz, DMSO-d₆) δ ppm 8.10 (m, H), 7.67 (m, 1 H), 7.42 (m, 1 H), 7.33 (m, 1 H), 7.21 (br s, 1 H), 6.74 (d, 1 H), 6.26-6.47 (m, 1 H), 5.63 (m, 1 H), 5.17 (m, 1 H), 4.78-4.90 (m, 1 H), 3.85 (s, 3 H), 2.33 (s, 3 H), 2.25 (s, 3 H) MS (m/z) 446.3 (M + H)⁺ | 2-((4-fluoro-2- methylphenyl)a- mino)-N-(6- methoxy-2- methylpyridin-3- yl)-5- (trifluoromethyl)benz- amide |
| 20 | 1-(4-fluoro-2- methylphenyl)-3- (4-methyl-6-oxo- 1,6- dihydropyridazin- 3-yl)-7- (trifluoromethyl)- 2,3- dihydroquinazolin- 4(1H)-one | 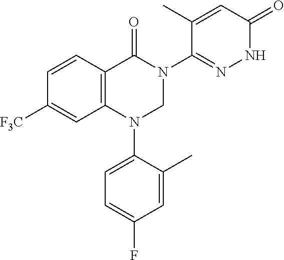 [SMILES: Cc1cc(F)ccc1N1CN(c2n[nH]c(=O)cc2C)C(=O)c2ccc(C(F)(F)F)cc21] | 1H-NMR (400 MHz, DMSO-d₆): δ 12.93 (s, 0.5H), 8.12-8.09 (m, 1H), 7.48 (br s, 1H), 7.35-7.32 (m, 1H), 7.27-7.15 (m, 2H), 6.84-6.92 (m, 1H), 6.76 (t, J = 7.60 Hz, 0.5 H), 6.60-6.30 (m, 1H), 5.67-5.03 (m, 2H), 2.23 (s, 3H), 2.08-2.06 (m, 3H). MS (m/z) 433.0 (M + H)⁺ | 2-((4-fluoro-2- methylphenyl)a- mino)-N-(4- methyl-6-oxo- 1,6- dihydropyridazin- 3-yl)-4- (trifluoromethyl)benz- amide |
| 21 | 1-(2-methyl-4- (trifluoromethoxy) phenyl)-3-(3- methylpyridin-4- yl)-6- (trifluoromethyl)- 2,3- dihydroquinazolin- 4(1H)-one | 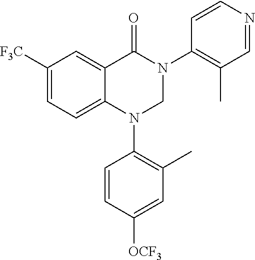 [SMILES: Cc1cnccc1N1CN(c2ccc(OC(F)(F)F)cc2C)c2ccc(C(F)(F)F)cc2C1=O] | ¹H-NMR (400 MHz, DMSO-d₆): δ 8.54 (s, 1H), 8.48 (d, J = 5.20 Hz, 1H), 8.14 (d, J = 2.00 Hz, 1H), 7.71 (dd, J = 2.40 Hz, 8.80 Hz, 1H), 7.51- 7.38 (m, 2H), 7.82-7.32 (m, 2H), 6.44 (d, J = 8.80 Hz, 1H), 5.59 (br s, 1H), 5.15 (br s, 1H), 2.26 (s, 3H), 2.18 (s, 3H). MS (m/z) 481.8 (M + H)⁺ | 2-((2-methyl-4- (trifluoromethoxy) phenyl)amino)- N-(3- methylpyridin-4- yl)-5- (trifluoromethyl)benz- amide |
| 22 | 3-(6-chloro-4- methylpyridin-3- yl)-1-(4-fluoro-2- methylphenyl)-6- (trifluoromethyl)- 2,3- dihydroquinazolin- 4(1H)-one | 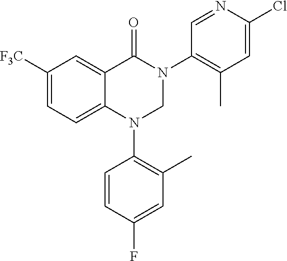 [SMILES: Cc1cc(Cl)ncc1N1CN(c2ccc(F)cc2C)c2ccc(C(F)(F)F)cc2C1=O] | ¹H-NMR (400 MHz, DMSO-d₆) δ: 8.38 (s, 1H), 8.11 (d, J = 2.00 Hz, 1H), 7.69 (dd, J = 2.40, 8.80 Hz, 1H), 7.58 (s, 1H), 7.40-7.43 (m, 1H), 7.33 (dd, J = 2.80, 9.60 Hz, 1H), 7.21 (s, 1H), 6.38 (s, 1H), 4.96-5.70 (m, 2H), 2.25 (s, 6H). MS (m/z) 449.6 (M)⁺ | N-(6-chloro-4- methylpyridin-3- yl)-2-((4-fluoro-2- methylphenyl)a- mino)-5- (trifluoroemthyl)benz- amide |

Examples 23-34 were prepared from the indicated amides by methods analogous to those described for Intermediate 780.

| Ex. | Name | Structure | Characterization | Amide |
|---|---|---|---|---|
| 23 | 6-chloro-7- (difluoromethyl)- 1-(4-fluoro-2- methylphenyl)-3- (3-methylpyridin- 4-yl)-2,3- dihydroquinazolin- 4(1H)-one | 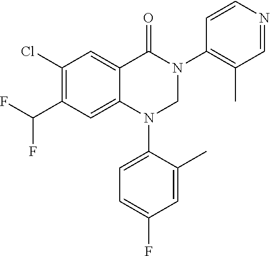 [SMILES: Cc1cnccc1N1CN(c2ccc(F)cc2C)c2cc(C(F)F)c(Cl)cc2C1=O] | ¹H NMR (400 MHz, DMSO-d₆) δ: 8.52-8.57 (m, 1 H) 8.48 (d, J = 4.89 Hz, 1 H) 7.97 (s, 1 H) 7.24-7.45 (m, 3 H) 7.19 (td, J = 8.44, 3.18 Hz, 1 H), 7.13 (t, J = 56 Hz, 1H), 6.51 (s, 1 H), 5.76 (s, 1 H), 5.04-5.65 (m, 2 H) 2.23 (s, 3 H) 2.17 (s, 3 H). MS (m/z) 432.3 (M + H)⁺ | 5-chloro-4- (difluoromethyl)- 2-((4-fluoro-2- methylphenyl)a- mino)-N-(3- methylpyridin-4- yl)benzamide |
| 24 | 1-(4-fluoro-2- methylphenyl)-3- (pyridin-3-yl)-6- (trifluoromethyl)- 2,3- dihydroquinazolin- 4(1H)-one | 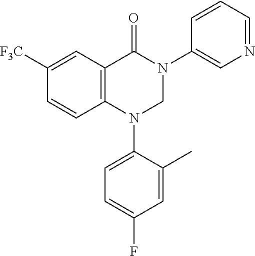 [SMILES: Cc1cc(F)ccc1N1CN(c2cccnc2)C(=O)c2cc(C(F)(F)F)ccc21] | ¹H NMR (400 MHz, DMSO-d6) δ: 8.63-8.71 (m, 1H) 8.47 (dd, J = 4.89, 1.47 Hz, 1H) 8.12-8.19 (m, 1H) 7.82-7.90 (m, 1 H) 7.65-7.73 (m, 1H) 7.41-7.51 (m, 2H) 7.29- 7.37 (m, 1H) 7.16-7.26 (m, 1H) 6.34-6.44 (m, 1 H) 5.51-5.70 (m, 1H) 5.27 (br d, J = 9.29 Hz, 1H) 2.18-2.31 (m, 3H). MS (m/z) 402.3 (M + H)⁺ | 2-((4-fluoro-2- methylphenyl)a- mino)-N-(pyridin- 3-yl)-5- (trifluoromethyl)benz- amide |
| 25 | 1-(4-fluoro-2- methylphenyl)-3- (4-methylpyridin- 3-yl)-6- (trifluoromethyl)- 2,3- dihydroquinazolin- 4(1H)-one | 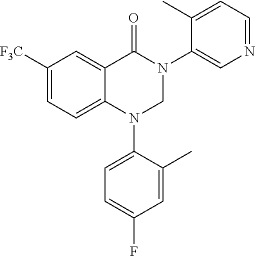 [SMILES: Cc1ccncc1N1CN(c2ccc(F)cc2C)c2ccc(C(F)(F)F)cc2C1=O] | ¹H NMR (400 MHz, DMSO-d₆) δ: 8.48-8.53 (m, 1H) 8.40-8.45 (m, 1H) 7.64-7.74 (m, 1H) 8.07-8.16 (m, 1H) 7.38- 7.47 (m, 2H) 7.30-7.36 (m, 1H) 7.15-7.25 (m, 1H) 6.39 (br s, 1H) 5.16- 5.39 (m, 1H) 4.80-5.03 (m, 1H) 2.20-2.31 (m, 6H) MS (m/z) 416.0 (M + H)⁺ | 2-((4-fluoro-2- methylphenyl)a- mino)-N-(4- methylpyridin-3- yl)-5- (trifluoromethyl)benz- amide |
| 26 | 1-(4-fluoro-2- methylphenyl)-3- (3- methylpyridazin- 4-yl)-6- (trifluoromethyl)- 2,3- dihydroquinazolin- 4(1H)-one | 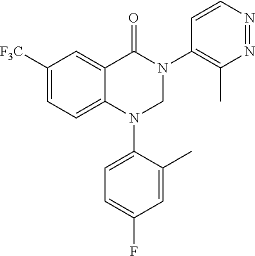 [SMILES: Cc1cc(F)ccc1N1CN(c2ccnnc2C)C(=O)c2cc(C(F)(F)F)ccc21] | ¹H NMR (400 MHz, DMSO-d₆) δ ppm 9.17- 9.22 (m, 1 H) 8.11-8.17 (m, 1 H) 7.65-7.75 (m, 2 H) 7.39-7.46 (m, 1 H) 7.30-7.36 (m, 1 H) 7.16- 7.26 (m, 1 H) 6.40- 6.47 (m, 1 H) 5.53-5.70 (m, 1 H) 5.15-5.29 (m, 1 H) 3.31-3.36 (m, 6 H) MS (m/z) 417.4 (M + H)⁺ | 2-((4-fluoro-2- methylphenyl)a- mino)-N-(3- methylpyridazin- 4-yl)-5- (trifluoromethyl)benz- amide |
| 27 | 7-chloro-6- fluoro-1-(4- fluoro-2- methylphenyl)-3- (6-methoxy-2- methylpyridin-3- yl)-2,3- dihydroquinazolin- 4(1H)-one | 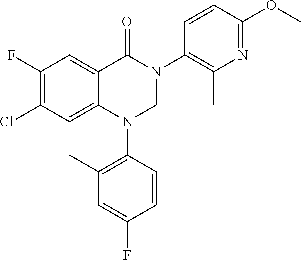 [SMILES: COc1ccc(N2CN(c3ccc(F)cc3C)c3cc(Cl)c(F)cc3C2=O)c(C)n1] | ¹H NMR (400 MHz, DMSO-d₆) δ: 7.79 (d, J = 9.29 Hz, 1H), 7.61 (d, J = 8.80 Hz, 1H), 7.24- 7.42 (m, 2H), 7.09-7.21 (m, 1H), 6.72 (d, J = 9.29 Hz, 1H), 6.24-6.50 (m, 1H), 5.49 (br s, 1H), 4.74- 4.89 (m, 1H), 3.84 (s, 3H), 2.28-2.32 (m, 3H), 2.26 (s, 3H). MS (m/z) 430.2 (M + H)⁺ | 4-chloro-5- fluoro-2-((4- fluoro-2- methylphenyl)a- mino)-N-(6- methoxy-2- methylpyridin-3- yl)benzamide |
| 28 | 1-(4-fluoro-2- methylphenyl)-3- (3-methyl-1H- pyrazol-4-yl)-6- (trifluoromethyl)- 2,3- dihydroquinazolin- 4(1H)-one | 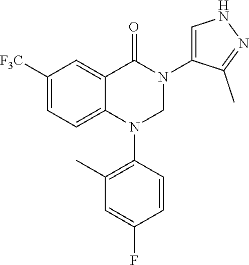 [SMILES: Cc1cc(F)ccc1N1CN(c2c[nH]nc2C)C(=O)c2cc(C(F)(F)F)ccc21] | ¹H NMR (400 MHz, DMSO-d₆) δ: 12.21- 13.16 (m, 1H), 8.10 (d, J = 1.96 Hz, 1H), 7.72- 7.87 (m, 1H), 7.65 (dd, J = 8.80, 1.96 Hz, 1H), 7.43 (dd, J = 8.56, 5.62 Hz, 1H), 7.33 (dd, J = 9.54, 2.69 Hz, 1H), 7.20 (td, J = 8.56, 2.93 Hz, 1H), 6.35 (d, J = 8.80 Hz, 1H), 5.42 (br d, J = 9.29 Hz, 1H), 4.89 (br d, J = 8.31 Hz, 1H), 3.34 (s, 3H), 2.23 (s, 3H) MS (m/z) 405.3 (M + H)⁺ | tert-butyl 4-(2- ((4-fluoro-2- mehtylphenyl)a- mino)-5- (trifluoromethyl)benz- amido)-3- methyl-1H- pyrazole-1- carboxylate |
| 29 | 3-(1- acetylpiperidin- 4-yl)-1-(4-fluoro- 2-methylphenyl)- 6- (trifluoromethyl)- 2,3- dihydroquinazolin- 4(1H)-one | 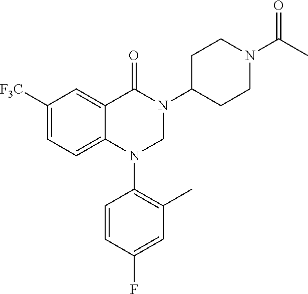 [SMILES: CC(=O)N1CCC(N2CN(c3ccc(F)cc3C)c3ccc(C(F)(F)F)cc3C2=O)CC1] | ¹H NMR (400 MHz, CHLOROFORM-d) δ: 8.33 (d, J = 2.45 Hz, 1H), 7.46 (dd, J = 8.56, 2.20 Hz, 1H), 7.09-7.22 (m, 2H), 6.96-7.08 (m, 1H), 6.19-6.41 (m, 1H), 4.82- 4.97 (m, 2H), 4.69- 4.80 (m, 1H), 4.54-4.64 (m, 1H), 3.83-4.01 (m, 1H), 3.06-3.34 (m, 1H), 2.58-2.78 (m, 1H), 2.13- 2.33 (m, 2H), 2.08- 2.13 (m, 3H), 1.77-1.97 (m, 2H), 1.55-1.68 (m, 2H), 1.38-1.53 (m, 1H). MS (m/z) 450.4 (M + H)⁺ | 3-(1- acetylpiperidin- 4-yl)-1-(4-fluoro- 2-methylphenyl)- 6- (trifluoromethyl)- 2,3- dihydroquinazolin- 4(1H)-one |
| 30 | 1-(4-fluoro-2- methylphenyl)-3- (pyridazin-4-yl)- 6- (trifluoromethyl)- 2,3- dihydroquinazolin- 4(1H)-one | 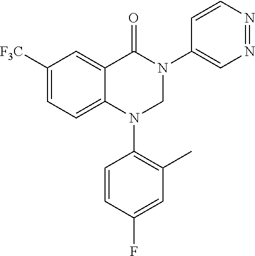 [SMILES: Cc1cc(F)ccc1N1CN(c2ccnnc2)C(=O)c2cc(C(F)(F)F)ccc21] | ¹H NMR (400 MHz, DMSO-d₆) δ: 9.48 (dd, J = 2.93, 0.98 Hz, 1H), 9.19 (dd, J = 5.87, 0.98 Hz, 1H), 8.18 (d, J = 1.96 Hz, 1 H), 7.76-7.67 (m, 2H), 7.46 (dd, J = 8.80, 5.38 Hz, 1H), 7.33 (dd, J = 9.29, 2.93 Hz, 1H), 7.22 (td, J = 8.44, 3.18 Hz, 1H), 6.40 (d, J = 8.80 Hz, 1H), 5.64 (br d, J = 10.27 Hz, 1H), 5.50-5.38 (m, 1H), 2.20 (s, 3H). MS (m/z) 403.3 (M + H)⁺ | 2-((4-fluoro-2- methylphenyl)a- mino)-N- (pyridazin-4-yl)- 5- (trifluoromethyl)benz- amide |
| 31 | 1-(4-fluoro-2- methylphenyl)-3- (5-methylpyridin- 3-yl)-6- (trifluoromethyl)- 2,3- dihydroquinazolin- 4(1H)-one | 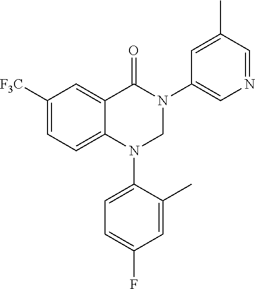 [SMILES: Cc1cncc(N2CN(c3ccc(F)cc3C)c3ccc(C(F)(F)F)cc3C2=O)c1] | ¹H NMR (400 MHz, CHLOROFORM-d) δ: 8.41 (t, J = 2.69 Hz, 2H), 8.37 (d, J = 0.98 Hz, 1H), 7.61 (t, J = 2.20 Hz, 1H), 7.48-7.57 (m, 1H), 7.22 (dd, J = 8.56, 5.14 Hz, 1H) 7.12, (dd, J = 8.80, 2.93 Hz, 1H), 7.00-7.07 (m, 1H), 6.40 (d, J = 8.80 Hz, 1H), 5.43 (br d, J = 9.29 Hz, 1H), 5.08 (br d, J = 9.29 Hz, 1H), 2.40 (s, 3H), 2.30 (s, 3H). MS (m/z) 416.4 (M + H)⁺ | 2-((4-fluoro-2- methylphenyl)a- mino)-N-(5- methylpyridin-3- yl)-5- (trifluoromethyl)benz- amide |
| 32 | 1-(4-fluoro-2- methylphenyl)-3- (2-methylpyridin- 3-yl)-6- (trifluoromethyl)- 2,3- dihydroquinazolin- 4(1H)-one | 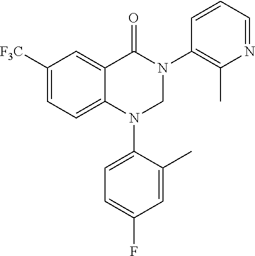 [SMILES: Cc1cc(F)ccc1N1CN(c2cccnc2C)C(=O)c2cc(C(F)(F)F)ccc21] | ¹H NMR (400 MHz, CHLOROFORM-d) δ: 8.54 (dd, J = 4.89, 1.47 Hz, 1H), 8.41 (d, J = 1.96 Hz, 1H), 7.59 (dd, J = 7.83, 1.47 Hz, 1H), 7.54 (dd, J = 8.80, 1.96 Hz, 1H), 7.26 (dd, J = 7.83, 4.89 Hz, 1H), 7.19 (br d, J = 3.91 Hz, 1H), 7.09-7.13 (m, 1H), 6.99-7.06 (m, 1H), 6.39 (br d, J = 5.87 Hz, 1H), 5.04-5.59 (m, 1H), 4.63- 4.83 (m, 1H), 2.58 (s, 3H), 2.32 (s, 3H). | 2-((4-fluoro-2- methylphenyl)a- mino)-N-(2- methylpyridin-3- yl)-5- (trifluoromethyl)benz- amide |
| | | | MS (m/z) 416.3 (M + H)⁺ | |
| 33 | 1-(4-fluoro-2- methylphenyl)-3- ((2R,3S)-2- methyl-6- oxopiperidin-3- yl)-6- (trifluoromethyl)- 2,3- dihydroquinazolin- 4(1H)-one | 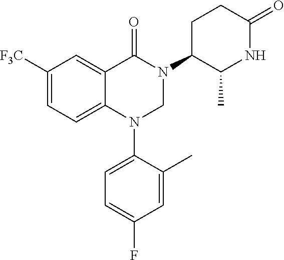 [SMILES: Cc1cc(F)ccc1N1CN([C@H]2CCC(=O)N[C@@H]2C)C(=O)c2cc(C(F)(F)F)ccc21] | ¹H NMR (600 MHz, METHANOL-d₄) δ 8.22 (s, 1H), 7.57 (dd, J = 8.8, 2.2 Hz, 1H), 7.3 (td, J = 8.5, 5.3 Hz, 1H), 7.21 (dd, J = 9.2, 2.9 Hz, 1H), 7.17-7.07 (m, 1H), 6.43- 6.35 (m, 1H), 5.18- 5.04 (m, 1H), 4.94-4.84 (m, 1H), 4.60-4.44 (m, 1H), 3.80-3.61 (m, 1H), 2.60-2.44 (m, 2H), 2.31- 2.24 (m, 3H), 2.15 (qd, J = 12.5, 5.9 Hz, 1H), 1.94 (dtd, J = 12.7, 6.5, 2.9 Hz, 1H), 1.43-1.18 | 2-((4-fluoro-2- methylphenyl)a- mino)-N- ((2R,3S)-2- methyl-6- oxopiperidin-3- yl)-5- (trifluoromethyl)benz- amide |
| | | | (m, 3H). | |
| | | | MS (m/z) 436.0 (M + H)⁺ | |
| 34 | 3-(1-(4-fluoro-2- methylphenyl)-4- oxo-6- (trifluoromethyl)- 1,4- dihydroquinazolin- 3(2H)- yl)piperidine-2,6- dione | 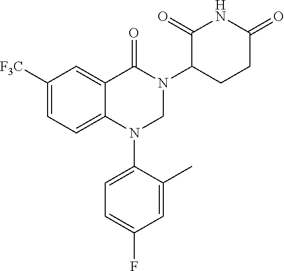 [SMILES: Cc1cc(F)ccc1N1CN(C2CCC(=O)NC2=O)C(=O)c2cc(C(F)(F)F)ccc21] | ¹H NMR (400 MHz, DMSO-d₆) δ: 10.92 (s, 1 H), 8.06 (br d, J = 7.50 Hz, 1 H), 7.62 (d, J = 8.91 Hz, 1 H), 7.43 (ddd, J = 17.01, 9.01, 5.50 Hz, 1 H), 7.32 (dd, J = 9.51, 3.00 Hz, 1 H), 7.20 (td, J = 8.50, 3.00 Hz, 1 H), 6.30 (t, J = 8.51 Hz, 1 H), 5.40-5.07 (m, 2H), 4.76 (dd, J = 32.52, 10.51 Hz, 1 H), 2.90-2.68 (m, 1 H), 2.53-2.57 (m, 1 H), 2.30- 2.48 (m, 1 H), 2.22 (d, J = 25.51 Hz, 3H), 1.97- 1.87 (m, 1 H). | N-(2,6- dioxopiperidin-3- yl)-2-((4-fluoro-2- methylphenyl)a- mino)-5- (trifluoromethyl)benz- amide |
| | | | MS (m/z) 436 (M + H)⁺ | |

### Example 35

### 1-(4-Fluoro-2-methylphenyl)-3-(2-methyl-6-oxo-1,6-dihydropyridin-3-yl)-7-(trifluoromethyl)-2,3-dihydroquinazolin-4(1H)-one

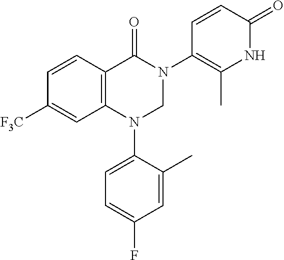 [SMILES: Cc1cc(F)ccc1N1CN(c2ccc(=O)[nH]c2C)C(=O)c2ccc(C(F)(F)F)cc21]

Iodotrimethylsilane (0.367 mL, 2.69 mmol) was added dropwise to a stirring solution of 1-(4-fluoro-2-methylphenyl)-3-(6-methoxy-2-methylpyridin-3-yl)-7-(trifluoromethyl)-2,3-dihydroquinazolin-4(1H)-one (300 mg, 0.674 mmol) in acetonitrile (25 mL) at 0° C. under N₂. The reaction mixture was stirred at 50° C. for 16 hours. The reaction was cooled to RT, diluted with EtOAc (20 mL) and stirred for 15 minutes. The precipitate was filtered and vacuum dried. The brown solid crude product was purified by column chromatography (Biotage, 20 g SNAP column, 0-15% MeOH/DCM over 45 minutes) to give the title compound as a yellow solid (92 mg, 0.213 mmol, 31.6% yield). ¹H NMR (400 MHz, DMSO-d₆) δ: 11.79 (br. s., 1H), 8.07 (d, J=8.0 Hz, 1H), 7.44-7.29 (m, 3H), 7.29-7.14 (m, 2H), 6.46-6.29 (m, 1H), 6.20 (d, J=9.5 Hz, 1H), 5.48 (br. s., 0.5H), 5.26-5.01 (m, 1H), 4.79 (br. s., 0.5H), 2.23 (s, 3H), 2.11 (br. s., 3H). MS (m/z) 432.0 (M+H)⁺.

Examples 36-143 were prepared from the indicated intermediate by methods analogous to those described for Example 35.

| Ex. | Name | Structure | Characterization | Intermediate |
|---|---|---|---|---|
| 36 | 1-(4-fluoro-2- methylphenyl)-3- (6-oxo-1,6- dihydropyridin-3- yl)-7- (trifluoromethyl)- 2,3- dihydroquinazolin- 4(1H)-one | 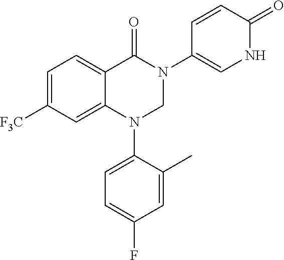 [SMILES: Cc1cc(F)ccc1N1CN(c2ccc(=O)[nH]c2)C(=O)c2ccc(C(F)(F)F)cc21] | ¹H NMR (400 MHz, DMSO-d₆) δ: 11.74 (br.s., 1H), 8.07 (d, J = 8.1 Hz, 1H), 7.53 (d, J = 2.6 Hz, 1H), 7.49 (dd, J = 9.6, 2.9 Hz, 1H), 7.39 (dd, J = 8.8, 5.5 Hz, 1H), 7.32 (dd, J = 9.6, 2.9 Hz, 1H), 7.27-7.15 (m, 2H), 6.40-6.32 (m, 2H), 5.37 (br.s., 1H), 5.06 (br.s., 1H), 2.23 (s, 3H). MS (m/z) 418.0 (M + H)⁺ | 1-(4-fluoro-2- methylphenyl)- 3-(6- methoxypyridin- 3-yl)-7- (trifluoromethyl)- 2,3- dihydroquinazolin- 4(1H)-one |
| 37 | 1-cyclohexyl-3-(6- oxo-1,6- dihydropyridin-3- yl)-7- (trifluoromethyl)- 2,3- dihydroquinazolin- 4(1H)-one | 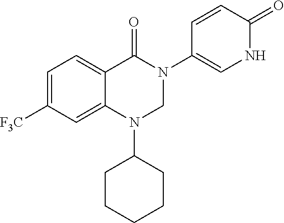 [SMILES: O=C1c2ccc(C(F)(F)F)cc2N(C2CCCCC2)CN1c1ccc(=O)[nH]c1] | ¹H NMR (400 MHz, DMSO-d₆) δ: 11.59 (br.s., 1H), 7.97 (d, J = 7.9 Hz, 1H), 7.50 (d, J = 2.5 Hz, 1H), 7.45 (dd, J = 9.6, 2.9 Hz, 1H), 7.37 (s, 1H), 7.15 (d, J = 8.1 Hz, 1H), 6.38 (d, J = 9.6 Hz, 1H), 4.85 (s, 2H), 3.78 (t, J = 11.3 Hz, 1H), 1.86- 1.69 (m, 4H), 1.66-1.37 (m, 5H), 1.19-1.06 (m, 1H). MS (m/z) 392.2 (M + H)⁺ | 1-cyclohexyl-3- (6- methoxypyridin- 3-yl)-7- (trifluoromethyl)- 2,3- dihydroquinazolin- 4(1H)-one |
| 38 | 1-(4-fluoro-2,6- dimethylphenyl)-3- (6-oxo-1,6- dihydropyridin-3- yl)-7- (trifluoromethyl)- 2,3- dihydroquinazolin- 4(1H)-one | 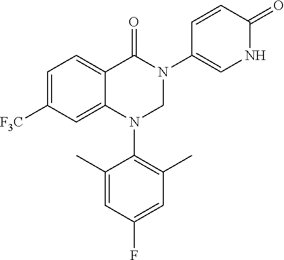 [SMILES: Cc1cc(F)cc(C)c1N1CN(c2ccc(=O)[nH]c2)C(=O)c2ccc(C(F)(F)F)cc21] | ¹H NMR (400 MHz, DMSO-d₆) δ: 11.74 (br. s., 1H), 8.07 (d, J = 8.0 Hz, 1H), 7.56-7.45 (m, 2H), 7.21-7.11 (m, 3H), 6.37 (d, J = 9.6 Hz, 1H), 6.23 (s, 1H), 5.21 (s, 2H), 2.18 (s, 6H). MS (m/z) 431.9 (M + H)⁺ | 1-(4-fluoro-2,6- dimethylphenyl)- 3-(6- methoxypyridin- 3-yl)-7- (trifluoromethyl)- 2,3- dihydroquinazolin- 4(1H)-one |
| 39 | 1-(4-fluoro-2- methylphenyl)-2- methyl-3-(6-oxo- 1,6-dihydropyridin- 3-yl)-7- (trifluoromethyl)- 2,3- dihydroquinazolin- 4(1H)-one | 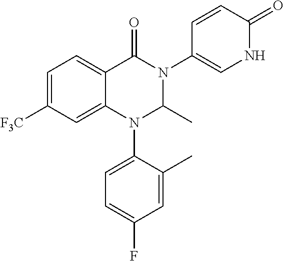 [SMILES: Cc1cc(F)ccc1N1c2cc(C(F)(F)F)ccc2C(=O)N(c2ccc(=O)[nH]c2)C1C] | ¹H NMR (400 MHz, DMSO-d₆) δ: 11.75 (br. s., 1 H), 8.10-8.04 (m, 1 H), 7.51-7.12 (m, 6 H), 6.60-6.37 (m, 2 H), 5.28- 5.22 (m, 1 H), 2.34 (br. s., 1 H), 2.23 (s, 2 H), 1.51 (d, J = 5.38 Hz, 2 H), 1.40 (br. s., 1 H). MS (m/z) 432.2 (M + H)⁺ | 1-(4-fluoro-2- methylphenyl)- 3-(6- methoxypyridin- 3-yl)-2-methyl- 7- (trifluoromethyl)- 2,3- dihydroquinazolin- 4(1H)-one |
| 40 | 1-(2-chloro-4- fluorophenyl)-3-(6- oxo-1,6- dihydropyridin-3- yl)-7- (trifluoromethyl)- 2,3- dihydroquinazolin- 4(1H)-one | 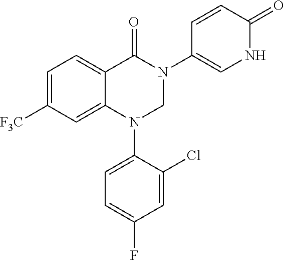 [SMILES: O=C1c2ccc(C(F)(F)F)cc2N(c2ccc(F)cc2Cl)CN1c1ccc(=O)[nH]c1] | ¹H NMR (400 MHz, DMSO-d₆) δ: 11.75 (br. s., 1H), 8.09 (d, J = 8.0 Hz, 1H), 7.73 (dd, J = 8.6, 2.9 Hz, 1H), 7.64 (dd, J = 8.9, 5.7 Hz, 1H), 7.53-7.51 (m, 1H), 7.48 (dd, J = 9.6, 2.9 Hz, 1H), 7.41 (td, J = 8.5, 2.9 Hz, 1H), 7.30 (dd, J = 8.1, 1.0 Hz, 1H), 6.52 (s, 1H), 6.36 (d, J = 9.6 Hz, 1H), 5.27 (br. s., 2H). MS (m/z) 437.9 (M + H)⁺ | 1-(2-chloro-4- fluorophenyl)- 3-(6- methoxypyridin- 3-yl)-7- (trifluoromethyl)- 2,3- dihydroquinazolin- 4(1H)-one |
| 41 | 1-(4-fluoro-2-(2- hydroxyeth- oxy)phenyl)-3- (6-oxo-1,6- dihydropyridin-3- yl)-7- (trifluoromethyl)- 2,3- dihydroquinazolin- 4(1H)-one | 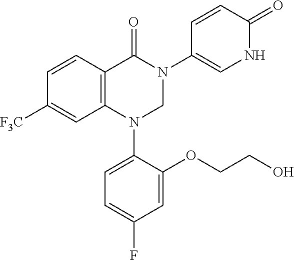 [SMILES: O=C1c2ccc(C(F)(F)F)cc2N(c2ccc(F)cc2OCCO)CN1c1ccc(=O)[nH]c1] | ¹H NMR (400 MHz, DMSO-d₆) δ: 11.71 (br. s., 1H), 8.05 (d, J = 8.1 Hz, 1H), 7.58-7.44 (m, 2H), 7.40 (dd, J = 8.7, 6.3 Hz, 1H), 7.29-7.13 (m, 2H), 6.89 (td, J = 8.4, 2.8 Hz, 1H), 6.57 (s, 1H), 6.35 (d, J = 9.5 Hz, 1H), 5.20 (s, 2H), 4.78 (t, J = 5.4 Hz, 1H), 4.05 (t, J = 4.9 Hz, 2H), 3.56 (q, J = 5.1 Hz, 2H). MS (m/z) 464.0 (M + H)⁺ | 1-(4-fluoro-2- (2- methoxyeth- oxy)phenyl)-3-(6- methoxypyridin- 3-yl)-7- (trifluoromethyl)- 2,3- dihydroquinazolin- 4(1H)-one |
| 42 | 1-(2,4- difluorophenyl)-3- (6-oxo-1,6- dihydropyridin-3- yl)-7- (trifluoromethyl)- 2,3- dihydroquinazolin- 4(1H)-one | 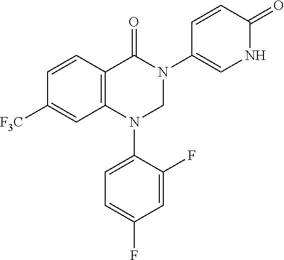 [SMILES: O=C1c2ccc(C(F)(F)F)cc2N(c2ccc(F)cc2F)CN1c1ccc(=O)[nH]c1] | ¹H NMR (400 MHz, DMSO-d₆) δ: 11.74 (br. s., 1H), 8.09 (d, J = 8.0 Hz, 1H), 7.66-7.42 (m, 4H), 7.33 (dd, J = 8.1, 1.0 Hz, 1H), 7.29-7.19 (m, 1H), 6.71 (s, 1H), 6.36 (d, J = 9.6 Hz, 1H), 5.29 (s, 2H). MS (m/z) 422.0 (M + H)⁺ | 1-(2,4- difluorophenyl)- 3-(6- methoxypyridin- 3-yl)-7- (trifluoromethyl)- 2,3- dihydroquinazolin- 4(1H)-one |
| 43 | 1-(2-ethyl-4- fluorophenyl)-3-(6- oxo-1,6- dihydropyridin-3- yl)-7- (trifluoromethyl)- 2,3- dihydroquinazolin- 4(1H)-one | 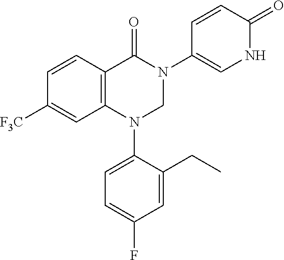 [SMILES: CCc1cc(F)ccc1N1CN(c2ccc(=O)[nH]c2)C(=O)c2ccc(C(F)(F)F)cc21] | ¹H NMR (400 MHz, DMSO-d₆) δ: 11.73 (br. s., 1H), 8.07 (d, J = 7.9 Hz, 1H), 7.56-7.45 (m, 2H), 7.42 (dd, J = 8.8, 5.5 Hz, 1H), 7.34 (dd, J = 9.9/3.0 Hz, 1H), 7.25- 7.18 (m, 2H), 6.40-6.31 (m, 2H), 5.43 (d, J = 9.8 Hz, 1H), 4.97 (d, J = 9.9 Hz, 1H), 2.63-2.54 (m, 2H), 1.13 (t, J = 7.5 Hz, 3H). MS (m/z) 432.0 (M + H)⁺ | 1-(2-ethyl-4- fluorophenyl)- 3-(6- methoxypyridin- 3-yl)-7- (trifluoromethyl)- 2,3- dihydroquinazolin- 4(1H)-one |
| 44 | 8-chloro-1-(4- fluoro-2- methylphenyl)-3- (6-oxo-1,6- dihydropyridin-3- yl)-7- (trifluoromethyl)- 2,3- dihydroquinazolin- 4(1H)-one | 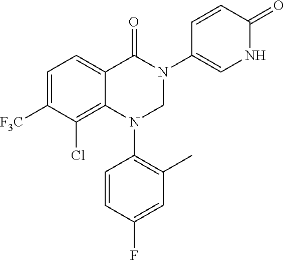 [SMILES: Cc1cc(F)ccc1N1CN(c2ccc(=O)[nH]c2)C(=O)c2ccc(C(F)(F)F)c(Cl)c21] | ¹H NMR (400 MHz, DMSO-d₆) δ: 11.66 (br. s., 1H), 8.17 (d, J = 8.1 Hz, 1H), 7.78 (d, J = 8.4 Hz, 1H), 7.31-7.19 (m, 2H), 7.15 (dd, J = 9.6, 2.9 Hz, 1H), 6.93 (td, J = 8.5, 3.1 Hz, 1H), 6.72 (dd, J = 8.7, 5.3 Hz, 1H), 6.31 (d, J = 9.8 Hz, 1H), 5.49 (d, J = 12.6 Hz, 1H), 4.75 (d, J = 12.6 Hz, 1H), 2.48 (s, 3H). MS (m/z) 452.0 (M + H)⁺ | 8-chloro-1-(4- fluoro-2- methylphenyl)- 3-(6- methoxypyridin- 3-yl)-7- (trifluoromethyl)- 2,3- dihydroquinazolin- 4(1H)-one |
| 45 | 1-(4-fluoro-2- methylphenyl)-8- methyl-3-(6-oxo- 1,6-dihydropyridin- 3-yl)-7- (trifluoromethyl)- 2,3- dihydroquinazolin- 4(1H)-one | 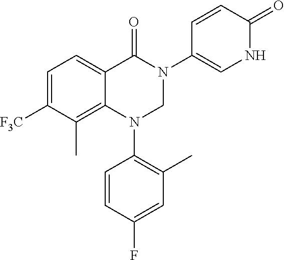 [SMILES: Cc1cc(F)ccc1N1CN(c2ccc(=O)[nH]c2)C(=O)c2ccc(C(F)(F)F)c(C)c21] | ¹H NMR (400 MHz, CDCl₃) δ: 12.75 (br. s., 1H), 8.16 (d, J = 8.4 Hz, 1H), 7.62 (d, J = 8.3 Hz, 1H), 7.21-7.04 (m, 3H), 6.80 (td, J = 8.1, 2.6 Hz, 1H), 6.64-6.48 (m, 2H), 5.38 (d, J = 12.4 Hz, 1H), 4.58 (d, J = 12.3 Hz, 1H), 2.53 (s, 3H), 2.00 (s, 3H). MS (m/z) 432.0 (M + H)⁺ | 1-(4-f1uoro-2- methylphenyl)- 3-(6- methoxypyridin- 3-yl)-8-methyl- 7- (trifluoromethyl)- 2,3- dihydroquinazolin- 4(1H)-one |
| 46 | 3-(2-methyl-6-oxo- 1,6-dihydropyridin- 3-yl)-1-(2- methylpyridin-3- yl)-7- (trifluoromethyl)- 2,3- dihydroquinazolin- 4(1H)-one | 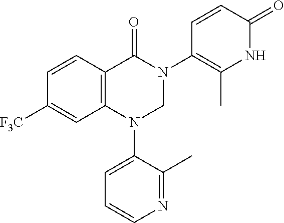 [SMILES: Cc1ncccc1N1CN(c2ccc(=O)[nH]c2C)C(=O)c2ccc(C(F)(F)F)cc21] | ¹H NMR (400 MHz, DMSO-d₆) δ: 11.79 (br. s., 1H), 8.50 (dd, J = 4.8, 1.4 Hz, 1H), 8.10 (d, J = 8.0 Hz, 1H), 7.76 (d, J = 7.6 Hz, 1H), 7.39 (d, J = 9.4 Hz, 2H), 7.30 (dd, J = 8.1, 0.9 Hz, 1H), 6.44 (br. s., 1H), 6.20 (d, J = 9.5 Hz, 1H), 5.57-4.76 (m, 2H), 2.42 (s, 3H), 2.09 (br. s., 3H). MS (m/z) 415.0 (M + H)⁺ | 3-(6-methoxy- 2- methylpyridin- 3-yl)-1-(2- methylpyridin- 3-yl)-7- (trifluoromethyl)- 2,3- dihydroquinazolin- 4(1H)-one |
| 47 | 1-(4-fluoro-2- isopropylphenyl)- 3-(2-methyl-6-oxo- 1,6-dihydropyridin- 3-yl)-7- (trifluoromethyl)- 2,3- dihydroquinazolin- 4(1H)-one | 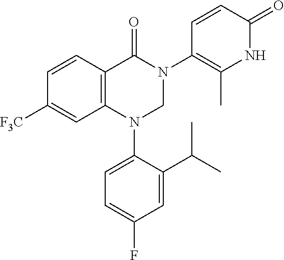 [SMILES: Cc1[nH]c(=O)ccc1N1CN(c2ccc(F)cc2C(C)C)c2cc(C(F)(F)F)ccc2C1=O] | ¹H NMR (400 MHz, DMSO-d₆) δ: 11.80 (br. s., 1H), 8.06 (d, J = 7.8 Hz, 1H), 7.46-7.36 (m, 3H), 7.26-7.18 (m, 2H), 6.34-6.25 (m, 1H), 6.23- 6.17 (m, 1H), 5.56 (d, J = 9.6 Hz, 0.6H), 5.25 (d, J = 10.1 Hz, 0.4H), 5.00 (d, J = 10.4 Hz, 0.4H), 4.72 (d, J = 9.4 Hz, 0.6H), 3.20-3.02 (m, 1H), 2.13 (s, 3H), 1.19 (m, 3H), 1.17 (m, 3H). MS (m/z) 460.1 (M + H)⁺ | 1-(4-fluoro-2- isopropylphenyl)- 3-(6- methoxy-2- methylpyridin- 3-yl)-7- (trifluoromethyl)- 2,3- dihydroquinazolin- 4(1H)-one |
| 48 | 6-chloro-1-(4- fluoro-2- methylphenyl)-3- (2-methyl-6-oxo- 1,6-dihydropyridin- 3-yl)-2,3- dihydroquinazolin- 4(1H)-one | 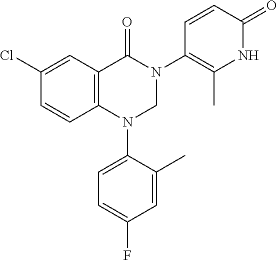 [SMILES: Cc1cc(F)ccc1N1CN(c2ccc(=O)[nH]c2C)C(=O)c2cc(Cl)ccc21] | ¹H NMR (400 MHz, DMSO-d₆) δ: 11.34 (br. s., 1H), 7.82 (s, 1H), 7.31-7.32 (m, 3H), 7.22 (dd, J = 9.60, 3.20 Hz, 1H), 7.08-7.09 (m, 1H), 6.27 (d, J = 8.80 Hz, 1H), 6.18 (d, J = 9.60 Hz, 1H), 5.03 (br. s., 2H), 2.24 (s, 3H), 2.12 (s, 3H). MS (m/z) 398.0 (M + H)⁺ | 6-chloro-1-(4- fluoro-2- methylphenyl)- 3-(6-methoxy- 2- methylpyridin- 3-yl)-2,3- dihydroquinazolin- 4(1H)-one |
| 49 | 1-(4-fluorophenyl)- 3-(2-methyl-6-oxo- 1,6-dihydropyridin- 3-yl)-2,3- dihydroquinazolin- 4(1H)-one | 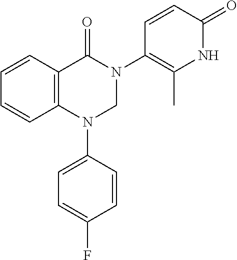 [SMILES: Cc1[nH]c(=O)ccc1N1CN(c2ccc(F)cc2)c2ccccc2C1=O] | ¹H NMR (400 MHz, DMSO-d₆) δ: 11.74 (s, 1H), 7.92 (dd, J = 7.80, 1.60 Hz, 1H), 7.48-7.44 (m, 1H), 7.33-7.21 (m, 5H), 7.24-7.09 (m, 1H), 6.89 (d, J = 8.00 Hz, 1H), 6.17 (d, J = 9.60 Hz, 1H), 5.28 (d, J = 11.20 Hz, 1H), 5.06 (d, J = 11.20 Hz, 1H), 1.91 (s, 3H). MS (m/z) 350.0 (M + H)⁺ | 1-(4- fluorophenyl)- 3-(6-methoxy- 2- methylpyridin- 3-yl)-2,3- dihydroquinazolin- 4(1H)-one |
| 50 | 3-(2-methyl-6-oxo- 1,6-dihydropyridin- 3-yl)-1-(o-tolyl)- 2,3- dihydroquinazolin- 4(1H)-one | 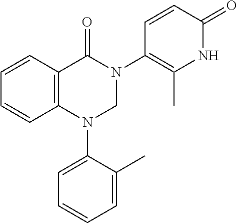 [SMILES: Cc1ccccc1N1CN(c2ccc(=O)[nH]c2C)C(=O)c2ccccc21] | ¹H NMR (400 MHz, DMSO-d₆) δ: 11.75 (s, 1H), 7.87 (dd, J = 8.00, 1.60 Hz, 1H), 7.43-7.18 (m, 6H), 6.94 (t, J = 7.20 Hz, 1H), 6.28-6.16 (m, 2H), 5.20-4.60 (m, 2H), 2.22 (s, 3H), 2.08 (s, 3H). MS (m/z) 346.2 (M + H). | 3-(6-methoxy- 2- methylpyridin- 3-yl)-1-(o- tolyl)-2,3- dihydroquinazolin- 4(1H)-one |
| 51 | 1-(4-f1uoro-2- methylphenyl)-6- methyl-3-(2- methyl-6-oxo-1,6- dihydropyridin-3- yl)-2,3- dihydroquinazolin- 4(1H)-one | 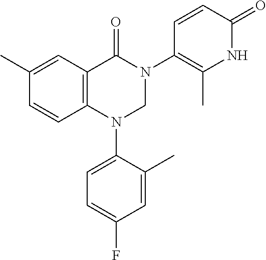 [SMILES: Cc1ccc2c(c1)C(=O)N(c1ccc(=O)[nH]c1C)CN2c1ccc(F)cc1C] | ¹H NMR (400 MHz, DMSO-d₆) δ: 11.38 (s, 1H), 7.70 (d, J = 2.00 Hz, 1H), 7.28 (d, J = 9.20 Hz, 1H), 7.19-7.19 (m, 3H), 7.08 (q, J = 3.20 Hz, 1H), 6.18 (t, J = 9.20 Hz, 2H), 4.60-5.50 (m, 2H), 2.27 (s, 3H), 2.24 (s, 3H), 2.09 (s, 3H). MS (m/z) 378.2 (M + H)⁺ | 1-(4-fluoro-2- methylphenyl)- 3-(6-methoxy- 2- methylpyridin- 3-yl)-6-methyl- 2,3- dihydroquinazolin- 4(1H)-one |
| 52 | 1-(4-fluoro-2- methylphenyl)-3- (2-methyl-6-oxo- 1,6-dihydropyridin- 3-yl)-2,3- dihydroquinazolin- 4(1H)-one | 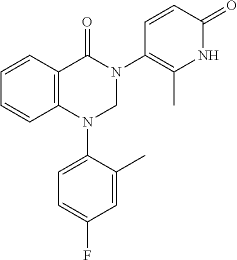 [SMILES: Cc1cc(F)ccc1N1CN(c2ccc(=O)[nH]c2C)C(=O)c2ccccc21] | ¹H NMR (400 MHz, DMSO-d₆, 80° C.) δ: 11.35 (s, 1H), 7.89 (dd, J = 1.60, 7.60 Hz, 1H), 7.35-7.20 (m, 4H), 7.10 (dt, J = 12.40, 3.20 Hz, 1H) 6.94 (t, J = 7.20 Hz, 1H), 6.25 (d, J = 8.00 Hz, 1H), 6.18 (d, J = 9.60 Hz, 1H), 4.97 (br. s., 2H), 2.25 (s, 3H), 2.11 (s, 3H). MS (m/z) 364.0 (M + H)⁺ | 1-(4-fluoro-2- methylphenyl)- 3-(6-methoxy- 2- methylpyridin- 3-yl)-2,3- dihydroquinazolin- 4(1H)-one |
| 53 | 1-(4-fluoro-2- methylphenyl)-3- (6-methyl-2-oxo- 1,2-dihydropyridin- 4-yl)-7- (trifluoromethyl)- 2,3- dihydroquinazolin- 4(1H)-one | 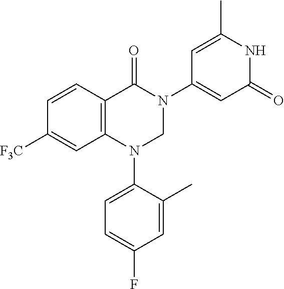 [SMILES: Cc1cc(N2CN(c3ccc(F)cc3C)c3cc(C(F)(F)F)ccc3C2=O)cc(=O)[nH]1] | ¹H NMR (400 MHz, DMSO-d₆, 80° C.) δ: 11.20 (s, 1H), 8.13 (d, J = 8.00 Hz, 1H), 7.37 (dd, J = 8.60, 5.20 Hz, 1H), 7.28-7.23 (m, 2H), 7.16 (dt, J = 8.40, 2.80 Hz, 1H), 6.45 (s, 1H), 6.18 (s, 1H), 6.11 (d, J = 1.60 Hz, 1H), 5.27 (s, 2H), 2.22 (s, 3H), 2.17 (s, 3H). MS (m/z) 432.0 (M + H)⁺ | 1-(4-fluoro-2- methylphenyl)- 3-(2-methoxy- 6- methylpyridin- 4-yl)-7- (trifluoromethyl)- 2,3- dihydroquinazolin- 4(1H)-one |
| 54 | 1-(4-fluoro-2- methylphenyl)-3- (5-methyl-2-oxo- 1,2-dihydropyridin- 4-yl)-7- (trifluoromethyl)- 2,3- dihydroquinazolin- 4(1H)-one | 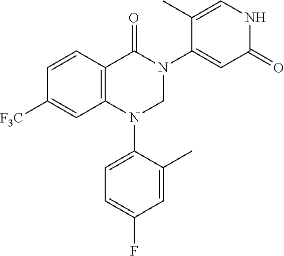 [SMILES: Cc1c[nH]c(=O)cc1N1CN(c2ccc(F)cc2C)c2cc(C(F)(F)F)ccc2C1=O] | ¹H NMR (400 MHz, DMSO-d₆, 80° C.) δ: 11.26 (br. s., 1H), 8.11 (d, J = 8.00 Hz, 1H), 7.36 (dd, J = 8.60, 5.20 Hz, 1H), 7.28-7.22 (m, 3H), 7.15 (dt, J = 8.40, 2.80 Hz, 1H), 6.45 (s, 1H), 6.31 (s, 1H), 5.20 (s, 2H), 2.24 (s, 3H), 1.91 (s, 3H). MS (m/z) 432.2 (M + H)⁺ | 1-(4-fluoro-2- methylphenyl)- 3-(2-methoxy- 5- methylpyridin- 4-yl)-7- (trifluoromethyl)- 2,3- dihydroquinazolin- 4(1H)-one |
| 55 | 1-(4-fluoro-2- methylphenyl)-3- (3-methyl-2-oxo- 1,2-dihydropyridin- 4-yl)-7- (trifluoromethyl)- 2,3- dihydroquinazolin- 4(1H)-one | 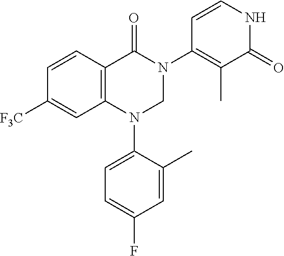 [SMILES: Cc1cc(F)ccc1N1CN(c2cc[nH]c(=O)c2C)C(=O)c2ccc(C(F)(F)F)cc21] | ¹H NMR (400 MHz, DMSO-d₆, 80° C.) δ: 8.16 (s, 1H), 8.10 (d, J = 8.00 Hz, 1H), 7.36-7.33 (m, 1H), 7.26-7.22 (m, 3H), 7.14 (dt, J = 8.60, 3.20 Hz, 1H), 6.43 (s, 1H), 6.15 (d, J = 7.20 Hz, 1H), 5.14, (br. s., 2H), 2.22 (s, 3H), 1.87 (s, 3H). MS (m/z) 432.0 (M + H)⁺ | 1-(4-fluoro-2- methylphenyl)- 3-(2-methoxy- 3- methylpyridin- 4-yl)-7- (trifluoromethyl)- 2,3- dihydroquinazolin- 4(1H)-one |
| 56 | 1-(4-fluoro-2- methylphenyl)-3- (2-methyl-6-oxo- 1,6-dihydropyridin- 3-yl)-7- (trifluoromethoxy)- 2,3- dihydroquinazolin- 4(1H)-one | 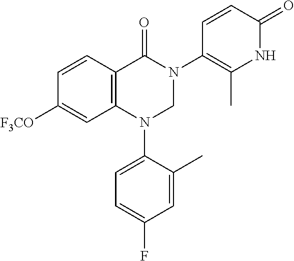 [SMILES: Cc1cc(F)ccc1N1CN(c2ccc(=O)[nH]c2C)C(=O)c2ccc(OC(F)(F)F)cc21] | ¹H NMR (400 MHz, DMSO-d₆, 80° C.) δ: 11.44 (s, 1H), 7.99 (d, J = 8.80 Hz, 1H), 7.38-7.32 (m, 2H), 7.26 (dd, J = 9.60, 2.80 Hz, 1H), 7.15 (dt, J = 12.40, 3.20 Hz, 1H), 6.83 (d, J = 8.00 Hz, 1H), 6.18 (d, J = 9.20 Hz, 1H), 6.01 (s, 1H), 5.03 (br. s., 2H), 2.25 (s, 3H), 2.13 (s, 3H). MS (m/z) 448.0 (M + H)⁺ | 1-(4-fluoro-2- methylphenyl)- 3-(6-methoxy- 2- methylpyridin- 3-yl)-7- (trifluorometh- oxy)-2,3- dihydroquinazolin- 4(1H)-one |
| 57 | 1-(4-fluoro-2- methylphenyl)-7- methyl-3-(2- methyl-6-oxo-1,6- dihydropyridin-3- yl)-2,3- dihydroquinazolin- 4(1H)-one | 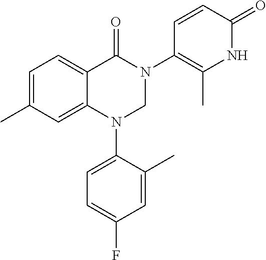 [SMILES: Cc1ccc2c(c1)N(c1ccc(F)cc1C)CN(c1ccc(=O)[nH]c1C)C2=O] | ¹H NMR (400 MHz, DMSO-d₆, 80° C.) δ: 11.39 (br. s., 1H), 7.77 (d, J = 8.00 Hz, 1H), 7.29 (d, J = 9.60 Hz, 1H), 7.26-7.20 (m, 2H), 7.12-7.07 (td, J = 2.80, 8.40 Hz, 1H), 6.76 (d, J = 7.60 Hz, 1H), 6.17 (d, J = 9.60 Hz, 1H), 6.06 (s, 1H), 5.21-4.82 (br. s., 2H) 2.25 (s, 3H), 2.20 (s, 3H), 2.10 (s, 3H). MS (m/z) 378.1 (M + H)⁺ | 1-(4-fluoro-2- methylphenyl)- 3-(6-methoxy- 2- methylpyridin- 3-yl)-7-methyl- 2,3- dihydroquinazolin- 4(1H)-one |
| 58 | 1-(4-fluoro-2- methylphenyl)-3- (3-methyl-5-oxo- 4,5- dihydropyrazin-2- yl)-7- (trifluoromethyl)- 2,3- dihydroquinazolin- 4(1H)-one | 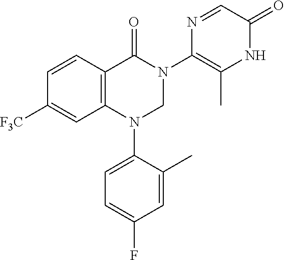 [SMILES: Cc1cc(F)ccc1N1CN(c2ncc(=O)[nH]c2C)C(=O)c2ccc(C(F)(F)F)cc21] | ¹H NMR (400 MHz, DMSO-d₆, as a mixture of rotamers) δ: 8.09 (d, J = 8.00 Hz, 1H), 7.58 (s, 1H), 7.49-7.39 (m, 1H), 7.32 (dd, J = 2.80, 9.60 Hz, 1H), 7.25-7.18 (m, 2H), 6.43-6.30 (m, 1H), 5.60-4.85 (m, 2H), 2.22 (s, 3H), 2.11 (s, 3H), one exchangeable proton not observed. MS (m/z) 433.1 (M + H)⁺ | 1-(4-fluoro-2- methylphenyl)- 3-(5-methoxy- 3- methylpyrazin- 2-yl)-7- (trifluoromethyl)- 2,3- dihydroquinazolin- 4(1H)-one |
| 59 | 6-(1-(4-fluoro-2- methylphenyl)-4- oxo-7- (trifluoromethyl)- 1,4- dihydroquinazolin- 3(2H)- yl)pyrimidine- 2,4(1H,3H)-dione | 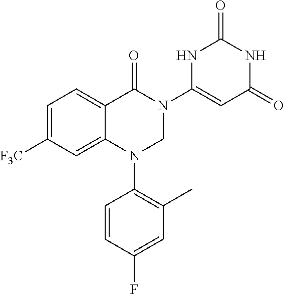 [SMILES: Cc1cc(F)ccc1N1CN(c2cc(=O)[nH]c(=O)[nH]2)C(=O)c2ccc(C(F)(F)F)cc21] | ¹H NMR (400 MHz, DMSO-d₆) δ: 11.28 (br s, 1H), 11.05 (br s, 1H), 8.12 (d, J = 8.1 Hz, 1H), 7.37 (dd, J = 8.7, 5.4 Hz, 1H), 7.32 (dd, J = 9.7, 2.9 Hz, 1H), 7.28 (dd, J = 8.3, 1.1 Hz, 1H), 7.20 (td, J = 8.5, 2.8 Hz, 1H), 6.45 (s, 1 H), 5.54 (d, J = 1.1 Hz, 1H), 5.36 (br s, 1H), 5.20 (br s, 1H), 2.22 (s, 3H). MS (m/z) 435.0 (M + H)⁺ | 3-(2,6- dimethoxy- pyrimidin-4-yl)-1- (4-fluoro-2- methylphenyl)- 7- (trifluoromethyl)- 2,3- dihydroquinazolin- 4(1H)-one |
| 60 | 1-(2-ethyl-4- fluorophenyl)-3-(2- methyl-6-oxo-1,6- dihydropyridin-3- yl)-6- (trifluoromethyl)- 2,3- dihydroquinazolin- 4(1H)-one | 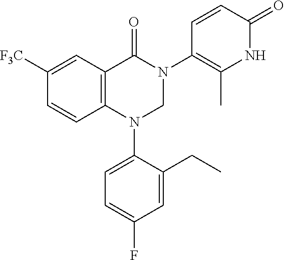 [SMILES: CCc1cc(F)ccc1N1CN(c2ccc(=O)[nH]c2C)C(=O)c2cc(C(F)(F)F)ccc21] | ¹H NMR (400 MHz, DMSO-d₆) δ: 11.80 (brs, 1H), 8.07 (d, J = 2.0 Hz, 1H), 7.64 (dd, J = 8.8, 2.1 Hz, 1H), 7.45-7.38 (m, 2H), 7.38-7.31 (m, 1H), 7.26-7.19 (m, 1H), 6.34-6.28 (m, 1H), 6.21 (d, J = 9.6 Hz, 1H), 5.55 (d, J = 9.5 Hz, 0.6H), 5.29 (d, J = 10.0 Hz, 0.4H), 4.98 (d, J = 10.0 Hz, 0.4H), 4.73 (d, J = 9.4 Hz, 0.6H), 2.65- 2.54 (m, 2H), 2.12 (s, 3H), 1.15 (q, J = 7.5 Hz, | 1-(2-ethyl-4- fluorophenyl)- 3-(6-methoxy- 2- methylpyridin- 3-yl)-6- (trifluoromethyl)- 2,3- dihydroquinazolin- 4(1H)-one |
| | | | 3H). | |
| | | | MS (m/z) 446.2 (M + H)⁺ | |
| 61 | 6-chloro-1-(4- fluoro-2- methylphenyl)-3- (4-methyl-2-oxo- 1,2- dihydropyrimidin- 5-yl)-2,3- dihydroquinazolin- 4(1H)-one | 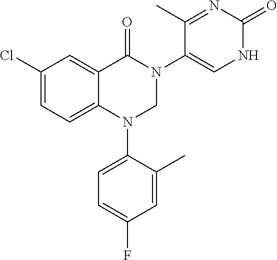 [SMILES: Cc1cc(F)ccc1N1CN(c2c[nH]c(=O)nc2C)C(=O)c2cc(Cl)ccc21] | ¹H NMR (400 MHz, CHLOROFORM-d,) δ: 8.26 (br s, 1H), 8.04 (d, J = 2.4 Hz, 1H), 7.32-7.25 (m, 2H), 7.16-7.06 (m, 2H), 7.05-6.96 (m, 1H), 6.31 (br d, J = 6.4 Hz, 1H), 5.45-4.56 (m, 2H), 2.41 (br s, 3H), 2.30 (s, 3H). MS (m/z) 399.0 (M + H)⁺ | 6-chloro-1-(4- fluoro-2- methylphenyl)- 3-(2-methoxy- 4- methylpyrimidin- 5-yl)-2,3- dihydroquinazolin- 4(1H)-one |
| 62 | 3-(2,4-dimethyl-6- oxo-1,6- dihydropyridin-3- yl)-1-(4-fluoro-2- isopropylphenyl)- 7-(trifluoromethyl)- 2,3- dihydroquinazolin- 4(1H)-one | 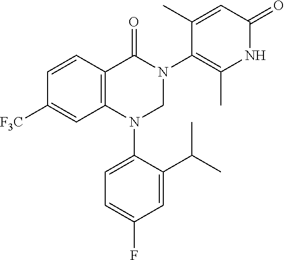 [SMILES: Cc1cc(=O)[nH]c(C)c1N1CN(c2ccc(F)cc2C(C)C)c2cc(C(F)(F)F)ccc2C1=O] | ¹HNMR (400 MHz, DMSO-d₆) δ: 11.64 (br s, 1H), 8.06 (d, J = 8.0 Hz, 1H), 7.48-7.38 (m, 2H), 7.26-7.19 (m, 2H), 6.27 (s, 1H), 6.10 (d, J = 4.3 Hz, 1H), 5.52 (dd, J = 9.8, 1.7 Hz, 1H), 4.77 (dd, J = 12.7, 9.7 Hz, 1H), 3.21-3.10 (m, 1H), 2.14 (d, J = 1.6 Hz, 3H), 2.06 (dd, J = 9.6, 0.6 Hz, 3H), 1.17 (dd, J = 6.9, 2.9 Hz, 3H), 1.10 (d, J = 6.9 Hz, 3H) MS (m/z) 474.2 (M + H)⁺ | 1-(4-fluoro-2- isopropylphenyl)- 3-(6- methoxy-2,4- dimethylpyridin- 3-yl)-7- (trifluoromethyl)- 2,3- dihydroquinazolin- 4(1H)-one |
| 63 | 1-(4-fluoro-2- isopropylphenyl)- 3-(6-methoxy-2,4- dimethylpyridin-3- yl)-7- (trifluoromethyl)- 2,3- dihydroquinazolin- 4(1H)-one | 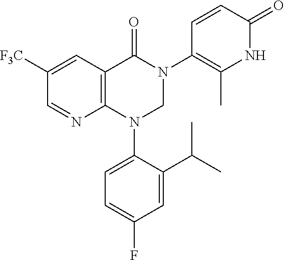 [SMILES: Cc1[nH]c(=O)ccc1N1CN(c2ccc(F)cc2C(C)C)c2ncc(C(F)(F)F)cc2C1=O] | ¹H NMR (400 MHz, DMSO-d₆, 24° C.): δ = 11.83 (br s, 1H), 8.58 (s, 1H), 8.29 (d, J = 2.4 Hz, 1H), 7.46-7.29 (m, 3H), 7.15 (td, J = 8.4, 2.9 Hz, 1H), 6.22 (dd, J = 9.4, 4.3 Hz, 1H), 5.62 (d, J = 9.5 Hz, 0.6H), 5.34 (d, J = 10.0 Hz, 0.4H), 5.09 (d, J = 10.0 Hz, 0.4H), 4.85 (d, J = 9.6 Hz, 0.6H), 3.15-2.98 (m, 1H), 2.20- 2.10 (m, 3H), 1.18- 1.06 (m, 6H) MS (m/z) 461.2 (M + H)⁺ | 1-(4-fluoro-2- isopropylphenyl)- 3-(6- methoxy-2- methylpyridin- 3-yl)-6- (trifluoromethyl)- 2,3- dihydropyrido[2,3- d]pyrimidin- 4(1H)-one |
| 64 | 3-(2-ethyl-6-oxo- 1,6-dihydropyridin- 3-yl)-1-(4-fluoro-2- methylphenyl)-6- (trifluoromethyl)- 2,3- dihydroquinazolin- 4(1H)-one | 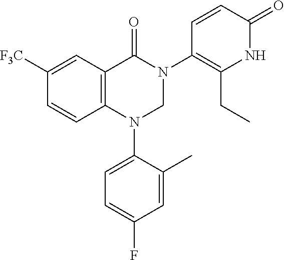 [SMILES: CCc1[nH]c(=O)ccc1N1CN(c2ccc(F)cc2C)c2ccc(C(F)(F)F)cc2C1=O] | ¹HNMR (400 MHz, DMSO-d₆): δ = 11.77 (br s, 1H), 8.08 (d, J = 2.0 Hz, 1H), 7.65 (dd, J = 8.8, 2.1 Hz, 1H), 7.45- 7.29 (m, 3H), 7.27-7.12 (m, 1H), 6.42-6.28 (m, 1H), 6.22 (d, J = 7.9 Hz, 1H), 5.58 (d, J = 9.5 Hz, 0.6H), 5.20-5.05 (m, 0.8H), 4.72 (d, J = 9.5 Hz, 0.6H), 2.46-2.36 (m, 2H), 2.26-2.16 (m, 3H), 1.07 (t, J = 7.6 Hz, 3H) MS (m/z) 446.2 (M + H)⁺ | 3-(2-ethyl-6- methoxypyridin- 3-yl)-1-(4- fluoro-2- methylphenyl)- 6- (trifluoromethyl)- 2,3- dihydroquinazolin- 4(1H)-one |
| 65 | 7-(dimethylamino)- 1-(4-fluoro-2- methylphenyl)-3- (2-methyl-6-oxo- 1,6-dihydropyridin- 3-yl)-2,3- dihydroquinazolin- 4(1H)-one | 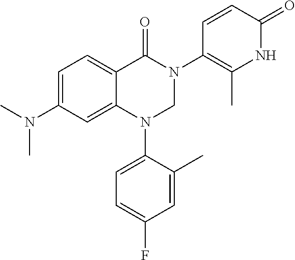 [SMILES: Cc1cc(F)ccc1N1CN(c2ccc(=O)[nH]c2C)C(=O)c2ccc(N(C)C)cc21] | ¹H NMR (400 MHz, DMSO-d₆, 80° C.): δ 11.31 (bs, 1H), 7.70 (d, J = 8.80 Hz, 1H), 7.28-7.20 (m, 3H), 7.10 (td, J = 8.40, 2.40 Hz, 1H), 6.36 (dd, J = 8.80, 2.00 Hz, 1H), 6.16 (d, J = 9.60 Hz, 1H), 5.36 (s, 1H), 5.20- 4.62 (br s, 2H), 2.82 (s, 6H), 2.27 (s, 3H), 2.10 (s, 3H). MS (m/z) 407.2 (M + H)⁺ | 7- (dimethylamino)- 1-(4-fluoro-2- methylphenyl)- 3-(6-methoxy- 2- methylpyridin- 3-yl)-2,3- dihydroquinazolin- 4(1H)-one |
| 66 | 7-chloro-1-(4- fluoro-2- methylphenyl)-3- (2-methyl-6-oxo- 1,6-dihydropyridin- 3-yl)-2,3- dihydroquinazolin- 4(1H)-one | 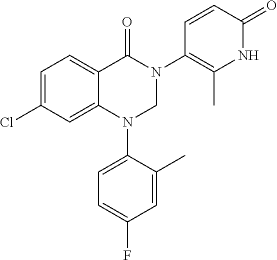 [SMILES: Cc1cc(F)ccc1N1CN(c2ccc(=O)[nH]c2C)C(=O)c2ccc(Cl)cc21] | ¹H NMR 400 MHz, DMSO-d₆: δ 11.4 (br s, 1H), 7.87 (d, J = 8.40 Hz, 1H), 7.36-7.31 (m, 2H), 7.25 (dd, J = 9.60, 2.80 Hz, 1H), 7.14 (dt, J = 12.40, 3.20 Hz, 1H), 6.94 (dd, J = 8.20, 1.60 Hz, 1H), 6.19-6.16 (m, 2H), 5.0 (br s, 2H), 2.25 (s, 3H), 2.12 (s, 3H). MS (m/z) 398.0 (M + H)⁺ | 7-chloro-1-(4- fluoro-2- methylphenyl)- 3-(6-methoxy- 2- methylpyridin- 3-yl)-2,3- dihydroquinazolin- 4(1H)-one |
| 67 | 1-(4-fluoro-2- methylphenyl)-3- (2-methyl-6-oxo- 1,6-dihydropyridin- 3-yl)-7- (methylamino)-2,3- dihydroquinazolin- 4(1H)-one | 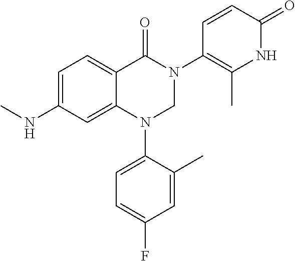 [SMILES: CNc1ccc2c(c1)N(c1ccc(F)cc1C)CN(c1ccc(=O)[nH]c1C)C2=O] | ¹H NMR (400 MHz, DMSO-d₆, 80° C.): δ 11.28 (br s, 1H), 7.63 (d, J = 8.40 Hz, 1H), 7.28- 7.20 (m, 3H), 7.12-7.08 (m, 1H), 6.19-6.14 (m, 3H), 6.11-6.07 (m, 1H), 5.29 (s, 1H), 5.23-4.55 (br s, 1H), 2.68 (d, J = 3.60 Hz, 3H), 2.34 (s, 3H), 2.34 (s, 3H). MS (m/z) 393.2 (M + H)⁺ | 1-(4-fluoro-2- methylphenyl)- 3-(6-methoxy- 2- methylpyridin- 3-yl)-7- (methylamino)- 2,3- dihydroquinazolin- 4(1H)-one |
| 68 | 6-chloro-1-(2- ethoxy-4- fluorophenyl)-3-(2- methyl-6-oxo-1,6- dihydropyridin-3- yl)-2,3- dihydroquinazolin- 4(1H)-one | 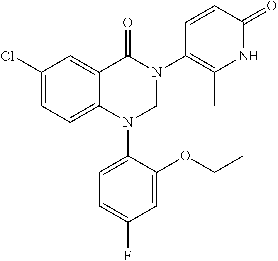 [SMILES: CCOc1cc(F)ccc1N1CN(c2ccc(=O)[nH]c2C)C(=O)c2cc(Cl)ccc21] | ¹H NMR 400 MHz, DMSO-d₆: δ 11.75 (s, 1H), 7.77 (d, J = 2.40 Hz, 1H), 7.39-7.29 (m, 3H), 7.10 (dd, J = 11.20, 2.80 Hz, 1H), 6.84 (dd, J = 8.40, 2.80 Hz, 1H), 6.42 (d, J = 8.80 Hz, 1H), 6.18 (d, J = 10.40 Hz, 1H), 5.21 (d, J = 10.40 Hz, 1H), 4.81 (d, J = 10.40 Hz, 1H), 4.05 (q, J = 6.80 Hz, 2H), 2.05 (s, 3H), 1.16 (t, J = 6.80 Hz, 3H). MS (m/z) 428.0 (M + H)⁺ | 6-chloro-1-(2- ethoxy-4- fluorophenyl)- 3-(6-methoxy- 2- methylpyridin- 3-yl)-2,3- dihydroquinazolin- 4(1H)-one |
| 69 | 1-(4-fluoro-2- isopropylphenyl)- 7-methyl-3-(2- methyl-6-oxo-1,6- dihydropyridin-3- yl)-2,3- dihydroquinazolin- 4(1H)-one | 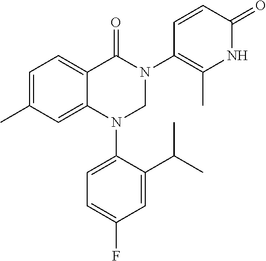 [SMILES: Cc1ccc2c(c1)N(c1ccc(F)cc1C(C)C)CN(c1ccc(=O)[nH]c1C)C2=O] | ¹H NMR (400 MHz, DMSO-d₆, 80° C.): δ 11.5- 11.3 (br s, 1H), 7.77 (d, J = 8.00 Hz, 1H), 7.32-7.27 (m, 3H), 7.13 (td, J = 8.40, 2.80 Hz, 1H), 6.75 (d, J = 7.20 Hz, 1H), 6.19 (d, J = 9.20 Hz, 1H), 6.01 (s, 1H), 5.40-5.10 (br s, 1H), 4.90-4.48 (br s, 1H), 3.27-3.18 (m, 1H), 2.19 (s, 3H), 2.13 (s, 3H), 1.18 (d, J = 6.80 Hz, 6H). MS (m/z) 406.2 (M + H)⁺ | 1-(4-fluoro-2- isopropylphenyl)- 3-(6- methoxy-2- methylpyridin- 3-yl)-7-methyl- 2,3- dihydroquinazolin- 4(1H)-one |
| 70 | 7-(difluoromethyl)- 6-fluoro-1-(4- fluoro-2- methylphenyl)-3- (2-methyl-6-oxo- 1,6-dihydropyridin- 3-yl)-2,3- dihydroquinazolin- 4(1H)-one | 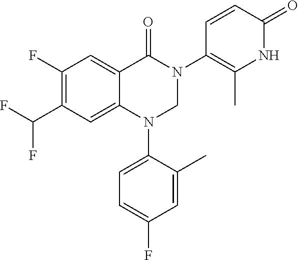 [SMILES: Cc1cc(F)ccc1N1CN(c2ccc(=O)[nH]c2C)C(=O)c2cc(F)c(C(F)F)cc21] | ¹H NMR 400 MHz, DMSO-d₆: δ 11.79 (s, 1H), 7.73 (d, J = 10.40 Hz, 1H), 7.37-7.26 (m, 3H), 7.21-6.99 (m, 2H), 6.62-6.32 (m, 1H), 6.19 (d, J = 9.60 Hz, 1H), 5.45-4.72 (m, 2H), 2.24 (s, 3H), 2.06 (br s, 3H). MS (m/z) 432.0 (M + H)⁺ | 7-(difluoromethyl)- 6-fluoro-1-(4- fluoro-2- methylphenyl)- 3-(6-methoxy- 2- methylpyridin- 3-yl)-2,3- dihydroquinazolin- 4(1H)-one |
| 71 | 6-fluoro-1-(4- fluoro-2- methylphenyl)-7- methyl-3-(2- methyl-6-oxo-1,6- dihydropyridin-3- yl)-2,3- dihydropyrido[2,3- d]pyrimidin-4(1H)- one | 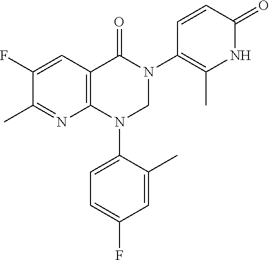 [SMILES: Cc1cc(F)ccc1N1CN(c2ccc(=O)[nH]c2C)C(=O)c2cc(F)c(C)nc21] | ¹H NMR 400 MHz, DMSO-d₆: δ 11.79 (s, 1H), 7.90 (d, J = 8.80 Hz, 1H), 7.37 (d, J = 9.60 Hz, 1H), 7.27 (dd, J = 8.60, 5.60 Hz, 1H), 7.20 (dd, J = 10.00, 3.20 Hz, 1H), 7.09 (td, J = 8.60, 3.20 Hz, 1H), 6.19 (d, J = 9.60 Hz, 1H), 5.53-4.72 (m, 2H), 2.25 (d, J = 3.20 Hz, 3H), 2.18 (s, 3H), 2.09 (s, 3H). MS (m/z) 397.2 (M + H)⁺ | 6-fluoro-1-(4- fluoro-2- methylphenyl)- 3-(6-methoxy- 2- methylpyridin- 3-yl)-7-methyl- 2,3- dihydropyrido[2,3- d]pyrimidin- 4(1H)-one |
| 72 | 6-chloro-1-(4- fluoro-2- methylphenyl)-7- methyl-3-(2- methyl-6-oxo-1,6- dihydropyridin-3- yl)-2,3- dihydropyrido[2,3- d]pyrimidin-4(1H)- one | 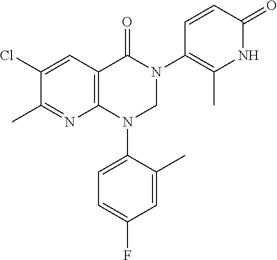 [SMILES: Cc1cc(F)ccc1N1CN(c2ccc(=O)[nH]c2C)C(=O)c2cc(Cl)c(C)nc21] | ¹H NMR 400 MHz, DMSO-d₆: δ 11.80 (s, 1H), 8.04 (s, 1H), 7.42- 7.32 (m, 1H), 7.30 (dd, J = 8.60, 5.20 Hz, 1H), 7.22 (dd, J = 10.00, 2.80 Hz, 1H), 7.10 (td, J = 8.40, 2.80 Hz, 1H), 6.19 (d, J = 9.20 Hz, 1H), 5.54-4.74 (m, 2H), 2.33 (s, 3H), 2.18 (s, 3H), 2.10 (s, 3H). MS (m/z) 413.1 (M + H)⁺ | 6-chloro-1-(4- fluoro-2- methylphenyl)- 3-(6-methoxy- 2- methylpyridin- 3-yl)-7-methyl- 2,3- dihydropyrido[2,3- d]pyrimidin- 4(1H)-one |
| 73 | 6,7-dichloro-1-(4- fluoro-2- methylphenyl)-3- (2-methyl-6-oxo- 1,6-dihydropyridin- 3-yl)-2,3- dihydropyrido[2,3- d]pyrimidin-4(1H)- one | 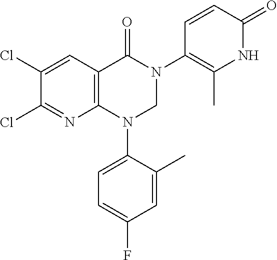 [SMILES: Cc1cc(F)ccc1N1CN(c2ccc(=O)[nH]c2C)C(=O)c2cc(Cl)c(Cl)nc21] | ¹H NMR 400 MHz, DMSO-d₆: δ 11.80 (br s, 1H), 8.24 (s, 1H), 7.46- 7.41 (m, 2H), 7.25 (dd, J = 9.80, 2.40 Hz, 1H), 7.16 (t, J = 5.60 Hz, 1H), 6.20 (d, J = 10.00 Hz, 1H), 5.53 (d, J = 9.60 Hz, 0.6H), 5.26 (d, J = 8.00 Hz, 0.4H), 5.12 (d, J = 11.60 Hz, 0.4H), 4.87 (d, J = 9.60 Hz, 0.6H), 2.21 (s, 3H), 2.11 (s, 3H). MS (m/z) 433.0 (M + H)⁺ | 6,7-dichloro-1- (4-fluoro-2- methylphenyl)- 3-(6-methoxy- 2- methylpyridin- 3-yl)-2,3- dihydropyrido[2,3- d]pyrimidin- 4(1H)-one |
| 74 | 6-chloro-5-fluoro- 1-(2-methyl-4- (trifluorometh- oxy)phenyl)-3-(2- methyl-6-oxo-1,6- dihydropyridin-3- yl)-2,3- dihydroquinazolin- 4(1H)-one | 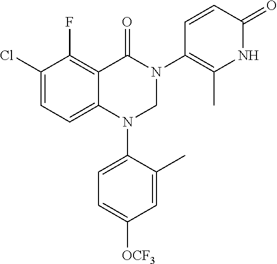 [SMILES: Cc1cc(OC(F)(F)F)ccc1N1CN(c2ccc(=O)[nH]c2C)C(=O)c2c1ccc(Cl)c2F] | ¹H NMR 400 MHz, DMSO-d₆: δ 11.76 (s, 1H), 7.51 (dd, J = 8.80, 7.60 Hz, 1H), 7.45 (s, 1H), 7.40-7.31 (m, 3H), 6.28-6.04 (m, 2H), 5.42- 4.78 (m, 2H), 2.24 (s, 3H), 2.07 (s, 3H). MS (m/z) 482.0 (M + H)⁺ | 6-chloro-5- fluoro-3-(6- methoxy-2- methylpyridin- 3-yl)-1-(2- methyl-4- (trifluorometh- oxy)phenyl)-2,3- dihydroquinazolin- 4(1H)-one |
| 75 | 1-(4-fluoro-2- methylphenyl)-3- (2-methyl-6-oxo- 1,6-dihydropyridin- 3-yl)-4-oxo- 1,2,3,4- tetrahydroquin- azoline-6- carbonitrile | 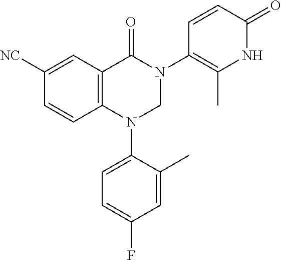 [SMILES: Cc1cc(F)ccc1N1CN(c2ccc(=O)[nH]c2C)C(=O)c2cc(C#N)ccc21] | ¹H NMR (400 MHz, DMSO-d₆): δ 11.80 (s, 1H), 8.16 (d, J = 2.00 Hz, 1H), 7.69 (dd, J = 8.60, 2.40 Hz, 1H), 7.32-7.34 (m, 3H), 7.20-7.22 (m, 1H), 6.18-6.19 (m, 2H), 4.79-4.82 (m, 2H), 2.22 (s, 3H), 2.12 (s, 3H) MS (m/z) 389.0 (M + H)⁺ | 1-(4-fluoro-2- methylphenyl)- 3-(6-methoxy- 2- methylpyridin- 3-yl)-4-oxo- 1,2,3,4- tetrahydroquin- azoline-6- carbonitrile |
| 76 | 6-chloro-1-(4- fluoro-2- isopropylphenyl)- 3-(2-methyl-6-oxo- 1,6-dihydropyridin- 3-yl)-2,3- dihydroquinazolin- 4(1H)-one | 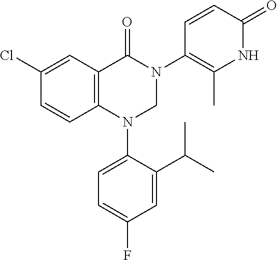 [SMILES: Cc1[nH]c(=O)ccc1N1CN(c2ccc(F)cc2C(C)C)c2ccc(Cl)cc2C1=O] | ¹H NMR (400 MHz, DMSO-d₆, 80° C): δ 11.46 (s, 1H), 7.81 (d, J = 2.40 Hz, 1H), 7.29-7.28 (m, 4H), 7.15 (dt, J = 12.00, 2.80 Hz, 1H), 6.21-6.19 (m, 2H), 5.36-4.75 (m, 2H), 3.21-3.18 (m, 1H), 2.14 (s, 3H), 1.18 (d, J = 4.00 Hz, 6H) MS (m/z) 426.0 (M + H)⁺ | 6-chloro-1-(4- fluoro-2- isopropylphenyl)- 3-(6- methoxy-2- methylpyridin- 3-yl)-2,3- dihydroquinazolin- 4(1H)-one |
| 77 | 1-(4-fluoro-3- hydroxy-2- methylphenyl)-3- (2-methyl-6-oxo- 1,6-dihydropyridin- 3-yl)-7- (trifluoromethyl)- 2,3- dihydroquinazolin- 4(1H)-one | 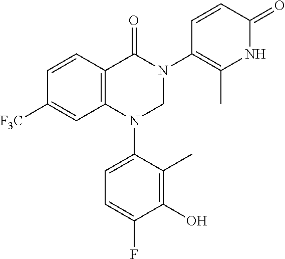 [SMILES: Cc1[nH]c(=O)ccc1N1CN(c2ccc(F)c(O)c2C)c2cc(C(F)(F)F)ccc2C1=O] | ¹H NMR (400 MHz, DMSO-d₆): δ 11.78 (s, 1H), 9.88 (s, 1H), 8.05 (d, J = 8.00 Hz, 1H), 7.37 (d, J = 9.60 Hz, 1H), 7.23-7.12 (m, 2H), 6.82- 6.74 (m, 1H), 6.47-6.35 (m, 1H), 6.19 (d, J = 9.20 Hz, 1H), 5.47-4.73 (m, 2H), 2.12-2.06 (m, 6H). MS (m/z) 448 (M + H)⁺ | 1-(4-fluoro-3- methoxy-2- methylphenyl)- 3-(6-methoxy- 2- methylpyridin- 3-yl)-7- (trifluoromethyl)- 2,3- dihydroquinazolin- 4(1H)-one |
| 78 | 6-chloro-5-fluoro- 1-(4-fluoro-2- methylphenyl)-3- (6-oxo-1,6- dihydropyridin-3- yl)-2,3- dihydroquinazolin- 4(1H)-one | 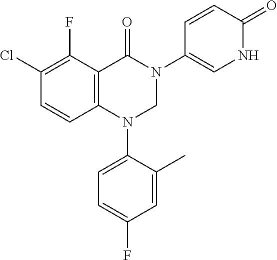 [SMILES: Cc1cc(F)ccc1N1CN(c2ccc(=O)[nH]c2)C(=O)c2c1ccc(Cl)c2F] | ¹H NMR 400 MHz, DMSO-d₆: δ 11.69 (s, 1H), 7.50-7.41 (m, 3H), 7.34-7.27 (m, 2H), 7.17- 7.12 (m, 1H), 6.34 (d, J = 9.60 Hz, 1H), 6.11 (dd, J = 9.00, 1.20 Hz, 1H), 5.23-4.95 (m, 2H), 2.21 (s, 3H). MS (m/z) 402.0 (M + H)⁺ | 6-chloro-5- fluoro-1-(4- fluoro-2- methylphenyl)- 3-(6- methoxypyridin- 3-yl)-2,3- dihydroquinazolin- 4(1H)-one |
| 79 | 6-chloro-5-fluoro- 1-(4-fluoro-2- methylphenyl)-3- (2-methyl-6-oxo- 1,6-dihydropyridin- 3-yl)-2,3- dihydroquinazolin- 4(1H)-one | 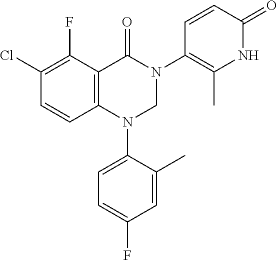 [SMILES: Cc1cc(F)ccc1N1CN(c2ccc(=O)[nH]c2C)C(=O)c2c1ccc(Cl)c2F] | ¹H NMR 400 MHz, DMSO-d₆: δ 11.76 (s, 1H), 7.49 (dd, J = 9.20, 7.60 Hz, 1H), 7.34-7.27 (m, 3H), 7.17-7.14 (m, 1H), 6.18 (d, J = 9.60 Hz, 1H), 6.18-6.02 (m, 1H), 5.33-4.79 (m, 2H), 2.21 (s, 3H), 2.08 (s, 3H). MS (m/z) 416.0 (M + H)⁺ | 6-chloro-5- fluoro-1-(4- fluoro-2- methylphenyl)- 3-(6-methoxy-2- methylpyridin- 3-yl)-2,3- dihydroquinazolin- 4(1H)-one |
| 80 | 1-(4-fluoro-2- methylphenyl)-3- (2-methyl-6-oxo- 1,6-dihydropyridin, 3-yl)-7- (methylsulfonyl)- 2,3- dihydroquinazolin- 4(1H)-one | 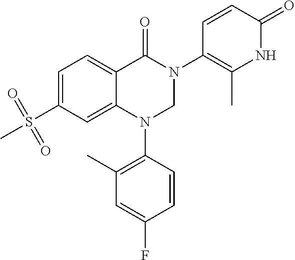 [SMILES: Cc1cc(F)ccc1N1CN(c2ccc(=O)[nH]c2C)C(=O)c2ccc(S(C)(=O)=O)cc21] | ¹H NMR (400 MHz, DMSO-d₆) δ: 11.79 (br s, 1H), 8.09 (d, J = 8.5 Hz, 1H) 7.47-7.30 (m, 4H), 7.21 (br s, 1H), 6.73- 6.58 (m, 1H), 6.20 (d, J = 9.5 Hz, 1H), 5.55-5.45 (m, 0.5H), 5.25-5.03 (m, 1H), 4.85-4.70 (m, 0.5H), 3.18 (s, 3H), 2.24 (s, 3H), 2.11 (br s, 3H) MS (m/z) 442.0 (M + H)⁺ | 1-(4-fluoro-2- methylphenyl)- 3-(6-methoxy- 2- methylpyridin- 3-yl)-7- (methylsulfonyl)- 2,3- dihydroquinazolin- 4(1H)-one |
| 81 | 6-chloro-1-(2- ethyl-4- fluorophenyl)-3-(2- methyl-6-oxo-1,6- dihydropyridin-3- yl)-2,3- dihydroquinazolin- 4(1H)-one | 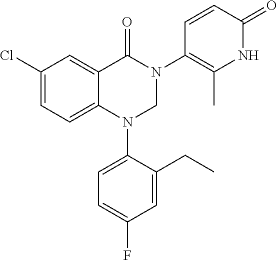 [SMILES: CCc1cc(F)ccc1N1CN(c2ccc(=O)[nH]c2C)C(=O)c2cc(Cl)ccc21] | ¹H NMR (400 MHz, DMSO-d₆) δ: 11.78 (br s, 1H), 7.78 (d, J = 2.6 Hz, 1H), 7.41-7.27 (m, 4H), 7.24-7.13 (m, 1H), 6.29- 6.15 (m, 2H), 5.45 (d, J = 9.4 Hz, 0.6H), 5.17 (d, J = 9.8 Hz, 0.4H), 4.94 (d, J = 9.9 Hz, 0.4H), 4.67 (d, J = 9.3 Hz, 0.6H), 2.61-2.55 (m, 2H), 2.16-2.03 (m, 3H), 1.14 (q, J = 7.5 Hz, 3H). MS (m/z) 412.0 (M + H)⁺ | 6-chloro-1-(2- ethyl-4- fluorophenyl)- 3-(6-methoxy- 2- methylpyridin- 3-yl)-2,3- dihydroquinazolin- 4(1H)-one |
| 82 | 6-chloro-3-(2,4- dimethyl-6-oxo- 1,6-dihydropyridin- 3-yl)-1-(4-fluoro-2- isopropylphenyl)- 2,3- dihydroquinazolin- 4(1H)-one | 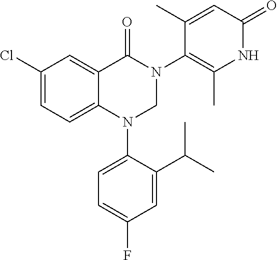 [SMILES: Cc1cc(=O)[nH]c(C)c1N1CN(c2ccc(F)cc2C(C)C)c2ccc(Cl)cc2C1=O] | ¹H NMR (400 MHz, DMSO-d₆) δ: 11.62 (br s, 1H), 7.77 (d, J = 2.3 Hz, 1H), 7.44-7.30 (m, 3H), 7.18 (td, J = 8.3, 2.9 Hz, 1H), 6.16 (d, J = 8.8 Hz, 1H), 6.09 (d, J = 6.1 Hz, 1H), 5.40 (d, J = 9.8 Hz, 1H), 4.69 (dd, J = 12.1, 9.9 Hz, 1H), 3.24-3.12 (m, 1H), 2.12 (d, J = 9.1 Hz, 3H), 2.05 (s, 3H), 1.21-1.05 (m, 6H). MS (m/z) 440.0 (M + H)⁺ | 6-chloro-1-(4- fluoro-2- isopropylphenyl)- 3-(6- methoxy-2,4- dimethylpyridin- 3-yl)-2,3- dihydroquinazolin- 4(1H)-one |
| 83 | 3-(2-ethyl-6-oxo- 1,6-dihydropyridin- 3-yl)-1-(4-fluoro-2- isopropylphenyl)- 7-(trifluoromethyl)- 2,3- dihydroquinazolin- 4(1H)-one | 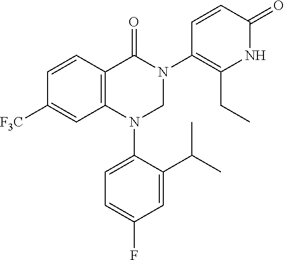 [SMILES: CCc1[nH]c(=O)ccc1N1CN(c2ccc(F)cc2C(C)C)c2cc(C(F)(F)F)ccc2C1=O] | ¹H NMR (400 MHz, DMSO-d₆, as a mixture of rotamers) δ: 11.77 (s, 1H), 8.06 (d, J = 8.00 Hz, 1H), 7.46-7.27 (m, 3H), 7.27-7.16 (m, 2H), 6.36- 6.16 (m, 2H), 5.62 (d, J = 9.60 Hz, 0.6H), 5.17 (d, J = 10.00 Hz, 0.4H), 5.08 (d, J = 10.00 Hz, 0.4H), 4.67 (d, J = 9.20 Hz, 0.6H), 3.23-3.05 (m, 1H), 2.45-2.34 (m, 2H), 1.20- 1.08 (m, 9H). MS (m/z) 474.0 (M + H)⁺ | 3-(2-ethyl-6- methoxypyridin- 3-yl)-1-(4- fluoro-2- isopropylphenyl)- 7- (trifluoromethyl)- 2,3- dihydroquinazolin- 4(1H)-one |
| 84 | 1-(4-fluoro-2- methylphenyl)-3- (5-oxo-4,5- dihydropyrazin-2- yl)-7- (trifluoromethyl)- 2,3- dihydroquinazolin- 4(1H)-one | 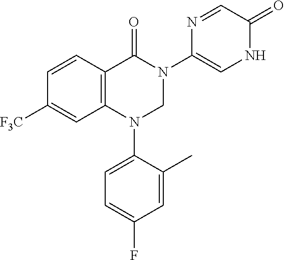 [SMILES: Cc1cc(F)ccc1N1CN(c2c[nH]c(=O)cn2)C(=O)c2ccc(C(F)(F)F)cc21] | ¹H NMR (400 MHz, DMSO-d₆) δ: 12.11 (br s, 1H), 8.41-8.20 (br s 1H), 8.15 (d, J = 8.00 Hz, 1H), 7.94 (d, J = 1.6 Hz, 1H), 7.46 (dd, J = 8.80 Hz, 5.20, 1H), 7.34-7.20 (m, 3H), 6.42 (s, 1H), 5.61- 5.29 (m, 2H), 2.194 (s, 3H). MS (m/z) 419.0 (M + H)⁺ | 1-(4-fluoro-2- methylphenyl)- 3-(5- methoxypyrazin- 2-yl)-7- (trifluoromethyl)- 2,3- dihydroquinazolin- 4(1H)-one |
| 85 | 6-fluoro-1-(4- fluoro-2- methylphenyl)-7- methyl-3-(2- methyl-6-oxo-1,6- dihydropyridin-3- yl)-2,3- dihydroquinazolin- 4(1H)-one | 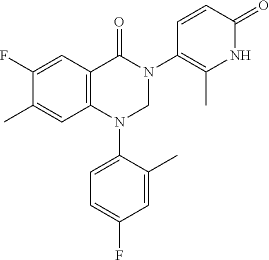 [SMILES: Cc1cc2c(cc1F)C(=O)N(c1ccc(=O)[nH]c1C)CN2c1ccc(F)cc1C] | ¹H NMR (400 MHz, DMSO-d₆) δ: 11.74 (s, 1H), 7.51 (d, J = 9.60 Hz, 1H), 7.33-7.09 (m, 4H), 6.28-6.12 (m, 2H), 5.45- 4.62 (m, 2H), 2.24 (s, 3H), 2.15 (s, 3H), 2.05 (s, 3H). MS (m/z) 396.1 (M + H)⁺ | 6-fluoro-1-(4- fluoro-2- methylphenyl)- 3-(6-methoxy- 2- methylpyridin- 3-yl)-7-methyl- 2,3- dihydroquinazolin- 4(1H)-one |
| 86 | 3-(2-methyl-6-oxo- 1,6-dihydropyridin- 3-yl)-1-phenyl-7- (trifluoromethyl)- 2,3- dihydroquinazolin- 4(1H)-one | 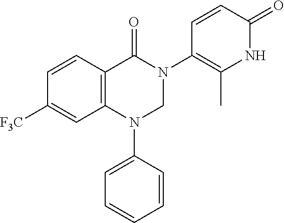 [SMILES: Cc1[nH]c(=O)ccc1N1CN(c2ccccc2)c2cc(C(F)(F)F)ccc2C1=O] | ¹H NMR (400 MHz, DMSO-d₆) δ: 11.76 (s, 1H), 8.11 (d, J = 8.40 Hz, 1H), 7.49-7.44 (m, 2H), 7.38 (dd, J = 8.20, 0.80 Hz, 1H), 7.34-7.24 (m, 4H), 7.10 (s, 1H), 6.19 (d, J = 10.00 Hz, 1H), 5.38 (d, J = 10.80 Hz, 1H), 5.16 (d, J = 10.80 Hz, 1H), 1.95 (s, 3H). MS (m/z) 400.1 (M + H)⁺ | 3-(6-methoxy- 2- methylpyridin- 3-yl)-1-phenyl- 7- (trifluoromethyl)- 2,3- dihydroquinazolin- 4(1H)-one |
| 87 | 3-(2-methyl-6-oxo- 1,6-dihydropyridin- 3-yl)-1-(o-tolyl)-7- (trifluoromethyl)- 2,3- dihydroquinazolin- 4(1H)-one | 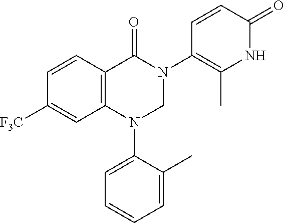 [SMILES: Cc1ccccc1N1CN(c2ccc(=O)[nH]c2C)C(=O)c2ccc(C(F)(F)F)cc21] | ¹H NMR (400 MHz, DMSO-d₆) δ: 11.78 (s, 1H), 8.07 (d, J = 8.00 Hz, 1H), 7.46-7.42 (m, 1H), 7.49-7.32 (m, 4H), 7.25- 7.23 (m, 1H), 6.35 (br s, 1H), 6.20 (d, J = 9.60 Hz, 1H), 5.51-4.80 (m 2H), 2.22 (s, 3H), 2.10 (s, 3H). MS (m/z) 414.1 (M + H)⁺ | 3-(6-methoxy- 2- methylpyridin- 3-yl)-1-(o- tolyl)-7- (trifluoromethyl)- 2,3- dihydroquinazolin- 4(1H)-one |
| 88 | 5-chloro-1-(4- fluoro-2- methylphenyl)-3- (2-methyl-6-oxo- 1,6-dihydropyridin- 3-yl)-2,3- dihydroquinazolin- 4(1H)-one | 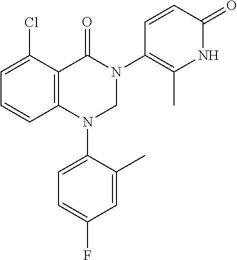 [SMILES: Cc1cc(F)ccc1N1CN(c2ccc(=O)[nH]c2C)C(=O)c2c(Cl)cccc21] | ¹H NMR (400 MHz, DMSO-d₆) δ: 11.74 (s, 1H), 7.31-7.24 (m, 4H), 7.19-7.12 (m, 1H), 7.00 (dd, J = 8.00, 0.80 Hz, 1H), 6.25 (brs, 1H), 6.17 (d, J = 9.60 Hz, 1H), 5.40-4.78 (m, 2H), 2.21 (s, 3H), 2.08 (s, 3H). MS (m/z) 398.0 (M + H)⁺ | 5-chloro-1-(4 - fluoro-2- methylphenyl)- 3-(6-methoxy- 2- methylpyridin- 3-yl)-2,3- dihydroquinazolin- 4(1H)-one |
| 89 | 1-(4-fluoro-2- methylphenyl)-5- methyl-3-(2- methyl-6-oxo-1,6- dihydropyridin-3- yl)-2,3- dihydroquinazolin- 4(1H)-one | 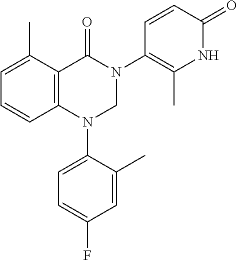 [SMILES: Cc1cc(F)ccc1N1CN(c2ccc(=O)[nH]c2C)C(=O)c2c(C)cccc21] | ¹H NMR (400 MHz, DMSO-d₆, 80° C.) δ: 11.40 (br s, 1H), 7.27- 7.15 (m, 4H), 7.12-7.03 (m, 1H), 6.76 (d, J = 7.20 Hz, 1H), 6.17-6.11 (m, 2H), 5.00 (brs, 2H), 2.63 (s, 3H), 2.23 (s, 3H), 2.10 (s, 3H). MS (m/z) 378.1 (M + H)⁺ | 1-(4-fluoro-2- methylphenyl)- 3-(6-methoxy- 2- methylpyridin- 3-yl)-5-methyl- 2,3- dihydroquinazolin- 4(1H)-one |
| 90 | 3-(2-cyclopropyl-6- oxo-1,6- dihydropyridin-3- yl)-1-(4-fluoro-2- methylphenyl)-7- (trifluoromethyl)- 2,3- dihydroquinazolin- 4(1H)-one | 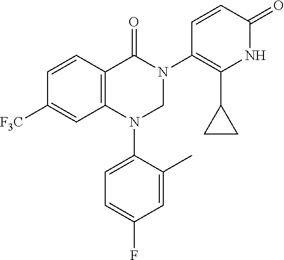 [SMILES: Cc1cc(F)ccc1N1CN(c2ccc(=O)[nH]c2C2CC2)C(=O)c2ccc(C(F)(F)F)cc21] | ¹H NMR (400 MHz, DMSO-d₆, as a mixture of rotamers) δ: 10.88 (s, 1H), 8.09 (d, J = −8.00 Hz, 1H), 7.46-7.20 (m, 5H), 6.47-6.28 (m, 2H), 5.58- 4.77 (m, 2H), 2.22 (s, 3H), 2.01 (m, 1H), 1.07 (d, J = 5.60 Hz, 1H), 0.85-0.75 (m, 3H). MS (m/z) 458.1 (M + H)⁺ | 3-(2- cyclopropyl-6- methoxypyridin- 3-yl)-1-(4- fluoro-2- methylphenyl)- 7- (trifluoromethyl)- 2,3- dihydroquinazolin- 4(1H)-one |
| 91 | 6-chloro-1-(4- fluoro-2- methoxyphenyl)-3- (2-methyl-6-oxo- 1,6-dihydropyridin- 3-yl)-2,3- dihydroquinazolin- 4(1H)-one | 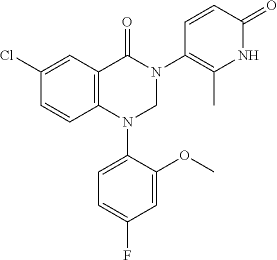 [SMILES: COc1cc(F)ccc1N1CN(c2ccc(=O)[nH]c2C)C(=O)c2cc(Cl)ccc21] | ¹H NMR (400 MHz, DMSO-d₆) δ: 11.76 (s, 1H), 7.76 (d, J = 2.80 Hz, 1H), 7.39-7.27 (m, 3H), 7.14-7.10 (m, 1H), 6.89- 6.83 (m, 1H), 6.39 (d, J = 8.80 Hz, 1H), 6.18 (d, J = 9.60 Hz, 1H), 5.20 (d, J = 10.40 Hz, 1H), 4.81 (d, J = 10.40 Hz, 1H), 3.78 (s, 3H), 2.06 (s, 3H). MS (m/z) 413.8 (M)⁺ | 6-chloro-1-(4- fluoro-2- methoxyphenyl)- 3-(6- methoxy-2- methylpyridin- 3-yl)-2,3- dihydroquinazolin- 4(1H)-one |
| 92 | 1-(4-fluoro-2- methylphenyl)-3- (2-methyl-5-oxo- 2,5-dihydro-1H- pyrazol-3-yl)-7- (trifluoromethyl)- 2,3- dihydroquinazolin- 4(1H)-one | 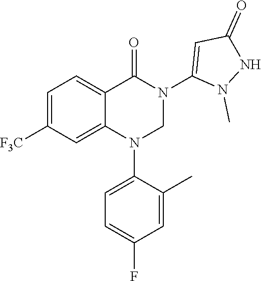 [SMILES: Cc1cc(F)ccc1N1CN(c2cc(=O)[nH]n2C)C(=O)c2ccc(C(F)(F)F)cc21] | ¹H NMR (400 MHz, DMSO-d₆): δ 9.79 (br s, 1H), 8.11 (d, J = 8.0 Hz, 1H), 7.40 (dd, J = 8.8, 5.3 Hz, 1H), 7.33 (dd, J = 9.5, 3.0 Hz, 1H), 7.27 (d, J = 8.5 Hz, 1H), 7.19 (td, J = 8.5, 3.0 Hz, 1H), 6.44 (s, 1H), 5.49 (s, 1H), 5.36 (br s, 1H), 5.02 (br s, 1H), 3.44 (s, 3H), 2.24 (s, 3H). MS (m/z) 421.2 (M + H)⁺ | 1-(4-fluoro-2- methylphenyl)- 3-(3-methoxy- 1-methyl-1H- pyrazol-5-yl)-7- (trifluoromethyl)- 2,3- dihydroquinazolin- 4(1H)-one |
| 93 | 7-fluoro-1-(4- fluoro-2- isopropylphenyl)- 3-(2-methyl-6-oxo- 1,6-dihydropyridin- 3-yl)-2,3- dihydroquinazolin- 4(1H)-one | 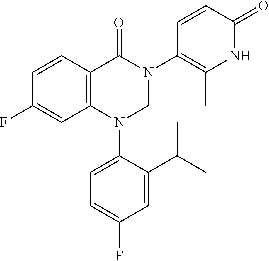 [SMILES: Cc1[nH]c(=O)ccc1N1CN(c2ccc(F)cc2C(C)C)c2cc(F)ccc2C1=O] | ¹HNMR (400 MHz, DMSO-d₆): δ 11.78 (br s, 1H), 7.90 (dd, J = 8.6, 6.6 Hz, 1H), 7.43-7.25 (m, 3H), 7.24-7.16 (m, 1H), 6.72 (td, J = 8.6, 2.0 Hz, 1H), 6.19 (d, J = 9.5 Hz, 1H), 5.90-5.75 (m, 1H), 5.49 (d, J = 9.4 Hz, 0.6H), 5.22 (d, J = 10.0 Hz, 0.4H), 4.88 (d, J = 10.0 Hz, 0.4H), 4.61 (d, J = 9.4 Hz, 0.6H), 3.26- 3.07 (m, 1H), 2.17-2.07 (m, 3H), 1.21-1.05 (m, 6H). | 7-fluoro-1-(4- fluoro-2- isopropylphenyl)- 3-(6-methoxy-2- methylpyridin- 3-yl)-2,3- dihydroquinazolin- 4(1H)-one |
| | | | MS (m/z) 410.0 (M + H)⁺ | |
| 94 | 3-(4,6-dimethyl-2- oxo-1,2- dihydropyrimidin- 5-yl)-1-(4-fluoro-2- methylphenyl)-7- (trifluoromethyl)- 2,3- dihydroquinazolin- 4(1H)-one | 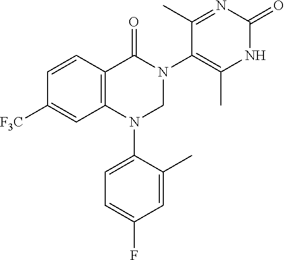 [SMILES: Cc1cc(F)ccc1N1CN(c2c(C)nc(=O)[nH]c2C)C(=O)c2ccc(C(F)(F)F)cc21] | ¹HNMR (400 MHz, DMSO-d₆): δ 12.00 (br s, 1H), 8.08 (d, J = 8.0 Hz, 1H), 7.41 (dd, J = 8.7, 5.4 Hz, 1H), 7.34 (dd, J = 9.7, 2.8 Hz, 1H), 7.26 (dd, J = 8.2, 1.1 Hz, 1H), 7.24-7.18 (m, 1H), 6.36 (d, J = 1.0 Hz, 1H), 5.45 (d, J = 10.0 Hz, 1H), 4.95 (d, J = 10.0 Hz, 1H), 2.23 (s, 3H), 2.20-2.15 (m, 6H). MS (m/z) 447.0 (M + H)⁺ | 1-(4-fluoro-2- methylphenyl)- 3-(2-methoxy- 4,6- dimethylpyrimidin- 5-yl)-7- (trifluoromethyl)- 2,3- dihydroquinazolin- 4(1H)-one |
| 95 | 1-(2-(tert-butyl)-4- fluorophenyl)-6- chloro-3-(2- methyl-6-oxo-1,6- dihydropyridin-3- yl)-2,3- dihydroquinazolin- 4(1H)-one | 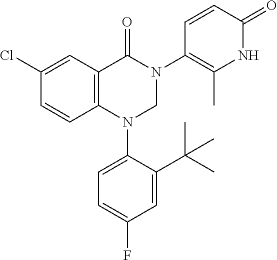 [SMILES: Cc1[nH]c(=O)ccc1N1CN(c2ccc(F)cc2C(C)(C)C)c2ccc(Cl)cc2C1=O] | ¹HNMR (400 MHz, DMSO-d₆): δ 11.81 (br s, 1H), 7.81-7.76 (m, 1H), 7.45-7.30 (m, 3H), 7.29- 7.22 (m, 1H), 7.18 (d, J = 9.5 Hz, 1H), 6.20 (dd, J = 9.5, 3.8 Hz, 1H), 6.06 (dd, J = 8.9, 4.1 Hz, 1H), 5.61 (d, J = 9.0 Hz, 0.6H), 5.49 (d, J = 9.6 Hz, 0.4H), 4.47 (d, J = 9.6 Hz, 0.4H), 4.43 (d, J = 9.0 Hz, 0.6 H), 2.16- 2.03 (m, 3H), 1.39-1.31 (m, 9H). MS (m/z) 440.2 (M + H)⁺ | 1-(2-(tert- butyl)-4- fluorophenyl)- 6-chloro-3-(6- methoxy-2- methylpyridin- dihydroquinazolin- 4(1H)-one |
| 96 | 1-(3,4-difluoro-2- methylphenyl)-3- (2-methyl-6-oxo- 1,6-dihydropyridin- 3-yl)-6- (trifluoromethyl)- 2,3- dihydroquinazolin- 4(1H)-one | 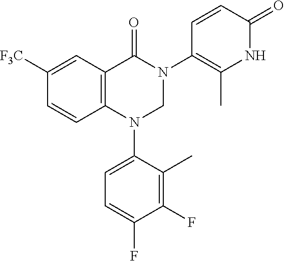 [SMILES: Cc1[nH]c(=O)ccc1N1CN(c2ccc(F)c(F)c2C)c2ccc(C(F)(F)F)cc2C1=O] | ¹HNMR (400 MHz, DMSO-d₆): δ 11.80 (br s, 1H), 8.09 (d, J = 2.0 Hz, 1H), 7.65 (dd, J = 8.8, 2.1 Hz, 1H), 7.51-7.36 (m, 2H), 7.29-7.22 (m, 1H), 6.53-6.37 (m, 1H), 6.20 (d, J = 9.5 Hz, 1H), 5.51 (br d, J = 9.5 Hz, 0.6H), 5.22 (br d, J = 9.4 Hz, 0.4H), 5.11 (br d, J = 10.0 Hz, 0.4H), 4.82 (br d, J = 9.3 Hz, 0.6H), 2.18 (br s, 3H), 2.14-2.06 (m, 3H). MS (m/z) 450.0 (M + H)⁺ | 1-(3,4-difluoro- 2- methylphenyl)- 3-(6-methoxy- 2- methylpyridin- 3-yl)-6- (trifluoromethyl)- 2,3- dihydroquinazolin- 4(1H)-one |
| 97 | 6-chloro-1-(4- fluoro-2- isopropylphenyl)- 5-methyl-3-(2- methyl-6-oxo-1,6- dihydropyridin-3- yl)-2,3- dihydroquinazolin- 4(1H)-one | 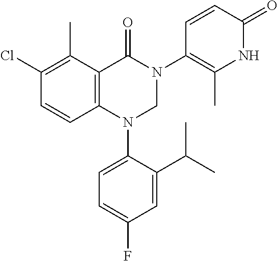 [SMILES: Cc1[nH]c(=O)ccc1N1CN(c2ccc(F)cc2C(C)C)c2ccc(Cl)c(C)c2C1=O] | ¹HNMR (400 MHz, DMSO-d₆): δ 11.76 (br s, 1H), 7.40-7.22 (m, 4H), 7.19-7.11 (m, 1H), 6.18 (d, J = 9.5 Hz, 1H), 6.09 (dd, J = 13.5, 8.9 Hz, 1H), 5.26 (d, J = 10.1 Hz, 0.6H), 5.12 (d, J = 10.6 Hz, 0.4H), 4.81 (d, J = 10.6 Hz, 0.4H), 4.68 (d, J = 10.1 Hz, 0.6H), 3.21- 3.04 (m, 1H), 2.64 (d, J = 5.0 Hz, 3H), 2.10 (d, J = 6.6 Hz, 3H), 1.19-1.09 (m, 6H) MS (m/z) 440.0 (M + H)⁺ | 6-chloro-1-(4- fluoro-2- isopropylphenyl)- 3-(6- methoxy-2- methylpyridin- 3-yl)-5-methyl- 2,3- dihydroquinazolin- 4(1H)-one |
| 98 | 6-chloro-7-fluoro- 1-(4-fluoro-2- isopropylphenyl)- 3-(2-methyl-6-oxo- 1,6-dihydropyridin- 3-yl)-2,3- dihydroquinazolin- 4(1H)-one | 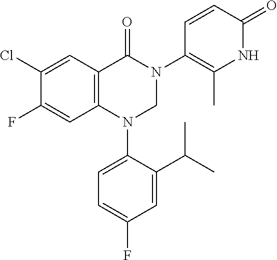 [SMILES: Cc1[nH]c(=O)ccc1N1CN(c2ccc(F)cc2C(C)C)c2cc(F)c(Cl)cc2C1=O] | ¹HNMR (400 MHz, DMSO-d₆): δ 11.80 (s, 1H), 7.92 (d, J = 8.40 Hz, 1H), 7.30-7.28 (m, 3H), 7.19-7.17 (m, 1H), 6.20 (d, J = 9.60 Hz, 1H), 6.15-6.02 (m, 1H), 5.49 (d, J = 9.20 Hz, 0.5H), 5.22 (d, J = 10.00 Hz, 0.5H), 4.93 (d, J = 10.40 Hz, 0.5H), 4.66 (d, J = 9.60 Hz, 0.5H), 3.09-3.01 (m, 1H), 2.12-2.09 (m, 3H), 1.15-1.13 (m, 6H). MS (m/z) 444.0 (M + H)⁺ | 6-chloro-7- fluoro-1-(4- fluoro-2- isopropylphenyl)- 3-(6- methoxy-2- methylpyridin- 3-yl)-2,3- dihydroquinazolin- 4(1H)-one |
| 99 | 1-(4-fluoro-2- methylphenyl)-7- methoxy-3-(2- methyl-6-oxo-1,6- dihydropyridin-3- yl)-2,3- dihydroquinazolin- 4(1H)-one | 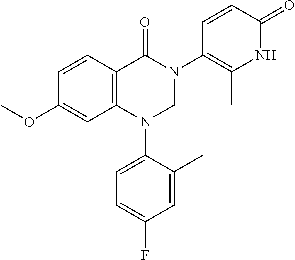 [SMILES: COc1ccc2c(c1)N(c1ccc(F)cc1C)CN(c1ccc(=O)[nH]c1C)C2=O] | ¹HNMR (400 MHz, DMSO-d₆): δ 11.38 (s, 1H), 7.82 (d, J = 8.80 Hz, 1H), 7.28-7.28 (m, 2H), 7.23 (dd, J = 2.80, 9.60 Hz, 1H), 7.11 (dt, J = 2.80, 12.27 Hz, 1H), 6.53 (dd, J = 2.40, 8.60 Hz, 1H), 6.17 (d, J = 9.20 Hz, 1H), 5.65 (d, J = 2.00 Hz, 1H), 5.25-4.65 (br s, 2H), 3.66 (s, 3H), 2.26 (s, 3H), 2.11 (s, 3H). MS (m/z) 394.2 (M + H)⁺ | 1-(4-fluoro-2- methylphenyl)- 7-methoxy-3- (6-methoxy-2- methylpyridin- 3-yl)-2,3- dihydroquinazolin- 4(1H)-one |
| 100 | 1-(4-fluoro-2- methylphenyl)-6- hydroxy-3-(2- methyl-6-oxo-1,6- dihydropyridin-3- yl)-7- (trifluoromethyl)- 2,3- dihydroquinazolin- 4(1H)-one | 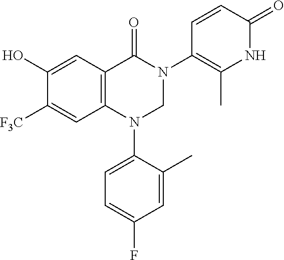 [SMILES: Cc1cc(F)ccc1N1CN(c2ccc(=O)[nH]c2C)C(=O)c2cc(O)c(C(F)(F)F)cc21] | ¹HNMR (400 MHz, DMSO-d₆: δ 11.75 (s, 1H), 10.43 (s, 1H), 7.57 (s, 1H), 7.30-7.24 (m, 2H), 7.19-7.06 (m, 2H), 6.46 (br s, 1H), 6.17 (d, J = 9.20 Hz, 1H), 5.19 (d, J = 10.00 Hz, 1H), 4.82 (d, J = 10.40 Hz, 1H), 2.26 (s, 3H), 2.01 (br s, 3H). MS (m/z) 448.0 (M + H)⁺ | |
| 101 | 1-(4-fluoro-2- methylphenyl)-6- methoxy-3-(2- methyl-6-oxo-1,6- dihydropyridin-3- yl)-7- (trifluoromethyl)- 2,3- dihydroquinazolin- 4(1H)-one | 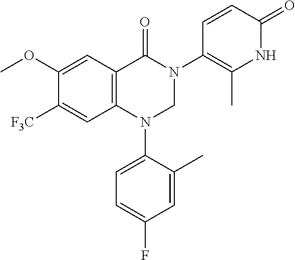 [SMILES: COc1cc2c(cc1C(F)(F)F)N(c1ccc(F)cc1C)CN(c1ccc(=O)[nH]c1C)C2=O] | ¹HNMR (400 MHz, DMSO-d₆): δ 11.77 (s, 1H), 7.65 (s, 1H), 7.32- 7.13 (m, 3H), 7.12-7.07 (m, 1H), 6.53 (br s, 1H), 6.19 (d, J = 9.60 Hz, 1H), 5.28-5.39 (br s, 1H), 4.83-4.95 (br s, 1H), 3.91 (s, 3H), 2.26 (s, 3H), 2.04 (br s, 3H). MS (m/z) 462.2 (M + H)⁺ | 1-(4-fluoro-2- methylphenyl)- 6-methoxy-3- (6-methoxy-2- methylpyridin- 3-yl)-7- (trifluoromethyl)- 2,3- dihydroquinazolin- 4(1H)-one |
| 102 | 6-chloro-1-(4- fluoro-2- methylphenyl)-3- (2-methyl-6-oxo- 1,6-dihydropyridin- 3-yl)-2,3- dihydropyrido[2,3- d]pyrimidin-4(1H)- one | 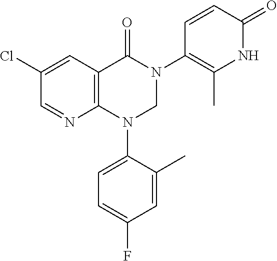 [SMILES: Cc1cc(F)ccc1N1CN(c2ccc(=O)[nH]c2C)C(=O)c2cc(Cl)cnc21] | ¹HNMR (400 MHz, DMSO-d₆): δ 11.50 (s, 1H), 8.24 (d, J = 2.80 Hz, 1H), 8.11 (d, J = 2.80 Hz, 1H), 7.37-7.29 (m, 2H), 7.18 (dd, J = 9.60, 3.20, Hz, 1H), 7.11-7.05 (m, 1H), 6.21 (d, J = 9.60 Hz, 1H), 5.43-4.91 (m, 2H), 2.21 (s, 3H), 2.15 (s, 3H). MS (m/z) 399.0 (M + H)⁺ | 6-chloro-1-(4- fluoro-2- methylphenyl)- 3-(6-methoxy-2- methylpyridin- 3-yl)-2,3- dihydropyrido[2,3- d]pyrimidin- 4(1H)-one |
| 103 | 1-(4-fluoro-2- isopropylphenyl)- 3-(2-methyl-6-oxo- 1,6-dihydropyridin- 3-yl)-7- (trifluoromethoxy)- 2,3- dihydroquinazolin- 4(1H)-one | 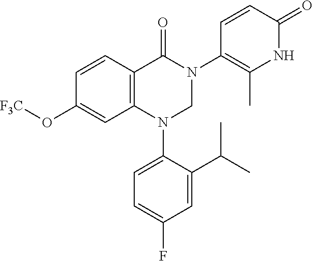 [SMILES: Cc1[nH]c(=O)ccc1N1CN(c2ccc(F)cc2C(C)C)c2cc(OC(F)(F)F)ccc2C1=O] | ¹HNMR (400 MHz, DMSO-d₆): δ 11.80 (s, 1H), 7.97 (d, J = 8.80 Hz, 1H), 7.24-7.23 (m, 3H), 7.20-7.19 (m, 1H), 6.85 (dd, J = 0.80, 8.40 Hz, 1H), 6.20 (d, J = 9.20 Hz, 1H), 5.94-5.89 (m, 1H), 5.54 (d, J = 9.20 Hz, 0.6H), 5.26 (d, J = 10.00 Hz, 0.4H), 4.93 (d, J = 10.00 Hz, 0.4H), 4.66 (d, J = 9.20 Hz, 0.6H), 3.09- 3.07 (m, 1H), 2.13 (s, 3H), 1.12-1.10 (m, 6H). MS (m/z) 476.0 (M + H)⁺ | 1-(4-fluoro-2- isopropylphenyl)- 3-(6- methoxy-2- methylpyridin- 3-yl)-7- (trifluorometh- oxy)-2,3- dihydroquinazolin- 4(1H)-one |
| 104 | 6-chloro-7-fluoro- 1-(4-fluoro-2- methylphenyl)-3- (2-methyl-6-oxo- 1,6-dihydropyridin- 3-yl)-2,3- dihydroquinazolin- 4(1H)-one | 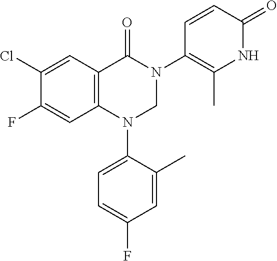 [SMILES: Cc1cc(F)ccc1N1CN(c2ccc(=O)[nH]c2C)C(=O)c2cc(Cl)c(F)cc21] | ¹HNMR (400 MHz, DMSO-d₆): δ 11.78 (br s, 1H), 7.94 (d, J = 8.40 Hz, 1H), 7.34-7.32 (m, 2H), 7.26 (dd, J = 2.80, 9.60 Hz, 1H), 7.15 (td, J = 3.20, 8.40 Hz, 1H), 6.19 (d, J = 9.60 Hz, 1H), 6.12 (d, J = 11.20 Hz, 1H), 5.39-4.85 (br s, 2H), 2.26 (s, 3H), 2.13 (s, 3H). MS (m/z) 416.0 (M + H)⁺ | 6-chloro-7- fluoro-1-(4- fluoro-2- methylphenyl)- 3-(6-methoxy-2- methylpyridin- 3-yl)-2,3- dihydroquinazolin- 4(1H)-one |
| 105 | 6-chloro-1-(4- fluoro-2- methylphenyl)-3- (2-methyl-6-oxo- 1,6-dihydropyridin- 3-yl)-4-oxo- 1,2,3,4- tetrahydroquinazoline- 7-carbonitrile | 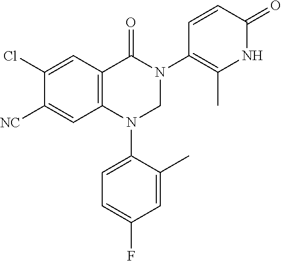 [SMILES: Cc1cc(F)ccc1N1CN(c2ccc(=O)[nH]c2C)C(=O)c2cc(Cl)c(C#N)cc21] | ¹HNMR (400 MHz, DMSO-d₆): δ 11.80 (s, 1H), 8.00 (s, 1H), 7.41- 7.30 (m, 3H), 7.18 (s, 1H), 6.82-6.72 (m, 1H), 6.20 (d, J = 9.60 Hz, 1H), 5.44-4.80 (m, 2H), 2.23 (s, 3H), 2.10 (s, 3H). MS (m/z) 423.0 (M + H)⁺ | 6-chloro-1-(4- fluoro-2- methylphenyl)- 3-(6-methoxy- 2- methylpyridin- 3-yl)-4-oxo- 1,2,3,4- tetrahydroquin- azoline-7- carbonitrile |
| 106 | 6-chloro-3-(2- ethyl-6-oxo-1,6- dihydropyridin-3- yl)-1-(4-fluoro-2- methylphenyl)-2,3- dihydroquinazolin- 4(1H)-one | 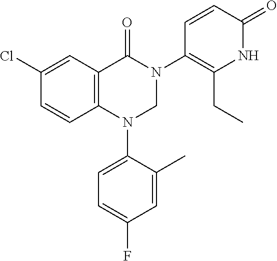 [SMILES: CCc1[nH]c(=O)ccc1N1CN(c2ccc(F)cc2C)c2ccc(Cl)cc2C1=O] | ¹HNMR (400 MHz, DMSO-d₆): δ 11.74 (s, 1H), 7.79 (d, J = 2.40 Hz, 1H), 7.41-7.36 (m, 2H), 7.30-7.28 (m, 2H), 7.20- 2.11 (m, 1H), 6.33-6.20 (m, 2H), 5.48-5.11 (m, 1H), 4.99-4.66 (m, 1H), 2.41-2.32 (m, 2H), 2.21 (s, 3H), 1.04 (s, 3H). MS (m/z) 412.0 (M + H)⁺ | 6-chloro-3-(2- ethyl-6- methoxypyridin- 3-yl)-1-(4- fluoro-2- methylphenyl)-2,3- dihydroquinazolin- 4(1H)-one |
| 107 | 7-fluoro-1-(4- fluoro-2- methylphenyl)-3- (2-methyl-6-oxo- 1,6-dihydropyridin- 3-yl)-6- (trifluoromethyl)- 2,3- dihydroquinazolin- 4(1H)-one | 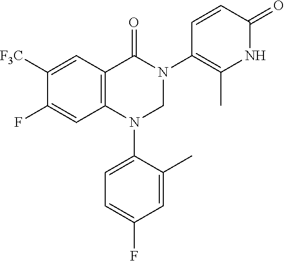 [SMILES: Cc1cc(F)ccc1N1CN(c2ccc(=O)[nH]c2C)C(=O)c2cc(C(F)(F)F)c(F)cc21] | ¹HNMR (400 MHz, DMSO-d₆): δ 11.79 (s, 1H), 8.09 (d, J = 8.40 Hz, 1H), 7.46-7.32 (m, 3H), 7.24-7.20 (m, 1H), 6.21- 6.08 (m, 2H), 5.52 (d, J = 9.20 Hz, 0.6H), 5.23 (d, J = 10.00 Hz, 0.4H), 5.10 (d, J = 10.00 Hz, 0.4H), 4.81 (d, J = 9.60 Hz, 0.6H), 2.25 (s, 3H), 2.12 (s, 3H). MS (m/z) 450.0 (M + H)⁺ | 7-fluoro-1-(4- fluoro-2- methylphenyl)- 3-(6-methoxy-2- methylpyridin- 3-yl)-6- (trifluoromethyl)- 2,3- dihydroquinazolin- 4(1H)-one |
| 108 | 5-fluoro-1-(4- fluoro-2- methylphenyl)-3- (2-methyl-6-oxo- 1,6-dihydropyridin- 3-yl)-6- (trifluoromethyl)- 2,3- dihydroquinazolin- 4(1H)-one | 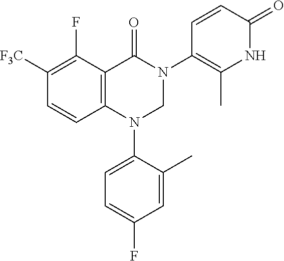 [SMILES: Cc1cc(F)ccc1N1CN(c2ccc(=O)[nH]c2C)C(=O)c2c1ccc(C(F)(F)F)c2F] | ¹HNMR (400 MHz, DMSO-d₆): δ 11.78 (s, 1H), 7.62 (t, J = 8.00 Hz, 1H), 7.41-7.31 (m, 3H), 7.22-7.18 (m, 1H), 6.20- 6.10 (m, 2H), 5.44-4.82 (m, 2H), 2.22 (s, 3H), 2.11 (s, 3H). MS (m/z) 450.0 (M + H)⁺ | 5-fluoro-1-(4- fluoro-2- methylphenyl)- 3-(6-methoxy-2- methylpyridin- 3-yl)-6- (trifluoromethyl)- 2,3- dihydroquinazolin- 4(1H)-one |
| 109 | 7-chloro-6-fluoro- 1-(4-fluoro-2- methylphenyl)-3- (2-methyl-6-oxo- 1,6-dihydropyridin- 3-yl)-2,3- dihydroquinazolin- 4(1H)-one | 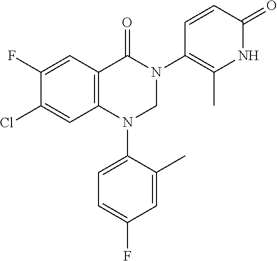 [SMILES: Cc1cc(F)ccc1N1CN(c2ccc(=O)[nH]c2C)C(=O)c2cc(F)c(Cl)cc21] | ¹HNMR (400 MHz, DMSO-d₆): δ 11.76 (s, 1H), 7.76 (d, J = 9.20 Hz, 1H), 7.35-7.28 (m, 3H), 7.20-7.11 (m, 1H), 6.40- 6.31 (m, 1H), 6.18 (d, J = 9.60 Hz, 1H), 5.37-4.77 (m, 2H), 2.25 (s, 3H), 2.08 (s, 3H). MS (m/z) 416.0 (M + H)⁺ | 7-chloro-6- fluoro-1-(4- fluoro-2- methylphenyl)- 3-(6-methoxy-2- methylpyridin- 3-yl)-2,3- dihydroquinazolin- 4(1H)-one |
| 110 | 6-fluoro-1-(4- fluoro-2- methylphenyl)-3- (2-methyl-6-oxo- 1,6-dihydropyridin- 3-yl)-7- (trifluoromethyl)- 2,3- dihydroquinazolin- 4(1H)-one | 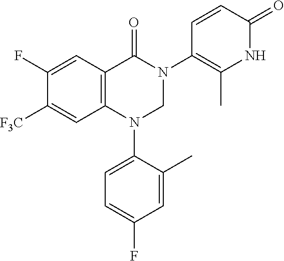 [SMILES: Cc1cc(F)ccc1N1CN(c2ccc(=O)[nH]c2C)C(=O)c2cc(F)c(C(F)(F)F)cc21] | ¹HNMR (400 MHz, DMSO-d₆, at 80° C.): δ 11.46 (s, 1H), 7.84 (d, J = 10.40 Hz, 1H), 7.32- 7.36 (m, 2H), 7.27 (dd, J = 9.60, 2.80 Hz, 1H), 7.12-7.17 (m, 1H), 6.47 (d, J = 5.60 Hz, 1H), 6.20 (d, J = 9.60 Hz, 1H), 4.91-5.39 (m, 2H), 2.27 (s, 3H), 2.12 (s, 3H). MS (m/z) 450.0 (M + H)⁺ | 6-fluoro-1-(4- fluoro-2- methylphenyl)- 3-(6-methoxy- 2- methylpyridin- 3-yl)-7- (trifluoromethyl)- 2,3- dihydroquinazolin- 4(1H)-one |
| 111 | 1-(4-fluoro-2- methylphenyl)-3- (2-methyl-6-oxo- 1,6-dihydropyridin- 3-yl)-4-oxo-6- (trifluoromethyl)- 1,2,3,4- tetrahydroquinazoline- 7-carbonitrile | 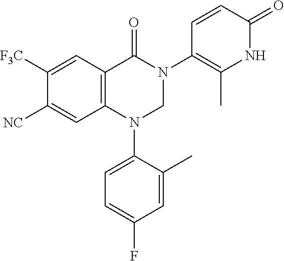 [SMILES: Cc1cc(F)ccc1N1CN(c2ccc(=O)[nH]c2C)C(=O)c2cc(C(F)(F)F)c(C#N)cc21] | ¹HNMR (400 MHz, DMSO-d₆): δ 11.80 (br s, 1H), 8.20 (s, 1H), 7.46- 7.34 (m, 3H), 7.25-7.19 (m, 1H), 6.90-6.83 (m, 1H), 6.21 (d, J = 8.80 Hz, 1H), 5.54-4.88 (m, 2H), 2.25 (s, 3H), 2.12 (s, 3H). MS (m/z) 457.0 (M + H)⁺ | 1-(4-fluoro-2- methylphenyl)- 3-(6-methoxy- 2- methylpyridin- 3-yl)-4-oxo-6- (trifluoromethyl)- 1,2,3,4- tetrahydroquin- azoline-7- carbonitrile |
| 112 | 1-(4-fluoro-2- methylphenyl)-3- (2-methyl-6-oxo- 1,6-dihydropyridin- 3-yl)-4-oxo-7- (trifluoromethoxy)- 1,2,3,4- tetrahydroquinazoline- 6-carbonitrile | 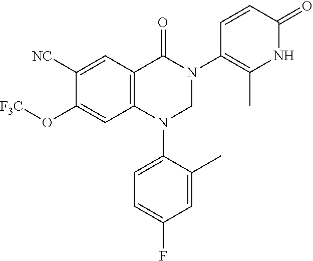 [SMILES: Cc1cc(F)ccc1N1CN(c2ccc(=O)[nH]c2C)C(=O)c2cc(C#N)c(OC(F)(F)F)cc21] | ¹H-NMR (400 MHz, DMSO-d₆, mixture of rotamers): δ 11.82 (s, 1H), 8.33 (s, 1H), 7.50- 7.45 (m, 1H), 7.42-7.35 (m, 2H), 7.28-7.23 (m, 1H), 6.22-6.19 (m, 1H), 6.14-6.11 (m, 1H), 5.57 (d, J = 10.00 Hz, 0.5H), 5.29 (d, J = 10.00 Hz, 0.5H), 5.16 (d, J = 10.00 Hz, 0.5H), 4.90 (d, J = 9.60 Hz, 0.5H), 2.23 (s, 3H), 2.12 (s, 3H). MS (m/z) 472.8 (M)⁺ | 1-(4-fluoro-2- methylphenyl)- 3-(6-methoxy- 2- methylpyridin- 3-yl)-4-oxo-7- (trifluorometh- oxy)-1,2,3,4- tetrahydroquin- azoline-6- carbonitrile |
| 113 | 1-(4,5-difluoro-2- methylphenyl)-3- (2-methyl-6-oxo- 1,6-dihydropyridin- 3-yl)-6- (trifluoromethyl)- 2,3- dihydroquinazolin- 4(1H)-one | 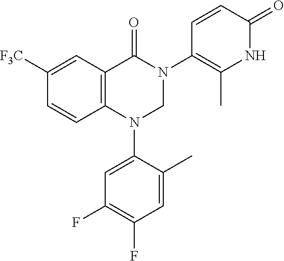 [SMILES: Cc1cc(F)c(F)cc1N1CN(c2ccc(=O)[nH]c2C)C(=O)c2cc(C(F)(F)F)ccc21] | ¹H NMR (400 MHz, DMSO-d₆) δ: 11.81 (br s, 1H), 8.09 (d, J = 2.0 Hz, 1H), 7.67 (dd, J = 8.8, 2.1 Hz, 1H), 7.62-7.54 (m, 2H), 7.45-7.34 (m, 1H), 6.47-6.36 (m, 1H), 6.21 (b rd, J = 9.4 Hz, 1H), 5.49 (br d, J = 8.4 Hz, 0.6H), 5.25 (br d, J = 8.6 Hz, 0.4H), 5.06 (br d, J = 9.1 Hz, 0.4H), 4.82 (br d, J = 9.4 Hz, 0.4H), 2.18 (br s, 3H), 2.11 (s, 3H). MS (m/z) 450.0 (M + H)⁺ | 1-(4,5-difluoro- 2- methylphenyl)- 3-(6-methoxy- 2- methylpyridin- 3-yl)-6- (trifluoromethyl)- 2,3- dihydroquinazolin- 4(1H)-one |
| 114 | 1-(2-(tert-butyl)-4- fluorophenyl)-3-(2- methyl-6-oxo-1,6- dihydropyridin-3- yl)-7- (trifluoromethyl)- 2,3- dihydroquinazolin- 4(1H)-one | 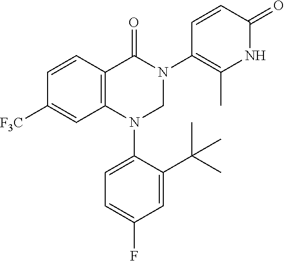 [SMILES: Cc1[nH]c(=O)ccc1N1CN(c2ccc(F)cc2C(C)(C)C)c2cc(C(F)(F)F)ccc2C1=O] | ¹H NMR (400 MHz, DMSO-d₆) δ:11.83 (brs, 1H), 8.06 (dd, J = 7.8, 5.3 Hz, 1H), 7.53-7.34 (m, 3H), 7.33-7.17 (m, 2H), 6.21 (dd, J = 9.4, 5.5 Hz, 1H), 6.17 (br d, J = 2.6 Hz, 1H), 5.71 (d, J = 9.0 Hz, 0.6H), 5.60 (d, J = 9.6 Hz, 0.4H), 4.52 (dd, J = 11.9, 9.3 Hz, 1H), 2.15-2.05 (m, 3H), 1.38-1.33 (m, 9H). MS (m/z) 474.2 (M + H)⁺ | 1-(2-(tert- butyl)-4- fluorophenyl)- 3-(6-methoxy- 2- methylpyridin- 3-yl)-7- (trifluoromethyl)- 2,3- dihydroquinazolin- 4(1H)-one |
| 115 | 1-(4-fluoro-2- methylphenyl)-3- (6-methyl-2-oxo- 1,2- dihydropyrimidin- 5-yl)-7- (trifluoromethyl)- 2,3- dihydroquinazolin- 4(1H)-one | 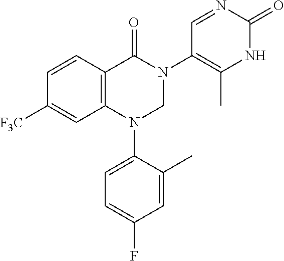 [SMILES: Cc1cc(F)ccc1N1CN(c2cnc(=O)[nH]c2C)C(=O)c2ccc(C(F)(F)F)cc21] | ¹HNMR (400 MHz, DMSO-d₆, 80° C.): δ 8.23- 8.11 (m, 1H), 8.06 (d, J = 8.4 Hz, 1H), 7.32 (dd, J = 8.4, 5.6 Hz, 1H), 7.26- 7.17 (m, 2H), 7.12 (td, J = 8.4, 3.1 Hz, 1H), 6.38 (s, 1H), 5.13 (br s, 2H), 2.21 (s, 3H), 2.18 (s, 3H). MS (m/z) 433.0 (M + H)⁺ | 1-(4-fluoro-2- methylphenyl)- 3-(2-methoxy- 4- methylpyrimidin- 5-yl)-7- (trifluoromethyl)- 2,3- dihydroquinazolin- 4(1H)-one |
| 116 | 1-(2-ethyl-4- fluorophenyl)-6- fluoro-3-(2-methyl- 6-oxo-1,6- dihydropyridin-3- yl)-2,3- dihydroquinazolin- 4(1H)-one | 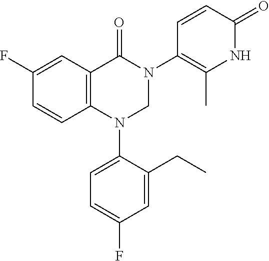 [SMILES: CCc1cc(F)ccc1N1CN(c2ccc(=O)[nH]c2C)C(=O)c2cc(F)ccc21] | ¹HNMR (400 MHz, DMSO-d₆): δ 11.75 (br s, 1H), 7.57 (dd, J = 8.8, 3.1 Hz, 1H), 7.37-7.21 (m, 4H), 7.21-7.10 (m, 1H), 6.37-6.15 (m, 2H), 5.39 (d, J = 7.6 Hz, 0.6H), 5.15-5.04 (m, 0.4H), 5.03-4.92 (m, 0.4H), 4.68 (d, J = 8.5 Hz, 0.6H), 2.64-2.56 (m, 2H), 2.20-1.97 (m, 3H), 1.21-1.09 (m, 3H). MS (m/z) 396.2 (M + H)⁺ | 1-(2-ethyl-4- fluorophenyl)- 6-fluoro-3-(6- methoxy-2- methylpyridin- 3-yl)-2,3- dihydroquinazolin- 4(1H)-one |
| 117 | 4-(6-chloro-3-(2- methyl-6-oxo-1,6- dihydropyridin-3- yl)-4-oxo-3,4- dihydroquinazolin- 1(2H)-yl)-3- methylbenzonitrile | 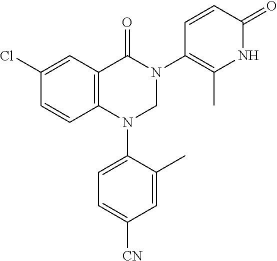 [SMILES: Cc1cc(C#N)ccc1N1CN(c2ccc(=O)[nH]c2C)C(=O)c2cc(Cl)ccc21] | ¹HNMR (400 MHz, DMSO-d₆): δ 11.77 (br s, 1H), 7.90 (d, J = 1.3 Hz, 1H), 7.83 (d, J = 2.5 Hz, 1H), 7.77 (br d, J = 8.4 Hz, 1H), 7.44 (dd, J = 8.8, 2.6 Hz, 1H), 7.41- 7.32 (m, 2H), 6.42 (br d, J = 7.5 Hz, 1H), 6.18 (d, J = 9.5 Hz, 1H), 5.33 (br s, 1H), 4.99 (br s, 1H), 2.22 (s, 3H), 2.02 (s, 3H). MS (m/z) 405.0 (M + H)⁺ | 4-(6-chloro-3- (6-methoxy-2- methylpyridin- 3-yl)-4-oxo- 3,4- dihydroquinazolin- 1(2H)-yl)-3- methylbenzonitrile |
| 118 | 1-(5-fluoro-2- methylphenyl)-3- (2-methyl-6-oxo- 1,6-dihydropyridin- 3-yl)-6- (trifluoromethyl)- 2,3- dihydroquinazolin- 4(1H)-one | 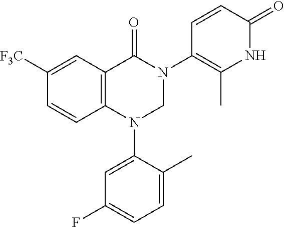 [SMILES: Cc1ccc(F)cc1N1CN(c2ccc(=O)[nH]c2C)C(=O)c2cc(C(F)(F)F)ccc21] | ¹HNMR (400 MHz, DMSO-d₆): δ 11.80 (br s, 1H), 8.09 (d, J = 2.0 Hz, 1H), 7.68 (dd, J = 8.8, 2.1 Hz, 1H), 7.48 (dd, J = 8.3, 6.9 Hz, 1H), 7.42- 7.36 (m, 1H), 7.29 (dd, J = 9.8, 2.6 Hz, 1H), 7.22 (td, J = 8.4, 2.6 Hz, 1H), 6.39 (br s, 1H), 6.20 (d, J = 9.5 Hz, 1H), 5.54 (br s, 0.6H), 5.29 (br s, 0.4H), 5.08 (br s, 0.4H), 4.84 (br s, 0.6H), 2.17 (s, 3H), | 1-(5-fluoro-2- methylphenyl)- 3-(6-methoxy- 2- methylpyridin- 3-yl)-6- (trifluoromethyl)- 2,3- dihydroquinazolin- 4(1H)-one |
| | | | 2.11 (s, 3H). | |
| | | | MS (m/z) 432.2 (M + H)⁺ | |
| 119 | 1-(2-methyl-4- (trifluorometh- oxy)phenyl)-3-(6-oxo- 1,6-dihydropyridin- 3-yl)-6- (trifluoromethyl)- 2,3- dihydropyrido[2,3- d]pyrimidin-4(1H)- one | 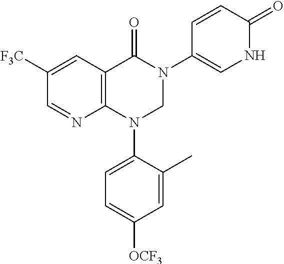 [SMILES: Cc1cc(OC(F)(F)F)ccc1N1CN(c2ccc(=O)[nH]c2)C(=O)c2cc(C(F)(F)F)cnc21] | ¹H NMR (400 MHz, DMSO-d₆) δ: 11.79 (s, 1H), 8.60 (s, 1H), 8.32 (d, J = 2.00 Hz, 1H), 7.57-7.48 (m, 3H), 7.41 (s, 1H), 7.32 (d, J = 8.80 Hz, 1H), 6.38 (d, J = 9.60 Hz, 1H), 5.55 (d, J = 9.60 Hz, 1H), 5.20 (d, J = 9.60 Hz, 1H), 2.23 (s, 3H). MS (m/z) 484.8 (M + H)⁺ | 3-(6- methoxypyridin- 3-yl)-1-(2- methyl-4- (trifluorometh- oxy)phenyl)-6- (trifluoromethyl)- 2,3- dihydropyrido[2,3- d]pyrimidin- 4(1H)-one |
| 120 | 1-(3-fluoro-2- methylphenyl)-3- (2-methyl-6-oxo- 1,6-dihydropyridin- 3-yl)-6- (trifluoromethyl)- 2,3- dihydroquinazolin- 4(1H)-one | 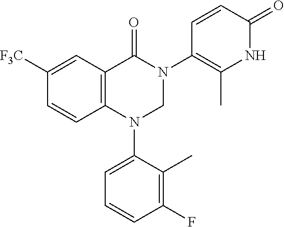 [SMILES: Cc1[nH]c(=O)ccc1N1CN(c2cccc(F)c2C)c2ccc(C(F)(F)F)cc2C1=O] | ¹H NMR (400 MHz, DMSO-d₆) δ: 11.78 (br s, 1H), 8.10 (s, 1H), 7.67 (d, J = 8.9 Hz, 1H), 7.46- 7.35 (m, 2H), 7.33- 7.16 (m, 2H), 6.55-6.35 (m, 1H), 6.30-6.10 (m, 1H), 5.55 (br s, 0.6H), 5.24 (br s, 0.4H), 5.15 (br s, 0.4H), 4.84 (br s, 0.6H), 2.13 (br s, 6H). MS (m/z) 432.0 (M + H)⁺ | 1-(3-fluoro-2- methylphenyl)- 3-(6-methoxy- 2- methylpyridin- 3-yl)-6- (trifluoromethyl)- 2,3- dihydroquinazolin- 4(1H)-one |
| 121 | 1-(2,4-difluoro-6- methylphenyl)-3- (2-methyl-6-oxo- 1,6-dihydropyridin- 3-yl)-6- (trifluoromethyl)- 2,3- dihydroquinazolin- 4(1H)-one | 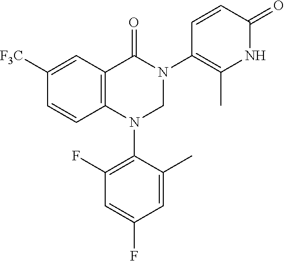 [SMILES: Cc1cc(F)cc(F)c1N1CN(c2ccc(=O)[nH]c2C)C(=O)c2cc(C(F)(F)F)ccc21] | ¹H NMR (400 MHz, DMSO-d₆) δ: 11.83 (br s, 1H), 8.10 (d, J = 1.6 Hz, 1H), 7.68 (dd, J = 8.7, 1.7 Hz, 1H), 7.48-7.31 (m, 2H), 7.28-7.21 (m, 1H), 6.48-6.37 (m, 1H), 6.21 (d, J = 9.5 Hz, 1H), 5.45 (d, J = 9.5 Hz, 0.6H), 5.25 (d, J = 10.5 Hz, 0.4H), 5.08 (d, J = 10.4 Hz, 0.4H), 4.85 (d, J = 9.8 Hz, 0.6H), 2.27 (s, 3H), 2.16-2.07 (m, 3H). MS (m/z) 450.0 (M + H)⁺ | 1-(2,4-difluoro- 6- methylphenyl)- 3-(6-methoxy- 2- methylpyridin- 3-yl)-6- (trifluoromethyl)- 2,3- dihydroquinazolin- 4(1H)-one |
| 122 | 7-(difluoromethyl)- 1-(4-fluoro-2- isopropylphenyl)- 3-(2-methyl-6-oxo- 1,6-dihydropyridin- 3-yl)-2,3- dihydropyrido[2,3- d]pyrimidin-4(1H)- one | 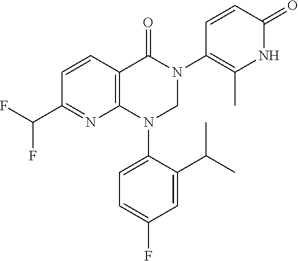 [SMILES: Cc1[nH]c(=O)ccc1N1CN(c2ccc(F)cc2C(C)C)c2nc(C(F)F)ccc2C1=O] | ¹HNMR (400 MHz, DMSO-d₆): δ = 11.81 (br s, 1H), 8.31 (d, J = 7.8 Hz, 1H), 7.46-7.27 (m, 3H), 7.19-7.09 (m, 2H), 6.81-6.50 (m, 1H), 6.24- 6.18 (m, 1H), 5.61 (d, J = 9.6 Hz, 0.6H), 5.26 (d, J = 10.0 Hz, 0.4H), 5.08 (d, J = 10.0 Hz, 0.4H), 4.79 (d, J = 9.6 Hz, 0.6H), 3.12-2.91 (m, 1H), 2.18-2.08 (m, 3H), 1.18-1.07 (m, 6H). MS (m/z) 443.1 (M + H)⁺ | 7- (difluoromethyl)- 1-(4-fluoro-2- isopropylphenyl)- 3-(6- methoxy-2- methylpyridin- 3-yl)-2,3- dihydropyrido[2,3- d]pyrimidin- 4(1H)-one |
| 123 | 3-methyl-4-(3-(2- methyl-6-oxo-1,6- dihydropyridin-3- yl)-4-oxo-6- (trifluoromethoxy)- 3,4- dihydroquinazolin- 1(2H)- yl)benzonitrile | 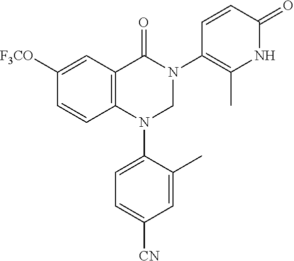 [SMILES: Cc1cc(C#N)ccc1N1CN(c2ccc(=O)[nH]c2C)C(=O)c2cc(OC(F)(F)F)ccc21] | ¹HNMR (400 MHz, DMSO-d₆): δ = 11.77 (br s, 1H), 7.91 (d, J = 1.5 Hz, 1H), 7.79-7.75 (m, 2H), 7.42 (dd, J = 8.6, 2.4 Hz, 2H), 7.35 (d, J = 9.5 Hz, 1H), 6.49 (brd, J = 6.8 Hz, 1H), 6.18 (d, J = 9.5 Hz, 1H), 5.36 (br s, 1H), 5.01 (br s, 1H), 2.24 (s, 3H), 2.03 (s, 3H). MS (m/z) 455.1 (M + H)⁺ | 4-(3-(6- methoxy-2- methylpyridin- 3-yl)-4-oxo-6- (trifluorometh- oxy)-3,4- dihydroquinazolin- 1(2H)-yl)-3- methylbenzonitrile |
| 124 | 8-chloro-1-(4- fluoro-2- methylphenyl)-3- (2-methyl-6-oxo- 1,6-dihydropyridin- 3-yl)-2,3- dihydroquinazolin- 4(1H)-one | 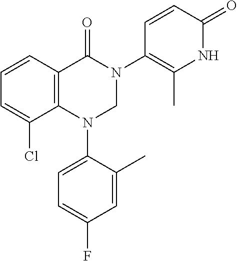 [SMILES: Cc1cc(F)ccc1N1CN(c2ccc(=O)[nH]c2C)C(=O)c2cccc(Cl)c21] | ¹HNMR (400 MHz, DMSO-d6, as a mixture of rotamers): δ 11.66 (s, 1H), 8.00 (d, J = 7.60 Hz, 1H), 7.66 (dd, J = 8.00, 1.20 Hz, 1H), 7.39 (d, J = 9.60 Hz, 1H), 7.32 (t, J = 7.60 Hz, 1H), 7.23-7.21 (m, 1H), 6.98-6.88 (m, 1H), 6.69-6.54 (m, 1H), 6.18-6.04 (m, 1H), 5.49- 5.42 (m, 1H), 4.60-4.50 (m, 1H), 2.44 (s, 3H), 2.10 (s, 1H), 1.62 (s, 2H). MS (m/z) 397.8 (M)⁺ | 8-chloro-1-(4- fluoro-2- methylphenyl)- 3-(6-methoxy- 2- methylpyridin- 3-yl)-2,3- dihydroquinazolin- 4(1H)-one |
| 125 | 1-(4-fluoro-2- methylphenyl)-8- methyl-3-(2- methyl-6-oxo-1,6- dihydropyridin-3- yl)-2,3- dihydroquinazolin- 4(1H)-one | 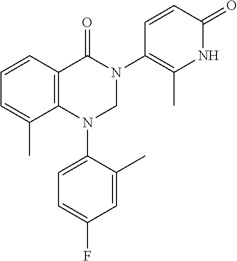 [SMILES: Cc1cc(F)ccc1N1CN(c2ccc(=O)[nH]c2C)C(=O)c2cccc(C)c21] | ¹HNMR (400 MHz, DMSO-d₆, 80° C., mixture of rotamers): δ: δ 11.32 (s, 1H), 7.90 (d, J = 7.60 Hz, 1H), 7.41-7.37 (m, 1.5 H), 7.24-7.17 (m, 2.5H), 6.91 (t, J = 6.40 Hz, 1H), 6.63-6.60 (m, 1 H), 6.12 (br s, 1H), 5.39 (br s, 1H), 4.51 (br s, 1H), 2.45 (s, 3H), 1.92- 48 (m, 6H). MS (m/z) 378.2 (M + H)⁺ | 1-(4-fluoro-2- methylphenyl)- 3-(6-methoxy- 2- methylpyridin- 3-yl)-8-methyl- 2,3- dihydroquinazolin- 4(1H)-one |
| 126 | 1-(4-methoxy-2- methylphenyl)-3- (2-methyl-6-oxo- 1,6-dihydropyridin- 3-yl)-6- (trifluoromethyl)- 2,3- dihydroquinazolin- 4(1H)-one | 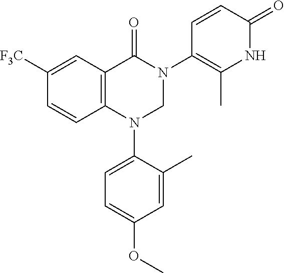 [SMILES: COc1ccc(N2CN(c3ccc(=O)[nH]c3C)C(=O)c3cc(C(F)(F)F)ccc32)c(C)c1] | ¹H NMR (400 MHz, DMSO-d₆) δ: 11.79 (s, 1H), 8.06 (s, 1H), 7.63 (dd, J = 2.00, 8.80 Hz, 1H), 7.39 (t, J = 11.60 Hz, 1H), 7.27 (d, J = 8.80 Hz, 1H), 7.01 (s, 1H), 6.94-6.87 (m, 1H), 6.34- 6.25 (m, 1H), 6.20 (d, J = 9.60 Hz, 1H), 5.52-4.71 (m, 2H), 3.79 (s, 3H), 2.20 (s, 3H), 2.13 (s, 3H). MS (m/z) 444.0 (M + H)⁺ | 1-(4-methoxy- 2- methylphenyl)- 3-(6-methoxy- 2- methylpyridin- 3-yl)-6- (trifluoromethyl)- 2,3- dihydroquinazolin- 4(1H)-one |
| 127 | 1-(4-hydroxy-2- methylphenyl)-3- (2-methyl-6-oxo- 1,6-dihydropyridin- 3-yl)-6- (trifluoromethyl)- 2,3- dihydroquinazolin- 4(1H)-one | 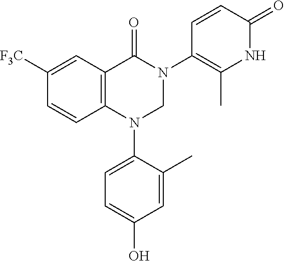 [SMILES: Cc1cc(O)ccc1N1CN(c2ccc(=O)[nH]c2C)C(=O)c2cc(C(F)(F)F)ccc21] | ¹H NMR (400 MHz, DMSO-d₆) δ: 11.79 (s, 1H), 9.66 (s, 1H), 8.04 (d, J = 2.00 Hz, 1H), 7.62 (dd, J = 2.00, 8.80 Hz, 1H), 7.42-7.32 (m, 1H), 7.13 (d, J = 8.40 Hz, 1H), 6.79 (s, 1H), 6.71-6.72 (m, 1H), 6.28-6.30 (m, 1H), 6.20 (d, J = 9.20 Hz, 1H), 4.68-4.71 (m, 2H), 2.13 (d, J = 4.40 Hz, 6H). MS (m/z) 430.0 (M + H)⁺ | 1-(4-methoxy- 2- methylphenyl)- 3-(6-methoxy- 2- methylpyridin- 3-yl)-6- (trifluoromethyl)- 2,3- dihydroquinazolin- 4(1H)-one |
| 128 | 3-(6-chloro-3-(2- methyl-6-oxo-1,6- dihydropyridin-3- yl)-4-oxo-3,4- dihydroquinazolin- 1(2H)-yl)-2- methylbenzonitrile | 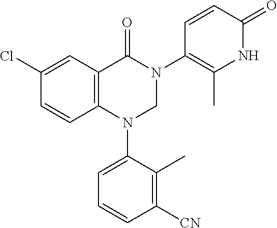 [SMILES: Cc1[nH]c(=O)ccc1N1CN(c2cccc(C#N)c2C)c2ccc(Cl)cc2C1=O] | ¹H NMR (400 MHz, DMSO-d₆) δ: 11.77 (s, 1H), 7.84-7.79 (m, 2H), 7.68-7.49 (m, 2H), 7.42- 7.31 (m, 2H), 6.50-6.25 (m, 1H), 6.18 (d, J = 9.20 Hz, 1H), 5.49-4.79 (m, 2H), 2.40 (s, 3H), 2.12- 1.89 (m, 3H). MS (m/z) 405.0 (M + H)⁺ | 3-(6-chloro-3- (6-methoxy-2- methylpyridin- 3-yl)-4-oxo- 3,4- dihydroquinazolin- 1(2H)-yl)-2- methylbenzonitrile |
| 129 | 6-fluoro-1-(4- fluoro-2- methylphenyl)-3- (2-methyl-6-oxo- 1,6-dihydropyridin- 3-yl)-7- (trifluoromethoxy)- 2,3- dihydroquinazolin- 4(1H)-one | 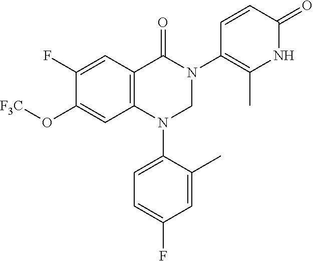 [SMILES: Cc1cc(F)ccc1N1CN(c2ccc(=O)[nH]c2C)C(=O)c2cc(F)c(OC(F)(F)F)cc21] | ¹H NMR (400 MHz, DMSO-d₆, as a mixture of rotamers) δ: 11.77 (s, 1H), 7.87 (d, J = 10.40 Hz, 1H), 7.41-7.26 (m, 3H), 7.22-7.12 (m, 1H), 6.35-6.15 (m, 2H), 5.50- 4.72 (m, 2H), 2.23 (s, 3H), 2.08 (s, 3H). MS (m/z) 466.0 (M + H)⁺ | 6-fluoro-1-(4- fluoro-2- methylphenyl)- 3-(6-methoxy- 2- methylpyridin- 3-yl)-7- (trifluorometh- oxy)-2,3- dihydroquinazolin- 4(1H)-one |
| 130 | 5-(difluoromethyl)- 1-(4-fluoro-2- methylphenyl)-3- (2-methyl-6-oxo- 1,6-dihydropyridin- 3-yl)-2,3- dihydroquinazolin- 4(1H)-one | 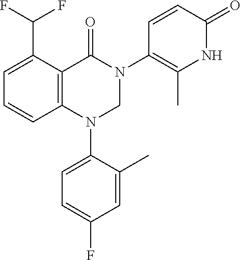 [SMILES: Cc1cc(F)ccc1N1CN(c2ccc(=O)[nH]c2C)C(=O)c2c(C(F)F)cccc21] | ¹H NMR (400 MHz, DMSO-d₆) δ: 11.77 (s, 1H), 7.63-7.77 (m, 1H), 7.45-7.47 (m, 1H), 7.25- 7.27 (m, 4H), 7.14-7.15 (m, 1H), 6.44 (s, 1H), 6.18 (d, J = 9.60 Hz, 1H), 4.80-5.02 (m, 2H), 2.23 (s, 3H), 2.09 (s, 3H). MS (m/z) 414.0 (M + H)⁺ | 5- (difluoromethyl)- 1-(4-fluoro-2- methylphenyl)- 3-(6-methoxy- 2- methylpyridin- 3-yl)-2,3- dihydroquinazolin- 4(1H)-one |
| 131 | 3-(5-fluoro-2- methyl-6-oxo-1,6- dihydropyridin-3- yl)-1-(4-fluoro-2- methylphenyl)-6- (trifluoromethyl)- 2,3- dihydroquinazolin- 4(1H)-one | 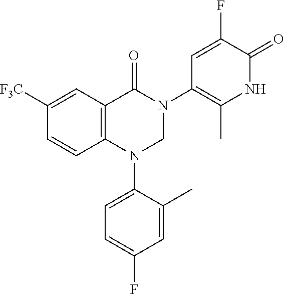 [SMILES: Cc1cc(F)ccc1N1CN(c2cc(F)c(=O)[nH]c2C)C(=O)c2cc(C(F)(F)F)ccc21] | ¹H NMR (400 MHz, DMSO-d₆) δ: 12.36 (s, 1H), 8.08 (d, J = 2.00 Hz, 1H), 7.66 (dd, J = 2.00, 8.80 Hz, 1H), 7.54-7.49 (m, 1H), 7.42-7.32 (m, 2H), 7.22-7.19 (m, 1H), 6.38-6.31 (m, 1H), 5.51- 4.79 (m, 2H), 2.23 (s, 3H), 2.11 (s, 3H). MS (m/z) 450.0 (M + H)⁺ | 1-(4-fluoro-2- methylphenyl)- 3-(5-fluoro-6- methoxy-2- methylpyridin- 3-yl)-6- (trifluoromethyl)- 2,3- dihydroquinazolin- 4(1H)-one |
| 132 | 6,7-difluoro-1-(4- fluoro-2- methoxyphenyl)-3- (2-methyl-6-oxo- 1,6-dihydropyridin- 3-yl)-2,3- dihydroquinazolin- 4(1H)-one | 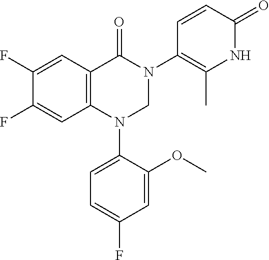 [SMILES: COc1cc(F)ccc1N1CN(c2ccc(=O)[nH]c2C)C(=O)c2cc(F)c(F)cc21] | ¹H NMR (400 MHz, DMSO-d6): δ 11.76 (s, 1H), 7.77 (dd, J = 10.60, 9.20 Hz, 1H), 7.35-7.30 (m, 2H), 7.13 (dd, J = 11.20, 2.80 Hz, 1H), 6.86 (td, J = 8.40, 2.80 Hz, 1H), 6.37 (dd, J = 12.00, 6.40 Hz, 1H), 6.17 (d, J = 9.60 Hz, 1H), 5.21 (d, J = 10.80 Hz, 1H), 4.81 (d, J = 10.80 Hz, 1H), 3.79 (s, 3H), 2.05 (s, 3H). MS (m/z) 416 (M + H)⁺ | 6,7-difluoro-1- (4-fluoro-2- methoxyphenyl)- 3-(6- methoxy-2- methylpyridin- dihydroquinazolin- 4(1H)-one |
| 133 | 6-chloro-1-(2- hydroxy-4- methylphenyl)-3- (2-methyl-6-oxo- 1,6-dihydropyridin- 3-yl)-2,3- dihydroquinazolin- 4(1H)-one | 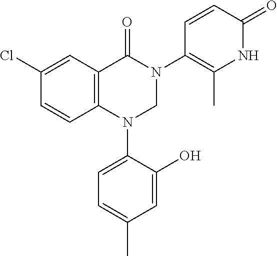 [SMILES: Cc1ccc(N2CN(c3ccc(=O)[nH]c3C)C(=O)c3cc(Cl)ccc32)c(O)c1] | ¹H NMR (400 MHz, DMSO-d6): δ 11.75 (s, 1H), 9.73 (s, 1H), 7.74 (s, 1H), 7.35-7.35 (m, 2H), 7.06 (d, J = 8.00 Hz, 1H), 6.80 (d, J = 1.20 Hz, 1H), 6.69 (q, J = 1.20 Hz, 1H), 6.40 (d, J = 8.80 Hz, 1H), 6.18 (d, J = 9.60 Hz, 1H), 5.18 (d, J = 10.00 Hz, 1H), 4.80 (d, J = 10.00 Hz, 1H), 2.26 (s, 3H), 2.08 (s, 3H). MS (m/z) 396 (M + H)⁺ | 6-chloro-3-(6- methoxy-2- methylpyridin- 3-yl)-1-(2- methoxy-4- methylphenyl)- 2,3- dihydroquinazolin- 4(1H)-one |
| 134 | 6-chloro-1-(2- methoxy-4- methylphenyl)-3- (2-methyl-6-oxo- 1,6-dihydropyridin- 3-yl)-2,3- dihydroquinazolin- 4(1H)-one | 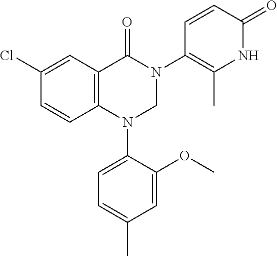 [SMILES: COc1cc(C)ccc1N1CN(c2ccc(=O)[nH]c2C)C(=O)c2cc(Cl)ccc21] | ¹H NMR (400 MHz, DMSO-d₆): δ 11.76 (s, 1H), 7.75 (d, J = 2.40 Hz, 1H), 7.35 (dd, J = 2.80, 8.80 Hz, 2H), 7.13 (d, J = 8.00 Hz, 1H), 7.02 (s, 1H), 6.84 (d, J = 7.20 Hz, 1H), 6.38 (d, J = 9.20 Hz, 1H), 6.17 (d, J = 9.60 Hz, 1H), 5.20 (d, J = 10.40 Hz, 1H), 4.78 (d, J = 10.40 Hz, 1H), 3.75 (s, 3H), 2.35 (s, 3H), 2.06 (s, 3H). MS (m/z) 410 (M + H)⁺ | 6-chloro-3-(6- methoxy-2- methylpyridin- 3-yl)-1-(2- methoxy-4- methylphenyl)- 2,3- dihydroquinazolin- 4(1H)-one |
| 135 | 1-(2-fluoro-6- methylphenyl)-3- (2-methyl-6-oxo- 1,6-dihydropyridin- 3-yl)-6- (trifluoromethyl)- 2,3- dihydroquinazolin- 4(1H)-one | 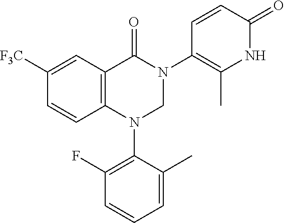 [SMILES: Cc1cccc(F)c1N1CN(c2ccc(=O)[nH]c2C)C(=O)c2cc(C(F)(F)F)ccc21] | ¹H NMR (400 MHz, DMSO-d₆): δ 11.80 (s, 1H), 8.11 (d, J = 2.00 Hz, 1H), 7.68 (d, J = 8.80 Hz, 1 H), 7.39-7.37 (m, 2H), 7.27-7.25 (m, 2H), 6.36- 6.34 (m, 1H), 6.20 (d, J = 9.60 Hz, 1H), 5.27-5.25 (m, 1H), 4.87-4.84 (m, 1H), 2.27 (s, 3H), 2.12 (s, 3H). MS (m/z) 432 (M + H)⁺ | 1-(2-fluoro-6- methylphenyl)- 3-(6-methoxy- 2- methylpyridin- 3-yl)-6- (trifluoromethyl)- 2,3- dihydroquinazolin- 4(1H)-one |
| 136 | 6-chloro-5-fluoro- 1-(4-fluoro-2- methylphenyl)-3- (2-oxo-1,2- dihydropyridin-4- yl)-2,3- dihydroquinazolin- 4(1H)-one | 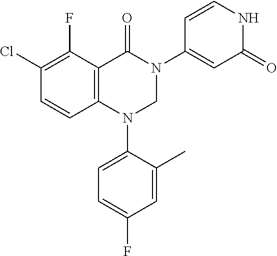 [SMILES: Cc1cc(F)ccc1N1CN(c2cc[nH]c(=O)c2)C(=O)c2c1ccc(Cl)c2F] | ¹H NMR (400 MHz, DMSO-d₆): δ 11.47 (s, 1H), 7.52 (dd, J = 8.20, 7.60 Hz, 1H), 7.35-7.27 (m, 3H), 7.16 (td, J = 8.40, 2.80 Hz, 1H), 6.36 (dd, J = 7.20, 2.00 Hz, 1H), 6.22 (d, J = 2.40 Hz, 1H), 6.16 (d, J = 8.40 Hz, 1H), 5.32-5.11 (m, 2H), 2.18 (s, 3H). MS (m/z) 402 (M + H)⁺ | 6-chloro-5- fluoro-1-(4- fluoro-2- methylphenyl)- 3-(2- methoxypyridin- 4-yl)-2,3- dihydroquinazolin- 4(1H)-one |
| 137 | 7-(difluoromethyl)- 1-(4-fluoro-2- isopropylphenyl)- 3-(2-methyl-6-oxo- 1,6-dihydropyridin- 3-yl)-2,3- dihydroquinazolin- 4(1H)-one | 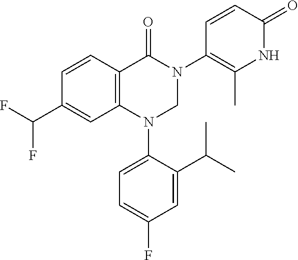 [SMILES: Cc1[nH]c(=O)ccc1N1CN(c2ccc(F)cc2C(C)C)c2cc(C(F)F)ccc2C1=O] | ¹H NMR (400 MHz, DMSO-d₆) 11.79 (br s, 1H), 7.98 (d, J = 8.0 Hz, 1H), 7.43-7.27 (m, 3H), 7.25-7.15 (m, 1H), 7.10- 7.06 (m, 1H), 6.94 (t, J = 56.0 Hz, 1H), 6.36- 6.26 (m, 1H), 6.20 (d, J = 9.5 Hz, 1H), 5.51 (d, J = 9.3 Hz, 0.6H), 5.21 (d, J = 10.4 Hz, 0.4H), 4.95 (d, J = 9.8 Hz, 0.4H), 4.66 (d, J = 9.3 Hz, 0.6H), 3.24-3.02 (m, 1H), 2.13 (s, 3H), 1.23-1.05 (m, 6H). | 7- (difluoromethyl)- 1-(4-fluoro-2- isopropylphenyl)- 3-(6- methoxy-2- methylpyridin- 3-yl)-2,3- dihydroquinazolin- 4(1H)-one |
| | | | MS (m/z) 442.1 (M + H)⁺ | |
| 138 | 7-(difluoromethyl)- 1-(4-fluoro-2- methylphenyl)-3- (2-methyl-6-oxo- 1,6-dihydropyridin- 3-yl)-2,3- dihydroquinazolin- 4(1H)-one | 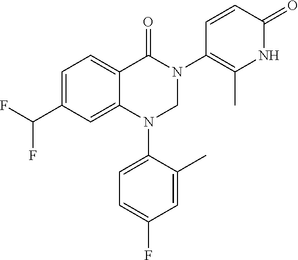 [SMILES: Cc1cc(F)ccc1N1CN(c2ccc(=O)[nH]c2C)C(=O)c2ccc(C(F)F)cc21] | ¹H NMR (400 MHz, DMSO-d₆) δ: 11.77 (br s, 1H), 7.99 (d, J = 8.0 Hz, 1H), 7.40-7.28 (m, 3H), 7.23-7.14 (m, 1H), 7.10 (d, J = 8.1 Hz, 1H), 6.94 (t, J = 55.7 Hz, 1H), 6.47- 6.28 (m, 1H), 6.19 (d, J = 9.5 Hz, 1H), 5.53- 5.37 (m, 0.5H), 5.21- 4.98 (m, 1H), 4.83-4.66 (m, 0.5H), 2.23 (s, 3H), 2.10 (br s, 3H) MS (m/z) 414.0 (M + H)⁺ | 7- (difluoromethyl)- 1-(4-fluoro-2- methylphenyl)- 3-(6-methoxy-2- methylpyridin- 3-yl)-2,3- dihydroquinazolin- 4(1H)-one |
| 139 | 6-chloro-3-(2- ethyl-6-oxo-1,6- dihydropyridin-3- yl)-1-(4-fluoro-2- isopropylphenyl)- 2,3- dihydroquinazolin- 4(1H)-one | 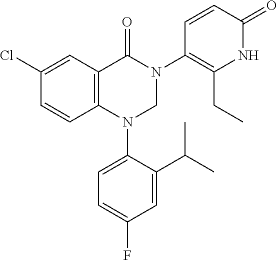 [SMILES: CCc1[nH]c(=O)ccc1N1CN(c2ccc(F)cc2C(C)C)c2ccc(Cl)cc2C1=O] | ¹H NMR (400 MHz, DMSO-d₆) δ: 12.17 (brs, 1H), 8.06 (t, J = 2.2 Hz, 1H), 7.33 (d, J = 9.5 Hz, 1H), 7.28-7.23 (m, 1H), 7.18-7.11 (m, 2H), 7.04- 6.96 (m, 1H), 6.48 (d, J = 9.5 Hz, 1H), 6.31-6.19 (m, 1H), 5.32 (d, J = 9.3 Hz, 0.5H), 5.00 (d, J = 9.9 Hz, 0.5H), 4.88 (d, J = 9.9 Hz, 0.5H), 4.55 (d, J = 9.3 Hz, 0.5H), 3.38- 3.10 (m, 1H), 2.81-2.65 (m, 1H), 2.64-2.55 (m, 1H), 1.38-1.14 (m, 9H). | 6-chloro-3-(2- ethyl-6- methoxypyridin- 3-yl)-1-(4- fluoro-2- isopropylphenyl)- 2,3- dihydroquinazolin- 4(1H)-one |
| | | | MS (m/z) 440.0 (M + H)⁺ | |
| 140 | 6-fluoro-2-methyl- 3-(3-(2-methyl-6- oxo-1,6- dihydropyridin-3- yl)-4-oxo-6- (trifluoromethyl)- 3,4- dihydroquinazolin- 1(2H)- yl)benzonitrile | 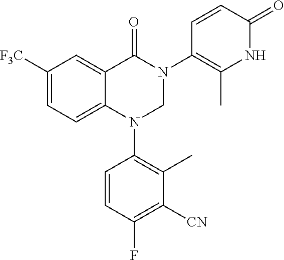 [SMILES: Cc1[nH]c(=O)ccc1N1CN(c2ccc(F)c(C#N)c2C)c2ccc(C(F)(F)F)cc2C1=O] | ¹H NMR (400 MHz, DMSO-d₆) δ: 11.80 (brs, 1H), 8.10 (d, J = 1.8 Hz, 1H), 7.85-7.77 (m, 1H), 7.66 (br d, J = 8.6 Hz, 1H), 7.60-7.49 (m, 1H), 7.39 (d, J = 9.5 Hz, 1H), 6.57-6.43 (m, 1H), 6.20 (d, J = 9.4 Hz, 1H), 5.51 (d, J = 9.6 Hz, 0.6H), 5.25-5.20 (m, 0.4H), 5.16-5.09 (m, 0.4H), 4.84 (d, J = 9.6 Hz, 0.6H), 2.44-2.38 (m, 3H), 2.15-2.06 (m, 3H). MS (m/z) 457.2 (M + H)⁺ | 6-fluoro-3-(3- (6-methoxy-2- methylpyridin- 3-yl)-4-oxo-6- (trifluoromethyl)- 3,4- dihydroquinazolin- 1(2H)-yl)-2- methylbenzonitrile |
| 141 | 6-fluoro-1-(4- fluoro-2- isopropylphenyl)- 3-(2-methyl-6-oxo- 1,6-dihydropyridin- 3-yl)-2,3- dihydropyrido[2,3- d]pyrimidin-4(1H)- one | 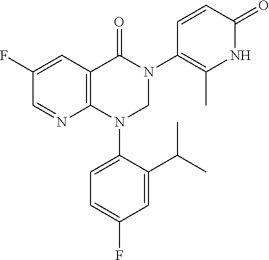 [SMILES: Cc1[nH]c(=O)ccc1N1CN(c2ccc(F)cc2C(C)C)c2ncc(F)cc2C1=O] | ¹H NMR (400 MHz, DMSO-d₆) δ: 11.81 (brs, 1H), 8.27 (d, J = 3.0 Hz, 1H), 8.00 (dd, J = 8.1, 3.1 Hz, 1H), 7.44-7.24 (m, 3H), 7.10 (td, J = 8.4, 3.0 Hz, 1H), 6.21 (d, J = 9.5 Hz, 1H), 5.55 (d, J = 9.6 Hz, 0.6H), 5.26 (d, J = 10.1 Hz, 0.4H), 5.00 (d, J = 10.1 Hz, 0.4H), 4.74 (d, J = 9.5 Hz, 0.6H), 3.18-3.00 (m, 1H), 2.18- 2.07 (m, 3H), 1.23- 1.02 (m, 6H). MS (m/z) 411.2 (M + H)⁺ | 6-fluoro-1-(4- fluoro-2- isopropylphenyl)- 3-(6- methoxy-2- methylpyridin- 3-yl)-2,3- dihydropyrido[2,3- d]pyrimidin- 4(1H)-one |
| 142 | 1-(2-bromo-4- fluorophenyl)-3-(2- methyl-6-oxo-1,6- dihydropyridin-3- yl)-6- (trifluoromethyl)- 2,3- dihydroquinazolin- 4(1H)-one | 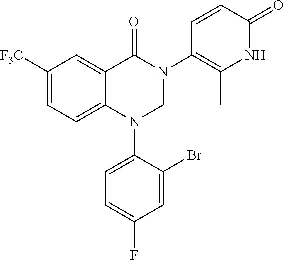 [SMILES: Cc1[nH]c(=O)ccc1N1CN(c2ccc(F)cc2Br)c2ccc(C(F)(F)F)cc2C1=O] | ¹H NMR (400 MHz, DMSO-d₆) δ: 11.81 (s, 1H), 8.09 (s, 1H), 7.87 (dd, J = 2.80, 8.20 Hz, 1H), 7.70-7.63 (m, 2H), 7.48-7.37 (m, 2H), 6.47- 6.38 (m, 1H), 6.21 (d, J = 9.60 Hz, 1H), 5.56-4.82 (m, 2H), 2.15-2.11 (m, 3H) MS (m/z) 495.8 (M + H)⁺ | 1-(2-bromo-4- fluorophenyl)- 3-(6-methoxy- 2- methylpyridin- 3-yl)-6- (trifluoromethyl)- 2,3- dihydroquinazolin- 4(1H)-one |
| 143 | 6-fluoro-1-(4- fluoro-2- methylphenyl)-3- (2-methyl-6-oxo- 1,6-dihydropyridin- 3-yl)-2,3- dihydroquinazolin- 4(1H)-one | 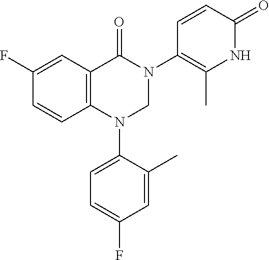 [SMILES: Cc1cc(F)ccc1N1CN(c2ccc(=O)[nH]c2C)C(=O)c2cc(F)ccc21] | ¹HNMR (400 MHz, DMSO-d₆): δ 11.76 (br s, 1H), 7.58 (dd, J = 8.9, 3.0 Hz, 1H), 7.34 (d, J = 9.5 Hz, 1H), 7.30-7.21 (m, 3H), 7.17-7.09 (m, 1H), 6.31 (br s, 1H), 6.18 (d, J = 9.5 Hz, 1H), 5.27 (br s, 1H), 4.82 (br s, 1H), 2.24 (s, 3H), 2.06 (br s, 3H). MS (m/z) 382.2 (M + H)⁺ | 6-fluoro-1-(4- fluoro-2- methylphenyl)- 3-(6-methoxy- 2- methylpyridin- 3-yl)-2,3 dihydroquinazolin- 4(1H)-one |

### Example 144

### 1-(4-Fluorophenyl)-3-(6-oxo-1,6-dihydropyridin-3-yl)-7-(trifluoromethyl)-2,3-dihydroquinazolin-4(1H)-one

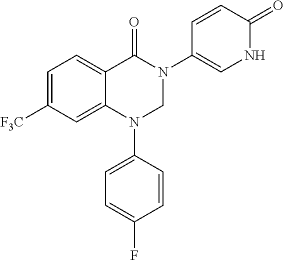 [SMILES: O=C1c2ccc(C(F)(F)F)cc2N(c2ccc(F)cc2)CN1c1ccc(=O)[nH]c1]

To a mixture of 1-(4-fluorophenyl)-3-(6-methoxypyridin-3-yl)-7-(trifluoromethyl)-2,3-dihydroquinazolin-4(1H)-one (150 mg, 0.359 mmol) and sodium iodide (354 mg, 2.365 mmol) in acetonitrile (1.50 mL) was added TMS-Cl (0.300 mL, 2.364 mmol) and the reaction was stirred at 55° C. for 1.5 hour. The reaction was diluted with brine and extracted with EtOAc. The organic layer was extracted with EtOAc, washed with 1N HCl and brine, dried over Na₂SO₄, filtered and concentrated under reduced pressure. The crude product was purified by reverse phase column chromatography (EZ Prep Isco, 50 g Aq C18, 20-85% gradient, acetonitrile with 0.1% formic acid/water with 0.1% formic acid, 40 ml/min flow rate, 25 min overall run time) to give the title compound as an off-white solid (96 mg, 0.226 mmol, 62.9% yield). ¹H NMR (400 MHz, DMSO-d₆) δ: 11.71 (br. s., 1H), 8.09 (d, J=8.11 Hz, 1H), 7.50-7.30 (m, 7H), 6.95 (s, 1H), 6.35 (d, J=9.63 Hz, 1H), 5.31 (s, 2H). MS (m/z) 404.3 (M+H)⁺.

Examples 145-214 were prepared from the indicated intermediate by methods analogous to those described for Example 144.

| Ex. | Name | Structure | Characterization | Intermediate |
|---|---|---|---|---|
| 145 | 1-(4-fluoro-2- methylphenyl)-3- (2-oxo-1,2- dihydropyrimidin- 5-yl)-7- (trifluoromethyl)- 2,3- dihydroquinazolin- 4(1H)-one | 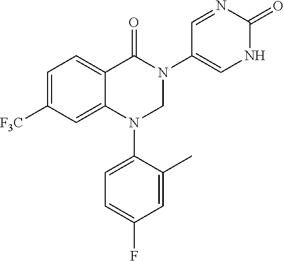 [SMILES: Cc1cc(F)ccc1N1CN(c2cnc(=O)[nH]c2)C(=O)c2ccc(C(F)(F)F)cc21] | ¹H NMR (400 MHz, DMSO-d₆) δ: 8.40 (s, 2 H), 8.09 (d, J = 8.07 Hz, 1H), 7.40 (dd, J = 8.80, 5.38 Hz, 1H), 7.33 (dd, J = 9.66, 2.81 Hz, 1H), 7.15-7.29 (m, 2H), 6.39 (s, 1H), 5.40 (br. s., 1H), 5.14 (br. s., 1 H), 2.24 (s, 3H). MS (m/z) 419.2 (M + H)⁺ | 1-(4-fluoro-2- methylphenyl)- 3-(2- methoxypyrimidin- 5-yl)-7- (trifluoromethyl)- 2,3- dihydroquinazolin- 4(1H)-one |
| 146 | 1-(4-bromo-2- methylphenyl)-3- (6-oxo-1,6- dihydropyridin-3- yl)-7- (trifluoromethyl)- 2,3- dihydroquinazolin- 4(1H)-one | 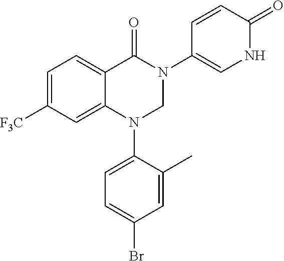 [SMILES: Cc1cc(Br)ccc1N1CN(c2ccc(=O)[nH]c2)C(=O)c2ccc(C(F)(F)F)cc21] | ¹H NMR (400 MHz, DMSO-d₆) δ: 11.72 (br. s., 1H), 8.08 (d, J = 8.07 Hz, 1H), 7.68 (d, J = 1.96 Hz, 1H), 7.45- 7.57 (m, 3 H), 7.28 (d, J = 8.56 Hz, 2 H), 6.44 (s, 1H), 6.36 (d, J = 9.54 Hz, 1H), 5.28 (br. s., 1 H), 5.16 (br. s., 1H), 2.21 (s, 3H) MS (m/z) 478.1 (M + H)⁺ | 1-(4-bromo-2- methylphenyl)- 3-(6- methoxypyridin- 3-yl)-7- (trifluoromethyl)- 2,3- dihydroquinazolin- 4(1H)-one |
| 147 | 1-(4- fluorophenyl)-3- (2-methyl-6-oxo- 1,6- dihydropyridin-3- yl)-7- (trifluoromethyl)- 2,3- dihydroquinazolin- 4(1H)-one | 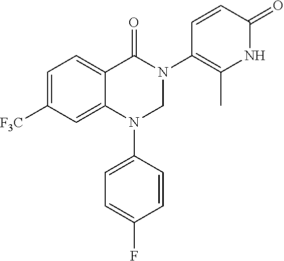 [SMILES: Cc1[nH]c(=O)ccc1N1CN(c2ccc(F)cc2)c2cc(C(F)(F)F)ccc2C1=O] | ¹H NMR (400 MHz, DMSO-d₆) δ: 11.77 (br. s., 1H), 8.09 (d, J = 7.82 Hz, 1H), 7.39-7.27 (m, 6H), 6.99 (s, 1H), 6.18 (d, J = 9.54 Hz, 1H), 5.34 (d, J = 10.76 Hz, 1H), 5.11 (d, J = 10.76 Hz, 1H), 1.98 (s, 3H). MS (m/z) 418.3 (M + H)⁺ | 1-(4- fluorophenyl)-3- (6-methoxy-2- methylpyridin-3- yl)-7- (trifluoromethyl)- 2,3- dihydroquinazolin- 4(1H)-one |
| 148 | 3-methyl-4-(3-(2- methyl-6-oxo- 1,6- dihydropyridin-3- yl)-4-oxo-7- (trifluoromethyl)- 3,4- dihydroquinazolin- 1(2H)- yl)benzonitrile | 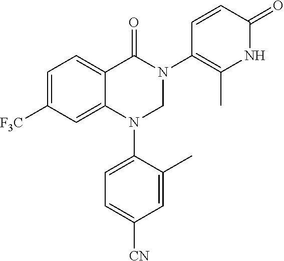 [SMILES: Cc1cc(C#N)ccc1N1CN(c2ccc(=O)[nH]c2C)C(=O)c2ccc(C(F)(F)F)cc21] | ¹H NMR (400 MHz, DMSO-d₆) δ: 11.78 (br. s., 1H), 8.11 (d, J = 7.82 Hz, 1H), 7.89-7.98 (m, 1H), 7.80 (d, J = 8.07 Hz, 1H), 7.45 (d, J = 8.07 Hz, 1H), 7.36 (d, J = 9.54 Hz, 2 H), 6.58 (br. s., 1H), 6.19 (d, J = 9.54 Hz, 1H), 5.40 (br. s., 1H), 5.05 (br. s., 1H), 2.24 (s, 3 H), 2.03 (br. s., 3H). MS (m/z) 439.3 (M + H)⁺ | 4-(3-(6- methoxy-2- methylpyridin-3- yl)-4-oxo-7- (trifluoromethyl)- 3,4- dihydroquinazolin- 1(2H)-yl)-3-methyl- benzonitrile |
| 149 | 1-(4-fluoro-2- methylphenyl)-3- (2-methyl-6-oxo- 1,6- dihydropyridin-3- yl)-7- (trifluoromethyl)- 2,3- dihydropyrido[2, 3-d]pyrimidin- 4(1H)-one | 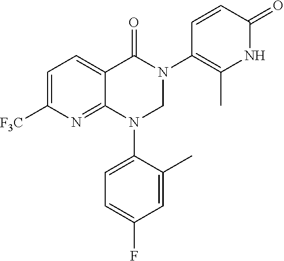 [SMILES: Cc1cc(F)ccc1N1CN(c2ccc(=O)[nH]c2C)C(=O)c2ccc(C(F)(F)F)nc21] | ¹H NMR (400 MHz, DMSO-d₆) δ: 11.83 (br. s., 1H), 8.37 (d, J = 7.58 Hz, 1H), 7.39-7.52 (m, 1H), 7.35 (d, J = 7.83 Hz, 2H), 7.24 (dd, J = 9.66, 2.81 Hz, 1H), 7.13 (td, J = 8.44, 2.93 Hz, 1H), 6.22 (d, J = 8.80 Hz, 1H), 5.62 (d, J = 8.80 Hz, 0.6H), 5.33 (d, J = 8.80 Hz, 0.4 H), 5.14 (d, J = 8.80 Hz, 0.4H), 4.89 (d, J = 9.29 Hz, 0.6H), 2.18 (s, 3H), 2.13 (d, J = 12.23 Hz, 3H). MS (m/z) 433.3 (M + H)⁺ | 1-(4-fluoro-2- methylphenyl)- 3-(6-methoxy-2- methylpyridin-3- yl)-7- (trifluoromethyl)- 2,3- dihydropyrido[2, 3-d]pyrimidin- 4(1H)-one |
| 150 | 3-(2-ethyl-6-oxo- 1,6- dihydropyridin-3- yl)-1-(4-fluoro-2- methylphenyl)-7- (trifluoromethyl)- 2,3- dihydroquinazolin- 4(1H)-one | 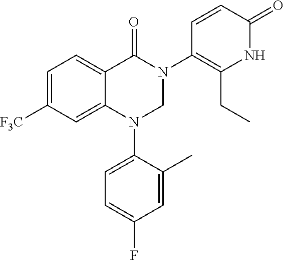 [SMILES: CCc1[nH]c(=O)ccc1N1CN(c2ccc(F)cc2C)c2cc(C(F)(F)F)ccc2C1=O] | ¹H NMR (400 MHz, CD₃OD) δ: 8.14 (d, J = 8.07 Hz, 1H), 7.54 (d, J = 8.80 Hz, 1H), 7.31 (br. s., 1H), 7.23- 7.20 (m, 2 H), 7.11 (br. s., 1H), 6.55-6.42 (m, 2 H), 5.58-4.78 (m, 2H), 2.63-2.56 (m, 2H), 2.30 (br. s., 3H), 1.19-1.17 (m, 3H). MS (m/z) 446.2 (M + H)⁺ | 3-(2-ethyl-6- methoxypyridin- 3-yl)-1-(4- fluoro-2- methylphenyl)-7- (trifluoromethyl)- 2,3- dihydroquinazolin- 4(1H)-one |
| 151 | 3-(2-chloro-6- oxo-1,6- dihydropyridin-3- yl)-1-(4-fluoro-2- methylphenyl)-7- (trifluoromethyl)- 2,3- dihydroquinazolin- 4(1H)-one | 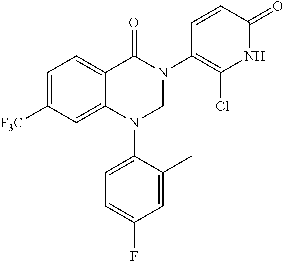 [SMILES: Cc1cc(F)ccc1N1CN(c2ccc(=O)[nH]c2Cl)C(=O)c2ccc(C(F)(F)F)cc21] | ¹H NMR (400 MHz, DMSO-d₆) δ: 11.79 (br. s., 1H), 8.08 (d, J = 8.07 Hz, 1H), 7.81 (d, J = 8.56 Hz, 1H), 7.40 (dd, J = 8.56, 5.62 Hz, 1 H), 7.31 (dd, J = 9.54, 2.69 Hz, 1H), 7.25 (d, J = 8.07 Hz, 1H), 7.19 (br. s., 1H), 6.71 (d, J = 8.56 Hz, 1H), 6.36 (br. s., 1H), 5.56-4.85 (m, 2H), 2.24 (s, 3H). MS (m/z) 452.1 (M + H)⁺ | 3-(2-chloro-6- methoxypyridin- 3-yl)-1-(4- fluoro-2- methylphenyl)-7- (trifluoromethyl)- 2,3- dihydroquinazolin- 4(1H)-one |
| 152 | 7-bromo-1-(4- fluoro-2- methylphenyl)-3- (2-methyl-6-oxo- 1,6- dihydropyridin-3- yl)-2,3- dihydroquinazolin- 4(1H)-one | 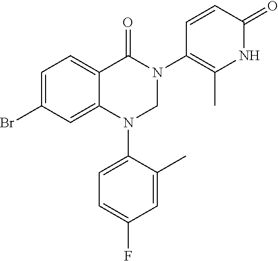 [SMILES: Cc1cc(F)ccc1N1CN(c2ccc(=O)[nH]c2C)C(=O)c2ccc(Br)cc21] | ¹H NMR (400 MHz, DMSO-d₆) δ: 11.76 (br. s., 1H), 7.78 (d, J = 8.31 Hz, 1H), 7.28-7.43 (m, 3H), 7.19 (br. s., 1H), 7.10 (dd, J = 8.31, 1.71 Hz, 1H), 6.24 (br. s., 1H), 6.18 (d, J = 9.54 Hz, 1H), 5.42 (br. s, 0.6H), 5.11 (br. s, 0.4H), 5.02 (br. s., 0.4H), 4.71 (br. s., 0.6H), 2.24 (s, 3H), 2.09 (d, J = 8.31 Hz, 3H). MS (m/z) 442.0 (M + H)⁺ | 7-bromo-1-(4- fluoro-2- methylphenyl)- 3-(6-methoxy-2- methylpyridin-3- yl)-2,3- dihydroquinazolin- 4(1H)-one |
| 153 | 1-(4-fluoro-2- methylphenyl)-3- (2-methyl-6-oxo- 1,6- dihydropyridin-3- yl)-4-oxo- 1,2,3,4-tetra- hydroquinazoline- 7-carbonitrile | 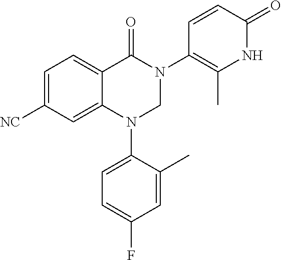 [SMILES: Cc1cc(F)ccc1N1CN(c2ccc(=O)[nH]c2C)C(=O)c2ccc(C#N)cc21] | ¹H NMR (400 MHz, CD₃OD) δ: 8.12 (d, J = 8.07 Hz, 1H), 7.67 (d, J = 9.29 Hz, 1H), 7.35 (dd, J = 8.68, 5.26 Hz, 1H), 7.26 (dd, J = 7.95, 1.34 Hz, 1H), 7.23 (dd, J = 9.54, 2.93 Hz, 1H), 7.05-7.18 (m, 1H), 6.55 (d, J = 9.29 Hz, 2H), 5.52 (br. s., 0.6H), 5.20 (br. s., 0.8H), 4.88 (br. s., 0.6H), 2.33 (s, 3H), 2.32 (s, 3H). MS (m/z) 389.1 (M + H)⁺ | 1-(4-fluoro-2- methylphenyl)- 3-(6-methoxy-2- methylpyridin-3- yl)-4-oxo- 1,2,3,4-tetra- hydroquinazoline- 7-carbonitrile |
| 154 | 3-(1-(4-fluoro-2- methylphenyl)-4- oxo-7- (trifluoromethyl)- 1,4- dihydroquinazolin- 3(2H)-yl)-6- oxo-1,6- dihydropyridine- 2-carbonitrile | 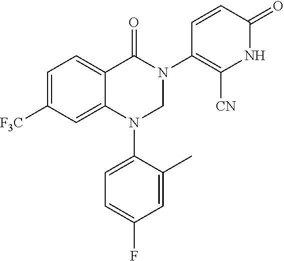 [SMILES: Cc1cc(F)ccc1N1CN(c2ccc(=O)[nH]c2C#N)C(=O)c2ccc(C(F)(F)F)cc21] | ¹H NMR (400 MHz, DMSO-d₆) δ: 12.06 (br. s., 1H), 8.12 (d, J = 8.07 Hz, 1H), 7.92 (d, J = 9.05 Hz, 1H), 7.40 (dd, J = 8.80, 5.38 Hz, 1H), 7.26-7.37 (m, 2H), 7.17-7.25 (m, 1H), 7.04-7.12 (m, 1H), 6.43 (s, 1H), 5.55 (br. s., 1H), 5.17 (br. s., 1H), 2.25 (s, 3H). MS (m/z) 443.0 (M + H)⁺ | 3-(1-(4-fluoro-2- methylphenyl)- 4-oxo-7- (trifluoromethyl)- 1,4- dihydroquinazolin- 3(2H)-yl)-6- methoxy- picolinonitrile |
| 155 | 1-(2,4- difluorophenyl)- 3-(2-methyl-6- oxo-1,6- dihydropyridin-3- yl)-7- (trifluoromethyl)- 2,3- dihydroquinazolin- 4(1H)-one | 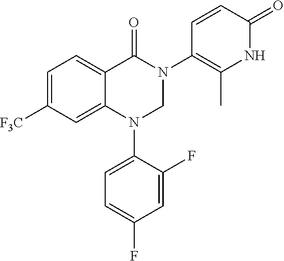 [SMILES: Cc1[nH]c(=O)ccc1N1CN(c2ccc(F)cc2F)c2cc(C(F)(F)F)ccc2C1=O] | ¹H NMR (400 MHz, DMSO-d₆) δ: 11.79 (br. s., 1H), 8.08 (d, J = 8.07 Hz, 1H), 7.57-7.51 (m, 2H), 7.38 (d, J = 9.54 Hz, 1H), 7.34-7.32 (m, 1H), 7.26-7.21 (m, 1H), 6.72 (s, 1H), 6.19 (d, J = 9.54 Hz, 1H), 5.36 (d, J = 10.51 Hz, 1H), 5.03 (d, J = 10.27 Hz, 1H), 2.06 (s, 3H). MS (m/z) 436.2 (M + H)⁺ | 1-(2,4- difluorophenyl)- 3-(6-methoxy-2- methylpyridin-3- yl)-7- (trifluoromethyl)- 2,3- dihydroquinazolin- 4(1H)-one |
| 156 | 1-(4- ethoxyphenyl)-3- (2-methyl-6-oxo- 1,6- dihydropyridin-3- yl)-7- (trifluoromethyl)- 2,3- dihydroquinazolin- 4(1H)-one | 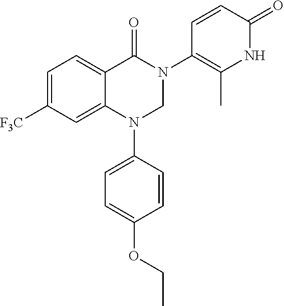 [SMILES: CCOc1ccc(N2CN(c3ccc(=O)[nH]c3C)C(=O)c3ccc(C(F)(F)F)cc32)cc1] | ¹H NMR (400 MHz, DMSO-d₆) δ: 11.76 (br. s., 1H) 8.05 (d, J = 8.07 Hz, 1H) 7.35 (d, J = 9.54 Hz, 1H) 7.26 (d, J = 9.05 Hz, 3H) 7.02 (d, J = 9.05 Hz, 2H) 6.82 (s, 1H) 6.18 (d, J = 9.54 Hz, 1H) 5.29 (d, J = 10.27 Hz, 1H) 5.02 (d, J = 10.27 Hz, 1H) 4.05 (q, J = 7.01 Hz, 2H) 2.03 (s, 3H) 1.33 (t, J = 6.97 Hz, 3H). MS (m/z) 444.3 (M + H)⁺ | 1-(4- ethoxyphenyl)- 3-(6-methoxy-2- methylpyridin-3- yl)-7- (trifluoromethyl)- 2,3- dihydroquinazolin- 4(1H)-one |
| 157 | 1-(4-fluoro-2- methylphenyl)-3- (2-oxo-1,2- dihydropyridin-4- yl)-7- (trifluoromethyl)- 2,3- dihydroquinazolin- 4(1H)-one | 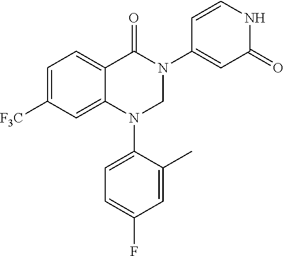 [SMILES: Cc1cc(F)ccc1N1CN(c2cc[nH]c(=O)c2)C(=O)c2ccc(C(F)(F)F)cc21] | ¹H NMR (400 MHz, DMSO-d₆) δ: 11.49 (br. s., 1H), 8.14 (d, J = 8.11 Hz, 1H), 7.41 (dd, J = 8.74, 5.45 Hz, 1H), 7.25-7.38 (m, 3H), 7.16-7.25 (m, 1H), 6.45, (s, 1H), 6.42 (dd, J = 7.35, 2.28 Hz, 1H), 6.26 (d, J = 2.28 Hz, 1 H), 5.42 (br. s., 1H), 5.26 (br. s., 1H), 2.21 (s, 3H). MS (m/z) 418.3 (M + H)⁺ | 1-(4-fluoro-2- methylphenyl)- 3-(2- methoxypyridin- 4-yl)-7- (trifluoromethyl)- 2,3- dihydroquinazolin- 4(1H)-one |
| 158 | 6,7-difluoro-1-(4- fluoro-2- methylphenyl)-3- (2-methyl-6-oxo- 1,6- dihydropyridin-3- yl)-2,3- dihydroquinazolin- 4(1H)-one | 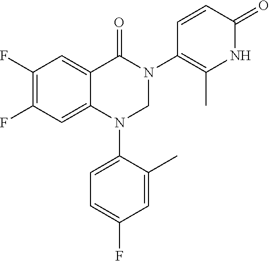 [SMILES: Cc1cc(F)ccc1N1CN(c2ccc(=O)[nH]c2C)C(=O)c2cc(F)c(F)cc21] | ¹H NMR (400 MHz, CHLOROFORM-d) δ: 7.90 (dd, J = 10.03, 8.80 Hz, 1H), 7.34 (d, J = 9.05 Hz, 1H), 7.19- 6.96 (m, 3H), 6.47 (d, J = 9.29 Hz, 1H), 6.12 (br s., 1H), 4.56-5.35 (m, 2H), 2.40-2.24(m, 6 H). MS (m/z) 400.2 (M + H)⁺ | 6,7-difluoro-1- (4-fluoro-2- methylphenyl)- 3-(6-methoxy-2- methylpyridin-3- yl)-2,3- dihydroquinazolin- 4(1H)-one |
| 159 | 6,7-difluoro-1-(4- fluoro-2- isopropylphenyl)- 3-(2-methyl-6- oxo-1,6- dihydropyridin-3- yl)-2,3- dihydroquinazolin- 4(1H)-one | 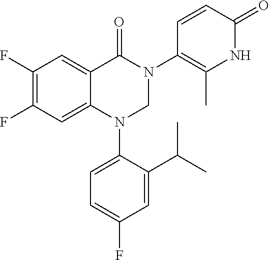 [SMILES: Cc1[nH]c(=O)ccc1N1CN(c2ccc(F)cc2C(C)C)c2cc(F)c(F)cc2C1=O] | ¹H NMR (400 MHz, CHLOROFORM-d) δ: 7.90 (dd, J = 10.03, 8.80 Hz, 1H), 7.35 (d, J = 9.54 Hz, 1H), 7.23-7.11 (m, 2 H), 7.02 (br s, 1H), 6.49 (d, J = 9.54 Hz, 1H), 6.07 (d, J = 6.85 Hz, 1H), 5.41- 4.46 (m, 2H), 3.38-3.16 (m, 1H), 2.45-2.29 (m, 3 H), 1.22 (d, J = 5.62 Hz, 6H). MS (m/z) 428.3 (M + H)⁺ | 6,7-difluoro-1- (4-fluoro-2- isopropylphenyl)- 3-(6-methoxy- 2-methylpyridin- 3-yl)-2,3- dihydroquinazolin- 4(1H)-one |
| 160 | 3-(2-methyl-6- oxo-1,6- dihydropyridin-3- yl)-1-((1S,2R)-2- methylcyclohexyl)- 7-(trifluoromethyl)- 2,3- dihydroquinazolin- 4(1H)-one | 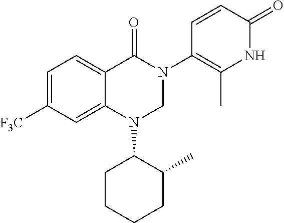 [SMILES: Cc1[nH]c(=O)ccc1N1CN([C@H]2CCCC[C@H]2C)c2cc(C(F)(F)F)ccc2C1=O] | ¹HNMR (400 MHz, CHLOROFORM-d) δ: 8.15-8.10 (m, 1H), 7.47-7.35 (m, 1H), 7.12 (br d, J = 8.3 Hz, 1H), 7.06 (s, 1H), 6.62- 6.53 (m, 1H), 4.98- 4.64 (m, 2H), 3.85- 3.73 (m, 1H), 2.48- 2.33 (m, 4H), 1.97- 1.81 (m, 2H), 1.77- 1.60 (m, 3H), 1.56- 1.39 (m, 3H), 1.01 (d, J = 6.8 Hz, 3H) MS (m/z) | 3-(6-methoxy-2- methylpyridin-3- yl)-1-((1S,2R)-2- methylcyclohexyl)- 7- (trifluoromethyl)- 2,3- dihydroquinazolin- 4(1H)-one |
| | | | 420.4 (M + H)⁺ | |
| 161 | 1-(4-fluoro-2- isopropylphenyl)- 3-(2-methyl-6- oxo-1,6- dihydropyridin-3- yl)-4-oxo- 1,2,3,4-tetra- hydroquinazoline- 7-carbonitrile | 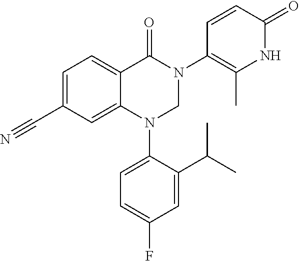 [SMILES: Cc1[nH]c(=O)ccc1N1CN(c2ccc(F)cc2C(C)C)c2cc(C#N)ccc2C1=O] | ¹H NMR (400 MHz, CHLOROFORM-d) δ: 8.17 (d, J = 8.3 Hz, 1H), 7.40-7.30 (m, 1H), 7.20- 7.10 (m, 3H), 7.10-7.00 (m, 1H), 6.60-6.40 (m, 2H), 5.39 (br d, J = 9.3 Hz, 0.6H), 5.13 (br d, J = 9.8 Hz, 0.4H), 4.87 (br d, J = 9.8 Hz, 0.4H), 4.59 (br d, J = 9.3 Hz, 0.6H), 3.1-3.4 (m, 1H), 2.3-2.4 (m, 3H), 1.2-1.4 (m, 6H). MS (m/z) 417.3 (M + H)⁺ | 1-(4-fluoro-2- isopropylphenyl)- 3-(6-methoxy- 2-methylpyridin- 3-yl)-4-oxo- 1,2,3,4-tetra- hydroquinazoline- 7-carbonitrile |
| 162 | 3-(2-methyl-6- oxo-1,6- dihydropyridin-3- yl)-1-((1R,2S)-2- methylcyclohexyl)- 7-(trifluoromethyl)- 2,3- dihydroquinazolin- 4(1H)-one | 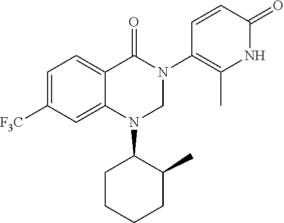 [SMILES: Cc1[nH]c(=O)ccc1N1CN([C@@H]2CCCC[C@@H]2C)c2cc(C(F)(F)F)ccc2C1=O] | ¹H NMR (400 MHz, CHLOROFORM-d) δ: 8.15 (d, J = 8.3 Hz, 1H), 7.39-7.28 (m, 1H), 7.14-7.10 (m, 1H), 7.05 (s, 1H), 6.53-6.47 (m, 1H), 4.96-4.66 (m, 2H), 3.84-3.74 (m, 1H), 2.47-2.38 (m, 1H), 2.34 (br d, J = 12.7 Hz, 3H), 1.98-1.80 (m, 2H), 1.75-1.61 (m, 3H), 1.55-1.40 (m, 3H), 1.01 (d, J = 6.8 Hz, | 3-(6-methoxy-2- methylpyridin-3- yl)-1-((1R,2S)-2- methylcyclohexyl)- 7-(trifluoromethyl)- 2,3- dihydroquinazolin- 4(1H)-one |
| | | | 3H). | |
| | | | MS (m/z) 430.4 (M + H)⁺ | |
| 163 | N-(3-(1-(4-fluoro- 2-methylphenyl)- 4-oxo-6- (trifluoromethyl)- 1,4- dihydroquinazolin- 3(2H)-yl)-6- oxo-1,6- dihydropyridin-2- yl)acetamide | 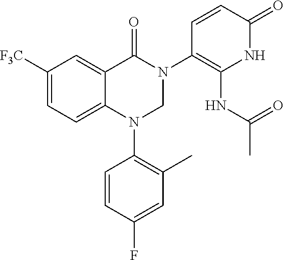 [SMILES: CC(=O)Nc1[nH]c(=O)ccc1N1CN(c2ccc(F)cc2C)c2ccc(C(F)(F)F)cc2C1=O] | ¹H NMR (CHLOROFORM-d, 400 MHz) δ 9.13 (br s, 1H), 8.17 (d, 1H, J = 1.5 Hz), 7.50 (dd, 1H, J = 2.2, 9.0 Hz), 7.2-7.3 (m, 2H), 7.19 (s, 1H), 7.15 (dd, 1H, J = 2.9, 8.8 Hz), 7.07 (dt, 1H, J = 2.9, 8.1 Hz), 6.39 (d, 1H, J = 8.8 Hz), 6.25 (d, 1H, J = 9.8 Hz), 5.20 (br d, 1H, J = 9.8 Hz), 4.86 (br d, 1H, J = 9.8 Hz), 2.35 (s, 3H), 2.21 (s, 3H) MS (m/z) 475 (M + H)⁺ | N-(3-(1-(4- fluoro-2- methylphenyl)- 4-oxo-6- (trifluoromethyl)- 1,4- dihydroquinazolin- 3(2H)-yl)-6- methoxypyridin- 2-yl)acetamide |
| 164 | 3-(2-bromo-6- oxo-1,6- dihydropyridin-3- yl)-1-(4-fluoro-2- methylphenyl)-6- (trifluoromethyl)- 2,3- dihydroquinazolin- 4(1H)-one | 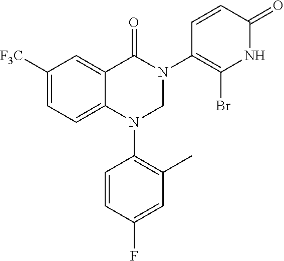 [SMILES: Cc1cc(F)ccc1N1CN(c2ccc(=O)[nH]c2Br)C(=O)c2cc(C(F)(F)F)ccc21] | ¹H NMR (CHLOROFORM-d, 400 MHz) δ 8.39 (d, 1H, J = 2.0 Hz), 7.65 (br d, 1H, J = 8.8 Hz), 7.53 (dd, 1H, J = 2.2, 8.6 Hz), 7.2- 7.3 (m, 1H), 7.1-7.2 (m, 2H), 7.0-7.1 (m, 1H), 6.82 (d, 1H, J = 8.3 Hz), 6.3-6.4 (m, 1H), 5.3-5.4 (m, 1H), 4.9-5.0 (m, 1H), 2.3-2.4 (m, 3H) MS (m/z) 496 (M + H)⁺/ 498 (M + 3H)⁺ | 3-(2-bromo-6- methoxypyridin- 3-yl)-1-(4- fluoro-2- methylphenyl)- 6-(trifluoro- methyl)-2,3- dihydroquinazolin- 4(1H)-one |
| 165 | 6-fluoro-1-(4- fluoro-2- methylphenyl)-7- methoxy-3-(2- methyl-6-oxo- 1,6- dihydropyridin-3- yl)-2,3- dihydropyrido[2, 3-d]pyrimidin- 4(1H)-one | 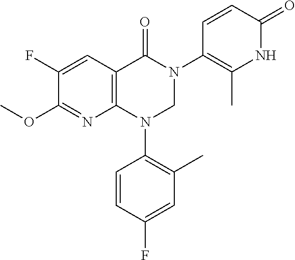 [SMILES: COc1nc2c(cc1F)C(=O)N(c1ccc(=O)[nH]c1C)CN2c1ccc(F)cc1C] | ¹H NMR (CHLOROFORM-d, 600 MHz) δ: 12.81 (br s, 1H), 7.93 (d, J = 9.8 Hz, 1H), 7.37 (br d, J = 9.6 Hz, 1H), 7.08-7.16 (m, 1H), 7.04 (dd, J = 9.1, 2.9 Hz, 1H), 6.96 (t, J = 8.1 Hz, 1H), 6.49 (d, J = 9.4 Hz, 1H), 4.56-5.51 (m, 2H), 3.70 (s, 3H), 2.35 (s, 3H), 2.27 (s, 3H) MS (m/z) 413 (M + H)⁺ | 6-fluoro-1-(4- fluoro-2- methylphenyl)- 7-methoxy-3-(6- methoxy-2- methylpyridin-3- yl)-2,3- dihydropyrido[2, 3-d]pyrimidin- 4(1H)-one |
| 166 | 1-(4-fluoro-2- methylphenyl)-3- (2-methyl-6-oxo- 1,6- dihydropyridin-3- yl)-4-oxo-7- (trifluoromethyl)- 1,2,3,4-tetra- hydroquinazoline- 6-carbonitrile | 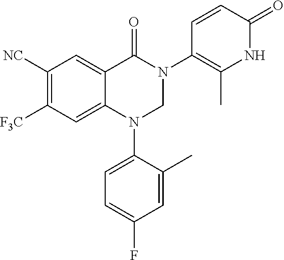 [SMILES: Cc1cc(F)ccc1N1CN(c2ccc(=O)[nH]c2C)C(=O)c2cc(C#N)c(C(F)(F)F)cc21] | ¹H NMR (400 MHz, DMSO-d₆) δ: 11.71- 12.03 (m, 1H) 8.34- 8.46 (m, 1H) 7.46- 7.55 (m, 1H) 7.34- 7.44 (m, 2 H) 7.21- 7.30 (m, 1H) 6.41- 6.53 (m, 1H) 6.22 (dd, J = 9.78, 2.93 Hz, 1H) 5.56 (d, J = 9.78 Hz, 0.6 H) 5.16-5.33 (m, 1H) 4.90-4.99 (m, 0.6H) 2.25 (s, 3H) 2.12 (d, J = 3.91 Hz, 3H) MS (m/z) 457.3 (M + H)⁺ | 1-(4-fluoro-2- methylphenyl)- 3-(6-methoxy-2- methylpyridin-3- yl)-4-oxo-7- (trifluoromethyl)- 1,2,3,4-tetra- hydroquinazoline- 6-carbonitrile |
| 167 | 6-chloro-7- (difluoromethoxy)- 1-(4-fluoro-2- methylphenyl)-3- (2-methyl-6-oxo- 1,6- dihydropyridin-3- yl)-2,3- dihydroquinazolin- 4(1H)-one | 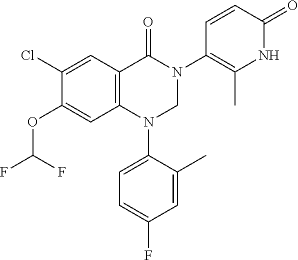 [SMILES: Cc1cc(F)ccc1N1CN(c2ccc(=O)[nH]c2C)C(=O)c2cc(Cl)c(OC(F)F)cc21] | ¹H NMR (400 MHz, DMSO-d₆) δ: 11.76 (br s, 1H), 7.92 (s, 1H), 7.36 (d, J = 9.8 Hz, 2H), 7.32 (dd, J = 9.78, 2.93 Hz, 1H), 7.22 (t, J = 73.36 Hz, 1H), 7.13- 7.21 (m, 1H), 6.20 (d, J- 9.78 Hz, 1H), 6.16- 6.01 (m, 1H), 5.57- 4.74 (m, 2H), 2.24 (s, 3H), 2.10 (br s, 3H). MS (m/z) 464.3 (M + H)⁺ | 6-chloro-7- (difluoromethoxy)- 1-(4-fluoro-2- methylphenyl)- 3-(6-methoxy-2- methylpyridin-3- yl)-2,3- dihydroquinazolin- 4(1H)-one |
| 168 | 6-chloro-7- (difluoromethyl)- 1-(4-fluoro-2- methylphenyl)-3- (2-methyl-6-oxo- 1,6- dihydropyridin-3- yl)-2,3- dihydroquinazolin- 4(1H)-one | 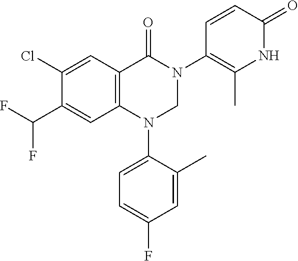 [SMILES: Cc1cc(F)ccc1N1CN(c2ccc(=O)[nH]c2C)C(=O)c2cc(Cl)c(C(F)F)cc21] | ¹H NMR (DMSO-d6, 400 MHz) δ: 11.78 (br s, 1H), 8.0-7.9 (m, 1H), 7.37 (d, J = 9.8 Hz, 2H), 7.32 (dd, J = 2.7, 9.5 Hz, 1H), 7.19-6.90 (m, 2H), 6.60-6.30 (m, 1H), 6.20 (d, J = 9.3 Hz, 1H), 5.50- 4.80 (m, 2H), 2.23 (s, 3H), 2.20-2.00 (m, 3H). MS (m/z) 447.0/449.0 (M + H)⁺ | 6-chloro-7- (difluoromethyl)-1- (4-fluoro-2- methylphenyl)- 3-(6-methoxy-2- methylpyridin-3- yl)-2,3- dihydro- quinazolin- 4(1H)-one |
| 169 | 3-(4-amino-2- oxo-1,2- dihydropyrimidin- 5-yl)-1-(4-fluoro- 2-methylphenyl)-6- (trifluoromethyl)- 2,3- dihydroquinazolin- 4(1H)-one | 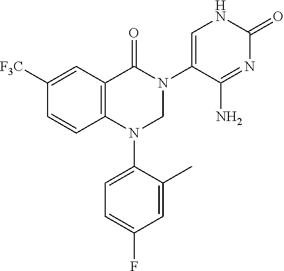 [SMILES: Cc1cc(F)ccc1N1CN(c2c[nH]c(=O)nc2N)C(=O)c2cc(C(F)(F)F)ccc21] | ¹H NMR (400 MHz, DMSO-d₆) δ: 10.45- 11.35 (m, 2H) 8.02- 8.12 (m, 1H) 7.67 (s, 1 H) 7.62 (dd, J = 8.80, 1.96 Hz, 1H) 7.36- 7.55 (m, 2H) 7.31 (dd, J = 9.54, 2.69 Hz, 1H) 7.15-7.23 (m, 1H) 6.30 (d, J = 8.80 Hz, 1H) 4.89-5.39 (m, 2H) 2.15-2.33 (m, 3H). MS (m/z) 434.3 (M + H)⁺ | 3-(4-amino-2- methoxypyrimidin- 5-yl)-1-(4- fluoro-2- methylphenyl)-6- (trifluoromethyl)-2,3- dihydroquinazolin- 4(1H)-one |
| 170 | 6-chloro-1-(2- methyl-4- (trifluoromethoxy) phenyl)-3-(2- methyl-6-oxo- 1,6- dihydropyridin-3- yl)-4-oxo- 1,2,3,4-tetra- hydropyrido[2,3- d]pyrimidine- 7-carbonitrile | 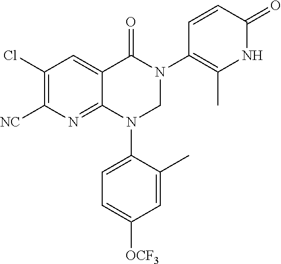 [SMILES: Cc1cc(OC(F)(F)F)ccc1N1CN(c2ccc(=O)[nH]c2C)C(=O)c2cc(Cl)c(C#N)nc21] | ¹H NMR (400 MHz, DMSO-d₆) δ 11.84 (br s, 1H), 8.39 (s, 1H), 7.51- 7.36 (m, 3H), 7.36- 7.31 (m, 1H), 6.21 (br d, J = 10.3 Hz, 1H), 5.64- 5.57 (m, 0.6H), 5.38- 5.32 (m, 0.4H), 5.18 (dt, J = 5.1, 2.3 Hz, 0.4H), 4.95 (br d, J = 9.3 Hz, 0.6H), 2.24 (s, 3H), 2.12 (s, 3H). MS (m/z) 490.2 (M + H)⁺ | 6-chloro-3-(6- methoxy-2- methylpyridin-3- yl)-1-(2-methyl-4- (trifluoromethoxy) phenyl)-4- oxo-1,2,3,4-tetra- hydropyrido[2,3- d]pyrimidine-7- carbonitrile |
| 171 | 6-chloro-1-(4- fluoro-2- methylphenyl)-3- (2-methyl-6-oxo- 1,6- dihydropyridin-3- yl)-4-oxo- 1,2,3,4-tetra- hydropyrido[2,3- d]pyrimidine- 7-carbonitrile | 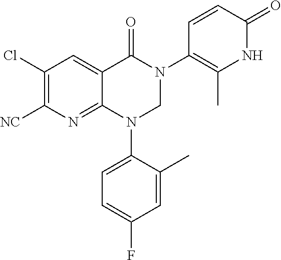 [SMILES: Cc1cc(F)ccc1N1CN(c2ccc(=O)[nH]c2C)C(=O)c2cc(Cl)c(C#N)nc21] | ¹H NMR (400 MHz, DMSO-d₆) δ 11.92- 11.78 (m, 1H), 8.36 (s, 1H), 7.47-7.32 (m, 2H), 7.27 (dd, J = 9.5, 2.7 Hz, 1H), 7.16 (td, J = 8.6, 2.9 Hz, 1H), 6.22 (br d, J = 7.3 Hz, 1H), 5.56 (br d, J = 8.8 Hz, 0.6H), 5.29 (br d, J = 10.3 Hz, 0.4H), 5.17 (br s, 0.4H),4.90 (br d, J = 9.3 Hz, 0.6H), 2.21 (s, 3H), 2.16-2.07 (m, 3H). MS (m/z) 424.2 (M + H)⁺ | 6-chloro-1-(4- fluoro-2- methylphenyl)- 3-(6-methoxy-2- methylpyridin-3- yl)-4-oxo- 1,2,3,4-tetra- hydropyrido[2,3- d]pyrimidine-7- carbonitrile |
| 172 | 6-chloro-1-(2- methyl-4- (trifluoromethoxy) phenyl)-3-(2- methyl-6-oxo- 1,6- dihydropyridin-3- yl)-4-oxo- 1,2,3,4-tetra- hydroquinazoline- 7-carbonitrile | 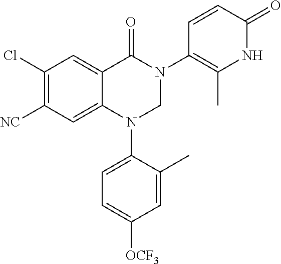 [SMILES: Cc1cc(OC(F)(F)F)ccc1N1CN(c2ccc(=O)[nH]c2C)C(=O)c2cc(Cl)c(C#N)cc21] | ¹H NMR (400 MHz, DMSO-d₆) δ 11.79 (br s, 1H), 8.01 (s, 1H), 7.51- 7.40 (m, 2H), 7.40- 7.27 (m, 2H), 7.02- 6.72 (m, 1H), 6.19 (d, J = 9.8 Hz, 1H), 5.55- 5.39 (m, 0.6H), 5.26- 5.06 (m, 0.8H), 4.97- 4.78 (m, 0.6H), 2.27 (s, 3H), 2.08 (br s, 3H). MS (m/z) 489.1 (M + H)⁺ | 6-chloro-3-(6- methoxy-2- methylpyridin-3- yl)-1-(2-methyl-4- (trifluoromethoxy) phenyl)-4- oxo-1,2,3,4- tetra- hydroquinazoline- 7-carbonitrile |
| 173 | 6-chloro-1-(4- fluoro-2- methylphenyl)-3- (6-methyl-2-oxo- 1,2- dihydropyrimidin- 5-yl)-4-oxo- 1,2,3,4-tetra- hydroquinazoline- 7-carbonitrile | 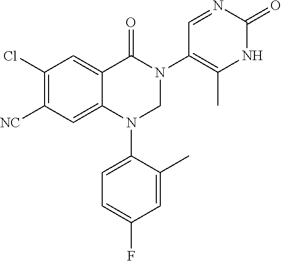 [SMILES: Cc1cc(F)ccc1N1CN(c2cnc(=O)[nH]c2C)C(=O)c2cc(Cl)c(C#N)cc21] | ¹H NMR (400 MHz, DMSO-d₆) δ 12.12 (br s, 1H), 8.02 (s, 1H), 7.41- 7.26 (m, 2H), 7.24- 7.16 (m, 1H), 6.87 (br s, 1H), 6.75 (br s, 1H), 5.51-5.43 (m, 0.6H), 5.25-5.14 (m, 0.8H), 4.93 (dt, J = 7.9, 4.1 Hz, 0.6H), 2.24 (br s, 3H), 2.21-2.08 (m, 3H). MS (m/z) 424.1 (M + H)⁺ | 6-chloro-1-(4- fluoro-2- methylphenyl)- 3-(2-methoxy-4- methylpyrimidin- 5-yl)-4-oxo- 1,2,3,4-tetra- hydroquinazoline- 7-carbonitrile |
| 174 | 1-(2-methyl-3- (trifluoromethyl) phenyl)-3-(2- methyl-6-oxo-1,6- dihydropyridin-3- yl)-6- (trifluoromethyl)- 2,3- dihydropyrido[2, 3-d]pyrimidin- 4(1H)-one | 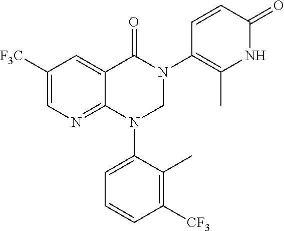 [SMILES: Cc1[nH]c(=O)ccc1N1CN(c2cccc(C(F)(F)F)c2C)c2ncc(C(F)(F)F)cc2C1=O] | ¹H NMR (400 MHz, DMSO-d₆) δ 11.83 (br s, 1H), 8.61 (s, 1H), 8.33 (d, J = 2.4 Hz, 1H), 7.75 (d, J = 7.8 Hz, 1H), 7.73- 7.65 (m, 1H), 7.57- 7.50 (m, 1H), 7.42 (dd, J = 9.3, 3.9 Hz, 1H), 6.27- 6.17 (m, 1H), 5.66 (d, J = 9.8 Hz, 0.6H), 5.43 (d, J = 10.3 Hz, 0.4H), 5.21 (d, J = 9.8 Hz, 0.4H), 5.00 (d, J = 9.3 Hz, 0.6H), 2.33 (s, 3H), 2.14 (d, J = 3.9 Hz, 3H). MS (m/z) 483.3 (M + H)⁺ | 3-(6-methoxy-2- methylpyridin-3- yl)-1-(2-methyl-3- (trifluoromethyl) phenyl)-6- (trifluoromethyl)-2,3- dihydropyrido[2,3- d]pyrimidin- 4(1H)-one |
| 175 | 1-(3-chloro-2- methylphenyl)-3- (2-methyl-6-oxo- 1,6- dihydropyridin-3- yl)-6- (trifluoromethyl)- 2,3- dihydropyrido[2,3- d]pyrimidin- 4(1H)-one | 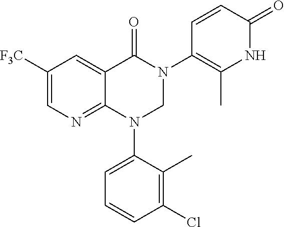 [SMILES: Cc1[nH]c(=O)ccc1N1CN(c2cccc(Cl)c2C)c2ncc(C(F)(F)F)cc2C1=O] | ¹H NMR (400 MHz, DMSO-d₆) δ 11.83 (br s, 1H), 8.60 (s, 1H), 8.32 (d, J = 2.4 Hz, 1H), 7.52- 7.48 (m, 1H), 7.45- 7.32 (m, 3H), 6.23 (br d, J = 8.8 Hz, 1H), 5.64 (d, J = 9.3 Hz, 0.6H), 5.38 (d, J = 9.8 Hz, 0.4H), 5.21 (d, J = 10.3 Hz, 0.4H), 4.97 (d, J = 9.3 Hz, 0.6H), 2.23 (s, 3H), 2.14 (d, J = 6.4 Hz, 3H). MS (m/z) 449.2 (M + H)⁺ | 1-(3-chloro-2- methylphenyl)- 3-(6-methoxy-2- methylpyridin-3- yl)-6- (trifluoromethyl)-2,3- dihydropyrido[2,3- d]pyrimidin- 4(1H)-one |
| 176 | 1-(2-ethyl-4- fluorophenyl)-3- (2-methyl-6-oxo- 1,6- dihydropyridin-3- yl)-6- (trifluoromethyl)- 2,3- dihydropyrido[2,3- d]pyrimidin- 4(1H)-one | 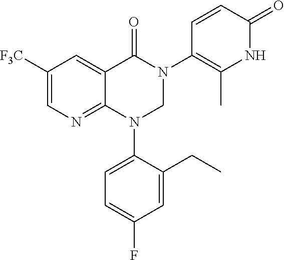 [SMILES: CCc1cc(F)ccc1N1CN(c2ccc(=O)[nH]c2C)C(=O)c2cc(C(F)(F)F)cnc21] | ¹H NMR (400 MHz, DMSO-d₆) δ 11.90- 11.75 (m, 1H), 8.58 (d, J = 1.0 Hz, 1H), 8.30 (d, J = 2.4 Hz, 1H), 7.48- 7.33 (m, 2H), 7.30- 7.07 (m, 2H), 6.22 (br dd, J = 9.3, 5.9 Hz, 1H), 5.63 (d, J = 9.3 Hz, 0.6H), 5.38 (d, J = 9.8 Hz, 0.4H), 5.09 (d, J = 10.3 Hz, 0.4H), 4.87 (d, J = 9.3 Hz, 0.6H), 2.56 (ddd, J = 15.4, 7.8, 3.2 Hz, 2H), 2.14 (d, J = 11.2 Hz, 3H), 1.14 (td, J = 7.6, 5.4 Hz, 3H). MS (m/z) 447.3 (M + H)⁺ | 1-(2-ethyl-4- fluorophenyl)-3- (6-methoxy-2- methylpyridin-3- yl)-6- (trifluoromethyl)-2,3- dihydropyrido[2,3- d]pyrimidin- 4(1H)-one |
| 177 | 1-(3,4-difluoro-2- methylphenyl)-3- (2-methyl-6-oxo- 1,6- dihydropyridin-3- yl)-6- (trifluoromethyl)- 2,3- dihydropyrido[2,3- d]pyrimidin- 4(1H)-one | 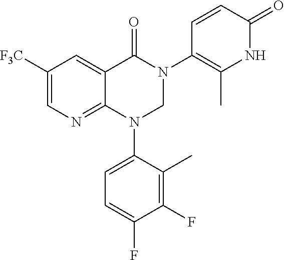 [SMILES: Cc1[nH]c(=O)ccc1N1CN(c2ccc(F)c(F)c2C)c2ncc(C(F)(F)F)cc2C1=O] | ¹H NMR (400 MHz, DMSO-d₆) δ 11.84 (br s, 1H), 8.67-8.56 (m, 1H), 8.32 (d, J = 2.4 Hz, 1H), 7.46-7.34 (m, 2H), 7.32-7.18 (m, 1H), 6.29-6.11 (m, 1H), 5.61 (br d, J = 9.8 Hz, 0.6H), 5.36 (br d, J = 9.3 Hz, 0.4H), 5.19 (br d, J = 9.8 Hz, 0.41H), 4.95 (d, J = 9.8 Hz, 0.6H), 2.21-2.06 (m, 6H). MS (m/z) 451.3 (M + H)⁺ | 1-(3,4-difluoro-2- methylphenyl)- 3-(6-methoxy-2- methylpyridin-3- yl)-6- (trifluoromethyl)-2,3- dihydropyrido[2,3- d]pyrimidin- 4(1H)-one |
| 178 | 1-(4-fluoro-2- isopropylphenyl)- 3-(2-methyl-6- oxo-1,6- dihydropyridin-3- yl)-6- (trifluoromethyl)- 2,3- dihydroquinazolin- 4(1H)-one | 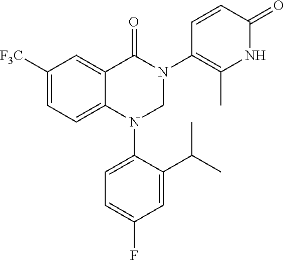 [SMILES: Cc1[nH]c(=O)ccc1N1CN(c2ccc(F)cc2C(C)C)c2ccc(C(F)(F)F)cc2C1=O] | ¹H NMR (400 MHz, DMSO-d₆) δ 11.80 (br s, 1H), 8.12-8.03 (m, 1H), 7.65 (dd, J = 9.0, 2.2 Hz, 1H), 7.47-7.28 (m, 3H), 7.27-7.16 (m, 1H), 6.39-6.25 (m, 1H), 6.25-6.16 (m, 1H), 5.56 (d, J = 9.3 Hz, 0.6H), 5.28 (d, J = 10.3 Hz, 0.4H), 4.98 (d, J = 10.3 Hz, 0.4H), 4.71 (d, J = 9.8 Hz 0.6H), 3.23- 3.02 (m, 1H), 2.14 (s, 3H), 1.23-1.10 (m, 6H). MS (m/z) 460.2 (M + H)⁺ | 1-(4-fluoro-2- isopropylphenyl)- 3-(6-methoxy- 2-methylpyridin- 3-yl)-6- (trifluoromethyl)-2,3- dihydroquinazolin- 4(1H)-one |
| 179 | 1-(4-fluoro-2- methylphenyl)-3- (4-methyl-6-oxo- 1,6- dihydropyridin-3- yl)-6- (trifluoromethyl)- 2,3- dihydroquinazolin- 4(1H)-one | 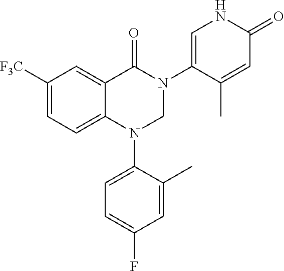 [SMILES: Cc1cc(=O)[nH]cc1N1CN(c2ccc(F)cc2C)c2ccc(C(F)(F)F)cc2C1=O] | ¹H NMR (400 MHz, DMSO-d₆) δ 11.56 (br s, 1H), 8.14-8.05 (m, 1H), 7.65 (dd, J = 8.8, 2.0 Hz, 1H), 7.51 (s, 1H), 7.39 (dd, J = 8.8, 5.4 Hz, 1H), 7.32 (dd, J = 9.5, 2.7 Hz, 1H), 7.19 (br d, J = 5.9 Hz, 1H), 6.42-6.29 (m, 1H), 6.26 (s, 1H), 5.54 (br d, J = 9.8 Hz, 0.6H), 5.24- 5.09 (m, 0.8H), 4.81 (br d, J = 9.3 Hz, 0.6H), 2.30-2.19 (m, 3H), 2.05 (s, 3H). MS (m/z) 432.3 (M + H)⁺ | 1-(4-fluoro-2- methylphenyl)- 3-(6-methoxy-4- methylpyridin-3- yl)-6- (trifluoromethyl)-2,3- dihydroquinazolin- 4(1H)-one |
| 180 | 6-fluoro-1-(4- fluoro-2- isopropylphenyl)- 3-(2-methyl-6- oxo-1,6- dihydropyridin-3- yl)-2,3- dihydroquinazolin- 4(1H)-one | 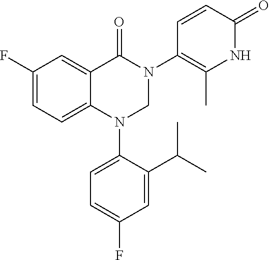 [SMILES: Cc1[nH]c(=O)ccc1N1CN(c2ccc(F)cc2C(C)C)c2ccc(F)cc2C1=O] | ¹H NMR (400 MHz, DMSO-d₆) δ: 11.81 (br. s., 1H), 7.56 (dd, J = 8.80, 2.45 Hz, 1H), 7.36-7.11 (m, 5H), 6.28-6.15 (m, 2H), 5.41- 4.62 (m, 2 H), 3.25- 3.10 (m, 1H), 2.12 (s, 2 H), 2.06 (s, 1H), 1.19- 1.11 (m, 6H). MS (m/z) 410.4 (M + H)⁺ | 6-fluoro-1-(4- fluoro-2- isopropylphenyl)- 3-(6-methoxy- 2-methylpyridin- 3-yl)-2,3- dihydroquinazolin- 4(1H)-one |
| 181 | 1-(2-cyclopropyl- 4-fluorophenyl)- 3-(2-methyl-6- oxo-1,6- dihydropyridin-3- yl)-2,3- dihydroquinazolin- 4(1H)-one | 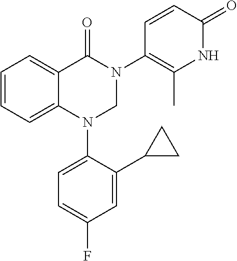 [SMILES: Cc1[nH]c(=O)ccc1N1CN(c2ccc(F)cc2C2CC2)c2ccccc2C1=O] | ¹H NMR (400 MHz, DMSO-d₆) δ: 11.75 (br. s., 1H), 7.85 (d, J = 7.58 Hz, 1H), 7.37-7.28 (m, 3H), 7.09 (br. s., 1H), 6.94-6.85 (m, 2H), 6.35-6.17 (m, 2H), 5.43-4.69 (m, 2H), 2.12 (br. s., 2 ), 2.06 (br. s., 2H), 0.96-0.70 (m, 4H). MS (m/z) 390.2 (M + H)⁺ | 1-(2- cyclopropyl-4- fluorophenyl)-3- (6-methoxy-2- methylpyridin-3- yl)-2,3- dihydroquinazolin- 4(1H)-one |
| 182 | 1-(2-cyclopropyl- 4-fluorophenyl)- 3-(2-methyl-6- oxo-1,6- dihydropyridin-3- yl)-7- (trifluoromethyl)- 2,3- dihydroquinazolin- 4(1H)-one | 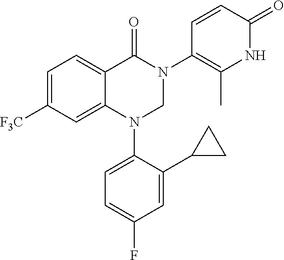 [SMILES: Cc1[nH]c(=O)ccc1N1CN(c2ccc(F)cc2C2CC2)c2cc(C(F)(F)F)ccc2C1=O] | ¹H NMR (400 MHz, DMSO-d₆) δ: 11.78 (br. s., 1H), 8.05 (d, J = 8.07 Hz, 1H), 7.42-7.37 (m, 2H), 7.22 (d, J = 8.07 Hz, 1H), 7.14 (t, J = 8.07 Hz, 1H), 6.92-6.88 (m, 1H), 6.43 (d, J = 12.0 Hz, 1H), 6.20 (d, J = 9.29 Hz, 1H), 5.56- 4.82 (m, 2H), 2.11 (s, 3 H), 1.98 (quin, J = 6.48 Hz, 1H), 0.95-0.67 (m, 4H). MS (m/z) 458.3 (M + H)⁺ | 1-(2- cyclopropyl-4- fluorophenyl)-3- (6-methoxy-2- methylpyridin-3- yl)-7- (trifluoromethyl)-2,3- dihydroquinazolin- 4(1H)-one |
| 183 | 5-fluoro-1-(4- fluoro-2- methylphenyl)-3- (2-methyl-6-oxo- 1,6- dihydropyridin-3- yl)-2,3- dihydroquinazolin- 4(1H)-one | 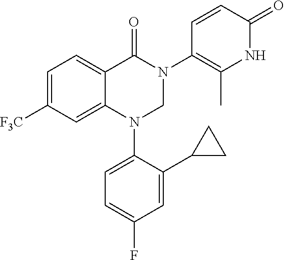 | ¹H NMR (400 MHz, DMSO-d₆) δ: 11.76 (br. s., 1H) 7.35-7.26 (m, 4 H) 7.16-7.12 (m, 1H) 6.71 (dd, J = 11.00, 8.31 Hz, 1H) 6.17 (d, J = 9.54 Hz, 1H) 6.04 (br. s., 1 H) 5.33-4.73 (m, 2H) 2.21 (s, 3H) 2.08 (br. s., 3H). MS (m/z) 382.3 (M + H)⁺ | 5-fluoro-1-(4- fluoro-2- methylphenyl)- 3-(6-methoxy-2- methylpyridin-3- yl)-2,3- dihydroquinazolin- 4(1H)-one |
| 184 | 1-(2-ethyl-4- fluorophenyl)-3- (2-methyl-6-oxo- 1,6- dihydropyridin-3- yl)-7- (trifluoromethyl)- 2,3- dihydroquinazolin- 4(1H)-one | 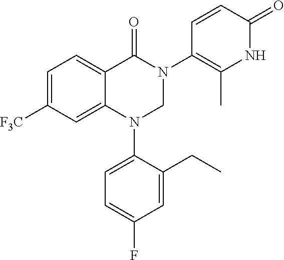 [SMILES: CCc1cc(F)ccc1N1CN(c2ccc(=O)[nH]c2C)C(=O)c2ccc(C(F)(F)F)cc21] | ¹H NMR (400 MHz, DMSO-d₆) δ: 11.79 (br. s., 1H), 8.06 (d, J = 8.07 Hz, 1H), 7.42-7.32 (m, 3 H), 7.22 (d, J = 7.82 Hz, 2 H), 6.36-6.18 (m, 2H), 5.54-4.71 (m, 2H), 2.61-2.55 (m, 2H), 2.11-2.09 (m, 3H), 1.13 (q, J = 7.58 Hz, 3H). MS (m/z) 446.3 (M + H)⁺ | 1-(2-ethyl-4- fluorophenyl)-3- (6-methoxy-2- methylpyridin-3- yl)-7- (trifluoromethyl)-2,3- dihydroquinazolin- 4(1H)-one |
| 185 | 7- (difluoromethoxy)- 1-(4-fluoro-2- methylphenyl)-3- (2-methyl-6-oxo- 1,6- dihydropyridin-3- yl)-2,3- dihydroquinazolin- 4(1H)-one | 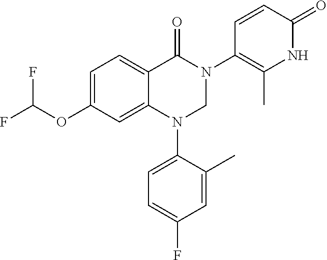 [SMILES: Cc1cc(F)ccc1N1CN(c2ccc(=O)[nH]c2C)C(=O)c2ccc(OC(F)F)cc21] | ¹H NMR (400 MHz, DMSO-d₆) δ: 11.76 (br. s., 1H), 7.90 (d, J = 8.56 Hz, 1H), 7.40-7.04 (m, 5H), 6.71 (dd, J = 8.56, 1.96 Hz, 1H), 6.18 (d, J = 9.54 Hz, 1H), 5.81 (br. s., 1H), 5.44-4.69 (m, 2H), 2.23 (s, 3H) 2.09 (br. s., 3H). MS (m/z) 430.3 (M + H)⁺ | 7- (difluoromethoxy)- 1-(4-fluoro-2- methylphenyl)- 3-(6-methoxy-2- methylpyridin-3- yl)-2,3- dihydroquinazolin- 4(1H)-one |
| 186 | 1-(4-fluoro-2- methylphenyl)-3- (6-oxo-1,6- dihydropyridin-3- yl)-7- (trifluoromethyl) quinazoline- 2,4(1H,3H)- dione | 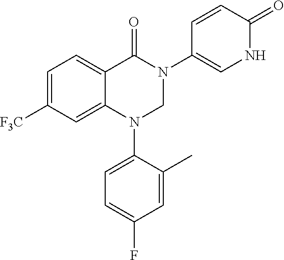 | ¹H NMR (400 MHz, DMSO-d₆) δ: 11.79 (br. s., 1H) 8.35 (d, J = 8.28 Hz, 1H) 7.69-7.67 (m, 1H) 7.56 (d, J = 2.51 Hz, 1H) 7.47-7.39 (m, 3H) 7.30 (td, J = 8.47, 2.89 Hz, 1H) 6.56 (s, 1H) 6.42 (d, J = 9.54 Hz, 1H) 2.12 (s, 3H). MS (m/z) 432.2 (M + H)⁺ | 1-(4-fluoro-2- methylphenyl)- 3-(6- methoxypyridin- 3-yl)-7- (trifluoromethyl) quinazoline- 2,4(1H,3H)- dione |
| 187 | 7-cyclopropyl-1- (4-fluoro-2- methylphenyl)-3- (2-methyl-6-oxo- 1,6- dihydropyridin-3- yl)-2,3- dihydroquinazolin- 4(1H)-one | 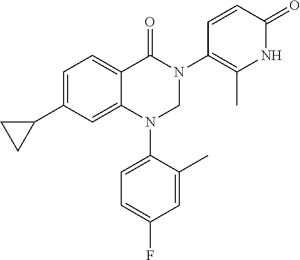 [SMILES: Cc1cc(F)ccc1N1CN(c2ccc(=O)[nH]c2C)C(=O)c2ccc(C3CC3)cc21] | ¹H NMR (400 MHz, DMSO-d₆) δ: 11.73 (br. s., 1H), 7.73 (d, J = 8.07 Hz, 1H), 7.33-7.25 (m, 3H), 7.15-7.12 (m, 1H), 6.56 (d, J = 8.07 Hz, 1H), 6.16 (d, J = 9.54 Hz, 1H), 5.96 (br. s., 1H), 5.33- 4.64 (m, 2H), 2.22 (s, 3H), 2.06 (br. s., 3H), 1.83-1.76 (m, 1H), 0.90 (dd, J = 8.31, 1.96 Hz, 2H), 0.56 (d, J = 3.18 Hz, 2H). MS (m/z) 404.3 (M + H)⁺ | 7-cyclopropyl-1- (4-fluoro-2- methylphenyl)- 3-(6-methoxy-2- methylpyridin-3- yl)-2,3- dihydroquinazolin- 4(1H)-one |
| 188 | 1-(4-fluoro-2- methylphenyl)-3- (2-methyl-6-oxo- 1,6- dihydropyridin-3- yl)-6- (trifluoromethoxy)- 2,3- dihydroquinazolin- 4(1H)-one | 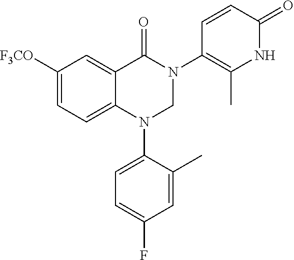 [SMILES: Cc1cc(F)ccc1N1CN(c2ccc(=O)[nH]c2C)C(=O)c2cc(OC(F)(F)F)ccc21] | ¹H NMR (400 MHz, DMSO-d₆) δ: 11.77 (br. s., 1H), 7.72 (d, J = 1.96 Hz, 1H), 7.38-7.28 (m, 4H), 7.16 (t, J = 6.60 Hz, 1H), 6.28 (br. s., 1H), 6.18 (d, J = 9.29 Hz, 1H), 5.43-4.73 (m, 2H), 2.23 (s, 3H), 2.10 (br. s., 3H). MS (m/z) 448.3 (M + H)⁺ | 1-(4-fluoro-2- methylphenyl)- 3-(6-methoxy-2- methylpyridin-3- yl)-6- (trifluoromethoxy)- 2,3- dihydroquinazolin- 4(1H)-one |
| 189 | 1-(4- (difluoromethoxy)-2- methylphenyl)-3- (2-methyl-6-oxo- 1,6- dihydropyridin-3- yl)-6- (trifluoromethyl)- 2,3- dihydroquinazolin- 4(1H)-one | 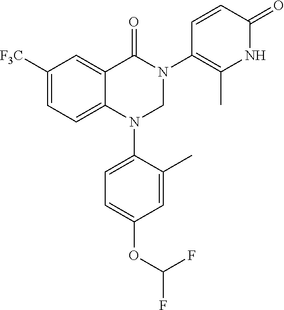 [SMILES: Cc1cc(OC(F)F)ccc1N1CN(c2ccc(=O)[nH]c2C)C(=O)c2cc(C(F)(F)F)ccc21] | ¹H NMR (400 MHz, DMSO-d₆) δ: 11.79 (br s, 1H), 8.07 (d, J = 2.0 Hz, 1H), 7.65 (dd, J = 2.4, 8.8 Hz, 1H), 7.48- 7.11 (m, 5H), 6.37-6.30 (m, 1H), 6.19 (d, J = 9.3 Hz, 1H), 5.52-4.77 (m, 2H), 2.23 (s, 3H), 2.11 (br s, 3H). MS (m/z) 480.3 (M + H)⁺ | 1-(4- (difluoromethoxy)- 2-methylphenyl)- 3-(6-methoxy-2- methylpyridin-3- yl)-6- (trifluoromethyl)-2,3- dihydroquinazolin- 4(1H)-one |
| 190 | 3-(2-ethyl-6-oxo- 1,6- dihydropyridin-3- yl)-1-(4-fluoro-2- methylphenyl)-7- (trifluoromethoxy)- 2,3- dihydroquinazolin- 4(1H)-one | 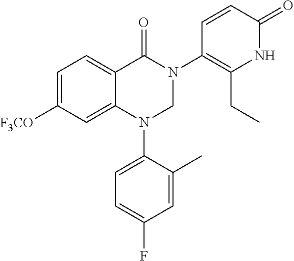 [SMILES: CCc1[nH]c(=O)ccc1N1CN(c2ccc(F)cc2C)c2cc(OC(F)(F)F)ccc2C1=O] | ¹H NMR (400 MHz, DMSO-d₆) δ: 11.74 (br s, 1H), 7.97 (d, J = 8.8 Hz, 1H), 7.39-7.29 (m, 3H), 7.19 (br, s, 1H), 6.88 (d, J = 8.8 Hz, 1H), 6.21 (d, J = 8.8 Hz, 1H), 6.07-5.96 (m, 1H), 5.57- 4.65 (m, 2H), 2.42 (br, s, 2H), 2.21 (s, 3H), 1.05 (t, J = 7.3 Hz, 3H). MS (m/z) 462.3 (M + H)⁺ | 3-(2-ethyl-6- methoxypyridin- 3-yl)-1-(4- fluoro-2- methylphenyl)-7- (trifluoromethoxy)- 2,3- dihydroquinazolin- 4(1H)-one |
| 191 | 6-chloro-1-(2- ethyl-4- fluorophenyl)-7- fluoro-3-(2- methyl-6-oxo- 1,6- dihydropyridin-3- yl)-2,3- dihydroquinazolin- 4(1H)-one | 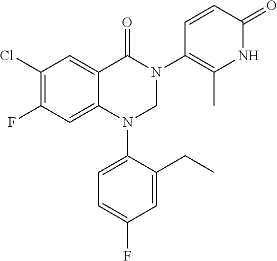 [SMILES: CCc1cc(F)ccc1N1CN(c2ccc(=O)[nH]c2C)C(=O)c2cc(Cl)c(F)cc21] | ¹H NMR (400 MHz, DMSO-d₆) δ: 11.79 (br s, 1H), 7.91 (d, J = 8.3 Hz, 1H), 7.40-7.30 (m, 3H), 7.22-7.18 (m, 1H), 6.20-6.04 (m, 2H), 5.49- 4.67 (m, 2H), 2.61-2.55 (m, 2H), 2.10-2.08 (m, 3H), 1.14 (q, J = 7.8 Hz, 3H). MS (m/z) 430.2 (M + H)⁺ | 6-chloro-1-(2- ethyl-4- fluorophenyl)-7- fluoro-3-(6- methoxy-2- methylpyridin-3- yl)-2,3- dihydroquinazolin- 4(1H)-one |
| 192 | 6-chloro-1-(2- ethyl-4- fluorophenyl)-3- (2-methyl-6-oxo- 1,6- dihydropyridin-3- yl)-4-oxo- 1,2,3,4-tetra- hydroquinazoline- 7-carbonitrile | 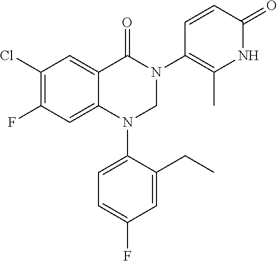 | ¹H NMR (400 MHz, DMSO-d₆) δ: 11.80 (br s, 1H), 7.99 (s, 1H), 7.4- 7.31 (m, 3H), 7.22-7.20 (m, 1H), 6.76-6.68 (m, 1H), 6.19 (d, J = 9.3 Hz, 1H), 5.50-4.74 (m, 2H), 2.58 (q, J = 7.3 Hz, 2H), 2.09 (br d, J = 12.2 Hz, 3H), 1.14 (q, J = 7.5 Hz, 3H). MS (m/z) 437.3 (M + H)⁺ | 6-chloro-1-(2- ethyl-4- fluorophenyl)-3- (6-methoxy-2- methylpyridin-3- yl)-4-oxo- 1,2,3,4-tetra- hydroquinazoline- 7-carbonitrile |
| 193 | 6-chloro-5,7- difluoro-1-(4- fluoro-2- methylphenyl)-3- (2-methyl-6-oxo- 1,6- dihydropyridin-3- yl)-2,3- dihydroquinazolin- 4(1H)-one | 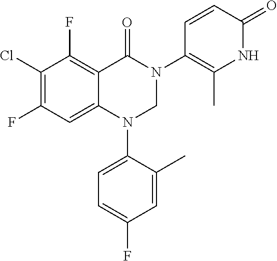 [SMILES: Cc1cc(F)ccc1N1CN(c2ccc(=O)[nH]c2C)C(=O)c2c1cc(F)c(Cl)c2F] | ¹H NMR (400 MHz, DMSO-d₆) δ: 11.77 (br s, 1H), 7.36-7.28 (m, 3H), 7.18 (br s, 1H), 6.18 (d, J = 9.8 Hz, 1H), 6.12-6.02 (m, 1H), 5.37- 4.79 (m, 2H), 2.22 (s, 3H), 2.08 (br d, J = 10.3 Hz, 3H). MS (m/z) 434.3 (M + H)⁺ | 6-chloro-5,7- difluoro-1-(4- fluoro-2- methylphenyl)- 3-(6-methoxy-2- methylpyridin-3- yl)-2,3- dihydroquinazolin- 4(1H)-one |
| 194 | 6-chloro-5- fluoro-1-(4- fluoro-2- methylphenyl)-3- (2-methyl-6-oxo- 1,6- dihydropyridin-3- yl)-4-oxo- 1,2,3,4-tetra- hydroquinazoline- 7-carbonitrile | 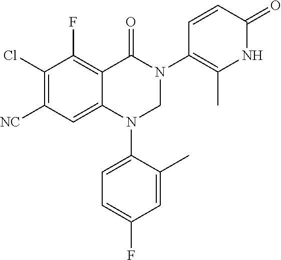 [SMILES: Cc1cc(F)ccc1N1CN(c2ccc(=O)[nH]c2C)C(=O)c2c1cc(C#N)c(Cl)c2F] | ¹H NMR (400 MHz, DMSO-d₆) δ: 11.78 (br s, 1H), 7.36-7.29 (m, 3H), 7.17 (br s, 1H), 6.72 (d, J = 1.0 Hz, 1H), 6.18 (d, J = 9.8 Hz, 1H), 5.6-4.87 (m, 2H), 2.22 (s, 3H), 2.08 (br d, J = 14.7 Hz, 3H). MS (m/z) 441.3 (M + H)⁺ | 6-chloro-5- fluoro-1-(4- fluoro-2- methylphenyl)- 3-(6-methoxy-2- methylpyridin-3- yl)-4-oxo- 1,2,3,4-tetra- hydroquinazoline- 7-carbonitrile |
| 195 | 1-(2-methyl-4- (trifluoromethyl) phenyl)-3-(2- methyl-6-oxo- 1,6- dihydropyridin-3- yl)-6- (trifluoromethyl)- 2,3- dihydropyrido[2,3- d]pyrimidin- 4(1H)-one | 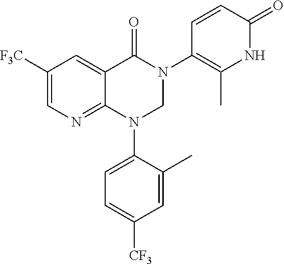 [SMILES: Cc1cc(C(F)(F)F)ccc1N1CN(c2ccc(=O)[nH]c2C)C(=O)c2cc(C(F)(F)F)cnc21] | ¹H NMR (400 MHz, DMSO-d₆) δ: 11.76 (br s, 1H), 8.60 (d, J = 2.0 Hz, 1H), 8.34 (d, J = 2.9 Hz, 1H), 7.78 (s, 1H), 7.69-7.67 (m, 1H), 7.56 (br d, J = 8.3 Hz, 1H), 7.42 (br d, J = 10.3 Hz, 1H), 6.22 (br d, J = 9.3 Hz, 1H), 5.71-5.00 (m, 2H), 2.27 (s, 3H), 2.13 (s, 3H). MS (m/z) 483.3 (M + H)⁺ | 3-(6-methoxy-2- methylpyridin-3- yl)-1-(2-methyl-4- (trifluoromethyl) phenyl)-6- (trifluoromethyl)-2,3- dihydropyrido[2,3- d]pyrimidin- 4(1H)-one |
| 196 | 1-(4-chloro-2- methylphenyl)-3- (2-methyl-6-oxo- 1,6- dihydropyridin-3- yl)-6- (trifluoromethyl)- 2,3- dihydropyrido[2,3- d]pyrimidin- 4(1H)-one | 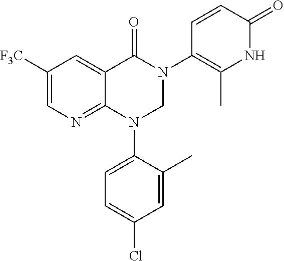 [SMILES: Cc1cc(Cl)ccc1N1CN(c2ccc(=O)[nH]c2C)C(=O)c2cc(C(F)(F)F)cnc21] | ¹H NMR (400 MHz, DMSO-d₆) δ: 11.82 (br s, 1H), 8.58 (d, J = 2.0 Hz, 1H), 8.31 (d, J = 2.9 Hz, 1H), 7.48-7.37 (m, 4H), 6.22 (br d, J = 8.8 Hz, 1H), 5.63-4.91 (m, 2H), 2.19 (s, 3H), 2.13 (br s, 3H). MS (m/z) 449.3 (M + H)⁺ | 1-(4-chloro-2- methylphenyl)- 3-(6-methoxy-2- methylpyridin-3- yl)-6- (trifluoromethyl)-2,3- dihydropyrido[2,3- d]pyrimidin- 4(1H)-one |
| 197 | 7-(1,1-difluoro-2- hydroxyethyl)-1- (4-fluoro-2- methylphenyl)-3- (2-methyl-6-oxo- 1,6- dihydropyridin-3- yl)-2,3- dihydroquinazolin- 4(1H)-one | 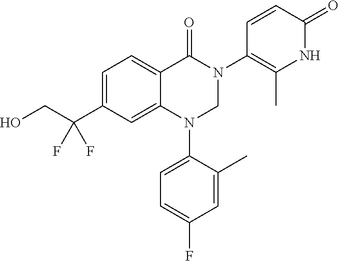 [SMILES: Cc1cc(F)ccc1N1CN(c2ccc(=O)[nH]c2C)C(=O)c2ccc(C(F)(F)CO)cc21] | ¹H NMR (400 MHz, DMSO-d₆) δ: 11.74 (br s, 1H), 7.96 (d, J = 8.07 Hz, 1H), 7.43-7.25 (m, 3H), 7.25-7.11 (m, 1H), 7.11-7.01 (m, 1H), 6.30 (br s, 1H), 6.19 (d, J = 9.54 Hz, 1H), 5.56 (t, J = 6.36 Hz, 1H), 5.44 (br s, 0.5H), 5.08 (br s, 1H), 4.74 (br s, 0.5H), 3.74 (td, J = 13.94, 6.36 Hz, 2H), 2.23 (s, 3H), 2.10 (s, 3H). MS (m/z) 444.2 (M + H)⁺ | 7-(1,1-difluoro- 2-hydroxyethyl)- 1-(4-fluoro-2- methylphenyl)- 3-(6-methoxy-2- methylpyridin-3- yl)-2,3- dihydroquinazolin- 4(1H)-one |
| 198 | 1-(4-fluoro-2- methylphenyl)-3- (2-isopropyl-6- oxo-1,6- dihydropyridin-3- yl)-7- (trifluoromethyl)- 2,3- dihydroquinazolin- 4(1H)-one | 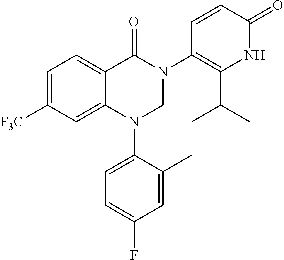 [SMILES: Cc1cc(F)ccc1N1CN(c2ccc(=O)[nH]c2C(C)C)C(=O)c2ccc(C(F)(F)F)cc21] | ¹H NMR (400 MHz, DMSO-d₆) δ: 11.55 (br s, 1H), 8.07 (d, J = 8.3 Hz, 1H), 7.47-7.29 (m, 3H), 7.28-7.06 (m, 2H), 6.59-6.12 (m, 2H), 5.55 (br d, J = 7.8 Hz, 0.6H), 5.23-4.96 (m, 0.8H), 4.68 (br d, J = 8.3 Hz, 0.6H), 3.18-2.87 (m, 1H), 2.30-2.15 (m, 3H), 1.24-0.97 (m, 6H) MS (m/z) 460.1 (M + H)⁺ | 1-(4-fluoro-2- methylphenyl)- 3-(2-isopropyl-6- methoxypyridin- 3-yl)-7- (trifluoromethyl)-2,3- dihydroquinazolin- 4(1H)-one |
| 199 | 1-(4-fluoro-2- methylphenyl)-3- (2-((2-hydroxy- ethyl)amino)- 6-oxo-1,6- dihydropyridin-3- yl)-7- (trifluoromethyl)- 2,3- dihydroquinazolin- 4(1H)-one | 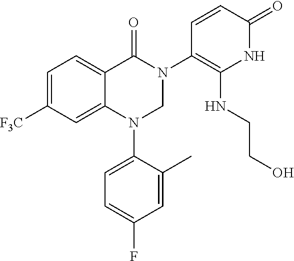 [SMILES: Cc1cc(F)ccc1N1CN(c2ccc(=O)[nH]c2NCCO)C(=O)c2ccc(C(F)(F)F)cc21] | ¹H NMR (400 MHz, DMSO-d₆) δ: 8.07 (d, J = 7.8 Hz, 1H), 7.61- 7.35 (m, 1H), 7.31 (br dd, J = 2.9, 9.8 Hz, 1H), 7.27 (d, J = 8.3 Hz, 1H), 7.25-7.20 (m, 1H), 7.20- 7.15 (m, 1H), 6.35 (s, 1H), 6.30-5.91 (m, 1H), 5.74 (br d, J = 7.8 Hz, 1H), 5.32 (br s, 0.6H), 5.04 (br s, 0.8H), 4.68 (br s, 0.6H), 3.47 (t, J = 4, 1H), 3.37-3.35 (m, 2H), 3.34-3.23 (m, 2H), 2.30-2.23 (m, 3H) MS (m/z) 477.0 (M + H)⁺ | 1-(4-fluoro-2- methylphenyl)- 3-(2-((2-hydroxy- ethyl)amino)-6- methoxypyridin- 3-yl)-7- (trifluoromethyl)-2,3- dihydroquinazolin- 4(1H)-one |
| 200 | 6-chloro-1-(2- (dimethylamino)- 4-fluorophenyl)- 3-(2-methyl-6- oxo-1,6- dihydropyridin-3- yl)-2,3- dihydroquinazolin- 4(1H)-one | 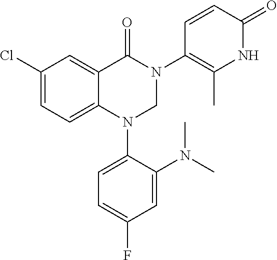 [SMILES: Cc1[nH]c(=O)ccc1N1CN(c2ccc(F)cc2N(C)C)c2ccc(Cl)cc2C1=O] | ¹H NMR (400 MHz, DMSO-d₆) δ: 11.75 (br s, 1H), 7.79 (d, J = 2.4 Hz, 1H), 7.40 (br dd, J = 2.2, 9.0 Hz, 1H), 7.31 (br d, J = 9.3 Hz, 1H), 7.23-7.13 (m, 1H), 6.92-6.86 (m, 1H), 6.81-6.75 (m, 1H), 6.67-6.35 (m, 1H), 6.19 (d, J = 9.3 Hz, 1H), 5.42-5.03 (m, 1H), 5.03-4.66 (m, 1H), 2.73 (s, 6H), 2.08 (s, 3H). MS (m/z) 427.2 (M + H)⁺ | 6-chloro-1-(2- (dimethylamino)-4- fluorophenyl)-3- (6-methoxy-2- methylpyridin-3- yl)-2,3- dihydroquinazolin- 4(1H)-one |
| 201 | 6-chloro-1-(4- fluoro-2- (methylamino) phenyl)-3-(2- methyl-6-oxo- 1,6- dihydropyridin-3- yl)-2,3- dihydroquinazolin- 4(1H)-one | 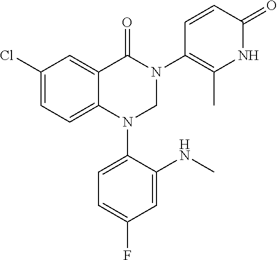 [SMILES: CNc1cc(F)ccc1N1CN(c2ccc(=O)[nH]c2C)C(=O)c2cc(Cl)ccc21] | ¹H NMR (400 MHz, DMSO-d₆) δ: 11.74 (br s, 1H), 7.77 (d, J = 2.9 Hz, 1H), 7.49 (d, J = 9.3 Hz, 0.5H), 7.37 (dd, J = 2.9, 8.8 Hz, 1H), 7.34 (d, J = 9.6 Hz, 0.5H), 7.17-7.06 (m, 1H), 6.45 (dd, J = 2.4, 11.7 Hz, 1H), 6.43-6.35 (m, 1H) 6.31-6.14 (m, 2H), 6.08 (br d, J = 3.9 Hz, 0.5H), 5.92 (br d, J = 3.4 Hz, 0.5H), 5.28 (d, J = 9.3 Hz, 0.5H), 5.14 (d, J = 9.8 Hz, 0.5H), 4.69 (d, J = 9.8 Hz, 0.5H), 4.56 (d, J = 9.3 Hz, 0.5H), 2.74-2.64 (m, 3H), 2.13 (d, 1.5H, J = 42.06 Hz, 3H). MS (m/z) 413.3 (M + H)⁺ | 6-chloro-1-(4- fluoro-2- (methylamino) phenyl)-3-(6- methoxy-2- methylpyridin-3- yl)-2,3- dihydroquinazolin- 4(1H)-one |
| 202 | 1-(4-(1,1- difluoro-2- hydroxyethyl)-2- methylphenyl)-3- (2-methyl-6-oxo- 1,6- dihydropyridin-3- yl)-7- (trifluoromethyl)- 2,3- dihydroquinazolin- 4(1H)-one | 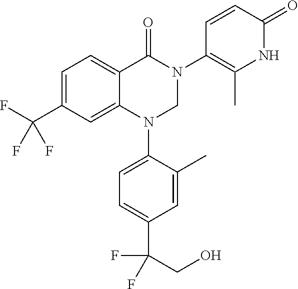 [SMILES: Cc1cc(C(F)(F)CO)ccc1N1CN(c2ccc(=O)[nH]c2C)C(=O)c2ccc(C(F)(F)F)cc21] | ¹H NMR (400 MHz, DMSO-d₆) δ: 11.79 (br s, 1H), 8.10 (d, J = 8.3 Hz, 1H), 7.62-7.58 (m, 1H), 7.50 (br d, J = 7.8 Hz, 1H), 7.43-7.36 (m, 2H), 7.32-7.27 (m, 1H), 6.44 (br s, 1H), 6.19 (d, J = 9.3 Hz, 1H), 5.83- 4.76 (m, 2H), 3.90 (t, J = 13.9 Hz, 2H), 2.27 (s, 3H), 2.08 (br s, 3H). MS (m/z) 494.3 (M + H)⁺ | 1-(4-(1,1- difluoro-2- hydroxyethyl)-2- methylphenyl)- 3-(6-methoxy-2- methylpyridin-3- yl)-7- (trifluoromethyl)-2,3- dihydroquinazolin- 4(1H)-one |
| 203 | 1-(2,4- dimethylphenyl)- 3-(2-methyl-6- oxo-1,6- dihydropyridin-3- yl)-7- (trifluoromethyl)- 2,3- dihydroquinazolin- 4(1H)-one | 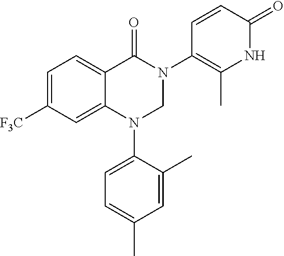 [SMILES: Cc1ccc(N2CN(c3ccc(=O)[nH]c3C)C(=O)c3ccc(C(F)(F)F)cc32)c(C)c1] | ¹H NMR (400 MHz, DMSO-d₆) δ: 11.79 (br s, 1H), 8.06 (d, J = 8.3 Hz, 1H), 7.39 (d, J = 9.8 Hz, 1H), 7.26 (s, 1H), 7.24-7.14 (m, 3H), 6.44-6.30 (m, 1H), 6.20 (br d, J = 9.8 Hz, 1H), 5.65-4.64 (m, 2H), 2.36-2.31 (m, 3H), 2.18 (s, 3H), 2.11 (br s, 3H). MS (m/z) 428.3 (M + H)⁺ | 1-(2,4- dimethylphenyl)- 3-(6-methoxy- 2-methylpyridin- 3-yl)-7- (trifluoromethyl)-2,3- dihydroquinazolin- 4(1H)-one |
| 204 | 1-(4-fluoro-2- methylphenyl)-3- (2-oxo-1,2- dihydropyridin-3- yl)-7- (trifluoromethyl)- 2,3- dihydroquinazolin- 4(1H)-one | 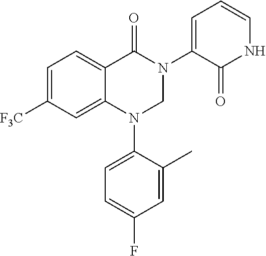 [SMILES: Cc1cc(F)ccc1N1CN(c2ccc[nH]c2=O)C(=O)c2ccc(C(F)(F)F)cc21] | ¹H NMR (400 MHz, DMSO-d₆) δ: 12.01 (br s, 1H), 8.08 (d, J = 8.3 Hz, 1H), 7.63 (dd, J = 7.3, 2.0 Hz, 1H), 7.46- 7.38 (m, 2H), 7.31 (dd, J = 9.8, 2.9 Hz, 1H), 7.28- 7.14 (m, 2H), 6.38- 6.35 (m, 1H), 6.27 (t, J = 6.8 Hz, 1H), 5.34 (br s, 1H), 4.91 (br s, 1H), 2.25 (s, 3H) MS (m/z) 418.2 (M + H)⁺ | 1-(4-fluoro-2- methylphenyl)- 3-(2- methoxypyridin- 3-yl)-7- (trifluoromethyl)-2,3- dihydroquinazolin- 4(1H)-one |
| 205 | 1-(4-fluoro-2- methylphenyl)-3- (6-methyl-2-oxo- 1,2- dihydropyrimidin- 5-yl)-7- (trifluoromethoxy)- 2,3- dihydroquinazolin- 4(1H)-one | 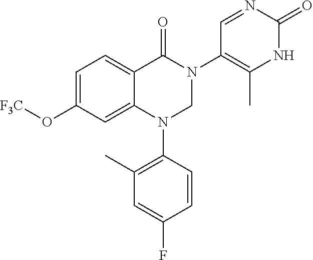 [SMILES: Cc1cc(F)ccc1N1CN(c2cnc(=O)[nH]c2C)C(=O)c2ccc(OC(F)(F)F)cc21] | ¹H NMR (400 MHz, DMSO-d₆) δ: 12.07 (br s, 1H), 8.42 (br s, 1H), 7.99 (d, J = 8.8 Hz, 1H), 7.42-7.35 (m, 1H), 7.32 (dd, J = 9.8, 2.9 Hz, 1H), 7.25-7.15 (m, 1H), 6.93- 6.86 (m, 1H), 6.12-5.95 (m, 1H), 5.60-4.75 (m, 2H), 2.24 (br s, 3H), 2.18 (br s, 3H). MS (m/z) 449.0 (M + H)⁺ | 1-(4-fluoro-2- methylphenyl)- 3-(2-methoxy-4- methylpyrimidin- 5-yl)-7- (trifluoromethoxy)- 2,3- dihydroquinazolin- 4(1H)-one |
| 206 | 1-(4-fluoro-2- methylphenyl)-7- methyl-3-(2- methyl-6-oxo-1,6- dihydropyridin-3- yl)-2,3- dihydropyrido[2,3- d]pyrimidin- 4(1H)-one | 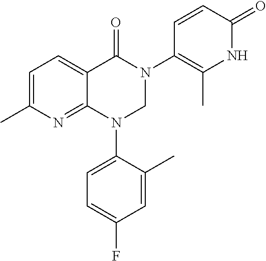 [SMILES: Cc1ccc2c(n1)N(c1ccc(F)cc1C)CN(c1ccc(=O)[nH]c1C)C2=O] | ¹H NMR (400 MHz, DMSO-d₆) δ: 11.77 (br s, 1H), 8.04 (d, J = 7.8 Hz, 1H), 7.38 (br d, J = 7.8 Hz, 1H), 7.28 (dd, J = 5.9, 8.8 Hz, 1H), 7.20 (dd, J = 2.9, 9.8 Hz, 1H), 7.09 (dt, J = 3.2, 8.4 Hz, 1H), 6.8-6.8 (m, J = 7.8 Hz, 1H), 6.19 (br d, J = 9.8 Hz, 1H), 5.3-5.6 (m, 1H), 4.7-4.9 (m, 1H), 2.26 (s, 3H), 2.18 (s, 3H), 2.10 (s, 3H). MS (m/z) 379.3 (M + H)⁺ | 1-(4-fluoro-2- methylphenyl)- 3-(6-methoxy-2- methylpyridin-3- yl)-7-methyl-2,3- dihydropyrido[2,3- d]pyrimidin- 4(1H)-one |
| 207 | 6-chloro-1-(2- ethyl-4- fluorophenyl)-3- (2-methyl-6-oxo-1,6- dihydropyridin-3- yl)-4-oxo-1,2,3,4- tetrahydropyrido [2,3-d]pyrimidine- 7-carbonitrile | 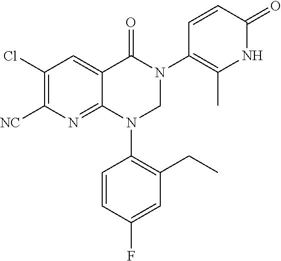 [SMILES: CCc1cc(F)ccc1N1CN(c2ccc(=O)[nH]c2C)C(=O)c2cc(Cl)c(C#N)nc21] | ¹H NMR (400 MHz, DMSO-d₆) δ: 11.85 (br s, 1H), 8.36 (s, 1H), 7.3- 7.5 (m, 2H), 7.24 (dd, 1H, J = 2.4, 9.8 Hz), 7.18 (dt, 1H, J = 2.9, 8.6 Hz), 6.22 (dd, 1H, J = 4.6, 9.6 Hz), 5.59 (d, 0.6H, J = 9.8 Hz), 5.34 (d, 0.4H, J = 10.3 Hz), 5.09 (d, 0.4H, J = 10.3 Hz), 4.86 (d, 0.6H, J = 9.8 Hz), 2.5-2.6 (m, 2H), 2.12 (d, 3H, J = 10.8 Hz), 0.9-1.2 (m, 3H) MS (m/z) 438.3 (M + H)⁺ | 6-chloro-1-(2- ethyl-4- fluorophenyl)-3- (6-methoxy-2- methylpyridin-3- yl)-4-oxo- 1,2,3,4-tetra- hydropyrido[2,3- d]pyrimidine-7- carbonitrile |
| 208 | 1-((1S,3S)-3- fluorocyclopentyl)- 3-(2-methyl-6- oxo-1,6- dihydropyridin-3- yl)-7- (trifluoromethyl)-2,3- dihydroquinazolin- 4(1H)-one and 1-((1R,3R)-3- fluorocyclopentyl)- 3-(2-methyl-6- oxo-1,6- dihydropyridin-3- yl)-7- (trifluoromethyl)- 2.3- dihydroquinazolin- 4(1H)-one Tested in the assay section as a 50/50 mixture of the two compounds | 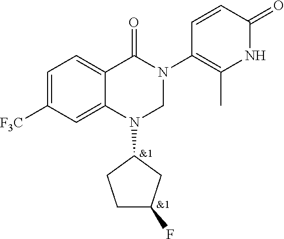 [SMILES: Cc1[nH]c(=O)ccc1N1CN([C@H]2CC[C@H](F)C2)c2cc(C(F)(F)F)ccc2C1=O] | ¹H NMR (METHANOL- d₄) δ: 7.88-8.21 (m, 1H), 7.53 (br d, J = 9.3 Hz, 1H), 7.33 (br s, 1H), 7.13-7.28 (m, 1H), 6.46 (dd, J = 9.5, 3.2 Hz, 1H), 5.28 (br s, 1H), 5.15 (br s, 1H), 4.73-4.85 (m, 2H), 4.49-4.70 (m, 1H), 2.21-2.35 (m, 5H), 1.94- 2.13 (m, 3H), 1.72-1.83 (m, 1H). MS (m/z) 410.4 (M + H)⁺ | 1-((1S,3S)-3- fluorocyclopentyl)- 3-(6-methoxy-2- methylpyridin-3- yl)-7- (trifluoromethyl)-2,3- dihydroquinazolin- 4(1H)-one 1-((1R,3R)-3- fluorocyclopentyl)- 3-(6-methoxy-2- methylpyridin-3- yl)-7- (trifluoromethyl)-2,3- dihydroquinazolin- 4(1H)-one |
| 209 | 3-(1,2-dimethyl- 6-oxo-1,6- dihydropyridin-3- yl)-1-(4-fluoro-2- methylphenyl)-6- (trifluoromethyl)- 2,3- dihydroquinazolin- 4(1H)-one | 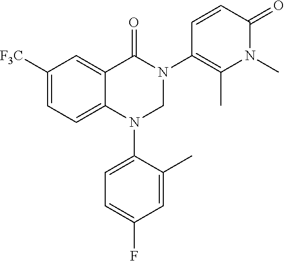 [SMILES: Cc1cc(F)ccc1N1CN(c2ccc(=O)n(C)c2C)C(=O)c2cc(C(F)(F)F)ccc21] | ¹H NMR (400 MHz, DMSO-d₆) δ: 8.09 (d, J = 1.96 Hz, 1H), 7.66 (dd, J = 8.56, 1.71 Hz, 1H), 7.39-7.47 (m, 1H), 7.29-7.38 (m, 1H), 7.12-7.26 (m, 1H). 6.23-6.45 (m, 2H), 5.55 (br d, J = 10.27 Hz, 1H), 5.06-5.24 (m, 1H), 4.72 (d, J = 9.29 Hz, 1H), 3.46 (s, 3H), 3.34 (s, 3H), 2.25 (s, 3H). MS (m/z) 446.3 (M + H)⁺ | 1-(4-fluoro-2- methylphenyl)- 3-(6-methoxy-2- methylpyridin-3-yl)-6- (trifluoromethyl)-2,3- dihydroquinazolin- 4(1H)-one |
| 210 | 3-(2-chloro-6- oxo-1,6- dihydropyridin-3- yl)-1-(4-fluoro-2- methylphenyl)-6- (trifluoromethyl)-2,3- dihydroquinazolin- 4(1H)-one | 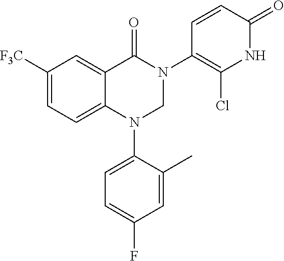 [SMILES: Cc1cc(F)ccc1N1CN(c2ccc(=O)[nH]c2Cl)C(=O)c2cc(C(F)(F)F)ccc21] | ¹H NMR (400 MHz, DMSO-d₆) δ: 11.76 (br s, 1H), 8.10 (br s, 1H), 7.84 (br d, J = 7.34 Hz, 1H), 7.58- 7.75 (m, 1H), 7.37-7.55 (m, 1H), 7.27-7.37 (m, 1H), 7.00-7.27 (m, 1H), 6.73 (br d, J = 8.31 Hz, 1H), 6.27-6.51 (m, 1H), 5.44-5.68 (m, 1H), 4.75-5.03 (m, 1H), 2.25 (s, 3H). MS (m/z) 452.3 (M + H)⁺ | 3-(2-chloro-6- methoxypyridin- 3-yl)-1-(4- fluoro-2- methylphenyl)-6- (trifluoromethyl)-2,3- dihydroquinazolin- 4(1H)-one |
| 211 | 3-(2-bromo-4- methyl-6-oxo-1,6- dihydropyridin-3- yl)-1-(4-fluoro-2- methylphenyl)-7- (trifluoromethyl)-2,3- dihydroquinazolin- 4(1H)-one | 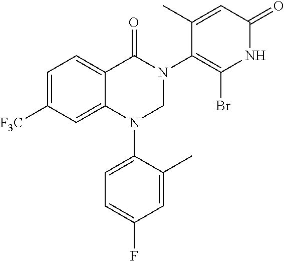 [SMILES: Cc1cc(F)ccc1N1CN(c2c(C)cc(=O)[nH]c2Br)C(=O)c2ccc(C(F)(F)F)cc21] | ¹H NMR (400 MHz, DMSO-d₆) δ: 11.67 (br s, 1H), 8.08 (d, J = 7.8 Hz, 1H), 7.51-7.38 (m, 1H), 7.34-7.15 (m, 3H) 6.62 (s, 1H), 6.36 (s, 1H), 5.57-5.43 (m, 1H) 5.04-4.83 (m, 1H), 2.30-2.18 (m, 6H) MS (m/z) 509.8 (M)⁺ | 3-(2-bromo-6- methoxy-4- methylpyridin-3- yl)-1-(4-fluoro-2- methylphenyl)-7- (trifluoromethyl)-2,3- dihydroquinazolin- 4(1H)-one |
| 212 | 1-(4-fluoro-2- isopropylphenyl)- 3-(2-methyl-6- oxo-1,6- dihydropyridin-3- yl)-7- (trifluoromethyl)-2,3- dihydropyrido[2,3- d]pyrimidin- 4(1H)-one | 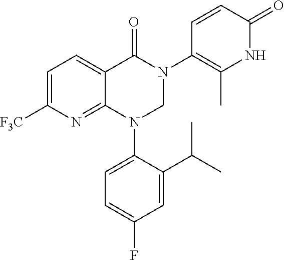 [SMILES: Cc1[nH]c(=O)ccc1N1CN(c2ccc(F)cc2C(C)C)c2nc(C(F)(F)F)ccc2C1=O] | ¹H NMR (400 MHz, DMSO-d₆) δ: 11.83 (br s, 1H), 8.37 (d, J = 7.3 Hz, 1H), 7.48-7.26 (m, 4H), 7.19-7.09 (m, 1H), 6.30-6.14 (m, 1H), 5.64 (d, J = 9.8 Hz, 0.6H), 5.29 (d, J = 9.8 Hz, 0.4H), 5.13 (d, J = 9.8 Hz, 0.4H), 4.84 (d, J = 9.8 Hz, 0.6H), 3.12- 2.91 (m, 1H), 2.22- 2.10 (m, 3H), 1.22- 1.03 (m, 6H). MS (m/z) 461.3 (M + H)⁺ | 1-(4-fluoro-2- isopropylphenyl)- 3-(6-methoxy- 2-methylpyridin- 3-yl)-7- (trifluoromethyl)-2,3- dihydropyrido[2,3- d]pyrimidin- 4(1H)-one |
| 213 | 6-chloro-1-(4- fluoro-2- isopropylphenyl)- 3-(2-methyl-6- oxo-1,6- dihydropyridin-3- yl)-2,3- dihydropyrido[2,3- d]pyrimidin- 4(1H)-one | 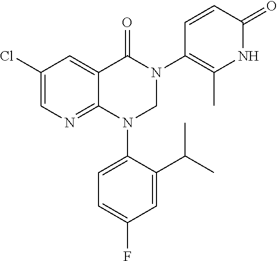 [SMILES: Cc1[nH]c(=O)ccc1N1CN(c2ccc(F)cc2C(C)C)c2ncc(Cl)cc2C1=O] | ¹H NMR (400 MHz, DMSO-d₆) δ: 11.81 (br s, 1H), 8.26 (d, J = 2.9 Hz, 1H), 8.11 (d, J = 2.9 Hz, 1H), 7.43-7.26 (m, 3H), 7.20-7.03 (m, 1H), 6.22 (br d, J = 9.3 Hz, 1H), 5.57 (d, J = 9.8 Hz, 0.6H), 5.28 (d, J = 10.3 Hz, 0.4H), 5.02 (d, J = 10.3 Hz, 0.4H), 4.76 (d, J = 9.8 Hz, 0.6H), 3.15- 2.97 (m, 1H), 2.19- 2.08 (m, 3H), 1.20- 1.07 (m, 6H). MS (m/z) 427.3 (M + H)⁺ | 6-chloro-1-(4- fluoro-2- isopropylphenyl)- 3-(6-methoxy- 2-methylpyridin- 3-yl)-2,3- dihydropyrido[2,3- d]pyrimidin- 4(1H)-one |
| 214 | 5-(1-(4-fluoro-2- methylphenyl)-4- thioxo-7- (trifluoromethyl)- 1,4- dihydroquinazolin- 3(2H)-yl)-6- methylpyridin- 2(1H)-one | 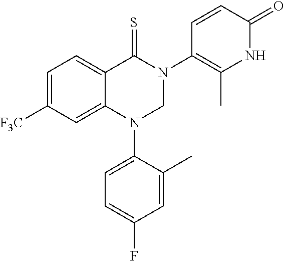 [SMILES: Cc1cc(F)ccc1N1CN(c2ccc(=O)[nH]c2C)C(=S)c2ccc(C(F)(F)F)cc21] | ¹H NMR (400 MHz, DMSO-d₆) δ: 11.87- 11.78 (m, 1H), 8.56- 8.50 (m, 1H), 7.40- 7.25 (m, 4H), 7.25- 7.15 (m, 1H), 6.57- 6.40 (m, 1H), 6.26- 6.18 (m, 1H), 5.48- 5.28 (m, 1H), 5.24- 5.05 (m, 1H), 2.26- 2.16 (m, 3H), 2.12- 1.96 (m, 3H). MS (m/z) 448.0 (M + H)⁺ | 1-(4-fluoro-2- methylphenyl)- 3-(6-methoxy-2- methylpyridin-3- yl)-7- (trifluoromethyl)-2,3- dihydroquinazoline- 4(1H)-thione |

### Example 215

### 1-(4-Fluoro-2-methylphenyl)-3-(6-oxo-1,6-dihydropyridin-3-yl)-6-(trifluoromethyl)-2,3-dihydroquinazolin-4(1H)-one

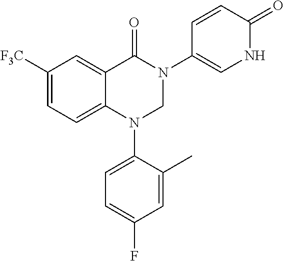 [SMILES: Cc1cc(F)ccc1N1CN(c2ccc(=O)[nH]c2)C(=O)c2cc(C(F)(F)F)ccc21]

To a stirring solution of 1-(4-fluoro-2-methylphenyl)-3-(6-methoxypyridin-3-yl)-6-(trifluoromethyl)-2,3-dihydroquinazolin-4(1H)-one (110 mg, 0.255 mmol) in DMF (10 mL) was added 4-methylbenzenesulfonic acid hydrate (340 mg, 1.785 mmol) followed by lithium chloride (76 mg, 1.785 mmol) at 0° C. The reaction mixture was stirred at 120° C. for 16 hours and then cooled to 25° C. The reaction was quenched with ice water (20 mL) and extracted with EtOAc (2×50 mL). The combined organic extracts were washed with brine (10 mL), dried over Na₂SO₄, filtered and concentrated under reduced pressure. The resultant dark brown liquid was purified by column chromatography (Biotage, 10 g SNAP column, 0-5% MeOH/DCM) to give the title compound as an off-white solid (84 mg, 0.197 mmol, 77% yield). ¹H NMR (400 MHz, DMSO-d₆) δ: 11.75 (s, 1H), 8.08 (d, J=2.00 Hz, 1H), 7.64 (dd, J=2.40, 8.80 Hz, 1H), 7.54 (s, 1H), 7.50 (dd, J=2.80, 9.60 Hz, 1H), 7.40-7.41 (m, 1H), 7.33 (dd, J=3.20, 9.80 Hz, 1H), 7.18-7.19 (m, 1H), 6.32-6.34 (m, 2H), 5.41 (d, J=9.60 Hz, 1H), 5.05 (d, J=8.80 Hz, 1H), 2.23 (s, 1H). MS (m/z) 417.9 (M+H)⁺.

Examples 216-239 were prepared from the indicated intermediate by methods analogous to those described for Example 215.

| Ex. | Name | Structure | Characterization | Intermediate |
|---|---|---|---|---|
| 216 | 1-(4-fluoro-2- methoxyphenyl)- 3-(6-oxo-1,6- dihydropyridin-3- yl)-7- (trifluoromethyl)- 2,3- dihydroquinazolin- 4(1H)-one | 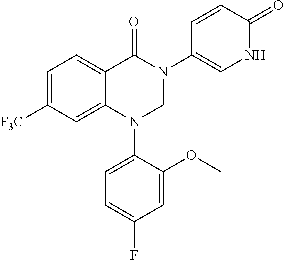 [SMILES: COc1cc(F)ccc1N1CN(c2ccc(=O)[nH]c2)C(=O)c2ccc(C(F)(F)F)cc21] | ¹H NMR (400 MHz, DMSO-d₆) δ: 11.74 (s, 1H), 8.04 (d, J = 8.00 Hz, 1H), 7.52 (d, J = 2.80 Hz, 1H), 7.49 (dd, J = 3.20, 9.60 Hz, 1H), 7.41 (dd, J = 6.00, 8.60 Hz, 1H), 7.22 (dd, J = 0.80, 8.20 Hz, 1H), 7.17 (dd, J = 2.80, 11.20 Hz, 1H), 6.89-6.89 (m, 1H), 6.50 (s, 1H), 6.35 (d, J = 9.60 Hz, 1H), 5.18 (s, 2H), 3.79 (s, 3H). MS (m/z) 434.0 (M + H)⁺ | 1-(4-fluoro-2- methoxyphenyl)- 3-(6- methoxypyridin- 3-yl)-7- (trifluoromethyl)- 2,3- dihydroquinazolin- 4(1H)-one |
| 217 | 1-(4-fluoro-2- methylphenyl)-3- (6-oxo-1,6- dihydropyridin-3- yl)-5- (trifluoromethyl)- 2,3- dihydroquinazolin- 4(1H)-one | 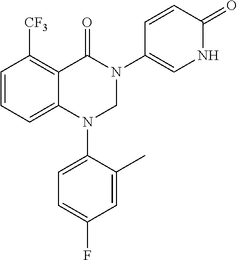 [SMILES: Cc1cc(F)ccc1N1CN(c2ccc(=O)[nH]c2)C(=O)c2c1cccc2C(F)(F)F] | ¹H NMR (400 MHz, DMSO-d₆) δ: 11.70 (s, 1H), 7.45-7.46 (m, 2H), 7.41 (dd, J = 2.80, 9.60 Hz, 1H), 7.36 (d, J = 7.60 Hz, 1H), 7.28-7.29 (m, 2H), 7.12-7.12 (m, 1H), 6.64 (d, J = 8.40 Hz, 1H), 6.33 (d, J = 9.60 Hz, 1H), 2.22 (s, 3H). MS (m/z) 417.9 (M + H)⁺ | 1-(4-fluoro-2- methylphenyl)-3- (6- methoxypyridin- 3-yl)-5- (trifluoromethyl)- 2,3- dihydroquinazolin- 4(1H)-one |
| 218 | 1-(4-fluoro-2- methylphenyl)-3- (5-methyl-6-oxo- 1,6- dihydropyridin-3- yl)-7- (trifluoromethyl)- 2,3- dihydroquinazolin- 4(1H)-one | 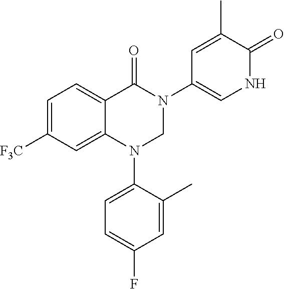 [SMILES: Cc1cc(F)ccc1N1CN(c2c[nH]c(=O)c(C)c2)C(=O)c2ccc(C(F)(F)F)cc21] | ¹H NMR (400 MHz, DMSO-d₆) δ: 11.66 (br. s., 1H), 8.07 (d, J = 8.0 Hz, 1H), 7.45-7.36 (m, 3H), 7.33 (dd, J = 9.7, 2.8 Hz, 1H), 7.26-7.15 (m, 2H), 6.37 (s, 1H), 5.35 (br. s., 1H), 5.03 (br. s., 1H), 2.23 (s, 3H), 1.97 (s, 3H). MS (m/z) 431.9 (M + H)⁺ | 1-(4-fluoro-2- methylphenyl)-3- (6-methoxy-5- methylpyridin-3- yl)-7- (trifluoromethyl)- 2,3- dihydroquinazolin- 4(1H)-one |
| 219 | 1-(4-fluoro-2- methylphenyl)-3- (6-oxo-1,6- dihydropyridazin- 3-yl)-7- (trifluoromethyl)- 2,3- dihydroquinazolin- 4(1H)-one | 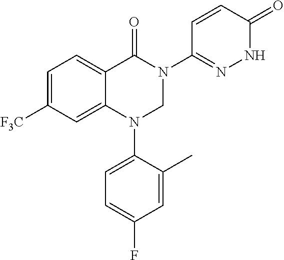 [SMILES: Cc1cc(F)ccc1N1CN(c2ccc(=O)[nH]n2)C(=O)c2ccc(C(F)(F)F)cc21] | ¹H NMR (400 MHz, DMSO-d₆) δ: 12.89 (s, 1H), 8.14 (d, J = 8.1 Hz, 1H), 7.82 (d, J = 10.0 Hz, 1H), 7.47 (dd, J = 8.7, 5.4 Hz, 1H), 7.33 (dd, J = 9.8, 3.0 Hz, 1H), 7.27 (dd, J = 8.2, 1.2 Hz, 1H), 7.21 (td, J = 8.4, 3.1 Hz, 1H), 6.94 (d, J = 10.1 Hz, 1H), 6.41 (s, 1H), 5.50 (br. s., 1H), 5.28 (br. s., 1H), 2.19 (s, 3H). MS (m/z) 419.0 (M + H)⁺ | 1-(4-fluoro-2- methylphenyl)-3- (6- methoxypyridazin- 3-yl)-7- (trifluoromethyl)- 2,3- dihydroquinazolin- 4(1H)-one |
| 220 | 1-(4- fluorobenzyl)-3- (6-oxo-1,6- dihydropyridin-3- yl)-7- (trifluoromethyl)- 2,3- dihydroquinazolin- 4(1H)-one | 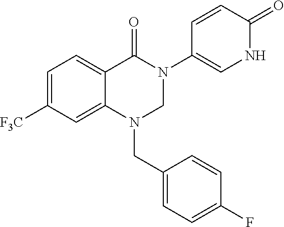 [SMILES: O=C1c2ccc(C(F)(F)F)cc2N(Cc2ccc(F)cc2)CN1c1ccc(=O)[nH]c1] | ¹H NMR (400 MHz, DMSO-d₆) δ: 11.73 (s, 1H), 7.98 (d, J = 8.00 Hz, 1H), 7.50 (d, J = 2.80 Hz, 1H), 7.41-7.42 (m, 3H), 7.14-7.16 (m, 4H), 6.37 (dd, J = 0.40, 9.60 Hz, 1H), 5.02 (s, 2H), 4.73 (s, 2H). MS (m/z) 418.0 (M + H)⁺ | 1-(4- fluorobenzyl)-3- (6- methoxypyridin- 3-yl)-7- (trifluoromethyl)- 2,3- dihydroquinazolin- 4(1H)-one |
| 221 | 1-(4-fluoro-2- methylphenyl)-3- (4-methyl-6-oxo- 1,6- dihydropyridin-3- yl)-7- (trifluoromethyl)- 2,3- dihydroquinazolin- 4(1H)-one | 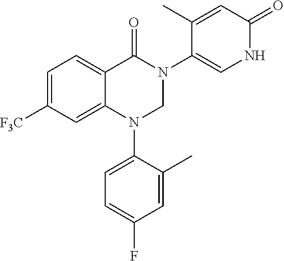 [SMILES: Cc1cc(=O)[nH]cc1N1CN(c2ccc(F)cc2C)c2cc(C(F)(F)F)ccc2C1=O] | ¹H NMR (400 MHz, DMSO-d₆) δ: 11.58 (br. s., 1H), 8.07 (d, J = 8.0 Hz, 1H), 7.49 (s, 1H), 7.42-7.30 (m, 2H), 7.27- 7.15 (m, 2H), 6.51- 6.29 (m, 1H), 6.25 (s. 1H), 5.52 (br. s., 0.5H), 5.16 (br. s., 1H), 4.80 (br. s., 0.5H), 2.22 (br. s., 3H), 2.03 (br. s., 3H). MS (m/z) 432.0 (M + H)⁺ | 1-(4-fluoro-2- methylphenyl)-3- (6-methoxy-4- methylpyridin-3- yl)-7- (trifluoromethyl)- 2,3- dihydroquinazolin- 4(1H)-one |
| 222 | 1-(4-fluoro-2- methylphenyl)-4- oxo-3-(6-oxo- 1,6- dihydropyridin-3- yl)-1,2,3,4- tetrahydro- quinazoline-7- carbonitrile | 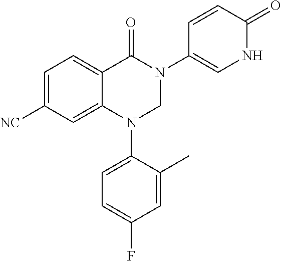 [SMILES: Cc1cc(F)ccc1N1CN(c2ccc(=O)[nH]c2)C(=O)c2ccc(C#N)cc21] | ¹H NMR (400 MHz, DMSO-d₆) δ: 11.73 (s, 1H), 8.00 (d, J = 8.0 Hz, 1H), 7.53 (d, J = 2.8 Hz, 1H), 7.49 (dd, J = 9.6, 2.8 Hz, 1H), 7.37 (dd, J = 5.60, 8.80 Hz, 1H), 7.31 (dd, J = 8.0, 1.6 Hz, 2H), 7.19-7.18 (m, 1H), 6.57 (d, J = 1.2 Hz, 1H), 6.36 (d, J = 9.6 Hz, 1H), 5.34 (s, 1H), 5.05 (s,1H), 2.23 (s, 3H). MS (m/z) 375.0 (M + H)⁺ | 1-(4-fluoro-2- methylphenyl)-3- (6- methoxypyridin- 3-yl)-4-oxo- 1,2,3,4-tetra- hydroquinazoline- 7-carbonitrile |
| 223 | 1-(4-fluoro-2- methoxyphenyl)- 3-(6-oxo-1,6- dihydropyridin-3- yl)-5- (trifluoromethyl)- 2,3- dihydroquinazolin- 4(1H)-one | 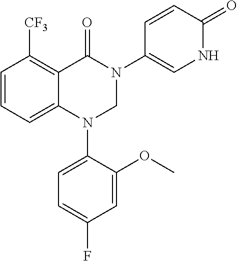 [SMILES: COc1cc(F)ccc1N1CN(c2ccc(=O)[nH]c2)C(=O)c2c1cccc2C(F)(F)F] | ¹H NMR (400 MHz, DMSO-d₆) δ: 11.72 (s, 1H), 7.37-7.36 (m, 2H), 7.34-7.32 (m, 3H), 7.13 (dd, J = 2.80, 10.80 Hz, 1H), 6.84-6.83 (m, 1H), 6.74-6.76 (m, 1H), 6.33 (d, J = 9.60 Hz, 1H), 5.07 (s, 2H), 3.78 (s, 3H). MS (m/z) 434.0 (M + H)⁺ | 1-(4-fluoro-2- methoxyphenyl)- 3-(6- methoxypyridin- 3-yl)-5- (trifluoromethyl)- 2,3- dihydroquinazolin- 4(1H)-one |
| 224 | 3-(2-methyl-6- oxo-1,6- dihydropyridin-3- yl)-1-(4- methylthiazol-5- yl)-6- (trifluoromethyl)- 2,3- dihydroquinazolin- 4(1H)-one | 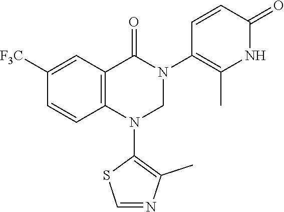 [SMILES: Cc1ncsc1N1CN(c2ccc(=O)[nH]c2C)C(=O)c2cc(C(F)(F)F)ccc21] | ¹H NMR (400 MHz, DMSO-d₆) δ:11.80 (s, 1H), 9.10 (s, 1H), 8.10 (d, J = 2.00 Hz, 1H), 7.76 (dd, J = 8.80, 2.00 Hz, 1H), 7.40 (d, J = 9.60 Hz, 1H), 6.63 (d, J = 8.40 Hz, 1H), 6.21 (d, J = 9.60 Hz, 1H), 5.29 (d, J = 9.60 Hz, 1H), 5.00 (d, J = 9.60 Hz, 1H), 2.29 (s, 3H), 2.14 (s, 3H). MS (m/z) 420.8 (M)⁺ | 3-(6-methoxy-2- methylpyridin-3- yl)-1-(4- methylthiazol-5- yl)-6- (trifluoromethyl)- 2,3- dihydroquinazolin- 4(1H)-one |
| 225 | 1-(2,4- dimethoxyphenyl)- 3-(2-methyl-6- oxo-1,6- dihydropyridin-3- yl)-6- (trifluoromethyl)- 2,3- dihydroquinazolin- 4(1H)-one | 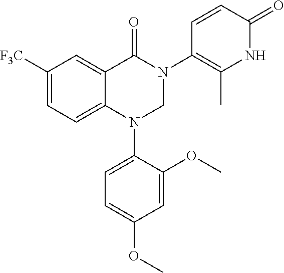 [SMILES: COc1ccc(N2CN(c3ccc(=O)[nH]c3C)C(=O)c3cc(C(F)(F)F)ccc32)c(OC)c1] | ¹H NMR (400 MHz, DMSO-d₆) δ 11.77 (br s, 1H), 8.04 (d, J = 2.4 Hz, 1H), 7.61 (dd, J = 8.8, 2.4 Hz, 1H), 7.40 (d, J = 9.3 Hz, 1H), 7.29 (d, J = 8.3 Hz, 1H), 6.77 (d, J = 2.9 Hz, 1H), 6.63 (dd, J = 8.6, 2.7 Hz, 1H), 6.43 (d, J = 8.8 Hz, 1H), 6.19 (d, J = 9.8 Hz, 1H), 5.26 (br d, J = 10.3 Hz, 1H), 4.81 (br d, J = 9.8 Hz, 1H), 3.82 (s, 3H), 3.77 (s, 3H), 2.12 (s, 3H). MS (m/z) 460.3 (M + H)⁺ | 1-(2,4- dimethoxyphenyl)- 3-(6-methoxy- 2-methylpyridin- 3-yl)-6- (trifluoromethyl)- 2,3- dihydroquinazolin- 4(1H)-one |
| 226 | 1-(4-fluoro-2- methoxyphenyl)- 3-(2-methyl-6- oxo-1,6- dihydropyridin-3- yl)-6- (trifluoromethyl)- 2,3- dihydroquinazolin- 4(1H)-one | 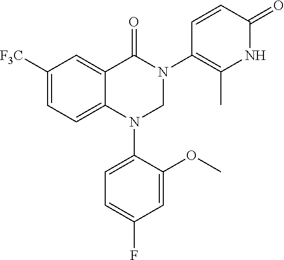 [SMILES: COc1cc(F)ccc1N1CN(c2ccc(=O)[nH]c2C)C(=O)c2cc(C(F)(F)F)ccc21] | H NMR (400 MHz, DMSO-d₆) δ 12.01- 11.71 (m, 1H), 8.08- 8.04 (m, 1H), 7.63 (dd, J = 8.8, 2.0 Hz, 1H), 7.48- 7.35 (m, 2H), 7.17 (dd, J = 11.2, 2.9 Hz, 1H), 6.90 (td, J = 8.4, 2.7 Hz, 1H), 6.47 (d, J = 8.8 Hz, 1H), 6.20 (d, J = 9.3 Hz, 1H), 5.29 (d, J = 9.8 Hz, 1H), 4.96-4.91 (m, 1H), 3.79 (s, 3H), 2.11 (s, 3H) MS (m/z) 448.2 (M + H)⁺ | 1-(4-fluoro-2- methoxyphenyl)- 3-(6-methoxy-2- methylpyridin-3- yl)-6- (trifluoromethyl)- 2,3- dihydroquinazolin- 4(1H)-one |
| 227 | 7-chloro-6- fluoro-1-(4- fluoro-2- methylphenyl)-3- (2-methyl-6-oxo- 1,6- dihydropyridin-3- yl)-2,3- dihydropyrido[2, 3-d]pyrimidin- 4(1H)-one | 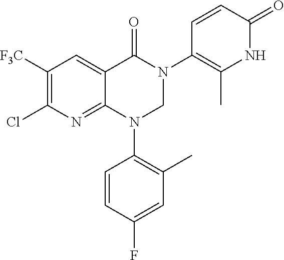 | ¹H NMR (400 MHz, DMSO-d₆) δ (ppm) = 8.33 (s, 1H), 8.17 (d, J = 7.9 Hz, 1H), 7.43-7.31 (m, 2H), 7.24 (dd, J = 3.0, 9.8 Hz, 1H), 7.18- 7.09 (m, 1H), 6.20 (d, J = 9.8 Hz, 1H), 5.59-4.79 (m, 2H), 2.21 (s, 3H), 2.11 (s, 3H). MS (m/z) 417 (M + H)⁺ | 7-chloro-6- fluoro-1-(4- fluoro-2- methylphenyl)-3- (6-methoxy-2- methylpyridin-3- yl)-2,3- dihydropyrido[2, 3-d]pyrimidin- 4(1H)-one |
| 228 | 3-(2-methyl-6- oxo-1,6- dihydropyridin-3- yl)-1-(3- methylthiophen- 2-yl)-6- (trifluoromethyl)- 2,3- dihydroquinazolin- 4(1H)-one | 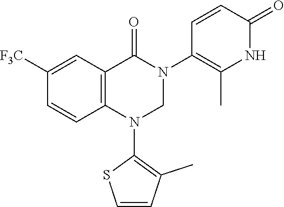 [SMILES: Cc1ccsc1N1CN(c2ccc(=O)[nH]c2C)C(=O)c2cc(C(F)(F)F)ccc21] | ¹H NMR (400 MHz, DMSO-d₆) δ: 11.81 (s, 1H), 8.09 (d, J = 2.4 Hz, 1H), 7.75 (dd, J = 1.60, 8.80 Hz, 1H), 7.50 (d, J = 6.00 Hz, 1H), 7.40 (d, J = 9.60 Hz, 1H), 7.02 (d, J = 5.60 Hz, 1H), 6.59 (d, J = 8.80 Hz, 1H), 6.21 (d, J = 9.60 Hz, 1H), 5.31 (d, J = 9.60 Hz, 1H), 4.96 (d, J = 9.60 Hz, 1H), 2.14 (s, 3H), 2.09 (s, 3H). MS (m/z) 419.8 (M + H)⁺ | 3-(6-methoxy-2- methylpyridin-3- yl)-1-(3- methylthiophen- 2-yl)-6- (trifluoromethyl)- 2,3- dihydroquinazolin- 4(1H)-one |
| 229 | 1-(4-fluoro-2- methoxyphenyl)- 4-oxo-3-(6-oxo- 1,6- dihydropyridin-3- yl)-1,2,3,4-tetra- hydroquinazoline- 7-carbonitrile | 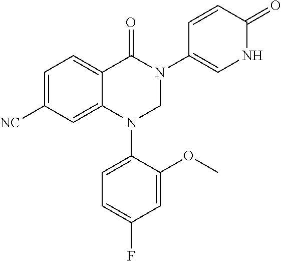 [SMILES: COc1cc(F)ccc1N1CN(c2ccc(=O)[nH]c2)C(=O)c2ccc(C#N)cc21] | ¹H NMR (400 MHz, DMSO-d₆) δ: 11.76 (br s, 1H), 7.97 (d, J = 8.0 Hz, 1H), 7.52 (d, J = 2.8 Hz, 1H), 7.48 (dd, J = 2.8, 9.6 Hz, 1H), 7.39 (dd, J = 6.4, 8.8 Hz, 1H), 7.29 (dd, J = 1.6, 8.0 Hz, 1H), 7.15 (dd, J = 2.8, 11.2 Hz, 1H), 6.89 (td, J = 2.8, 8.4 Hz, 1H), 6.69 (d, J = 1.2 Hz, 1H), 6.35 (d, J = 9.6 Hz, 1H), 5.16 (s, 2H), 3.80 (s, 3H). MS (m/z) 391.0 (M + H)⁺ | 1-(4-fluoro-2- methoxyphenyl)- 3-(6- methoxypyridin- 3-yl)-4-oxo- 1,2,3,4-tetra- hydroquinazoline- 7-carbonitrile |
| 230 | 1-(4-fluoro-2- methylphenyl)-3- (2-methyl-6-oxo- 1,6- dihydropyridin-3- yl)-7-(2,2,2- trifluoroethoxy)- 2,3- dihydroquinazolin- 4(1H)-one | 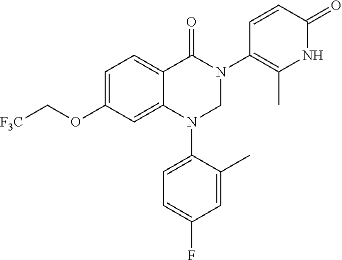 [SMILES: Cc1cc(F)ccc1N1CN(c2ccc(=O)[nH]c2C)C(=O)c2ccc(OCC(F)(F)F)cc21] | ¹H NMR (400 MHz, DMSO-d₆) δ: 11.75 (br s, 1H), 7.84 (d, J = 8.6 Hz, 1H), 7.38-7.26 (m, 3H), 7.22-7.13 (m, 1H), 6.65 (dd, J = 8.7, 2.2 Hz, 1H), 6.17 (d, J = 9.6 Hz, 1H), 5.83-5.64 (m, 1H), 5.38 (br s, 0.6H), 5.11-4.91 (m, 0.8H), 4.76-4.60 (m, 2.6H), 2.24 (s, 3H), 2.10 (br s, 3H). MS (m/z) 462.0 (M + H)⁺ | 1-(4-fluoro-2- methylphenyl)-3- (6-methoxy-2- methylpyridin-3- yl)-7-(2,2,2- trifluoroethoxy)- 2,3- dihydroquinazolin- 4(1H)-one |
| 231 | 1-(2-methyl-4- (trifluoromethoxy) phenyl)-3-(6- oxo-1,6- dihydropyridin-3- yl)-6- (trifluoromethyl)- 2,3- dihydroquinazolin- 4(1H)-one | 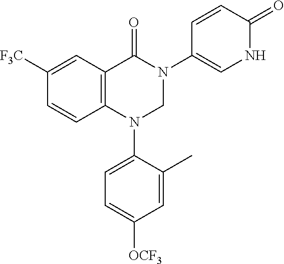 [SMILES: Cc1cc(OC(F)(F)F)ccc1N1CN(c2ccc(=O)[nH]c2)C(=O)c2cc(C(F)(F)F)ccc21] | ¹H-NMR (400 MHz, DMSO-d₆): δ 11.75 (br s, 1H), 8.09 (d, J = 2.40 Hz, 1H), 7.66 (dd, J = 2.00 Hz, 8.80 Hz, 1H), 7.54- 7.48 (m, 4H), 7.38-7.35 (m, 1H), 6.38-6.35 (m, 2H), 5.43 (br s, 1H), 5.10 (br s, 1H), 2.25 (s, 3H). MS (m/z) 483.8 (M)⁺ | 3-(6- methoxypyridin- 3-yl)-1-(2- methyl-4- (trifluoromethoxy) phenyl)-6- (trifluoromethyl)- 2,3- dihydroquinazolin- 4(1H)-one |
| 232 | 6-chloro-3-(4- chloro-2-methyl- 6-oxo-1,6- dihydropyridin-3- yl)-1-(4-fluoro-2- methylphenyl)- 2,3- dihydroquinazolin- 4(1H)-one | 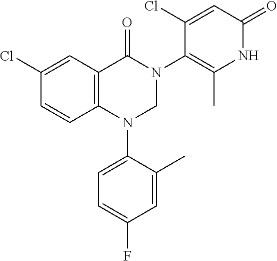 [SMILES: Cc1cc(F)ccc1N1CN(c2c(Cl)cc(=O)[nH]c2C)C(=O)c2cc(Cl)ccc21] | ¹H-NMR (400 MHz, DMSO-d₆): δ 12.08 (s, 1H), 7.79 (d, J = 2.80 Hz, 1H), 7.42-7.27 (m, 3H), 7.16 (t, J = 8.40 Hz, 1H), 6.44 (s, 1H), 6.26-6.25 (m, 1H), 5.40-4.79 (m, 2H), 2.22 (s, 3H), 2.17 (d, J = 15.20 Hz, 3H). MS (m/z) 432.0 (M + H)⁺ | 6-chloro-3-(4- chloro-6- methoxy-2- methylpyridin-3- yl)-1-(4-fluoro-2- methylphenyl)- 2,3- dihydroquinazolin- 4(1H)-one |
| 233 | 7-chloro-1-(4- fluoro-2- methylphenyl)-3- (2-methyl-6-oxo- 1,6- dihydropyridin-3- yl)-4-oxo- 1,2,3,4-tetra- hydroquinazoline- 6-carbonitrile | 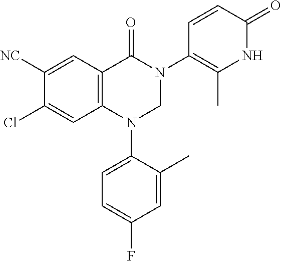 [SMILES: Cc1cc(F)ccc1N1CN(c2ccc(=O)[nH]c2C)C(=O)c2cc(C#N)c(Cl)cc21] | ¹H-NMR (400 MHz, DMSO-d₆): δ 11.79 (s, 1H), 8.47-8.24 (m, 1H), 7.47-7.34 (m, 3H), 7.25- 7.22 (m, 1H), 6.32-6.22 (m, 1H), 6.21-6.18 (m, 1H), 5.54-4.83 (m, 2H), 2.24 (s, 3H), 2.11 (s, 3H). MS (m/z) 422.8 (M)⁺ | 7-chloro-1-(4- fluoro-2- methylphenyl)-3- (6-methoxy-2- methylpyridin-3- yl)-4-oxo- 1,2,3,4-tetra- hydroquinazoline- 6-carbonitrile |
| 234 | 6-chloro-1-(4- fluoro-2- methylphenyl)-5- methyl-3-(2- methyl-6-oxo- 1,6- dihydropyridin-3- yl)-2,3- dihydroquinazolin- 4(1H)-one | 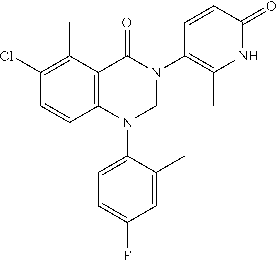 [SMILES: Cc1cc(F)ccc1N1CN(c2ccc(=O)[nH]c2C)C(=O)c2c1ccc(Cl)c2C] | ¹H NMR (400 MHz, DMSO-d₆) δ: 11.75 (br s, 1H), 7.37 (d, J = 8.9 Hz, 1H), 7.32-7.19 (m, 3H), 7.16-7.10 (m, 1H), 6.17 (d, J = 9.4 Hz, 2H), 5.28- 4.70 (m, 2H), 2.64 (s, 3H), 2.20 (s, 3H), 2.07 (br s, 3H). MS (m/z) 412.0 (M + H)⁺ | 6-chloro-1-(4- fluoro-2- methylphenyl)-3- (6-methoxy-2- methylpyridin-3- yl)-5-methyl-2,3- dihydroquinazolin- 4(1H)-one |
| 235 | 1-(3,5-difluoro-2- methylphenyl)-3- (2-methyl-6-oxo- 1,6- dihydropyridin-3- yl)-6- (trifluoromethyl)- 2,3- dihydroquinazolin- 4(1H)-one | 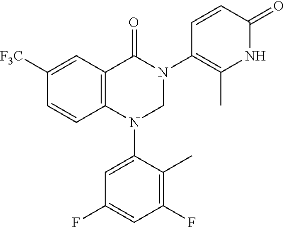 [SMILES: Cc1[nH]c(=O)ccc1N1CN(c2cc(F)cc(F)c2C)c2ccc(C(F)(F)F)cc2C1=O] | ¹H NMR (400 MHz, DMSO-d₆) δ: 11.80 (s, 1H), 8.09 (d, J = 1.60 Hz, 1H), 7.71 (dd, J = 2.00, 8.60 Hz, 1H), 7.52-7.38 (m, 3H), 6.66 (d, J = 8.40 Hz, 1H), 6.19 (d, J = 8.80 Hz, 1H), 5.36 (d, J = 10.00 Hz, 1H), 5.03 (d, J = 10.00 Hz, 1H), 2.23 (s, 3H), 2.08 (s, 3H). MS (m/z) 450.0 (M + H)⁺ | 1-(3,5-difluoro-2- methylphenyl)-3- (6-methoxy-2- methylpyridin-3- yl)-6- (trifluoromethyl)- 2,3- dihydroquinazolin- 4(1H)-one |
| 236 | 1-(4-fluoro-2- isopropylphenyl)- 5-methoxy-3-(2- methyl-6-oxo- 1,6- dihydropyridin-3- yl)-2,3- dihydroquinazolin- 4(1H)-one | 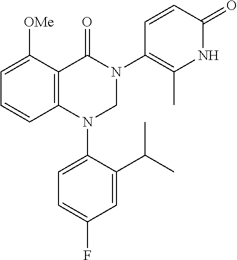 [SMILES: COc1cccc2c1C(=O)N(c1ccc(=O)[nH]c1C)CN2c1ccc(F)cc1C(C)C] | ¹H NMR (400 MHz, DMSO-d₆) δ: 11.72 (br s, 1H), 7.36-7.18 (m, 4H), 7.13 (td, J = 8.3, 2.8 Hz, 1H), 6.56 (d, J = 8.4 Hz, 1H), 6.16 (d, J = 9.5 Hz, 1H), 5.77 (dd, J = 17.9, 8.2 Hz, 1H), 5.22 (d, J = 10.0 Hz, 0.6H), 5.06 (d, J = 10.6 Hz, 0.4H), 4.75 (d, J = 10.6 Hz, 0.4H), 4.58 (d, J = 9.9 Hz, 0.6H), 3.79 (s, 3H), 3.24-3.05 (m, 1H), 2.09 (d, J = 9.1 Hz, 3H), 1.19-1.09 (m, 6H). MS (m/z) 422.2 (M + H)⁺ | 1-(4-fluoro-2- isopropylphenyl)- 5-methoxy-3-(6- methoxy-2- methylpyridin-3- yl)-2,3- dihydroquinazolin- 4(1H)-one |
| 237 | 1-(bicyclo[1.1.1] pentan-1-yl)-3-(2- methyl-6-oxo- 1,6- dihydropyridin-3- yl)-7- (trifluoromethyl)- 2,3- dihydroquinazolin- 4(1H)-one | 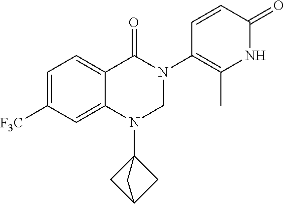 [SMILES: Cc1[nH]c(=O)ccc1N1CN(C23CC(C2)C3)c2cc(C(F)(F)F)ccc2C1=O] | ¹H NMR (400 MHz, DMSO-d₆) δ: 11.83 (br s, 1H), 8.03 (d, J = 8.0 Hz, 1H), 7.39-7.31 (m, 3H), 6.22 (d, J = 9.5 Hz, 1H), 4.94-4.87 (m, 1H), 4.79- 4.71 (m, 1H), 2.53 (br s, 1H), 2.12 (s, 6H), 2.09 (s, 3H) MS (m/z) 390.2 (M + H)⁺ | 1-(bicyclo[1.1.1] pentan-1-yl)-3-(6- methoxy-2- methylpyridin-3- yl)-7- (trifluoromethyl)- 2,3- dihydroquinazolin- 4(1H)-one |
| 238 | 1-(4-fluoro-2- methylphenyl)-3- (3-methyl-2-oxo- 1,2- dihydropyridin-4- yl)-6- (trifluoromethyl)- 2,3- dihydroquinazolin- 4(1H)-one | 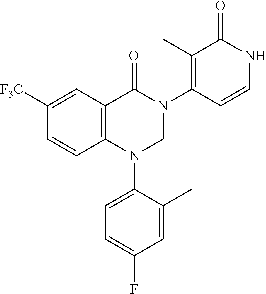 [SMILES: Cc1cc(F)ccc1N1CN(c2cc[nH]c(=O)c2C)C(=O)c2cc(C(F)(F)F)ccc21] | ¹H NMR (400 MHz, DMSO-d₆) δ: 11.66 (s, 1H), 8.11 (d, J = 2.00 Hz, 1H), 7.67 (dd, J = 2.40, 8.80 Hz, 1H), 7.40 (dd, J = 5.20, 8.60 Hz, 1H), 7.29-7.33 (m, 2H), 7.17- 7.22 (m, 1H), 6.38 (d, J = 8.80 Hz, 1H), 6.17 (d, J = 6.80 Hz, 1H), 5.54 (br s, 1H), 4.89 (br s, 1H), 2.22 (s, 3H), 1.86 (s, 3H) MS (m/z) 431.8 (M)⁺ | 1-(4-fluoro-2- methylphenyl)-3- (2-methoxy-3- methylpyridin-4- yl)-6- (trifluoromethyl)- 2,3- dihydro- quinazolin- 4(1H)-one |
| 239 | 5-(1-(4-fluoro-2- methylphenyl)-6- (trifluoromethyl)- 1,4- dihydroquinazolin- 3(2H)-yl)-6- methylpyridin- 2(1H)-one | 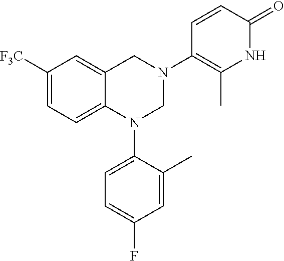 [SMILES: Cc1cc(F)ccc1N1CN(c2ccc(=O)[nH]c2C)Cc2cc(C(F)(F)F)ccc21] | ¹H NMR (400 MHz, DMSO-d₆) δ: 11.57 (s, 1H), 7.40 (s, 1H), 7.3- 7.09 (m, 5H), 6.12-6.08 (m, 2H), 4.60 (d, J = 11.20 Hz, 1H), 4.46 (d, J = 16.80 Hz, 1H), 4.27- 4.22 (m, 2H), 2.21 (s, 3H), 2.03 (s, 3H). MS (m/z) 418.0 (M + H)⁺ | 1-(4-fluoro-2- methylphenyl)-3- (6-methoxy-2- methylpyridin-3- yl)-6- (trifluoromethyl)- 1,2,3,4-tetra- hydroquinazoline |

### Example 240

### 3-(2-Methyl-6-oxo-1,6-dihydropyridin-3-yl)-1-(2-(2,2,2-trifluoroethyl)phenyl)-6-(trifluoromethyl)-2,3-dihydroquinazolin-4(1H)-one

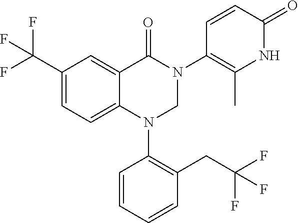 [SMILES: Cc1[nH]c(=O)ccc1N1CN(c2ccccc2CC(F)(F)F)c2ccc(C(F)(F)F)cc2C1=O]

A stirred suspension of 3-(6-methoxy-2-methylpyridin-3-yl)-1-(2-(2,2,2-trifluoroethyl)phenyl)-6-(trifluoromethyl)-2,3-dihydroquinazolin-4(1H)-one (11 mg, 0.022 mmol) and pyridin-1-ium bromide (35.5 mg, 0.222 mmol) in pyridine (1 mL) was heated in a sealed vial at 105° C. for 20 hours. The reaction mixture was then heated to 130° C. under microwave irradiation for 90 minutes. The reaction mixture was partially concentrated under a stream of nitrogen and the residue partitioned between water and EtOAc. The organic extract was dried by filtration through a hydrophobic frit and concentrated under a stream of nitrogen. The residue was purified using formic MDAP to give title product, 4 mg (8.41 μmol, 37% yield).

MS (m/z) 482 (M+H)⁺. ¹H NMR (400 MHz, CDCl₃) δ: 8.40 (d, J=2.0 Hz, 1H), 8.20-8.02 (br s, 1H), 7.67-7.46 (m, 4H), 7.39-7.30 (m, 2H), 6.50 (d, J=9.8 Hz, 1H), 6.32-6.22 (m, 1H), 5.51-5.30 (m, 1H), 4.81-4.58 (m, 1H), 3.70-3.54 (m, 1H), 3.46-3.32 (m, 1H), 2.35 (s, 3H)

Example 241 was prepared from the indicated intermediate by methods analogous to those described for Example 240.

| Ex. | Name | Structure | Characterization | Intermediate |
|---|---|---|---|---|
| 241 | 7- (difluoromethoxy)- 1-(4-fluoro-2- methylphenyl)-3- (2-methyl-6-oxo- 1,6-dihydropyridin-3- yl)-2,3- dihydropyrido[2,3- d]pyrimidin- 4(1H)-one | 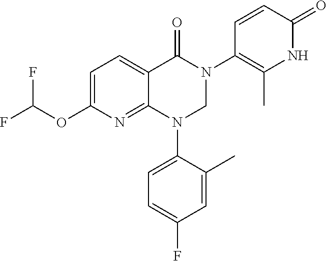 [SMILES: Cc1cc(F)ccc1N1CN(c2ccc(=O)[nH]c2C)C(=O)c2ccc(OC(F)F)nc21] | ¹H NMR (400 MHz, CDCl₃) δ: 12.92 (br s, 1H), 8.32 (d, J = 8.4 Hz, 1H), 7.34 (br d, J = 8.9 Hz, 1H), 7.09-7.18 (m, 1H), 7.05 (dd, J = 2.7, 9.1 Hz, 1H), 7.01-6.93 (m, 1H), 6.92 (t, J = 72.5 Hz, 1H), 6.46 (d, J = 9.4 Hz, 1H), 6.41 (d, J = 7.9 Hz, 1H), 5.47-4.64 (m, 2H), 2.34 (s, 3H), 2.25 (s, 3H). MS (m/z) 431.0 (M + H)⁺ | 7- (difluoromethoxy)- 1-(4-fluoro-2- methylphenyl)-3- (6-methoxy-2- methylpyridin-3- yl)-2,3- dihydropyrido[2,3- d]pyrimidin- 4(1H)-one |

Examples 242-243 were prepared from the indicated intermediate by methods analogous to those described for Intermediate 795

| Ex. | Name | Structure | Characterization | Intermediate |
|---|---|---|---|---|
| 242 | 6-fluoro-1-(4- fluoro-2- methylphenyl)-3- (2-methyl-6-oxo- 1,6- dihydropyridin-3- yl)-4-oxo- 1,2,3,4- tetrahydroquin- azoline-7- carbonitrile | 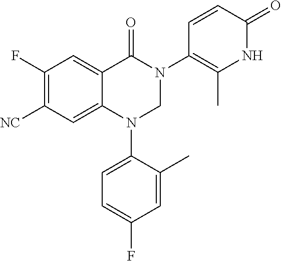 [SMILES: Cc1cc(F)ccc1N1CN(c2ccc(=O)[nH]c2C)C(=O)c2cc(F)c(C#N)cc21] | ¹H NMR (400 MHz, DMSO-d₆) δ: 11.72 (br. s., 1H), 7.83 (d, J = 9.3 Hz, 1H), 7.36-7.27 (m, 3H), 7.15 (t, 1.0 Hz, 1H), 6.72 (br. s., 1H), 6.19 (d, J = 9.3 Hz, 1H), 5.39- 4.81 (m, 2H), 2.23 (s, 3H), 2.07 (br. s., 3H). MS (m/z) 407.3 (M + H)⁺ | 6,7-difluoro-1-(4- fluoro-2- methylphenyl)-3- (2-methyl-6-oxo- 1,6- dihydropyridin-3- yl)-2,3- dihydroquinazolin- 4(1H)-one |
| 243 | 6-chloro-1-(2- ethyl-4- fluorophenyl)-3- (3-methylpyridin- 4-yl)-4-oxo- 1,2,3,4- tetrahydroquin- azoline-7- carbonitrile | 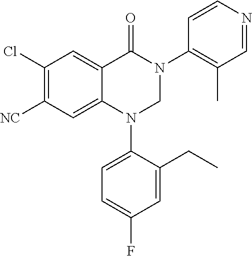 [SMILES: CCc1cc(F)ccc1N1CN(c2ccncc2C)C(=O)c2cc(Cl)c(C#N)cc21] | ¹H NMR (400 MHz, DMSO-d₆) δ: 8.54 (s, 1H), 8.48 (d, J = 4.4 Hz, 1H), 8.06 (s, 1H), 7.40 (dd, J = 5.4, 8.8 Hz, 1H), 7.33-7.29 (m, 2H), 7.20 (dt, J = 2.9, 8.6 Hz, 1H), 6.81 (s, 1H), 5.56 (br s, 1H), 5.05 (br s, 1H), 2.61-2.56 (m, 2H), 2.17 (s, 3H), 1.13 (t, J = 7.3 Hz, 3H). MS (m/z) 421.3 (M + H)⁺ | 6-chloro-1-(2- ethyl-4- fluorophenyl)-7- fluoro-3-(3- methylpyridin-4- yl)-2,3- dihydroquinazolin- 4(1H)-one |

### Example 244

### 4-(6-Chloro-7-(difluoromethyl)-1-(4-fluoro-2-methylphenyl)-4-oxo-1,4-dihydroquinazolin-3(2H)-yl)-3-methylpyridine 1-oxide

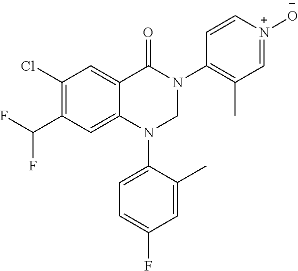 [SMILES: Cc1c[n+]([O-])ccc1N1CN(c2ccc(F)cc2C)c2cc(C(F)F)c(Cl)cc2C1=O]

To a solution of 6-chloro-7-(difluoromethyl)-1-(4-fluoro-2-methylphenyl)-3-(3-methylpyridin-4-yl)-2,3-dihydroquinazolin-4(1H)-one (62.8 mg, 0.145 mmol) in DCM (1 mL) at 0° C. was added mCPBA (50.2 mg, 0.291 mmol). The reaction mixture was stirred at this temperature for 3 hours. The reaction mixture was diluted with DCM, washed with sat. bicarbonate, dried over Na₂SO₄ and concentrated. The crude material was purified by column chromatography (Isco, 24 g column, 0-10% MeOH/DCM) to provide the title compound as a white solid (47.7 mg, 0.104 mmol, 71.8% yield). ¹H NMR (400 MHz, DMSO-d₆) δ: 8.30-8.24 (m, 1H), 8.14 (dd, J=6.85, 1.96 Hz, 1H), 7.96 (s, 1H), 7.43-7.35 (m, 2H), 7.32 (dd, J=9.78, 2.93 Hz, 1H), 7.19 (td, J=8.56, 2.93 Hz, 1H), 7.13 (t, J=53.8 Hz, 1H), 6.49 (s, 1H), 5.76-4.99 (m, 2H), 2.23 (s, 3H), 2.10 (s, 3H). MS (m/z) 448.3 (M+H)⁺.

Examples 245-256 were prepared from the indicated Pyridine by methods analogous to those described for Example 244

| Ex. | Name | Structure | Characterization | Pyridine |
|---|---|---|---|---|
| 245 | 4-(6-chloro-1-(2- ethyl-4- fluorophenyl)-7- fluoro-4-oxo-1,4- dihydroquinazolin- 3(2H)-y1)-3- methylpyridine 1-oxide | 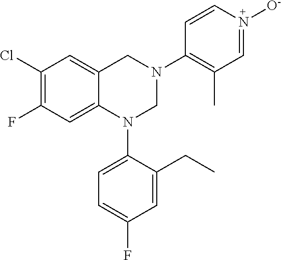 | ¹H NMR (400 MHz, DMSO-d₆) δ: 8.26 (d, J = 1.5 Hz, 1H), 8.13 (dd, J = 2.0, 6.8 Hz, 1H), 7.97 (d, J = 7.8 Hz, 1H), 7.41- 7.30 (m, 3H), 7.23-7.18 (m, 1H), 6.16 (d, J = 10.8 Hz, 1H), 5.50 (br s, 1H), 4.96 (br s, 1H), 2.62-2.56 (m, 2H), 2.11 (s, 3H), 1.14 (t, J = 7.6 Hz, 3H). MS (m/z) 430.3 (M + H)⁺ | 6-chloro-1-(2- ethyl-4- fluorophenyl)-7- fluoro-3-(3- methylpyridin-4- yl)-2,3- dihydro- quinazolin- 4(1H)-one |
| 246 | 4-(6-chloro-7- fluoro-1-(2- methyl-4- (trifluoromethoxy) phenyl)-4-oxo- 1,4- dihydroquinazolin- 3(2H)-yl)-3- methylpyridine 1-oxide | 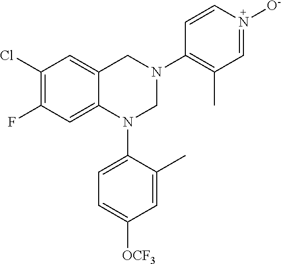 | ¹H NMR (400MHz, DMSO-d₆) δ: 8.25 (d, J = 1.5 Hz, 1H), 8.13 (dd, J = 2.0, 6.8 Hz, 1H), 7.99 (d, J = 8.3 Hz, 1H), 7.46- 7.43 (m, 2H), 7.37-7.33 (m, 2H), 6.31-6.28 (m, 1H), 5.44-5.11 (m, 2H), 2.27 (s, 3H), 2.09 (s, 3H). MS (m/z) 482.2 (M + H)⁺ | 6-chloro-7- fluoro-1-(2- methyl-4- (trifluoromethoxy) phenyl)-3-(3- methylpyridin-4- yl)-2,3- dihydro- quinazolin- 4(1H)-one |
| 247 | 4-(6-chloro-7- cyano-1-(2-ethyl- 4-fluorophenyl)- 4-oxo-1,4- dihydroquinazolin- 3(2H)-yl)-3- methylpyridine 1-oxide | 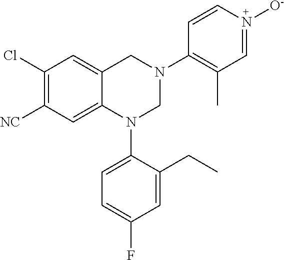 | ¹H NMR (400MHz, DMSO-d₆) δ: 8.27 (s, 1H), 8.14 (dd, J = 2.4, 6.8 Hz, 1H), 8.04 (s, 1H), 7.41-7.30 (m, 3H), 7.23- 7.18 (m, 1H), 6.79 (br s, 1H), 5.50 (br s, 1H), 5.03 (br s, 1H), 2.58 (q, J = 7.3 Hz, 2H), 2.10 (s, 3H), 1.14 (t, J = 7.3 Hz, 3H). MS (m/z) 437.3 (M + H)⁺ | 6-chloro-1-(2- ethyl-4- fluorophenyl)-3- (3-methylpyridin- 4-yl)-4-oxo- 1,2,3,4-tetra- hydroquinazoline- 7-carbonitrile |
| 248 | 2-carbamoyl-5- (6-chloro-5- fluoro-1-(4- fluoro-2- methylphenyl)-4- oxo-1,4- dihydroquinazolin- 3(2H)- yl)pyridine 1- oxide | 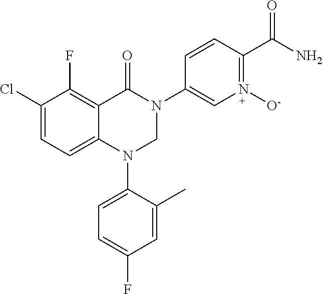 | ¹H NMR (400MHz, DMSO-d₆) δ: 10.15 (br d, J = 4.4 Hz, 1H), 8.62 (d, J = 2.0 Hz, 1H), 8.24- 8.19 (m, 2H), 7.63 (dd, J = 2.0, 8.8 Hz, 1H), 7.55 (dd, J = 7.6, 9.0 Hz, 1H), 7.37 (dd, J = 5.6, 8.6 Hz, 1H), 7.29 (dd, J = 2.9, 9.8 Hz, 1H), 7.17 (dt, J = 2.9, 8.6 Hz, 1H), 6.16 (dd, J = 1.0 Hz, 1H), 5.40 (d, J = 1.0 Hz, 2H), 2.21 (s, 3H). MS (m/z) 445.3 (M + H)⁺ | 5-(6-chloro-5- fluoro-1-(4- fluoro-2- methylphenyl)-4- oxo-1,4- dihydro- quinazolin- 3(2H)- yl)picolinamide |
| 249 | 3-(1-(4-fluoro-2- methylphenyl)-4- oxo-6- (trifluoromethyl)- 1,4- dihydroquinazolin- 3(2H)- yl)pyridine 1- oxide | 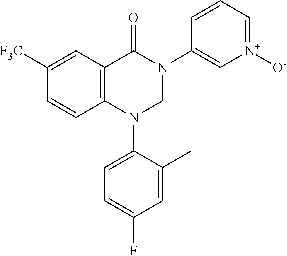 [SMILES: Cc1cc(F)ccc1N1CN(c2ccc[n+]([O-])c2)C(=O)c2cc(C(F)(F)F)ccc21] | ¹H NMR (400 MHz, DMSO-d₆) δ: 8.38-8.47 (m, 1H) 8.10-8.20 (m, 2H) 7.62-7.72 (m, 1H) 7.40-7.50 (m, 3H) 7.30- 7.36 (m, 1H) 7.16-7.26 (m, 1H) 6.36-6.44 (m, 1H) 5.50-5.60 (m, 1H) 5.21-5.34 (m, 1H) 2.17- 2.27 (m, 3H). MS (m/z) 418.3 (M + H)+ | 1-(4-fluoro-2- methylphenyl)-3- (pyridin-3-yl)-6- (trifluoromethyl)- 2,3- dihydro- quinazolin- 4(1H)-one |
| 250 | 3-(1-(4-fluoro-2- methylphenyl)-4- oxo-6- (trifluoromethyl)- 1,4- dihydroquinazolin- 3(2H)-yl)-4- methylpyridine 1-oxide | 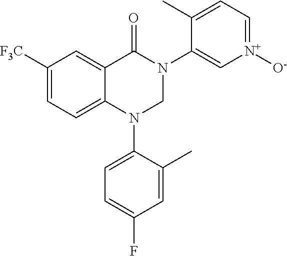 [SMILES: Cc1cc[n+]([O-])cc1N1CN(c2ccc(F)cc2C)c2ccc(C(F)(F)F)cc2C1=O] | ¹H NMR (400 MHz, DMSO-d₆) δ: 8.30-8.42 (m, 1H) 8.06-8.18 (m, 2H) 7.69 (dd, J = 8.80, 2.45 Hz, 1H) 7.29-7.47 (m, 3H) 7.21 (br t, J = 7.09 Hz, 1H) 6.31-6.48 (m, 1H) 4.86-5.70 (m, 2H) 2.14-2.28 (m, 6H) MS (m/z) 432.3 (M + H)⁺ | 1-(4-fluoro-2- methylphenyl)-3- (4-methylpyridin- 3-yl)-6- (trifluoromethyl)- 2,3- dihydro- quinazolin- 4(1H)-one |
| 251 | 5-(1-(4-fluoro-2- methylphenyl)-4- oxo-6- (trifluoromethyl)- 1,4- dihydroquinazolin- 3(2H)- yl)pyridazine 1- oxide | 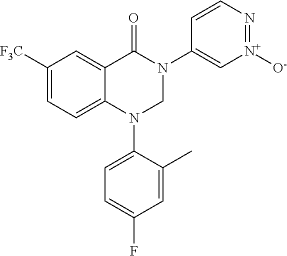 [SMILES: Cc1cc(F)ccc1N1CN(c2ccn[n+]([O-])c2)C(=O)c2cc(C(F)(F)F)ccc21] | ¹H NMR (DMSO-d₆) δ: 8.62-8.69 (m, 1H), 8.50- 8.60 (m, 1H), 8.14-8.21 (m, 1H), 7.67-7.76 (m, 1H), 7.41-7.47 (m, 2H), 7.31-7.36 (m, 1H), 7.19- 7.26 (m, 1H), 6.38-6.44 (m, 1H), 5.54-5.62 (m, 1H), 5.36-5.45 (m, 1H), 2.13-2.24 (m, 3H) MS (m/z) 419.3 (M + H)⁺ | 1-(4-fluoro-2- methylphenyl)-3- (pyridazin-4-yl)-6- (trifluoromethyl)- 2,3- dihydro- quinazolin- 4(1H)-one |
| 252 | 4-(1-(4-fluoro-2- methylphenyl)-4- oxo-6- (trifluoromethyl)- 1,4- dihydroquinazolin- 3(2H)- yl)pyridazine 1- oxide | 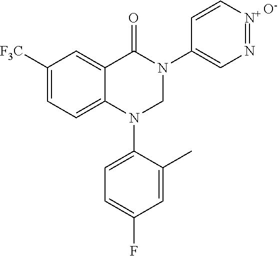 | ¹H NMR (DMSO-d₆) δ: 8.73-8.80 (m, 1H), 8.35- 8.40 (m, 1H), 8.13-8.18 (m, 1H), 7.94-8.02 (m, 1H), 7.66-7.74 (m, 1H), 7.42-7.49 (m, 1H), 7.31- 7.38 (m, 1H), 7.18-7.27 (m, 1H), 6.39 (d, J = 8.8 Hz, 1H), 5.52-5.66 (m, 1H), 5.32-5.41 (m, 1H), 2.22 (s, 3H) MS (m/z) 419.3 (M + H)⁺ | 1-(4-fluoro-2- methylphenyl)-3- (pyridazin-4-yl)-6- (trifluoromethyl)- 2,3- dihydro- quinazolin- 4(1H)-one |
| 253 | 3-(1-(4-fluoro-2- methylphenyl)-4- oxo-6- (trifluoromethyl)- 1,4- dihydroquinazolin- 3(2H)-yl)-2- methylpyridine 1-oxide | 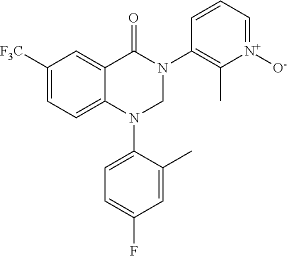 [SMILES: Cc1cc(F)ccc1N1CN(c2ccc[n+]([O-])c2C)C(=O)c2cc(C(F)(F)F)ccc21] | ¹H NMR (400 MHz, CHLOROFORM-d) δ: 8.40 (d, J = 0.98 Hz, 1H) 8.12-8.36 (m, 1H) 7.56 (dd, J = 8.31, 1.96 Hz, 1H) 7.19-7.25 (m, 2H) 7.10- 7.18 (m, 1H) 7.01-7.10 (m, 1H) 6.34-6.48 (m, 1H) 5.44-5.58 (m, 1H) 5.04-5.20 (m, 1H) 4.65- 4.77 (m, 1H) 2.44-2.56 (m, 3H) 2.24-2.36 (m, 3H). MS (m/z) 432.1 (M + H)⁺ | 1-(4-fluoro-2- methylphenyl)-3- (2-methylpyridin- 3-yl)-6- (trifluoromethyl)- 2,3- dihydro- quinazolin- 4(1H)-one |
| 254 | 4-(1-(4-fluoro-2- methylphenyl)-4- oxo-6- (trifluoromethyl)- 1,4- dihydroquinazolin- 3(2H)-yl)-3- methylpyridine 1-oxide | 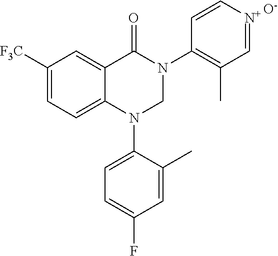 | ¹H NMR (400 MHz, CHLOROFORM-d) δ: 8.39 (d, J = 1.96 Hz, 1H), 8.18 (d, J = 1.47 Hz, 1H), 8.12 (dd, J = 6.60, 1.71 Hz, 1H), 7.56 (dd, J = 8.80, 1.96 Hz, 1H), 7.11-7.22 (m, 3H), 7.05 (td, J = 8.07, 2.93 Hz, 1H), 6.41 (d, J = 8.80 Hz, 1H), 5.34 (br dd, J = 6.85, 4.89 Hz, 1H), 4.89 (br s, 1H), 2.30 (s, 3H), 2.26 (s, 3H). MS (m/z) 432.4 (M + H)⁺ | 1-(4-fluoro-2- methylphenyl)-3- (3-methylpyridin- 4-yl)-6- (trifluoromethyl)- 2,3- dihydro- quinazolin- 4(1H)-one |
| 255 | 3-(1-(4-fluoro-2- methylphenyl)-4- oxo-6- (trifluoromethyl)- 1,4- dihydroquinazolin- 3(2H)-yl)-5- methylpyridine 1-oxide | 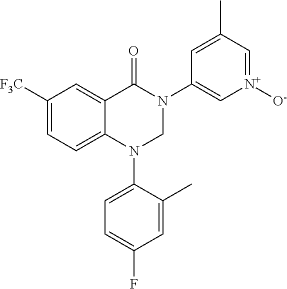 [SMILES: Cc1cc(N2CN(c3ccc(F)cc3C)c3ccc(C(F)(F)F)cc3C2=O)c[n+]([O-])c1] | ¹H NMR (400 MHz, CHLOROFORM-d) δ: 8.31-8.47 (m, 1H) 8.03- 8.21 (m, 1H) 7.90-8.03 (m, 1H) 7.45-7.60 (m, 1H) 7.25-7.28 (m, 1H) 7.16-7.22 (m, 1H) 7.10- 7.14 (m, 1H) 6.98-7.09 (m, 1H) 6.33-6.48 (m, 1H) 5.31-5.40 (m, 1H) 5.01-5.13 (m, 1H) 2.34- 2.39 (m, 3H), 2.26-2.30 (m, 3H). MS (m/z) 432.3 (M + H)⁺ | 1-(4-fluoro-2- methylphenyl)-3- (5-methylpyridin- 3-yl)-6- (trifluoromethyl)- 2,3- dihydroquinazolin- 4(1H)-one |
| 256 | 3-methyl-4-(1-(2- methyl-4- (trifluoromethoxy) phenyl)-4-oxo-6- (trifluoromethyl)- 1,4- dihydroquinazolin- 3(2H)- yl)pyridine 1- oxide | 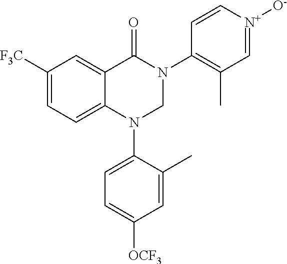 [SMILES: Cc1c[n+]([O-])ccc1N1CN(c2ccc(OC(F)(F)F)cc2C)c2ccc(C(F)(F)F)cc2C1=O] | ¹H-NMR (400 MHz, DMSO-d₆): δ 8.27 (d, J = 1.20 Hz, 1H), 8.15-8.13 (m, 2H), 7.71 (dd, J = 2.00, 8.80 Hz, 1H), 7.50- 7.48 (m, 2H), 7.41-7.34 (m, 2H), 6.43 (d, J = 8.80 Hz, 1H), 5.56 (br s, 1H), 5.10 (br s, 1H), 2.26 (s, 3H), 2.12 (s, 3H). MS (m/z) 498.1 (M + H)⁺ | 1-(2-methyl-4- (trifluoromethoxy) phenyl)-3-(3- methylpyridin-4- yl)-6- (trifluoromethyl)- 2,3- dihydroquinazolin- 4(1H)-one |

### Example 257

### 3-(2-Amino-6-oxo-1,6-dihydropyridin-3-yl)-1-(4-fluoro-2-methylphenyl)-6-(trifluoromethyl)-2,3-dihydroquinazolin-4(1H)-one

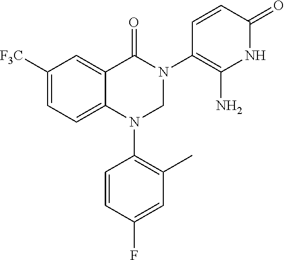 [SMILES: Cc1cc(F)ccc1N1CN(c2ccc(=O)[nH]c2N)C(=O)c2cc(C(F)(F)F)ccc21]

N-(3-(1-(4-fluoro-2-methylphenyl)-4-oxo-6-(trifluoromethyl)-1,4-dihydroquinazolin-3(2H)-yl)-6-oxo-1,6-dihydropyridin-2-yl)acetamide (42.32 mg, 0.089 mmol) was dissolved in 7N ammonia in MeOH (2.00 mL, 14.00 mmol) in a 10 ml microwave reaction vessel. The vessel was sealed and heated to 60° C. The solvent was concentrated and the residue was purified by MDAP (XSELECT CSH C18 (150 mm×30 mm) 5 μm column, A=0.1% v/v solution of formic acid in water, B=0.1% v/v solution of formic acid in acetonitrile, 30-99% B, gradient time 3-17 min) to provide the title compound (28.5 mg, 0.066 mmol, 74% yield). MS (m/z) 489 (M+H)⁺. ¹H NMR (DMSO-d₆, 400 MHz) δ: 10.5-10.7 (m, 1H), 8.07 (s, 1H), 7.61 (dd, 1H, J=2.0, 8.8 Hz), 7.4-7.5 (m, 1H), 7.31 (dd, 1H, J=2.9, 9.8 Hz), 7.1-7.2 (m, 2H), 6.28 (d, 1H, J=8.8 Hz), 5.9-6.0 (m, 2H), 5.50 (br d, 1H, J=7.8 Hz), 5.1-5.3 (m, 1H), 4.7-5.0 (m, 1H), 2.2-2.3 (m, 3H).

### Example 258

### 3-(6-Amino-2-methylpyridin-3-yl)-1-(4-fluoro-2-methylphenyl)-6-(trifluoromethyl)-2,3-dihydroquinazolin-4(1H)-one

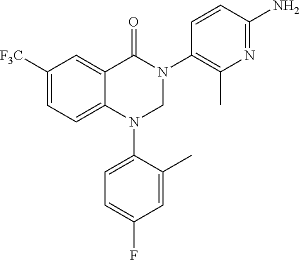 [SMILES: Cc1cc(F)ccc1N1CN(c2ccc(N)nc2C)C(=O)c2cc(C(F)(F)F)ccc21]

A mixture of N-(5-(1-(4-fluoro-2-methylphenyl)-4-oxo-6-(trifluoromethyl)-1,4-dihydroquinazolin-3(2H)-yl)-6-methylpyridin-2-yl)acetamide (186 mg, 0.394 mmol) in methanol (2 mL) was added to sodium methoxide (25% in MeOH, 3 mL, 12.65 mmol) in a 10 ml sealed tube. The tube was heated to 70° C. for 2 hours. Solvent was removed and the crude product was purified by MDAP (XSELECT CSH C18 (150 mm×30 mm) 5 μm column, A=0.1% v/v solution of formic acid in water, B=0.1% v/v solution of formic acid in acetonitrile, 5-35% B, gradient time 3-12 min) to provide the title compound (48 mg, 0.112 mmol, 28.3% yield). ¹H NMR (400 MHz, DMSO-d₆) δ: 8.14-8.05 (m, 1H), 7.65 (dd, J=8.31, 1.96 Hz, 1H), 7.42 (dd, J=8.80, 5.38 Hz, 1H), 7.36-7.25 (m, 2H), 7.20 (br s, 1H), 6.26-6.40 (m, 2H), 6.04 (s, 2H), 5.65-4.65 (m, 2H), 2.24 (s, 3H), 2.18 (s, 3H). MS (m/z) 431.3 (M+H)⁺.

### Example 259

### 2-((5-(6-Chloro-1-(4-fluoro-2-methylphenyl)-4-oxo-1,4-dihydroquinazolin-3(2H)-yl)-6-methylpyridin-2-yl)oxy)acetic Acid

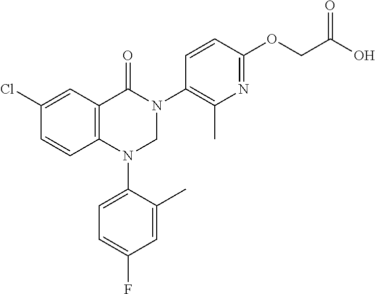 [SMILES: Cc1cc(F)ccc1N1CN(c2ccc(OCC(=O)O)nc2C)C(=O)c2cc(Cl)ccc21]

### Step 1: Ethyl 2-((5-(6-chloro-1-(4-fluoro-2-methylphenyl)-4-oxo-1,4-dihydroquinazolin-3(2H)-yl)-6-methylpyridin-2-yl)oxy)acetate

To a solution of 6-chloro-1-(4-fluoro-2-methylphenyl)-3-(2-methyl-6-oxo-1,6-dihydropyridin-3-yl)-2,3-dihydroquinazolin-4(1H)-one (200 mg, 0.503 mmol) in DMF (3.00 mL) was added sodium hydride (60% wt in mineral oil) (30.2 mg, 0.754 mmol) at 0° C. After 1 h, ethyl 2-bromoacetate (0.100 mL, 0.905 mmol) was added. The reaction mixture was warmed to room temperature and stirred for 3 h. The mixture was quenched with sat. NH₄Cl aqueous solution and extracted with EtOAc (3×). The combined organic layers were washed with brine, dried over anhydrous MgSO₄ and filtered. The filtrate was concentrated in vacuo and was purified by column chromatography (Isco, 40 g RediSep Rf Gold high performance flash columns, 0-100% EtOAc/heptane over 30 mins) to give the title compound as a colorless oil (168 mg, 0.347 mmol, 69% yield). MS (m/z) 484.3 (M+H)⁺.

### Step 2: 2-((5-(6-Chloro-1-(4-fluoro-2-methylphenyl)-4-oxo-1,4-dihydroquinazolin-3(2H)-yl)-6-methylpyridin-2-yl)oxy)acetic Acid

LiOH.H₂O (149 mg, 3.55 mmol) was added to a stirring solution of ethyl 2-((5-(6-chloro-1-(4-fluoro-2-methylphenyl)-4-oxo-1,4-dihydroquinazolin-3(2H)-yl)-6-methylpyridin-2-yl)oxy)acetate (172 mg, 0.355 mmol) in methanol (2.00 mL) and water (2.00 mL) at room temperature. The reaction mixture was stirred for 2 hours. The mixture was acidified using 1 N HCl and extracted with EtOAc (3×). The combined organic layers were washed with brine, dried over anhydrous MgSO₄ and filtered. The filtrate was concentrated in vacuo to give the title compound as a white solid (153 mg, 0.336 mmol, 94% yield). ¹H NMR (400 MHz, DMSO-d₆) δ: 12.83 (br s, 1H), 7.80 (d, J=2.9 Hz, 1H), 7.66 (d, J=1.0 Hz, 1H), 7.39 (dd, J=2.4, 8.8 Hz, 1H), 7.32-7.27 (m, 2H), 7.15 (t, J=1.0 Hz, 1H), 6.79 (d, J=8.8 Hz, 1H), 6.24 (br s, 1H), 5.52-5.13 (m, 2H), 4.80 (s, 2H), 2.25-2.23 (m, 6H). MS (m/z) 456.3 (M+H)⁺.

### Example 260

### 1-(4-Fluoro-2-methylphenyl)-3-(2-methoxy-6-oxo-1,6-dihydropyridin-3-yl)-7-(trifluoromethyl)-2,3-dihydroquinazolin-4(1H)-one

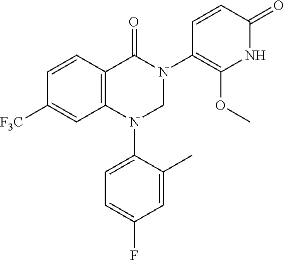 [SMILES: COc1[nH]c(=O)ccc1N1CN(c2ccc(F)cc2C)c2cc(C(F)(F)F)ccc2C1=O]

To a solution of 1-(4-fluoro-2-methylphenyl)-3-(2-methoxy-6-((tetrahydro-2H-pyran-2-yl)oxy)pyridin-3-yl)-7-(trifluoromethyl)-2,3-dihydroquinazolin-4(1H)-one (144 mg, 0.271 mmol) was added HCl (4 N in 1,4-dioxane, 0.50 mL, 2.000 mmol). The reaction was stirred at room temperature for 1 h and then concentrated in vacuo. The crude product was purified by MDAP (XSELECT CSH C18 (150 mm×30 mm) 5 μm column, A=0.1% v/v solution of formic acid in water, B=0.1% v/v solution of formic acid in acetonitrile, 50-99% B, 17 min overall run time) to give the title compound as a white solid (90.3 mg, 0.202 mmol, 74.5% yield). ¹H NMR (400 MHz, DMSO-d₆) δ: 10.84 (br. s., 1H), 8.06 (d, J=8.07 Hz, 1H), 7.58 (d, J=8.31 Hz, 1H), 7.39 (dd, J=8.68, 5.50 Hz, 1H), 7.31 (dd, J=9.66, 2.81 Hz, 1H), 7.24-7.16 (m, 2H), 6.40 (s, 1H), 6.24 (d, J=8.07 Hz, 1H), 5.30 (br. s., 1H), 4.82 (br. s., 1H), 3.76 (s, 3H), 2.25 (s, 3H). MS (m/z) 448.3 (M+H)⁺.

### Example 261

### 3-(6-Aminopyridin-3-yl)-1-(4-fluoro-2-methylphenyl)-7-(trifluoromethyl)-2,3-dihydroquinazolin-4(1H)-one

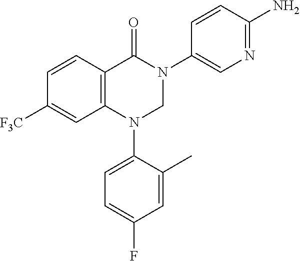 [SMILES: Cc1cc(F)ccc1N1CN(c2ccc(N)nc2)C(=O)c2ccc(C(F)(F)F)cc21]

To a solution of 1-(4-fluoro-2-methylphenyl)-3-(6-nitropyridin-3-yl)-7-(trifluoromethyl)-2,3-dihydroquinazolin-4(1H)-one (49.5 mg, 0.111 mmol) in methanol (1.50 mL) was added Pd/C (10% wt) (5.90 mg, 0.055 mmol). The reaction was stirred under hydrogen (1 atm, balloon) for 20 h. The reaction mixture was filtered through a Celite pad and washed with copious MeOH. The filtrate was concentrated and the crude product was purified by MDAP (XSELECT CSH C18 (150 mm×30 mm) 5 μm column, A=0.1% v/v solution of formic acid in water, B=0.1% v/v solution of formic acid in acetonitrile, 15-55% B, gradient time 1-10.5 min) to give the title compound as an off-white solid (25.9 mg, 0.062 mmol, 56% yield). ¹H NMR (400 MHz, DMSO-d₆) δ: 8.07 (d, J=8.07 Hz, 1H) 7.92 (d, J=2.69 Hz, 1H) 7.42-7.38 (m, 2H) 7.31 (dd, J=9.78, 2.93 Hz, 1H), 7.25-7.16 (m, 2H), 6.48 (d, J=8.80 Hz, 1H), 6.39 (s, 1H), 6.13 (br. s., 2H), 5.39 (br. s., 1H), 5.04 (br. s., 1H), 2.23 (s, 3H). MS (m/z) 417.3 (M+H)⁺.

### Example 262

### 4-(1-(4-Fluoro-2-methylphenyl)-4-oxo-7-(trifluoromethyl)-1,4-dihydroquinazolin-3(2H)-yl)benzoic Acid

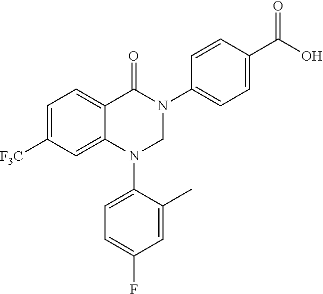 [SMILES: Cc1cc(F)ccc1N1CN(c2ccc(C(=O)O)cc2)C(=O)c2ccc(C(F)(F)F)cc21]

A suspension of methyl 4-(1-(4-fluoro-2-methylphenyl)-4-oxo-7-(trifluoromethyl)-1,4-dihydroquinazolin-3(2H)-yl)benzoate (0.13 g, 0.284 mmol) and LiOH (0.034 g, 1.418 mmol) in THF (2.000 ml) and water (2 ml) was stirred at RT overnight. Solvent was removed and the resulted water slurry was washed with Et₂O and acidified with 6N HCl until pH˜2. The solid precipitate was collected by filtration, washed with water, and air dried to give the title compound as a white solid (90 mg, 0.203 mmol, 71.4% yield). ¹H NMR (400 MHz, DMSO-d₆) δ: 8.14 (d, J=8.1 Hz, 1H), 7.93 (dt, J=8.6, 1.8 Hz, 2H), 7.4-7.5 (m, 3H), 7.33 (dd, J=9.6, 3.0 Hz, 1H), 7.28 (dd, J=8.4, 1.3 Hz, 1H), 7.21 (td, J=8.6, 3.3 Hz, 1H), 6.4-6.5 (m, 1H), 5.53 (br s, 1H), 5.22 (br s, 1H), 2.23 (s, 2H). MS (m/z) 445.2 (M+H)⁺.

Example 263 was prepared from the indicated intermediate by methods analogous to those described for Example 262.

| Ex. | Name | Structure | Characterization | Intermediate |
|---|---|---|---|---|
| 263 | 3-(1-(4-fluoro-2- methylphenyl)-4- oxo-7- (trifluoromethyl)- 1,4- dihydroquinazolin- 3(2H)- yl)benzoic acid | 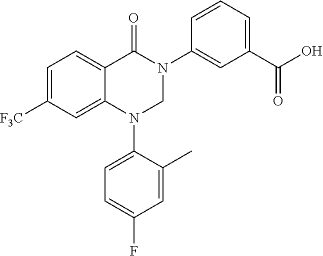 [SMILES: Cc1cc(F)ccc1N1CN(c2cccc(C(=O)O)c2)C(=O)c2ccc(C(F)(F)F)cc21] | ¹H NMR (400 MHz, DMSO-d₆) δ: 8.14 (d, J = 7.9 Hz, 1H), 7.94 (t, J = 1.8 Hz, 1H), 7.84 (dt, J = 7.6, 1.3 Hz, 1H), 7.6- 7.7 (m, 1H), 7.52 (t, J = 7.6 Hz, 1H), 7.44 (dd, J = 8.6, 5.3 Hz, 1H), 7.32 (dd, J = 9.9, 3.0 Hz, 1H), 7.28 (dd, J = 8.4, 1.3 Hz, 1H), 7.20 (td, J = 8.6, 3.0 Hz, 1H), 6.4-6.5 (m, 1H), 5.57 (br s, 1H), 5.24 (br s, 1H), 2.24 (s, 3H) MS (m/z) 445.2 (M + H)⁺ | methyl 3-(1-(4- fluoro-2- methylphenyl)-4- oxo-7- (trifluoromethyl)- 1,4- dihydroquinazolin- 3(2H)- yl)benzoate |

### Example 264

### 1-(4-Fluoro-2-methylphenyl)-3-(2-hydroxy-6-oxo-1,6-dihydropyridin-3-yl)-7-(trifluoromethyl)-2,3-dihydroquinazolin-4(1H)-one

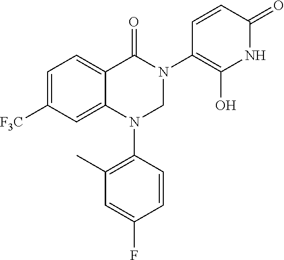 [SMILES: Cc1cc(F)ccc1N1CN(c2ccc(=O)[nH]c2O)C(=O)c2ccc(C(F)(F)F)cc21]

### Step 1: 3-(2-Bromo-6-oxo-1,6-dihydropyridin-3-yl)-1-(4-fluoro-2-methylphenyl)-7-(trifluoromethyl)-2,3-dihydroquinazolin-4(1H)-one

This compound was prepared from 3-(2-bromo-6-methoxypyridin-3-yl)-1-(4-fluoro-2-methylphenyl)-7-(trifluoromethyl)-2,3-dihydroquinazolin-4(1H)-one by methods analogous to those described for Example 26. MS (m/z) 498.1 (M+3H)⁺

### Step 2: 1-(4-Fluoro-2-methylphenyl)-3-(2-hydroxy-6-oxo-1,6-dihydropyridin-3-yl)-7-(trifluoromethyl)-2,3-dihydroquinazolin-4(1H)-one

A mixture of 3-(2-bromo-6-oxo-1,6-dihydropyridin-3-yl)-1-(4-fluoro-2-methylphenyl)-7-(trifluoromethyl)-2,3-dihydroquinazolin-4(1H)-one (0.070 g, 0.141 mmol) in 1,4-dioxane (0.705 ml) was purged with N₂ for 15 minutes before Pd₂(dba)₃ (6.46 mg, 7.05 μmol), tBuXphos (0.012 g, 0.028 mmol) and KOH (0.024 g, 0.423 mmol) (in 0.705 mL of water) were added. The reaction was purged with N₂ for 15 minutes, sealed and stirred at 100° C. overnight. The reaction was cooled, acidified to pH˜2 with 1N HCl and extracted with EtOAc. The organic layer was concentrated and purified by MDAP (XSelect CSH Prep C18 Sum OBD column, 30-85% gradient, acetonitrile with 0.1% formic acid/water with 0.1% formic acid, 40 mL/min flow rate, 17 min run time) to give the title compound as a beige solid (12 mg, 0.028 mmol, 19.63% yield). MS (m/z) 434.3 (M+H)⁺. ¹H NMR (400 MHz, DMSO-d₆) δ: 12.10-11.10 (m, 2H), 8.05 (d, J=7.8 Hz, 1H), 7.48-7.38 (m, 2H), 7.31 (dd, J=2.9, 9.8 Hz, 1H), 7.24-7.13 (m, 2H), 6.34 (s, 1H), 5.60 (br s, 1H), 5.31 (br s, 1H), 4.78 (br s, 1H), 2.24 (s, 3H).

### Example 265

### 1-(4-Fluoro-2-methylphenyl)-3-(2-methyl-6-oxo-1,6-dihydropyridin-3-yl)-7-(oxetan-3-yl)-2,3-dihydroquinazolin-4(1H)-one

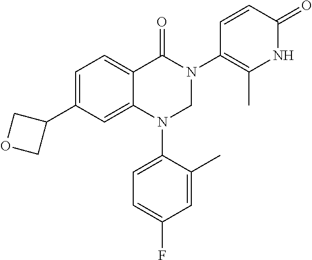 [SMILES: Cc1cc(F)ccc1N1CN(c2ccc(=O)[nH]c2C)C(=O)c2ccc(C3COC3)cc21]

TMS-Cl (0.044 ml, 0.346 mmol) was added to a mixture of 1-(4-fluoro-2-methylphenyl)-3-(6-methoxy-2-methylpyridin-3-yl)-7-(oxetan-3-yl)-2,3-dihydroquinazolin-4(1H)-one (0.025 g, 0.058 mmol) and sodium iodide (0.052 g, 0.346 mmol) in acetonitrile (1 ml) under N₂ and the reaction was stirred at 80° C. for 1 hr. The reaction was cooled, treated with MeOH (3 mL) and stirred for 30 minutes. Solvent was removed and the residue was rinsed with water and dried under a stream of N₂. The resultant brown solid was dissolved in DMF (˜1.5 mL) and treated with NaH (excess). After stirring for 1 hr, the reaction was cooled in an ice bath and quenched with NH₄Cl (2 mL). The reaction was extracted with EtOAc (2×) and DCM. The combined organic extracts were concentrated and the residue was purified by column chromatography (Isco, 12 g column, MeOH/DCM) to give the title compound as an off-white solid (14 mg, 0.033 mmol, 57.9% yield). ¹H NMR (400 MHz, METHANOL-d₄) δ: 7.95 (d, J=8.3 Hz, 1H), 7.53 (d, J=9.8 Hz, 1H), 7.29 (dd, J=5.4, 8.8 Hz, 1H), 7.18 (dd, J=2.7, 9.5 Hz, 1H), 7.14-7.03 (m, 2H), 6.48-6.32 (m, 2H), 5.41-5.34 (m, 2H), 4.30 (s, 2H), 3.33 (td, J=1.5, 3.3 Hz, 2H), 2.33 (s, 3H), 2.26 (br s, 3H). MS (m/z) 420.4 (M+H)⁺.

### Example 266

### 1-(4-Fluoro-2-methylphenyl)-3-(4-hydroxy-2-methylphenyl)-6-(trifluoromethyl)-2,3-dihydroquinazolin-4(1H)-one

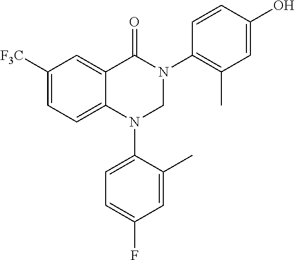 [SMILES: Cc1cc(O)ccc1N1CN(c2ccc(F)cc2C)c2ccc(C(F)(F)F)cc2C1=O]

A mixture of 3-(4-((tert-butyldimethylsilyl)oxy)-2-methylphenyl)-1-(4-fluoro-2-methylphenyl)-6-(trifluoromethyl)-2,3-dihydroquinazolin-4(1H)-one (11 mg, 0.020 mmol) and HCl (15.15 μl, 0.061 mmol, 4N in dioxane) was heated for 18 hr at 80° C., then raised to 95° C. for 36 hr. The residue was purified via Isco Combiflash Rf silica gel chromatography (25% to 100% in EtOAc in heptane; 24 g REDI Sep column over 15 min). The product was further purified via MDAP (XSELECT CSH C18 column (150×30) mm 5 μm, 50% to 90% in 0.1% v/v solution of TFA in water to 0.1% v/v solution of TFA in acetonitrile at ambient temperature over 15 min gradient). The pure fractions were collected, extracted into EtOAc, washed with sat. NaHCO₃ and concentrated to give title compound as a white solid (8.3 mg, 0.019 mmol, 95% yield). MS (m/z) 431.3 (M+H)⁺. ¹H NMR (400 MHz, CHLOROFORM-d) δ: 8.41 (d, J=1.96 Hz, 1H), 7.52 (dd, J=8.80, 1.96 Hz, 2H), 7.21-7.15 (m, 1H), 7.10 (dd, J=9.05, 2.69 Hz, 1H), 7.04-6.97 (m, 1H), 6.57 (br d, J=7.83 Hz, 2H), 6.43-6.30 (m, 1H), 5.46 (br d, J=9.78 Hz, 1H), 5.14-4.99 (m, 1H), 4.69 (br d, J=9.78 Hz, 1H), 2.30 (br s, 3H), 2.20-2.26 (m, 3H).

### Example 267

### 1-(4-Fluoro-2-methylphenyl)-3-(4-hydroxy-2-methylphenyl)-7-(trifluoromethyl)-2,3-dihydroquinazolin-4(1H)-one

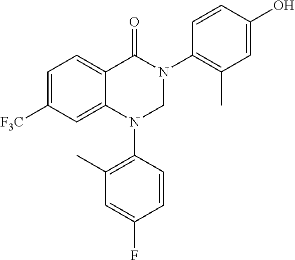 [SMILES: Cc1cc(O)ccc1N1CN(c2ccc(F)cc2C)c2cc(C(F)(F)F)ccc2C1=O]

A solution of 3-(4-((tert-butyldimethylsilyl)oxy)-2-methylphenyl)-1-(4-fluoro-2-methylphenyl)-7-(trifluoromethyl)-2,3-dihydroquinazolin-4(1H)-one (42 mg, 0.077 mmol) and TBAF (231 μl, 0.231 mmol) in dichloromethane (771 μl) was stirred for 2 hr at 25° C. The material was evaporated to dryness and purified via column chromatography (Isco Combiflash Rf, 40 g REDI Sep column, 25% to 100% EtOAc in heptane over 15 min gradient) to give the title compound as a white solid (25 mg, 0.058 mmol, 75% yield). MS (m/z) 431.3 (M+H)⁺. ¹H NMR (400 MHz, DMSO-d₆) δ: 9.50 (s, 1H), 8.08 (d, J=8.31 Hz, 1H), 7.41 (br d, J=3.42 Hz, 1H), 7.32 (dd, J=9.54, 2.69 Hz, 1H), 7.25 (d, J=7.34 Hz, 1H), 7.19 (br s, 1H), 7.08 (d, J=8.31 Hz, 1H), 6.69 (br s, 1H), 6.64 (dd, J=8.31, 2.93 Hz, 1H), 6.46-6.30 (m, 1H), 5.59 (br s, 0.5H), 5.20-5.00 (m, 1H), 4.67 (br s, 0.5H), 2.24 (br s, 3H), 2.12 (s, 3H).

### Example 268

### 1-(4-Fluorophenyl)-3-(6-oxo-1,6-dihydropyridin-3-yl)-7-(trifluoromethyl)-2,3-dihydroquinazolin-4(1H)-one

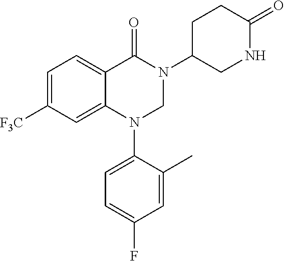 [SMILES: Cc1cc(F)ccc1N1CN(C2CCC(=O)NC2)C(=O)c2ccc(C(F)(F)F)cc21]

A solution of 1-(4-fluoro-2-methylphenyl)-3-(6-oxo-1,6-dihydropyridin-3-yl)-7-(trifluoromethyl)-2,3-dihydroquinazolin-4(1H)-one (20 mg, 0.048 mmol) in ethanol (10 mL) was added to Pt/C (5%, 26 mg) and the resulting mixture was hydrogenated at RT and atmospheric pressure overnight. The catalyst was filtered off and solvent was removed under reduced pressure. The residue was dissolved DMSO (0.9 mL) and purified using high pH MDAP purification system to afford the title compound (10.6 mg, 0.025 mmol, 52.5% yield). MS (m/z) 422.2 (M+H)⁺. ¹H NMR (600 MHz, DMSO-d₆) δ: 8.05 (d, J=8.1 Hz, 1H), 7.41-7.27 (m, 2H), 7.24-7.12 (m, 2H), 6.33 (s, 1H), 5.20-4.77 (m, 2H), 4.70 (br s, 1H), 3.22-3.09 (m, 2H), 2.25 (br s, 2H), 2.23-2.17 (m, 4H), 1.78 (br d, J=2.6 Hz, 1H).

Examples 269-271 were prepared from the indicated intermediate by methods analogous to those described for Example 268.

| Ex. | Name | Structure | Characterization | Intermediate |
|---|---|---|---|---|
| 269 | 1-(4-fluoro-2- methylphenyl)-3- (2-methyl-6-oxo- 1,4,5,6- tetrahydropyridin- 3-yl)-7- (trifluoromethyl)- 2,3- dihydroquinazolin- 4(1H)-one | 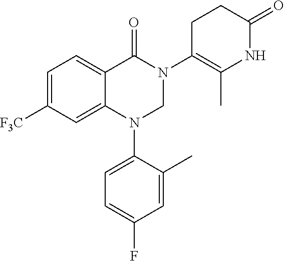 [SMILES: CC1=C(N2CN(c3ccc(F)cc3C)c3cc(C(F)(F)F)ccc3C2=O)CCC(=O)N1] | ¹H NMR (600 MHz, METHANOL-d₄) δ: 8.12 (d, J = 8.1 Hz, 1H), 7.32 (br dd, J = 8.1, 5.1 Hz, 1H), 7.24-7.19 (m, 2H), 7.12 (td, J = 8.4, 2.9 Hz, 1H), 6.45 (s, 1H), 5.51- 4.65 (m, 2H), 2.71-2.43 (m, 4H), 2.29 (s, 3H), 1.83 (br s, 3H). MS (m/z) 434.0 (M + H)⁺ | 1-(4-fluoro-2- methylphenyl)-3- (2-methyl-6-oxo- 1,6- dihydropyridin-3- yl)-7- (trifluoromethyl)- 2,3- dihydroquinazolin- 4(1H)-one |
| 270 | 1-(4-fluoro-2- methylphenyl)-3- (2- oxohexahydro- pyrimidin-5-yl)-7- (trifluoromethyl)- 2,3- dihydroquinazolin- 4(1H)-one | 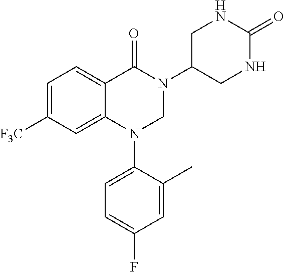 [SMILES: Cc1cc(F)ccc1N1CN(C2CNC(=O)NC2)C(=O)c2ccc(C(F)(F)F)cc21] | ¹H NMR (600 MHz, CHLOROFORM-d) δ: 8.16 (d, J = 8.1 Hz, 1H), 7.18 (d, J = 8.1 Hz, 1H), 7.13-7.08 (m, 2H), 7.04- 6.99 (m, 1H), 6.50 (s, 1H), 5.11-4.98 (m, 4H), 4.98-4.87 (m, 1H), 3.83- 3.58 (m, 2H), 3.52 (br d, J = 10.6 Hz, 1H), 3.48- 3.41 (m, 1H), 2.26 (s, 3H). MS (m/z) 423.0 (M + H)⁺ | 1-(4-fluoro-2- methylphenyl)-3- (2-oxo-1,2- dihydropyrimidin- 5-yl)-7- (trifluoromethyl)- 2,3- dihydroquinazolin- 4(1H)-one |
| 271 | 1-(4-fluoro-2- methylphenyl)-3- (6-methyl-2-oxo- 1,2,3,4- tetrahydro- pyrimidin-5-yl)-7- (trifluoromethyl)- 2,3- dihydroquinazolin- 4(1H)-one | 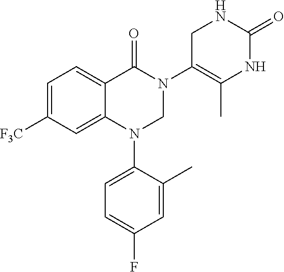 [SMILES: CC1=C(N2CN(c3ccc(F)cc3C)c3cc(C(F)(F)F)ccc3C2=O)CNC(=O)N1] | ¹H NMR (700 MHz, CHLOROFORM-d) δ: 8.16 (d, J = 8.1 Hz, 1H), 7.19 (d, J = 8.1 Hz, 1H), 7.12 (br dd, J = 8.7, 2.3 Hz, 2H), 7.03 (td, J = 8.2, 2.8 Hz, 1H), 6.69 (br s, 1H), 6.49 (s, 1H), 5.22 (br s, 1H), 5.17-4.98 (m, 1H), 4.81-4.62 (m, 1H), 4.34 (br s, 1H), 3.87 (br d, J = 14.4 Hz, 1H), 2.27 (br s, 3H), 1.83 (br s, 3H). MS (m/z) 435.0 (M + H)⁺ | 1-(4-fluoro-2- methylphenyl)-3- (6-methyl-2-oxo- 1,2- dihydropyrimidin- 5-yl)-7- (trifluoromethyl)- 2,3- dihydroquinazolin- 4(1H)-one |

### Example 272

### 3-(1-(4-Fluoro-2-methylphenyl)-4-oxo-7-(trifluoromethyl)-1,4-dihydroquinazolin-3(2H)-yl)benzamide

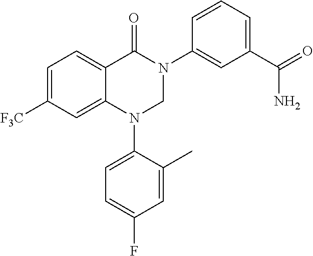 [SMILES: Cc1cc(F)ccc1N1CN(c2cccc(C(N)=O)c2)C(=O)c2ccc(C(F)(F)F)cc21]

To a stirring solution of 3-(1-(4-fluoro-2-methylphenyl)-4-oxo-7-(trifluoromethyl)-1,4-dihydroquinazolin-3(2H)-yl)benzoic acid (350 mg, 0.788 mmol) in DMF (5 mL) were added DIPEA (0.481 mL, 2.76 mmol), HATU (359 mg, 0.945 mmol) and ammonium acetate (304 mg, 3.94 mmol) at 30° C. under nitrogen and the reaction was stirred at 30° C. for 16 hours. The reaction mixture was quenched with ice water (15 mL) and extracted with EtOAc (2×50 mL). The combined organic phases were washed with ice cold water (15 mL), saturated brine (20 mL), dried over Na₂SO₄ and evaporated in vacuo to give the crude product. The residue was purified by column chromatography (Biotage, 25 g column, 50% EtOAc/petroleum ether over 40 minutes) to give the title compound as an off-white solid. The obtained solid was purified again by prep-HPLC (X-Bridge C18, 19×150 mM, 5 micron column, MeCN/(0.1% formic acid in water) to give the title compound as an off white solid (133 mg, 0.297 mmol, 38% yield). ¹H NMR (DMSO-d₆, 400 MHz): b: =8.13 (d, J=7.9 Hz, 1H), 8.03 (br s, 1H), 7.84 (br s, 1H), 7.77 (d, J=7.3 Hz, 1H), 7.59 (d, J=7.8 Hz, 1H), 7.54-7.39 (m, 3H), 7.37-7.25 (m, 2H), 7.24-7.16 (m, 1H), 6.42 (s, 1H), 5.56 (br s, 1H), 5.22 (br s, 1H), 2.24 (s, 3H). MS (m/z) 444.0 (M+H)⁺.

### Example 273

### 3-(6-chloro-1-(4-fluoro-2-methylphenyl)-4-oxo-1,4-dihydroquinazolin-3(2H)-yl)furan-2-carboxamide

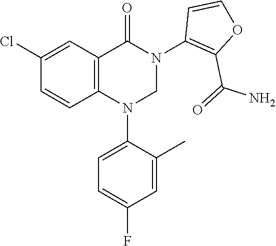 [SMILES: Cc1cc(F)ccc1N1CN(c2ccoc2C(N)=O)C(=O)c2cc(Cl)ccc21]

A sealed tube was charged with methyl 3-(6-chloro-1-(4-fluoro-2-methylphenyl)-4-oxo-1,4-dihydroquinazolin-3(2H)-yl)furan-2-carboxylate (450 mg, 1.085 mmol) in DMF (10 mL) and stirred under nitrogen at 0° C. Aqueous ammonia (4.5 mL, 25.3 mmol) was added in one charge. The reaction mixture was stirred at 80° C. for 20 h. After 20 h reaction mixture was cooled to RT and quenched with ice-cold water (200 ml) and then EtOAc (200 mL) was added. The two layers were separated. The aqueous layer was extracted with EtOAc (100 mL×2). The combined organic layers were dried over sodium sulphate and concentrated under reduced pressure. The crude product was purified by prep-HPLC (Grace reveleris ×2, 12 g Grace C18, 20-85% gradient, acetonitrile with 0.1% formic acid/water with 0.1% formic acid, 20 ml/min flow rate, 50 min overall run time) to give a white solid, that was impure. The solid was further purified by column chromatography (Grace, 25 g SNAP column, 100% EtOAc over 40 min) to give the title compound as a white solid (96 mg, 0.235 mmol, 21.69% yield). ¹H NMR (400 MHz, DMSO-d₆) δ: 7.82-7.76 (m, 2H), 7.754 (s, 1H), 7.500 (s, 1H), 7.40-7.32 (m, 2H), 7.26 (dd, J=2.80, 9.80 Hz, 1H), 7.14 (dt, J=2.80, 12.27 Hz, 1H), 6.82 (d, J=2.00 Hz, 1H), 6.22 (d, J=8.80 Hz, 1H), 5.45-4.35 (m, 2H), 2.21 (s, 3H). MS (m/z) 400.2 (M+H)⁺.

### Example 274

### 4-Methyl-3-(3-(2-methyl-6-oxo-1,6-dihydropyridin-3-yl)-4-oxo-6-(trifluoromethyl)-3,4-dihydroquinazolin-1(2H)-yl)benzamide

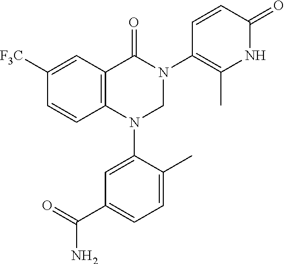 [SMILES: Cc1ccc(C(N)=O)cc1N1CN(c2ccc(=O)[nH]c2C)C(=O)c2cc(C(F)(F)F)ccc21]

### Step 1: 4-Methyl-3-(3-(2-methyl-6-oxo-1,6-dihydropyridin-3-yl)-4-oxo-6-(trifluoromethyl)-3,4-dihydroquinazolin-1(2H)-yl)benzonitrile

This compound was prepared by methods analogous to those described for Example 35 using 3-(3-(6-methoxy-2-methylpyridin-3-yl)-4-oxo-6-(trifluoromethyl)-3,4-dihydroquinazolin-1(2H)-yl)-4-methylbenzonitrile in place of 1-(4-fluoro-2-methylphenyl)-3-(6-methoxy-2-methylpyridin-3-yl)-7-(trifluoromethyl)-2,3-dihydroquinazolin-4(1H)-one. MS (m/z) 439.2 (M+H)⁺.

### Step 2: 4-Methyl-3-(3-(2-methyl-6-oxo-1,6-dihydropyridin-3-yl)-4-oxo-6-(trifluoromethyl)-3,4-dihydroquinazolin-1(2H)-yl)benzamide

To a solution of 4-methyl-3-(3-(2-methyl-6-oxo-1,6-dihydropyridin-3-yl)-4-oxo-6-(trifluoromethyl)-3,4-dihydroquinazolin-1(2H)-yl)benzonitrile (100 mg, 0.228 mmol) in ethanol (20 mL) and water (7 mL) under nitrogen at room temperature was added KOH (38.4 mg, 0.684 mmol). The reaction mixture was stirred at 80° C. for 4 h. The reaction mixture was concentrated under reduced pressure. The residue was partitioned between EtOAc (50 mL) and water (50 mL). The organic layer was washed with sodium bicarbonate (50 mL), dried over sodium sulphate and concentrated under reduced pressure. The crude product was purified by prep-HPLC (X-Bridge C18(10×150 mm) 5 μm, 20% acetonitrile with 0.1% formic acid/80% water with 0.1% formic acid, 8 ml/min flow rate, 17 min overall run time) to give a white solid (20 mg, 0.044 mmol, 19.15% yield). ¹H NMR (400 MHz, DMSO-d₆) δ: 11.79 (s, 1H), 8.10 (d, J=1.60 Hz, 1H), 7.98 (s, 1H), 7.87-7.80 (m, 2H), 7.66 (dd, J=2.00, 8.80 Hz, 1H), 7.52 (d, J=8.00 Hz, 1H), 7.45-7.37 (m, 2H), 6.43-6.31 (m, 1H), 6.20 (d, J=8.40 Hz, 1H), 5.60-4.82 (m, 2H), 2.25-2.27 (m, 3H), 2.11 (s, 3H). MS (m/z) 457.2 (M+H)⁺.

### Example 275

### 4-(6-Chloro-1-(4-fluoro-2-methylphenyl)-4-oxo-1,4-dihydroquinazolin-3(2H)-yl)furan-2-carboxamide

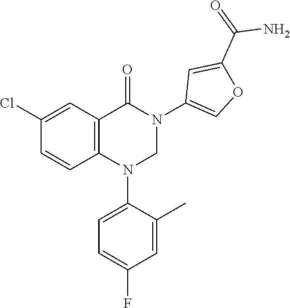 [SMILES: Cc1cc(F)ccc1N1CN(c2coc(C(N)=O)c2)C(=O)c2cc(Cl)ccc21]

### Step 1: 4-(6-Chloro-1-(4-fluoro-2-methylphenyl)-4-oxo-1,4-dihydroquinazolin-3(2H)-yl)furan-2-carboxylic acid

This compound was prepared by methods analogous to those described for Intermediate 216 using methyl 4-(6-chloro-1-(4-fluoro-2-methylphenyl)-4-oxo-1,4-dihydroquinazolin-3(2H)-yl)furan-2-carboxylate in place of ethyl 2-((4-fluoro-2-methylphenyl)amino)-4-(trifluoromethyl)benzoate. MS (m/z) 401.0 (M+H)⁺.

### Step 2: 4-(6-Chloro-1-(4-fluoro-2-methylphenyl)-4-oxo-1,4-dihydroquinazolin-3(2H)-yl)furan-2-carboxamide

This compound was prepared by methods analogous to those described for Intermediate 359 using 4-(6-chloro-1-(4-fluoro-2-methylphenyl)-4-oxo-1,4-dihydroquinazolin-3(2H)-yl)furan-2-carboxylic acid in place of 2-((4-fluoro-2-methylphenyl)amino)-4-(trifluoromethyl)benzoic acid. ¹H NMR (400 MHz, DMSO-d₆) δ: 8.26 (s, 1H), 7.88-7.75 (m, 2H), 7.50 (s, 1H), 7.46-7.37 (m, 3H), 7.32 (dd, J=2.80, 10.00 Hz, 1H), 7.20 (dt, J=2.80, 12.27 Hz, 1H), 6.25 (d, J=8.80 Hz, 1H), 5.32 (br s, 2H), 2.20 (s, 3H). MS (m/z) 400.0 (M+H)⁺.

### Example 276

### 3-(1-(4-Fluoro-2-methylphenyl)-4-oxo-7-(trifluoromethyl)-1,4-dihydroquinazolin-3(2H)-yl)benzenesulfonamide

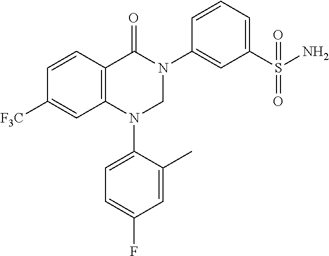 [SMILES: Cc1cc(F)ccc1N1CN(c2cccc(S(N)(=O)=O)c2)C(=O)c2ccc(C(F)(F)F)cc21]

### Step 1: Methyl 3-((3-(1-(4-fluoro-2-methylphenyl)-4-oxo-7-(trifluoromethyl)-1,4-dihydroquinazolin-3(2H)-yl)phenyl)sulfonyl)propanoate

To a solution of 3-(3-bromophenyl)-1-(4-fluoro-2-methylphenyl)-7-(trifluoromethyl)-2,3-dihydroquinazolin-4(1H)-one (900 mg, 1.878 mmol) in DMSO (1 mL) and stirred under nitrogen at room temperature was added sodium 3-methoxy-3-oxopropane-1-sulfinate (981 mg, 5.63 mmol) in one charge followed by copper(I) iodide (1073 mg, 5.63 mmol). The reaction mixture was stirred at 120° C. for 12 hours. The reaction was cooled to room temperature and filtered through Celite. The filtrate was dissolved in ethyl acetate (50 ml), washed with cool water (100 mL), dried over sodium sulphate and evaporated under reduced pressure. The residue was purified by column chromatography (Biotage, 40 g SNAP column, 6-8% EtOAc/petroleum ether over 40 minutes) to give the title compound as a colorless oil (390 mg, 0.588 mmol, 31% yield). MS (m/z) 551 (M+H)⁺.

### Step 2: 3-(1-(4-Fluoro-2-methylphenyl)-4-oxo-7-(trifluoromethyl)-1,4-dihydroquinazolin-3(2H)-yl)benzenesulfonamide

To a solution of methyl 3-((3-(1-(4-fluoro-2-methylphenyl)-4-oxo-7-(trifluoromethyl)-1,4-dihydroquinazolin-3(2H)-yl)phenyl)sulfonyl)propanoate (340 mg, 0.618 mmol) in DMSO (3 mL) and stirred under nitrogen was added sodium methoxide (133 mg, 0.618 mmol) at room temperature under nitrogen. After 15 mins (aminooxy)sulfonic acid (349 mg, 3.09 mmol) and a solution of sodium acetate (177 mg, 2.162 mmol) in 3 ml water were added to the reaction mixture at room temperature. The resulting reaction mixture was stirred at room temperature for 20 hours. The reaction was filtered through a Celite pad and the filtrate was dissolved in ethyl acetate (50 ml), washed with cool water (100 mL), dried over sodium sulphate and evaporated under reduced pressure. The residue was purified by prep-HPLC (Grace C18, 0.1% ammonium bicarbonate in water/acetonitrile) to give the title compound (10 mg, 0.020 mmol, 3.3% yield). ¹H NMR (DMSO-d₆, 400 MHz): b: =7.72 (d, J=1.60 Hz, 1H), 7.71 (d, J=1.60 Hz, 1H), 7.65-7.64 (m, 1H), 7.39-7.37 (m, 2H), 7.43-7.42 (m, 3H), 7.35-7.27 (m, 2H), 7.20-7.19 (m, 1H), 6.43 (s, 1H), 5.58-5.24 (m, 2H), 2.23 (s, 3H). MS (m/z) 478 (M−H)⁻

### Example 277

### 3-(6-Amino-4-methylpyridin-3-yl)-1-(4-fluoro-2-methylphenyl)-6-(trifluoromethyl)-2,3-dihydroquinazolin-4(1H)-one

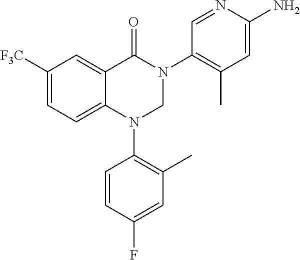 [SMILES: Cc1cc(N)ncc1N1CN(c2ccc(F)cc2C)c2ccc(C(F)(F)F)cc2C1=O]

### Step 1: tert-Butyl (5-(1-(4-fluoro-2-methylphenyl)-4-oxo-6-(trifluoromethyl)-1,4-dihydroquinazolin-3(2H)-yl)-4-methylpyridin-2-yl)carbamate

To a stirred solution of 3-(6-chloro-4-methylpyridin-3-yl)-1-(4-fluoro-2-methylphenyl)-6-(trifluoromethyl)-2,3-dihydroquinazolin-4(1H)-one (350 mg, 0.778 mmol) and tert-butyl carbamate (911 mg, 7.78 mmol) in THF (30 mL) was added cesium carbonate (1268 mg, 3.89 mmol). After this reaction mixture was purged with nitrogen for 10 minutes, XPhos (74.2 mg, 0.156 mmol) and Pd₂(dba)₃ (71.3 mg, 0.078 mmol) were added and the resulting reaction mixture was stirred at 80° C. for 2 hours. The reaction mixture was cooled to room temperature and diluted with water (30 mL). The aqueous layer was extracted with ethyl acetate (2×50 mL). The combined organic phases were dried over anhydrous sodium sulphate (2.8 g) and filtered. The filtrate was concentrated under reduced pressure to give the title compound as a brown solid (410 mg, 0.606 mmol, 78% yield). MS (m/z) 531.2 (M+H)⁺.

### Step 2: 3-(6-Amino-4-methylpyridin-3-yl)-1-(4-fluoro-2-methylphenyl)-6-(trifluoromethyl)-2,3-dihydroquinazolin-4(1H)-one

To a stirred solution of tert-butyl (5-(1-(4-fluoro-2-methylphenyl)-4-oxo-6-(trifluoromethyl)-1,4-dihydroquinazolin-3(2H)-yl)-4-methylpyridin-2-yl)carbamate (410 mg, 0.773 mmol) in Diethyl ether (10 mL) was added hydrochloric acid (1.932 mL, 3.86 mmol) at 0° C. and the resulting reaction mixture was stirred at room temperature for 14 hours. The reaction mixture was concentrated under reduced pressure and the residue was purified by prep-HPLC (X-BRIDGE C18 (19×150 mm) 5 μm column, CH₃CN/H₂O). The solvent was partially concentrated and a white precipitate formed which was filtered and dried under high vacuum to provide the title compound (32 mg, 0.074 mmol, 9.5% yield). ¹H NMR (400 MHz, DMSO-d₆): δ 8.08 (d, J=2.00 Hz, 1H), 7.80 (s, 1H), 7.64 (dd, J=2.00, 8.80 Hz, 1H), 7.45-7.36 (m, 1H), 7.32 (dd, J=2.80, 9.60 Hz, 1H), 7.23-7.15 (m, 1H), 6.40-6.28 (m, 2H), 5.99 (s, 2H), 4.72-5.59 (m, 2H), 2.23 (s, 3H), 2.07 (s, 3H). MS (m/z) 431 (M+H)⁺.

### Example 278

### Mixture of 3-(2-Methyl-6-oxo-1,6-dihydropyridin-3-yl)-1-((1R,2R)-2-methylcyclohexyl)-7-(trifluoromethyl)-2,3-dihydroquinazolin-4(1H)-one and 3-(2-Methyl-6-oxo-1,6-dihydropyridin-3-yl)-1-((1S,2S)-2-methylcyclohexyl)-7-(trifluoromethyl)-2,3-dihydroquinazolin-4(1H)-one

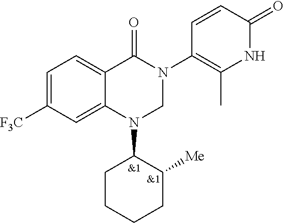 [SMILES: Cc1[nH]c(=O)ccc1N1CN([C@@H]2CCCC[C@H]2C)c2cc(C(F)(F)F)ccc2C1=O]

### Step 1: 3-(2-Methyl-6-oxo-1,6-dihydropyridin-3-yl)-1-(2-methylcyclohexyl)-7-(trifluoromethyl)-2,3-dihydroquinazolin-4(1H)-one

This compound was prepared by methods analogous to those described for Example 26 using 3-(6-methoxy-2-methylpyridin-3-yl)-1-(2-methylcyclohexyl)-7-(trifluoromethyl)-2,3-dihydroquinazolin-4(1H)-one. MS (m/z) 420.4 (M+H)⁺.

### Step 2: Mixture of 3-(2-Methyl-6-oxo-1,6-dihydropyridin-3-yl)-1-((1R,2R)-2-methylcyclohexyl)-7-(trifluoromethyl)-2,3-dihydroquinazolin-4(1H)-one and 3-(2-Methyl-6-oxo-1,6-dihydropyridin-3-yl)-1-((1S,2S)-2-methylcyclohexyl)-7-(trifluoromethyl)-2,3-dihydroquinazolin-4(1H)-one

3-(2-Methyl-6-oxo-1,6-dihydropyridin-3-yl)-1-(2-methylcyclohexyl)-7-(trifluoromethyl)-2,3-dihydroquinazolin-4(1H)-one was applied to chiral HPLC (Agilent Semi-Prep 1200, AD-H (250*20) mm 5μ column, mobile phase: 40:60:0.1 heptane:ethanol:isopropylamine, flow rate 45 ml/min, detecting at 235 nm). The product was further purified by MDAP (XSELECT CSH C18 (150×30) mm 5 μm column, solvent A=0.1% v/v solution of formic acid in water, B=0.1% v/v solution of formic acid in acetonitrile, 30-99% B/A) to give the title compound as a white solid (70 mg, 0.159 mmol, 16.76% yield). MS (m/z) 420.3 (M+H)⁺. ¹H NMR (400 MHz, CHLOROFORM-d) δ: 8.16 (d, J=7. Hz, 1H), 7.35 (d, J=9.3 Hz, 1H), 7.14-7.06 (m, 2H), 6.52 (d, J=9.3 Hz, 1H), 4.88-4.54 (m, 2H), 3.38-3.22 (m, 1H), 2.35 (d, J=6.4 Hz, 3H), 2.00-1.84 (m, 3H), 1.82-1.73 (m, 1H), 1.52-1.13 (m, 5H), 1.03 (d, J=6.4 Hz, 3H). Anal. chiral HPLC RT=3.90 min, 100% on an AD-H column 5μ (150×4.6 mm) eluting with 40:60:0.1 heptane:ethanol:isopropylamine, flow rate 1.0 ml/min, detecting at 254 nm.

Tested in the assay section as a 50/50 mixture of the two compounds

### Examples 279 and 280

### 3-(2-Methyl-6-oxo-1,6-dihydropyridin-3-yl)-1-((1S,2S)-2-methylcyclohexyl)-7-(trifluoromethyl)-2,3-dihydroquinazolin-4(1H)-one and 3-(2-Methyl-6-oxo-1,6-dihydropyridin-3-yl)-1-((1R,2R)-2-methylcyclohexyl)-7-(trifluoromethyl)-2,3-dihydroquinazolin-4(1H)-one

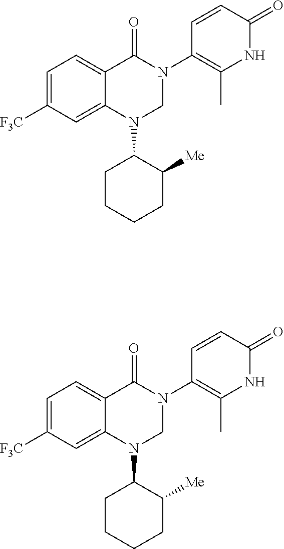 [SMILES: Cc1[nH]c(=O)ccc1N1CN([C@H]2CCCC[C@@H]2C)c2cc(C(F)(F)F)ccc2C1=O ; Cc1[nH]c(=O)ccc1N1CN([C@@H]2CCCC[C@H]2C)c2cc(C(F)(F)F)ccc2C1=O]

Mixture of 3-(2-Methyl-6-oxo-1,6-dihydropyridin-3-yl)-1-((1R,2R)-2-methylcyclohexyl)-7-(trifluoromethyl)-2,3-dihydroquinazolin-4(1H)-one and 3-(2-Methyl-6-oxo-1,6-dihydropyridin-3-yl)-1-((1S,2S)-2-methylcyclohexyl)-7-(trifluoromethyl)-2,3-dihydroquinazolin-4(1H)-one were separated by chiral HPLC (Agilent Semi-Prep 1200, CC4 (250*20) mm 5μ column, mobile phase: 80:20 MeCN, flow rate 1.0 ml/min, detecting at 235 nm) to give

3-(2-methyl-6-oxo-1,6-dihydropyridin-3-yl)-1-((1S,2S)-2-methylcyclohexyl)-7-(trifluoromethyl)-2,3-dihydroquinazolin-4(1H)-one as a white solid (40 mg, 0.094 mmol, 9.98% yield). MS (m/z) 420.3 (M+H)⁺. ¹H NMR (400 MHz, CHLOROFORM-d) δ: 8.14 (d, J=8.07 Hz, 1H), 7.36 (d, J=9.54 Hz, 1H), 7.14-7.07 (m, 2H), 6.54 (d, J=9.54 Hz, 1H), 4.84-4.54 (m, 2H), 3.40-3.25 (m, 1H), 2.35 (d, J=5.87 Hz, 3H), 1.99-1.84 (m, 3H), 1.80-1.59 (m, 2H), 1.50-1.16 (m, 4H), 1.01 (d, J=6.36 Hz, 3H). Anal. chiral HPLC RT=3.79 min, 100% on a CC4 column 5μ (150×4.6) mm eluting with 80:20:0.1 acetonitrile:methanol:isopropylamine, flow rate 1.0 ml/min, detecting at 235 nm.

3-(2-Methyl-6-oxo-1,6-dihydropyridin-3-yl)-1-((1R,2R)-2-methylcyclohexyl)-7-(trifluoromethyl)-2,3-dihydroquinazolin-4(1H)-one (38 mg, 0.090 mmol, 9.48% yield). MS (m/z) 420.3 (M+H)⁺. ¹H NMR (400 MHz, CHLOROFORM-d) δ: ¹H NMR (400 MHz, CHLOROFORM-d) δ: 8.14 (d, J=8.07 Hz, 1H), 7.35 (d, J=9.54 Hz, 1H), 7.15-7.07 (m, 2H), 6.53 (d, J=9.54 Hz, 1H), 4.84-4.56 (m, 2H), 3.39-3.25 (m, 1H), 2.34 (d, J=6.11 Hz, 3H), 1.98-1.85 (m, 3H), 1.82-1.60 (m, 2H), 1.47-1.16 (m, 4H), 1.01 (d, J=6.60 Hz, 3H). Anal. chiral HPLC RT=4.71 min, 98%.

### Examples 281 and 282

### 3-(2-Methyl-6-oxo-1,6-dihydropyridin-3-yl)-1-((3S,4S)-3-methyltetrahydro-2H-pyran-4-yl)-7-(trifluoromethyl)-2,3-dihydroquinazolin-4(1H)-one and 3-(2-Methyl-6-oxo-1,6-dihydropyridin-3-yl)-1-((3R,4R)-3-methyltetrahydro-2H-pyran-4-yl)-7-(trifluoromethyl)-2,3-dihydroquinazolin-4(1H)-one

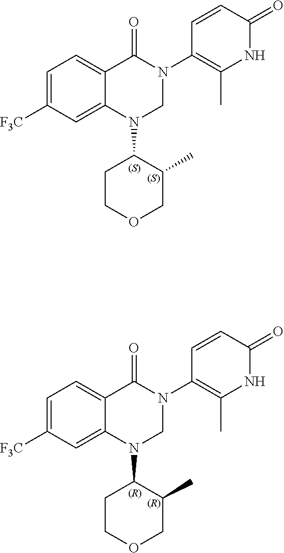

To a solution of cis-rac-3-(6-methoxy-2-methylpyridin-3-yl)-1-((3R,4R)-3-methyltetrahydro-2H-pyran-4-yl)-7-(trifluoromethyl)-2,3-dihydroquinazolin-4(1H)-one (0.283 g, 0.650 mmol) and sodium iodide (0.195 g, 1.300 mmol) in acetonitrile (6.50 ml) was added TMS-Cl (0.166 ml, 1.300 mmol) and heated to 60° C. for 5 hours. The reaction was cooled and dissolved with DCM and water. The layers were separated and the organic layer was washed with water. The combined aqueous layers were extracted with DCM. The combined organics were washed with water, brine and dried with MgSO₄. The residue was purified via Isco Combiflash EZ Prep Reversed phase column C18, load in DMSO, 0-100% ACN (0.1% formic acid)/water (0.1% formic acid) to provide the racemic title compound (0.208 g, 0.494 mmol, 76% yield), MS (m/z) 422 (M+H)⁺. The two enantiomers were separated by chiral SFC on a Chiralcel IG column (250 mm×20 mm) eluting with 70% CO₂ 30% EtOH, flow rate 50 g/min detecting at 220 nm to give:

(3S,4S)-enantiomer (95 mg, 0.225 mmol, 34%). MS (m/z) 422 (M+H)⁺. ¹H NMR (CHLOROFORM-d, 400 MHz) δ 13.05 (br d, 1H, J=2.0 Hz), 8.20 (d, 1H, J=8.3 Hz), 7.36 (d, 1H, J=9.8 Hz), 7.3-7.3 (m, 1H), 7.2-7.2 (m, 1H), 7.11 (s, 1H), 6.51 (t, 1H, J=9.5 Hz), 4.7-5.0 (m, 2H), 4.1-4.2 (m, 1H), 3.98 (tt, 1H, J=4.0, 11.6 Hz), 3.86 (d, 1H, J=11.7 Hz), 3.7-3.8 (m, 1H), 3.56 (br t, 1H, J=11.7 Hz), 2.35 (d, 3H, J=19.1 Hz), 2.2-2.3 (m, 2H), 1.16 (d, 3H, J=6.8 Hz). Anal. SFC RT=4.07 min, 100% on a Chiralcel IG column (150 mm×4.6 mm) eluting with 70% CO₂/30% EtOH, at 30° C., flow rate 3 ml/min, detecting at 220 nm.

(3R, 4R)-enantiomer (95.7 mg, 0.227 mmol, 35%) MS (m/z) 422 (M+H)⁺. ¹H NMR (CHLOROFORM-d, 400 MHz) δ 12.90 (br t, 1H, J=13.0 Hz), 8.20 (d, 1H, J=8.3 Hz), 7.36 (d, 1H, J=9.3 Hz), 7.3-7.3 (m, 1H), 7.2-7.2 (m, 1H), 7.11 (s, 1H), 6.51 (t, 1H, J=9.5 Hz), 4.7-5.0 (m, 2H), 4.15 (br d, 1H, J=11.2 Hz), 3.9-4.0 (m, 1H), 3.86 (d, 1H, J=11.7 Hz), 3.7-3.8 (m, 1H), 3.56 (br t, 1H, J=11.7 Hz), 2.34 (d, 3H, J=19.6 Hz), 2.2-2.3 (m, 2H), 1.16 (d, 3H, J=6.8 Hz) Anal. SFC RT=5.64 min, 100%.

### Examples 283 and 284

### 3-(2-Methyl-6-oxo-1,6-dihydropyridin-3-yl)-1-((3R,4S)-3-methyltetrahydro-2H-pyran-4-yl)-7-(trifluoromethyl)-2,3-dihydroquinazolin-4(1H)-one and 3-(2-Methyl-6-oxo-1,6-dihydropyridin-3-yl)-1-((3S,4R)-3-methyltetrahydro-2H-pyran-4-yl)-7-(trifluoromethyl)-2,3-dihydroquinazolin-4(1H)-one

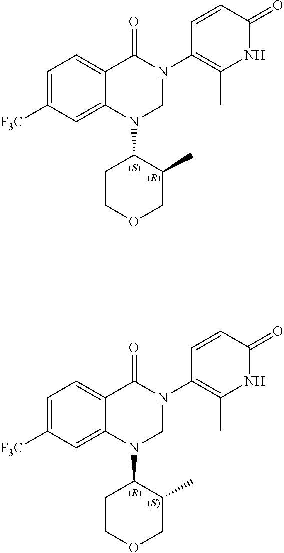

To a solution of trans-rac-3-(6-methoxy-2-methylpyridin-3-yl)-1-((3R,4S)-3-methyltetrahydro-2H-pyran-4-yl)-7-(trifluoromethyl)-2,3-dihydroquinazolin-4(1H)-one (0.063 g, 0.145 mmol) and sodium iodide (0.043 g, 0.289 mmol) in acetonitrile (1.447 ml) was added TMS-Cl (0.037 ml, 0.289 mmol) and heated to 60° C. for 4 hours. The reaction was cooled and dissolved with DCM and water. The layers were separated and the organic layer was washed with water. The combined aqueous layers were extracted with DCM. The combined organics were washed with water, brine and dried with MgSO₄. The residue was purified via column chromatography (Isco, 24 g column, 0-80% (3:1 EtOAc:EtOH)/heptane) to provide the racemic title compound (40 mg, 0.090 mmol, 62% yield). MS (m/z) 422 (M+H)⁺. The two enantiomers were separated by chiral SFC on a Chiral CC4 column (250 mm×20 mm) eluting with 80% CO₂ 20% EtOH, flow rate 50 g/min detecting at 220 nm, 30° C. to give:

(3R,4S)-enantiomer (11.6 mg, 0.028 mmol, 19%). MS (m/z) 422 (M+H)⁺. ¹H NMR (CHLOROFORM-d, 400 MHz) δ: 13.05 (br d, 1H, J=2.0 Hz), 8.20 (d, 1H, J=8.3 Hz), 7.36 (d, 1H, J=9.8 Hz), 7.3-7.3 (m, 1H), 7.2-7.2 (m, 1H), 7.11 (s, 1H), 6.51 (t, 1H, J=9.5 Hz), 4.7-5.0 (m, 2H), 4.1-4.2 (m, 1H), 3.98 (tt, 1H, J=4.0, 11.6 Hz), 3.86 (d, 1H, J=11.7 Hz), 3.7-3.8 (m, 1H), 3.56 (br t, 1H, J=11.7 Hz), 2.35 (d, 3H, J=19.1 Hz), 2.2-2.3 (m, 2H), 1.16 (d, 3H, J=6.8 Hz). Anal. SFC RT=5.37 min, 100% on a Chiral CC4 column (150 mm×4.6 mm) eluting with 75% CO₂/25% EtOH, at 30° C., flow rate 3 ml/min, detecting at 220 nm.

(3S, 4R)-enantiomer (11.6 mg, 0.027 mmol, 19%) MS (m/z) 422 (M+H)⁺. ¹H NMR (CHLOROFORM-d, 400 MHz) δ 13.40 (br s, 1H), 8.18 (d, 1H, J=8.3 Hz), 7.34 (dd, 1H, J=5.9, 9.3 Hz), 7.18 (d, 1H, J=8.3 Hz), 7.14 (d, 1H, J=6.4 Hz), 6.51 (d, 1H, J=9.8 Hz), 4.6-4.9 (m, 2H), 4.1-4.2 (m, 1H), 4.03 (dd, 1H, J=4.4, 11.7 Hz), 3.5-3.6 (m, 2H), 3.20 (t, 1H, J=11.0 Hz), 2.35 (d, 3H, J=7.8 Hz), 1.99 (ddd, 1H, J=4.4, 6.2, 10.4 Hz), 1.8-1.9 (m, 1H), 1.7-1.8 (m, 1H), 0.96 (d, 3H, J=6.4 Hz) Anal. SFC RT=6.72 min, 100%.

### Examples 285 and 286

### rel-(R)-1-(4-Fluoro-2-methylphenyl)-2-methyl-3-(6-oxo-1,6-dihydropyridin-3-yl)-7-(trifluoromethyl)-2,3-dihydroquinazolin-4(1H)-one

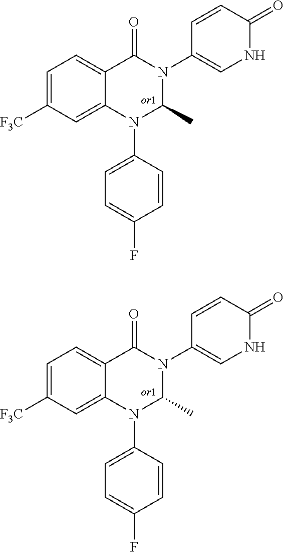 [SMILES: C[C@@H]1N(c2ccc(=O)[nH]c2)C(=O)c2ccc(C(F)(F)F)cc2N1c1ccc(F)cc1 ; C[C@H]1N(c2ccc(=O)[nH]c2)C(=O)c2ccc(C(F)(F)F)cc2N1c1ccc(F)cc1]

The racemic compound 1-(4-fluoro-2-methylphenyl)-2-methyl-3-(6-oxo-1,6-dihydropyridin-3-yl)-7-(trifluoromethyl)-2,3-dihydroquinazolin-4(1H)-one (34 mg, 0.079 mmol) was chiral separated on HPLC with Chiralpak AS-H column using isocratic 95:5 acetonitrile:methanol to give:

Enantiomer-1 (10.0 mg, 0.022 mmol, 28%). MS (m/z) 432.3 (M+H)⁺. ¹H NMR (400 MHz, DMSO-d₆) δ: 11.75 (br. s., 1H) 8.10-8.04 (m, 1H) 7.51-7.12 (m, 6H) 6.60-6.37 (m, 2H) 5.28-5.21 (m, 1H) 2.34 (br. s., 1H) 2.23 (s, 2H) 1.51 (d, J=5.38 Hz, 2H) 1.40 (br. s., 1H). Anal. HPLC RT=5.68 min, 100% on a Chiralpak AS-H column eluting with acetonitrile/methanol 95:5.

Enantiomer-2 (10.0 mg, 0.022 mmol, 28%). MS (m/z) 432.3 (M+H)⁺. ¹H NMR (400 MHz, DMSO-d₆) δ: 11.75 (br. s., 1H) 8.10-8.04 (m, 1H) 7.51-7.12 (m, 6H) 6.60-6.37 (m, 2H) 5.28-5.21 (m, 1H) 2.34 (br. s., 1H) 2.23 (s, 2H) 1.51 (d, J=5.38 Hz, 2H) 1.40 (br. s., 1H). Anal. HPLC RT=9.67 min, 100%.

### Examples 287 and 288

### 1-(4-Fluoro-2-methylphenyl)-3-((2S,3S)-2-methyl-6-oxopiperidin-3-yl)-7-(trifluoromethyl)-2,3-dihydroquinazolin-4(1H)-one and 1-(4-Fluoro-2-methylphenyl)-3-((2R,3R)-2-methyl-6-oxopiperidin-3-yl)-7-(trifluoromethyl)-2,3-dihydroquinazolin-4(1H)-one

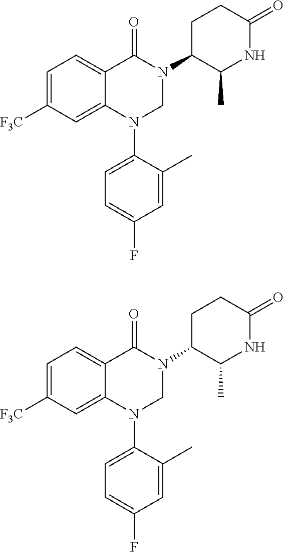 [SMILES: Cc1cc(F)ccc1N1CN([C@H]2CCC(=O)N[C@H]2C)C(=O)c2ccc(C(F)(F)F)cc21 ; Cc1cc(F)ccc1N1CN([C@@H]2CCC(=O)N[C@@H]2C)C(=O)c2ccc(C(F)(F)F)cc21]

The racemic title compound was prepared from 1-(4-fluoro-2-methylphenyl)-3-(2-methyl-6-oxo-1,6-dihydropyridin-3-yl)-7-(trifluoromethyl)-2,3-dihydroquinazolin-4(1H)-one by methods analogous to those described for Example 268. The two enantiomers were separated by chiral HPLC method using Chiralpak IF column (250×4.6) mm 5 micron, eluting with heptane:EtOH:isopropylamine 60:40:0.2, flow rate: 1 ml/min, detecting at 250 nm, 25° C. to give:

1-(4-fluoro-2-methylphenyl)-3-((2S,3S)-2-methyl-6-oxopiperidin-3-yl)-7-(trifluoromethyl)-2,3-dihydroquinazolin-4(1H)-one. (5.5 mg, 0.012 mmol). MS (m/z) 436.4 (M+H)⁺. ¹H NMR (400 MHz, METHANOL-d₄) δ 8.12 (d, J=8.1 Hz, 1H), 7.30 (td, J=8.6, 5.5 Hz, 1H), 7.25-7.18 (m, 2H), 7.12 (td, J=8.3, 2.9 Hz, 1H), 6.43 (br d, J=6.4 Hz, 1H), 5.29-5.02 (m, 1H), 4.95-4.84 (m, 2H), 3.97-3.87 (m, 1H), 2.58-2.38 (m, 2H), 2.28-2.15 (m, 4H), 2.06-1.91 (m, 1H), 1.24 (dd, J=16.3, 6.7 Hz, 3H). Anal. HPLC RT=7.51 min, 100% on a Chiralpak IF column (4.6 mm×25 cm) eluting with 40% heptane/60% EtOH (+0.2% isopropylamine), flow rate 1 ml/min, ambient temperature.

1-(4-fluoro-2-methylphenyl)-3-((2R,3R)-2-methyl-6-oxopiperidin-3-yl)-7-(trifluoromethyl)-2,3-dihydroquinazolin-4(1H)-one. (7 mg, 0.015 mmol). MS (m/z) 436.4 (M+H)⁺. ¹H NMR (400 MHz, METHANOL-d₄) δ: 8.12 (d, J=8.1 Hz, 1H), 7.36-7.26 (m, 1H), 7.25-7.18 (m, 2H), 7.12 (td, J=8.3, 2.9 Hz, 1H), 6.43 (br d, J=6.4 Hz, 1H), 5.24-5.06 (m, 1H), 4.95-4.83 (m, 2H), 3.97-3.86 (m, 1H), 2.59-2.38 (m, 2H), 2.31-2.15 (m, 4H), 2.01 (br s, 1H), 1.24 (dd, J=16.3, 6.7 Hz, 3H). Anal. HPLC RT=9.56 min, 99%.

### Examples 289 and 290

### 3-(2,4-Dimethyl-6-oxo-1,6-dihydropyridin-3-yl)-1-(4-fluoro-2-isopropylphenyl)-7-(trifluoromethyl)-2,3-dihydroquinazolin-4(1H)-one (Atropisomer 1) and 3-(2,4-dimethyl-6-oxo-1,6-dihydropyridin-3-yl)-1-(4-fluoro-2-isopropylphenyl)-7-(trifluoromethyl)-2,3-dihydroquinazolin-4(1H)-one (Atropisomer 2)

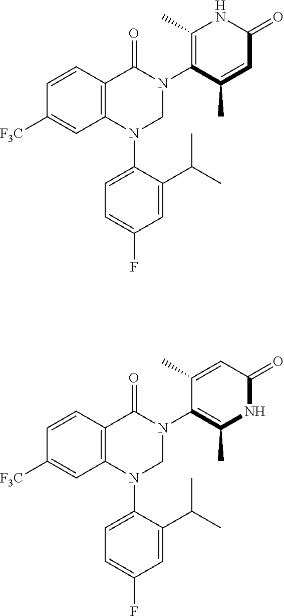

Racemic 3-(2,4-dimethyl-6-oxo-1,6-dihydropyridin-3-yl)-1-(4-fluoro-2-isopropylphenyl)-7(trifluoromethyl)-2,3-dihydroquinazolin-4(1H)-one was separated by chiral SFC on a YMC CELLULOSE-SC (250*30) mm 5μ eluting with 60% CO₂ 40% IPA, flow rate 3 mL/min detecting at 220 nm to give:

Atropisomer 1 (58 mg, 0.121 mmol, 14%). MS (m/z) 474.0 (M+H)⁺. ¹H-NMR (400 MHz, DMSO-d₆) δ: 11.64 (br s, 1H), 8.06 (d, J=8.0 Hz, 1H), 7.48-7.40 (m, 2H), 7.26-7.20 (m, 2H), 6.27 (s, 1H), 6.10 (d, J=4.0 Hz, 1H), 5.52 (dd, J=1.6, 10.0, Hz, 1H), 4.77 (dd, J=10.0, 12.8 Hz, 1H), 3.21-3.10 (m, 1H), 2.14 (d, J=1.6 Hz, 3H), 2.06 (d, J=10.0 Hz, 3H), 1.17 (dd, J=2.8, 6.8 Hz, 3H), 1.10 (d, J=6.8 Hz, 3H). Anal. SFC RT=3.46 min, 99.7% on a YMC CELLULOSE-SC eluting with 60% CO₂ 40% IPA, flow rate 3 mL/min detecting at 220 nm.

Atropisomer 2 (61 mg, 0.128 mmol, 15%). MS (m/z) 474.2 (M+H)⁺. ¹H-NMR (400 MHz, DMSO-d₆) δ: 11.64 (br s, 1H), 8.06 (d, J=8.0 Hz, 1H), 7.48-7.39 (m, 2H), 7.26-7.20 (m, 2H), 6.27 (s, 1H), 6.10 (d, J=4.4 Hz, 1H), 5.52 (dd, J=2.0, 10.0, Hz, 1H), 4.77 (dd, J=9.6, 12.4 Hz, 1H), 3.20-3.10 (m, 1H), 2.14 (d, J=1.6 Hz, 3H), 2.06 (d, J=10.0 Hz, 3H), 1.17 (dd, J=2.4, 6.8 Hz, 3H), 1.10 (d, J=6.8 Hz, 3H). Anal. SFC RT=5.37 min, 99.7% on a YMC CELLULOSE-SC eluting with 60% CO₂ 40% IPA, flow rate 3 mL/min detecting at 220 nm.

### Examples 291 and 292

### 6-Chloro-3-(2,4-dimethyl-6-oxo-1,6-dihydropyridin-3-yl)-1-(4-fluoro-2-isopropylphenyl)-2,3-dihydroquinazolin-4(1H)-one and 6-Chloro-3-(2,4-dimethyl-6-oxo-1,6-dihydropyridin-3-yl)-1-(4-fluoro-2-isopropylphenyl)-2,3-dihydroquinazolin-4(1H)-one

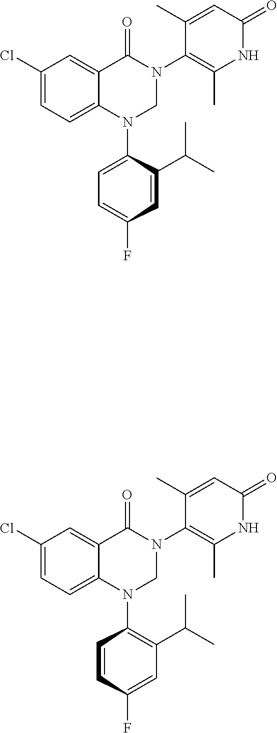

Racemic 6-chloro-3-(2,4-dimethyl-6-oxo-1,6-dihydropyridin-3-yl)-1-(4-fluoro-2-isopropylphenyl)-2,3-dihydroquinazolin-4(1H)-one (125 mg) # was separated by chiral SFC on a CELLULOSE-SC (250*30) mm 5μ eluting with 60% CO₂ 40% MeOH, total flow 70 g/min detecting at 220 nm to give:

Atropisomer 1 (26 mg, 0.059 mmol, 20.8%). MS (m/z) 440.2 (M+H)⁺. ¹H-NMR (400 MHz, DMSO-d₆) δ: 11.63 (br s, 1H), 7.77 (d, J=2.4 Hz, 1H), 7.41-7.36 (m, 3H), 7.19 (td, J=3.2, 8.4 Hz, 1H), 6.16 (d, J=8.8 Hz, 1H), 6.09 (d, J=6.4 Hz, 1H), 5.41 (d, J=9.6 Hz, 1H), 4.68 (t, J=11.2 Hz, 1H), 3.23-3.11 (m, 1H), 2.12 (d, J=8.8 Hz, 3H), 2.04 (d, J=2.0 Hz, 3H), 1.21-1.08 (m, 6H). Anal. SFC RT=3.33 min, >99% on a YMC CELLULOSE-SC eluting with 60% CO₂ 40% MeOH, flow rate 3 mL/min detecting at 220 nm.

Atropisomer 2 (23 mg, 0.052 mmol, 18.4%). MS (m/z) 440.2 (M+H)⁺. ¹H-NMR (400 MHz, DMSO-d₆) δ: 11.63 (brs, 1H), 7.77 (d, J=2.8 Hz, 1H), 7.41-7.36 (m, 3H), 7.18 (td, J=2.8, 8.0 Hz, 1H), 6.16 (d, J=8.8 Hz, 1H), 6.09 (d, J=6.4 Hz, 1H), 5.41 (d, J=10.0 Hz, 1H), 4.68 (dd, J=9.6, 12.0 Hz, 1H), 3.20-3.13 (m, 1H), 2.12 (d, J=9.2 Hz, 3H), 2.04 (d, J=1.6 Hz, 3H), 1.21-1.08 (m, 6H). Anal. SFC RT=4.52 min, >99% on a YMC CELLULOSE-SC eluting with 60% CO₂ 40% MeOH, flow rate 3 mL/min detecting at 220 nm.

### Examples 293 and 294

### 1-(2-(tert-Butyl)-4-fluorophenyl)-6-chloro-3-(2-methyl-6-oxo-1,6-dihydropyridin-3-yl)-2,3-dihydroquinazolin-4(1H)-one and 1-(2-(tert-Butyl)-4-fluorophenyl)-6-chloro-3-(2-methyl-6-oxo-1,6-dihydropyridin-3-yl)-2,3-dihydroquinazolin-4(1H)-one

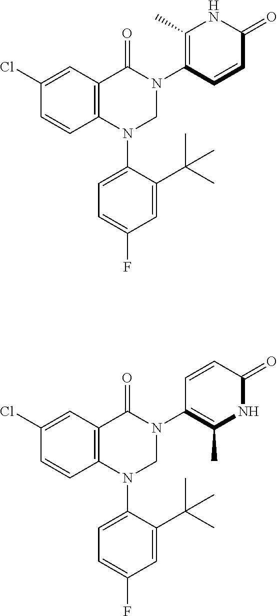

Racemic 1-(2-(tert-butyl)-4-fluorophenyl)-6-chloro-3-(2-methyl-6-oxo-1,6-dihydropyridin-3-yl)-2,3-dihydroquinazolin-4(1H)-one (450 mg) was separated by chiral SFC on a CELLULOSE-SC (250*30) mm 5μ eluting with 60% CO₂ 40% MeOH, total flow 70 g/min, detecting at 220 nm. Each of the atropisomers was purified by reverse phase column chromatography (RediSep Gold C-18 15 g column, Mobile Phase: A: 0.01% HCOOH in H₂O, B: ACN, 0-100% B over 40 min) to give:

Atropisomer 1: (95 mg, 0.214 mmol, 13.87%). MS (m/z) 440.2 (M+H)⁺. ¹HNMR (400 MHz, DMSO-d₆): δ=11.82 (br s, 1H), 7.80-7.76 (m, 1H), 7.45-7.30 (m, 3H), 7.29-7.21 (m, 1H), 7.17 (d, J=9.2 Hz, 1H), 6.20 (dd, J=9.6, 4.0 Hz, 1H), 6.06 (dd, J=8.8, 4.0 Hz, 1H), 5.61 (d, J=9.2 Hz, 0.6H), 5.49 (d, J=9.6 Hz, 0.4H), 4.47 (d, J=9.6 Hz, 0.4H), 4.43 (d, J=8.8 Hz, 0.6H), 2.15-2.05 (m, 3H), 1.38-1.32 (m, 9H). Anal. SFC RT=4.30 min, 97.8% on a Lux A1 column eluting with 60% CO₂ 40% IPA, flow rate 4 mL/min detecting at 220 nm.

Atropisomer 2: (100 mg, 0.226 mmol, 14.67%). MS (m/z) 440.0 (M+H)⁺. ¹HNMR (400 MHz, DMSO-d₆): δ=11.80 (brs, 1H), 7.80-7.76 (m, 1H), 7.45-7.30 (m, 3H), 7.29-7.22 (m, 1H), 7.17 (d, J=9.6 Hz, 1H), 6.20 (dd, J=9.2, 4.0 Hz, 1H), 6.06 (dd, J=8.8, 3.6 Hz, 1H), 5.61 (d, J=9.2 Hz, 0.6H), 5.49 (d, J=9.6 Hz, 0.4H), 4.47 (d, J=9.6 Hz, 0.4H), 4.43 (d, J=9.2 Hz, 0.6H), 2.15-2.05 (m, 3H), 1.38-1.32 (m, 9H). Anal. SFC RT=3.17 min, 98.3%

### Examples 295 and 296

### 1-(4-Fluoro-2-methylphenyl)-3-(1-methyl-6-oxo-1,6-dihydropyridin-3-yl)-7-(trifluoromethyl)-2,3-dihydro quinazolin-4(1H)-one and 1-(4-Fluoro-2-methylphenyl)-3-(1-methyl-6-oxo-1,6-dihydropyridin-3-yl)-7-(trifluoromethyl)-2,3-dihydro quinazolin-4(1H)-one

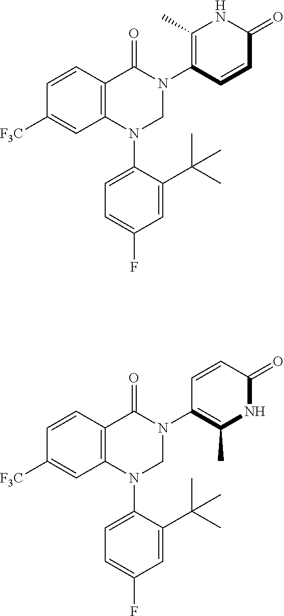

Racemic 1-(2-(tert-butyl)-4-fluorophenyl)-3-(2-methyl-6-oxo-1,6-dihydropyridin-3-yl)-7-(trifluoromethyl)-2,3-dihydroquinazolin-4(1H)-one (75 mg) was separated by chiral SFC (PIC 100, Chiracel OXH (250*30) mm 5μ column, eluting with 60% CO₂ 40% MeOH, total flow 70 g/min detecting at 220 nm) to give:

Atropisomer 1 (30 mg). MS (m/z) 474.0 (M+H)⁺. ¹H NMR (400 MHz, DMSO-d₆): δ=11.82 (br s, 1H), 8.06 (dd, J=8.0, 5.6 Hz, 1H), 7.52-7.15 (m, 5H), 6.21 (dd, J=9.2, 5.6 Hz, 1H), 6.17-6.15 (m, 1H), 5.71 (d, J=9.2 Hz, 0.6H), 5.60 (d, J=9.6 Hz, 0.4H), 4.52 (dd, J=12, 10 Hz, 1H), 2.15-2.05 (m, 3H), 1.38-1.33 (m, 9H). Anal. SFC RT=5.03 min, 99.1% on a Chiralpak OX-H eluting with 60% CO₂ 40% MeOH, flow rate 3 mL/min detecting at 220 nm.

Atropisomer 2 (23 mg). MS (m/z) 474.2 (M+H)⁺. ¹H NMR (400 MHz, DMSO-d₆): δ=11.82 (br s, 1H), 8.06 (dd, J=8.0, 5.6 Hz, 1H), 7.52-7.15 (m, 5H), 6.21 (dd, J=9.6, 5.6 Hz, 1H), 6.17-6.15 (m, 1H), 5.72 (d, J=9.2 Hz, 0.6H), 5.60 (d, J=9.6 Hz, 0.4H), 4.52 (dd, J=12, 9.6 Hz, 1H), 2.15-2.05 (m, 3H), 1.38-1.33 (m, 9H). Anal. SFC RT=3.05 min, 96.9% on a Chiralpak OX-H eluting with 60% CO₂ 40% MeOH, flow rate 3 mL/min detecting at 220 nm.

### Example 297

### 1-(4-fluoro-2-methylphenyl)-3-(2-methyl-6-oxo-1,6-dihydropyridin-3-yl)-6-(trifluoromethyl)-2,3-dihydroquinazolin-4(1H)-one

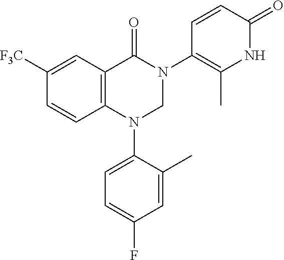 [SMILES: Cc1cc(F)ccc1N1CN(c2ccc(=O)[nH]c2C)C(=O)c2cc(C(F)(F)F)ccc21]

To a solution of 1-(4-fluoro-2-methylphenyl)-3-(6-methoxy-2-methylpyridin-3-yl)-6-(trifluoromethyl)-2,3-dihydroquinazolin-4(1H)-one (0.6 g, 1.4 mmol) in acetonitrile (10 mL) under nitrogen at room temperature was added iodotrimethylsilane (0.54 g, 2.7 mmol) dropwise over 5 min. The reaction mixture was stirred at 80° C. for 12 h. The reaction mixture was cooled to RT and concentrated under vacuum. The crude residue was dissolved in DCM (100 mL) and washed with sat. sodium thiosulphate (20 mL). Organic layer was dried over sodium sulphate and concentrated onto celite. Purification by reverse phase chromatography on C18 (40 g) with 0→100% gradient with 0.1% formic acid in acetonitrile in 0.1% formic acid in water as eluant afforded clean fractions which were concentrated and the resulting precipitate was filtered, washed with water and dried to afford the title compound as a colorless solid (0.21 g, 0.5 mmol, 36% yield). MS (m/z) 432.1 (M+H)⁺. ¹H NMR (400 MHz, DMSO-d₆) δ 11.80 (s, 1H), 8.08 (s, 1H), 7.65 (d, J=8.40 Hz, 1H), 7.45-7.29 (m, 3H), 7.25-7.15 (m, 1H), 6.38-6.15 (m, 2H), 5.51 (d, J=9.20 Hz, 0.6H), 5.07 (d, J=9.20 Hz, 0.4H), 4.94 (d, J=10.40 Hz, 0.4H), 4.78 (d, J=9.20 Hz, 0.6H), 2.23 (s, 3H), 2.12 (s, 3H).

### Example 298

### 1-(2-methyl-4-(trifluoromethoxy)phenyl)-3-(2-methyl-6-oxo-1,6-dihydropyridin-3-yl)-6-(trifluoromethyl)-2,3-dihydropyrido[2,3-d]pyrimidin-4(1H)-one

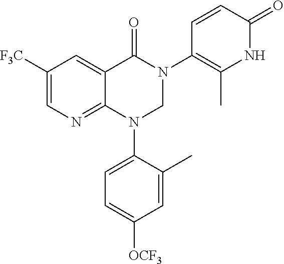 [SMILES: Cc1cc(OC(F)(F)F)ccc1N1CN(c2ccc(=O)[nH]c2C)C(=O)c2cc(C(F)(F)F)cnc21]

To a solution containing 3-(6-methoxy-2-methylpyridin-3-yl)-1-(2-methyl-4-(trifluoromethoxy)phenyl)-6-(trifluoromethyl)-2,3-dihydropyrido[2,3-d]pyrimidin-4(1H)-one (90.24 g, 176 mmol) and sodium iodide (174 g, 1162 mmol) in acetonitrile (766 mL) was added trimethylchlorosilane (149 mL, 1162 mmol). The reaction was stirred at 80° C. for 3 hr. Reaction was cooled, quenched with water and stirred while adding solid sodium metabisulfite—until color change not observed after addition. Extracted with ethyl acetate, washed with sat. sodium metabisulfite solution, washed with brine and dried over MgSO₄, filtered and concentrated. Purification by flash chromatography on SiO₂ (1.5 kg) with 0→30% 25% ethanol in ethyl acetate in ethyl acetate in step gradient with 10% step increase and 3 column volumes per step as eluant afforded product. Impure fractions were combined and purified by flash chromatography on SiO₂ (330 g) with 0→4% methanol in dichloromethane as eluant using step gradient 2% each step 4 column volumes afforded product. The batches of product were combined dried in vacuum oven for 18 hr at 50° C. to afford title compound as a pale yellow foam. (63.41 g, 127 mmol, 72% yield). MS (m/z) 499.2 (M+H)⁺. ¹H NMR (400 MHz, DMSO-d₆) δ 11.82 (br s, 1H), 8.60 (d, J=2.0 Hz, 1H), 8.32 (d, J=2.4 Hz, 1H), 7.47 (br d, J=7.3 Hz, 1H), 7.44-7.38 (m, 2H), 7.32 (br d, J=8.3 Hz, 1H), 6.22 (br d, J=8.8 Hz, 1H), 5.63 (br d, J=9.8 Hz, 0.6H), 5.38 (br d, J=9.8 Hz, 0.4H), 5.23-5.15 (m, 0.4H), 4.96 (br d, J=9.8 Hz, 0.6H), 2.24 (s, 3H), 2.13 (br s, 3H).

### Example 299

### 1-(4-fluoro-2-methylphenyl)-3-(2-methyl-6-oxo-1,6-dihydropyridin-3-yl)-6-(trifluoromethyl)-2,3-dihydropyrido[2,3-d]pyrimidin-4(1H)-one

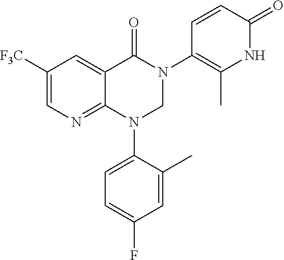 [SMILES: Cc1cc(F)ccc1N1CN(c2ccc(=O)[nH]c2C)C(=O)c2cc(C(F)(F)F)cnc21]

To a solution containing 1-(4-fluoro-2-methylphenyl)-3-(6-methoxy-2-methylpyridin-3-yl)-6-(trifluoromethyl)-2,3-dihydropyrido[2,3-d]pyrimidin-4(1H)-one (370 mg, 0.83 mmol) and trimethylchlorosilane (699 μl, 5.5 mmol) in acetonitrile (3.6 mL) was added sodium iodide (820 mg, 5.5 mmol). The reaction was stirred at 80° C. for 1 hr. Reaction was cooled, quenched with water and brine. Extracted with ethyl acetate, dried over MgSO₄, filtered and concentrated. Purification by flash chromatography on SiO₂ (120 g) with 0→10% methanol in dichloromethane as eluant afforded title compound as a colorless solid (251 mg, 0.58 mmol, 70% yield). ¹H NMR (400 MHz, DMSO-d₆) δ 11.82 (br s, 1H), 8.58 (d, J=1.5 Hz, 1H), 8.30 (d, J=2.4 Hz, 1H), 7.47-7.33 (m, 2H), 7.25 (dd, J=9.5, 2.2 Hz, 1H), 7.14 (td, J=8.3, 2.9 Hz, 1H), 6.26-6.17 (m, 1H), 5.60 (br d, J=9.8 Hz, 0.6H), 5.32 (br d, J=9.8 Hz, 0.4H), 5.17 (br d, J=10.3 Hz, 0.4H), 4.92 (d, J=9.8 Hz, 0.6H), 2.21 (s, 3H), 2.18-2.11 (m, 3H). MS (m/z) 433.2 (M+H)⁺.

### Formulation Example 1—Capsule Composition

An oral dosage form for administering the present invention is produced by filing a standard two-piece hard gelatin capsule with the ingredients shown in Formulation Table 1′, below.

Formulation Table 1′

| | |
|---|---|
| INGREDIENTS | AMOUNTS |

1-(4-fluoro-2-methylphenyl)-3-(2-methyl-6-oxo-1,6-
dihydropyridin-3-yl)-7-(trifluoromethyl)-2,3-
dihydroquinazolin-4(1H)-one (Compound of Example 1)
Lactose
Talc
Magnesium Stearate

### Formulation Example 2—Injectable Parenteral Composition

An injectable form for administering the present invention is produced by stirring 1.7% by weight of 1-(4-fluoro-2-methylphenyl)-3-(6-oxo-1,6-dihydropyridin-3-yl)-7-(trifluoromethyl)-2,3-dihydroquinazolin-4(1H)-one (Compound of Example 2) in 10% by volume propylene glycol in water.

### Formulation Example 3 Tablet Composition

The sucrose, calcium sulfate dihydrate and a Nav1.8 inhibitor as shown in Formulation Table 2′ below, are mixed and granulated with a 10% gelatin solution. The wet granules are screened, dried, mixed with the starch, talc and stearic acid, screened and compressed into a tablet.

Formulation Table 2′

| | |
|---|---|
| INGREDIENTS | AMOUNTS |

1-cyclohexyl-3-(6-oxo-1,6-dihydropyridin-3-yl)-7-
(trifluoromethyl)-2,3-dihydroquinazolin-4(1H)-one
(Compound of Example 3)
calcium sulfate dihydrate
Sucrose
Starch
Talc
stearic acid

It is to be understood that the invention is not limited to the embodiments illustrated hereinabove and the right is reserved to the illustrated embodiments and all modifications coming within the scope of the following claims.

1. A compound represented by the following Formula (X): 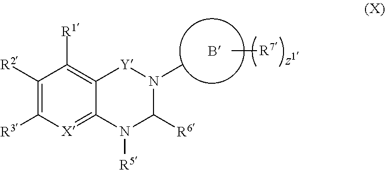
where:
Y′ is selected from: CH₂, C═O and C═S;
X′ is N or C—R^{4′};
wherein:
R^{4′} is selected from: hydrogen, halogen, —C≡N, —NR^{a}R^{b}, straight or branched-(C₁₋₆)-alkyl, —OR′ and —S(O)ₚR^{d},
wherein, when R^{4′} is straight or branched —(C₁₋₆)-alkyl or —OR^{c}, the alkyl chain, when present, is optionally substituted with one to six substituents independently selected from: halogen, —C≡N, oxo, —NR^{a}R^{b}, straight or branched-(C₁₋₆)-alkyl, straight or branched-(C₁₋₆)-haloalkyl and —OR^{c};
R^{1′}, R^{2′} and R^{3′} are independently selected from: hydrogen, halogen, —C≡N, —NR^{a}R^{b}, straight or branched-(C₁₋₆)-alkyl, carbocyclic, heterocyclic, bicycloalkyl, —OR′ and —S(O)ₚR^{d},
wherein, when any of R¹, R^{2′} and R^{3′} is a straight or branched-(C₁₋₆)-alkyl, or —OR^{c}, the alkyl chain, when present, is optionally substituted with one to six substituents independently selected from: halogen, —C≡N, —NR^{a}R^{b}, straight or branched-(C₁₋₆)-alkyl, straight or branched-(C₁₋₆)-haloalkyl, oxo and —OR^{c};
R^{5′} is selected from: carbocyclic, —CH₂-unsaturated carbocyclic, heterocyclic, or bicycloalkyl,
wherein, R^{5′} is optionally substituted with from one to four substituents independently selected from: —C≡N, —NR^{a}R^{b}, halogen, oxo, —C(O)NHR^{a}, —C(O)NR^{a}R^{b}, straight or branched-(C₁₋₆)-alkyl, —OR^{c}, and (C₃₋₆)cycloalkyl,
wherein each of: straight or branched-(C₁₋₆)-alkyl, the alkyl chain of —OR^{c}, when present, and (C₃₋₆)-cycloalkyl is optionally substituted with one to six substituents independently selected from: halogen, oxo, —OH, —NH₂, —NHC₁₋₄alkyl, —N(C₁₋₄alkyl)₂, —OC₁₋₄alkyl and —OC₁₋₄alkyl substituted with from 1 to 6 substituents independently selected from: fluoro, oxo, —OH, —NH₂, —NHC₁₋₄alkyl, —N(C₁₋₄alkyl)₂, —OC₁₋₃alkyl, and —OC₁₋₃alkyl substituted 1 to 6 times by fluoro;
R^{6′} is hydrogen, oxo, straight or branched-(C₁₋₆)-alkyl or straight or branched-(C₁₋₆)-haloalkyl;
B′ is selected from: aryl, heterocycloalkyl, and heteroaryl;
each R^{7′} is independently selected from: halogen, oxo, —C≡N, —NR^{a}R^{b}, —OR^{c}, —S(O)ₚR^{d}, straight or branched (C₁₋₆) alkyl, bicycloalkyl and (C₃₋₆)-cycloalkyl,
wherein, when R^{7′} is a straight or branched —(C₁₋₆)-alkyl, or —OR^{c}, the alkyl chain, when present, is optionally substituted with one to three substituents independently selected from: halogen, —C≡N, —NR^{a}R^{b}, straight or branched-(C₁₋₆)-alkyl, straight or branched-(C₁₋₆)-haloalkyl, oxo and —OR^{c};
R^{d} is hydrogen, —OH, —NR^{a}R^{b}, straight or branched-(C₁₋₆)-alkyl, straight or branched-(C₁₋₆)-haloalkyl or (C₃₋₆)-cycloalkyl;
in each occurrence, R^{a}, R^{b} and R^{c} are independently selected from: hydrogen, straight or branched-(C₁₋₆)-alkyl, and (C₃₋₆)-cycloalkyl,
wherein each of: straight or branched-(C₁₋₆)-alkyl, and (C₃₋₆)-cycloalkyl is optionally substituted with one to six substituents independently selected from: halogen, oxo, —OH, —NH₂, —NHC₁₋₄alkyl, —N(C₁₋₄alkyl)₂, —OC₁₋₄alkyl and —OC₁₋₄alkyl substituted with from 1 to 6 substituents independently selected from: fluoro, oxo, —OH, —NH₂, —NHC₁₋₄alkyl, —N(C₁₋₄alkyl)₂, —OC₁₋₃alkyl, and —OC₁₋₃alkyl substituted 1 to 6 times by fluoro;
z^{1′} is an integer from 0 to 5; and
p is 0, 1 or 2;
or a tautomer thereof,
or a pharmaceutically acceptable salt thereof.

2. The compound according to claim 1 wherein B′ is selected from: pyridinyl, pyrimidinyl, phenyl, pyridazinyl, tetrahydrothiophenyl, pyrazolyl, piperidinyl, tetrahydrothiopyranyl, dihydropyrimidinyl, tetrahydropyridinyl, pyrazinyl, furanyl and hexahydropyrimidinyl.

3. The compound according to claim 1 wherein R^{5′} is phenyl, where phenyl is optionally substituted with from one to four substituents independently selected from: —C≡N, —NR^{a}R^{b}, halogen, oxo, —C(O)NHR^{a}, —C(O)NR^{a}R^{b}, straight or branched-(C₁₋₆)-alkyl, —OR^{c}, and (C₃₋₆)cycloalkyl,
wherein each of: straight or branched-(C₁₋₆)-alkyl, the alkyl chain of —OR^{c}, when present, and (C₃₋₆)-cycloalkyl is optionally substituted with one to six substituents independently selected from: halogen, oxo, —OH, —NH₂, —NHC₁₋₄alkyl, —N(C₁₋₄alkyl)₂, —OC₁₋₄alkyl and —OC₁₋₄alkyl substituted with from 1 to 6 substituents independently selected from: fluoro, oxo, —OH, —NH₂, —NHC₁₋₄alkyl, —N(C₁₋₄alkyl)₂, —OC₁₋₃alkyl, and —OC₁₋₃alkyl substituted 1 to 6 times by fluoro.

4. The compound according to claim 1 wherein X′ is N.

5. The compound according to claim 1 wherein X′ is C—H.

6. The compound according to claim 1 wherein R^{1′}, R^{2′} and R^{3′} are independently selected from: hydrogen, fluoro, chloro, bromo, —C≡N, —CH₃, —CF₃, —CHF₂, —OCH₃, —OCH₂CF₃, —OCHF₂, —OCF₃, and —CF₂CH₂OH.

7. (canceled)

8. The compound according to claim 1 represented by the following Formula (XIV): 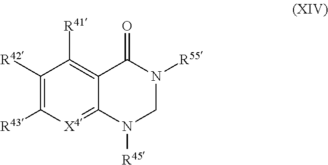
where:
X^{4′} is N or C—R^{4′};
wherein:
R^{44′} is selected from: hydrogen, fluoro, chloro, bromo, and —CH₃;
R^{41′}, R^{42′} and R^{43′} are independently selected from: hydrogen, fluoro, chloro, bromo, —CH₃, —CF₃, —CHF₂, —OCH₃, —OCH₂CF₃, —OCHF₂, —OCF₃, —CF₂CH₂OH, —N(CH₃)₂, —NHCH₃, —C≡N, —OH, —S(O)₂CH₃, cyclopropyl, and oxetanyl;
R^{45′} is selected from: phenyl, cyclopentyl, cyclohexyl, 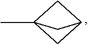
thiophenyl, thiazolyl, pyridyl, tetrahydropyranyl, and —CH₂-phenyl,
wherein, R^{45′} is optionally substituted with from one to four substituents independently selected from: fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(CH₃)₂, —NHCH₃, —C≡N, —OH, and cyclopropyl; and
R^{55′} is selected from: 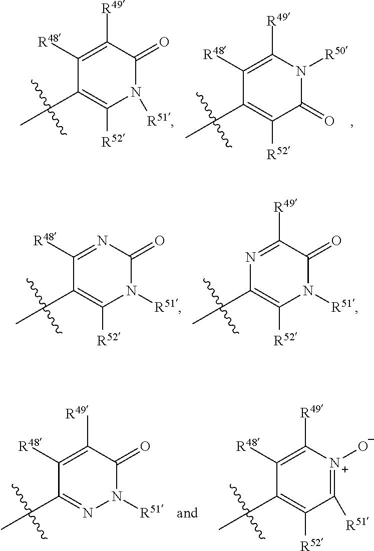
or a corresponding tautomer form thereof,
wherein, R^{48′}, R^{49′}, R^{50′}, R^{51′} and R^{52′} are independently selected from: hydrogen, fluoro, chloro, bromo, —CH₃, —CH₂CH₃, —CH₂CF₃, —CH(CH₃)₂, —C(CH₃)₃, —CF₃, —CF₂CH₂OH, —C(O)NH₂, —OCH₃, —NH₂, —OCH₂CH₃, —OCHF₂, —OCF₃, —OCH₂CH₂OH, —N(CH₃)₂, —NHCH₃, —C≡N, —OH, —C(O)OH, —C(O)CH₃, —OCH₂C(O)OH, —NC(O)CH₃, —NHCH₂CH₂OH, —S(O)₂CH₃, —S(O)₂NH₂, and cyclopropyl;
or a tautomer thereof,
or a pharmaceutically acceptable salt thereof.

9. The compound according to claim 8 wherein X^{4′} is N.

10. The compound according to claim 8 wherein X^{4′} is C—H.

11. The compound according to claim 1 which is selected from:
1-(4-fluoro-2-methylphenyl)-3-(1-methyl-6-oxo-1,6-dihydropyridin-3-yl)-7-(trifluoromethyl)-2,3-dihydro quinazolin-4(1H)-one;
1-(4-fluoro-2-methylphenyl)-3-(pyridin-3-yl)-7-(trifluoromethyl)-2,3-dihydroquinazolin-4(1H)-one;
1-(4-fluoro-2-methylphenyl)-3-(2-methoxypyrimidin-5-yl)-7-(trifluoromethyl)-2,3-dihydroquinazolin-4(1H)-one;
1-(4-fluoro-2-methylphenyl)-3-(1-methyl-1H-pyrazol-5-yl)-6-(trifluoromethyl)-2,3-dihydroquinazolin-4(1H)-one;
N-(3-(1-(4-fluoro-2-methylphenyl)-4-oxo-7-(trifluoromethyl)-1,4-dihydroquinazolin-3(2H)-yl)phenyl)acetamide;
N-(4-(1-(4-fluoro-2-methylphenyl)-4-oxo-7-(trifluoromethyl)-1,4-dihydroquinazolin-3(2H)-yl)phenyl)acetamide;
5-(6-chloro-5-fluoro-1-(4-fluoro-2-methylphenyl)-4-oxo-1,4-dihydroquinazolin-3(2H)-yl)picolinamide;
4-(6-chloro-5-fluoro-1-(4-fluoro-2-methylphenyl)-4-oxo-1,4-dihydroquinazolin-3(2H)-yl)picolinamide;
1-(2-bromo-4-fluorophenyl)-3-(6-methoxy-2-methylpyridin-3-yl)-6-(trifluoromethyl)-2,3-dihydroquinazolin-4(1H)-one;
1-(4-fluoro-2-methylphenyl)-3-(6-methoxypyridin-3-yl)-7-(trifluoromethyl)-2,3-dihydroquinazolin-4(1H)-one;
6-chloro-7-fluoro-1-(2-methyl-4-(trifluoromethoxy)phenyl)-3-(3-methylpyridin-4-yl)-2,3-dihydroquinazolin-4(1H)-one;
6-chloro-1-(2-ethyl-4-fluorophenyl)-7-fluoro-3-(3-methylpyridin-4-yl)-2,3-dihydroquinazolin-4(1H)-one;
1-(4-fluoro-2-methylphenyl)-3-(4-(methylsulfonyl)phenyl)-7-(trifluoromethyl)-2,3-dihydroquinazolin-4(1H)-one;
1-(4-fluoro-2-methylphenyl)-3-(3-(methylsulfonyl)phenyl)-7-(trifluoromethyl)-2,3-dihydroquinazolin-4(1H)-one;
3-(1,1-dioxidotetrahydrothiophen-3-yl)-1-(4-fluoro-2-methylphenyl)-6-(trifluoromethyl)-2,3-dihydroquinazolin-4(1H)-one;
1-(4-fluoro-2-methylphenyl)-3-(3-methylpyridin-4-yl)-6-(trifluoromethyl)-2,3-dihydroquinazolin-4(1H)-one;
3-(1,1-dioxidotetrahydro-2H-thiopyran-4-yl)-1-(4-fluoro-2-methylphenyl)-7-(trifluoromethyl)-2,3-dihydroquinazolin-4(1H)-one;
1-(4-fluoro-2-methylphenyl)-3-(1H-pyrazol-4-yl)-6-(trifluoromethyl)-2,3-dihydroquinazolin-4(1H)-one;
1-(4-fluoro-2-methylphenyl)-3-(6-methoxy-2-methylpyridin-3-yl)-6-(trifluoromethyl)-2,3-dihydroquinazolin-4(1H)-one;
1-(4-fluoro-2-methylphenyl)-3-(4-methyl-6-oxo-1,6-dihydropyridazin-3-yl)-7-(trifluoromethyl)-2,3-dihydroquinazolin-4(1H)-one;
1-(2-methyl-4-(trifluoromethoxy)phenyl)-3-(3-methylpyridin-4-yl)-6-(trifluoromethyl)-2,3-dihydroquinazolin-4(1H)-one;
3-(6-chloro-4-methylpyridin-3-yl)-1-(4-fluoro-2-methylphenyl)-6-(trifluoromethyl)-2,3-dihydroquinazolin-4(1H)-one;
6-chloro-7-(difluoromethyl)-1-(4-fluoro-2-methylphenyl)-3-(3-methylpyridin-4-yl)-2,3-dihydroquinazolin-4(1H)-one;
1-(4-fluoro-2-methylphenyl)-3-(pyridin-3-yl)-6-(trifluoromethyl)-2,3-dihydroquinazolin-4(1H)-one;
1-(4-fluoro-2-methylphenyl)-3-(4-methylpyridin-3-yl)-6-(trifluoromethyl)-2,3-dihydroquinazolin-4(1H)-one;
1-(4-fluoro-2-methylphenyl)-3-(3-methylpyridazin-4-yl)-6-(trifluoromethyl)-2,3-dihydroquinazolin-4(1H)-one;
7-chloro-6-fluoro-1-(4-fluoro-2-methylphenyl)-3-(6-methoxy-2-methylpyridin-3-yl)-2,3-dihydroquinazolin-4(1H)-one;
1-(4-fluoro-2-methylphenyl)-3-(3-methyl-1H-pyrazol-4-yl)-6-(trifluoromethyl)-2,3-dihydroquinazolin-4(1H)-one;
3-(1-acetylpiperidin-4-yl)-1-(4-fluoro-2-methylphenyl)-6-(trifluoromethyl)-2,3-dihydroquinazolin-4(1H)-one;
1-(4-fluoro-2-methylphenyl)-3-(pyridazin-4-yl)-6-(trifluoromethyl)-2,3-dihydroquinazolin-4(1H)-one;
1-(4-fluoro-2-methylphenyl)-3-(5-methylpyridin-3-yl)-6-(trifluoromethyl)-2,3-dihydroquinazolin-4(1H)-one;
1-(4-fluoro-2-methylphenyl)-3-(2-methylpyridin-3-yl)-6-(trifluoromethyl)-2,3-dihydroquinazolin-4(1H)-one;
1-(4-fluoro-2-methylphenyl)-3-((2R,3S)-2-methyl-6-oxopiperidin-3-yl)-6-(trifluoromethyl)-2,3-dihydroquinazolin-4(1H)-one;
3-(1-(4-fluoro-2-methylphenyl)-4-oxo-6-(trifluoromethyl)-1,4-dihydroquinazolin-3(2H)-yl)piperidine-2,6-dione;
1-(4-Fluoro-2-methylphenyl)-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-3-(6-oxo-1,6-dihydropyridin-3-yl)-7-(trifluoromethyl)-2,3-dihydroquinazolin-4(1H)-one;
1-cyclohexyl-3-(6-oxo-1,6-dihydropyridin-3-yl)-7-(trifluoromethyl)-2,3-dihydroquinazolin-4(1H)-one;
1-(4-fluoro-2,6-dimethylphenyl)-3-(6-oxo-1,6-dihydropyridin-3-yl)-7-(trifluoromethyl)-2,3-dihydroquinazolin-4(1H)-one;
1-(4-fluoro-2-methylphenyl)-2-methyl-3-(6-oxo-1,6-dihydropyridin-3-yl)-7-(trifluoromethyl)-2,3-dihydroquinazolin-4(1H)-one;
1-(2-chloro-4-fluorophenyl)-3-(6-oxo-1,6-dihydropyridin-3-yl)-7-(trifluoromethyl)-2,3-dihydroquinazolin-4(1H)-one;
1-(4-fluoro-2-(2-hydroxyethoxy)phenyl)-3-(6-oxo-1,6-dihydropyridin-3-yl)-7-(trifluoromethyl)-2,3-dihydroquinazolin-4(1H)-one;
1-(2,4-difluorophenyl)-3-(6-oxo-1,6-dihydropyridin-3-yl)-7-(trifluoromethyl)-2,3-dihydroquinazolin-4(1H)-one;
1-(2-ethyl-4-fluorophenyl)-3-(6-oxo-1,6-dihydropyridin-3-yl)-7-(trifluoromethyl)-2,3-dihydroquinazolin-4(1H)-one;
8-chloro-1-(4-fluoro-2-methylphenyl)-3-(6-oxo-1,6-dihydropyridin-3-yl)-7-(trifluoromethyl)-2,3-dihydroquinazolin-4(1H)-one;
1-(4-fluoro-2-methylphenyl)-8-methyl-3-(6-oxo-1,6-dihydropyridin-3-yl)-7-(trifluoromethyl)-2,3-dihydroquinazolin-4(1H)-one;
3-(2-methyl-6-oxo-1,6-dihydropyridin-3-yl)-1-(2-methylpyridin-3-yl)-7-(trifluoromethyl)-2,3-dihydroquinazolin-4(1H)-one;
1-(4-fluoro-2-isopropylphenyl)-3-(2-methyl-6-oxo-1,6-dihydropyridin-3-yl)-7-(trifluoromethyl)-2,3-dihydroquinazolin-4(1H)-one;
6-chloro-1-(4-fluoro-2-methylphenyl)-3-(2-methyl-6-oxo-1,6-dihydropyridin-3-yl)-2,3-dihydroquinazolin-4(1H)-one;
1-(4-fluorophenyl)-3-(2-methyl-6-oxo-1,6-dihydropyridin-3-yl)-2,3-dihydroquinazolin-4(1H)-one;
3-(2-methyl-6-oxo-1,6-dihydropyridin-3-yl)-1-(o-tolyl)-2,3-dihydroquinazolin-4(1H)-one;
1-(4-fluoro-2-methylphenyl)-6-methyl-3-(2-methyl-6-oxo-1,6-dihydropyridin-3-yl)-2,3-dihydroquinazolin-4(1H)-one;
1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-3-(6-methyl-2-oxo-1,2-dihydropyridin-4-yl)-7-(trifluoromethyl)-2,3-dihydroquinazolin-4(1H)-one;
1-(4-fluoro-2-methylphenyl)-3-(5-methyl-2-oxo-1,2-dihydropyridin-4-yl)-7-(trifluoromethyl)-2,3-dihydroquinazolin-4(1H)-one;
1-(4-fluoro-2-methylphenyl)-3-(3-methyl-2-oxo-1,2-dihydropyridin-4-yl)-7-(trifluoromethyl)-2,3-dihydroquinazolin-4(1H)-one;
1-(4-fluoro-2-methylphenyl)-3-(2-methyl-6-oxo-1,6-dihydropyridin-3-yl)-7-(trifluoromethoxy)-2,3-dihydroquinazolin-4(1H)-one;
1-(4-fluoro-2-methylphenyl)-7-methyl-3-(2-methyl-6-oxo-1,6-dihydropyridin-3-yl)-2,3-dihydroquinazolin-4(1H)-one;
1-(4-fluoro-2-methylphenyl)-3-(3-methyl-5-oxo-4,5-dihydropyrazin-2-yl)-7-(trifluoromethyl)-2,3-dihydroquinazolin-4(1H)-one;
6-(1-(4-fluoro-2-methylphenyl)-4-oxo-7-(trifluoromethyl)-1,4-dihydroquinazolin-3(2H)-yl)pyrimidine-2,4(1H,3H)-dione;
1-(2-ethyl-4-fluorophenyl)-3-(2-methyl-6-oxo-1,6-dihydropyridin-3-yl)-6-(trifluoromethyl)-2,3-dihydroquinazolin-4(1H)-one;
6-chloro-1-(4-fluoro-2-methylphenyl)-3-(4-methyl-2-oxo-1,2-dihydropyrimidin-5-yl)-2,3-dihydroquinazolin-4(1H)-one;
3-(2,4-dimethyl-6-oxo-1,6-dihydropyridin-3-yl)-1-(4-fluoro-2-isopropylphenyl)-7-(trifluoromethyl)-2,3-dihydroquinazolin-4(1H)-one;
1-(4-fluoro-2-isopropylphenyl)-3-(6-methoxy-2,4-dimethylpyridin-3-yl)-7-(trifluoromethyl)-2,3-dihydroquinazolin-4(1H)-one;
3-(2-ethyl-6-oxo-1,6-dihydropyridin-3-yl)-1-(4-fluoro-2-methylphenyl)-6-(trifluoromethyl)-2,3-dihydroquinazolin-4(1H)-one;
7-(dimethylamino)-1-(4-fluoro-2-methylphenyl)-3-(2-methyl-6-oxo-1,6-dihydropyridin-3-yl)-2,3-dihydroquinazolin-4(1H)-one;
7-chloro-1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-3-(2-methyl-6-oxo-1,6-dihydropyridin-3-yl)-7-(methylamino)-2,3-dihydroquinazolin-4(1H)-one;
6-chloro-1-(2-ethoxy-4-fluorophenyl)-3-(2-methyl-6-oxo-1,6-dihydropyridin-3-yl)-2,3-dihydroquinazolin-4(1H)-one;
1-(4-fluoro-2-isopropylphenyl)-7-methyl-3-(2-methyl-6-oxo-1,6-dihydropyridin-3-yl)-2,3-dihydroquinazolin-4(1H)-one;
7-(difluoromethyl)-6-fluoro-1-(4-fluoro-2-methylphenyl)-3-(2-methyl-6-oxo-1,6-dihydropyridin-3-yl)-2,3-dihydroquinazolin-4(1H)-one;
6-fluoro-1-(4-fluoro-2-methylphenyl)-7-methyl-3-(2-methyl-6-oxo-1,6-dihydropyridin-3-yl)-2,3-dihydropyrido[2,3-d]pyrimidin-4(1H)-one;
6-chloro-1-(4-fluoro-2-methylphenyl)-7-methyl-3-(2-methyl-6-oxo-1,6-dihydropyridin-3-yl)-2,3-dihydropyrido[2,3-d]pyrimidin-4(1H)-one;
6,7-dichloro-1-(4-fluoro-2-methylphenyl)-3-(2-methyl-6-oxo-1,6-dihydropyridin-3-yl)-2,3-dihydropyrido[2,3-d]pyrimidin-4(1H)-one;
6-chloro-5-fluoro-1-(2-methyl-4-(trifluoromethoxy)phenyl)-3-(2-methyl-6-oxo-1,6-dihydropyridin-3-yl)-2,3-dihydroquinazolin-4(1H)-one;
1-(4-fluoro-2-methylphenyl)-3-(2-methyl-6-oxo-1,6-dihydropyridin-3-yl)-4-oxo-1,2,3,4-tetrahydroquinazoline-6-carbonitrile;
6-chloro-1-(4-fluoro-2-isopropylphenyl)-3-(2-methyl-6-oxo-1,6-dihydropyridin-3-yl)-2,3-dihydroquinazolin-4(1H)-one;
1-(4-fluoro-3-hydroxy-2-methylphenyl)-3-(2-methyl-6-oxo-1,6-dihydropyridin-3-yl)-7-(trifluoromethyl)-2,3-dihydroquinazolin-4(1H)-one;
6-chloro-5-fluoro-1-(4-fluoro-2-methylphenyl)-3-(6-oxo-1,6-dihydropyridin-3-yl)-2,3-dihydroquinazolin-4(1H)-one;
6-chloro-5-fluoro-1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-3-(2-methyl-6-oxo-1,6-dihydropyridin-3-yl)-7-(methylsulfonyl)-2,3-dihydroquinazolin-4(1H)-one;
6-chloro-1-(2-ethyl-4-fluorophenyl)-3-(2-methyl-6-oxo-1,6-dihydropyridin-3-yl)-2,3-dihydroquinazolin-4(1H)-one;
6-chloro-3-(2,4-dimethyl-6-oxo-1,6-dihydropyridin-3-yl)-1-(4-fluoro-2-isopropylphenyl)-2,3-dihydroquinazolin-4(1H)-one;
3-(2-ethyl-6-oxo-1,6-dihydropyridin-3-yl)-1-(4-fluoro-2-isopropylphenyl)-7-(trifluoromethyl)-2,3-dihydroquinazolin-4(1H)-one;
1-(4-fluoro-2-methylphenyl)-3-(5-oxo-4,5-dihydropyrazin-2-yl)-7-(trifluoromethyl)-2,3-dihydroquinazolin-4(1H)-one;
6-fluoro-1-(4-fluoro-2-methylphenyl)-7-methyl-3-(2-methyl-6-oxo-1,6-dihydropyridin-3-yl)-2,3-dihydroquinazolin-4(1H)-one;
3-(2-methyl-6-oxo-1,6-dihydropyridin-3-yl)-1-phenyl-7-(trifluoromethyl)-2,3-dihydroquinazolin-4(1H)-one;
3-(2-methyl-6-oxo-1,6-dihydropyridin-3-yl)-1-(o-tolyl)-7-(trifluoromethyl)-2,3-dihydroquinazolin-4(1H)-one;
5-chloro-1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-5-methyl-3-(2-methyl-6-oxo-1,6-dihydropyridin-3-yl)-2,3-dihydroquinazolin-4(1H)-one;
3-(2-cyclopropyl-6-oxo-1,6-dihydropyridin-3-yl)-1-(4-fluoro-2-methylphenyl)-7-(trifluoromethyl)-2,3-dihydroquinazolin-4(1H)-one;
6-chloro-1-(4-fluoro-2-methoxyphenyl)-3-(2-methyl-6-oxo-1,6-dihydropyridin-3-yl)-2,3-dihydroquinazolin-4(1H)-one;
1-(4-fluoro-2-methylphenyl)-3-(2-methyl-5-oxo-2,5-dihydro-1H-pyrazol-3-yl)-7-(trifluoromethyl)-2,3-dihydroquinazolin-4(1H)-one;
7-fluoro-1-(4-fluoro-2-isopropylphenyl)-3-(2-methyl-6-oxo-1,6-dihydropyridin-3-yl)-2,3-dihydroquinazolin-4(1H)-one;
3-(4,6-dimethyl-2-oxo-1,2-dihydropyrimidin-5-yl)-1-(4-fluoro-2-methylphenyl)-7-(trifluoromethyl)-2,3-dihydroquinazolin-4(1H)-one;
1-(2-(tert-butyl)-4-fluorophenyl)-6-chloro-3-(2-methyl-6-oxo-1,6-dihydropyridin-3-yl)-2,3-dihydroquinazolin-4(1H)-one;
1-(3,4-difluoro-2-methylphenyl)-3-(2-methyl-6-oxo-1,6-dihydropyridin-3-yl)-6-(trifluoromethyl)-2,3-dihydroquinazolin-4(1H)-one;
6-chloro-1-(4-fluoro-2-isopropylphenyl)-5-methyl-3-(2-methyl-6-oxo-1,6-dihydropyridin-3-yl)-2,3-dihydroquinazolin-4(1H)-one;
6-chloro-7-fluoro-1-(4-fluoro-2-isopropylphenyl)-3-(2-methyl-6-oxo-1,6-dihydropyridin-3-yl)-2,3-dihydroquinazolin-4(1H)-one;
1-(4-fluoro-2-methylphenyl)-7-methoxy-3-(2-methyl-6-oxo-1,6-dihydropyridin-3-yl)-2,3-dihydroquinazolin-4(1H)-one;
1-(4-fluoro-2-methylphenyl)-6-hydroxy-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-6-methoxy-3-(2-methyl-6-oxo-1,6-dihydropyridin-3-yl)-7-(trifluoromethyl)-2,3-dihydroquinazolin-4(1H)-one;
6-chloro-1-(4-fluoro-2-methylphenyl)-3-(2-methyl-6-oxo-1,6-dihydropyridin-3-yl)-2,3-dihydropyrido[2,3-d]pyrimidin-4(1H)-one;
1-(4-fluoro-2-isopropylphenyl)-3-(2-methyl-6-oxo-1,6-dihydropyridin-3-yl)-7-(trifluoromethoxy)-2,3-dihydroquinazolin-4(1H)-one;
6-chloro-7-fluoro-1-(4-fluoro-2-methylphenyl)-3-(2-methyl-6-oxo-1,6-dihydropyridin-3-yl)-2,3-dihydroquinazolin-4(1H)-one;
6-chloro-1-(4-fluoro-2-methylphenyl)-3-(2-methyl-6-oxo-1,6-dihydropyridin-3-yl)-4-oxo-1,2,3,4-tetrahydroquinazoline-7-carbonitrile;
6-chloro-3-(2-ethyl-6-oxo-1,6-dihydropyridin-3-yl)-1-(4-fluoro-2-methylphenyl)-2,3-dihydroquinazolin-4(1H)-one;
7-fluoro-1-(4-fluoro-2-methylphenyl)-3-(2-methyl-6-oxo-1,6-dihydropyridin-3-yl)-6-(trifluoromethyl)-2,3-dihydroquinazolin-4(1H)-one;
5-fluoro-1-(4-fluoro-2-methylphenyl)-3-(2-methyl-6-oxo-1,6-dihydropyridin-3-yl)-6-(trifluoromethyl)-2,3-dihydroquinazolin-4(1H)-one;
7-chloro-6-fluoro-1-(4-fluoro-2-methylphenyl)-3-(2-methyl-6-oxo-1,6-dihydropyridin-3-yl)-2,3-dihydroquinazolin-4(1H)-one;
6-fluoro-1-(4-fluoro-2-methylphenyl)-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-3-(2-methyl-6-oxo-1,6-dihydropyridin-3-yl)-4-oxo-6-(trifluoromethyl)-1,2,3,4-tetrahydroquinazoline-7-carbonitrile;
1-(4-fluoro-2-methylphenyl)-3-(2-methyl-6-oxo-1,6-dihydropyridin-3-yl)-4-oxo-7-(trifluoromethoxy)-1,2,3,4-tetrahydroquinazoline-6-carbonitrile;
1-(4,5-difluoro-2-methylphenyl)-3-(2-methyl-6-oxo-1,6-dihydropyridin-3-yl)-6-(trifluoromethyl)-2,3-dihydroquinazolin-4(1H)-one;
1-(2-(tert-butyl)-4-fluorophenyl)-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-3-(6-methyl-2-oxo-1,2-dihydropyrimidin-5-yl)-7-(trifluoromethyl)-2,3-dihydroquinazolin-4(1H)-one;
1-(2-ethyl-4-fluorophenyl)-6-fluoro-3-(2-methyl-6-oxo-1,6-dihydropyridin-3-yl)-2,3-dihydroquinazolin-4(1H)-one;
4-(6-chloro-3-(2-methyl-6-oxo-1,6-dihydropyridin-3-yl)-4-oxo-3,4-dihydroquinazolin-1(2H)-yl)-3-methylbenzonitrile;
1-(5-fluoro-2-methylphenyl)-3-(2-methyl-6-oxo-1,6-dihydropyridin-3-yl)-6-(trifluoromethyl)-2,3-dihydroquinazolin-4(1H)-one;
1-(2-methyl-4-(trifluoromethoxy)phenyl)-3-(6-oxo-1,6-dihydropyridin-3-yl)-6-(trifluoromethyl)-2,3-dihydropyrido[2,3-d]pyrimidin-4(1H)-one;
1-(3-fluoro-2-methylphenyl)-3-(2-methyl-6-oxo-1,6-dihydropyridin-3-yl)-6-(trifluoromethyl)-2,3-dihydroquinazolin-4(1H)-one;
1-(2,4-difluoro-6-methylphenyl)-3-(2-methyl-6-oxo-1,6-dihydropyridin-3-yl)-6-(trifluoromethyl)-2,3-dihydroquinazolin-4(1H)-one;
7-(difluoromethyl)-1-(4-fluoro-2-isopropylphenyl)-3-(2-methyl-6-oxo-1,6-dihydropyridin-3-yl)-2,3-dihydropyrido[2,3-d]pyrimidin-4(1H)-one;
3-methyl-4-(3-(2-methyl-6-oxo-1,6-dihydropyridin-3-yl)-4-oxo-6-(trifluoromethoxy)-3,4-dihydroquinazolin-1(2H)-yl)benzonitrile;
8-chloro-1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-8-methyl-3-(2-methyl-6-oxo-1,6-dihydropyridin-3-yl)-2,3-dihydroquinazolin-4(1H)-one;
1-(4-methoxy-2-methylphenyl)-3-(2-methyl-6-oxo-1,6-dihydropyridin-3-yl)-6-(trifluoromethyl)-2,3-dihydroquinazolin-4(1H)-one;
1-(4-hydroxy-2-methylphenyl)-3-(2-methyl-6-oxo-1,6-dihydropyridin-3-yl)-6-(trifluoromethyl)-2,3-dihydroquinazolin-4(1H)-one;
3-(6-chloro-3-(2-methyl-6-oxo-1,6-dihydropyridin-3-yl)-4-oxo-3,4-dihydroquinazolin-1(2H)-yl)-2-methylbenzonitrile;
6-fluoro-1-(4-fluoro-2-methylphenyl)-3-(2-methyl-6-oxo-1,6-dihydropyridin-3-yl)-7-(trifluoromethoxy)-2,3-dihydroquinazolin-4(1H)-one;
5-(difluoromethyl)-1-(4-fluoro-2-methylphenyl)-3-(2-methyl-6-oxo-1,6-dihydropyridin-3-yl)-2,3-dihydroquinazolin-4(1H)-one;
3-(5-fluoro-2-methyl-6-oxo-1,6-dihydropyridin-3-yl)-1-(4-fluoro-2-methylphenyl)-6-(trifluoromethyl)-2,3-dihydroquinazolin-4(1H)-one;
6,7-difluoro-1-(4-fluoro-2-methoxyphenyl)-3-(2-methyl-6-oxo-1,6-dihydropyridin-3-yl)-2,3-dihydroquinazolin-4(1H)-one;
6-chloro-1-(2-hydroxy-4-methylphenyl)-3-(2-methyl-6-oxo-1,6-dihydropyridin-3-yl)-2,3-dihydroquinazolin-4(1H)-one;
6-chloro-1-(2-methoxy-4-methylphenyl)-3-(2-methyl-6-oxo-1,6-dihydropyridin-3-yl)-2,3-dihydroquinazolin-4(1H)-one;
1-(2-fluoro-6-methylphenyl)-3-(2-methyl-6-oxo-1,6-dihydropyridin-3-yl)-6-(trifluoromethyl)-2,3-dihydroquinazolin-4(1H)-one;
6-chloro-5-fluoro-1-(4-fluoro-2-methylphenyl)-3-(2-oxo-1,2-dihydropyridin-4-yl)-2,3-dihydroquinazolin-4(1H)-one;
7-(difluoromethyl)-1-(4-fluoro-2-isopropylphenyl)-3-(2-methyl-6-oxo-1,6-dihydropyridin-3-yl)-2,3-dihydroquinazolin-4(1H)-one;
7-(difluoromethyl)-1-(4-fluoro-2-methylphenyl)-3-(2-methyl-6-oxo-1,6-dihydropyridin-3-yl)-2,3-dihydroquinazolin-4(1H)-one;
6-chloro-3-(2-ethyl-6-oxo-1,6-dihydropyridin-3-yl)-1-(4-fluoro-2-isopropylphenyl)-2,3-dihydroquinazolin-4(1H)-one;
6-fluoro-2-methyl-3-(3-(2-methyl-6-oxo-1,6-dihydropyridin-3-yl)-4-oxo-6-(trifluoromethyl)-3,4-dihydroquinazolin-1(2H)-yl)benzonitrile;
6-fluoro-1-(4-fluoro-2-isopropylphenyl)-3-(2-methyl-6-oxo-1,6-dihydropyridin-3-yl)-2,3-dihydropyrido[2,3-d]pyrimidin-4(1H)-one;
1-(2-bromo-4-fluorophenyl)-3-(2-methyl-6-oxo-1,6-dihydropyridin-3-yl)-6-(trifluoromethyl)-2,3-dihydroquinazolin-4(1H)-one;
6-fluoro-1-(4-fluoro-2-methylphenyl)-3-(2-methyl-6-oxo-1,6-dihydropyridin-3-yl)-2,3-dihydroquinazolin-4(1H)-one;
1-(4-Fluorophenyl)-3-(6-oxo-1,6-dihydropyridin-3-yl)-7-(trifluoromethyl)-2,3-dihydroquinazolin-4(1H)-one;
1-(4-fluoro-2-methylphenyl)-3-(2-oxo-1,2-dihydropyrimidin-5-yl)-7-(trifluoromethyl)-2,3-dihydroquinazolin-4(1H)-one;
1-(4-bromo-2-methylphenyl)-3-(6-oxo-1,6-dihydropyridin-3-yl)-7-(trifluoromethyl)-2,3-dihydroquinazolin-4(1H)-one;
1-(4-fluorophenyl)-3-(2-methyl-6-oxo-1,6-dihydropyridin-3-yl)-7-(trifluoromethyl)-2,3-dihydroquinazolin-4(1H)-one;
3-methyl-4-(3-(2-methyl-6-oxo-1,6-dihydropyridin-3-yl)-4-oxo-7-(trifluoromethyl)-3,4-dihydroquinazolin-1(2H)-yl)benzonitrile;
1-(4-fluoro-2-methylphenyl)-3-(2-methyl-6-oxo-1,6-dihydropyridin-3-yl)-7-(trifluoromethyl)-2,3-dihydropyrido[2,3-d]pyrimidin-4(1H)-one;
3-(2-ethyl-6-oxo-1,6-dihydropyridin-3-yl)-1-(4-fluoro-2-methylphenyl)-7-(trifluoromethyl)-2,3-dihydroquinazolin-4(1H)-one;
3-(2-chloro-6-oxo-1,6-dihydropyridin-3-yl)-1-(4-fluoro-2-methylphenyl)-7-(trifluoromethyl)-2,3-dihydroquinazolin-4(1H)-one;
7-bromo-1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-3-(2-methyl-6-oxo-1,6-dihydropyridin-3-yl)-4-oxo-1,2,3,4-tetrahydroquinazoline-7-carbonitrile;
3-(1-(4-fluoro-2-methylphenyl)-4-oxo-7-(trifluoromethyl)-1,4-dihydroquinazolin-3(2H)-yl)-6-oxo-1,6-dihydropyridine-2-carbonitrile;
1-(2,4-difluorophenyl)-3-(2-methyl-6-oxo-1,6-dihydropyridin-3-yl)-7-(trifluoromethyl)-2,3-dihydroquinazolin-4(1H)-one;
1-(4-ethoxyphenyl)-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-3-(2-oxo-1,2-dihydropyridin-4-yl)-7-(trifluoromethyl)-2,3-dihydroquinazolin-4(1H)-one;
6,7-difluoro-1-(4-fluoro-2-methylphenyl)-3-(2-methyl-6-oxo-1,6-dihydropyridin-3-yl)-2,3-dihydroquinazolin-4(1H)-one;
6,7-difluoro-1-(4-fluoro-2-isopropylphenyl)-3-(2-methyl-6-oxo-1,6-dihydropyridin-3-yl)-2,3-dihydroquinazolin-4(1H)-one;
3-(2-methyl-6-oxo-1,6-dihydropyridin-3-yl)-1-((1S,2R)-2-methylcyclohexyl)-7-(trifluoromethyl)-2,3-dihydroquinazolin-4(1H)-one;
1-(4-fluoro-2-isopropylphenyl)-3-(2-methyl-6-oxo-1,6-dihydropyridin-3-yl)-4-oxo-1,2,3,4-tetrahydroquinazoline-7-carbonitrile;
3-(2-methyl-6-oxo-1,6-dihydropyridin-3-yl)-1-((1R,2S)-2-methylcyclohexyl)-7-(trifluoromethyl)-2,3-dihydroquinazolin-4(1H)-one;
N-(3-(1-(4-fluoro-2-methylphenyl)-4-oxo-6-(trifluoromethyl)-1,4-dihydroquinazolin-3(2H)-yl)-6-oxo-1,6-dihydropyridin-2-yl)acetamide;
3-(2-bromo-6-oxo-1,6-dihydropyridin-3-yl)-1-(4-fluoro-2-methylphenyl)-6-(trifluoromethyl)-2,3-dihydroquinazolin-4(1H)-one;
6-fluoro-1-(4-fluoro-2-methylphenyl)-7-methoxy-3-(2-methyl-6-oxo-1,6-dihydropyridin-3-yl)-2,3-dihydropyrido[2,3-d]pyrimidin-4(1H)-one;
1-(4-fluoro-2-methylphenyl)-3-(2-methyl-6-oxo-1,6-dihydropyridin-3-yl)-4-oxo-7-(trifluoromethyl)-1,2,3,4-tetrahydroquinazoline-6-carbonitrile;
6-chloro-7-(difluoromethoxy)-1-(4-fluoro-2-methylphenyl)-3-(2-methyl-6-oxo-1,6-dihydropyridin-3-yl)-2,3-dihydroquinazolin-4(1H)-one;
6-chloro-7-(difluoromethyl)-1-(4-fluoro-2-methylphenyl)-3-(2-methyl-6-oxo-1,6-dihydropyridin-3-yl)-2,3-dihydroquinazolin-4(1H)-one;
3-(4-amino-2-oxo-1,2-dihydropyrimidin-5-yl)-1-(4-fluoro-2-methylphenyl)-6-(trifluoromethyl)-2,3-dihydroquinazolin-4(1H)-one;
6-chloro-1-(2-methyl-4-(trifluoromethoxy)phenyl)-3-(2-methyl-6-oxo-1,6-dihydropyridin-3-yl)-4-oxo-1,2,3,4-tetrahydropyrido[2,3-d]pyrimidine-7-carbonitrile;
6-chloro-1-(4-fluoro-2-methylphenyl)-3-(2-methyl-6-oxo-1,6-dihydropyridin-3-yl)-4-oxo-1,2,3,4-tetrahydropyrido[2,3-d]pyrimidine-7-carbonitrile;
6-chloro-1-(2-methyl-4-(trifluoromethoxy)phenyl)-3-(2-methyl-6-oxo-1,6-dihydropyridin-3-yl)-4-oxo-1,2,3,4-tetrahydroquinazoline-7-carbonitrile;
6-chloro-1-(4-fluoro-2-methylphenyl)-3-(6-methyl-2-oxo-1,2-dihydropyrimidin-5-yl)-4-oxo-1,2,3,4-tetrahydroquinazoline-7-carbonitrile;
1-(2-methyl-3-(trifluoromethyl)phenyl)-3-(2-methyl-6-oxo-1,6-dihydropyridin-3-yl)-6-(trifluoromethyl)-2,3-dihydropyrido[2,3-d]pyrimidin-4(1H)-one;
1-(3-chloro-2-methylphenyl)-3-(2-methyl-6-oxo-1,6-dihydropyridin-3-yl)-6-(trifluoromethyl)-2,3-dihydropyrido[2,3-d]pyrimidin-4(1H)-one;
1-(2-ethyl-4-fluorophenyl)-3-(2-methyl-6-oxo-1,6-dihydropyridin-3-yl)-6-(trifluoromethyl)-2,3-dihydropyrido[2,3-d]pyrimidin-4(1H)-one;
1-(3,4-difluoro-2-methylphenyl)-3-(2-methyl-6-oxo-1,6-dihydropyridin-3-yl)-6-(trifluoromethyl)-2,3-dihydropyrido[2,3-d]pyrimidin-4(1H)-one;
1-(4-fluoro-2-isopropylphenyl)-3-(2-methyl-6-oxo-1,6-dihydropyridin-3-yl)-6-(trifluoromethyl)-2,3-dihydroquinazolin-4(1H)-one;
1-(4-fluoro-2-methylphenyl)-3-(4-methyl-6-oxo-1,6-dihydropyridin-3-yl)-6-(trifluoromethyl)-2,3-dihydroquinazolin-4(1H)-one;
6-fluoro-1-(4-fluoro-2-isopropylphenyl)-3-(2-methyl-6-oxo-1,6-dihydropyridin-3-yl)-2,3-dihydroquinazolin-4(1H)-one;
1-(2-cyclopropyl-4-fluorophenyl)-3-(2-methyl-6-oxo-1,6-dihydropyridin-3-yl)-2,3-dihydroquinazolin-4(1H)-one;
1-(2-cyclopropyl-4-fluorophenyl)-3-(2-methyl-6-oxo-1,6-dihydropyridin-3-yl)-7-(trifluoromethyl)-2,3-dihydroquinazolin-4(1H)-one;
5-fluoro-1-(4-fluoro-2-methylphenyl)-3-(2-methyl-6-oxo-1,6-dihydropyridin-3-yl)-2,3-dihydroquinazolin-4(1H)-one;
1-(2-ethyl-4-fluorophenyl)-3-(2-methyl-6-oxo-1,6-dihydropyridin-3-yl)-7-(trifluoromethyl)-2,3-dihydroquinazolin-4(1H)-one;
7-(difluoromethoxy)-1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-3-(6-oxo-1,6-dihydropyridin-3-yl)-7-(trifluoromethyl)quinazoline-2,4(1H,3H)-dione;
7-cyclopropyl-1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-3-(2-methyl-6-oxo-1,6-dihydropyridin-3-yl)-6-(trifluoromethoxy)-2,3-dihydroquinazolin-4(1H)-one;
1-(4-(difluoromethoxy)-2-methylphenyl)-3-(2-methyl-6-oxo-1,6-dihydropyridin-3-yl)-6-(trifluoromethyl)-2,3-dihydroquinazolin-4(1H)-one;
3-(2-ethyl-6-oxo-1,6-dihydropyridin-3-yl)-1-(4-fluoro-2-methylphenyl)-7-(trifluoromethoxy)-2,3-dihydroquinazolin-4(1H)-one;
6-chloro-1-(2-ethyl-4-fluorophenyl)-7-fluoro-3-(2-methyl-6-oxo-1,6-dihydropyridin-3-yl)-2,3-dihydroquinazolin-4(1H)-one;
6-chloro-1-(2-ethyl-4-fluorophenyl)-3-(2-methyl-6-oxo-1,6-dihydropyridin-3-yl)-4-oxo-1,2,3,4-tetrahydroquinazoline-7-carbonitrile;
6-chloro-5,7-difluoro-1-(4-fluoro-2-methylphenyl)-3-(2-methyl-6-oxo-1,6-dihydropyridin-3-yl)-2,3-dihydroquinazolin-4(1H)-one;
6-chloro-5-fluoro-1-(4-fluoro-2-methylphenyl)-3-(2-methyl-6-oxo-1,6-dihydropyridin-3-yl)-4-oxo-1,2,3,4-tetrahydroquinazoline-7-carbonitrile;
1-(2-methyl-4-(trifluoromethyl)phenyl)-3-(2-methyl-6-oxo-1,6-dihydropyridin-3-yl)-6-(trifluoromethyl)-2,3-dihydropyrido[2,3-d]pyrimidin-4(1H)-one;
1-(4-chloro-2-methylphenyl)-3-(2-methyl-6-oxo-1,6-dihydropyridin-3-yl)-6-(trifluoromethyl)-2,3-dihydropyrido[2,3-d]pyrimidin-4(1H)-one;
7-(1,1-difluoro-2-hydroxyethyl)-1-(4-fluoro-2-methylphenyl)-3-(2-methyl-6-oxo-1,6-dihydropyridin-3-yl)-2,3-dihydroquinazolin-4(1H)-one;
1-(4-fluoro-2-methylphenyl)-3-(2-isopropyl-6-oxo-1,6-dihydropyridin-3-yl)-7-(trifluoromethyl)-2,3-dihydroquinazolin-4(1H)-one;
1-(4-fluoro-2-methylphenyl)-3-(2-((2-hydroxyethyl)amino)-6-oxo-1,6-dihydropyridin-3-yl)-7-(trifluoromethyl)-2,3-dihydroquinazolin-4(1H)-one;
6-chloro-1-(2-(dimethylamino)-4-fluorophenyl)-3-(2-methyl-6-oxo-1,6-dihydropyridin-3-yl)-2,3-dihydroquinazolin-4(1H)-one;
6-chloro-1-(4-fluoro-2-(methylamino)phenyl)-3-(2-methyl-6-oxo-1,6-dihydropyridin-3-yl)-2,3-dihydroquinazolin-4(1H)-one;
1-(4-(1,1-difluoro-2-hydroxyethyl)-2-methylphenyl)-3-(2-methyl-6-oxo-1,6-dihydropyridin-3-yl)-7-(trifluoromethyl)-2,3-dihydroquinazolin-4(1H)-one;
1-(2,4-dimethylphenyl)-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-3-(2-oxo-1,2-dihydropyridin-3-yl)-7-(trifluoromethyl)-2,3-dihydroquinazolin-4(1H)-one;
1-(4-fluoro-2-methylphenyl)-3-(6-methyl-2-oxo-1,2-dihydropyrimidin-5-yl)-7-(trifluoromethoxy)-2,3-dihydroquinazolin-4(1H)-one;
1-(4-fluoro-2-methylphenyl)-7-methyl-3-(2-methyl-6-oxo-1,6-dihydropyridin-3-yl)-2,3-dihydropyrido[2,3-d]pyrimidin-4(1H)-one;
6-chloro-1-(2-ethyl-4-fluorophenyl)-3-(2-methyl-6-oxo-1,6-dihydropyridin-3-yl)-4-oxo-1,2,3,4-tetrahydropyrido[2,3-d]pyrimidine-7-carbonitrile;
1-((1S,3S)-3-fluorocyclopentyl)-3-(2-methyl-6-oxo-1,6-dihydropyridin-3-yl)-7-(trifluoromethyl)-2,3-dihydroquinazolin-4(1H)-one;
1-((1R,3R)-3-fluorocyclopentyl)-3-(2-methyl-6-oxo-1,6-dihydropyridin-3-yl)-7-(trifluoromethyl)-2,3-dihydroquinazolin-4(1H)-one;
3-(1,2-dimethyl-6-oxo-1,6-dihydropyridin-3-yl)-1-(4-fluoro-2-methylphenyl)-6-(trifluoromethyl)-2,3-dihydroquinazolin-4(1H)-one;
3-(2-chloro-6-oxo-1,6-dihydropyridin-3-yl)-1-(4-fluoro-2-methylphenyl)-6-(trifluoromethyl)-2,3-dihydroquinazolin-4(1H)-one;
3-(2-bromo-4-methyl-6-oxo-1,6-dihydropyridin-3-yl)-1-(4-fluoro-2-methylphenyl)-7-(trifluoromethyl)-2,3-dihydroquinazolin-4(1H)-one;
1-(4-fluoro-2-isopropylphenyl)-3-(2-methyl-6-oxo-1,6-dihydropyridin-3-yl)-7-(trifluoromethyl)-2,3-dihydropyrido[2,3-d]pyrimidin-4(1H)-one;
6-chloro-1-(4-fluoro-2-isopropylphenyl)-3-(2-methyl-6-oxo-1,6-dihydropyridin-3-yl)-2,3-dihydropyrido[2,3-d]pyrimidin-4(1H)-one;
5-(1-(4-fluoro-2-methylphenyl)-4-thioxo-7-(trifluoromethyl)-1,4-dihydroquinazolin-3(2H)-yl)-6-methylpyridin-2(1H)-one;
1-(4-Fluoro-2-methylphenyl)-3-(6-oxo-1,6-dihydropyridin-3-yl)-6-(trifluoromethyl)-2,3-dihydroquinazolin-4(1H)-one;
1-(4-fluoro-2-methoxyphenyl)-3-(6-oxo-1,6-dihydropyridin-3-yl)-7-(trifluoromethyl)-2,3-dihydroquinazolin-4(1H)-one;
1-(4-fluoro-2-methylphenyl)-3-(6-oxo-1,6-dihydropyridin-3-yl)-5-(trifluoromethyl)-2,3-dihydroquinazolin-4(1H)-one;
1-(4-fluoro-2-methylphenyl)-3-(5-methyl-6-oxo-1,6-dihydropyridin-3-yl)-7-(trifluoromethyl)-2,3-dihydroquinazolin-4(1H)-one;
1-(4-fluoro-2-methylphenyl)-3-(6-oxo-1,6-dihydropyridazin-3-yl)-7-(trifluoromethyl)-2,3-dihydroquinazolin-4(1H)-one;
1-(4-fluorobenzyl)-3-(6-oxo-1,6-dihydropyridin-3-yl)-7-(trifluoromethyl)-2,3-dihydroquinazolin-4(1H)-one;
1-(4-fluoro-2-methylphenyl)-3-(4-methyl-6-oxo-1,6-dihydropyridin-3-yl)-7-(trifluoromethyl)-2,3-dihydroquinazolin-4(1H)-one;
1-(4-fluoro-2-methylphenyl)-4-oxo-3-(6-oxo-1,6-dihydropyridin-3-yl)-1,2,3,4-tetrahydroquinazoline-7-carbonitrile;
1-(4-fluoro-2-methoxyphenyl)-3-(6-oxo-1,6-dihydropyridin-3-yl)-5-(trifluoromethyl)-2,3-dihydroquinazolin-4(1H)-one;
3-(2-methyl-6-oxo-1,6-dihydropyridin-3-yl)-1-(4-methylthiazol-5-yl)-6-(trifluoromethyl)-2,3-dihydroquinazolin-4(1H)-one;
1-(2,4-dimethoxyphenyl)-3-(2-methyl-6-oxo-1,6-dihydropyridin-3-yl)-6-(trifluoromethyl)-2,3-dihydroquinazolin-4(1H)-one;
1-(4-fluoro-2-methoxyphenyl)-3-(2-methyl-6-oxo-1,6-dihydropyridin-3-yl)-6-(trifluoromethyl)-2,3-dihydroquinazolin-4(1H)-one;
7-chloro-6-fluoro-1-(4-fluoro-2-methylphenyl)-3-(2-methyl-6-oxo-1,6-dihydropyridin-3-yl)-2,3-dihydropyrido[2,3-d]pyrimidin-4(1H)-one;
3-(2-methyl-6-oxo-1,6-dihydropyridin-3-yl)-1-(3-methylthiophen-2-yl)-6-(trifluoromethyl)-2,3-dihydroquinazolin-4(1H)-one;
1-(4-fluoro-2-methoxyphenyl)-4-oxo-3-(6-oxo-1,6-dihydropyridin-3-yl)-1,2,3,4-tetrahydroquinazoline-7-carbonitrile;
1-(4-fluoro-2-methylphenyl)-3-(2-methyl-6-oxo-1,6-dihydropyridin-3-yl)-7-(2,2,2-trifluoroethoxy)-2,3-dihydroquinazolin-4(1H)-one;
1-(2-methyl-4-(trifluoromethoxy)phenyl)-3-(6-oxo-1,6-dihydropyridin-3-yl)-6-(trifluoromethyl)-2,3-dihydroquinazolin-4(1H)-one;
6-chloro-3-(4-chloro-2-methyl-6-oxo-1,6-dihydropyridin-3-yl)-1-(4-fluoro-2-methylphenyl)-2,3-dihydroquinazolin-4(1H)-one;
7-chloro-1-(4-fluoro-2-methylphenyl)-3-(2-methyl-6-oxo-1,6-dihydropyridin-3-yl)-4-oxo-1,2,3,4-tetrahydroquinazoline-6-carbonitrile;
6-chloro-1-(4-fluoro-2-methylphenyl)-5-methyl-3-(2-methyl-6-oxo-1,6-dihydropyridin-3-yl)-2,3-dihydroquinazolin-4(1H)-one;
1-(3,5-difluoro-2-methylphenyl)-3-(2-methyl-6-oxo-1,6-dihydropyridin-3-yl)-6-(trifluoromethyl)-2,3-dihydroquinazolin-4(1H)-one;
1-(4-fluoro-2-isopropylphenyl)-5-methoxy-3-(2-methyl-6-oxo-1,6-dihydropyridin-3-yl)-2,3-dihydroquinazolin-4(1H)-one;
1-(bicyclo[1.1.1]pentan-1-yl)-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-3-(3-methyl-2-oxo-1,2-dihydropyridin-4-yl)-6-(trifluoromethyl)-2,3-dihydroquinazolin-4(1H)-one;
5-(1-(4-fluoro-2-methylphenyl)-6-(trifluoromethyl)-1,4-dihydroquinazolin-3(2H)-yl)-6-methylpyridin-2(1H)-one;
3-(2-Methyl-6-oxo-1,6-dihydropyridin-3-yl)-1-(2-(2,2,2-trifluoroethyl)phenyl)-6-(trifluoromethyl)-2,3-dihydroquinazolin-4(1H)-one;
7-(difluoromethoxy)-1-(4-fluoro-2-methylphenyl)-3-(2-methyl-6-oxo-1,6-dihydropyridin-3-yl)-2,3-dihydropyrido[2,3-d]pyrimidin-4(1H)-one;
6-fluoro-1-(4-fluoro-2-methylphenyl)-3-(2-methyl-6-oxo-1,6-dihydropyridin-3-yl)-4-oxo-1,2,3,4-tetrahydroquinazoline-7-carbonitrile;
6-chloro-1-(2-ethyl-4-fluorophenyl)-3-(3-methylpyridin-4-yl)-4-oxo-1,2,3,4-tetrahydroquinazoline-7-carbonitrile;
4-(6-Chloro-7-(difluoromethyl)-1-(4-fluoro-2-methylphenyl)-4-oxo-1,4-dihydroquinazolin-3(2H)-yl)-3-methylpyridine 1-oxide;
4-(6-chloro-1-(2-ethyl-4-fluorophenyl)-7-fluoro-4-oxo-1,4-dihydroquinazolin-3(2H)-yl)-3-methylpyridine 1-oxide;
4-(6-chloro-7-fluoro-1-(2-methyl-4-(trifluoromethoxy)phenyl)-4-oxo-1,4-dihydroquinazolin-3(2H)-yl)-3-methylpyridine 1-oxide;
4-(6-chloro-7-cyano-1-(2-ethyl-4-fluorophenyl)-4-oxo-1,4-dihydroquinazolin-3(2H)-yl)-3-methylpyridine 1-oxide;
2-carbamoyl-5-(6-chloro-5-fluoro-1-(4-fluoro-2-methylphenyl)-4-oxo-1,4-dihydroquinazolin-3(2H)-yl)pyridine 1-oxide;
3-(1-(4-fluoro-2-methylphenyl)-4-oxo-6-(trifluoromethyl)-1,4-dihydroquinazolin-3(2H)-yl)pyridine 1-oxide;
3-(1-(4-fluoro-2-methylphenyl)-4-oxo-6-(trifluoromethyl)-1,4-dihydroquinazolin-3(2H)-yl)-4-methylpyridine 1-oxide;
5-(1-(4-fluoro-2-methylphenyl)-4-oxo-6-(trifluoromethyl)-1,4-dihydroquinazolin-3(2H)-yl)pyridazine 1-oxide;
4-(1-(4-fluoro-2-methylphenyl)-4-oxo-6-(trifluoromethyl)-1,4-dihydroquinazolin-3(2H)-yl)pyridazine 1-oxide;
3-(1-(4-fluoro-2-methylphenyl)-4-oxo-6-(trifluoromethyl)-1,4-dihydroquinazolin-3(2H)-yl)-2-methylpyridine 1-oxide;
4-(1-(4-fluoro-2-methylphenyl)-4-oxo-6-(trifluoromethyl)-1,4-dihydroquinazolin-3(2H)-yl)-3-methylpyridine 1-oxide;
3-(1-(4-fluoro-2-methylphenyl)-4-oxo-6-(trifluoromethyl)-1,4-dihydroquinazolin-3(2H)-yl)-5-methylpyridine 1-oxide;
3-methyl-4-(1-(2-methyl-4-(trifluoromethoxy)phenyl)-4-oxo-6-(trifluoromethyl)-1,4-dihydroquinazolin-3(2H)-yl)pyridine 1-oxide;
3-(2-Amino-6-oxo-1,6-dihydropyridin-3-yl)-1-(4-fluoro-2-methylphenyl)-6-(trifluoromethyl)-2,3-dihydroquinazolin-4(1H)-one;
3-(6-Amino-2-methylpyridin-3-yl)-1-(4-fluoro-2-methylphenyl)-6-(trifluoromethyl)-2,3-dihydroquinazolin-4(1H)-one;
2-((5-(6-Chloro-1-(4-fluoro-2-methylphenyl)-4-oxo-1,4-dihydroquinazolin-3(2H)-yl)-6-methylpyridin-2-yl)oxy)acetic acid;
1-(4-Fluoro-2-methylphenyl)-3-(2-methoxy-6-oxo-1,6-dihydropyridin-3-yl)-7-(trifluoromethyl)-2,3-dihydroquinazolin-4(1H)-one;
3-(6-Aminopyridin-3-yl)-1-(4-fluoro-2-methylphenyl)-7-(trifluoromethyl)-2,3-dihydroquinazolin-4(1H)-one;
4-(1-(4-Fluoro-2-methylphenyl)-4-oxo-7-(trifluoromethyl)-1,4-dihydroquinazolin-3(2H)-yl)benzoic acid;
3-(1-(4-fluoro-2-methylphenyl)-4-oxo-7-(trifluoromethyl)-1,4-dihydroquinazolin-3(2H)-yl)benzoic acid;
1-(4-Fluoro-2-methylphenyl)-3-(2-hydroxy-6-oxo-1,6-dihydropyridin-3-yl)-7-(trifluoromethyl)-2,3-dihydroquinazolin-4(1H)-one;
1-(4-Fluoro-2-methylphenyl)-3-(2-methyl-6-oxo-1,6-dihydropyridin-3-yl)-7-(oxetan-3-yl)-2,3-dihydroquinazolin-4(1H)-one;
1-(4-Fluoro-2-methylphenyl)-3-(4-hydroxy-2-methylphenyl)-6-(trifluoromethyl)-2,3-dihydroquinazolin-4(1H)-one;
1-(4-Fluoro-2-methylphenyl)-3-(4-hydroxy-2-methylphenyl)-7-(trifluoromethyl)-2,3-dihydroquinazolin-4(1H)-one;
1-(4-Fluorophenyl)-3-(6-oxo-1,6-dihydropyridin-3-yl)-7-(trifluoromethyl)-2,3-dihydroquinazolin-4(1H)-one;
1-(4-fluoro-2-methylphenyl)-3-(2-methyl-6-oxo-1,4,5,6-tetrahydropyridin-3-yl)-7-(trifluoromethyl)-2,3-dihydroquinazolin-4(1H)-one;
1-(4-fluoro-2-methylphenyl)-3-(2-oxohexahydropyrimidin-5-yl)-7-(trifluoromethyl)-2,3-dihydroquinazolin-4(1H)-one;
1-(4-fluoro-2-methylphenyl)-3-(6-methyl-2-oxo-1,2,3,4-tetrahydropyrimidin-5-yl)-7-(trifluoromethyl)-2,3-dihydroquinazolin-4(1H)-one;
3-(1-(4-Fluoro-2-methylphenyl)-4-oxo-7-(trifluoromethyl)-1,4-dihydroquinazolin-3(2H)-yl)benzamide;
3-(6-chloro-1-(4-fluoro-2-methylphenyl)-4-oxo-1,4-dihydroquinazolin-3(2H)-yl)furan-2-carboxamide;
4-Methyl-3-(3-(2-methyl-6-oxo-1,6-dihydropyridin-3-yl)-4-oxo-6-(trifluoromethyl)-3,4-dihydroquinazolin-1(2H)-yl)benzamide;
4-(6-Chloro-1-(4-fluoro-2-methylphenyl)-4-oxo-1,4-dihydroquinazolin-3(2H)-yl)furan-2-carboxamide;
3-(1-(4-Fluoro-2-methylphenyl)-4-oxo-7-(trifluoromethyl)-1,4-dihydroquinazolin-3(2H)-yl)benzenesulfonamide;
3-(6-Amino-4-methylpyridin-3-yl)-1-(4-fluoro-2-methylphenyl)-6-(trifluoromethyl)-2,3-dihydroquinazolin-4(1H)-one;
3-(2-Methyl-6-oxo-1,6-dihydropyridin-3-yl)-1-((1S,2S)-2-methylcyclohexyl)-7-(trifluoromethyl)-2,3-dihydroquinazolin-4(1H)-one;
3-(2-Methyl-6-oxo-1,6-dihydropyridin-3-yl)-1-((1R,2R)-2-methylcyclohexyl)-7-(trifluoromethyl)-2,3-dihydroquinazolin-4(1H)-one;
3-(2-Methyl-6-oxo-1,6-dihydropyridin-3-yl)-1-((3S,4S)-3-methyltetrahydro-2H-pyran-4-yl)-7-(trifluoromethyl)-2,3-dihydroquinazolin-4(1H)-one;
3-(2-Methyl-6-oxo-1,6-dihydropyridin-3-yl)-1-((3R,4R)-3-methyltetrahydro-2H-pyran-4-yl)-7-(trifluoromethyl)-2,3-dihydroquinazolin-4(1H)-one;
3-(2-Methyl-6-oxo-1,6-dihydropyridin-3-yl)-1-((3R,4S)-3-methyltetrahydro-2H-pyran-4-yl)-7-(trifluoromethyl)-2,3-dihydroquinazolin-4(1H)-one;
3-(2-Methyl-6-oxo-1,6-dihydropyridin-3-yl)-1-((3S,4R)-3-methyltetrahydro-2H-pyran-4-yl)-7-(trifluoromethyl)-2,3-dihydroquinazolin-4(1H)-one;
rel-(R)-1-(4-fluoro-2-methylphenyl)-2-methyl-3-(6-oxo-1,6-dihydropyridin-3-yl)-7-(trifluoromethyl)-2,3-dihydroquinazolin-4(1H)-one;
rel-(R)-1-(4-fluoro-2-methylphenyl)-2-methyl-3-(6-oxo-1,6-dihydropyridin-3-yl)-7-(trifluoromethyl)-2,3-dihydroquinazolin-4(1H)-one;
1-(4-Fluoro-2-methylphenyl)-3-((2S,3S)-2-methyl-6-oxopiperidin-3-yl)-7-(trifluoromethyl)-2,3-dihydroquinazolin-4(1H)-one;
1-(4-Fluoro-2-methylphenyl)-3-((2R,3R)-2-methyl-6-oxopiperidin-3-yl)-7-(trifluoromethyl)-2,3-dihydroquinazolin-4(1H)-one;
3-(2,4-Dimethyl-6-oxo-1,6-dihydropyridin-3-yl)-1-(4-fluoro-2-isopropylphenyl)-7-(trifluoromethyl)-2,3-dihydroquinazolin-4(1H)-one;
3-(2,4-dimethyl-6-oxo-1,6-dihydropyridin-3-yl)-1-(4-fluoro-2-isopropylphenyl)-7-(trifluoromethyl)-2,3-dihydroquinazolin-4(1H)-one;
6-Chloro-3-(2,4-dimethyl-6-oxo-1,6-dihydropyridin-3-yl)-1-(4-fluoro-2-isopropylphenyl)-2,3-dihydroquinazolin-4(1H)-one;
6-Chloro-3-(2,4-dimethyl-6-oxo-1,6-dihydropyridin-3-yl)-1-(4-fluoro-2-isopropylphenyl)-2,3-dihydroquinazolin-4(1H)-one;
1-(2-(tert-Butyl)-4-fluorophenyl)-6-chloro-3-(2-methyl-6-oxo-1,6-dihydropyridin-3-yl)-2,3-dihydroquinazolin-4(1H)-one;
1-(2-(tert-Butyl)-4-fluorophenyl)-6-chloro-3-(2-methyl-6-oxo-1,6-dihydropyridin-3-yl)-2,3-dihydroquinazolin-4(1H)-one;
1-(4-Fluoro-2-methylphenyl)-3-(1-methyl-6-oxo-1,6-dihydropyridin-3-yl)-7-(trifluoromethyl)-2,3-dihydro quinazolin-4(1H)-one;
1-(4-Fluoro-2-methylphenyl)-3-(1-methyl-6-oxo-1,6-dihydropyridin-3-yl)-7-(trifluoromethyl)-2,3-dihydro quinazolin-4(1H)-one;
1-(4-fluoro-2-methylphenyl)-3-(2-methyl-6-oxo-1,6-dihydropyridin-3-yl)-6-(trifluoromethyl)-2,3-dihydroquinazolin-4(1H)-one;
1-(2-methyl-4-(trifluoromethoxy)phenyl)-3-(2-methyl-6-oxo-1,6-dihydropyridin-3-yl)-6-(trifluoromethyl)-2,3-dihydropyrido[2,3-d]pyrimidin-4(1H)-one; and
1-(4-fluoro-2-methylphenyl)-3-(2-methyl-6-oxo-1,6-dihydropyridin-3-yl)-6-(trifluoromethyl)-2,3-dihydropyrido[2,3-d]pyrimidin-4(1H)-one;
or a tautomer thereof,
or a pharmaceutically acceptable salt thereof.

12. (canceled)

13. A pharmaceutical composition comprising:
a compound of Formula (X) or a pharmaceutically acceptable salt and/or a corresponding tautomer form thereof according to claim 1; and
one or more pharmaceutically acceptable excipient(s).

14. A method for treating:
pain-associated disease(s);
pain caused by trauma; or
pain caused by iatrogenic, medical or dental procedures,
comprising administering to a human in need thereof a therapeutically effective amount of:
a compound according to claim 1 or a pharmaceutically acceptable salt and/or a corresponding tautomer form thereof.

15. The method according to claim 14, wherein:
the pain-associated disease(s);
the pain caused by trauma; or
the pain caused by iatrogenic, medical or dental procedures, respectively,
is selected from:
chronic pain;
acute pain;
neuropathic pain;
inflammatory pain of varied physiologic origins;
nociceptive pain;
neurologic, neuropathy or neuronal injury associated or related pain disorders caused by diseases; neuralgias and associated acute or chronic pain;
post-herpetic neuralgia;
musculoskeletal pain; lower back and neck pain; sprains and strains;
myofascial pain; myositis or muscle inflammation;
repetitive motion pain;
complex regional pain syndrome;
chronic or acute arthritic pain;
sympathetically maintained pain;
cancer, toxins and chemotherapy related pain;
postsurgical pain syndromes and/or associated phantom limb pain;
post-operative medical or dental procedures or treatments pain; or
pain associated with HIV, pain induced by HIV treatment.

16. The method according to claim 15, wherein:
nociceptive pain is selected from post-surgical pain, cancer pain, back and craniofacial pain, osteoarthritis pain, dental pain or diabetic peripheral neuropathy;
inflammatory pain is selected from pain associated with rheumatoid arthritis, osteoarthritis, rheumatoid spondylitis, gouty arthritis or juvenile arthritis;
musculoskeletal pain selected from bone and joint pain, osteoarthritis; lower back and neck pain; pain resulting from physical trauma or amputation;
neurologic or neuronal injury associated or related pain disorders caused by diseases selected from neuropathy, pain associated nerve injury, pain associated root avulsions, painful traumatic mononeuropathy, painful polyneuropathy, erythromelalgia, paroxysmal extreme pain disorder (PEPD), burning mouth syndrome; central pain syndromes caused by a lesion at a level of nervous system); traumatic nerve injury, nerve compression or entrapment, congenital insensitivity to pain (CIP), dysmenorrheal, primary erythromelalgia; HIV peripheral sensory neuropathy; pudendal neuralgia, spinal nerve injury, chronic inflammatory demyelinating polyneuropathy (CIDP), carpal tunnel syndrome or vasculitic neuropathy; or
inflammatory pain of varied origins selected from:
osteoarthritis, rheumatoid arthritis, rheumatic disorder, teno-synovitis and gout, shoulder tendonitis or bursitis, gouty arthritis, and polymyalgia rheumatica, primary hyperalgesia, secondary hyperalgesia, primary allodynia, secondary allodynia, or other pain caused by central sensitization; complex regional pain syndrome, chronic arthritic pain and related neuralgias or acute pain.

17. The method according to claim 16, wherein:
the pain-associated disease(s);
the pain caused by trauma; or
the pain caused by iatrogenic, medical or dental procedures, respectively,
is selected from:
chronic, acute or pre-operative associated pain; or
acute, chronic or post-operative associated pain.

18. The method according to claim 16, wherein:
the chronic, acute or pre-operative associated pain is selected from neuropathic pain or chronic neuropathic pain, chronic osteoarthritis pain, dental pain or inflammatory pain; or
the acute, chronic or post-operative associated pain is selected from bunionectomy pain, hernia repair pair, breast surgery pain or cosmetic surgical pain.

19. The method according to claim 18, wherein:
neuropathic pain or chronic neuropathic pain is selected from small fiber-mediated diabetic neuropathy, small fiber neuropathy, idiopathic small fiber neuropathy, painful diabetic neuropathy or polyneuropathy; or
inflammatory pain selected from osteoarthritis, chronic osteoarthritis knee pain or chronic inflammatory demyelinating polyneuropathy.

20.-30. (canceled)

31. The compound according to claim 1 which is selected from:
1-(4-fluoro-2-methylphenyl)-3-(2-methyl-6-oxo-1,6-dihydropyridin-3-yl)-6-(trifluoromethyl)-2,3-dihydropyrido[2,3-d]pyrimidin-4(1H)-one;
1-(4-fluoro-2-methylphenyl)-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-3-(6-oxo-1,6-dihydropyridin-3-yl)-6-(trifluoromethyl)-2,3-dihydroquinazolin-4(1H)-one;
1-(2-methyl-4-(trifluoromethoxy)phenyl)-3-(2-methyl-6-oxo-1,6-dihydropyridin-3-yl)-6-(trifluoromethyl)-2,3-dihydropyrido[2,3-d]pyrimidin-4(1H)-one;
1-(4-bromo-2-methylphenyl)-3-(6-oxo-1,6-dihydropyridin-3-yl)-7-(trifluoromethyl)-2,3-dihydroquinazolin-4(1H)-one;
1-(4-fluorophenyl)-3-(2-methyl-6-oxo-1,6-dihydropyridin-3-yl)-7-(trifluoromethyl)-2,3-dihydroquinazolin-4(1H)-one;
1-(4-fluoro-2-methylphenyl)-3-(2-methyl-6-oxo-1,6-dihydropyridin-3-yl)-6-(trifluoromethyl)-2,3-dihydroquinazolin-4(1H)-one; and
3-methyl-4-(3-(2-methyl-6-oxo-1,6-dihydropyridin-3-yl)-4-oxo-7-(trifluoromethyl)-3,4-dihydroquinazolin-1(2H)-yl)benzonitrile;
or a tautomer thereof,
or a pharmaceutically acceptable salt thereof.

32. A compound which is: 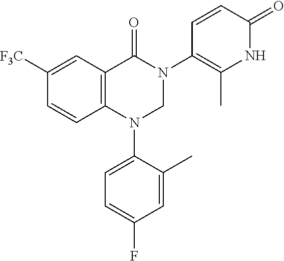 [SMILES: Cc1cc(F)ccc1N1CN(c2ccc(=O)[nH]c2C)C(=O)c2cc(C(F)(F)F)ccc21]
or a tautomer thereof,
or a pharmaceutically acceptable salt thereof.
